(19) United States
(12) Patent Application Publication
(10) Pub. No.: US 2025/0144223 A1
(43) Pub. Date: May 8, 2025

(54) PROTEIN DEGRADATION COMPOUND BASED ON NAMPT TARGET PROTEIN AND APPLICATION THEREOF

(71) Applicants: GLUETACS THERAPEUTICS (SHANGHAI) CO., LTD., Shanghai (CN); ShanghaiTech University, Shanghai (CN)

(72) Inventors: Xiaobao Yang, Shanghai (CN); Gaofeng Fan, Shanghai (CN); Biao Jiang, Shanghai (CN); Haixia Liu, Shanghai (CN); Xiaotong Zhu, Shanghai (CN); Renhong Sun, Shanghai (CN)

(73) Assignees: GLUETACS THERAPEUTICS (SHANGHAI) CO., LTD., Shanghai (CN); ShanghaiTech University, Shanghai (CN)

(21) Appl. No.: 18/689,031

(22) PCT Filed: Aug. 25, 2022
(86) PCT No.: PCT/CN2022/114935

§ 371 Date: Mar. 4, 2024

(30) Foreign Application Priority Data
Sep. 2, 2021 (CN) 202111025519.1

(51) Int. Cl. A61K 47/55; A61K 47/54; A61P 35/00

(52) U.S. Cl. CPC A61K 47/55; A61K 47/545; A61P 35/00

(57) ABSTRACT

The present disclosure provides compounds of Formula (I) or a salt, enantiomer, stereoisomer, solvate, isotopically enriched analog, prodrug, or polymorph thereof, and uses thereof. The present disclosure also provides pharmaceutical compositions comprising, as an active ingredient, the compound of Formula (I) or a salt, enantiomer, stereoisomer, solvate, isotopically enriched analog, prodrug, or polymorph thereof, and uses thereof. The series of compounds designed and synthesized in the present disclosure can effectively prevent and/or treat diseases or disorders associated with NAMPT.

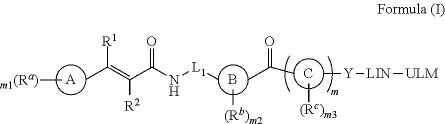

### TECHNICAL FIELD

The present disclosure relates to protein degradation compounds of Formula (I) designed based on nicotinamide phosphoribosyltransferase (NMPRTase; NAMPT) target protein or salts, enantiomers, stereoisomers, solvates, prodrugs or polymorphs thereof, as well as theirs use in the treatment or prevention of diseases or disorders associated with NAMPT.

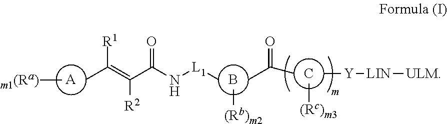

### BACKGROUND

Nicotinamide adenine dinucleotide (NAD⁺) as an enzyme cofactor for transferring hydrogen participates in various physiological functions such as energy synthesis, cellular metabolism, DNA repair, etc^{[1]}. As an important small-molecule metabolite, nicotinamide adenine dinucleotide (NAD⁺) is widely involved in a series of biochemical reactions in cell energy metabolism, such as glycolysis, oxidative phosphorylation, and fatty acid oxidation. Therefore, NAD⁺ is crucial for human health^{[2]}.

There are three biosynthetic pathways for NAD^{+[3]}:

1. De novo pathway. This pathway starts with indoleamine 2,3-dioxygenase (IDO) or tryptophan 2,3-dioxygenase (TDO), which catalyze the conversion of tryptophan into N-formylkynurenine. Then, N-formylkynurenine is converted by formamidase (KFase) to kynurenine which is then hydroxylated by kynurenine 3-hydroxylase (K3H) to produce 3-hydroxykynurenine. The product 3-hydroxykynurenine is further converted into 3-hydroxyanthranilic acid which is then converted into 2-amino-3-carboxymuconate-6-semialdehyde (ACMS) by kynureninase (Kyase) and 3-hydroxyanthranilate 3,4-dioxygenase^{[4]}. ACMS then cyclizes to form quinolinic acid (QA) that participates in NAMN biosynthesis with quinolinate phosphoribosyltransferase (QPRT)^{[5,6]}.

2. Preiss-Handler pathway. This pathway starts with the conversion of nicotinic acid (NA) to nicotinamide mononucleotide (NAMN) by Nicotinate Phosphoribosyltransferase (NAPRT)^{[7]}. NAMN is then used for the biosynthesis of nicotinic acid adenine dinucleotide (NAAD) by nicotinamide/nicotinic acid mononucleotide adenylyltransferase (NMNAT). Finally, NAD⁺ synthase (NADS) transforms the NAAD into NAD⁺ with ammonia and ATP action^{[8-9]}.

3. Salvage pathway. Rather than generated de novo pathway, most NAD⁺ is recycled from NAM, NA, NR and NMN in the salvage pathway to maintain the cellular NAD⁺ levels. Rather than generated de novo pathway, most NAD⁺ is recycled from NAM, NA, NR and NMN in the salvage pathway to maintain the cellular NAD⁺ levels. Therefore, the salvage pathway is the primary source of NAD⁺ in mammalian cells. NAM could be recycled from NAD+ consumption reactions, including both NAD⁺-dependent deacylation and ADP-ribosylation, into NMN by NAMPT, which catalyzes the rate-limiting reaction in the salvage pathway^{[10]}. The precursor NR is transformed to NMN by NRK1/2. Ultimately, NMN is adenylated by NMNAT to yield NAD^{+[2]}.

Nicotinamide phosphoribosyltransferase (NAMPT) is the rate-limiting enzyme that catalyzes the synthesis of nicotinamide adenine dinucleotide (NAD⁺).

Cancer cells have different metabolic demands and increased NAD⁺ turnover as compared to normal cells^{[11]}. Therefore, the salvage pathway is of major importance to cancer cells. In fact, many types of cancer cells have been shown to highly express NAMPT, reflecting potentially increased dependence on this pathway due to high NAD⁺ utilization and in some cases, loss of expression of other key NAD⁺ biosynthetic enzymes^{[12-15]}. The types of cancers reported to have high NAMPT expression include, but are not limited to, colorectal cancer (CRC), breast cancer, osteosarcoma, chondrosarcoma, pancreatic ductal adenocarcinoma, oral squamous cell carcinoma, prostate cancer, rhabdomyosarcoma, leiomyosarcoma, esophagogastric junction adenocarcinomas, thyroid cancer, leukemia, lymphoma, ovarian cancer, and some renal cancer, and in many of these, higher NAMPT expression correlated with worse outcomes^{[16-32]}. NMN may also be produced from nicotinamide riboside via nicotinamide riboside kinase^{[13]}. Currently, however, NAMPT is the only NAD⁺ production enzyme that has been targeted in the clinic.

NAMPT exists in two forms in mammals: intracellular NAMPT (iNAMPT) in the cytoplasm and nucleus and extracellular NAMPT (eNAMPT) in the plasma or extracellular space.

NAMPT is overexpressed in several human malignancies such as colorectal cancer, ovarian cancer, breast cancer, gastric cancer, prostate cancer, endometrial cancer, melanoma, multiple myeloma, astrocytoma, liver cancer, thyroid cancer, malignant lymphoma, and others. Given its role in promoting various aspects of the malignant phenotype, inhibiting NAMPT may have anticancer effects. NAMPT inhibition also leads to ATP depletion, reduced PARP-1 and SirT1 activity, and ultimately cell death^{[34-36]}. Tumor cells are more sensitive to iNAMPT inhibition than normal cells due to increased NAD⁺ and ATP catabolism^{[37]}. The highly specific NAMPT inhibitor FK866 can induce apoptosis in human hepatocellular carcinoma cells (HepG2) by depleting NAD⁺, which could be partially reversed by adding NAM or nicotinic acid^{[33]}. NAMPT inhibitors have potential therapeutic applications in treating triple-negative breast cancer, liver cancer, gastric cancer, non-small cell lung cancer with epidermal growth factor receptor gene mutations, glioblastoma, melanoma, etc.

eNAMPT can be released by various types of cells and acts as a cytokine on multiple cell types. It is able to activate downstream intracellular pathways by stimulating the subsequent release of other cytokines.

Plasma eNAMPT levels are elevated in various human malignancies, including astrocytoma, myeloma, male oral squamous cell carcinoma, gastric cancer, endometrial cancer, hepatocellular carcinoma, colorectal cancer, and invasive breast cancer, etc^{[38-46]}. In astrocytoma, plasma eNAMPT levels increase with tumor staging and are considered a prognostic marker. Similarly, eNAMPT levels are elevated in tumor later stages of male oral squamous cell carcinoma, hepatocellular carcinoma, endometrial cancer, and infiltrating breast cancer. In patients with endometrial cancer, higher eNAMPT levels are associated with endometrial infiltrating and shorter patient survival. Higher eNAMPT levels in infiltrating breast cancer are associated with lymph node metastasis and the loss of estrogen and progesterone receptors. Myocardial cells treated with H₂O₂ or incubated in the condition of serum starvation will secrete eNAMPT^{[47]}. Pretreatment of human chondrocytes with eNAMPT may inhibit IGF-1-stimulated proteoglycan synthesis and AKT and insulin receptor substrate-1 phosphorylation while activating ERK^{[48]}. Researchers have found that eNAMPT treatment rapidly induces the production of IL-6 in mouse macrophages, followed by IL-6-mediated STAT3 activation. Interestingly, IL-1β, TNF-α, and IL-6 can induce the expression of eNAMPT in macrophages^{[49-51]}. These data suggest that plasma eNAMPT may contribute to carcinogenesis and tumor growth.

NAMPT is also closely related to the occurrence and development of various diseases.

Diabetic nephropathy is one of the most severe diabetic microvascular complications and a risk factor for death in diabetes patients, posing a serious threat to human health. The pathogenesis of diabetic nephropathy is complex and diverse, mainly involving renal inflammation and fibrosis. Studies have found that the renal inflammatory and fibrotic process is closely related to NAMPT. On the one hand, in glomerular mesangial cells, NAMPT can promote the expression and translocation of GLUT-1 to the cell membrane, thereby mediating intracellular glucose transport, leading to increased glucose metabolism and extracellular matrix synthesis, which causes renal parenchymal damage^{[52,53]}. On the other hand, diabetic patients are in an obvious imbalance state of oxidative stress. By detecting the patient's serum, it is found that serum levels of NAMPT are directly proportional to lipid peroxidation metabolite levels and inversely proportional to antioxidant enzyme SOD levels, indicating a close relationship between NAMPT and oxidative stress in vivo^{[54]}. The interaction of these two factors together promotes the occurrence of diabetic nephropathy.

NAMPT is closely related to the occurrence and development of cardiovascular and cerebrovascular diseases. NAMPT expression is significantly increased in carotid plaques of patients with unstable coronary atherosclerosis and ruptured plaques in patients with acute myocardial infarction^{[55,56]}. Studies have found that NAMPT can enhance the enzymatic activity of MMP-2 and MMP-9, while MMP can degrade the extracellular matrix, which leads to a thin fibrous cap and plaque rupture, suggesting that NAMPT plays an important role in the instability of atherosclerotic plaques^{[57]}. NAMPT inhibitors can reduce myocardial infarction size, neutrophil infiltration, and the production of reactive oxygen species generation within a mouse model of myocardial ischemia-reperfusion injury^{[58]}. Therefore, as an effective therapeutic drug, NAMPT pharmacological inhibitors can reduce oxidative stress-mediated tissue damage in myocardial infarction.

NAMPT is overexpressed in placental tissue from women with gestational hypertension, and the expression levels of both are significantly positively correlated, suggesting a close relationship with gestational hypertension.

Clinical studies have shown that the concentration of NAMPT is significantly increased in chronic inflammatory diseases. As a pro-inflammatory adipokine, NAMPT promotes the expression of monocyte chemoattractant protein-1 and intercellular adhesion molecule-1 protein^{[59]}. NAMPT can induce CD14⁺ monocytes to produce inflammatory cytokines such as interleukin-1β, tumor necrosis factor-α, and interleukin-6, and increase the surface expression of co-stimulatory molecules CD54, CD40, and CD80^{[60]}. eNAMPT can activate NF-κB, leading to upregulation of inducible nitric oxide synthase expression and promotion of inflammatory responses^{[61]}.

NAMPT also plays a role as an immune regulatory cytokine. The development and function of T lymphocytes and B lymphocytes are almost completely dependent on NAMPT. Conditional knockout of the NAMPT gene in lymphocytes leads to a significant decrease in thymocyte numbers and almost complete loss of peripheral blood T lymphocytes and B lymphocytes^{[62]}. High levels of NAMPT can be detected in the serum of patients with autoimmune diseases such as rheumatoid arthritis and autoimmune encephalitis^{[63,64]}.

The high expression of NAMPT in blood and gastric adipose tissue is positively correlated with overweight or obesity. NAMPT is a necessary factor for overweight or obesity, and downregulating NAMPT in adipose tissue can help alleviate obesity^{[65]}.

Currently, the NAMPT inhibitor FK866 is in the clinical stage. It can exert antitumor effects in cell models and animal models, especially in hematological tumors. FK866 can clear tumor cells to undetectable levels, resulting in long-term survival of 80% of mice^{[66]}. However, there are some issues with FK866. Clinical trial results have shown that FK866 treatment can cause dose-limiting toxicities such as thrombocytopenia, as well as gastrointestinal reactions such as diarrhea and constipation^{[67]}. This is also the reason why the phase II clinical trial utilizing FK866 was halted. In addition, NAMPT exists in both enzymatically active and inactive forms. FK866, as an enzyme inhibitor, cannot inhibit the non-enzymatically active form of NAMPT (eNAMPT) in plasma and extracellular spaces. eNAMPT has been shown to contribute to carcinogenesis and tumor growth, which may affect the therapeutic effect of FK866. Therefore, there is a need to improve existing inhibitors or develop new drugs to overcome the deficiencies of existing inhibitors.

Designing degraders targeting specific proteins is a new model for drug development. We utilize the Proteolysis Targeting Drug (PROTAD) technology platform for the development of antitumor drugs. PROTAD can label target proteins for degradation through specially designed bispecific protein regulators, and degrade target proteins by activating intracellular protein degradation pathways. Therefore, we hope to develop protein modulators that can degrade disease-related pathogenic proteins using the protein degradation technology platform, thereby treating cancer patients and prolonging their survival.

Therefore, there is an urgent need for a series of novel protein degraders designed based on NAMPT target protein for the treatment and/or prevention of diseases or disorders associated with NAMPT.

### SUMMARY OF INVENTION

The present disclosure provides series of novel NAMPT target protein based-protein degraders, salts, solvates, isotopically enriched analogs, polymorphs, prodrugs, stereoisomers (including enantiomers), or mixture of stereoisomers thereof, which can target and degrade NAMPT protein, effectively treating and/or preventing diseases or disorders associated with NAMPT. The diseases or disorders associated with NAMPT include but are not limited to tumors, autoimmune diseases, inflammatory diseases, gestational hypertension, cardiovascular and cerebrovascular diseases, obesity and diabetic nephropathy.

In some embodiments, the present disclosure provides a compound of Formula (I) or a salt, solvate, isotopically enriched analog, polymorph, prodrug, stereoisomer (including enantiomer), or mixture of stereoisomers thereof:

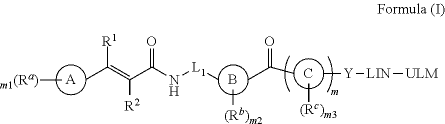

wherein ULM is a E3 ligase ligand moiety, LIN is a linker moiety, the remaining moiety of the compound of Formula (I) is a nicotinamide phosphoribosyltransferase (NAMPT) ligand, and ULM is covalently bonded to NAMPT ligand through LIN;

wherein ring A is the following groups:

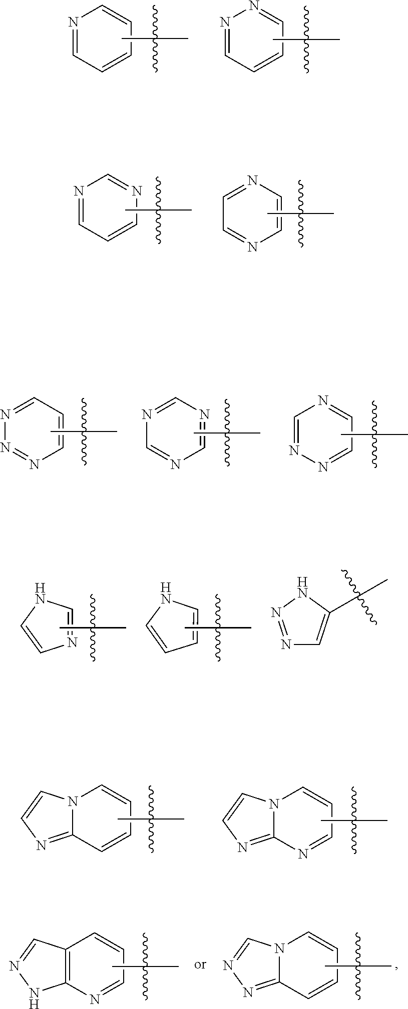

(R^{a})ₘ₁ indicates that the ring A is optionally substituted by m1 R^{a} groups, wherein each R^{a} is independently hydroxyl, amino, halogen, or cyano, and m1 represents an integer of 0, 1, 2, 3, 4, or 5;
R¹ and R² are the same or different and each independently represent H, cyano, or methyl;
L₁ represents substituted or unsubstituted linear C₃₋₆ alkylene group, or substituted or unsubstituted linear C₃₋₆ alkenylene group, wherein the optional substituents of the linear C₃₋₆ alkylene group and the linear C₃₋₆ alkenylene group are selected from the group consisting of C₁₋₃ alkyl, halogen, C₁₋₃ alkoxy, halogenated C₁₋₃ alkyl, C₁₋₃ alkyl-NHC(O)—, C₁₋₃ alkyl-C(O)NH—, cyano, or any combination thereof;
ring B is a 5- to 11-membered heterocyclylene group, and (R^{b})ₘ₂ indicates that ring B is optionally substituted by m2 R^{b} groups, wherein each R^{b} is independently C₁₋₃ alkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, or cyano, and m2 represents an integer of 0, 1, 2, 3, 4, or 5;
ring C is a 5- to 10-membered heteroarylene group or a 6-membered arylene group, and (R^{c})ₘ₃ indicates that ring C is optionally substituted by m3 R^{c} groups, wherein each R^{c} is independently C₁₋₃ alkyl, C₃₋₅ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, or cyano, and m3 represents an integer of 0, 1, 2, 3, 4, or 5;
m represents an integer of 0 or 1; and
Y represents —N(R³)—, where R³ is H or C₁₋₃ alkyl; or
Y represents —O—; or
Y is n connected rings D represented by the following formula:

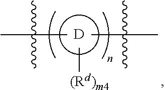

each ring D is independently a 5- to 11-membered heterocyclylene group, and (R^{d})ₘ₄ indicates that each ring D is optionally independently substituted by m4 R^{d} groups, wherein each R^{d} is independently C₁₋₃ alkyl, C₃₋₆ cycloalkyl, hydroxyl, amino, mercapto, halogen, oxo, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, or cyano, and m4 represents an integer of 0, 1, 2, 3, 4, or 5;
n represents an integer of 0, 1, 2, or 3, wherein when n represents an integer of 2 or 3, each ring D can be the same or different, and
where m and n are not both 0 at the same time;
with the proviso that the following compounds are excluded:
the nicotinamide phosphoribosyltransferase (NAMPT) ligand represents the following structure:

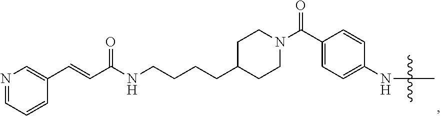 [SMILES: *Nc1ccc(C(=O)N2CCC(CCCCNC(=O)/C=C/c3cccnc3)CC2)cc1]

and
ULM represents a structure of Formula (IV):

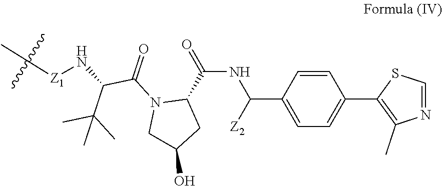

wherein Z₁ represents C(O) or Z₁ represents a bond, and Z₂ represents H or CH₃.

In some embodiments, the present disclosure provides a pharmaceutical composition comprising as active ingredient the compound of Formula (I) or a pharmaceutically acceptable salt, solvate, isotopically enriched analog, polymorph, prodrug, stereoisomer (including enantiomer), or mixture of stereoisomers thereof, and at least one pharmaceutically acceptable carrier.

In some embodiments, the present disclosure provides a medicine kit or reagent kit comprising the compound of Formula (I) or a pharmaceutically acceptable salt, solvate, isotopically enriched analog, polymorph, prodrug, stereoisomer (including enantiomer), or mixture of stereoisomers thereof of the present disclosure, or the pharmaceutical composition of the present disclosure.

In some embodiments, the present disclosure provides the compound of Formula (I) or a pharmaceutically acceptable salt, solvate, isotopically enriched analog, polymorph, prodrug, stereoisomer (including enantiomer), or mixture of stereoisomers thereof for use as a medicament.

In some embodiments, the present disclosure provides the compound of Formula (I) or a pharmaceutically acceptable salt, solvate, isotopically enriched analog, polymorph, prodrug, stereoisomer (including enantiomer), or mixture of stereoisomers thereof, or the pharmaceutical composition of the present disclosure for use in the prevention and/or treatment of a disease or disorder associated with NAMPT.

In some embodiments, the present disclosure provides use of the compound of Formula (I) or a pharmaceutically acceptable salt, solvate, isotopically enriched analog, polymorph, prodrug, stereoisomer (including enantiomer), or mixture of stereoisomers thereof, or the pharmaceutical composition of the present disclosure for the manufacture of a medicament for the prevention and/or treatment of a disease or disorder associated with NAMPT.

In some embodiments, the present disclosure provides a method for treating or preventing a disease or disorder associated with NAMPT in a subject, comprising administering to the subject a therapeutically effective amount of the compound of Formula (I) or a pharmaceutically acceptable salt, solvate, isotopically enriched analog, polymorph, prodrug, stereoisomer (including enantiomer), or mixture of stereoisomers thereof, or the pharmaceutical composition of the present disclosure.

### BRIEF DESCRIPTION OF DRAWINGS

FIG. 1 shows the degradation effects of compounds SIAIS630120 and SIAIS630121 of the present disclosure on NAMPT protein in SW620 and HT29 cell lines.

FIG. 2 shows the degradation effects of compounds SIAIS630120 and SIAIS630121 of the present disclosure on NAMPT protein in MCF-7 cell line.

FIG. 3 shows the degradation effects of compounds SIAIS630120 and SIAIS630121 of the present disclosure on NAMPT protein in BEL7404 cell line.

FIG. 4 shows the degradation effects of compounds SIAIS630120 and SIAIS630121 of the present disclosure on NAMPT protein in MOLT4, Jurkat, and HL60 cell lines.

FIG. 5 shows the degradation effects of compounds SIAIS630120 and SIAIS630121 of the present disclosure on eNAMPT protein in HL60 and MOLT4 cell lines.

FIGS. 6-7 show the in vitro inhibition effects of compounds SIAIS630120 and SIAIS630121 of the present disclosure, as well as the parent inhibitor FK866, on HL60 and MOLT4 cell lines.

### DETAILED DESCRIPTION OF THE INVENTION

The following detailed description is provided as exemplary specific embodiments to assist those skilled in the art in understanding and practicing the present disclosure. It should be appreciated, however, that such description is not intended to limit the scope of the present disclosure, and that various modifications and changes may be made to the specific embodiments described in the present disclosure without departing from the spirit and scope of the present disclosure. Such changes and modifications are to be understood as being included within the scope of the present invention as defined by the appended claims.

### I. Compounds

The present disclosure provides a compound of Formula (I) or a salt (including a pharmaceutically acceptable salt), solvate, isotopically enriched analog, polymorph, prodrug, stereoisomer (including enantiomer), or mixture of stereoisomers thereof:

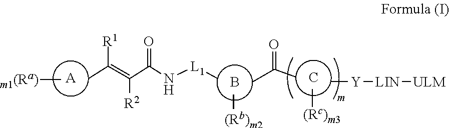

wherein ULM is a E3 ligase ligand moiety, LIN is a linker moiety, the remaining moiety of the compound of Formula (I) is a nicotinamide phosphoribosyltransferase (NAMPT) ligand, and ULM is covalently bonded to NAMPT ligand through LIN;
wherein ring A is the following groups:

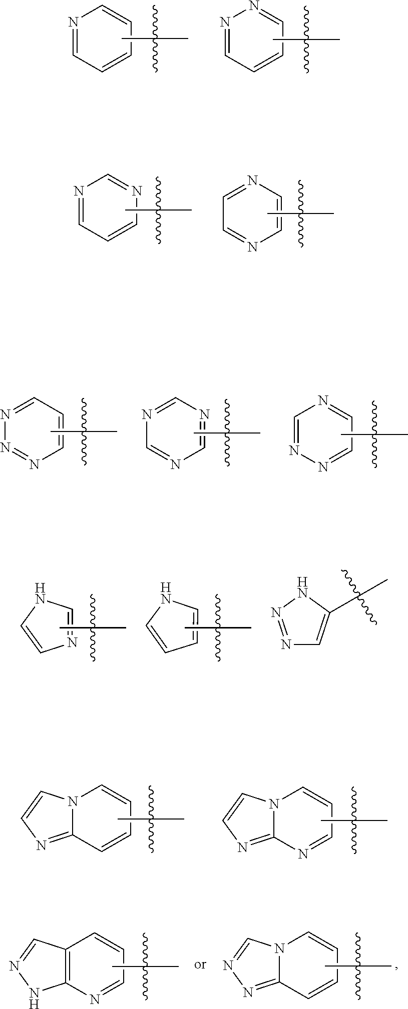

(R^{a})ₘ₁ indicates that the ring A is optionally substituted by m1 R^{a} groups, wherein each R^{a} is independently hydroxyl, amino, halogen, or cyano, and m1 represents an integer of 0, 1, 2, 3, 4, or 5;
R¹ and R² are the same or different and each independently represent H, cyano, or methyl;
L₁ represents substituted or unsubstituted linear C₃₋₆ alkylene group, or substituted or unsubstituted linear C₃₋₆ alkenylene group, wherein the optional substituents of the linear C₃₋₆ alkylene group and the linear C₃₋₆ alkenylene group are selected from the group consisting of C₁₋₃ alkyl, halogen, C₁₋₃ alkoxy, halogenated C₁₋₃ alkyl, C₁₋₃ alkyl-NHC(O)—, C₁₋₃ alkyl-C(O)NH—, cyano, or any combination thereof;
ring B is a 5- to 11-membered heterocyclylene group, and (R^{b})ₘ₂ indicates that ring B is optionally substituted by m2 R^{b} groups, wherein each R^{b} is independently C₁₋₃ alkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, or cyano, and m2 represents an integer of 0, 1, 2, 3, 4, or 5;
ring C is a 5- to 10-membered heteroarylene group or a 6-membered arylene group, and (R^{c})ₘ₃ indicates that ring C is optionally substituted by m3 R^{c} groups, wherein each R^{c} is independently C₁₋₃ alkyl, C₃₋₅ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, or cyano, and m3 represents an integer of 0, 1, 2, 3, 4, or 5;
m represents an integer of 0 or 1; and
Y represents —N(R³)—, where R³ is H or C₁₋₃ alkyl; or
Y represents —O—; or
Y is n connected rings D represented by the following formula:

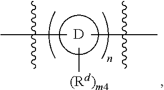

each ring D is independently a 5- to 11-membered heterocyclylene group, and (R^{d})ₘ₄ indicates that each ring D is optionally independently substituted by m4 R^{d} groups, wherein each R^{d} is independently C₁₋₃ alkyl, C₃₋₆ cycloalkyl, hydroxyl, amino, mercapto, halogen, oxo, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, or cyano, and m4 represents an integer of 0, 1, 2, 3, 4, or 5;
n represents an integer of 0, 1, 2, or 3, wherein when n represents an integer of 2 or 3, each ring D can be the same or different, and
where m and n are not both 0 at the same time;
with the proviso that the following compounds are excluded:
the nicotinamide phosphoribosyltransferase (NAMPT) ligand represents the following structure:

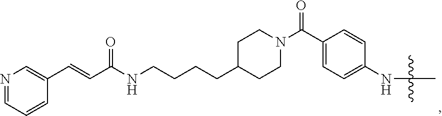 [SMILES: *Nc1ccc(C(=O)N2CCC(CCCCNC(=O)/C=C/c3cccnc3)CC2)cc1]

and
ULM represents a structure of Formula (IV):

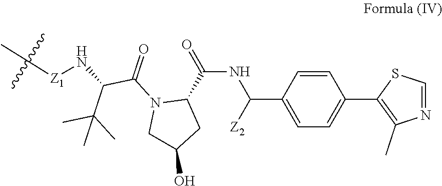

wherein Z₁ represents C(O) or Z₁ represents a bond, and Z₂ represents H or CH₃.

In some embodiments, L₁ represents substituted or unsubstituted linear C₃₋₆ alkylene group. Exemplary examples of linear C₃₋₆ alkylene group include, but are not limited to, propylene, butylene, pentylene, and hexylene. The linear C₃₋₆ alkylene group is optionally further substituted with one or more substituents selected from the group consisting of C₁₋₃ alkyl (e.g., methyl, ethyl, or propyl), halogen (e.g., fluorine, chlorine, bromine, or iodine), C₁₋₃ alkoxy (e.g., methoxy, ethoxy, or propoxy), halo-substituted C₁₋₃ alkyl (e.g., —CF₃, —CH₂F, —CHF₂, —CH₂Cl, —CHCl₂, —CF₂CF₃, —CHFCF₃, —CF₂CHF₂, —CHFCHF₂, —CH₂CF₃, and —CH₂CH₂Cl), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), cyano, or any combination thereof. The number of substituents is in principle not limited in any way, or is automatically limited by the size of the building unit.

In some embodiments, L₁ represents substituted or unsubstituted linear C₃₋₆ alkenylene group. Exemplary examples of linear C₃₋₆ alkenylene group include, but are not limited to,

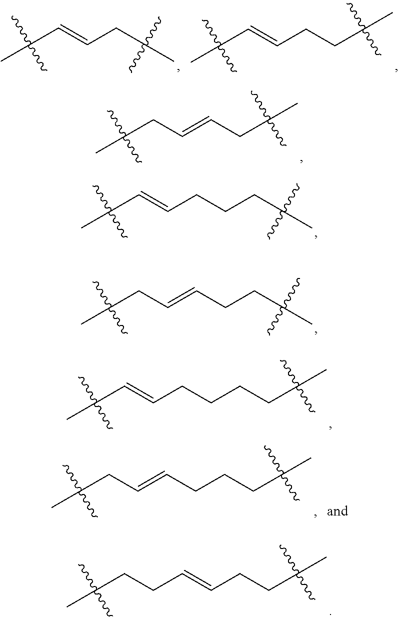

The linear C₃₋₆ alkenylene is optionally further substituted with one or more substituents selected from the group consisting of C₁₋₃ alkyl (e.g., methyl, ethyl, or propyl), halogen (e.g., fluorine, chlorine, bromine, or iodine), C₁₋₃ alkoxy (e.g., methoxy, ethoxy, or propoxy), halo-substituted C₁₋₃ alkyl (e.g., —CF₃, —CH₂F, —CHF₂, —CH₂Cl, —CHCl₂, —CF₂CF₃, —CHFCF₃, —CF₂CHF₂, —CHFCHF₂, —CH₂CF₃, and —CH₂CH₂Cl), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), cyano, or any combination thereof. The number of substituents is in principle not limited in any way, or is automatically limited by the size of the building unit.

In some embodiments, ring B is a 5- to 11-membered heterocyclylene containing from 1 to 3 heteroatoms independently selected from sulfur, oxygen, and nitrogen.

In some embodiments, ring B is a 5- to 11-membered heterocyclylene group containing 1 nitrogen atom and optionally containing from 1 to 2 heteroatoms independently selected from the group consisting of nitrogen, oxygen, and sulfur.

In some embodiments, exemplary examples of ring B include, but are not limited to, the following groups: piperidinylene, piperazinylene, morpholinylene, azetidinylene, oxetanylene, pyrrolidinylene, imidazolidylene, pyrazolidylene, tetrahydrofuranylene, tetrahydropyranylene, tetrahydrothienylene, tetrahydrothiopyranylene, oxazolidinylene, thiazolidinylene, thiomorpholinylene, dioxanylene, diazepanylene, or C₇₋₁₁ spiroheterocyclylene.

In some embodiments, when ring B represents C₇₋₁₁ spiroheterocyclylene, the C₇₋₁₁ spiroheterocyclylene may be the following groups:

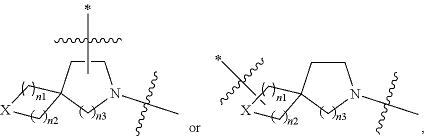

wherein the symbol * indicates the point of attachment to L₁ (which can indicate any ring atom on the spiroheterocyclic ring that can be connected to L₁), X represents O, N(R^{e}), S, or CH₂, or X represents a bond, where R^{e} represents H or methyl, and n1, n2, and n3 are each independently an integer of 1 or 2.

As used herein, the wording “ . . . represents a bond” used alone or in combination means that the referenced group is a bond linker (that is, the referenced group is absent). For example, the wording “X represents a bond” means that X is a bond linker. In other words, when X presents a bond, the two adjacent carbon atoms on both sides of X are directly connected to each other.

In some embodiments, when ring B represents C₇₋₁₁ spiroheterocyclylene, the C₇₋₁₁ spiroheterocyclylene may be the following groups:

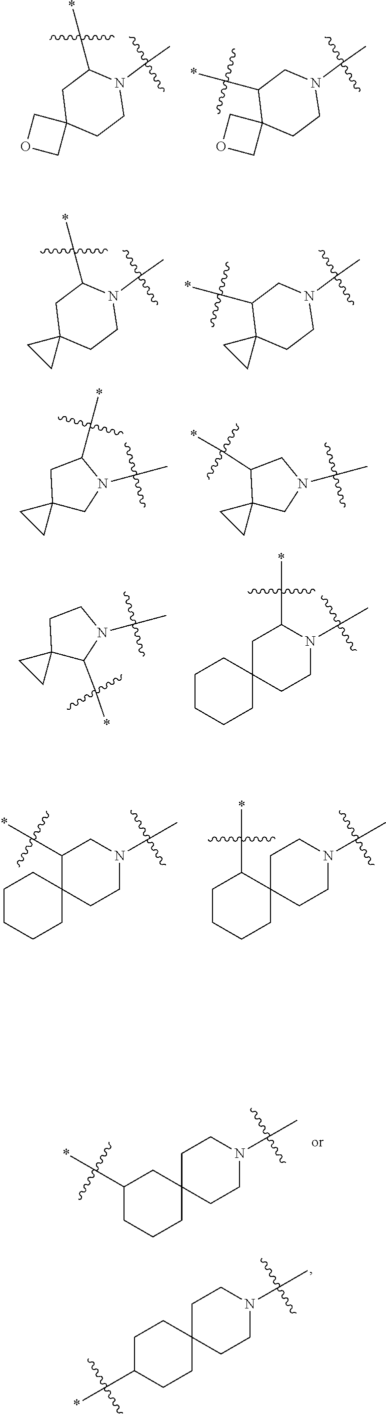

wherein the symbol * indicates the point of attachment to L₁.

In some embodiments, ring B is optionally further substituted by m2 R^{b} groups, wherein m2 represents an integer of 0, 1, 2, 3, 4, or 5, and each R^{b} is independently C₁₋₃ alkyl (e.g., methyl, ethyl, or propyl), hydroxyl, amino, mercapto, halogen (e.g., fluorine, chlorine, bromine, or iodine), C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), halogenated C₁₋₃ alkyl (e.g., —CF₃, —CH₂F, —CHF₂, —CH₂Cl, —CHCl₂, —CF₂CF₃, —CHFCF₃, —CF₂CHF₂, —CHFCHF₂, —CH₂CF₃, and —CH₂CH₂Cl), amino-substituted C₁₋₃ alkylene (NH₂—C₁₋₃ alkylene-, e.g., NH₂CH₂—, NH₂CH₂CH₂—, and NH₂CH₂CH₂CH₂—), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), or cyano.

In some embodiments, exemplary examples of

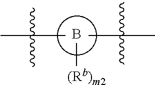

include, but are not limited to, the following groups:

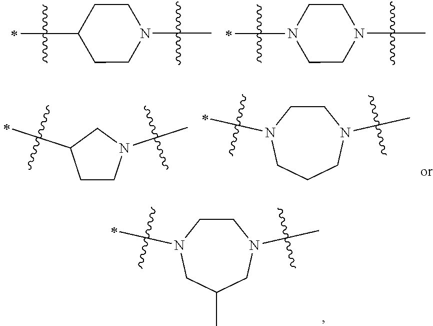

wherein the symbol * indicates the point of attachment to L₁.

In some embodiments, ring C is a 5- to 10-membered heteroarylene group or a 6-membered arylene group. Exemplary examples of ring C include, but are not limited to, the following groups: phenylene, pyridylene, pyrimidinylene, pyrazinylene, pyridazinylene, 1,2,4-triazinylene, 1,3,5-triazinylene, triazolylene, furanylene, oxazolylene, isoxazolylene, oxadiazolylene, thienylene, thiazolylene, isothiazolylene, thiadiazolylene, pyrrolylene, imidazolylene, pyrazolylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2a]pyridylene, 1H-pyrrolo[3,2-b]pyridylene, 1H-pyrrolo[2,3-b]pyridylene, 4H-fluoro[3,2b]pyrrolylene, pyrrolo[2,1-b]thiazolylene, or imidazo[2,1-b]thiazolylene.

In some embodiments, the ring C is optionally further substituted by m3 R^{c} groups, wherein m3 represents an integer of 0, 1, 2, 3, 4, or 5, and each R^{c} is independently C₁₋₃ alkyl (e.g., methyl, ethyl, or propyl), C₃₋₅ cycloalkyl (e.g., cyclopropyl, cyclobutyl, or cyclopentyl), hydroxyl, amino, mercapto, halogen (e.g., fluorine, chlorine, bromine, or iodine), C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), halogenated C₁₋₃ alkyl (e.g., —CF₃, —CH₂F, —CHF₂, —CH₂Cl, —CHCl₂, —CF₂CF₃, —CHFCF₃, —CF₂CHF₂, —CHFCHF₂, —CH₂CF₃, and —CH₂CH₂Cl), amino-substituted C₁₋₃ alkylene (NH₂—C₁₋₃ alkylene-, e.g., NH₂CH₂—, NH₂CH₂CH₂—, and NH₂CH₂CH₂CH₂—), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), or cyano.

In some embodiments, exemplary examples of

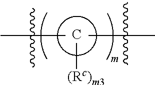

include, but are not limited to, the following groups:

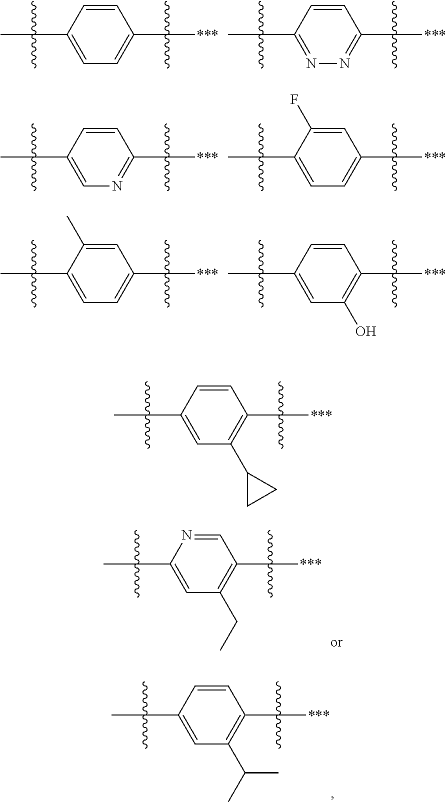

wherein the symbol *** indicates the point of attachment to Y, or the symbol *** indicates the point of attachment to carbonyl.

In some embodiments, Y represents —N(R³)—, where R³ is H or C₁₋₃ alkyl.

In some embodiments, Y represents —O—.

In some embodiments, Y is n connected rings D represented by the following formula:

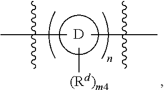

wherein n represents an integer of 0, 1, 2, or 3, wherein when n represents an integer of 2 or 3, each ring D can be the same or different, and each ring D is independently a 5- to 11-membered heterocyclylene group, and (R^{d})ₘ₄ indicates that each ring D is optionally independently substituted by m4 R^{d} groups, wherein m4 represents an integer of 0, 1, 2, 3, 4, or 5, and each R^{d} is independently C₁₋₃ alkyl, C₃₋₆ cycloalkyl, hydroxyl, amino, mercapto, halogen, oxo, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, or cyano.

In some embodiments, exemplary examples of ring D include, but are not limited to, the following groups: piperidinylene, piperazinylene, morpholinylene, azetidinylene, oxetanylene, pyrrolidinylene, imidazolidylene, pyrazolidylene, tetrahydrofuranylene, tetrahydropyranylene, tetrahydrothienylene, tetrahydrothiopyranylene, oxazolidinylene, thiazolidinylene, thiomorpholinylene, dioxanylene, diazepanylene, or C₇₋₁₁ spiroheterocyclylene.

In some embodiments, when ring D represents C₇₋₁₁ spiroheterocyclylene, the C₇₋₁₁ spiroheterocyclylene can be the following groups:

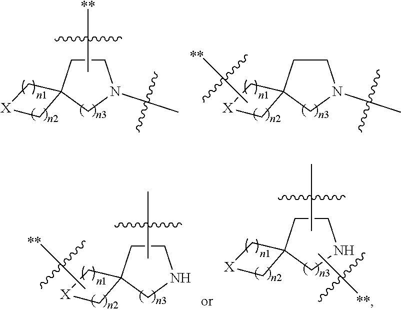

wherein the symbol ** indicates the point of attachment to ring C, X represents O, N(R^{e}), S, or CH₂, or X represents a bond, where R^{e} represents H or methyl, and n1, n2, and n3 are each independently an integer of 1 or 2.

In some embodiments, when ring D represents C₇₋₁₁ spiroheterocyclylene, the C₇₋₁₁ spiroheterocyclylene can be the following groups:

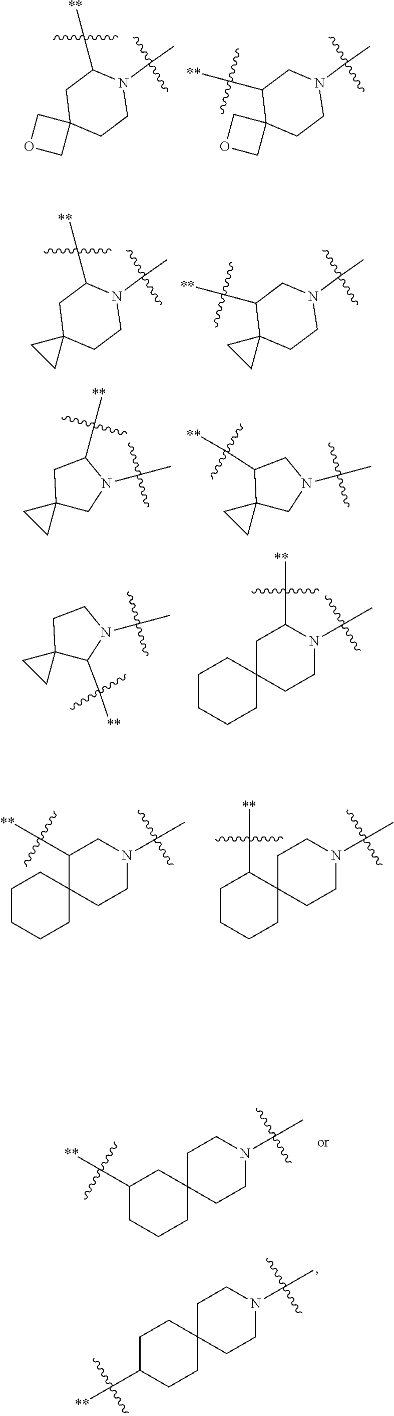

wherein the symbol ** indicates the point of attachment to ring C.

In some embodiments, ring D is optionally further substituted by m4 R^{d} groups, wherein m4 represents an integer of 0, 1, 2, 3, 4, or 5; and each R^{d} is independently C₁₋₃ alkyl (e.g., methyl, ethyl, or propyl), C₃₋₆ cycloalkyl (e.g., cyclopropyl, cyclobutyl, cyclopentyl, or cyclohexyl), hydroxyl, amino, mercapto, halogen (e.g., fluorine, chlorine, bromine, or iodine), oxo, C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), halogenated C₁₋₃ alkyl (e.g., —CF₃, —CH₂F, —CHF₂, —CH₂Cl, —CHCl₂, —CF₂CF₃, —CHFCF₃, —CF₂CHF₂, —CHFCHF₂, —CH₂CF₃, and —CH₂CH₂Cl), amino-substituted C₁₋₃ alkylene (NH₂—C₁₋₃ alkylene-, e.g., NH₂CH₂—, NH₂CH₂CH₂—, and NH₂CH₂CH₂CH₂—), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), or cyano.

In some embodiments, exemplary examples of

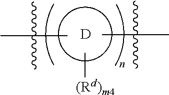

include, but are not limited to, the following groups:

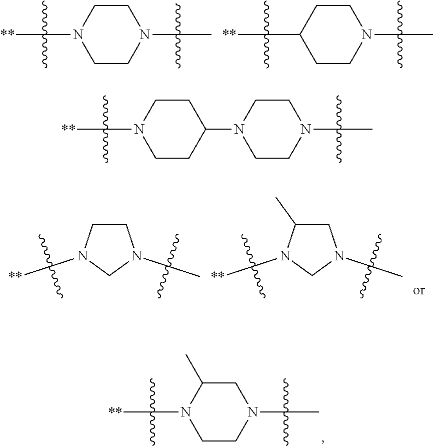

wherein the symbol ** indicates the point of attachment to ring C. Alternatively, in some embodiments, the symbol ** can also indicate the point of attachment to LIN.

In some embodiments, m is 0, n is 1. In some embodiments, m is 0, n is 2. In some embodiments, m is 0, n is 3. In some embodiments, m is 1, n is 0. In some embodiments, m is 1, n is 1. In some embodiments, m is 1, n is 2. In some embodiments, m is 1, n is 3.

In some embodiments, exemplary examples of

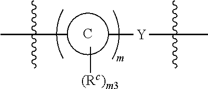

include, but are not limited to, the following groups:

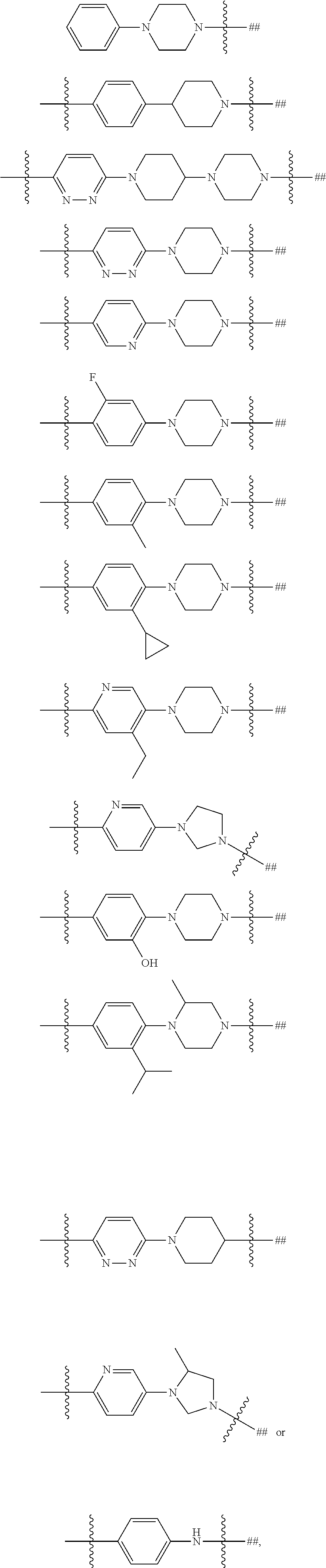

wherein the symbol ##indicates the point of attachment to LIN.

In some embodiments, ULM represents a structure of Formula (III):

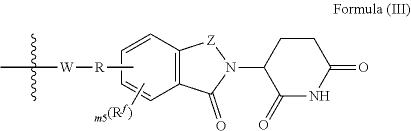

wherein R represents O, N(R⁴), S, alkynylene, alkenylene, or optionally substituted triazolylene, where R⁴ represents H or C₁₋₃ alkyl, or R represents a bond, and W represents optionally substituted phenylene or W represents a bond, wherein when R represents a bond, W represents a bond; and
(R^{f})ₘ₅ indicates that the phenyl ring in Formula (III) is optionally substituted by m5 R^{f} groups, wherein each R^{f} is independently halogen, m5 represents an integer of 0, 1, 2, or 3; and
Z represents C(O) or CH₂.

In some embodiments, the structure of Formula (III) also can be of the structure of the following formula:

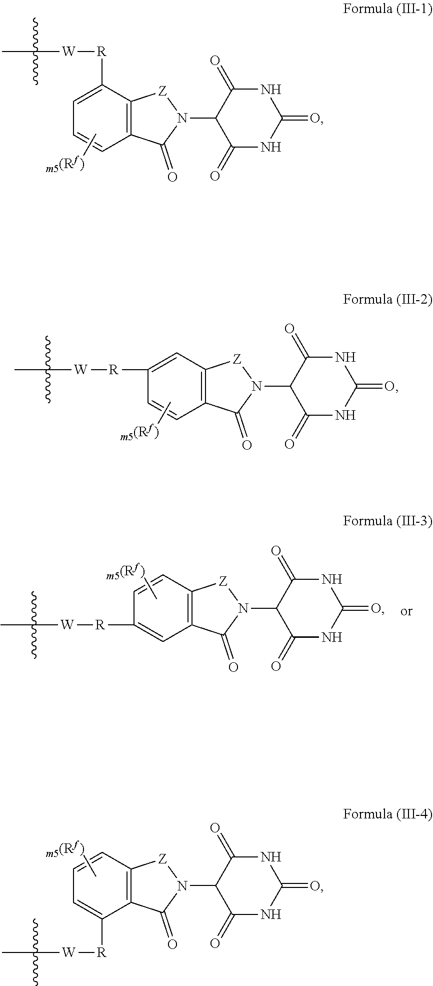

wherein Z represents C(O) or CH₂;
(R^{f})ₘ₅ indicates that the phenyl ring in Formula (III) is optionally substituted by m5 R^{f} groups, wherein each R^{f} is independently halogen, m5 represents an integer of 0, 1, 2, or 3; and
R represents O, N(R⁴), S, alkynylene, alkenylene, or optionally substituted triazolylene, where R⁴ represents H or C₁₋₃ alkyl, or R represents a bond, and W represents optionally substituted phenylene or W represents a bond, wherein when R represents a bond, W represents a bond.

In some embodiments, Z represents C(O).

In some embodiments, Z represents CH₂.

In some embodiments, R represents O.

In some embodiments, R represents N(R⁴), where R⁴ represents H or C₁₋₃ alkyl (e.g., methyl, ethyl, or propyl).

In some embodiments, R represents a bond. In some embodiments, when R represents a bond, W represents a bond.

In some embodiments, R represents alkynylene, e.g., ethynylene

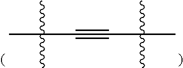 [SMILES: *C#C*]

or butynylene

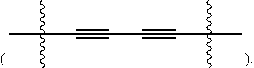 [SMILES: *C#CC#C*]

In some embodiments, R represents alkenylene, e.g., vinylene

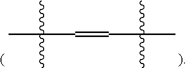 [SMILES: *C=C*]

In some embodiments, R represents S.

In some embodiments, R represents triazolylene.

In some embodiments, W represents optionally substituted phenylene, and exemplary examples of optional substituents include, but are not limited to, halogen (e.g., fluorine, chlorine, bromine, or iodine). The number of the optional substituents can be 0, 1, 2, or 3.

In some embodiments, W represents a bond.

In some embodiments, ULM represents the structure of the following formula:

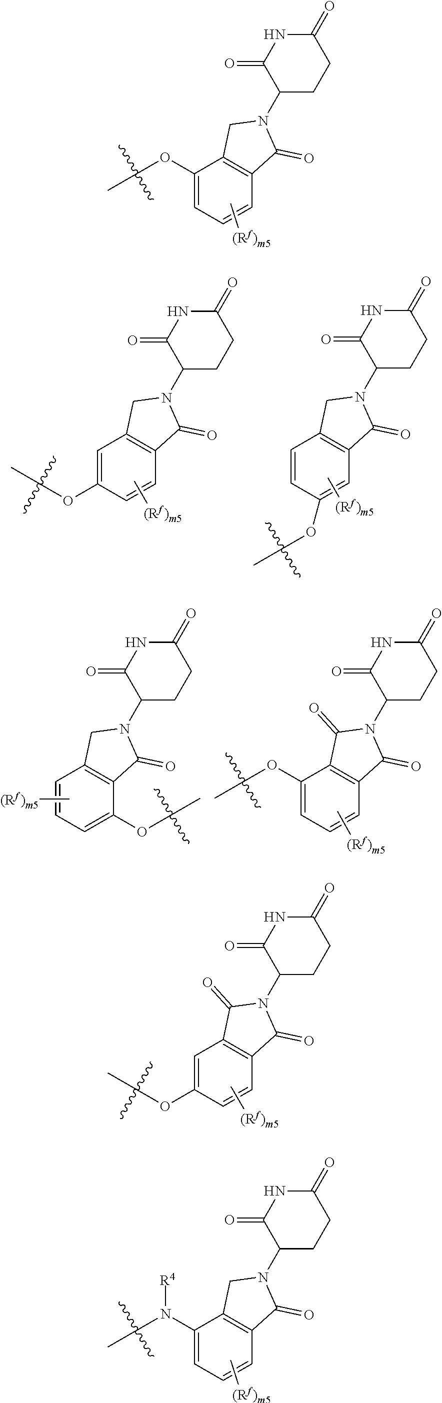

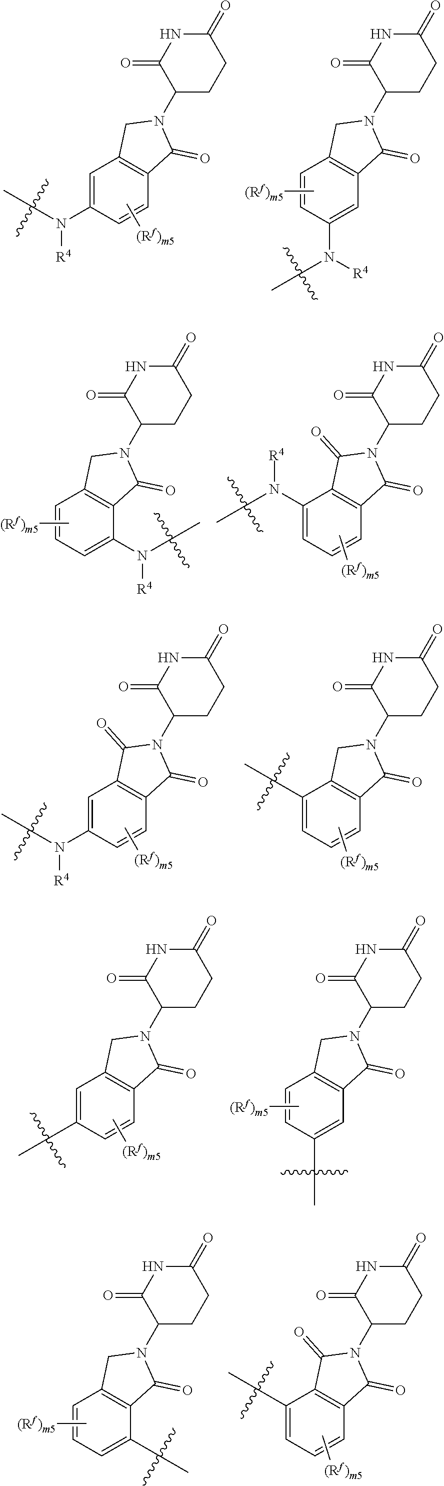

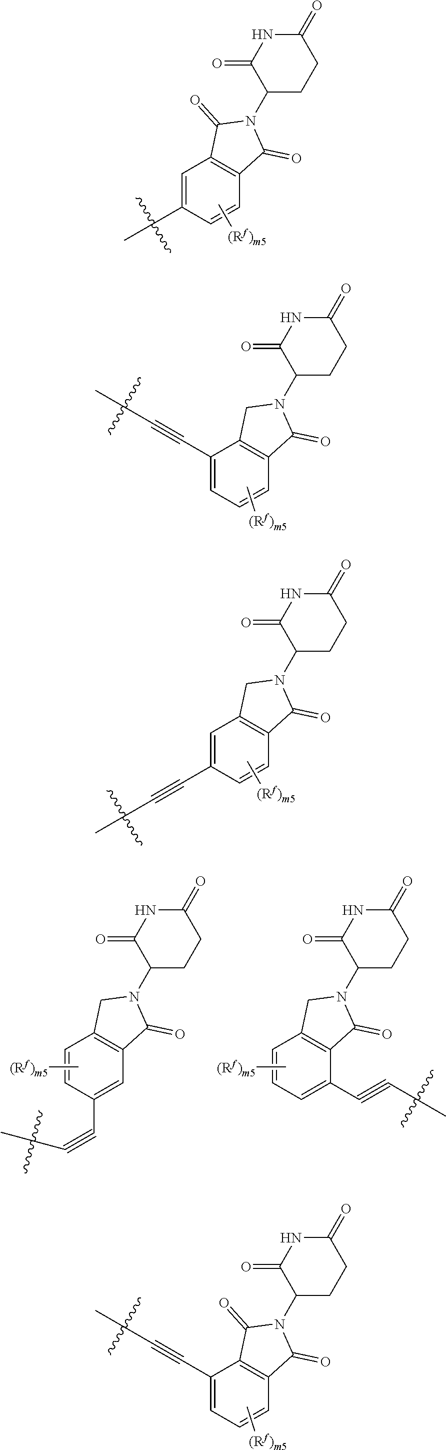

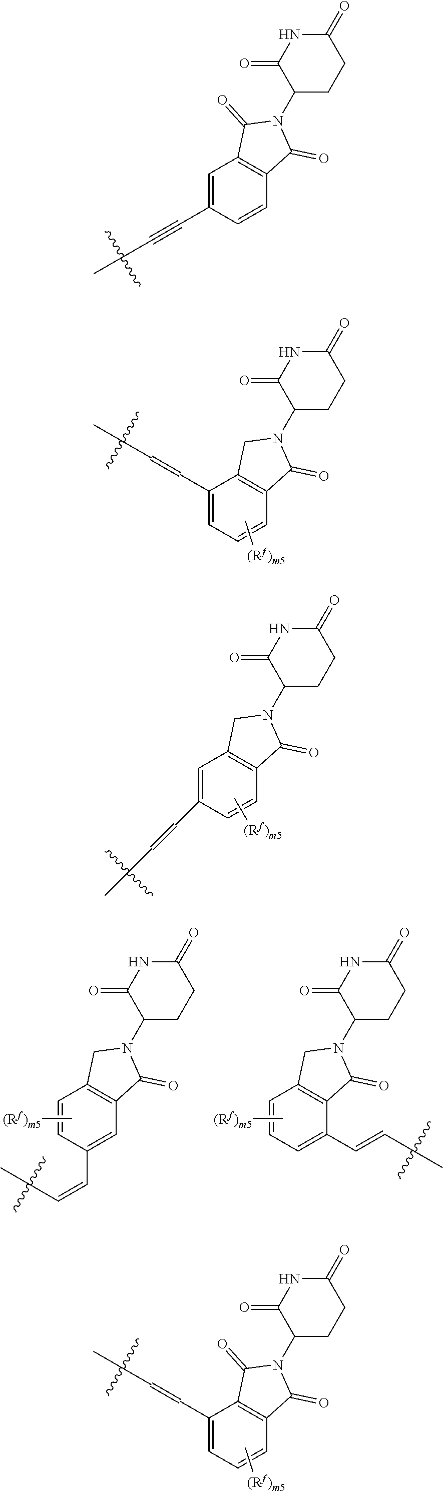

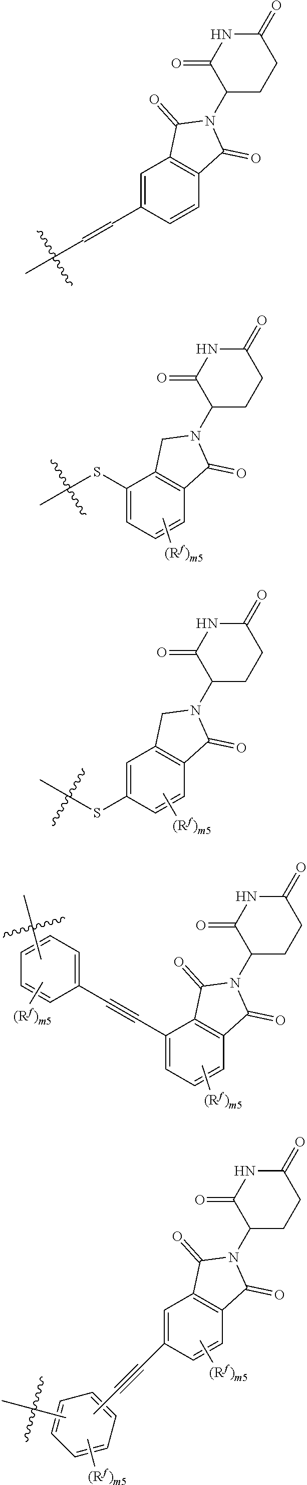

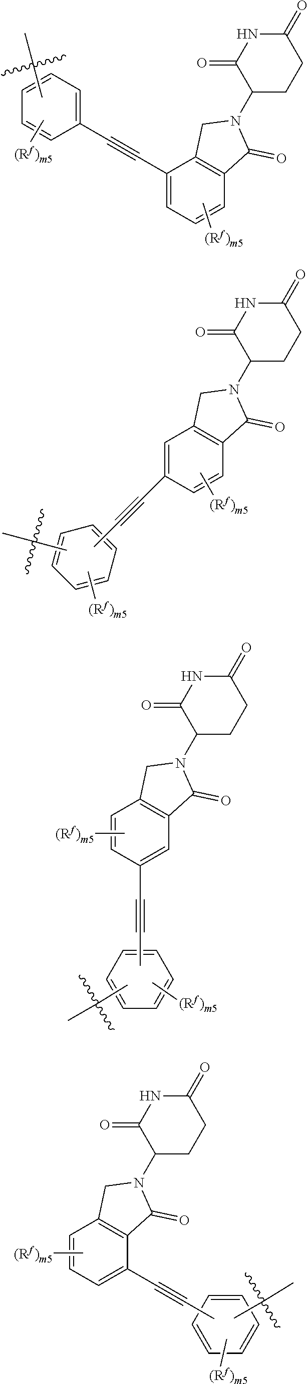

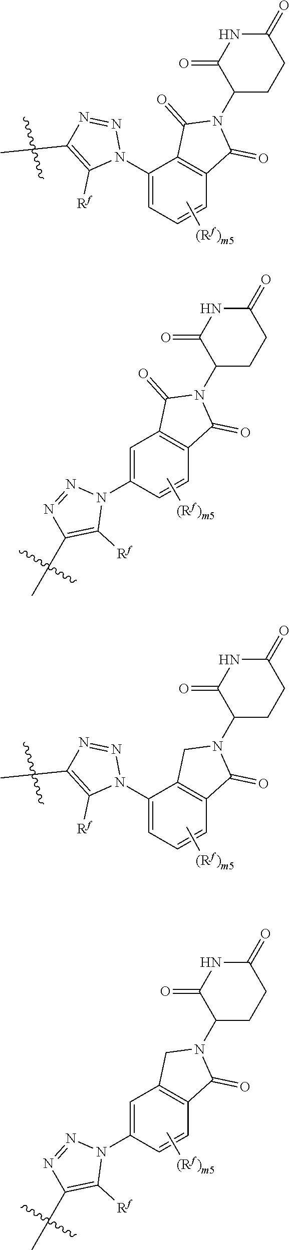

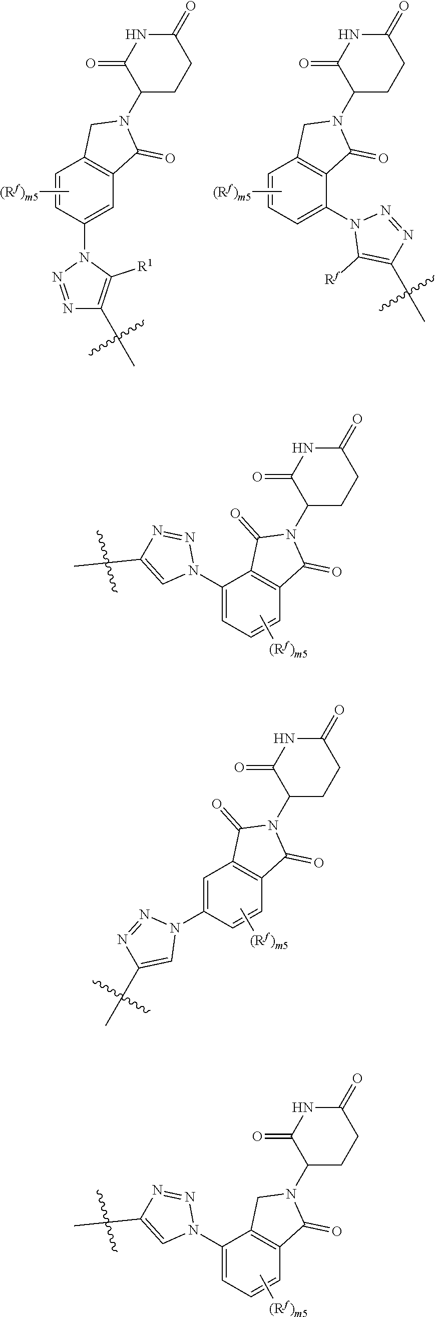

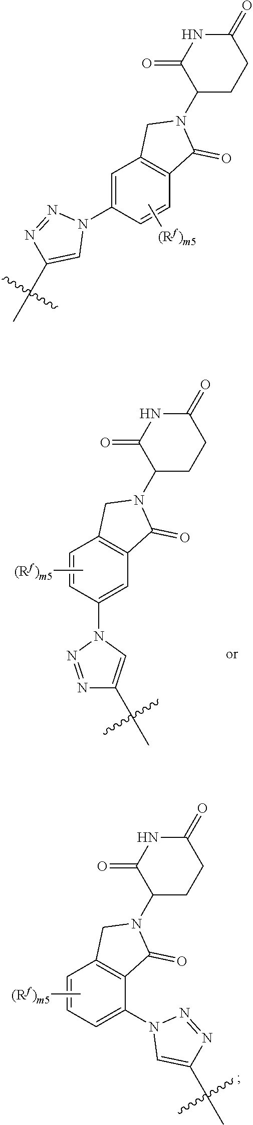

wherein R⁴ represents H or C₁₋₃ alkyl (e.g., methyl, ethyl, or propyl); (R^{f})ₘ₅ indicates that phenyl ring is optionally substituted by m5 R^{f} groups, where each R^{f} is independently halogen (e.g., fluorine, chlorine, bromine, or iodine), and m5 represents an integer of 0, 1, 2, or 3.

In some embodiments, ULM represents a structure of Formula (IV):

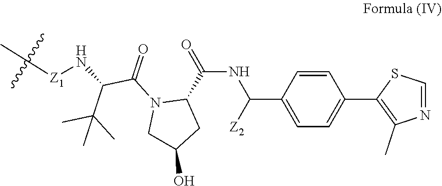

wherein Z₁ represents C(O) or Z₁ represents a bond, and Z₂ represents H or CH₃.

In some embodiments, ULM represents the structure of the following formula:

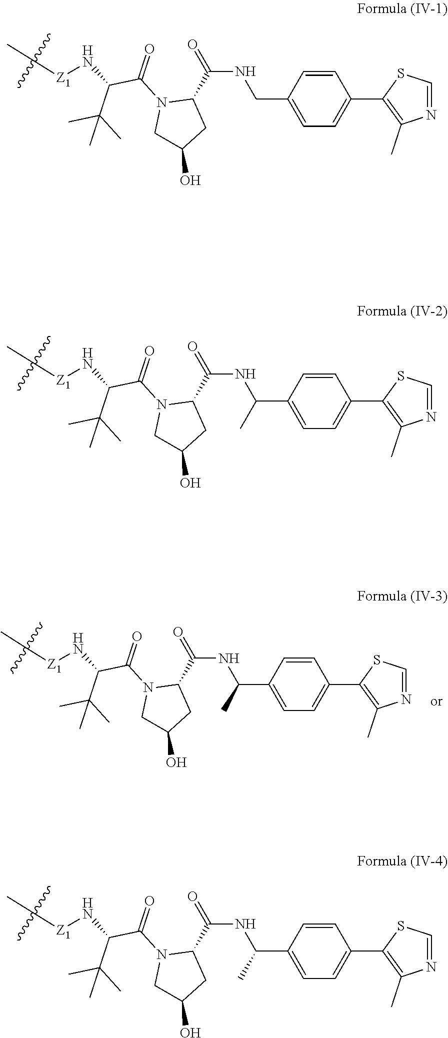

wherein Z₁ represents C(O) or Z₁ represents a bond.

In some embodiments, LIN is represented by the following formula:

#-U-alkylene,
wherein U represents C(O) or U represents a bond, and the symbol #indicates the point of attachment to Y; and
the alkylene is substituted or unsubstituted methylene, or substituted or unsubstituted linear or branched C₂₋₆₀ alkylene, wherein one or more groups R⁵ and/or one or more groups R⁶ and/or any combination of one or more groups R⁵ and R⁶ are optionally inserted into the backbone carbon chain of the linear or branched C₂₋₆₀ alkylene group. In other words, when one or more groups R⁵ and/or one or more groups R⁶ and/or any combination of one or more groups R⁵ and R⁶ are optionally inserted into the backbone carbon chain of the linear or branched C₂₋₆₀ alkylene group, carbon-carbon bond between one or more pairs of adjacent carbon atoms in the backbone carbon chain is interrupted by the group R⁵, R⁶, or a combination of R⁵ and R⁶. In some embodiments, each R⁵ is independently selected from the group consisting of O, N(R⁷), C(O), C(O)O, OC(O), C(O)N(R⁷), N(R⁷)C(O), or N(R⁷)C(O)N(R⁷), where each R⁷ independently represents H or C₁₋₃ alkyl (e.g., methyl, ethyl, or propyl), and in case that two or more groups R⁵ are inserted into the backbone carbon chain of the linear or branched C₂₋₆₀ alkylene group, the two or more groups R⁵ are not directly connected to each other. In some embodiments, each R⁶ is independently selected from the group consisting of cycloalkylene, arylene, heterocyclylene, heteroarylene, alkynylene (e.g., ethynylene

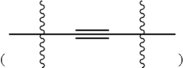 [SMILES: *C#C*]

or butynylene

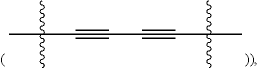 [SMILES: *C#CC#C*]

alkenylene (e.g., vinylene

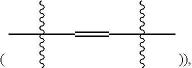 [SMILES: *C=C*]

or any combination thereof, wherein the cycloalkylene, arylene, heterocyclylene, and heteroarylene are each independently optionally substituted by a substituent selected from the group consisting of C₁₋₃ alkyl (e.g., methyl, ethyl, or propyl), C₃₋₆ cycloalkyl (e.g., cyclopropyl, cyclobutyl, cyclopentyl, or cyclohexyl), hydroxyl, amino, mercapto, halogen (e.g., fluorine, chlorine, bromine, or iodine), C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), halogenated C₁₋₃ alkyl (e.g., —CF₃, —CH₂F, —CHF₂, —CH₂Cl, —CHCl₂, —CF₂CF₃, —CHFCF₃, —CF₂CHF₂, —CHFCHF₂, —CH₂CF₃, and —CH₂CH₂Cl), amino-substituted C₁₋₃ alkylene (NH₂—C₁₋₃ alkylene-, e.g., NH₂CH₂—, NH₂CH₂CH₂—, and NH₂CH₂CH₂CH₂—), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), cyano, or any combination thereof. In some sub-embodiments, the backbone carbon chain of the alkylene (e.g., substituted or unsubstituted linear or branched C₂₋₆₀ alkylene group) optionally contains one or more (e.g., 1-30, 1-20, 1-15, 1-10, 1-8, 1-7, 1-6, 1-5, 1-4, 1-3, 1-2, or 1) fragments “—CH₂—R⁵—CH₂—” and/or one or more (e.g., 1-30, 1-20, 1-15, 1-10, 1-8, 1-7, 1-6, 1-5, 1-4, 1-3, 1-2, or 1) fragments “—CH₂—R⁶—CH₂—” and/or one or more (e.g., 1-30, 1-20, 1-15, 1-10, 1-8, 1-7, 1-6, 1-5, 1-4, 1-3, 1-2, or 1) fragments “—CH₂—R⁵—R⁶—CH₂—”, wherein each R⁵ are the same or different, each R⁶ are the same or different, and are as defined herein.

In some embodiments, LIN is represented by the following formula:

#-U-alkylene,
wherein U represents C(O) or U represents a bond, and the symbol #indicates the point of attachment to Y; and
the alkylene is substituted or unsubstituted linear or branched C₁₋₆₀ alkylene (e.g., C₁₋₅₅ alkylene, C₁₋₅₀ alkylene, C₁₋₄₅ alkylene, C₁₋₄₀ alkylene, C₁₋₃₅ alkylene, C₁₋₃₀ alkylene, C₁₋₂₅ alkylene, C₁₋₂₃ alkylene, C₁₋₂₂ alkylene, C₁₋₂₁ alkylene, C₁₋₂₀ alkylene, C₂₋₂₀ alkylene, C₁₋₁₉ alkylene, C₂₋₁₉ alkylene, C₁₋₁₈ alkylene, C₂₋₁₈ alkylene, C₁₋₁₇ alkylene, C₂₋₁₇ alkylene, C₁₋₁₆ alkylene, C₂₋₁₆ alkylene, C₁₋₁₅ alkylene, C₂₋₁₅ alkylene, C₁₋₁₄ alkylene, C₂₋₁₄ alkylene, C₁₋₁₃ alkylene, C₂₋₁₃ alkylene, C₁₋₁₂ alkylene, C₂₋₁₂ alkylene, C₁₋₁₁ alkylene, C₂₋₁₁ alkylene, C₁₋₁₀ alkylene, C₂₋₁₀ alkylene, C₃₋₁₀ alkylene, C₁₋₉ alkylene, C₂₋₉ alkylene, C₃₋₉ alkylene, C₁₋₈ alkylene, C₂₋₈ alkylene, C₃₋₈ alkylene, C₁₋₇ alkylene, C₂₋₇ alkylene, C₃₋₇ alkylene, C₁₋₆ alkylene, C₂₋₆ alkylene, C₃₋₆ alkylene, C₁₋₅ alkylene, C₂₋₅ alkylene, C₃₋₅ alkylene, C₁₋₄ alkylene, C₁₋₃ alkylene, C₁₋₂ alkylene, methylene). When the alkylene is substituted linear or branched C₁₋₆₀ alkylene, exemplary examples of substituents are optionally selected from the group consisting of C₁₋₃ alkyl (e.g., methyl, ethyl, or propyl), C₃₋₆ cycloalkyl (e.g., cyclopropyl, cyclobutyl, cyclopentyl, or cyclohexyl), hydroxyl, amino, mercapto, halogen (e.g., fluorine, chlorine, bromine, or iodine), C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), halogenated C₁₋₃ alkyl (e.g., F₃C—, FCH₂—, F₂CH—, ClCH₂—, Cl₂CH—, CF₃CF₂—, CF₃CHF—, CHF₂CF₂—, CHF₂CHF—, CF₃CH₂—, and CH₂ClCH₂—), amino-substituted C₁₋₃ alkylene (NH₂—C₁₋₃ alkylene-, e.g., NH₂CH₂—, NH₂CH₂CH₂—, and NH₂CH₂CH₂CH₂—), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), cyano, or any combination thereof.

In some embodiments, the alkylene in LIN can be substituted linear or branched C₁₋₆₀ alkylene, e.g., linear or branched C₁₋₆₀ alkylene (e.g., C₁₋₅₅ alkylene, C₁₋₅₀ alkylene, C₁₋₄₅ alkylene, C₁₋₄₀ alkylene, C₁₋₃₅ alkylene, C₁₋₃₀ alkylene, C₁₋₂₅ alkylene, C₁₋₂₃ alkylene, C₁₋₂₂ alkylene, C₁₋₂₁ alkylene, C₁₋₂₀ alkylene, C₂₋₂₀ alkylene, C₁₋₁₉ alkylene, C₂₋₁₉ alkylene, C₁₋₁₈ alkylene, C₂₋₁₈ alkylene, C₁₋₁₇ alkylene, C₂₋₁₇ alkylene, C₁₋₁₆ alkylene, C₂₋₁₆ alkylene, C₁₋₁₅ alkylene, C₂₋₁₅ alkylene, C₁₋₁₄ alkylene, C₂₋₁₄ alkylene, C₁₋₁₃ alkylene, C₂₋₁₃ alkylene, C₁₋₁₂ alkylene, C₂₋₁₂ alkylene, C₁₋₁₁ alkylene, C₂₋₁₁ alkylene, C₁₋₁₀ alkylene, C₂₋₁₀ alkylene, C₃₋₁₀ alkylene, C₁₋₉ alkylene, C₂₋₉ alkylene, C₃₋₉ alkylene, C₁₋₈ alkylene, C₂₋₈ alkylene, C₃₋₈ alkylene, C₁₋₇ alkylene, C₂₋₇ alkylene, C₃₋₇ alkylene, C₁₋₆ alkylene, C₂₋₆ alkylene, C₃₋₆ alkylene, C₁₋₅ alkylene, C₂₋₅ alkylene, C₃₋₅ alkylene, C₁₋₄ alkylene, C₁₋₃ alkylene, C₁₋₂ alkylene, methylene) substituted by one or more identical or different substituents, wherein the substituents are optionally selected from the group consisting of C₁₋₃ alkyl (e.g., methyl, ethyl, or propyl), C₃₋₆ cycloalkyl (e.g., cyclopropyl, cyclobutyl, cyclopentyl, or cyclohexyl), hydroxyl, amino, mercapto, halogen (e.g., fluorine, chlorine, bromine, or iodine), C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), halogenated C₁₋₃ alkyl (e.g., F₃C—, FCH₂—, F₂CH—, ClCH₂—, Cl₂CH—, CF₃CF₂—, CF₃CHF—, CHF₂CF₂—, CHF₂CHF—, CF₃CH₂—, and CH₂ClCH₂—), amino-substituted C₁₋₃ alkylene (NH₂—C₁₋₃ alkylene-, e.g., NH₂CH₂—, NH₂CH₂CH₂—, and NH₂CH₂CH₂CH₂—), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), cyano, or any combination thereof. In a sub-embodiment of the present disclosure, the linear or branched C₁₋₆₀ alkylene is optionally substituted by one or more (e.g., 1-30, 1-25, 1-20, or 1-15, 1-10, 1-9, 1-8, 1-7, 1-6, 1-5, 1-4, 1-3, or 1-2; or 20, 19, 18, 17, 16, 15, 14, 13, 12, 11, 10, 9, 8, 7, 6, 5, 4, 3, 2, or 1) identical or different substituents. The number of substituents is in principle not limited in any way, or is automatically limited by the size of the building unit.

In some embodiments, LIN represents the following groups: #—U—CH₂—, #-U—(CH₂)₂—, #-U—(CH₂)₃—, #-U—(CH₂)₄—, #-U—(CH₂)₅—, #-U—(CH₂)₆—, #-U—(CH₂)₇—, #-U—(CH₂)₈—, #-U—(CH₂)₉—, #-U—(CH₂)₁₀—, #-U—(CH₂)₁₁—, #-U—(CH₂)₁₂—, #-U—(CH₂)₁₃—, #-U—(CH₂)₁₄—, #-U—(CH₂)₁₅—, #-U—(CH₂)₁₆—, #-U—(CH₂)₁₇—, #-U—(CH₂)₁₈—, #-U—(CH₂)₁₉—, #-U—(CH₂)₂₀—, #-U—(CH₂)₂₁—, #-U—(CH₂)₂₂—, #-U—(CH₂)₂₅—, #-U—(CH₂)₃₀—, #-U—(CH₂)₃₅—, #-U—(CH₂)₄₀—, #-U—(CH₂)₄₅—, #-U—(CH₂)₅₀—, #-U—(CH₂)₅₅—, or #- U—(CH₂)₆₀—; wherein a hydrogen atom of one or more CH₂ groups of the groups is optionally replaced with a substituent selected from the group consisting of C₁₋₃ alkyl (e.g., methyl, ethyl, or propyl), C₃₋₆ cycloalkyl (e.g., cyclopropyl, cyclobutyl, cyclopentyl, or cyclohexyl), hydroxyl, amino, mercapto, halogen (e.g., fluorine, chlorine, bromine, or iodine), C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), halogenated C₁₋₃ alkyl (e.g., F₃C—, FCH₂—, F₂CH—, ClCH₂—, Cl₂CH—, CF₃CF₂—, CF₃CHF—, CHF₂CF₂—, CHF₂CHF—, CF₃CH₂—, and CH₂ClCH₂—), amino-substituted C₁₋₃ alkylene (NH₂—C₁₋₃ alkylene-, e.g., NH₂CH₂—, NH₂CH₂CH₂—, and NH₂CH₂CH₂CH₂—), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), cyano, or any combination thereof; and U represents C(O) or U represents a bond, and the symbol #indicates the point of attachment to Y.

In some embodiments, LIN represents is represented by the following formula:

#-U-alkylene,
wherein U represents C(O) or U represents a bond, and the symbol #indicates the point of attachment to Y; and
the alkylene is substituted or unsubstituted linear or branched C₂₋₆₀ alkylene, wherein one or more groups R⁵ and/or one or more groups R⁶ and/or any combination of one or more groups R⁵ and R⁶ are inserted into the backbone carbon chain of the linear or branched C₂₋₆₀ alkylene group. In other words, when one or more groups R⁵ and/or one or more groups R⁶ and/or any combination of one or more groups R⁵ and R⁶ are inserted into the backbone carbon chain of the linear or branched C₂₋₆₀ alkylene group, carbon-carbon bond between one or more pairs of adjacent carbon atoms in the backbone carbon chain is interrupted by the group R⁵, R⁶, or any combination of R⁵ and R⁶. In some embodiments, each R⁵ is independently selected from the group consisting of O, N(R⁷), C(O), C(O)O, OC(O), C(O)N(R⁷), N(R⁷)C(O), or N(R⁷)C(O)N(R⁷), where each R⁷ independently represents H or C₁₋₃ alkyl (e.g., methyl, ethyl, or propyl), and in case that two or more groups R⁵ are inserted into the backbone carbon chain of the linear or branched C₂₋₆₀ alkylene group, the two or more groups R⁵ are not directly connected to each other. In some embodiments, each R⁶ is independently selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted by one or more (e.g., 1-4, 1-3, 1-2, or 1) substituents selected from the group consisting of C₁₋₃ alkyl (e.g., methyl, ethyl, or propyl), C₃₋₆ cycloalkyl (e.g., cyclopropyl, cyclobutyl, cyclopentyl, or cyclohexyl), hydroxyl, amino, mercapto, halogen (e.g., fluorine, chlorine, bromine, or iodine), C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), halogenated C₁₋₃ alkyl (e.g., —CF₃, —CH₂F, —CHF₂, —CH₂Cl, —CHCl₂, —CF₂CF₃, —CHFCF₃, —CF₂CHF₂, —CHFCHF₂, —CH₂CF₃, and —CH₂CH₂Cl), amino-substituted C₁₋₃ alkylene (NH₂—C₁₋₃ alkylene-, e.g., NH₂CH₂—, NH₂CH₂CH₂—, and NH₂CH₂CH₂CH₂—), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), cyano, or any combination thereof. In some embodiments, the number of groups R⁵, the number of R⁶, or the number of any combination of R⁵ and R⁶ may be independently, for example, 1-30, 1-20, 1-15, 1-10, 1-8, 1-7, 1-6, 1-5, 1-4, 1-3, 1-2, or 1, respectively. The number of R⁵, R⁶, or any combination of R⁵ and R⁶ are in principle not limited in any way, or are automatically limited by the size of the building unit. In some sub-embodiments, the backbone carbon chain of the linear or branched C₂₋₆₀ alkylene optionally contains one or more (e.g., 1-30, 1-20, 1-15, 1-10, 1-8, 1-7, 1-6, 1-5, 1-4, 1-3, 1-2, or 1) fragments “—CH₂—R⁵—CH₂—” and/or one or more (e.g., 1-30, 1-20, 1-15, 1-10, 1-8, 1-7, 1-6, 1-5, 1-4, 1-3, 1-2, or 1) fragments “—CH₂—R⁶—CH₂—” and/or one or more (e.g., 1-30, 1-20, 1-15, 1-10, 1-8, 1-7, 1-6, 1-5, 1-4, 1-3, 1-2, or 1) fragments “—CH₂—R⁵—R⁶—CH₂—”, wherein each R⁵ are the same or different, each R⁶ are the same or different, and are as defined herein.

Herein, when one or more groups R⁵ and/or one or more groups R⁶ and/or one or more any combination of R⁵ and R⁶ are inserted into the backbone carbon chain of the linear or branched C₂₋₆₀ alkylene group, the resulting backbone chain group conforms to the covalent bond theory, and can contain one or more (e.g., 1-30, 1-20, 1-15, 1-10, 1-8, 1-7, 1-6, 1-5, 1-4, 1-3, 1-2, or 1) fragments “—CH₂—R⁵—CH₂—” and/or one or more (e.g., 1-30, 1-20, 1-15, 1-10, 1-8, 1-7, 1-6, 1-5, 1-4, 1-3, 1-2, or 1) fragments “—CH₂—R⁶—CH₂—” and/or one or more (e.g., 1-30, 1-20, 1-15, 1-10, 1-8, 1-7, 1-6, 1-5, 1-4, 1-3, 1-2, or 1) fragments “—CH₂—R⁵—R⁶—CH₂—”, wherein each R⁵ are the same or different, each R⁶ are the same or different, and are as defined herein.

In some embodiments, LIN can be represented by the following formula:

#-U—(C(R^{a1})(R^{a2}))ₙ₄—(R⁵(C(R^{a3})(R^{a4}))ₙ₅)ₘ₆—;

#-U—(C(R^{a1})(R^{a2}))ₙ₄—(R⁵(C(R^{a3})(R^{a4}))ₙ₅)ₘ₆—(R⁵(C(R^{a5})(R^{a6}))ₙ₆)ₘ₇—;

#-U—(C(R^{a1})(R^{a2}))ₙ₄—(R⁵(C(R^{a3})(R^{a4}))ₙ₅)ₘ₆—(R⁵(C(R^{a5})(R^{a6}))ₙ₆)ₘ₇—(R⁵(C(R^{a7})(R^{a8}))ₙ₇)ₘ₈—;

#-U—(C(R^{a1})(R^{a2}))ₙ₄—(R⁶(C(R^{a3})(R^{a4}))ₙ₅)ₘ₆—;

#-U—(C(R^{a1})(R^{a2}))ₙ₄—(R⁶(C(R^{a3})(R^{a4}))ₙ₅)ₘ₆—(R⁶(C(R^{a5})(R^{a6}))ₙ₆)ₘ₇—;

#-U—(C(R^{a1})(R^{a2}))ₙ₄—(R⁶(C(R^{a3})(R^{a4}))ₙ₅)ₘ₆—(R⁶(C(R^{a5})(R^{a6}))ₙ₆)ₘ₇—(R⁶(C(R^{a7})(R^{a5}))ₙ₇)ₘ₈—;

#-U—(C(R^{a1})(R^{a2}))ₙ₄—(R⁵-R⁶—(CR^{a3})(R^{a4}))ₙ₅)ₘ₆—;

#-U—(C(R^{a1})(R^{a2}))ₙ₄—(R⁵(C(R^{a3})(R^{a4}))ₙ₅)ₘ₆—(R⁶(CR^{a5})(R^{a6}))ₙ₆)ₘ₇—;

#-U—(C(R^{a1})(R^{a2}))ₙ₄—(R⁶—R⁵—(C(R^{a3})(R^{a4}))ₙ₅)ₘ₆—; or

#-U—(C(R^{a1})(R^{a2}))ₙ₄—(R⁶(C(R^{a3})(R^{a4}))ₙ₅)ₘ₆—(R⁵(C(R^{a5})(R^{a6}))ₙ₆)ₘ₇—;
wherein each R⁵ is independently selected from the group consisting of O, N(R⁷), C(O), C(O)O, OC(O), C(O)N(R⁷), N(R⁷)C(O), or N(R⁷)C(O)N(R⁷), where each R⁷ independently represents H or C₁₋₃ alkyl (e.g., methyl, ethyl, or propyl), and in case that two or more groups R⁵ are inserted into the backbone carbon chain of the linear or branched C₂₋₆₀ alkylene group, the two or more groups R⁵ are not directly connected to each other;
wherein each R⁶ is independently selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted by one or more (e.g., 1-4, 1-3, 1-2, or 1) substituents selected from the group consisting of C₁₋₃ alkyl (e.g., methyl, ethyl, or propyl), C₃₋₆ cycloalkyl (e.g., cyclopropyl, cyclobutyl, cyclopentyl, or cyclohexyl), hydroxyl, amino, mercapto, halogen (e.g., fluorine, chlorine, bromine, or iodine), C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), halogenated C₁₋₃ alkyl (e.g., F₃C—, FCH₂—, F₂CH—, ClCH₂—, Cl₂CH—, CF₃CF₂—, CF₃CHF—, CHF₂CF₂—, CHF₂CHF—, CF₃CH₂—, and CH₂ClCH₂—), amino-substituted C₁₋₃ alkylene (NH₂—C₁₋₃ alkylene-, e.g., NH₂CH₂—, NH₂CH₂CH₂—, and NH₂CH₂CH₂CH₂—), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), cyano, or any combination thereof;
R^{a1}, R^{a2}, R^{a3}, R^{a4}, R^{a5}, R^{a6}, R^{a7}, and R^{a8} each independently represent H, C₁₋₃ alkyl (e.g., methyl, ethyl, or propyl), C₃₋₆ cycloalkyl (e.g., cyclopropyl, cyclobutyl, cyclopentyl, or cyclohexyl), hydroxyl, amino, mercapto, halogen (e.g., fluorine, chlorine, bromine, or iodine), C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), amino-substituted C₁₋₃ alkylene (NH₂—C₁₋₃ alkylene-, e.g., NH₂CH₂—, NH₂CH₂CH₂—, and NH₂CH₂CH₂CH₂—), halogenated C₁₋₃ alkyl (e.g., F₃C—, FCH₂—, F₂CH—, ClCH₂—, Cl₂CH—, CF₃CF₂—, CF₃CHF—, CHF₂CF₂—, CHF₂CHF—, CF₃CH₂—, and CH₂ClCH₂—), or cyano,
U represents C(O) or U represents a bond, and the symbol #indicates the point of attachment to Y; and
n4, n5, n6, n7, m6, m7, m8 each independently represent an integer of 1, 2, 3, 4, 5, 6, 7, 8, 9, 10, 11, 12, 13, 14, 15, 16, 17, 18, 19, or 20.

In some embodiments, LIN can be represented by the following formula:

#-U—(C(R^{a1})(R^{a2}))ₙ₄—(O(C(R^{a3})(R^{a4}))ₙ₅)ₘ₆—;

#-U—(C(R^{a1})(R^{a2}))ₙ₄—(O(C(R^{a3})(R^{a4}))ₙ₅)ₘ₆—(O(C(R^{a5})(R^{a6}))ₙ₆)ₘ₇—;

#-U—(C(R^{a1})(R^{a2}))ₙ₄—(O(C(R^{a3})(R^{a4}))ₙ₅)ₘ₆—(O(C(R^{a5})(R^{a6}))ₙ₆)ₘ₇—(O(C(R^{a7})(R^{a8}))ₙ₇)ₘ₈—;

#-U—(C(R^{a1})(R^{a2}))ₙ₄—(N(R⁷)(C(R^{a3})(R^{a4}))ₙ₅)ₘ₆—;

#-U—(C(R^{a1})(R^{a2}))ₙ₄—(N(R⁷)(C(R^{a3})(R^{a4}))ₙ₅)ₘ₆—(N(R⁷)(C(R^{a5})(R^{a6}))ₙ₆)ₘ₇—;

#-U—(C(R^{a1})(R^{a2}))ₙ₄—(N(R⁷)(C(R^{a3})(R^{a4}))ₙ₅)ₘ₆—(N(R⁷)(C(R^{a5})(R^{a6}))ₙ₆)ₘ₇—(N(R⁷)(C(R^{a7})(R^{a8}))ₙ₇)ₘ₈;

#-U—(C(R^{a1})(R^{a2}))ₙ₄—(C(O)N(R⁷)—(C(R^{a3})(R^{a4}))ₙ₅)ₘ₆—;

#-U—(C(R^{a1})(R^{a2}))ₙ₄—(C(O)N(R⁷)—(C(R^{a3})(R^{a4}))ₙ₅)ₘ₆—(C(O)N(R⁷)—(C(R^{a5})(R^{a6}))ₙ₆)ₘ₇—;

#-U—(C(R^{a1})(R^{a2}))ₙ₄—(C(O)N(R⁷)—(C(R^{a3})(R^{a4}))ₙ₅)ₘ₆—(C(O)N(R⁷)—(C(R^{a5})(R^{a6}))ₙ₆)ₘ₇—(C(O)N(R⁷)—(C(R^{a7})(R^{a8}))ₙ₇)ₘ₈—;

#-U—(C(R^{a1})(R^{a2}))ₙ₄—(C(O)N(R⁷)—(C((R^{a3})(R^{a4}))ₙ₅)ₘ₆—(O—(C(R^{a5})(R^{a6}))ₙ₆)ₘ₇—;

#-U—(C(R^{a1})(R^{a2}))ₙ₄—C(O)N(R⁷)—(C(R^{a3})(R^{a4}))ₙ₅—(O(C(R^{a5})(R^{a6}))ₙ₆)ₘ₆—;

#-U—(C(R^{a1})(R^{a2}))ₙ₄—(N(R⁷)C(O)—(C(R^{a3})(R^{a4}))ₙ₅)ₘ₆—;

#-U—(C(R^{a1})(R^{a2}))ₙ₄—(N(R⁷)C(O)—(C(R^{a3})(R^{a4}))ₙ₅)ₘ₆—(O(C(R^{a5})(R^{a6}))ₙ₆)ₘ₇;

#-U—(C(R^{a1})(R^{a2}))ₙ₄—(N(R⁷)C(O)—(C(R^{a3})(R^{a4}))ₙ₅)ₘ₆—(O—(C(R^{a5})(R^{a6}))ₙ₆)ₘ₇—(O—(C(R^{a7})(R^{a8}))ₙ₇)ₘ₈—;

#-U—(C(R^{a1})(R^{a2}))ₙ₄—N(R⁷)C(O)—(C(R^{a3})(R^{a4}))ₙ₅—(O(C(R^{a5})(R^{a6}))ₙ₆)ₘ₆—;

#-U—(C(R^{a1})(R^{a2}))ₙ₄—N(R⁷)C(O)N(R⁷)—(C(R^{a3})(R^{a4}))ₙ₅—;

#-U—(C(R^{a1})(R^{a2}))ₙ₄—C(O)—(C(R^{a3})(R^{a4}))ₙ₅—;

#-U—(C(R^{a1})(R^{a2}))ₙ₄—CH═CH—(C(R^{a3})(R^{a4}))ₙ₅—;

#-U—(C(R^{a1})(R^{a2}))ₙ₄—C≡C—(C(R^{a3})(R^{a4}))ₙ₅—;

#-U—(C(R^{a1})(R^{a2}))ₙ₄—C≡C—C≡C—(C(R^{a3})(R^{a4}))ₙ₅—;

#-U—(C(R^{a1})(R^{a2}))ₙ₄-(arylene-(C(R^{a3})(R^{a4}))ₙ₅)ₘ₆—;

#-U—(C(R^{a1})(R^{a2}))ₙ₄-(arylene-(C(R^{a3})(R^{a4}))ₙ₅)ₘ₆-arylene-(C(R^{a5})(R^{a6}))ₙ₆—;

#-U—(C(R^{a1})(R^{a2}))ₙ₄-(heterocyclylene-(C(R^{a3})(R^{a4}))ₙ₅)ₘ₆—;

#-U—(C(R^{a1})(R^{a2}))ₙ₄-(heterocyclylene-(C(R^{a3})(R^{a4}))ₙ₅)ₘ₆-(heterocyclylene-(C(R^{a5})(R^{a6}))ₙ₆)ₘ₇—;

#-U—(C(R^{a1})(R^{a2}))ₙ₄-(heteroarylene-(C(R^{a3})(R^{a4}))ₙ₅)ₘ₆—;

#-U—(C(R^{a1})(R^{a2}))ₙ₄-(heteroarylene-(C(R^{a3})(R^{a4}))ₙ₅)ₘ₆-(heteroarylene-(C(R^{a5})(R^{a6}))ₙ₆)ₘ₇—;

#-U—(C(R^{a1})(R^{a2}))ₙ₄-(cycloalkylene-(C(R^{a3})(R^{a4}))ₙ₅)ₘ₆—; or

#-U—(C(R^{a1})(R^{a2}))ₙ₄-(cycloalkylene-(C(R^{a3})(R^{a4}))ₙ₅)ₘ₆-(cycloalkylene-(C(R^{a5})(R^{a6}))ₙ₆)ₘ₇—;
wherein the cycloalkylene, arylene, heterocyclylene, and heteroarylene are each independently optionally substituted by one or more (e.g., 1-4, 1-3, 1-2, or 1) substituents selected from the group consisting of C₁₋₃ alkyl (e.g., methyl, ethyl, or propyl), C₃₋₆ cycloalkyl (e.g., cyclopropyl, cyclobutyl, cyclopentyl, or cyclohexyl), hydroxyl, amino, mercapto, halogen (e.g., fluorine, chlorine, bromine, or iodine), C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), halogenated C₁₋₃ alkyl (e.g., F₃C—, FCH₂—, F₂CH—, ClCH₂—, Cl₂CH—, CF₃CF₂—, CF₃CHF—, CHF₂CF₂—, CHF₂CHF—, CF₃CH₂—, and CH₂ClCH₂—), amino-substituted C₁₋₃ alkylene (NH₂—C₁₋₃ alkylene-, e.g., NH₂CH₂—, NH₂CH₂CH₂—, and NH₂CH₂CH₂CH₂—), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH—(e.g., CH₃—C(O)NH—, CH₃CH₂—C(O)NH—, and CH₃CH₂CH₂—C(O)NH—), cyano, or any combination thereof;
each R⁷ independently represents H or C₁₋₃ alkyl (e.g., methyl, ethyl, or propyl);
R^{a1}, R^{a2}, R^{a3}, R^{a4}, R^{a5}, R^{a6}, R^{a7}, and R^{a8} each independently represent H, C₁₋₃ alkyl (e.g., methyl, ethyl, or propyl), C₃₋₆ cycloalkyl (e.g., cyclopropyl, cyclobutyl, cyclopentyl, or cyclohexyl), hydroxyl, amino, mercapto, halogen (e.g., fluorine, chlorine, bromine, or iodine), C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), amino-substituted C₁₋₃ alkylene (NH₂—C₁₋₃ alkylene-, e.g., NH₂CH₂—, NH₂CH₂CH₂—, and NH₂CH₂CH₂CH₂—), halogenated C₁₋₃ alkyl (e.g., F₃C—, FCH₂—, F₂CH—, ClCH₂—, Cl₂CH—, CF₃CF₂—, CF₃CHF—, CHF₂CF₂—, CHF₂CHF—, CF₃CH₂—, and CH₂ClCH₂—), or cyano;
U represents C(O) or U represents a bond, and the symbol #indicates the point of attachment to Y; and
n4, n5, n6, n7, n8, m6, m7, m8 each independently represent an integer of 1, 2, 3, 4, 5, 6, 7, 8, 9, 10, 11, 12, 13, 14, 15, 16, 17, 18, 19, or 20.

In some embodiments, the cycloalkylene in LIN is optionally selected from the group consisting of:
cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, C₅₋₁₅ spiro-cycloalkylene, adamantanylene, noradamantanylene, bornylene, bicyclo[2.2.1]heptanylene, or bicyclo[2.2.1]heptenylene;
wherein the cycloalkylene is optionally substituted by one or more (e.g., 1-4, 1-3, 1-2, or 1) substituents selected from the group consisting of C₁₋₃ alkyl (e.g., methyl, ethyl, or propyl), C₃₋₆ cycloalkyl (e.g., cyclopropyl, cyclobutyl, cyclopentyl, or cyclohexyl), hydroxyl, amino, mercapto, halogen (e.g., fluorine, chlorine, bromine, or iodine), C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), halogenated C₁₋₃ alkyl (e.g., F₃C—, FCH₂—, F₂CH—, ClCH₂—, Cl₂CH—, CF₃CF₂—, CF₃CHF—, CHF₂CF₂—, CHF₂CHF—, CF₃CH₂—, and CH₂ClCH₂—), amino-substituted C₁₋₃ alkylene (NH₂—C₁₋₃ alkylene-, e.g., NH₂CH₂—, NH₂CH₂CH₂—, and NH₂CH₂CH₂CH₂—), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), cyano, or any combination thereof.

In some embodiments, the arylene in LIN is optionally selected from the group consisting of: phenylene or naphthylene, wherein the arylene is optionally substituted by one or more (e.g., 1-4, 1-3, 1-2, or 1) substituents selected from the group consisting of C₁₋₃ alkyl (e.g., methyl, ethyl, or propyl), C₃₋₆ cycloalkyl (e.g., cyclopropyl, cyclobutyl, cyclopentyl, or cyclohexyl), hydroxyl, amino, mercapto, halogen (e.g., fluorine, chlorine, bromine, or iodine), C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), halogenated C₁₋₃ alkyl (e.g., F₃C—, FCH₂—, F₂CH—, ClCH₂—, Cl₂CH—, CF₃CF₂—, CF₃CHF—, CHF₂CF₂—, CHF₂CHF—, CF₃CH₂—, and CH₂ClCH₂—), amino-substituted C₁₋₃ alkylene (NH₂—C₁₋₃ alkylene-, e.g., NH₂CH₂—, NH₂CH₂CH₂—, and NH₂CH₂CH₂CH₂—), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), cyano, or any combination thereof.

In some embodiments, the heterocyclylene in LIN is optionally selected from the group consisting of:
azetidinylene, oxetanylene, pyrrolidinylene, imidazolidylene, pyrazolidylene, tetrahydrofuranylene, tetrahydropyranylene, tetrahydrothienylene, tetrahydrothiopyranylene, oxazolidinylene, thiazolidinylene, piperidinylene, piperazinylene, morpholinylene, thiomorpholinylene, dioxanylene, or diazepanylene;
wherein the heterocyclylene is optionally substituted by one or more (e.g., 1-4, 1-3, 1-2, or 1) substituents selected from the group consisting of C₁₋₃ alkyl (e.g., methyl, ethyl, or propyl), C₃₋₆ cycloalkyl (e.g., cyclopropyl, cyclobutyl, cyclopentyl, or cyclohexyl), hydroxyl, amino, mercapto, halogen (e.g., fluorine, chlorine, bromine, or iodine), C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), halogenated C₁₋₃ alkyl (e.g., F₃C—, FCH₂—, F₂CH—, ClCH₂—, Cl₂CH—, CF₃CF₂—, CF₃CHF—, CHF₂CF₂—, CHF₂CHF—, CF₃CH₂—, and CH₂ClCH₂—), amino-substituted C₁₋₃ alkylene (NH₂—C₁₋₃ alkylene-, e.g., NH₂CH₂—, NH₂CH₂CH₂—, and NH₂CH₂CH₂CH₂—), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), cyano, or any combination thereof.

In some embodiments, the heteroarylene in LIN is optionally selected from the group consisting of:
furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2a]pyridylene, 1H-pyrrolo[3,2-b]pyridylene, 1H-pyrrolo[2,3-b]pyridylene, 4H-fluoro[3,2b]pyrrolylene, pyrrolo[2,1-b]thiazolylene, or imidazo[2,1-b]thiazolylene;
wherein the heteroarylene is optionally substituted by one or more (e.g., 1-4, 1-3, 1-2, or 1) substituents selected from the group consisting of C₁₋₃ alkyl (e.g., methyl, ethyl, or propyl), C₃₋₆ cycloalkyl (e.g., cyclopropyl, cyclobutyl, cyclopentyl, or cyclohexyl), hydroxyl, amino, mercapto, halogen (e.g., fluorine, chlorine, bromine, or iodine), C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), halogenated C₁₋₃ alkyl (e.g., F₃C—, FCH₂—, F₂CH—, ClCH₂—, Cl₂CH—, CF₃CF₂—, CF₃CHF—, CHF₂CF₂—, CHF₂CHF—, CF₃CH₂—, and CH₂ClCH₂—), amino-substituted C₁₋₃ alkylene (NH₂—C₁₋₃ alkylene-, e.g., NH₂CH₂—, NH₂CH₂CH₂—, and NH₂CH₂CH₂CH₂—), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), cyano, or any combination thereof.

In some embodiments, LIN represents the structure of the following formula:

#-U—CH₂—O—CH₂—, #-U—CH₂—O—(CH₂)₂—, #-U—(CH₂)₁—O—(CH₂)₃—, #-U—(CH₂)₁—O—(CH₂)₄—, #-U—(CH₂)₁—O—(CH₂)₅—, #-U—(CH₂)₁—O—(CH₂)₆—, #-U—(CH₂)₁—O—(CH₂)₇—, #-U—(CH₂)₁—O—(CH₂)₈—, #-U—(CH₂)₁—O—(CH₂)₉—, #-U—(CH₂)₁—O—(CH₂)₁₀—, #-U—(CH₂)₂—O—(CH₂)₁—, #-U—(CH₂)₂—O—(CH₂)₂—, #-U—(CH₂)₂—O—(CH₂)₃—, #-U—(CH₂)₂—O—(CH₂)₄—, #-U—(CH₂)₂—O—(CH₂)₅—, #-U—(CH₂)₂—O—(CH₂)₆—, #-U—(CH₂)₂—O—(CH₂)₇—, #-U—(CH₂)₂—O—(CH₂)₈—, #-U—(CH₂)₂—O—(CH₂)₉—, #-U—(CH₂)₂—O—(CH₂)₁₀—, #-U—(CH₂)₂—O—(CH₂)₁₁—, #-U—(CH₂)₂—O—(CH₂)₁₂—, #-U—(CH₂)₃—O—(CH₂)₁—, #-U—(CH₂)₃—O—(CH₂)₂—, #-U—(CH₂)₃—O—(CH₂)₃—, #-U—(CH₂)₃—O—(CH₂)₄—, #-U—(CH₂)₃—O—(CH₂)₅—, #-U—(CH₂)₃—O—(CH₂)₆—, #-U—(CH₂)₃—O—(CH₂)₇—, #-U—(CH₂)₄—O—(CH₂)₁—, #-U—(CH₂)₄—O—(CH₂)₂—, #-U—(CH₂)₄—O—(CH₂)₃—, #-U—(CH₂)₄—O—(CH₂)₄—, #-U—(CH₂)₄—O—(CH₂)₅—, #-U—(CH₂)₄—O—(CH₂)₆—, #-U—(CH₂)₅—O—(CH₂)₁—, #-U—(CH₂)₅—O—(CH₂)₂—, #-U—(CH₂)₅—O—(CH₂)₃—, #-U—(CH₂)₅—O—(CH₂)₄—, #-U—(CH₂)₅—O—(CH₂)₅—, #-U—(CH₂)₆—O—(CH₂)₁—, #-U—(CH₂)₆—O—(CH₂)₂—, #-U—(CH₂)₆—O—(CH₂)₃—, #-U—(CH₂)₆—O—(CH₂)₄—, #-U—(CH₂)₇—O—(CH₂)₁—, #-U—(CH₂)₇—O—(CH₂)₂—, #-U—(CH₂)₇—O—(CH₂)₃—, #-U—(CH₂)₈—O—(CH₂)₁—, #-U—(CH₂)₈—O—(CH₂)₂—, #-U—CH(CH₃)—O—(CH₂)₁—, #-U—CH(CH₃)—O—(CH₂)₂—, #-U—CH(CH₃)—O—(CH₂)₃—, #-U—CH(CH₃)—O—(CH₂)₄—, #-U—CH(CH₃)—O—(CH₂)₅—, #-U—CH(CH₃)—O—(CH₂)₆—, #-U—CH(CH₃)—O—(CH₂)₇—, #-U—CH(CH₃)—O—(CH₂)₈—, #-U—CH(CH₃)—O—(CH₂)₉—, #-U—CH(CH₃)—O—(CH₂)₁₀—, #-U—CH₂—(O(CH₂)₂)₂—, #-U—CH₂—(O(CH₂)₂)₃—, #-U—CH₂—(O(CH₂)₂)₄—, #-U—CH₂—(O(CH₂)₂)₅—, #- U—CH₂—(O(CH₂)₂)₆—, #-U—CH₂—(O(CH₂)₂)₇—, #-U—CH₂—(O(CH₂)₂)₈—, #-U—CH₂—(O(CH₂)₂)₉—, #- U—CH₂—(O(CH₂)₂)₁₀—, #-U—CH₂—(O(CH₂)₂)₁—OCH₂—, #-U—CH₂—(O(CH₂)₂)₂—OCH₂—, #-U—CH₂—(O(CH₂)₂)₃—OCH₂—, #-U—CH₂—(O(CH₂)₂)₄—OCH₂—, #-U—CH₂—(O(CH₂)₂)₅—OCH₂—, #-U—CH₂—(O(CH₂)₂)₆—OCH₂—, #-U—CH₂—(O(CH₂)₂)₇—OCH₂—, #-U—CH₂—(O(CH₂)₂)₈—OCH₂—, #-U—CH₂—(O(CH₂)₂)₉—OCH₂—, #- U—CH₂—(O(CH₂)₂)₁₀—OCH₂—, #-U—(CH₂)₂—(O(CH₂)₂)₂—, #-U—(CH₂)₂—(O(CH₂)₂)₃—, #-U—(CH₂)₂—(O(CH₂)₂)₄—, #-U—(CH₂)₂—(O(CH₂)₂)₅—, #-U—(CH₂)₂—(O(CH₂)₂)₆—, #-U—(CH₂)₂—(O(CH₂)₂)₇—, #-U—(CH₂)₂—(O(CH₂)₂)₈—, #-U—(CH₂)₂—(O(CH₂)₂)₉—, #-U—(CH₂)₂—(O(CH₂)₂)₁₀—, #-U—(CH₂)₃—(O(CH₂)₂)₂—, #-U—(CH₂)₃—(O(CH₂)₂)₃—, #-U—(CH₂)₃—(O(CH₂)₂)₄—, #-U—(CH₂)₃—(O(CH₂)₂)₅—, #-U—(CH₂)₃—(O(CH₂)₂)₆—, #-U—(CH₂)₃—(O(CH₂)₂)₇—, #-U—(CH₂)₃—(O(CH₂)₂)₈—, #-U—(CH₂)₃—(O(CH₂)₂)₉—, #-U—(CH₂)₃—(O(CH₂)₂)₁₀—, #-U—(CH₂)₄—(O(CH₂)₂)₂—, #-U—(CH₂)₄—(O(CH₂)₂)₃—, #-U—(CH₂)₄—(O(CH₂)₂)₄—, #-U—(CH₂)₄—(O(CH₂)₂)₅—, #-U—(CH₂)₄—(O(CH₂)₂)₆—, #-U—(CH₂)₄—(O(CH₂)₂)₇—, #-U—(CH₂)₄—(O(CH₂)₂)₈—, #-U—(CH₂)₄—(O(CH₂)₂)₉—, #-U—(CH₂)₄—(O(CH₂)₂)₁₀—, #-U—CH₂—(O(CH₂)₃)₂—, #-U—CH₂—(O(CH₂)₃)₃—, #-U—CH₂—(O(CH₂)₃)₄—, #-U—CH₂—(O(CH₂)₃)₅—, #-U—CH₂—(O(CH₂)₃)₆—, #-U—CH₂—(O(CH₂)₃)₇—, #-U—CH₂—(O(CH₂)₃)₈—, #-U—CH₂—(O(CH₂)₃)₉—, #- U—CH₂—(O(CH₂)₃)₁₀—, #-U—(CH₂)₂—(O(CH₂)₃)₂—, #-U—(CH₂)₂—(O(CH₂)₃)₃—, #-U—(CH₂)₂—(O(CH₂)₃)₄—, #-U—(CH₂)₂—(O(CH₂)₃)₅—, #-U—(CH₂)₂—(O(CH₂)₃)₆—, #-U—(CH₂)₂—(O(CH₂)₃)₇—, #-U—(CH₂)₂—(O(CH₂)₃)₈—, #-U—(CH₂)₂—(O(CH₂)₃)₉—, #-U—(CH₂)₂—(O(CH₂)₃)₁₀—, #-U—(CH₂)₃—(O(CH₂)₃)₂—, #-U—(CH₂)₃—(O(CH₂)₃)₃—, #-U—(CH₂)₃—(O(CH₂)₃)₄—, #-U—(CH₂)₃—(O(CH₂)₃)₅—, #-U—(CH₂)₃—(O(CH₂)₃)₆—, #-U—(CH₂)₃—(O(CH₂)₃)₇—, #-U—(CH₂)₃—(O(CH₂)₃)₈—, #-U—(CH₂)₃—(O(CH₂)₃)₉—, #-U—(CH₂)₃—(O(CH₂)₃)₁₀—, #-U—CH₂—O—(CH₂)₂—O—(CH₂)₃—, #-U—CH₂—(O(CH₂)₂)₂—(O(CH₂)₃)₂—, #-U—CH₂—(O(CH₂)₂)₃—(O(CH₂)₃)₃—, #- U—CH₂—(O(CH₂)₂)₄—(O(CH₂)₃)₄—, #-U—CH₂—(O(CH₂)₂)₅—(O(CH₂)₃)₅—, #-U—CH₂—(O(CH₂)₂)₆—(O(CH₂)₃)₆—, #-U—(CH₂)₂—O—(CH₂)₂—O—(CH₂)₃—, #-U—(CH₂)₂—(O(CH₂)₂)₂—(O(CH₂)₃)₂—, #-U—(CH₂)₂—(O(CH₂)₂)₃—(O(CH₂)₃)₃—, #-U—(CH₂)₂—(O(CH₂)₂)₄—(O(CH₂)₃)₄—, #-U—(CH₂)₂—(O(CH₂)₂)₅—(O(CH₂)₃)₅—, #-U—(CH₂)₂—(O(CH₂)₂)₆—(O(CH₂)₃)₆—, #-U—(CH₂)₃—O—(CH₂)₂—O—(CH₂)₃—, #-U—(CH₂)₃—(O(CH₂)₂)₂—(O(CH₂)₃)₂—, #-U—(CH₂)₃—(O(CH₂)₂)₃—(O(CH₂)₃)₃—, #-U—(CH₂)₃—(O(CH₂)₂)₄—(O(CH₂)₃)₄—, #-U—(CH₂)₃—(O(CH₂)₂)₅—(O(CH₂)₃)₅—, #-U—(CH₂)₃—(O(CH₂)₂)₆—(O(CH₂)₃)₆—, #-U—CH₂—O—(CH₂)₃—O—(CH₂)₂—, #-U—CH₂—(O(CH₂)₃)₂—(O(CH₂)₂)₂—, #-U—CH₂—(O(CH₂)₃)₃—(O(CH₂)₂)₃—, #-U—CH₂—(O(CH₂)₃)₄—(O(CH₂)₂)₄—, #-U—CH₂—(O(CH₂)₃)₅—(O(CH₂)₂)₅—, #-U—CH₂—(O(CH₂)₃)₆—(O(CH₂)₂)₆—, #-U—(CH₂)₂—O—(CH₂)₃—O—(CH₂)₂—, #-U—(CH₂)₂—(O(CH₂)₃)₂—(O(CH₂)₂)₂—, #-U—(CH₂)₂—(O(CH₂)₃)₃—(O(CH₂)₂)₃—, #-U—(CH₂)₂—(O(CH₂)₃)₄—(O(CH₂)₂)₄—, #-U—(CH₂)₂—(O(CH₂)₃)₅—(O(CH₂)₂)₅—, #-U—(CH₂)₂—(O(CH₂)₃)₆—(O(CH₂)₂)₆—, #-U—(CH₂)₃—O—(CH₂)₃—O—(CH₂)₂—, #-U—(CH₂)₃—(O(CH₂)₃)₂—(O(CH₂)₂)₂—, #-U—(CH₂)₃—(O(CH₂)₃)₃—(O(CH₂)₂)₃—, #-U—(CH₂)₃—(O(CH₂)₃)₄—(O(CH₂)₂)₄—, #-U—(CH₂)₃—(O(CH₂)₃)₅—(O(CH₂)₂)₅—, #-U—(CH₂)₃—(O(CH₂)₃)₆—(O(CH₂)₂)₆—, #-U—CH₂—O—(CH₂)₂O—CH₂—, #-U—(CH₂)₂—O—(CH₂)₂O—CH₂—, #-U—(CH₂)₂—(O(CH₂)₂)₂—O—(CH₂)₃—, #-U—(CH₂)₂—(O(CH₂)₂)₃—O—(CH₂)₃—, #-U—(CH₂)₂—(O(CH₂)₂)₄—O—(CH₂)₃—, #-U—(CH₂)₅—(O(CH₂)₂)₂—O—(CH₂)₅—, #-U—(CH₂)₅—(O(CH₂)₂)₂—O—(CH₂)₆—, #-U—(CH₂)₁—N(R⁷)—(CH₂)₁—, #-U—(CH₂)₁—N(R⁷)—(CH₂)₂—, #-U—(CH₂)₁—N(R⁷)—(CH₂)₃—, #-U—(CH₂)₁—N(R⁷)—(CH₂)₄—, #-U—(CH₂)₁—N(R⁷)—(CH₂)₅—, #- U—(CH₂)₁—N(R⁷)—(CH₂)₆—, #-U—(CH₂)₁—N(R⁷)—(CH₂)₇—, #-U—(CH₂)₁—N(R⁷)—(CH₂)₈—, #-U—(CH₂)₁—N(R⁷)—(CH₂)₉—, #-U—(CH₂)₁—N(R⁷)—(CH₂)₁₀—, #-U—(CH₂)₂—N(R⁷)—(CH₂)₁—, #-U—(CH₂)₂—N(R⁷)—(CH₂)₂—, #- U—(CH₂)₂—N(R⁷)—(CH₂)₃—, #-U—(CH₂)₂—N(R⁷)—(CH₂)₄—, #-U—(CH₂)₂—N(R⁷)—(CH₂)₅—, #-U—(CH₂)₂—N(R⁷)—(CH₂)₆—, #-U—(CH₂)₂—N(R⁷)—(CH₂)₇—, #-U—(CH₂)₂—N(R⁷)—(CH₂)₈—, #-U—(CH₂)₂—N(R⁷)—(CH₂)₉—, #- U—(CH₂)₂—N(R⁷)—(CH₂)₁₀—, #-U—(CH₂)₂—N(R⁷)—(CH₂)₁₁—, #-U—(CH₂)₂—N(R⁷)—(CH₂)₁₂—, #-U—(CH₂)₃—N(R⁷)—(CH₂)₁—, #-U—(CH₂)₃—N(R⁷)—(CH₂)₂—, #-U—(CH₂)₃—N(R⁷)—(CH₂)₃—, #-U—(CH₂)₄—N(R⁷)—(CH₂)₁—, #- U—(CH₂)₄—N(R⁷)—(CH₂)₂—, #-U—(CH₂)₄—N(R⁷)—(CH₂)₃—, #-U—(CH₂)₄—N(R⁷)—(CH₂)₄—, #-U—(CH₂)₅—N(R⁷)—(CH₂)₁—, #-U—(CH₂)₅—N(R⁷)—(CH₂)₂—, #-U—(CH₂)₅—N(R⁷)—(CH₂)₃—, #-U—(CH₂)₅—N(R⁷)—(CH₂)₄—, #- U—(CH₂)₅—N(R⁷)—(CH₂)₅—, #-U—(CH₂)₆—N(R⁷)—(CH₂)₁—, #-U—(CH₂)₆—N(R⁷)—(CH₂)₂—, #-U—(CH₂)₆—N(R⁷)—(CH₂)₃—, #-U—(CH₂)₇—N(R⁷)—(CH₂)₁—, #-U—(CH₂)₇—N(R⁷)—(CH₂)₂—, #-U—(CH₂)₇—N(R⁷)—(CH₂)₃—, #- U—(CH₂)₈—N(R⁷)—(CH₂)₁—, #-U—(CH₂)₈—N(R⁷)—(CH₂)₂—, #-U—(CH₂)₈—N(R⁷)—(CH₂)₃—, #-U—CH(CH₃)—N(R⁷)—(CH₂)₁—, #-U—CH(CH₃)—N(R⁷)—(CH₂)₂—, #-U—CH(CH₃)—N(R⁷)—(CH₂)₃—, #-U—CH(CH₃)—N(R⁷)—(CH₂)₄—, #-U—CH(CH₃)—N(R⁷)—(CH₂)₅—, #-U—CH(CH₃)—N(R⁷)—(CH₂)₆—, #-U—CH(CH₃)—N(R⁷)—(CH₂)₇—, #-U—CH(CH₃)—N(R⁷)—(CH₂)₈—, #-U—CH(CH₃)—N(R⁷)—(CH₂)₉—, #-U—CH(CH₃)—N(R⁷)—(CH₂)₁₀—, #-U—CH₂C(O)NHCH₂—, #-U—(CH₂)₂C(O)NH(CH₂)₂—, #-U—(CH₂)₂C(O)NH(CH₂)₃—, #-U—(CH₂)₂C(O)NH(CH₂)₄—, #-U—(CH₂)₂C(O)NH(CH₂)₅—, #-U—(CH₂)₃C(O)NH(CH₂)₃—, #-U—(CH₂)₃C(O)NH(CH₂)₄—, #-U—(CH₂)₄C(O)NH(CH₂)₄—, #-U—(CH₂)₅C(O)NH(CH₂)₅—, #-U—(CH₂)₆C(O)NH(CH₂)₇—, #-U—(CH₂)₆C(O)NH(CH₂)₆—, #-U—(CH₂)₇C(O)NH(CH₂)₇—, #-U—(CH₂)₈C(O)NH(CH₂)₈, U—(CH₂)₉C(O)NH(CH₂)₉—, #-U—(CH₂)₁₀C(O)NH(CH₂)₁₀—, #-U—(CH₂)₂C(O)NH(CH₂)₂—O—(CH₂)₂—, #-U—CH₂NHC(O)CH₂—, #-U—(CH₂)₂NHC(O)(CH₂)₂—, #-U—(CH₂)₂NHC(O)(CH₂)₃—, #-U—(CH₂)₂NHC(O)(CH₂)₄—, #-U—(CH₂)₂NHC(O)(CH₂)₅—, #-U—(CH₂)₃NHC(O)(CH₂)₃—, #-U—(CH₂)₃NHC(O)(CH₂)₄—, #-U—(CH₂)₄NHC(O)(CH₂)₄—, #-U—(CH₂)₅NHC(O)(CH₂)₅—, #-U—(CH₂)₆NHC(O)(CH₂)₇—, #-U—(CH₂)₆NHC(O)(CH₂)₆—, #-U—(CH₂)₇NHC(O)(CH₂)₇—, #-U—(CH₂)₈NHC(O)(CH₂)₈, #-U—(CH₂)₉NHC(O)(CH₂)₉—, #-U—(CH₂)₁₀NHC(O)(CH₂)₁₀—, #-U—(CH₂)₄NHC(O)(CH₂)₈—, #-U—(CH₂)₂NHC(O)(CH₂)₂—O—(CH₂)₂—, #-U—(CH₂)₄NHC(O)CH₂—, #-U—CH₂-phenylene-CH₂—, #-U—CH₂-phenylene-(CH₂)₂—, #-U—CH₂-phenylene-(CH₂)₃—, #-U—CH₂-phenylene-(CH₂)₄—, #-U—CH₂-phenylene-(CH₂)₅—, #-U—CH₂-phenylene-(CH₂)₆—, #-U—CH₂-phenylene-(CH₂)₇—, #-U—CH₂-phenylene-(CH₂)₈—, #-U—(CH₂)₂-phenylene-(CH₂)₁—, #-U—(CH₂)₂-phenylene-(CH₂)₂—, #-U—(CH₂)₂-phenylene-(CH₂)₃—, #-U—(CH₂)₂-phenylene-(CH₂)₄—, #-U—(CH₂)₂-phenylene-(CH₂)₅—, #-U—(CH₂)₂-phenylene-(CH₂)₆—, #-U—(CH₂)₂-phenylene-(CH₂)₇—, #-U—(CH₂)₂-phenylene-(CH₂)₈—, #-U—(CH₂)₃-phenylene-CH₂—, #-U—(CH₂)₃-phenylene-(CH₂)₂—, #-U—(CH₂)₃-phenylene-(CH₂)₃—, #-U—(CH₂)₃-phenylene-(CH₂)₄—, #-U—(CH₂)₃-phenylene-(CH₂)₅—, #-U—(CH₂)₃-phenylene-(CH₂)₆—, #-U—(CH₂)₃-phenylene-(CH₂)₇—, #-U—(CH₂)₃-phenylene-(CH₂)₈—, #-U—(CH₂)₄-phenylene-CH₂—, #-U—(CH₂)₄-phenylene-(CH₂)₂—, #-U—(CH₂)₄-phenylene-(CH₂)₃—, #-U—(CH₂)₄-phenylene-(CH₂)₄—, #-U—(CH₂)₄-phenylene-(CH₂)₅—, #-U—(CH₂)₄-phenylene-(CH₂)₆—, #-U—(CH₂)₄-phenylene-(CH₂)₇—, #-U—(CH₂)₄-phenylene-(CH₂)₈—, #-U—(CH₂)₅-phenylene-(CH₂)₁—, #-U—(CH₂)₅-phenylene-(CH₂)₂—, #-U—(CH₂)₅-phenylene-(CH₂)₃—, #-U—(CH₂)₅-phenylene-(CH₂)₄—, #-U—(CH₂)₅-phenylene-(CH₂)₅—, #-U—(CH₂)₅-phenylene-(CH₂)₆—, #-U—(CH₂)₅-phenylene-(CH₂)₇—, #-U—(CH₂)₅-phenylene-(CH₂)₈—, #-U—(CH₂)₆-phenylene-(CH₂)₁—, #-U—(CH₂)₆-phenylene-(CH₂)₂—, #-U—(CH₂)₆-phenylene-(CH₂)₃—, #-U—(CH₂)₆-phenylene-(CH₂)₄—, #-U—(CH₂)₆-phenylene-(CH₂)₅—, #-U—(CH₂)₆-phenylene-(CH₂)₆—, #-U—(CH₂)₆-phenylene-(CH₂)₇—, #-U—(CH₂)₆-phenylene-(CH₂)₈—, #-U—(CH₂)₇-phenylene-(CH₂)₁—, #-U—(CH₂)₇-phenylene-(CH₂)₂—, #-U—(CH₂)₇-phenylene-(CH₂)₃—, #-U—(CH₂)₇-phenylene-(CH₂)₄—, #-U—(CH₂)₇-phenylene-(CH₂)₈—, #-U—(CH₂)₈-phenylene-CH₂—, #-U—(CH₂)₈-phenylene-(CH₂)₂—, #-U—(CH₂)₈-phenylene-(CH₂)₃—, #-U—(CH₂)₈-phenylene-(CH₂)₄—, #-U—(CH₂)₈-phenylene-(CH₂)₅—, #-U—(CH₂)₈-phenylene-(CH₂)₆—, #-U—(CH₂)₈-phenylene-(CH₂)₇—, #-U—(CH₂)₈-phenylene-(CH₂)₈—, #-U—CH₂—N(R⁷)—CH₂-phenylene-CH₂—, #-U—CH₂—N(R⁷)—CH₂-phenylene-(CH₂)₂—, #-U—CH₂—N(R⁷)—CH₂-phenylene-(CH₂)₃—, #-U—CH₂—N(R⁷)—CH₂-phenylene-(CH₂)₄—, #-U—CH₂—N(R⁷)—CH₂-phenylene-(CH₂)₅—, #-U—CH₂—N(R⁷)—CH₂-phenylene-(CH₂)₆—, #-U—CH₂—N(R⁷)—CH₂-phenylene-(CH₂)₇—, #-U—CH₂—N(R⁷)—CH₂-phenylene-(CH₂)₈—, #-U—CH₂—N(R⁷)—(CH₂)₂-phenylene-CH₂—, #-U—CH₂—N(R⁷)—(CH₂)₂-phenylene-(CH₂)₂—, #-U—CH₂—N(R⁷)—(CH₂)₂-phenylene-(CH₂)₃—, #-U—CH₂—N(R⁷)—(CH₂)₂-phenylene-(CH₂)₄—, #-U—CH₂—N(R⁷)—(CH₂)₂-phenylene-(CH₂)₅—, #-U—CH₂—N(R⁷)—(CH₂)₂-phenylene-(CH₂)₆—, #-U—CH₂—N(R⁷)—(CH₂)₂-phenylene-(CH₂)₇—, #-U—CH₂—N(R⁷)—(CH₂)₂-phenylene-(CH₂)₈—, #-U—(CH₂)₂—N(R⁷)—CH₂-phenylene-CH₂—, #-U—(CH₂)₂—N(R⁷)—CH₂-phenylene-(CH₂)₂—, #-U—(CH₂)₂—N(R⁷)—CH₂-phenylene-(CH₂)₃—, #-U—(CH₂)₂—N(R⁷)—CH₂-phenylene-(CH₂)₄—, #-U—(CH₂)₂—N(R⁷)—CH₂-phenylene-(CH₂)₅—, #-U—(CH₂)₂—N(R⁷)—CH₂-phenylene-(CH₂)₆—, #-U—(CH₂)₂—N(R⁷)—CH₂-phenylene-(CH₂)₇—, #-U—(CH₂)₂—N(R⁷)—CH₂-phenylene-(CH₂)₈—, #-U—(CH₂)₃—N(R⁷)—CH₂-phenylene-CH₂—, #-U—(CH₂)₃—N(R⁷)—CH₂-phenylene-(CH₂)₂—, #-U—(CH₂)₃—N(R⁷)—CH₂-phenylene-(CH₂)₃—, #-U—(CH₂)₃—N(R⁷)—CH₂-phenylene-(CH₂)₈—, #-U—(CH₂)₄—N(R⁷)—CH₂-phenylene-CH₂—, #-U—(CH₂)₄—N(R⁷)—CH₂-phenylene-(CH₂)₂—, #-U—(CH₂)₄—N(R⁷)—CH₂-phenylene-(CH₂)₃—, #-U—(CH₂)₄—N(R⁷)—CH₂-phenylene-(CH₂)₈—, #-U—(CH₂)₅—N(R⁷)—CH₂-phenylene-(CH₂)₃—, #-U—(CH₂)₅—N(R⁷)—CH₂-phenylene-(CH₂)₈—, #-U—(CH₂)₆—N(R⁷)—CH₂-phenylene-(CH₂)₃—, #-U—(CH₂)₆—N(R⁷)—CH₂-phenylene-(CH₂)₈—, #-U—(CH₂)₇—N(R⁷)—CH₂-phenylene-(CH₂)₃—, #-U—(CH₂)₇—N(R⁷)—CH₂-phenylene-(CH₂)₈—, #-U—(CH₂)₈—N(R⁷)—CH₂-phenylene-CH₂—, #-U—(CH₂)₈—N(R⁷)—CH₂-phenylene-(CH₂)₂—, #-U—(CH₂)₈—N(R⁷)—CH₂-phenylene-(CH₂)₃—, #-U—(CH₂)₈—N(R⁷)—CH₂-phenylene-(CH₂)₄—, #-U—(CH₂)₈—N(R⁷)—CH₂-phenylene-(CH₂)₅—, #-U—(CH₂)₈—N(R⁷)—CH₂-phenylene-(CH₂)₆—, #-U—(CH₂)₈—N(R⁷)—CH₂-phenylene-(CH₂)₇—, #-U—(CH₂)₈—N(R⁷)—CH₂-phenylene-(CH₂)₈—, #-U—CH₂-piperazinylene-CH₂—, #-U—CH₂-piperazinylene-(CH₂)₂—, #-U—CH₂-piperazinylene-(CH₂)₃—, #-U—CH₂-piperazinylene-(CH₂)₄—, #-U—CH₂-piperazinylene-(CH₂)₅—, #-U—CH₂-piperazinylene-(CH₂)₆—, #-U—CH₂-piperazinylene-(CH₂)₇—, #-U—CH₂-piperazinylene-(CH₂)₈—, #-U—(CH₂)₂-piperazinylene-(CH₂)₁—, #-U—(CH₂)₂-piperazinylene-(CH₂)₂—, #-U—(CH₂)₂-piperazinylene-(CH₂)₃—, #-U—(CH₂)₂-piperazinylene-(CH₂)₄—, #-U—(CH₂)₂-piperazinylene-(CH₂)₅—, #-U—(CH₂)₂-piperazinylene-(CH₂)₆—, #-U—(CH₂)₂-piperazinylene-(CH₂)₇—, #-U—(CH₂)₂-piperazinylene-(CH₂)₈—, #-U—(CH₂)₃-piperazinylene-CH₂—, #-U—(CH₂)₃-piperazinylene-(CH₂)₂—, #-U—(CH₂)₃-piperazinylene-(CH₂)₃—, #-U—(CH₂)₃-piperazinylene-(CH₂)₄—, #-U—(CH₂)₃-piperazinylene-(CH₂)₅—, #-U—(CH₂)₃-piperazinylene-(CH₂)₆—, #-U—(CH₂)₃-piperazinylene-(CH₂)₇—, #-U—(CH₂)₃-piperazinylene-(CH₂)₈—, #-U—(CH₂)₄-piperazinylene-CH₂—, #-U—(CH₂)₄-piperazinylene-(CH₂)₂—, #-U—(CH₂)₄-piperazinylene-(CH₂)₃—, #-U—(CH₂)₄-piperazinylene-(CH₂)₄—, #-U—(CH₂)₄-piperazinylene-(CH₂)₅—, #-U—(CH₂)₄-piperazinylene-(CH₂)₆—, #-U—(CH₂)₄-piperazinylene-(CH₂)₇—, #-U—(CH₂)₄-piperazinylene-(CH₂)₈—, #-U—(CH₂)₅-piperazinylene-(CH₂)₁—, #-U—(CH₂)₅-piperazinylene-(CH₂)₂—, #-U—(CH₂)₅-piperazinylene-(CH₂)₃—, #-U—(CH₂)₅-piperazinylene-(CH₂)₄—, #-U—(CH₂)₅-piperazinylene-(CH₂)₅—, #-U—(CH₂)₅-piperazinylene-(CH₂)₆—, #-U—(CH₂)₅-piperazinylene-(CH₂)₇—, #-U—(CH₂)₅-piperazinylene-(CH₂)₈—, #-U—(CH₂)₆-piperazinylene-(CH₂)₁—, #-U—(CH₂)₆-piperazinylene-(CH₂)₂—, #-U—(CH₂)₆-piperazinylene-(CH₂)₃—, #-U—(CH₂)₆-piperazinylene-(CH₂)₄—, #-U—(CH₂)₆-piperazinylene-(CH₂)₅—, #-U—(CH₂)₆-piperazinylene-(CH₂)₆—, #-U—(CH₂)₆-piperazinylene-(CH₂)₇—, #-U—(CH₂)₆-piperazinylene-(CH₂)₈—, #-U—(CH₂)₇-piperazinylene-(CH₂)₁—, #-U—(CH₂)₇-piperazinylene-(CH₂)₂—, #-U—(CH₂)₇-piperazinylene-(CH₂)₃—, #-U—(CH₂)₇-piperazinylene-(CH₂)₄—, #-U—(CH₂)₇-piperazinylene-(CH₂)₈—, #-U—(CH₂)₈-piperazinylene-CH₂—, #-U—(CH₂)₈-piperazinylene-(CH₂)₂—, #-U—(CH₂)₈-piperazinylene-(CH₂)₃—, #-U—(CH₂)₈-piperazinylene-(CH₂)₄—, #-U—(CH₂)₈-piperazinylene-(CH₂)₅—, #-U—(CH₂)₈-piperazinylene-(CH₂)₆—, #-U—(CH₂)₈-piperazinylene-(CH₂)₇—, or #-U—(CH₂)₈-piperazinylene-(CH₂)₈—;
wherein U represents C(O) or U represents a bond, and the symbol #indicates the point of attachment to Y;
each R⁷ independently represents H or C₁₋₃ alkyl (e.g., methyl, ethyl, or propyl), and
the phenylene and the piperazinylene are each independently optionally substituted by one or more (e.g., 1-4, 1-3, 1-2, or 1) substituents selected from the group consisting of C₁₋₃ alkyl (e.g., methyl, ethyl, or propyl), C₃₋₆ cycloalkyl (e.g., cyclopropyl, cyclobutyl, cyclopentyl, or cyclohexyl), hydroxyl, amino, mercapto, halogen (e.g., fluorine, chlorine, bromine, or iodine), C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), halogenated C₁₋₃ alkyl (e.g., F₃C—, FCH₂—, F₂CH—, ClCH₂—, Cl₂CH—, CF₃CF₂—, CF₃CHF—, CHF₂CF₂—, CHF₂CHF—, CF₃CH₂—, and CH₂ClCH₂—), amino-substituted C₁₋₃ alkylene (NH₂—C₁₋₃ alkylene-, e.g., NH₂CH₂—, NH₂CH₂CH₂—, and NH₂CH₂CH₂CH₂—), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), cyano, or any combination thereof.

In some embodiments, the compound of Formula (I) of the present disclosure is also of Formula (II):

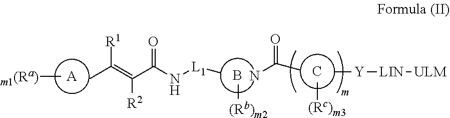

or salts (including pharmaceutically acceptable salts), solvates, isotopically enriched analogs, polymorphs, prodrugs, stereoisomers (including enantiomers), or mixture of stereoisomers thereof,
wherein ULM is a E3 ligase ligand moiety, LIN is a linker moiety, the remaining moiety of the compound of Formula (I) is a nicotinamide phosphoribosyltransferase (NAMPT) ligand, and ULM is covalently bonded to NAMPT ligand through LIN;
wherein ring B is a 5- to 11-membered heterocyclylene group containing 1 nitrogen atom and optionally containing from 1 to 2 heteroatoms independently selected from the group consisting of nitrogen, oxygen, and sulfur, and (R^{b})ₘ₂ indicates that ring B is optionally substituted by m2 R^{b} groups, wherein each R^{b} is independently C₁₋₃ alkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, or cyano, and m2 represents an integer of 0, 1, 2, 3, 4, or 5;
ring A is the following groups:

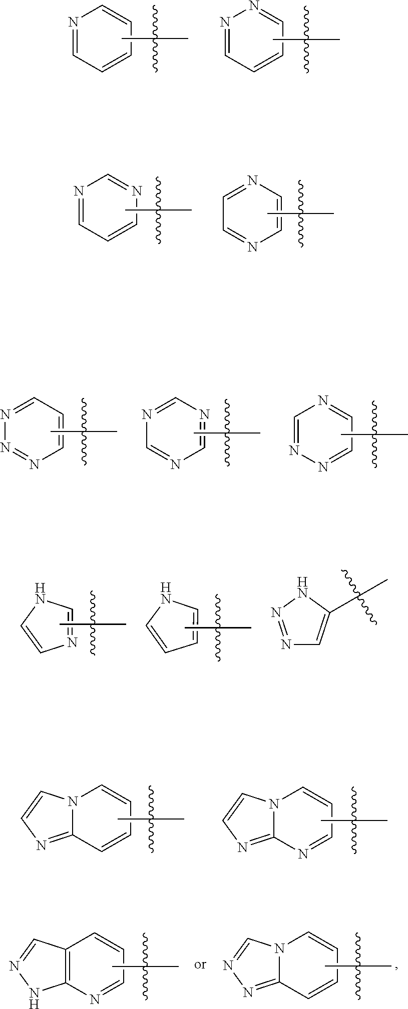

(R^{a})ₘ₁ indicates that the ring A is optionally substituted by m1 R^{a} groups, wherein each R^{a} is independently hydroxyl, amino, halogen, or cyano, and m1 represents an integer of 0, 1, 2, 3, 4, or 5;
R¹ and R² are the same or different and each independently represent H, cyano, or methyl;
L₁ represents substituted or unsubstituted linear C₃₋₆ alkylene group, or substituted or unsubstituted linear C₃₋₆ alkenylene group, wherein the optional substituents of the linear C₃₋₆ alkylene group and the linear C₃₋₆ alkenylene group are selected from the group consisting of C₁₋₃ alkyl, halogen, C₁₋₃ alkoxy, halogenated C₁₋₃ alkyl, C₁₋₃ alkyl-NHC(O)—, C₁₋₃ alkyl-C(O)NH—, cyano, or any combination thereof;
ring C is a 5- to 10-membered heteroarylene group or a 6-membered arylene group, and (R^{c})ₘ₃ indicates that ring C is optionally substituted by m3 R^{c} groups, wherein each R^{c} is independently C₁₋₃ alkyl, C₃₋₅ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, or cyano, and m3 represents an integer of 0, 1, 2, 3, 4, or 5;
m represents an integer of 0 or 1; and
Y represents —N(R³)—, where R³ is H or C₁₋₃ alkyl; or
Y represents —O—; or
Y is n connected rings D represented by the following formula:

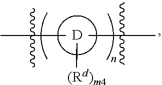

each ring D is independently a 5- to 11-membered heterocyclylene group, and (R^{d})ₘ₄ indicates that each ring D is optionally independently substituted by m4 R^{d} groups, wherein each R^{d} is independently C₁₋₃ alkyl, C₃₋₆ cycloalkyl, hydroxyl, amino, mercapto, halogen, oxo, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, or cyano, and m4 represents an integer of 0, 1, 2, 3, 4, or 5;
n represents an integer of 0, 1, 2, or 3, wherein when n represents an integer of 2 or 3, each ring D can be the same or different, and
where m and n are not both 0 at the same time;
with the proviso that the following compounds are excluded:
the nicotinamide phosphoribosyltransferase (NAMPT) ligand represents the following structure:

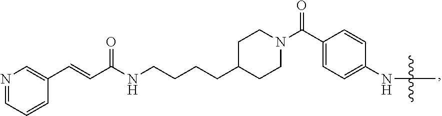 [SMILES: *Nc1ccc(C(=O)N2CCC(CCCCNC(=O)/C=C/c3cccnc3)CC2)cc1]

and
ULM represents a structure of Formula (IV):

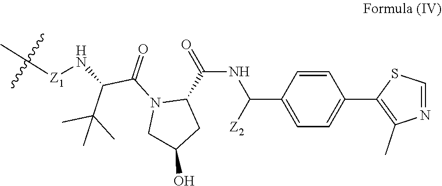

wherein Z₁ represents C(O) or Z₁ represents a bond, and Z₂ represents H or CH₃.

In some embodiments, L₁ of the compound of Formula (II) represents substituted or unsubstituted linear C₃₋₆ alkylene group. Exemplary examples of linear C₃₋₆ alkylene group include, but are not limited to, propylene, butylene, pentylene, and hexylene. The linear C₃₋₆ alkylene group is optionally further substituted with one or more substituents selected from the group consisting of C₁₋₃ alkyl (e.g., methyl, ethyl, or propyl), halogen (e.g., fluorine, chlorine, bromine, or iodine), C₁₋₃ alkoxy (e.g., methoxy, ethoxy, or propoxy), halo-substituted C₁₋₃ alkyl (e.g., —CF₃, —CH₂F, —CHF₂, —CH₂Cl, —CHCl₂, —CF₂CF₃, —CHFCF₃, —CF₂CHF₂, —CHFCHF₂, —CH₂CF₃, and —CH₂CH₂Cl), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), cyano, or any combination thereof. The number of substituents is in principle not limited in any way, or is automatically limited by the size of the building unit.

In some embodiments, L₁ of the compound of Formula (II) represents substituted or unsubstituted linear C₃₋₆ alkenylene group. Exemplary examples of linear C₃₋₆ alkenylene group include, but are not limited to,

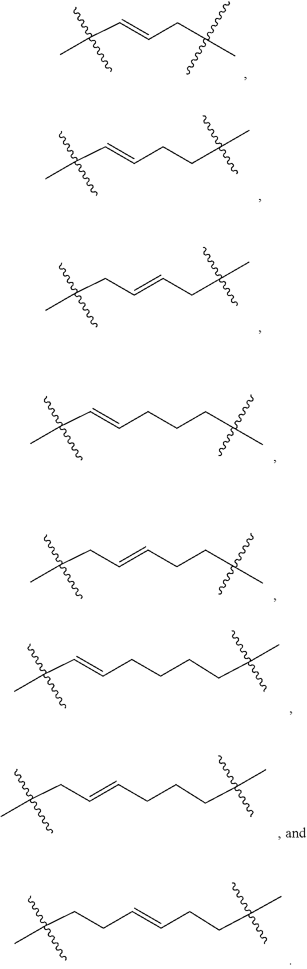

The linear C₃₋₆ alkenylene is optionally further substituted with one or more substituents selected from the group consisting of C₁₋₃ alkyl (e.g., methyl, ethyl, or propyl), halogen (e.g., fluorine, chlorine, bromine, or iodine), C₁₋₃ alkoxy (e.g., methoxy, ethoxy, or propoxy), halo-substituted C₁₋₃ alkyl (e.g., —CF₃, —CH₂F, —CHF₂, —CH₂Cl, —CHCl₂, —CF₂CF₃, —CHFCF₃, —CF₂CHF₂, —CHFCHF₂, —CH₂CF₃, and —CH₂CH₂Cl), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), cyano, or any combination thereof. The number of substituents is in principle not limited in any way, or is automatically limited by the size of the building unit.

In some embodiments, ring B of the compound of Formula (II) is a 5- to 11-membered heterocyclylene containing from 1 to 3 nitrogen atoms.

In some embodiments, ring B of the compound of Formula (II) is piperidinylene, piperazinylene, morpholinylene, azetidinylene, pyrrolidinylene, imidazolidylene, pyrazolidylene, oxazolidinylene, thiazolidinylene, thiomorpholinylene, diazepanylene, or C₇₋₁₁ spiroheterocyclylene containing 1 nitrogen atom and optionally containing from 1 to 2 heteroatoms independently selected from the group consisting of nitrogen, oxygen, and sulfur.

In some embodiments, when ring B of the compound of Formula (II) is C₇₋₁₁ spiroheterocyclylene containing 1 nitrogen atom and optionally containing from 1 to 2 heteroatoms independently selected from the group consisting of nitrogen, oxygen, and sulfur, the C₇₋₁₁ spiroheterocyclylene is the following groups:

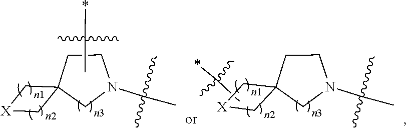

wherein the symbol * indicates the point of attachment to L₁, X represents O, N(R^{e}), S, or CH₂, or X represents a bond, where R^{e} represents H or methyl, and n1, n2, and n3 are each independently an integer of 1 or 2.

In some embodiments, when ring B of the compound of Formula (II) is C₇₋₁₁ spiroheterocyclylene containing 1 nitrogen atom and optionally containing from 1 to 2 heteroatoms independently selected from the group consisting of nitrogen, oxygen, and sulfur, the C₇₋₁₁ spiroheterocyclylene is the following groups:

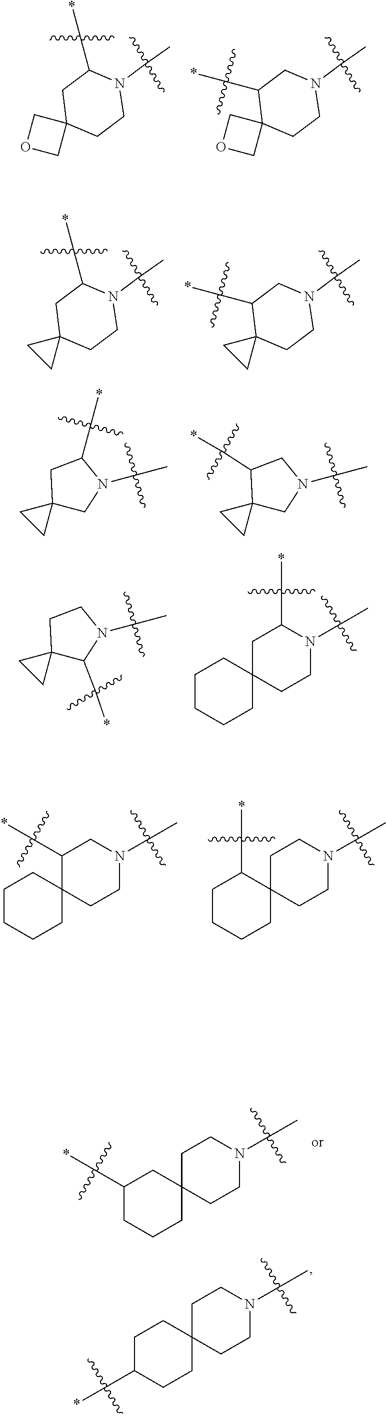

wherein the symbol * indicates the point of attachment to L₁.

In some embodiments, ring B of the compound of Formula (II) is optionally further substituted by m2 R^{b} groups, wherein m2 represents an integer of 0, 1, 2, 3, 4, or 5, and each R^{b} is independently C₁₋₃ alkyl (e.g., methyl, ethyl, or propyl), hydroxyl, amino, mercapto, halogen (e.g., fluorine, chlorine, bromine, or iodine), C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), halogenated C₁₋₃ alkyl (e.g., —CF₃, —CH₂F, —CHF₂, —CH₂Cl, —CHCl₂, —CF₂CF₃, —CHFCF₃, —CF₂CHF₂, —CHFCHF₂, —CH₂CF₃, and —CH₂CH₂Cl), amino-substituted C₁₋₃ alkylene (NH₂—C₁₋₃ alkylene-, e.g., NH₂CH₂—, NH₂CH₂CH₂—, and NH₂CH₂CH₂CH₂—), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), or cyano.

In some embodiments, exemplary examples of

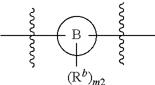

of the compound of Formula (II) include, but are not limited to, the following groups:

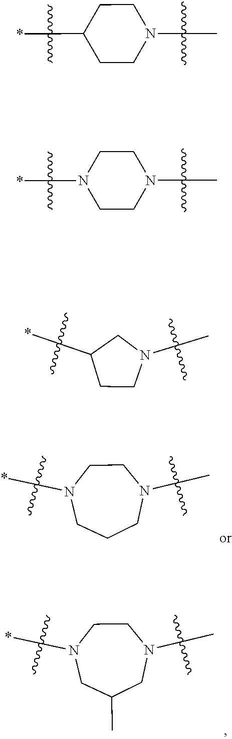

wherein the symbol * indicates the point of attachment to L₁.

In some embodiments, ring C of the compound of Formula (II) is a 5- to 10-membered heteroarylene group or a 6-membered arylene group. Exemplary examples of ring C include, but are not limited to, the following groups: phenylene, pyridylene, pyrimidinylene, pyrazinylene, pyridazinylene, 1,2,4-triazinylene, 1,3,5-triazinylene, triazolylene, furanylene, oxazolylene, isoxazolylene, oxadiazolylene, thienylene, thiazolylene, isothiazolylene, thiadiazolylene, pyrrolylene, imidazolylene, pyrazolylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2a]pyridylene, 1H-pyrrolo[3,2-b]pyridylene, 1H-pyrrolo[2,3-b]pyridylene, 4H-fluoro[3,2b]pyrrolylene, pyrrolo[2,1-b]thiazolylene, or imidazo[2,1-b]thiazolylene.

In some embodiments, ring C of the compound of Formula (II) is optionally further substituted by m3 R^{c} groups, wherein m3 represents an integer of 0, 1, 2, 3, 4, or 5, and each R^{c} is independently C₁₋₃ alkyl (e.g., methyl, ethyl, or propyl), C₃₋₅ cycloalkyl (e.g., cyclopropyl, cyclobutyl, or cyclopentyl), hydroxyl, amino, mercapto, halogen (e.g., fluorine, chlorine, bromine, or iodine), C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), halogenated C₁₋₃ alkyl (e.g., —CF₃, —CH₂F, —CHF₂, —CH₂Cl, —CHCl₂, —CF₂CF₃, —CHFCF₃, —CF₂CHF₂, —CHFCHF₂, —CH₂CF₃, and —CH₂CH₂Cl), amino-substituted C₁₋₃ alkylene (NH₂—C₁₋₃ alkylene-, e.g., NH₂CH₂—, NH₂CH₂CH₂—, and NH₂CH₂CH₂CH₂—), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), or cyano.

In some embodiments, exemplary examples of

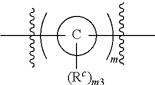

of the compound of Formula (II) include, but are not limited to, the following groups:

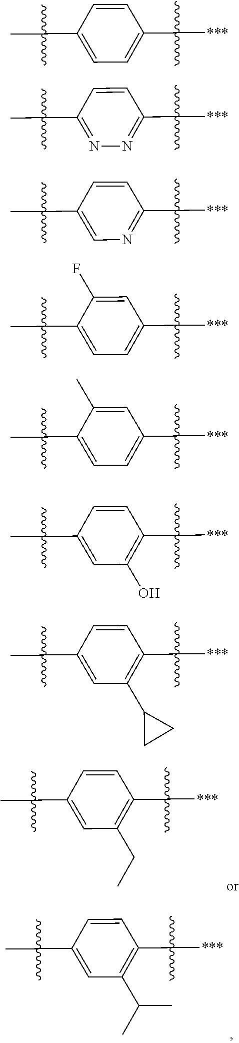

wherein the symbol *** indicates the point of attachment to Y, or the symbol *** indicates the point of attachment to carbonyl.

In some embodiments, Y of the compound of Formula (II) represents —N(R³)—, where R³ is H or C₁₋₃ alkyl.

In some embodiments, Y of the compound of Formula (II) represents —O—.

In some embodiments, Y of the compound of Formula (II) is n connected rings D represented by the following formula:

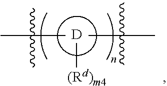

wherein n represents an integer of 0, 1, 2, or 3, wherein when n represents an integer of 2 or 3, each ring D can be the same or different, and each ring D is independently a 5- to 11-membered heterocyclylene group, and (R^{d})ₘ₄ indicates that each ring D is optionally independently substituted by m4 R^{d} groups, wherein m4 represents an integer of 0, 1, 2, 3, 4, or 5, and each R^{d} is independently C₁₋₃ alkyl, C₃₋₆ cycloalkyl, hydroxyl, amino, mercapto, halogen, oxo, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, or cyano.

In some embodiments, exemplary examples of ring D of the compound of Formula (II) include, but are not limited to, the following groups: piperidinylene, piperazinylene, morpholinylene, azetidinylene, oxetanylene, pyrrolidinylene, imidazolidylene, pyrazolidylene, tetrahydrofuranylene, tetrahydropyranylene, tetrahydrothienylene, tetrahydrothiopyranylene, oxazolidinylene, thiazolidinylene, thiomorpholinylene, dioxanylene, diazepanylene, or C₇₋₁₁ spiroheterocyclylene.

In some embodiments, when ring D of the compound of Formula (II) represents C₇₋₁₁ spiroheterocyclylene, the C₇₋₁₁ spiroheterocyclylene can be the following groups:

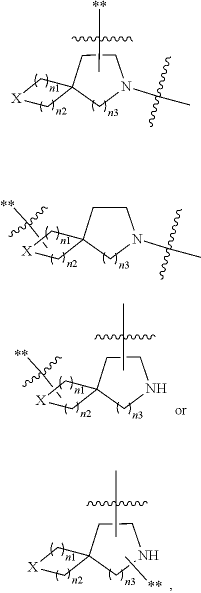

wherein the symbol ** indicates the point of attachment to ring C, X represents O, N(R^{e}), S, or CH₂, or X represents a bond, where R^{e} represents H or methyl, and n1, n2, and n3 are each independently an integer of 1 or 2.

In some embodiments, when ring D of the compound of Formula (II) represents C₇₋₁₁ spiroheterocyclylene, the C₇₋₁₁ spiroheterocyclylene can be the following groups:

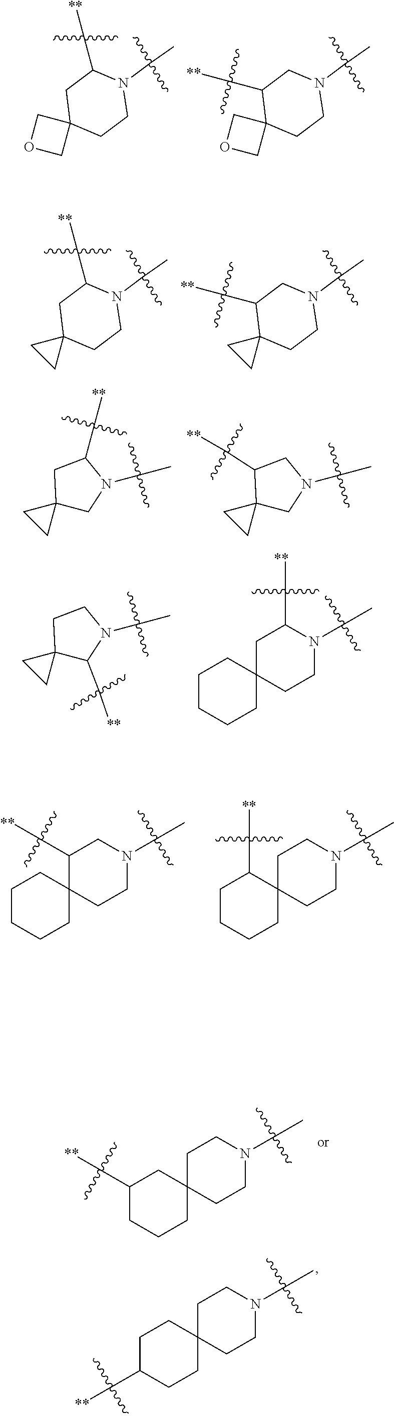

wherein the symbol ** indicates the point of attachment to ring C.

In some embodiments, ring D of the compound of Formula (II) is optionally further substituted by m4 R^{d} groups, wherein m4 represents an integer of 0, 1, 2, 3, 4, or 5; and each R^{d} is independently C₁₋₃ alkyl (e.g., methyl, ethyl, or propyl), C₃₋₆ cycloalkyl (e.g., cyclopropyl, cyclobutyl, cyclopentyl, or cyclohexyl), hydroxyl, amino, mercapto, halogen (e.g., fluorine, chlorine, bromine, or iodine), oxo, C₁₋₃ alkoxy (e.g., methoxy, ethoxy, or propoxy), C₁₋₃ alkylamino (e.g., C₁₋₃ alkyl-NH—, e.g., CH₃NH—, CH₃CH₂NH—, or CH₃CH₂CH₂NH—), halogenated C₁₋₃ alkyl (e.g., —CF₃, —CH₂F, —CHF₂, —CH₂Cl, —CHCl₂, —CF₂CF₃, —CHFCF₃, —CF₂CHF₂, —CHFCHF₂, —CH₂CF₃, and —CH₂CH₂Cl), amino-substituted C₁₋₃ alkylene (NH₂—C₁₋₃ alkylene-, e.g., NH₂CH₂—, NH₂CH₂CH₂—, and NH₂CH₂CH₂CH₂—), C₁₋₃ alkyl-NHC(O)— (e.g., CH₃—NHC(O)—, CH₃CH₂—NHC(O)—, and CH₃CH₂CH₂—NHC(O)—), C₁₋₃ alkyl-C(O)NH— (e.g., CH₃—C(O)NH—, CH₃CH₂—C(O)NH—, and CH₃CH₂CH₂—C(O)NH—), or cyano.

In some embodiments, exemplary examples of

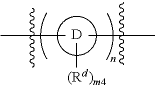

of the compound of Formula (II) include, but are not limited to, the following groups:

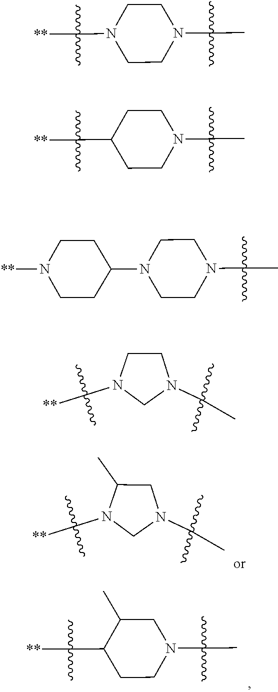

wherein the symbol ** indicates the point of attachment to ring C. Alternatively, in some embodiments, the symbol ** can also indicate the point of attachment to LIN.

In some embodiments, exemplary examples of

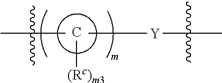

of the compound of Formula (II) include, but are not limited to, the following groups:

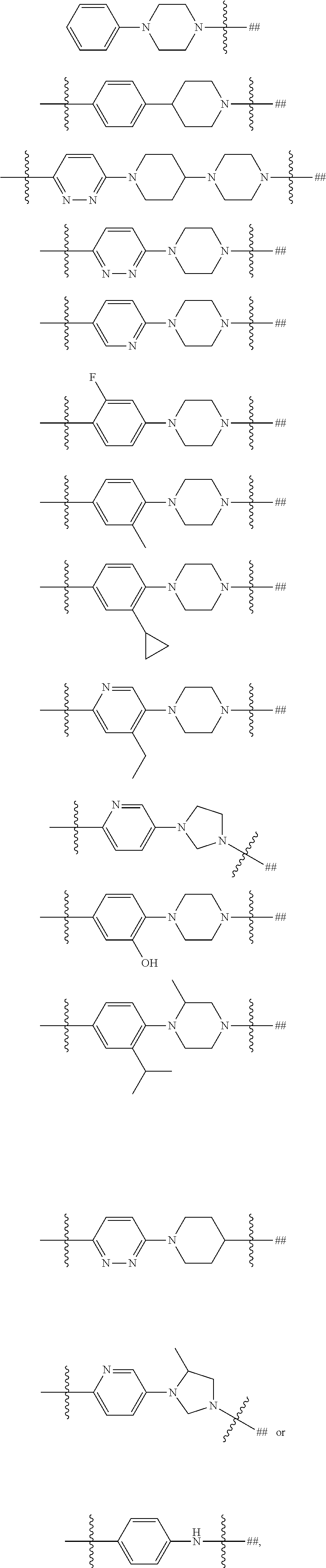

wherein the symbol ##indicates the point of attachment to LIN.

ULM and LIN of the compound of Formula (II) of the present disclosure are as defined in the embodiments or sub-embodiments of the compounds of Formula (I) herein.

In some embodiments, the compound of Formula (I) of the present disclosure is also of Formula (V):

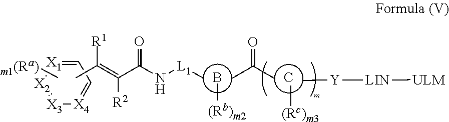

or salts (including pharmaceutically acceptable salts), solvates, isotopically enriched analogs, polymorphs, prodrugs, stereoisomers (including enantiomers), or mixture of stereoisomers thereof,
wherein any one, two, or three of X₁, X₂, X₃, and X₄ represent N, and the remaining represent CH; and
ULM, LIN, Y, ring C, (R^{c})ₘ₃, m, ring B, (R^{b})ₘ₂, L₁, R¹, R², (R^{a})ₘ₁ are as defined in any one of the embodiments or sub-embodiments of the compounds of Formula (I) herein.

In some embodiments, the compound of Formula (I) of the present disclosure is also of Formula (VI):

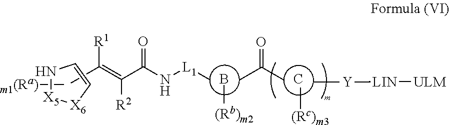

or salts (including pharmaceutically acceptable salts), solvates, isotopically enriched analogs, polymorphs, prodrugs, stereoisomers (including enantiomers), or mixture of stereoisomers thereof,
wherein X₅ and X₆ are the same or different and each independently represent CH or N; and
ULM, LIN, Y, ring C, (R^{c})ₘ₃, m, ring B, (R^{b})ₘ₂, L₁, R¹, R², (R^{a})ₘ₁ are as defined in any one of the embodiments or sub-embodiments of the compounds of Formula (I) herein.

In some embodiments, the compound of Formula (I) of the present disclosure is also of Formula (VII):

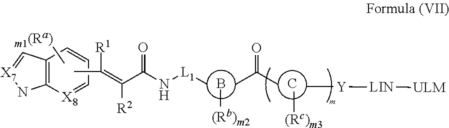

or salts (including pharmaceutically acceptable salts), solvates, isotopically enriched analogs, polymorphs, prodrugs, stereoisomers (including enantiomers), or mixture of stereoisomers thereof,
wherein any one of X₇ and X₈ represents CH, and the other represents N; or X₇ and X₈ represent CH; and
ULM, LIN, Y, ring C, (R^{c})ₘ₃, m, ring B, (R^{b})ₘ₂, L₁, R¹, R², (R^{a})ₘ₁ are as defined in any one of the embodiments or sub-embodiments of the compounds of Formula (I) herein.

In some embodiments, the compound of Formula (I) of the present disclosure is also of Formula (VIII):

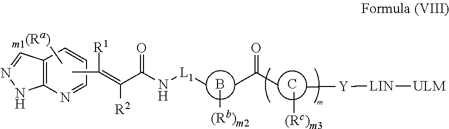

or salts (including pharmaceutically acceptable salts), solvates, isotopically enriched analogs, polymorphs, prodrugs, stereoisomers (including enantiomers), or mixture of stereoisomers thereof,
wherein ULM, LIN, Y, ring C, (R^{c})ₘ₃, m, ring B, (R^{b})ₘ₂, L₁, R¹, R², (R^{a})ₘ₁ are as defined in any one of the embodiments or sub-embodiments of the compounds of Formula (I) herein.

It will be understood by those skilled in the art that the present invention encompasses compounds resulting from any combination of the various embodiments. Embodiments obtained by combining technical features or preferred technical features of one embodiment with technical features or preferred technical features of another embodiment are also encompassed within the scope of the present invention as defined by the appended claims.

In some embodiments, the compounds of the present invention and their salts (especially pharmaceutically acceptable salts, such as hydrochloride, etc.), prodrugs, solvates, isotopically enriched analogs, polymorphs, stereoisomers (including enantiomers and diastereomers), or mixture of stereoisomers thereof in Table 1 are provided.

TABLE 1
The compounds of the present disclosure

| Compound | | Chinese translation of | The compounds' |
|---|---|---|---|
| No. | Structure of the compounds | the compounds' name | name |
| SIAI S630 009 | 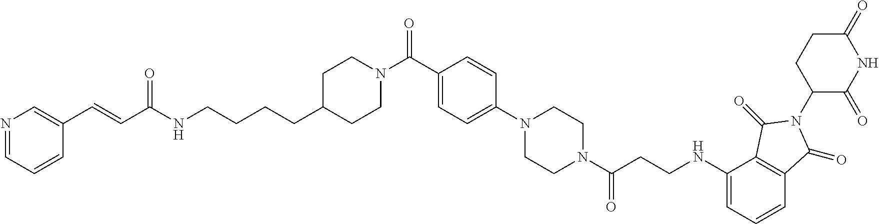 [SMILES: O=C(/C=C/c1cccnc1)NCCCCC1CCN(C(=O)c2ccc(N3CCN(C(=O)CCNc4cccc5c4C(=O)N(C4CCC(=O)NC4=O)C5=O)CC3)cc2)CC1] | (E)-N-(4-(1-(4-(4-(3- ((2-(2,6-dioxopiperidin- 3-yl)-1,3- dioxoisoindolin-4- yl)amino)propanoyl) piperazin-1- yl)benzoyl)piperidin-4- yl)butyl)-3-(pyridin-3- yl)acrylamide | (E)-N-(4-(1-(4-(4-(3- ((2-(2,6- dioxopiperidin-3-yl)- 1,3-dioxoisoindolin-4- yl)amino)propanoyl) piperazin-1- yl)benzoyl)piperidin-4- yl)butyl)-3-(pyridin-3- yl)acrylamide |
| SIAI S630 010 | 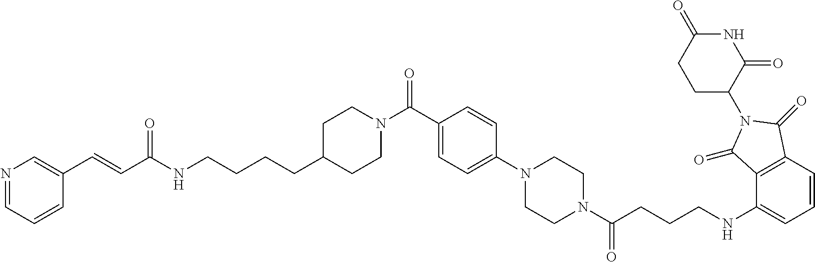 [SMILES: O=C(/C=C/c1cccnc1)NCCCCC1CCN(C(=O)c2ccc(N3CCN(C(=O)CCCNc4cccc5c4C(=O)N(C4CCC(=O)NC4=O)C5=O)CC3)cc2)CC1] | (E)-N-(4-(1-(4-(4-(4- ((2-(2,6-dioxopiperidin- 3-yl)-1,3- dioxoisoindolin-4- yl)amino)butanoyl) piperazin-1- yl)benzoyl)piperidin-4- yl)butyl)-3-(pyridin-3- yl)acrylamide | (E)-N-(4-(1-(4-(4-(4- ((2-(2,6- dioxopiperidin-3-yl)- 1,3-dioxoisoindolin-4- yl)amino)butanoyl) piperazin-1- yl)benzoyl)piperidin-4- yl)butyl)-3-(pyridin-3- yl)acrylamide |
| SIAI S630 011 | 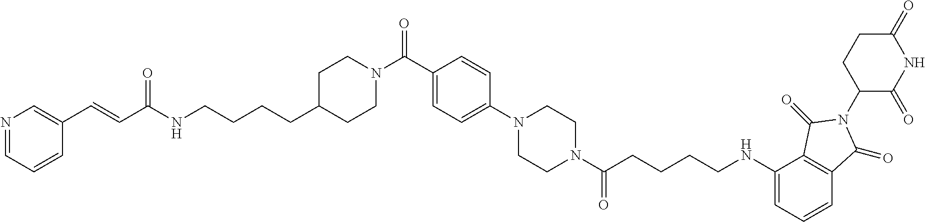 [SMILES: O=C(/C=C/c1cccnc1)NCCCCC1CCN(C(=O)c2ccc(N3CCN(C(=O)CCCCNc4cccc5c4C(=O)N(C4CCC(=O)NC4=O)C5=O)CC3)cc2)CC1] | (E)-N-(4-(1-(4-(4-(5- ((2-(2,6-dioxopiperidin- 3-yl)-1,3- dioxoisoindolin-4- yl)amino)pentanoyl) piperazin-1- yl)benzoyl)piperidin-4- yl)butyl)-3-(pyridin-3- yl)acrylamide | (E)-N-(4-(1-(4-(4-(5- ((2-(2,6- dioxopiperidin-3-yl)- 1,3-dioxoisoindolin-4- yl)amino)pentanoyl) piperazin-1- yl)benzoyl)piperidin-4- yl)butyl)-3-(pyridin-3- yl)acrylamide |
| SIAI S632 014 | 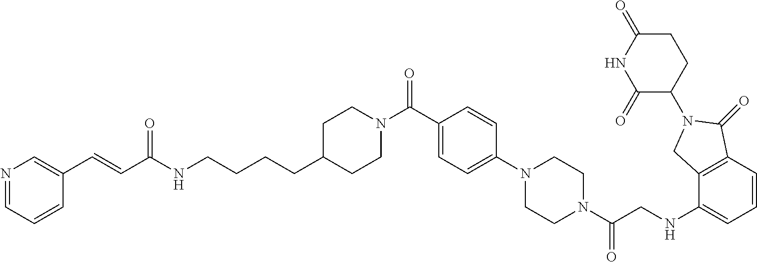 [SMILES: O=C(/C=C/c1cccnc1)NCCCCC1CCN(C(=O)c2ccc(N3CCN(C(=O)CNc4cccc5c4CN(C4CCC(=O)NC4=O)C5=O)CC3)cc2)CC1] | (E)-N-(4-(1-(4-(4-((2- (2,6-dioxopiperidin-3- yl)-1-oxoisoindolin-4- yl)glycyl)piperazin-1- yl)benzoyl)piperidin-4- yl)butyl)-3-(pyridin-3- yl)acrylamide | (E)-N-(4-(1-(4-(4-((2- (2,6-dioxopiperidin-3- yl)-1-oxoisoindolin-4- yl)glycyl)piperazin-1- yl)benzoyl)piperidin-4- yl)butyl)-3-(pyridin-3- yl)acrylamide |
| SIAI S632 015 | 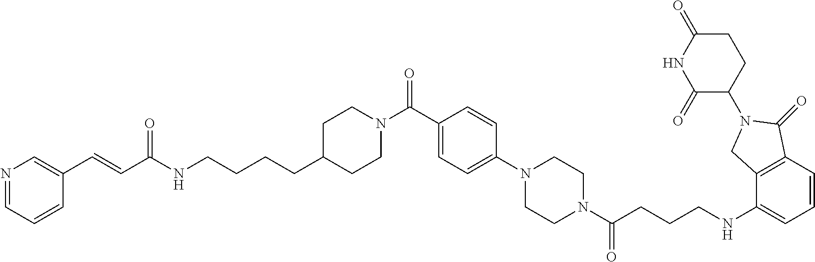 [SMILES: O=C(/C=C/c1cccnc1)NCCCCC1CCN(C(=O)c2ccc(N3CCN(C(=O)CCCNc4cccc5c4CN(C4CCC(=O)NC4=O)C5=O)CC3)cc2)CC1] | (E)-N-(4-(1-(4-(4-(4- ((2-(2,6-dioxopiperidin- 3-yl)-1-oxoisoindolin-4- yl)amino)butanoyl) piperazin-1- yl)benzoyl)piperidin-4- yl)butyl)-3-(pyridin-3- yl)acrylamide | (E)-N-(4-(1-(4-(4-(4- ((2-(2,6- dioxopiperidin-3-yl)-1- oxoisoindolin-4- yl)amino)butanoyl) piperazin-1- yl)benzoyl)piperidin-4- yl)butyl)-3-(pyridin-3- yl)acrylamide |
| SIAI S632 016 | 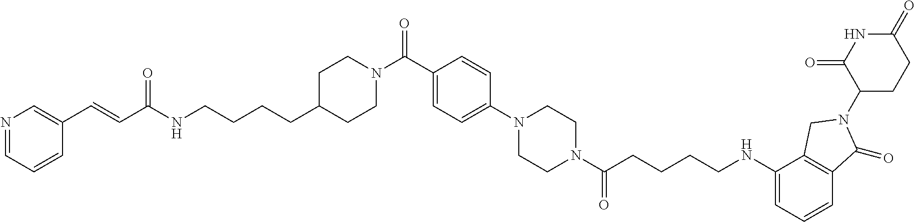 [SMILES: O=C(/C=C/c1cccnc1)NCCCCC1CCN(C(=O)c2ccc(N3CCN(C(=O)CCCCNc4cccc5c4CN(C4CCC(=O)NC4=O)C5=O)CC3)cc2)CC1] | (E)-N-(4-(1-(4-(4-(5- ((2-(2,6-dioxopiperidin- 3-yl)-1-oxoisoindolin-4- yl)amino)pentanoyl) piperazin-1- yl)benzoyl)piperidin-4- yl)butyl)-3-(pyridin-3- yl)acrylamide | (E)-N-(4-(1-(4-(4-(5- ((2-(2,6- dioxopiperidin-3-yl)-1 - oxoisoindolin-4- yl)amino)pentanoyl) piperazin-1- yl)benzoyl)piperidin-4- yl)butyl)-3-(pyridin-3- yl)acrylamide |
| SIAI S632 017 | 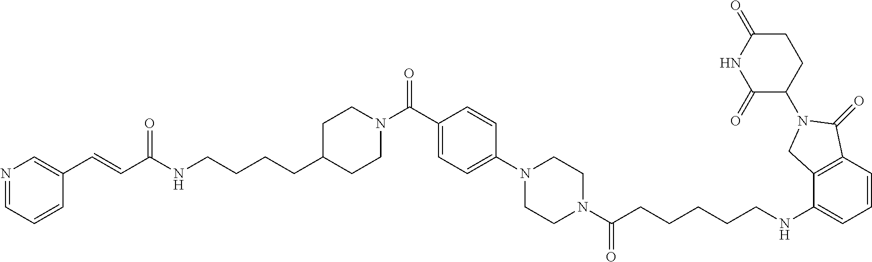 [SMILES: O=C(/C=C/c1cccnc1)NCCCCC1CCN(C(=O)c2ccc(N3CCN(C(=O)CCCCCNc4cccc5c4CN(C4CCC(=O)NC4=O)C5=O)CC3)cc2)CC1] | (E)-N-(4-(1-(4-(4-(6- ((2-(2,6-dioxopiperidin- 3-yl)-1-oxoisoindolin-4- yl)amino)hexanoyl) piperazin-1- yl)benzoyl)piperidin-4- yl)butyl)-3-(pyridin-3- yl)acrylamide | (E)-N-(4-(1-(4-(4-(6- ((2-(2,6- dioxopiperidin-3-yl)-1- oxoisoindolin-4- yl)amino)hexanoyl) piperazin-1- yl)benzoyl)piperidin-4- yl)butyl)-3-(pyridin-3- yl)acrylamide |
| SIAI S632 018 | 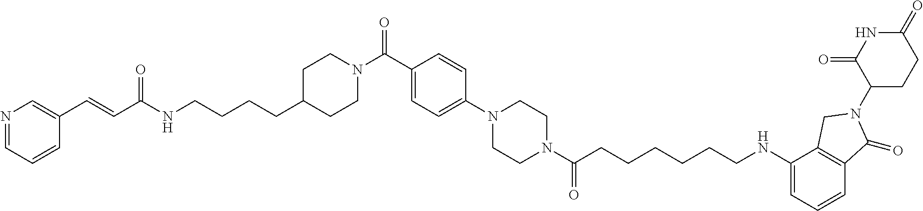 [SMILES: O=C(/C=C/c1cccnc1)NCCCCC1CCN(C(=O)c2ccc(N3CCN(C(=O)CCCCCCNc4cccc5c4CN(C4CCC(=O)NC4=O)C5=O)CC3)cc2)CC1] | (E)-N-(4-(1-(4-(4-(7-+ ((2-(2,6-dioxopiperidin- 3-yl)-1-oxoisoindolin- 4-yl)amino)heptanoyl) piperazin-1- yl)benzoyl)piperidin-4- yl)butyl)-3-(pyridin-3- yl)acrylamide | (E)-N-(4-(1-(4-(4-(7- ((2-(2,6- dioxopiperidin-3-yl)-1- oxoisoindolin-4- yl)amino)heptanoyl) piperazin-1- yl)benzoyl)piperidin-4- yl)butyl)-3-(pyridin-3- yl)acrylamide |
| SIAI S630 012 | 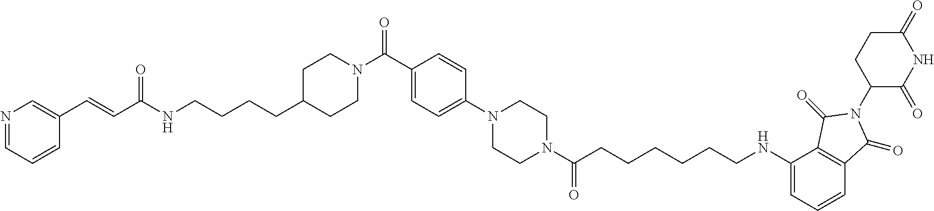 [SMILES: O=C(/C=C/c1cccnc1)NCCCCC1CCN(C(=O)c2ccc(N3CCN(C(=O)CCCCCCNc4cccc5c4C(=O)N(C4CCC(=O)NC4=O)C5=O)CC3)cc2)CC1] | (E)-N-(4-(1-(4-(4-(7- ((2-(2,6-dioxopiperidin- 3-yl)-1,3- dioxoisoindolin-4- yl)amino)heptanoyl) piperazin-1- yl)benzoyl)piperidin-4- yl)butyl)-3-(pyridin-3- yl)acrylamide | (E)-N-(4-(1-(4-(4-(7- ((2-(2,6- dioxopiperidin-3-yl)- 1,3-dioxoisoindolin-4- yl)amino)heptanoyl) piperazin-1- yl)benzoyl)piperidin-4- yl)butyl)-3-(pyridin-3- yl)acrylamide |
| SIAI S630 013 | 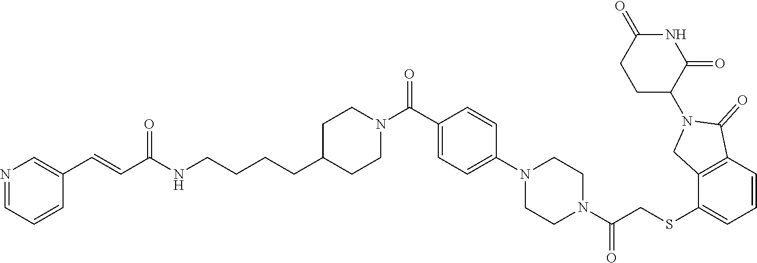 [SMILES: O=C(/C=C/c1cccnc1)NCCCCC1CCN(C(=O)c2ccc(N3CCN(C(=O)CSc4cccc5c4CN(C4CCC(=O)NC4=O)C5=O)CC3)cc2)CC1] | (E)-N-(4-(1-(4-(4-(2- ((2-(2,6-dioxopiperidin- 3-yl)-1-oxoisoindolin-4- yl)thio)acetyl)piperazin- 1-yl)benzoyl)piperidin- 4-yl)butyl)-3-(pyridin-3- yl)acrylamide | (E)-N-(4-(1-(4-(4-(2- ((2-(2,6- dioxopiperidin-3-yl)-1- oxoisoindolin-4- yl)thio)acetyl) piperazin-1- yl)benzoyl)piperidin-4- yl)butyl)-3-(pyridin-3- yl)acrylamide |
| SIAI S630 014 | 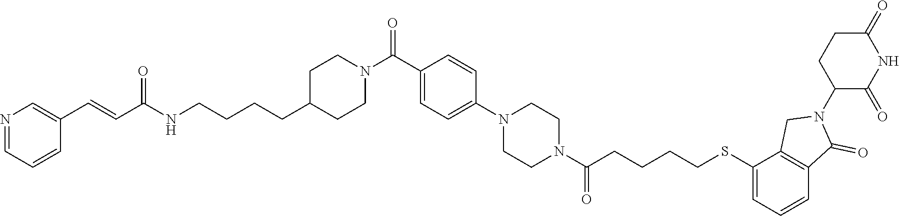 [SMILES: O=C(/C=C/c1cccnc1)NCCCCC1CCN(C(=O)c2ccc(N3CCN(C(=O)CCCCSc4cccc5c4CN(C4CCC(=O)NC4=O)C5=O)CC3)cc2)CC1] | (E)-N-(4-(1-(4-(4-(5- ((2-(2,6-dioxopiperidin- 3-yl)-1-oxoisoindolin-4- yl)thio)pentanoyl) piperazin-1- yl)benzoyl)piperidin-4- yl)butyl)-3-(pyridin-3- yl)acrylamide | (E)-N-(4-(1-(4-(4-(5- ((2-(2,6- dioxopiperidin-3-yl)-1- oxoisoindolin-4- yl)thio)pentanoyl) piperazin-1- yl)benzoyl)piperidin-4- yl)butyl)-3-(pyridin-3- yl)acrylamide |
| SIAI S630 037 | 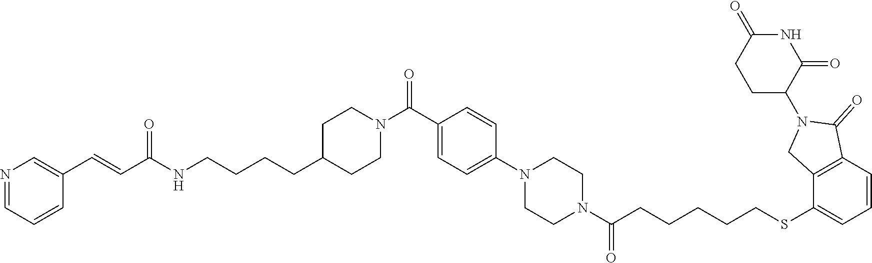 [SMILES: O=C(/C=C/c1cccnc1)NCCCCC1CCN(C(=O)c2ccc(N3CCN(C(=O)CCCCCSc4cccc5c4CN(C4CCC(=O)NC4=O)C5=O)CC3)cc2)CC1] | (E)-N-(4-(1-(4-(4-(6- ((2-(2,6-dioxopiperidin- 3-yl)-1-oxoisoindolin-4- yl)thio)hexanoyl) piperazin-1- yl)benzoyl)piperidin-4- yl)butyl)-3-(pyridin-3- yl)acrylamide | (E)-N-(4-(1-(4-(4-(6- ((2-(2,6- dioxopiperidin-3-yl)-1- oxoisoindolin-4- yl)thio)hexanoyl) piperazin-1- yl)benzoyl)piperidin-4- yl)butyl)-3-(pyridin-3- yl)acrylamide |
| SIAI S630 038 | 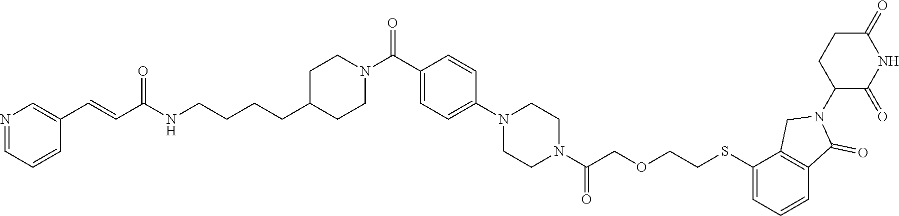 [SMILES: O=C(/C=C/c1cccnc1)NCCCCC1CCN(C(=O)c2ccc(N3CCN(C(=O)COCCSc4cccc5c4CN(C4CCC(=O)NC4=O)C5=O)CC3)cc2)CC1] | (E)-N-(4-(1-(4-(4-(2-(2- ((2-(2,6-dioxopiperidin- 3-yl)-1-oxoisoindolin-4- yl)thio)ethoxy)acetyl) piperazin-1- yl)benzoyl)piperidin-4- yl)butyl)-3-(pyridin-3- yl)acrylamide | (E)-N-(4-(1-(4-(4-(2- (2-((2-(2,6- dioxopiperidin-3-yl)-1- oxoisoindolin-4- yl)thio)ethoxy)acetyl) piperazin-1- yl)benzoyl)piperidin-4- yl)butyl)-3-(pyridin-3- yl)acrylamide |
| SIAI S630 039 | 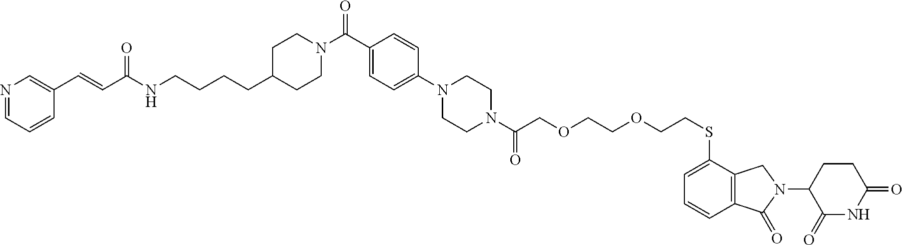 [SMILES: O=C(/C=C/c1cccnc1)NCCCCC1CCN(C(=O)c2ccc(N3CCN(C(=O)COCCOCCSc4cccc5c4CN(C4CCC(=O)NC4=O)C5=O)CC3)cc2)CC1] | (E)-N-(4-(1-(4-(4-(2-(2- (2-((2-(2,6- dioxopiperidin-3-yl)-1- oxoisoindolin-4- yl)thio)ethoxy)ethoxy) acetyl)piperazin-1- yl)benzoyl)piperidin-4- yl)butyl)-3-(pyridin-3- yl)acrylamide | (E)-N-(4-(1-(4-(4-(2- (2-(2-((2-(2,6- dioxopiperidin-3-yl)-1- oxoisoindolin-4- yl)thio)ethoxy)ethoxy) acetyl)piperazin-1- yl)benzoyl)piperidin-4- yl)butyl)-3-(pyridin-3- yl)acrylamide |
| SIAI S630 130 | 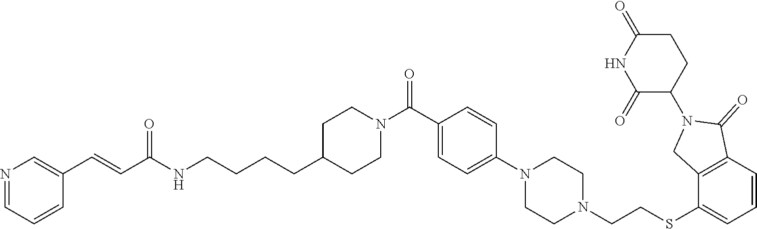 [SMILES: O=C(/C=C/c1cccnc1)NCCCCC1CCN(C(=O)c2ccc(N3CCN(CCSc4cccc5c4CN(C4CCC(=O)NC4=O)C5=O)CC3)cc2)CC1] | (E)-N-(4-(1-(4-(4-(2- ((2-(2,6-dioxopiperidin- 3-yl)-1-oxoisoindolin-4- yl)thio)ethyl)piperazin- 1-yl)benzoyl)piperidin- 4-yl)butyl)-3-(pyridin-3- yl)acrylamide | (E)-N-(4-(1-(4-(4-(2- ((2-(2,6- dioxopiperidin-3-yl)-1- oxoisoindolin-4- yl)thio)ethyl)piperazin- 1-yl)benzoyl)piperidin- 4-yl)butyl)-3-(pyridin- 3-yl)acrylamide |
| SIAI S630 131 | 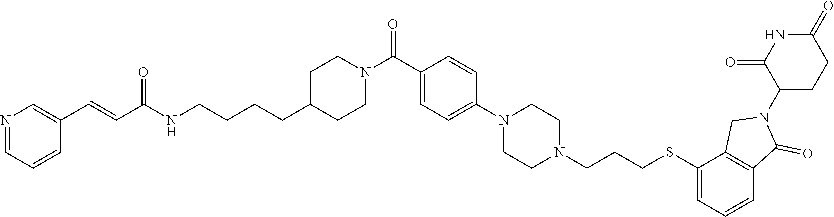 [SMILES: O=C(/C=C/c1cccnc1)NCCCCC1CCN(C(=O)c2ccc(N3CCN(CCCSc4cccc5c4CN(C4CCC(=O)NC4=O)C5=O)CC3)cc2)CC1] | (E)-N-(4-(1-(4-(4-(3- ((2-(2,6-dioxopiperidin- 3-yl)-1-oxoisoindolin-4- yl)thio)propyl)piperazin- 1-yl)benzoyl)piperidin- 4-yl)butyl)-3-(pyridin-3- yl)acrylamide | (E)-N-(4-(1-(4-(4-(3- ((2-(2,6- dioxopiperidin-3-yl)-1- oxoisoindolin-4- yl)thio)propyl) piperazin-1- yl)benzoyl)piperidin-4- yl)butyl)-3-(pyridin-3- yl)acrylamide |
| SIAI S630 123 | 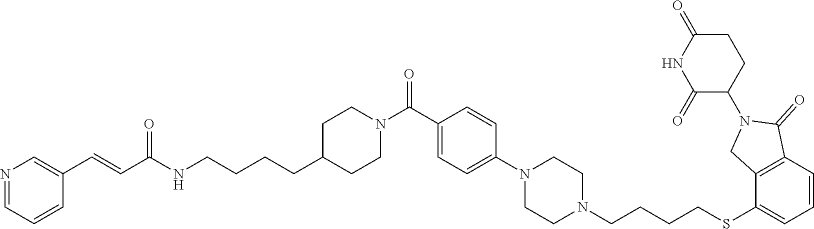 [SMILES: O=C(/C=C/c1cccnc1)NCCCCC1CCN(C(=O)c2ccc(N3CCN(CCCCSc4cccc5c4CN(C4CCC(=O)NC4=O)C5=O)CC3)cc2)CC1] | (E)-N-(4-(1-(4-(4-(4- ((2-(2,6-dioxopiperidin- 3-yl)-1-oxoisoindolin-4- yl)thio)butyl)piperazin- 1-yl)benzoyl)piperidin- 4-yl)butyl)-3-(pyridin-3- yl)acrylamide | (E)-N-(4-(1-(4-(4-(4- ((2-(2,6- dioxopiperidin-3-yl)-1- oxoisoindolin-4- yl)thio)butyl)piperazin- 1-yl)benzoyl)piperidin- 4-yl)butyl)-3-(pyridin- 3-yl)acrylamide |
| SIAI S630 132 | 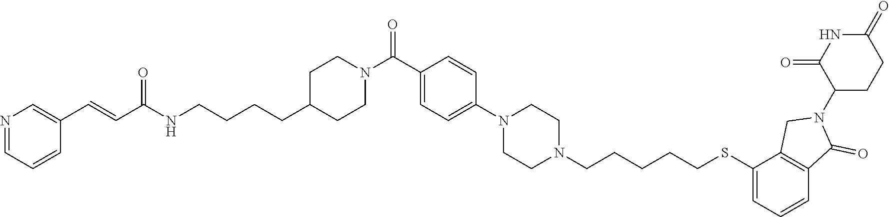 [SMILES: O=C(/C=C/c1cccnc1)NCCCCC1CCN(C(=O)c2ccc(N3CCN(CCCCCSc4cccc5c4CN(C4CCC(=O)NC4=O)C5=O)CC3)cc2)CC1] | (E)-N-(4-(1-(4-(4-(5- ((2-(2,6-dioxopiperidin- 3-yl)-1-oxoisoindolin-4- yl)thio)pentyl)piperazin- 1-yl)benzoyl)piperidin- 4-yl)butyl)-3-(pyridin-3- yl)acrylamide | (E)-N-(4-(1-(4-(4-(5- ((2-(2,6- dioxopiperidin-3-yl)-1- oxoisoindolin-4- yl)thio)pentyl) piperazin-1- yl)benzoyl)piperidin-4- yl)butyl)-3-(pyridin-3- yl)acrylamide |
| SIAI S630 133 | 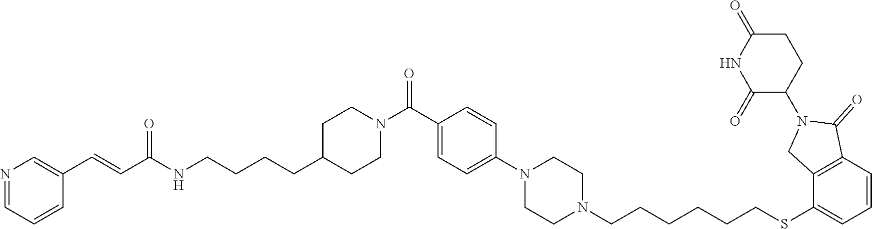 [SMILES: O=C(/C=C/c1cccnc1)NCCCCC1CCN(C(=O)c2ccc(N3CCN(CCCCCCSc4cccc5c4CN(C4CCC(=O)NC4=O)C5=O)CC3)cc2)CC1] | (E)-N-(4-(1-(4-(4-(6- ((2-(2,6-dioxopiperidin- 3-yl)-1-oxoisoindolin-4- yl)thio)hexyl)piperazin- 1-yl)benzoyl)piperidin- 4-yl)butyl)-3-(pyridin-3- yl)acrylamide | (E)-N-(4-(1-(4-(4-(6- ((2-(2,6- dioxopiperidin-3-yl)- 1-oxoisoindolin-4- yl)thio)hexyl) piperazin-1- yl)benzoyl)piperidin-4- yl)butyl)-3-(pyridin-3- yl)acrylamide |
| SIAI S630 134 | 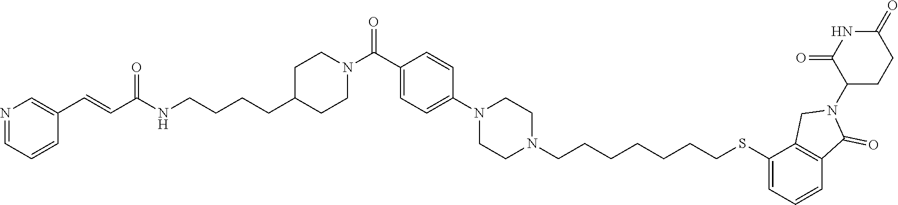 [SMILES: O=C(/C=C/c1cccnc1)NCCCCC1CCN(C(=O)c2ccc(N3CCN(CCCCCCCSc4cccc5c4CN(C4CCC(=O)NC4=O)C5=O)CC3)cc2)CC1] | (E)-N-(4-(1-(4-(4-(7- ((2-(2,6-dioxopiperidin- 3-yl)-1-oxoisoindolin-4- yl)thio)heptyl)piperazin- 1-yl)benzoyl)piperidin- 4-yl)butyl)-3-(pyridin-3- yl)acrylamide | (E)-N-(4-(1-(4-(4-(7- ((2-(2,6- dioxopiperidin-3-yl)- 1-oxoisoindolin-4- yl)thio)heptyl) piperazin-1- yl)benzoyl)piperidin-4- yl)butyl)-3-(pyridin-3- yl)acrylamide |
| SIAI S630 135 | 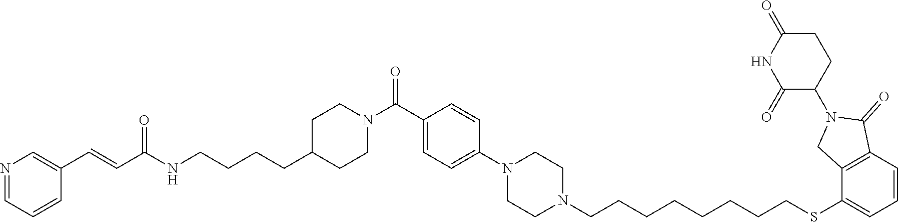 [SMILES: O=C(/C=C/c1cccnc1)NCCCCC1CCN(C(=O)c2ccc(N3CCN(CCCCCCCCSc4cccc5c4CN(C4CCC(=O)NC4=O)C5=O)CC3)cc2)CC1] | (E)-N-(4-(1-(4-(4-(8- ((2-(2,6-dioxopiperidin- 3-yl)-1-oxoisoindolin-4- yl)thio)octyl)piperazin- 1-yl)benzoyl)piperidin- 4-yl)butyl)-3-(pyridin-3- yl)acrylamide | (E)-N-(4-(1-(4-(4-(8- ((2-(2,6- dioxopiperidin-3-yl)-1- oxoisoindolin-4- yl)thio)octyl)piperazin- 1-yl)benzoyl)piperidin- 4-yl)butyl)-3-(pyridin- 3-yl)acrylamide |
| SIAI S630 136 | 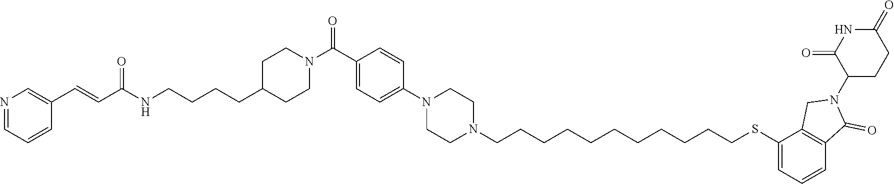 [SMILES: O=C(/C=C/c1cccnc1)NCCCCC1CCN(C(=O)c2ccc(N3CCN(CCCCCCCCCCCSc4cccc5c4CN(C4CCC(=O)NC4=O)C5=O)CC3)cc2)CC1] | (E)-N-(4-(1-(4-(4-(11- ((2-(2,6-dioxopiperidin- 3-yl)-1-oxoisoindolin-4- yl)thio)undecyl) piperazin-1- yl)benzoyl)piperidin-4- yl)butyl)-3-(pyridin-3- yl)acrylamide | (E)-N-(4-(1-(4-(4-(11- ((2-(2,6- dioxopiperidin-3-yl)-1- oxoisoindolin-4- yl)thio)undecyl) piperazin-1- yl)benzoyl)piperidin-4- yl)butyl)-3-(pyridin-3- yl)acrylamide |
| SIAI S630 129 | 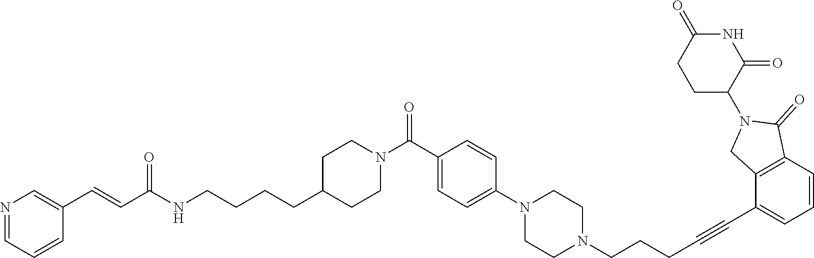 [SMILES: O=C(/C=C/c1cccnc1)NCCCCC1CCN(C(=O)c2ccc(N3CCN(CCCC#Cc4cccc5c4CN(C4CCC(=O)NC4=O)C5=O)CC3)cc2)CC1] | (E)-N-(4-(1-(4-(4-(5-(2- (2,6-dioxopiperidin-3- yl)-1-oxoisoindolin-4- yl)pent-4-yn-1- yl)piperazin-1- yl)benzoyl)piperidin-4- yl)butyl)-3-(pyridin-3- yl)acrylamide | (E)-N-(4-(1-(4-(4-(5- (2-(2,6-dioxopiperidin- 3-yl)-1-oxoisoindolin- 4-yl)pent-4-yn-1- yl)piperazin-1- yl)benzoyl)piperidin-4- yl)butyl)-3-(pyridin-3- yl)acrylamide |
| SIAI S630 119 | 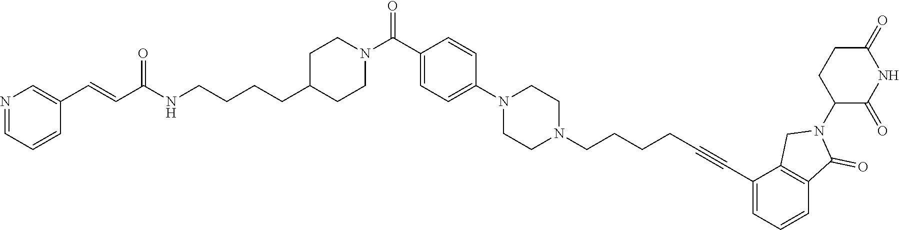 [SMILES: O=C(/C=C/c1cccnc1)NCCCCC1CCN(C(=O)c2ccc(N3CCN(CCCCC#Cc4cccc5c4CN(C4CCC(=O)NC4=O)C5=O)CC3)cc2)CC1] | (E)-N-(4-(1-(4-(4-(6-(2- (2,6-dioxopiperidin-3- yl)-1-oxoisoindolin-4- yl)hex-5-yn-1- yl)piperazin-1- yl)benzoyl)piperidin-4- yl)butyl)-3-(pyridin-3- yl)acrylamide | (E)-N-(4-(1-(4-(4-(6- (2-(2,6-dioxopiperidin- 3-yl)-1-oxoisoindolin- 4-yl)hex-5-yn-1- yl)piperazin-1- yl)benzoyl)piperidin-4- yl)butyl)-3-(pyridin-3- yl)acrylamide |
| SIAI S630 120 | 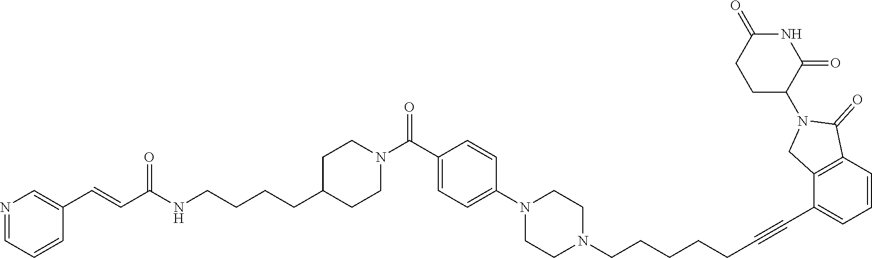 [SMILES: O=C(/C=C/c1cccnc1)NCCCCC1CCN(C(=O)c2ccc(N3CCN(CCCCCC#Cc4cccc5c4CN(C4CCC(=O)NC4=O)C5=O)CC3)cc2)CC1] | (E)-N-(4-(1-(4-(4-(7-(2- (2,6-dioxopiperidin-3- yl)-1-oxoisoindolin-4- yl)hept-6-yn-1- yl)piperazin-1- yl)benzoyl)piperidin-4- yl)butyl)-3-(pyridin-3- yl)acrylamide | (E)-N-(4-(1-(4-(4-(7- (2-(2,6-dioxopiperidin- 3-yl)-1-oxoisoindolin- 4-yl)hept-6-yn-1- yl)piperazin-1- yl)benzoyl)piperidin-4- yl)butyl)-3-(pyridin-3- yl)acrylamide |
| SIAI S630 121 | 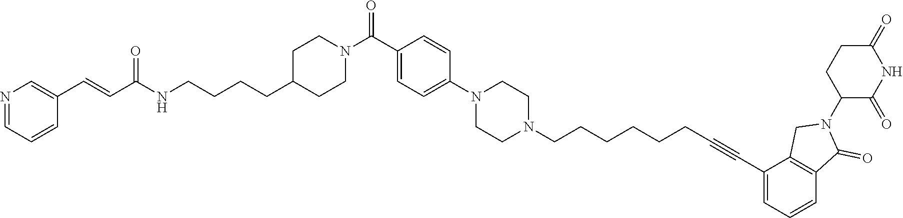 [SMILES: O=C(/C=C/c1cccnc1)NCCCCC1CCN(C(=O)c2ccc(N3CCN(CCCCCCC#Cc4cccc5c4CN(C4CCC(=O)NC4=O)C5=O)CC3)cc2)CC1] | (E)-N-(4-(1-(4-(4-(8-(2- (2,6-dioxopiperidin-3- yl)-1-oxoisoindolin-4- yl)oct-7-yn-1- yl)piperazin-1- yl)benzoyl)piperidin-4- yl)butyl)-3-(pyridin-3- yl)acrylamide | (E)-N-(4-(1-(4-(4-(8- (2-(2,6-dioxopiperidin- 3-yl)-1-oxoisoindolin- 4-yl)oct-7-yn-1- yl)piperazin-1- yl)benzoyl)piperidin-4- yl)butyl)-3-(pyridin-3- yl)acrylamide |
| SIAI S630 122 | 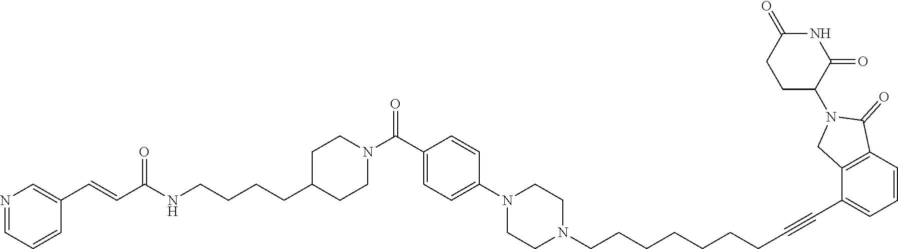 [SMILES: O=C(/C=C/c1cccnc1)NCCCCC1CCN(C(=O)c2ccc(N3CCN(CCCCCCCC#Cc4cccc5c4CN(C4CCC(=O)NC4=O)C5=O)CC3)cc2)CC1] | (E)-N-(4-(1-(4-(4-(9-(2- (2,6-dioxopiperidin-3- yl)-1-oxoisoindolin-4- yl)non-8-yn-1- yl)piperazin-1- yl)benzoyl)piperidin-4- yl)butyl)-3-(pyridin-3- yl)acrylamide | (E)-N-(4-(1-(4-(4-(9- (2-(2,6-dioxopiperidin- 3-yl)-1-oxoisoindolin- 4-yl)non-8-yn-1- yl)piperazin-1- yl)benzoyl)piperidin-4- yl)butyl)-3-(pyridin-3- yl)acrylamide |
| SIAI S631 001 | 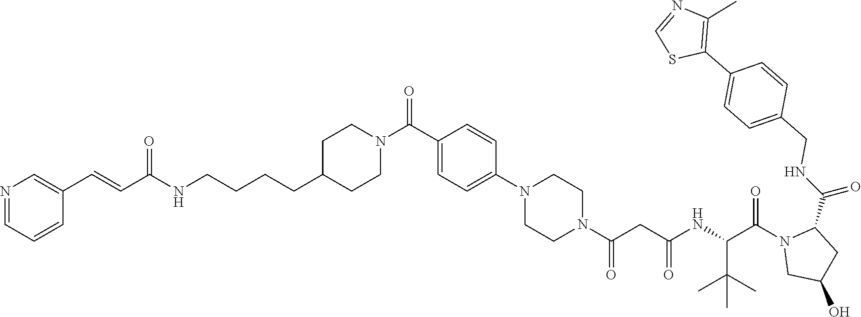 [SMILES: Cc1ncsc1-c1ccc(CNC(=O)[C@@H]2C[C@@H](O)CN2C(=O)[C@@H](NC(=O)CC(=O)N2CCN(c3ccc(C(=O)N4CCC(CCCCNC(=O)/C=C/c5cccnc5)CC4)cc3)CC2)C(C)(C)C)cc1] | (2S,4R)-1-((S)-3,3- dimethyl-2-(3-oxo-3-(4- (4-(4-(4-((E)-3-(pyridin- 3-yl)acrylamido)butyl) piperidine-1- carbonyl)phenyl) piperazin-1- yl)propanamido) butanoyl)-4- hydroxy-N-(4-(4- methylthiazol-5- yl)benzyl)pyrrolidine-2- carboxamide | (2S,4R)-1-((S)-3,3- dimethyl-2-(3-oxo-3- (4-(4-(4-(4-((E)-3- (pyridin-3- yl)acrylamido)butyl) piperidine-1- carbonyl)phenyl) piperazin-1- yl)propanamido) butanoyl)- 4-hydroxy-N-(4-(4- methylthiazol-5- yl)benzyl)pyrrolidine- 2-carboxamide |
| SIAI S631 002 | 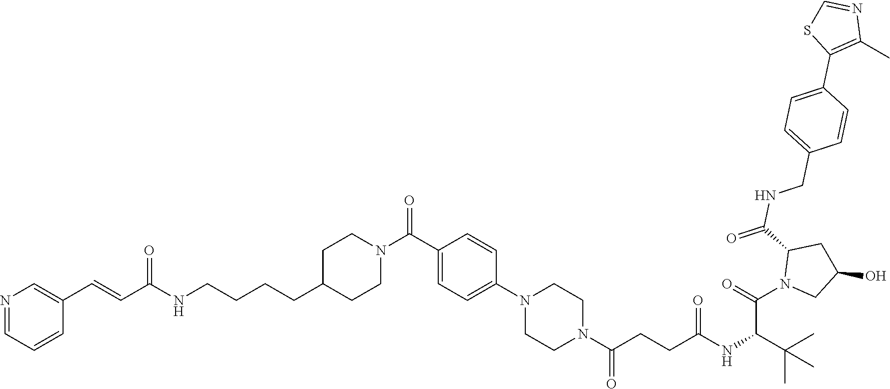 [SMILES: Cc1ncsc1-c1ccc(CNC(=O)[C@@H]2C[C@@H](O)CN2C(=O)[C@@H](NC(=O)CCC(=O)N2CCN(c3ccc(C(=O)N4CCC(CCCCNC(=O)/C=C/c5cccnc5)CC4)cc3)CC2)C(C)(C)C)cc1] | (2S,4R)-1-((S)-3,3- dimethyl-2-(4-oxo-4-(4- (4-(4-(4-((E)-3- (pyridin-3- yl)acrylamido)butyl) piperidine-1- carbonyl)phenyl) piperazin-1- yl)butanamido) butanoyl )-4-hydroxy-N-(4-(4- methylthiazol-5- yl)benzyl)pyrrolidine-2- carboxamide | (2S,4R)-1-((S)-3,3- dimethyl-2-(4-oxo-4- (4-(4-(4-(4-((E)-3- (pyridin-3- yl)acrylamido)butyl) piperidine-1- carbonyl)phenyl) piperazin-1- yl)butanamido) butanoyl)- 4-hydroxy-N-(4-(4- methylthiazol-5- yl)benzyl)pyrrolidine- 2-carboxamide |
| SIAI S631 003 | 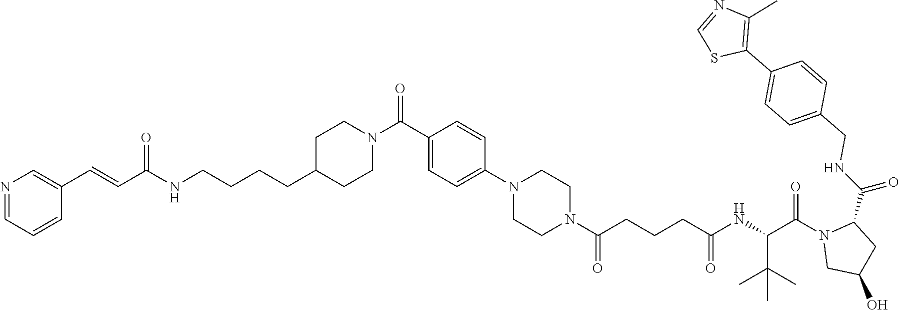 [SMILES: Cc1ncsc1-c1ccc(CNC(=O)[C@@H]2C[C@@H](O)CN2C(=O)[C@@H](NC(=O)CCCC(=O)N2CCN(c3ccc(C(=O)N4CCC(CCCCNC(=O)/C=C/c5cccnc5)CC4)cc3)CC2)C(C)(C)C)cc1] | (2S,4R)-1-((S)-3,3- dimethyl-2-(5-oxo-5-(4- (4-(4-(4-((E)-3- (pyridin-3- yl)acrylamido)butyl) piperidine-1- carbonyl)phenyl) piperazin-1- yl)pentanamido) butanoyl)-4- hydroxy-N-(4-(4- methylthiazol-5- yl)benzyl)pyrrolidine-2- carboxamide | (2S,4R)-1-((S)-3,3- dimethyl-2-(5-oxo-5- (4-(4-(4-(4-((E)-3- (pyridin-3- yl)acrylamido)butyl) piperidine-1- carbonyl)phenyl) piperazin-1- yl)pentanamido) butanoyl)-4- hydroxy-N-(4-(4- methylthiazol-5- yl)benzyl)pyrrolidine- 2-carboxamide |
| SIAI S630 007 | 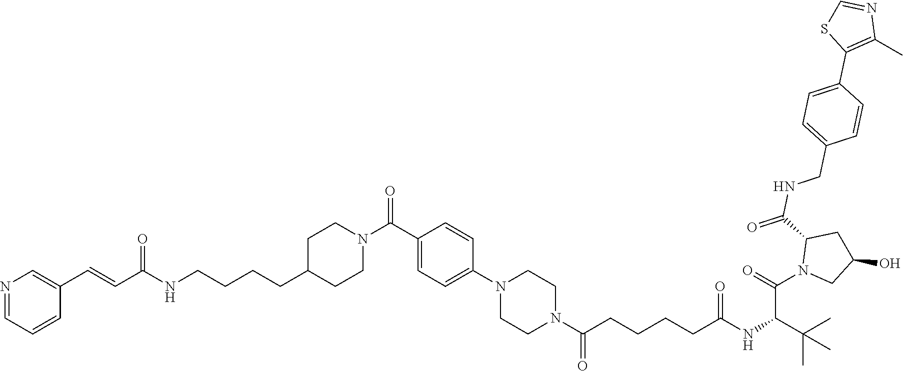 [SMILES: Cc1ncsc1-c1ccc(CNC(=O)[C@@H]2C[C@@H](O)CN2C(=O)[C@@H](NC(=O)CCCCC(=O)N2CCN(c3ccc(C(=O)N4CCC(CCCCNC(=O)/C=C/c5cccnc5)CC4)cc3)CC2)C(C)(C)C)cc1] | (2S,4R)-1-((S)-3,3- dimethyl-2-(6-oxo-6-(4- (4-(4-(4-((E)-3- (pyridin-3- yl)acrylamido)butyl) piperidine-1- carbonyl)phenyl) piperazin-1-yl) hexanamido)butanoyl)- 4-hydroxy-N-(4-(4- methylthiazol-5- yl)benzyl)pyrrolidine-2- carboxamide | (2S,4R)-1-((S)-3,3- dimethyl-2-(6-oxo-6- (4-(4-(4-(4-((E)-3- (pyridin-3- yl)acrylamido)butyl) piperidine-1- carbonyl)phenyl) piperazin-1- yl)hexanamido) butanoyl)-4- hydroxy-N-(4-(4- methylthiazol-5- yl)benzyl)pyrrolidine- 2-carboxamide |
| SIAI S631 004 | 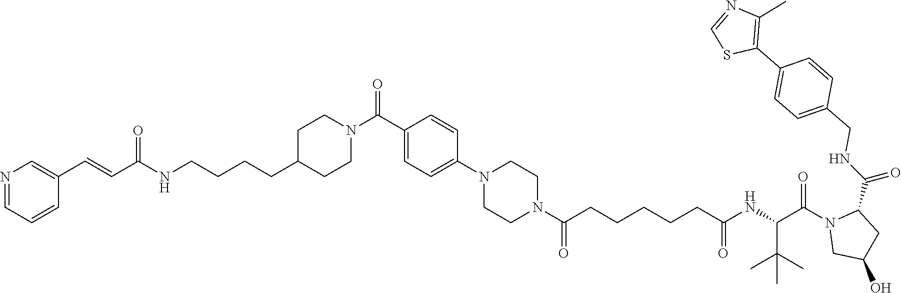 [SMILES: Cc1ncsc1-c1ccc(CNC(=O)[C@@H]2C[C@@H](O)CN2C(=O)[C@@H](NC(=O)CCCCCC(=O)N2CCN(c3ccc(C(=O)N4CCC(CCCCNC(=O)/C=C/c5cccnc5)CC4)cc3)CC2)C(C)(C)C)cc1] | (2S,4R)-1-((S)-3,3- dimethyl-2-(7-oxo-7-(4- (4-(4-(4-((E)-3- (pyridin-3- yl)acrylamido)butyl) piperidine-1- carbonyl)phenyl) piperazin-1- yl)heptanamido) butanoyl)-4- hydroxy-N-(4-(4- methylthiazol-5- yl)benzyl)pyrrolidine-2- carboxamide | (2S,4R)-1-((S)-3,3- dimethyl-2-(7-oxo-7- (4-(4-(4-(4-((E)-3- (pyridin-3- yl)acrylamido)butyl) piperidine-1- carbonyl)phenyl) piperazin-1- yl)heptanamido) butanoyl)- 4-hydroxy-N-(4-(4- methylthiazol-5- yl)benzyl)pyrrolidine- 2-carboxamide |
| SIAI S631 005 | 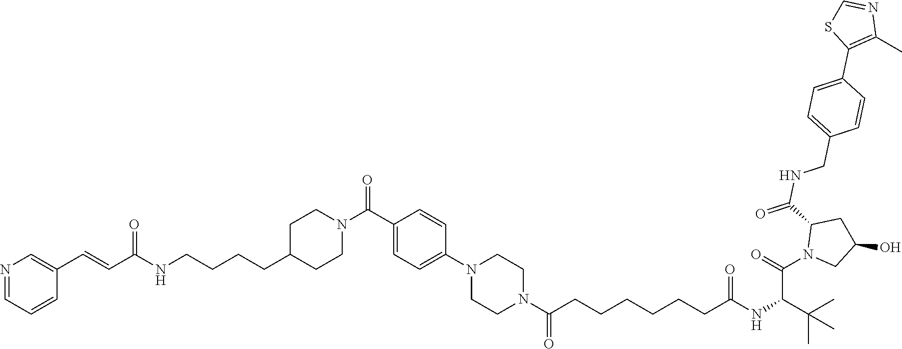 [SMILES: Cc1ncsc1-c1ccc(CNC(=O)[C@@H]2C[C@@H](O)CN2C(=O)[C@@H](NC(=O)CCCCCCC(=O)N2CCN(c3ccc(C(=O)N4CCC(CCCCNC(=O)/C=C/c5cccnc5)CC4)cc3)CC2)C(C)(C)C)cc1] | (2S,4R)-1-((S)-3,3- dimethyl-2-(8-oxo-8-(4- (4-(4-(4-((E)-3- (pyridin-3- yl)acrylamido)butyl) piperidine-1- carbonyl)phenyl) piperazin-1- yl)octanamido) butanoyl)-4- hydroxy-N-(4-(4- methylthiazol-5- yl)benzyl)pyrrolidine-2- carboxamide | (2S,4R)-1-((S)-3,3- dimethyl-2-(8-oxo-8- (4-(4-(4-(4-((E)-3- (pyridin-3- yl)acrylamido)butyl) piperidine-1- carbonyl)phenyl) piperazin-1- yl)octanamido) butanoyl)-4- hydroxy-N-(4-(4- methylthiazol-5- yl)benzyl)pyrrolidine- 2-carboxamide |
| SIAI S631 006 | 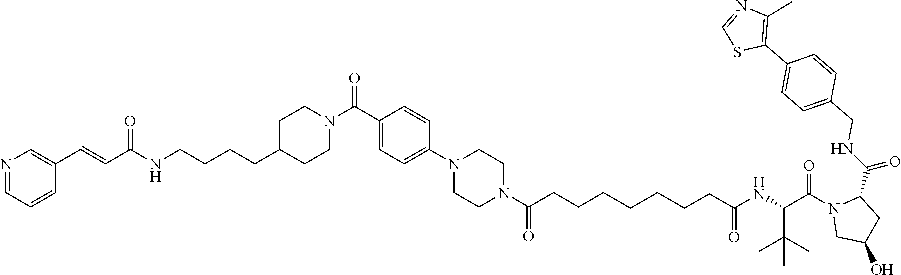 [SMILES: Cc1ncsc1-c1ccc(CNC(=O)[C@@H]2C[C@@H](O)CN2C(=O)[C@@H](NC(=O)CCCCCCCC(=O)N2CCN(c3ccc(C(=O)N4CCC(CCCCNC(=O)/C=C/c5cccnc5)CC4)cc3)CC2)C(C)(C)C)cc1] | (2S,4R)-1-((S)-3,3- dimethyl-2-(9-oxo-9-(4- (4-(4-(4-((E)-3- (pyridin-3- yl)acrylamido)butyl) piperidine-1- carbonyl)phenyl) piperazin-1- yl)nonanamido) butanoyl)-4- hydroxy-N-(4-(4- methylthiazol-5- yl)benzyl)pyrrolidine-2- carboxamide | (2S,4R)-1-((S)-3,3- dimethyl-2-(9-oxo-9- (4-(4-(4-(4-((E)-3- (pyridin-3- yl)acrylamido)butyl) piperidine-1- carbonyl)phenyl) piperazin-1- yl)nonanamido) butanoyl)- 4-hydroxy-N-(4-(4- methylthiazol-5- yl)benzyl)pyrrolidine- 2-carboxamide |
| SIAI S631 007 | 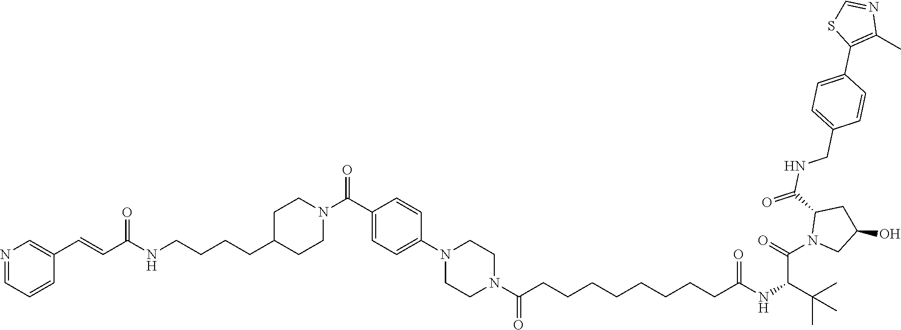 [SMILES: Cc1ncsc1-c1ccc(CNC(=O)[C@@H]2C[C@@H](O)CN2C(=O)[C@@H](NC(=O)CCCCCCCCC(=O)N2CCN(c3ccc(C(=O)N4CCC(CCCCNC(=O)/C=C/c5cccnc5)CC4)cc3)CC2)C(C)(C)C)cc1] | (2S,4R)-1-((S)-3,3- dimethyl-2-(10-oxo-10- (4-(4-(4-(4-((E)-3- (pyridin-3- yl)acrylamido)butyl) piperidine-1- carbonyl)phenyl) piperazin-1- yl)decanamido) butanoyl)-4- hydroxy-N-(4-(4- methylthiazol-5- yl)benzyl)pyrrolidine-2- carboxamide | (2S,4R)-1-((S)-3,3- dimethyl-2- (10-oxo-10- (4-(4-(4-(4-((E)-3- (pyridin-3- yl)acrylamido)butyl) piperidine-1- carbonyl)phenyl) piperazin-1- yl)decanamido) butanoyl)-4- hydroxy-N-(4-(4- methylthiazol-5- yl)benzyl)pyrrolidine- 2-carboxamide |
| SIAI S631 008 | 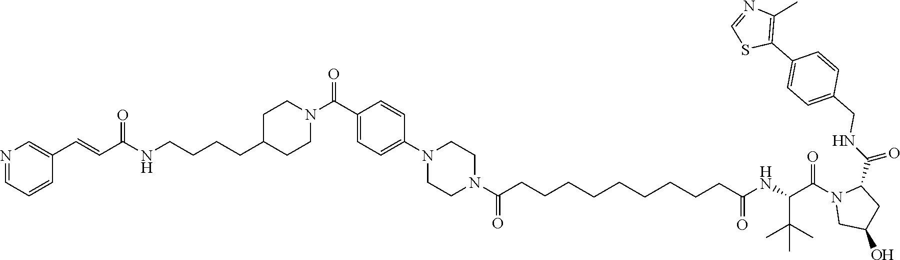 [SMILES: Cc1ncsc1-c1ccc(CNC(=O)[C@@H]2C[C@@H](O)CN2C(=O)[C@@H](NC(=O)CCCCCCCCCC(=O)N2CCN(c3ccc(C(=O)N4CCC(CCCCNC(=O)/C=C/c5cccnc5)CC4)cc3)CC2)C(C)(C)C)cc1] | (2S,4R)-1-((S)-3,3- dimethyl-2-(11-oxo-11- (4-(4-(4-(4-((E)-3- (pyridin-3- yl)acrylamido)butyl) piperidine-1- carbonyl)phenyl) piperazin-1- yl)undecanamido) butanoyl)-4- hydroxy-N-(4-(4- methylthiazol-5- yl)benzyl)pyrrolidine-2- carboxamide | (2S,4R)-1-((S)-3,3- dimethyl-2-(11-oxo-11- (4-(4-(4-(4-((E)-3- (pyridin-3- yl)acrylamido)butyl) piperidine-1- carbonyl)phenyl) piperazin-1- yl)undecanamido) butanoyl)-4- hydroxy-N-(4- (4-methylthiazol-5- yl)benzyl)pyrrolidine- 2-carboxamide |
| SIAI S631 010 | 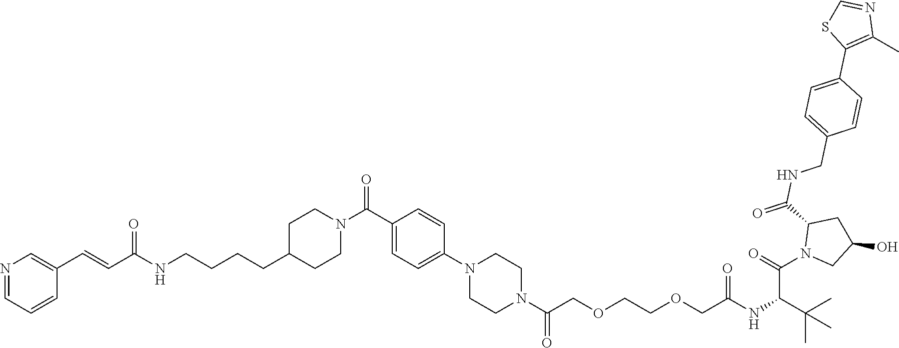 [SMILES: Cc1ncsc1-c1ccc(CNC(=O)[C@@H]2C[C@@H](O)CN2C(=O)[C@@H](NC(=O)COCCOCC(=O)N2CCN(c3ccc(C(=O)N4CCC(CCCCNC(=O)/C=C/c5cccnc5)CC4)cc3)CC2)C(C)(C)C)cc1] | (2S,4R)-1-((S)-3,3- dimethyl-2-(2-(2-(2- oxo-2-(4-(4-(4-(4-((E)- 3-(pyridin-3- yl)acrylamido)butyl) piperidine-1- carbonyl)phenyl) piperazin-1- yl)ethoxy)ethoxy) acetamido)butanoyl)- 4-hydroxy-N-(4-(4- methylthiazol-5- yl)benzyl)pyrrolidine- 2-carboxamide | (2S,4R)-1-((S)-3,3- dimethyl-2-(2-(2-(2- ox0-2-(4-(4-(4-(4-((E)- 3-(pyridin-3- yl)acrylamido)butyl) piperidine-1- carbonyl)phenyl) piperazin-1- yl)ethoxy)ethoxy) acetamido)butanoyl)- 4-hydroxy-N-(4-(4- methylthiazol-5- yl)benzyl)pyrrolidine- 2-carboxamide |
| SIAI S631 011 | 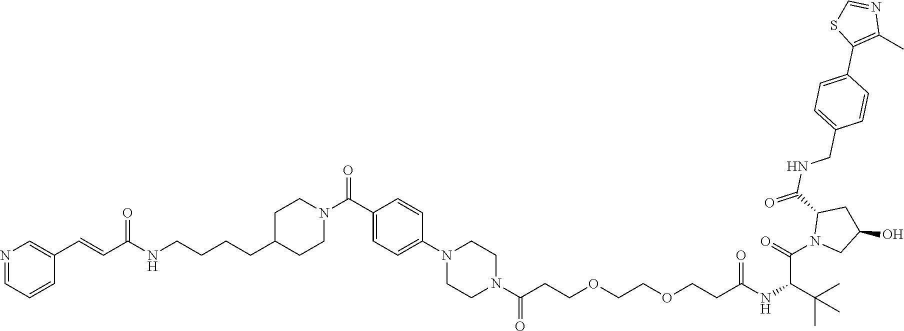 [SMILES: Cc1ncsc1-c1ccc(CNC(=O)[C@@H]2C[C@@H](O)CN2C(=O)[C@@H](NC(=O)CCOCCOCCC(=O)N2CCN(c3ccc(C(=O)N4CCC(CCCCNC(=O)/C=C/c5cccnc5)CC4)cc3)CC2)C(C)(C)C)cc1] | (2S,4R)-1-((S)-3,3- dimethyl-2-(3-(2-(3- ox0-3-(4-(4-(4-(4-((E)- 3-(pyridin-3- yl)acrylamido)butyl) piperidine-1- carbonyl)phenyl) piperazin-1- yl)propoxy)ethoxy) propanamido) butanoyl)-4- hydroxy-N-(4-(4- methylthiazol-5- yl)benzyl) pyrrolidine-2- carboxamide | (2S,4R)-1-((S)-3,3- dimethyl-2-(3-(2-(3- oxo-3-(4-(4-(4-(4-((E)- 3-(pyridin-3- yl)acrylamido)butyl) piperidine-1- carbonyl)phenyl) piperazin-1- yl)propoxy)ethoxy) propanamido) butanoyl)-4- hydroxy-N-(4-(4- methylthiazol-5- yl)benzyl)pyrrolidine- 2-carboxamide |
| SIAI S631 012 | 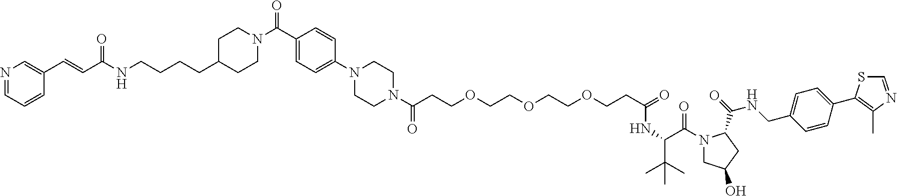 [SMILES: Cc1ncsc1-c1ccc(CNC(=O)[C@@H]2C[C@@H](O)CN2C(=O)[C@@H](NC(=O)CCOCCOCCOCCC(=O)N2CCN(c3ccc(C(=O)N4CCC(CCCCNC(=O)/C=C/c5cccnc5)CC4)cc3)CC2)C(C)(C)C)cc1] | (2S,4R)-1-((S)-2-(tert- butyl)-4,16-dioxo- 16-(4-(4-(4-(4-((E)- 3-(pyridin-3- yl)acrylamido)butyl) piperidine-1- carbonyl)phenyl) piperazin-1-yl)-7,10,13- trioxa-3- azahexadecanoyl)-4- hydroxy-N-(4-(4- methylthiazol-5- | (2S,4R)-1-((S)-2-(tert- butyl)-4,16-dioxo-16- (4-(4-(4-(4-((E)-3- (pyridin-3- yl)acrylamido)butyl) piperidine-1- carbonyl)phenyl) piperazin-1-yl)- 7,10,13-trioxa-3- azahexadecanoyl)-4- hydroxy-N-(4-(4- methylthiazol-5- |
| | | yl)benzyl)pyrrolidine-2- | yl)benzyl)pyrrolidine- |
| | | carboxamide | 2-carboxamide |
| SIAI S631 013 | 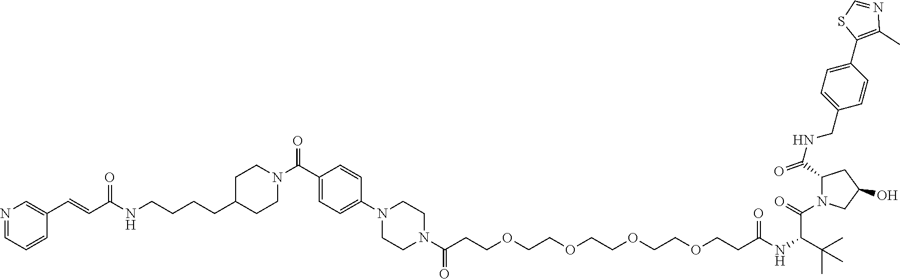 [SMILES: Cc1ncsc1-c1ccc(CNC(=O)[C@@H]2C[C@@H](O)CN2C(=O)[C@@H](NC(=O)CCOCCOCCOCCOCCC(=O)N2CCN(c3ccc(C(=O)N4CCC(CCCCNC(=O)/C=C/c5cccnc5)CC4)cc3)CC2)C(C)(C)C)cc1] | (2S,4R)-1-((S)-2-(tert- butyl)-4,19-dioxo- 19-(4-(4-(4-(4-((E)- 3-(pyridin-3- yl)acrylamido)butyl) piperidine-1- carbonyl)phenyl) piperazin-1-yl)- 7,10,13,16- tetraoxa-3- azanonadecanoyl)-4- hydroxy-N-(4-(4- methylthiazol-5- yl)benzyl)pyrrolidine- 2-carboxamide | (2S,4R)-1-((S)-2-(tert- butyl)-4,19-dioxo-19- (4-(4-(4-(4-((E)-3- (pyridin-3- yl)acrylamido)butyl) piperidine-1- carbonyl)phenyl) piperazin-1-yl)- 7,10,13,16- tetraoxa-3- azanonadecanoyl)-4- hydroxy-N-(4-(4- methylthiazol-5- yl)benzyl)pyrrolidine- 2-carboxamide |
| SIAI S631 014 | 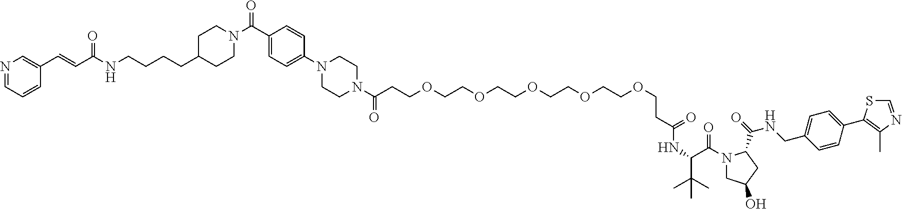 [SMILES: Cc1ncsc1-c1ccc(CNC(=O)[C@@H]2C[C@@H](O)CN2C(=O)[C@@H](NC(=O)CCOCCOCCOCCOCCOCCC(=O)N2CCN(c3ccc(C(=O)N4CCC(CCCCNC(=O)/C=C/c5cccnc5)CC4)cc3)CC2)C(C)(C)C)cc1] | (2S,4R)-1-((S)-2-(tert- butyl)-4,22-dioxo-22-(4- (4-(4-(4-((E)-3- (pyridin-3- yl)acrylamido)butyl) piperidine-1- carbonyl)phenyl) piperazin-1-yl)- 7,10,13,16,19- pentaoxa-3- azadocosanoyl)-4- hydroxy-N-(4-(4- methylthiazol-5- | (2S,4R)-1-((S)-2-(tert- butyl)-4,22-dioxo-22- (4-(4-(4-(4-((E)-3- (pyridin-3- yl)acrylamido)butyl) piperidine-1- carbonyl)phenyl) piperazin-1-yl)- 7,10,13,16,19- pentaoxa-3- azadocosanoyl)-4- hydroxy-N-(4-(4- methylthiazol-5- |
| | | yl)benzyl)pyrrolidine-2- | yl)benzyl)pyrrolidine- |
| | | carboxamide | 2-carboxamide |
| SIAI S630 008 | 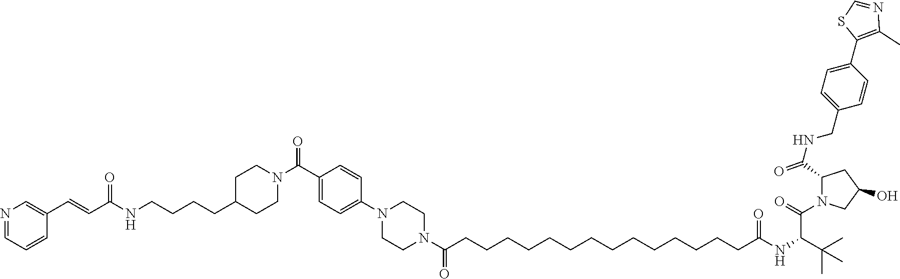 [SMILES: Cc1ncsc1-c1ccc(CNC(=O)[C@@H]2C[C@@H](O)CN2C(=O)[C@@H](NC(=O)CCCCCCCCCCCCCCC(=O)N2CCN(c3ccc(C(=O)N4CCC(CCCCNC(=O)/C=C/c5cccnc5)CC4)cc3)CC2)C(C)(C)C)cc1] | (2S,4R)-1-((S)-3,3- dimethyl-2-(16-oxo-16- (4-(4-(4-(4-((E)-3- (pyridin-3- yl)acrylamido)butyl) piperidine-1- carbonyl)phenyl) piperazin-1- yl)hexadecanamido) butanoyl)-4-hydroxy- N-(4-(4-methylthiazol- 5-yl)benzyl) pyrrolidine-2- carboxamide | (2S,4R)-1-((S)-3,3- dimethyl-2-(16-oxo- 16-(4-(4-(4-(4- ((E)-3-(pyridin-3- yl)acrylamido)butyl) piperidine-1- carbonyl)phenyl) piperazin-1- yl)hexadecanamido) butanoyl)-4- hydroxy-N- (4-(4-methylthiazol-5- yl)benzyl)pyrrolidine- 2-carboxamide |
| SIAI S631 020 | 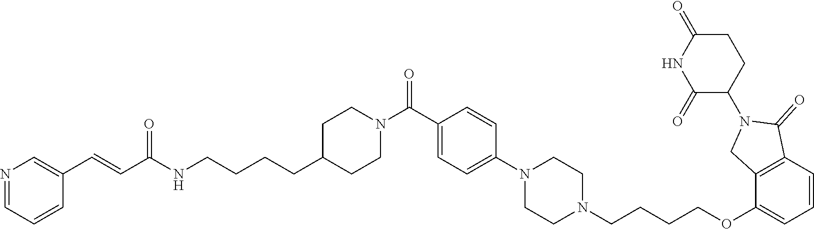 [SMILES: O=C(/C=C/c1cccnc1)NCCCCC1CCN(C(=O)c2ccc(N3CCN(CCCCOc4cccc5c4CN(C4CCC(=O)NC4=O)C5=O)CC3)cc2)CC1] | (E)-N-(4-(1-(4-(4-(4- ((2-(2,6-dioxopiperidin- 3-yl)-1-oxoisoindolin-4- yl)oxy)butyl)piperazin- 1-yl)benzoyl)piperidin- 4-yl)butyl)-3-(pyridin-3- yl)acrylamide | (E)-N-(4-(1-(4-(4-(4- ((2-(2,6- dioxopiperidin-3-yl)- 1-oxoisoindolin-4- yl)oxy)butyl)piperazin- 1-yl)benzoyl)piperidin- 4-yl)butyl)-3-(pyridin- 3-yl)acrylamide |
| SIAI S631 024 | 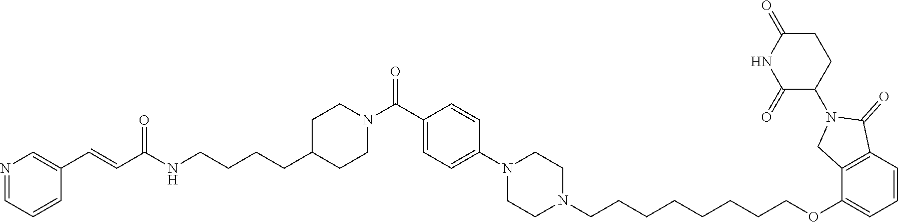 [SMILES: O=C(/C=C/c1cccnc1)NCCCCC1CCN(C(=O)c2ccc(N3CCN(CCCCCCCCOc4cccc5c4CN(C4CCC(=O)NC4=O)C5=O)CC3)cc2)CC1] | (E)-N-(4-(1-(4-(4-(8- ((2-(2,6-dioxopiperidin- 3-yl)-1-oxoisoindolin-4- yl)oxy)octyl)piperazin- 1-yl)benzoyl)piperidin- 4-yl)butyl)-3-(pyridin-3- yl)acrylamide | (E)-N-(4-(1-(4-(4-(8- ((2-(2,6- dioxopiperidin-3-yl)- 1-oxoisoindolin-4- yl)oxy)octyl)piperazin- 1-yl)benzoyl)piperidin- 4-yl)butyl)-3-(pyridin- 3-yl)acrylamide |
| SIAI S631 045 | 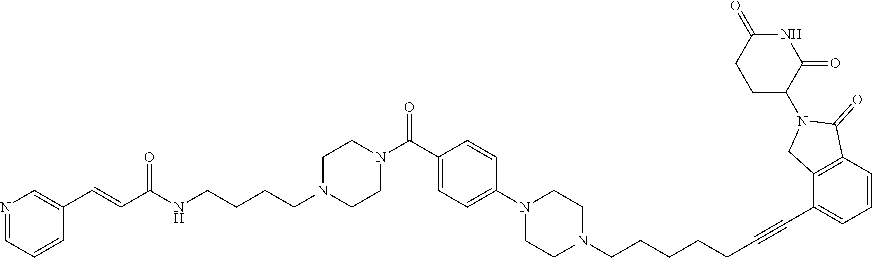 [SMILES: O=C(/C=C/c1cccnc1)NCCCCN1CCN(C(=O)c2ccc(N3CCN(CCCCCC#Cc4cccc5c4CN(C4CCC(=O)NC4=O)C5=O)CC3)cc2)CC1] | (E)-N-(4-(4-(4-(4-(7-(2- (2,6-dioxopiperidin-3- yl)-1-oxoisoindolin-4- yl)hept-6-yn-1- yl)piperazin-1- yl)benzoyl)piperazin-1- yl)butyl)-3-(pyridin-3- yl)acrylamide | (E)-N-(4-(4-(4-(4-(7- (2-(2,6-dioxopiperidin- 3-yl)-1-oxoisoindolin- 4-yl)hept-6-yn-1- yl)piperazin-1- yl)benzoyl)piperazin-1- yl)butyl)-3-(pyridin-3- yl)acrylamide |
| SIAI S631 068 | 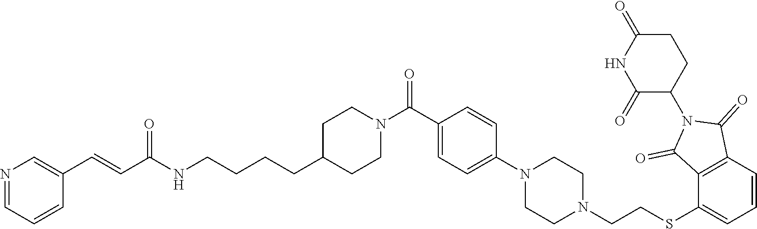 [SMILES: O=C(/C=C/c1cccnc1)NCCCCC1CCN(C(=O)c2ccc(N3CCN(CCSc4cccc5c4C(=O)N(C4CCC(=O)NC4=O)C5=O)CC3)cc2)CC1] | (E)-N-(4-(1-(4-(4-(2- ((2-(2,6-dioxopiperidin- 3-yl)-1,3- dioxoisoindolin-4- yl)thio)ethyl)piperazin- 1-yl)benzoyl)piperidin- 4-yl)butyl)-3-(pyridin-3- yl)acrylamide | (E)-N-(4-(1-(4-(4-(2- ((2-(2,6- dioxopiperidin-3-yl)- 1,3-dioxoisoindolin-4- yl)thio)ethyl)piperazin- 1-yl)benzoyl)piperidin- 4-yl)butyl)-3-(pyridin- 3-yl)acrylamide |
| SIAI S631 069 | 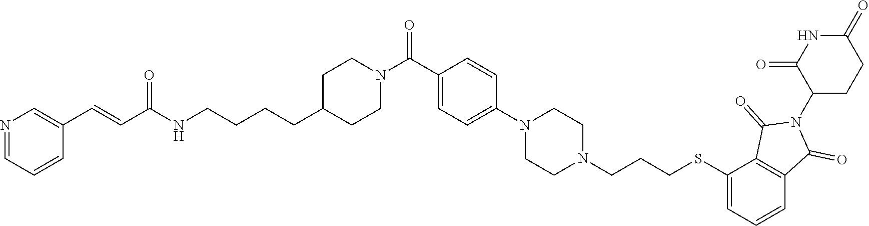 [SMILES: O=C(/C=C/c1cccnc1)NCCCCC1CCN(C(=O)c2ccc(N3CCN(CCCSc4cccc5c4C(=O)N(C4CCC(=O)NC4=O)C5=O)CC3)cc2)CC1] | (E)-N-(4-(1-(4-(4-(3- ((2-(2,6-dioxopiperidin- 3-yl)-1,3- dioxoisoindolin-4- yl)thio)propyl) piperazin- 1-yl)benzoyl)piperidin- 4-yl)butyl)-3-(pyridin-3- yl)acrylamide | (E)-N-(4-(1-(4-(4-(3- ((2-(2,6- dioxopiperidin-3-yl)- 1,3-dioxoisoindolin-4- yl)thio)propyl) piperazin-1- yl)benzoyl)piperidin- 4-yl)butyl)-3- (pyridin-3- yl)acrylamide |
| SIAI S631 070 | 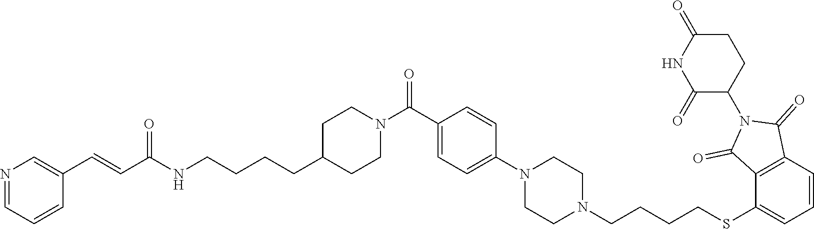 [SMILES: O=C(/C=C/c1cccnc1)NCCCCC1CCN(C(=O)c2ccc(N3CCN(CCCCSc4cccc5c4C(=O)N(C4CCC(=O)NC4=O)C5=O)CC3)cc2)CC1] | (E)-N-(4-(1-(4-(4-(4- ((2-(2,6-dioxopiperidin- 3-yl)-1,3- dioxoisoindolin-4- yl)thio)butyl)piperazin- 1-yl)benzoyl)piperidin- 4-yl)butyl)-3-(pyridin-3- yl)acrylamide | (E)-N-(4-(1-(4-(4-(4- ((2-(2,6- dioxopiperidin-3-yl)- 1,3-dioxoisoindolin-4- yl)thio)butyl)piperazin- 1-yl)benzoyl)piperidin- 4-yl)butyl)-3-(pyridin- 3-yl)acrylamide |
| SIAI S631 071 | 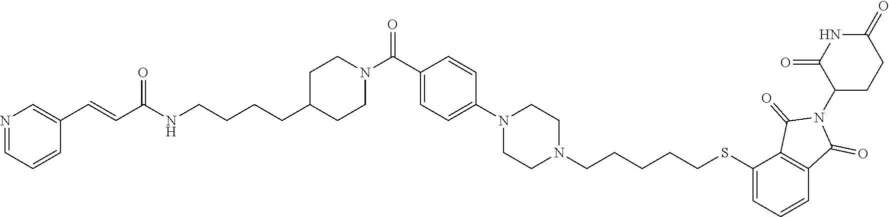 [SMILES: O=C(/C=C/c1cccnc1)NCCCCC1CCN(C(=O)c2ccc(N3CCN(CCCCCSc4cccc5c4C(=O)N(C4CCC(=O)NC4=O)C5=O)CC3)cc2)CC1] | (E)-N-(4-(1-(4-(4-(5- ((2-(2,6-dioxopiperidin- 3-yl)-1,3- dioxoisoindolin-4- yl)thio)pentyl)piperazin- 1-yl)benzoyl)piperidin- 4-yl)butyl)-3-(pyridin-3- yl)acrylamide | (E)-N-(4-(1-(4-(4-(5- ((2-(2,6- dioxopiperidin-3-yl)- 1,3-dioxoisoindolin-4- yl)thio)pentyl) piperazin-1- yl)benzoyl)piperidin-4- yl)butyl)-3-(pyridin-3- yl)acrylamide |
| SIAI S631 072 | 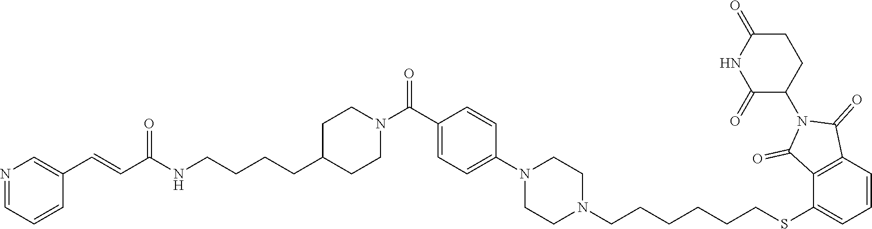 [SMILES: O=C(/C=C/c1cccnc1)NCCCCC1CCN(C(=O)c2ccc(N3CCN(CCCCCCSc4cccc5c4C(=O)N(C4CCC(=O)NC4=O)C5=O)CC3)cc2)CC1] | (E)-N-(4-(1-(4-(4-(6- ((2-(2,6-dioxopiperidin- 3-yl)-1,3- dioxoisoindolin-4- yl)thio)hexyl)piperazin- 1-yl)benzoyl)piperidin- 4-yl)butyl)-3-(pyridin-3- yl)acrylamide | (E)-N-(4-(1-(4-(4-(6- ((2-(2,6- dioxopiperidin-3-yl)- 1,3-dioxoisoindolin-4- yl)thio)hexyl) piperazin-1- yl)benzoyl)piperidin-4- yl)butyl)-3-(pyridin-3- yl)acrylamide |
| SIAI S631 078 | 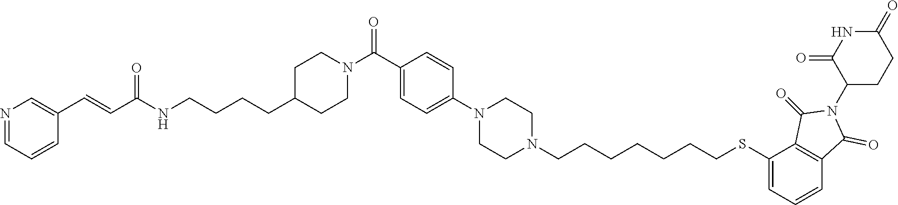 [SMILES: O=C(/C=C/c1cccnc1)NCCCCC1CCN(C(=O)c2ccc(N3CCN(CCCCCCCSc4cccc5c4C(=O)N(C4CCC(=O)NC4=O)C5=O)CC3)cc2)CC1] | (E)-N-(4-(1-(4-(4-(7- ((2-(2,6-dioxopiperidin- 3-yl)-1,3- dioxoisoindolin-4- yl)thio)heptyl)piperazin- 1-yl)benzoyl)piperidin- 4-yl)butyl)-3-(pyridin-3- yl)acrylamide | (E)-N-(4-(1-(4-(4-(7- ((2-(2,6- dioxopiperidin-3-yl)- 1,3-dioxoisoindolin-4- yl)thio)heptyl) piperazin-1- yl)benzoyl)piperidin-4- yl)butyl)-3-(pyridin-3- yl)acrylamide |
| SIAI S631 073 | 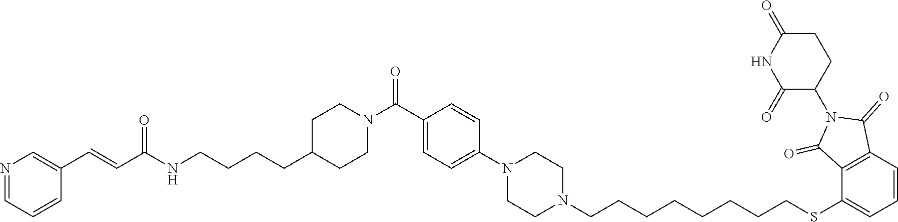 [SMILES: O=C(/C=C/c1cccnc1)NCCCCC1CCN(C(=O)c2ccc(N3CCN(CCCCCCCCSc4cccc5c4C(=O)N(C4CCC(=O)NC4=O)C5=O)CC3)cc2)CC1] | (E)-N-(4-(1-(4-(4-(8- ((2-(2,6-dioxopiperidin- 3-yl)-1,3- dioxoisoindolin-4- yl)thio)octyl)piperazin- 1-yl)benzoyl)piperidin- 4-yl)butyl)-3-(pyridin-3- yl)acrylamide | (E)-N-(4-(1-(4-(4-(8- ((2-(2,6- dioxopiperidin-3-yl)- 1,3-dioxoisoindolin-4- yl)thio)octyl)piperazin- 1-yl)benzoyl)piperidin- 4-yl)butyl)-3-(pyridin- 3-yl)acrylamide |
| SIAI S632 027 | 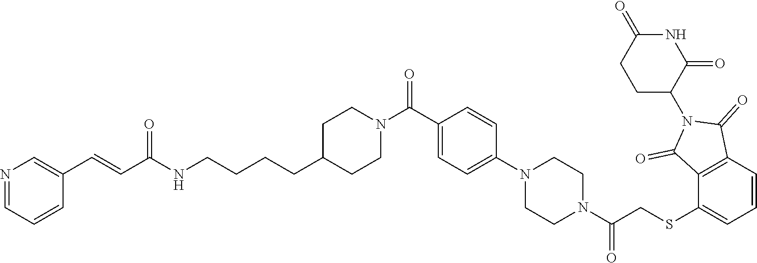 [SMILES: O=C(/C=C/c1cccnc1)NCCCCC1CCN(C(=O)c2ccc(N3CCN(C(=O)CSc4cccc5c4C(=O)N(C4CCC(=O)NC4=O)C5=O)CC3)cc2)CC1] | (E)-N-(4-(1-(4-(4-(2- ((2-(2,6-dioxopiperidin- 3-yl)-1,3- dioxoisoindolin-4- yl)thio)acetyl)piperazin- 1-yl)benzoyl)piperidin- 4-yl)butyl)-3-(pyridin-3- yl)acrylamide | (E)-N-(4-(1-(4-(4-(2- ((2-(2,6- dioxopiperidin-3-yl)- 1,3-dioxoisoindolin-4- yl)thio)acetyl) piperazin-1- yl)benzoyl)piperidin-4- yl)butyl)-3-(pyridin-3- yl)acrylamide |
| SIAI S632 028 | 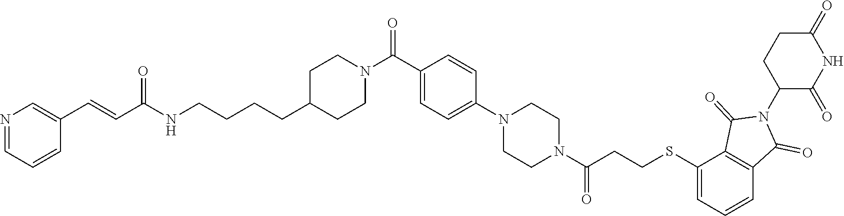 [SMILES: O=C(/C=C/c1cccnc1)NCCCCC1CCN(C(=O)c2ccc(N3CCN(C(=O)CCSc4cccc5c4C(=O)N(C4CCC(=O)NC4=O)C5=O)CC3)cc2)CC1] | (E)-N-(4-(1-(4-(4-(3- ((2-(2,6-dioxopiperidin- 3-yl)-1,3- dioxoisoindolin-4- yl)thio)propanoyl) piperazin-1- yl)benzoyl)piperidin-4- yl)butyl)-3-(pyridin-3- yl)acrylamide | (E)-N-(4-(1-(4-(4-(3- ((2-(2,6- dioxopiperidin-3-yl)- 1,3-dioxoisoindolin-4- yl)thio)propanoyl) piperazin-1- yl)benzoyl)piperidin-4- yl)butyl)-3-(pyridin-3- yl)acrylamide |
| SIAI S632 029 | 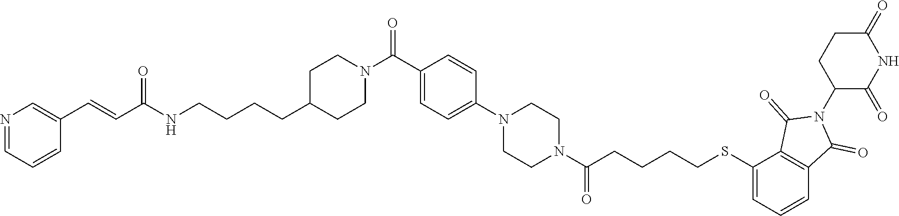 [SMILES: O=C(/C=C/c1cccnc1)NCCCCC1CCN(C(=O)c2ccc(N3CCN(C(=O)CCCCSc4cccc5c4C(=O)N(C4CCC(=O)NC4=O)C5=O)CC3)cc2)CC1] | (E)-N-(4-(1-(4-(4-(5- ((2-(2,6-dioxopiperidin- 3-yl)-1,3- dioxoisoindolin-4- yl)thio)pentanoyl) piperazin-1- yl)benzoyl)piperidin-4- yl)butyl)-3-(pyridin-3- yl)acrylamide | (E)-N-(4-(1-(4-(4-(5- ((2-(2,6- dioxopiperidin-3-yl)- 1,3-dioxoisoindolin-4- yl)thio)pentanoyl) piperazin-1- yl)benzoyl)piperidin-4- yl)butyl)-3-(pyridin-3- yl)acrylamide |
| SIAI S632 030 | 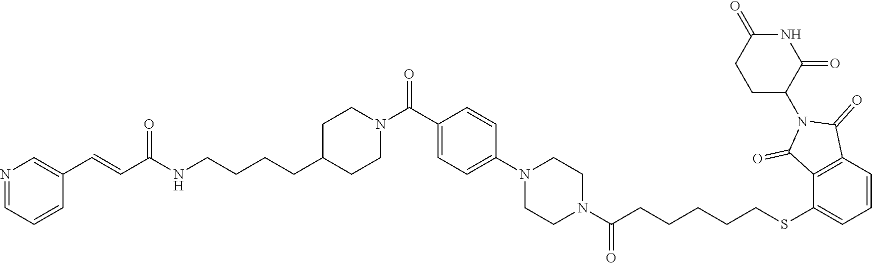 [SMILES: O=C(/C=C/c1cccnc1)NCCCCC1CCN(C(=O)c2ccc(N3CCN(C(=O)CCCCCSc4cccc5c4C(=O)N(C4CCC(=O)NC4=O)C5=O)CC3)cc2)CC1] | (E)-N-(4-(1-(4-(4-(6- ((2-(2,6-dioxopiperidin- 3-yl)-1,3- dioxoisoindolin-4- yl)thio)hexanoyl) piperazin-1- yl)benzoyl)piperidin-4- yl)butyl)-3-(pyridin-3- yl)acrylamide | (E)-N-(4-(1-(4-(4-(6- ((2-(2,6- dioxopiperidin-3-yl)- 1,3-dioxoisoindolin-4- yl)thio)hexanoyl) piperazin-1- yl)benzoyl)piperidin-4- yl)butyl)-3-(pyridin-3- yl)acrylamide |
| SIAI S632 031 | 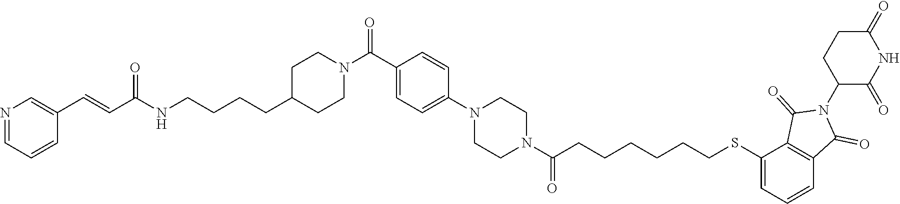 [SMILES: O=C(/C=C/c1cccnc1)NCCCCC1CCN(C(=O)c2ccc(N3CCN(C(=O)CCCCCCSc4cccc5c4C(=O)N(C4CCC(=O)NC4=O)C5=O)CC3)cc2)CC1] | (E)-N-(4-(1-(4-(4-(7- ((2-(2,6-dioxopiperidin- 3-yl)-1,3- dioxoisoindolin-4- yl)thio)heptanoyl) piperazin-1- yl)benzoyl)piperidin-4- yl)butyl)-3-(pyridin-3- yl)acrylamide | (E)-N-(4-(1-(4-(4-(7- ((2-(2,6- dioxopiperidin-3-yl)- 1,3-dioxoisoindolin-4- yl)thio)heptanoyl) piperazin-1- yl)benzoyl)piperidin-4- yl)butyl)-3-(pyridin-3- yl)acrylamide |
| SIAI S632 032 | 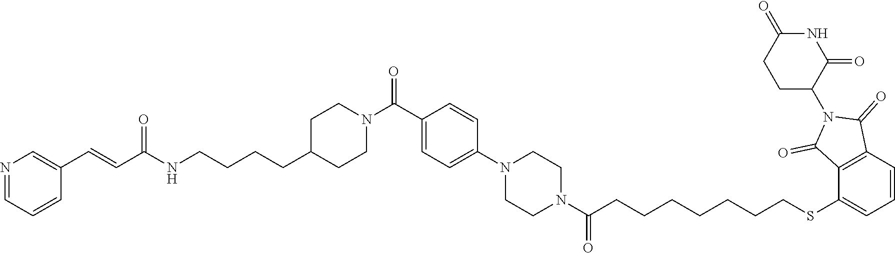 [SMILES: O=C(/C=C/c1cccnc1)NCCCCC1CCN(C(=O)c2ccc(N3CCN(C(=O)CCCCCCCSc4cccc5c4C(=O)N(C4CCC(=O)NC4=O)C5=O)CC3)cc2)CC1] | (E)-N-(4-(1-(4-(4-(8- ((2-(2,6-dioxopiperidin- 3-yl)-1,3- dioxoisoindolin-4- yl)thio)octanoyl) piperazin-1- yl)benzoyl)piperidin-4- yl)butyl)-3-(pyridin-3- yl)acrylamide | (E)-N-(4-(1-(4-(4-(8- ((2-(2,6- dioxopiperidin-3-yl)- 1,3-dioxoisoindolin-4- yl)thio)octanoyl) piperazin-1- yl)benzoyl)piperidin-4- yl)butyl)-3-(pyridin-3- yl)acrylamide |
| SIAI S632 033 | 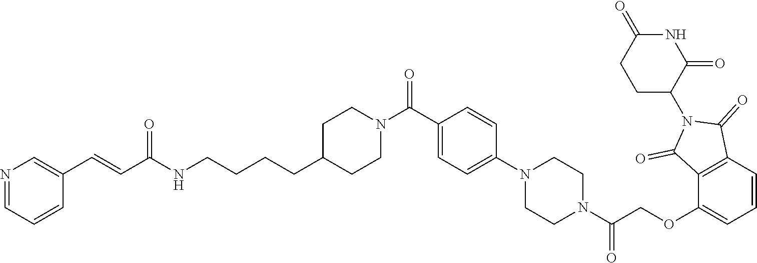 [SMILES: O=C(/C=C/c1cccnc1)NCCCCC1CCN(C(=O)c2ccc(N3CCN(C(=O)COc4cccc5c4C(=O)N(C4CCC(=O)NC4=O)C5=O)CC3)cc2)CC1] | (E)-N-(4-(1-(4-(4-(2- ((2-(2,6-dioxopiperidin- 3-yl)-1,3- dioxoisoindolin-4- yl)oxy)acetyl)piperazin- 1-yl)benzoyl)piperidin- 4-yl)butyl)-3-(pyridin-3- yl)acrylamide | (E)-N-(4-(1-(4-(4-(2- ((2-(2,6- dioxopiperidin-3-yl)- 1,3-dioxoisoindolin-4- yl)oxy)acetyl) piperazin-1- yl)benzoyl)piperidin-4- yl)butyl)-3-(pyridin-3- yl)acrylamide |
| SIAI S632 034 | 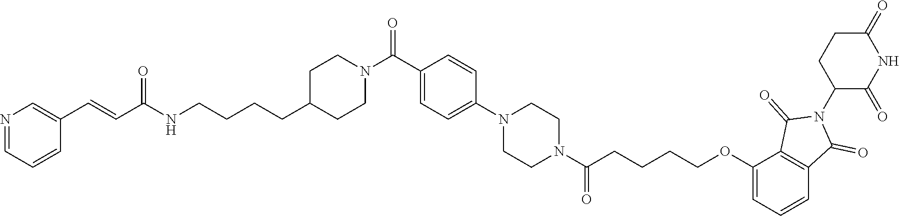 [SMILES: O=C(/C=C/c1cccnc1)NCCCCC1CCN(C(=O)c2ccc(N3CCN(C(=O)CCCCOc4cccc5c4C(=O)N(C4CCC(=O)NC4=O)C5=O)CC3)cc2)CC1] | (E)-N-(4-(1-(4-(4-(5- ((2-(2,6-dioxopiperidin- 3-yl)-1,3- dioxoisoindolin-4- yl)oxy)pentanoyl) piperazin-1- yl)benzoyl)piperidin-4- yl)butyl)-3-(pyridin-3- yl)acrylamide | (E)-N-(4-(1-(4-(4-(5- ((2-(2,6- dioxopiperidin-3-yl)- 1,3-dioxoisoindolin-4- yl)oxy)pentanoyl) piperazin-1- yl)benzoyl)piperidin-4- yl)butyl)-3-(pyridin-3- yl)acrylamide |
| SIAI S632 035 | 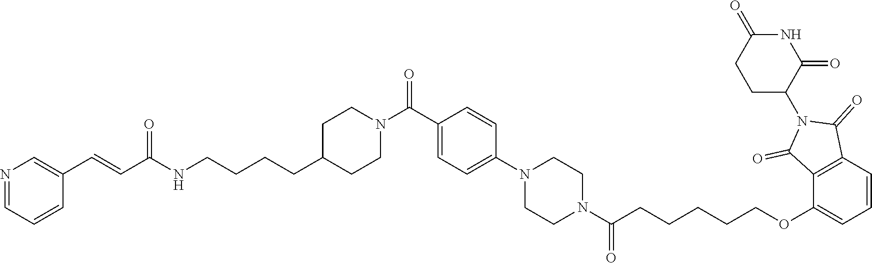 [SMILES: O=C(/C=C/c1cccnc1)NCCCCC1CCN(C(=O)c2ccc(N3CCN(C(=O)CCCCCOc4cccc5c4C(=O)N(C4CCC(=O)NC4=O)C5=O)CC3)cc2)CC1] | (E)-N-(4-(1-(4-(4-(6- ((2-(2,6-dioxopiperidin- 3-yl)-1,3- dioxoisoindolin-4- yl)oxy)hexanoyl) piperazin-1- yl)benzoyl)piperidin-4- yl)butyl)-3-(pyridin-3- yl)acrylamide | (E)-N-(4-(1-(4-(4-(6- ((2-(2,6- dioxopiperidin-3-yl)- 1,3-dioxoisoindolin-4- yl)oxy)hexanoyl) piperazin-1- yl)benzoyl)piperidin-4- yl)butyl)-3-(pyridin-3- yl)acrylamide |
| SIAI S632 036 | 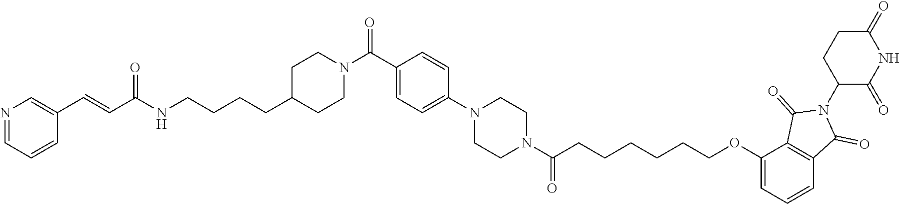 [SMILES: O=C(/C=C/c1cccnc1)NCCCCC1CCN(C(=O)c2ccc(N3CCN(C(=O)CCCCCCOc4cccc5c4C(=O)N(C4CCC(=O)NC4=O)C5=O)CC3)cc2)CC1] | (E)-N-(4-(1-(4-(4-(7- ((2-(2,6-dioxopiperidin- 3-yl)-1,3- dioxoisoindolin-4- yl)oxy)heptanoyl) piperazin-1- yl)benzoyl)piperidin-4- yl)butyl)-3-(pyridin-3- yl)acrylamide | (E)-N-(4-(1-(4-(4-(7- ((2-(2,6- dioxopiperidin-3-yl)- 1,3-dioxoisoindolin-4- yl)oxy)heptanoyl) piperazin-1- yl)benzoyl)piperidin-4- yl)butyl)-3-(pyridin-3- yl)acrylamide |
| SIAI S631 139 | 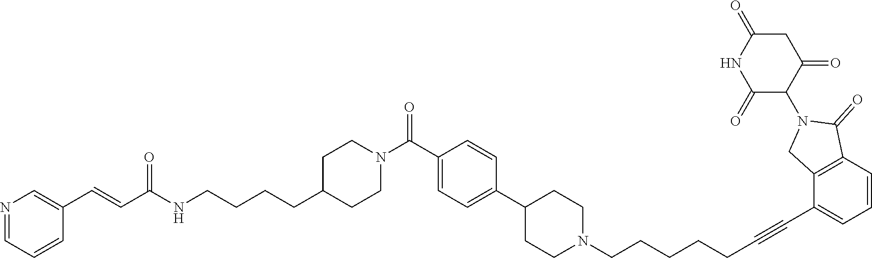 [SMILES: O=C(/C=C/c1cccnc1)NCCCCC1CCN(C(=O)c2ccc(C3CCN(CCCCCC#Cc4cccc5c4CN(C4C(=O)CC(=O)NC4=O)C5=O)CC3)cc2)CC1] | (E)-N-(4-(1-(4-(1-(7-(2- (2,6-dioxopiperidin-3- yl)-1-oxoisoindolin-4- yl)hept-6-yn-1- yl)piperidin-4- yl)benzoyl)piperidin-4- yl)butyl)-3-(pyridin-3- yl)acrylamide | (E)-N-(4-(1-(4-(1-(7- (2-(2,6-dioxopiperidin- 3-yl)-1-oxoisoindolin- 4-yl)hept-6-yn-1- yl)piperidin-4- yl)benzoyl)piperidin-4- yl)butyl)-3-(pyridin-3- yl)acrylamide |
| SIAI S631 140 | 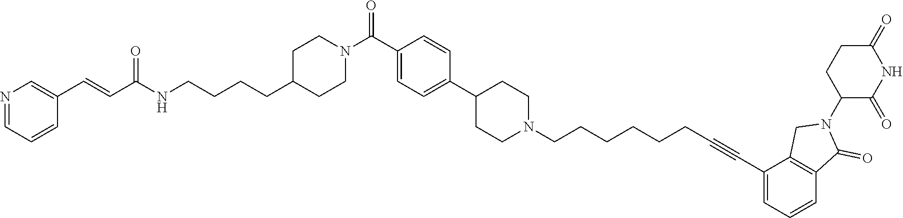 [SMILES: O=C(/C=C/c1cccnc1)NCCCCC1CCN(C(=O)c2ccc(C3CCN(CCCCCCC#Cc4cccc5c4CN(C4CCC(=O)NC4=O)C5=O)CC3)cc2)CC1] | (E)-N-(4-(1-(4-(1-(8-(2- (2,6-dioxopiperidin-3- yl)-1-oxoisoindolin-4- yl)oct-7-yn-1- yl)piperidin-4- yl)benzoyl)piperidin-4- yl)butyl)-3-(pyridin-3- yl)acrylamide | (E)-N-(4-(1-(4-(1-(8- (2-(2,6-dioxopiperidin- 3-yl)-1-oxoisoindolin- 4-yl)oct-7-yn-1- yl)piperidin-4- yl)benzoyl)piperidin-4- yl)butyl)-3-(pyridin-3- yl)acrylamide |
| SIAI S631 141 | 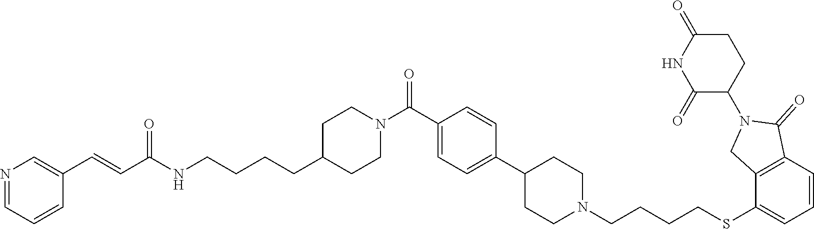 [SMILES: O=C(/C=C/c1cccnc1)NCCCCC1CCN(C(=O)c2ccc(C3CCN(CCCCSc4cccc5c4CN(C4CCC(=O)NC4=O)C5=O)CC3)cc2)CC1] | (E)-N-(4-(1-(4-(1-(4- ((2-(2,6-dioxopiperidin- 3-yl)-1-oxoisoindolin-4- yl)thio)butyl)piperidin- 4-yl)benzoyl)piperidin- 4-yl)butyl)-3-(pyridin-3- yl)acrylamide | (E)-N-(4-(1-(4-(1-(4- ((2-(2,6- dioxopiperidin-3-yl)-1- oxoisoindolin-4- yl)thio)butyl)piperidin- 4-yl)benzoyl)piperidin- 4-yl)butyl)-3-(pyridin- 3-yl)acrylamide |
| SIAI S632 005 | 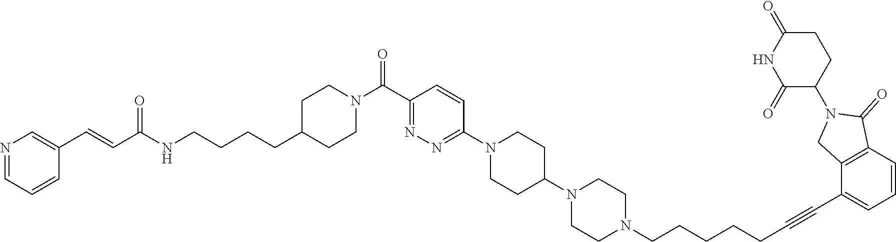 [SMILES: O=C(/C=C/c1cccnc1)NCCCCC1CCN(C(=O)c2ccc(N3CCC(N4CCN(CCCCCC#Cc5cccc6c5CN(C5CCC(=O)NC5=O)C6=O)CC4)CC3)nn2)CC1] | (E)-N-(4-(1-(6-(4-(4-(7- (2-(2,6-dioxopiperidin- 3-yl)-1-oxoisoindolin- 4-yl)hept-6-yn-1- yl)piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (7-(2-(2,6- dioxopiperidin-3-yl)- 1-oxoisoindolin-4- yl)hept-6-yn-1- yl)piperazin-1- yl)piperidin-1- yl)pyridazine-3- carbonyl)piperidin-4- yl)butyl)-3-(pyridin-3- yl)acrylamide |
| SIAI S632 006 | 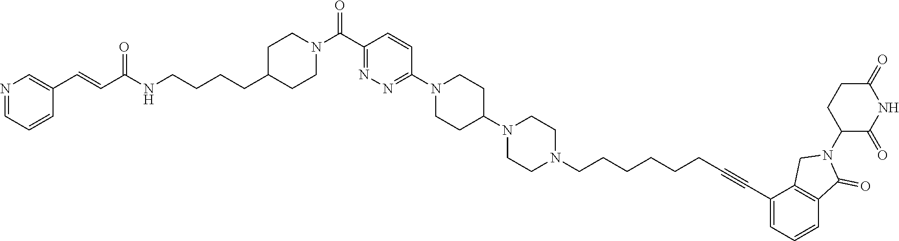 [SMILES: O=C(/C=C/c1cccnc1)NCCCCC1CCN(C(=O)c2ccc(N3CCC(N4CCN(CCCCCCC#Cc5cccc6c5CN(C5CCC(=O)NC5=O)C6=O)CC4)CC3)nn2)CC1] | (E)-N-(4-(1-(6-(4-(4-(8- (2-(2,6-dioxopiperidin- 3-yl)-1-oxoisoindolin- 4-yl)oct-7-yn-1- yl)piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (8-(2-(2,6- dioxopiperidin-3-yl)-1- oxoisoindolin-4-yl)oct- 7-yn-1-yl)piperazin-1- yl)piperidin-1- yl)pyridazine-3- carbonyl)piperidin-4- yl)butyl)-3-(pyridin-3- yl)acrylamide |
| SIAI S632 007 | 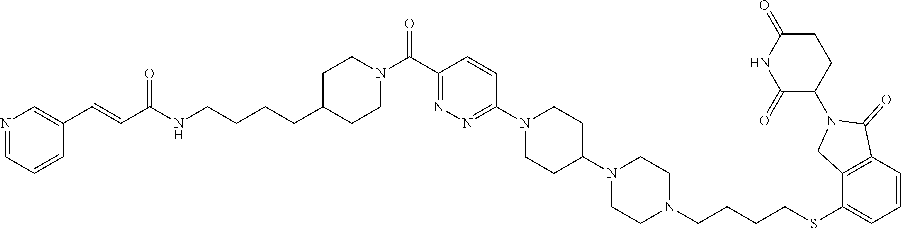 [SMILES: O=C(/C=C/c1cccnc1)NCCCCC1CCN(C(=O)c2ccc(N3CCC(N4CCN(CCCCSc5cccc6c5CN(C5CCC(=O)NC5=O)C6=O)CC4)CC3)nn2)CC1] | (E)-N-(4-(1-(6-(4-(4-(4- ((2-(2,6-dioxopiperidin- 3-yl)-1-oxoisoindolin-4- yl)thio)butyl)piperazin- 1-yl)piperidin-1- yl)pyridazine-3- carbonyl)piperidin-4- yl)butyl)-3-(pyridin-3- yl)acrylamide | (E)-N-(4-(1-(6-(4-(4- (4-((2-(2,6- dioxopiperidin-3-yl)- 1-oxoisoindolin-4- yl)thio)butyl) piperazin- 1-yl)piperidin-1- yl)pyridazine-3- carbonyl)piperidin-4- yl)butyl)-3-(pyridin- 3-yl)acrylamide |
| SIAI S632 010 | 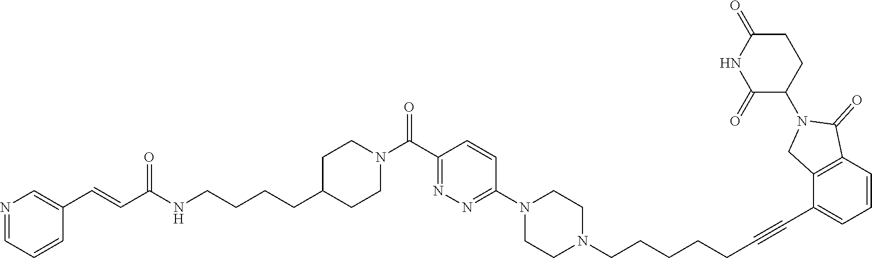 [SMILES: O=C(/C=C/c1cccnc1)NCCCCC1CCN(C(=O)c2ccc(N3CCN(CCCCCC#Cc4cccc5c4CN(C4CCC(=O)NC4=O)C5=O)CC3)nn2)CC1] | (E)-N-(4-(1-(6-(4-(7-(2- (2,6-dioxopiperidin-3- yl)-1-oxoisoindolin-4- yl)hept-6-yn-1- yl)piperazin-1- yl)pyridazine-3- carbonyl)piperidin-4- yl)butyl)-3-(pyridin-3- yl)acrylamide | (E)-N-(4-(1-(6-(4-(7- (2-(2,6-dioxopiperidin- 3-yl)-1-oxoisoindolin- 4-yl)hept-6-yn-1- yl)piperazin-1- yl)pyridazine-3- carbonyl)piperidin-4- yl)butyl)-3-(pyridin-3- yl)acrylamide |
| SIAI S632 011 | 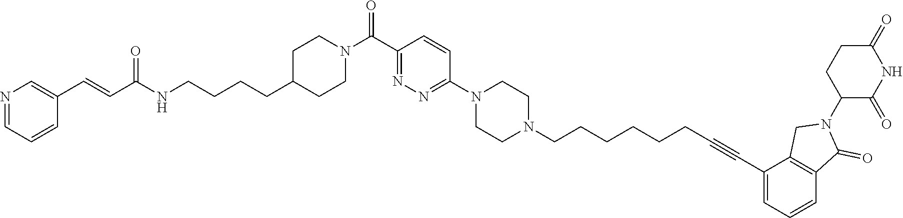 [SMILES: O=C(/C=C/c1cccnc1)NCCCCC1CCN(C(=O)c2ccc(N3CCN(CCCCCCC#Cc4cccc5c4CN(C4CCC(=O)NC4=O)C5=O)CC3)nn2)CC1] | (E)-N-(4-(1-(6-(4-(8-(2- (2,6-dioxopiperidin-3- yl)-1-oxoisoindolin-4- yl)oct-7-yn-1- yl)piperazin-1- yl)pyridazine-3- carbonyl)piperidin-4- yl)butyl)-3-(pyridin-3- yl)acrylamide | (E)-N-(4-(1-(6-(4-(8- (2-(2,6-dioxopiperidin- 3-yl)-1-oxoisoindolin- 4-yl)oct-7-yn-1- yl)piperazin-1- yl)pyridazine-3- carbonyl)piperidin-4- yl)butyl)-3-(pyridin-3- yl)acrylamide |
| SIAI S632 012 | 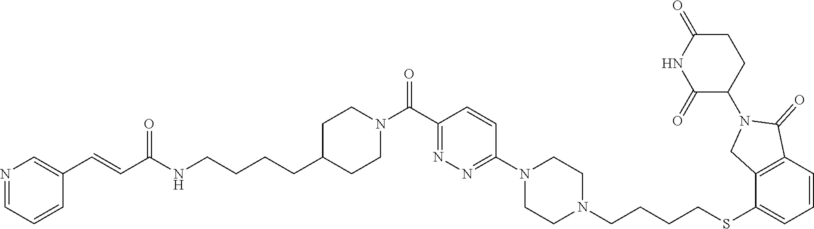 [SMILES: O=C(/C=C/c1cccnc1)NCCCCC1CCN(C(=O)c2ccc(N3CCN(CCCCSc4cccc5c4CN(C4CCC(=O)NC4=O)C5=O)CC3)nn2)CC1] | (E)-N-(4-(1-(6-(4-(4- ((2-(2,6-dioxopiperidin- 3-yl)-1-oxoisoindolin-4- yl)thio)butyl)piperazin- 1-yl)pyridazine-3- carbonyl)piperidin-4- yl)butyl)-3-(pyridin-3- yl)acrylamide | (E)-N-(4-(1-(6-(4-(4- ((2-(2,6- dioxopiperidin-3-yl)- 1-oxoisoindolin-4- yl)thio)butyl)piperazin- 1-yl)pyridazine-3- carbonyl)piperidin-4- yl)butyl)-3-(pyridin-3- yl)acrylamide |
| SIAI S632 039 | 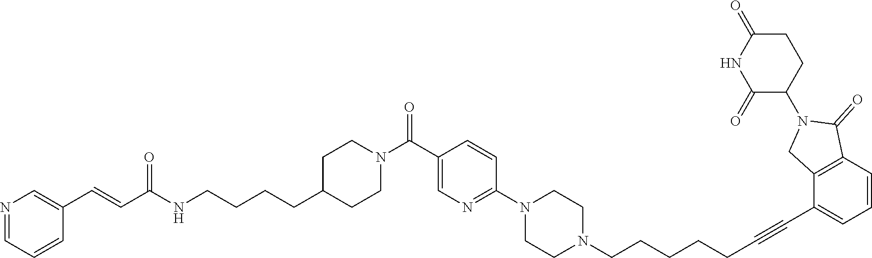 [SMILES: O=C(/C=C/c1cccnc1)NCCCCC1CCN(C(=O)c2ccc(N3CCN(CCCCCC#Cc4cccc5c4CN(C4CCC(=O)NC4=O)C5=O)CC3)nc2)CC1] | (E)-N-(4-(1-(6-(4-(7-(2- (2,6-dioxopiperidin-3- yl)-1-oxoisoindolin-4- yl)hept-6-yn-1- yl)piperazin-1- yl)nicotinoyl)piperidin- 4-yl)butyl)-3-(pyridin-3- yl)acrylamide | (E)-N-(4-(1-(6-(4-(7- (2-(2,6-dioxopiperidin- 3-yl)-1-oxoisoindolin- 4-yl)hept-6-yn-1- yl)piperazin-1- yl)nicotinoyl)piperidin- 4-yl)butyl)-3-(pyridin- 3-yl)acrylamide |
| SIAI S632 040 | 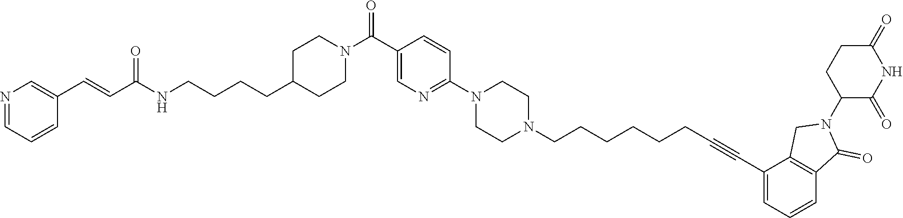 [SMILES: O=C(/C=C/c1cccnc1)NCCCCC1CCN(C(=O)c2ccc(N3CCN(CCCCCCC#Cc4cccc5c4CN(C4CCC(=O)NC4=O)C5=O)CC3)nc2)CC1] | (E)-N-(4-(1-(6-(4-(8-(2- (2,6-dioxopiperidin-3- yl)-1-oxoisoindolin-4- yl)oct-7-yn-1- yl)piperazin-1- yl)nicotinoyl)piperidin- 4-yl)butyl)-3-(pyridin-3- yl)acrylamide | (E)-N-(4-(1-(6-(4-(8- (2-(2,6-dioxopiperidin- 3-yl)-1-oxoisoindolin- 4-yl)oct-7-yn-1- yl)piperazin-1- yl)nicotinoyl)piperidin- 4-yl)butyl)-3-(pyridin- 3-yl)acrylamide |
| SIAI S632 041 | 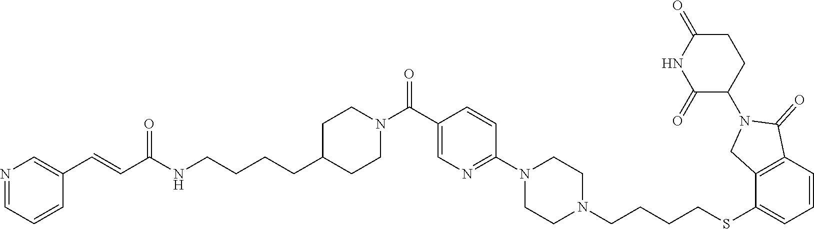 [SMILES: O=C(/C=C/c1cccnc1)NCCCCC1CCN(C(=O)c2ccc(N3CCN(CCCCSc4cccc5c4CN(C4CCC(=O)NC4=O)C5=O)CC3)nc2)CC1] | (E)-N-(4-(1-(6-(4-(4- ((2-(2,6-dioxopiperidin- 3-yl)-1-oxoisoindolin-4- yl)thio)butyl) piperazin-1- yl)nicotinoyl)piperidin- 4-yl)butyl)-3-(pyridin- 3-yl)acrylamide | (E)-N-(4-(1-(6-(4-(4- ((2-(2,6- dioxopiperidin-3-yl)- 1-oxoisoindolin-4- yl)thio)butyl) piperazin-1- yl)nicotinoyl) piperidin-4-yl) butyl)-3-(pyridin- 3-yl)acrylamide |
| SIAI S631 079 | 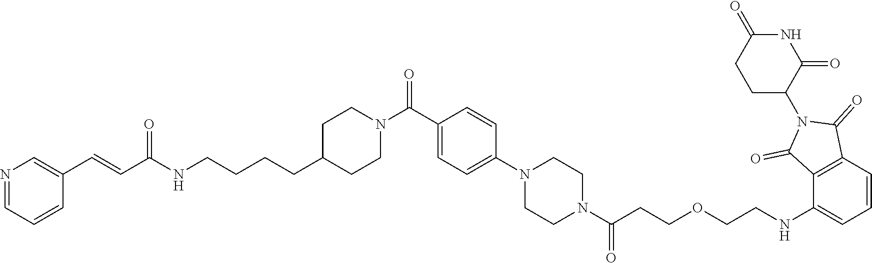 [SMILES: O=C(/C=C/c1cccnc1)NCCCCC1CCN(C(=O)c2ccc(N3CCN(C(=O)CCOCCNc4cccc5c4C(=O)N(C4CCC(=O)NC4=O)C5=O)CC3)cc2)CC1] | (E)-N-(4-(1-(4-(4-(3-(2- ((2-(2,6-dioxopiperidin- 3-yl)-1,3- dioxoisoindolin-4- yl)amino)ethoxy)propan oyl)piperazin-1- yl)benzoyl)piperidin-4- yl)butyl)-3-(pyridin-3- yl)acrylamide | (E)-N-(4-(1-(4-(4-(3- (2-((2-(2,6- dioxopiperidin-3-yl)- 1,3-dioxoisoindolin-4- yl)amino)ethoxy) propanoyl)piperazin- 1-yl)benzoyl) piperidin-4- yl)butyl)-3-(pyridin- 3-yl)acrylamide |
| SIAI S631 109 | 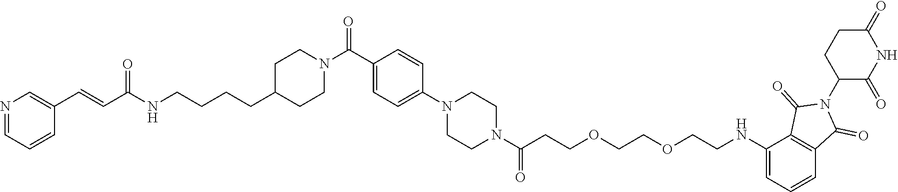 [SMILES: O=C(/C=C/c1cccnc1)NCCCCC1CCN(C(=O)c2ccc(N3CCN(C(=O)CCOCCOCCNc4cccc5c4C(=O)N(C4CCC(=O)NC4=O)C5=O)CC3)cc2)CC1] | (E)-N-(4-(1-(4-(4-(3-(2- (2-((2-(2,6- dioxopiperidin-3-yl)- 1,3-dioxoisoindolin-4- yl)amino)ethoxy)ethoxy )propanoyl)piperazin-1- yl)benzoyl)piperidin-4- yl)butyl)-3-(pyridin-3- yl)acrylamide | (E)-N-(4-(1-(4-(4-(3- (2-(2-((2-(2,6- dioxopiperidin-3-yl)- 1,3-dioxoisoindolin-4- yl)amino)ethoxy) ethoxy) propanoyl)piperazin- 1-yl)benzoyl)piperidin- 4-yl)butyl)-3-(pyridin- 3-yl)acrylamide |
| SIAI S631 119 | 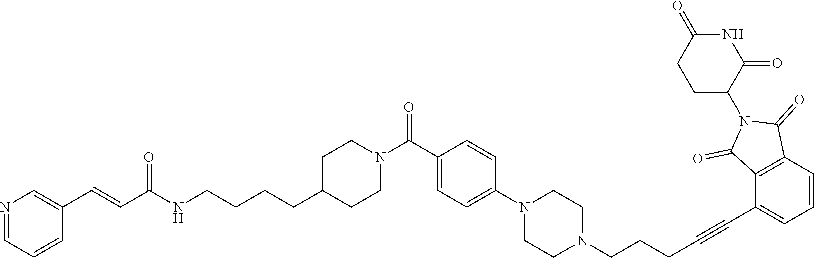 [SMILES: O=C(/C=C/c1cccnc1)NCCCCC1CCN(C(=O)c2ccc(N3CCN(CCCC#Cc4cccc5c4C(=O)N(C4CCC(=O)NC4=O)C5=O)CC3)cc2)CC1] | (E)-N-(4-(1-(4-(4-(5-(2- (2,6-dioxopiperidin-3- yl)-1,3-dioxoisoindolin- 4-yl)pent-4-yn-1- yl)piperazin-1- yl)benzoyl)piperidin-4- yl)butyl)-3-(pyridin-3- yl)acrylamide | (E)-N-(4-(1-(4-(4-(5- (2-(2,6-dioxopiperidin- 3-yl)-1,3- dioxoisoindolin-4- yl)pent-4-yn-1- yl)piperazin-1- yl)benzoyl)piperidin-4- yl)butyl)-3-(pyridin-3- yl)acrylamide |
| SIAI S631 120 | 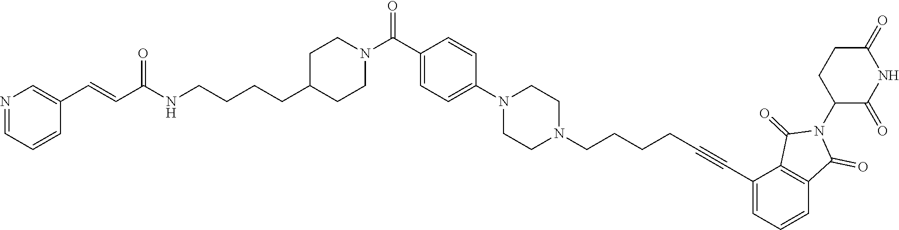 [SMILES: O=C(/C=C/c1cccnc1)NCCCCC1CCN(C(=O)c2ccc(N3CCN(CCCCC#Cc4cccc5c4C(=O)N(C4CCC(=O)NC4=O)C5=O)CC3)cc2)CC1] | (E)-N-(4-(1-(4-(4-(6-(2- (2,6-dioxopiperidin-3- yl)-1,3-dioxoisoindolin- 4-yl)hex-5-yn-1- yl)piperazin-1- yl)benzoyl)piperidin-4- yl)butyl)-3-(pyridin-3- yl)acrylamide | (E)-N-(4-(1-(4-(4-(6- (2-(2,6-dioxopiperidin- 3-yl)-1,3- dioxoisoindolin-4- yl)hex-5-yn-1- yl)piperazin-1- yl)benzoyl)piperidin-4- yl)butyl)-3-(pyridin-3- yl)acrylamide |
| SIAI S631 108 | 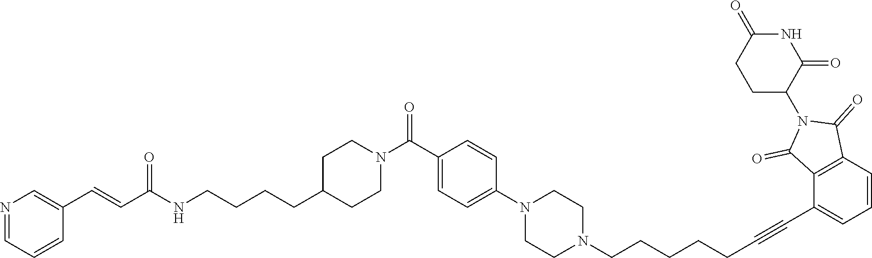 [SMILES: O=C(/C=C/c1cccnc1)NCCCCC1CCN(C(=O)c2ccc(N3CCN(CCCCCC#Cc4cccc5c4C(=O)N(C4CCC(=O)NC4=O)C5=O)CC3)cc2)CC1] | (E)-N-(4-(1-(4-(4-(7-(2- (2,6-dioxopiperidin-3- yl)-1,3-dioxoisoindolin- 4-yl)hept-6-yn-1- yl)piperazin-1- yl)benzoyl)piperidin-4- yl)butyl)-3-(pyridin-3- yl)acrylamide | (E)-N-(4-(1-(4-(4-(7- (2-(2,6-dioxopiperidin- 3-yl)-1,3- dioxoisoindolin-4- yl)hept-6-yn-1- yl)piperazin-1- yl)benzoyl)piperidin-4- yl)butyl)-3-(pyridin-3- yl)acrylamide |
| SIAI S631 121 | 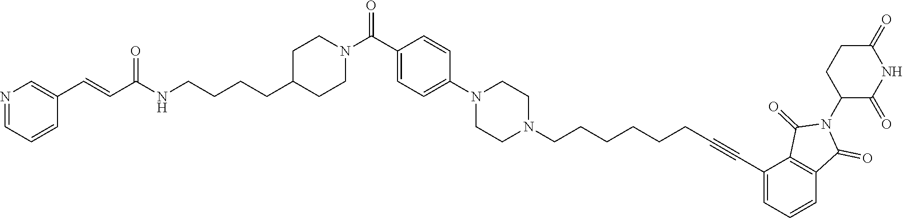 [SMILES: O=C(/C=C/c1cccnc1)NCCCCC1CCN(C(=O)c2ccc(N3CCN(CCCCCCC#Cc4cccc5c4C(=O)N(C4CCC(=O)NC4=O)C5=O)CC3)cc2)CC1] | (E)-N-(4-(1-(4-(4-(8-(2- (2,6-dioxopiperidin-3- yl)-1,3-dioxoisoindolin- 4-yl)oct-7-yn-1- yl)piperazin-1- yl)benzoyl)piperidin-4- yl)butyl)-3-(pyridin-3- yl)acrylamide | (E)-N-(4-(1-(4-(4-(8- (2-(2,6-dioxopiperidin- 3-yl)-1,3- dioxoisoindolin-4- yl)oct-7-yn-1- yl)piperazin-1- yl)benzoyl)piperidin-4- yl)butyl)-3-(pyridin-3- yl)acrylamide |
| SIAI S631 111 | 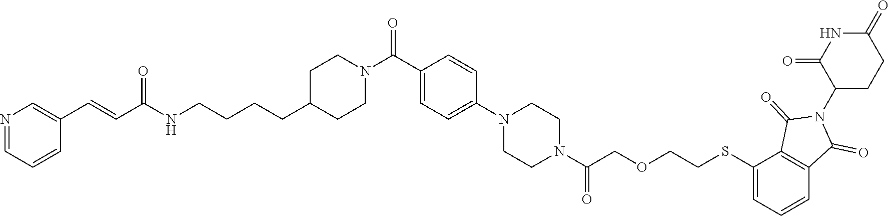 [SMILES: O=C(/C=C/c1cccnc1)NCCCCC1CCN(C(=O)c2ccc(N3CCN(C(=O)COCCSc4cccc5c4C(=O)N(C4CCC(=O)NC4=O)C5=O)CC3)cc2)CC1] | (E)-N-(4-(1-(4-(4-(2-(2- ((2-(2,6-dioxopiperidin- 3-yl)-1,3- dioxoisoindolin-4- yl)thio)ethoxy)acetyl) piperazin-1- yl)benzoyl)piperidin-4- yl)butyl)-3-(pyridin-3- yl)acrylamide | (E)-N-(4-(1-(4-(4-(2- (2-((2-(2,6- dioxopiperidin-3-yl)- 1,3-dioxoisoindolin-4- yl)thio)ethoxy)acetyl) piperazin-1- yl)benzoyl)piperidin-4- yl)butyl)-3-(pyridin-3- yl)acrylamide |
| | 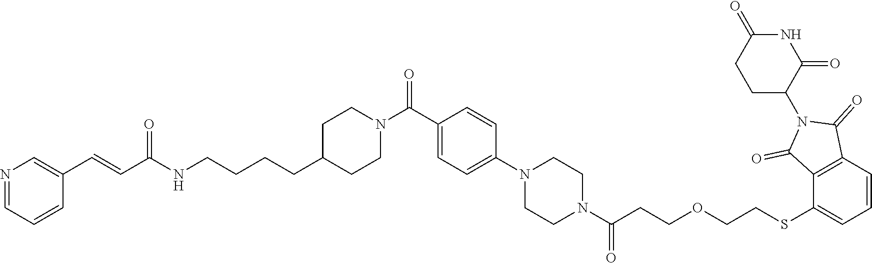 [SMILES: O=C(/C=C/c1cccnc1)NCCCCC1CCN(C(=O)c2ccc(N3CCN(C(=O)CCOCCSc4cccc5c4C(=O)N(C4CCC(=O)NC4=O)C5=O)CC3)cc2)CC1] | (E)-N-(4-(1-(4-(4-(3-(2- ((2-(2,6-dioxopiperidin- 3-yl)-1,3- dioxoisoindolin-4- yl)thio)ethoxy) propanoyl)piperazin-1- yl)benzoyl)piperidin-4- yl)butyl)-3-(pyridin-3- yl)acrylamide | (E)-N-(4-(1-(4-(4-(3- (2-((2-(2,6- dioxopiperidin-3-yl)- 1,3-dioxoisoindolin-4- yl)thio)ethoxy) propanoyl)piperazin- 1-yl)benzoyl) piperidin-4- yl)butyl)-3-(pyridin- 3-yl)acrylamide |
| SIAI S631 112 | 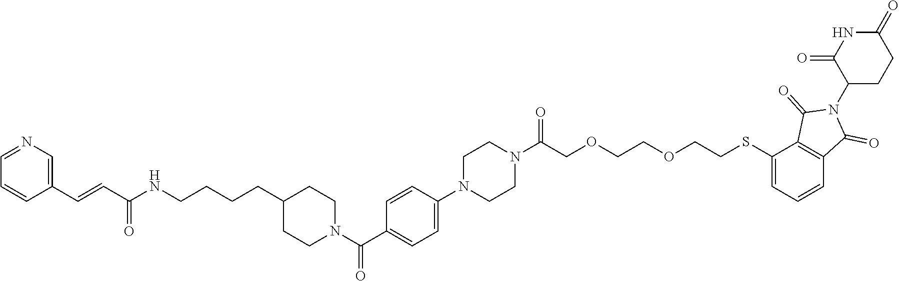 [SMILES: O=C(/C=C/c1cccnc1)NCCCCC1CCN(C(=O)c2ccc(N3CCN(C(=O)COCCOCCSc4cccc5c4C(=O)N(C4CCC(=O)NC4=O)C5=O)CC3)cc2)CC1] | (E)-N-(4-(1-(4-(4-(2-(2- (2-((2-(2,6- dioxopiperidin-3-yl)- 1,3-dioxoisoindolin-4- yl)thio)ethoxy)ethoxy) acetyl)piperazin-1- yl)benzoyl)piperidin-4- yl)butyl)-3-(pyridin-3- yl)acrylamide | (E)-N-(4-(1-(4-(4-(2- (2-(2-((2-(2,6- dioxopiperidin-3-yl)- 1,3-dioxoisoindolin-4- yl)thio)ethoxy)ethoxy) acetyl)piperazin-1- yl)benzoyl)piperidin-4- yl)butyl)-3-(pyridin-3- yl)acrylamide |
| | 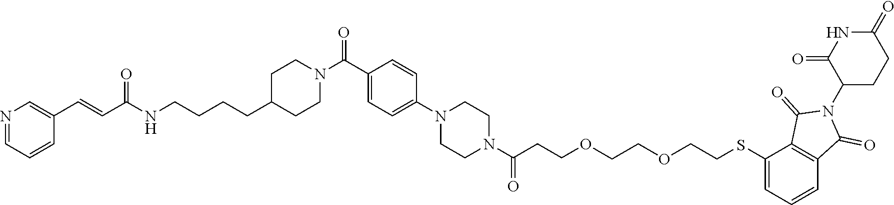 [SMILES: O=C(/C=C/c1cccnc1)NCCCCC1CCN(C(=O)c2ccc(N3CCN(C(=O)CCOCCOCCSc4cccc5c4C(=O)N(C4CCC(=O)NC4=O)C5=O)CC3)cc2)CC1] | (E)-N-(4-(1-(4-(4-(3-(2- (2-((2-(2,6- dioxopiperidin-3-yl)- 1,3-dioxoisoindolin-4- yl)thio)ethoxy)ethoxy) propanoyl)piperazin-1- yl)benzoyl)piperidin-4- yl)butyl)-3-(pyridin-3- yl)acrylamide | (E)-N-(4-(1-(4-(4-(3- (2-(2-((2-(2,6- dioxopiperidin-3-yl)- 1,3-dioxoisoindolin-4- yl)thio)ethoxy)ethoxy) propanoyl)piperazin-1- yl)benzoyl)piperidin-4- yl)butyl)-3-(pyridin-3- yl)acrylamide |
| SIAI S631 113 | 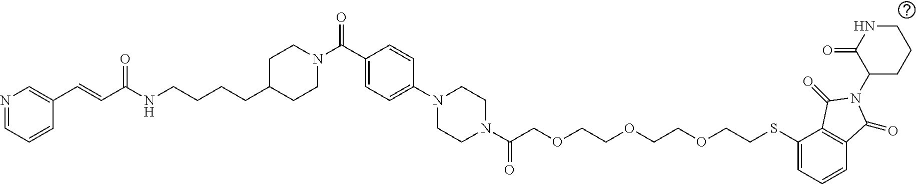 [SMILES: O=C(/C=C/c1cccnc1)NCCCCC1CCN(C(=O)c2ccc(N3CCN(C(=O)COCCOCCOCCSc4cccc5c4C(=O)N(C4CCCNC4=O)C5=O)CC3)cc2)CC1] | (E)-N-(4-(1-(4-(4-(2-(2- (2-(2-((2-(2,6- dioxopiperidin-3-yl)- 1,3-dioxoisoindolin-4- yl)thio)ethoxy)ethoxy) ethoxy)acetyl)piperazin- 1-yl)benzoyl)piperidin- 4-yl)butyl)-3-(pyridin- 3-yl)acrylamide | (E)-N-(4-(1-(4-(4-(2- (2-(2-(2-((2-(2,6- dioxopiperidin-3-yl)- 1,3-dioxoisoindolin-4- yl)thio)ethoxy)ethoxy) ethoxy)acetyl) piperazin-1-yl) benzoyl)piperidin- 4-yl)butyl)-3-(pyridin- 3-yl)acrylamide |
| | 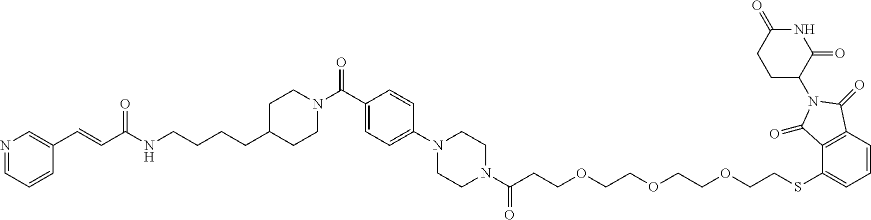 [SMILES: O=C(/C=C/c1cccnc1)NCCCCC1CCN(C(=O)c2ccc(N3CCN(C(=O)CCOCCOCCOCCSc4cccc5c4C(=O)N(C4CCC(=O)NC4=O)C5=O)CC3)cc2)CC1] | (E)-N-(4-(1-(4-(4-(3-(2- (2-(2-((2-(2,6- dioxopiperidin-3-yl)- 1,3-dioxoisoindolin-4- yl)thio)ethoxy)ethoxy) ethoxy)propanoyl) piperazin-1- yl)benzoyl)piperidin-4- yl)butyl)-3-(pyridin-3- yl)acrylamide | (E)-N-(4-(1-(4-(4-(3- (2-(2-(2-((2-(2,6- dioxopiperidin-3-yl)- 1,3-dioxoisoindolin-4- yl)thio)ethoxy)ethoxy) ethoxy)propanoyl) piperazin-1- yl)benzoyl)piperidin-4- yl)butyl)-3-(pyridin-3- yl)acrylamide |
| SIAI S631 114 | 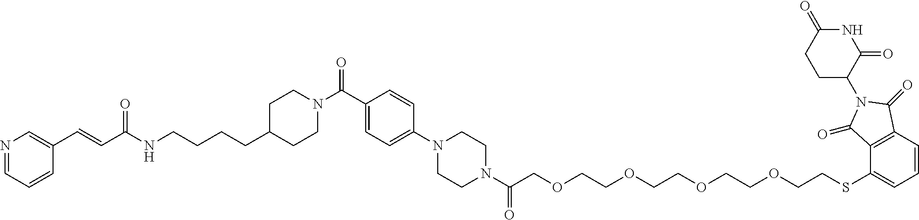 [SMILES: O=C(/C=C/c1cccnc1)NCCCCC1CCN(C(=O)c2ccc(N3CCN(C(=O)COCCOCCOCCOCCSc4cccc5c4C(=O)N(C4CCC(=O)NC4=O)C5=O)CC3)cc2)CC1] | (E)-N-(4-(1-(4-(4-(14- ((2-(2,6-dioxopiperidin- 3-yl)-1,3- dioxoisoindolin-4- yl)thio)-3,6,9,12- tetraoxatetradecanoyl) piperazin-1- yl)benzoyl)piperidin-4- yl)butyl)-3-(pyridin-3- yl)acrylamide | (E)-N-(4-(1-(4-(4-(14- ((2-(2,6- dioxopiperidin-3-yl)- 1,3-dioxoisoindolin-4- yl)thio)-3,6,9,12- tetraoxatetradecanoyl) piperazin-1- yl)benzoyl)piperidin-4- yl)butyl)-3-(pyridin-3- yl)acrylamide |
| SIAI S631 115 | 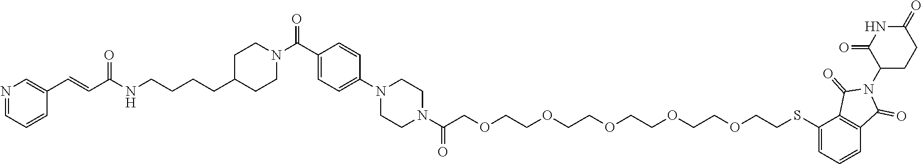 [SMILES: O=C(/C=C/c1cccnc1)NCCCCC1CCN(C(=O)c2ccc(N3CCN(C(=O)COCCOCCOCCOCCOCCSc4cccc5c4C(=O)N(C4CCC(=O)NC4=O)C5=O)CC3)cc2)CC1] | (E)-N-(4-(1-(4-(4-(17- ((2-(2,6-dioxopiperidin- 3-yl)-1,3- dioxoisoindolin-4- yl)thio)-3,6,9,12,15- pentaoxaheptadecanoyl) piperazin-1- yl)benzoyl)piperidin-4- yl)butyl)-3-(pyridin-3- yl)acrylamide | (E)-N-(4-(1-(4-(4-(17- ((2-(2,6- dioxopiperidin-3-yl)- 1,3-dioxoisoindolin-4- yl)thio)-3,6,9,12,15- pentaoxaheptadecanoyl) piperazin-1- yl)benzoyl)piperidin-4- yl)butyl)-3-(pyridin-3- yl)acrylamide |
| SIAI S631 118 | 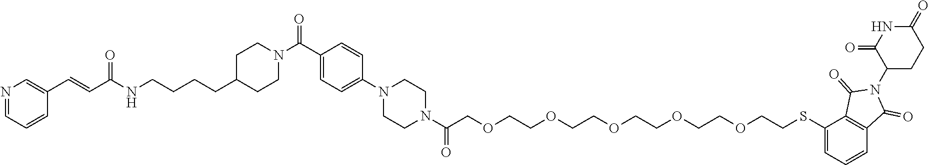 [SMILES: O=C(/C=C/c1cccnc1)NCCCCC1CCN(C(=O)c2ccc(N3CCN(C(=O)COCCOCCOCCOCCOCCSc4cccc5c4C(=O)N(C4CCC(=O)NC4=O)C5=O)CC3)cc2)CC1] | (E)-N-(4-(1-(4-(4-(17- ((2-(2,6-dioxopiperidin- 3-yl)-1-oxoisoindolin-4- yl)thio)-3,6,9,12,15- pentaoxaheptadecanoyl) piperazin-1- yl)benzoyl)piperidin-4- yl)butyl)-3-(pyridin-3- yl)acrylamide | (E)-N-(4-(1-(4-(4-(17- ((2-(2,6- dioxopiperidin-3-yl)-1- oxoisoindolin-4- yl)thio)-3,6,9,12,15- pentaoxaheptadecanoyl) piperazin-1- yl)benzoyl)piperidin-4- yl)butyl)-3-(pyridin-3- yl)acrylamide |
| | 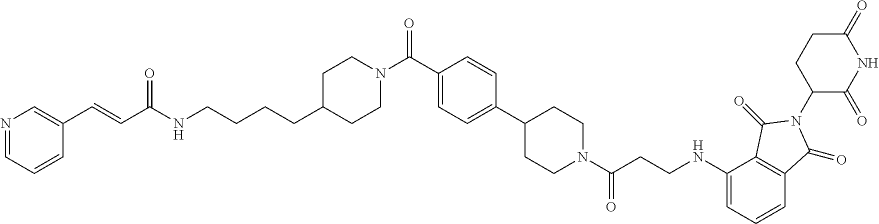 [SMILES: O=C(/C=C/c1cccnc1)NCCCCC1CCN(C(=O)c2ccc(C3CCN(C(=O)CCNc4cccc5c4C(=O)N(C4CCC(=O)NC4=O)C5=O)CC3)cc2)CC1] | (E)-N-(4-(1-(4-(1-(3- ((2-(2,6-dioxopiperidin- 3-yl)-1,3- dioxoisoindolin-4- yl)amino)propanoyl) piperidin-4- yl)benzoyl)piperidin-4- yl)butyl)-3-(pyridin-3- yl)acrylamide | (E)-N-(4-(1-(4-(1-(3- ((2-(2,6- dioxopiperidin-3-yl)- 1,3-dioxoisoindolin-4- yl)amino)propanoyl) piperidin-4- yl)benzoyl)piperidin-4- yl)butyl)-3-(pyridin-3- yl)acrylamide |
| | 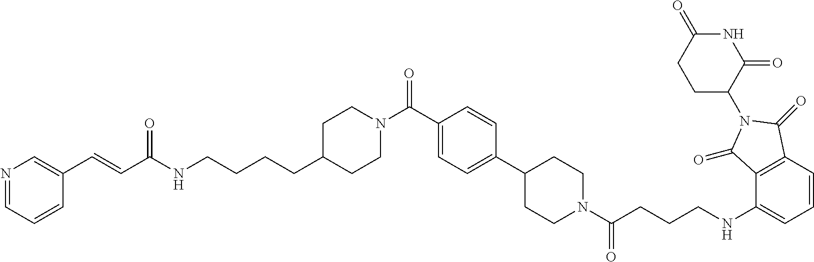 [SMILES: O=C(/C=C/c1cccnc1)NCCCCC1CCN(C(=O)c2ccc(C3CCN(C(=O)CCCNc4cccc5c4C(=O)N(C4CCC(=O)NC4=O)C5=O)CC3)cc2)CC1] | (E)-N-(4-(1-(4-(1-(4- ((2-(2,6-dioxopiperidin- 3-yl)-1,3- dioxoisoindolin-4- yl)amino)butanoyl) piperidin-4- yl)benzoyl)piperidin-4- yl)butyl)-3-(pyridin-3- yl)acrylamide | (E)-N-(4-(1-(4-(1-(4- ((2-(2,6- dioxopiperidin-3-yl)- 1,3-dioxoisoindolin-4- yl)amino)butanoyl) piperidin-4- yl)benzoyl)piperidin-4- yl)butyl)-3-(pyridin-3- yl)acrylamide |
| | 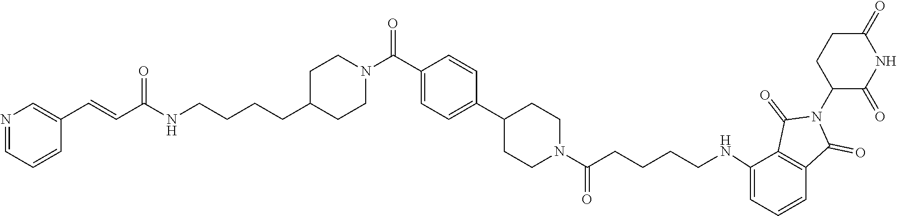 [SMILES: O=C(/C=C/c1cccnc1)NCCCCC1CCN(C(=O)c2ccc(C3CCN(C(=O)CCCCNc4cccc5c4C(=O)N(C4CCC(=O)NC4=O)C5=O)CC3)cc2)CC1] | (E)-N-(4-(1-(4-(1-(5- ((2-(2,6-dioxopiperidin- 3-yl)-1,3- dioxoisoindolin-4- yl)amino)pentanoyl) piperidin-4- yl)benzoyl)piperidin-4- yl)butyl)-3-(pyridin-3- yl)acrylamide | (E)-N-(4-(1-(4-(1-(5- ((2-(2,6- dioxopiperidin-3-yl)- 1,3-dioxoisoindolin-4- yl)amino)pentanoyl) piperidin-4- yl)benzoyl)piperidin-4- yl)butyl)-3-(pyridin-3- yl)acrylamide |
| | 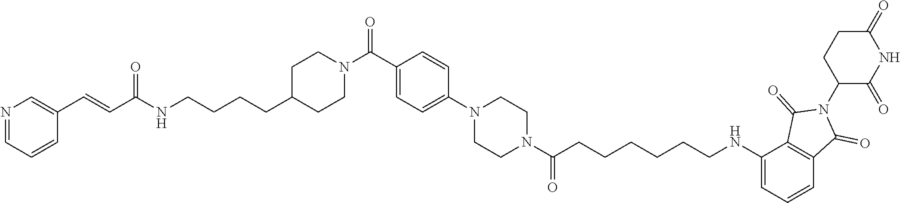 [SMILES: O=C(/C=C/c1cccnc1)NCCCCC1CCN(C(=O)c2ccc(N3CCN(C(=O)CCCCCCNc4cccc5c4C(=O)N(C4CCC(=O)NC4=O)C5=O)CC3)cc2)CC1] | (E)-N-(4-(1-(4-(1-(7- ((2-(2,6-dioxopiperidin- 3-yl)-1,3- dioxoisoindolin-4- yl)amino)heptanoyl) piperidin-4- yl)benzoyl)piperidin-4- yl)butyl)-3-(pyridin-3- yl)acrylamide | (E)-N-(4-(1-(4-(1-(7- ((2-(2,6- dioxopiperidin-3-yl)- 1,3-dioxoisoindolin-4- yl)amino)heptanoyl) piperidin-4- yl)benzoyl)piperidin-4- yl)butyl)-3-(pyridin-3- yl)acrylamide |
| | 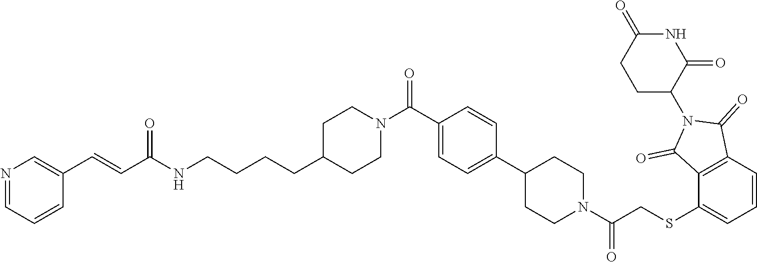 [SMILES: O=C(/C=C/c1cccnc1)NCCCCC1CCN(C(=O)c2ccc(C3CCN(C(=O)CSc4cccc5c4C(=O)N(C4CCC(=O)NC4=O)C5=O)CC3)cc2)CC1] | (E)-N-(4-(1-(4-(1-(2- ((2-(2,6-dioxopiperidin- 3-yl)-1,3- dioxoisoindolin-4- yl)thio)acetyl)piperidin- 4-yl)benzoyl)piperidin- 4-yl)butyl)-3-(pyridin-3- yl)acrylamide | (E)-N-(4-(1-(4-(1-(2- ((2-(2,6- dioxopiperidin-3-yl)- 1,3-dioxoisoindolin-4- yl)thio)acetyl) piperidin-4- yl)benzoyl)piperidin-4- yl)butyl)-3-(pyridin-3- yl)acrylamide |
| | 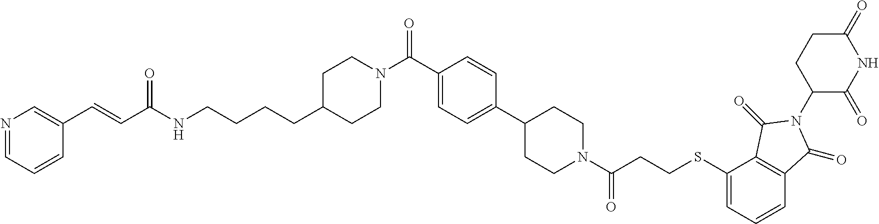 [SMILES: O=C(/C=C/c1cccnc1)NCCCCC1CCN(C(=O)c2ccc(C3CCN(C(=O)CCSc4cccc5c4C(=O)N(C4CCC(=O)NC4=O)C5=O)CC3)cc2)CC1] | (E)-N-(4-(1-(4-(1-(3- ((2-(2,6-dioxopiperidin- 3-yl)-1,3- dioxoisoindolin-4- yl)thio)propanoyl) piperidin-4- yl)benzoyl)piperidin-4- yl)butyl)-3-(pyridin-3- yl)acrylamide | (E)-N-(4-(1-(4-(1-(3- ((2-(2,6- dioxopiperidin-3-yl)- 1,3-dioxoisoindolin-4- yl)thio)propanoyl) piperidin-4- yl)benzoyl)piperidin-4- yl)butyl)-3-(pyridin-3- yl)acrylamide |
| | 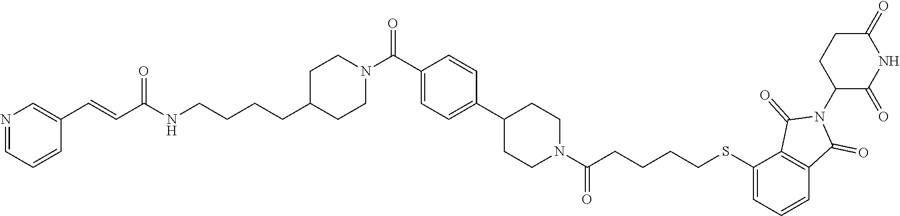 [SMILES: O=C(/C=C/c1cccnc1)NCCCCC1CCN(C(=O)c2ccc(C3CCN(C(=O)CCCCSc4cccc5c4C(=O)N(C4CCC(=O)NC4=O)C5=O)CC3)cc2)CC1] | (E)-N-(4-(1-(4-(1-(5- ((2-(2,6-dioxopiperidin- 3-yl)-1,3- dioxoisoindolin-4- yl)thio)pentanoyl) piperidin-4- yl)benzoyl)piperidin-4- yl)butyl)-3-(pyridin-3- yl)acrylamide | (E)-N-(4-(1-(4-(1-(5- ((2-(2,6- dioxopiperidin-3-yl)- 1,3-dioxoisoindolin-4- yl)thio)pentanoyl) piperidin-4- yl)benzoyl)piperidin-4- yl)butyl)-3-(pyridin-3- yl)acrylamide |
| | 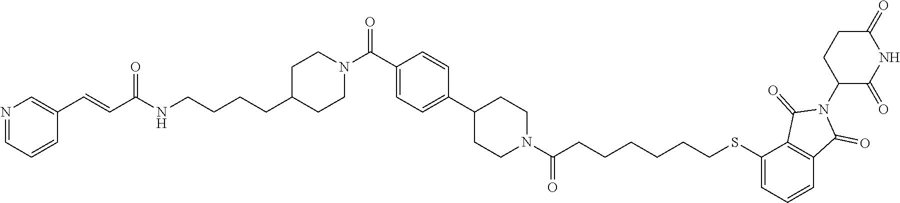 [SMILES: O=C(/C=C/c1cccnc1)NCCCCC1CCN(C(=O)c2ccc(C3CCN(C(=O)CCCCCCSc4cccc5c4C(=O)N(C4CCC(=O)NC4=O)C5=O)CC3)cc2)CC1] | (E)-N-(4-(1-(4-(1-(7- ((2-(2,6-dioxopiperidin- 3-yl)-1,3- dioxoisoindolin-4- yl)thio)heptanoyl) piperidin-4- yl)benzoyl)piperidin-4- yl)butyl)-3-(pyridin-3- yl)acrylamide | (E)-N-(4-(1-(4-(1-(7- ((2-(2,6- dioxopiperidin-3-yl)- 1,3-dioxoisoindolin-4- yl)thio)heptanoyl) piperidin-4- yl)benzoyl)piperidin-4- yl)butyl)-3-(pyridin-3- yl)acrylamide |
| | 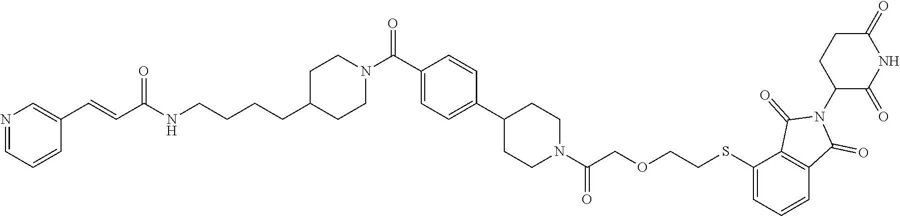 [SMILES: O=C(/C=C/c1cccnc1)NCCCCC1CCN(C(=O)c2ccc(C3CCN(C(=O)COCCSc4cccc5c4C(=O)N(C4CCC(=O)NC4=O)C5=O)CC3)cc2)CC1] | (E)-N-(4-(1-(4-(1-(2-(2- ((2-(2,6-dioxopiperidin- 3-yl)-1,3- dioxoisoindolin-4- yl)thio)ethoxy)acetyl) piperidin-4- yl)benzoyl)piperidin-4- yl)butyl)-3-(pyridin-3- yl)acrylamide | (E)-N-(4-(1-(4-(1-(2- (2-((2-(2,6- dioxopiperidin-3-yl)- 1,3-dioxoisoindolin-4- yl)thio)ethoxy)acetyl) piperidin-4- yl)benzoyl)piperidin-4- yl)butyl)-3-(pyridin-3- yl)acrylamide |
| | 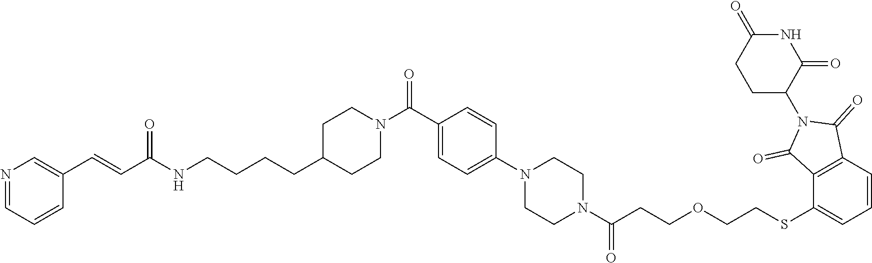 [SMILES: O=C(/C=C/c1cccnc1)NCCCCC1CCN(C(=O)c2ccc(N3CCN(C(=O)CCOCCSc4cccc5c4C(=O)N(C4CCC(=O)NC4=O)C5=O)CC3)cc2)CC1] | (E)-N-(4-(1-(4-(1-(3-(2- ((2-(2,6-dioxopiperidin- 3-yl)-1,3- dioxoisoindolin-4- yl)thio)ethoxy) propanoyl)piperidin-4- yl)benzoyl)piperidin-4- yl)butyl)-3-(pyridin-3- yl)acrylamide | (E)-N-(4-(1-(4-(1-(3- (2-((2-(2,6- dioxopiperidin-3-yl)- 1,3-dioxoisoindolin-4- yl)thio)ethoxy) propanoyl)piperidin-4- yl)benzoyl)piperidin-4- yl)butyl)-3-(pyridin-3- yl)acrylamide |
| | 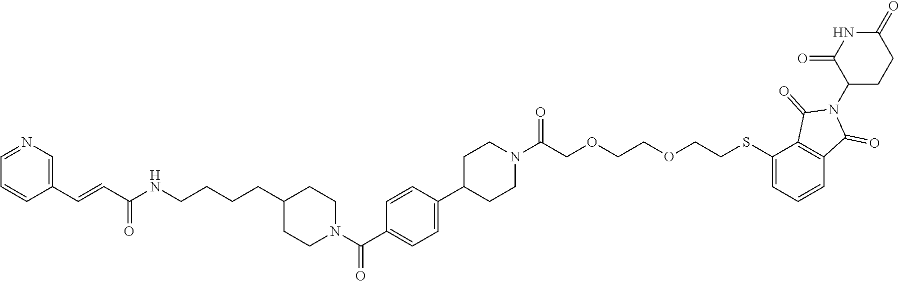 [SMILES: O=C(/C=C/c1cccnc1)NCCCCC1CCN(C(=O)c2ccc(C3CCN(C(=O)COCCOCCSc4cccc5c4C(=O)N(C4CCC(=O)NC4=O)C5=O)CC3)cc2)CC1] | (E)-N-(4-(1-(4-(1-(2-(2- (2-((2-(2,6- dioxopiperidin-3-yl)- 1,3-dioxoisoindolin-4- yl)thio)ethoxy)ethoxy) acetyl)piperidin-4- yl)benzoyl)piperidin-4- yl)butyl)-3-(pyridin-3- yl)acrylamide | (E)-N-(4-(1-(4-(1-(2- (2-(2-((2-(2,6- dioxopiperidin-3-yl)- 1,3-dioxoisoindolin-4- yl)thio)ethoxy)ethoxy) acetyl)piperidin-4- yl)benzoyl)piperidin-4- yl)butyl)-3-(pyridin-3- yl)acrylamide |
| | 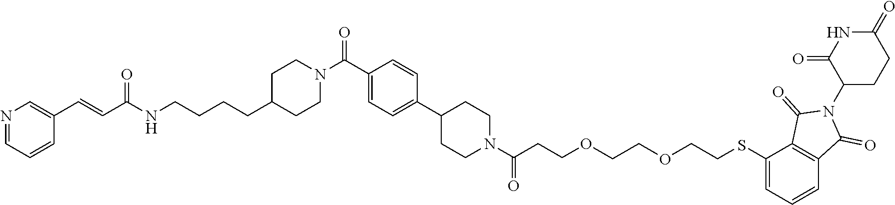 [SMILES: O=C(/C=C/c1cccnc1)NCCCCC1CCN(C(=O)c2ccc(C3CCN(C(=O)CCOCCOCCSc4cccc5c4C(=O)N(C4CCC(=O)NC4=O)C5=O)CC3)cc2)CC1] | (E)-N-(4-(1-(4-(1-(3-(2- (2-((2-(2,6- dioxopiperidin-3-yl)- 1,3-dioxoisoindolin-4- yl)thio)ethoxy)ethoxy) propanoyl)piperidin-4- yl)benzoyl)piperidin-4- yl)butyl)-3-(pyridin-3- yl)acrylamide | (E)-N-(4-(1-(4-(1-(3- (2-(2-((2-(2,6- dioxopiperidin-3-yl)- 1,3-dioxoisoindolin-4- yl)thio)ethoxy)ethoxy) propanoyl)piperidin-4- yl)benzoyl)piperidin-4- yl)butyl)-3-(pyridin-3- yl)acrylamide |
| | 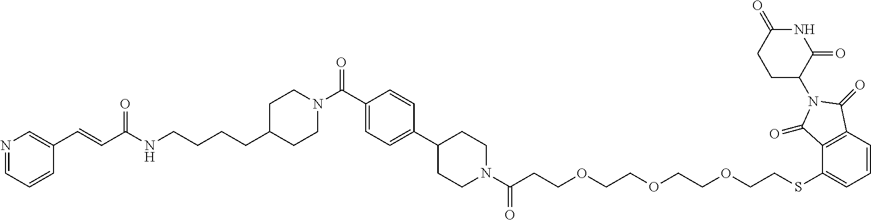 [SMILES: O=C(/C=C/c1cccnc1)NCCCCC1CCN(C(=O)c2ccc(C3CCN(C(=O)CCOCCOCCOCCSc4cccc5c4C(=O)N(C4CCC(=O)NC4=O)C5=O)CC3)cc2)CC1] | (E)-N-(4-(1-(4-(1-(3-(2- (2-(2-((2-(2,6- dioxopiperidin-3-yl)- 1,3-dioxoisoindolin-4- yl)thio)ethoxy)ethoxy) ethoxy)propanoyl) piperidin-4- yl)benzoyl)piperidin-4- yl)butyl)-3-(pyridin-3- yl)acrylamide | (E)-N-(4-(1-(4-(1-(3- (2-(2-(2-((2-(2,6- dioxopiperidin-3-yl)- 1,3-dioxoisoindolin-4- yl)thio)ethoxy)ethoxy) ethoxy)propanoyl) piperidin-4- yl)benzoyl)piperidin-4- yl)butyl)-3-(pyridin-3- yl)acrylamide |
| | 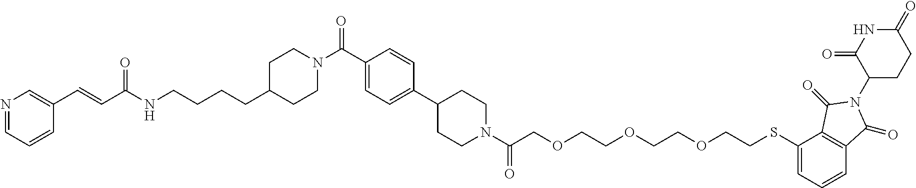 [SMILES: O=C(/C=C/c1cccnc1)NCCCCC1CCN(C(=O)c2ccc(C3CCN(C(=O)COCCOCCOCCSc4cccc5c4C(=O)N(C4CCC(=O)NC4=O)C5=O)CC3)cc2)CC1] | (E)-N-(4-(1-(4-(1-(2-(2- (2-(2-((2-(2,6- dioxopiperidin-3-yl)- 1,3-dioxoisoindolin-4- yl)thio)ethoxy)ethoxy) ethoxy)acetyl) piperidin-4- yl)benzoyl)piperidin-4- yl)butyl)-3-(pyridin-3- yl)acrylamide | (E)-N-(4-(1-(4-(1-(2- (2-(2-(2-((2-(2,6- dioxopiperidin-3-yl)- 1,3-dioxoisoindolin-4- yl)thio)ethoxy)ethoxy) ethoxy)acetyl)piperidin- 4-yl)benzoyl)piperidin- 4-yl)butyl)-3-(pyridin- 3-yl)acrylamide |
| | 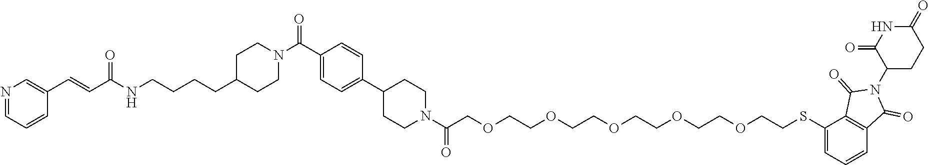 [SMILES: O=C(/C=C/c1cccnc1)NCCCCC1CCN(C(=O)c2ccc(C3CCN(C(=O)COCCOCCOCCOCCOCCSc4cccc5c4C(=O)N(C4CCC(=O)NC4=O)C5=O)CC3)cc2)CC1] | (E)-N-(4-(1-(4-(1-(17- ((2-(2,6-dioxopiperidin- 3-yl)-1,3- dioxoisoindolin-4- yl)thio)-3,6,9,12,15- pentaoxaheptadecanoyl) piperidin-4- yl)benzoyl)piperidin-4- yl)butyl)-3-(pyridin-3- yl)acrylamide | (E)-N-(4-(1-(4-(1-(17- ((2-(2,6- dioxopiperidin-3-yl)- 1,3-dioxoisoindolin-4- yl)thio)-3,6,9,12,15- pentaoxaheptadecanoyl) piperidin-4- yl)benzoyl)piperidin-4- yl)butyl)-3-(pyridin-3- yl)acrylamide |
| | 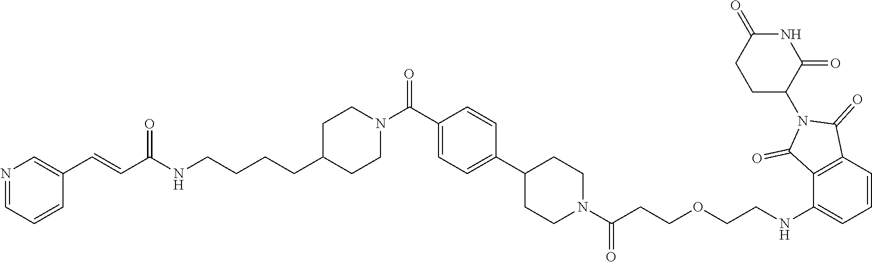 [SMILES: O=C(/C=C/c1cccnc1)NCCCCC1CCN(C(=O)c2ccc(C3CCN(C(=O)CCOCCNc4cccc5c4C(=O)N(C4CCC(=O)NC4=O)C5=O)CC3)cc2)CC1] | (E)-N-(4-(1-(4-(1-(3-(2- ((2-(2,6-dioxopiperidin- 3-yl)-1,3- dioxoisoindolin-4- yl)amino)ethoxy) propanoyl)piperidin-4- yl)benzoyl)piperidin-4- yl)butyl)-3-(pyridin-3- yl)acrylamide | (E)-N-(4-(1-(4-(1-(3- (2-((2-(2,6- dioxopiperidin-3-yl)- 1,3-dioxoisoindolin-4- yl)amino)ethoxy) propanoyl)piperidin-4- yl)benzoyl)piperidin-4- yl)butyl)-3-(pyridin-3- yl)acrylamide |
| | 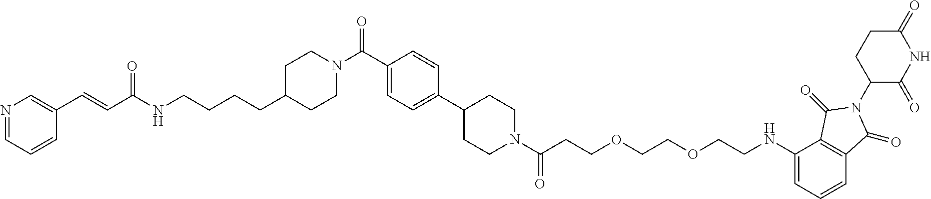 [SMILES: O=C(/C=C/c1cccnc1)NCCCCC1CCN(C(=O)c2ccc(C3CCN(C(=O)CCOCCOCCNc4cccc5c4C(=O)N(C4CCC(=O)NC4=O)C5=O)CC3)cc2)CC1] | (E)-N-(4-(1-(4-(1-(3-(2- (2-((2-(2,6- dioxopiperidin-3-yl)- 1,3-dioxoisoindolin-4- yl)amino)ethoxy)ethoxy) propanoyl)piperidin-4- yl)benzoyl)piperidin-4- yl)butyl)-3-(pyridin-3- yl)acrylamide | (E)-N-(4-(1-(4-(1-(3- (2-(2-((2-(2,6- dioxopiperidin-3-yl)- 1,3-dioxoisoindolin-4- yl)amino)ethoxy) ethoxy)propanoyl) piperidin- 4-yl)benzoyl)piperidin- 4-yl)butyl)-3-(pyridin- 3-yl)acrylamide |
| | 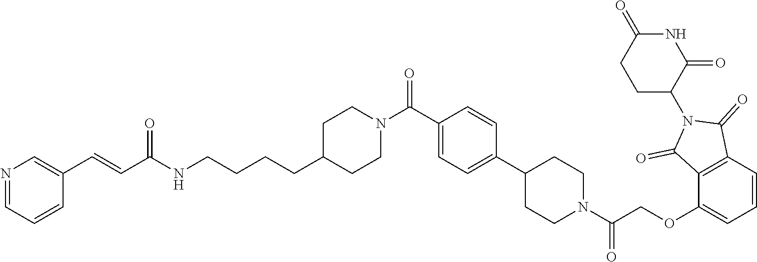 [SMILES: O=C(/C=C/c1cccnc1)NCCCCC1CCN(C(=O)c2ccc(C3CCN(C(=O)COc4cccc5c4C(=O)N(C4CCC(=O)NC4=O)C5=O)CC3)cc2)CC1] | (E)-N-(4-(1-(4-(1-(2- ((2-(2,6-dioxopiperidin- 3-yl)-1,3- dioxoisoindolin-4- yl)oxy)acetyl)piperidin- 4-yl)benzoyl)piperidin- 4-yl)butyl)-3-(pyridin-3- yl)acrylamide | (E)-N-(4-(1-(4-(1-(2- ((2-(2,6- dioxopiperidin-3-yl)- 1,3-dioxoisoindolin-4- yl)oxy)acetyl) piperidin-4- yl)benzoyl)piperidin-4- yl)butyl)-3-(pyridin-3- yl)acrylamide |
| | 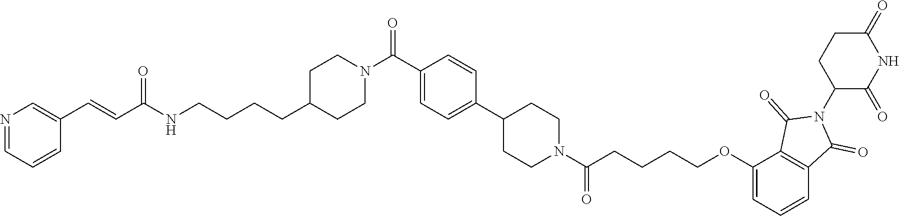 [SMILES: O=C(/C=C/c1cccnc1)NCCCCC1CCN(C(=O)c2ccc(C3CCN(C(=O)CCCCOc4cccc5c4C(=O)N(C4CCC(=O)NC4=O)C5=O)CC3)cc2)CC1] | (E)-N-(4-(1-(4-(1-(5- ((2-(2,6-dioxopiperidin- 3-yl)-1,3- dioxoisoindolin-4- yl)oxy)pentanoyl) piperidin-4- yl)benzoyl)piperidin-4- yl)butyl)-3-(pyridin-3- yl)acrylamide | (E)-N-(4-(1-(4-(1-(5- ((2-(2,6- dioxopiperidin-3-yl)- 1,3-dioxoisoindolin-4- yl)oxy)pentanoyl) piperidin-4- yl)benzoyl)piperidin-4- yl)butyl)-3-(pyridin-3- yl)acrylamide |
| | 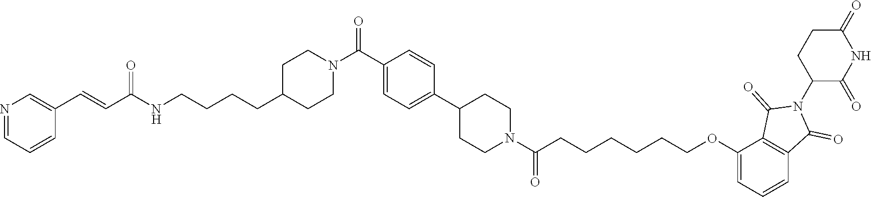 [SMILES: O=C(/C=C/c1cccnc1)NCCCCC1CCN(C(=O)c2ccc(C3CCN(C(=O)CCCCCCOc4cccc5c4C(=O)N(C4CCC(=O)NC4=O)C5=O)CC3)cc2)CC1] | (E)-N-(4-(1-(4-(1-(7- ((2-(2,6-dioxopiperidin- 3-yl)-1,3- dioxoisoindolin-4- yl)oxy)heptanoyl) piperidin-4- yl)benzoyl)piperidin-4- yl)butyl)-3-(pyridin-3- yl)acrylamide | (E)-N-(4-(1-(4-(1-(7- ((2-(2,6- dioxopiperidin-3-yl)- 1,3-dioxoisoindolin-4- yl)oxy)heptanoyl) piperidin-4- yl)benzoyl)piperidin-4- yl)butyl)-3-(pyridin-3- yl)acrylamide |
| | 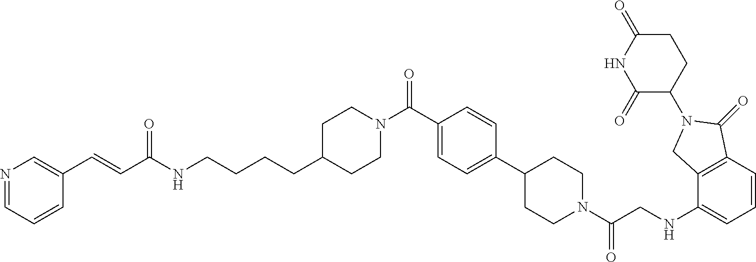 [SMILES: O=C(/C=C/c1cccnc1)NCCCCC1CCN(C(=O)c2ccc(C3CCN(C(=O)CNc4cccc5c4CN(C4CCC(=O)NC4=O)C5=O)CC3)cc2)CC1] | (E)-N-(4-(1-(4-(1-((2- (2,6-dioxopiperidin-3- yl)-1-oxoisoindolin-4- yl)glycyl)piperidin-4- yl)benzoyl)piperidin-4- yl)butyl)-3-(pyridin-3- yl)acrylamide | (E)-N-(4-(1-(4-(1-((2- (2,6-dioxopiperidin-3- yl)-1-oxoisoindolin-4- yl)glycyl)piperidin-4- yl)benzoyl)piperidin-4- yl)butyl)-3-(pyridin-3- yl)acrylamide |
| | 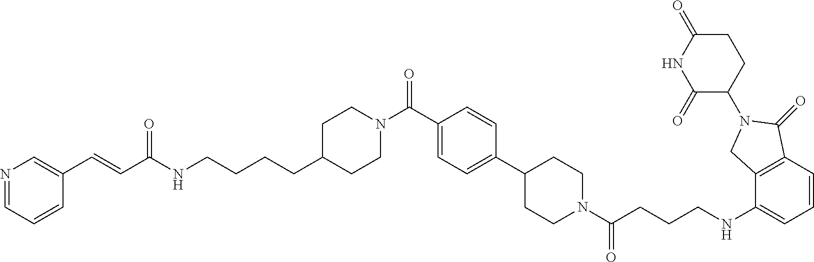 [SMILES: O=C(/C=C/c1cccnc1)NCCCCC1CCN(C(=O)c2ccc(C3CCN(C(=O)CCCNc4cccc5c4CN(C4CCC(=O)NC4=O)C5=O)CC3)cc2)CC1] | (E)-N-(4-(1-(4-(1-(4- ((2-(2,6-dioxopiperidin- 3-yl)-1-oxoisoindolin-4- yl)amino)butanoyl) piperidin-4- yl)benzoyl)piperidin-4- yl)butyl)-3-(pyridin-3- yl)acrylamide | (E)-N-(4-(1-(4-(1-(4- ((2-(2,6- dioxopiperidin-3-yl)-1- oxoisoindolin-4- yl)amino)butanoyl) piperidin-4- yl)benzoyl)piperidin-4- yl)butyl)-3-(pyridin-3- yl)acrylamide |
| | 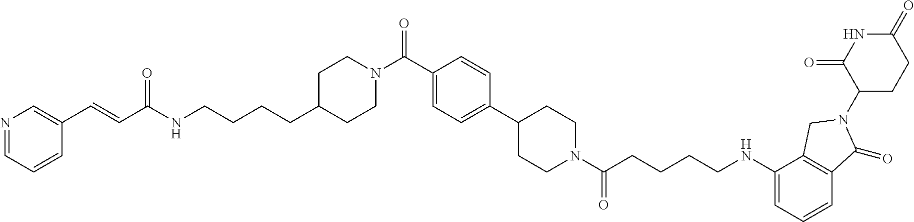 [SMILES: O=C(/C=C/c1cccnc1)NCCCCC1CCN(C(=O)c2ccc(C3CCN(C(=O)CCCCNc4cccc5c4CN(C4CCC(=O)NC4=O)C5=O)CC3)cc2)CC1] | (E)-N-(4-(1-(4-(1-(5- ((2-(2,6-dioxopiperidin- 3-yl)-1-oxoisoindolin-4- yl)amino)pentanoyl) piperidin-4- yl)benzoyl)piperidin-4- yl)butyl)-3-(pyridin-3- yl)acrylamide | (E)-N-(4-(1-(4-(1-(5- ((2-(2,6- dioxopiperidin-3-yl)-1- oxoisoindolin-4- yl)amino)pentanoyl) piperidin-4- yl)benzoyl)piperidin-4- yl)butyl)-3-(pyridin-3- yl)acrylamide |
| | 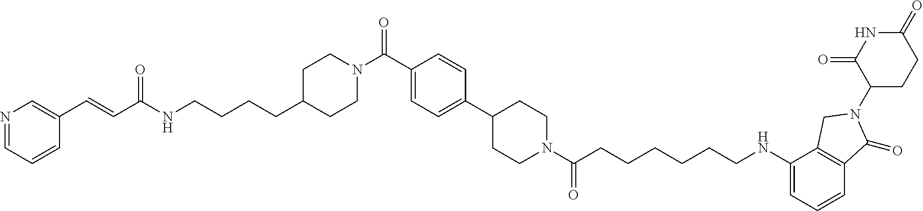 [SMILES: O=C(/C=C/c1cccnc1)NCCCCC1CCN(C(=O)c2ccc(C3CCN(C(=O)CCCCCCNc4cccc5c4CN(C4CCC(=O)NC4=O)C5=O)CC3)cc2)CC1] | (E)-N-(4-(1-(4-(1-(7- ((2-(2,6-dioxopiperidin- 3-yl)-1-oxoisoindolin-4- yl)amino)heptanoyl) piperidin-4- yl)benzoyl)piperidin-4- yl)butyl)-3-(pyridin-3- yl)acrylamide | (E)-N-(4-(1-(4-(1-(7- ((2-(2,6- dioxopiperidin-3-yl)-1- oxoisoindolin-4- yl)amino)heptanoyl) piperidin-4- yl)benzoyl)piperidin-4- yl)butyl)-3-(pyridin-3- yl)acrylamide |
| | 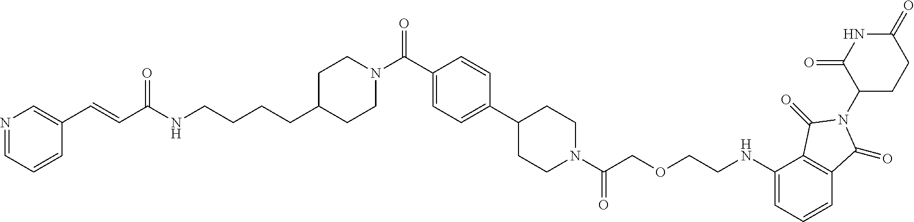 [SMILES: O=C(/C=C/c1cccnc1)NCCCCC1CCN(C(=O)c2ccc(C3CCN(C(=O)COCCNc4cccc5c4C(=O)N(C4CCC(=O)NC4=O)C5=O)CC3)cc2)CC1] | (E)-N-(4-(1-(4-(1-(2-(2- ((2-(2,6-dioxopiperidin- 3-yl)-1-oxoisoindolin-4- yl)amino)ethoxy)acetyl) piperidin-4- yl)benzoyl)piperidin-4- yl)butyl)-3-(pyridin-3- yl)acrylamide | (E)-N-(4-(1-(4-(1-(2- (2-((2-(2,6- dioxopiperidin-3-yl)-1- oxoisoindolin-4- yl)amino)ethoxy)acetyl) piperidin-4- yl)benzoyl)piperidin-4- yl)butyl)-3-(pyridin-3- yl)acrylamide |
| | 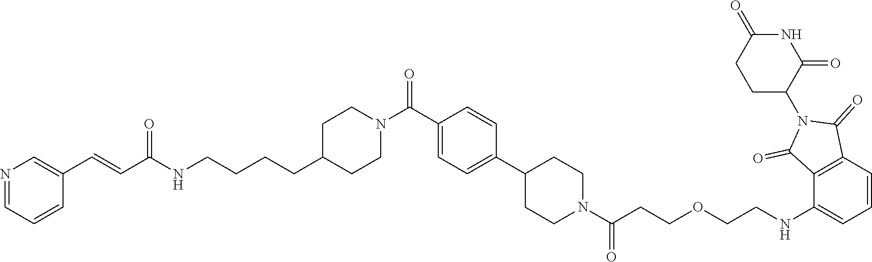 [SMILES: O=C(/C=C/c1cccnc1)NCCCCC1CCN(C(=O)c2ccc(C3CCN(C(=O)CCOCCNc4cccc5c4C(=O)N(C4CCC(=O)NC4=O)C5=O)CC3)cc2)CC1] | (E)-N-(4-(1-(4-(1-(3-(2- ((2-(2,6-dioxopiperidin- 3-yl)-1-oxoisoindolin-4- yl)amino)ethoxy) propanoyl)piperidin-4- yl)benzoyl)piperidin-4- yl)butyl)-3-(pyridin-3- yl)acrylamide | (E)-N-(4-(1-(4-(1-(3- (2-((2-(2,6- dioxopiperidin-3-yl)-1- oxoisoindolin-4- yl)amino)ethoxy) propanoyl)piperidin-4- yl)benzoyl)piperidin-4- yl)butyl)-3-(pyridin-3- yl)acrylamide |
| | 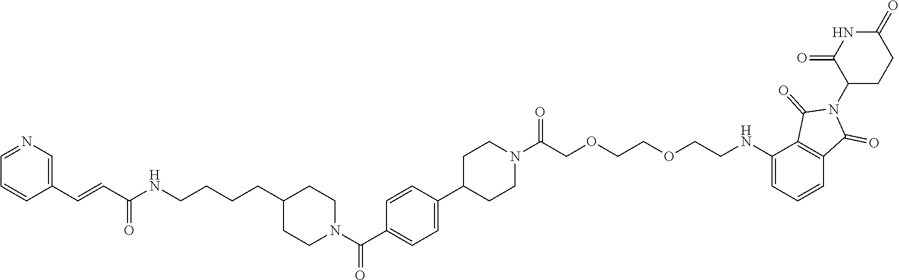 [SMILES: O=C(/C=C/c1cccnc1)NCCCCC1CCN(C(=O)c2ccc(C3CCN(C(=O)COCCOCCNc4cccc5c4C(=O)N(C4CCC(=O)NC4=O)C5=O)CC3)cc2)CC1] | (E)-N-(4-(1-(4-(1-(2-(2- (2-((2-(2,6- dioxopiperidin-3-yl)-1- oxoisoindolin-4- yl)amino)ethoxy)ethoxy) acetyl)piperidin-4- yl)benzoyl)piperidin-4- yl)butyl)-3-(pyridin-3- yl)acrylamide | (E)-N-(4-(1-(4-(1-(2- (2-(2-((2-(2,6- dioxopiperidin-3-yl)-1- oxoisoindolin-4- yl)amino)ethoxy) ethoxy)acetyl) piperidin-4- yl)benzoyl)piperidin-4- yl)butyl)-3-(pyridin-3- yl)acrylamide |
| | 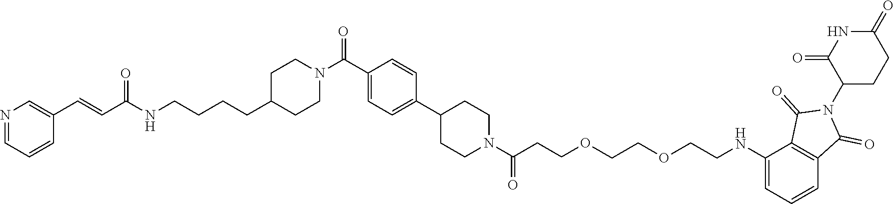 [SMILES: O=C(/C=C/c1cccnc1)NCCCCC1CCN(C(=O)c2ccc(C3CCN(C(=O)CCOCCOCCNc4cccc5c4C(=O)N(C4CCC(=O)NC4=O)C5=O)CC3)cc2)CC1] | (E)-N-(4-(1-(4-(1-(3-(2- (2-((2-(2,6- dioxopiperidin-3-yl)-1- oxoisoindolin-4- yl)amino)ethoxy)ethoxy) propanoyl)piperidin-4- yl)benzoyl)piperidin-4- yl)butyl)-3-(pyridin-3- yl)acrylamide | (E)-N-(4-(1-(4-(1-(3- (2-(2-((2-(2,6- dioxopiperidin-3-yl)-1- oxoisoindolin-4- yl)amino)ethoxy) ethoxy)propanoyl) piperidin- 4-yl)benzoyl)piperidin- 4-yl)butyl)-3-(pyridin- 3-yl)acrylamide |
| | 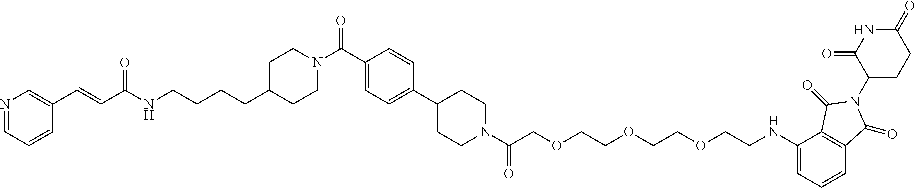 [SMILES: O=C(/C=C/c1cccnc1)NCCCCC1CCN(C(=O)c2ccc(C3CCN(C(=O)COCCOCCOCCNc4cccc5c4C(=O)N(C4CCC(=O)NC4=O)C5=O)CC3)cc2)CC1] | (E)-N-(4-(1-(4-(1-(2-(2- (2-(2-((2-(2,6- dioxopiperidin-3-yl)-1- oxoisoindolin-4- yl)amino)ethoxy)ethoxy) ethoxy)acetyl)piperidin- 4-yl)benzoyl)piperidin- 4-yl)butyl)-3-(pyridin-3- yl)acrylamide | (E)-N-(4-(1-(4-(1-(2- (2-(2-(2-((2-(2,6- dioxopiperidin-3-yl)-1- oxoisoindolin-4- yl)amino)ethoxy) ethoxy)ethoxy) acetyl)piperidin-4- yl)benzoyl)piperidin-4- yl)butyl)-3-(pyridin-3- yl)acrylamide |
| | 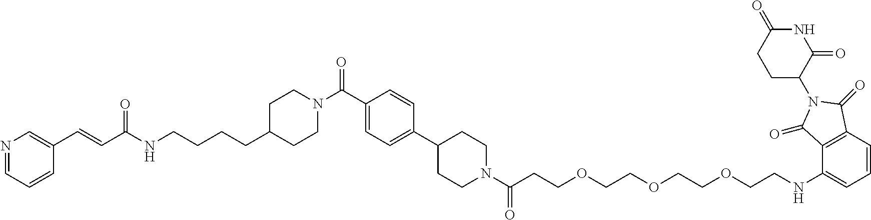 [SMILES: O=C(/C=C/c1cccnc1)NCCCCC1CCN(C(=O)c2ccc(C3CCN(C(=O)CCOCCOCCOCCNc4cccc5c4C(=O)N(C4CCC(=O)NC4=O)C5=O)CC3)cc2)CC1] | (E)-N-(4-(1-(4-(1-(3-(2- (2-(2-((2-(2,6- dioxopiperidin-3-yl)-1- oxoisoindolin-4- yl)amino)ethoxy)ethoxy) ethoxy)propanoyl) piperidin-4- yl)benzoyl)piperidin-4- yl)butyl)-3-(pyridin-3- yl)acrylamide | (E)-N-(4-(1-(4-(1-(3- (2-(2-(2-((2-(2,6- dioxopiperidin-3-yl)-1- oxoisoindolin-4- yl)amino)ethoxy) ethoxy)ethoxy) propanoyl)piperidin-4- yl)benzoyl)piperidin-4- yl)butyl)-3-(pyridin-3- yl)acrylamide |
| | 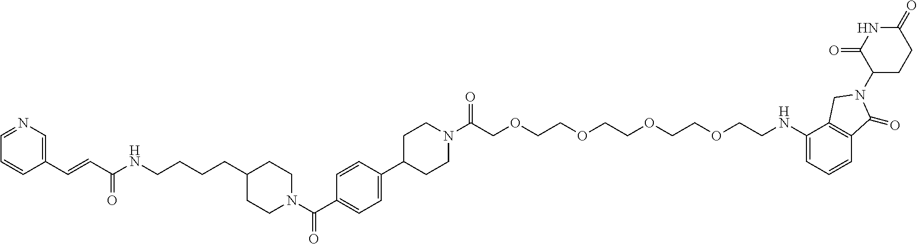 [SMILES: O=C(/C=C/c1cccnc1)NCCCCC1CCN(C(=O)c2ccc(C3CCN(C(=O)COCCOCCOCCOCCNc4cccc5c4CN(C4CCC(=O)NC4=O)C5=O)CC3)cc2)CC1] | (E)-N-(4-(1-(4-(1-(14- ((2-(2,6-dioxopiperidin- 3-yl)-1-oxoisoindolin-4- yl)amino)-3,6,9,12- tetraoxatetradecanoyl) piperidin-4- yl)benzoyl)piperidin-4- yl)butyl)-3-(pyridin-3- yl)acrylamide | (E)-N-(4-(1-(4-(1-(14- ((2-(2,6- dioxopiperidin-3-yl)-1- oxoisoindolin-4- yl)amino)-3,6,9,12- tetraoxatetradecanoyl) piperidin-4- yl)benzoyl)piperidin-4- yl)butyl)-3-(pyridin-3- yl)acrylamide |
| | 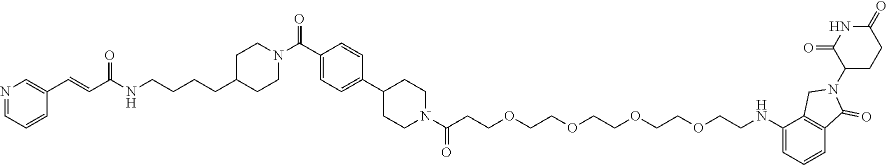 [SMILES: O=C(/C=C/c1cccnc1)NCCCCC1CCN(C(=O)c2ccc(C3CCN(C(=O)CCOCCOCCOCCOCCNc4cccc5c4CN(C4CCC(=O)NC4=O)C5=O)CC3)cc2)CC1] | (E)-N-(4-(1-(4-(1-(1- ((2-(2,6-dioxopiperidin- 3-yl)-1-oxoisoindolin-4- yl)amino)-3,6,9,12- tetraoxapentadecan-15- oyl)piperidin-4- yl)benzoyl)piperidin-4- yl)butyl)-3-(pyridin-3- yl)acrylamide | (E)-N-(4-(1-(4-(1-(1- ((2-(2,6- dioxopiperidin-3-yl)-1- oxoisoindolin-4- yl)amino)-3,6,9,12- tetraoxapentadecan-15- oyl)piperidin-4- yl)benzoyl)piperidin-4- yl)butyl)-3-(pyridin-3- yl)acrylamide |
| | 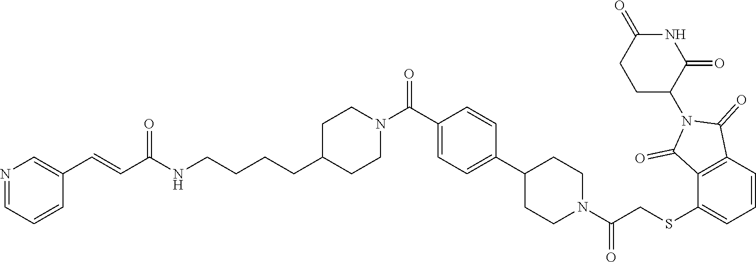 [SMILES: O=C(/C=C/c1cccnc1)NCCCCC1CCN(C(=O)c2ccc(C3CCN(C(=O)CSc4cccc5c4C(=O)N(C4CCC(=O)NC4=O)C5=O)CC3)cc2)CC1] | (E)-N-(4-(1-(4-(1-(2- ((2-(2,6-dioxopiperidin- 3-yl)-1-oxoisoindolin-4- yl)thio)acetyl)piperidin- 4-yl)benzoyl)piperidin- 4-yl)butyl)-3-(pyridin-3- yl)acrylamide | (E)-N-(4-(1-(4-(1-(2- ((2-(2,6- dioxopiperidin-3-yl)-1- oxoisoindolin-4- yl)thio)acetyl) piperidin-4- yl)benzoyl)piperidin-4- yl)butyl)-3-(pyridin-3- yl)acrylamide |
| | 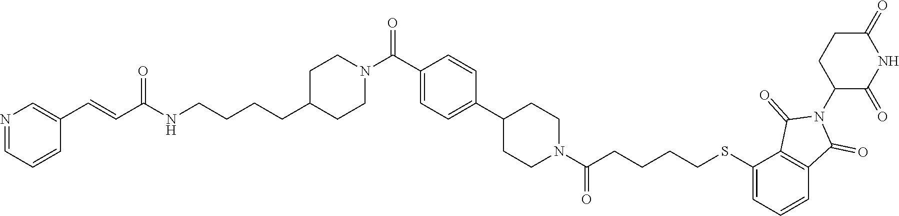 [SMILES: O=C(/C=C/c1cccnc1)NCCCCC1CCN(C(=O)c2ccc(C3CCN(C(=O)CCCCSc4cccc5c4C(=O)N(C4CCC(=O)NC4=O)C5=O)CC3)cc2)CC1] | (E)-N-(4-(1-(4-(1-(5- ((2-(2,6-dioxopiperidin- 3-yl)-1-oxoisoindolin-4- yl)thio)pentanoyl) piperidin-4- yl)benzoyl)piperidin-4- yl)butyl)-3-(pyridin-3- yl)acrylamide | (E)-N-(4-(1-(4-(1-(5- ((2-(2,6- dioxopiperidin-3-yl)-1- oxoisoindolin-4- yl)thio)pentanoyl) piperidin-4- yl)benzoyl)piperidin-4- yl)butyl)-3-(pyridin-3- yl)acrylamide |
| | 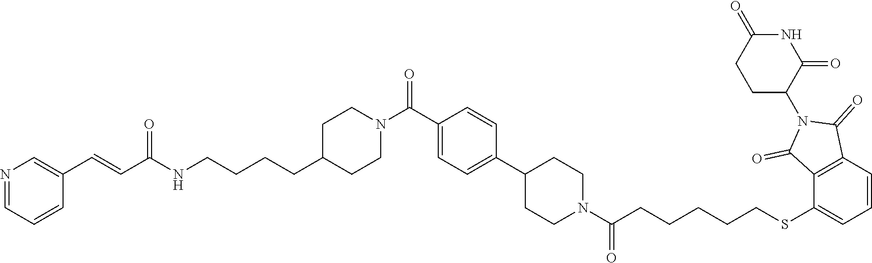 [SMILES: O=C(/C=C/c1cccnc1)NCCCCC1CCN(C(=O)c2ccc(C3CCN(C(=O)CCCCCSc4cccc5c4C(=O)N(C4CCC(=O)NC4=O)C5=O)CC3)cc2)CC1] | (E)-N-(4-(1-(4-(1-(6- ((2-(2,6-dioxopiperidin- 3-yl)-1-oxoisoindolin-4- yl)thio)hexanoyl) piperidin-4- yl)benzoyl)piperidin-4- yl)butyl)-3-(pyridin-3- yl)acrylamide | (E)-N-(4-(1-(4-(1-(6- ((2-(2,6- dioxopiperidin-3-yl)-1- oxoisoindolin-4- yl)thio)hexanoyl) piperidin-4- yl)benzoyl)piperidin-4- yl)butyl)-3-(pyridin-3- yl)acrylamide |
| | 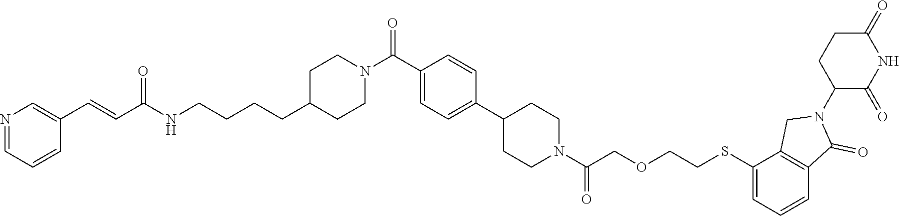 [SMILES: O=C(/C=C/c1cccnc1)NCCCCC1CCN(C(=O)c2ccc(C3CCN(C(=O)COCCSc4cccc5c4CN(C4CCC(=O)NC4=O)C5=O)CC3)cc2)CC1] | (E)-N-(4-(1-(4-(1-(2-(2- ((2-(2,6-dioxopiperidin- 3-yl)-1-oxoisoindolin-4- yl)thio)ethoxy)acetyl) piperidin-4- yl)benzoyl)piperidin-4- yl)butyl)-3-(pyridin-3- yl)acrylamide | (E)-N-(4-(1-(4-(1-(2- (2-((2-(2,6- dioxopiperidin-3-yl)-1- oxoisoindolin-4- yl)thio)ethoxy)acetyl) piperidin-4- yl)benzoyl)piperidin-4- yl)butyl)-3-(pyridin-3- yl)acrylamide |
| | 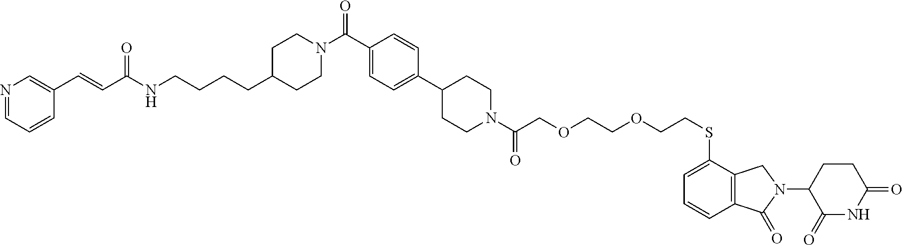 [SMILES: O=C(/C=C/c1cccnc1)NCCCCC1CCN(C(=O)c2ccc(C3CCN(C(=O)COCCOCCSc4cccc5c4CN(C4CCC(=O)NC4=O)C5=O)CC3)cc2)CC1] | (E)-N-(4-(1-(4-(1-(2-(2- (2-((2-(2,6- dioxopiperidin-3-yl)-1- oxoisoindolin-4- yl)thio)ethoxy)ethoxy) acetyl)piperidin-4- yl)benzoyl)piperidin-4- yl)butyl)-3-(pyridin-3- yl)acrylamide | (E)-N-(4-(1-(4-(1-(2- (2-(2-((2-(2,6- dioxopiperidin-3-yl)-1- oxoisoindolin-4- yl)thio)ethoxy)ethoxy) acetyl)piperidin-4- yl)benzoyl)piperidin-4- yl)butyl)-3-(pyridin-3- yl)acrylamide |
| | 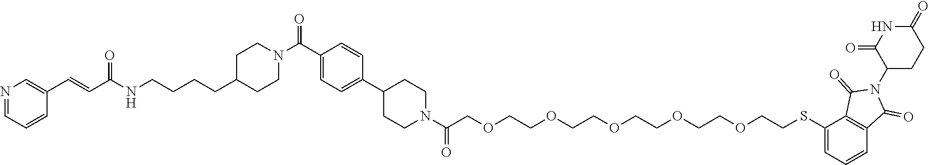 [SMILES: O=C(/C=C/c1cccnc1)NCCCCC1CCN(C(=O)c2ccc(C3CCN(C(=O)COCCOCCOCCOCCOCCSc4cccc5c4C(=O)N(C4CCC(=O)NC4=O)C5=O)CC3)cc2)CC1] | (E)-N-(4-(1-(4-(1-(17- ((2-(2,6-dioxopiperidin- 3-yl)-1-oxoisoindolin-4- yl)thio)-3,6,9,12,15- pentaoxaheptadecanoyl) piperidin-4- yl)benzoyl)piperidin-4- yl)butyl)-3-(pyridin-3- yl)acrylamide | (E)-N-(4-(1-(4-(1-(17- ((2-(2,6- dioxopiperidin-3-yl)-1- oxoisoindolin-4- yl)thio)-3,6,9,12,15- pentaoxaheptadecanoyl )piperidin-4- yl)benzoyl)piperidin-4- yl)butyl)-3-(pyridin-3- yl)acrylamide |
| | 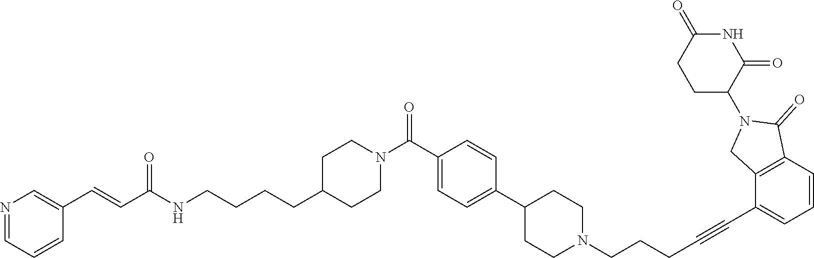 [SMILES: O=C(/C=C/c1cccnc1)NCCCCC1CCN(C(=O)c2ccc(C3CCN(CCCC#Cc4cccc5c4CN(C4CCC(=O)NC4=O)C5=O)CC3)cc2)CC1] | (E)-N-(4-(1-(4-(1-(5-(2- (2,6-dioxopiperidin-3- yl)-1-oxoisoindolin-4- yl)pent-4-yn-1- yl)piperidin-4- yl)benzoyl)piperidin-4- yl)butyl)-3-(pyridin-3- yl)acrylamide | (E)-N-(4-(1-(4-(1-(5- (2-(2,6-dioxopiperidin- 3-yl)-1-oxoisoindolin- 4-yl)pent-4-yn-1- yl)piperidin-4- yl)benzoyl)piperidin-4- yl)butyl)-3-(pyridin-3- yl)acrylamide |
| | 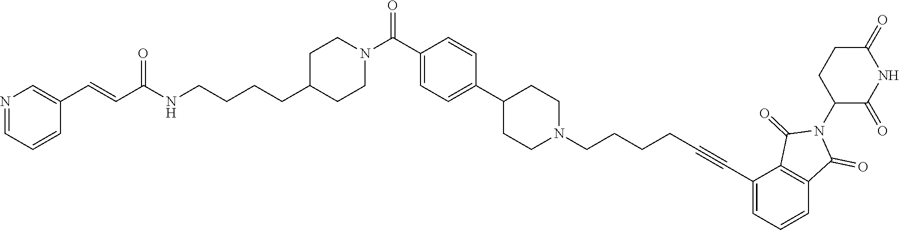 [SMILES: O=C(/C=C/c1cccnc1)NCCCCC1CCN(C(=O)c2ccc(C3CCN(CCCCC#Cc4cccc5c4C(=O)N(C4CCC(=O)NC4=O)C5=O)CC3)cc2)CC1] | (E)-N-(4-(1-(4-(1-(6-(2- (2,6-dioxopiperidin-3- yl)-1-oxoisoindolin-4- yl)hex-5-yn-1- yl)piperidin-4- yl)benzoyl)piperidin-4- yl)butyl)-3-(pyridin-3- yl)acrylamide | (E)-N-(4-(1-(4-(1-(6- (2-(2,6-dioxopiperidin- 3-yl)-1-oxoisoindolin- 4-yl)hex-5-yn-1- yl)piperidin-4- yl)benzoyl)piperidin-4- yl)butyl)-3-(pyridin-3- yl)acrylamide |
| | 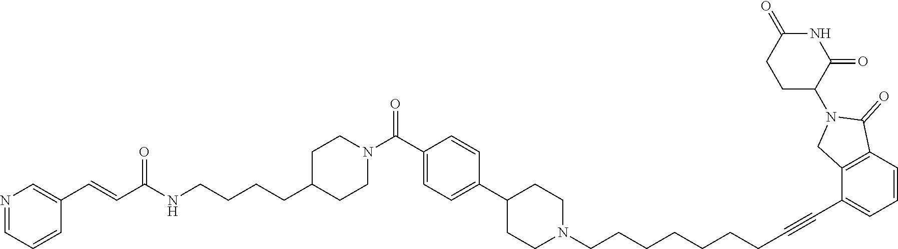 [SMILES: O=C(/C=C/c1cccnc1)NCCCCC1CCN(C(=O)c2ccc(C3CCN(CCCCCCCC#Cc4cccc5c4CN(C4CCC(=O)NC4=O)C5=O)CC3)cc2)CC1] | (E)-N-(4-(1-(4-(1-(9-(2- (2,6-dioxopiperidin-3- yl)-1-oxoisoindolin-4- yl)non-8-yn-1- yl)piperidin-4- yl)benzoyl)piperidin-4- yl)butyl)-3-(pyridin-3- yl)acrylamide | (E)-N-(4-(1-(4-(1-(9- (2-(2,6-dioxopiperidin- 3-yl)-1-oxoisoindolin- 4-yl)non-8-yn-1- yl)piperidin-4- yl)benzoyl)piperidin-4- yl)butyl)-3-(pyridin-3- yl)acrylamide |
| | 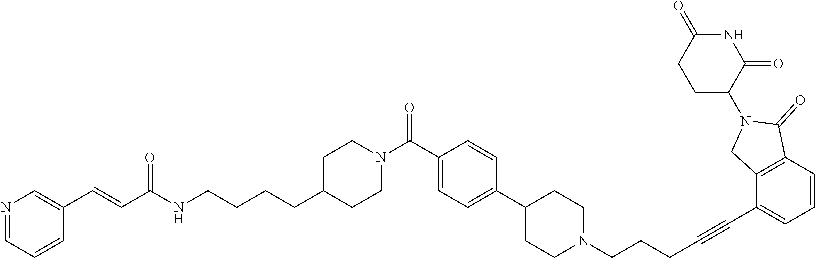 [SMILES: O=C(/C=C/c1cccnc1)NCCCCC1CCN(C(=O)c2ccc(C3CCN(CCCC#Cc4cccc5c4CN(C4CCC(=O)NC4=O)C5=O)CC3)cc2)CC1] | (E)-N-(4-(1-(4-(1-(5-(2- (2,6-dioxopiperidin-3- yl)-1,3-dioxoisoindolin- 4-yl)pent-4-yn-1- yl)piperidin-4- yl)benzoyl)piperidin-4- yl)butyl)-3-(pyridin-3- yl)acrylamide | (E)-N-(4-(1-(4-(1-(5- (2-(2,6-dioxopiperidin- 3-yl)-1,3- dioxoisoindolin-4- yl)pent-4-yn-1- yl)piperidin-4- yl)benzoyl)piperidin-4- yl)butyl)-3-(pyridin-3- yl)acrylamide |
| | 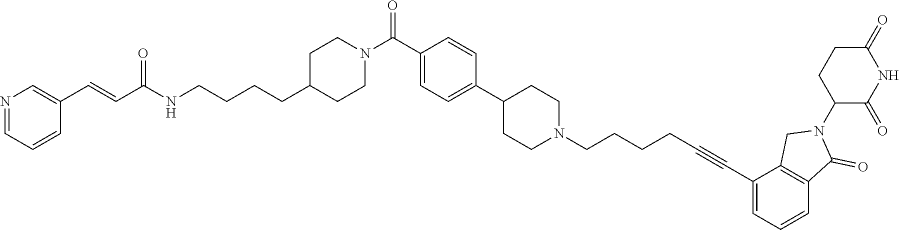 [SMILES: O=C(/C=C/c1cccnc1)NCCCCC1CCN(C(=O)c2ccc(C3CCN(CCCCC#Cc4cccc5c4CN(C4CCC(=O)NC4=O)C5=O)CC3)cc2)CC1] | (E)-N-(4-(1-(4-(1-(6-(2- (2,6-dioxopiperidin-3- yl)-1,3-dioxoisoindolin- 4-yl)hex-5-yn-1- yl)piperidin-4- yl)benzoyl)piperidin-4- yl)butyl)-3-(pyridin-3- yl)acrylamide | (E)-N-(4-(1-(4-(1-(6- (2-(2,6-dioxopiperidin- 3-yl)-1,3- dioxoisoindolin-4- yl)hex-5-yn-1- yl)piperidin-4- yl)benzoyl)piperidin-4- yl)butyl)-3-(pyridin-3- yl)acrylamide |
| | 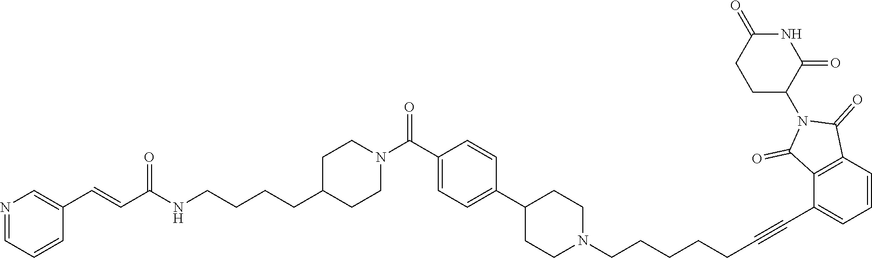 [SMILES: O=C(/C=C/c1cccnc1)NCCCCC1CCN(C(=O)c2ccc(C3CCN(CCCCCC#Cc4cccc5c4C(=O)N(C4CCC(=O)NC4=O)C5=O)CC3)cc2)CC1] | (E)-N-(4-(1-(4-(1-(7-(2- (2,6-dioxopiperidin-3- yl)-1,3-dioxoisoindolin- 4-yl)hept-6-yn-1- yl)piperidin-4- yl)benzoyl)piperidin-4- yl)butyl)-3-(pyridin-3- yl)acrylamide | (E)-N-(4-(1-(4-(1-(7- (2-(2,6-dioxopiperidin- 3-yl)-1,3- dioxoisoindolin-4- yl)hept-6-yn-1- yl)piperidin-4- yl)benzoyl)piperidin-4- yl)butyl)-3-(pyridin-3- yl)acrylamide |
| | 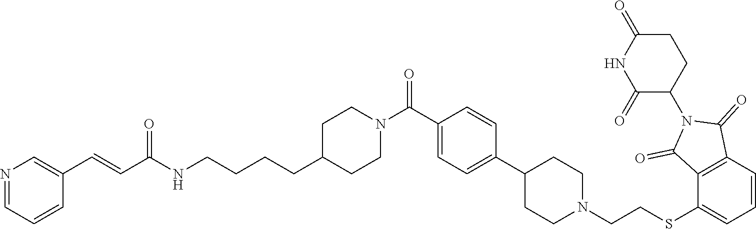 [SMILES: O=C(/C=C/c1cccnc1)NCCCCC1CCN(C(=O)c2ccc(C3CCN(CCSc4cccc5c4C(=O)N(C4CCC(=O)NC4=O)C5=O)CC3)cc2)CC1] | (E)-N-(4-(1-(4-(1-(2- ((2-(2,6-dioxopiperidin- 3-yl)-1-oxoisoindolin-4- yl)thio)ethyl)piperidin- 4-yl)benzoyl)piperidin- 4-yl)butyl)-3-(pyridin-3- yl)acrylamide | (E)-N-(4-(1-(4-(1-(2- ((2-(2,6- dioxopiperidin-3-yl)-1- oxoisoindolin-4- yl)thio)ethyl)piperidin- 4-yl)benzoyl)piperidin- 4-yl)butyl)-3-(pyridin- 3-yl)acrylamide |
| | 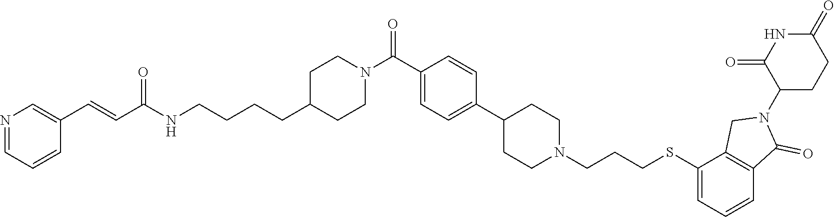 [SMILES: O=C(/C=C/c1cccnc1)NCCCCC1CCN(C(=O)c2ccc(C3CCN(CCCSc4cccc5c4CN(C4CCC(=O)NC4=O)C5=O)CC3)cc2)CC1] | (E)-N-(4-(1-(4-(1-(3- ((2-(2,6-dioxopiperidin- 3-yl)-1-oxoisoindolin-4- yl)thio)propyl)piperidin- 4-yl)benzoyl)piperidin- 4-yl)butyl)-3-(pyridin-3- yl)acrylamide | (E)-N-(4-(1-(4-(1-(3- ((2-(2,6- dioxopiperidin-3-yl)-1- oxoisoindolin-4- yl)thio)propyl) piperidin-4- yl)benzoyl)piperidin-4- yl)butyl)-3-(pyridin-3- yl)acrylamide |
| | 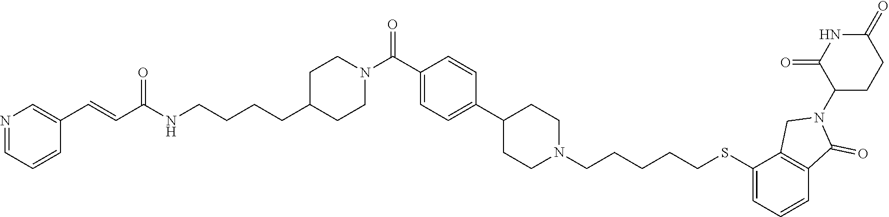 [SMILES: O=C(/C=C/c1cccnc1)NCCCCC1CCN(C(=O)c2ccc(C3CCN(CCCCCSc4cccc5c4CN(C4CCC(=O)NC4=O)C5=O)CC3)cc2)CC1] | (E)-N-(4-(1-(4-(1-(5- ((2-(2,6-dioxopiperidin- 3-yl)-1-oxoisoindolin-4- yl)thio)pentyl)piperidin- 4-yl)benzoyl)piperidin- 4-yl)butyl)-3-(pyridin-3- yl)acrylamide | (E)-N-(4-(1-(4-(1-(5- ((2-(2,6- dioxopiperidin-3-yl)-1- oxoisoindolin-4- yl)thio)pentyl) piperidin-4- yl)benzoyl)piperidin-4- yl)butyl)-3-(pyridin-3- yl)acrylamide |
| | 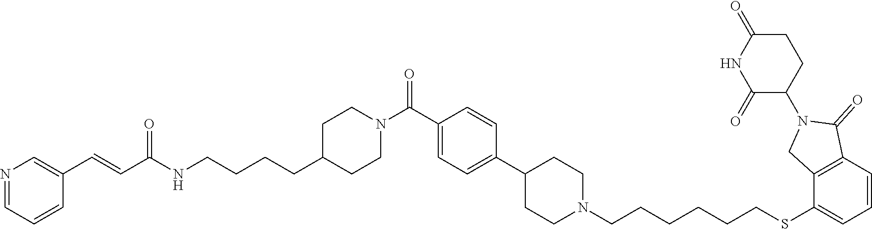 [SMILES: O=C(/C=C/c1cccnc1)NCCCCC1CCN(C(=O)c2ccc(C3CCN(CCCCCCSc4cccc5c4CN(C4CCC(=O)NC4=O)C5=O)CC3)cc2)CC1] | (E)-N-(4-(1-(4-(1-(6- ((2-(2,6-dioxopiperidin- 3-yl)-1-oxoisoindolin-4- yl)thio)hexyl)piperidin- 4-yl)benzoyl)piperidin- 4-yl)butyl)-3-(pyridin-3- yl)acrylamide | (E)-N-(4-(1-(4-(1-(6- ((2-(2,6- dioxopiperidin-3-yl)-1- oxoisoindolin-4- yl)thio)hexyl)piperidin- 4-yl)benzoyl)piperidin- 4-yl)butyl)-3-(pyridin- 3-yl)acrylamide |
| | 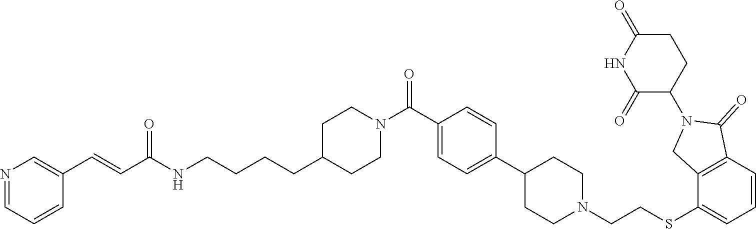 [SMILES: O=C(/C=C/c1cccnc1)NCCCCC1CCN(C(=O)c2ccc(C3CCN(CCSc4cccc5c4CN(C4CCC(=O)NC4=O)C5=O)CC3)cc2)CC1] | (E)-N-(4-(1-(4-(1-(2- ((2-(2,6-dioxopiperidin- 3-yl)-1,3- dioxoisoindolin-4- yl)thio)ethyl)piperidin- 4-yl)benzoyl)piperidin- 4-yl)butyl)-3-(pyridin-3- yl)acrylamide | (E)-N-(4-(1-(4-(1-(2- ((2-(2,6- dioxopiperidin-3-yl)- 1,3-dioxoisoindolin-4- yl)thio)ethyl)piperidin- 4-yl)benzoyl)piperidin- 4-yl)butyl)-3-(pyridin- 3-yl)acrylamide |
| | 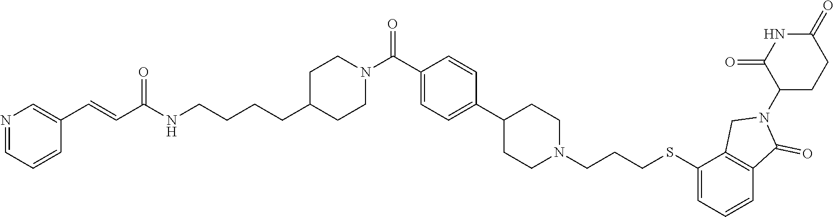 [SMILES: O=C(/C=C/c1cccnc1)NCCCCC1CCN(C(=O)c2ccc(C3CCN(CCCSc4cccc5c4CN(C4CCC(=O)NC4=O)C5=O)CC3)cc2)CC1] | (E)-N-(4-(1-(4-(1-(3- ((2-(2,6-dioxopiperidin- 3-yl)-1,3- dioxoisoindolin-4- yl)thio)propyl)piperidin- 4-yl)benzoyl)piperidin- 4-yl)butyl)-3-(pyridin-3- yl)acrylamide | (E)-N-(4-(1-(4-(1-(3- ((2-(2,6- dioxopiperidin-3-yl)- 1,3-dioxoisoindolin-4- yl)thio)propyl) piperidin-4- yl)benzoyl)piperidin-4- yl)butyl)-3-(pyridin-3- yl)acrylamide |
| | 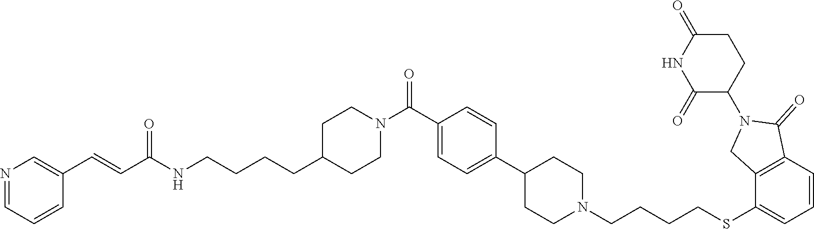 [SMILES: O=C(/C=C/c1cccnc1)NCCCCC1CCN(C(=O)c2ccc(C3CCN(CCCCSc4cccc5c4CN(C4CCC(=O)NC4=O)C5=O)CC3)cc2)CC1] | (E)-N-(4-(1-(4-(1-(4- ((2-(2,6-dioxopiperidin- 3-yl)-1,3- dioxoisoindolin-4- yl)thio)butyl)piperidin- 4-yl)benzoyl)piperidin- 4-yl)butyl)-3-(pyridin-3- yl)acrylamide | (E)-N-(4-(1-(4-(1-(4- ((2-(2,6- dioxopiperidin-3-yl)- 1,3-dioxoisoindolin-4- yl)thio)butyl)piperidin- 4-yl)benzoyl)piperidin- 4-yl)butyl)-3-(pyridin- 3-yl)acrylamide |
| | 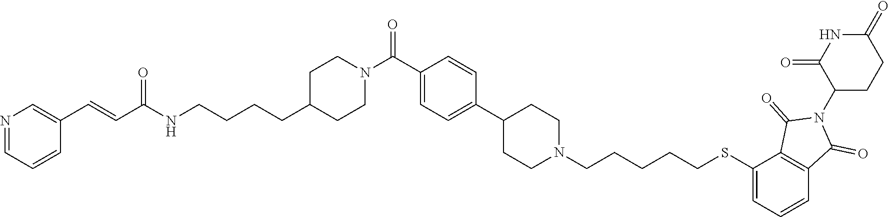 [SMILES: O=C(/C=C/c1cccnc1)NCCCCC1CCN(C(=O)c2ccc(C3CCN(CCCCCSc4cccc5c4C(=O)N(C4CCC(=O)NC4=O)C5=O)CC3)cc2)CC1] | (E)-N-(4-(1-(4-(1-(5- ((2-(2,6-dioxopiperidin- 3-yl)-1,3- dioxoisoindolin-4- yl)thio)pentyl)piperidin- 4-yl)benzoyl)piperidin- 4-yl)butyl)-3-(pyridin-3- yl)acrylamide | (E)-N-(4-(1-(4-(1-(5- ((2-(2,6- dioxopiperidin-3-yl)- 1,3-dioxoisoindolin-4- yl)thio)pentyl) piperidin-4- yl)benzoyl)piperidin-4- yl)butyl)-3-(pyridin-3- yl)acrylamide |
| | 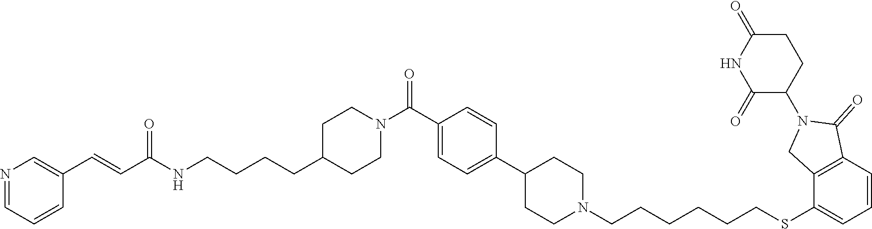 [SMILES: O=C(/C=C/c1cccnc1)NCCCCC1CCN(C(=O)c2ccc(C3CCN(CCCCCCSc4cccc5c4CN(C4CCC(=O)NC4=O)C5=O)CC3)cc2)CC1] | (E)-N-(4-(1-(4-(1-(6- ((2-(2,6-dioxopiperidin- 3-yl)-1,3- dioxoisoindolin-4- yl)thio)hexyl)piperidin- 4-yl)benzoyl)piperidin- 4-yl)butyl)-3-(pyridin-3- yl)acrylamide | (E)-N-(4-(1-(4-(1-(6- ((2-(2,6- dioxopiperidin-3-yl)- 1,3-dioxoisoindolin-4- yl)thio)hexyl)piperidin- 4-yl)benzoyl)piperidin- 4-yl)butyl)-3-(pyridin- 3-yl)acrylamide |
| | 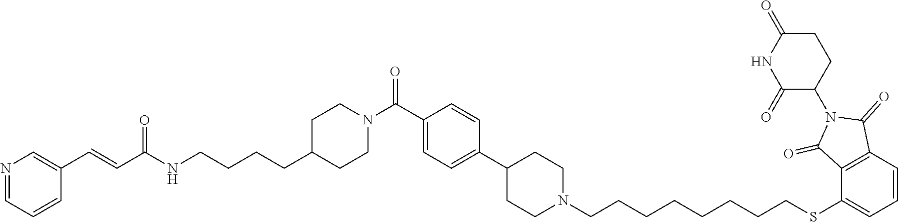 [SMILES: O=C(/C=C/c1cccnc1)NCCCCC1CCN(C(=O)c2ccc(C3CCN(CCCCCCCCSc4cccc5c4C(=O)N(C4CCC(=O)NC4=O)C5=O)CC3)cc2)CC1] | (E)-N-(4-(1-(4-(1-(8- ((2-(2,6-dioxopiperidin- 3-yl)-1,3- dioxoisoindolin-4- yl)thio)octyl)piperidin- 4-yl)benzoyl)piperidin- 4-yl)butyl)-3-(pyridin-3- yl)acrylamide | (E)-N-(4-(1-(4-(1-(8- ((2-(2,6- dioxopiperidin-3-yl)- 1,3-dioxoisoindolin-4- yl)thio)octyl)piperidin- 4-yl)benzoyl)piperidin- 4-yl)butyl)-3-(pyridin- 3-yl)acrylamide |
| | 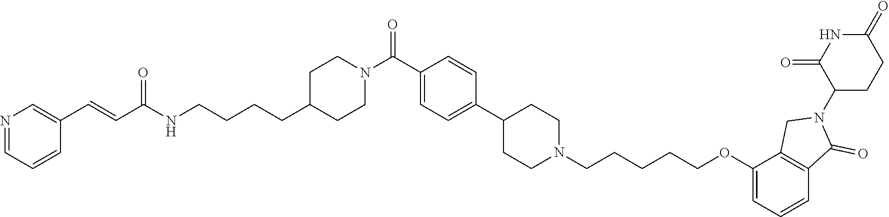 [SMILES: O=C(/C=C/c1cccnc1)NCCCCC1CCN(C(=O)c2ccc(C3CCN(CCCCCOc4cccc5c4CN(C4CCC(=O)NC4=O)C5=O)CC3)cc2)CC1] | (E)-N-(4-(1-(4-(1-(5- ((2-(2,6-dioxopiperidin- 3-yl)-1-oxoisoindolin-4- yl)oxy)pentyl)piperidin- 4-yl)benzoyl)piperidin- 4-yl)butyl)-3-(pyridin-3- yl)acrylamide | (E)-N-(4-(1-(4-(1-(5- ((2-(2,6- dioxopiperidin-3-yl)-1 - oxoisoindolin-4- yl)oxy)pentyl) piperidin-4- yl)benzoyl)piperidin-4- yl)butyl)-3-(pyridin-3- yl)acrylamide |
| | 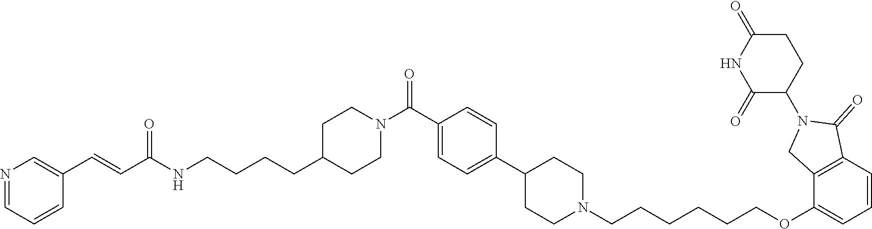 [SMILES: O=C(/C=C/c1cccnc1)NCCCCC1CCN(C(=O)c2ccc(C3CCN(CCCCCCOc4cccc5c4CN(C4CCC(=O)NC4=O)C5=O)CC3)cc2)CC1] | (E)-N-(4-(1-(4-(1-(6- ((2-(2,6-dioxopiperidin- 3-yl)-1-oxoisoindolin-4- yl)oxy)hexyl)piperidin- 4-yl)benzoyl)piperidin- 4-yl)butyl)-3-(pyridin-3- yl)acrylamide | (E)-N-(4-(1-(4-(1-(6- ((2-(2,6- dioxopiperidin-3-yl)-1- oxoisoindolin-4- yl)oxy)hexyl)piperidin- 4-yl)benzoyl)piperidin- 4-yl)butyl)-3-(pyridin- 3-yl)acrylamide |
| | 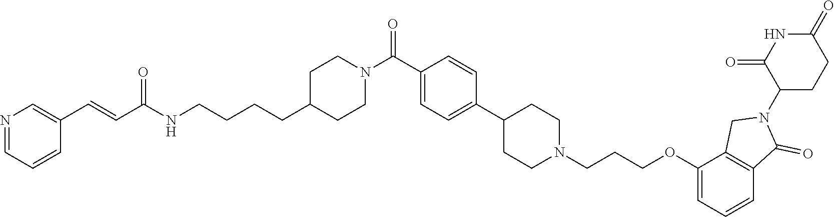 [SMILES: O=C(/C=C/c1cccnc1)NCCCCC1CCN(C(=O)c2ccc(C3CCN(CCCOc4cccc5c4CN(C4CCC(=O)NC4=O)C5=O)CC3)cc2)CC1] | (E)-N-(4-(1-(4-(1-(3- ((2-(2,6-dioxopiperidin- 3-yl)-1,3- dioxoisoindolin-4- yl)oxy)propyl)piperidin- 4-yl)benzoyl)piperidin- 4-yl)butyl)-3-(pyridin-3- yl)acrylamide | (E)-N-(4-(1-(4-(1-(3- ((2-(2,6- dioxopiperidin-3-yl)- 1,3-dioxoisoindolin-4- yl)oxy)propyl) piperidin-4- yl)benzoyl)piperidin-4- yl)butyl)-3-(pyridin-3- yl)acrylamide |
| | 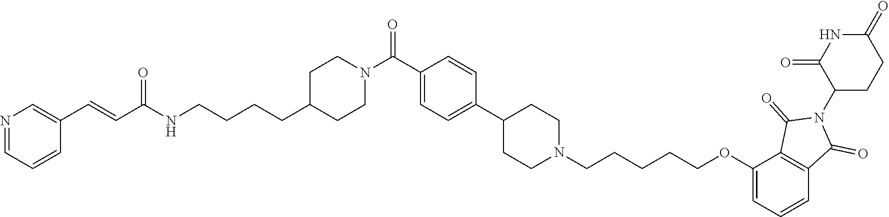 [SMILES: O=C(/C=C/c1cccnc1)NCCCCC1CCN(C(=O)c2ccc(C3CCN(CCCCCOc4cccc5c4C(=O)N(C4CCC(=O)NC4=O)C5=O)CC3)cc2)CC1] | (E)-N-(4-(1-(4-(1-(5- ((2-(2,6-dioxopiperidin- 3-yl)-1,3- dioxoisoindolin-4- yl)oxy)pentyl)piperidin- 4-yl)benzoyl)piperidin- 4-yl)butyl)-3-(pyridin-3- yl)acrylamide | (E)-N-(4-(1-(4-(1-(5- ((2-(2,6- dioxopiperidin-3-yl)- 1,3-dioxoisoindolin-4- yl)oxy)pentyl) piperidin-4- yl)benzoyl)piperidin-4- yl)butyl)-3-(pyridin-3- yl)acrylamide |
| | 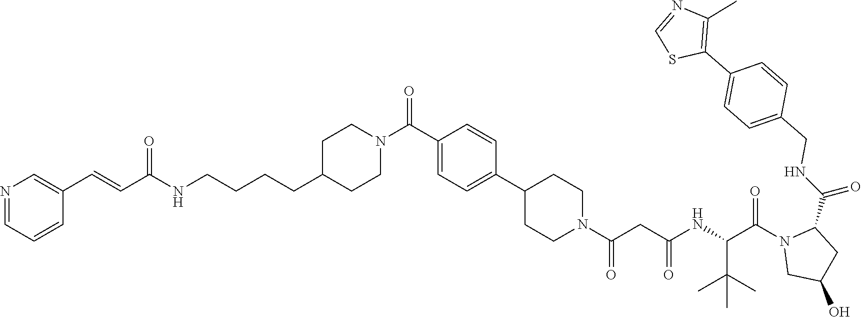 [SMILES: Cc1ncsc1-c1ccc(CNC(=O)[C@@H]2C[C@@H](O)CN2C(=O)[C@@H](NC(=O)CC(=O)N2CCC(c3ccc(C(=O)N4CCC(CCCCNC(=O)/C=C/c5cccnc5)CC4)cc3)CC2)C(C)(C)C)cc1] | (2S,4R)-1-((S)-3,3- dimethyl-2-(3-oxo-3-(4- (4-(4-(4-(E)-3- (pyridin-3- yl)acrylamido)butyl) piperidine-1- carbonyl)phenyl) piperidin-1- yl)propanamido) butanoyl)-4- hydroxy-N-(4-(4- methylthiazol-5- yl)benzyl)pyrrolidine-2- carboxamide | (2S,4R)-1-((S)-3,3- dimethyl-2-(3-oxo-3- (4-(4-(4-(4-((E)-3- (pyridin-3- yl)acrylamido)butyl) piperidine-1- carbonyl)phenyl) piperidin-1- yl)propanamido) butanoyl)-4- hydroxy-N-(4-(4- methylthiazol-5- yl)benzyl)pyrrolidine- 2-carboxamide |
| | 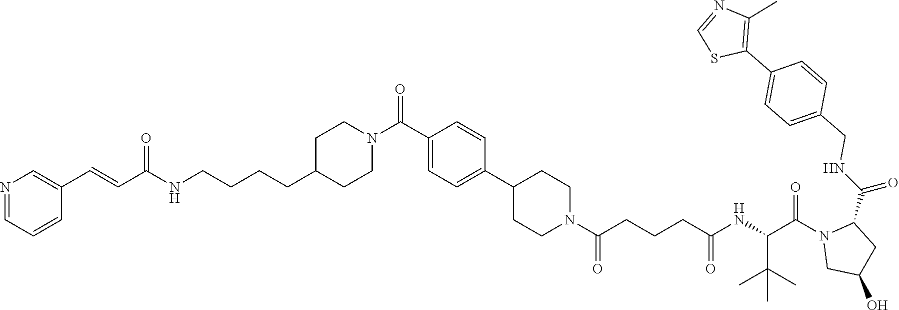 [SMILES: Cc1ncsc1-c1ccc(CNC(=O)[C@@H]2C[C@@H](O)CN2C(=O)[C@@H](NC(=O)CCCC(=O)N2CCC(c3ccc(C(=O)N4CCC(CCCCNC(=O)/C=C/c5cccnc5)CC4)cc3)CC2)C(C)(C)C)cc1] | (2S,4R)-1-((S)-3,3- dimethyl-2-(5-oxo-5-(4- (4-(4-(4-((E)-3- (pyridin-3- yl)acrylamido)butyl) piperidine-1- carbonyl)phenyl) piperidin-1- yl)pentanamido) butanoyl)-4-hydroxy- N-(4-(4- methylthiazol-5- yl)benzyl)pyrrolidine-2- carboxamide | (2S,4R)-1-((S)-3,3- dimethyl-2-(5-oxo-5- (4-(4-(4-(4-((E)-3- (pyridin-3- yl)acrylamido)butyl) piperidine-1- carbonyl)phenyl) piperidin-1- yl)pentanamido) butanoyl)-4- hydroxy-N-(4-(4- methylthiazol-5- yl)benzyl)pyrrolidine- 2-carboxamide |
| | 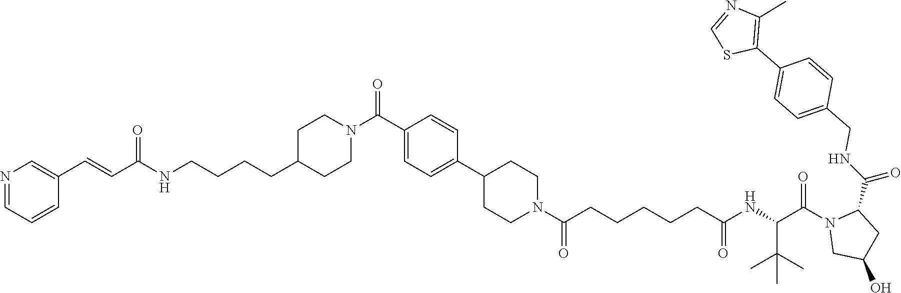 [SMILES: Cc1ncsc1-c1ccc(CNC(=O)[C@@H]2C[C@@H](O)CN2C(=O)[C@@H](NC(=O)CCCCCC(=O)N2CCC(c3ccc(C(=O)N4CCC(CCCCNC(=O)/C=C/c5cccnc5)CC4)cc3)CC2)C(C)(C)C)cc1] | (2S,4R)-1-((S)-3,3- dimethyl-2-(7-oxo-7-(4- (4-(4-(4-((E)-3- (pyridin-3- yl)acrylamido)butyl) piperidine-1- carbonyl)phenyl) piperidin-1- yl)heptanamido) butanoyl)-4- hydroxy-N-(4-(4- methylthiazol-5- yl)benzyl)pyrrolidine-2- carboxamide | (2S,4R)-1-((S)-3,3- dimethyl-2-(7-oxo-7- (4-(4-(4-(4-((E)-3- (pyridin-3- yl)acrylamido)butyl) piperidine-1- carbonyl)phenyl) piperidin-1- yl)heptanamido) butanoyl)-4- hydroxy-N-(4-(4- methylthiazol-5- yl)benzyl)pyrrolidine- 2-carboxamide |
| | 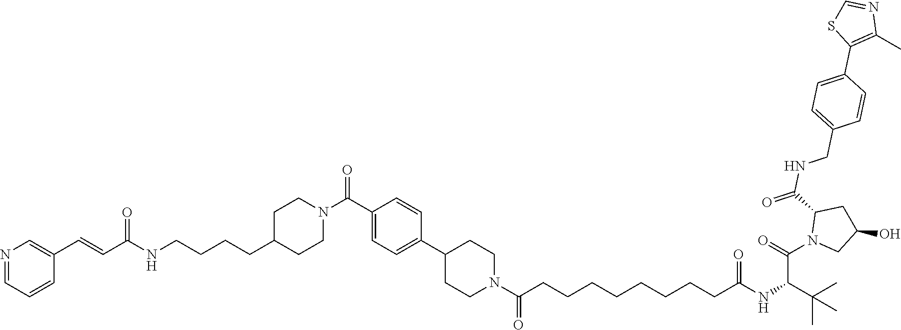 [SMILES: Cc1ncsc1-c1ccc(CNC(=O)[C@@H]2C[C@@H](O)CN2C(=O)[C@@H](NC(=O)CCCCCCCCC(=O)N2CCC(c3ccc(C(=O)N4CCC(CCCCNC(=O)/C=C/c5cccnc5)CC4)cc3)CC2)C(C)(C)C)cc1] | (2S,4R)-1-((S)-3,3- dimethyl-2-(10-oxo-10- (4-(4-(4-(4-((E)-3- (pyridin-3- yl)acrylamido)butyl) piperidine-1- carbonyl)phenyl) piperidin-1- yl)decanamido) butanoyl)-4- hydroxy-N-(4-(4- methylthiazol-5- yl)benzyl)pyrrolidine-2- carboxamide | (2S,4R)-1-((S)-3,3- dimethyl-2-(10-oxo-10- (4-(4-(4-(4-((E)-3- (pyridin-3- yl)acrylamido)butyl) piperidine-1- carbonyl)phenyl) piperidin-1- yl)decanamido) butanoyl)-4- hydroxy-N-(4-(4- methylthiazol-5- yl)benzyl)pyrrolidine- 2-carboxamide |
| | 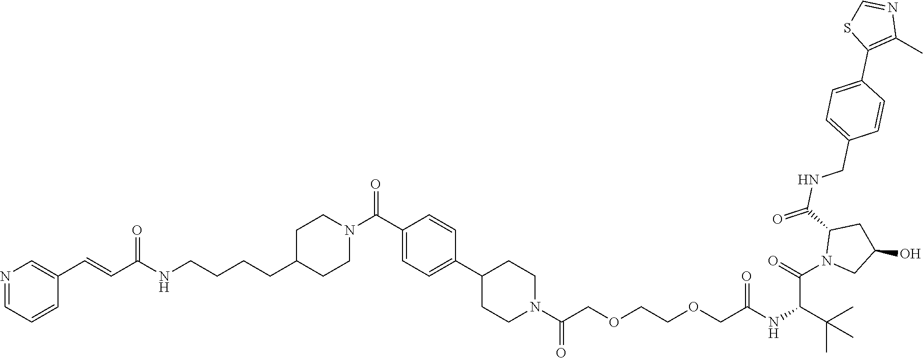 [SMILES: Cc1ncsc1-c1ccc(CNC(=O)[C@@H]2C[C@@H](O)CN2C(=O)[C@@H](NC(=O)COCCOCC(=O)N2CCC(c3ccc(C(=O)N4CCC(CCCCNC(=O)/C=C/c5cccnc5)CC4)cc3)CC2)C(C)(C)C)cc1] | (2S,4R)-1-((S)-3,3- dimethyl-2-(2-(2-(2- ox0-2-(4-(4-(4-(4-((E)- 3-(pyridin-3- yl)acrylamido)butyl) piperidine-1- carbonyl)phenyl) piperidin-1- yl)ethoxy)ethoxy) acetamido)butanoyl)- 4-hydroxy-N-(4-(4- methylthiazol-5- yl)benzyl)pyrrolidine-2- carboxamide | (2S,4R)-1-((S)-3,3- dimethyl-2-(2-(2-(2- oxo-2-(4-(4-(4-(4-((E)- 3-(pyridin-3- yl)acrylamido)butyl) piperidine-1- carbonyl)phenyl)piperi din-1- yl)ethoxy)ethoxy) acetamido)butanoyl)-4- hydroxy-N-(4-(4- methylthiazol-5- yl)benzyl)pyrrolidine- 2-carboxamide |
| | 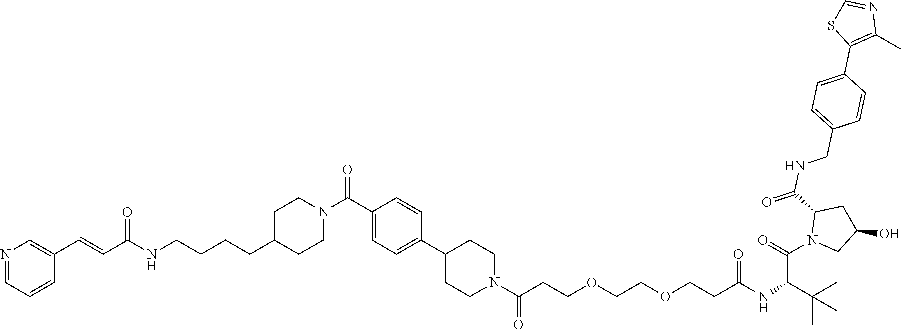 [SMILES: Cc1ncsc1-c1ccc(CNC(=O)[C@@H]2C[C@@H](O)CN2C(=O)[C@@H](NC(=O)CCOCCOCCC(=O)N2CCC(c3ccc(C(=O)N4CCC(CCCCNC(=O)/C=C/c5cccnc5)CC4)cc3)CC2)C(C)(C)C)cc1] | (2S,4R)-1-((S)-3,3- dimethyl-2-(3-(2-(3- ox0-3-(4-(4-(4-(4-((E)- 3-(pyridin-3- yl)acrylamido)butyl) piperidine-1- carbonyl)phenyl) piperidin-1- yl)propoxy)ethoxy) propanamido) butanoyl)-4- hydroxy-N-(4-(4- methylthiazol-5- yl)benzyl)pyrrolidine-2- carboxamide | (2S,4R)-1-((S)-3,3- dimethyl-2-(3-(2-(3- oxo-3-(4-(4-(4-(4-((E)- 3-(pyridin-3- yl)acrylamido)butyl) piperidine-1- carbonyl)phenyl) piperidin-1- yl)propoxy)ethoxy) propanamido) butanoyl)-4- hydroxy-N-(4-(4- methylthiazol-5- yl)benzyl)pyrrolidine- 2-carboxamide |
| | 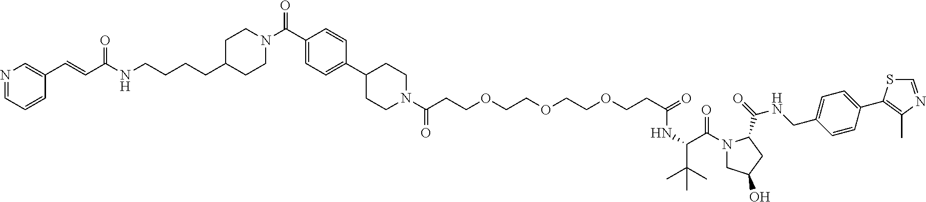 [SMILES: Cc1ncsc1-c1ccc(CNC(=O)[C@@H]2C[C@@H](O)CN2C(=O)[C@@H](NC(=O)CCOCCOCCOCCC(=O)N2CCC(c3ccc(C(=O)N4CCC(CCCCNC(=O)/C=C/c5cccnc5)CC4)cc3)CC2)C(C)(C)C)cc1] | (2S,4R)-1-((S)-2-(tert- butyl)-4, 16-dioxo-16-(4- (4-(4-(4-((E)-3-(pyridin- 3-yl)acrylamido) butyl)piperidine-1- carbonyl)phenyl) piperidin-1-yl)- 7,10,13-trioxa- 3-azahexadecanoyl)-4- hydroxy-N-(4-(4- methylthiazol-5- yl)benzyl)pyrrolidine-2- carboxamide | (2S,4R)-1-((S)-2-(tert- butyl)-4,16-dioxo-16- (4-(4-(4-(4-((E)-3- (pyridin-3- yl)acrylamido)butyl) piperidine-1- carbonyl)phenyl) piperidin-1-yl)- 7,10,13-trioxa-3- azahexadecanoyl)-4- hydroxy-N-(4-(4- methylthiazol-5- yl)benzyl)pyrrolidine- 2-carboxamide |
| | 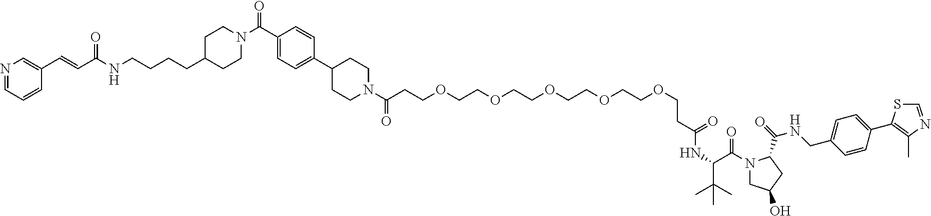 [SMILES: Cc1ncsc1-c1ccc(CNC(=O)[C@@H]2C[C@@H](O)CN2C(=O)[C@@H](NC(=O)CCOCCOCCOCCOCCOCCC(=O)N2CCC(c3ccc(C(=O)N4CCC(CCCCNC(=O)/C=C/c5cccnc5)CC4)cc3)CC2)C(C)(C)C)cc1] | (2S,4R)-1-((S)-2-(tert- butyl)-4,22-dioxo-22-(4- (4-(4-(4-((E)-3- (pyridin-3- yl)acrylamido)butyl) piperidine-1- carbonyl)phenyl) piperidin-1-yl)- 7,10,13,16,19- pentaoxa-3- azadocosanoyl)-4- hydroxy-N-(4-(4- methylthiazol-5- yl)benzyl)pyrrolidine-2- carboxamide | (2S,4R)-1-((S)-2-(tert- butyl)-4,22-dioxo-22- (4-(4-(4-(4-((E)-3- (pyridin-3- yl)acrylamido)butyl) piperidine-1- carbonyl)phenyl) piperidin-1-yl)- 7,10,13,16,19- pentaoxa-3- azadocosanoyl)-4- hydroxy-N-(4-(4- methylthiazol-5- yl)benzyl)pyrrolidine- 2-carboxamide |
| | 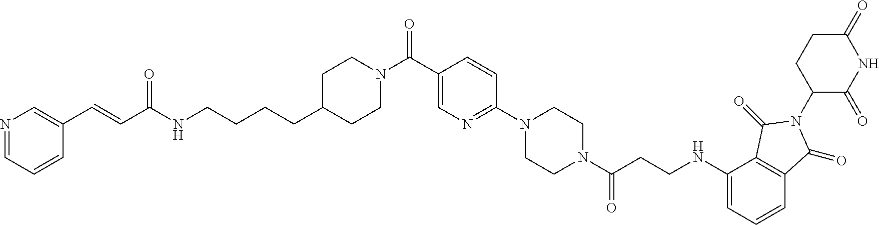 [SMILES: O=C(/C=C/c1cccnc1)NCCCCC1CCN(C(=O)c2ccc(N3CCN(C(=O)CCNc4cccc5c4C(=O)N(C4CCC(=O)NC4=O)C5=O)CC3)nc2)CC1] | (E)-N-(4-(1-(6-(4-(3- ((2-(2,6-dioxopiperidin- 3-yl)-1,3- dioxoisoindolin-4- yl)amino)propanoyl) piperazin-1- yl)nicotinoyl)piperidin- 4-yl)butyl)-3-(pyridin-3- yl)acrylamide | (E)-N-(4-(1-(6-(4-(3- ((2-(2,6- dioxopiperidin-3-yl)- 1,3-dioxoisoindolin-4- yl)amino)propanoyl) piperazin-1- yl)nicotinoyl)piperidin- 4-yl)butyl)-3-(pyridin- 3-yl)acrylamide |
| | 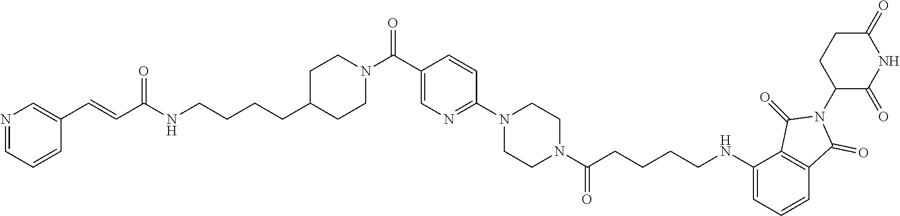 [SMILES: O=C(/C=C/c1cccnc1)NCCCCC1CCN(C(=O)c2ccc(N3CCN(C(=O)CCCCNc4cccc5c4C(=O)N(C4CCC(=O)NC4=O)C5=O)CC3)nc2)CC1] | (E)-N-(4-(1-(6-(4-(5- ((2-(2,6-dioxopiperidin- 3-yl)-1,3- dioxoisoindolin-4- yl)amino)pentanoyl) piperazin-1- yl)nicotinoyl)piperidin- 4-yl)butyl)-3-(pyridin-3- yl)acrylamide | (E)-N-(4-(1-(6-(4-(5- ((2-(2,6- dioxopiperidin-3-yl)- 1,3-dioxoisoindolin-4- yl)amino)pentanoyl) piperazin-1- yl)nicotinoyl)piperidin- 4-yl)butyl)-3-(pyridin- 3-yl)acrylamide |
| | 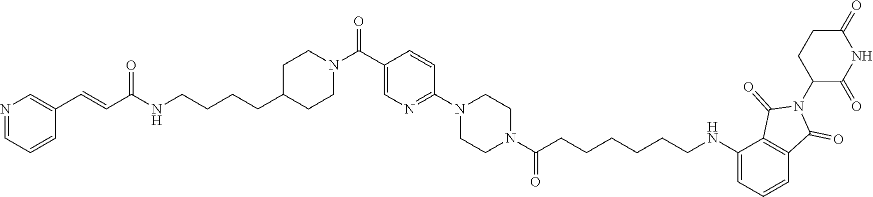 [SMILES: O=C(/C=C/c1cccnc1)NCCCCC1CCN(C(=O)c2ccc(N3CCN(C(=O)CCCCCCNc4cccc5c4C(=O)N(C4CCC(=O)NC4=O)C5=O)CC3)nc2)CC1] | (E)-N-(4-(1-(6-(4-(7- ((2-(2,6-dioxopiperidin- 3-yl)-1,3- dioxoisoindolin-4- yl)amino)heptanoyl) piperazin-1- yl)nicotinoyl)piperidin- 4-yl)butyl)-3-(pyridin-3- yl)acrylamide | (E)-N-(4-(1-(6-(4-(7- ((2-(2,6- dioxopiperidin-3-yl)- 1,3-dioxoisoindolin-4- yl)amino)heptanoyl) piperazin-1- yl)nicotinoyl)piperidin- 4-yl)butyl)-3-(pyridin- 3-yl)acrylamide |
| | 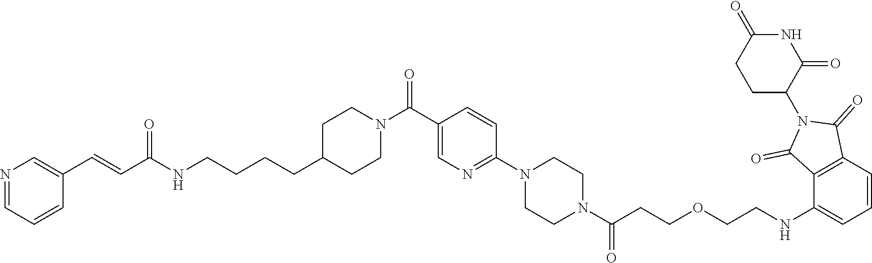 [SMILES: O=C(/C=C/c1cccnc1)NCCCCC1CCN(C(=O)c2ccc(N3CCN(C(=O)CCOCCNc4cccc5c4C(=O)N(C4CCC(=O)NC4=O)C5=O)CC3)nc2)CC1] | (E)-N-(4-(1-(6-(4-(3-(2- ((2-(2,6-dioxopiperidin- 3-yl)-1,3- dioxoisoindolin-4- yl)amino)ethoxy) propanoyl)piperazin-1- yl)nicotinoyl)piperidin- 4-yl)butyl)-3-(pyridin-3- yl)acrylamide | (E)-N-(4-(1-(6-(4-(3- (2-((2-(2,6- dioxopiperidin-3-yl)- 1,3-dioxoisoindolin-4- yl)amino)ethoxy) propanoyl)piperazin-1- yl)nicotinoyl)piperidin- 4-yl)butyl)-3-(pyridin- 3-yl)acrylamide |
| | 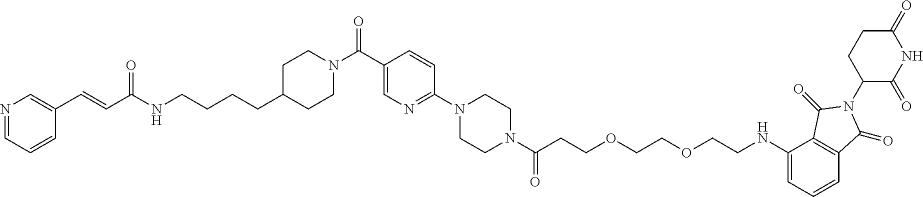 [SMILES: O=C(/C=C/c1cccnc1)NCCCCC1CCN(C(=O)c2ccc(N3CCN(C(=O)CCOCCOCCNc4cccc5c4C(=O)N(C4CCC(=O)NC4=O)C5=O)CC3)nc2)CC1] | (E)-N-(4-(1-(6-(4-(3-(2- (2-((2-(2,6- dioxopiperidin-3-yl)- 1,3-dioxoisoindolin-4- yl)amino)ethoxy)ethoxy) propanoyl)piperazin-1- yl)nicotinoyl)piperidin- 4-yl)butyl)-3-(pyridin-3- yl)acrylamide | (E)-N-(4-(1-(6-(4-(3- (2-(2-((2-(2,6- dioxopiperidin-3-yl)- 1,3-dioxoisoindolin-4- yl)amino)ethoxy) ethoxy)propanoyl) piperazin-1- yl)nicotinoyl)piperidin- 4-yl)butyl)-3-(pyridin- 3-yl)acrylamide |
| | 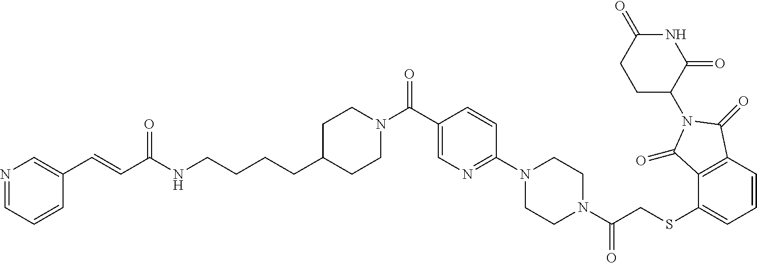 [SMILES: O=C(/C=C/c1cccnc1)NCCCCC1CCN(C(=O)c2ccc(N3CCN(C(=O)CSc4cccc5c4C(=O)N(C4CCC(=O)NC4=O)C5=O)CC3)nc2)CC1] | (E)-N-(4-(1-(6-(4-(2- ((2-(2,6-dioxopiperidin- 3-yl)-1,3- dioxoisoindolin-4- yl)thio)acetyl) piperazin-1- yl)nicotinoyl)piperidin- 4-yl)butyl)-3-(pyridin-3- yl)acrylamide | (E)-N-(4-(1-(6-(4-(2- ((2-(2,6- dioxopiperidin-3-yl)- 1,3-dioxoisoindolin-4- yl)thio)acetyl) piperazin-1- yl)nicotinoyl)piperidin- 4-yl)butyl)-3-(pyridin- 3-yl)acrylamide |
| | 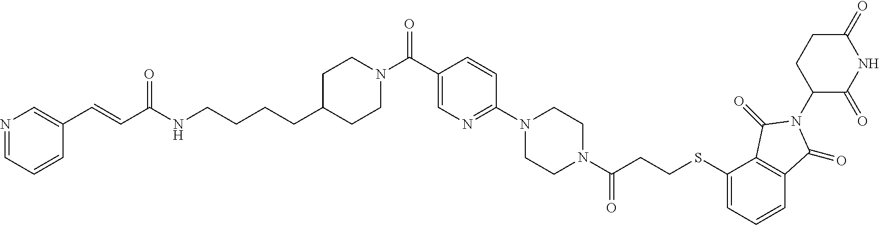 [SMILES: O=C(/C=C/c1cccnc1)NCCCCC1CCN(C(=O)c2ccc(N3CCN(C(=O)CCSc4cccc5c4C(=O)N(C4CCC(=O)NC4=O)C5=O)CC3)nc2)CC1] | (E)-N-(4-(1-(6-(4-(3- ((2-(2,6-dioxopiperidin- 3-yl)-1,3- dioxoisoindolin-4- yl)thio)propanoyl) piperazin-1- yl)nicotinoyl)piperidin- 4-yl)butyl)-3-(pyridin-3- yl)acrylamide | (E)-N-(4-(1-(6-(4-(3- ((2-(2,6- dioxopiperidin-3-yl)- 1,3-dioxoisoindolin-4- yl)thio)propanoyl) piperazin-1- yl)nicotinoyl)piperidin- 4-yl)butyl)-3-(pyridin- 3-yl)acrylamide |
| | 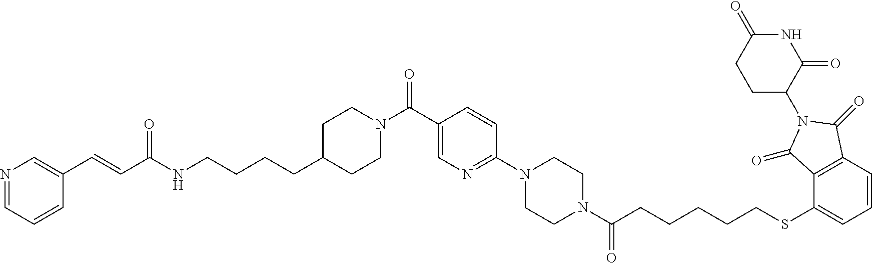 [SMILES: O=C(/C=C/c1cccnc1)NCCCCC1CCN(C(=O)c2ccc(N3CCN(C(=O)CCCCCSc4cccc5c4C(=O)N(C4CCC(=O)NC4=O)C5=O)CC3)nc2)CC1] | (E)-N-(4-(1-(6-(4-(6- ((2-(2,6-dioxopiperidin- 3-yl)-1,3- dioxoisoindolin-4- yl)thio)hexanoyl) piperazin-1- yl)nicotinoyl)piperidin- 4-yl)butyl)-3-(pyridin-3- yl)acrylamide | (E)-N-(4-(1-(6-(4-(6- ((2-(2,6- dioxopiperidin-3-yl)- 1,3-dioxoisoindolin-4- yl)thio)hexanoyl) piperazin-1- yl)nicotinoyl)piperidin- 4-yl)butyl)-3-(pyridin- 3-yl)acrylamide |
| | 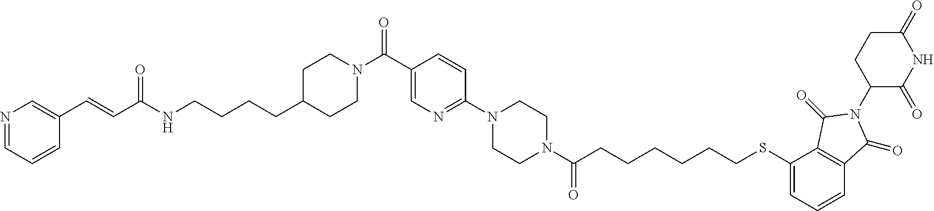 [SMILES: O=C(/C=C/c1cccnc1)NCCCCC1CCN(C(=O)c2ccc(N3CCN(C(=O)CCCCCCSc4cccc5c4C(=O)N(C4CCC(=O)NC4=O)C5=O)CC3)nc2)CC1] | (E)-N-(4-(1-(6-(4-(7- ((2-(2,6-dioxopiperidin- 3-yl)-1,3- dioxoisoindolin-4- yl)thio)heptanoyl) piperazin-1- yl)nicotinoyl)piperidin- 4-yl)butyl)-3-(pyridin-3- yl)acrylamide | (E)-N-(4-(1-(6-(4-(7- ((2-(2,6- dioxopiperidin-3-yl)- 1,3-dioxoisoindolin-4- yl)thio)heptanoyl) piperazin-1- yl)nicotinoyl)piperidin- 4-yl)butyl)-3-(pyridin- 3-yl)acrylamide |
| | 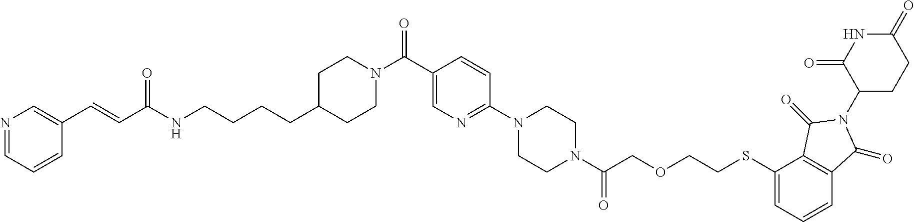 [SMILES: O=C(/C=C/c1cccnc1)NCCCCC1CCN(C(=O)c2ccc(N3CCN(C(=O)COCCSc4cccc5c4C(=O)N(C4CCC(=O)NC4=O)C5=O)CC3)nc2)CC1] | (E)-N-(4-(1-(6-(4-(2-(2- ((2-(2,6-dioxopiperidin- 3-yl)-1,3- dioxoisoindolin-4- yl)thio)ethoxy)acetyl) piperazin-1- yl)nicotinoyl)piperidin- 4-yl)butyl)-3-(pyridin-3- yl)acrylamide | (E)-N-(4-(1-(6-(4-(2- (2-((2-(2,6- dioxopiperidin-3-yl)- 1,3-dioxoisoindolin-4- yl)thio)ethoxy)acetyl) piperazin-1- yl)nicotinoyl)piperidin- 4-yl)butyl)-3-(pyridin- 3-yl)acrylamide |
| | 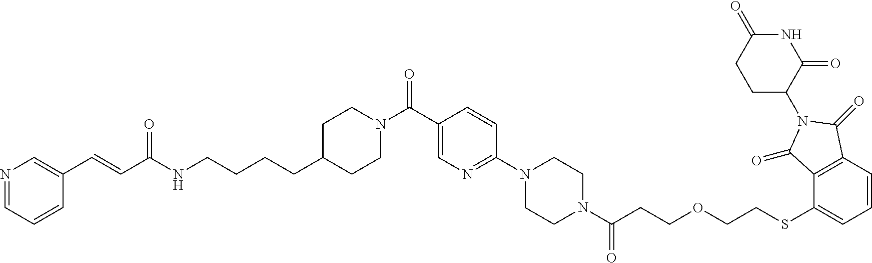 [SMILES: O=C(/C=C/c1cccnc1)NCCCCC1CCN(C(=O)c2ccc(N3CCN(C(=O)CCOCCSc4cccc5c4C(=O)N(C4CCC(=O)NC4=O)C5=O)CC3)nc2)CC1] | (E)-N-(4-(1-(6-(4-(3-(2- ((2-(2,6-dioxopiperidin- 3-yl)-1,3- dioxoisoindolin-4- yl)thio)ethoxy) propanoyl)piperazin-1- yl)nicotinoyl)piperidin- 4-yl)butyl)-3-(pyridin-3- yl)acrylamide | (E)-N-(4-(1-(6-(4-(3- (2-((2-(2,6- dioxopiperidin-3-yl)- 1,3-dioxoisoindolin-4- yl)thio)ethoxy)propano yl)piperazin-1- yl)nicotinoyl)piperidin- 4-yl)butyl)-3-(pyridin- 3-yl)acrylamide |
| | 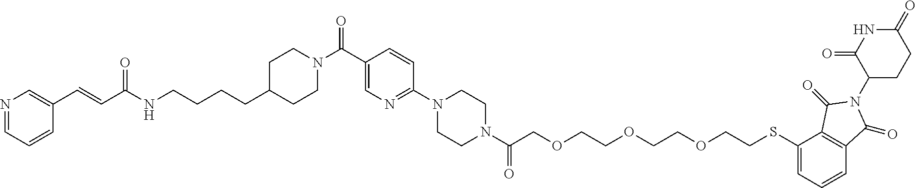 [SMILES: O=C(/C=C/c1cccnc1)NCCCCC1CCN(C(=O)c2ccc(N3CCN(C(=O)COCCOCCOCCSc4cccc5c4C(=O)N(C4CCC(=O)NC4=O)C5=O)CC3)nc2)CC1] | (E)-N-(4-(1-(6-(4-(2-(2- (2-(2-((2-(2,6- dioxopiperidin-3-yl)- 1,3-dioxoisoindolin-4- yl)thio)ethoxy)ethoxy) ethoxy)acetyl) piperazin-1- yl)nicotinoyl)piperidin- 4-yl)butyl)-3-(pyridin-3- yl)acrylamide | (E)-N-(4-(1-(6-(4-(2- (2-(2-(2-((2-(2,6- dioxopiperidin-3-yl)- 1,3-dioxoisoindolin-4- yl)thio)ethoxy)ethoxy) ethoxy)acetyl) piperazin-1- yl)nicotinoyl)piperidin- 4-yl)butyl)-3-(pyridin- 3-yl)acrylamide |
| | 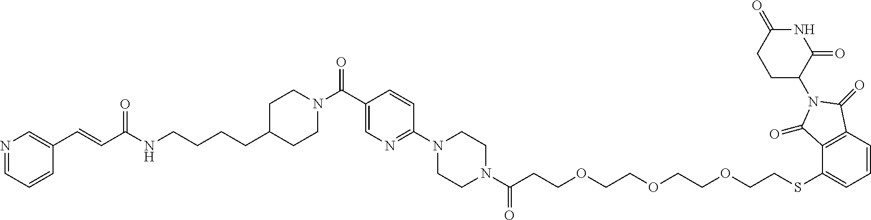 [SMILES: O=C(/C=C/c1cccnc1)NCCCCC1CCN(C(=O)c2ccc(N3CCN(C(=O)CCOCCOCCOCCSc4cccc5c4C(=O)N(C4CCC(=O)NC4=O)C5=O)CC3)nc2)CC1] | (E)-N-(4-(1-(6-(4-(3-(2- (2-(2-((2-(2,6- dioxopiperidin-3-yl)- 1,3-dioxoisoindolin-4- yl)thio)ethoxy)ethoxy) ethoxy)propanoyl) piperazin-1- yl)nicotinoyl)piperidin- 4-yl)butyl)-3-(pyridin-3- yl)acrylamide | (E)-N-(4-(1-(6-(4-(3- (2-(2-(2-((2-(2,6- dioxopiperidin-3-yl)- 1,3-dioxoisoindolin-4- yl)thio)ethoxy)ethoxy) ethoxy)propanoyl) piperazin-1- yl)nicotinoyl)piperidin- 4-yl)butyl)-3-(pyridin- 3-yl)acrylamide |
| | 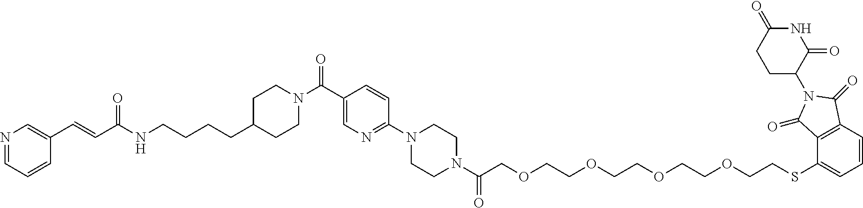 [SMILES: O=C(/C=C/c1cccnc1)NCCCCC1CCN(C(=O)c2ccc(N3CCN(C(=O)COCCOCCOCCOCCSc4cccc5c4C(=O)N(C4CCC(=O)NC4=O)C5=O)CC3)nc2)CC1] | (E)-N-(4-(1-(6-(4-(14- ((2-(2,6-dioxopiperidin- 3-yl)-1,3- dioxoisoindolin-4- yl)thio)-3,6,9,12- tetraoxatetradecanoyl) piperazin-1- yl)nicotinoyl)piperidin- 4-yl)butyl)-3-(pyridin-3- yl)acrylamide | (E)-N-(4-(1-(6-(4-(14- ((2-(2,6- dioxopiperidin-3-yl)- 1,3-dioxoisoindolin-4- yl)thio)-3,6,9,12- tetraoxatetradecanoyl) piperazin-1- yl)nicotinoyl)piperidin- 4-yl)butyl)-3-(pyridin- 3-yl)acrylamide |
| | 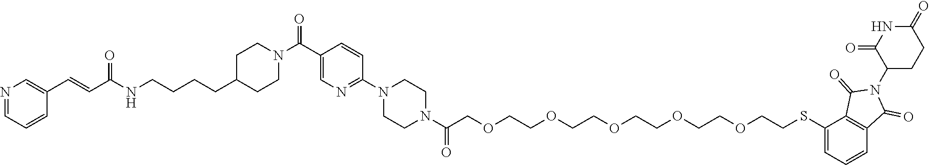 [SMILES: O=C(/C=C/c1cccnc1)NCCCCC1CCN(C(=O)c2ccc(N3CCN(C(=O)COCCOCCOCCOCCOCCSc4cccc5c4C(=O)N(C4CCC(=O)NC4=O)C5=O)CC3)nc2)CC1] | (E)-N-(4-(1-(6-(4-(17- ((2-(2,6-dioxopiperidin- 3-yl)-1,3- dioxoisoindolin-4- yl)thio)-3,6,9,12,15- pentaoxaheptadecanoyl) piperazin-1- yl)nicotinoyl)piperidin- 4-yl)butyl)-3-(pyridin-3- yl)acrylamide | (E)-N-(4-(1-(6-(4-(17- ((2-(2,6- dioxopiperidin-3-yl)- 1,3-dioxoisoindolin-4- yl)thio)-3,6,9,12,15- pentaoxahepta- decanoyl)piperazin-1- yl)nicotinoyl)piperidin- 4-yl)butyl)-3-(pyridin- 3-yl)acrylamide |
| | 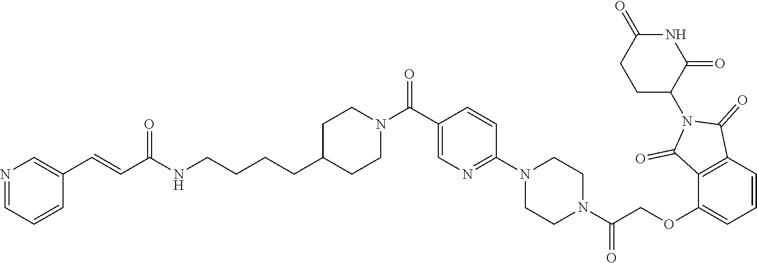 [SMILES: O=C(/C=C/c1cccnc1)NCCCCC1CCN(C(=O)c2ccc(N3CCN(C(=O)COc4cccc5c4C(=O)N(C4CCC(=O)NC4=O)C5=O)CC3)nc2)CC1] | (E)-N-(4-(1-(6-(4-(2- ((2-(2,6-dioxopiperidin- 3-yl)-1,3- dioxoisoindolin-4- yl)oxy)acetyl) piperazin-1- yl)nicotinoyl)piperidin- 4-yl)butyl)-3-(pyridin-3- yl)acrylamide | (E)-N-(4-(1-(6-(4-(2- ((2-(2,6- dioxopiperidin-3-yl)- 1,3-dioxoisoindolin-4- yl)oxy)acetyl) piperazin-1- yl)nicotinoyl)piperidin- 4-yl)butyl)-3-(pyridin- 3-yl)acrylamide |
| | 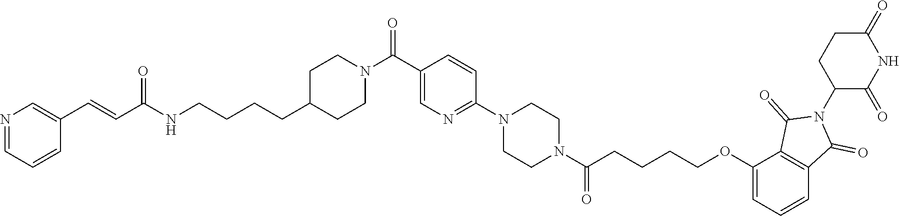 [SMILES: O=C(/C=C/c1cccnc1)NCCCCC1CCN(C(=O)c2ccc(N3CCN(C(=O)CCCCOc4cccc5c4C(=O)N(C4CCC(=O)NC4=O)C5=O)CC3)nc2)CC1] | (E)-N-(4-(1-(6-(4-(5- ((2-(2,6-dioxopiperidin- 3-yl)-1,3- dioxoisoindolin-4- yl)oxy)pentanoyl) piperazin-1- yl)nicotinoyl)piperidin- 4-yl)butyl)-3-(pyridin-3- yl)acrylamide | (E)-N-(4-(1-(6-(4-(5- ((2-(2,6- dioxopiperidin-3-yl)- 1,3-dioxoisoindolin-4- yl)oxy)pentanoyl) piperazin-1- yl)nicotinoyl)piperidin- 4-yl)butyl)-3-(pyridin- 3-yl)acrylamide |
| | 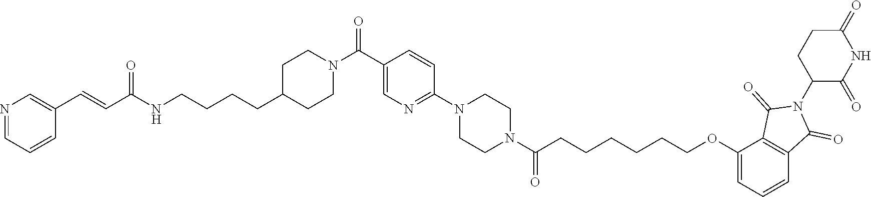 [SMILES: O=C(/C=C/c1cccnc1)NCCCCC1CCN(C(=O)c2ccc(N3CCN(C(=O)CCCCCCOc4cccc5c4C(=O)N(C4CCC(=O)NC4=O)C5=O)CC3)nc2)CC1] | (E)-N-(4-(1-(6-(4-(7- ((2-(2,6-dioxopiperidin- 3-yl)-1,3- dioxoisoindolin-4- yl)oxy)heptanoyl) piperazin-1- yl)nicotinoyl)piperidin- 4-yl)butyl)-3-(pyridin-3- yl)acrylamide | (E)-N-(4-(1-(6-(4-(7- ((2-(2,6- dioxopiperidin-3-yl)- 1,3-dioxoisoindolin-4- yl)oxy)heptanoyl) piperazin-1 - yl)nicotinoyl)piperidin- 4-yl)butyl)-3-(pyridin- 3-yl)acrylamide |
| | 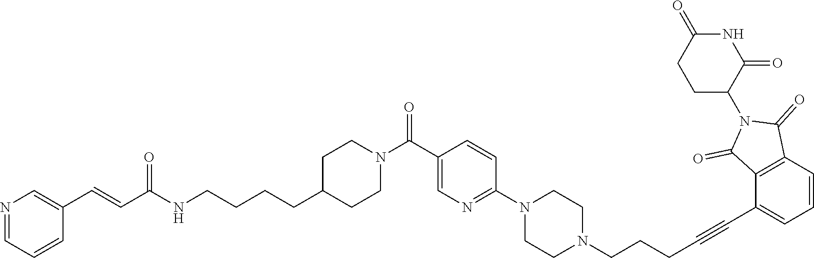 [SMILES: O=C(/C=C/c1cccnc1)NCCCCC1CCN(C(=O)c2ccc(N3CCN(CCCC#Cc4cccc5c4C(=O)N(C4CCC(=O)NC4=O)C5=O)CC3)nc2)CC1] | (E)-N-(4-(1-(6-(4-(5-(2- (2,6-dioxopiperidin-3- yl)-1,3-dioxoisoindolin- 4-yl)pent-4-yn-1- yl)piperazin-1- yl)nicotinoyl)piperidin- 4-yl)butyl)-3-(pyridin-3- yl)acrylamide | (E)-N-(4-(1-(6-(4-(5- (2-(2,6-dioxopiperidin- 3-yl)-1,3- dioxoisoindolin-4- yl)pent-4-yn-1- yl)piperazin-1- yl)nicotinoyl)piperidin- 4-yl)butyl)-3-(pyridin- 3-yl)acrylamide |
| | 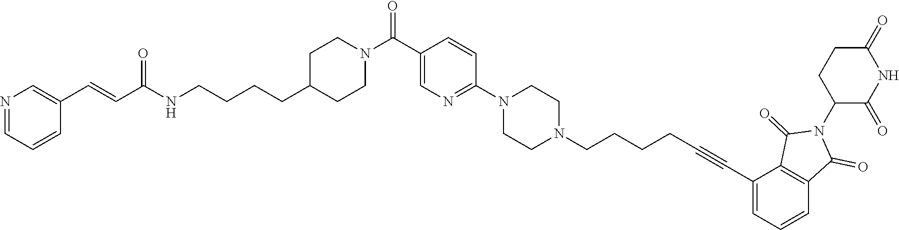 [SMILES: O=C(/C=C/c1cccnc1)NCCCCC1CCN(C(=O)c2ccc(N3CCN(CCCCC#Cc4cccc5c4C(=O)N(C4CCC(=O)NC4=O)C5=O)CC3)nc2)CC1] | (E)-N-(4-(1-(6-(4-(6-(2- (2,6-dioxopiperidin-3- yl)-1,3-dioxoisoindolin- 4-yl)hex-5-yn-1- yl)piperazin-1- yl)nicotinoyl)piperidin- 4-yl)butyl)-3-(pyridin-3- yl)acrylamide | (E)-N-(4-(1-(6-(4-(6- (2-(2,6-dioxopiperidin- 3-yl)-1,3- dioxoisoindolin-4- yl)hex-5-yn-1- yl)piperazin-1- yl)nicotinoyl)piperidin- 4-yl)butyl)-3-(pyridin- 3-yl)acrylamide |
| | 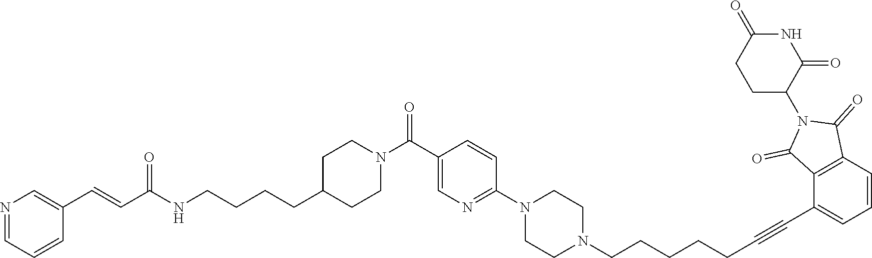 [SMILES: O=C(/C=C/c1cccnc1)NCCCCC1CCN(C(=O)c2ccc(N3CCN(CCCCCC#Cc4cccc5c4C(=O)N(C4CCC(=O)NC4=O)C5=O)CC3)nc2)CC1] | (E)-N-(4-(1-(6-(4-(7-(2- (2,6-dioxopiperidin-3- yl)-1,3-dioxoisoindolin- 4-yl)hept-6-yn-1- yl)piperazin-1- yl)nicotinoyl)piperidin- 4-yl)butyl)-3-(pyridin-3- yl)acrylamide | (E)-N-(4-(1-(6-(4-(7- (2-(2,6-dioxopiperidin- 3-yl)-1,3- dioxoisoindolin-4- yl)hept-6-yn-1- yl)piperazin-1- yl)nicotinoyl)piperidin- 4-yl)butyl)-3-(pyridin- 3-yl)acrylamide |
| | 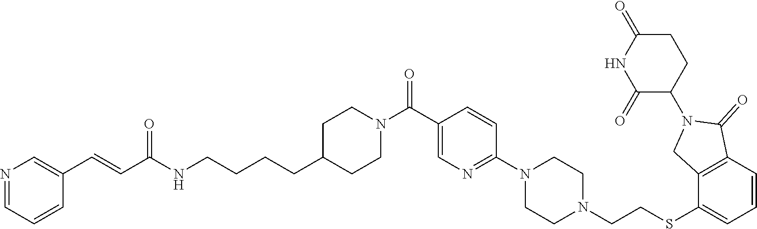 [SMILES: O=C(/C=C/c1cccnc1)NCCCCC1CCN(C(=O)c2ccc(N3CCN(CCSc4cccc5c4CN(C4CCC(=O)NC4=O)C5=O)CC3)nc2)CC1] | (E)-N-(4-(1-(6-(4-(2- ((2-(2,6-dioxopiperidin- 3-yl)-1,3- dioxoisoindolin-4- yl)thio)ethyl) piperazin-1- yl)nicotinoyl)piperidin- 4-yl)butyl)-3-(pyridin-3- yl)acrylamide | (E)-N-(4-(1-(6-(4-(2- ((2-(2,6- dioxopiperidin-3-yl)- 1,3-dioxoisoindolin-4- yl)thio)ethyl) piperazin-1- yl)nicotinoyl)piperidin- 4-yl)butyl)-3-(pyridin- 3-yl)acrylamide |
| | 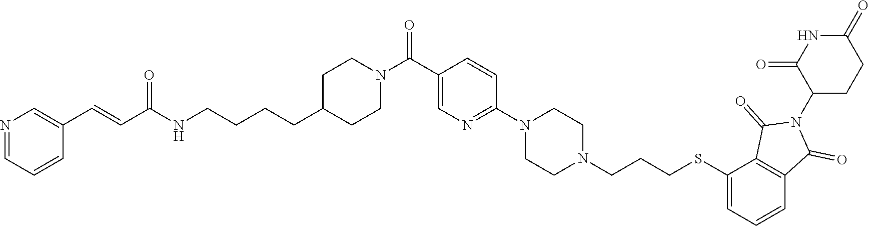 [SMILES: O=C(/C=C/c1cccnc1)NCCCCC1CCN(C(=O)c2ccc(N3CCN(CCCSc4cccc5c4C(=O)N(C4CCC(=O)NC4=O)C5=O)CC3)nc2)CC1] | (E)-N-(4-(1-(6-(4-(3- ((2-(2,6-dioxopiperidin- 3-yl)-1,3- dioxoisoindolin-4- yl)thio)propyl) piperazin-1- yl)nicotinoyl)piperidin- 4-yl)butyl)-3-(pyridin-3- yl)acrylamide | (E)-N-(4-(1-(6-(4-(3- ((2-(2,6- dioxopiperidin-3-yl)- 1,3-dioxoisoindolin-4- yl)thio)propyl) piperazin-1- yl)nicotinoyl)piperidin- 4-yl)butyl)-3-(pyridin- 3-yl)acrylamide |
| | 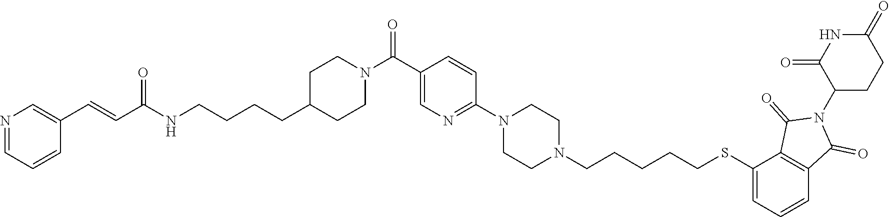 [SMILES: O=C(/C=C/c1cccnc1)NCCCCC1CCN(C(=O)c2ccc(N3CCN(CCCCCSc4cccc5c4C(=O)N(C4CCC(=O)NC4=O)C5=O)CC3)nc2)CC1] | (E)-N-(4-(1-(6-(4-(5- ((2-(2,6-dioxopiperidin- 3-yl)-1,3- dioxoisoindolin-4- yl)thio)pentyl)p iperazin-1- yl)nicotinoyl)piperidin- 4-yl)butyl)-3-(pyridin-3- yl)acrylamide | (E)-N-(4-(1-(6-(4-(5- ((2-(2,6- dioxopiperidin-3-yl)- 1,3-dioxoisoindolin-4- yl)thio)pentyl) piperazin-1- yl)nicotinoyl)piperidin- 4-yl)butyl)-3-(pyridin- 3-yl)acrylamide |
| | 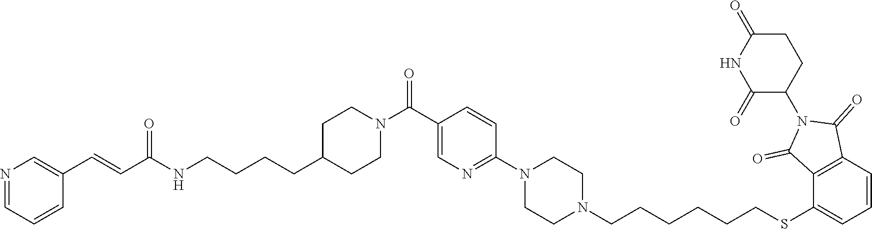 [SMILES: O=C(/C=C/c1cccnc1)NCCCCC1CCN(C(=O)c2ccc(N3CCN(CCCCCCSc4cccc5c4C(=O)N(C4CCC(=O)NC4=O)C5=O)CC3)nc2)CC1] | (E)-N-(4-(1-(6-(4-(6- ((2-(2,6-dioxopiperidin- 3-yl)-1,3- dioxoisoindolin-4- yl)thio)hexyl) piperazin-1- yl)nicotinoyl)piperidin- 4-yl)butyl)-3-(pyridin-3- yl)acrylamide | (E)-N-(4-(1-(6-(4-(6- ((2-(2,6- dioxopiperidin-3-yl)- 1,3-dioxoisoindolin-4- yl)thio)hexyl) piperazin-1- yl)nicotinoyl)piperidin- 4-yl)butyl)-3-(pyridin- 3-yl)acrylamide |
| | 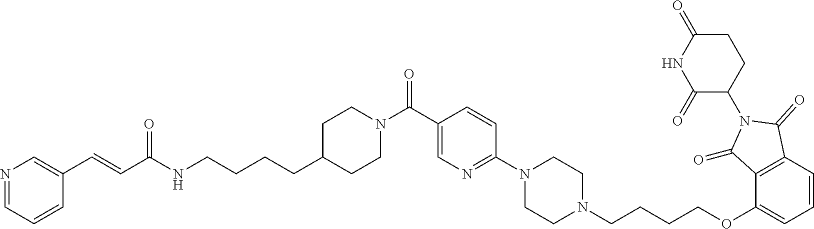 [SMILES: O=C(/C=C/c1cccnc1)NCCCCC1CCN(C(=O)c2ccc(N3CCN(CCCCOc4cccc5c4C(=O)N(C4CCC(=O)NC4=O)C5=O)CC3)nc2)CC1] | (E)-N-(4-(1-(6-(4-(4- ((2-(2,6-dioxopiperidin- 3-yl)-1,3- dioxoisoindolin-4- yl)oxy)butyl) piperazin-1- yl)nicotinoyl)piperidin- 4-yl)butyl)-3-(pyridin-3- yl)acrylamide | (E)-N-(4-(1-(6-(4-(4- ((2-(2,6- dioxopiperidin-3-yl)- 1,3-dioxoisoindolin-4- yl)oxy)butyl) piperazin-1- yl)nicotinoyl)piperidin- 4-yl)butyl)-3-(pyridin- 3-yl)acrylamide |
| | 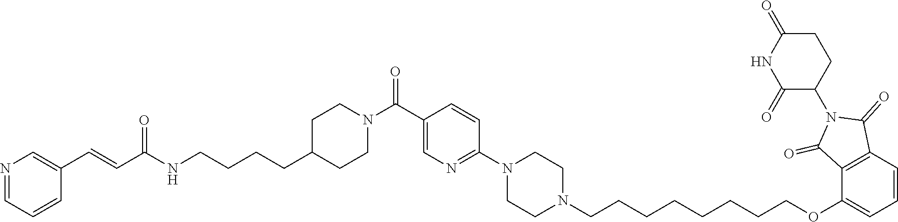 [SMILES: O=C(/C=C/c1cccnc1)NCCCCC1CCN(C(=O)c2ccc(N3CCN(CCCCCCCCOc4cccc5c4C(=O)N(C4CCC(=O)NC4=O)C5=O)CC3)nc2)CC1] | (E)-N-(4-(1-(6-(4-(8- ((2-(2,6-dioxopiperidin- 3-yl)-1,3- dioxoisoindolin-4- yl)oxy)octyl) piperazin-1- yl)nicotinoyl)piperidin- 4-yl)butyl)-3-(pyridin-3- yl)acrylamide | (E)-N-(4-(1-(6-(4-(8- ((2-(2,6- dioxopiperidin-3-yl)- 1,3-dioxoisoindolin-4- yl)oxy)octyl) piperazin-1- yl)nicotinoyl)piperidin- 4-yl)butyl)-3-(pyridin- 3-yl)acrylamide |
| | 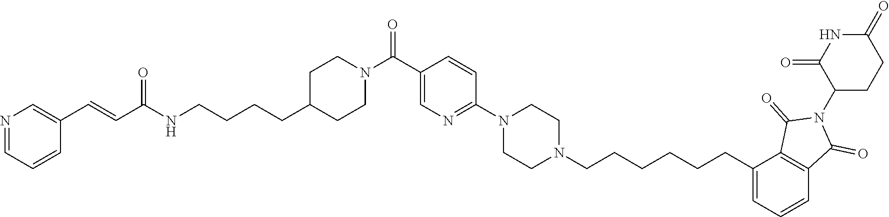 [SMILES: O=C(/C=C/c1cccnc1)NCCCCC1CCN(C(=O)c2ccc(N3CCN(CCCCCCc4cccc5c4C(=O)N(C4CCC(=O)NC4=O)C5=O)CC3)nc2)CC1] | (E)-N-(4-(1-(6-(4-(6-(2- (2,6-dioxopiperidin-3- yl)-1,3-dioxoisoindolin- 4-yl)hexyl)piperazin-1- yl)nicotinoyl)piperidin- 4-yl)butyl)-3-(pyridin-3- yl)acrylamide | (E)-N-(4-(1-(6-(4-(6- (2-(2,6-dioxopiperidin- 3-yl)-1,3- dioxoisoindolin-4- yl)hexyl)piperazin-1- yl)nicotinoyl)piperidin- 4-yl)butyl)-3-(pyridin- 3-yl)acrylamide |
| | 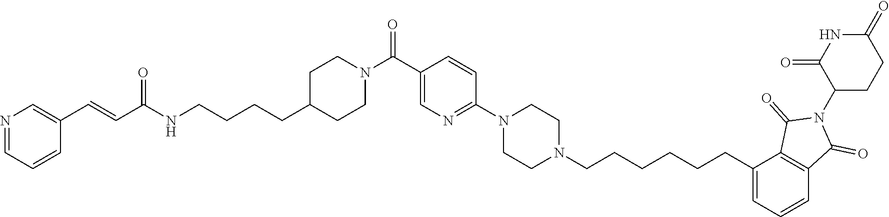 [SMILES: O=C(/C=C/c1cccnc1)NCCCCC1CCN(C(=O)c2ccc(N3CCN(CCCCCCc4cccc5c4C(=O)N(C4CCC(=O)NC4=O)C5=O)CC3)nc2)CC1] | (E)-N-(4-(1-(6-(4-(7-(2- (2,6-dioxopiperidin-3- yl)-1,3-dioxoisoindolin- 4-yl)heptyl)piperazin-1- yl)nicotinoyl)piperidin- 4-yl)butyl)-3-(pyridin-3- yl)acrylamide | (E)-N-(4-(1-(6-(4-(7- (2-(2,6-dioxopiperidin- 3-yl)-1,3- dioxoisoindolin-4- yl)heptyl)piperazin-1- yl)nicotinoyl)piperidin- 4-yl)butyl)-3-(pyridin- 3-yl)acrylamide |
| | 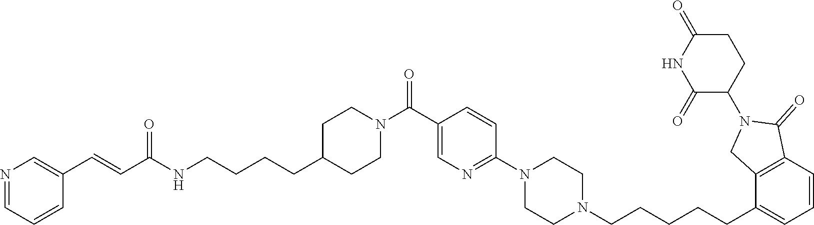 [SMILES: O=C(/C=C/c1cccnc1)NCCCCC1CCN(C(=O)c2ccc(N3CCN(CCCCCc4cccc5c4CN(C4CCC(=O)NC4=O)C5=O)CC3)nc2)CC1] | (E)-N-(4-(1-(6-(4-(5-(2- (2,6-dioxopiperidin-3- yl)-1,3-dioxoisoindolin- 4-yl)pentyl)piperazin-1- yl)nicotinoyl)piperidin- 4-yl)butyl)-3-(pyridin-3- yl)acrylamide | (E)-N-(4-(1-(6-(4-(5- (2-(2,6-dioxopiperidin- 3-yl)-1,3- dioxoisoindolin-4- yl)pentyl)piperazin-1- yl)nicotinoyl)piperidin- 4-yl)butyl)-3-(pyridin- 3-yl)acrylamide |
| | 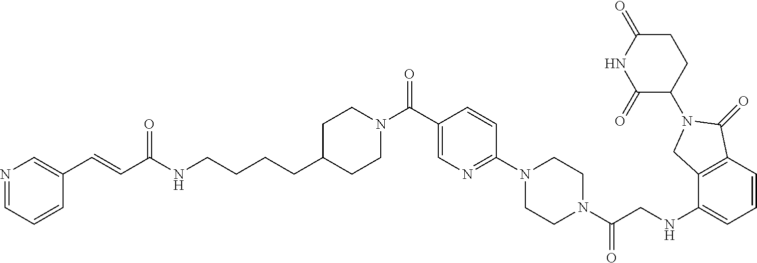 [SMILES: O=C(/C=C/c1cccnc1)NCCCCC1CCN(C(=O)c2ccc(N3CCN(C(=O)CNc4cccc5c4CN(C4CCC(=O)NC4=O)C5=O)CC3)nc2)CC1] | (E)-N-(4-(1-(6-(4-((2- (2,6-dioxopiperidin-3- yl)-1-oxoisoindolin-4- yl)glycyl)piperazin-1- yl)nicotinoyl)piperidin- 4-yl)butyl)-3-(pyridin-3- yl)acrylamide | (E)-N-(4-(1-(6-(4-((2- (2,6-dioxopiperidin-3- yl)-1-oxoisoindolin-4- yl)glycyl)piperazin-1- yl)nicotinoyl)piperidin- 4-yl)butyl)-3-(pyridin- 3-yl)acrylamide |
| | 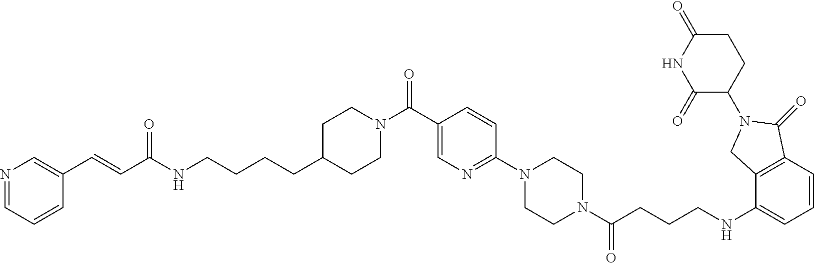 [SMILES: O=C(/C=C/c1cccnc1)NCCCCC1CCN(C(=O)c2ccc(N3CCN(C(=O)CCCNc4cccc5c4CN(C4CCC(=O)NC4=O)C5=O)CC3)nc2)CC1] | (E)-N-(4-(1-(6-(4-(4- ((2-(2,6-dioxopiperidin- 3-yl)-1-oxoisoindolin-4- yl)amino)butanoyl) piperazin-1- yl)nicotinoyl)piperidin- 4-yl)butyl)-3-(pyridin-3- yl)acrylamide | (E)-N-(4-(1-(6-(4-(4- ((2-(2,6- dioxopiperidin-3-yl)-1- oxoisoindolin-4- yl)amino)butanoyl) piperazin-1- yl)nicotinoyl)piperidin- 4-yl)butyl)-3-(pyridin- 3-yl)acrylamide |
| | 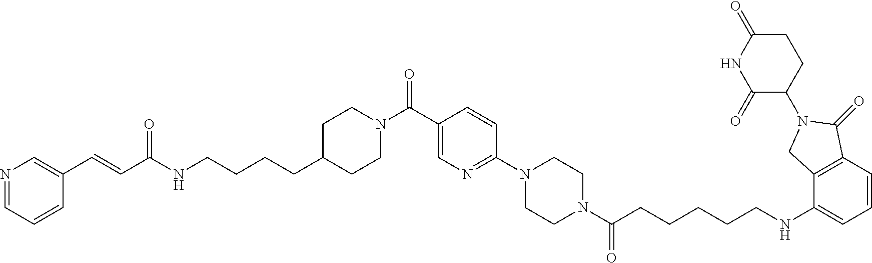 [SMILES: O=C(/C=C/c1cccnc1)NCCCCC1CCN(C(=O)c2ccc(N3CCN(C(=O)CCCCCNc4cccc5c4CN(C4CCC(=O)NC4=O)C5=O)CC3)nc2)CC1] | (E)-N-(4-(1-(6-(4-(6- ((2-(2,6-dioxopiperidin- 3-yl)-1-oxoisoindolin-4- yl)amino)hexanoyl) piperazin-1- yl)nicotinoyl)piperidin- 4-yl)butyl)-3-(pyridin-3- yl)acrylamide | (E)-N-(4-(1-(6-(4-(6- ((2-(2,6- dioxopiperidin-3-yl)-1- oxoisoindolin-4- yl)amino)hexanoyl) piperazin-1- yl)nicotinoyl)piperidin- 4-yl)butyl)-3-(pyridin- 3-yl)acrylamide |
| | 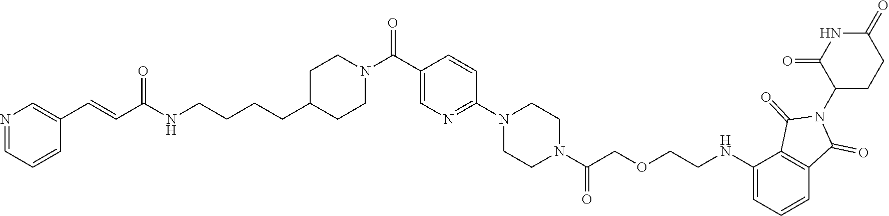 [SMILES: O=C(/C=C/c1cccnc1)NCCCCC1CCN(C(=O)c2ccc(N3CCN(C(=O)COCCNc4cccc5c4C(=O)N(C4CCC(=O)NC4=O)C5=O)CC3)nc2)CC1] | (E)-N-(4-(1-(6-(4-(2-(2- ((2-(2,6-dioxopiperidin- 3-yl)-1-oxoisoindolin-4- yl)amino)ethoxy)acetyl) piperazin-1- yl)nicotinoyl)piperidin- 4-yl)butyl)-3-(pyridin-3- yl)acrylamide | (E)-N-(4-(1-(6-(4-(2- (2-((2-(2,6- dioxopiperidin-3-yl)-1- oxoisoindolin-4- yl)amino)ethoxy)acetyl) piperazin-1- yl)nicotinoyl)piperidin- 4-yl)butyl)-3-(pyridin- 3-yl)acrylamide |
| | 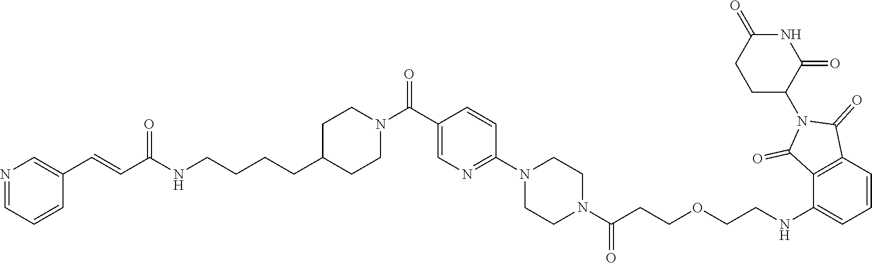 [SMILES: O=C(/C=C/c1cccnc1)NCCCCC1CCN(C(=O)c2ccc(N3CCN(C(=O)CCOCCNc4cccc5c4C(=O)N(C4CCC(=O)NC4=O)C5=O)CC3)nc2)CC1] | (E)-N-(4-(1-(6-(4-(3-(2- ((2-(2,6-dioxopiperidin- 3-yl)-1-oxoisoindolin-4- yl)amino)ethoxy) propanoyl)piperazin-1- yl)nicotinoyl)piperidin- 4-yl)butyl)-3-(pyridin-3- yl)acrylamide | (E)-N-(4-(1-(6-(4-(3- (2-((2-(2,6- dioxopiperidin-3-yl)- 1-oxoisoindolin-4- yl)amino)ethoxy) propanoyl)piperazin-1- yl)nicotinoyl)piperidin- 4-yl)butyl)-3-(pyridin- 3-yl)acrylamide |
| | 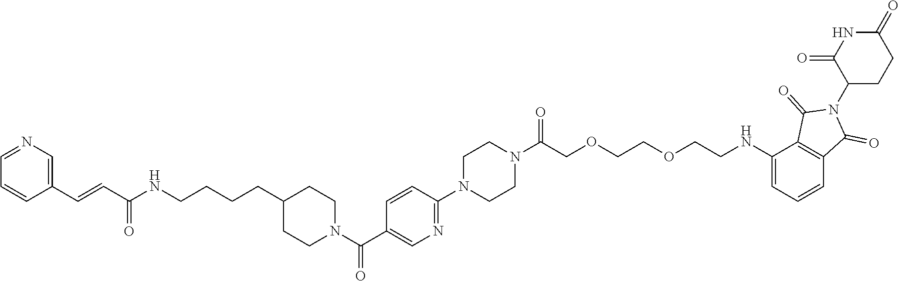 [SMILES: O=C(/C=C/c1cccnc1)NCCCCC1CCN(C(=O)c2ccc(N3CCN(C(=O)COCCOCCNc4cccc5c4C(=O)N(C4CCC(=O)NC4=O)C5=O)CC3)nc2)CC1] | (E)-N-(4-(1-(6-(4-(2-(2- (2-((2-(2,6- dioxopiperidin-3-yl)-1- oxoisoindolin-4- yl)amino)ethoxy)ethoxy) acetyl)piperazin-1- yl)nicotinoyl)piperidin- 4-yl)butyl)-3-(pyridin-3- yl)acrylamide | (E)-N-(4-(1-(6-(4-(2- (2-(2-((2-(2,6- dioxopiperidin-3-yl)-1- oxoisoindolin-4- yl)amino)ethoxy) ethoxy)acetyl) piperazin-1- yl)nicotinoyl)piperidin- 4-yl)butyl)-3-(pyridin- 3-yl)acrylamide |
| | 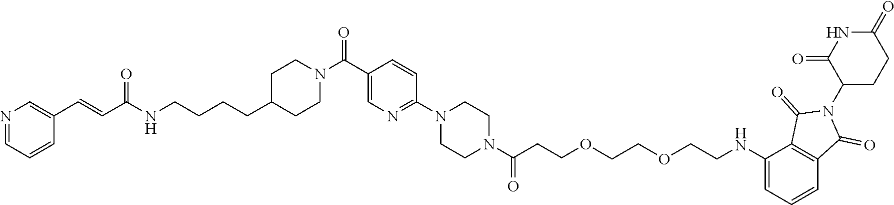 [SMILES: O=C(/C=C/c1cccnc1)NCCCCC1CCN(C(=O)c2ccc(N3CCN(C(=O)CCOCCOCCNc4cccc5c4C(=O)N(C4CCC(=O)NC4=O)C5=O)CC3)nc2)CC1] | (E)-N-(4-(1-(6-(4-(3-(2- (2-((2-(2,6- dioxopiperidin-3-yl)-1- oxoisoindolin-4- yl)amino)ethoxy) ethoxy)propanoyl) piperazin-1- yl)nicotinoyl)piperidin- 4-yl)butyl)-3-(pyridin-3- yl)acrylamide | (E)-N-(4-(1-(6-(4-(3- (2-(2-((2-(2,6- dioxopiperidin-3-yl)- 1-oxoisoindolin-4- yl)amino)ethoxy) ethoxy)propanoyl) piperazin-1- yl)nicotinoyl)piperidin- 4-yl)butyl)-3-(pyridin- 3-yl)acrylamide |
| | 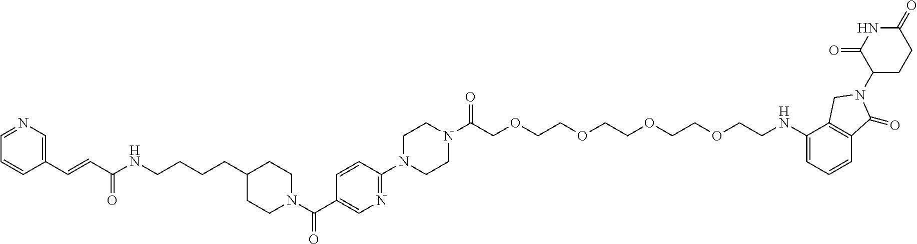 [SMILES: O=C(/C=C/c1cccnc1)NCCCCC1CCN(C(=O)c2ccc(N3CCN(C(=O)COCCOCCOCCOCCNc4cccc5c4CN(C4CCC(=O)NC4=O)C5=O)CC3)nc2)CC1] | (E)-N-(4-(1-(6-(4-(14- ((2-(2,6-dioxopiperidin- 3-yl)-1-oxoisoindolin-4- yl)amino)-3,6,9,12- tetraoxatetradecanoyl) piperazin-1- yl)nicotinoyl)piperidin- 4-yl)butyl)-3-(pyridin-3- yl)acrylamide | (E)-N-(4-(1-(6-(4-(14- ((2-(2,6- dioxopiperidin-3-yl)- 1-oxoisoindolin-4- yl)amino)-3,6,9,12- tetraoxatetradecanoyl) piperazin-1- yl)nicotinoyl)piperidin- 4-yl)butyl)-3-(pyridin- 3-yl)acrylamide |
| | 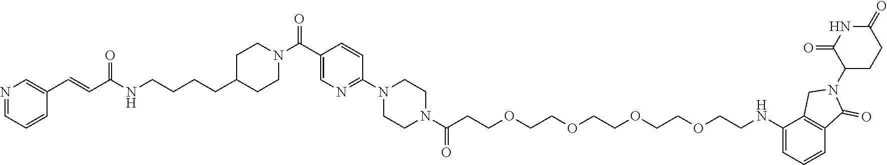 [SMILES: O=C(/C=C/c1cccnc1)NCCCCC1CCN(C(=O)c2ccc(N3CCN(C(=O)CCOCCOCCOCCOCCNc4cccc5c4CN(C4CCC(=O)NC4=O)C5=O)CC3)nc2)CC1] | (E)-N-(4-(1-(6-(4-(1- ((2-(2,6-dioxopiperidin- 3-yl)-1-oxoisoindolin-4- yl)amino)-3,6,9,12- tetraoxapentadecan-15- oyl)piperazin-1- yl)nicotinoyl)piperidin- 4-yl)butyl)-3-(pyridin-3- yl)acrylamide | (E)-N-(4-(1-(6-(4-(1- ((2-(2,6- dioxopiperidin-3-yl)-1- oxoisoindolin-4- yl)amino)-3,6,9,12- tetraoxapentadecan-15- oyl)piperazin-1- yl)nicotinoyl)piperidin- 4-yl)butyl)-3-(pyridin- 3-yl)acrylamide |
| | 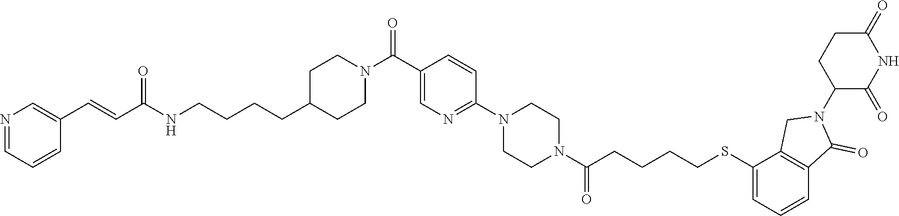 [SMILES: O=C(/C=C/c1cccnc1)NCCCCC1CCN(C(=O)c2ccc(N3CCN(C(=O)CCCCSc4cccc5c4CN(C4CCC(=O)NC4=O)C5=O)CC3)nc2)CC1] | (E)-N-(4-(1-(6-(4-(5- ((2-(2,6-dioxopiperidin- 3-yl)-1-oxoisoindolin-4- yl)thio)pentanoyl) piperazin-1- yl)nicotinoyl)piperidin- 4-yl)butyl)-3-(pyridin-3- yl)acrylamide | (E)-N-(4-(1-(6-(4-(5- ((2-(2,6- dioxopiperidin-3-yl)- 1-oxoisoindolin-4- yl)thio)pentanoyl) piperazin-1- yl)nicotinoyl)piperidin- 4-yl)butyl)-3-(pyridin- 3-yl)acrylamide |
| | 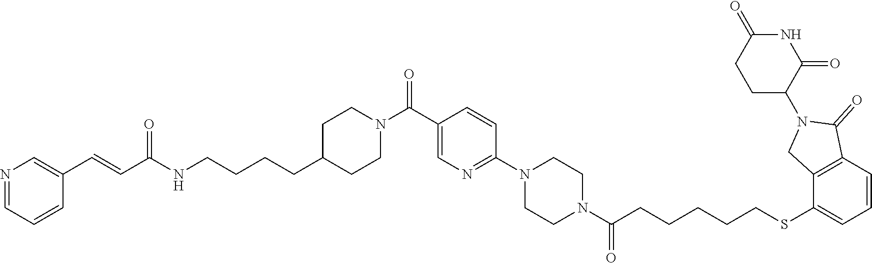 [SMILES: O=C(/C=C/c1cccnc1)NCCCCC1CCN(C(=O)c2ccc(N3CCN(C(=O)CCCCCSc4cccc5c4CN(C4CCC(=O)NC4=O)C5=O)CC3)nc2)CC1] | (E)-N-(4-(1-(6-(4-(6- ((2-(2,6-dioxopiperidin- 3-yl)-1-oxoisoindolin-4- yl)thio)hexanoyl) piperazin-1- yl)nicotinoyl)piperidin- 4-yl)butyl)-3-(pyridin-3- yl)acrylamide | (E)-N-(4-(1-(6-(4-(6- ((2-(2,6- dioxopiperidin-3-yl)- 1-oxoisoindolin-4- yl)thio)hexanoyl) piperazin-1- yl)nicotinoyl)piperidin- 4-yl)butyl)-3-(pyridin- 3-yl)acrylamide |
| | 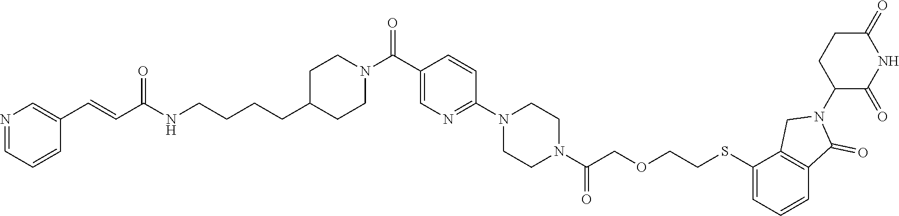 [SMILES: O=C(/C=C/c1cccnc1)NCCCCC1CCN(C(=O)c2ccc(N3CCN(C(=O)COCCSc4cccc5c4CN(C4CCC(=O)NC4=O)C5=O)CC3)nc2)CC1] | (E)-N-(4-(1-(6-(4-(2-(2- ((2-(2,6-dioxopiperidin- 3-yl)-1-oxoisoindolin-4- yl)thio)ethoxy)acetyl) piperazin-1- yl)nicotinoyl)piperidin- 4-yl)butyl)-3-(pyridin-3- yl)acrylamide | (E)-N-(4-(1-(6-(4-(2- (2-((2-(2,6- dioxopiperidin-3-yl)- 1-oxoisoindolin-4- yl)thio)ethoxy)acetyl) piperazin-1- yl)nicotinoyl)piperidin- 4-yl)butyl)-3-(pyridin- 3-yl)acrylamide |
| | 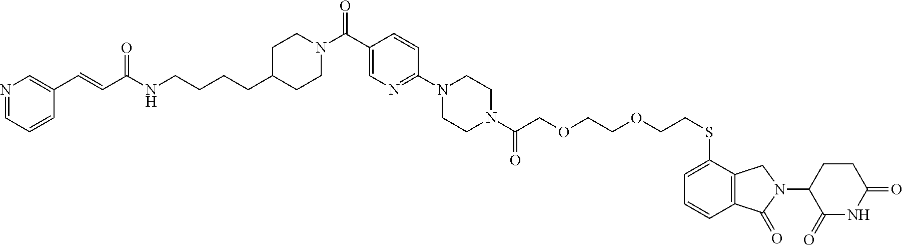 [SMILES: O=C(/C=C/c1cccnc1)NCCCCC1CCN(C(=O)c2ccc(N3CCN(C(=O)COCCOCCSc4cccc5c4CN(C4CCC(=O)NC4=O)C5=O)CC3)nc2)CC1] | (E)-N-(4-(1-(6-(4-(2-(2- (2-((2-(2,6- dioxopiperidin-3-yl)-1- oxoisoindolin-4- yl)thio)ethoxy)ethoxy) acetyl)piperazin-1- yl)nicotinoyl)piperidin- 4-yl)butyl)-3-(pyridin-3- yl)acrylamide | (E)-N-(4-(1-(6-(4-(2- (2-(2-((2-(2,6- dioxopiperidin-3-yl)- 1-oxoisoindolin-4- yl)thio)ethoxy)ethoxy) acetyl)piperazin-1- yl)nicotinoyl)piperidin- 4-yl)butyl)-3-(pyridin- 3-yl)acrylamide |
| | 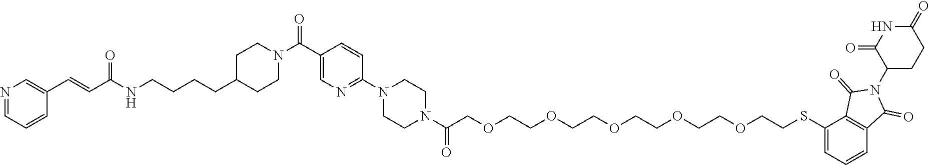 [SMILES: O=C(/C=C/c1cccnc1)NCCCCC1CCN(C(=O)c2ccc(N3CCN(C(=O)COCCOCCOCCOCCOCCSc4cccc5c4C(=O)N(C4CCC(=O)NC4=O)C5=O)CC3)nc2)CC1] | (E)-N-(4-(1-(6-(4-(17- ((2-(2,6-dioxopiperidin- 3-yl)-1-oxoisoindolin-4- yl)thio)-3,6,9,12,15- pentaoxaheptadecanoyl) piperazin-1- yl)nicotinoyl)piperidin- 4-yl)butyl)-3-(pyridin-3- yl)acrylamide | (E)-N-(4-(1-(6-(4-(17- ((2-(2,6- dioxopiperidin-3-yl)- 1-oxoisoindolin-4- yl)thio)-3,6,9,12,15- pentaoxaheptadecanoyl) piperazin-1- yl)nicotinoyl)piperidin- 4-yl)butyl)-3-(pyridin- 3-yl)acrylamide |
| | 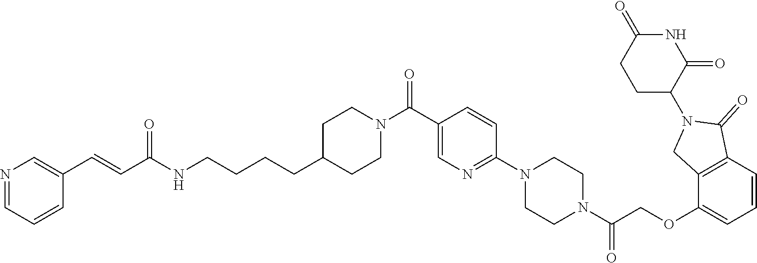 [SMILES: O=C(/C=C/c1cccnc1)NCCCCC1CCN(C(=O)c2ccc(N3CCN(C(=O)COc4cccc5c4CN(C4CCC(=O)NC4=O)C5=O)CC3)nc2)CC1] | (E)-N-(4-(1-(6-(4-(2- ((2-(2,6-dioxopiperidin- 3-yl)-1-oxoisoindolin-4- yl)oxy)acetyl) piperazin-1- yl)nicotinoyl)piperidin- 4-yl)butyl)-3-(pyridin-3- yl)acrylamide | (E)-N-(4-(1-(6-(4-(2- ((2-(2,6- dioxopiperidin-3-yl)-1- oxoisoindolin-4- yl)oxy)acetyl) piperazin-1- yl)nicotinoyl)piperidin- 4-yl)butyl)-3-(pyridin- 3-yl)acrylamide |
| | 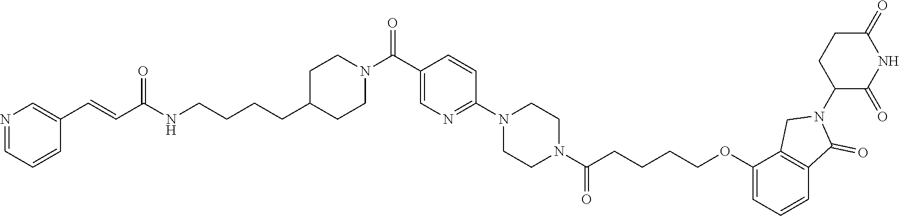 [SMILES: O=C(/C=C/c1cccnc1)NCCCCC1CCN(C(=O)c2ccc(N3CCN(C(=O)CCCCOc4cccc5c4CN(C4CCC(=O)NC4=O)C5=O)CC3)nc2)CC1] | (E)-N-(4-(1-(6-(4-(5- ((2-(2,6-dioxopiperidin- 3-yl)-1-oxoisoindolin-4- yl)oxy)pentanoyl) piperazin-1- yl)nicotinoyl)piperidin- 4-yl)butyl)-3-(pyridin-3- yl)acrylamide | (E)-N-(4-(1-(6-(4-(5- ((2-(2,6- dioxopiperidin-3-yl)-1- oxoisoindolin-4- yl)oxy)pentanoyl) piperazin-1- yl)nicotinoyl)piperidin- 4-yl)butyl)-3-(pyridin- 3-yl)acrylamide |
| | 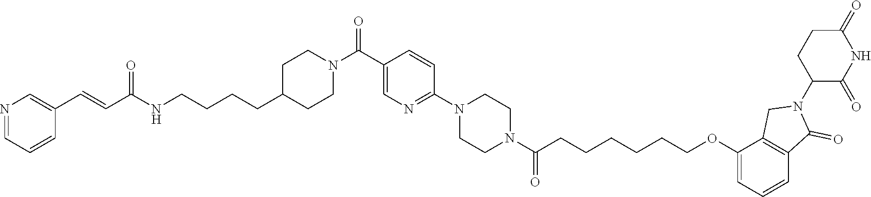 [SMILES: O=C(/C=C/c1cccnc1)NCCCCC1CCN(C(=O)c2ccc(N3CCN(C(=O)CCCCCCOc4cccc5c4CN(C4CCC(=O)NC4=O)C5=O)CC3)nc2)CC1] | (E)-N-(4-(1-(6-(4-(7- ((2-(2,6-dioxopiperidin- 3-yl)-1-oxoisoindolin-4- yl)oxy)heptanoyl) piperazin-1- yl)nicotinoyl)piperidin- 4-yl)butyl)-3-(pyridin-3- yl)acrylamide | (E)-N-(4-(1-(6-(4-(7- ((2-(2,6- dioxopiperidin-3-yl)- 1-oxoisoindolin-4- yl)oxy)heptanoyl) piperazin-1- yl)nicotinoyl)piperidin- 4-yl)butyl)-3-(pyridin- 3-yl)acrylamide |
| | 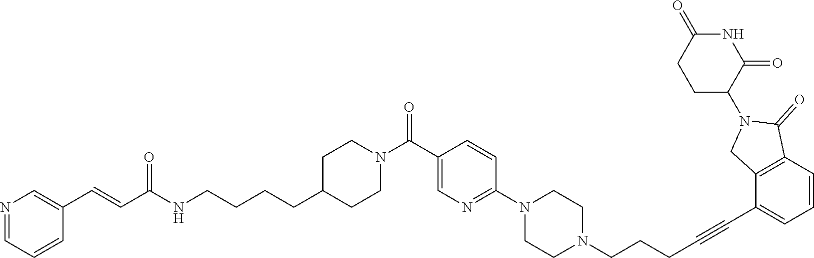 [SMILES: O=C(/C=C/c1cccnc1)NCCCCC1CCN(C(=O)c2ccc(N3CCN(CCCC#Cc4cccc5c4CN(C4CCC(=O)NC4=O)C5=O)CC3)nc2)CC1] | (E)-N-(4-(1-(6-(4-(5-(2- (2,6-dioxopiperidin-3- yl)-1-oxoisoindolin-4- yl)pent-4-yn-1- yl)piperazin-1- yl)nicotinoyl)piperidin- 4-yl)butyl)-3-(pyridin-3- yl)acrylamide | (E)-N-(4-(1-(6-(4-(5- (2-(2,6-dioxopiperidin- 3-yl)-1-oxoisoindolin- 4-yl)pent-4-yn-1- yl)piperazin-1- yl)nicotinoyl)piperidin- 4-yl)butyl)-3-(pyridin- 3-yl)acrylamide |
| | 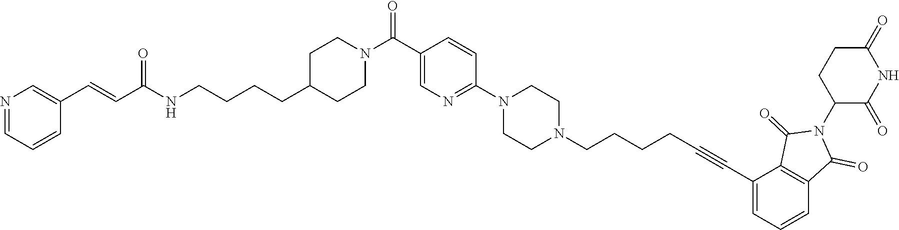 [SMILES: O=C(/C=C/c1cccnc1)NCCCCC1CCN(C(=O)c2ccc(N3CCN(CCCCC#Cc4cccc5c4C(=O)N(C4CCC(=O)NC4=O)C5=O)CC3)nc2)CC1] | (E)-N-(4-(1-(6-(4-(6-(2- (2,6-dioxopiperidin-3- yl)-1-oxoisoindolin-4- yl)hex-5-yn-1- yl)piperazin-1- yl)nicotinoyl)piperidin- 4-yl)butyl)-3-(pyridin-3- yl)acrylamide | (E)-N-(4-(1-(6-(4-(6- (2-(2,6-dioxopiperidin- 3-yl)-1-oxoisoindolin- 4-yl)hex-5-yn-1- yl)piperazin-1- yl)nicotinoyl)piperidin- 4-yl)butyl)-3-(pyridin- 3-yl)acrylamide |
| | 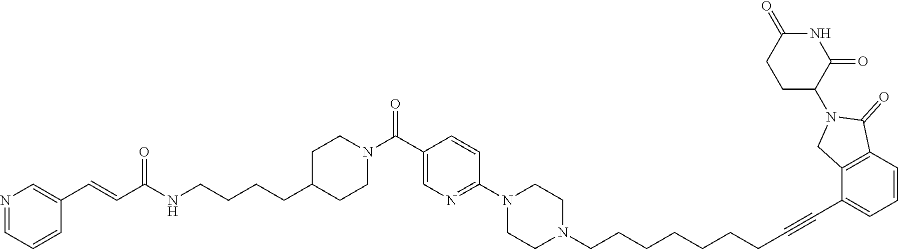 [SMILES: O=C(/C=C/c1cccnc1)NCCCCC1CCN(C(=O)c2ccc(N3CCN(CCCCCCCC#Cc4cccc5c4CN(C4CCC(=O)NC4=O)C5=O)CC3)nc2)CC1] | (E)-N-(4-(1-(6-(4-(9-(2- (2,6-dioxopiperidin-3- yl)-1-oxoisoindolin-4- yl)non-8-yn-1- yl)piperazin-1- yl)nicotinoyl)piperidin- 4-yl)butyl)-3-(pyridin- 3-yl)acrylamide | (E)-N-(4-(1-(6-(4-(9- (2-(2,6-dioxopiperidin- 3-yl)-1-oxoisoindolin- 4-yl)non-8-yn-1- yl)piperazin-1- yl)nicotinoyl)piperidin- 4-yl)butyl)-3-(pyridin- 3-yl)acrylamide |
| | 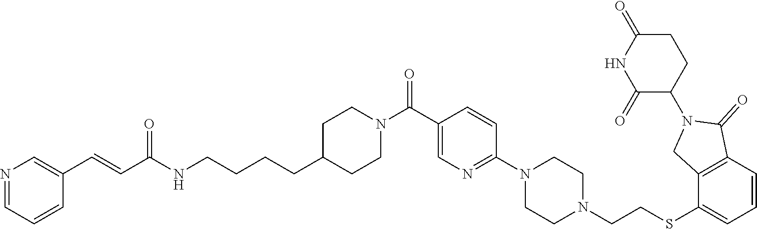 [SMILES: O=C(/C=C/c1cccnc1)NCCCCC1CCN(C(=O)c2ccc(N3CCN(CCSc4cccc5c4CN(C4CCC(=O)NC4=O)C5=O)CC3)nc2)CC1] | (E)-N-(4-(1-(6-(4-(2- ((2-(2,6-dioxopiperidin- 3-yl)-1-oxoisoindolin-4- yl)thio)ethyl) piperazin-1- yl)nicotinoyl)piperidin- 4-yl)butyl)-3-(pyridin-3- yl)acrylamide | (E)-N-(4-(1-(6-(4-(2- ((2-(2,6- dioxopiperidin-3-yl)- 1-oxoisoindolin-4- yl)thio)ethyl) piperazin-1- yl)nicotinoyl)piperidin- 4-yl)butyl)-3-(pyridin- 3-yl)acrylamide |
| | 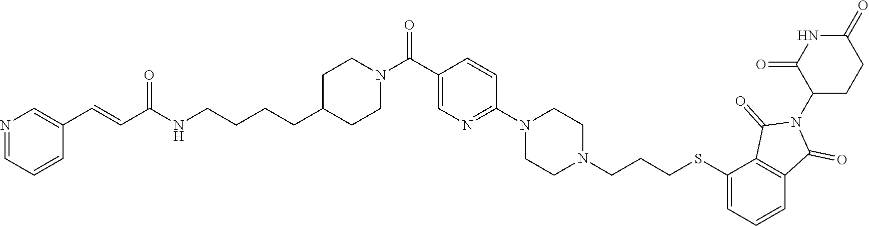 [SMILES: O=C(/C=C/c1cccnc1)NCCCCC1CCN(C(=O)c2ccc(N3CCN(CCCSc4cccc5c4C(=O)N(C4CCC(=O)NC4=O)C5=O)CC3)nc2)CC1] | (E)-N-(4-(1-(6-(4-(3- ((2-(2,6-dioxopiperidin- 3-yl)-1-oxoisoindolin- 4-yl)thio)propyl) piperazin-1- yl)nicotinoyl)piperidin- 4-yl)butyl)-3-(pyridin-3- yl)acrylamide | (E)-N-(4-(1-(6-(4-(3- ((2-(2,6- dioxopiperidin-3-yl)- 1-oxoisoindolin-4- yl)thio)propyl) piperazin-1- yl)nicotinoyl)piperidin- 4-yl)butyl)-3-(pyridin- 3-yl)acrylamide |
| | 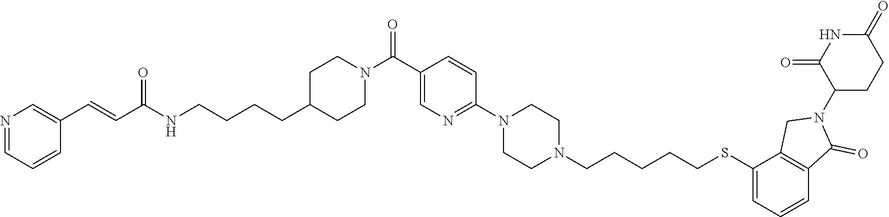 [SMILES: O=C(/C=C/c1cccnc1)NCCCCC1CCN(C(=O)c2ccc(N3CCN(CCCCCSc4cccc5c4CN(C4CCC(=O)NC4=O)C5=O)CC3)nc2)CC1] | (E)-N-(4-(1-(6-(4-(5- ((2-(2,6-dioxopiperidin- 3-yl)-1-oxoisoindolin- 4-yl)thio)pentyl) piperazin-1- yl)nicotinoyl)piperidin- 4-yl)butyl)-3-(pyridin-3- yl)acrylamide | (E)-N-(4-(1-(6-(4-(5- ((2-(2,6- dioxopiperidin-3-yl)- 1-oxoisoindolin-4- yl)thio)pentyl) piperazin-1- yl)nicotinoyl)piperidin- 4-yl)butyl)-3-(pyridin- 3-yl)acrylamide |
| | 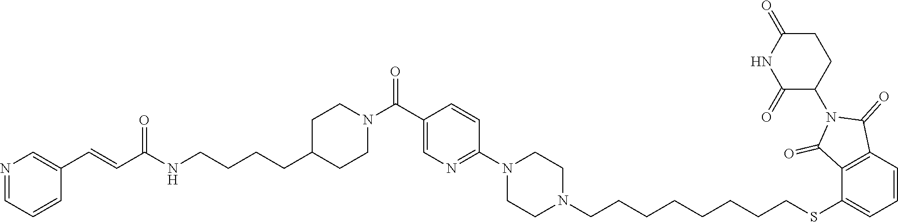 [SMILES: O=C(/C=C/c1cccnc1)NCCCCC1CCN(C(=O)c2ccc(N3CCN(CCCCCCCCSc4cccc5c4C(=O)N(C4CCC(=O)NC4=O)C5=O)CC3)nc2)CC1] | (E)-N-(4-(1-(6-(4-(8- ((2-(2,6-dioxopiperidin- 3-yl)-1-oxoisoindolin- 4-yl)thio)octyl) piperazin-1- yl)nicotinoyl)piperidin- 4-yl)butyl)-3-(pyridin-3- yl)acrylamide | (E)-N-(4-(1-(6-(4-(8- ((2-(2,6- dioxopiperidin-3-yl)- 1-oxoisoindolin-4- yl)thio)octyl) piperazin-1- yl)nicotinoyl)piperidin- 4-yl)butyl)-3-(pyridin- 3-yl)acrylamide |
| | 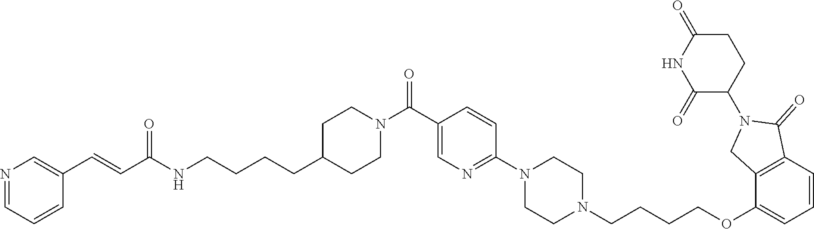 [SMILES: O=C(/C=C/c1cccnc1)NCCCCC1CCN(C(=O)c2ccc(N3CCN(CCCCOc4cccc5c4CN(C4CCC(=O)NC4=O)C5=O)CC3)nc2)CC1] | (E)-N-(4-(1-(6-(4-(4- ((2-(2,6-dioxopiperidin- 3-yl)-1-oxoisoindolin- 4-yl)oxy)butyl) piperazin-1- yl)nicotinoyl)piperidin- 4-yl)butyl)-3-(pyridin-3- yl)acrylamide | (E)-N-(4-(1-(6-(4-(4- ((2-(2,6- dioxopiperidin-3-yl)- 1-oxoisoindolin-4- yl)oxy)butyl) piperazin-1- yl)nicotinoyl)piperidin- 4-yl)butyl)-3-(pyridin- 3-yl)acrylamide |
| | 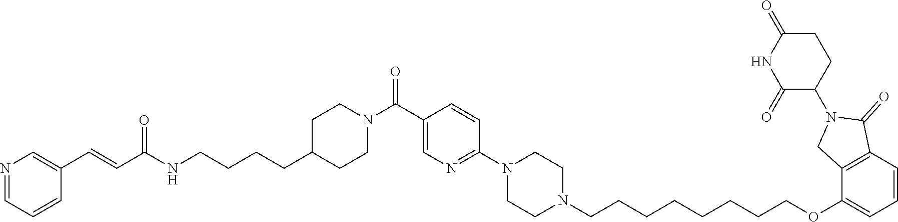 [SMILES: O=C(/C=C/c1cccnc1)NCCCCC1CCN(C(=O)c2ccc(N3CCN(CCCCCCCCOc4cccc5c4CN(C4CCC(=O)NC4=O)C5=O)CC3)nc2)CC1] | (E)-N-(4-(1-(6-(4-(8- ((2-(2,6-dioxopiperidin- 3-yl)-1-oxoisoindolin- 4-yl)oxy)octyl) piperazin-1- yl)nicotinoyl)piperidin- 4-yl)butyl)-3-(pyridin-3- yl)acrylamide | (E)-N-(4-(1-(6-(4-(8- ((2-(2,6- dioxopiperidin-3-yl)- 1-oxoisoindolin-4- yl)oxy)octyl) piperazin-1- yl)nicotinoyl)piperidin- 4-yl)butyl)-3-(pyridin- 3-yl)acrylamide |
| | 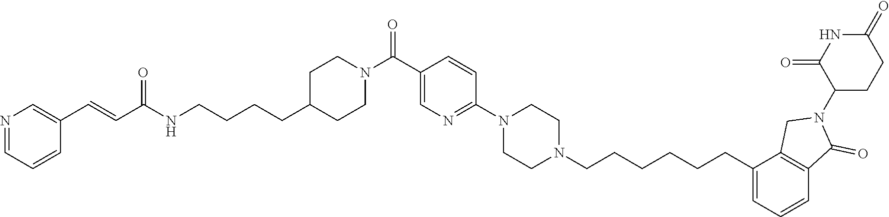 [SMILES: O=C(/C=C/c1cccnc1)NCCCCC1CCN(C(=O)c2ccc(N3CCN(CCCCCCc4cccc5c4CN(C4CCC(=O)NC4=O)C5=O)CC3)nc2)CC1] | (E)-N-(2,6- dioxopiperidin-3-yl)-3- (6-(4-(5-(4-(4-(3- (pyridin-3- yl)acrylamido)butyl) piperidine-1- carbonyl)pyridin-2- yl)piperazin-1- yl)hexyl)benzamide | (E)-N-(2,6- dioxopiperidin-3-yl)- 3-(6-(4-(5-(4-(4-(3- (pyridin-3- yl)acrylamido)butyl) piperidine-1- carbonyl)pyridin-2- yl)piperazin-1- yl)hexyl)benzamide |
| | 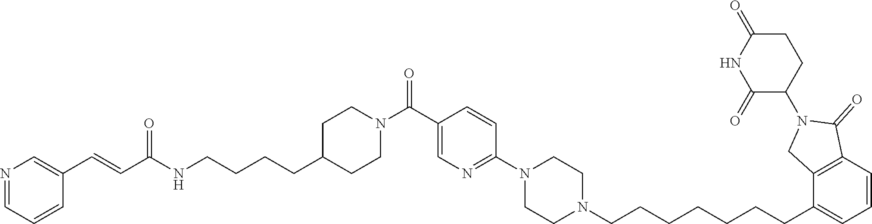 [SMILES: O=C(/C=C/c1cccnc1)NCCCCC1CCN(C(=O)c2ccc(N3CCN(CCCCCCCc4cccc5c4CN(C4CCC(=O)NC4=O)C5=O)CC3)nc2)CC1] | (E)-N-(4-(1-(6-(4-(7-(2- (2,6-dioxopiperidin-3- yl)-1-oxoisoindolin-4- yl)heptyl)piperazin-1- yl)nicotinoyl)piperidin- 4-yl)butyl)-3-(pyridin- 3-yl)acrylamide | (E)-N-(4-(1-(6-(4-(7- (2-(2,6-dioxopiperidin- 3-yl)-1-oxoisoindolin- 4-yl)heptyl) piperazin-1- yl)nicotinoyl)piperidin- 4-yl)butyl)-3-(pyridin- 3-yl)acrylamide |
| | 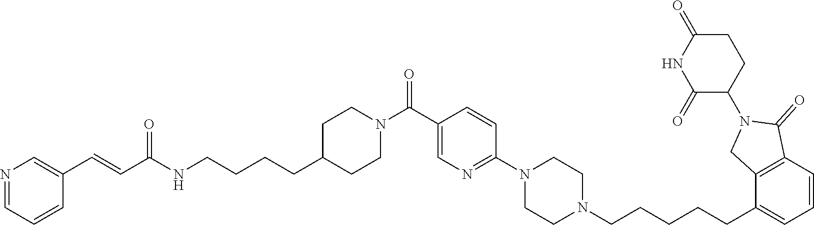 [SMILES: O=C(/C=C/c1cccnc1)NCCCCC1CCN(C(=O)c2ccc(N3CCN(CCCCCc4cccc5c4CN(C4CCC(=O)NC4=O)C5=O)CC3)nc2)CC1] | (E)-N-(4-(1-(6-(4-(5-(2- (2,6-dioxopiperidin-3- yl)-1-oxoisoindolin-4- yl)pentyl)piperazin-1- yl)nicotinoyl)piperidin- 4-yl)butyl)-3-(pyridin- 3-yl)acrylamide | (E)-N-(4-(1-(6-(4-(5- (2-(2,6-dioxopiperidin- 3-yl)-1-oxoisoindolin- 4-yl)pentyl) piperazin-1- yl)nicotinoyl)piperidin- 4-yl)butyl)-3-(pyridin- 3-yl)acrylamide |
| | 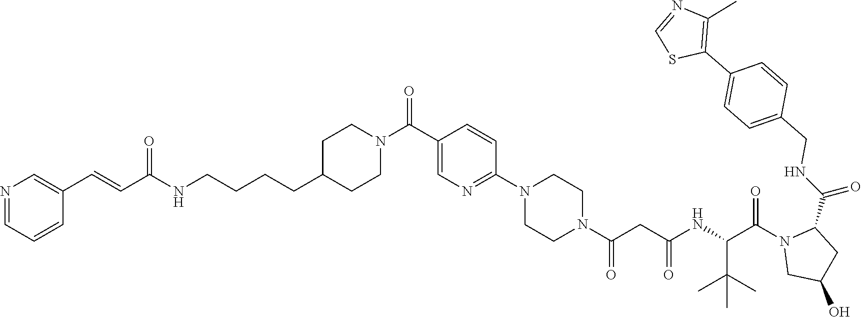 [SMILES: Cc1ncsc1-c1ccc(CNC(=O)[C@@H]2C[C@@H](O)CN2C(=O)[C@@H](NC(=O)CC(=O)N2CCN(c3ccc(C(=O)N4CCC(CCCCNC(=O)/C=C/c5cccnc5)CC4)cn3)CC2)C(C)(C)C)cc1] | (2S,4R)-1-((S)-3,3- dimethyl-2-(3-oxo-3-(4- (5-(4-(4-((E)-3- (pyridin-3- yl)acrylamido)butyl) piperidine-1- carbonyl)pyridin-2- yl)piperazin-1- yl)propanamido) butanoyl)-4-hydroxy -N-(4-(4- methylthiazol-5- yl)benzyl)pyrrolidine-2- carboxamide | (2S,4R)-1-((S)-3,3- dimethyl-2-(3-oxo-3- (4-(5-(4-(4-((E)-3- (pyridin-3- yl)acrylamido)butyl) piperidine-1- carbonyl)pyridin-2- yl)piperazin-1- yl)propanamido) butanoyl)-4- hydroxy-N-(4-(4- methylthiazol-5- yl)benzyl)pyrrolidine- 2-carboxamide |
| | 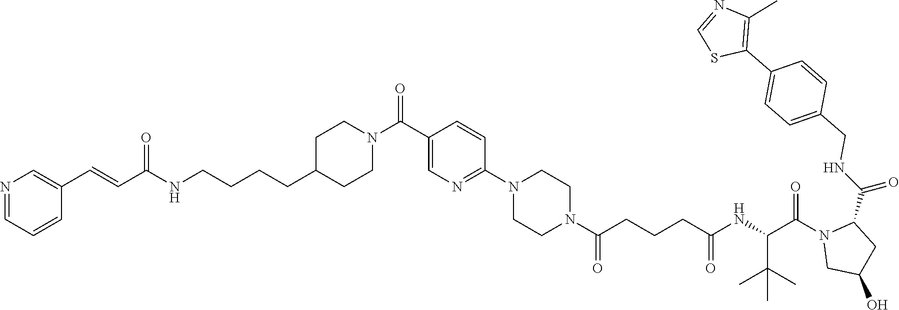 [SMILES: Cc1ncsc1-c1ccc(CNC(=O)[C@@H]2C[C@@H](O)CN2C(=O)[C@@H](NC(=O)CCCC(=O)N2CCN(c3ccc(C(=O)N4CCC(CCCCNC(=O)/C=C/c5cccnc5)CC4)cn3)CC2)C(C)(C)C)cc1] | (2S,4R)-1-((S)-3,3- dimethyl-2-(5-oxo- 5-(4-(5-(4-(4-((E)-3- (pyridin-3- yl)acrylamido)butyl) piperidine-1- carbonyl)pyridin-2- yl)piperazin-1- yl)pentanamido) butanoyl)-4- hydroxy-N-(4-(4- methylthiazol-5- yl)benzyl)pyrrolidine- 2-carboxamide | (2S,4R)-1-((S)-3,3- dimethyl-2-(5-oxo-5- (4-(5-(4-(4-((E)-3- (pyridin-3- yl)acrylamido)butyl) piperidine-1- carbonyl)pyridin-2- yl)piperazin-1- yl)pentanamido) butanoyl)-4- hydroxy-N-(4-(4- methylthiazol-5- yl)benzyl)pyrrolidine- 2-carboxamide |
| | 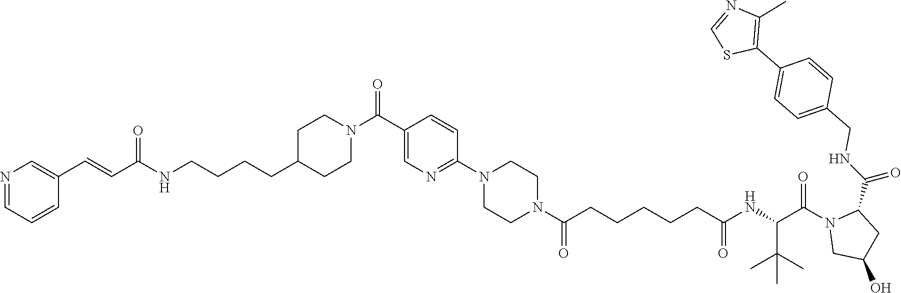 [SMILES: Cc1ncsc1-c1ccc(CNC(=O)[C@@H]2C[C@@H](O)CN2C(=O)[C@@H](NC(=O)CCCCCC(=O)N2CCN(c3ccc(C(=O)N4CCC(CCCCNC(=O)/C=C/c5cccnc5)CC4)cn3)CC2)C(C)(C)C)cc1] | (2S,4R)-1-((S)-3,3- dimethyl-2-(7-oxo- 7-(4-(5-(4-(4-((E)-3- (pyridin-3- yl)acrylamido)butyl) piperidine-1- carbonyl)pyridin-2- yl)piperazin-1- yl)heptanamido) butanoyl)-4- hydroxy-N-(4-(4- methylthiazol-5- yl)benzyl)pyrrolidine- 2-carboxamide | (2S,4R)-1-((S)-3,3- dimethyl-2-(7-oxo-7- (4-(5-(4-(4-((E)-3- (pyridin-3- yl)acrylamido)butyl) piperidine-1- carbonyl)pyridin-2- yl)piperazin-1- yl)heptanamido) butanoyl)-4- hydroxy-N-(4-(4- methylthiazol-5- yl)benzyl)pyrrolidine- 2-carboxamide |
| | 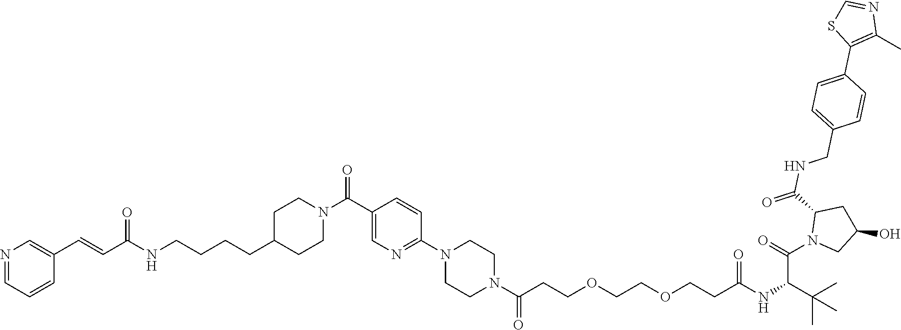 [SMILES: Cc1ncsc1-c1ccc(CNC(=O)[C@@H]2C[C@@H](O)CN2C(=O)[C@@H](NC(=O)CCOCCOCCC(=O)N2CCN(c3ccc(C(=O)N4CCC(CCCCNC(=O)/C=C/c5cccnc5)CC4)cn3)CC2)C(C)(C)C)cc1] | (2S,4R)-1-((S)-3,3- dimethyl-2-(3-(2-(3- oxo-3-(4-(5-(4-(4- ((E)-3-(pyridin-3- yl)acrylamido)butyl) piperidine-1- carbonyl)pyridin-2- yl)piperazin-1- yl)propoxy)ethoxy) propanamido) butanoyl)-4- hydroxy-N-(4-(4- methylthiazol-5- yl)benzyl)pyrrolidine- 2-carboxamide | (2S,4R)-1-((S)-3,3- dimethyl-2-(3-(2-(3- ox0-3-(4-(5-(4-(4- ((E)-3-(pyridin-3- yl)acrylamido)butyl) piperidine-1- carbonyl)pyridin-2- yl)piperazin-1- yl)propoxy)ethoxy) propanamido)butanoyl)- 4-hydroxy-N-(4-(4- methylthiazol-5- yl)benzyl)pyrrolidine- 2-carboxamide |
| | 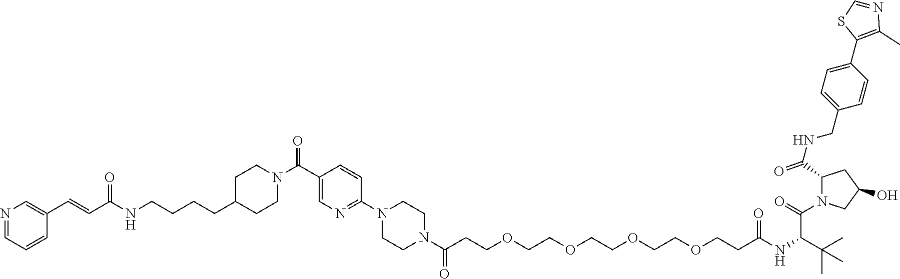 [SMILES: Cc1ncsc1-c1ccc(CNC(=O)[C@@H]2C[C@@H](O)CN2C(=O)[C@@H](NC(=O)CCOCCOCCOCCOCCC(=O)N2CCN(c3ccc(C(=O)N4CCC(CCCCNC(=O)/C=C/c5cccnc5)CC4)cn3)CC2)C(C)(C)C)cc1] | (2S,4R)-1-((S)-2-(tert- butyl)-4, 19-dioxo- 19-(4-(5-(4-(4- ((E)-3-(pyridin-3- yl)acrylamido)butyl) piperidine-1- carbonyl)pyridin-2- yl)piperazin-1-yl)- 7,10,13,16-tetraoxa-3- azanonadecanoyl)-4- hydroxy-N-(4-(4- methylthiazol-5- yl)benzyl)pyrrolidine- 2-carboxamide | (2S,4R)-1-((S)-2-(tert- butyl)-4,19-dioxo-19- (4-(5-(4-(4-((E)-3- (pyridin-3- yl)acrylamido)butyl) piperidine-1- carbonyl)pyridin-2- yl)piperazin-1-yl)- 7,10,13,16-tetraoxa-3- azanonadecanoyl)-4- hydroxy-N-(4-(4- methylthiazol-5- yl)benzyl)pyrrolidine- 2-carboxamide |
| | 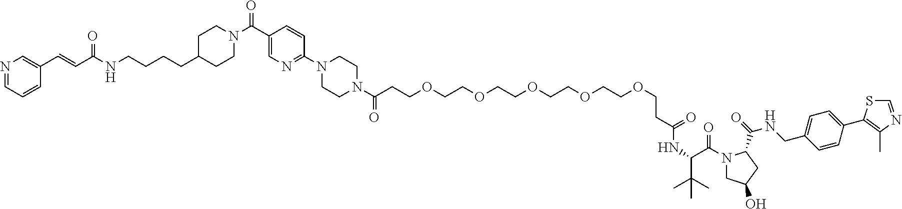 [SMILES: Cc1ncsc1-c1ccc(CNC(=O)[C@@H]2C[C@@H](O)CN2C(=O)[C@@H](NC(=O)CCOCCOCCOCCOCCOCCC(=O)N2CCN(c3ccc(C(=O)N4CCC(CCCCNC(=O)/C=C/c5cccnc5)CC4)cn3)CC2)C(C)(C)C)cc1] | (2S,4R)-1-((S)-2-(tert- butyl)-4,22-dioxo-22- (4-(5-(4-(4-((E)-3- (pyridin-3- yl)acrylamido)butyl) piperidine-1- carbonyl)pyridin-2- yl)piperazin-1-yl)- 7,10,13,16,19- pentaoxa-3- azadocosanoyl)-4- hydroxy-N-(4-(4- methylthiazol-5- | (2S,4R)-1-((S)-2-(tert- butyl)-4,22-dioxo-22- (4-(5-(4-(4-((E)-3- (pyridin-3- yl)acrylamido)butyl) piperidine-1- carbonyl)pyridin-2- yl)piperazin-1-yl)- 7,10,13,16,19- pentaoxa-3- azadocosanoyl)-4- hydroxy-N-(4-(4- methylthiazol-5- |
| | | yl)benzyl)pyrrolidine- | yl)benzyl)pyrrolidine- |
| | | 2-carboxamide | 2-carboxamide |
| | 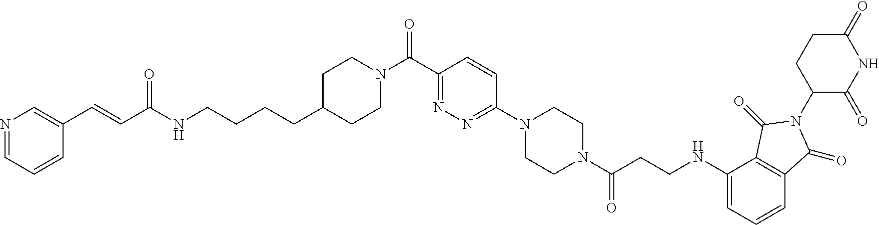 [SMILES: O=C(/C=C/c1cccnc1)NCCCCC1CCN(C(=O)c2ccc(N3CCN(C(=O)CCNc4cccc5c4C(=O)N(C4CCC(=O)NC4=O)C5=O)CC3)nn2)CC1] | (E)-N-(4-(1-(6-(4-(3- ((2-(2,6-dioxopiperidin- 3-yl)-1,3- dioxoisoindolin-4- yl)amino)propanoyl) piperazin-1- yl)pyridazine- 3-carbonyl)piperidin-4- yl)butyl)-3-(pyridin-3- yl)acrylamide | (E)-N-(4-(1-(6-(4-(3- ((2-(2,6- dioxopiperidin-3-yl)- 1,3-dioxoisoindolin-4- yl)amino)propanoyl) piperazin-1- yl)pyridazine-3- carbonyl)piperidin-4- yl)butyl)-3-(pyridin-3- yl)acrylamide |
| | 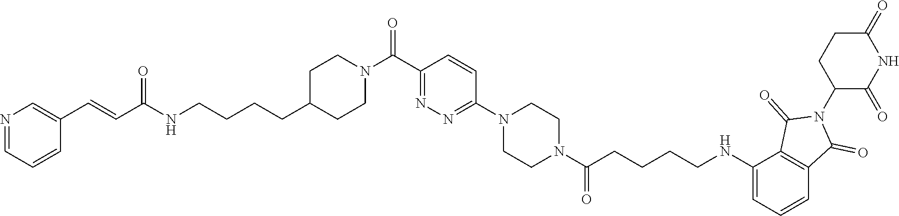 [SMILES: O=C(/C=C/c1cccnc1)NCCCCC1CCN(C(=O)c2ccc(N3CCN(C(=O)CCCCNc4cccc5c4C(=O)N(C4CCC(=O)NC4=O)C5=O)CC3)nn2)CC1] | (E)-N-(4-(1-(6-(4-(5- ((2-(2,6-dioxopiperidin- 3-yl)-1,3- dioxoisoindolin-4- yl)amino)pentanoyl) piperazin-1-yl) pyridazine-3- carbonyl)piperidin-4- yl)butyl)-3-(pyridin-3- yl)acrylamide | (E)-N-(4-(1-(6-(4-(5- ((2-(2,6- dioxopiperidin-3-yl)- 1,3-dioxoisoindolin-4- yl)amino)pentanoyl) piperazin-1-yl) pyridazine- 3-carbonyl)piperidin-4- yl)butyl)-3-(pyridin-3- yl)acrylamide |
| | 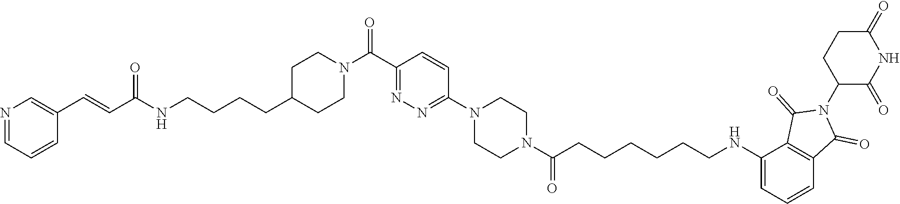 [SMILES: O=C(/C=C/c1cccnc1)NCCCCC1CCN(C(=O)c2ccc(N3CCN(C(=O)CCCCCCNc4cccc5c4C(=O)N(C4CCC(=O)NC4=O)C5=O)CC3)nn2)CC1] | (E)-N-(4-(1-(6-(4-(7- ((2-(2,6-dioxopiperidin- 3-yl)-1,3- dioxoisoindolin-4- yl)amino)heptanoyl) piperazin-1-yl) pyridazine-3- carbonyl)piperidin-4- yl)butyl)-3-(pyridin-3- yl)acrylamide | (E)-N-(4-(1-(6-(4-(7- ((2-(2,6- dioxopiperidin-3-yl)- 1,3-dioxoisoindolin-4- yl)amino)heptanoyl) piperazin-1- yl)pyridazine- 3-carbonyl)piperidin-4- yl)butyl)-3-(pyridin-3- yl)acrylamide |
| | 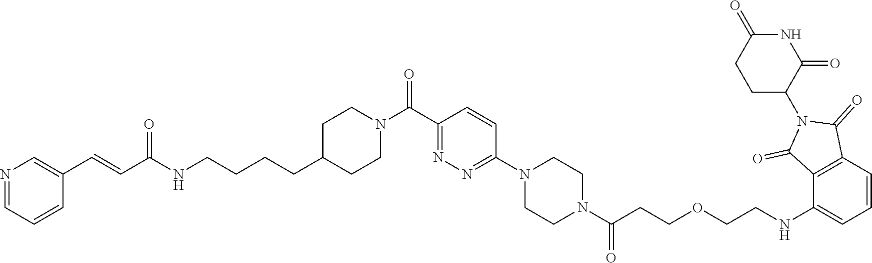 [SMILES: O=C(/C=C/c1cccnc1)NCCCCC1CCN(C(=O)c2ccc(N3CCN(C(=O)CCOCCNc4cccc5c4C(=O)N(C4CCC(=O)NC4=O)C5=O)CC3)nn2)CC1] | (E)-N-(4-(1-(6-(4-(3-(2- ((2-(2,6-dioxopiperidin- 3-yl)-1,3- dioxoisoindolin-4- yl)amino)ethoxy) propanoyl) piperazin-1- yl)pyridazine-3- carbonyl)piperidin-4- yl)butyl)-3-(pyridin-3- yl)acrylamide | (E)-N-(4-(1-(6-(4-(3- (2-((2-(2,6- dioxopiperidin-3-yl)- 1,3-dioxoisoindolin-4- yl)amino)ethoxy) propanoyl)piperazin- 1-yl)pyridazine-3- carbonyl)piperidin-4- yl)butyl)-3-(pyridin-3- yl)acrylamide |
| | 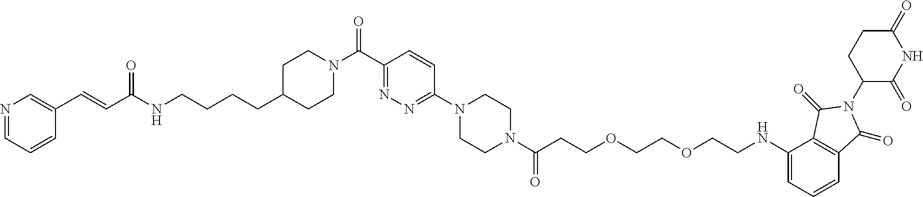 [SMILES: O=C(/C=C/c1cccnc1)NCCCCC1CCN(C(=O)c2ccc(N3CCN(C(=O)CCOCCOCCNc4cccc5c4C(=O)N(C4CCC(=O)NC4=O)C5=O)CC3)nn2)CC1] | (E)-N-(4-(1-(6-(4-(3-(2- (2-((2-(2,6- dioxopiperidin-3-yl)- 1,3-dioxoisoindolin-4- yl)amino)ethoxy) ethoxy)propanoyl) piperazin-1- yl)pyridazine-3- carbonyl)piperidin-4- yl)butyl)-3-(pyridin-3- yl)acrylamide | (E)-N-(4-(1-(6-(4-(3- (2-(2-((2-(2,6- dioxopiperidin-3-yl)- 1,3-dioxoisoindolin-4- yl)amino)ethoxy) ethoxy)propanoyl) piperazin- 1-yl)pyridazine-3- carbonyl)piperidin-4- yl)butyl)-3-(pyridin-3- yl)acrylamide |
| | 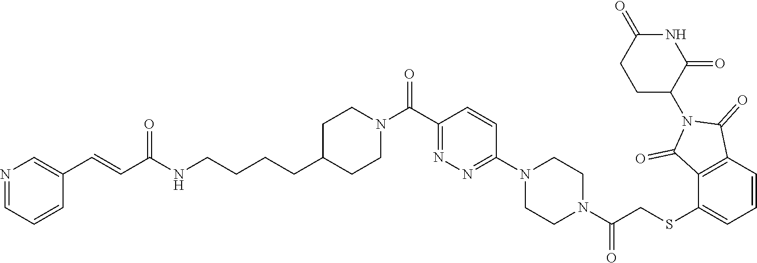 [SMILES: O=C(/C=C/c1cccnc1)NCCCCC1CCN(C(=O)c2ccc(N3CCN(C(=O)CSc4cccc5c4C(=O)N(C4CCC(=O)NC4=O)C5=O)CC3)nn2)CC1] | (E)-N-(4-(1-(6-(4-(2- ((2-(2,6-dioxopiperidin- 3-yl)-1,3- dioxoisoindolin-4- yl)thio)acetyl)piperazin- 1-yl)pyridazine-3- carbonyl)piperidin-4- yl)butyl)-3-(pyridin-3- yl)acrylamide | (E)-N-(4-(1-(6-(4-(2- ((2-(2,6- dioxopiperidin-3-yl)- 1,3-dioxoisoindolin-4- yl)thio)acetyl) piperazin-1- yl)pyridazine-3- carbonyl)piperidin-4- yl)butyl)-3-(pyridin-3- yl)acrylamide |
| | 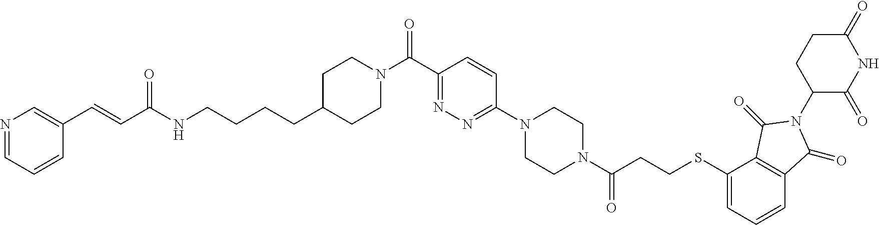 [SMILES: O=C(/C=C/c1cccnc1)NCCCCC1CCN(C(=O)c2ccc(N3CCN(C(=O)CCSc4cccc5c4C(=O)N(C4CCC(=O)NC4=O)C5=O)CC3)nn2)CC1] | (E)-N-(4-(1-(6-(4-(3- ((2-(2,6-dioxopiperidin- 3-yl)-1,3- dioxoisoindolin-4- yl)thio)propanoyl) piperazin-1- yl)pyridazine-3- carbonyl)piperidin-4- yl)butyl)-3-(pyridin-3- yl)acrylamide | (E)-N-(4-(1-(6-(4-(3- ((2-(2,6- dioxopiperidin-3-yl)- 1,3-dioxoisoindolin-4- yl)thio)propanoyl) piperazin-1- yl)pyridazine-3- carbonyl)piperidin-4- yl)butyl)-3-(pyridin-3- yl)acrylamide |
| | 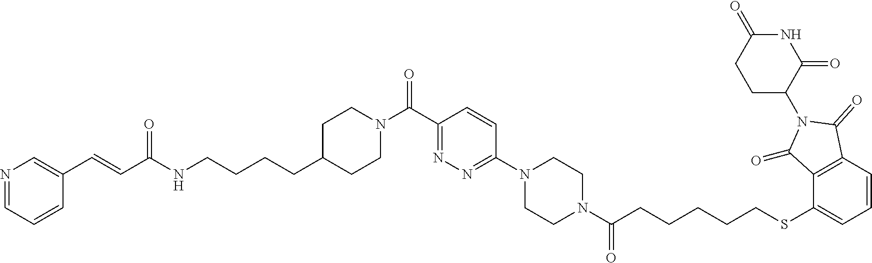 [SMILES: O=C(/C=C/c1cccnc1)NCCCCC1CCN(C(=O)c2ccc(N3CCN(C(=O)CCCCCSc4cccc5c4C(=O)N(C4CCC(=O)NC4=O)C5=O)CC3)nn2)CC1] | (E)-N-(4-(1-(6-(4-(6- ((2-(2,6-dioxopiperidin- 3-yl)-1,3- dioxoisoindolin-4- yl)thio)hexanoyl) piperazin-1- yl)pyridazine-3- carbonyl)piperidin-4- yl)butyl)-3-(pyridin-3- yl)acrylamide | (E)-N-(4-(1-(6-(4-(6- ((2-(2,6- dioxopiperidin-3-yl)- 1,3-dioxoisoindolin-4- yl)thio)hexanoyl) piperazin-1- yl)pyridazine-3- carbonyl)piperidin-4- yl)butyl)-3-(pyridin-3- yl)acrylamide |
| | 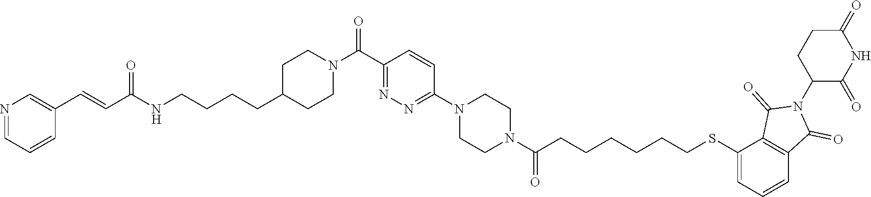 [SMILES: O=C(/C=C/c1cccnc1)NCCCCC1CCN(C(=O)c2ccc(N3CCN(C(=O)CCCCCCSc4cccc5c4C(=O)N(C4CCC(=O)NC4=O)C5=O)CC3)nn2)CC1] | (E)-N-(4-(1-(6-(4-(7- ((2-(2,6-dioxopiperidin- 3-yl)-1,3- dioxoisoindolin-4- yl)thio)heptanoyl) piperazin-1- yl)pyridazine-3- carbonyl)piperidin-4- yl)butyl)-3-(pyridin-3- yl)acrylamide | (E)-N-(4-(1-(6-(4-(7- ((2-(2,6- dioxopiperidin-3-yl)- 1,3-dioxoisoindolin-4- yl)thio)heptanoyl) piperazin-1- yl)pyridazine-3- carbonyl)piperidin-4- yl)butyl)-3-(pyridin-3- yl)acrylamide |
| | 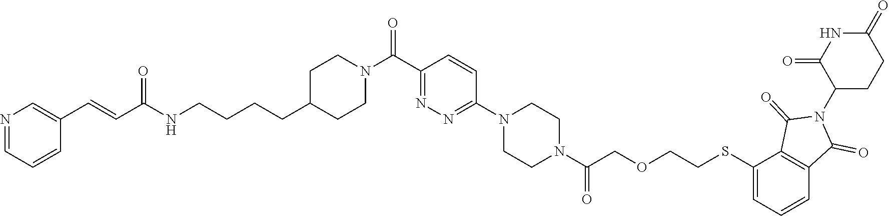 [SMILES: O=C(/C=C/c1cccnc1)NCCCCC1CCN(C(=O)c2ccc(N3CCN(C(=O)COCCSc4cccc5c4C(=O)N(C4CCC(=O)NC4=O)C5=O)CC3)nn2)CC1] | (E)-N-(4-(1-(6-(4-(2-(2- ((2-(2,6-dioxopiperidin- 3-yl)-1,3- dioxoisoindolin-4- yl)thio)ethoxy)acetyl) piperazin-1- yl)pyridazine- 3-carbonyl)piperidin-4- yl)butyl)-3-(pyridin-3- yl)acrylamide | (E)-N-(4-(1-(6-(4-(2- (2-((2-(2,6- dioxopiperidin-3-yl)- 1,3-dioxoisoindolin-4- yl)thio)ethoxy)acetyl) piperazin-1- yl)pyridazine-3- carbonyl)piperidin-4- yl)butyl)-3-(pyridin-3- yl)acrylamide |
| | 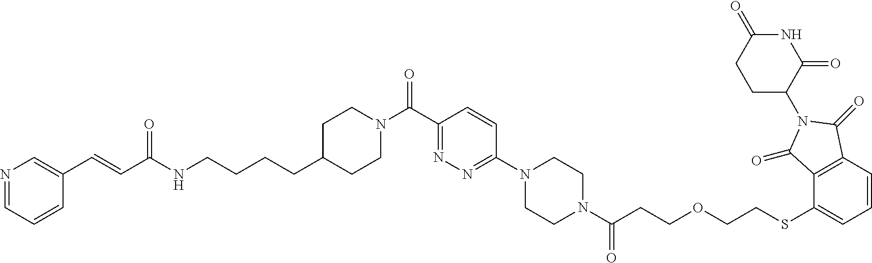 [SMILES: O=C(/C=C/c1cccnc1)NCCCCC1CCN(C(=O)c2ccc(N3CCN(C(=O)CCOCCSc4cccc5c4C(=O)N(C4CCC(=O)NC4=O)C5=O)CC3)nn2)CC1] | (E)-N-(4-(1-(6-(4-(3-(2- ((2-(2,6-dioxopiperidin- 3-yl)-1,3- dioxoisoindolin-4- yl)thio)ethoxy) propanoyl)piperazin-1- yl)pyridazine-3- carbonyl)piperidin-4- yl)butyl)-3-(pyridin-3- yl)acrylamide | (E)-N-(4-(1-(6-(4-(3- (2-((2-(2,6- dioxopiperidin-3-yl)- 1,3-dioxoisoindolin- 4-yl)thio)ethoxy) propanoyl)piperazin- 1-yl)pyridazine-3- carbonyl)piperidin-4- yl)butyl)-3-(pyridin-3- yl)acrylamide |
| | 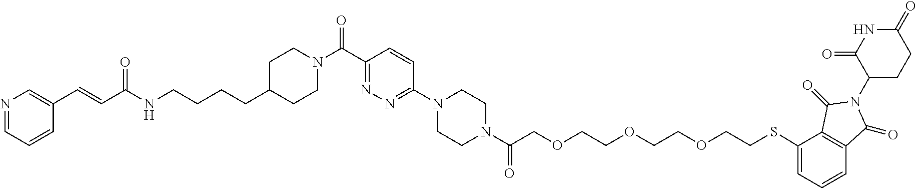 [SMILES: O=C(/C=C/c1cccnc1)NCCCCC1CCN(C(=O)c2ccc(N3CCN(C(=O)COCCOCCOCCSc4cccc5c4C(=O)N(C4CCC(=O)NC4=O)C5=O)CC3)nn2)CC1] | (E)-N-(4-(1-(6-(4-(2-(2- (2-(2-((2-(2,6- dioxopiperidin-3-yl)- 1,3-dioxoisoindolin-4- yl)thio)ethoxy)ethoxy) ethoxy)acetyl) piperazin-1- yl)pyridazine-3- carbonyl)piperidin-4- yl)butyl)-3-(pyridin-3- yl)acrylamide | (E)-N-(4-(1-(6-(4-(2- (2-(2-(2-((2-(2,6- dioxopiperidin-3-yl)- 1,3-dioxoisoindolin-4- yl)thio)ethoxy)ethoxy) ethoxy)acetyl) piperazin- 1-yl)pyridazine-3- carbonyl)piperidin-4- yl)butyl)-3-(pyridin-3- yl)acrylamide |
| | 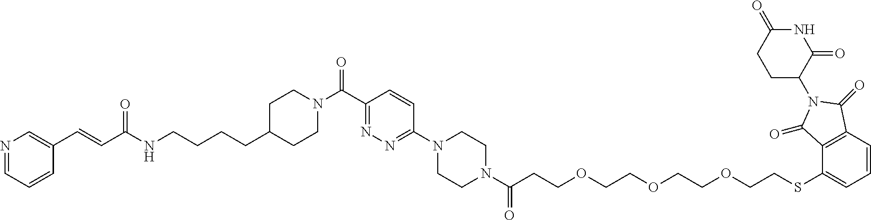 [SMILES: O=C(/C=C/c1cccnc1)NCCCCC1CCN(C(=O)c2ccc(N3CCN(C(=O)CCOCCOCCOCCSc4cccc5c4C(=O)N(C4CCC(=O)NC4=O)C5=O)CC3)nn2)CC1] | (E)-N-(4-(1-(6-(4-(3-(2- (2-(2-((2-(2,6- dioxopiperidin-3-yl)- 1,3-dioxoisoindolin-4- yl)thio)ethoxy)ethoxy) ethoxy)propanoyl) piperazin-1- yl)pyridazine-3- carbonyl)piperidin-4- yl)butyl)-3-(pyridin-3- yl)acrylamide | (E)-N-(4-(1-(6-(4-(3- (2-(2-(2-((2-(2,6- dioxopiperidin-3-yl)- 1,3-dioxoisoindolin-4- yl)thio)ethoxy)ethoxy) ethoxy)propanoyl) piperazin-1- yl)pyridazine-3- carbonyl)piperidin-4- yl)butyl)-3-(pyridin-3- yl)acrylamide |
| | 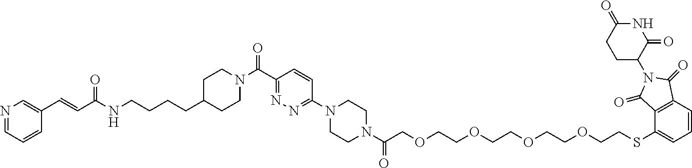 [SMILES: O=C(/C=C/c1cccnc1)NCCCCC1CCN(C(=O)c2ccc(N3CCN(C(=O)COCCOCCOCCOCCSc4cccc5c4C(=O)N(C4CCC(=O)NC4=O)C5=O)CC3)nn2)CC1] | (E)-N-(4-(1-(6-(4-(14- ((2-(2,6-dioxopiperidin- 3-yl)-1,3- dioxoisoindolin-4- yl)thio)-3,6,9,12- tetraoxatetradecanoyl) piperazin-1- yl)pyridazine- 3-carbonyl)piperidin-4- yl)butyl)-3-(pyridin-3- yl)acrylamide | (E)-N-(4-(1-(6-(4-(14- ((2-(2,6- dioxopiperidin-3-yl)- 1,3-dioxoisoindolin-4- yl)thio)-3,6,9,12- tetraoxatetradecanoyl) piperazin-1- yl)pyridazine-3- carbonyl)piperidin-4- yl)butyl)-3-(pyridin-3- yl)acrylamide |
| | 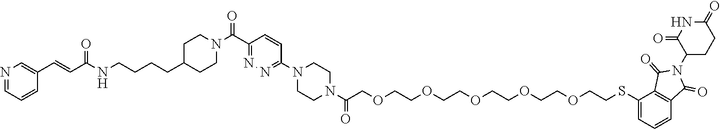 [SMILES: O=C(/C=C/c1cccnc1)NCCCCC1CCN(C(=O)c2ccc(N3CCN(C(=O)COCCOCCOCCOCCOCCSc4cccc5c4C(=O)N(C4CCC(=O)NC4=O)C5=O)CC3)nn2)CC1] | (E)-N-(4-(1-(6-(4-(17- ((2-(2,6-dioxopiperidin- 3-yl)-1,3- dioxoisoindolin-4- yl)thio)-3,6,9,12,15- pentaoxaheptadecanoyl) piperazin-1- yl)pyridazine-3- | (E)-N-(4-(1-(6-(4-(17- ((2-(2,6- dioxopiperidin-3-yl)- 1,3-dioxoisoindolin-4- yl)thio)-3,6,9,12,15- pentaoxaheptadecanoyl) piperazin-1- yl)pyridazine-3- |
| | | carbonyl)piperidin-4- | carbonyl)piperidin-4- |
| | | yl)butyl)-3-(pyridin-3- | yl)butyl)-3-(pyridin-3- |
| | | yl)acrylamide | yl)acrylamide |
| | 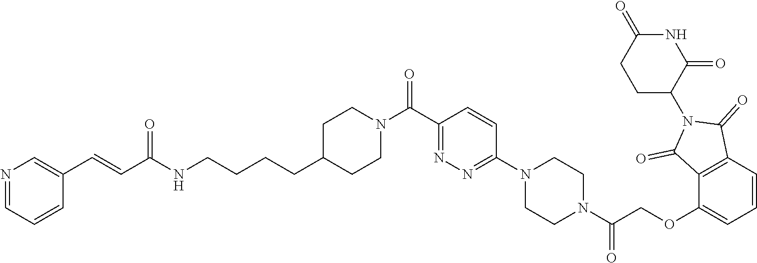 [SMILES: O=C(/C=C/c1cccnc1)NCCCCC1CCN(C(=O)c2ccc(N3CCN(C(=O)COc4cccc5c4C(=O)N(C4CCC(=O)NC4=O)C5=O)CC3)nn2)CC1] | (E)-N-(4-(1-(6-(4-(2- ((2-(2,6-dioxopiperidin- 3-yl)-1,3- dioxoisoindolin-4- yl)oxy)acetyl)piperazin- 1-yl)pyridazine-3- carbonyl)piperidin-4- yl)butyl)-3-(pyridin-3- yl)acrylamide | (E)-N-(4-(1-(6-(4-(2- ((2-(2,6- dioxopiperidin-3-yl)- 1,3-dioxoisoindolin-4- yl)oxy)acetyl) piperazin-1- yl)pyridazine-3- carbonyl)piperidin-4- yl)butyl)-3-(pyridin-3- yl)acrylamide |
| | 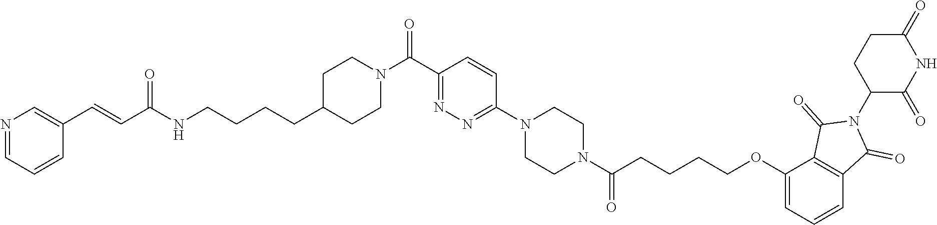 [SMILES: O=C(/C=C/c1cccnc1)NCCCCC1CCN(C(=O)c2ccc(N3CCN(C(=O)CCCCOc4cccc5c4C(=O)N(C4CCC(=O)NC4=O)C5=O)CC3)nn2)CC1] | (E)-N-(4-(1-(6-(4-(5- ((2-(2,6-dioxopiperidin- 3-yl)-1,3- dioxoisoindolin-4- yl)oxy)pentanoyl) piperazin-1- yl)pyridazine-3- carbonyl)piperidin-4- yl)butyl)-3-(pyridin-3- yl)acrylamide | (E)-N-(4-(1-(6-(4-(5- ((2-(2,6- dioxopiperidin-3-yl)- 1,3-dioxoisoindolin-4- yl)oxy)pentanoyl) piperazin-1- yl)pyridazine-3- carbonyl)piperidin-4- yl)butyl)-3-(pyridin-3- yl)acrylamide |
| | 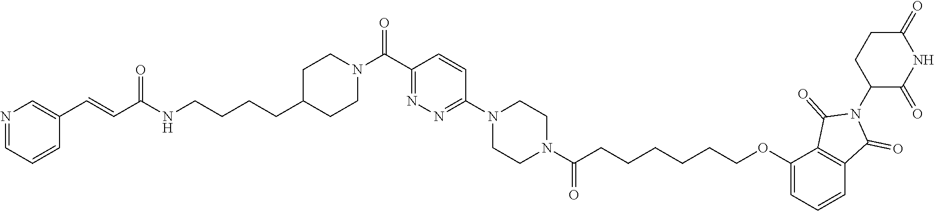 [SMILES: O=C(/C=C/c1cccnc1)NCCCCC1CCN(C(=O)c2ccc(N3CCN(C(=O)CCCCCCOc4cccc5c4C(=O)N(C4CCC(=O)NC4=O)C5=O)CC3)nn2)CC1] | (E)-N-(4-(1-(6-(4-(7- ((2-(2,6-dioxopiperidin- 3-yl)-1,3- dioxoisoindolin-4- yl)oxy)heptanoyl) piperazin-1- yl)pyridazine-3- carbonyl)piperidin-4- yl)butyl)-3-(pyridin-3- yl)acrylamide | (E)-N-(4-(1-(6-(4-(7- ((2-(2,6- dioxopiperidin-3-yl)- 1,3-dioxoisoindolin-4- yl)oxy)heptanoyl) piperazin-1- yl)pyridazine-3- carbonyl)piperidin-4- yl)butyl)-3-(pyridin-3- yl)acrylamide |
| | 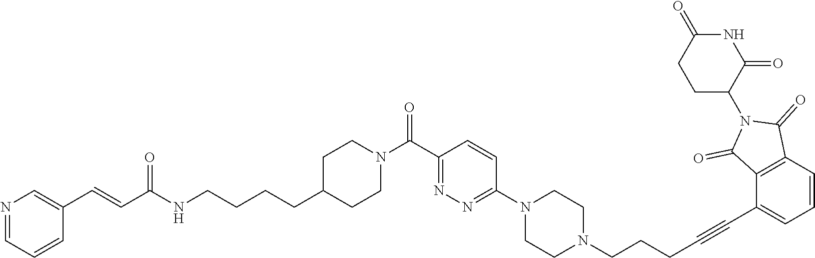 [SMILES: O=C(/C=C/c1cccnc1)NCCCCC1CCN(C(=O)c2ccc(N3CCN(CCCC#Cc4cccc5c4C(=O)N(C4CCC(=O)NC4=O)C5=O)CC3)nn2)CC1] | (E)-N-(4-(1-(6-(4-(5-(2- (2,6-dioxopiperidin-3- yl)-1,3-dioxoisoindolin- 4-yl)pent-4-yn-1- yl)piperazin-1- yl)pyridazine-3- carbonyl)piperidin-4- yl)butyl)-3-(pyridin-3- yl)acrylamide | (E)-N-(4-(1-(6-(4-(5- (2-(2,6-dioxopiperidin- 3-yl)-1,3- dioxoisoindolin-4- yl)pent-4-yn-1- yl)piperazin-1- yl)pyridazine-3- carbonyl)piperidin-4- yl)butyl)-3-(pyridin-3- yl)acrylamide |
| | 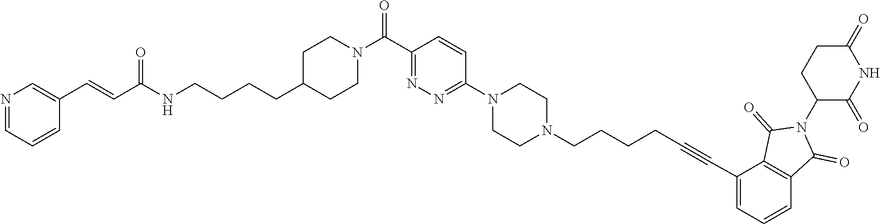 [SMILES: O=C(/C=C/c1cccnc1)NCCCCC1CCN(C(=O)c2ccc(N3CCN(CCCCC#Cc4cccc5c4C(=O)N(C4CCC(=O)NC4=O)C5=O)CC3)nn2)CC1] | (E)-N-(4-(1-(6-(4-(6-(2- (2,6-dioxopiperidin-3- yl)-1,3-dioxoisoindolin- 4-yl)hex-5-yn-1- yl)piperazin-1- yl)pyridazine-3- carbonyl)piperidin-4- yl)butyl)-3-(pyridin-3- yl)acrylamide | (E)-N-(4-(1-(6-(4-(6- (2-(2,6-dioxopiperidin- 3-yl)-1,3- dioxoisoindolin-4- yl)hex-5-yn-1- yl)piperazin-1- yl)pyridazine-3- carbonyl)piperidin-4- yl)butyl)-3-(pyridin-3- yl)acrylamide |
| | 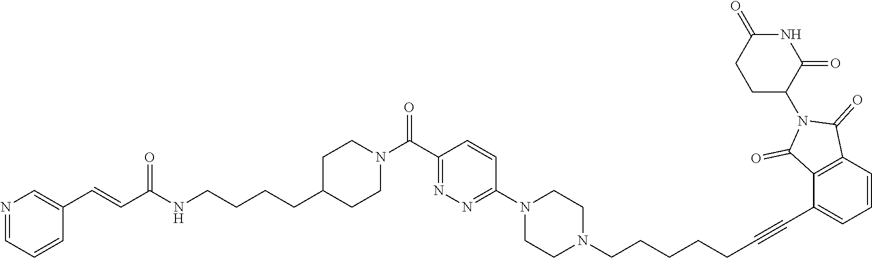 [SMILES: O=C(/C=C/c1cccnc1)NCCCCC1CCN(C(=O)c2ccc(N3CCN(CCCCCC#Cc4cccc5c4C(=O)N(C4CCC(=O)NC4=O)C5=O)CC3)nn2)CC1] | (E)-N-(4-(1-(6-(4-(7-(2- (2,6-dioxopiperidin-3- yl)-1,3-dioxoisoindolin- 4-yl)hept-6-yn-1- yl)piperazin-1- yl)pyridazine-3- carbonyl)piperidin-4- yl)butyl)-3-(pyridin-3- yl)acrylamide | (E)-N-(4-(1-(6-(4-(7- (2-(2,6-dioxopiperidin- 3-yl)-1,3- dioxoisoindolin-4- yl)hept-6-yn-1- yl)piperazin-1- yl)pyridazine-3- carbonyl)piperidin-4- yl)butyl)-3-(pyridin-3- yl)acrylamide |
| | 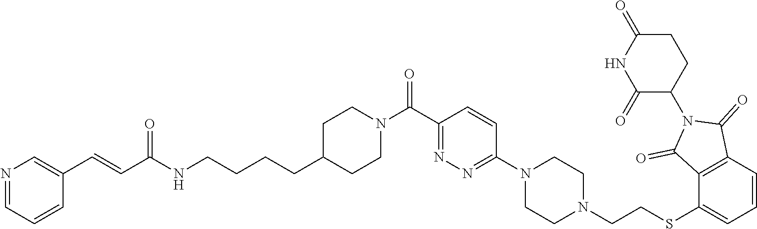 [SMILES: O=C(/C=C/c1cccnc1)NCCCCC1CCN(C(=O)c2ccc(N3CCN(CCSc4cccc5c4C(=O)N(C4CCC(=O)NC4=O)C5=O)CC3)nn2)CC1] | (E)-N-(4-(1-(6-(4-(2- ((2-(2,6-dioxopiperidin- 3-yl)-1,3- dioxoisoindolin-4- yl)thio)ethyl)piperazin- 1-yl)pyridazine-3- carbonyl)piperidin-4- yl)butyl)-3-(pyridin-3- yl)acrylamide | (E)-N-(4-(1-(6-(4-(2- ((2-(2,6- dioxopiperidin-3-yl)- 1,3-dioxoisoindolin-4- yl)thio)ethyl)piperazin- 1-yl)pyridazine-3- carbonyl)piperidin-4- yl)butyl)-3-(pyridin-3- yl)acrylamide |
| | 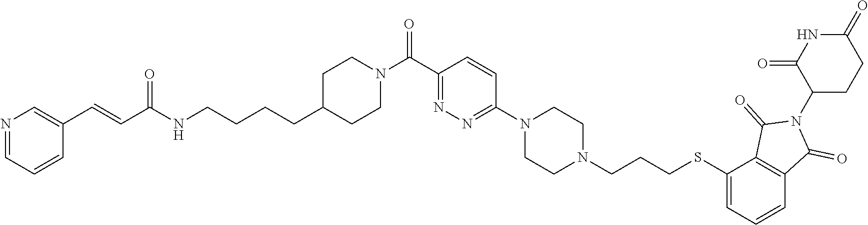 [SMILES: O=C(/C=C/c1cccnc1)NCCCCC1CCN(C(=O)c2ccc(N3CCN(CCCSc4cccc5c4C(=O)N(C4CCC(=O)NC4=O)C5=O)CC3)nn2)CC1] | (E)-N-(4-(1-(6-(4-(3- ((2-(2,6-dioxopiperidin- 3-yl)-1,3- dioxoisoindolin-4- yl)thio)propyl) piperazin-1- yl)pyridazine-3- carbonyl)piperidin-4- yl)butyl)-3-(pyridin-3- yl)acrylamide | (E)-N-(4-(1-(6-(4-(3- ((2-(2,6- dioxopiperidin-3-yl)- 1,3-dioxoisoindolin-4- yl)thio)propyl) piperazin-1- yl)pyridazine-3- carbonyl)piperidin-4- yl)butyl)-3-(pyridin-3- yl)acrylamide |
| | 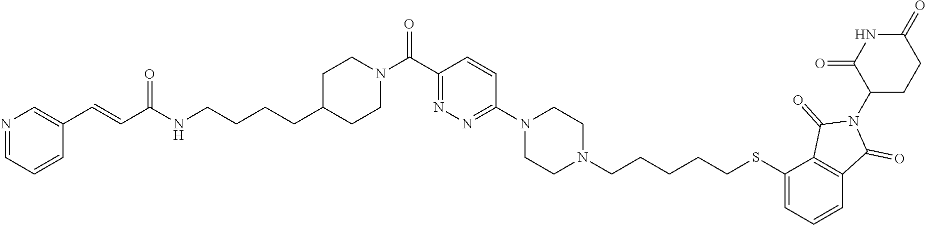 [SMILES: O=C(/C=C/c1cccnc1)NCCCCC1CCN(C(=O)c2ccc(N3CCN(CCCCCSc4cccc5c4C(=O)N(C4CCC(=O)NC4=O)C5=O)CC3)nn2)CC1] | (E)-N-(4-(1-(6-(4-(5- ((2-(2,6-dioxopiperidin- 3-yl)-1,3- dioxoisoindolin-4- yl)thio)pentyl) piperazin-1- yl)pyridazine-3- carbonyl)piperidin-4- yl)butyl)-3-(pyridin-3- yl)acrylamide | (E)-N-(4-(1-(6-(4-(5- ((2-(2,6- dioxopiperidin-3-yl)- 1,3-dioxoisoindolin-4- yl)thio)pentyl) piperazin-1- yl)pyridazine-3- carbonyl)piperidin-4- yl)butyl)-3-(pyridin-3- yl)acrylamide |
| | 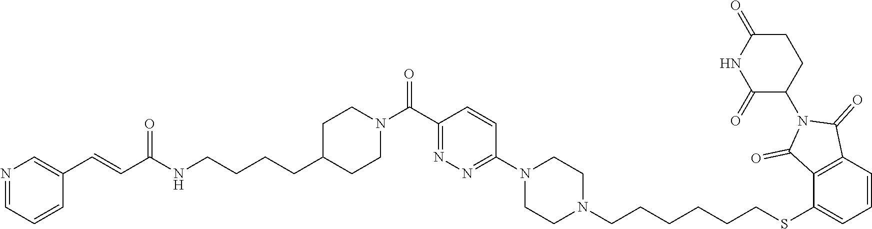 [SMILES: O=C(/C=C/c1cccnc1)NCCCCC1CCN(C(=O)c2ccc(N3CCN(CCCCCCSc4cccc5c4C(=O)N(C4CCC(=O)NC4=O)C5=O)CC3)nn2)CC1] | (E)-N-(4-(1-(6-(4-(6- ((2-(2,6-dioxopiperidin- 3-yl)-1,3- dioxoisoindolin-4- yl)thio)hexyl)piperazin- 1-yl)pyridazine-3- carbonyl)piperidin-4- yl)butyl)-3-(pyridin-3- yl)acrylamide | (E)-N-(4-(1-(6-(4-(6- ((2-(2,6- dioxopiperidin-3-yl)- 1,3-dioxoisoindolin-4- yl)thio)hexyl) piperazin-1- yl)pyridazine-3- carbonyl)piperidin-4- yl)butyl)-3-(pyridin-3- yl)acrylamide |
| | 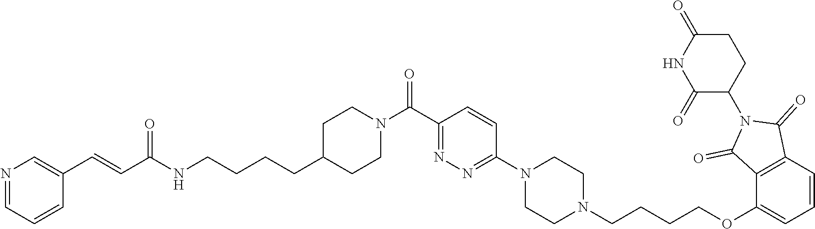 [SMILES: O=C(/C=C/c1cccnc1)NCCCCC1CCN(C(=O)c2ccc(N3CCN(CCCCOc4cccc5c4C(=O)N(C4CCC(=O)NC4=O)C5=O)CC3)nn2)CC1] | (E)-N-(4-(1-(6-(4-(4- ((2-(2,6-dioxopiperidin- 3-yl)-1,3- dioxoisoindolin-4- yl)oxy)butyl)piperazin- 1-yl)pyridazine-3- carbonyl)piperidin-4- yl)butyl)-3-(pyridin-3- yl)acrylamide | (E)-N-(4-(1-(6-(4-(4- ((2-(2,6- dioxopiperidin-3-yl)- 1,3-dioxoisoindolin-4- yl)oxy)butyl)piperazin- 1-yl)pyridazine-3- carbonyl)piperidin-4- yl)butyl)-3-(pyridin-3- yl)acrylamide |
| | 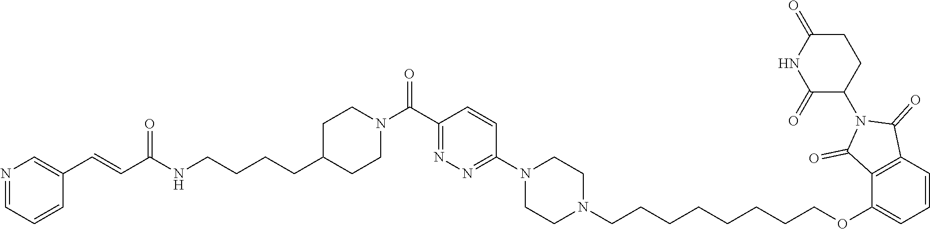 [SMILES: O=C(/C=C/c1cccnc1)NCCCCC1CCN(C(=O)c2ccc(N3CCN(CCCCCCCCOc4cccc5c4C(=O)N(C4CCC(=O)NC4=O)C5=O)CC3)nn2)CC1] | (E)-N-(4-(1-(6-(4-(8- ((2-(2,6-dioxopiperidin- 3-yl)-1,3- dioxoisoindolin-4- yl)oxy)octyl)piperazin- 1-yl)pyridazine-3- carbonyl)piperidin-4- yl)butyl)-3-(pyridin-3- yl)acrylamide | (E)-N-(4-(1-(6-(4-(8- ((2-(2,6- dioxopiperidin-3-yl)- 1,3-dioxoisoindolin-4- yl)oxy)octyl)piperazin- 1-yl)pyridazine-3- carbonyl)piperidin-4- yl)butyl)-3-(pyridin-3- yl)acrylamide |
| | 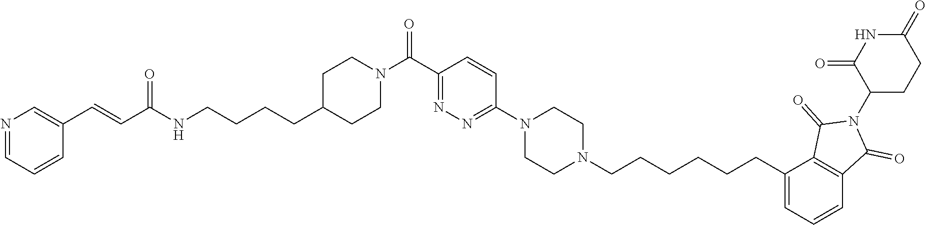 [SMILES: O=C(/C=C/c1cccnc1)NCCCCC1CCN(C(=O)c2ccc(N3CCN(CCCCCCc4cccc5c4C(=O)N(C4CCC(=O)NC4=O)C5=O)CC3)nn2)CC1] | (E)-N-(4-(1-(6-(4-(6-(2- (2,6-dioxopiperidin-3- yl)-1,3-dioxoisoindolin- 4-yl)hexyl)piperazin-1- yl)pyridazine-3- carbonyl)piperidin-4- yl)butyl)-3-(pyridin-3- yl)acrylamide | (E)-N-(4-(1-(6-(4-(6- (2-(2,6-dioxopiperidin- 3-yl)-1,3- dioxoisoindolin-4- yl)hexyl)piperazin-1- yl)pyridazine-3- carbonyl)piperidin-4- yl)butyl)-3-(pyridin-3- yl)acrylamide |
| | 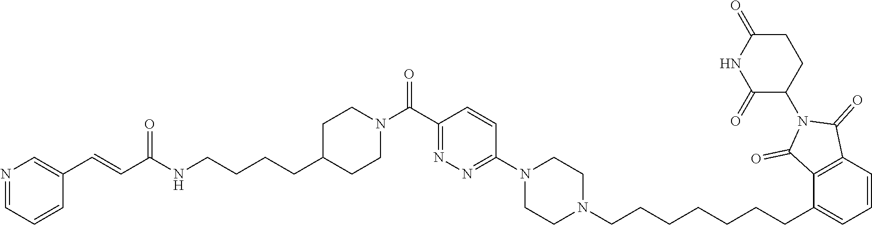 [SMILES: O=C(/C=C/c1cccnc1)NCCCCC1CCN(C(=O)c2ccc(N3CCN(CCCCCCCc4cccc5c4C(=O)N(C4CCC(=O)NC4=O)C5=O)CC3)nn2)CC1] | (E)-N-(4-(1-(6-(4-(7-(2- (2,6-dioxopiperidin-3- yl)-1,3-dioxoisoindolin- 4-yl)heptyl)piperazin-1- yl)pyridazine-3- carbonyl)piperidin-4- yl)butyl)-3-(pyridin-3- yl)acrylamide | (E)-N-(4-(1-(6-(4-(7- (2-(2,6-dioxopiperidin- 3-yl)-1,3- dioxoisoindolin-4- yl)heptyl)piperazin-1- yl)pyridazine-3- carbonyl)piperidin-4- yl)butyl)-3-(pyridin-3- yl)acrylamide |
| | 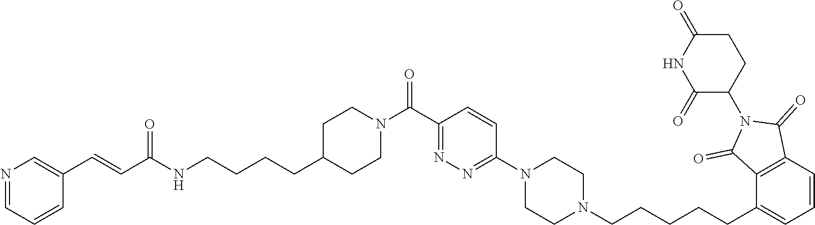 [SMILES: O=C(/C=C/c1cccnc1)NCCCCC1CCN(C(=O)c2ccc(N3CCN(CCCCCc4cccc5c4C(=O)N(C4CCC(=O)NC4=O)C5=O)CC3)nn2)CC1] | (E)-N-(4-(1-(6-(4-(5-(2- (2,6-dioxopiperidin-3- yl)-1,3-dioxoisoindolin- 4-yl)pentyl)piperazin-1- yl)pyridazine-3- carbonyl)piperidin-4- yl)butyl)-3-(pyridin-3- yl)acrylamide | (E)-N-(4-(1-(6-(4-(5- (2-(2,6-dioxopiperidin- 3-yl)-1,3- dioxoisoindolin-4- yl)pentyl)piperazin-1- yl)pyridazine-3- carbonyl)piperidin-4- yl)butyl)-3-(pyridin-3- yl)acrylamide |
| | 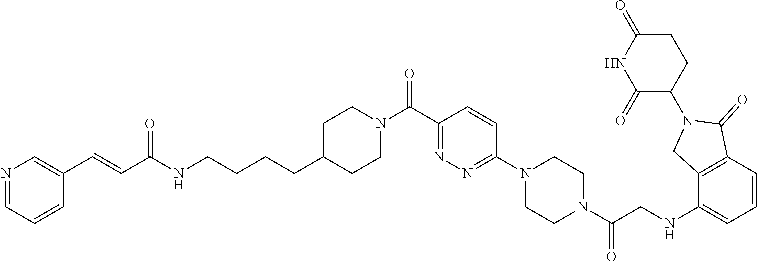 [SMILES: O=C(/C=C/c1cccnc1)NCCCCC1CCN(C(=O)c2ccc(N3CCN(C(=O)CNc4cccc5c4CN(C4CCC(=O)NC4=O)C5=O)CC3)nn2)CC1] | (E)-N-(4-(1-(6-(4-((2- (2,6-dioxopiperidin-3- yl)-1-oxoisoindolin-4- yl)glycyl)piperazin-1- yl)pyridazine-3- carbonyl)piperidin-4- yl)butyl)-3-(pyridin-3- yl)acrylamide | (E)-N-(4-(1-(6-(4-((2- (2,6-dioxopiperidin-3- yl)-1-oxoisoindolin-4- yl)glycyl)piperazin-1- yl)pyridazine-3- carbonyl)piperidin-4- yl)butyl)-3-(pyridin-3- yl)acrylamide |
| | 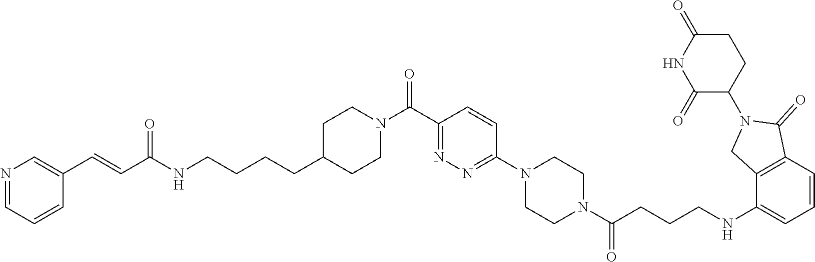 [SMILES: O=C(/C=C/c1cccnc1)NCCCCC1CCN(C(=O)c2ccc(N3CCN(C(=O)CCCNc4cccc5c4CN(C4CCC(=O)NC4=O)C5=O)CC3)nn2)CC1] | (E)-N-(4-(1-(6-(4-(4- ((2-(2,6-dioxopiperidin- 3-yl)-1-oxoisoindolin-4- yl)amino)butanoyl) piperazin-1- yl)pyridazine-3- carbonyl)piperidin-4- yl)butyl)-3-(pyridin-3- yl)acrylamide | (E)-N-(4-(1-(6-(4-(4- ((2-(2,6- dioxopiperidin-3-yl)- 1-oxoisoindolin-4- yl)amino)butanoyl) piperazin-1- yl)pyridazine- 3-carbonyl)piperidin-4- yl)butyl)-3-(pyridin-3- yl)acrylamide |
| | 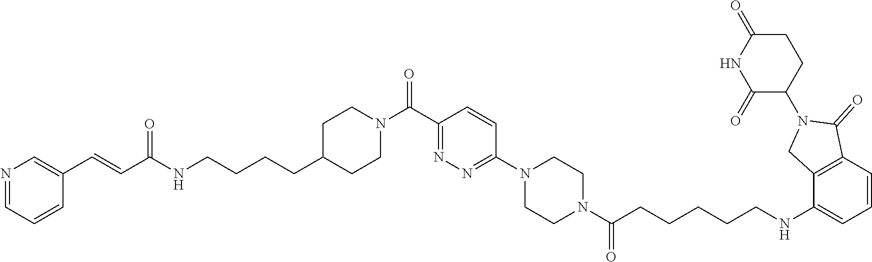 [SMILES: O=C(/C=C/c1cccnc1)NCCCCC1CCN(C(=O)c2ccc(N3CCN(C(=O)CCCCCNc4cccc5c4CN(C4CCC(=O)NC4=O)C5=O)CC3)nn2)CC1] | (E)-N-(4-(1-(6-(4-(6- ((2-(2,6-dioxopiperidin- 3-yl)-1-oxoisoindolin-4- yl)amino)hexanoyl)pipe razin-1-yl)pyridazine-3- carbonyl)piperidin-4- yl)butyl)-3-(pyridin-3- yl)acrylamide | (E)-N-(4-(1-(6-(4-(6- ((2-(2,6- dioxopiperidin-3-yl)-1- oxoisoindolin-4- yl)amino)hexanoyl)pip erazin-1-yl)pyridazine- 3-carbonyl)piperidin-4- yl)butyl)-3-(pyridin-3- yl)acrylamide |
| | 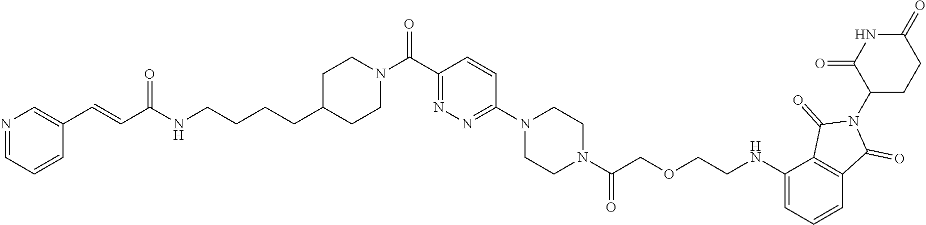 [SMILES: O=C(/C=C/c1cccnc1)NCCCCC1CCN(C(=O)c2ccc(N3CCN(C(=O)COCCNc4cccc5c4C(=O)N(C4CCC(=O)NC4=O)C5=O)CC3)nn2)CC1] | (E)-N-(4-(1-(6-(4-(2-(2- ((2-(2,6-dioxopiperidin- 3-yl)-1-oxoisoindolin-4- yl)amino)ethoxy)acetyl) piperazin-1- yl)pyridazine-3- carbonyl)piperidin-4- yl)butyl)-3-(pyridin-3- yl)acrylamide | (E)-N-(4-(1-(6-(4-(2- (2-((2-(2,6- dioxopiperidin-3-yl)- 1-oxoisoindolin-4- yl)amino)ethoxy) acetyl)piperazin-1- yl)pyridazine-3- carbonyl)piperidin-4- yl)butyl)-3-(pyridin-3- yl)acrylamide |
| | 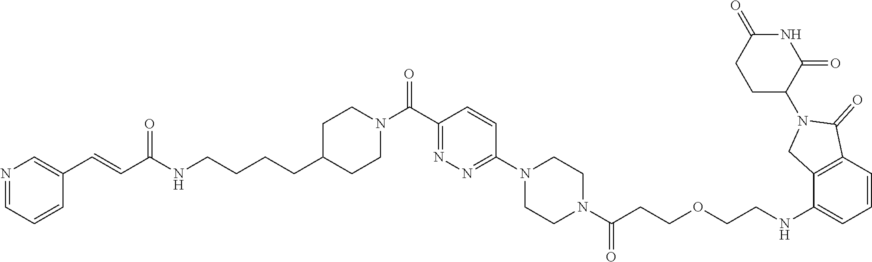 [SMILES: O=C(/C=C/c1cccnc1)NCCCCC1CCN(C(=O)c2ccc(N3CCN(C(=O)CCOCCNc4cccc5c4CN(C4CCC(=O)NC4=O)C5=O)CC3)nn2)CC1] | (E)-N-(4-(1-(6-(4-(3-(2- ((2-(2,6-dioxopiperidin- 3-yl)-1-oxoisoindolin-4- yl)amino)ethoxy) propanoyl)piperazin-1- yl)pyridazine-3- carbonyl)piperidin-4- yl)butyl)-3-(pyridin-3- yl)acrylamide | (E)-N-(4-(1-(6-(4-(3- (2-((2-(2,6- dioxopiperidin-3-yl)- 1-oxoisoindolin-4- yl)amino)ethoxy) propanoyl)piperazin- 1-yl)pyridazine-3- carbonyl)piperidin-4- yl)butyl)-3-(pyridin-3- yl)acrylamide |
| | 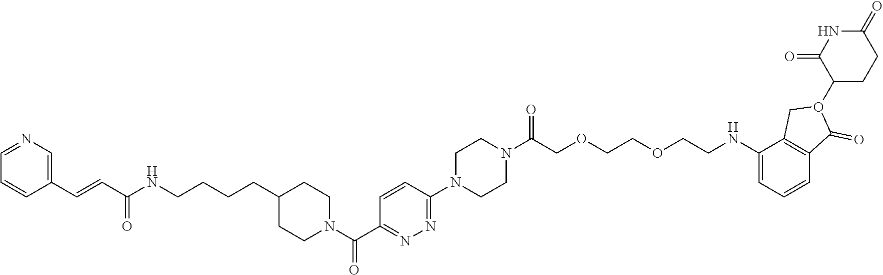 | (E)-N-(4-(1-(6-(4-(2-(2- (2-((2-(2,6- dioxopiperidin-3-yl)- 1-oxoisoindolin-4- yl)amino)ethoxy) ethoxy)acetyl) piperazin-1- yl)pyridazine-3- carbonyl)piperidin-4- yl)butyl)-3-(pyridin-3- yl)acrylamide | (E)-N-(4-(1-(6-(4-(2- (2-(2-((2-(2,6- dioxopiperidin-3-yl)- 1-oxoisoindolin-4- yl)amino)ethoxy) ethoxy)acetyl) piperazin-1- yl)pyridazine-3- carbonyl)piperidin-4- yl)butyl)-3-(pyridin-3- yl)acrylamide |
| | 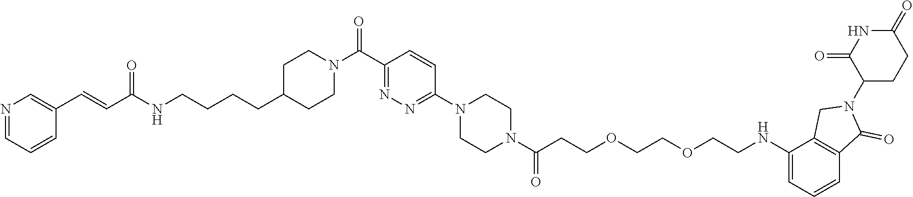 [SMILES: O=C(/C=C/c1cccnc1)NCCCCC1CCN(C(=O)c2ccc(N3CCN(C(=O)CCOCCOCCNc4cccc5c4CN(C4CCC(=O)NC4=O)C5=O)CC3)nn2)CC1] | (E)-N-(4-(1-(6-(4-(3-(2- (2-((2-(2,6- dioxopiperidin-3-yl)- 1-oxoisoindolin-4- yl)amino)ethoxy) ethoxy)propanoyl) piperazin-1- yl)pyridazine-3- carbonyl)piperidin-4- yl)butyl)-3-(pyridin-3- yl)acrylamide | (E)-N-(4-(1-(6-(4-(3- (2-(2-((2-(2,6- dioxopiperidin-3-yl)- 1-oxoisoindolin-4- yl)amino)ethoxy) ethoxy)propanoyl) piperazin- 1-yl)pyridazine-3- carbonyl)piperidin-4- yl)butyl)-3-(pyridin-3- yl)acrylamide |
| | 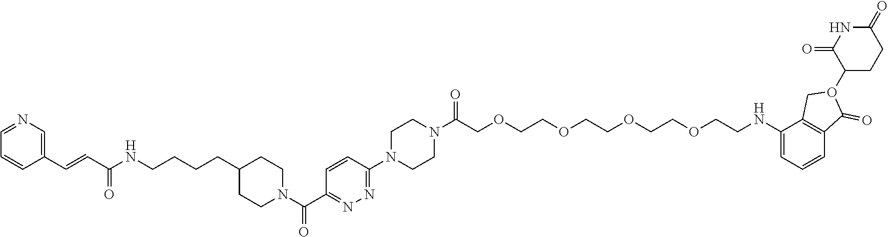 | (E)-N-(4-(1-(6-(4-(14- ((2-(2,6-dioxopiperidin- 3-yl)-1-oxoisoindolin- 4-yl)amino)-3,6,9,12- tetraoxatetradecanoyl) piperazin-1- yl)pyridazine- 3-carbonyl)piperidin-4- yl)butyl)-3-(pyridin-3- yl)acrylamide | (E)-N-(4-(1-(6-(4-(14- ((2-(2,6- dioxopiperidin-3-yl)- 1-oxoisoindolin-4- yl)amino)-3,6,9,12- tetraoxatetradecanoyl) piperazin-1- yl)pyridazine-3- carbonyl)piperidin-4- yl)butyl)-3-(pyridin- 3-yl)acrylamide |
| | 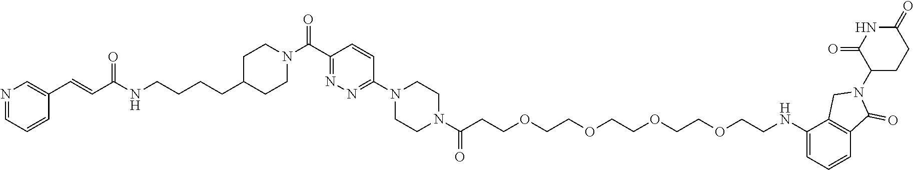 [SMILES: O=C(/C=C/c1cccnc1)NCCCCC1CCN(C(=O)c2ccc(N3CCN(C(=O)CCOCCOCCOCCOCCNc4cccc5c4CN(C4CCC(=O)NC4=O)C5=O)CC3)nn2)CC1] | (E)-N-(4-(1-(6-(4-(1- ((2-(2,6-dioxopiperidin- 3-yl)-1-oxoisoindolin-4- yl)amino)-3,6,9,12- tetraoxapentadecan-15- oyl)piperazin-1- yl)pyridazine-3- carbonyl)piperidin-4- yl)butyl)-3-(pyridin-3- yl)acrylamide | (E)-N-(4-(1-(6-(4-(1- ((2-(2,6- dioxopiperidin-3-yl)- 1-oxoisoindolin-4- yl)amino)-3,6,9,12- tetraoxapentadecan- 15-oyl)piperazin-1- yl)pyridazine-3- carbonyl)piperidin-4- yl)butyl)-3-(pyridin-3- yl)acrylamide |
| | 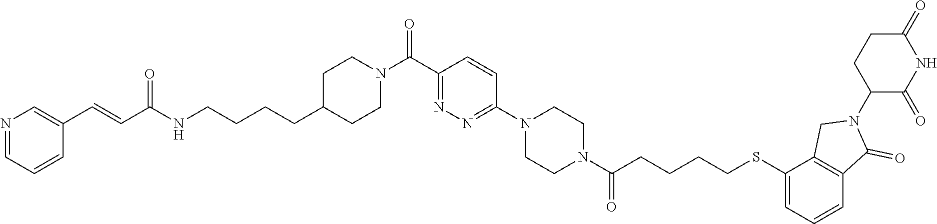 [SMILES: O=C(/C=C/c1cccnc1)NCCCCC1CCN(C(=O)c2ccc(N3CCN(C(=O)CCCCSc4cccc5c4CN(C4CCC(=O)NC4=O)C5=O)CC3)nn2)CC1] | (E)-N-(4-(1-(6-(4-(5- ((2-(2,6-dioxopiperidin- 3-yl)-1-oxoisoindolin- 4-yl)thio)pentanoyl) piperazin-1- yl)pyridazine-3- carbonyl)piperidin-4- yl)butyl)-3-(pyridin-3- yl)acrylamide | (E)-N-(4-(1-(6-(4-(5- ((2-(2,6- dioxopiperidin-3-yl)- 1-oxoisoindolin-4- yl)thio)pentanoyl) piperazin-1- yl)pyridazine-3- carbonyl)piperidin-4- yl)butyl)-3-(pyridin-3- yl)acrylamide |
| | 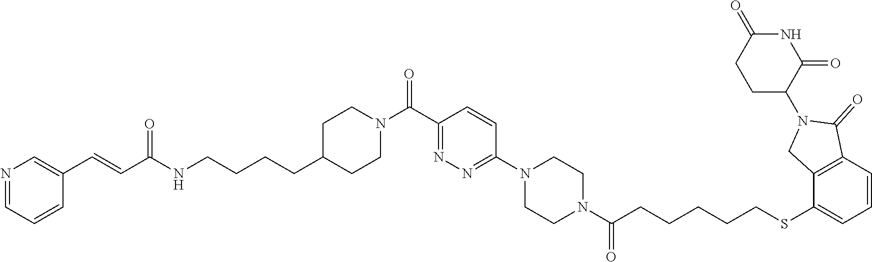 [SMILES: O=C(/C=C/c1cccnc1)NCCCCC1CCN(C(=O)c2ccc(N3CCN(C(=O)CCCCCSc4cccc5c4CN(C4CCC(=O)NC4=O)C5=O)CC3)nn2)CC1] | (E)-N-(4-(1-(6-(4-(6- ((2-(2,6-dioxopiperidin- 3-yl)-1-oxoisoindolin-4- yl)thio)hexanoyl) piperazin-1- yl)pyridazine-3- carbonyl)piperidin-4- yl)butyl)-3-(pyridin-3- yl)acrylamide | (E)-N-(4-(1-(6-(4-(6- ((2-(2,6- dioxopiperidin-3-yl)- 1-oxoisoindolin-4- yl)thio)hexanoyl) piperazin-1- yl)pyridazine-3- carbonyl)piperidin-4- yl)butyl)-3-(pyridin-3- yl)acrylamide |
| | 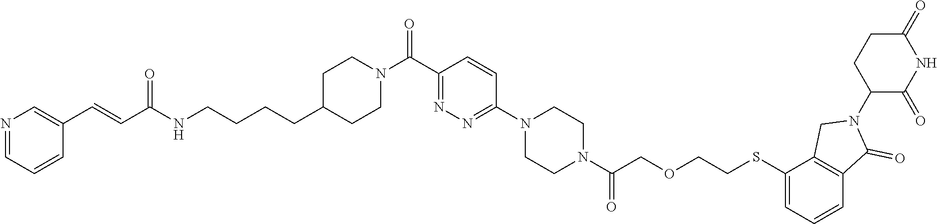 [SMILES: O=C(/C=C/c1cccnc1)NCCCCC1CCN(C(=O)c2ccc(N3CCN(C(=O)COCCSc4cccc5c4CN(C4CCC(=O)NC4=O)C5=O)CC3)nn2)CC1] | (E)-N-(4-(1-(6-(4-(2-(2- ((2-(2,6-dioxopiperidin- 3-yl)-1-oxoisoindolin-4- yl)thio)ethoxy)acetyl) piperazin-1- yl)pyridazine- 3-carbonyl)piperidin-4- yl)butyl)-3-(pyridin-3- yl)acrylamide | (E)-N-(4-(1-(6-(4-(2- (2-((2-(2,6- dioxopiperidin-3-yl)- 1-oxoisoindolin-4- yl)thio)ethoxy)acetyl) piperazin-1- yl)pyridazine-3- carbonyl)piperidin-4- yl)butyl)-3-(pyridin-3- yl)acrylamide |
| | 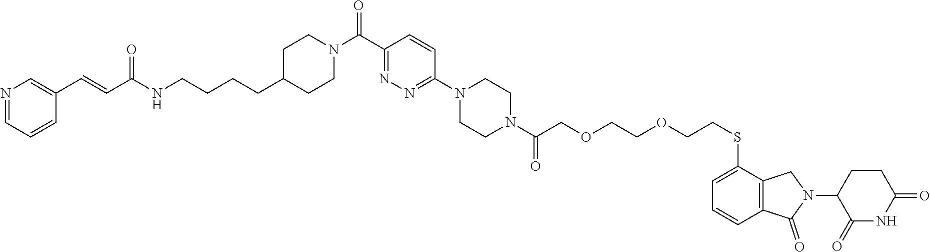 [SMILES: O=C(/C=C/c1cccnc1)NCCCCC1CCN(C(=O)c2ccc(N3CCN(C(=O)COCCOCCSc4cccc5c4CN(C4CCC(=O)NC4=O)C5=O)CC3)nn2)CC1] | (E)-N-(4-(1-(6-(4-(2-(2- (2-((2-(2,6- dioxopiperidin-3-yl)- 1-oxoisoindolin-4- yl)thio)ethoxy)ethoxy) acetyl)piperazin-1- yl)pyridazine-3- carbonyl)piperidin-4- yl)butyl)-3-(pyridin-3- yl)acrylamide | (E)-N-(4-(1-(6-(4-(2- (2-(2-((2-(2,6- dioxopiperidin-3-yl)- 1-oxoisoindolin-4- yl)thio)ethoxy)ethoxy) acetyl)piperazin-1- yl)pyridazine-3- carbonyl)piperidin-4- yl)butyl)-3-(pyridin-3- yl)acrylamide |
| | 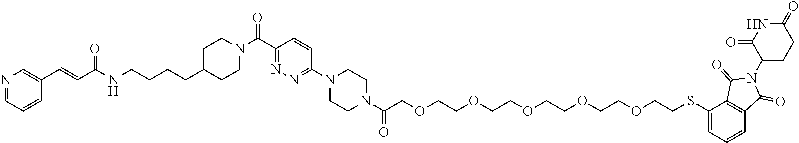 [SMILES: O=C(/C=C/c1cccnc1)NCCCCC1CCN(C(=O)c2ccc(N3CCN(C(=O)COCCOCCOCCOCCOCCSc4cccc5c4C(=O)N(C4CCC(=O)NC4=O)C5=O)CC3)nn2)CC1] | (E)-N-(4-(1-(6-(4-(17- ((2-(2,6-dioxopiperidin- 3-yl)-1-oxoisoindolin-4- yl)thio)-3,6,9,12,15- pentaoxaheptadecanoyl) piperazin-1- yl)pyridazine-3- carbonyl)piperidin-4- yl)butyl)-3-(pyridin-3- yl)acrylamide | (E)-N-(4-(1-(6-(4-(17- ((2-(2,6- dioxopiperidin-3-yl)- 1-oxoisoindolin-4- yl)thio)-3,6,9,12,15- pentaoxaheptadecanoyl) piperazin-1- yl)pyridazine-3- carbonyl)piperidin-4- yl)butyl)-3-(pyridin-3- yl)acrylamide |
| | 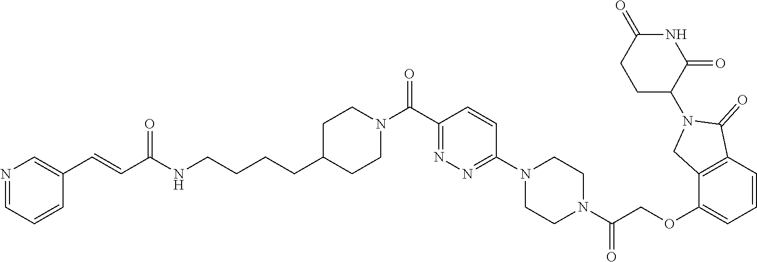 [SMILES: O=C(/C=C/c1cccnc1)NCCCCC1CCN(C(=O)c2ccc(N3CCN(C(=O)COc4cccc5c4CN(C4CCC(=O)NC4=O)C5=O)CC3)nn2)CC1] | (E)-N-(4-(1-(6-(4-(2- ((2-(2,6-dioxopiperidin- 3-yl)-1-oxoisoindolin-4- yl)oxy)acetyl)piperazin- 1-yl)pyridazine-3- carbonyl)piperidin-4- yl)butyl)-3-(pyridin-3- yl)acrylamide | (E)-N-(4-(1-(6-(4-(2- ((2-(2,6- dioxopiperidin-3-yl)- 1-oxoisoindolin-4- yl)oxy)acetyl) piperazin-1- yl)pyridazine-3- carbonyl)piperidin-4- yl)butyl)-3-(pyridin-3- yl)acrylamide |
| | 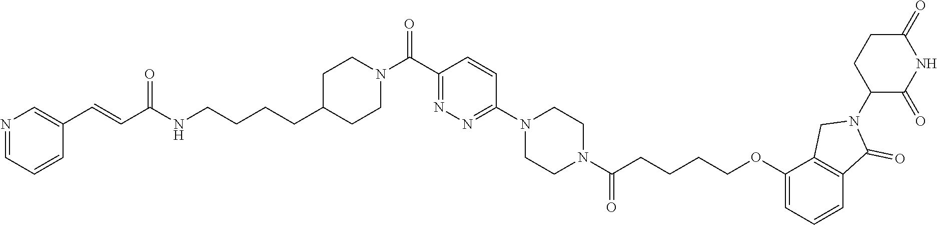 [SMILES: O=C(/C=C/c1cccnc1)NCCCCC1CCN(C(=O)c2ccc(N3CCN(C(=O)CCCCOc4cccc5c4CN(C4CCC(=O)NC4=O)C5=O)CC3)nn2)CC1] | (E)-N-(4-(1-(6-(4-(5- ((2-(2,6-dioxopiperidin- 3-yl)-1-oxoisoindolin- 4-yl)oxy)pentanoyl) piperazin-1- yl)pyridazine-3- carbonyl)piperidin-4- yl)butyl)-3-(pyridin-3- yl)acrylamide | (E)-N-(4-(1-(6-(4-(5- ((2-(2,6- dioxopiperidin-3-yl)- 1-oxoisoindolin-4- yl)oxy)pentanoyl) piperazin-1- yl)pyridazine-3- carbonyl)piperidin-4- yl)butyl)-3-(pyridin-3- yl)acrylamide |
| | 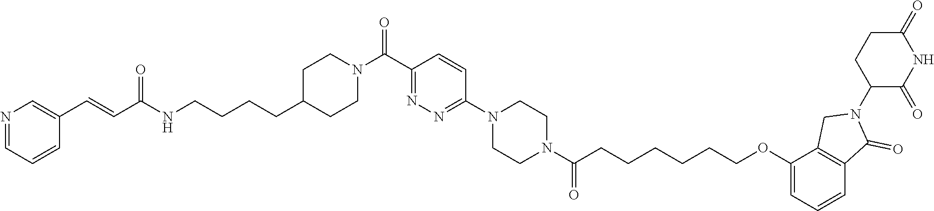 [SMILES: O=C(/C=C/c1cccnc1)NCCCCC1CCN(C(=O)c2ccc(N3CCN(C(=O)CCCCCCOc4cccc5c4CN(C4CCC(=O)NC4=O)C5=O)CC3)nn2)CC1] | (E)-N-(4-(1-(6-(4-(7- ((2-(2,6-dioxopiperidin- 3-yl)-1-oxoisoindolin- 4-yl)oxy)heptanoyl) piperazin-1- yl)pyridazine-3- carbonyl)piperidin-4- yl)butyl)-3-(pyridin-3- yl)acrylamide | (E)-N-(4-(1-(6-(4-(7- ((2-(2,6- dioxopiperidin-3-yl)- 1-oxoisoindolin-4- yl)oxy)heptanoyl) piperazin-1- yl)pyridazine-3- carbonyl)piperidin-4- yl)butyl)-3-(pyridin-3- yl)acrylamide |
| | 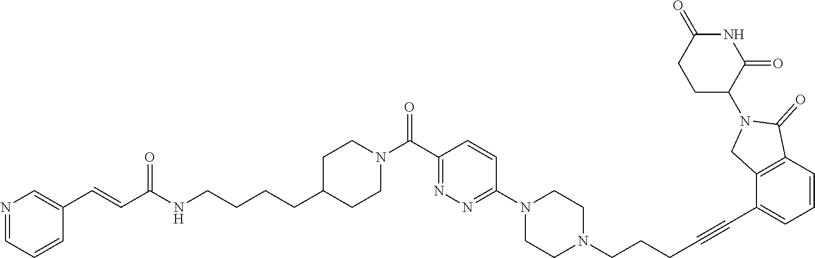 [SMILES: O=C(/C=C/c1cccnc1)NCCCCC1CCN(C(=O)c2ccc(N3CCN(CCCC#Cc4cccc5c4CN(C4CCC(=O)NC4=O)C5=O)CC3)nn2)CC1] | (E)-N-(4-(1-(6-(4-(5-(2- (2,6-dioxopiperidin-3- yl)-1-oxoisoindolin-4- yl)pent-4-yn-1- yl)piperazin-1- yl)pyridazine-3- carbonyl)piperidin-4- yl)butyl)-3-(pyridin-3- yl)acrylamide | (E)-N-(4-(1-(6-(4-(5- (2-(2,6-dioxopiperidin- 3-yl)-1-oxoisoindolin- 4-yl)pent-4-yn-1- yl)piperazin-1- yl)pyridazine-3- carbonyl)piperidin-4- yl)butyl)-3-(pyridin-3- yl)acrylamide |
| | 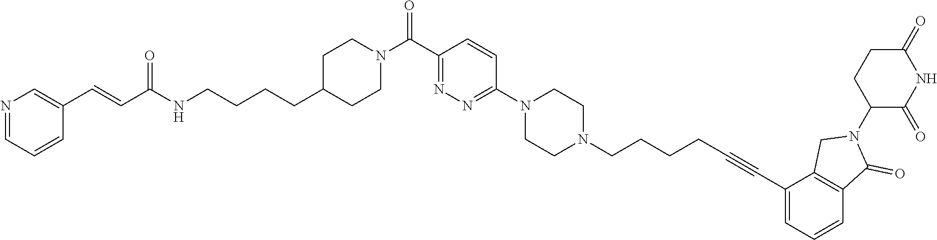 [SMILES: O=C(/C=C/c1cccnc1)NCCCCC1CCN(C(=O)c2ccc(N3CCN(CCCCC#Cc4cccc5c4CN(C4CCC(=O)NC4=O)C5=O)CC3)nn2)CC1] | (E)-N-(4-(1-(6-(4-(6-(2- (2,6-dioxopiperidin-3- yl)-1-oxoisoindolin-4- yl)hex-5-yn-1- yl)piperazin-1- yl)pyridazine-3- carbonyl)piperidin-4- yl)butyl)-3-(pyridin-3- yl)acrylamide | (E)-N-(4-(1-(6-(4-(6- (2-(2,6-dioxopiperidin- 3-yl)-1-oxoisoindolin- 4-yl)hex-5-yn-1- yl)piperazin-1- yl)pyridazine-3- carbonyl)piperidin-4- yl)butyl)-3-(pyridin-3- yl)acrylamide |
| | 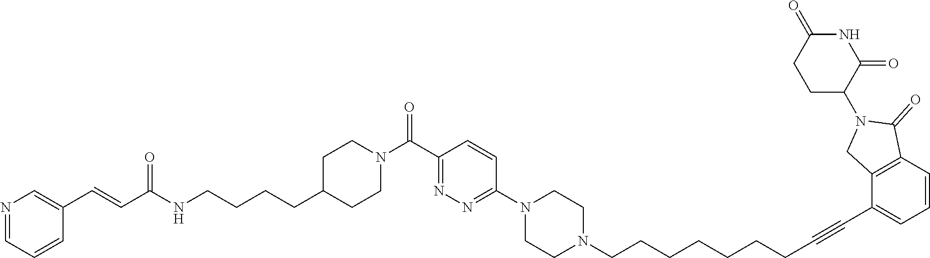 [SMILES: O=C(/C=C/c1cccnc1)NCCCCC1CCN(C(=O)c2ccc(N3CCN(CCCCCCCC#Cc4cccc5c4CN(C4CCC(=O)NC4=O)C5=O)CC3)nn2)CC1] | (E)-N-(4-(1-(6-(4-(9-(2- (2,6-dioxopiperidin-3- yl)-1-oxoisoindolin-4- yl)non-8-yn-1- yl)piperazin-1- yl)pyridazine-3- carbonyl)piperidin-4- yl)butyl)-3-(pyridin-3- yl)acrylamide | (E)-N-(4-(1-(6-(4-(9- (2-(2,6-dioxopiperidin- 3-yl)-1-oxoisoindolin- 4-yl)non-8-yn-1- yl)piperazin-1- yl)pyridazine-3- carbonyl)piperidin-4- yl)butyl)-3-(pyridin-3- yl)acrylamide |
| | 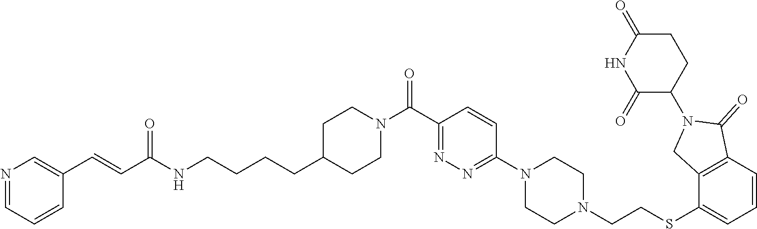 [SMILES: O=C(/C=C/c1cccnc1)NCCCCC1CCN(C(=O)c2ccc(N3CCN(CCSc4cccc5c4CN(C4CCC(=O)NC4=O)C5=O)CC3)nn2)CC1] | (E)-N-(4-(1-(6-(4-(2- ((2-(2,6-dioxopiperidin- 3-yl)-1-oxoisoindolin-4- yl)thio)ethyl)piperazin- 1-yl)pyridazine-3- carbonyl)piperidin-4- yl)butyl)-3-(pyridin-3- yl)acrylamide | (E)-N-(4-(1-(6-(4-(2- ((2-(2,6- dioxopiperidin-3-yl)- 1-oxoisoindolin-4- yl)thio)ethyl)piperazin- 1-yl)pyridazine-3- carbonyl)piperidin-4- yl)butyl)-3-(pyridin-3- yl)acrylamide |
| | 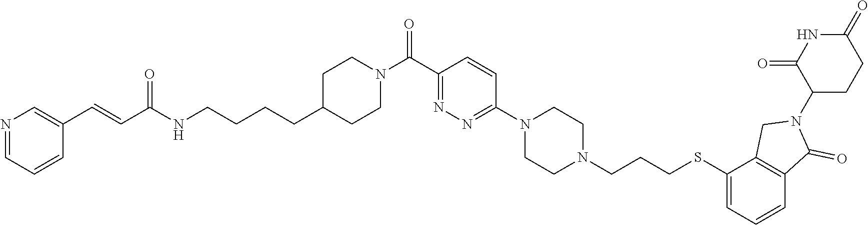 [SMILES: O=C(/C=C/c1cccnc1)NCCCCC1CCN(C(=O)c2ccc(N3CCN(CCCSc4cccc5c4CN(C4CCC(=O)NC4=O)C5=O)CC3)nn2)CC1] | (E)-N-(4-(1-(6-(4-(3- ((2-(2,6-dioxopiperidin- 3-yl)-1-oxoisoindolin-4- yl)thio)propyl) piperazin-1- yl)pyridazine-3- carbonyl)piperidin-4- yl)butyl)-3-(pyridin-3- yl)acrylamide | (E)-N-(4-(1-(6-(4-(3- ((2-(2,6- dioxopiperidin-3-yl)- 1-oxoisoindolin-4- yl)thio)propyl) piperazin-1- yl)pyridazine-3- carbonyl)piperidin-4- yl)butyl)-3-(pyridin-3- yl)acrylamide |
| | 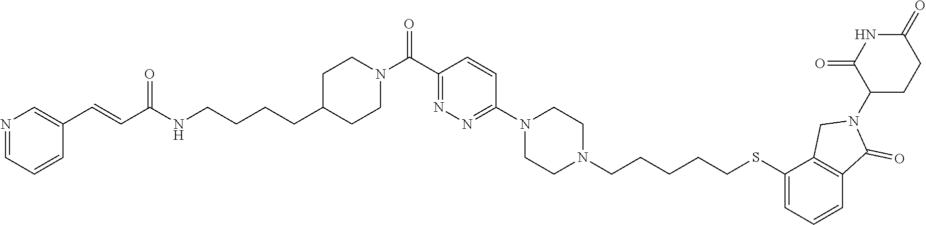 [SMILES: O=C(/C=C/c1cccnc1)NCCCCC1CCN(C(=O)c2ccc(N3CCN(CCCCCSc4cccc5c4CN(C4CCC(=O)NC4=O)C5=O)CC3)nn2)CC1] | (E)-N-(4-(1-(6-(4-(5- ((2-(2,6-dioxopiperidin- 3-yl)-1-oxoisoindolin-4- yl)thio)pentyl)piperazin- 1-yl)pyridazine-3- carbonyl)piperidin-4- yl)butyl)-3-(pyridin-3- yl)acrylamide | (E)-N-(4-(1-(6-(4-(5- ((2-(2,6- dioxopiperidin-3-yl)- 1-oxoisoindolin-4- yl)thio)pentyl) piperazin- 1-yl)pyridazine-3- carbonyl)piperidin-4- yl)butyl)-3-(pyridin-3- yl)acrylamide |
| | 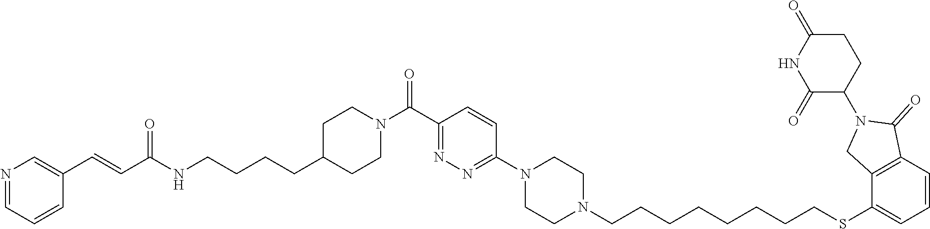 [SMILES: O=C(/C=C/c1cccnc1)NCCCCC1CCN(C(=O)c2ccc(N3CCN(CCCCCCCCSc4cccc5c4CN(C4CCC(=O)NC4=O)C5=O)CC3)nn2)CC1] | (E)-N-(4-(1-(6-(4-(8- ((2-(2,6-dioxopiperidin- 3-yl)-1-oxoisoindolin-4- yl)thio)octyl)piperazin- 1-yl)pyridazine-3- carbonyl)piperidin-4- yl)butyl)-3-(pyridin-3- yl)acrylamide | (E)-N-(4-(1-(6-(4-(8- ((2-(2,6- dioxopiperidin-3-yl)- 1-oxoisoindolin-4- yl)thio)octyl)piperazin- 1-yl)pyridazine-3- carbonyl)piperidin-4- yl)butyl)-3-(pyridin-3- yl)acrylamide |
| | 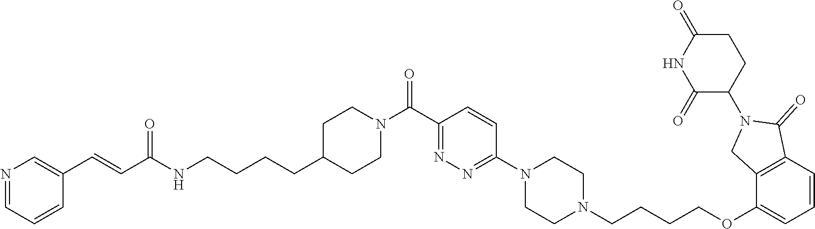 [SMILES: O=C(/C=C/c1cccnc1)NCCCCC1CCN(C(=O)c2ccc(N3CCN(CCCCOc4cccc5c4CN(C4CCC(=O)NC4=O)C5=O)CC3)nn2)CC1] | (E)-N-(4-(1-(6-(4-(4- ((2-(2,6-dioxopiperidin- 3-yl)-1-oxoisoindolin-4- yl)oxy)butyl)piperazin- 1-yl)pyridazine-3- carbonyl)piperidin-4- yl)butyl)-3-(pyridin-3- yl)acrylamide | (E)-N-(4-(1-(6-(4-(4- ((2-(2,6- dioxopiperidin-3-yl)- 1-oxoisoindolin-4- yl)oxy)butyl)piperazin- 1-yl)pyridazine-3- carbonyl)piperidin-4- yl)butyl)-3-(pyridin-3- yl)acrylamide |
| | 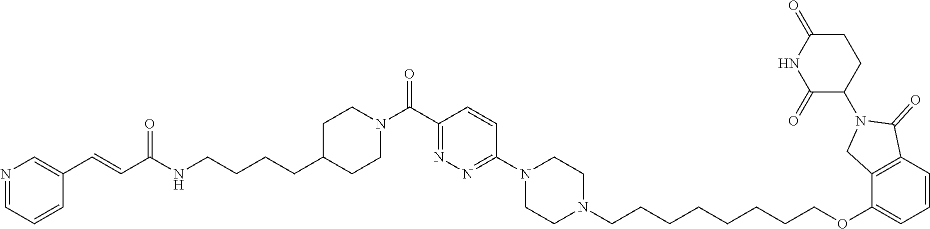 [SMILES: O=C(/C=C/c1cccnc1)NCCCCC1CCN(C(=O)c2ccc(N3CCN(CCCCCCCCOc4cccc5c4CN(C4CCC(=O)NC4=O)C5=O)CC3)nn2)CC1] | (E)-N-(4-(1-(6-(4-(8- ((2-(2,6-dioxopiperidin- 3-yl)-1-oxoisoindolin-4- yl)oxy)octyl)piperazin- 1-yl)pyridazine-3- carbonyl)piperidin-4- yl)butyl)-3-(pyridin-3- yl)acrylamide | (E)-N-(4-(1-(6-(4-(8- ((2-(2,6- dioxopiperidin-3-yl)- 1-oxoisoindolin-4- yl)oxy)octyl)piperazin- 1-yl)pyridazine-3- carbonyl)piperidin-4- yl)butyl)-3-(pyridin-3- yl)acrylamide |
| | 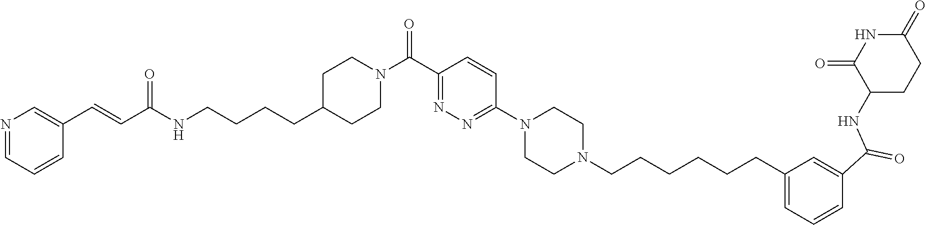 [SMILES: O=C(/C=C/c1cccnc1)NCCCCC1CCN(C(=O)c2ccc(N3CCN(CCCCCCc4cccc(C(=O)NC5CCC(=O)NC5=O)c4)CC3)nn2)CC1] | (E)-N-(2,6- dioxopiperidin-3-yl)-3- (6-(4-(6-(4-(4-(3- (pyridin-3- yl)acrylamido)butyl) piperidine-1- carbonyl)pyridazin-3- yl)piperazin-1- yl)hexyl)benzamide | (E)-N-(2,6- dioxopiperidin-3-yl)- 3-(6-(4-(6-(4-(4-(3- (pyridin-3- yl)acrylamido)butyl) piperidine-1- carbonyl)pyridazin-3- yl)piperazin-1- yl)hexyl)benzamide |
| | 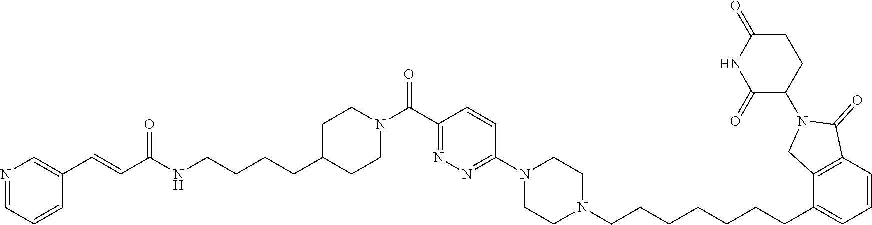 [SMILES: O=C(/C=C/c1cccnc1)NCCCCC1CCN(C(=O)c2ccc(N3CCN(CCCCCCCc4cccc5c4CN(C4CCC(=O)NC4=O)C5=O)CC3)nn2)CC1] | (E)-N-(4-(1-(6-(4-(7-(2- (2,6-dioxopiperidin-3- yl)-1-oxoisoindolin-4- yl)heptyl)piperazin-1- yl)pyridazine-3- carbonyl)piperidin-4- yl)butyl)-3-(pyridin-3- yl)acrylamide | (E)-N-(4-(1-(6-(4-(7- (2-(2,6-dioxopiperidin- 3-yl)-1-oxoisoindolin- 4-yl)heptyl)piperazin- 1-yl)pyridazine-3- carbonyl)piperidin-4- yl)butyl)-3-(pyridin-3- yl)acrylamide |
| | 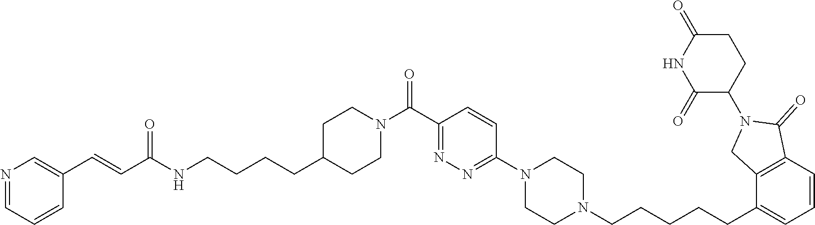 [SMILES: O=C(/C=C/c1cccnc1)NCCCCC1CCN(C(=O)c2ccc(N3CCN(CCCCCc4cccc5c4CN(C4CCC(=O)NC4=O)C5=O)CC3)nn2)CC1] | (E)-N-(4-(1-(6-(4-(5-(2- (2,6-dioxopiperidin-3- yl)-1-oxoisoindolin-4- yl)pentyl)piperazin-1- yl)pyridazine-3- carbonyl)piperidin-4- yl)butyl)-3-(pyridin-3- yl)acrylamide | (E)-N-(4-(1-(6-(4-(5- (2-(2,6-dioxopiperidin- 3-yl)-1-oxoisoindolin- 4-yl)pentyl)piperazin- 1-yl)pyridazine-3- carbonyl)piperidin-4- yl)butyl)-3-(pyridin-3- yl)acrylamide |
| | 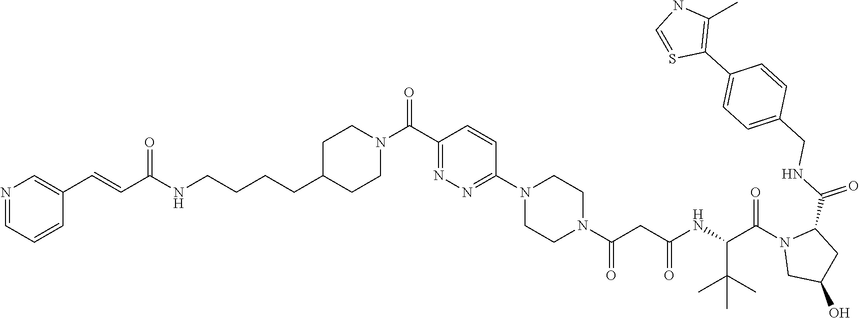 | (2S,4R)-1-((S)-3,3- dimethyl-2-(3-oxo-3-(4- (6-(4-(4-((E)-3-(pyridin- 3-yl)acrylamido)butyl) piperidine-1- carbonyl)pyridazin-3- yl)piperazin-1- yl)propanamido)butanoy 1)-4-hydroxy-N-(4-(4- methylthiazol-5- yl)benzyl)pyrrolidine-2- carboxamide | (2S,4R)-1-((S)-3,3- dimethyl-2-(3-oxo-3- (4-(6-(4-(4-((E)-3- (pyridin-3- yl)acrylamido)butyl) piperidine-1- carbonyl)pyridazin-3- yl)piperazin-1- yl)propanamido)butano yl)-4-hydroxy-N-(4-(4- methylthiazol-5- yl)benzyl)pyrrolidine- 2-carboxamide |
| | 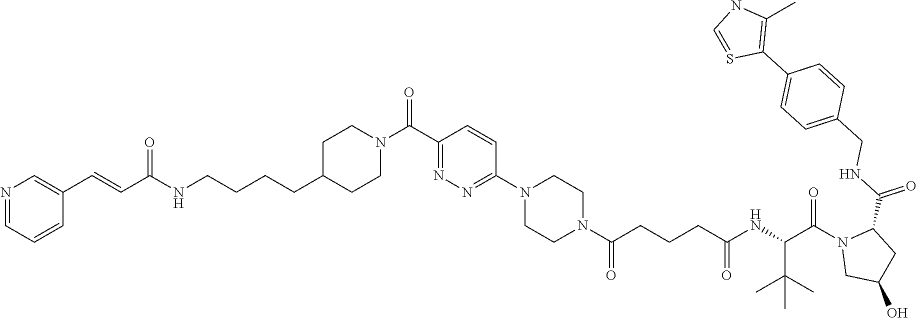 | (2S,4R)-1-((S)-3,3- dimethyl-2-(5-oxo-5-(4- (6-(4-(4-((E)-3-(pyridin- 3-yl)acrylamido)butyl) piperidine-1- carbonyl)pyridazin-3- yl)piperazin-1- yl)pentanamido)butanoy 1)-4-hydroxy-N-(4-(4- methylthiazol-5- yl)benzyl)pyrrolidine-2- carboxamide | (2S,4R)-1-((S)-3,3- dimethyl-2-(5-oxo-5- (4-(6-(4-(4-((E)-3- (pyridin-3- yl)acrylamido)butyl) piperidine-1- carbonyl)pyridazin-3- yl)piperazin-1- yl)pentanamido)butano yl)-4-hydroxy-N-(4-(4- methylthiazol-5- yl)benzyl)pyrrolidine- 2-carboxamide |
| | 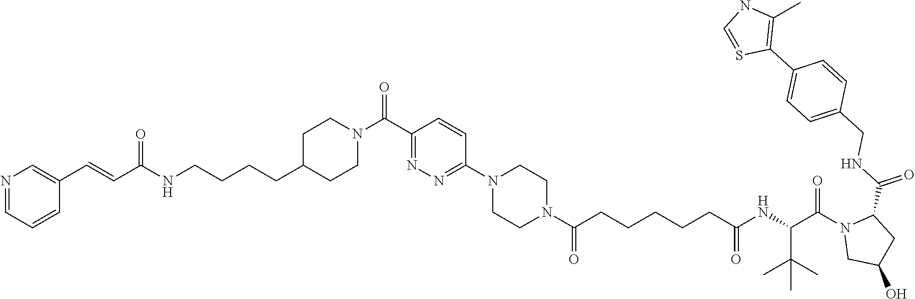 | (2S,4R)-1-((S)-3,3- dimethyl-2-(7-oxo-7-(4- (6-(4-(4-((E)-3-(pyridin- 3-yl)acrylamido)butyl) piperidine-1- carbonyl)pyridazin-3- yl)piperazin-1- yl)heptanamido)butanoy 1)-4-hydroxy-N-(4-(4- methylthiazol-5- yl)benzyl)pyrrolidine-2- carboxamide | (2S,4R)-1-((S)-3,3- dimethyl-2-(7-oxo-7- (4-(6-(4-(4-((E)-3- (pyridin-3- yl)acrylamido)butyl) piperidine-1- carbonyl)pyridazin-3- yl)piperazin-1- yl)heptanamido)butano yl)-4-hydroxy-N-(4-(4- methylthiazol-5- yl)benzyl)pyrrolidine- 2-carboxamide |
| | 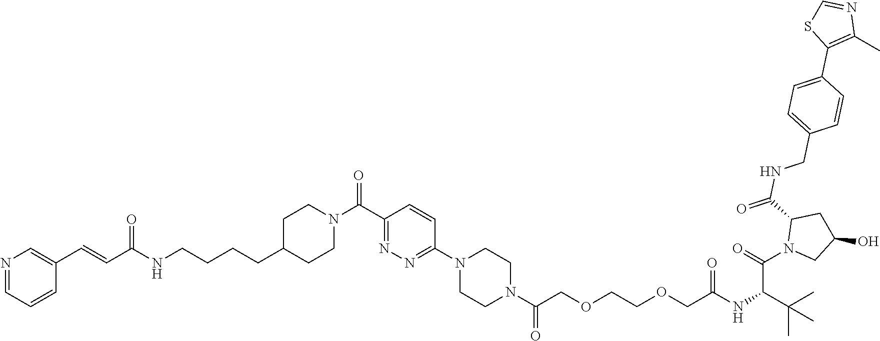 [SMILES: Cc1ncsc1-c1ccc(CNC(=O)[C@@H]2C[C@@H](O)CN2C(=O)[C@@H](NC(=O)COCCOCC(=O)N2CCN(c3ccc(C(=O)N4CCC(CCCCNC(=O)/C=C/c5cccnc5)CC4)nn3)CC2)C(C)(C)C)cc1] | (2S,4R)-1-((S)-3,3- dimethyl-2-(2-(2-(2- oxo-2-(4-(6-(4-(4-((E)- 3-(pyridin-3- yl)acrylamido)butyl) piperidine-1- carbonyl)pyridazin-3- yl)piperazin-1- yl)ethoxy)ethoxy) acetamido)butanoyl)- 4-hydroxy-N-(4-(4- methylthiazol-5- yl)benzyl)pyrrolidine-2- carboxamide | (2S,4R)-1-((S)-3,3- dimethyl-2-(2-(2-(2- oxo-2-(4-(6-(4-(4-((E)- 3-(pyridin-3- yl)acrylamido)butyl) piperidine-1- carbonyl)pyridazin-3- yl)piperazin-1- yl)ethoxy)ethoxy) acetamido)butanoyl)- 4-hydroxy-N-(4-(4- methylthiazol-5- yl)benzyl)pyrrolidine- 2-carboxamide |
| | 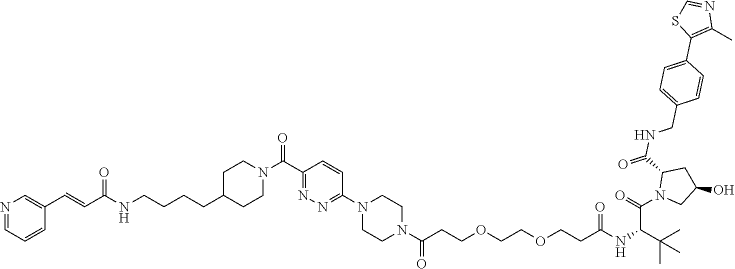 [SMILES: Cc1ncsc1-c1ccc(CNC(=O)[C@@H]2C[C@@H](O)CN2C(=O)[C@@H](NC(=O)CCOCCOCCC(=O)N2CCN(c3ccc(C(=O)N4CCC(CCCCNC(=O)/C=C/c5cccnc5)CC4)nn3)CC2)C(C)(C)C)cc1] | (2S,4R)-1-((S)-3,3- dimethyl-2-(3-(2-(3- ox0-3-(4-(6-(4-(4-((E)- 3-(pyridin-3- yl)acrylamido)butyl) piperidine-1- carbonyl)pyridazin-3- yl)piperazin-1- yl)propoxy)ethoxy) propanamido)butanoyl)- 4-hydroxy-N-(4-(4- methylthiazol-5- yl)benzyl)pyrrolidine-2- carboxamide | (2S,4R)-1-((S)-3,3- dimethyl-2-(3-(2-(3- oxo-3-(4-(6-(4-(4-((E)- 3-(pyridin-3- yl)acrylamido)butyl) piperidine-1- carbonyl)pyridazin-3- yl)piperazin-1- yl)propoxy)ethoxy) propanamido) butanoyl)-4- hydroxy-N-(4-(4- methylthiazol-5- yl)benzyl)pyrrolidine- 2-carboxamide |
| | 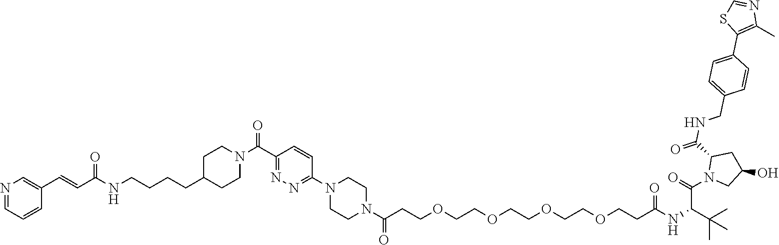 [SMILES: Cc1ncsc1-c1ccc(CNC(=O)[C@@H]2C[C@@H](O)CN2C(=O)[C@@H](NC(=O)CCOCCOCCOCCOCCC(=O)N2CCN(c3ccc(C(=O)N4CCC(CCCCNC(=O)/C=C/c5cccnc5)CC4)nn3)CC2)C(C)(C)C)cc1] | (2S,4R)-1-((S)-2-(tert- butyl)-4,19-dioxo-19-(4- (6-(4-(4-((E)-3-(pyridin- 3-yl)acrylamido)butyl) piperidine-1- carbonyl)pyridazin-3- yl)piperazin-1-yl)- 7,10,13,16-tetraoxa-3- azanonadecanoyl)-4- hydroxy-N-(4-(4- methylthiazol-5- yl)benzyl)pyrrolidine-2- carboxamide | (2S,4R)-1-((S)-2-(tert- butyl)-4,19-dioxo-19- (4-(6-(4-(4-((E)-3- (pyridin-3- yl)acrylamido)butyl) piperidine-1- carbonyl)pyridazin-3- yl)piperazin-1-yl)- 7,10,13,16-tetraoxa-3- azanonadecanoyl)-4- hydroxy-N-(4-(4- methylthiazol-5- yl)benzyl)pyrrolidine- 2-carboxamide |
| | 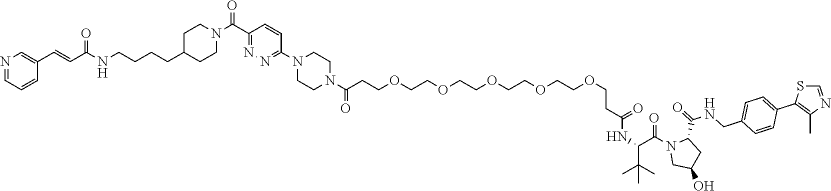 [SMILES: Cc1ncsc1-c1ccc(CNC(=O)[C@@H]2C[C@@H](O)CN2C(=O)[C@@H](NC(=O)CCOCCOCCOCCOCCOCCC(=O)N2CCN(c3ccc(C(=O)N4CCC(CCCCNC(=O)/C=C/c5cccnc5)CC4)nn3)CC2)C(C)(C)C)cc1] | (2S,4R)-1-((S)-2-(tert- butyl)-4,22-dioxo-22-(4- (6-(4-(4-((E)-3-(pyridin- 3-yl)acrylamido)butyl) piperidine-1- carbonyl)pyridazin-3- yl)piperazin-1-yl)- 7,10,13,16,19-pentaoxa- 3-azadocosanoyl)-4- hydroxy-N-(4-(4- methylthiazol-5- yl)benzyl)pyrrolidine-2- carboxamide | (2S,4R)-1-((S)-2-(tert- butyl)-4,22-dioxo-22- (4-(6-(4-(4-((E)-3- (pyridin-3- yl)acrylamido)butyl) piperidine-1- carbonyl)pyridazin-3- yl)piperazin-1-yl)- 7,10,13,16,19- pentaoxa-3- azadocosanoyl)-4- hydroxy-N-(4-(4- methylthiazol-5- |
| | | | yl)benzyl)pyrrolidine- |
| | | | 2-carboxamide |
| | 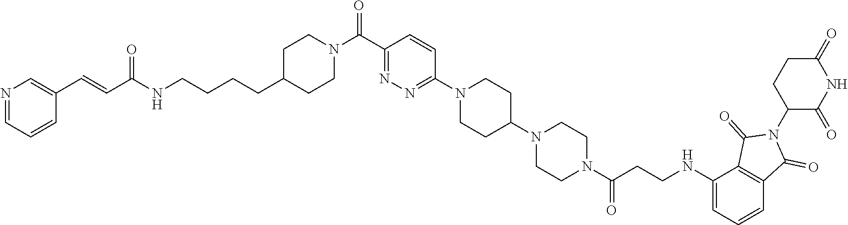 [SMILES: O=C(/C=C/c1cccnc1)NCCCCC1CCN(C(=O)c2ccc(N3CCC(N4CCN(C(=O)CCNc5cccc6c5C(=O)N(C5CCC(=O)NC5=O)C6=O)CC4)CC3)nn2)CC1] | (E)-N-(4-(1-(6-(4-(4-(3- ((2-(2,6-dioxopiperidin- 3-yl)-1,3- dioxoisoindolin-4- yl)amino)propanoyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (3-((2-(2,6- dioxopiperidin-3-yl)- 1,3-dioxoisoindolin-4- yl)amino)propanoyl) piperazin-1- yl)piperidin- 1-yl)pyridazine-3- carbonyl)piperidin-4- yl)butyl)-3-(pyridin-3- yl)acrylamide |
| | 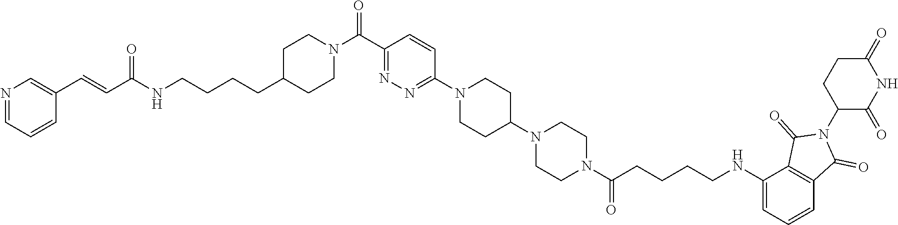 [SMILES: O=C(/C=C/c1cccnc1)NCCCCC1CCN(C(=O)c2ccc(N3CCC(N4CCN(C(=O)CCCCNc5cccc6c5C(=O)N(C5CCC(=O)NC5=O)C6=O)CC4)CC3)nn2)CC1] | (E)-N-(4-(1-(6-(4-(4-(5- ((2-(2,6-dioxopiperidin- 3-yl)-1,3- dioxoisoindolin-4- yl)amino)pentanoyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (5-((2-(2,6- dioxopiperidin-3-yl)- 1,3-dioxoisoindolin-4- yl)amino)pentanoyl) piperazin-1- yl)piperidin-1- yl)pyridazine-3- carbonyl)piperidin-4- yl)butyl)-3-(pyridin-3- yl)acrylamide |
| | 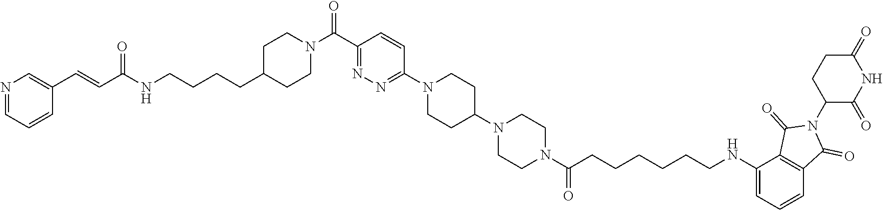 [SMILES: O=C(/C=C/c1cccnc1)NCCCCC1CCN(C(=O)c2ccc(N3CCC(N4CCN(C(=O)CCCCCCNc5cccc6c5C(=O)N(C5CCC(=O)NC5=O)C6=O)CC4)CC3)nn2)CC1] | (E)-N-(4-(1-(6-(4-(4-(7- ((2-(2,6-dioxopiperidin- 3-yl)-1,3- dioxoisoindolin-4- yl)amino)heptanoyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (7-((2-(2,6- dioxopiperidin-3-yl)- 1,3-dioxoisoindolin-4- yl)amino)heptanoyl) piperazin-1- yl)piperidin-1- yl)pyridazine-3- carbonyl)piperidin-4- yl)butyl)-3-(pyridin-3- yl)acrylamide |
| | 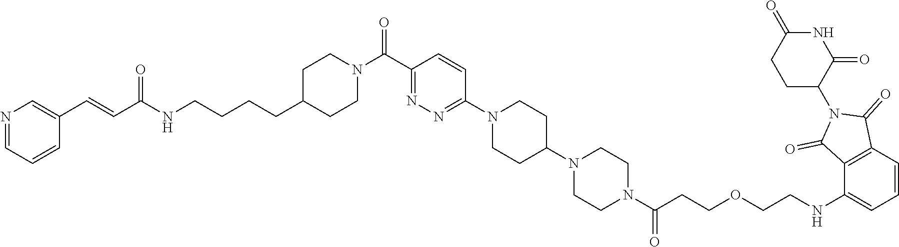 [SMILES: O=C(/C=C/c1cccnc1)NCCCCC1CCN(C(=O)c2ccc(N3CCC(N4CCN(C(=O)CCOCCNc5cccc6c5C(=O)N(C5CCC(=O)NC5=O)C6=O)CC4)CC3)nn2)CC1] | (E)-N-(4-(1-(6-(4-(4-(3- (2-((2-(2,6- dioxopiperidin-3-yl)- 1,3-dioxoisoindolin-4- yl)amino)ethoxy) propanoyl)piperazin- 1-yl)piperidin-1- yl)pyridazine-3- carbonyl)piperidin-4- yl)butyl)-3-(pyridin-3- yl)acrylamide | (E)-N-(4-(1-(6-(4-(4- (3-(2-((2-(2,6- dioxopiperidin-3-yl)- 1,3-dioxoisoindolin-4- yl)amino)ethoxy) propanoyl)piperazin- 1-yl)piperidin-1- yl)pyridazine-3- carbonyl)piperidin-4- yl)butyl)-3-(pyridin-3- yl)acrylamide |
| | 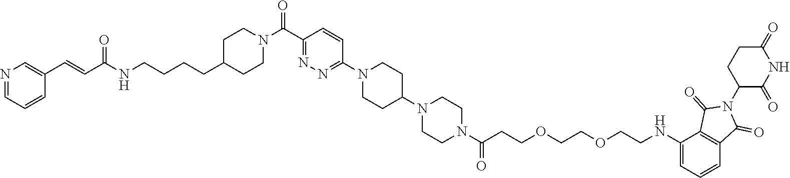 [SMILES: O=C(/C=C/c1cccnc1)NCCCCC1CCN(C(=O)c2ccc(N3CCC(N4CCN(C(=O)CCOCCOCCNc5cccc6c5C(=O)N(C5CCC(=O)NC5=O)C6=O)CC4)CC3)nn2)CC1] | (E)-N-(4-(1-(6-(4-(4-(3- (2-(2-((2-(2,6- dioxopiperidin-3-yl)- 1,3-dioxoisoindolin-4- yl)amino)ethoxy) ethoxy)propanoyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (3-(2-(2-((2-(2,6- dioxopiperidin-3-yl)- 1,3-dioxoisoindolin-4- yl)amino)ethoxy) ethoxy)propanoyl) piperazin- 1-yl)piperidin-1- yl)pyridazine-3- carbonyl)piperidin-4- yl)butyl)-3-(pyridin-3- yl)acrylamide |
| | 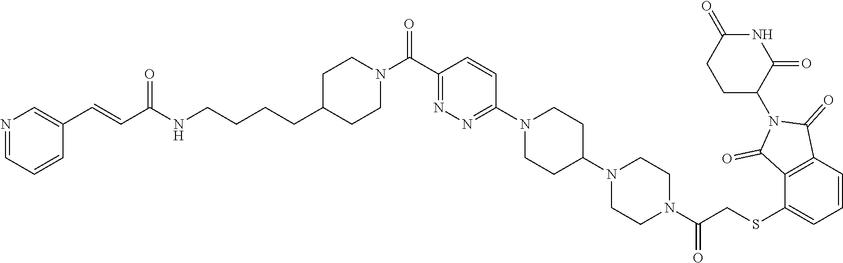 [SMILES: O=C(/C=C/c1cccnc1)NCCCCC1CCN(C(=O)c2ccc(N3CCC(N4CCN(C(=O)CSc5cccc6c5C(=O)N(C5CCC(=O)NC5=O)C6=O)CC4)CC3)nn2)CC1] | (E)-N-(4-(1-(6-(4-(4-(2- ((2-(2,6-dioxopiperidin- 3-yl)-1,3- dioxoisoindolin-4- yl)thio)acetyl) piperazin- 1-yl)piperidin-1- yl)pyridazine-3- carbonyl)piperidin-4- yl)butyl)-3-(pyridin-3- yl)acrylamide | (E)-N-(4-(1-(6-(4-(4- (2-((2-(2,6- dioxopiperidin-3-yl)- 1,3-dioxoisoindolin- 4-yl)thio)acetyl) piperazin-1- yl)piperidin-1- yl)pyridazine-3- carbonyl)piperidin-4- yl)butyl)-3-(pyridin-3- yl)acrylamide |
| | 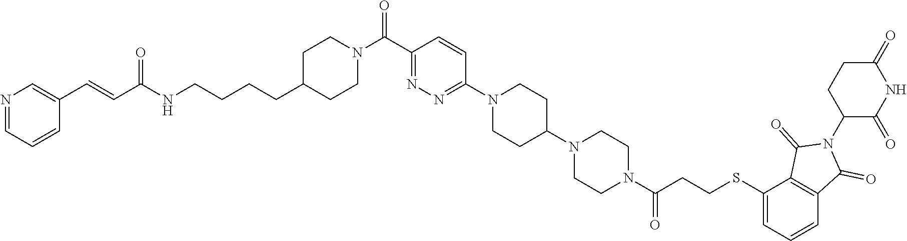 [SMILES: O=C(/C=C/c1cccnc1)NCCCCC1CCN(C(=O)c2ccc(N3CCC(N4CCN(C(=O)CCSc5cccc6c5C(=O)N(C5CCC(=O)NC5=O)C6=O)CC4)CC3)nn2)CC1] | (E)-N-(4-(1-(6-(4-(4-(3- ((2-(2,6-dioxopiperidin- 3-yl)-1,3- dioxoisoindolin-4- yl)thio)propanoyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (3-((2-(2,6- dioxopiperidin-3-yl)- 1,3-dioxoisoindolin-4- yl)thio)propanoyl) piperazin-1- yl)piperidin-1- yl)pyridazine-3- carbonyl)piperidin-4- yl)butyl)-3-(pyridin-3- yl)acrylamide |
| | 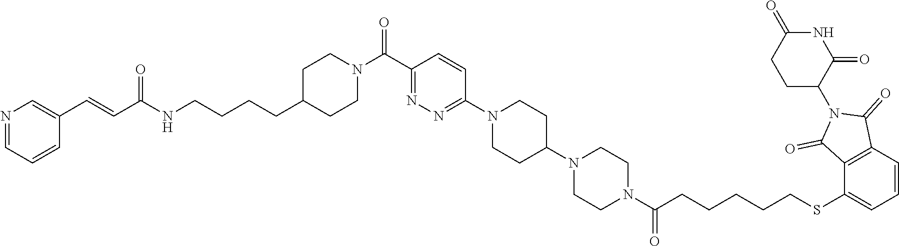 [SMILES: O=C(/C=C/c1cccnc1)NCCCCC1CCN(C(=O)c2ccc(N3CCC(N4CCN(C(=O)CCCCCSc5cccc6c5C(=O)N(C5CCC(=O)NC5=O)C6=O)CC4)CC3)nn2)CC1] | (E)-N-(4-(1-(6-(4-(4-(6- ((2-(2,6-dioxopiperidin- 3-yl)-1,3- dioxoisoindolin-4- yl)thio)hexanoyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (6-((2-(2,6- dioxopiperidin-3-yl)- 1,3-dioxoisoindolin-4- yl)thio)hexanoyl) piperazin-1- yl)piperidin-1- yl)pyridazine-3- carbonyl)piperidin-4- yl)butyl)-3-(pyridin-3- yl)acrylamide |
| | 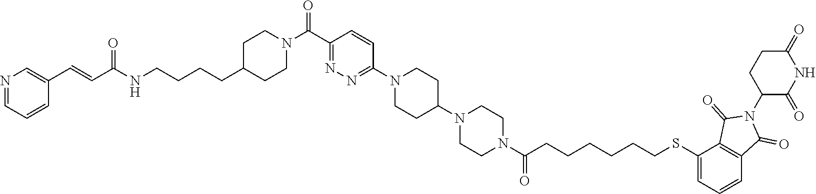 [SMILES: O=C(/C=C/c1cccnc1)NCCCCC1CCN(C(=O)c2ccc(N3CCC(N4CCN(C(=O)CCCCCCSc5cccc6c5C(=O)N(C5CCC(=O)NC5=O)C6=O)CC4)CC3)nn2)CC1] | (E)-N-(4-(1-(6-(4-(4-(7- ((2-(2,6-dioxopiperidin- 3-yl)-1,3- dioxoisoindolin-4- yl)thio)heptanoyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (7-((2-(2,6- dioxopiperidin-3-yl)- 1,3-dioxoisoindolin-4- yl)thio)heptanoyl) piperazin-1- yl)piperidin-1- yl)pyridazine-3- carbonyl)piperidin-4- yl)butyl)-3-(pyridin-3- yl)acrylamide |
| | 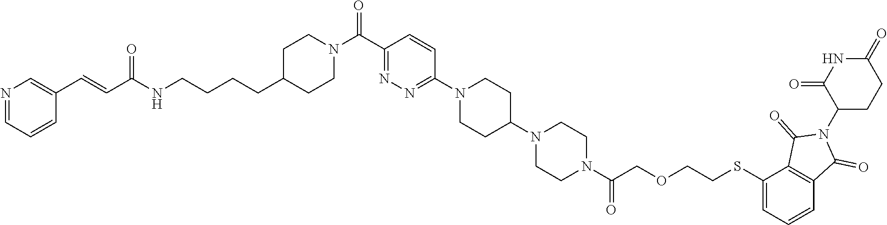 [SMILES: O=C(/C=C/c1cccnc1)NCCCCC1CCN(C(=O)c2ccc(N3CCC(N4CCN(C(=O)COCCSc5cccc6c5C(=O)N(C5CCC(=O)NC5=O)C6=O)CC4)CC3)nn2)CC1] | (E)-N-(4-(1-(6-(4-(4-(2- (2-((2-(2,6- dioxopiperidin-3-yl)- 1,3-dioxoisoindolin-4- yl)thio)ethoxy)acetyl) piperazin-1- yl)piperidin- 1-yl)pyridazine-3- carbonyl)piperidin-4- yl)butyl)-3-(pyridin-3- yl)acrylamide | (E)-N-(4-(1-(6-(4-(4- (2-(2-((2-(2,6- dioxopiperidin-3-yl)- 1,3-dioxoisoindolin-4- yl)thio)ethoxy)acetyl) piperazin-1- yl)piperidin- 1-yl)pyridazine-3- carbonyl)piperidin-4- yl)butyl)-3-(pyridin-3- yl)acrylamide |
| | 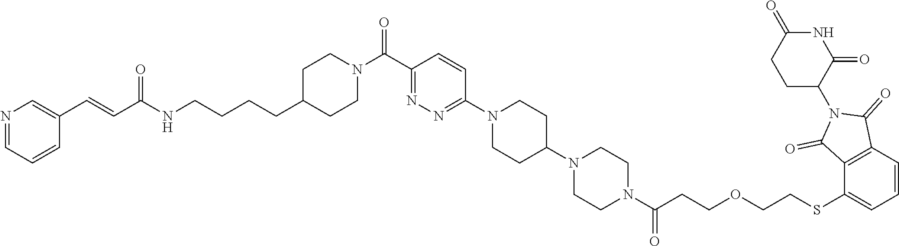 [SMILES: O=C(/C=C/c1cccnc1)NCCCCC1CCN(C(=O)c2ccc(N3CCC(N4CCN(C(=O)CCOCCSc5cccc6c5C(=O)N(C5CCC(=O)NC5=O)C6=O)CC4)CC3)nn2)CC1] | (E)-N-(4-(1-(6-(4-(4-(3- (2-((2-(2,6- dioxopiperidin-3-yl)- 1,3-dioxoisoindolin-4- yl)thio)ethoxy) propanoyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (3-(2-((2-(2,6- dioxopiperidin-3-yl)- 1,3-dioxoisoindolin- 4-yl)thio)ethoxy) propanoyl) piperazin-1- yl)piperidin-1- yl)pyridazine-3- carbonyl)piperidin-4- yl)butyl)-3-(pyridin-3- yl)acrylamide |
| | 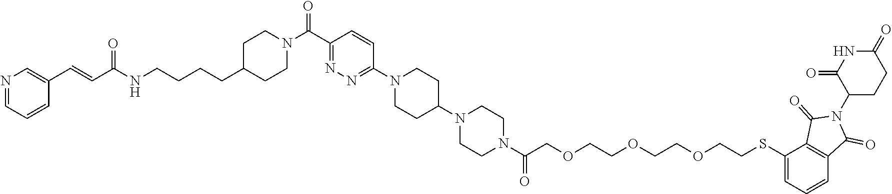 [SMILES: O=C(/C=C/c1cccnc1)NCCCCC1CCN(C(=O)c2ccc(N3CCC(N4CCN(C(=O)COCCOCCOCCSc5cccc6c5C(=O)N(C5CCC(=O)NC5=O)C6=O)CC4)CC3)nn2)CC1] | (E)-N-(4-(1-(6-(4-(4-(2- (2-(2-(2-((2-(2,6- dioxopiperidin-3-yl)- 1,3-dioxoisoindolin-4- yl)thio)ethoxy)ethoxy) ethoxy)acetyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (2-(2-(2-(2-((2-(2,6- dioxopiperidin-3-yl)- 1,3-dioxoisoindolin-4- yl)thio)ethoxy)ethoxy) ethoxy)acetyl) piperazin- 1-yl)piperidin-1- yl)pyridazine-3- carbonyl)piperidin-4- yl)butyl)-3-(pyridin-3- yl)acrylamide |
| | 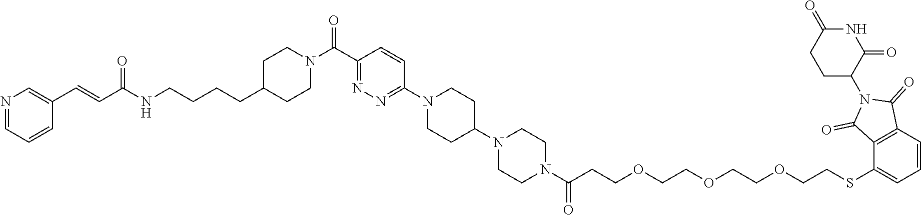 [SMILES: O=C(/C=C/c1cccnc1)NCCCCC1CCN(C(=O)c2ccc(N3CCC(N4CCN(C(=O)CCOCCOCCOCCSc5cccc6c5C(=O)N(C5CCC(=O)NC5=O)C6=O)CC4)CC3)nn2)CC1] | (E)-N-(4-(1-(6-(4-(4-(3- (2-(2-(2-((2-(2,6- dioxopiperidin-3-yl)- 1,3-dioxoisoindolin-4- yl)thio)ethoxy)ethoxy) ethoxy)propanoyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (3-(2-(2-(2-((2-(2,6- dioxopiperidin-3-yl)- 1,3-dioxoisoindolin-4- yl)thio)ethoxy)ethoxy) ethoxy)propanoyl) piperazin-1- yl)piperidin-1- yl)pyridazine-3- carbonyl)piperidin-4- yl)butyl)-3-(pyridin-3- yl)acrylamide |
| | 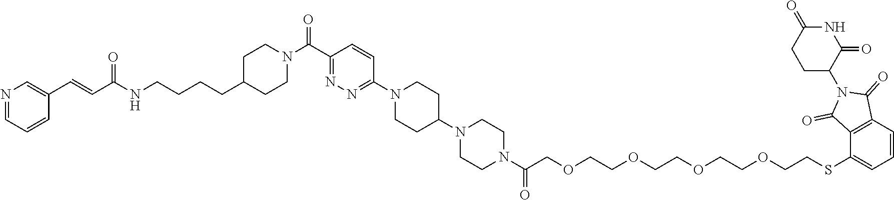 [SMILES: O=C(/C=C/c1cccnc1)NCCCCC1CCN(C(=O)c2ccc(N3CCC(N4CCN(C(=O)COCCOCCOCCOCCSc5cccc6c5C(=O)N(C5CCC(=O)NC5=O)C6=O)CC4)CC3)nn2)CC1] | (E)-N-(4-(1-(6-(4-(4- (14-((2-(2,6- dioxopiperidin-3-yl)- 1,3-dioxoisoindolin-4- yl)thio)-3,6,9,12- tetraoxatetradecanoyl) piperazin-1-yl)piperidin- 1-yl)pyridazine-3- carbonyl)piperidin-4- yl)butyl)-3-(pyridin-3- yl)acrylamide | (E)-N-(4-(1-(6-(4-(4- (14-((2-(2,6- dioxopiperidin-3-yl)- 1,3-dioxoisoindolin-4- yl)thio)-3,6,9,12- tetraoxatetradecanoyl) piperazin-1- yl)piperidin- 1-yl)pyridazine-3- carbonyl)piperidin-4- yl)butyl)-3-(pyridin-3- yl)acrylamide |
| | 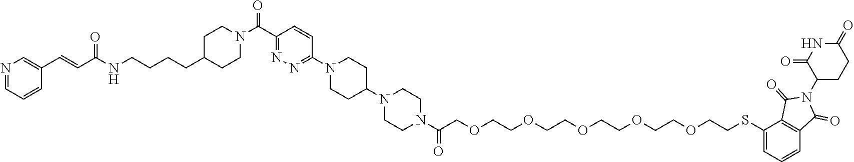 [SMILES: O=C(/C=C/c1cccnc1)NCCCCC1CCN(C(=O)c2ccc(N3CCC(N4CCN(C(=O)COCCOCCOCCOCCOCCSc5cccc6c5C(=O)N(C5CCC(=O)NC5=O)C6=O)CC4)CC3)nn2)CC1] | (E)-N-(4-(1-(6-(4-(4- (17-((2-(2,6- dioxopiperidin-3-yl)- 1,3-dioxoisoindolin-4- yl)thio)-3,6,9,12,15- pentaoxaheptadecanoyl) piperazin-1-yl)piperidin- 1-yl)pyridazine-3- carbonyl)piperidin-4- yl)butyl)-3-(pyridin-3- | (E)-N-(4-(1-(6-(4-(4- (17-((2-(2,6- dioxopiperidin-3-yl)- 1,3-dioxoisoindolin-4- yl)thio)-3,6,9,12,15- pentaoxaheptadecanoyl) piperazin-1- yl)piperidin-1- yl)pyridazine-3- carbonyl)piperidin-4- |
| | | yl)acrylamide | yl)butyl)-3-(pyridin-3- |
| | | | yl)acrylamide |
| | 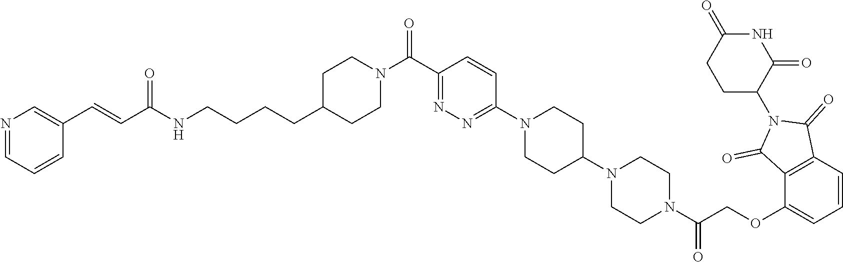 [SMILES: O=C(/C=C/c1cccnc1)NCCCCC1CCN(C(=O)c2ccc(N3CCC(N4CCN(C(=O)COc5cccc6c5C(=O)N(C5CCC(=O)NC5=O)C6=O)CC4)CC3)nn2)CC1] | (E)-N-(4-(1-(6-(4-(4-(2- ((2-(2,6-dioxopiperidin- 3-yl)-1,3- dioxoisoindolin-4- yl)oxy)acetyl)piperazin- 1-yl)piperidin-1- yl)pyridazine-3- carbonyl)piperidin-4- yl)butyl)-3-(pyridin-3- yl)acrylamide | (E)-N-(4-(1-(6-(4-(4- (2-((2-(2,6- dioxopiperidin-3-yl)- 1,3-dioxoisoindolin-4- yl)oxy)acetyl) piperazin-1- yl)piperidin-1- yl)pyridazine-3- carbonyl)piperidin-4- yl)butyl)-3-(pyridin-3- yl)acrylamide |
| | 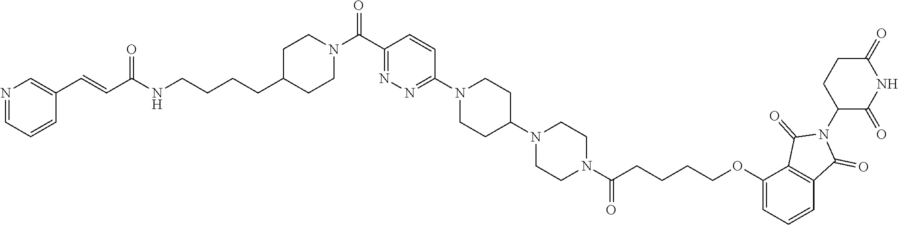 [SMILES: O=C(/C=C/c1cccnc1)NCCCCC1CCN(C(=O)c2ccc(N3CCC(N4CCN(C(=O)CCCCOc5cccc6c5C(=O)N(C5CCC(=O)NC5=O)C6=O)CC4)CC3)nn2)CC1] | (E)-N-(4-(1-(6-(4-(4-(5- ((2-(2,6-dioxopiperidin- 3-yl)-1,3- dioxoisoindolin-4- yl)oxy)pentanoyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (5-((2-(2,6- dioxopiperidin-3-yl)- 1,3-dioxoisoindolin-4- yl)oxy)pentanoyl) piperazin-1-yl) piperidin-1- yl)pyridazine-3- carbonyl)piperidin-4- yl)butyl)-3-(pyridin-3- yl)acrylamide |
| | 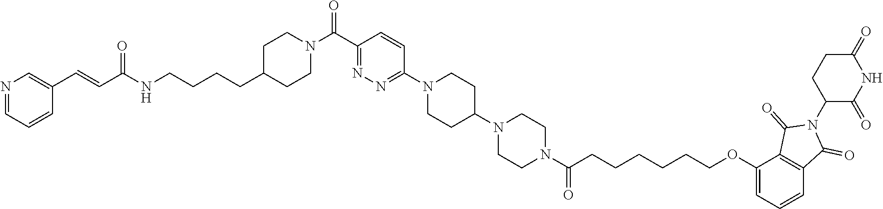 [SMILES: O=C(/C=C/c1cccnc1)NCCCCC1CCN(C(=O)c2ccc(N3CCC(N4CCN(C(=O)CCCCCCOc5cccc6c5C(=O)N(C5CCC(=O)NC5=O)C6=O)CC4)CC3)nn2)CC1] | (E)-N-(4-(1-(6-(4-(4-(7- ((2-(2,6-dioxopiperidin- 3-yl)-1,3- dioxoisoindolin-4- yl)oxy)heptanoyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (7-((2-(2,6- dioxopiperidin-3-yl)- 1,3-dioxoisoindolin-4- yl)oxy)heptanoyl) piperazin-1- yl)piperidin-1- yl)pyridazine-3- carbonyl)piperidin-4- yl)butyl)-3-(pyridin-3- yl)acrylamide |
| | 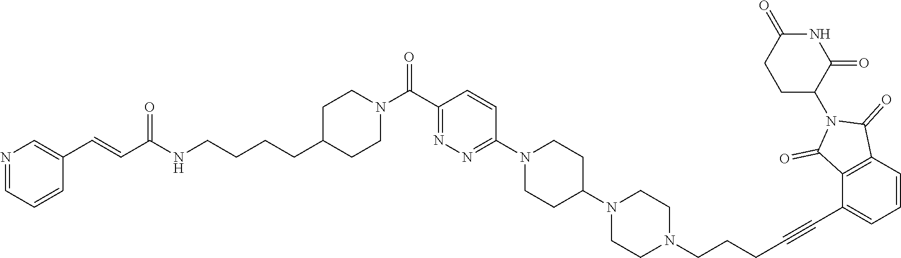 [SMILES: O=C(/C=C/c1cccnc1)NCCCCC1CCN(C(=O)c2ccc(N3CCC(N4CCN(CCCC#Cc5cccc6c5C(=O)N(C5CCC(=O)NC5=O)C6=O)CC4)CC3)nn2)CC1] | (E)-N-(4-(1-(6-(4-(4-(5- (2-(2,6-dioxopiperidin- 3-yl)-1,3- dioxoisoindolin-4- yl)pent-4-yn-1- yl)piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (5-(2-(2,6- dioxopiperidin-3-yl)- 1,3-dioxoisoindolin-4- yl)pent-4-yn-1- yl)piperazin-1- yl)piperidin-1- yl)pyridazine-3- carbonyl)piperidin-4- yl)butyl)-3-(pyridin-3- yl)acrylamide |
| | 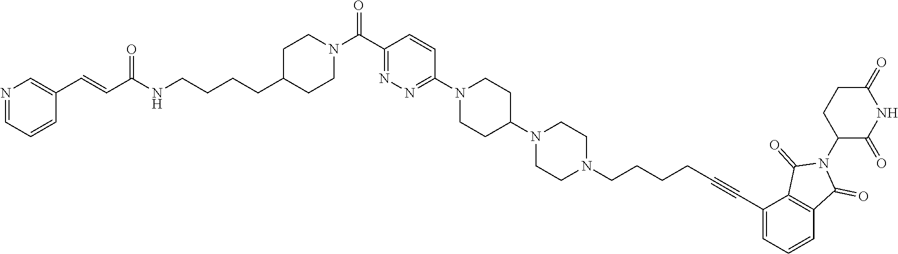 [SMILES: O=C(/C=C/c1cccnc1)NCCCCC1CCN(C(=O)c2ccc(N3CCC(N4CCN(CCCCC#Cc5cccc6c5C(=O)N(C5CCC(=O)NC5=O)C6=O)CC4)CC3)nn2)CC1] | (E)-N-(4-(1-(6-(4-(4-(6- (2-(2,6-dioxopiperidin- 3-yl)-1,3- dioxoisoindolin-4- yl)hex-5-yn-1- yl)piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (6-(2-(2,6- dioxopiperidin-3-yl)- 1,3-dioxoisoindolin-4- yl)hex-5-yn-1- yl)piperazin-1- yl)piperidin-1- yl)pyridazine-3- carbonyl)piperidin-4- yl)butyl)-3-(pyridin-3- yl)acrylamide |
| | 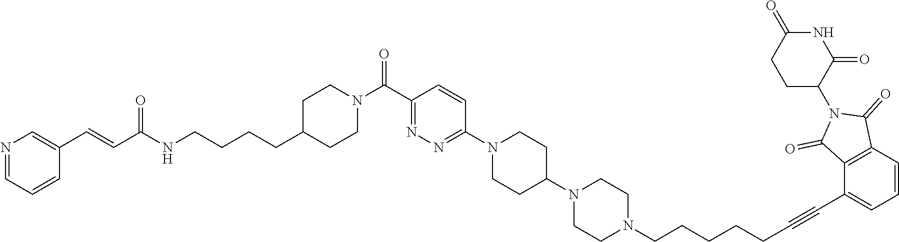 [SMILES: O=C(/C=C/c1cccnc1)NCCCCC1CCN(C(=O)c2ccc(N3CCC(N4CCN(CCCCCC#Cc5cccc6c5C(=O)N(C5CCC(=O)NC5=O)C6=O)CC4)CC3)nn2)CC1] | (E)-N-(4-(1-(6-(4-(4-(7- (2-(2,6-dioxopiperidin- 3-yl)-1,3- dioxoisoindolin-4- yl)hept-6-yn-1- yl)piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (7-(2-(2,6- dioxopiperidin-3-yl)- 1,3-dioxoisoindolin-4- yl)hept-6-yn-1- yl)piperazin-1- yl)piperidin-1- yl)pyridazine-3- carbonyl)piperidin-4- yl)butyl)-3-(pyridin-3- yl)acrylamide |
| | 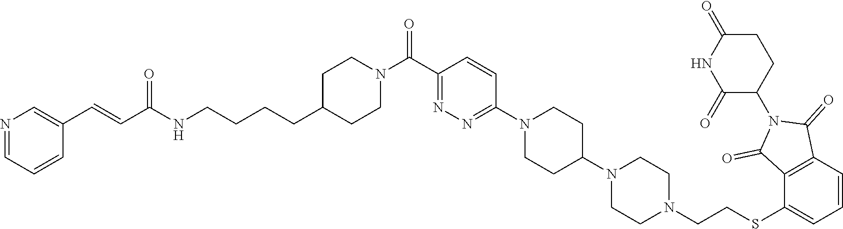 [SMILES: O=C(/C=C/c1cccnc1)NCCCCC1CCN(C(=O)c2ccc(N3CCC(N4CCN(CCSc5cccc6c5C(=O)N(C5CCC(=O)NC5=O)C6=O)CC4)CC3)nn2)CC1] | (E)-N-(4-(1-(6-(4-(4-(2- ((2-(2,6-dioxopiperidin- 3-yl)-1,3- dioxoisoindolin-4- yl)thio)ethyl)piperazin- 1-yl)piperidin-1- yl)pyridazine-3- carbonyl)piperidin-4- yl)butyl)-3-(pyridin-3- yl)acrylamide | (E)-N-(4-(1-(6-(4-(4- (2-((2-(2,6- dioxopiperidin-3-yl)- 1,3-dioxoisoindolin-4- yl)thio)ethyl)piperazin- 1-yl)piperidin-1 - yl)pyridazine-3- carbonyl)piperidin-4- yl)butyl)-3-(pyridin-3- yl)acrylamide |
| | 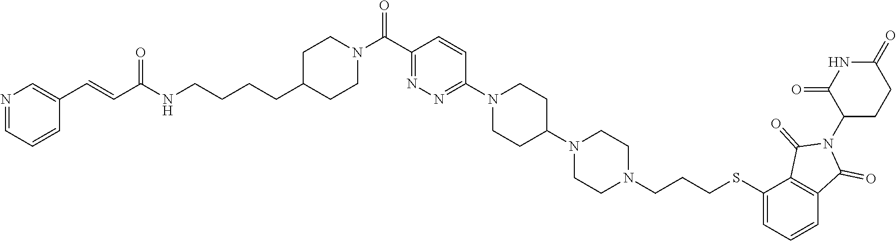 [SMILES: O=C(/C=C/c1cccnc1)NCCCCC1CCN(C(=O)c2ccc(N3CCC(N4CCN(CCCSc5cccc6c5C(=O)N(C5CCC(=O)NC5=O)C6=O)CC4)CC3)nn2)CC1] | (E)-N-(4-(1-(6-(4-(4-(3- ((2-(2,6-dioxopiperidin- 3-yl)-1,3- dioxoisoindolin-4- yl)thio)propyl) piperazin- 1-yl)piperidin-1- yl)pyridazine-3- carbonyl)piperidin-4- yl)butyl)-3-(pyridin-3- yl)acrylamide | (E)-N-(4-(1-(6-(4-(4- (3-((2-(2,6- dioxopiperidin-3-yl)- 1,3-dioxoisoindolin-4- yl)thio)propyl) piperazin-1- yl)piperidin-1- yl)pyridazine-3- carbonyl)piperidin-4- yl)butyl)-3-(pyridin-3- yl)acrylamide |
| | 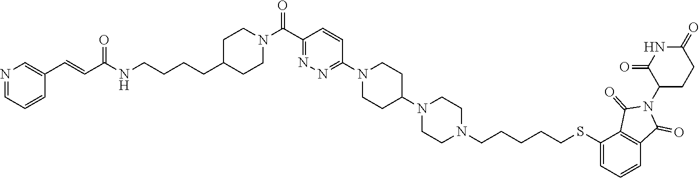 [SMILES: O=C(/C=C/c1cccnc1)NCCCCC1CCN(C(=O)c2ccc(N3CCC(N4CCN(CCCCCSc5cccc6c5C(=O)N(C5CCC(=O)NC5=O)C6=O)CC4)CC3)nn2)CC1] | (E)-N-(4-(1-(6-(4-(4-(5- ((2-(2,6-dioxopiperidin- 3-yl)-1,3- dioxoisoindolin-4- yl)thio)pentyl) piperazin- 1-yl)piperidin-1- yl)pyridazine-3- carbonyl)piperidin-4- yl)butyl)-3-(pyridin-3- yl)acrylamide | (E)-N-(4-(1-(6-(4-(4- (5-((2-(2,6- dioxopiperidin-3-yl)- 1,3-dioxoisoindolin-4- yl)thio)pentyl) piperazin-1- yl)piperidin-1- yl)pyridazine-3- carbonyl)piperidin-4- yl)butyl)-3-(pyridin-3- yl)acrylamide |
| | 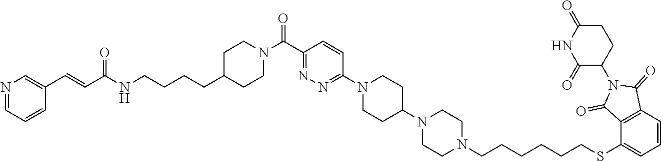 [SMILES: O=C(/C=C/c1cccnc1)NCCCCC1CCN(C(=O)c2ccc(N3CCC(N4CCN(CCCCCCSc5cccc6c5C(=O)N(C5CCC(=O)NC5=O)C6=O)CC4)CC3)nn2)CC1] | (E)-N-(4-(1-(6-(4-(4-(6- ((2-(2,6-dioxopiperidin- 3-yl)-1,3- dioxoisoindolin-4- yl)thio)hexyl)piperazin- 1-yl)piperidin-1- yl)pyridazine-3- carbonyl)piperidin-4- yl)butyl)-3-(pyridin-3- yl)acrylamide | (E)-N-(4-(1-(6-(4-(4- (6-((2-(2,6- dioxopiperidin-3-yl)- 1,3-dioxoisoindolin-4- yl)thio)hexyl) piperazin-1- yl)piperidin-1- yl)pyridazine-3- carbonyl)piperidin-4- yl)butyl)-3-(pyridin-3- yl)acrylamide |
| | 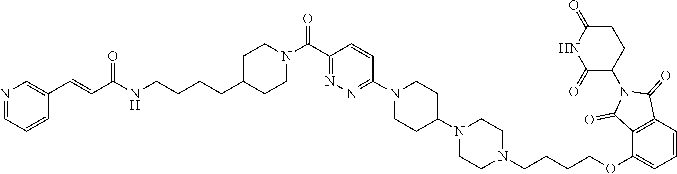 [SMILES: O=C(/C=C/c1cccnc1)NCCCCC1CCN(C(=O)c2ccc(N3CCC(N4CCN(CCCCOc5cccc6c5C(=O)N(C5CCC(=O)NC5=O)C6=O)CC4)CC3)nn2)CC1] | (E)-N-(4-(1-(6-(4-(4-(4- ((2-(2,6-dioxopiperidin- 3-yl)-1,3- dioxoisoindolin-4- yl)oxy)butyl)piperazin- 1-yl)piperidin-1- yl)pyridazine-3- carbonyl)piperidin-4- yl)butyl)-3-(pyridin-3- yl)acrylamide | (E)-N-(4-(1-(6-(4-(4- (4-((2-(2,6- dioxopiperidin-3-yl)- 1,3-dioxoisoindolin-4- yl)oxy)butyl) piperazin-1-yl) piperidin-1- yl)pyridazine-3- carbonyl)piperidin-4- yl)butyl)-3-(pyridin-3- yl)acrylamide |
| | 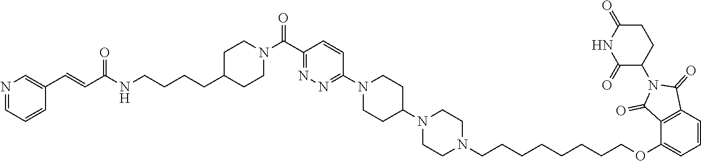 [SMILES: O=C(/C=C/c1cccnc1)NCCCCC1CCN(C(=O)c2ccc(N3CCC(N4CCN(CCCCCCCCOc5cccc6c5C(=O)N(C5CCC(=O)NC5=O)C6=O)CC4)CC3)nn2)CC1] | (E)-N-(4-(1-(6-(4-(4-(8- ((2-(2,6-dioxopiperidin- 3-yl)-1,3- dioxoisoindolin-4- yl)oxy)octyl)piperazin- 1-yl)piperidin-1- yl)pyridazine-3- carbonyl)piperidin-4- yl)butyl)-3-(pyridin-3- yl)acrylamide | (E)-N-(4-(1-(6-(4-(4- (8-((2-(2,6- dioxopiperidin-3-yl)- 1,3-dioxoisoindolin-4- yl)oxy)octyl)piperazin- 1-yl)piperidin-1- yl)pyridazine-3- carbonyl)piperidin-4- yl)butyl)-3-(pyridin-3- yl)acrylamide |
| | 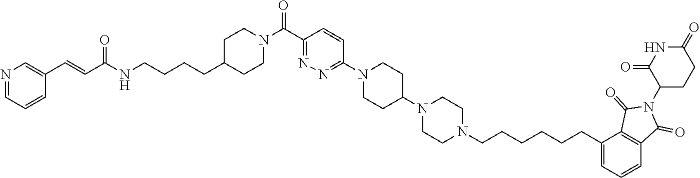 [SMILES: O=C(/C=C/c1cccnc1)NCCCCC1CCN(C(=O)c2ccc(N3CCC(N4CCN(CCCCCCc5cccc6c5C(=O)N(C5CCC(=O)NC5=O)C6=O)CC4)CC3)nn2)CC1] | (E)-N-(4-(1-(6-(4-(4-(6- (2-(2,6-dioxopiperidin- 3-yl)-1,3- dioxoisoindolin-4- yl)hexyl)piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (6-(2-(2,6- dioxopiperidin-3-yl)- 1,3-dioxoisoindolin-4- yl)hexyl)piperazin-1- yl)piperidin-1- yl)pyridazine-3- carbonyl)piperidin-4- yl)butyl)-3-(pyridin-3- yl)acrylamide |
| | 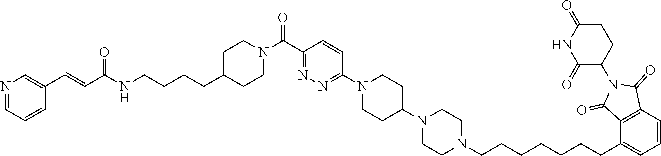 [SMILES: O=C(/C=C/c1cccnc1)NCCCCC1CCN(C(=O)c2ccc(N3CCC(N4CCN(CCCCCCCc5cccc6c5C(=O)N(C5CCC(=O)NC5=O)C6=O)CC4)CC3)nn2)CC1] | (E)-N-(4-(1-(6-(4-(4-(7- (2-(2,6-dioxopiperidin- 3-yl)-1,3- dioxoisoindolin-4- yl)heptyl)piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (7-(2-(2,6- dioxopiperidin-3-yl)- 1,3-dioxoisoindolin-4- yl)heptyl)piperazin-1- yl)piperidin-1- yl)pyridazine-3- carbonyl)piperidin-4- yl)butyl)-3-(pyridin-3- yl)acrylamide |
| | 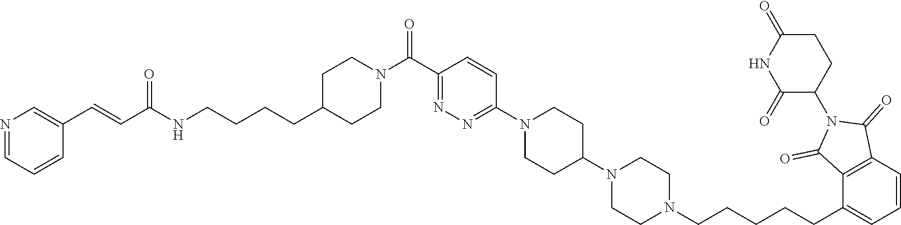 [SMILES: O=C(/C=C/c1cccnc1)NCCCCC1CCN(C(=O)c2ccc(N3CCC(N4CCN(CCCCCc5cccc6c5C(=O)N(C5CCC(=O)NC5=O)C6=O)CC4)CC3)nn2)CC1] | (E)-N-(4-(1-(6-(4-(4-(5- (2-(2,6-dioxopiperidin- 3-yl)-1,3- dioxoisoindolin-4- yl)pentyl)piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (5-(2-(2,6- dioxopiperidin-3-yl)- 1,3-dioxoisoindolin-4- yl)pentyl)piperazin-1- yl)piperidin-1- yl)pyridazine-3- carbonyl)piperidin-4- yl)butyl)-3-(pyridin-3- yl)acrylamide |
| | 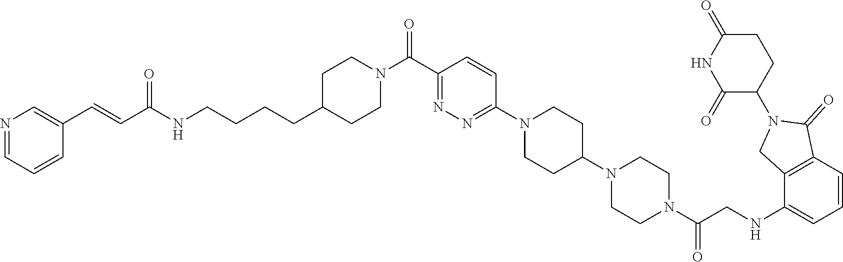 [SMILES: O=C(/C=C/c1cccnc1)NCCCCC1CCN(C(=O)c2ccc(N3CCC(N4CCN(C(=O)CNc5cccc6c5CN(C5CCC(=O)NC5=O)C6=O)CC4)CC3)nn2)CC1] | (E)-N-(4-(1-(6-(4-(4- ((2-(2,6-dioxopiperidin- 3-yl)-1-oxoisoindolin-4- yl)glycyl)piperazin-1- yl)piperidin-1- yl)pyridazine-3- carbonyl)piperidin-4- yl)butyl)-3-(pyridin-3- yl)acrylamide | (E)-N-(4-(1-(6-(4-(4- ((2-(2,6- dioxopiperidin-3-yl)- 1-oxoisoindolin-4- yl)glycyl)piperazin- 1-yl)piperidin-1- yl)pyridazine-3- carbonyl)piperidin-4- yl)butyl)-3-(pyridin-3- yl)acrylamide |
| | 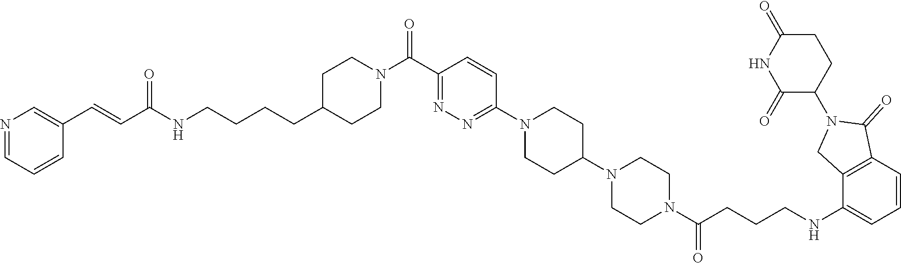 [SMILES: O=C(/C=C/c1cccnc1)NCCCCC1CCN(C(=O)c2ccc(N3CCC(N4CCN(C(=O)CCCNc5cccc6c5CN(C5CCC(=O)NC5=O)C6=O)CC4)CC3)nn2)CC1] | (E)-N-(4-(1-(6-(4-(4-(4- ((2-(2,6-dioxopiperidin- 3-yl)-1-oxoisoindolin-4- yl)amino)butanoyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (4-((2-(2,6- dioxopiperidin-3-yl)- 1-oxoisoindolin-4- yl)amino)butanoyl)p iperazin-1- yl)piperidin-1- yl)pyridazine-3- carbonyl)piperidin-4- yl)butyl)-3-(pyridin-3- yl)acrylamide |
| | 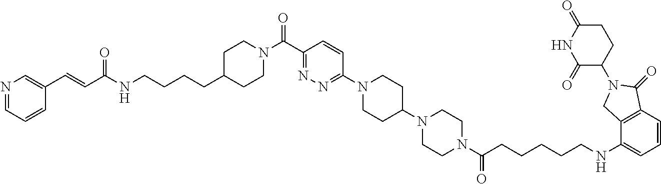 [SMILES: O=C(/C=C/c1cccnc1)NCCCCC1CCN(C(=O)c2ccc(N3CCC(N4CCN(C(=O)CCCCCNc5cccc6c5CN(C5CCC(=O)NC5=O)C6=O)CC4)CC3)nn2)CC1] | (E)-N-(4-(1-(6-(4-(4-(6- ((2-(2,6-dioxopiperidin- 3-yl)-1-oxoisoindolin- 4-yl)amino)hexanoyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (6-((2-(2,6- dioxopiperidin-3-yl)- 1-oxoisoindolin-4- yl)amino)hexanoyl) piperazin-1- yl)piperidin-1- yl)pyridazine-3- carbonyl)piperidin-4- yl)butyl)-3-(pyridin-3- yl)acrylamide |
| | 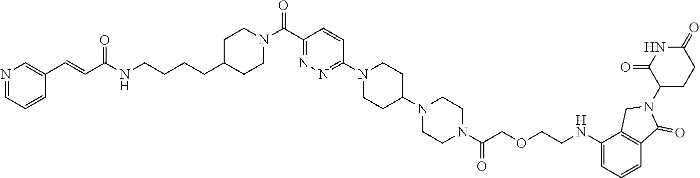 [SMILES: O=C(/C=C/c1cccnc1)NCCCCC1CCN(C(=O)c2ccc(N3CCC(N4CCN(C(=O)COCCNc5cccc6c5CN(C5CCC(=O)NC5=O)C6=O)CC4)CC3)nn2)CC1] | (E)-N-(4-(1-(6-(4-(4-(2- (2-((2-(2,6- dioxopiperidin-3-yl)-1- oxoisoindolin-4- yl)amino)ethoxy)acetyl) piperazin-1-yl)piperidin- 1-yl)pyridazine-3- carbonyl)piperidin-4- yl)butyl)-3-(pyridin-3- yl)acrylamide | (E)-N-(4-(1-(6-(4-(4- (2-(2-((2-(2,6- dioxopiperidin-3-yl)- 1-oxoisoindolin-4- yl)amino)ethoxy) acetyl)piperazin-1- yl)piperidin-1- yl)pyridazine-3- carbonyl)piperidin-4- yl)butyl)-3-(pyridin-3- yl)acrylamide |
| | 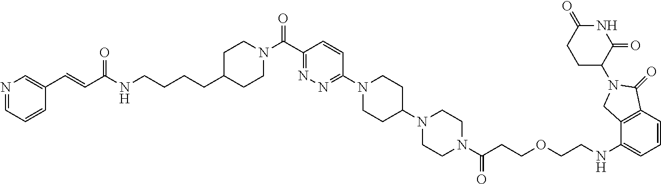 [SMILES: O=C(/C=C/c1cccnc1)NCCCCC1CCN(C(=O)c2ccc(N3CCC(N4CCN(C(=O)CCOCCNc5cccc6c5CN(C5CCC(=O)NC5=O)C6=O)CC4)CC3)nn2)CC1] | (E)-N-(4-(1-(6-(4-(4-(3- (2-((2-(2,6- dioxopiperidin-3-yl)-1- oxoisoindolin-4- yl)amino)ethoxy) propanoyl)piperazin- 1-yl)piperidin-1- yl)pyridazine-3- carbonyl)piperidin-4- yl)butyl)-3-(pyridin-3- yl)acrylamide | (E)-N-(4-(1-(6-(4-(4- (3-(2-((2-(2,6- dioxopiperidin-3-yl)- 1-oxoisoindolin-4- yl)amino)ethoxy) propanoyl)piperazin- 1-yl)piperidin-1- yl)pyridazine-3- carbonyl)piperidin-4- yl)butyl)-3-(pyridin-3- yl)acrylamide |
| | 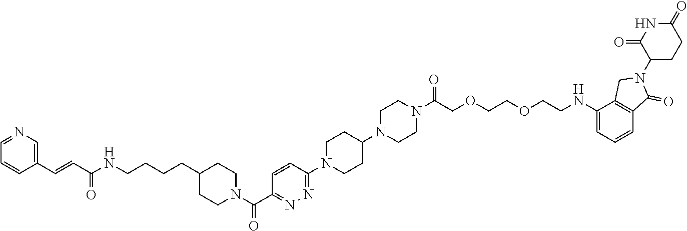 [SMILES: O=C(/C=C/c1cccnc1)NCCCCC1CCN(C(=O)c2ccc(N3CCC(N4CCN(C(=O)COCCOCCNc5cccc6c5CN(C5CCC(=O)NC5=O)C6=O)CC4)CC3)nn2)CC1] | (E)-N-(4-(1-(6-(4-(4-(2- (2-(2-((2-(2,6- dioxopiperidin-3-yl)-1- oxoisoindolin-4- yl)amino)ethoxy) ethoxy)acetyl) piperazin-1 - yl)piperidin-1- yl)pyridazine-3- carbonyl)piperidin-4- yl)butyl)-3-(pyridin-3- yl)acrylamide | (E)-N-(4-(1-(6-(4-(4- (2-(2-(2-((2-(2,6- dioxopiperidin-3-yl)- 1-oxoisoindolin-4- yl)amino)ethoxy) ethoxy)acetyl) piperazin-1- yl)piperidin-1- yl)pyridazine-3- carbonyl)piperidin-4- yl)butyl)-3-(pyridin-3- yl)acrylamide |
| | 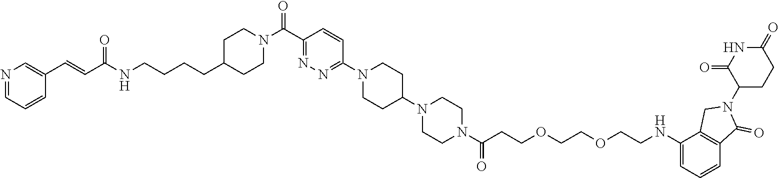 [SMILES: O=C(/C=C/c1cccnc1)NCCCCC1CCN(C(=O)c2ccc(N3CCC(N4CCN(C(=O)CCOCCOCCNc5cccc6c5CN(C5CCC(=O)NC5=O)C6=O)CC4)CC3)nn2)CC1] | (E)-N-(4-(1-(6-(4-(4-(3- (2-(2-((2-(2,6- dioxopiperidin-3-yl)-1- oxoisoindolin-4- yl)amino)ethoxy) ethoxy)propanoyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (3-(2-(2-((2-(2,6- dioxopiperidin-3-yl)- 1-oxoisoindolin-4- yl)amino)ethoxy) ethoxy)propanoyl) piperazin- 1-yl)piperidin-1- yl)pyridazine-3- carbonyl)piperidin-4- yl)butyl)-3-(pyridin-3- yl)acrylamide |
| | 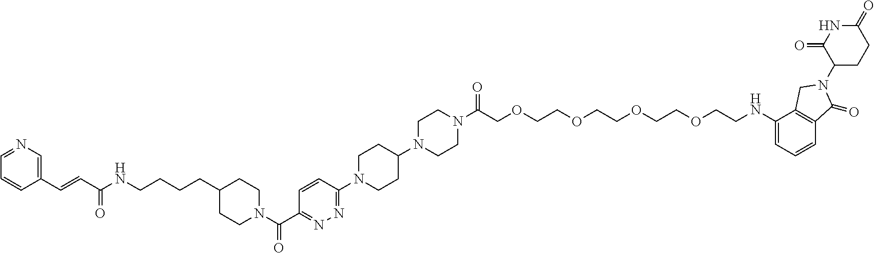 [SMILES: O=C(/C=C/c1cccnc1)NCCCCC1CCN(C(=O)c2ccc(N3CCC(N4CCN(C(=O)COCCOCCOCCOCCNc5cccc6c5CN(C5CCC(=O)NC5=O)C6=O)CC4)CC3)nn2)CC1] | (E)-N-(4-(1-(6-(4-(4- (14-((2-(2,6- dioxopiperidin-3-yl)- 1-oxoisoindolin-4- yl)amino)-3,6,9,12- tetraoxatetradecanoyl) piperazin-1- yl)piperidin- 1-yl)pyridazine-3- carbonyl)piperidin-4- yl)butyl)-3-(pyridin-3- yl)acrylamide | (E)-N-(4-(1-(6-(4-(4- (14-((2-(2,6- dioxopiperidin-3-yl)- 1-oxoisoindolin-4- yl)amino)-3,6,9,12- tetraoxatetradecanoyl) piperazin-1- yl)piperidin- 1-yl)pyridazine-3- carbonyl)piperidin-4- yl)butyl)-3-(pyridin-3- yl)acrylamide |
| | 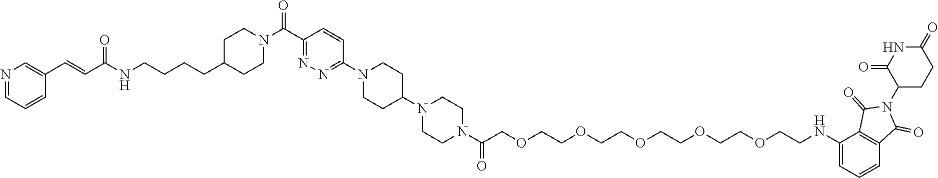 [SMILES: O=C(/C=C/c1cccnc1)NCCCCC1CCN(C(=O)c2ccc(N3CCC(N4CCN(C(=O)COCCOCCOCCOCCOCCNc5cccc6c5C(=O)N(C5CCC(=O)NC5=O)C6=O)CC4)CC3)nn2)CC1] | (E)-N-(4-(1-(6-(4-(4-(1- ((2-(2,6-dioxopiperidin- 3-yl)-1-oxoisoindolin-4- yl)amino)-3,6,9,12- tetraoxapentadecan-15- oyl)piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (1-((2-(2,6- dioxopiperidin-3-yl)- 1-oxoisoindolin-4- yl)amino)-3,6,9,12- tetraoxapentadecan- 15-oyl)piperazin-1- yl)piperidin-1- yl)pyridazine-3- carbonyl)piperidin-4- yl)butyl)-3-(pyridin- 3-yl)acrylamide |
| | 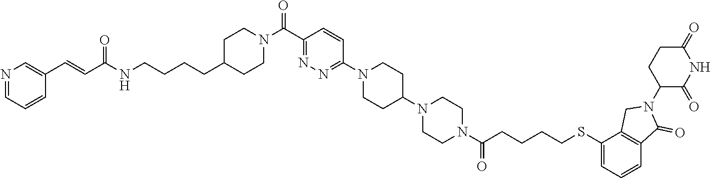 [SMILES: O=C(/C=C/c1cccnc1)NCCCCC1CCN(C(=O)c2ccc(N3CCC(N4CCN(C(=O)CCCCSc5cccc6c5CN(C5CCC(=O)NC5=O)C6=O)CC4)CC3)nn2)CC1] | (E)-N-(4-(1-(6-(4-(4-(5- ((2-(2,6-dioxopiperidin- 3-yl)-1-oxoisoindolin-4- yl)thio)pentanoyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (5-((2-(2,6- dioxopiperidin-3-yl)- 1-oxoisoindolin-4- yl)thio)pentanoyl) piperazin-1- yl)piperidin-1- yl)pyridazine-3- carbonyl)piperidin-4- yl)butyl)-3-(pyridin-3- yl)acrylamide |
| | 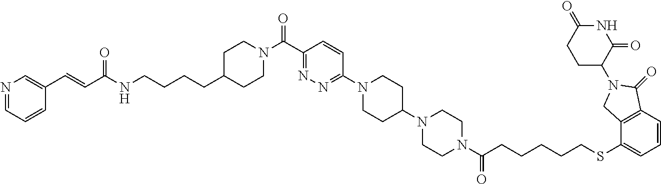 [SMILES: O=C(/C=C/c1cccnc1)NCCCCC1CCN(C(=O)c2ccc(N3CCC(N4CCN(C(=O)CCCCCSc5cccc6c5CN(C5CCC(=O)NC5=O)C6=O)CC4)CC3)nn2)CC1] | (E)-N-(4-(1-(6-(4-(4-(6- ((2-(2,6-dioxopiperidin- 3-yl)-1-oxoisoindolin- 4-yl)thio)hexanoyl) piperazin-1-yl) piperidin-1- yl)pyridazine-3- carbonyl)piperidin-4- yl)butyl)-3-(pyridin-3- yl)acrylamide | (E)-N-(4-(1-(6-(4-(4- (6-((2-(2,6- dioxopiperidin-3-yl)- 1-oxoisoindolin-4- yl)thio)hexanoyl) piperazin-1- yl)piperidin-1- yl)pyridazine-3- carbonyl)piperidin-4- yl)butyl)-3-(pyridin- 3-yl)acrylamide |
| | 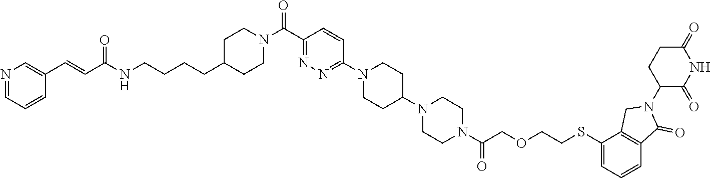 [SMILES: O=C(/C=C/c1cccnc1)NCCCCC1CCN(C(=O)c2ccc(N3CCC(N4CCN(C(=O)COCCSc5cccc6c5CN(C5CCC(=O)NC5=O)C6=O)CC4)CC3)nn2)CC1] | (E)-N-(4-(1-(6-(4-(4-(2- (2-((2-(2,6- dioxopiperidin-3-yl)- 1-oxoisoindolin-4- yl)thio)ethoxy)acetyl) piperazin-1- yl)piperidin- 1-yl)pyridazine-3- carbonyl)piperidin-4- yl)butyl)-3-(pyridin-3- yl)acrylamide | (E)-N-(4-(1-(6-(4-(4- (2-(2-((2-(2,6- dioxopiperidin-3-yl)- 1-oxoisoindolin-4- yl)thio)ethoxy)acetyl) piperazin-1- yl)piperidin- 1-yl)pyridazine-3- carbonyl)piperidin-4- yl)butyl)-3-(pyridin-3- yl)acrylamide |
| | 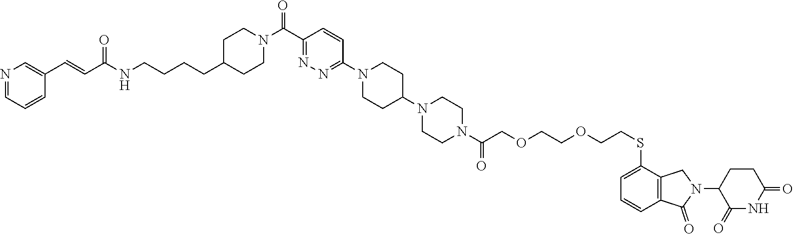 [SMILES: O=C(/C=C/c1cccnc1)NCCCCC1CCN(C(=O)c2ccc(N3CCC(N4CCN(C(=O)COCCOCCSc5cccc6c5CN(C5CCC(=O)NC5=O)C6=O)CC4)CC3)nn2)CC1] | (E)-N-(4-(1-(6-(4-(4-(2- (2-(2-((2-(2,6- dioxopiperidin-3-yl)- 1-oxoisoindolin-4- yl)thio)ethoxy)ethoxy) acetyl)piperazin-1- yl)piperidin-1- yl)pyridazine-3- carbonyl)piperidin-4- yl)butyl)-3-(pyridin- 3-yl)acrylamide | (E)-N-(4-(1-(6-(4-(4- (2-(2-(2-((2-(2,6- dioxopiperidin-3-yl)- 1-oxoisoindolin-4- yl)thio)ethoxy) ethoxy)acetyl) piperazin-1- yl)piperidin-1- yl)pyridazine-3- carbonyl)piperidin-4- yl)butyl)-3-(pyridin- 3-yl)acrylamide |
| | 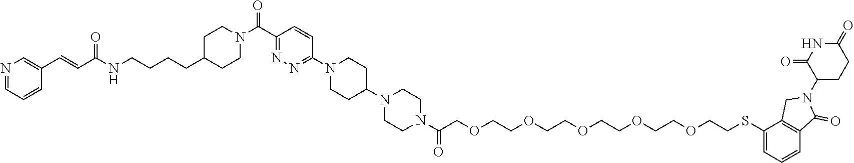 [SMILES: O=C(/C=C/c1cccnc1)NCCCCC1CCN(C(=O)c2ccc(N3CCC(N4CCN(C(=O)COCCOCCOCCOCCOCCSc5cccc6c5CN(C5CCC(=O)NC5=O)C6=O)CC4)CC3)nn2)CC1] | (E)-N-(4-(1-(6-(4-(4- (17-((2-(2,6- dioxopiperidin-3-yl)- 1-oxoisoindolin-4- yl)thio)-3,6,9,12,15- pentaoxaheptadecanoyl) piperazin-1-yl) piperidin- 1-yl)pyridazine-3- carbonyl)piperidin-4- yl)butyl)-3-(pyridin-3- yl)acrylamide | (E)-N-(4-(1-(6-(4-(4- (17-((2-(2,6- dioxopiperidin-3-yl)- 1-oxoisoindolin-4- yl)thio)-3,6,9,12,15- pentaoxaheptadecanoyl) piperazin-1- yl)piperidin-1- yl)pyridazine-3- carbonyl)piperidin-4- yl)butyl)-3-(pyridin-3- yl)acrylamide |
| | 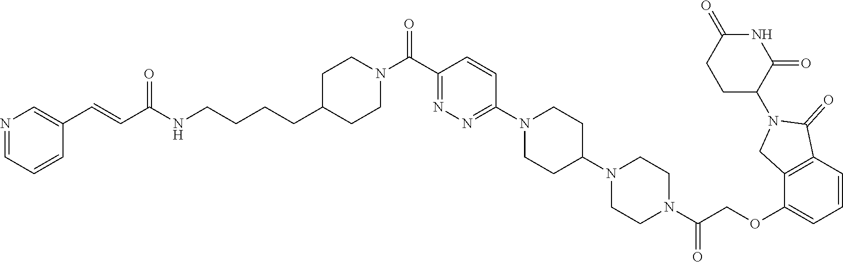 [SMILES: O=C(/C=C/c1cccnc1)NCCCCC1CCN(C(=O)c2ccc(N3CCC(N4CCN(C(=O)COc5cccc6c5CN(C5CCC(=O)NC5=O)C6=O)CC4)CC3)nn2)CC1] | (E)-N-(4-(1-(6-(4-(4-(2- ((2-(2,6-dioxopiperidin- 3-yl)-1-oxoisoindolin-4- yl)oxy)acetyl)piperazin- 1-yl)piperidin-1- yl)pyridazine-3- carbonyl)piperidin-4- yl)butyl)-3-(pyridin-3- yl)acrylamide | (E)-N-(4-(1-(6-(4-(4- (2-((2-(2,6- dioxopiperidin-3-yl)- 1-oxoisoindolin-4- yl)oxy)acetyl) piperazin-1- yl)piperidin-1- yl)pyridazine-3- carbonyl)piperidin-4- yl)butyl)-3-(pyridin- 3-yl)acrylamide |
| | 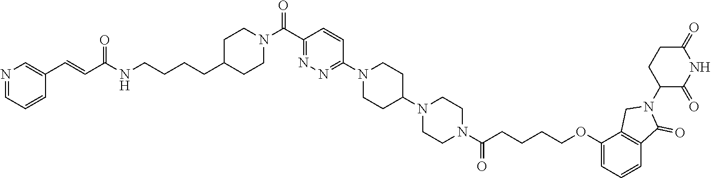 [SMILES: O=C(/C=C/c1cccnc1)NCCCCC1CCN(C(=O)c2ccc(N3CCC(N4CCN(C(=O)CCCCOc5cccc6c5CN(C5CCC(=O)NC5=O)C6=O)CC4)CC3)nn2)CC1] | (E)-N-(4-(1-(6-(4-(4-(5- ((2-(2,6-dioxopiperidin- 3-yl)-1-oxoisoindolin- 4-yl)oxy)pentanoyl) piperazin-1- yl)piperidin-1- yl)pyridazine-3- carbonyl)piperidin-4- yl)butyl)-3-(pyridin- 3-yl)acrylamide | (E)-N-(4-(1-(6-(4-(4- (5-((2-(2,6- dioxopiperidin-3-yl)- 1-oxoisoindolin-4- yl)oxy)pentanoyl) piperazin-1-yl) piperidin-1- yl)pyridazine-3- carbonyl)piperidin-4- yl)butyl)-3-(pyridin- 3-yl)acrylamide |
| | 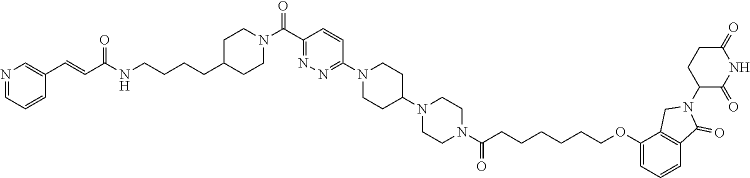 [SMILES: O=C(/C=C/c1cccnc1)NCCCCC1CCN(C(=O)c2ccc(N3CCC(N4CCN(C(=O)CCCCCCOc5cccc6c5CN(C5CCC(=O)NC5=O)C6=O)CC4)CC3)nn2)CC1] | (E)-N-(4-(1-(6-(4-(4-(7- ((2-(2,6-dioxopiperidin- 3-yl)-1-oxoisoindolin- 4-yl)oxy)heptanoyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (7-((2-(2,6- dioxopiperidin-3-yl)- 1-oxoisoindolin-4- yl)oxy)heptanoyl) piperazin-1- yl)piperidin-1- yl)pyridazine-3- carbonyl)piperidin-4- yl)butyl)-3-(pyridin- 3-yl)acrylamide |
| | 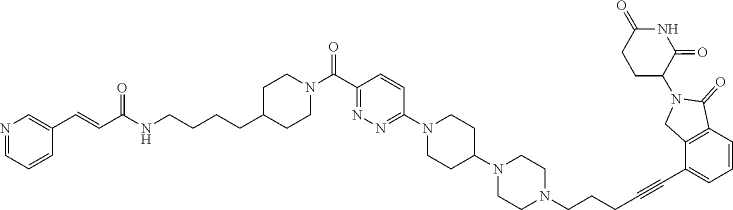 [SMILES: O=C(/C=C/c1cccnc1)NCCCCC1CCN(C(=O)c2ccc(N3CCC(N4CCN(CCCC#Cc5cccc6c5CN(C5CCC(=O)NC5=O)C6=O)CC4)CC3)nn2)CC1] | (E)-N-(4-(1-(6-(4-(4-(5- (2-(2,6-dioxopiperidin- 3-yl)-1-oxoisoindolin- 4-yl)pent-4-yn-1- yl)piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (5-(2-(2,6- dioxopiperidin-3- yl)-1-oxoisoindolin- 4-yl)pent-4-yn-1- yl)piperazin-1- yl)piperidin-1- yl)pyridazine-3- carbonyl)piperidin-4- yl)butyl)-3-(pyridin- 3-yl)acrylamide |
| | 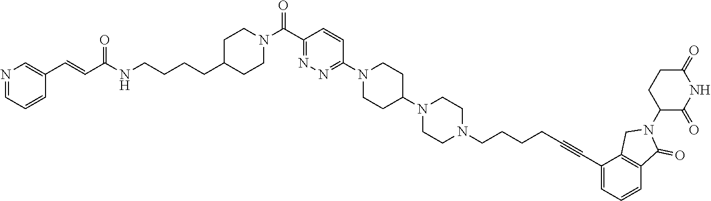 [SMILES: O=C(/C=C/c1cccnc1)NCCCCC1CCN(C(=O)c2ccc(N3CCC(N4CCN(CCCCC#Cc5cccc6c5CN(C5CCC(=O)NC5=O)C6=O)CC4)CC3)nn2)CC1] | (E)-N-(4-(1-(6-(4-(4-(6- (2-(2,6-dioxopiperidin- 3-yl)-1-oxoisoindolin- 4-yl)hex-5-yn-1- yl)piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (6-(2-(2,6- dioxopiperidin-3- yl)-1-oxoisoindolin- 4-yl)hex-5-yn-1- yl)piperazin-1- yl)piperidin-1- yl)pyridazine-3- carbonyl)piperidin-4- yl)butyl)-3-(pyridin- 3-yl)acrylamide |
| | 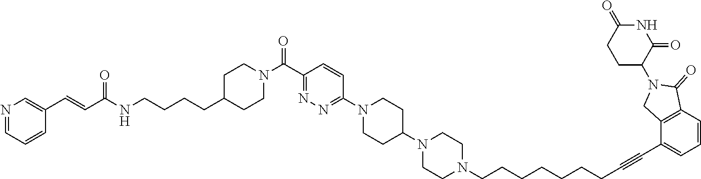 [SMILES: O=C(/C=C/c1cccnc1)NCCCCC1CCN(C(=O)c2ccc(N3CCC(N4CCN(CCCCCCCC#Cc5cccc6c5CN(C5CCC(=O)NC5=O)C6=O)CC4)CC3)nn2)CC1] | (E)-N-(4-(1-(6-(4-(4-(9- (2-(2,6-dioxopiperidin- 3-yl)-1-oxoisoindolin- 4-yl)non-8-yn-1- yl)piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (9-(2-(2,6- dioxopiperidin-3-yl)- 1-oxoisoindolin-4- yl)non-8-yn-1- yl)piperazin-1- yl)piperidin-1- yl)pyridazine-3- carbonyl)piperidin-4- yl)butyl)-3-(pyridin- 3-yl)acrylamide |
| | 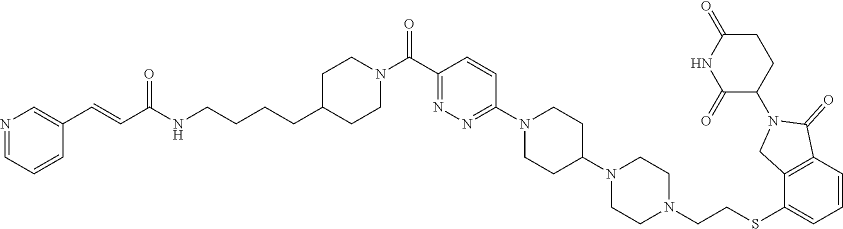 [SMILES: O=C(/C=C/c1cccnc1)NCCCCC1CCN(C(=O)c2ccc(N3CCC(N4CCN(CCSc5cccc6c5CN(C5CCC(=O)NC5=O)C6=O)CC4)CC3)nn2)CC1] | (E)-N-(4-(1-(6-(4-(4-(2- ((2-(2,6-dioxopiperidin- 3-yl)-1-oxoisoindolin- 4-yl)thio)ethyl) piperazin- 1-yl)piperidin-1- yl)pyridazine-3- carbonyl)piperidin-4- yl)butyl)-3-(pyridin-3- yl)acrylamide | (E)-N-(4-(1-(6-(4-(4- (2-((2-(2,6- dioxopiperidin-3-yl)- 1-oxoisoindolin-4- yl)thio)ethyl) piperazin- 1-yl)piperidin-1- yl)pyridazine-3- carbonyl)piperidin-4- yl)butyl)-3-(pyridin- 3-yl)acrylamide |
| | 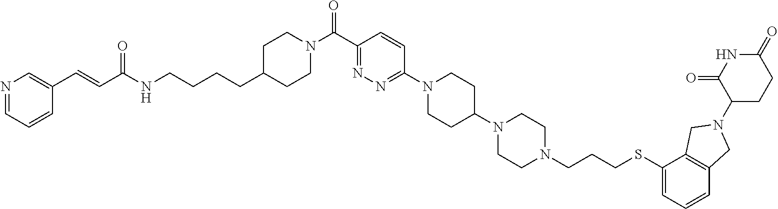 [SMILES: O=C(/C=C/c1cccnc1)NCCCCC1CCN(C(=O)c2ccc(N3CCC(N4CCN(CCCSc5cccc6c5CN(C5CCC(=O)NC5=O)C6)CC4)CC3)nn2)CC1] | (E)-N-(4-(1-(6-(4-(4-(3- ((2-(2,6-dioxopiperidin- 3-yl)-1-oxoisoindolin- 4-yl)thio)propyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (3-((2-(2,6- dioxopiperidin-3-yl)- 1-oxoisoindolin-4- yl)thio)propyl) piperazin-1- yl)piperidin-1- yl)pyridazine-3- carbonyl)piperidin-4- yl)butyl)-3-(pyridin- 3-yl)acrylamide |
| | 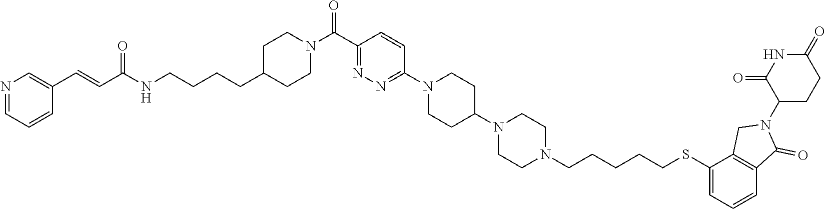 [SMILES: O=C(/C=C/c1cccnc1)NCCCCC1CCN(C(=O)c2ccc(N3CCC(N4CCN(CCCCCSc5cccc6c5CN(C5CCC(=O)NC5=O)C6=O)CC4)CC3)nn2)CC1] | (E)-N-(4-(1-(6-(4-(4-(5- ((2-(2,6-dioxopiperidin- 3-yl)-1-oxoisoindolin- 4-yl)thio)pentyl) piperazin- 1-yl)piperidin-1 - yl)pyridazine-3- carbonyl)piperidin-4- yl)butyl)-3-(pyridin- 3-yl)acrylamide | (E)-N-(4-(1-(6-(4-(4- (5-((2-(2,6- dioxopiperidin-3-yl)- 1-oxoisoindolin-4- yl)thio)pentyl) piperazin-1- yl)piperidin-1- yl)pyridazine-3- carbonyl)piperidin-4- yl)butyl)-3-(pyridin-3- yl)acrylamide |
| | 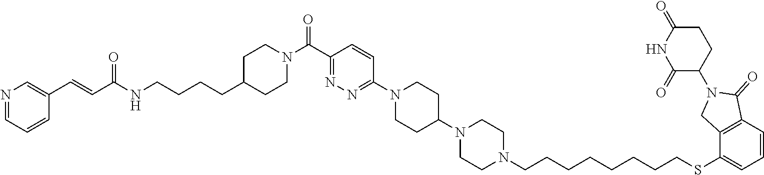 [SMILES: O=C(/C=C/c1cccnc1)NCCCCC1CCN(C(=O)c2ccc(N3CCC(N4CCN(CCCCCCCCSc5cccc6c5CN(C5CCC(=O)NC5=O)C6=O)CC4)CC3)nn2)CC1] | (E)-N-(4-(1-(6-(4-(4-(8- ((2-(2,6-dioxopiperidin- 3-yl)-1-oxoisoindolin- 4-yl)thio)octyl) piperazin- 1-yl)piperidin-1 - yl)pyridazine-3- carbonyl)piperidin-4- yl)butyl)-3-(pyridin-3- yl)acrylamide | (E)-N-(4-(1-(6-(4-(4- (8-((2-(2,6- dioxopiperidin-3-yl)- 1-oxoisoindolin-4- yl)thio)octyl) piperazin- 1-yl)piperidin-1- yl)pyridazine-3- carbonyl)piperidin-4- yl)butyl)-3-(pyridin- 3-yl)acrylamide |
| | 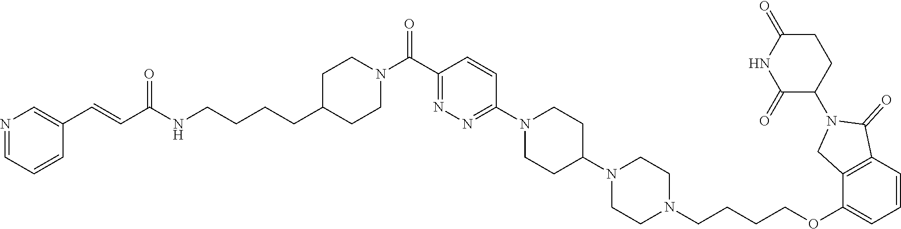 [SMILES: O=C(/C=C/c1cccnc1)NCCCCC1CCN(C(=O)c2ccc(N3CCC(N4CCN(CCCCOc5cccc6c5CN(C5CCC(=O)NC5=O)C6=O)CC4)CC3)nn2)CC1] | (E)-N-(4-(1-(6-(4-(4-(4- ((2-(2,6-dioxopiperidin- 3-yl)-1-oxoisoindolin- 4-yl)oxy)butyl) piperazin- 1-yl)piperidin-1- yl)pyridazine-3- carbonyl)piperidin-4- yl)butyl)-3-(pyridin- 3-yl)acrylamide | (E)-N-(4-(1-(6-(4-(4- (4-((2-(2,6- dioxopiperidin-3-yl)- 1-oxoisoindolin-4- yl)oxy)butyl) piperazin- 1-yl)piperidin-1 - yl)pyridazine-3- carbonyl)piperidin-4- yl)butyl)-3-(pyridin- 3-yl)acrylamide |
| | 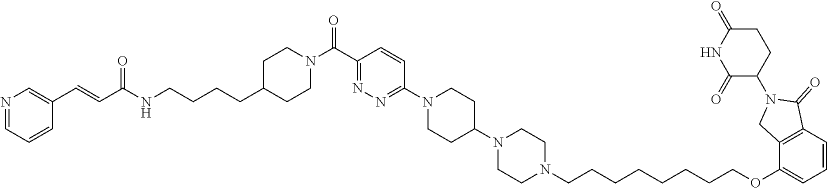 [SMILES: O=C(/C=C/c1cccnc1)NCCCCC1CCN(C(=O)c2ccc(N3CCC(N4CCN(CCCCCCCCOc5cccc6c5CN(C5CCC(=O)NC5=O)C6=O)CC4)CC3)nn2)CC1] | (E)-N-(4-(1-(6-(4-(4-(8- ((2-(2,6-dioxopiperidin- 3-yl)-1-oxoisoindolin- 4-yl)oxy)octyl) piperazin- 1-yl)piperidin-1- yl)pyridazine-3- carbonyl)piperidin-4- yl)butyl)-3-(pyridin- 3-yl)acrylamide | (E)-N-(4-(1-(6-(4-(4- (8-((2-(2,6- dioxopiperidin-3-yl)- 1-oxoisoindolin-4- yl)oxy)octyl) piperazin- 1-yl)piperidin-1- yl)pyridazine-3- carbonyl)piperidin-4- yl)butyl)-3-(pyridin- 3-yl)acrylamide |
| | 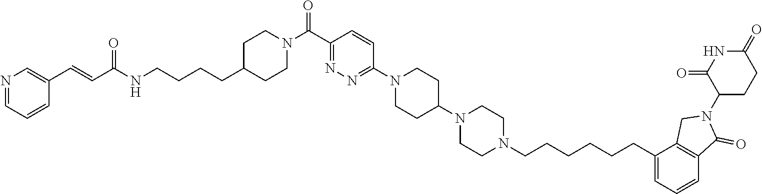 [SMILES: O=C(/C=C/c1cccnc1)NCCCCC1CCN(C(=O)c2ccc(N3CCC(N4CCN(CCCCCCc5cccc6c5CN(C5CCC(=O)NC5=O)C6=O)CC4)CC3)nn2)CC1] | (E)-N-(4-(1-(6-(4-(4-(6- (2-(2,6-dioxopiperidin- 3-yl)-1-oxoisoindolin- 4-yl)hexyl)piperazin- 1-yl)piperidin-1- yl)pyridazine-3- carbonyl)piperidin-4- yl)butyl)-3-(pyridin-3- yl)acrylamide | (E)-N-(4-(1-(6-(4-(4- (6-(2-(2,6- dioxopiperidin-3-yl)- 1-oxoisoindolin-4- yl)hexyl)piperazin- 1-yl)piperidin-1- yl)pyridazine-3- carbonyl)piperidin-4- yl)butyl)-3-(pyridin- 3-yl)acrylamide |
| | 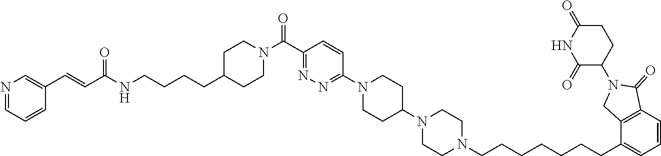 [SMILES: O=C(/C=C/c1cccnc1)NCCCCC1CCN(C(=O)c2ccc(N3CCC(N4CCN(CCCCCCCc5cccc6c5CN(C5CCC(=O)NC5=O)C6=O)CC4)CC3)nn2)CC1] | (E)-N-(4-(1-(6-(4-(4-(7- (2-(2,6-dioxopiperidin- 3-yl)-1-oxoisoindolin- 4-yl)heptyl)piperazin- 1-yl)piperidin-1- yl)pyridazine-3- carbonyl)piperidin-4- yl)butyl)-3-(pyridin-3- yl)acrylamide | (E)-N-(4-(1-(6-(4-(4- (7-(2-(2,6- dioxopiperidin-3-yl)- 1-oxoisoindolin-4- yl)heptyl)piperazin- 1-yl)piperidin-1 - yl)pyridazine-3- carbonyl)piperidin- 4-yl)butyl)-3- (pyridin-3- yl)acrylamide |
| | 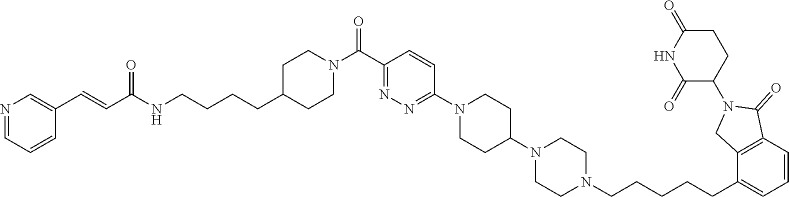 [SMILES: O=C(/C=C/c1cccnc1)NCCCCC1CCN(C(=O)c2ccc(N3CCC(N4CCN(CCCCCc5cccc6c5CN(C5CCC(=O)NC5=O)C6=O)CC4)CC3)nn2)CC1] | (E)-N-(4-(1-(6-(4-(4-(5- (2-(2,6-dioxopiperidin- 3-yl)-1-oxoisoindolin-4- yl)pentyl)piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (5-(2-(2,6- dioxopiperidin-3-yl)- 1-oxoisoindolin-4- yl)pentyl)piperazin- 1-yl)piperidin-1- yl)pyridazine-3- carbonyl)piperidin-4- yl)butyl)-3-(pyridin-3- yl)acrylamide |
| | 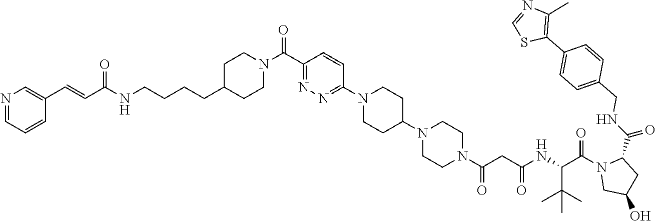 [SMILES: Cc1ncsc1-c1ccc(CNC(=O)[C@@H]2C[C@@H](O)CN2C(=O)[C@@H](NC(=O)CC(=O)N2CCN(C3CCN(c4ccc(C(=O)N5CCC(CCCCNC(=O)/C=C/c6cccnc6)CC5)nn4)CC3)CC2)C(C)(C)C)cc1] | (2S,4R)-1-((S)-3,3- dimethyl-2-(3-oxo-3- (4-(1-(6-(4-(4-((E)-3- (pyridin-3- yl)acrylamido)butyl) piperidine-1- carbonyl)pyridazin-3- yl)piperidin-4- yl)piperazin-1- yl)propanamido) butanoyl)-4- hydroxy-N-(4-(4- methylthiazol-5- yl)benzyl)pyrrolidine- 2-carboxamide | (2S,4R)-1-((S)-3,3- dimethyl-2-(3-oxo-3- (4-(1-(6-(4-(4-((E)-3- (pyridin-3- yl)acrylamido)butyl) piperidine-1- carbonyl)pyridazin- 3-yl)piperidin-4- yl)piperazin-1- yl)propanamido) butanoyl)-4- hydroxy-N-(4-(4- methylthiazol-5- yl)benzyl)pyrrolidine- 2-carboxamide |
| | 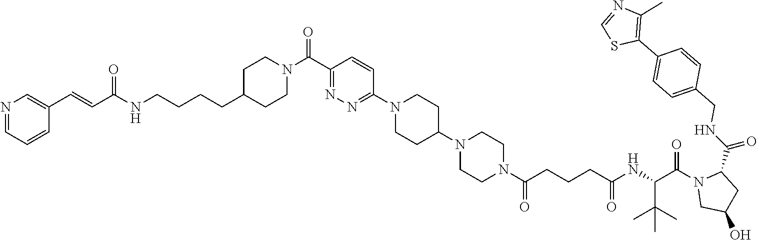 [SMILES: Cc1ncsc1-c1ccc(CNC(=O)[C@@H]2C[C@@H](O)CN2C(=O)[C@@H](NC(=O)CCCC(=O)N2CCN(C3CCN(c4ccc(C(=O)N5CCC(CCCCNC(=O)/C=C/c6cccnc6)CC5)nn4)CC3)CC2)C(C)(C)C)cc1] | (2S,4R)-1-((S)-3,3- dimethyl-2-(5-oxo-5- (4-(1-(6-(4-(4-((E)-3- (pyridin-3- yl)acrylamido)butyl) piperidine-1- carbonyl)pyridazin- 3-yl)piperidin-4- yl)piperazin-1- yl)pentanamido) butanoyl)-4- hydroxy-N-(4-(4- methylthiazol-5- yl)benzyl)pyrrolidine- 2-carboxamide | (2S,4R)-1-((S)-3,3- dimethyl-2-(5-oxo-5- (4-(1-(6-(4-(4-((E)-3- (pyridin-3- yl)acrylamido)butyl) piperidine-1- carbonyl)pyridazin- 3-yl)piperidin-4- yl)piperazin-1- yl)pentanamido) butanoyl)-4- hydroxy-N-(4-(4- methylthiazol-5- yl)benzyl)pyrrolidine- 2-carboxamide |
| | 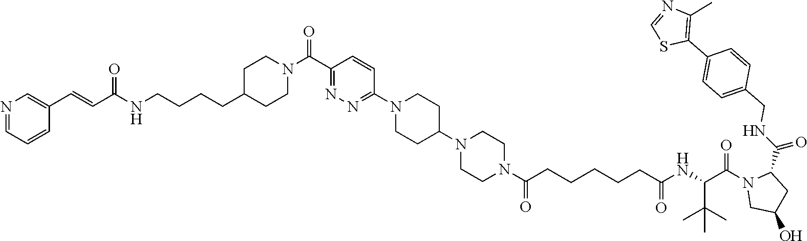 [SMILES: Cc1ncsc1-c1ccc(CNC(=O)[C@@H]2C[C@@H](O)CN2C(=O)[C@@H](NC(=O)CCCCCC(=O)N2CCN(C3CCN(c4ccc(C(=O)N5CCC(CCCCNC(=O)/C=C/c6cccnc6)CC5)nn4)CC3)CC2)C(C)(C)C)cc1] | (2S,4R)-1-((S)-3,3- dimethyl-2-(7-oxo- 7-(4-(1-(6-(4-(4- ((E)-3-(pyridin-3- yl)acrylamido)butyl) piperidine-1- carbonyl)pyridazin- 3-yl)piperidin-4- yl)piperazin-1- yl)heptanamido) butanoyl)-4- hydroxy-N-(4-(4- methylthiazol-5- yl)benzyl)pyrrolidine- 2-carboxamide | (2S,4R)-1-((S)-3,3- dimethyl-2-(7-oxo- 7-(4-(1-(6-(4-(4- ((E)-3-(pyridin-3- yl)acrylamido)butyl) piperidine-1- carbonyl)pyridazin- 3-yl)piperidin-4- yl)piperazin-1- yl)heptanamido) butanoyl)-4- hydroxy-N-(4-(4- methylthiazol-5- yl)benzyl)pyrrolidine- 2-carboxamide |
| | 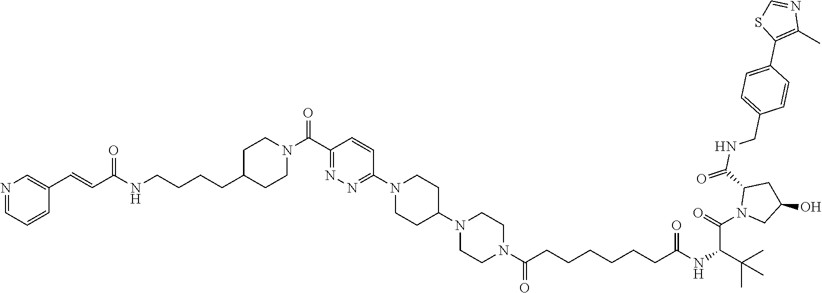 [SMILES: Cc1ncsc1-c1ccc(CNC(=O)[C@@H]2C[C@@H](O)CN2C(=O)[C@@H](NC(=O)CCCCCCC(=O)N2CCN(C3CCN(c4ccc(C(=O)N5CCC(CCCCNC(=O)/C=C/c6cccnc6)CC5)nn4)CC3)CC2)C(C)(C)C)cc1] | (2S,4R)-1-((S)-3,3- dimethyl-2-(8-oxo- 8-(4-(1-(6-(4-(4- ((E)-3-(pyridin-3- yl)acrylamido)butyl) piperidine-1- carbonyl)pyridazin- 3-yl)piperidin-4- yl)piperazin-1- yl)octanamido) butanoyl)-4- hydroxy-N-(4-(4- methylthiazol-5- yl)benzyl)pyrrolidine- 2-carboxamide | (2S,4R)-1-((S)-3,3- dimethyl-2-(8-oxo- 8-(4-(1-(6-(4-(4- ((E)-3-(pyridin-3- yl)acrylamido)butyl) piperidine-1- carbonyl)pyridazin- 3-yl)piperidin-4- yl)piperazin-1- yl)octanamido) butanoyl)-4- hydroxy-N-(4-(4- methylthiazol-5- yl)benzyl)pyrrolidine- 2-carboxamide |
| | 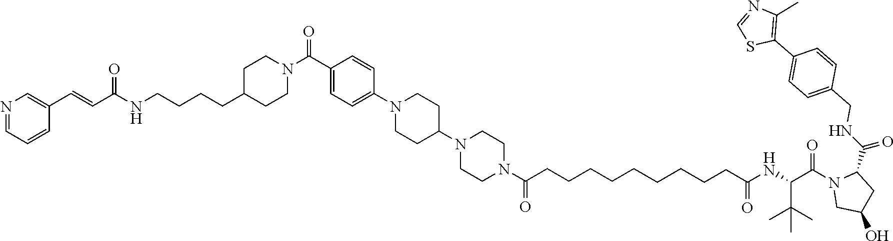 [SMILES: Cc1ncsc1-c1ccc(CNC(=O)[C@@H]2C[C@@H](O)CN2C(=O)[C@@H](NC(=O)CCCCCCCCCC(=O)N2CCN(C3CCN(c4ccc(C(=O)N5CCC(CCCCNC(=O)/C=C/c6cccnc6)CC5)cc4)CC3)CC2)C(C)(C)C)cc1] | (2S,4R)-1-((S)-3,3- dimethyl-2-(11-oxo- 11-(4-(1-(6-(4-(4- ((E)-3-(pyridin-3- yl)acrylamido)butyl) piperidine-1- carbonyl)pyridazin- 3-yl)piperidin-4- yl)piperazin-1- yl)undecanamido) butanoyl)-4- hydroxy-N-(4-(4- methylthiazol-5- yl)benzyl)pyrrolidine-2- carboxamide | (2S,4R)-1-((S)-3,3- dimethyl-2-(11-oxo- 11-(4-(1-(6-(4-(4- ((E)-3-(pyridin-3- yl)acrylamido)butyl) piperidine-1- carbonyl)pyridazin- 3-yl)piperidin-4- yl)piperazin-1- yl)undecanamido) butanoyl)-4- hydroxy-N-(4- (4-methylthiazol-5- yl)benzyl)pyrrolidine- 2-carboxamide |
| | 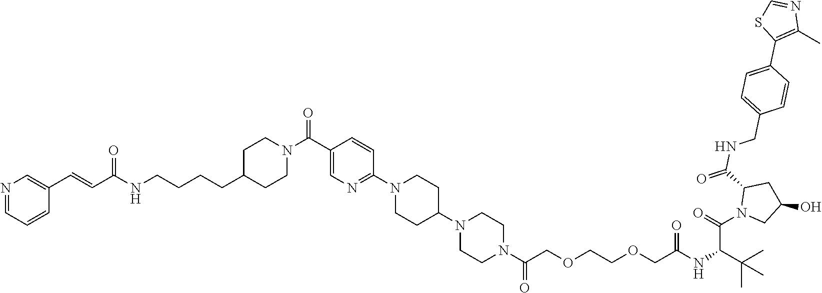 [SMILES: Cc1ncsc1-c1ccc(CNC(=O)[C@@H]2C[C@@H](O)CN2C(=O)[C@@H](NC(=O)COCCOCC(=O)N2CCN(C3CCN(c4ccc(C(=O)N5CCC(CCCCNC(=O)/C=C/c6cccnc6)CC5)cn4)CC3)CC2)C(C)(C)C)cc1] | (2S,4R)-1-((S)-3,3- dimethyl-2-(2-(2-(2- ox0-2-(4-(1-(6-(4-(4- ((E)-3-(pyridin-3- yl)acrylamido)butyl) piperidine-1- carbonyl)pyridazin- 3-yl)piperidin-4- yl)piperazin-1- yl)ethoxy)ethoxy) acetamido)butanoyl)- 4-hydroxy-N-(4-(4- methylthiazol-5- yl)benzyl) pyrrolidine-2- carboxamide | (2S,4R)-1-((S)-3,3- dimethyl-2-(2-(2-(2- ox0-2-(4-(1-(6-(4-(4- ((E)-3-(pyridin-3- yl)acrylamido)butyl) piperidine-1- carbonyl)pyridazin- 3-yl)piperidin-4- yl)piperazin-1- yl)ethoxy)ethoxy) acetamido)butanoyl)- 4-hydroxy-N-(4-(4- methylthiazol-5- yl)benzyl) pyrrolidine- 2-carboxamide |
| | 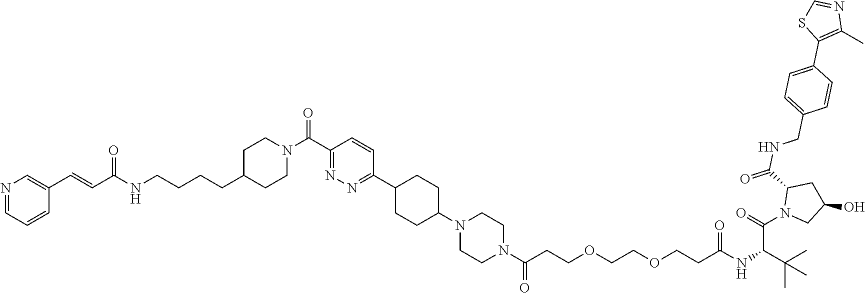 [SMILES: Cc1ncsc1-c1ccc(CNC(=O)[C@@H]2C[C@@H](O)CN2C(=O)[C@@H](NC(=O)CCOCCOCCC(=O)N2CCN(C3CCC(c4ccc(C(=O)N5CCC(CCCCNC(=O)/C=C/c6cccnc6)CC5)nn4)CC3)CC2)C(C)(C)C)cc1] | (2S,4R)-1-((S)-3,3- dimethyl-2-(3-(2-(3- oxo-3-(4-(1-(6-(4-(4- ((E)-3-(pyridin-3- yl)acrylamido)butyl) piperidine-1- carbonyl)pyridazin- 3-yl)piperidin-4- yl)piperazin-1- yl)propoxy)ethoxy) propanamido) butanoyl)-4- hydroxy-N-(4-(4- methylthiazol-5- yl)benzyl)pyrrolidine- 2-carboxamide | (2S,4R)-1-((S)-3,3- dimethyl-2-(3-(2-(3- ox0-3-(4-(1-(6-(4-(4- ((E)-3-(pyridin-3- yl)acrylamido)butyl) piperidine-1- carbonyl)pyridazin- 3-yl)piperidin-4- yl)piperazin-1- yl)propoxy)ethoxy) propanamido) butanoyl)-4- hydroxy-N-(4-(4- methylthiazol-5- yl)benzyl)pyrrolidine- 2-carboxamide |
| | 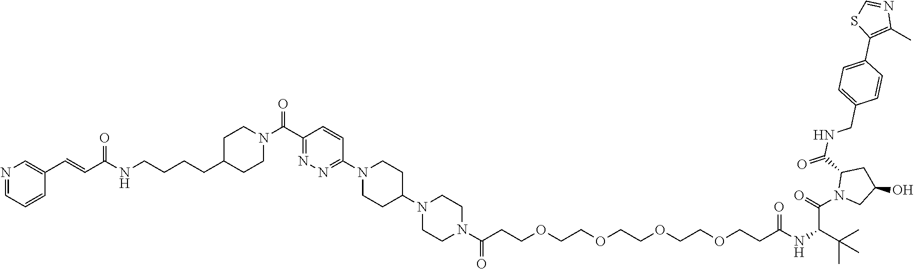 [SMILES: Cc1ncsc1-c1ccc(CNC(=O)[C@@H]2C[C@@H](O)CN2C(=O)[C@@H](NC(=O)CCOCCOCCOCCOCCC(=O)N2CCN(C3CCN(c4ccc(C(=O)N5CCC(CCCCNC(=O)/C=C/c6cccnc6)CC5)nn4)CC3)CC2)C(C)(C)C)cc1] | (2S,4R)-1-((S)-2-(tert- butyl)-4, 19-dioxo-19- (4-(1-(6-(4-(4-((E)-3- (pyridin-3- yl)acrylamido)butyl) piperidine-1- carbonyl)pyridazin-3- yl)piperidin-4- yl)piperazin-1-yl)- 7,10,13,16-tetraoxa-3- azanonadecanoyl)-4- hydroxy-N-(4-(4- methylthiazol-5- yl)benzyl)pyrrolidine- 2-carboxamide | (2S,4R)-1-((S)-2-(tert- butyl)-4,19-dioxo-19- (4-(1-(6-(4-(4-((E)-3- (pyridin-3- yl)acrylamido)butyl) piperidine-1- carbonyl)pyridazin-3- yl)piperidin-4- yl)piperazin-1-yl)- 7,10,13,16-tetraoxa-3- azanonadecanoyl)-4- hydroxy-N-(4-(4- methylthiazol-5- yl)benzyl)pyrrolidine- 2-carboxamide |
| | 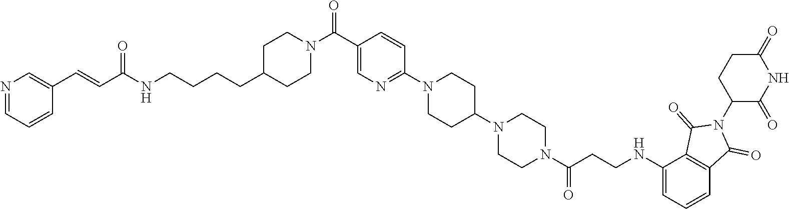 [SMILES: O=C(/C=C/c1cccnc1)NCCCCC1CCN(C(=O)c2ccc(N3CCC(N4CCN(C(=O)CCNc5cccc6c5C(=O)N(C5CCC(=O)NC5=O)C6=O)CC4)CC3)nc2)CC1] | (E)-N-(4-(1-(6-(4-(4-(3- ((2-(2,6-dioxopiperidin- 3-yl)-1,3- dioxoisoindolin-4- yl)amino)propanoyl) piperazin-1-yl) piperidin-1- yl)nicotinoyl)piperidin- 4-yl)butyl)-3-(pyridin- 3-yl)acrylamide | (E)-N-(4-(1-(6-(4-(4- (3-((2-(2,6- dioxopiperidin-3-yl)- 1,3-dioxoisoindolin- 4-yl)amino) propanoyl) piperazin-1- yl)piperidin-1- yl)nicotinoyl) piperidin-4- yl)butyl)- 3-(pyridin- 3-yl)acrylamide |
| | 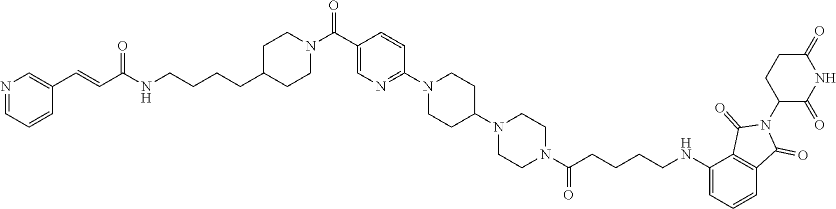 [SMILES: O=C(/C=C/c1cccnc1)NCCCCC1CCN(C(=O)c2ccc(N3CCC(N4CCN(C(=O)CCCCNc5cccc6c5C(=O)N(C5CCC(=O)NC5=O)C6=O)CC4)CC3)nc2)CC1] | (E)-N-(4-(1-(6-(4-(4-(5- ((2-(2,6-dioxopiperidin- 3-yl)-1,3- dioxoisoindolin-4- yl)amino)pentanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (5-((2-(2,6- dioxopiperidin-3-yl)- 1,3-dioxoisoindolin- 4-yl)amino)pentanoyl) piperazin-1- yl)piperidin-1- yl)nicotinoyl)piperidin- 4-yl)butyl)-3-(pyridin- 3-yl)acrylamide |
| | 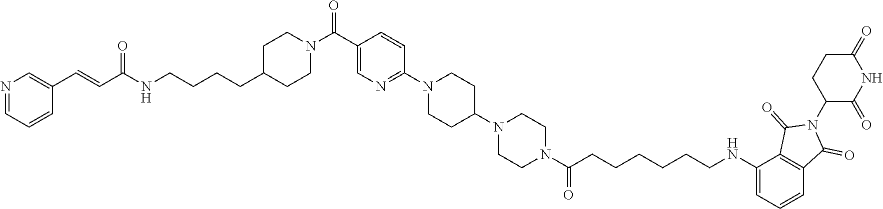 [SMILES: O=C(/C=C/c1cccnc1)NCCCCC1CCN(C(=O)c2ccc(N3CCC(N4CCN(C(=O)CCCCCCNc5cccc6c5C(=O)N(C5CCC(=O)NC5=O)C6=O)CC4)CC3)nc2)CC1] | (E)-N-(4-(1-(6-(4-(4-(7- ((2-(2,6-dioxopiperidin- 3-yl)-1,3- dioxoisoindolin-4- yl)amino)heptanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (7-((2-(2,6- dioxopiperidin-3-yl)- 1,3-dioxoisoindolin- 4-yl)amino) heptanoyl) piperazin-1- yl)piperidin-1- yl)nicotinoyl) piperidin-4- yl)butyl)-3-(pyridin- 3-yl)acrylamide |
| | 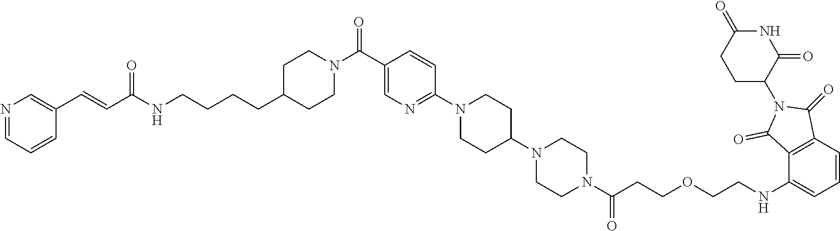 [SMILES: O=C(/C=C/c1cccnc1)NCCCCC1CCN(C(=O)c2ccc(N3CCC(N4CCN(C(=O)CCOCCNc5cccc6c5C(=O)N(C5CCC(=O)NC5=O)C6=O)CC4)CC3)nc2)CC1] | (E)-N-(4-(1-(6-(4-(4-(3- (2-((2-(2,6- dioxopiperidin-3-yl)- 1,3-dioxoisoindolin- 4-yl)amino)ethoxy) propanoyl)piperazin- 1-yl)piperidin-1- yl)nicotinoyl)piperidin- 4-yl)butyl)-3-(pyridin- 3-yl)acrylamide | (E)-N-(4-(1-(6-(4-(4- (3-(2-((2-(2,6- dioxopiperidin-3-yl)- 1,3-dioxoisoindolin- 4-yl)amino)ethoxy) propanoyl)piperazin- 1-yl)piperidin-1- yl)nicotinoyl)piperidin- 4-yl)butyl)-3-(pyridin- 3-yl)acrylamide |
| | 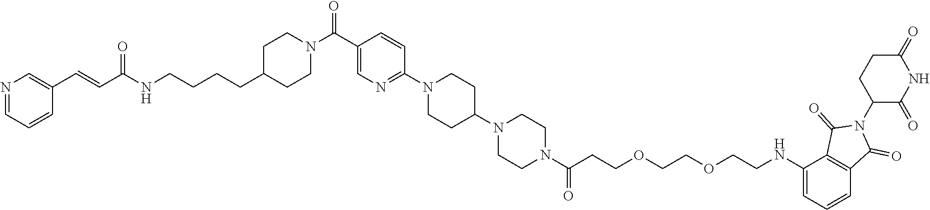 [SMILES: O=C(/C=C/c1cccnc1)NCCCCC1CCN(C(=O)c2ccc(N3CCC(N4CCN(C(=O)CCOCCOCCNc5cccc6c5C(=O)N(C5CCC(=O)NC5=O)C6=O)CC4)CC3)nc2)CC1] | (E)-N-(4-(1-(6-(4-(4-(3- (2-(2-((2-(2,6- dioxopiperidin-3-yl)- 1,3-dioxoisoindolin- 4-yl)amino)ethoxy) ethoxy)propanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (3-(2-(2-((2-(2,6- dioxopiperidin-3-yl)- 1,3-dioxoisoindolin- 4-yl)amino)ethoxy) ethoxy)propanoyl) piperazin- 1-yl)piperidin-1- yl)nicotinoyl)piperidin- 4-yl)butyl)-3-(pyridin- 3-yl)acrylamide |
| | 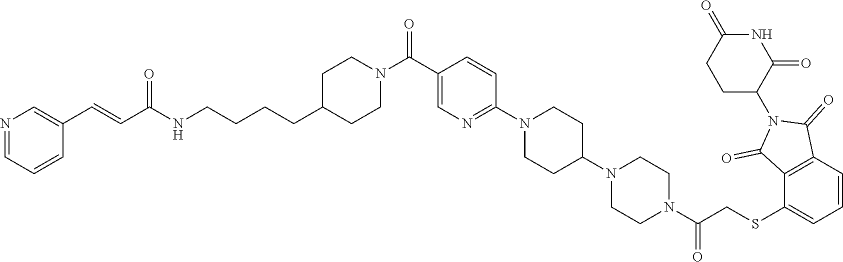 [SMILES: O=C(/C=C/c1cccnc1)NCCCCC1CCN(C(=O)c2ccc(N3CCC(N4CCN(C(=O)CSc5cccc6c5C(=O)N(C5CCC(=O)NC5=O)C6=O)CC4)CC3)nc2)CC1] | (E)-N-(4-(1-(6-(4-(4-(2- ((2-(2,6-dioxopiperidin- 3-yl)-1,3- dioxoisoindolin-4- yl)thio)acetyl) piperazin- 1-yl)piperidin-1- yl)nicotinoyl)piperidin- 4-yl)butyl)-3-(pyridin- 3-yl)acrylamide | (E)-N-(4-(1-(6-(4-(4- (2-((2-(2,6- dioxopiperidin-3-yl)- 1,3-dioxoisoindolin- 4-yl)thio)acetyl) piperazin-1- yl)piperidin-1- yl)nicotinoyl)piperidin- 4-yl)butyl)-3-(pyridin- 3-yl)acrylamide |
| | 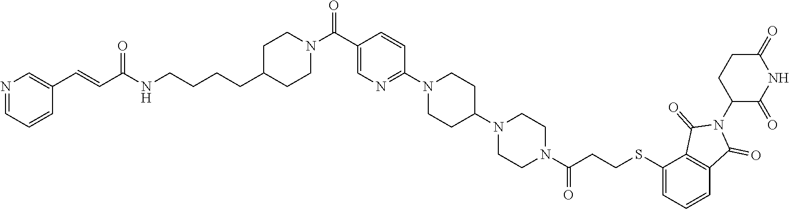 [SMILES: O=C(/C=C/c1cccnc1)NCCCCC1CCN(C(=O)c2ccc(N3CCC(N4CCN(C(=O)CCSc5cccc6c5C(=O)N(C5CCC(=O)NC5=O)C6=O)CC4)CC3)nc2)CC1] | (E)-N-(4-(1-(6-(4-(4-(3- ((2-(2,6-dioxopiperidin- 3-yl)-1,3- dioxoisoindolin-4- yl)thio)propanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (3-((2-(2,6- dioxopiperidin-3-yl)- 1,3-dioxoisoindolin- 4-yl)thio)propanoyl) piperazin-1- yl)piperidin-1- yl)nicotinoyl)piperidin- 4-yl)butyl)-3-(pyridin- 3-yl)acrylamide |
| | 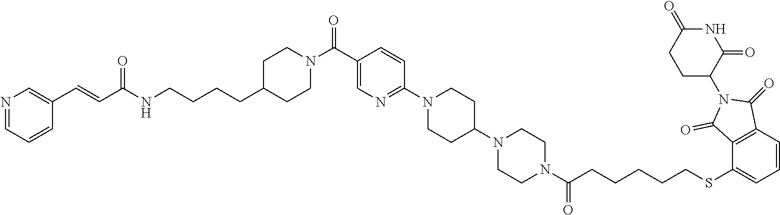 [SMILES: O=C(/C=C/c1cccnc1)NCCCCC1CCN(C(=O)c2ccc(N3CCC(N4CCN(C(=O)CCCCCSc5cccc6c5C(=O)N(C5CCC(=O)NC5=O)C6=O)CC4)CC3)nc2)CC1] | (E)-N-(4-(1-(6-(4-(4-(6- ((2-(2,6-dioxopiperidin- 3-yl)-1,3- dioxoisoindolin-4- yl)thio)hexanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (6-((2-(2,6- dioxopiperidin-3-yl)- 1,3-dioxoisoindolin- 4-yl)thio)hexanoyl) piperazin-1- yl)piperidin-1- yl)nicotinoyl) piperidin-4- yl)butyl)-3-(pyridin- 3-yl)acrylamide |
| | 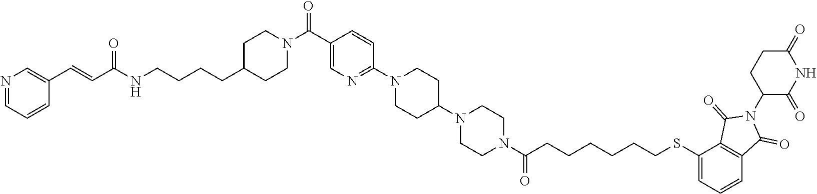 [SMILES: O=C(/C=C/c1cccnc1)NCCCCC1CCN(C(=O)c2ccc(N3CCC(N4CCN(C(=O)CCCCCCSc5cccc6c5C(=O)N(C5CCC(=O)NC5=O)C6=O)CC4)CC3)nc2)CC1] | (E)-N-(4-(1-(6-(4-(4-(7- ((2-(2,6-dioxopiperidin- 3-yl)-1,3- dioxoisoindolin-4- yl)thio)heptanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (7-((2-(2,6- dioxopiperidin-3-yl)- 1,3-dioxoisoindolin-4- yl)thio)heptanoyl) piperazin-1- yl)piperidin-1- yl)nicotinoyl)piperidin- 4-yl)butyl)-3-(pyridin- 3-yl)acrylamide |
| | 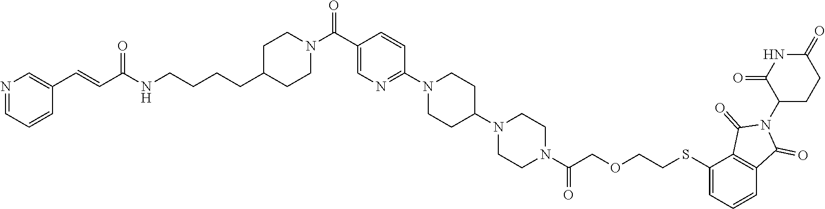 [SMILES: O=C(/C=C/c1cccnc1)NCCCCC1CCN(C(=O)c2ccc(N3CCC(N4CCN(C(=O)COCCSc5cccc6c5C(=O)N(C5CCC(=O)NC5=O)C6=O)CC4)CC3)nc2)CC1] | (E)-N-(4-(1-(6-(4-(4-(2- (2-((2-(2,6- dioxopiperidin-3-yl)- 1,3-dioxoisoindolin-4- yl)thio)ethoxy)acetyl) piperazin-1- yl)piperidin-1- yl)nicotinoyl)piperidin- 4-yl)butyl)-3-(pyridin-3- yl)acrylamide | (E)-N-(4-(1-(6-(4-(4- (2-(2-((2-(2,6- dioxopiperidin-3-yl)- 1,3-dioxoisoindolin-4- yl)thio)ethoxy)acetyl) piperazin-1- yl)piperidin-1- yl)nicotinoyl)piperidin- 4-yl)butyl)-3-(pyridin- 3-yl)acrylamide |
| | 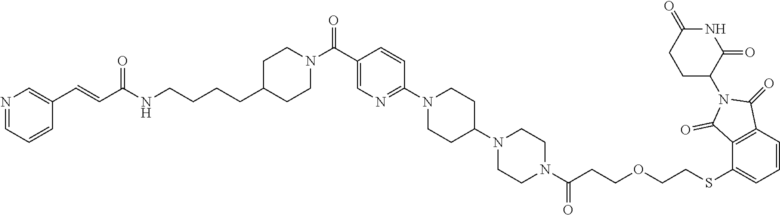 [SMILES: O=C(/C=C/c1cccnc1)NCCCCC1CCN(C(=O)c2ccc(N3CCC(N4CCN(C(=O)CCOCCSc5cccc6c5C(=O)N(C5CCC(=O)NC5=O)C6=O)CC4)CC3)nc2)CC1] | (E)-N-(4-(1-(6-(4-(4-(3- (2-((2-(2,6- dioxopiperidin-3-yl)- 1,3-dioxoisoindolin- 4-yl)thio)ethoxy) propanoyl)piperazin- 1-yl)piperidin-1- yl)nicotinoyl)piperidin- 4-yl)butyl)-3-(pyridin- 3-yl)acrylamide | (E)-N-(4-(1-(6-(4-(4- (3-(2-((2-(2,6- dioxopiperidin-3-yl)- 1,3-dioxoisoindolin- 4-yl)thio)ethoxy) propanoyl)piperazin- 1-yl)piperidin-1- yl)nicotinoyl)piperidin- 4-yl)butyl)-3-(pyridin- 3-yl)acrylamide |
| | 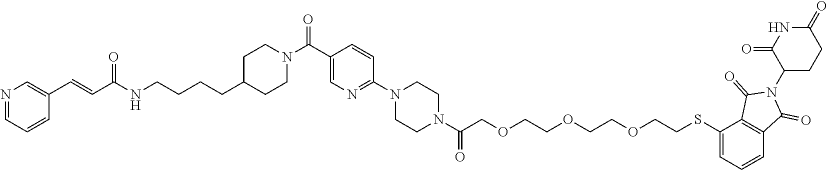 [SMILES: O=C(/C=C/c1cccnc1)NCCCCC1CCN(C(=O)c2ccc(N3CCN(C(=O)COCCOCCOCCSc4cccc5c4C(=O)N(C4CCC(=O)NC4=O)C5=O)CC3)nc2)CC1] | (E)-N-(4-(1-(6-(4-(4-(2- (2-(2-(2-((2-(2,6- dioxopiperidin-3-yl)- 1,3-dioxoisoindolin-4- yl)thio)ethoxy)ethoxy) ethoxy)acetyl) piperazin-1- yl)piperidin-1- yl)nicotinoyl)piperidin- 4-yl)butyl)-3-(pyridin- 3-yl)acrylamide | (E)-N-(4-(1-(6-(4-(4- (2-(2-(2-(2-((2-(2,6- dioxopiperidin-3-yl)- 1,3-dioxoisoindolin- 4-yl)thio)ethoxy) ethoxy)ethoxy)acetyl) piperazin- 1-yl)piperidin-1- yl)nicotinoyl)piperidin- 4-yl)butyl)-3-(pyridin- 3-yl)acrylamide |
| | 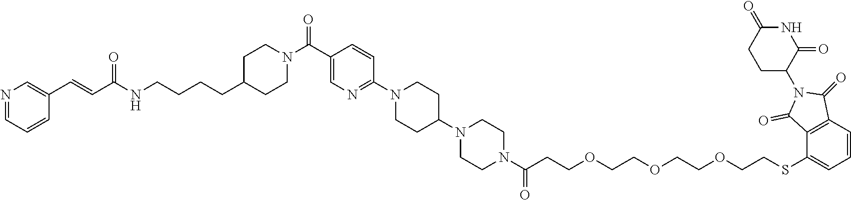 [SMILES: O=C(/C=C/c1cccnc1)NCCCCC1CCN(C(=O)c2ccc(N3CCC(N4CCN(C(=O)CCOCCOCCOCCSc5cccc6c5C(=O)N(C5CCC(=O)NC5=O)C6=O)CC4)CC3)nc2)CC1] | (E)-N-(4-(1-(6-(4-(4-(3- (2-(2-(2-((2-(2,6- dioxopiperidin-3-yl)- 1,3-dioxoisoindolin-4- yl)thio)ethoxy)ethoxy) ethoxy)propanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (3-(2-(2-(2-((2-(2,6- dioxopiperidin-3-yl)- 1,3-dioxoisoindolin-4- yl)thio)ethoxy)ethoxy) ethoxy)propanoyl) piperazin-1- yl)piperidin-1- yl)nicotinoyl)piperidin- 4-yl)butyl)-3-(pyridin- 3-yl)acrylamide |
| | 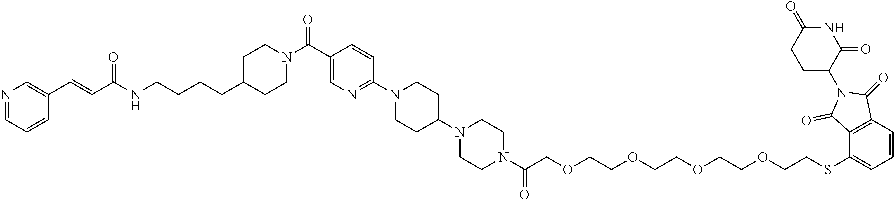 [SMILES: O=C(/C=C/c1cccnc1)NCCCCC1CCN(C(=O)c2ccc(N3CCC(N4CCN(C(=O)COCCOCCOCCOCCSc5cccc6c5C(=O)N(C5CCC(=O)NC5=O)C6=O)CC4)CC3)nc2)CC1] | (E)-N-(4-(1-(6-(4-(4- (14-((2-(2,6- dioxopiperidin-3-yl)- 1,3-dioxoisoindolin- 4-yl)thio)-3,6,9,12- tetraoxatetradecanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (14-((2-(2,6- dioxopiperidin-3-yl)- 1,3-dioxoisoindolin- 4-yl)thio)-3,6,9,12- tetraoxatetradecanoyl) piperazin-1- yl)piperidin-1- yl)nicotinoyl)piperidin- 4-yl)butyl)-3-(pyridin- 3-yl)acrylamide |
| | 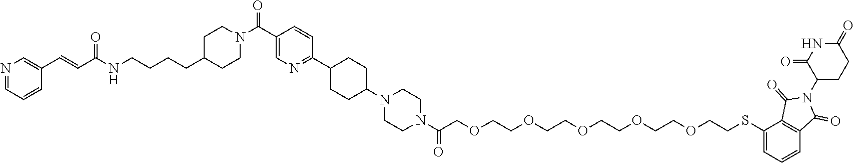 [SMILES: O=C(/C=C/c1cccnc1)NCCCCC1CCN(C(=O)c2ccc(C3CCC(N4CCN(C(=O)COCCOCCOCCOCCOCCSc5cccc6c5C(=O)N(C5CCC(=O)NC5=O)C6=O)CC4)CC3)nc2)CC1] | (E)-N-(4-(1-(6-(4-(4- (17-((2-(2,6- dioxopiperidin-3-yl)- 1,3-dioxoisoindolin-4- yl)thio)-3,6,9,12,15- pentaoxaheptadecanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (17-((2-(2,6- dioxopiperidin-3-yl)- 1,3-dioxoisoindolin- 4-yl)thio)-3,6,9,12,15- pentaoxaheptadecanoyl) piperazin-1- yl)piperidin-1- yl)nicotinoyl)piperidin- 4-yl)butyl)-3-(pyridin- 3-yl)acrylamide |
| | 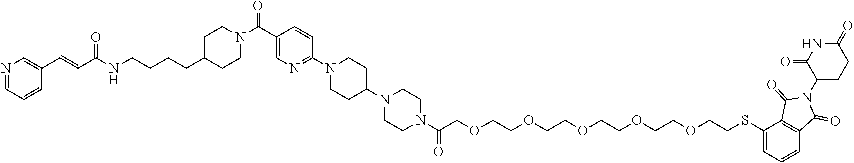 [SMILES: O=C(/C=C/c1cccnc1)NCCCCC1CCN(C(=O)c2ccc(N3CCC(N4CCN(C(=O)COCCOCCOCCOCCOCCSc5cccc6c5C(=O)N(C5CCC(=O)NC5=O)C6=O)CC4)CC3)nc2)CC1] | (E)-N-(4-(1-(6-(4-(4-(2- ((2-(2,6-dioxopiperidin- 3-yl)-1,3- dioxoisoindolin-4- yl)oxy)acetyl)piperazin- 1-yl)piperidin-1- yl)nicotinoyl)piperidin- 4-yl)butyl)-3-(pyridin- 3-yl)acrylamide | (E)-N-(4-(1-(6-(4-(4- (2-((2-(2,6- dioxopiperidin-3-yl)- 1,3-dioxoisoindolin- 4-yl)oxy)acetyl) piperazin-1- yl)piperidin-1- yl)nicotinoyl)piperidin- 4-yl)butyl)-3-(pyridin- 3-yl)acrylamide |
| | 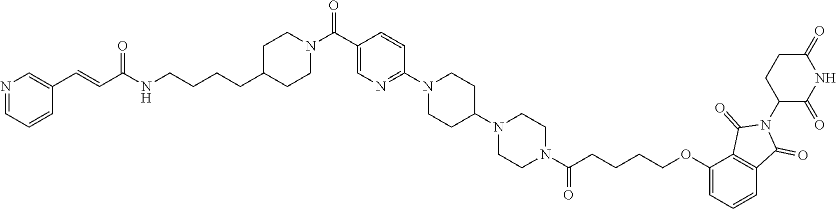 [SMILES: O=C(/C=C/c1cccnc1)NCCCCC1CCN(C(=O)c2ccc(N3CCC(N4CCN(C(=O)CCCCOc5cccc6c5C(=O)N(C5CCC(=O)NC5=O)C6=O)CC4)CC3)nc2)CC1] | (E)-N-(4-(1-(6-(4-(4-(5- ((2-(2,6-dioxopiperidin- 3-yl)-1,3- dioxoisoindolin-4- yl)oxy)pentanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (5-((2-(2,6- dioxopiperidin-3-yl)- 1,3-dioxoisoindolin- 4-yl)oxy)pentanoyl) piperazin-1- yl)piperidin-1- yl)nicotinoyl) piperidin-4- yl)butyl)-3-(pyridin- 3-yl)acrylamide |
| | 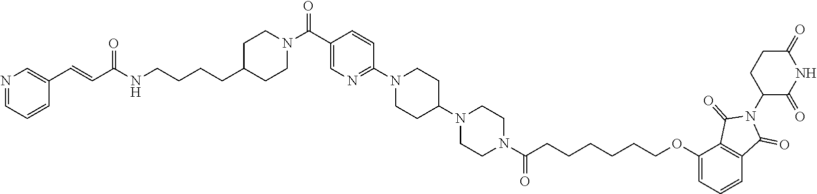 [SMILES: O=C(/C=C/c1cccnc1)NCCCCC1CCN(C(=O)c2ccc(N3CCC(N4CCN(C(=O)CCCCCCOc5cccc6c5C(=O)N(C5CCC(=O)NC5=O)C6=O)CC4)CC3)nc2)CC1] | (E)-N-(4-(1-(6-(4-(4-(7- ((2-(2,6-dioxopiperidin- 3-yl)-1,3- dioxoisoindolin-4- yl)oxy)heptanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (7-((2-(2,6- dioxopiperidin-3-yl)- 1,3-dioxoisoindolin- 4-yl)oxy)heptanoyl) piperazin-1- yl)piperidin-1- yl)nicotinoyl) piperidin-4- yl)butyl)-3-(pyridin- 3-yl)acrylamide |
| | 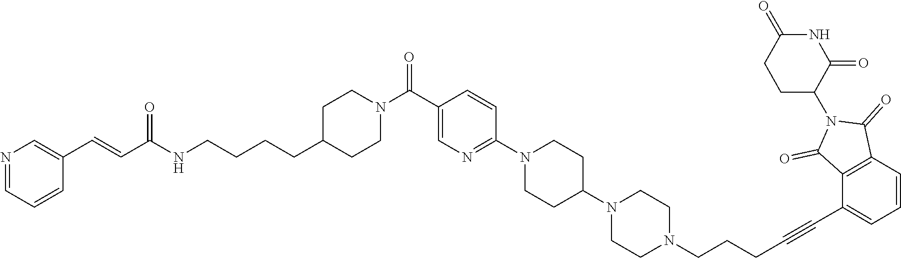 [SMILES: O=C(/C=C/c1cccnc1)NCCCCC1CCN(C(=O)c2ccc(N3CCC(N4CCN(CCCC#Cc5cccc6c5C(=O)N(C5CCC(=O)NC5=O)C6=O)CC4)CC3)nc2)CC1] | (E)-N-(4-(1-(6-(4-(4-(5- (2-(2,6-dioxopiperidin- 3-yl)-1,3- dioxoisoindolin-4- yl)pent-4-yn-1- yl)piperazin-1- yl)piperidin-1- yl)nicotinoyl)piperidin- 4-yl)butyl)-3-(pyridin- 3-yl)acrylamide | (E)-N-(4-(1-(6-(4-(4- (5-(2-(2,6- dioxopiperidin-3-yl)- 1,3-dioxoisoindolin- 4-yl)pent-4-yn-1- yl)piperazin-1- yl)piperidin-1- yl)nicotinoyl)piperidin- 4-yl)butyl)-3-(pyridin- 3-yl)acrylamide |
| | 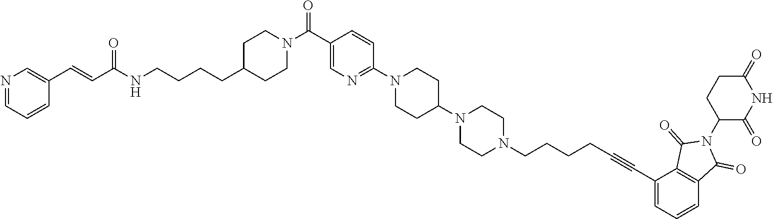 [SMILES: O=C(/C=C/c1cccnc1)NCCCCC1CCN(C(=O)c2ccc(N3CCC(N4CCN(CCCCC#Cc5cccc6c5C(=O)N(C5CCC(=O)NC5=O)C6=O)CC4)CC3)nc2)CC1] | (E)-N-(4-(1-(6-(4-(4-(6- (2-(2,6-dioxopiperidin- 3-yl)-1,3- dioxoisoindolin-4- yl)hex-5-yn-1- yl)piperazin-1- yl)piperidin-1- yl)nicotinoyl)piperidin- 4-yl)butyl)-3-(pyridin- 3-yl)acrylamide | (E)-N-(4-(1-(6-(4-(4- (6-(2-(2,6- dioxopiperidin-3-yl)- 1,3-dioxoisoindolin- 4-yl)hex-5-yn-1- yl)piperazin-1- yl)piperidin-1- yl)nicotinoyl)piperidin- 4-yl)butyl)-3-(pyridin- 3-yl)acrylamide |
| | 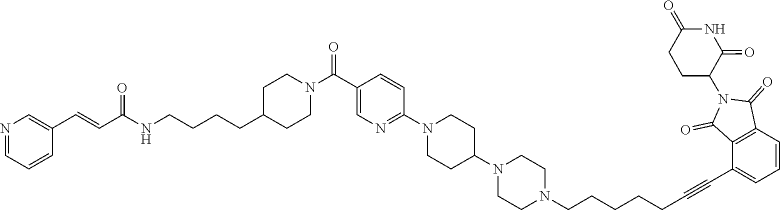 [SMILES: O=C(/C=C/c1cccnc1)NCCCCC1CCN(C(=O)c2ccc(N3CCC(N4CCN(CCCCCC#Cc5cccc6c5C(=O)N(C5CCC(=O)NC5=O)C6=O)CC4)CC3)nc2)CC1] | (E)-N-(4-(1-(6-(4-(4-(7- (2-(2,6-dioxopiperidin- 3-yl)-1,3- dioxoisoindolin-4- yl)hept-6-yn-1- yl)piperazin-1- yl)piperidin-1- yl)nicotinoyl)piperidin- 4-yl)butyl)-3-(pyridin- 3-yl)acrylamide | (E)-N-(4-(1-(6-(4-(4- (7-(2-(2,6- dioxopiperidin-3-yl)- 1,3-dioxoisoindolin- 4-yl)hept-6-yn-1- yl)piperazin-1- yl)piperidin-1- yl)nicotinoyl)piperidin- 4-yl)butyl)-3-(pyridin- 3-yl)acrylamide |
| | 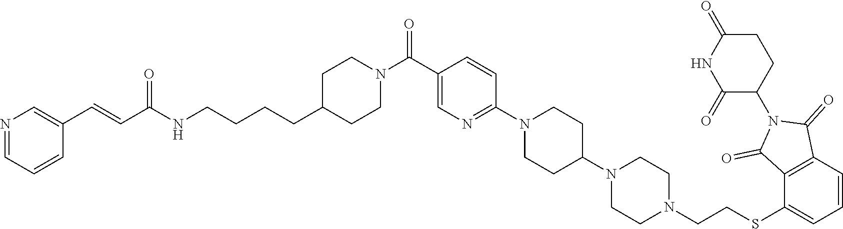 [SMILES: O=C(/C=C/c1cccnc1)NCCCCC1CCN(C(=O)c2ccc(N3CCC(N4CCN(CCSc5cccc6c5C(=O)N(C5CCC(=O)NC5=O)C6=O)CC4)CC3)nc2)CC1] | (E)-N-(4-(1-(6-(4-(4-(2- ((2-(2,6-dioxopiperidin- 3-yl)-1,3- dioxoisoindolin-4- yl)thio)ethyl)piperazin- 1-yl)piperidin-1- yl)nicotinoyl)piperidin- 4-yl)butyl)-3-(pyridin- 3-yl)acrylamide | (E)-N-(4-(1-(6-(4-(4- (2-((2-(2,6- dioxopiperidin-3-yl)- 1,3-dioxoisoindolin- 4-yl)thio)ethyl) piperazin-1- yl)piperidin-1- yl)nicotinoyl)piperidin- 4-yl)butyl)-3-(pyridin- 3-yl)acrylamide |
| | 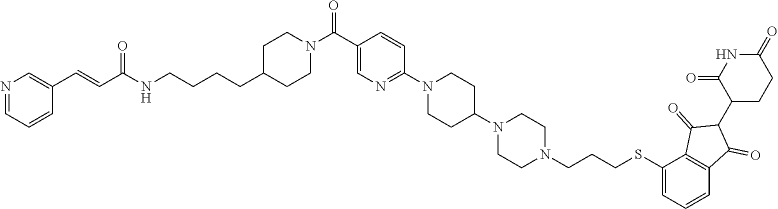 [SMILES: O=C(/C=C/c1cccnc1)NCCCCC1CCN(C(=O)c2ccc(N3CCC(N4CCN(CCCSc5cccc6c5C(=O)C(C5CCC(=O)NC5=O)C6=O)CC4)CC3)nc2)CC1] | (E)-N-(4-(1-(6-(4-(4-(3- ((2-(2,6-dioxopiperidin- 3-yl)-1,3- dioxoisoindolin-4- yl)thio)propyl) piperazin-1- yl)piperidin-1- yl)nicotinoyl)piperidin- 4-yl)butyl)-3-(pyridin- 3-yl)acrylamide | (E)-N-(4-(1-(6-(4-(4- (3-((2-(2,6- dioxopiperidin-3-yl)- 1,3-dioxoisoindolin- 4-yl)thio)propyl) piperazin-1- yl)piperidin-1- yl)nicotinoyl) piperidin-4-yl)butyl)- 3-(pyridin- 3-yl)acrylamide |
| | 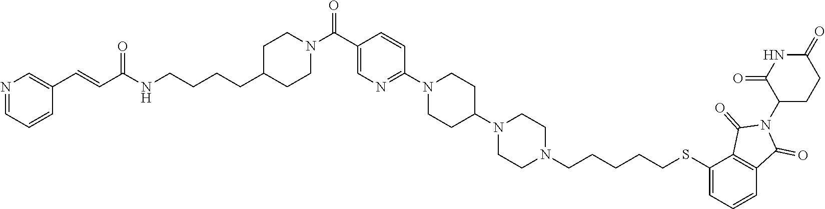 [SMILES: O=C(/C=C/c1cccnc1)NCCCCC1CCN(C(=O)c2ccc(N3CCC(N4CCN(CCCCCSc5cccc6c5C(=O)N(C5CCC(=O)NC5=O)C6=O)CC4)CC3)nc2)CC1] | (E)-N-(4-(1-(6-(4-(4-(5- ((2-(2,6-dioxopiperidin- 3-yl)-1,3- dioxoisoindolin-4- yl)thio)pentyl)piperazin- 1-yl)piperidin-1- yl)nicotinoyl)piperidin- 4-yl)butyl)-3-(pyridin- 3-yl)acrylamide | (E)-N-(4-(1-(6-(4-(4- (5-((2-(2,6- dioxopiperidin-3-yl)- 1,3-dioxoisoindolin- 4-yl)thio)pentyl) piperazin-1- yl)piperidin-1- yl)nicotinoyl) piperidin-4- yl)butyl)-3-(pyridin- 3-yl)acrylamide |
| | 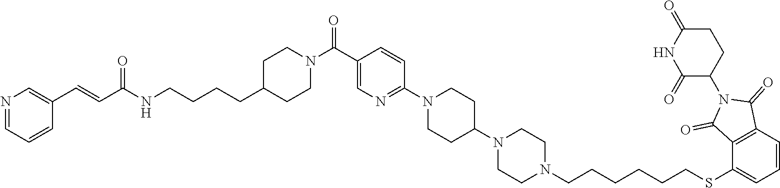 [SMILES: O=C(/C=C/c1cccnc1)NCCCCC1CCN(C(=O)c2ccc(N3CCC(N4CCN(CCCCCCSc5cccc6c5C(=O)N(C5CCC(=O)NC5=O)C6=O)CC4)CC3)nc2)CC1] | (E)-N-(4-(1-(6-(4-(4-(6- ((2-(2,6-dioxopiperidin- 3-yl)-1,3- dioxoisoindolin-4- yl)thio)hexyl)piperazin- 1-yl)piperidin-1- yl)nicotinoyl)piperidin- 4-yl)butyl)-3-(pyridin- 3-yl)acrylamide | (E)-N-(4-(1-(6-(4-(4- (6-((2-(2,6- dioxopiperidin-3-yl)- 1,3-dioxoisoindolin- 4-yl)thio)hexyl) piperazin-1- yl)piperidin-1- yl)nicotinoyl) piperidin-4- yl)butyl)-3-(pyridin- 3-yl)acrylamide |
| | 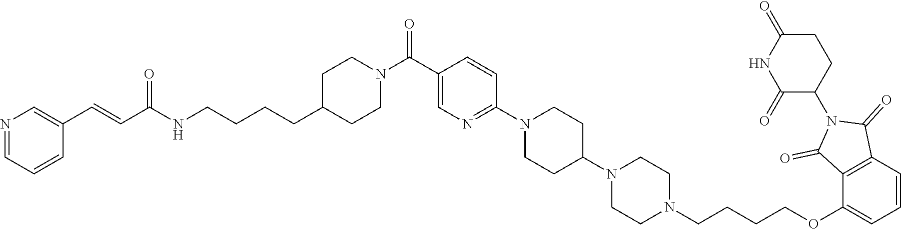 [SMILES: O=C(/C=C/c1cccnc1)NCCCCC1CCN(C(=O)c2ccc(N3CCC(N4CCN(CCCCOc5cccc6c5C(=O)N(C5CCC(=O)NC5=O)C6=O)CC4)CC3)nc2)CC1] | (E)-N-(4-(1-(6-(4-(4-(4- ((2-(2,6-dioxopiperidin- 3-yl)-1,3- dioxoisoindolin-4- yl)oxy)butyl)piperazin- 1-yl)piperidin-1- yl)nicotinoyl)piperidin- 4-yl)butyl)-3-(pyridin- 3-yl)acrylamide | (E)-N-(4-(1-(6-(4-(4- (4-((2-(2,6- dioxopiperidin-3-yl)- 1,3-dioxoisoindolin- 4-yl)oxy)butyl) piperazin-1- yl)piperidin-1- yl)nicotinoyl) piperidin-4- yl)butyl)-3-(pyridin- 3-yl)acrylamide |
| | 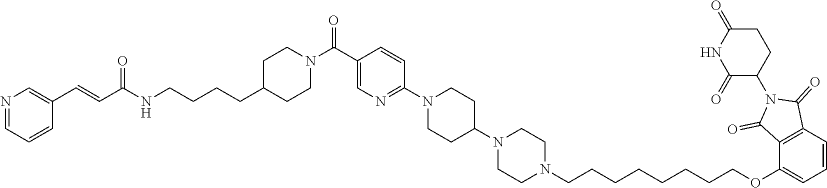 [SMILES: O=C(/C=C/c1cccnc1)NCCCCC1CCN(C(=O)c2ccc(N3CCC(N4CCN(CCCCCCCCOc5cccc6c5C(=O)N(C5CCC(=O)NC5=O)C6=O)CC4)CC3)nc2)CC1] | (E)-N-(4-(1-(6-(4-(4-(8- ((2-(2,6-dioxopiperidin- 3-yl)-1,3- dioxoisoindolin-4- yl)oxy)octyl)piperazin- 1-yl)piperidin-1- yl)nicotinoyl)piperidin- 4-yl)butyl)-3-(pyridin- 3-yl)acrylamide | (E)-N-(4-(1-(6-(4-(4- (8-((2-(2,6- dioxopiperidin-3-yl)- 1,3-dioxoisoindolin- 4-yl)oxy)octyl) piperazin-1- yl)piperidin-1- yl)nicotinoyl) piperidin-4- yl)butyl)-3-(pyridin- 3-yl)acrylamide |
| | 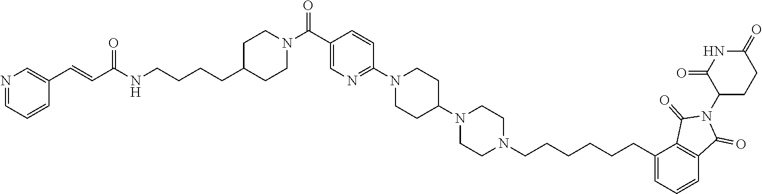 [SMILES: O=C(/C=C/c1cccnc1)NCCCCC1CCN(C(=O)c2ccc(N3CCC(N4CCN(CCCCCCc5cccc6c5C(=O)N(C5CCC(=O)NC5=O)C6=O)CC4)CC3)nc2)CC1] | (E)-N-(4-(1-(6-(4-(4-(6- (2-(2,6-dioxopiperidin- 3-yl)-1,3- dioxoisoindolin-4- yl)hexyl)piperazin-1- yl)piperidin-1- yl)nicotinoyl)piperidin- 4-yl)butyl)-3-(pyridin- 3-yl)acrylamide | (E)-N-(4-(1-(6-(4-(4- (6-(2-(2,6- dioxopiperidin-3-yl)- 1,3-dioxoisoindolin- 4-yl)hexyl)piperazin- 1-yl)piperidin-1- yl)nicotinoyl) piperidin4- yl)butyl)-3-(pyridin- 3-yl)acrylamide |
| | 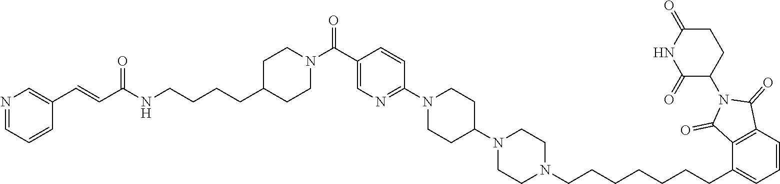 [SMILES: O=C(/C=C/c1cccnc1)NCCCCC1CCN(C(=O)c2ccc(N3CCC(N4CCN(CCCCCCCc5cccc6c5C(=O)N(C5CCC(=O)NC5=O)C6=O)CC4)CC3)nc2)CC1] | (E)-N-(4-(1-(6-(4-(4-(7- (2-(2,6-dioxopiperidin- 3-yl)-1,3- dioxoisoindolin-4- yl)heptyl)piperazin- 1-yl)piperidin-1- yl)nicotinoyl)piperidin- 4-yl)butyl)-3-(pyridin- 3-yl)acrylamide | (E)-N-(4-(1-(6-(4-(4- (7-(2-(2,6- dioxopiperidin-3-yl)- 1,3-dioxoisoindolin- 4-yl)heptyl)piperazin- 1-yl)piperidin-1- yl)nicotinoyl)piperidin- 4-yl)butyl)-3-(pyridin- 3-yl)acrylamide |
| | 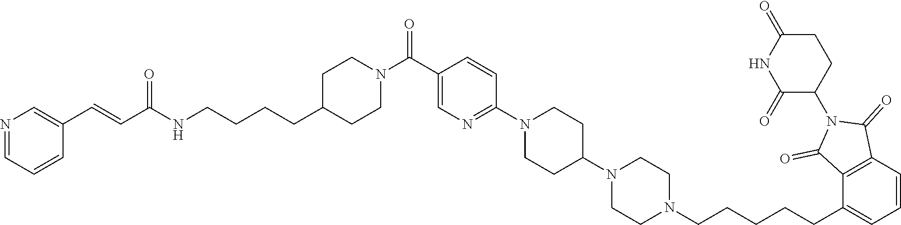 [SMILES: O=C(/C=C/c1cccnc1)NCCCCC1CCN(C(=O)c2ccc(N3CCC(N4CCN(CCCCCc5cccc6c5C(=O)N(C5CCC(=O)NC5=O)C6=O)CC4)CC3)nc2)CC1] | (E)-N-(4-(1-(6-(4-(4-(5- (2-(2,6-dioxopiperidin- 3-yl)-1,3- dioxoisoindolin-4- yl)pentyl)piperazin- 1-yl)piperidin-1- yl)nicotinoyl)piperidin- 4-yl)butyl)-3-(pyridin- 3-yl)acrylamide | (E)-N-(4-(1-(6-(4-(4- (5-(2-(2,6- dioxopiperidin-3-yl)- 1,3-dioxoisoindolin- 4-yl)pentyl)piperazin- 1-yl)piperidin-1- yl)nicotinoyl)piperidin- 4-yl)butyl)-3-(pyridin- 3-yl)acrylamide |
| | 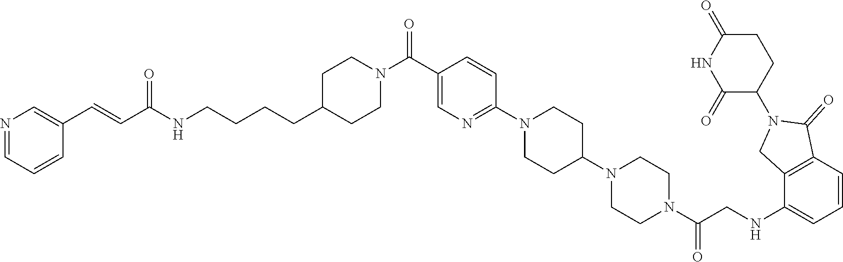 [SMILES: O=C(/C=C/c1cccnc1)NCCCCC1CCN(C(=O)c2ccc(N3CCC(N4CCN(C(=O)CNc5cccc6c5CN(C5CCC(=O)NC5=O)C6=O)CC4)CC3)nc2)CC1] | (E)-N-(4-(1-(6-(4-(4- ((2-(2,6-dioxopiperidin- 3-yl)-1-oxoisoindolin- 4-yl)glycyl)piperazin- 1-yl)piperidin-1- yl)nicotinoyl)piperidin- 4-yl)butyl)-3-(pyridin- 3-yl)acrylamide | (E)-N-(4-(1-(6-(4-(4- ((2-(2,6- dioxopiperidin-3-yl)- 1-oxoisoindolin-4- yl)glycyl)piperazin- 1-yl)piperidin-1- yl)nicotinoyl)piperidin- 4-yl)butyl)-3-(pyridin- 3-yl)acrylamide |
| | 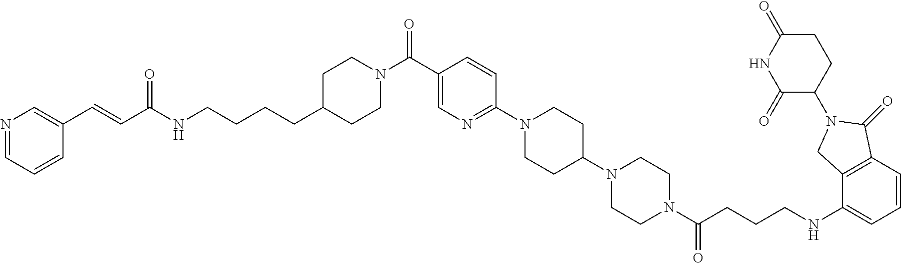 [SMILES: O=C(/C=C/c1cccnc1)NCCCCC1CCN(C(=O)c2ccc(N3CCC(N4CCN(C(=O)CCCNc5cccc6c5CN(C5CCC(=O)NC5=O)C6=O)CC4)CC3)nc2)CC1] | (E)-N-(4-(1-(6-(4-(4-(4- ((2-(2,6-dioxopiperidin- 3-yl)-1-oxoisoindolin- 4-yl)amino)butanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (4-((2-(2,6- dioxopiperidin-3-yl)- 1-oxoisoindolin-4- yl)amino)butanoyl) piperazin-1- yl)piperidin-1- yl)nicotinoyl)piperidin- 4-yl)butyl)-3-(pyridin- 3-yl)acrylamide |
| | 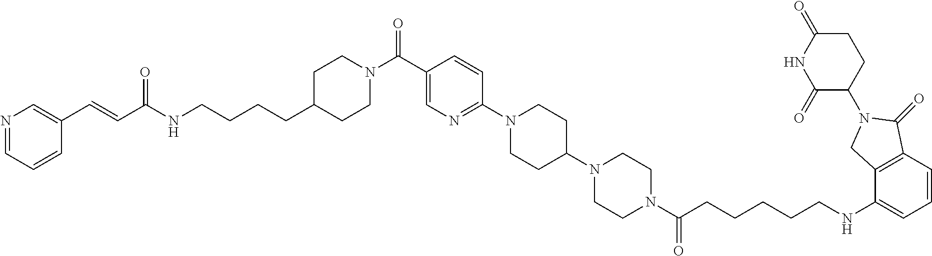 [SMILES: O=C(/C=C/c1cccnc1)NCCCCC1CCN(C(=O)c2ccc(N3CCC(N4CCN(C(=O)CCCCCNc5cccc6c5CN(C5CCC(=O)NC5=O)C6=O)CC4)CC3)nc2)CC1] | (E)-N-(4-(1-(6-(4-(4-(6- ((2-(2,6-dioxopiperidin- 3-yl)-1-oxoisoindolin- 4-yl)amino)hexanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (6-((2-(2,6- dioxopiperidin-3-yl)- 1-oxoisoindolin-4- yl)amino)hexanoyl) piperazin-1- yl)piperidin-1- yl)nicotinoyl) piperidin-4- yl)butyl)-3-(pyridin- 3-yl)acrylamide |
| | 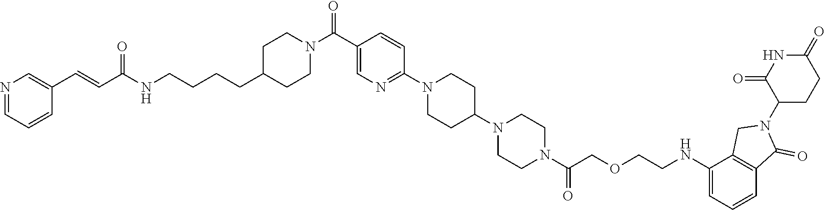 [SMILES: O=C(/C=C/c1cccnc1)NCCCCC1CCN(C(=O)c2ccc(N3CCC(N4CCN(C(=O)COCCNc5cccc6c5CN(C5CCC(=O)NC5=O)C6=O)CC4)CC3)nc2)CC1] | (E)-N-(4-(1-(6-(4-(4-(2- (2-((2-(2,6- dioxopiperidin-3-yl)- 1-oxoisoindolin-4- yl)amino)ethoxy) acetyl)piperazin-1- yl)piperidin-1- yl)nicotinoyl)piperidin- 4-yl)butyl)-3-(pyridin- 3-yl)acrylamide | (E)-N-(4-(1-(6-(4-(4- (2-(2-((2-(2,6- dioxopiperidin-3-yl)- 1-oxoisoindolin-4- yl)amino)ethoxy) acetyl)piperazin-1- yl)piperidin-1- yl)nicotinoyl) piperidin-4-yl) butyl)-3-(pyridin- 3-yl)acrylamide |
| | 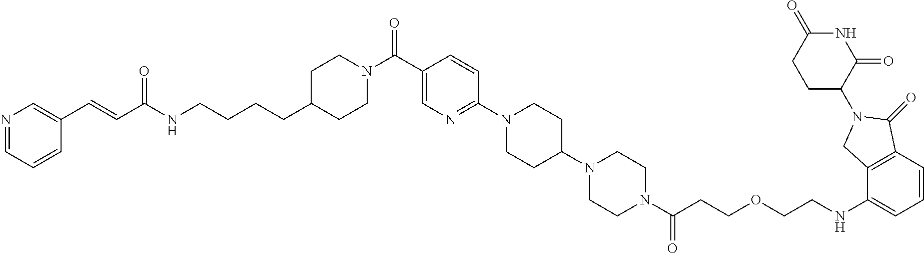 [SMILES: O=C(/C=C/c1cccnc1)NCCCCC1CCN(C(=O)c2ccc(N3CCC(N4CCN(C(=O)CCOCCNc5cccc6c5CN(C5CCC(=O)NC5=O)C6=O)CC4)CC3)nc2)CC1] | (E)-N-(4-(1-(6-(4-(4-(3- (2-((2-(2,6- dioxopiperidin-3-yl)- 1-oxoisoindolin-4- yl)amino)ethoxy) propanoyl)piperazin- 1-yl)piperidin-1- yl)nicotinoyl)piperidin- 4-yl)butyl)-3-(pyridin- 3-yl)acrylamide | (E)-N-(4-(1-(6-(4-(4- (3-(2-((2-(2,6- dioxopiperidin-3-yl)- 1-oxoisoindolin-4- yl)amino)ethoxy) propanoyl)piperazin- 1-yl)piperidin-1- yl)nicotinoyl) piperidin-4- yl)butyl)-3-(pyridin- 3-yl)acrylamide |
| | 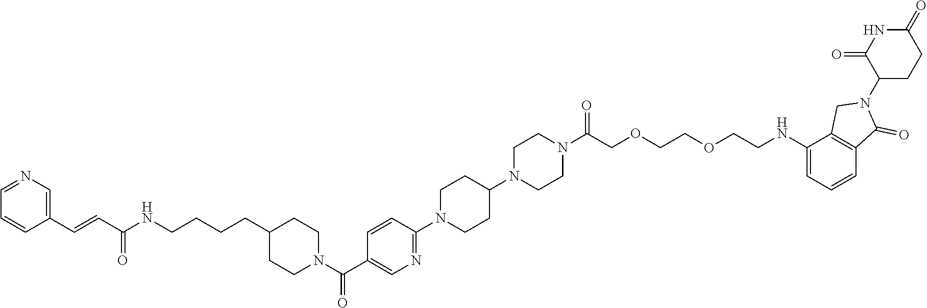 [SMILES: O=C(/C=C/c1cccnc1)NCCCCC1CCN(C(=O)c2ccc(N3CCC(N4CCN(C(=O)COCCOCCNc5cccc6c5CN(C5CCC(=O)NC5=O)C6=O)CC4)CC3)nc2)CC1] | (E)-N-(4-(1-(6-(4-(4-(2- (2-(2-((2-(2,6- dioxopiperidin-3-yl)- 1-oxoisoindolin-4- yl)amino)ethoxy) ethoxy)acetyl) piperazin-1- yl)piperidin-1- yl)nicotinoyl) piperidin-4- yl)butyl)-3-(pyridin- 3-yl)acrylamide | (E)-N-(4-(1-(6-(4-(4- (2-(2-(2-((2-(2,6- dioxopiperidin-3-yl)- 1-oxoisoindolin-4- yl)amino)ethoxy) ethoxy)acetyl) piperazin-1- yl)piperidin-1- yl)nicotinoyl) piperidin-4- yl)butyl)-3-(pyridin- 3-yl)acrylamide |
| | 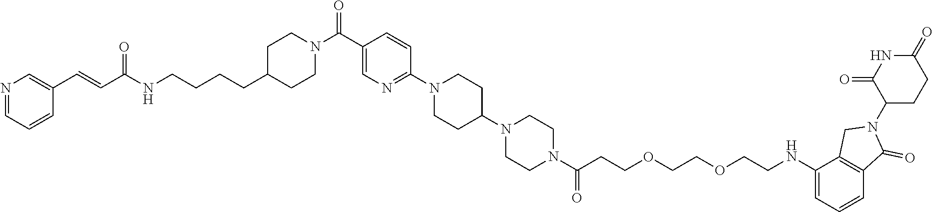 [SMILES: O=C(/C=C/c1cccnc1)NCCCCC1CCN(C(=O)c2ccc(N3CCC(N4CCN(C(=O)CCOCCOCCNc5cccc6c5CN(C5CCC(=O)NC5=O)C6=O)CC4)CC3)nc2)CC1] | (E)-N-(4-(1-(6-(4-(4-(3- (2-(2-((2-(2,6- dioxopiperidin-3-yl)- 1-oxoisoindolin-4- yl)amino)ethoxy) ethoxy)propanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (3-(2-(2-((2-(2,6- dioxopiperidin-3-yl)- 1-oxoisoindolin-4- yl)amino)ethoxy) ethoxy)propanoyl) piperazin-1- yl)piperidin-1- yl)nicotinoyl) piperidin-4- yl)butyl)-3-(pyridin- 3-yl)acrylamide |
| | 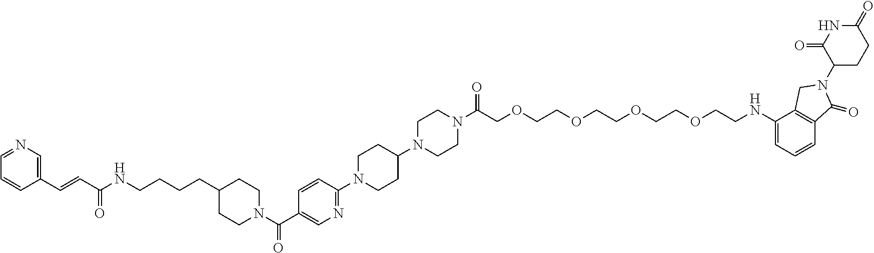 [SMILES: O=C(/C=C/c1cccnc1)NCCCCC1CCN(C(=O)c2ccc(N3CCC(N4CCN(C(=O)COCCOCCOCCOCCNc5cccc6c5CN(C5CCC(=O)NC5=O)C6=O)CC4)CC3)nc2)CC1] | (E)-N-(4-(1-(6-(4-(4- (14-((2-(2,6- dioxopiperidin-3-yl)- 1-oxoisoindolin-4- yl)amino)-3,6,9,12- tetraoxatetradecanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (14-((2-(2,6- dioxopiperidin-3-yl)- 1-oxoisoindolin-4- yl)amino)-3,6,9,12- tetraoxatetradecanoyl) piperazin-1- yl)piperidin-1- yl)nicotinoyl)piperidin- 4-yl)butyl)-3-(pyridin- 3-yl)acrylamide |
| | 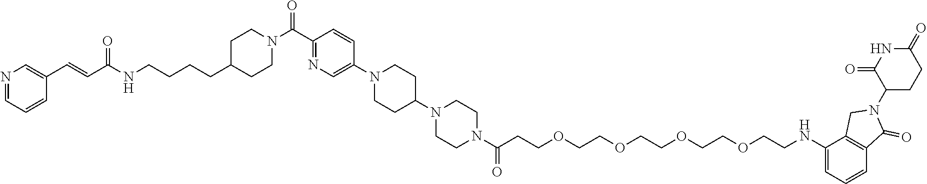 [SMILES: O=C(/C=C/c1cccnc1)NCCCCC1CCN(C(=O)c2ccc(N3CCC(N4CCN(C(=O)CCOCCOCCOCCOCCNc5cccc6c5CN(C5CCC(=O)NC5=O)C6=O)CC4)CC3)cn2)CC1] | (E)-N-(4-(1-(6-(4-(4-(1- ((2-(2,6-dioxopiperidin- 3-yl)-1-oxoisoindolin- 4-yl)amino)-3,6,9,12- tetraoxapentadecan- 15-oyl)piperazin-1- yl)piperidin-1- yl)nicotinoyl)piperidin- 4-yl)butyl)-3-(pyridin- 3-yl)acrylamide | (E)-N-(4-(1-(6-(4-(4- (1-((2-(2,6- dioxopiperidin-3-yl)- 1-oxoisoindolin-4- yl)amino)-3,6,9,12- tetraoxapentadecan- 15-oyl)piperazin-1- yl)piperidin-1- yl)nicotinoyl) piperidin-4- yl)butyl)-3-(pyridin- 3-yl)acrylamide |
| | 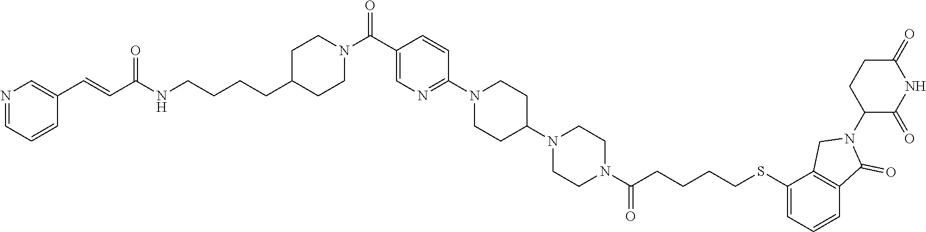 [SMILES: O=C(/C=C/c1cccnc1)NCCCCC1CCN(C(=O)c2ccc(N3CCC(N4CCN(C(=O)CCCCSc5cccc6c5CN(C5CCC(=O)NC5=O)C6=O)CC4)CC3)nc2)CC1] | (E)-N-(4-(1-(6-(4-(4-(5- ((2-(2,6-dioxopiperidin- 3-yl)-1-oxoisoindolin- 4-yl)thio)pentanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (5-((2-(2,6- dioxopiperidin-3-yl)- 1-oxoisoindolin-4- yl)thio)pentanoyl) piperazin-1- yl)piperidin-1- yl)nicotinoyl) piperidin-4- yl)butyl)-3-(pyridin- 3-yl)acrylamide |
| | 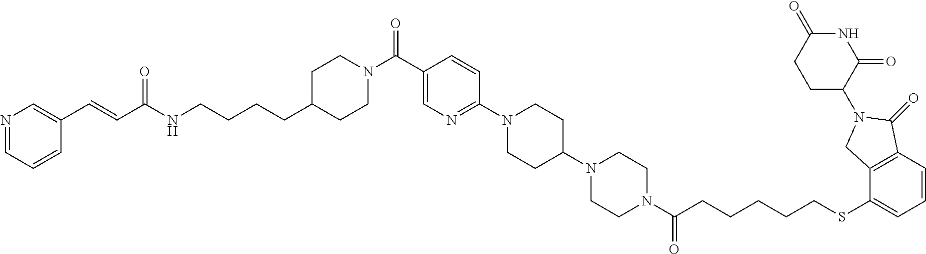 [SMILES: O=C(/C=C/c1cccnc1)NCCCCC1CCN(C(=O)c2ccc(N3CCC(N4CCN(C(=O)CCCCCSc5cccc6c5CN(C5CCC(=O)NC5=O)C6=O)CC4)CC3)nc2)CC1] | (E)-N-(4-(1-(6-(4-(4-(6- ((2-(2,6-dioxopiperidin- 3-yl)-1-oxoisoindolin- 4-yl)thio)hexanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (6-((2-(2,6- dioxopiperidin-3-yl)- 1-oxoisoindolin-4- yl)thio)hexanoyl) piperazin-1- yl)piperidin-1- yl)nicotinoyl) piperidin-4- yl)butyl)-3-(pyridin- 3-yl)acrylamide |
| | 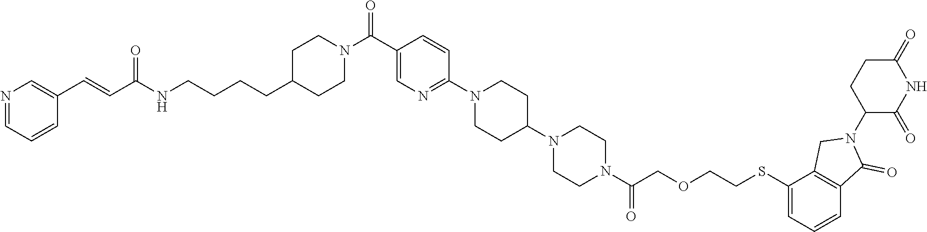 [SMILES: O=C(/C=C/c1cccnc1)NCCCCC1CCN(C(=O)c2ccc(N3CCC(N4CCN(C(=O)COCCSc5cccc6c5CN(C5CCC(=O)NC5=O)C6=O)CC4)CC3)nc2)CC1] | (E)-N-(4-(1-(6-(4-(4-(2- (2-((2-(2,6- dioxopiperidin-3-yl)- 1-oxoisoindolin-4- yl)thio)ethoxy)acetyl) piperazin-1- yl)piperidin-1- yl)nicotinoyl)piperidin- 4-yl)butyl)-3-(pyridin- 3-yl)acrylamide | (E)-N-(4-(1-(6-(4-(4- (2-(2-((2-(2,6- dioxopiperidin-3-yl)- 1-oxoisoindolin-4- yl)thio)ethoxy)acetyl) piperazin-1-yl) piperidin-1- yl)nicotinoyl) piperidin-4-yl) butyl)-3-(pyridin- 3-yl)acrylamide |
| | 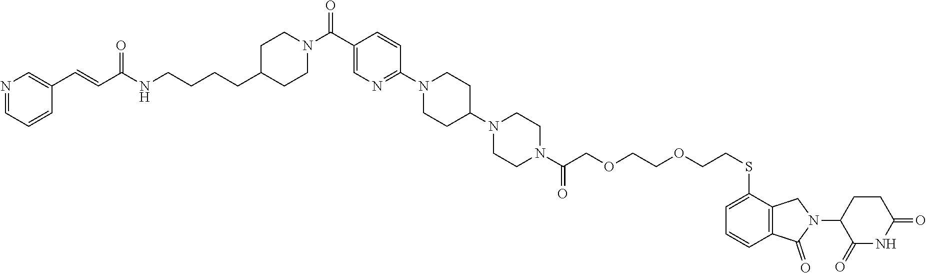 [SMILES: O=C(/C=C/c1cccnc1)NCCCCC1CCN(C(=O)c2ccc(N3CCC(N4CCN(C(=O)COCCOCCSc5cccc6c5CN(C5CCC(=O)NC5=O)C6=O)CC4)CC3)nc2)CC1] | (E)-N-(4-(1-(6-(4-(4-(2- (2-(2-((2-(2,6- dioxopiperidin-3-yl)- 1-oxoisoindolin-4- yl)thio)ethoxy)ethoxy) acetyl)piperazin-1- yl)piperidin-1- yl)nicotinoyl)piperidin- 4-yl)butyl)-3-(pyridin- 3-yl)acrylamide | (E)-N-(4-(1-(6-(4-(4- (2-(2-(2-((2-(2,6- dioxopiperidin-3- yl)-1-oxoisoindolin- 4-yl)thio)ethoxy) ethoxy)acetyl) piperazin-1- yl)piperidin-1- yl)nicotinoyl) piperidin-4- yl)butyl)-3-(pyridin- 3-yl)acrylamide |
| | 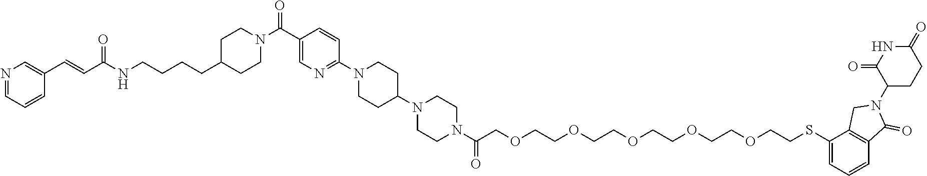 [SMILES: O=C(/C=C/c1cccnc1)NCCCCC1CCN(C(=O)c2ccc(N3CCC(N4CCN(C(=O)COCCOCCOCCOCCOCCSc5cccc6c5CN(C5CCC(=O)NC5=O)C6=O)CC4)CC3)nc2)CC1] | (E)-N-(4-(1-(6-(4-(4- (17-((2-(2,6- dioxopiperidin-3-yl)- 1-oxoisoindolin-4- yl)thio)-3,6,9,12,15- pentaoxaheptadecanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (17-((2-(2,6- dioxopiperidin-3-yl)- 1-oxoisoindolin-4- yl)thio)-3,6,9,12,15- pentaoxaheptadecanoyl) piperazin-1- yl)piperidin-1- yl)nicotinoyl)piperidin- 4-yl)butyl)-3-(pyridin- 3-yl)acrylamide |
| | 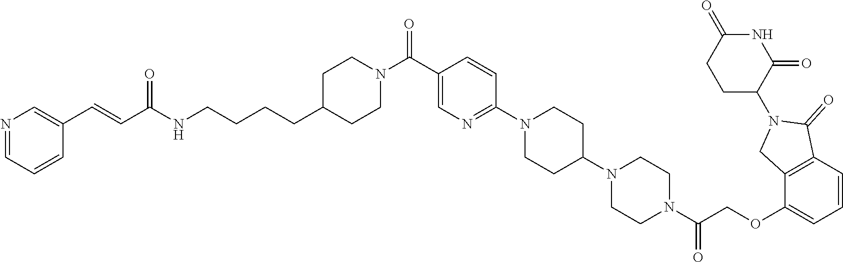 [SMILES: O=C(/C=C/c1cccnc1)NCCCCC1CCN(C(=O)c2ccc(N3CCC(N4CCN(C(=O)COc5cccc6c5CN(C5CCC(=O)NC5=O)C6=O)CC4)CC3)nc2)CC1] | (E)-N-(4-(1-(6-(4-(4-(2- ((2-(2,6-dioxopiperidin- 3-yl)-1-oxoisoindolin- 4-yl)oxy)acetyl) piperazin-1- yl)piperidin-1- yl)nicotinoyl)piperidin- 4-yl)butyl)-3-(pyridin- 3-yl)acrylamide | (E)-N-(4-(1-(6-(4-(4- (2-((2-(2,6- dioxopiperidin-3-yl)- 1-oxoisoindolin-4- yl)oxy)acetyl) piperazin-1- yl)piperidin-1- yl)nicotinoyl)piperidin- 4-yl)butyl)-3-(pyridin- 3-yl)acrylamide |
| | 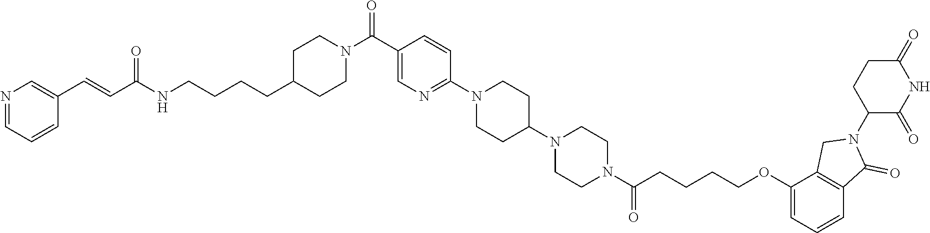 [SMILES: O=C(/C=C/c1cccnc1)NCCCCC1CCN(C(=O)c2ccc(N3CCC(N4CCN(C(=O)CCCCOc5cccc6c5CN(C5CCC(=O)NC5=O)C6=O)CC4)CC3)nc2)CC1] | (E)-N-(4-(1-(6-(4-(4-(5- ((2-(2,6-dioxopiperidin- 3-yl)-1-oxoisoindolin- 4-yl)oxy)pentanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (5-((2-(2,6- dioxopiperidin-3-yl)- 1-oxoisoindolin-4- yl)oxy)pentanoyl) piperazin-1- yl)piperidin-1- yl)nicotinoyl)piperidin- 4-yl)butyl)-3-(pyridin- 3-yl)acrylamide |
| | 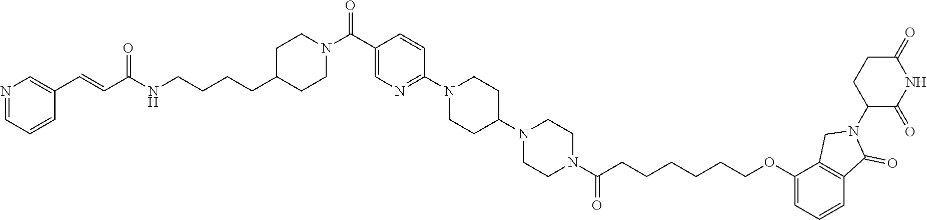 [SMILES: O=C(/C=C/c1cccnc1)NCCCCC1CCN(C(=O)c2ccc(N3CCC(N4CCN(C(=O)CCCCCCOc5cccc6c5CN(C5CCC(=O)NC5=O)C6=O)CC4)CC3)nc2)CC1] | (E)-N-(4-(1-(6-(4-(4-(7- ((2-(2,6-dioxopiperidin- 3-yl)-1-oxoisoindolin- 4-yl)oxy)heptanoyl) piperazin-1- yl)piperidin-1- yl)nicotinoyl)piperidin- 4-yl)butyl)-3-(pyridin- 3-yl)acrylamide | (E)-N-(4-(1-(6-(4-(4- (7-((2-(2,6- dioxopiperidin-3-yl)- 1-oxoisoindolin-4- yl)oxy)heptanoyl) piperazin-1- yl)piperidin-1- yl)nicotinoyl)piperidin- 4-yl)butyl)-3-(pyridin- 3-yl)acrylamide |
| | 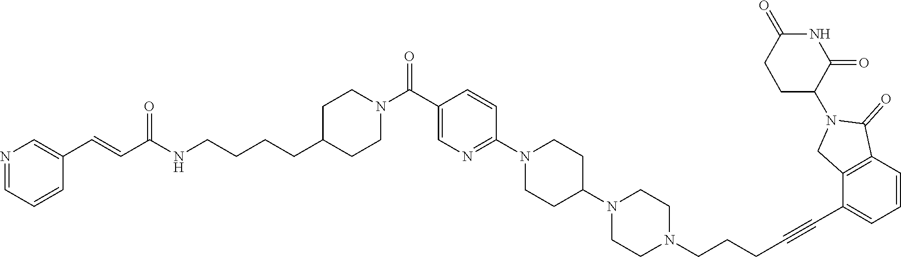 [SMILES: O=C(/C=C/c1cccnc1)NCCCCC1CCN(C(=O)c2ccc(N3CCC(N4CCN(CCCC#Cc5cccc6c5CN(C5CCC(=O)NC5=O)C6=O)CC4)CC3)nc2)CC1] | (E)-N-(4-(1-(6-(4-(4-(5- (2-(2,6-dioxopiperidin- 3-yl)-1-oxoisoindolin- 4-yl)pent-4-yn-1- yl)piperazin-1- yl)piperidin-1- yl)nicotinoyl)piperidin- 4-yl)butyl)-3-(pyridin- 3-yl)acrylamide | (E)-N-(4-(1-(6-(4-(4- (5-(2-(2,6- dioxopiperidin-3-yl)- 1-oxoisoindolin-4- yl)pent-4-yn-1- yl)piperazin-1- yl)piperidin-1- yl)nicotinoyl) piperidin-4- yl)butyl)-3-(pyridin- 3-yl)acrylamide |
| | 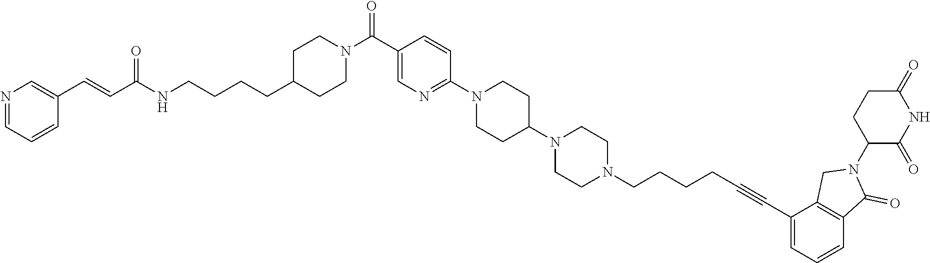 [SMILES: O=C(/C=C/c1cccnc1)NCCCCC1CCN(C(=O)c2ccc(N3CCC(N4CCN(CCCCC#Cc5cccc6c5CN(C5CCC(=O)NC5=O)C6=O)CC4)CC3)nc2)CC1] | (E)-N-(4-(1-(6-(4-(4-(6- (2-(2,6-dioxopiperidin- 3-yl)-1-oxoisoindolin- 4-yl)hex-5-yn-1- yl)piperazin-1- yl)piperidin-1- yl)nicotinoyl)piperidin- 4-yl)butyl)-3-(pyridin- 3-yl)acrylamide | (E)-N-(4-(1-(6-(4-(4- (6-(2-(2,6- dioxopiperidin-3-yl)- 1-oxoisoindolin-4-yl) hex-5-yn-1-yl) piperazin-1- yl)piperidin-1- yl)nicotinoyl)piperidin- 4-yl)butyl)-3-(pyridin- 3-yl)acrylamide |
| | 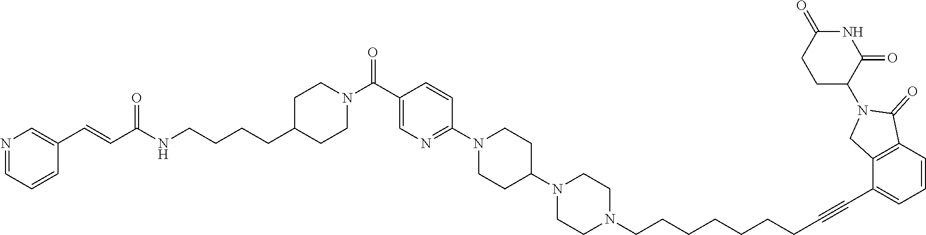 [SMILES: O=C(/C=C/c1cccnc1)NCCCCC1CCN(C(=O)c2ccc(N3CCC(N4CCN(CCCCCCCC#Cc5cccc6c5CN(C5CCC(=O)NC5=O)C6=O)CC4)CC3)nc2)CC1] | (E)-N-(4-(1-(6-(4-(4-(9- (2-(2,6-dioxopiperidin- 3-yl)-1-oxoisoindolin- 4-yl)non-8-yn-1- yl)piperazin-1- yl)piperidin-1- yl)nicotinoyl)piperidin- 4-yl)butyl)-3-(pyridin- 3-yl)acrylamide | (E)-N-(4-(1-(6-(4-(4- (9-(2-(2,6- dioxopiperidin-3-yl)- 1-oxoisoindolin-4- yl)non-8-yn-1- yl)piperazin-1- yl)piperidin-1 - yl)nicotinoyl) piperidin-4- yl)butyl)-3-(pyridin- 3-yl)acrylamide |
| | 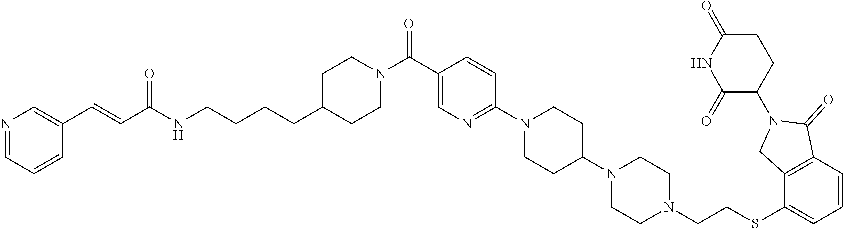 [SMILES: O=C(/C=C/c1cccnc1)NCCCCC1CCN(C(=O)c2ccc(N3CCC(N4CCN(CCSc5cccc6c5CN(C5CCC(=O)NC5=O)C6=O)CC4)CC3)nc2)CC1] | (E)-N-(4-(1-(6-(4-(4-(2- ((2-(2,6-dioxopiperidin- 3-yl)-1-oxoisoindolin- 4-yl)thio)ethyl) piperazin-1- yl)piperidin-1- yl)nicotinoyl)piperidin- 4-yl)butyl)-3-(pyridin- 3-yl)acrylamide | (E)-N-(4-(1-(6-(4-(4- (2-((2-(2,6- dioxopiperidin-3-yl)- 1-oxoisoindolin-4- yl)thio)ethyl) piperazin-1- yl)piperidin-1- yl)nicotinoyl) piperidin-4- yl)butyl)-3-(pyridin- 3-yl)acrylamide |
| | 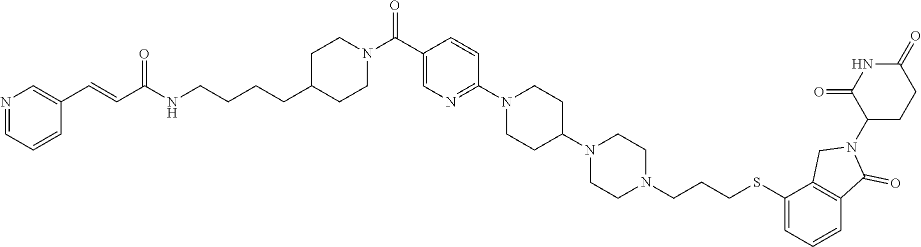 [SMILES: O=C(/C=C/c1cccnc1)NCCCCC1CCN(C(=O)c2ccc(N3CCC(N4CCN(CCCSc5cccc6c5CN(C5CCC(=O)NC5=O)C6=O)CC4)CC3)nc2)CC1] | (E)-N-(4-(1-(6-(4-(4-(3- ((2-(2,6-dioxopiperidin- 3-yl)-1-oxoisoindolin- 4-yl)thio)propyl) piperazin-1- yl)piperidin-1- yl)nicotinoyl) piperidin-4- yl)butyl)-3-(pyridin- 3-yl)acrylamide | (E)-N-(4-(1-(6-(4-(4- (3-((2-(2,6- dioxopiperidin-3-yl)- 1-oxoisoindolin-4- yl)thio)propyl) piperazi n-1-yl)piperidin-1- yl)nicotinoyl) piperidin- 4-yl)butyl)-3- (pyridin- 3-yl)acrylamide |
| | 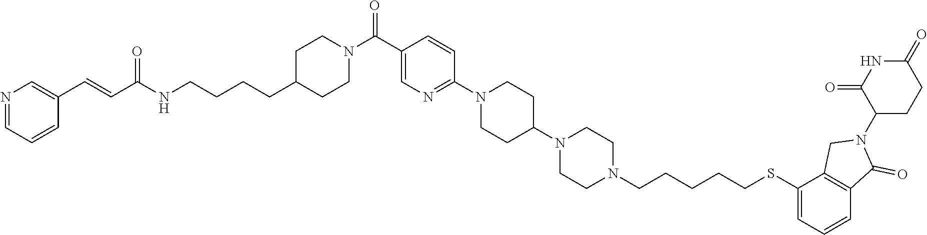 [SMILES: O=C(/C=C/c1cccnc1)NCCCCC1CCN(C(=O)c2ccc(N3CCC(N4CCN(CCCCCSc5cccc6c5CN(C5CCC(=O)NC5=O)C6=O)CC4)CC3)nc2)CC1] | (E)-N-(4-(1-(6-(4-(4-(5- ((2-(2,6-dioxopiperidin- 3-yl)-1-oxoisoindolin- 4-yl)thio)pentyl) piperazin-1- yl)piperidin-1- yl)nicotinoyl)piperidin- 4-yl)butyl)-3-(pyridin- 3-yl)acrylamide | (E)-N-(4-(1-(6-(4-(4- (5-((2-(2,6- dioxopiperidin-3-yl)- 1-oxoisoindolin-4- yl)thio)pentyl) piperazin-1- yl)piperidin-1- yl)nicotinoyl) piperidin-4- yl)butyl)-3-(pyridin- 3-yl)acrylamide |
| | 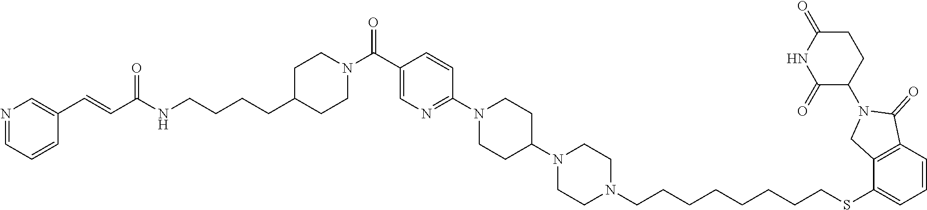 [SMILES: O=C(/C=C/c1cccnc1)NCCCCC1CCN(C(=O)c2ccc(N3CCC(N4CCN(CCCCCCCCSc5cccc6c5CN(C5CCC(=O)NC5=O)C6=O)CC4)CC3)nc2)CC1] | (E)-N-(4-(1-(6-(4-(4-(8- ((2-(2,6-dioxopiperidin- 3-yl)-1-oxoisoindolin- 4-yl)thio)octyl) piperazin-1- yl)piperidin-1- yl)nicotinoyl) piperidin-4- yl)butyl)-3-(pyridin- 3-yl)acrylamide | (E)-N-(4-(1-(6-(4-(4- (8-((2-(2,6- dioxopiperidin-3-yl)- 1-oxoisoindolin-4- yl)thio)octyl) piperazin-1- yl)piperidin-1- yl)nicotinoyl) piperidin-4- yl)butyl)-3-(pyridin- 3-yl)acrylamide |
| | 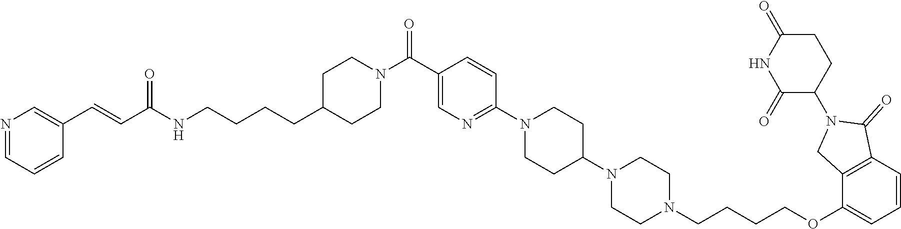 [SMILES: O=C(/C=C/c1cccnc1)NCCCCC1CCN(C(=O)c2ccc(N3CCC(N4CCN(CCCCOc5cccc6c5CN(C5CCC(=O)NC5=O)C6=O)CC4)CC3)nc2)CC1] | (E)-N-(4-(1-(6-(4-(4-(4- ((2-(2,6-dioxopiperidin- 3-yl)-1-oxoisoindolin- 4-yl)oxy)butyl) piperazin-1- yl)piperidin-1- yl)nicotinoyl) piperidin-4- yl)butyl)-3-(pyridin- 3-yl)acrylamide | (E)-N-(4-(1-(6-(4-(4- (4-((2-(2,6- dioxopiperidin-3-yl)- 1-oxoisoindolin-4- yl)oxy)butyl) piperazin-1- yl)piperidin-1- yl)nicotinoyl) piperidin-4- yl)butyl)-3-(pyridin- 3-yl)acrylamide |
| | 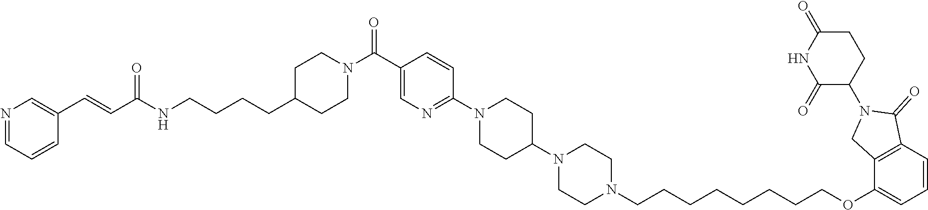 [SMILES: O=C(/C=C/c1cccnc1)NCCCCC1CCN(C(=O)c2ccc(N3CCC(N4CCN(CCCCCCCCOc5cccc6c5CN(C5CCC(=O)NC5=O)C6=O)CC4)CC3)nc2)CC1] | (E)-N-(4-(1-(6-(4-(4-(8- ((2-(2,6-dioxopiperidin- 3-yl)-1-oxoisoindolin- 4-yl)oxy)octyl) piperazin-1- yl)piperidin-1- yl)nicotinoyl) piperidin-4- yl)butyl)-3-(pyridin- 3-yl)acrylamide | (E)-N-(4-(1-(6-(4-(4- (8-((2-(2,6- dioxopiperidin-3-yl)- 1-oxoisoindolin-4- yl)oxy)octyl) piperazin-1- yl)piperidin-1- yl)nicotinoyl) piperidin-4- yl)butyl)-3-(pyridin- 3-yl)acrylamide |
| | 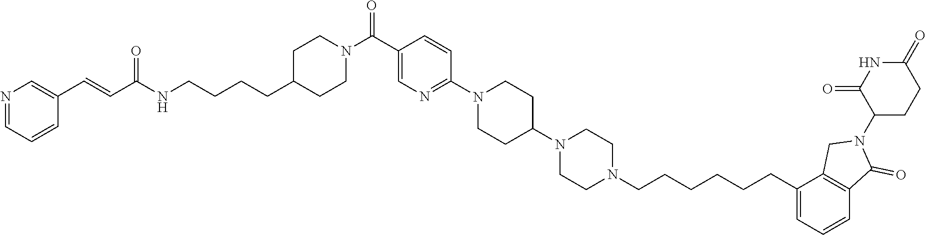 [SMILES: O=C(/C=C/c1cccnc1)NCCCCC1CCN(C(=O)c2ccc(N3CCC(N4CCN(CCCCCCc5cccc6c5CN(C5CCC(=O)NC5=O)C6=O)CC4)CC3)nc2)CC1] | (E)-N-(4-(1-(6-(4-(4-(6- (2-(2,6-dioxopiperidin- 3-yl)-1-oxoisoindolin- 4-yl)hexyl)piperazin- 1-yl)piperidin-1- yl)nicotinoyl)piperidin- 4-yl)butyl)-3-(pyridin- 3-yl)acrylamide | (E)-N-(4-(1-(6-(4-(4- (6-(2-(2,6- dioxopiperidin-3-yl)- 1-oxoisoindolin-4- yl)hexyl)piperazin- 1-yl)piperidin-1- yl)nicotinoyl) piperidin-4- yl)butyl)-3-(pyridin- 3-yl)acrylamide |
| | 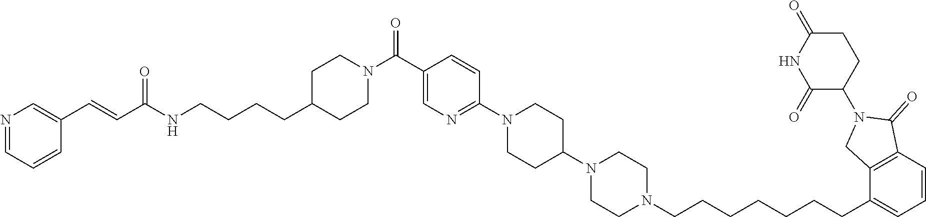 [SMILES: O=C(/C=C/c1cccnc1)NCCCCC1CCN(C(=O)c2ccc(N3CCC(N4CCN(CCCCCCCc5cccc6c5CN(C5CCC(=O)NC5=O)C6=O)CC4)CC3)nc2)CC1] | (E)-N-(4-(1-(6-(4-(4-(7- (2-(2,6-dioxopiperidin- 3-yl)-1-oxoisoindolin- 4-yl)heptyl)piperazin- 1-yl)piperidin-1- yl)nicotinoyl) piperidin-4- yl)butyl)-3-(pyridin- 3-yl)acrylamide | (E)-N-(4-(1-(6-(4-(4- (7-(2-(2,6- dioxopiperidin-3-yl)- 1-oxoisoindolin-4- yl)heptyl)piperazin- 1-yl)piperidin-1- yl)nicotinoyl) piperidin-4- yl)butyl)-3-(pyridin- 3-yl)acrylamide |
| | 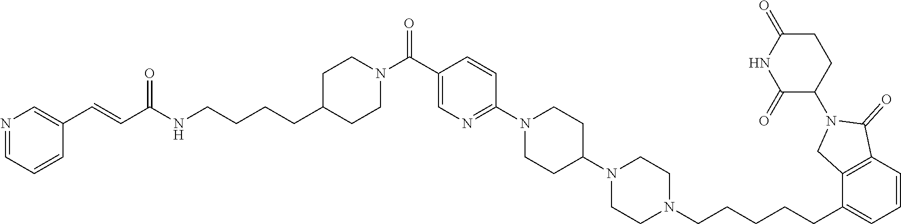 [SMILES: O=C(/C=C/c1cccnc1)NCCCCC1CCN(C(=O)c2ccc(N3CCC(N4CCN(CCCCCc5cccc6c5CN(C5CCC(=O)NC5=O)C6=O)CC4)CC3)nc2)CC1] | (E)-N-(4-(1-(6-(4-(4-(5- (2-(2,6-dioxopiperidin- 3-yl)-1-oxoisoindolin- 4-yl)pentyl)piperazin- 1-yl)piperidin-1- yl)nicotinoyl)piperidin- 4-yl)butyl)-3-(pyridin- 3-yl)acrylamide | (E)-N-(4-(1-(6-(4-(4- (5-(2-(2,6- dioxopiperidin-3-yl)- 1-oxoisoindolin-4- yl)pentyl)piperazin- 1-yl)piperidin-1- yl)nicotinoyl) piperidin-4- yl)butyl)-3-(pyridin- 3-yl)acrylamide |
| | 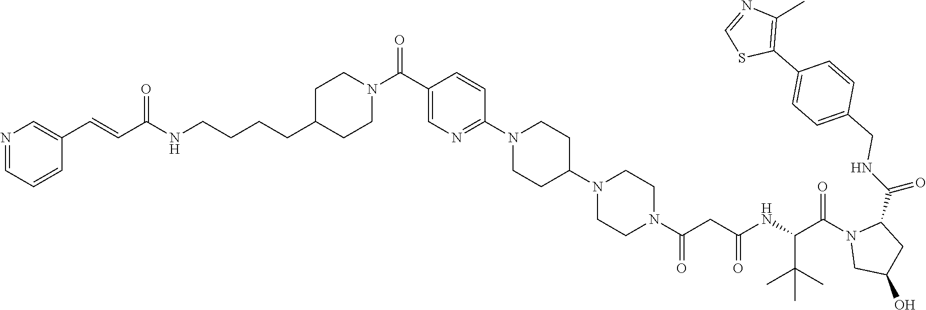 [SMILES: Cc1ncsc1-c1ccc(CNC(=O)[C@@H]2C[C@@H](O)CN2C(=O)[C@@H](NC(=O)CC(=O)N2CCN(C3CCN(c4ccc(C(=O)N5CCC(CCCCNC(=O)/C=C/c6cccnc6)CC5)cn4)CC3)CC2)C(C)(C)C)cc1] | (2S,4R)-1-((S)-3,3- dimethyl-2-(3-oxo-3- (4-(1-(5-(4-(4-((E)-3- (pyridin-3- yl)acrylamido)butyl) piperidine-1- carbonyl)pyridin-2- yl)piperidin-4- yl)piperazin-1- yl)propanamido) butanoyl)-4- hydroxy-N-(4-(4- methylthiazol-5- yl)benzyl)pyrrolidine- 2-carboxamide | (2S,4R)-1-((S)-3,3- dimethyl-2-(3-oxo-3- (4-(1-(5-(4-(4-((E)-3- (pyridin-3- yl)acrylamido)butyl) piperidine-1- carbonyl)pyridin-2- yl)piperidin-4- yl)piperazin-1- yl)propanamido) butanoyl)-4- hydroxy-N-(4-(4- methylthiazol-5- yl)benzyl)pyrrolidine- 2-carboxamide |
| | 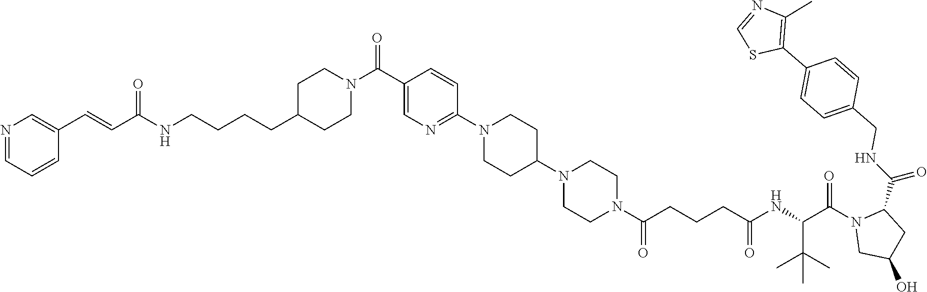 [SMILES: Cc1ncsc1-c1ccc(CNC(=O)[C@@H]2C[C@@H](O)CN2C(=O)[C@@H](NC(=O)CCCC(=O)N2CCN(C3CCN(c4ccc(C(=O)N5CCC(CCCCNC(=O)/C=C/c6cccnc6)CC5)cn4)CC3)CC2)C(C)(C)C)cc1] | (2S,4R)-1-((S)-3,3- dimethyl-2-(5-oxo- 5-(4-(1-(5-(4-(4- ((E)-3-(pyridin-3- yl)acrylamido)butyl) piperidine-1- carbonyl)pyridin-2- yl)piperidin-4- yl)piperazin-1- yl)pentanamido) butanoyl)-4- hydroxy-N-(4-(4- methylthiazol-5- yl)benzyl)pyrrolidine- 2-carboxamide | (2S,4R)-1-((S)-3,3- dimethyl-2-(5-oxo-5- (4-(1-(5-(4-(4-((E)-3- (pyridin-3- yl)acrylamido)butyl) piperidine-1- carbonyl)pyridin-2- yl)piperidin-4- yl)piperazin-1- yl)pentanamido) butanoyl)-4- hydroxy-N-(4-(4- methylthiazol-5- yl)benzyl)pyrrolidine- 2-carboxamide |
| | 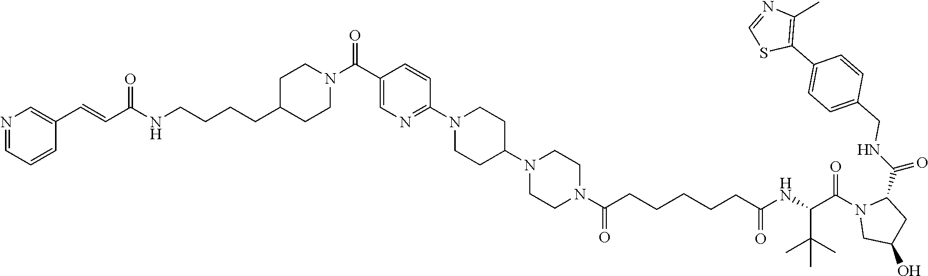 [SMILES: Cc1ncsc1-c1ccc(CNC(=O)[C@@H]2C[C@@H](O)CN2C(=O)[C@@H](NC(=O)CCCCCC(=O)N2CCN(C3CCN(c4ccc(C(=O)N5CCC(CCCCNC(=O)/C=C/c6cccnc6)CC5)cn4)CC3)CC2)C(C)(C)C)cc1] | (2S,4R)-1-((S)-3,3- dimethyl-2-(7-oxo-7- (4-(1-(5-(4-(4-((E)-3- (pyridin-3- yl)acrylamido)butyl) piperidine-1- carbonyl)pyridin-2- yl)piperidin-4- yl)piperazin-1- yl)heptanamido) butanoyl)-4- hydroxy-N-(4-(4- methylthiazol-5- yl)benzyl)pyrrolidine- 2-carboxamide | (2S,4R)-1-((S)-3,3- dimethyl-2-(7-oxo-7- (4-(1-(5-(4-(4-((E)-3- (pyridin-3- yl)acrylamido)butyl) piperidine-1- carbonyl)pyridin-2- yl)piperidin-4- yl)piperazin-1- yl)heptanamido) butanoyl)-4- hydroxy-N-(4-(4- methylthiazol-5- yl)benzyl)pyrrolidine- 2-carboxamide |
| | 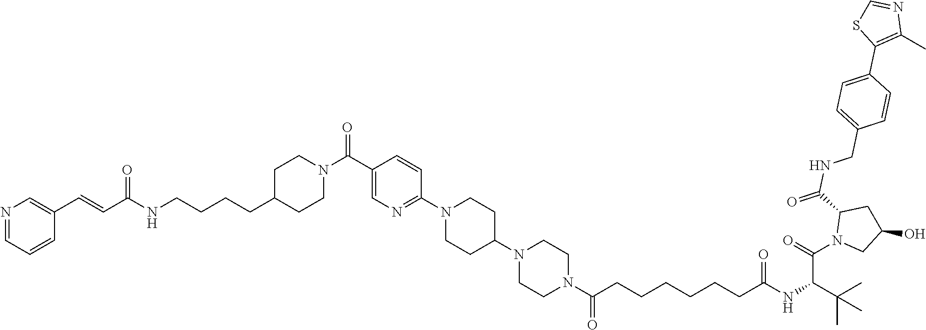 [SMILES: Cc1ncsc1-c1ccc(CNC(=O)[C@@H]2C[C@@H](O)CN2C(=O)[C@@H](NC(=O)CCCCCCC(=O)N2CCN(C3CCN(c4ccc(C(=O)N5CCC(CCCCNC(=O)/C=C/c6cccnc6)CC5)cn4)CC3)CC2)C(C)(C)C)cc1] | (2S,4R)-1-((S)-3,3- dimethyl-2-(8-oxo-8- (4-(1-(5-(4-(4-((E)-3- (pyridin-3- yl)acrylamido)butyl) piperidine-1- carbonyl)pyridin-2- yl)piperidin-4- yl)piperazin-1- yl)octanamido) butanoyl)-4- hydroxy-N-(4-(4- methylthiazol-5- yl)benzyl)pyrrolidine- 2-carboxamide | (2S,4R)-1-((S)-3,3- dimethyl-2-(8-oxo-8- (4-(1-(5-(4-(4-((E)-3- (pyridin-3- yl)acrylamido)butyl) piperidine-1- carbonyl)pyridin-2- yl)piperidin-4- yl)piperazin-1- yl)octanamido) butanoyl)-4- hydroxy-N-(4-(4- methylthiazol-5- yl)benzyl)pyrrolidine- 2-carboxamide |
| | 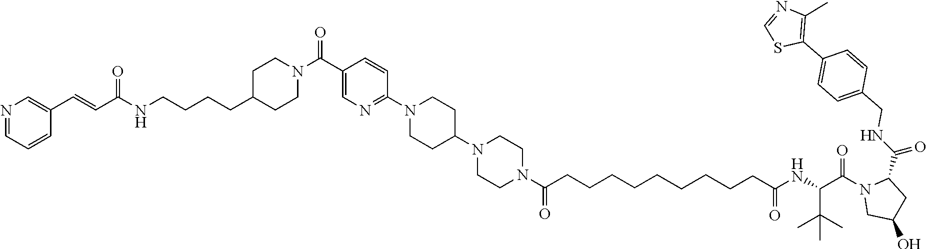 [SMILES: Cc1ncsc1-c1ccc(CNC(=O)[C@@H]2C[C@@H](O)CN2C(=O)[C@@H](NC(=O)CCCCCCCCCC(=O)N2CCN(C3CCN(c4ccc(C(=O)N5CCC(CCCCNC(=O)/C=C/c6cccnc6)CC5)cn4)CC3)CC2)C(C)(C)C)cc1] | (2S,4R)-1-((S)-3,3- dimethyl-2-(11-oxo- 11-(4-(1-(5-(4-(4- ((E)-3-(pyridin-3- yl)acrylamido)butyl) piperidine-1- carbonyl)pyridin-2- yl)piperidin-4- yl)piperazin-1- yl)undecanamido) butanoyl)-4- hydroxy-N-(4-(4- methylthiazol-5- yl)benzyl)pyrrolidine- 2-carboxamide | (2S,4R)-1-((S)-3,3- dimethyl-2-(11-oxo- 11-(4-(1-(5-(4-(4- ((E)-3-(pyridin-3- yl)acrylamido)butyl) piperidine-1- carbonyl)pyridin-2- yl)piperidin-4- yl)piperazin-1- yl)undecanamido) butanoyl)-4- hydroxy-N-(4- (4-methylthiazol-5- yl)benzyl)pyrrolidine- 2-carboxamide |
| | 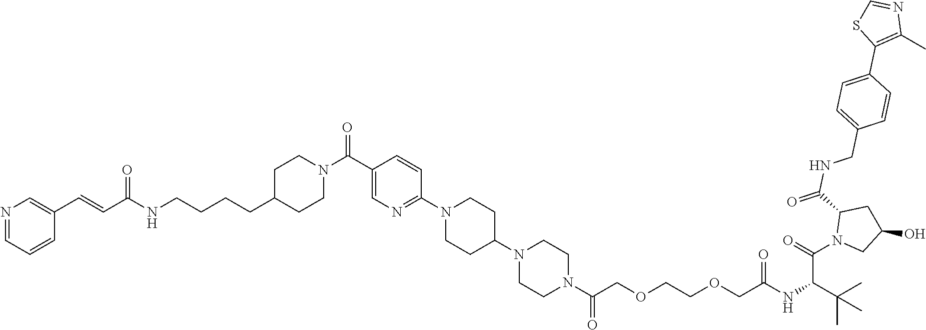 [SMILES: Cc1ncsc1-c1ccc(CNC(=O)[C@@H]2C[C@@H](O)CN2C(=O)[C@@H](NC(=O)COCCOCC(=O)N2CCN(C3CCN(c4ccc(C(=O)N5CCC(CCCCNC(=O)/C=C/c6cccnc6)CC5)cn4)CC3)CC2)C(C)(C)C)cc1] | (2S,4R)-1-((S)-3,3- dimethyl-2-(2-(2-(2- ox0-2-(4-(1-(5-(4-(4- ((E)-3-(pyridin-3- yl)acrylamido)butyl) piperidine-1- carbonyl)pyridin-2- yl)piperidin-4- yl)piperazin-1- yl)ethoxy)ethoxy)aceta mido)butanoyl)-4- hydroxy-N-(4-(4- methylthiazol-5- yl)benzyl)pyrrolidine- 2-carboxamide | (2S,4R)-1-((S)-3,3- dimethyl-2-(2-(2-(2- ox0-2-(4-(1-(5-(4-(4- ((E)-3-(pyridin-3- yl)acrylamido)butyl) piperidine-1- carbonyl)pyridin-2- yl)piperidin-4- yl)piperazin-1- yl)ethoxy)ethoxy) acetamido)butanoyl)- 4-hydroxy-N-(4-(4- methylthiazol-5- yl)benzyl)pyrrolidine- 2-carboxamide |
| | 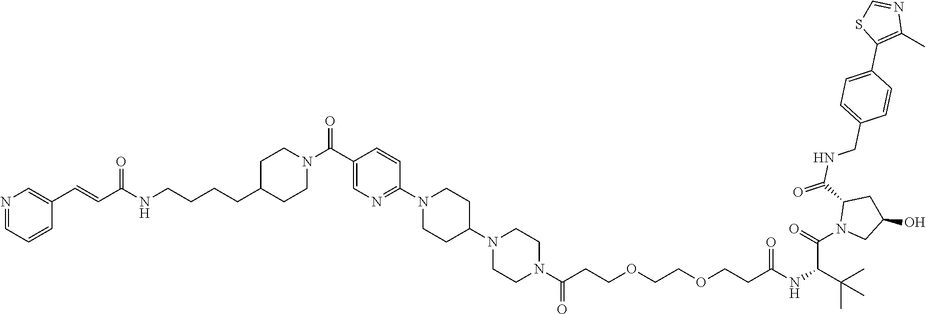 [SMILES: Cc1ncsc1-c1ccc(CNC(=O)[C@@H]2C[C@@H](O)CN2C(=O)[C@@H](NC(=O)CCOCCOCCC(=O)N2CCN(C3CCN(c4ccc(C(=O)N5CCC(CCCCNC(=O)/C=C/c6cccnc6)CC5)cn4)CC3)CC2)C(C)(C)C)cc1] | (2S,4R)-1-((S)-3,3- dimethyl-2-(2-(2-(2- oxo-2-(4-(1-(5-(4-(4- ((E)-3-(pyridin-3- yl)acrylamido)butyl) piperidine-1- carbonyl)pyridin-2- yl)piperidin-4- yl)piperazin-1- yl)ethoxy)ethoxy) acetamido)butanoyl)- 4-hydroxy-N-(4-(4- methylthiazol-5- yl)benzyl)pyrrolidine- 2-carboxamide | (2S,4R)-1-((S)-3,3- dimethyl-2-(2-(2-(2- oxo-2-(4-(1-(5-(4-(4- ((E)-3-(pyridin-3- yl)acrylamido)butyl)pi peridine-1- carbonyl)pyridin-2- yl)piperidin-4- yl)piperazin-1- yl)ethoxy)ethoxy)aceta mido)butanoyl)-4- hydroxy-N-(4-(4- methylthiazol-5- yl)benzyl)pyrrolidine- 2-carboxamide |
| | 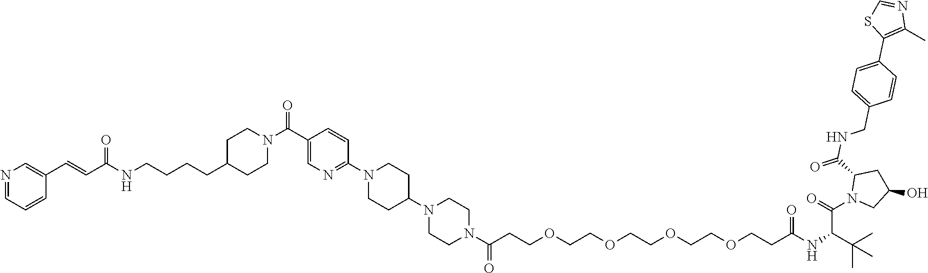 [SMILES: Cc1ncsc1-c1ccc(CNC(=O)[C@@H]2C[C@@H](O)CN2C(=O)[C@@H](NC(=O)CCOCCOCCOCCOCCC(=O)N2CCN(C3CCN(c4ccc(C(=O)N5CCC(CCCCNC(=O)/C=C/c6cccnc6)CC5)cn4)CC3)CC2)C(C)(C)C)cc1] | (2S,4R)-1-((S)-2-(tert- butyl)-4, 19-dioxo-19- (4-(1-(5-(4-(4-((E)-3- (pyridin-3- yl)acrylamido)butyl) piperidine-1- carbonyl)pyridin-2- yl)piperidin-4- yl)piperazin-1-yl)- 7,10,13,16-tetraoxa- 3-azanonadecanoyl)- 4-hydroxy-N-(4-(4- methylthiazol-5- yl)benzyl)pyrrolidine- 2-carboxamide | (2S,4R)-1-((S)-2-(tert- butyl)-4,19-dioxo-19- (4-(1-(5-(4-(4-((E)-3- (pyridin-3- yl)acrylamido)butyl) piperidine-1- carbonyl)pyridin-2- yl)piperidin-4- yl)piperazin-1-yl)- 7,10,13,16-tetraoxa- 3-azanonadecanoyl)- 4- hydroxy-N-(4-(4- methylthiazol-5- yl)benzyl)pyrrolidine- 2-carboxamide |
| | 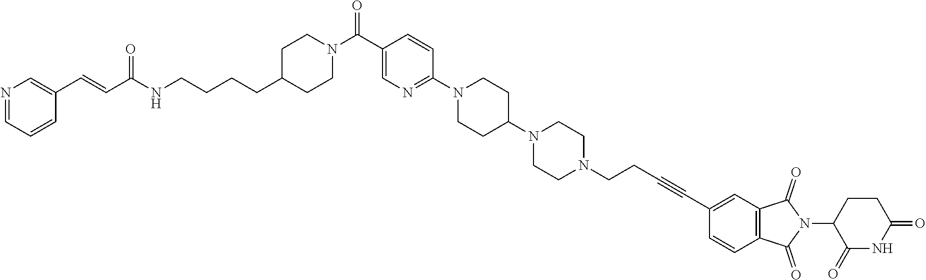 [SMILES: O=C(/C=C/c1cccnc1)NCCCCC1CCN(C(=O)c2ccc(N3CCC(N4CCN(CCC#Cc5ccc6c(c5)C(=O)N(C5CCC(=O)NC5=O)C6=O)CC4)CC3)nc2)CC1] | (E)-N-(4-(1-(6-(4-(4-(4- (2-(2,6-dioxopiperidin- 3-yl)-1,3- dioxoisoindolin-5- yl)but-3-yn-1- yl)piperazin-1- yl)piperidin-1- yl)nicotinoyl) piperidin- 4-yl)butyl)-3- (pyridin-3- yl)acrylamide | (E)-N-(4-(1-(6-(4-(4- (4-(2-(2,6- dioxopiperidin-3-yl)- 1,3-dioxoisoindolin- 5-yl)but-3-yn-1- yl)piperazin-1- yl)piperidin-1- yl)nicotinoyl) piperidin- 4-yl)butyl)- 3-(pyridin- 3-yl)acrylamide |
| | 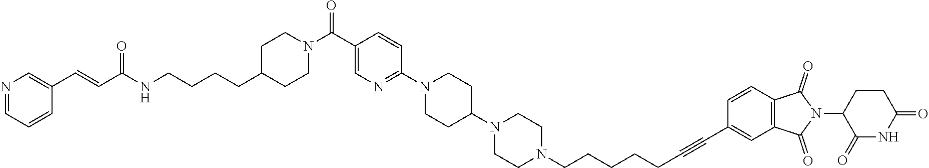 [SMILES: O=C(/C=C/c1cccnc1)NCCCCC1CCN(C(=O)c2ccc(N3CCC(N4CCN(CCCCCC#Cc5ccc6c(c5)C(=O)N(C5CCC(=O)NC5=O)C6=O)CC4)CC3)nc2)CC1] | (E)-N-(4-(1-(6-(4-(4-(7- (2-(2,6-dioxopiperidin- 3-yl)-1,3- dioxoisoindolin-5- yl)hept-6-yn-1- yl)piperazin-1- yl)piperidin-1- yl)nicotinoyl) piperidin-4-yl)butyl)- 3-(pyridin-3- yl)acrylamide | (E)-N-(4-(1-(6-(4-(4- (7-(2-(2,6- dioxopiperidin-3-yl)- 1,3-dioxoisoindolin- 5-yl)hept-6-yn-1- yl)piperazin-1- yl)piperidin-1- yl)nicotinoyl) piperidin-4-yl)butyl)- 3-(pyridin- 3-yl)acrylamide |
| | 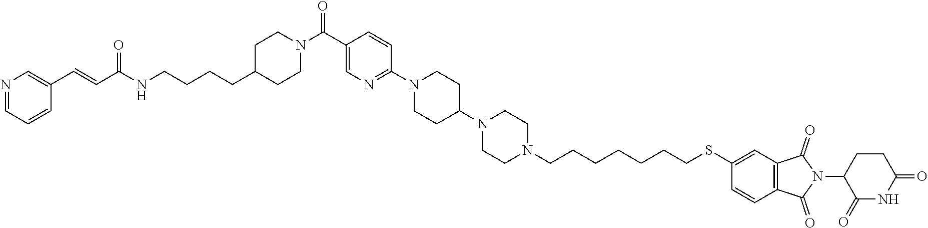 [SMILES: O=C(/C=C/c1cccnc1)NCCCCC1CCN(C(=O)c2ccc(N3CCC(N4CCN(CCCCCCCSc5ccc6c(c5)C(=O)N(C5CCC(=O)NC5=O)C6=O)CC4)CC3)nc2)CC1] | (E)-N-(4-(1-(6-(4-(4-(7- ((2-(2,6-dioxopiperidin- 3-yl)-1,3- dioxoisoindolin-5- yl)thio)heptyl)piperazin- 1-yl)piperidin-1- yl)nicotinoyl)piperidin- 4-yl)butyl)-3-(pyridin- 3-yl)acrylamide | (E)-N-(4-(1-(6-(4-(4- (7-((2-(2,6- dioxopiperidin-3-yl)- 1,3-dioxoisoindolin- 5-yl)thio)heptyl) piperazin-1- yl)piperidin-1- yl)nicotinoyl) piperidin-4-yl) butyl)-3-(pyridin- 3-yl)acrylamide |
| | 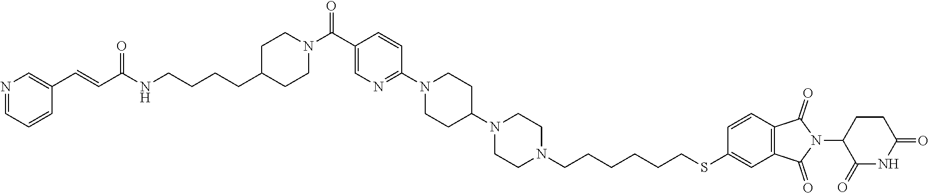 [SMILES: O=C(/C=C/c1cccnc1)NCCCCC1CCN(C(=O)c2ccc(N3CCC(N4CCN(CCCCCCSc5ccc6c(c5)C(=O)N(C5CCC(=O)NC5=O)C6=O)CC4)CC3)nc2)CC1] | (E)-N-(4-(1-(6-(4-(4-(6- ((2-(2,6-dioxopiperidin- 3-yl)-1,3- dioxoisoindolin-5- yl)thio)hexyl) piperazin- 1-yl)piperidin-1- yl)nicotinoyl) piperidin- 4-yl)butyl)-3- (pyridin-3- yl)acrylamide | (E)-N-(4-(1-(6-(4-(4- (6-((2-(2,6- dioxopiperidin-3-yl)- 1,3-dioxoisoindolin- 5-yl)thio)hexyl) piperazin-1- yl)piperidin-1- yl)nicotinoyl) piperidin-4-yl) butyl)-3-(pyridin- 3-yl)acrylamide |
| | 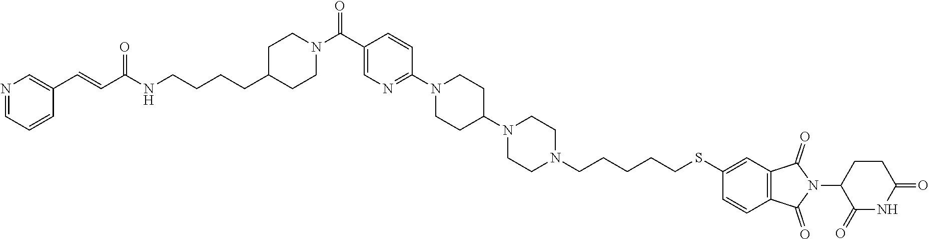 [SMILES: O=C(/C=C/c1cccnc1)NCCCCC1CCN(C(=O)c2ccc(N3CCC(N4CCN(CCCCCSc5ccc6c(c5)C(=O)N(C5CCC(=O)NC5=O)C6=O)CC4)CC3)nc2)CC1] | (E)-N-(4-(1-(6-(4-(4-(5- ((2-(2,6-dioxopiperidin- 3-yl)-1,3- dioxoisoindolin- 5-yl)thio)pentyl) piperazin- 1-yl)piperidin-1- yl)nicotinoyl) piperidin- 4-yl)butyl)- 3-(pyridin- 3-yl)acrylamide | (E)-N-(4-(1-(6-(4-(4- (5-((2-(2,6- dioxopiperidin-3- yl)-1,3- dioxoisoindolin-5- yl)thio)pentyl) piperazin-1- yl)piperidin-1- yl)nicotinoyl) piperidin- 4-yl)butyl)-3- (pyridin- 3-yl)acrylamide |
| | 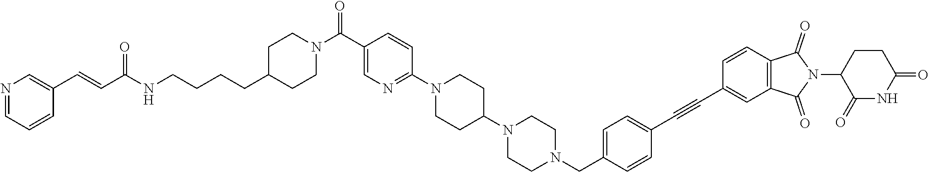 [SMILES: O=C(/C=C/c1cccnc1)NCCCCC1CCN(C(=O)c2ccc(N3CCC(N4CCN(Cc5ccc(C#Cc6ccc7c(c6)C(=O)N(C6CCC(=O)NC6=O)C7=O)cc5)CC4)CC3)nc2)CC1] | (E)-N-(4-(1-(6-(4-(4-(4- ((2-(2,6-dioxopiperidin- 3-yl)-1,3- dioxoisoindolin-5- yl)ethynyl)benzyl) piperazin-1- yl)piperidin-1- yl)nicotinoyl)piperidin- 4-yl)butyl)-3-(pyridin- 3-yl)acrylamide | (E)-N-(4-(1-(6-(4-(4- (4-((2-(2,6- dioxopiperidin-3-yl)- 1,3-dioxoisoindolin- 5-yl)ethynyl)benzyl) piperazin-1- yl)piperidin-1- yl)nicotinoyl) piperidin-4- yl)butyl)-3-(pyridin- 3-yl)acrylamide |
| | 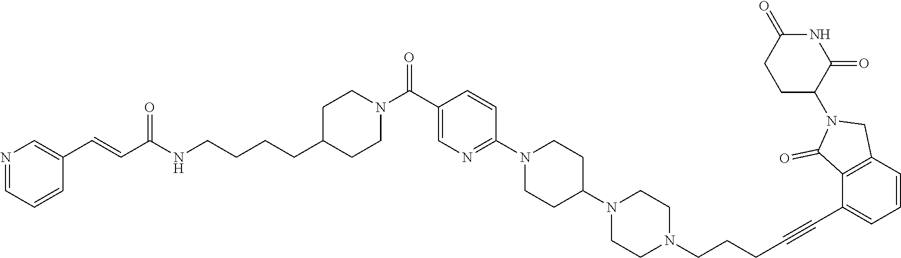 [SMILES: O=C(/C=C/c1cccnc1)NCCCCC1CCN(C(=O)c2ccc(N3CCC(N4CCN(CCCC#Cc5cccc6c5C(=O)N(C5CCC(=O)NC5=O)C6)CC4)CC3)nc2)CC1] | (E)-N-(4-(1-(6-(4-(4-(5- (2-(2,6-dioxopiperidin- 3-yl)-3-oxoisoindolin- 4-yl)pent-4-yn-1- yl)piperazin-1- yl)piperidin-1- yl)nicotinoyl)piperidin- 4-yl)butyl)-3-(pyridin- 3-yl)acrylamide | (E)-N-(4-(1-(6-(4-(4- (5-(2-(2,6- dioxopiperidin-3-yl)- 3-oxoisoindolin-4- yl)pent-4-yn-1- yl)piperazin-1- yl)piperidin-1- yl)nicotinoyl) piperidin-4- yl)butyl)-3-(pyridin- 3-yl)acrylamide |
| | 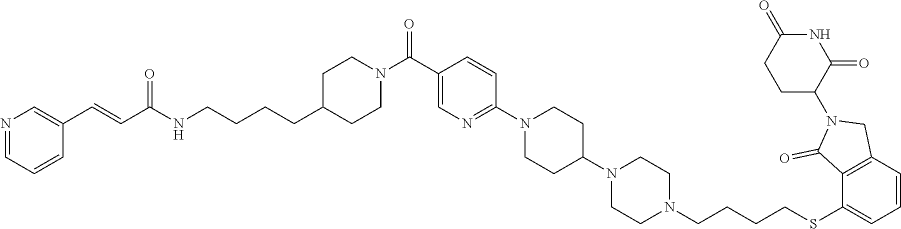 [SMILES: O=C(/C=C/c1cccnc1)NCCCCC1CCN(C(=O)c2ccc(N3CCC(N4CCN(CCCCSc5cccc6c5C(=O)N(C5CCC(=O)NC5=O)C6)CC4)CC3)nc2)CC1] | (E)-N-(4-(1-(6-(4-(4-(4- ((2-(2,6-dioxopiperidin- 3-yl)-3-oxoisoindolin- 4-yl)thio)butyl) piperazin-1- yl)piperidin-1- yl)nicotinoyl)piperidin- 4-yl)butyl)-3-(pyridin- 3-yl)acrylamide | (E)-N-(4-(1-(6-(4-(4- (4-((2-(2,6- dioxopiperidin-3- yl)-3-oxoisoindolin- 4-yl)thio)butyl) piperazin- 1-yl)piperidin-1- yl)nicotinoyl) piperidin-4-yl) butyl)-3-(pyridin- 3-yl)acrylamide |
| | 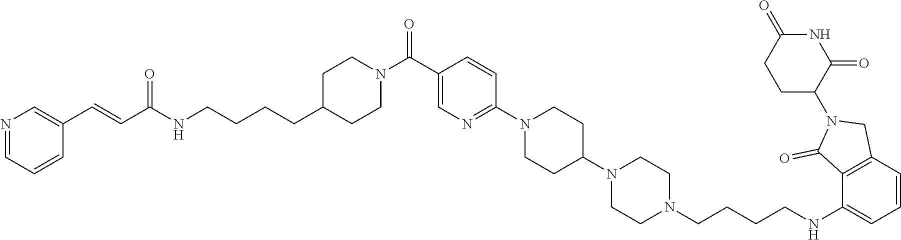 [SMILES: O=C(/C=C/c1cccnc1)NCCCCC1CCN(C(=O)c2ccc(N3CCC(N4CCN(CCCCNc5cccc6c5C(=O)N(C5CCC(=O)NC5=O)C6)CC4)CC3)nc2)CC1] | (E)-N-(4-(1-(6-(4-(4-(4- ((2-(2,6-dioxopiperidin- 3-yl)-3-oxoisoindolin- 4-yl)amino)butyl) piperazin-1- yl)piperidin-1- yl)nicotinoyl)piperidin- 4-yl)butyl)-3-(pyridin- 3-yl)acrylamide | (E)-N-(4-(1-(6-(4-(4- (4-((2-(2,6- dioxopiperidin-3-yl)- 3-oxoisoindolin-4- yl)amino)butyl) piperazin-1- yl)piperidin-1- yl)nicotinoyl) piperidin-4- yl)butyl)-3-(pyridin- 3-yl)acrylamide |
| | 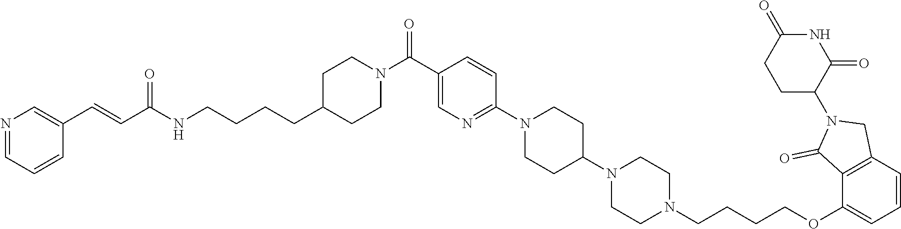 [SMILES: O=C(/C=C/c1cccnc1)NCCCCC1CCN(C(=O)c2ccc(N3CCC(N4CCN(CCCCOc5cccc6c5C(=O)N(C5CCC(=O)NC5=O)C6)CC4)CC3)nc2)CC1] | (E)-N-(4-(1-(6-(4-(4-(4- ((2-(2,6-dioxopiperidin- 3-yl)-3-oxoisoindolin- 4-yl)oxy)butyl) piperazin- 1-yl)piperidin-1- yl)nicotinoyl)piperidin- 4-yl)butyl)-3-(pyridin- 3-yl)acrylamide | (E)-N-(4-(1-(6-(4-(4- (4-((2-(2,6- dioxopiperidin-3-yl)- 3-oxoisoindolin-4- yl)oxy)butyl) piperazin-1- yl)piperidin-1- yl)nicotinoyl) piperidin-4- yl)butyl)-3- (pyridin- 3-yl)acrylamide |
| | 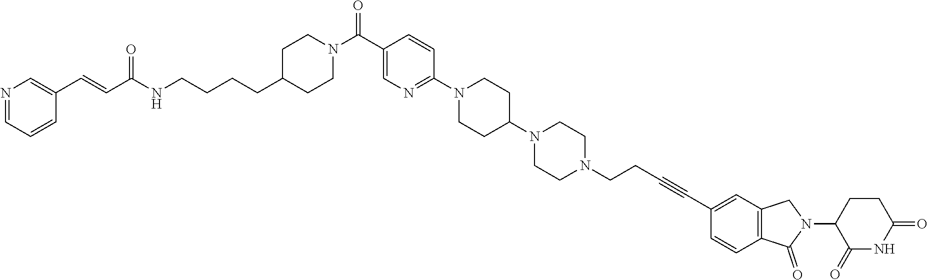 [SMILES: O=C(/C=C/c1cccnc1)NCCCCC1CCN(C(=O)c2ccc(N3CCC(N4CCN(CCC#Cc5ccc6c(c5)CN(C5CCC(=O)NC5=O)C6=O)CC4)CC3)nc2)CC1] | (E)-N-(4-(1-(6-(4-(4-(4- (2-(2,6-dioxopiperidin- 3-yl)-1-oxoisoindolin- 5-yl)but-3-yn-1- yl)piperazin-1- yl)piperidin-1- yl)nicotinoyl)piperidin- 4-yl)butyl)-3-(pyridin- 3-yl)acrylamide | (E)-N-(4-(1-(6-(4-(4- (4-(2-(2,6- dioxopiperidin-3-yl)- 1-oxoisoindolin- 5-yl)but-3-yn-1- yl)piperazin-1- yl)piperidin-1 - yl)nicotinoyl) piperidin- 4-yl)butyl)- 3-(pyridin- 3-yl)acrylamide |
| | 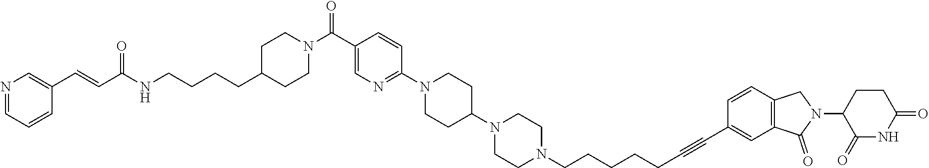 [SMILES: O=C(/C=C/c1cccnc1)NCCCCC1CCN(C(=O)c2ccc(N3CCC(N4CCN(CCCCCC#Cc5ccc6c(c5)C(=O)N(C5CCC(=O)NC5=O)C6)CC4)CC3)nc2)CC1] | (E)-N-(4-(1-(6-(4-(4-(7- (2-(2,6-dioxopiperidin- 3-yl)-3-oxoisoindolin- 5-yl)hept-6-yn-1- yl)piperazin-1- yl)piperidin-1- yl)nicotinoyl)piperidin- 4-yl)butyl)-3-(pyridin- 3-yl)acrylamide | (E)-N-(4-(1-(6-(4-(4- (7-(2-(2,6- dioxopiperidin-3-yl)- 3-oxoisoindolin-5- yl)hept-6-yn-1- yl)piperazin-1- yl)piperidin-1- yl)nicotinoyl) piperidin-4-yl) butyl)-3-(pyridin- 3-yl)acrylamide |
| | 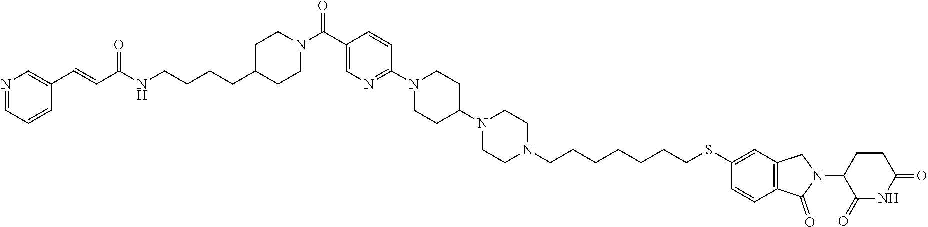 [SMILES: O=C(/C=C/c1cccnc1)NCCCCC1CCN(C(=O)c2ccc(N3CCC(N4CCN(CCCCCCCSc5ccc6c(c5)CN(C5CCC(=O)NC5=O)C6=O)CC4)CC3)nc2)CC1] | (E)-N-(4-(1-(6-(4-(4-(7- ((2-(2,6-dioxopiperidin- 3-yl)-1-oxoisoindolin- 5-yl)thio)heptyl) piperazin- 1-yl)piperidin-1- yl)nicotinoyl)piperidin- 4-yl)butyl)-3-(pyridin- 3-yl)acrylamide | (E)-N-(4-(1-(6-(4-(4- (7-((2-(2,6- dioxopiperidin-3-yl)- 1-oxoisoindolin-5- yl)thio)heptyl) piperazin-1- yl)piperidin-1- yl)nicotinoyl) piperidin- 4-yl)butyl)- 3-(pyridin- 3-yl)acrylamide |
| | 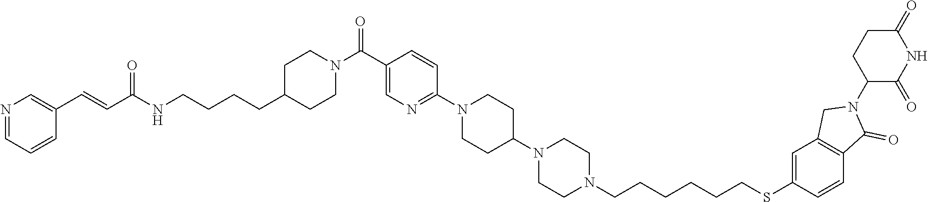 [SMILES: O=C(/C=C/c1cccnc1)NCCCCC1CCN(C(=O)c2ccc(N3CCC(N4CCN(CCCCCCSc5ccc6c(c5)CN(C5CCC(=O)NC5=O)C6=O)CC4)CC3)nc2)CC1] | (E)-N-(4-(1-(6-(4-(4-(6- ((2-(2,6-dioxopiperidin- 3-yl)-1-oxoisoindolin- 5-yl)thio)hexyl) piperazin-1- yl)piperidin-1- yl)nicotinoyl)piperidin- 4-yl)butyl)-3-(pyridin- 3-yl)acrylamide | (E)-N-(4-(1-(6-(4-(4- (6-((2-(2,6- dioxopiperidin-3-yl)- 1-oxoisoindolin-5- yl)thio)hexyl) piperazin-1- yl)piperidin-1- yl)nicotinoyl) piperidin-4- yl)butyl)-3-(pyridin- 3-yl)acrylamide |
| | 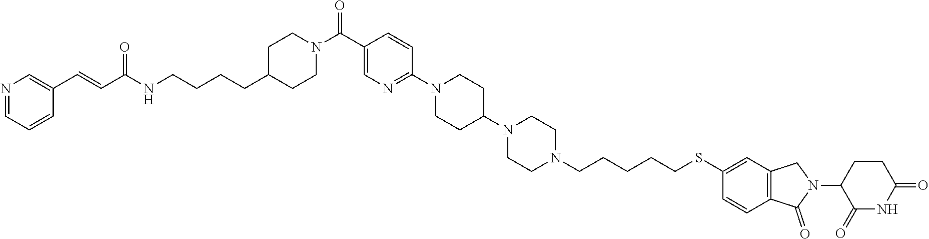 [SMILES: O=C(/C=C/c1cccnc1)NCCCCC1CCN(C(=O)c2ccc(N3CCC(N4CCN(CCCCCSc5ccc6c(c5)CN(C5CCC(=O)NC5=O)C6=O)CC4)CC3)nc2)CC1] | (E)-N-(4-(1-(6-(4-(4-(5- ((2-(2,6-dioxopiperidin- 3-yl)-1-oxoisoindolin- 5-yl)thio)pentyl) piperazin- 1-yl)piperidin-1- yl)nicotinoyl)piperidin- 4-yl)butyl)-3-(pyridin- 3-yl)acrylamide | (E)-N-(4-(1-(6-(4-(4- (5-((2-(2,6- dioxopiperidin-3- yl)-1 - oxoisoindolin-5- yl)thio)pentyl) piperazin-1- yl)piperidin-1- yl)nicotinoyl) piperidin- 4-yl)butyl)- 3-(pyridin- 3-yl)acrylamide |
| | 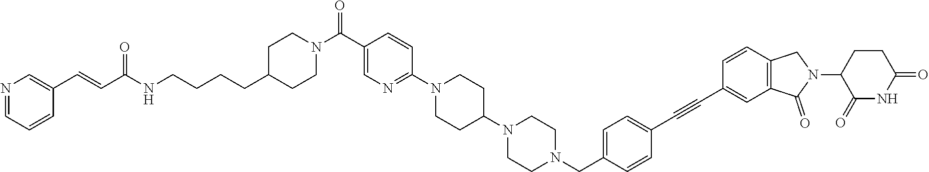 [SMILES: O=C(/C=C/c1cccnc1)NCCCCC1CCN(C(=O)c2ccc(N3CCC(N4CCN(Cc5ccc(C#Cc6ccc7c(c6)C(=O)N(C6CCC(=O)NC6=O)C7)cc5)CC4)CC3)nc2)CC1] | (E)-N-(4-(1-(6-(4-(4-(4- ((2-(2,6-dioxopiperidin- 3-yl)-3-oxoisoindolin- 5-yl)ethynyl)benzyl) piperazin-1-yl) piperidin-1- yl)nicotinoyl)piperidin- 4-yl)butyl)-3-(pyridin- 3-yl)acrylamide | (E)-N-(4-(1-(6-(4-(4- (4-((2-(2,6- dioxopiperidin-3-yl)- 3-oxoisoindolin-5- yl)ethynyl)benzyl) piperazin-1-yl) piperidin-1- yl)nicotinoyl) piperidin-4-yl)butyl)- 3-(pyridin- 3-yl)acrylamide |
| | 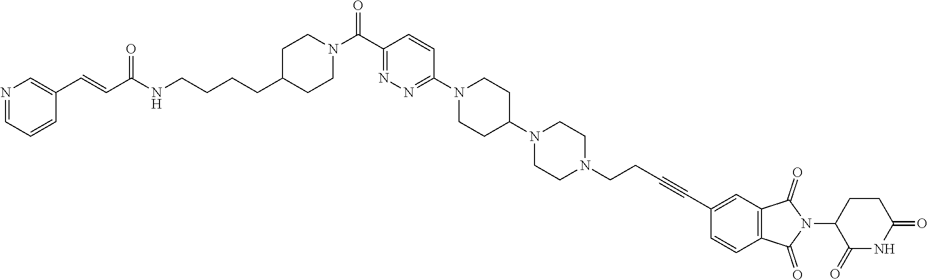 [SMILES: O=C(/C=C/c1cccnc1)NCCCCC1CCN(C(=O)c2ccc(N3CCC(N4CCN(CCC#Cc5ccc6c(c5)C(=O)N(C5CCC(=O)NC5=O)C6=O)CC4)CC3)nn2)CC1] | (E)-N-(4-(1-(6-(4-(4-(4- (2-(2,6-dioxopiperidin- 3-yl)-1,3- dioxoisoindolin-5- yl)but-3-yn-1- yl)piperazin-1- yl)piperidin-1- yl)pyridazine-3- carbonyl)piperidin-4- yl)butyl)-3-(pyridin- 3-yl)acrylamide | (E)-N-(4-(1-(6-(4-(4- (4-(2-(2,6- dioxopiperidin-3-yl)- 1,3-dioxoisoindolin- 5-yl)but-3-yn-1- yl)piperazin-1- yl)piperidin-1- yl)pyridazine-3- carbonyl)piperidin- 4-yl)butyl)-3- (pyridin-3- yl)acrylamide |
| | 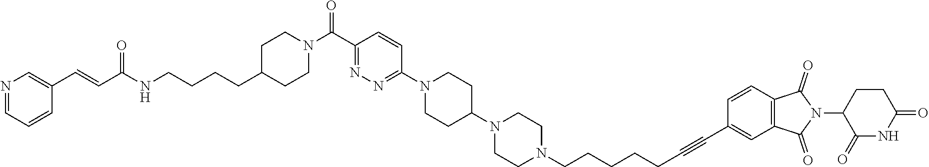 [SMILES: O=C(/C=C/c1cccnc1)NCCCCC1CCN(C(=O)c2ccc(N3CCC(N4CCN(CCCCCC#Cc5ccc6c(c5)C(=O)N(C5CCC(=O)NC5=O)C6=O)CC4)CC3)nn2)CC1] | (E)-N-(4-(1-(6-(4-(4-(7- (2-(2,6-dioxopiperidin- 3-yl)-1,3- dioxoisoindolin-5- yl)hept-6-yn-1- yl)piperazin-1- yl)piperidin-1- yl)pyridazine-3- carbonyl)piperidin-4- yl)butyl)-3-(pyridin- 3-yl)acrylamide | (E)-N-(4-(1-(6-(4-(4- (7-(2-(2,6- dioxopiperidin-3-yl)- 1,3-dioxoisoindolin- 5-yl)hept-6-yn-1- yl)piperazin-1- yl)piperidin-1- yl)pyridazine-3- carbonyl)piperidin-4- yl)butyl)-3-(pyridin- 3-yl)acrylamide |
| | 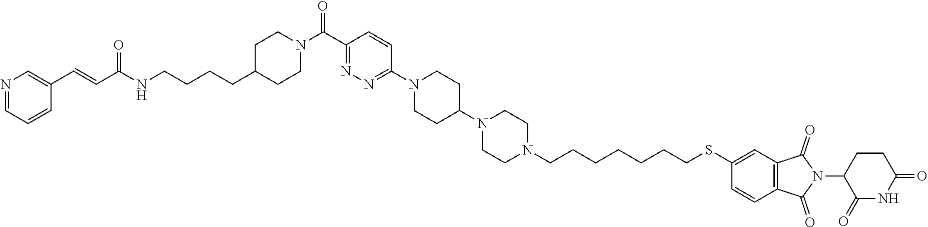 [SMILES: O=C(/C=C/c1cccnc1)NCCCCC1CCN(C(=O)c2ccc(N3CCC(N4CCN(CCCCCCCSc5ccc6c(c5)C(=O)N(C5CCC(=O)NC5=O)C6=O)CC4)CC3)nn2)CC1] | (E)-N-(4-(1-(6-(4-(4-(7- ((2-(2,6-dioxopiperidin- 3-yl)-1,3- dioxoisoindolin-5- yl)thio)heptyl) piperazin- 1-yl)piperidin-1- yl)pyridazine-3- carbonyl)piperidin-4- yl)butyl)-3-(pyridin- 3-yl)acrylamide | (E)-N-(4-(1-(6-(4-(4- (7-((2-(2,6- dioxopiperidin-3-yl)- 1,3-dioxoisoindolin- 5-yl)thio)heptyl) piperazin-1- yl)piperidin-1- yl)pyridazine-3- carbonyl)piperidin- 4-yl)butyl)- 3-(pyridin-3- yl)acrylamide |
| | 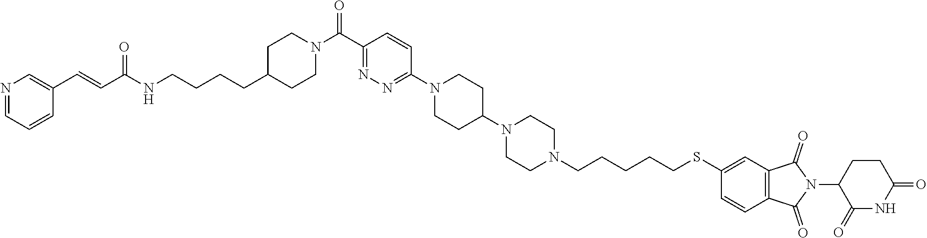 [SMILES: O=C(/C=C/c1cccnc1)NCCCCC1CCN(C(=O)c2ccc(N3CCC(N4CCN(CCCCCSc5ccc6c(c5)C(=O)N(C5CCC(=O)NC5=O)C6=O)CC4)CC3)nn2)CC1] | (E)-N-(4-(1-(6-(4-(4-(6- ((2-(2,6-dioxopiperidin- 3-yl)-1,3- dioxoisoindolin-5- yl)thio)hexyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (6-((2-(2,6- dioxopiperidin-3-yl)- 1,3-dioxoisoindolin- 5-yl)thio) hexyl)piperazin- 1-yl)piperidin-1- yl)pyridazine-3- carbonyl)piperidin-4- yl)butyl)-3-(pyridin- 3-yl)acrylamide |
| | 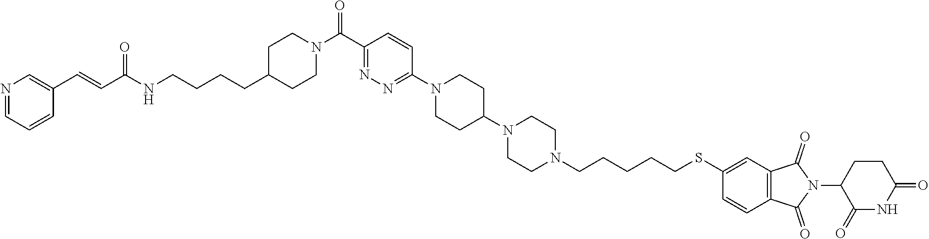 [SMILES: O=C(/C=C/c1cccnc1)NCCCCC1CCN(C(=O)c2ccc(N3CCC(N4CCN(CCCCCSc5ccc6c(c5)C(=O)N(C5CCC(=O)NC5=O)C6=O)CC4)CC3)nn2)CC1] | (E)-N-(4-(1-(6-(4-(4-(5- ((2-(2,6-dioxopiperidin- 3-yl)-1,3- dioxoisoindolin-5- yl)thio)pentyl) piperazin-1- yl)piperidin-1- yl)pyridazine-3- carbonyl)piperidin-4- yl)butyl)-3-(pyridin- 3-yl)acrylamide | (E)-N-(4-(1-(6-(4-(4- (5-((2-(2,6- dioxopiperidin-3-yl)- 1,3-dioxoisoindolin- 5-yl)thio)pentyl) piperazin-1- yl)piperidin-1- yl)pyridazine-3- carbonyl)piperidin- 4-yl)butyl)-3- (pyridin-3- yl)acrylamide |
| | 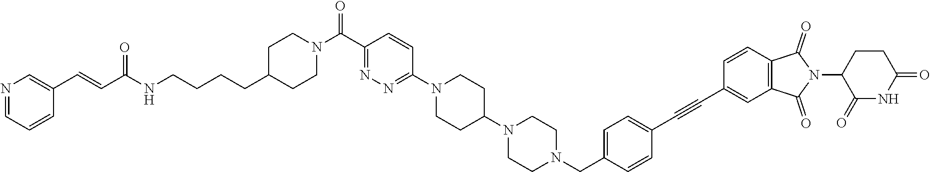 [SMILES: O=C(/C=C/c1cccnc1)NCCCCC1CCN(C(=O)c2ccc(N3CCC(N4CCN(Cc5ccc(C#Cc6ccc7c(c6)C(=O)N(C6CCC(=O)NC6=O)C7=O)cc5)CC4)CC3)nn2)CC1] | (E)-N-(4-(1-(6-(4-(4-(4- ((2-(2,6-dioxopiperidin- 3-yl)-1,3- dioxoisoindolin-5- yl)ethynyl)benzyl) piperazin-1- yl)piperidin-1- yl)pyridazine-3- carbonyl)piperidin-4- yl)butyl)-3-(pyridin- 3-yl)acrylamide | (E)-N-(4-(1-(6-(4-(4- (4-((2-(2,6- dioxopiperidin-3- yl)-1,3- dioxoisoindolin-5- yl)ethynyl)benzyl) piperazin-1- yl)piperidin-1- yl)pyridazine-3- carbonyl)piperidin- 4-yl)butyl)- 3-(pyridin-3- yl)acrylamide |
| | 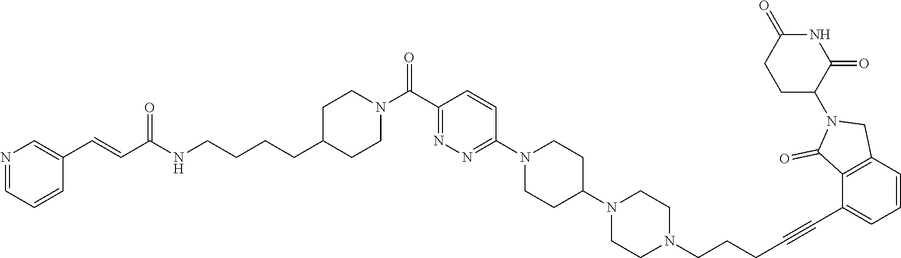 [SMILES: O=C(/C=C/c1cccnc1)NCCCCC1CCN(C(=O)c2ccc(N3CCC(N4CCN(CCCC#Cc5cccc6c5C(=O)N(C5CCC(=O)NC5=O)C6)CC4)CC3)nn2)CC1] | (E)-N-(4-(1-(6-(4-(4-(5- (2-(2,6-dioxopiperidin- 3-yl)-3-oxoisoindolin- 4-yl)pent-4-yn-1- yl)piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (5-(2-(2,6- dioxopiperidin-3-yl)- 3-oxoisoindolin-4- yl)pent-4-yn-1- yl)piperazin-1- yl)piperidin-1- yl)pyridazine-3- carbonyl)piperidin- 4-yl)butyl)- 3-(pyridin-3- yl)acrylamide |
| | 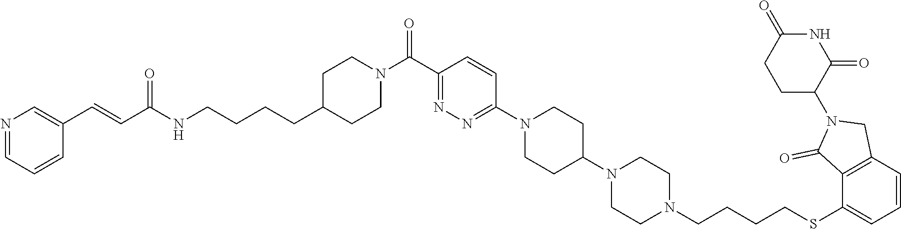 [SMILES: O=C(/C=C/c1cccnc1)NCCCCC1CCN(C(=O)c2ccc(N3CCC(N4CCN(CCCCSc5cccc6c5C(=O)N(C5CCC(=O)NC5=O)C6)CC4)CC3)nn2)CC1] | (E)-N-(4-(1-(6-(4-(4-(4- ((2-(2,6- dioxopiperidin- 3-yl)-3-oxoisoindolin- 4-yl)thio)butyl) piperazin-1- yl)piperidin-1 - yl)pyridazine-3- carbonyl)piperidin-4- yl)butyl)-3-(pyridin-3- yl)acrylamide | (E)-N-(4-(1-(6-(4-(4- (4-((2-(2,6- dioxopiperidin-3-yl)- 3-oxoisoindolin-4- yl)thio)butyl) piperazin-1-yl) piperidin-1- yl)pyridazine-3- carbonyl)piperidin- 4-yl)butyl)-3- (pyridin-3- yl)acrylamide |
| | 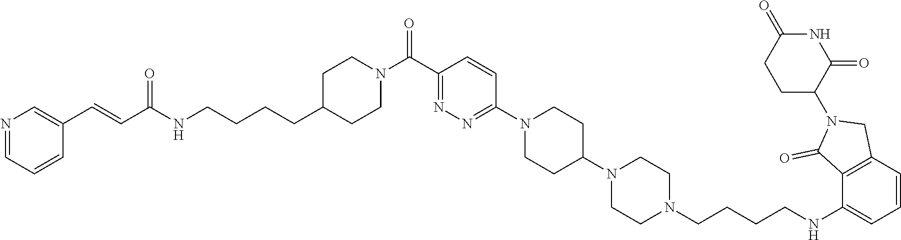 [SMILES: O=C(/C=C/c1cccnc1)NCCCCC1CCN(C(=O)c2ccc(N3CCC(N4CCN(CCCCNc5cccc6c5C(=O)N(C5CCC(=O)NC5=O)C6)CC4)CC3)nn2)CC1] | (E)-N-(4-(1-(6-(4-(4-(4- ((2-(2,6-dioxopiperidin- 3-yl)-3-oxoisoindolin- 4-yl)amino)butyl) piperazin-1- yl)piperidin-1- yl)pyridazine-3- carbonyl)piperidin-4- yl)butyl)-3-(pyridin- 3-yl)acrylamide | (E)-N-(4-(1-(6-(4-(4- (4-((2-(2,6- dioxopiperidin-3- yl)-3-oxoisoindolin- 4-yl)amino) butyl)piperazi n-1-yl)piperidin-1- yl)pyridazine-3- carbonyl)piperidin- 4-yl)butyl)- 3-(pyridin-3- yl)acrylamide |
| | 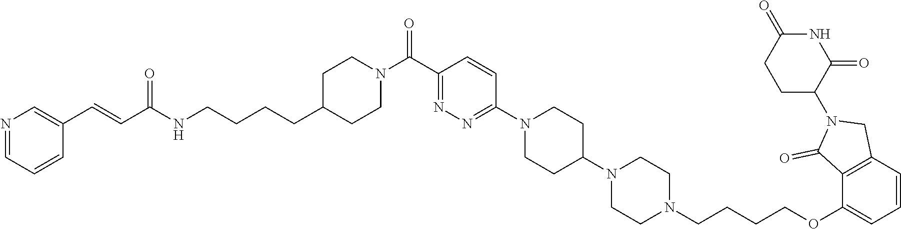 [SMILES: O=C(/C=C/c1cccnc1)NCCCCC1CCN(C(=O)c2ccc(N3CCC(N4CCN(CCCCOc5cccc6c5C(=O)N(C5CCC(=O)NC5=O)C6)CC4)CC3)nn2)CC1] | (E)-N-(4-(1-(6-(4-(4-(4- ((2-(2,6-dioxopiperidin- 3-yl)-3-oxoisoindolin-4- yl)oxy)butyl)piperazin- 1-yl)piperidin-1- yl)pyridazine-3- carbonyl)piperidin-4- yl)butyl)-3-(pyridin-3- yl)acrylamide | (E)-N-(4-(1-(6-(4-(4- (4-((2-(2,6- dioxopiperidin-3-yl)- 3-oxoisoindolin-4- yl)oxy)butyl) piperazin- 1-yl)piperidin-1- yl)pyridazine-3- carbonyl)piperidin- 4-yl)butyl)-3- (pyridin-3- yl)acrylamide |
| | 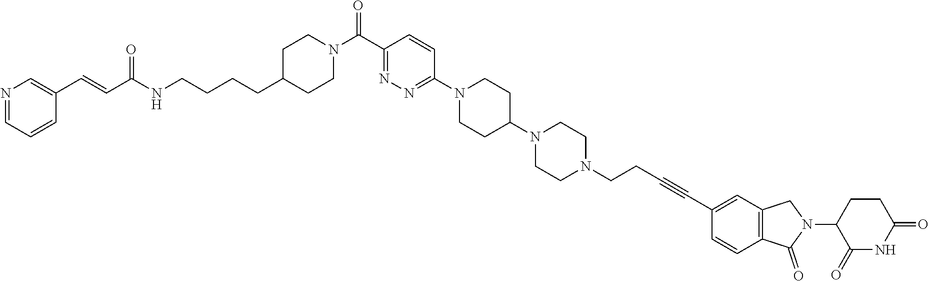 [SMILES: O=C(/C=C/c1cccnc1)NCCCCC1CCN(C(=O)c2ccc(N3CCC(N4CCN(CCC#Cc5ccc6c(c5)CN(C5CCC(=O)NC5=O)C6=O)CC4)CC3)nn2)CC1] | (E)-N-(4-(1-(6-(4-(4-(4- (2-(2,6-dioxopiperidin- 3-yl)-1-oxoisoindolin- 5-yl)but-3-yn-1- yl)piperazin-1- yl)piperidin-1- yl)pyridazine-3- carbonyl)piperidin-4- yl)butyl)-3-(pyridin- 3-yl)acrylamide | (E)-N-(4-(1-(6-(4-(4- (4-(2-(2,6- dioxopiperidin-3-yl)- 1-oxoisoindolin-5- yl)but-3-yn-1-yl) piperazin-1- yl)piperidin-1- yl)pyridazine-3- carbonyl)piperidin-4- yl)butyl)-3-(pyridin- 3-yl)acrylamide |
| | 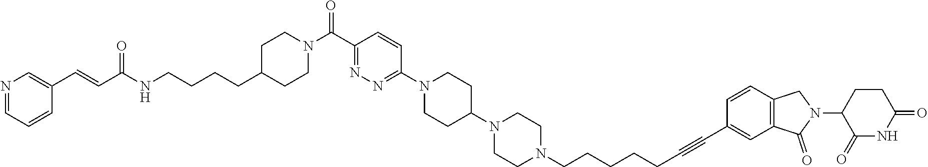 [SMILES: O=C(/C=C/c1cccnc1)NCCCCC1CCN(C(=O)c2ccc(N3CCC(N4CCN(CCCCCC#Cc5ccc6c(c5)C(=O)N(C5CCC(=O)NC5=O)C6)CC4)CC3)nn2)CC1] | (E)-N-(4-(1-(6-(4-(4-(7- (2-(2,6-dioxopiperidin- 3-yl)-3-oxoisoindolin- 5-yl)hept-6-yn-1- yl)piperazin-1- yl)piperidin-1- yl)pyridazine-3- carbonyl)piperidin-4- yl)butyl)-3-(pyridin- 3-yl)acrylamide | (E)-N-(4-(1-(6-(4-(4- (7-(2-(2,6- dioxopiperidin-3- yl)-3-oxoisoindolin- 5-yl)hept-6-yn-1- yl)piperazin-1- yl)piperidin-1- yl)pyridazine-3- carbonyl)piperidin- 4-yl)butyl)-3- (pyridin-3- yl)acrylamide |
| | 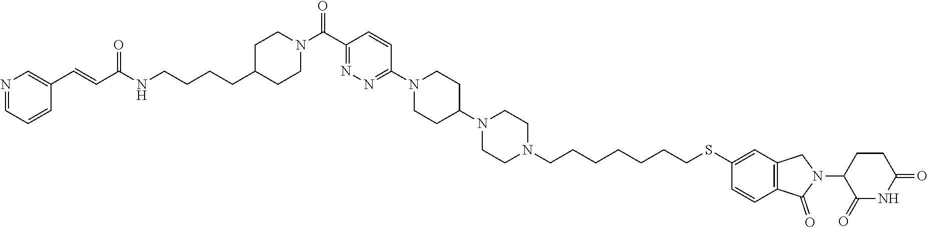 [SMILES: O=C(/C=C/c1cccnc1)NCCCCC1CCN(C(=O)c2ccc(N3CCC(N4CCN(CCCCCCCSc5ccc6c(c5)CN(C5CCC(=O)NC5=O)C6=O)CC4)CC3)nn2)CC1] | (E)-N-(4-(1-(6-(4-(4-(7- ((2-(2,6-dioxopiperidin- 3-yl)-1-oxoisoindolin- 5-yl)thio)heptyl) piperazin-1-yl) piperidin-1- yl)pyridazine-3- carbonyl)piperidin-4- yl)butyl)-3-(pyridin-3- yl)acrylamide | (E)-N-(4-(1-(6-(4-(4- (7-((2-(2,6- dioxopiperidin-3-yl)- 1-oxoisoindolin-5- yl)thio)heptyl) piperazin-1- yl)piperidin-1- yl)pyridazine-3- carbonyl)piperidin-4- yl)butyl)-3-(pyridin- 3-yl)acrylamide |
| | 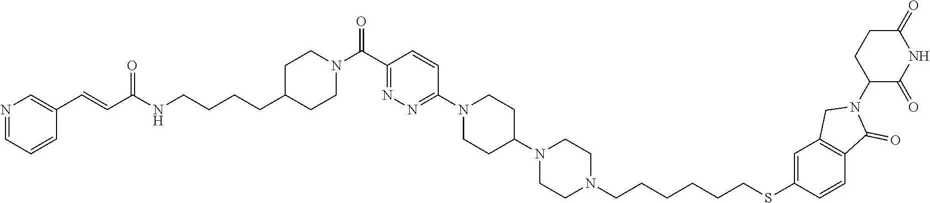 [SMILES: O=C(/C=C/c1cccnc1)NCCCCC1CCN(C(=O)c2ccc(N3CCC(N4CCN(CCCCCCSc5ccc6c(c5)CN(C5CCC(=O)NC5=O)C6=O)CC4)CC3)nn2)CC1] | (E)-N-(4-(1-(6-(4-(4-(6- ((2-(2,6-dioxopiperidin- 3-yl)-1-oxoisoindolin- 5-yl)thio)hexyl) piperazin-1- yl)piperidin-1 - yl)pyridazine-3- carbonyl)piperidin-4- yl)butyl)-3-(pyridin- 3-yl)acrylamide | (E)-N-(4-(1-(6-(4-(4- (6-((2-(2,6- dioxopiperidin-3- yl)-1- oxoisoindolin-5- yl)thio)hexyl) piperazin-1- yl)piperidin-1- yl)pyridazine-3- carbonyl)piperidin- 4-yl)butyl)-3- (pyridin-3- yl)acrylamide |
| | 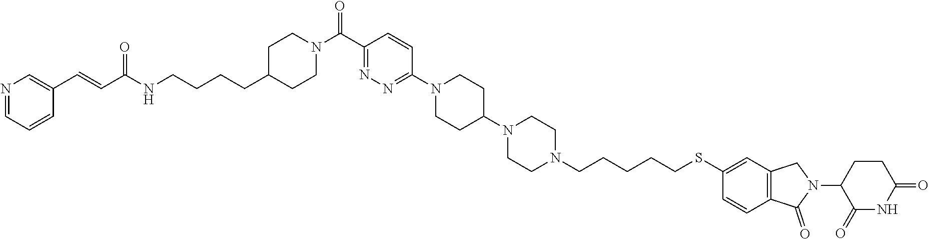 [SMILES: O=C(/C=C/c1cccnc1)NCCCCC1CCN(C(=O)c2ccc(N3CCC(N4CCN(CCCCCSc5ccc6c(c5)CN(C5CCC(=O)NC5=O)C6=O)CC4)CC3)nn2)CC1] | (E)-N-(4-(1-(6-(4-(4-(5- ((2-(2,6-dioxopiperidin- 3-yl)-1-oxoisoindolin- 5-yl)thio)pentyl) piperazin- 1-yl)piperidin-1 - yl)pyridazine-3- carbonyl)piperidin-4- yl)butyl)-3-(pyridin-3- yl)acrylamide | (E)-N-(4-(1-(6-(4-(4- (5-((2-(2,6- dioxopiperidin-3- yl)-1- oxoisoindolin-5- yl)thio)pentyl) piperazin-1- yl)piperidin-1- yl)pyridazine-3- carbonyl)piperidin- 4-yl)butyl)- 3-(pyridin-3- yl)acrylamide |
| | 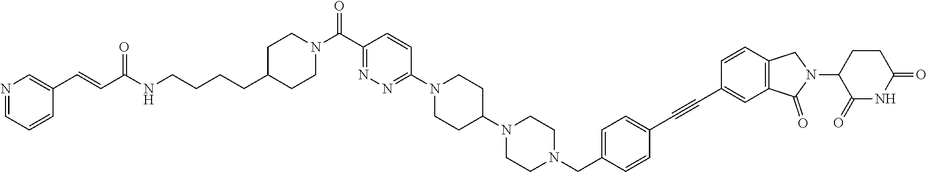 [SMILES: O=C(/C=C/c1cccnc1)NCCCCC1CCN(C(=O)c2ccc(N3CCC(N4CCN(Cc5ccc(C#Cc6ccc7c(c6)C(=O)N(C6CCC(=O)NC6=O)C7)cc5)CC4)CC3)nn2)CC1] | (E)-N-(4-(1-(6-(4-(4-(4- ((2-(2,6-dioxopiperidin- 3-yl)-3-oxoisoindolin- 5-yl)ethynyl)benzyl) piperazin-1- yl)piperidin-1- yl)pyridazine-3- carbonyl)piperidin-4- yl)butyl)-3-(pyridin-3- yl)acrylamide | (E)-N-(4-(1-(6-(4-(4- (4-((2-(2,6- dioxopiperidin-3-yl)- 3-oxoisoindolin-5- yl)ethynyl)benzyl) piperazin-1- yl)piperidin-1- yl)pyridazine-3- carbonyl)piperidin-4- yl)butyl)-3-(pyridin- 3-yl)acrylamide |
| | 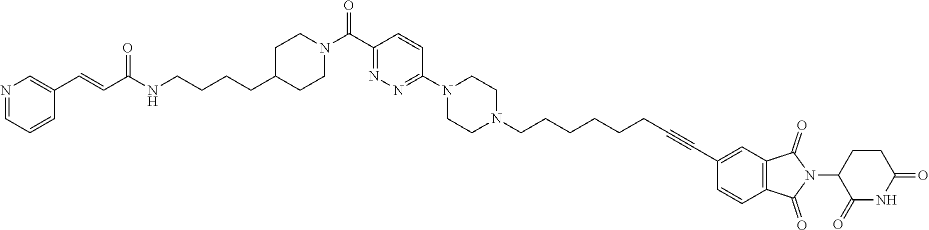 [SMILES: O=C(/C=C/c1cccnc1)NCCCCC1CCN(C(=O)c2ccc(N3CCN(CCCCCCC#Cc4ccc5c(c4)C(=O)N(C4CCC(=O)NC4=O)C5=O)CC3)nn2)CC1] | (E)-N-(4-(1-(6-(4-(8-(2- (2,6-dioxopiperidin-3- yl)-1,3- dioxoisoindolin- 5-yl)oct-7-yn-1- yl)piperazin-1- yl)pyridazine-3- carbonyl)piperidin- 4-yl)butyl)-3- (pyridin-3- yl)acrylamide | (E)-N-(4-(1-(6-(4-(8- (2-(2,6- dioxopiperidin- 3-yl)-1,3- dioxoisoindolin-5- yl)oct-7-yn-1- yl)piperazin-1- yl)pyridazine-3- carbonyl)piperidin- 4-yl)butyl)- 3-(pyridin-3- yl)acrylamide |
| | 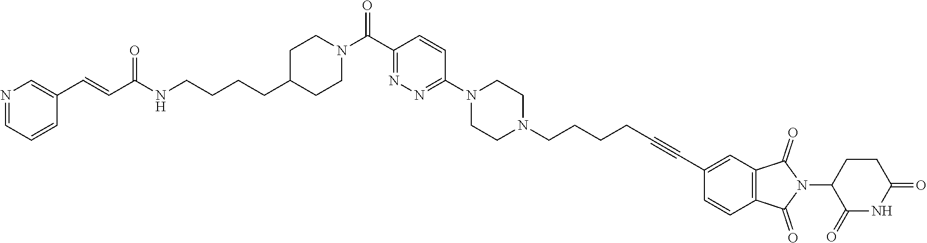 [SMILES: O=C(/C=C/c1cccnc1)NCCCCC1CCN(C(=O)c2ccc(N3CCN(CCCCC#Cc4ccc5c(c4)C(=O)N(C4CCC(=O)NC4=O)C5=O)CC3)nn2)CC1] | (E)-N-(4-(1-(6-(4-(6-(2- (2,6-dioxopiperidin-3- yl)-1,3- dioxoisoindolin- 5-yl)hex-5-yn-1- yl)piperazin-1- yl)pyridazine-3- carbonyl)piperidin-4- yl)butyl)-3-(pyridin- 3-yl)acrylamide | (E)-N-(4-(1-(6-(4-(6- (2-(2,6- dioxopiperidin- 3-yl)-1,3- dioxoisoindolin-5- yl)hex-5-yn-1- yl)piperazin-1- yl)pyridazine-3- carbonyl)piperidin- 4-yl)butyl)- 3-(pyridin-3- yl)acrylamide |
| | 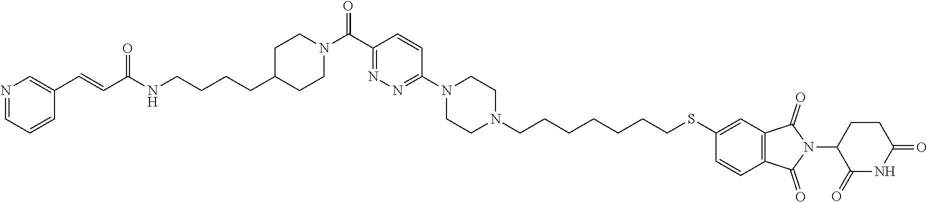 [SMILES: O=C(/C=C/c1cccnc1)NCCCCC1CCN(C(=O)c2ccc(N3CCN(CCCCCCCSc4ccc5c(c4)C(=O)N(C4CCC(=O)NC4=O)C5=O)CC3)nn2)CC1] | (E)-N-(4-(1-(6-(4-(7- ((2-(2,6- dioxopiperidin- 3-yl)-1,3- dioxoisoindolin-5- yl)thio)heptyl) piperazin- 1-yl)pyridazine-3- carbonyl)piperidin-4- yl)butyl)-3-(pyridin- 3-yl)acrylamide | (E)-N-(4-(1-(6-(4-(7- ((2-(2,6- dioxopiperidin-3-yl)- 1,3-dioxoisoindolin- 5-yl)thio)heptyl) piperazin-1-yl) pyridazine-3- carbonyl)piperidin- 4-yl)butyl)- 3-(pyridin-3- yl)acrylamide |
| | 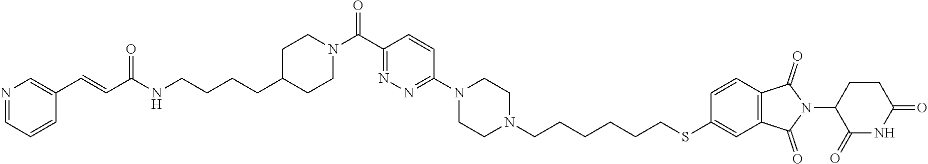 [SMILES: O=C(/C=C/c1cccnc1)NCCCCC1CCN(C(=O)c2ccc(N3CCN(CCCCCCSc4ccc5c(c4)C(=O)N(C4CCC(=O)NC4=O)C5=O)CC3)nn2)CC1] | (E)-N-(4-(1-(6-(4-(6- ((2-(2,6- dioxopiperidin- 3-yl)-1,3- dioxoisoindolin-5- yl)thio)hexyl) piperazin-1-yl) pyridazine-3- carbonyl)piperidin- 4-yl)butyl)- 3-(pyridin-3- yl)acrylamide | (E)-N-(4-(1-(6-(4-(6- ((2-(2,6- dioxopiperidin-3-yl)- 1,3-dioxoisoindolin- 5-yl)thio)hexyl) piperazin-1- yl)pyridazine-3- carbonyl)piperidin- 4-yl)butyl)- 3-(pyridin-3- yl)acrylamide |
| | 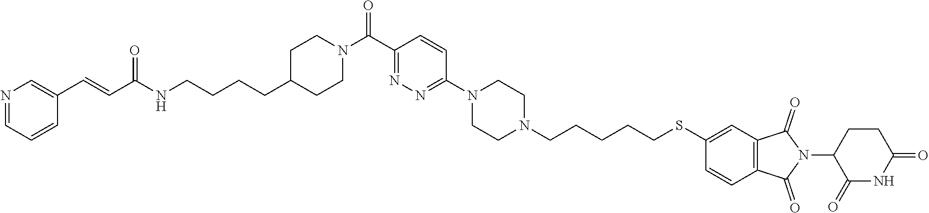 [SMILES: O=C(/C=C/c1cccnc1)NCCCCC1CCN(C(=O)c2ccc(N3CCN(CCCCCSc4ccc5c(c4)C(=O)N(C4CCC(=O)NC4=O)C5=O)CC3)nn2)CC1] | (E)-N-(4-(1-(6-(4-(5- ((2-(2,6- dioxopiperidin- 3-yl)-1,3- dioxoisoindolin-5- yl)thio)pentyl) piperazin-1-yl) pyridazine-3- carbonyl)piperidin-4- yl)butyl)-3-(pyridin- 3-yl)acrylamide | (E)-N-(4-(1-(6-(4-(5- ((2-(2,6- dioxopiperidin-3-yl)- 1,3-dioxoisoindolin- 5-yl)thio)pentyl) piperazin-1- yl)pyridazine-3- carbonyl)piperidin- 4-yl)butyl)- 3-(pyridin-3- yl)acrylamide |
| | 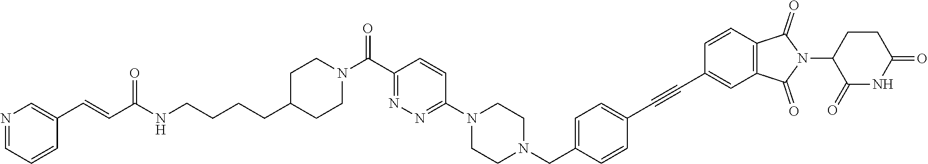 [SMILES: O=C(/C=C/c1cccnc1)NCCCCC1CCN(C(=O)c2ccc(N3CCN(Cc4ccc(C#Cc5ccc6c(c5)C(=O)N(C5CCC(=O)NC5=O)C6=O)cc4)CC3)nn2)CC1] | (E)-N-(4-(1-(6-(4-(4- ((2-(2,6-dioxopiperidin- 3-yl)-1,3- dioxoisoindolin-5- yl)ethynyl)benzyl) piperazin-1- yl)pyridazine-3- carbonyl)piperidin-4- yl)butyl)-3-(pyridin- 3-yl)acrylamide | (E)-N-(4-(1-(6-(4-(4- ((2-(2,6- dioxopiperidin-3-yl)- 1,3-dioxoisoindolin- 5-yl)ethynyl)benzyl) piperazin-1- yl)pyridazine-3- carbonyl)piperidin- 4-yl)butyl)- 3-(pyridin-3- yl)acrylamide |
| | 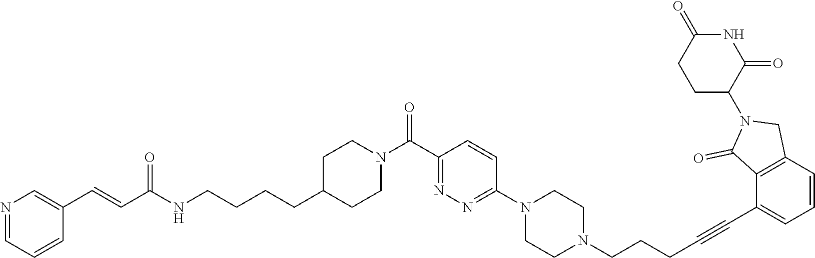 [SMILES: O=C(/C=C/c1cccnc1)NCCCCC1CCN(C(=O)c2ccc(N3CCN(CCCC#Cc4cccc5c4C(=O)N(C4CCC(=O)NC4=O)C5)CC3)nn2)CC1] | (E)-N-(4-(1-(6-(4-(5-(2- (2,6-dioxopiperidin-3- yl)-3-oxoisoindolin-4- yl)pent-4-yn-1- yl)piperazin-1- yl)pyridazine-3- carbonyl)piperidin-4- yl)butyl)-3-(pyridin- 3-yl)acrylamide | (E)-N-(4-(1-(6-(4-(5- (2-(2,6- dioxopiperidin- 3-yl)-3- oxoisoindolin- 4-yl)pent-4-yn-1- yl)piperazin-1- yl)pyridazine-3- carbonyl)piperidin- 4-yl)butyl)- 3-(pyridin-3- yl)acrylamide |
| | 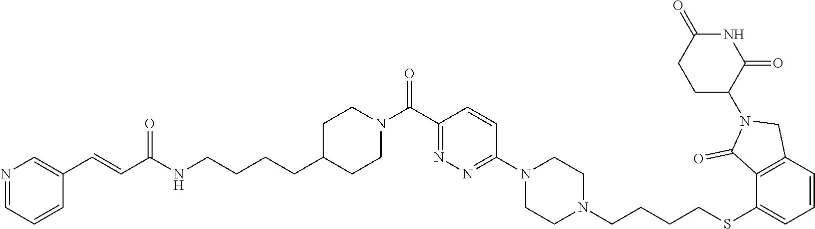 [SMILES: O=C(/C=C/c1cccnc1)NCCCCC1CCN(C(=O)c2ccc(N3CCN(CCCCSc4cccc5c4C(=O)N(C4CCC(=O)NC4=O)C5)CC3)nn2)CC1] | (E)-N-(4-(1-(6-(4-(4- ((2-(2,6- dioxopiperidin- 3-yl)-3- oxoisoindolin-4- yl)thio)butyl) piperazin- 1-yl)pyridazine-3- carbonyl)piperidin- 4-yl)butyl)-3- (pyridin-3- yl)acrylamide | (E)-N-(4-(1-(6-(4-(4- ((2-(2,6- dioxopiperidin-3- yl)-3- oxoisoindolin-4- yl)thio)butyl) piperazin- 1-yl)pyridazine-3- carbonyl)piperidin- 4-yl)butyl)-3- (pyridin-3- yl)acrylamide |
| | 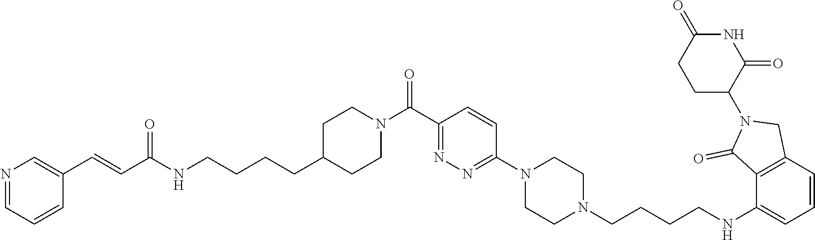 [SMILES: O=C(/C=C/c1cccnc1)NCCCCC1CCN(C(=O)c2ccc(N3CCN(CCCCNc4cccc5c4C(=O)N(C4CCC(=O)NC4=O)C5)CC3)nn2)CC1] | (E)-N-(4-(1-(6-(4-(4- ((2-(2,6- dioxopiperidin- 3-yl)-3- oxoisoindolin-4- yl)amino)butyl) piperazin-1- yl)pyridazine-3- carbonyl)piperidin- 4-yl)butyl)- 3-(pyridin-3- yl)acrylamide | (E)-N-(4-(1-(6-(4-(4- ((2-(2,6- dioxopiperidin-3- yl)-3- oxoisoindolin-4- yl)amino)butyl) piperazin-1- yl)pyridazine-3- carbonyl)piperidin- 4-yl)butyl)-3- (pyridin-3- yl)acrylamide |
| | 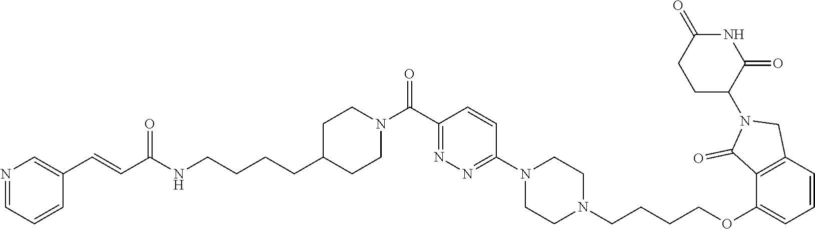 [SMILES: O=C(/C=C/c1cccnc1)NCCCCC1CCN(C(=O)c2ccc(N3CCN(CCCCOc4cccc5c4C(=O)N(C4CCC(=O)NC4=O)C5)CC3)nn2)CC1] | (E)-N-(4-(1-(6-(4-(4- ((2-(2,6- dioxopiperidin- 3-yl)-3- oxoisoindolin-4- yl)oxy)butyl) piperazin-1-yl) pyridazine-3- carbonyl)piperidin- 4-yl)butyl)- 3-(pyridin-3- yl)acrylamide | (E)-N-(4-(1-(6-(4-(4- ((2-(2,6- dioxopiperidin- 3-yl)-3- oxoisoindolin-4- yl)oxy)butyl) piperazin-1- yl)pyridazine-3- carbonyl)piperidin- 4-yl)butyl)-3- (pyridin-3- yl)acrylamide |
| | 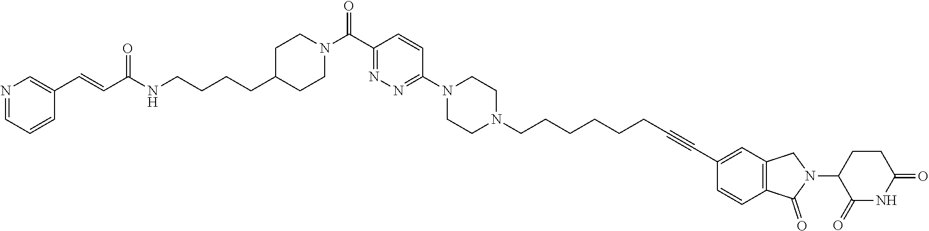 [SMILES: O=C(/C=C/c1cccnc1)NCCCCC1CCN(C(=O)c2ccc(N3CCN(CCCCCCC#Cc4ccc5c(c4)CN(C4CCC(=O)NC4=O)C5=O)CC3)nn2)CC1] | (E)-N-(4-(1-(6-(4-(8-(2- (2,6-dioxopiperidin-3- yl)-1-oxoisoindolin-5- yl)oct-7-yn-1- yl)piperazin-1- yl)pyridazine-3- carbonyl)piperidin-4- yl)butyl)-3-(pyridin-3- yl)acrylamide | (E)-N-(4-(1-(6-(4-(8- (2-(2,6- dioxopiperidin- 3-yl)-1- oxoisoindolin- 5-yl)oct-7-yn-1- yl)piperazin-1- yl)pyridazine-3- carbonyl)piperidin- 4-yl)butyl)- 3-(pyridin-3- yl)acrylamide |
| | 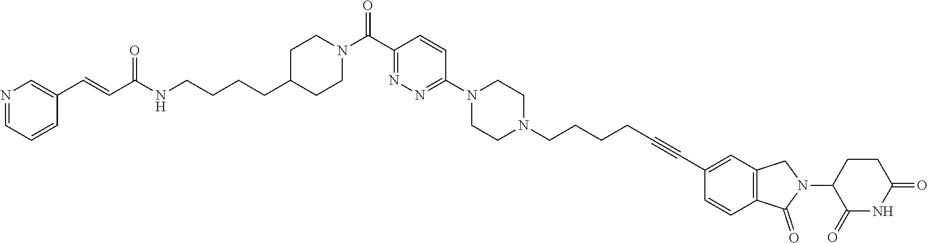 [SMILES: O=C(/C=C/c1cccnc1)NCCCCC1CCN(C(=O)c2ccc(N3CCN(CCCCC#Cc4ccc5c(c4)CN(C4CCC(=O)NC4=O)C5=O)CC3)nn2)CC1] | (E)-N-(4-(1-(6-(4-(6-(2- (2,6-dioxopiperidin-3- yl)-1-oxoisoindolin-5- yl)hex-5-yn-1- yl)piperazin-1- yl)pyridazine-3- carbonyl)piperidin-4- yl)butyl)-3-(pyridin-3- yl)acrylamide | (E)-N-(4-(1-(6-(4-(6- (2-(2,6- dioxopiperidin- 3-yl)-1- oxoisoindolin- 5-yl)hex-5-yn-1- yl)piperazin-1- yl)pyridazine-3- carbonyl)piperidin- 4-yl)butyl)- 3-(pyridin-3- yl)acrylamide |
| | 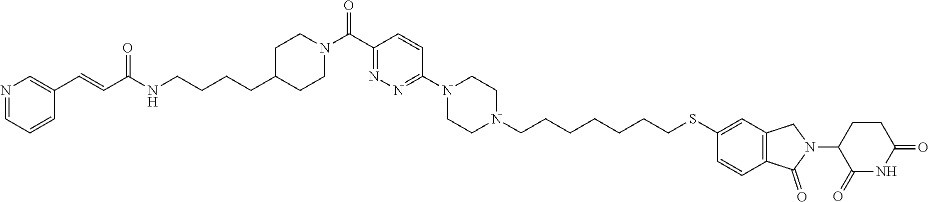 [SMILES: O=C(/C=C/c1cccnc1)NCCCCC1CCN(C(=O)c2ccc(N3CCN(CCCCCCCSc4ccc5c(c4)CN(C4CCC(=O)NC4=O)C5=O)CC3)nn2)CC1] | (E)-N-(4-(1-(6-(4-(7- ((2-(2,6- dioxopiperidin- 3-yl)-1- oxoisoindolin-5- yl)thio)heptyl) piperazin- 1-yl)pyridazine-3- carbonyl)piperidin- 4-yl)butyl)- 3-(pyridin-3- yl)acrylamide | (E)-N-(4-(1-(6-(4-(7- ((2-(2,6- dioxopiperidin- 3-yl)-1- oxoisoindolin-5- yl)thio)heptyl) piperazin-1- yl)pyridazine-3- carbonyl)piperidin- 4-yl)butyl)-3- (pyridin-3- yl)acrylamide |
| | 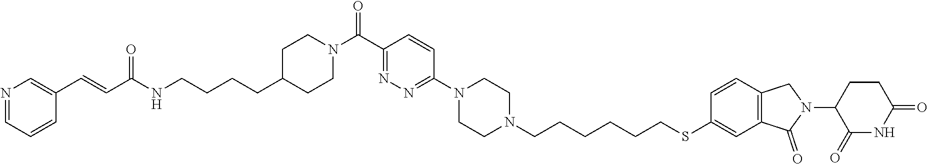 [SMILES: O=C(/C=C/c1cccnc1)NCCCCC1CCN(C(=O)c2ccc(N3CCN(CCCCCCSc4ccc5c(c4)C(=O)N(C4CCC(=O)NC4=O)C5)CC3)nn2)CC1] | (E)-N-(4-(1-(6-(4-(6- ((2-(2,6- dioxopiperidin- 3-yl)-3- oxoisoindolin-5- yl)thio)hexyl) piperazin-1-yl) pyridazine-3- carbonyl)piperidin- 4-yl)butyl)- 3-(pyridin- 3-yl)acrylamide | (E)-N-(4-(1-(6-(4-(6- ((2-(2,6- dioxopiperidin- 3-yl)-3- oxoisoindolin-5- yl)thio)hexyl) piperazin-1- yl)pyridazine-3- carbonyl)piperidin- 4-yl)butyl)- 3-(pyridin-3- yl)acrylamide |
| | 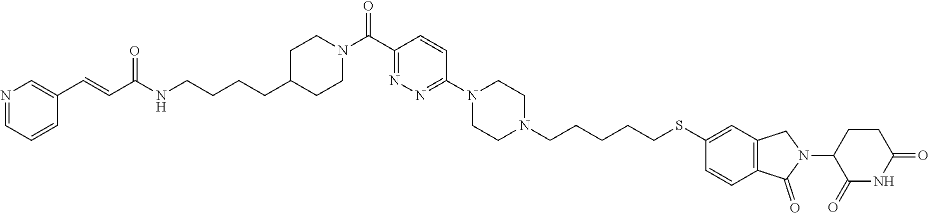 [SMILES: O=C(/C=C/c1cccnc1)NCCCCC1CCN(C(=O)c2ccc(N3CCN(CCCCCSc4ccc5c(c4)CN(C4CCC(=O)NC4=O)C5=O)CC3)nn2)CC1] | (E)-N-(4-(1-(6-(4-(5- ((2-(2,6- dioxopiperidin- 3-yl)-1- oxoisoindolin-5- yl)thio)pentyl) piperazin- 1-yl)pyridazine-3- carbonyl)piperidin- 4-yl)butyl)- 3-(pyridin-3- yl)acrylamide | (E)-N-(4-(1-(6-(4-(5- ((2-(2,6- dioxopiperidin- 3-yl)-1- oxoisoindolin-5- yl)thio)pentyl) piperazin-1- yl)pyridazine-3- carbonyl)piperidin- 4-yl)butyl)- 3-(pyridin-3- yl)acrylamide |
| | 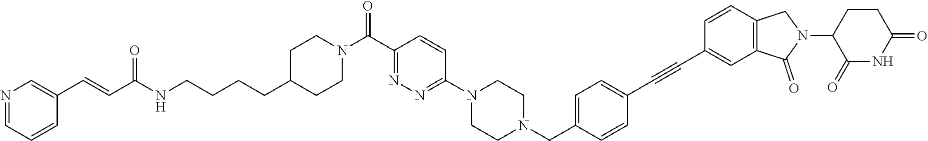 [SMILES: O=C(/C=C/c1cccnc1)NCCCCC1CCN(C(=O)c2ccc(N3CCN(Cc4ccc(C#Cc5ccc6c(c5)C(=O)N(C5CCC(=O)NC5=O)C6)cc4)CC3)nn2)CC1] | (E)-N-(4-(1-(6-(4-(4- ((2-(2,6- dioxopiperidin- 3-yl)-3- oxoisoindolin-5- yl)ethynyl)benzyl) piperazin-1- yl)pyridazine-3- carbonyl)piperidin- 4-yl)butyl)- 3-(pyridin-3- yl)acrylamide | (E)-N-(4-(1-(6-(4-(4- ((2-(2,6- dioxopiperidin- 3-yl)-3- oxoisoindolin-5- yl)ethynyl)benzyl) piperazin-1- yl)pyridazine-3- carbonyl)piperidin- 4-yl)butyl)- 3-(pyridin-3- yl)acrylamide |
| | 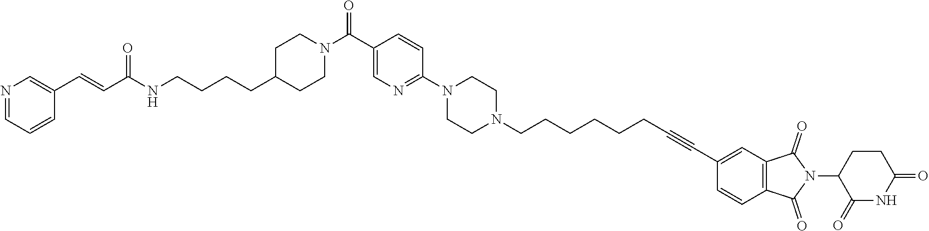 [SMILES: O=C(/C=C/c1cccnc1)NCCCCC1CCN(C(=O)c2ccc(N3CCN(CCCCCCC#Cc4ccc5c(c4)C(=O)N(C4CCC(=O)NC4=O)C5=O)CC3)nc2)CC1] | (E)-N-(4-(1-(6-(4-(8-(2- (2,6-dioxopiperidin-3- yl)-1,3- dioxoisoindolin- 5-yl)oct-7-yn-1- yl)piperazin-1- yl)nicotinoyl) piperidin-4-yl)butyl)- 3-(pyridin-3- yl)acrylamide | (E)-N-(4-(1-(6-(4-(8- (2-(2,6- dioxopiperidin- 3-yl)-1,3- dioxoisoindolin-5- yl)oct-7-yn-1- yl)piperazin-1- yl)nicotinoyl) piperidin-4- yl)butyl)- 3-(pyridin- 3-yl)acrylamide |
| | 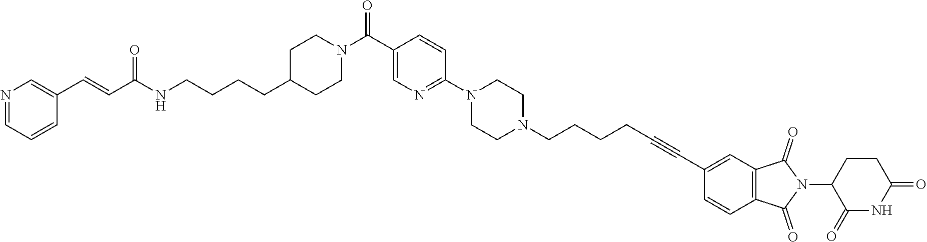 [SMILES: O=C(/C=C/c1cccnc1)NCCCCC1CCN(C(=O)c2ccc(N3CCN(CCCCC#Cc4ccc5c(c4)C(=O)N(C4CCC(=O)NC4=O)C5=O)CC3)nc2)CC1] | (E)-N-(4-(1-(6-(4-(6-(2- (2,6-dioxopiperidin-3- yl)-1,3- dioxoisoindolin- 5-yl)hex-5-yn-1- yl)piperazin-1- yl)nicotinoyl) piperidin-4- yl)butyl)-3- (pyridin-3- yl)acrylamide | (E)-N-(4-(1-(6-(4-(6- (2-(2,6- dioxopiperidin- 3-yl)-1,3- dioxoisoindolin-5- yl)hex-5-yn-1- yl)piperazin-1- yl)nicotinoyl) piperidin-4- yl)butyl)- 3-(pyridin- 3-yl)acrylamide |
| | 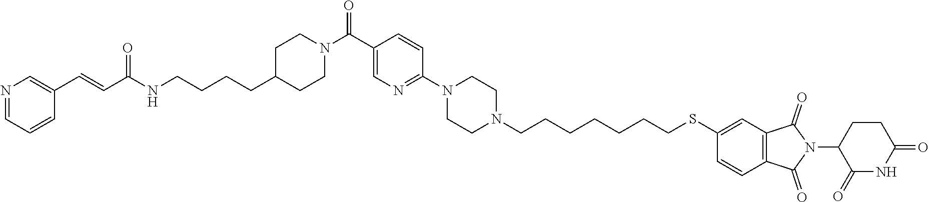 [SMILES: O=C(/C=C/c1cccnc1)NCCCCC1CCN(C(=O)c2ccc(N3CCN(CCCCCCCSc4ccc5c(c4)C(=O)N(C4CCC(=O)NC4=O)C5=O)CC3)nc2)CC1] | (E)-N-(4-(1-(6-(4-(7- ((2-(2,6- dioxopiperidin- 3-yl)-1,3- dioxoisoindolin-5- yl)thio)heptyl) piperazin-1- yl)nicotinoyl) piperidin- 4-yl)butyl)- 3-(pyridin-3- yl)acrylamide | (E)-N-(4-(1-(6-(4-(7- ((2-(2,6- dioxopiperidin- 3-yl)-1,3- dioxoisoindolin-5- yl)thio)heptyl) piperazin-1- yl)nicotinoyl) piperidin- 4-yl)butyl)- 3-(pyridin- 3-yl)acrylamide |
| | 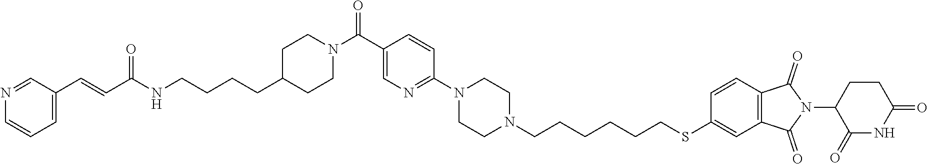 [SMILES: O=C(/C=C/c1cccnc1)NCCCCC1CCN(C(=O)c2ccc(N3CCN(CCCCCCSc4ccc5c(c4)C(=O)N(C4CCC(=O)NC4=O)C5=O)CC3)nc2)CC1] | (E)-N-(4-(1-(6-(4-(6- ((2-(2,6- dioxopiperidin- 3-yl)-1,3- dioxoisoindolin- 5-yl)thio)hexyl) piperazin-1- yl)nicotinoyl) piperidin-4- yl)butyl)-3-(pyridin- 3-yl)acrylamide | (E)-N-(4-(1-(6-(4-(6- ((2-(2,6- dioxopiperidin- 3-yl)-1,3- dioxoisoindolin-5- yl)thio)hexyl) piperazin-1- yl)nicotinoyl) piperidin-4-yl) butyl)-3-(pyridin- 3-yl)acrylamide |
| | 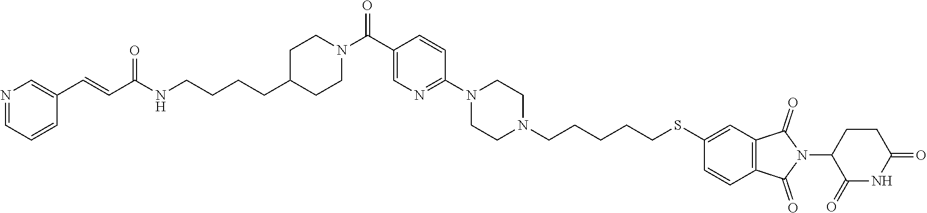 [SMILES: O=C(/C=C/c1cccnc1)NCCCCC1CCN(C(=O)c2ccc(N3CCN(CCCCCSc4ccc5c(c4)C(=O)N(C4CCC(=O)NC4=O)C5=O)CC3)nc2)CC1] | (E)-N-(4-(1-(6-(4-(5- ((2-(2,6- dioxopiperidin- 3-yl)-1,3- dioxoisoindolin-5- yl)thio)pentyl) piperazin-1- yl)nicotinoyl) piperidin- 4-yl)butyl)- 3-(pyridin-3- yl)acrylamide | (E)-N-(4-(1-(6-(4-(5- ((2-(2,6- dioxopiperidin- 3-yl)-1,3- dioxoisoindolin-5- yl)thio)pentyl) piperazin-1- yl)nicotinoyl) piperidin- 4-yl)butyl)- 3-(pyridin- 3-yl)acrylamide |
| | 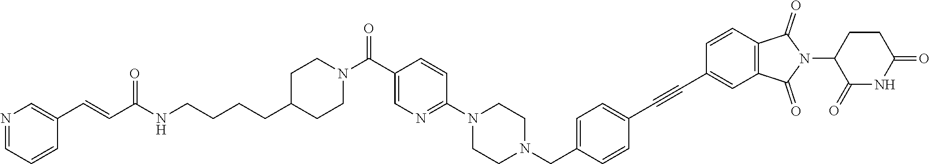 [SMILES: O=C(/C=C/c1cccnc1)NCCCCC1CCN(C(=O)c2ccc(N3CCN(Cc4ccc(C#Cc5ccc6c(c5)C(=O)N(C5CCC(=O)NC5=O)C6=O)cc4)CC3)nc2)CC1] | (E)-N-(4-(1-(6-(4-(4- ((2-(2,6- dioxopiperidin- 3-yl)-1,3- dioxoisoindolin-5- yl)ethynyl)benzyl) piperazin-1- yl)nicotinoyl) piperidin- 4-yl)butyl)- 3-(pyridin-3- yl)acrylamide | (E)-N-(4-(1-(6-(4-(4- ((2-(2,6- dioxopiperidin- 3-yl)-1,3- dioxoisoindolin-5- yl)ethynyl)benzyl) piperazin-1- yl)nicotinoyl) piperidin- 4-yl)butyl)- 3-(pyridin- 3-yl)acrylamide |
| | 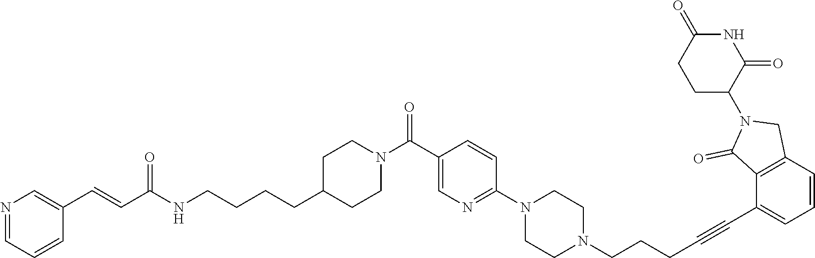 [SMILES: O=C(/C=C/c1cccnc1)NCCCCC1CCN(C(=O)c2ccc(N3CCN(CCCC#Cc4cccc5c4C(=O)N(C4CCC(=O)NC4=O)C5)CC3)nc2)CC1] | (E)-N-(4-(1-(6-(4-(5-(2- (2,6-dioxopiperidin-3- yl)-3-oxoisoindolin-4- yl)pent-4-yn-1- yl)piperazin-1- yl)nicotinoyl) piperidin- 4-yl)butyl)- 3-(pyridin-3- yl)acrylamide | (E)-N-(4-(1-(6-(4-(5- (2-(2,6- dioxopiperidin- 3-yl)-3- oxoisoindolin- 4-yl)pent-4-yn-1- yl)piperazin-1- yl)nicotinoyl) piperidin- 4-yl)butyl)- 3-(pyridin- 3-yl)acrylamide |
| | 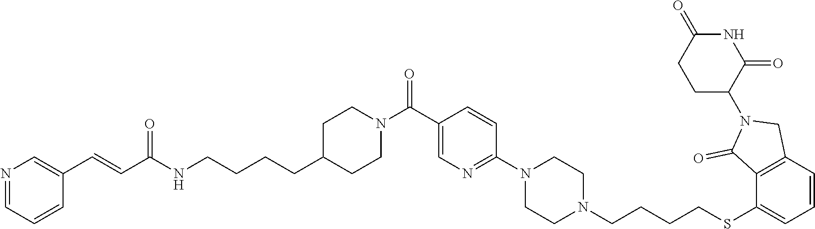 [SMILES: O=C(/C=C/c1cccnc1)NCCCCC1CCN(C(=O)c2ccc(N3CCN(CCCCSc4cccc5c4C(=O)N(C4CCC(=O)NC4=O)C5)CC3)nc2)CC1] | (E)-N-(4-(1-(6-(4-(4- ((2-(2,6- dioxopiperidin- 3-yl)-3- oxoisoindolin-4- yl)thio)butyl) piperazin-1- yl)nicotinoyl) piperidin- 4-yl)butyl)- 3-(pyridin-3- yl)acrylamide | (E)-N-(4-(1-(6-(4-(4- ((2-(2,6- dioxopiperidin- 3-yl)-3- oxoisoindolin-4- yl)thio)butyl) piperazin-1- yl)nicotinoyl) piperidin- 4-yl)butyl)- 3-(pyridin- 3-yl)acrylamide |
| | 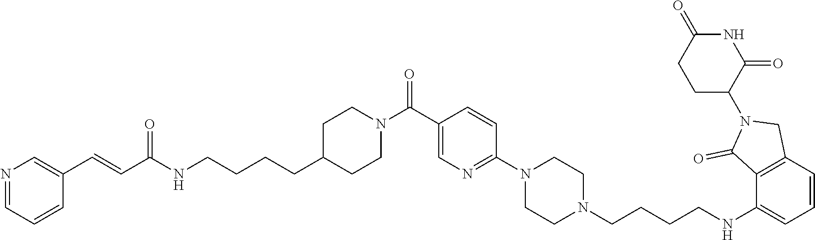 [SMILES: O=C(/C=C/c1cccnc1)NCCCCC1CCN(C(=O)c2ccc(N3CCN(CCCCNc4cccc5c4C(=O)N(C4CCC(=O)NC4=O)C5)CC3)nc2)CC1] | (E)-N-(4-(1-(6-(4-(4- ((2-(2,6- dioxopiperidin- 3-yl)-3- oxoisoindolin-4- yl)amino)butyl) piperazin-1- yl)nicotinoyl) piperidin- 4-yl)butyl)- 3-(pyridin-3- yl)acrylamide | (E)-N-(4-(1-(6-(4-(4- ((2-(2,6- dioxopiperidin- 3-yl)-3- oxoisoindolin-4- yl)amino)butyl) piperazin-1- yl)nicotinoyl) piperidin- 4-yl)butyl)- 3-(pyridin- 3-yl)acrylamide |
| | 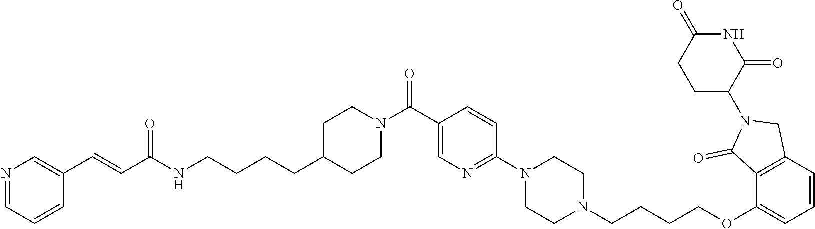 [SMILES: O=C(/C=C/c1cccnc1)NCCCCC1CCN(C(=O)c2ccc(N3CCN(CCCCOc4cccc5c4C(=O)N(C4CCC(=O)NC4=O)C5)CC3)nc2)CC1] | (E)-N-(4-(1-(6-(4-(4- ((2-(2,6- dioxopiperidin- 3-yl)-3- oxoisoindolin-4- yl)oxy)butyl) piperazin-1- yl)nicotinoyl) piperidin- 4-yl)butyl)- 3-(pyridin-3- yl)acrylamide | (E)-N-(4-(1-(6-(4-(4- ((2-(2,6- dioxopiperidin- 3-yl)-3- oxoisoindolin-4- yl)oxy)butyl) piperazin-1- yl)nicotinoyl) piperidin- 4-yl)butyl)- 3-(pyridin- 3-yl)acrylamide |
| | 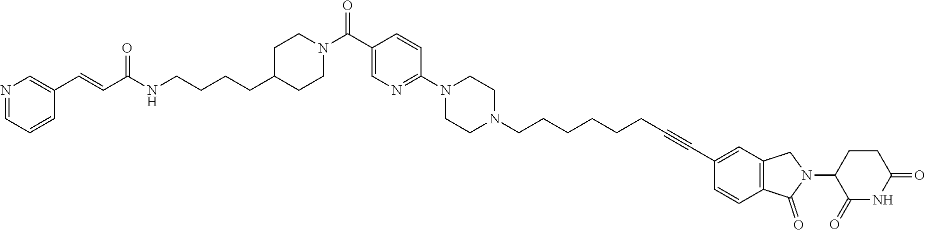 [SMILES: O=C(/C=C/c1cccnc1)NCCCCC1CCN(C(=O)c2ccc(N3CCN(CCCCCCC#Cc4ccc5c(c4)CN(C4CCC(=O)NC4=O)C5=O)CC3)nc2)CC1] | (E)-N-(4-(1-(6-(4-(8-(2- (2,6-dioxopiperidin-3- yl)-1-oxoisoindolin-5- yl)oct-7-yn-1- yl)piperazin-1 - yl)nicotinoyl) piperidin- 4-yl)butyl)- 3-(pyridin-3- yl)acrylamide | (E)-N-(4-(1-(6-(4-(8- (2-(2,6- dioxopiperidin- 3-yl)-1- oxoisoindolin- 5-yl)oct-7-yn-1- yl)piperazin-1- yl)nicotinoyl) piperidin- 4-yl)butyl)- 3-(pyridin- 3-yl)acrylamide |
| | 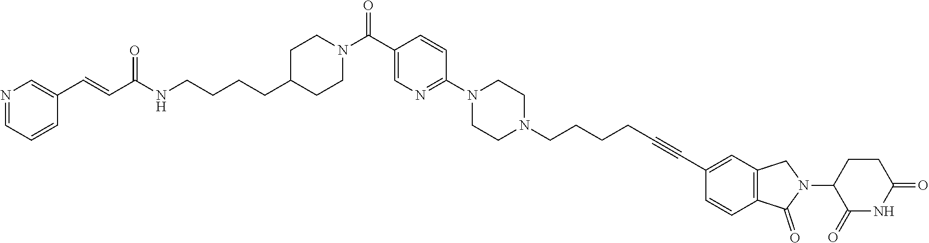 [SMILES: O=C(/C=C/c1cccnc1)NCCCCC1CCN(C(=O)c2ccc(N3CCN(CCCCC#Cc4ccc5c(c4)CN(C4CCC(=O)NC4=O)C5=O)CC3)nc2)CC1] | (E)-N-(4-(1-(6-(4-(6-(2- (2,6-dioxopiperidin-3- yl)-1-oxoisoindolin-5- yl)hex-5-yn-1- yl)piperazin-1- yl)nicotinoyl) piperidin- 4-yl)butyl)- 3-(pyridin-3- yl)acrylamide | (E)-N-(4-(1-(6-(4-(6- (2-(2,6- dioxopiperidin- 3-yl)-1- oxoisoindolin- 5-yl)hex-5-yn-1- yl)piperazin-1- yl)nicotinoyl) piperidin- 4-yl)butyl)- 3-(pyridin- 3-yl)acrylamide |
| | 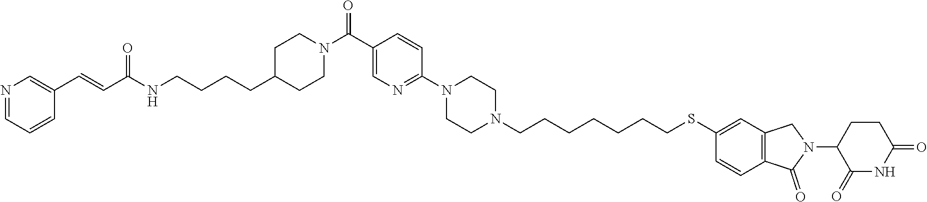 [SMILES: O=C(/C=C/c1cccnc1)NCCCCC1CCN(C(=O)c2ccc(N3CCN(CCCCCCCSc4ccc5c(c4)CN(C4CCC(=O)NC4=O)C5=O)CC3)nc2)CC1] | (E)-N-(4-(1-(6-(4-(7- ((2-(2,6- dioxopiperidin- 3-yl)-1- oxoisoindolin-5- yl)thio)heptyl) piperazin-1- yl)nicotinoyl) piperidin- 4-yl)butyl)- 3-(pyridin-3- yl)acrylamide | (E)-N-(4-(1-(6-(4-(7- ((2-(2,6- dioxopiperidin- 3-yl)-1- oxoisoindolin-5- yl)thio)heptyl) piperazin-1- yl)nicotinoyl) piperidin- 4-yl)butyl)- 3-(pyridin- 3-yl)acrylamide |
| | 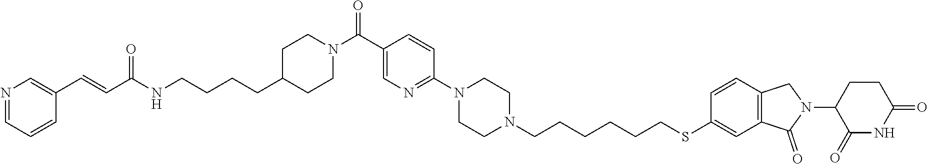 [SMILES: O=C(/C=C/c1cccnc1)NCCCCC1CCN(C(=O)c2ccc(N3CCN(CCCCCCSc4ccc5c(c4)C(=O)N(C4CCC(=O)NC4=O)C5)CC3)nc2)CC1] | (E)-N-(4-(1-(6-(4-(6- ((2-(2,6- dioxopiperidin- 3-yl)-3- oxoisoindolin-5- yl)thio)hexyl) piperazin-1- yl)nicotinoyl) piperidin- 4-yl)butyl)- 3-(pyridin-3- yl)acrylamide | (E)-N-(4-(1-(6-(4-(6- ((2-(2,6- dioxopiperidin- 3-yl)-3- oxoisoindolin-5- yl)thio)hexyl) piperazin-1- yl)nicotinoyl) piperidin- 4-yl)butyl)- 3-(pyridin- 3-yl)acrylamide |
| | 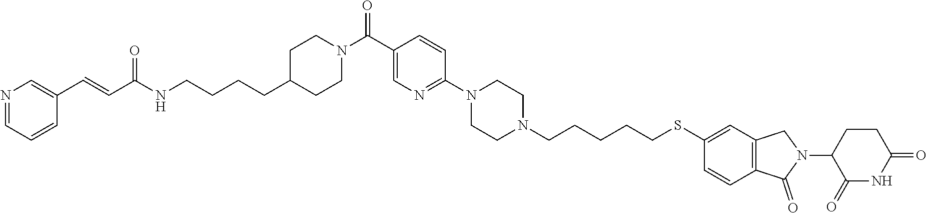 [SMILES: O=C(/C=C/c1cccnc1)NCCCCC1CCN(C(=O)c2ccc(N3CCN(CCCCCSc4ccc5c(c4)CN(C4CCC(=O)NC4=O)C5=O)CC3)nc2)CC1] | (E)-N-(4-(1-(6-(4-(5- ((2-(2,6- dioxopiperidin- 3-yl)-1- oxoisoindolin-5- yl)thio)pentyl) piperazin-1- yl)nicotinoyl) piperidin- 4-yl)butyl)- 3-(pyridin-3- yl)acrylamid | (E)-N-(4-(1-(6-(4-(5- ((2-(2,6- dioxopiperidin- 3-yl)-1- oxoisoindolin-5- yl)thio)pentyl) piperazin-1- yl)nicotinoyl) piperidin- 4-yl)butyl)- 3-(pyridin- 3-yl)acrylamide |
| | 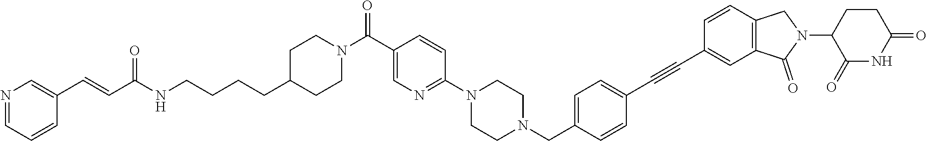 [SMILES: O=C(/C=C/c1cccnc1)NCCCCC1CCN(C(=O)c2ccc(N3CCN(Cc4ccc(C#Cc5ccc6c(c5)C(=O)N(C5CCC(=O)NC5=O)C6)cc4)CC3)nc2)CC1] | (E)-N-(4-(1-(6-(4-(4- ((2-(2,6- dioxopiperidin- 3-yl)-3- oxoisoindolin-5- yl)ethynyl)benzyl) piperazin-1- yl)nicotinoyl) piperidin- 4-yl)butyl)- 3-(pyridin-3- yl)acrylamide | (E)-N-(4-(1-(6-(4-(4- ((2-(2,6- dioxopiperidin- 3-yl)-3- oxoisoindolin-5- yl)ethynyl)benzyl) piperazin-1- yl)nicotinoyl) piperidin- 4-yl)butyl)- 3-(pyridin- 3-yl)acrylamide |
| | 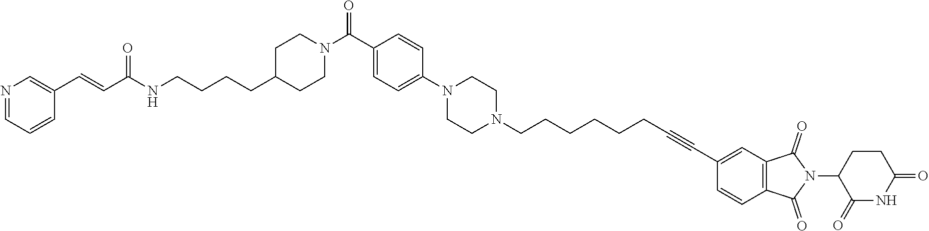 [SMILES: O=C(/C=C/c1cccnc1)NCCCCC1CCN(C(=O)c2ccc(N3CCN(CCCCCCC#Cc4ccc5c(c4)C(=O)N(C4CCC(=O)NC4=O)C5=O)CC3)cc2)CC1] | (E)-N-(4-(1-(4-(4-(8-(2- (2,6-dioxopiperidin-3- yl)-1,3- dioxoisoindolin- 5-yl)oct-7-yn-1- yl)piperazin-1- yl)benzoyl)piperidin- 4-yl)butyl)- 3-(pyridin-3- yl)acrylamide | (E)-N-(4-(1-(4-(4-(8- (2-(2,6- dioxopiperidin- 3-yl)-1,3- dioxoisoindolin-5- yl)oct-7-yn-1- yl)piperazin-1- yl)benzoyl) piperidin-4- yl)butyl)-3- (pyridin-3- yl)acrylamide |
| | 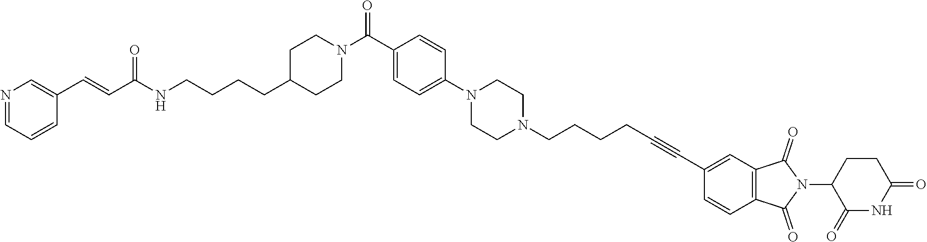 [SMILES: O=C(/C=C/c1cccnc1)NCCCCC1CCN(C(=O)c2ccc(N3CCN(CCCCC#Cc4ccc5c(c4)C(=O)N(C4CCC(=O)NC4=O)C5=O)CC3)cc2)CC1] | (E)-N-(4-(1-(4-(4-(6-(2- (2,6-dioxopiperidin-3- yl)-1,3- dioxoisoindolin- 5-yl)hex-5-yn-1- yl)piperazin-1- yl)benzoyl)piperidin- 4-yl)butyl)- 3-(pyridin-3- yl)acrylamide | (E)-N-(4-(1-(4-(4-(6- (2-(2,6- dioxopiperidin- 3-yl)-1,3- dioxoisoindolin-5- yl)hex-5-yn-1- yl)piperazin-1- yl)benzoyl) piperidin-4- yl)butyl)-3- (pyridin-3- yl)acrylamide |
| | 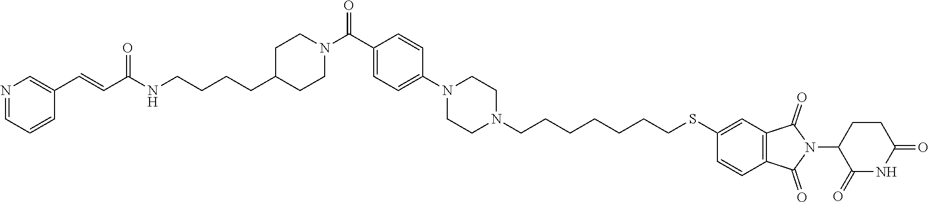 [SMILES: O=C(/C=C/c1cccnc1)NCCCCC1CCN(C(=O)c2ccc(N3CCN(CCCCCCCSc4ccc5c(c4)C(=O)N(C4CCC(=O)NC4=O)C5=O)CC3)cc2)CC1] | (E)-N-(4-(1-(4-(4-(7- ((2-(2,6-d ioxopiperidin- 3-yl)-1,3- dioxoisoindolin-5- yl)thio)heptyl) piperazin- 1-yl)benzoyl) piperidin- 4-yl)butyl)-3- (pyridin-3- yl)acrylamide | (E)-N-(4-(1-(4-(4-(7- ((2-(2,6- dioxopiperidin- 3-yl)-1,3- dioxoisoindolin-5- yl)thio)heptyl) piperazin-1- yl)benzoyl) piperidin-4- yl)butyl)-3- (pyridin-3- yl)acrylamide |
| | 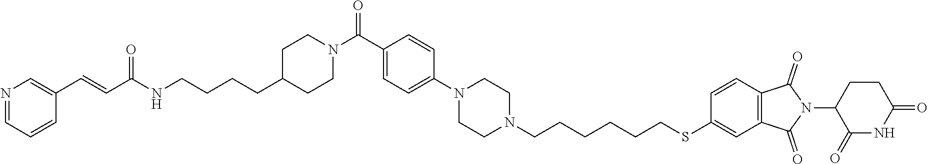 [SMILES: O=C(/C=C/c1cccnc1)NCCCCC1CCN(C(=O)c2ccc(N3CCN(CCCCCCSc4ccc5c(c4)C(=O)N(C4CCC(=O)NC4=O)C5=O)CC3)cc2)CC1] | (E)-N-(4-(1-(4-(4-(6- ((2-(2,6- dioxopiperidin- 3-yl)-1,3- dioxoisoindolin-5- yl)thio)hexyl) piperazin- 1-yl)benzoyl) piperidin- 4-yl)butyl)-3- (pyridin-3- yl)acrylamide | (E)-N-(4-(1-(4-(4-(6- ((2-(2,6- dioxopiperidin- 3-yl)-1,3- dioxoisoindolin-5- yl)thio)hexyl) piperazin-1- yl)benzoyl) piperidin-4- yl)butyl)-3- (pyridin-3- yl)acrylamide |
| | 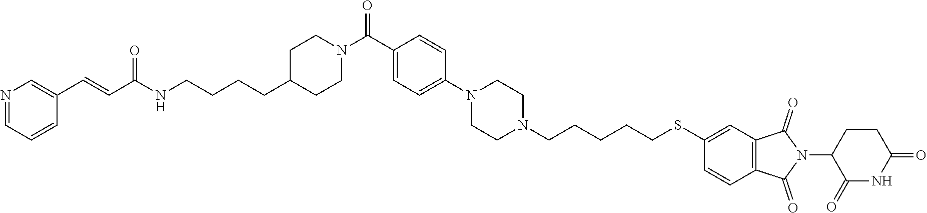 [SMILES: O=C(/C=C/c1cccnc1)NCCCCC1CCN(C(=O)c2ccc(N3CCN(CCCCCSc4ccc5c(c4)C(=O)N(C4CCC(=O)NC4=O)C5=O)CC3)cc2)CC1] | (E)-N-(4-(1-(4-(4-(5- ((2-(2,6- dioxopiperidin- 3-yl)-1,3- dioxoisoindolin-5- yl)thio)pentyl) piperazin- 1-yl)benzoyl) piperidin- 4-yl)butyl)-3- (pyridin-3- yl)acrylamide | (E)-N-(4-(1-(4-(4-(5- ((2-(2,6- dioxopiperidin- 3-yl)-1,3- dioxoisoindolin- 5-yl)thio)pentyl) piperazin-1- yl)benzoyl) piperidin-4- yl)butyl)-3- (pyridin-3- yl)acrylamide |
| | 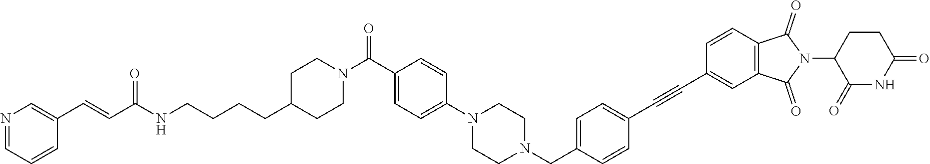 [SMILES: O=C(/C=C/c1cccnc1)NCCCCC1CCN(C(=O)c2ccc(N3CCN(Cc4ccc(C#Cc5ccc6c(c5)C(=O)N(C5CCC(=O)NC5=O)C6=O)cc4)CC3)cc2)CC1] | (E)-N-(4-(1-(4-(4-(4- ((2-(2,6- dioxopiperidin- 3-yl)-1,3- dioxoisoindolin-5- yl)ethynyl)benzyl) piperazin-1- yl)benzoyl)piperidin- 4-yl)butyl)- 3-(pyridin-3- yl)acrylamide | (E)-N-(4-(1-(4-(4-(4- ((2-(2,6- dioxopiperidin- 3-yl)-1,3- dioxoisoindolin-5- yl)ethynyl)benzyl) piperazin-1- yl)benzoyl)piperidin- 4-yl)butyl)- 3-(pyridin-3- yl)acrylamide |
| | 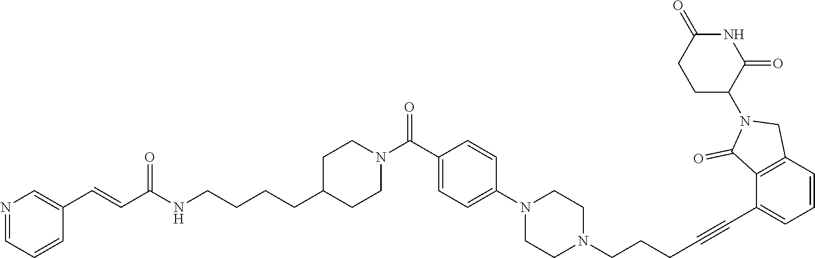 [SMILES: O=C(/C=C/c1cccnc1)NCCCCC1CCN(C(=O)c2ccc(N3CCN(CCCC#Cc4cccc5c4C(=O)N(C4CCC(=O)NC4=O)C5)CC3)cc2)CC1] | (E)-N-(4-(1-(4-(4-(5-(2- (2,6-dioxopiperidin-3- yl)-3-oxoisoindolin-4- yl)pent-4-yn-1- yl)piperazin-1- yl)benzoyl)piperidin- 4-yl)butyl)- 3-(pyridin-3- yl)acrylamide | (E)-N-(4-(1-(4-(4-(5- (2-(2,6- dioxopiperidin- 3-yl)-3- oxoisoindolin- 4-yl)pent-4-yn-1- yl)piperazin-1- yl)benzoyl)piperidin- 4-yl)butyl)- 3-(pyridin-3- yl)acrylamide |
| | 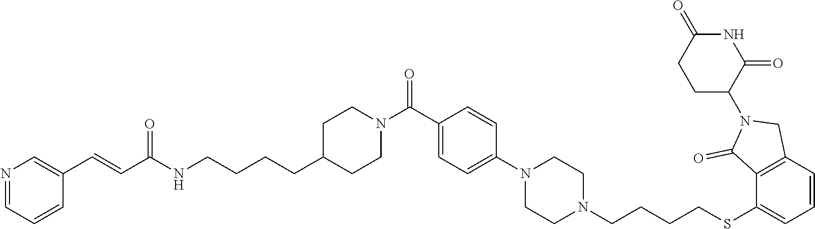 [SMILES: O=C(/C=C/c1cccnc1)NCCCCC1CCN(C(=O)c2ccc(N3CCN(CCCCSc4cccc5c4C(=O)N(C4CCC(=O)NC4=O)C5)CC3)cc2)CC1] | (E)-N-(4-(1-(4-(4-(4- ((2-(2,6- dioxopiperidin- 3-yl)-3- oxoisoindolin-4- yl)thio)butyl) piperazin- 1-yl)benzoyl) piperidin- 4-yl)butyl)- 3-(pyridin-3- yl)acrylamide | (E)-N-(4-(1-(4-(4-(4- ((2-(2,6- dioxopiperidin- 3-yl)-3- oxoisoindolin-4- yl)thio)butyl) piperazin- 1-yl)benzoyl) piperidin- 4-yl)butyl)- 3-(pyridin- 3-yl)acrylamide |
| | 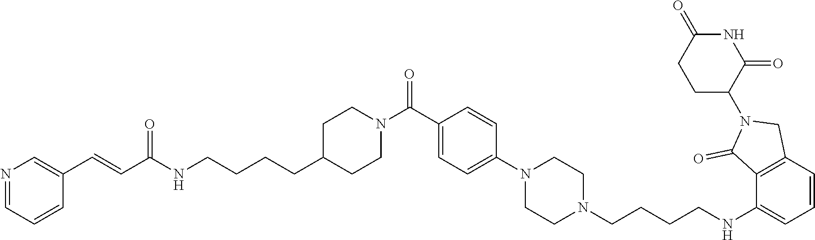 [SMILES: O=C(/C=C/c1cccnc1)NCCCCC1CCN(C(=O)c2ccc(N3CCN(CCCCNc4cccc5c4C(=O)N(C4CCC(=O)NC4=O)C5)CC3)cc2)CC1] | (E)-N-(4-(1-(4-(4-(4- ((2-(2,6- dioxopiperidin- 3-yl)-3- oxoisoindolin-4- yl)amino)butyl) piperazin-1- yl)benzoyl) piperidin-4- yl)butyl)- 3-(pyridin-3- yl)acrylamide | (E)-N-(4-(1-(4-(4-(4- ((2-(2,6- dioxopiperidin- 3-yl)-3- oxoisoindolin-4- yl)amino)butyl) piperazin-1- yl)benzoyl) piperidin-4- yl)butyl)-3- (pyridin-3- yl)acrylamide |
| | 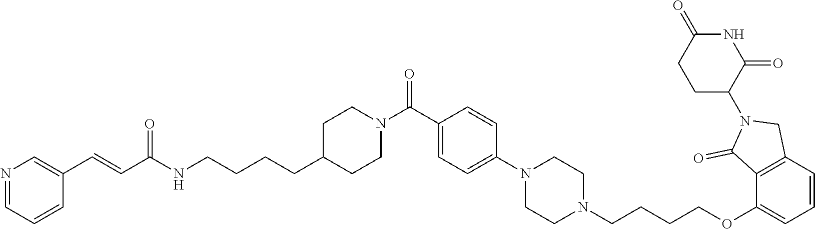 [SMILES: O=C(/C=C/c1cccnc1)NCCCCC1CCN(C(=O)c2ccc(N3CCN(CCCCOc4cccc5c4C(=O)N(C4CCC(=O)NC4=O)C5)CC3)cc2)CC1] | (E)-N-(4-(1-(4-(4-(4- ((2-(2,6- dioxopiperidin- 3-yl)-3- oxoisoindolin-4- yl)oxy)butyl) piperazin- 1-yl)benzoyl) piperidin- 4-yl)butyl)- 3-(pyridin-3- yl)acrylamide | (E)-N-(4-(1-(4-(4-(4- ((2-(2,6- dioxopiperidin- 3-yl)-3- oxoisoindolin-4- yl)oxy)butyl) piperazin- 1-yl)benzoyl) piperidin- 4-yl)butyl)- 3-(pyridin- 3-yl)acrylamide |
| | 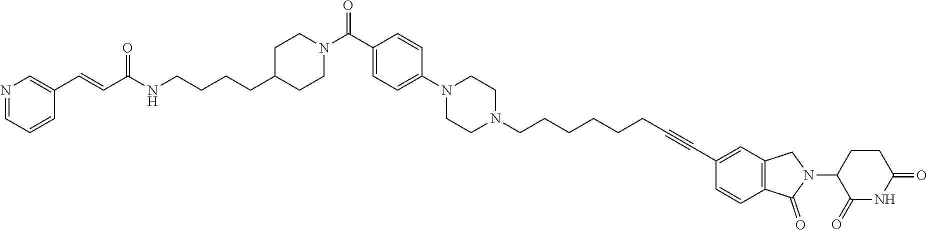 [SMILES: O=C(/C=C/c1cccnc1)NCCCCC1CCN(C(=O)c2ccc(N3CCN(CCCCCCC#Cc4ccc5c(c4)CN(C4CCC(=O)NC4=O)C5=O)CC3)cc2)CC1] | (E)-N-(4-(1-(4-(4-(8-(2- (2,6- dioxopiperidin-3- yl)-1-oxoisoindolin-5- yl)oct-7-yn-1- yl)piperazin-1- yl)benzoyl)piperidin- 4-yl)butyl)- 3-(pyridin-3- yl)acrylamide | (E)-N-(4-(1-(4-(4-(8- (2-(2,6- dioxopiperidin- 3-yl)-1- oxoisoindolin- 5-yl)oct-7-yn-1- yl)piperazin-1- yl)benzoyl) piperidin-4- yl)butyl)- 3-(pyridin-3- yl)acrylamide |
| | 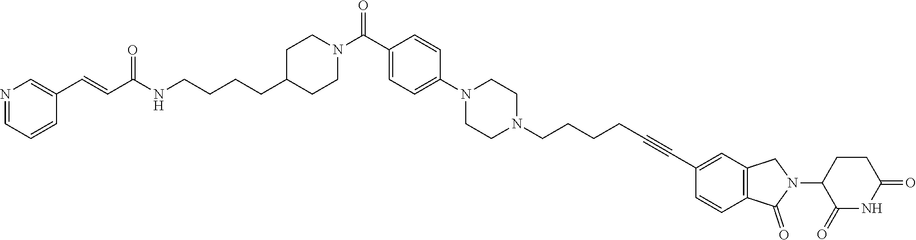 [SMILES: O=C(/C=C/c1cccnc1)NCCCCC1CCN(C(=O)c2ccc(N3CCN(CCCCC#Cc4ccc5c(c4)CN(C4CCC(=O)NC4=O)C5=O)CC3)cc2)CC1] | (E)-N-(4-(1-(4-(4-(6-(2- (2,6-dioxopiperidin-3- yl)-1-oxoisoindolin-5- yl)hex-5-yn-1- yl)piperazin-1- yl)benzoyl)piperidin- 4-yl)butyl)- 3-(pyridin-3- yl)acrylamide | (E)-N-(4-(1-(4-(4-(6- (2-(2,6- dioxopiperidin- 3-yl)-1-o xoisoindolin- 5-yl)hex-5-yn-1- yl)piperazin-1- yl)benzoyl)piperidin- 4-yl)butyl)- 3-(pyridin-3- yl)acrylamide |
| | 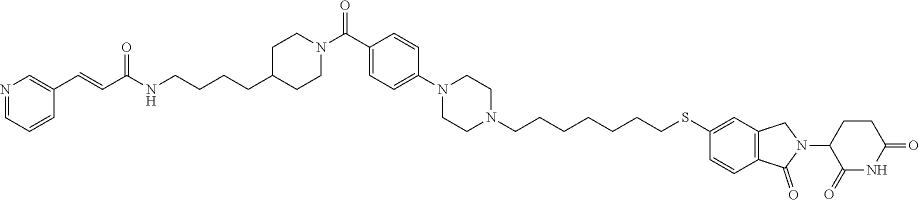 [SMILES: O=C(/C=C/c1cccnc1)NCCCCC1CCN(C(=O)c2ccc(N3CCN(CCCCCCCSc4ccc5c(c4)CN(C4CCC(=O)NC4=O)C5=O)CC3)cc2)CC1] | (E)-N-(4-(1-(4-(4-(7- ((2-(2,6- dioxopiperidin- 3-yl)-1- oxoisoindolin-5- yl)thio)heptyl) piperazin- 1-yl)benzoyl) piperidin- 4-yl)butyl)- 3-(pyridin-3- yl)acrylamide | (E)-N-(4-(1-(4-(4-(7- ((2-(2,6- dioxopiperidin- 3-yl)-1- oxoisoindolin-5- yl)thio)heptyl) piperazin-1- yl)benzoyl) piperidin-4- yl)butyl)-3- (pyridin-3- yl)acrylamide |
| | 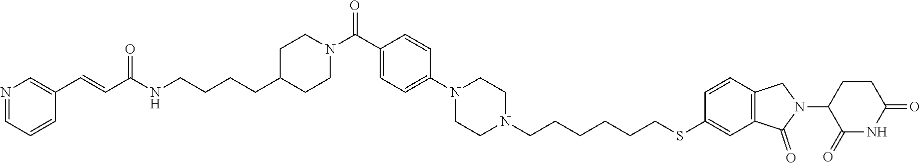 [SMILES: O=C(/C=C/c1cccnc1)NCCCCC1CCN(C(=O)c2ccc(N3CCN(CCCCCCSc4ccc5c(c4)C(=O)N(C4CCC(=O)NC4=O)C5)CC3)cc2)CC1] | (E)-N-(4-(1-(4-(4-(6- ((2-(2,6- dioxopiperidin- 3-yl)-3- oxoisoindolin-5- yl)thio)hexyl) piperazin- 1-yl)benzoyl) piperidin- 4-yl)butyl)- 3-(pyridin-3- yl)acrylamide | (E)-N-(4-(1-(4-(4-(6- ((2-(2,6- dioxopiperidin- 3-yl)-3- oxoisoindolin-5- yl)thio)hexyl) piperazin-1- yl)benzoyl) piperidin-4- yl)butyl)- 3-(pyridin-3- yl)acrylamide |
| | 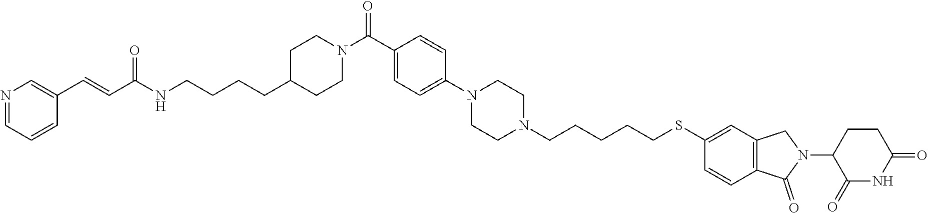 [SMILES: O=C(/C=C/c1cccnc1)NCCCCC1CCN(C(=O)c2ccc(N3CCN(CCCCCSc4ccc5c(c4)CN(C4CCC(=O)NC4=O)C5=O)CC3)cc2)CC1] | (E)-N-(4-(1-(4-(4-(5- ((2-(2,6- dioxopiperidin- 3-yl)-1- oxoisoindolin-5- yl)thio)pentyl) piperazin- 1-yl)benzoyl) piperidin- 4-yl)butyl)- 3-(pyridin-3- yl)acrylamide | (E)-N-(4-(1-(4-(4-(5- ((2-(2,6- dioxopiperidin- 3-yl)-1- oxoisoindolin-5- yl)thio)pentyl) piperazin-1- yl)benzoyl) piperidin-4- yl)butyl)-3- (pyridin-3- yl)acrylamide |
| | 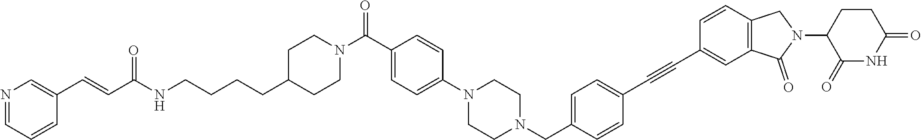 [SMILES: O=C(/C=C/c1cccnc1)NCCCCC1CCN(C(=O)c2ccc(N3CCN(Cc4ccc(C#Cc5ccc6c(c5)C(=O)N(C5CCC(=O)NC5=O)C6)cc4)CC3)cc2)CC1] | (E)-N-(4-(1-(4-(4-(4- ((2-(2,6- dioxopiperidin- 3-yl)-3- oxoisoindolin-5- yl)ethynyl)benzyl) piperazin-1- yl)benzoyl) piperidin-4- yl)butyl)-3-(pyridin- 3-yl)acrylamide | (E)-N-(4-(1-(4-(4-(4- ((2-(2,6- dioxopiperidin- 3-yl)-3- oxoisoindolin-5- yl)ethynyl) benzyl)piperazin-1- yl)benzoyl) piperidin-4- yl)butyl)-3-(pyridin- 3-yl)acrylamide |
| | 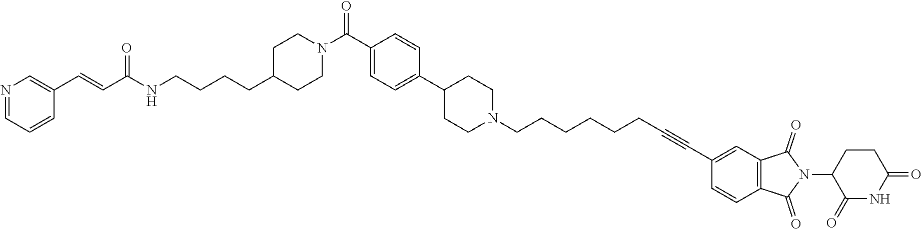 [SMILES: O=C(/C=C/c1cccnc1)NCCCCC1CCN(C(=O)c2ccc(C3CCN(CCCCCCC#Cc4ccc5c(c4)C(=O)N(C4CCC(=O)NC4=O)C5=O)CC3)cc2)CC1] | (E)-N-(4-(1-(4-(1-(8-(2- (2,6-dioxopiperidin-3- yl)-1,3- dioxoisoindolin- 5-yl)oct-7-yn-1- yl)piperidin-4- yl)benzoyl)piperidin- 4-yl)butyl)- 3-(pyridin-3- yl)acrylamide | (E)-N-(4-(1-(4-(1-(8- (2-(2,6- dioxopiperidin- 3-yl)-1,3- dioxoisoindolin-5- yl)oct-7-yn-1- yl)piperidin-4- yl)benzoyl) piperidin-4- yl)butyl)- 3-(pyridin-3- yl)acrylamide |
| | 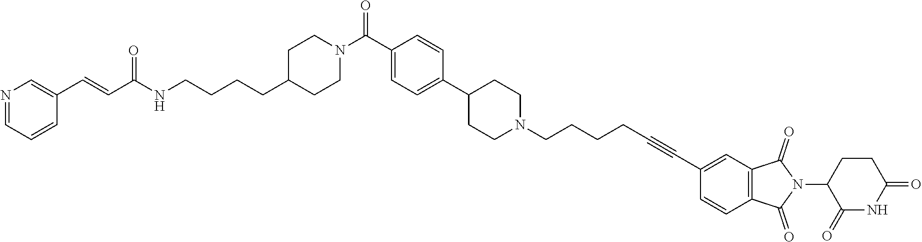 [SMILES: O=C(/C=C/c1cccnc1)NCCCCC1CCN(C(=O)c2ccc(C3CCN(CCCCC#Cc4ccc5c(c4)C(=O)N(C4CCC(=O)NC4=O)C5=O)CC3)cc2)CC1] | (E)-N-(4-(1-(4-(1-(6-(2- (2,6-dioxopiperidin-3- yl)-1,3- dioxoisoindolin- 5-yl)hex-5-yn-1- yl)piperidin-4- yl)benzoyl)piperidin- 4-yl)butyl)- 3-(pyridin-3- yl)acrylamide | (E)-N-(4-(1-(4-(1-(6- (2-(2,6- dioxopiperidin- 3-yl)-1,3- dioxoisoindolin-5- yl)hex-5-yn-1- yl)piperidin-4- yl)benzoyl)piperidin- 4-yl)butyl)- 3-(pyridin-3- yl)acrylamide |
| | 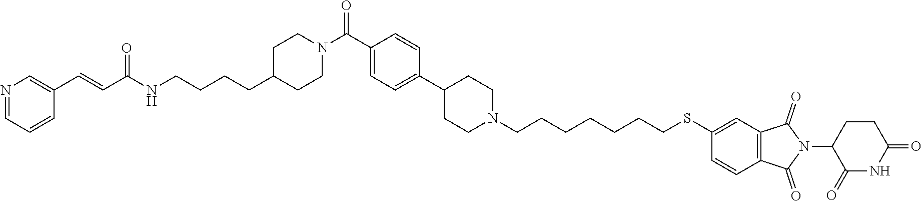 [SMILES: O=C(/C=C/c1cccnc1)NCCCCC1CCN(C(=O)c2ccc(C3CCN(CCCCCCCSc4ccc5c(c4)C(=O)N(C4CCC(=O)NC4=O)C5=O)CC3)cc2)CC1] | (E)-N-(4-(1-(4-(1-(7- ((2-(2,6- dioxopiperidin- 3-yl)-1,3- dioxoisoindolin-5- yl)thio)heptyl) piperidin- 4-yl)benzoyl) piperidin- 4-yl)butyl)- 3-(pyridin-3- yl)acrylamide | (E)-N-(4-(1-(4-(1-(7- ((2-(2,6- dioxopiperidin-3-yl)- 1,3-dioxoisoindolin- 5-yl)thio)heptyl) piperidin-4- yl)benzoyl) piperidin-4- yl)butyl)-3-(pyridin- 3-yl)acrylamide |
| | 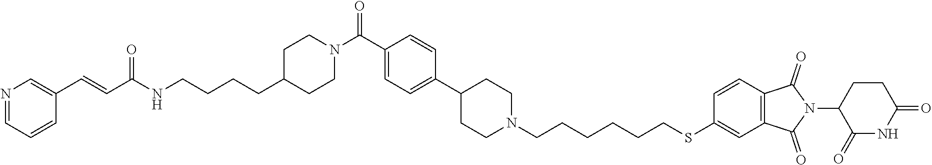 [SMILES: O=C(/C=C/c1cccnc1)NCCCCC1CCN(C(=O)c2ccc(C3CCN(CCCCCCSc4ccc5c(c4)C(=O)N(C4CCC(=O)NC4=O)C5=O)CC3)cc2)CC1] | (E)-N-(4-(1-(4-(1-(6- ((2-(2,6- dioxopiperidin- 3-yl)-1,3- dioxoisoindolin-5- yl)thio)hexyl) piperidin-4-yl) benzoyl)piperidin- 4-yl)butyl)- 3-(pyridin-3- yl)acrylamide | (E)-N-(4-(1-(4-(1-(6- ((2-(2,6- dioxopiperidin- 3-yl)-1,3- dioxoisoindolin-5- yl)thio)hexyl) piperidin-4-yl) benzoyl)piperidin- 4-yl)butyl)- 3-(pyridin- 3-yl)acrylamide |
| | 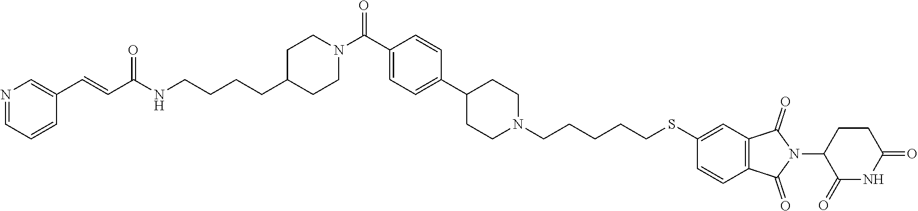 [SMILES: O=C(/C=C/c1cccnc1)NCCCCC1CCN(C(=O)c2ccc(C3CCN(CCCCCSc4ccc5c(c4)C(=O)N(C4CCC(=O)NC4=O)C5=O)CC3)cc2)CC1] | (E)-N-(4-(1-(4-(1-(5- ((2-(2,6- dioxopiperidin- 3-yl)-1,3- dioxoisoindolin-5- yl)thio)pentyl) piperidin-4-yl) benzoyl)piperidin- 4-yl)butyl)-3- (pyridin-3- yl)acrylamide | (E)-N-(4-(1-(4-(1-(5- ((2-(2,6- dioxopiperidin- 3-yl)-1,3- dioxoisoindolin-5- yl)thio)pentyl) piperidin-4-yl) benzoyl)piperidin- 4-yl)butyl)- 3-(pyridin-3- yl)acrylamide |
| | 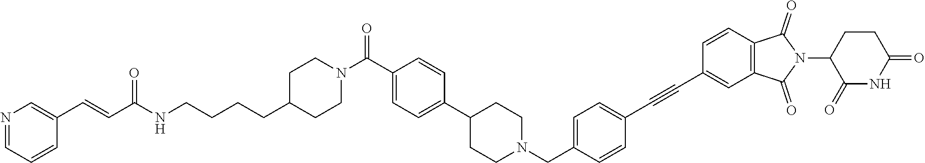 [SMILES: O=C(/C=C/c1cccnc1)NCCCCC1CCN(C(=O)c2ccc(C3CCN(Cc4ccc(C#Cc5ccc6c(c5)C(=O)N(C5CCC(=O)NC5=O)C6=O)cc4)CC3)cc2)CC1] | (E)-N-(4-(1-(4-(1-(4- ((2-(2,6- dioxopiperidin- 3-yl)-1,3- dioxoisoindolin-5- yl)ethynyl)benzyl) piperidin-4- yl)benzoyl) piperidin-4- yl)butyl)-3-(pyridin- 3-yl)acrylamide | (E)-N-(4-(1-(4-(1-(4- ((2-(2,6- dioxopiperidin- 3-yl)-1,3- dioxoisoindolin-5- yl)ethynyl)benzyl) piperidin-4- yl)benzoyl) piperidin-4- yl)butyl)-3-(pyridin- 3-yl)acrylamide |
| | 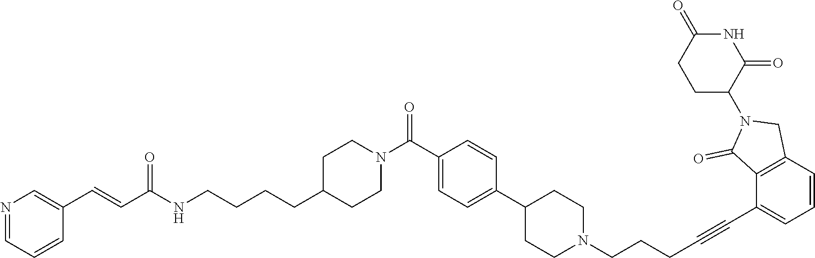 [SMILES: O=C(/C=C/c1cccnc1)NCCCCC1CCN(C(=O)c2ccc(C3CCN(CCCC#Cc4cccc5c4C(=O)N(C4CCC(=O)NC4=O)C5)CC3)cc2)CC1] | (E)-N-(4-(1-(4-(1-(5-(2- (2,6-dioxopiperidin-3- yl)-3-oxoisoindolin-4- yl)pent-4-yn-1- yl)piperidin-4- yl)benzoyl)piperidin- 4-yl)butyl)- 3-(pyridin-3- yl)acrylamide | (E)-N-(4-(1-(4-(1-(5- (2-(2,6- dioxopiperidin- 3-yl)-3- oxoisoindolin- 4-yl)pent-4-yn-1- yl)piperidin-4- yl)benzoyl) piperidin-4- yl)butyl)-3-(pyridin- 3-yl)acrylamide |
| | 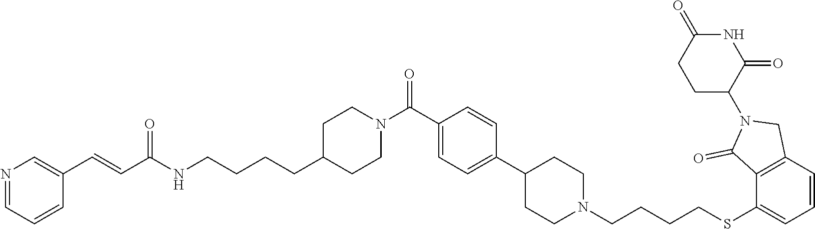 [SMILES: O=C(/C=C/c1cccnc1)NCCCCC1CCN(C(=O)c2ccc(C3CCN(CCCCSc4cccc5c4C(=O)N(C4CCC(=O)NC4=O)C5)CC3)cc2)CC1] | (E)-N-(4-(1-(4-(1-(4- ((2-(2,6- dioxopiperidin- 3-yl)-3- oxoisoindolin-4- yl)thio)butyl) piperidin-4-yl) benzoyl)piperidin- 4-yl)butyl)- 3-(pyridin-3- yl)acrylamide | (E)-N-(4-(1-(4-(1-(4- ((2-(2,6- dioxopiperidin- 3-yl)-3- oxoisoindolin-4- yl)thio)butyl) piperidin-4-yl) benzoyl)piperidin- 4-yl)butyl)-3- (pyridin- 3-yl)acrylamide |
| | 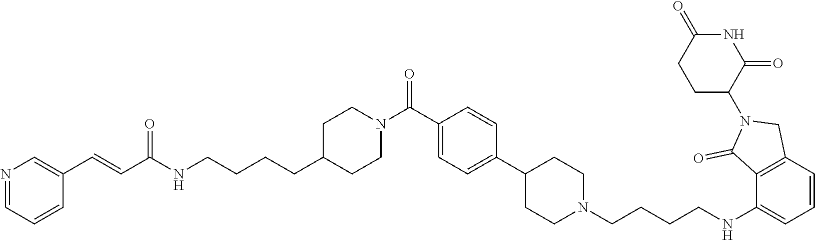 [SMILES: O=C(/C=C/c1cccnc1)NCCCCC1CCN(C(=O)c2ccc(C3CCN(CCCCNc4cccc5c4C(=O)N(C4CCC(=O)NC4=O)C5)CC3)cc2)CC1] | (E)-N-(4-(1-(4-(1-(4- ((2-(2,6- dioxopiperidin- 3-yl)-3- oxoisoindolin-4- yl)amino)butyl) piperidin-4-yl) benzoyl)piperidin- 4-yl)butyl)-3- (pyridin-3- yl)acrylamide | (E)-N-(4-(1-(4-(1-(4- ((2-(2,6- dioxopiperidin- 3-yl)-3- oxoisoindolin-4- yl)amino)butyl) piperidin-4- yl)benzoyl) piperidin-4- yl)butyl)- 3-(pyridin-3- yl)acrylamide |
| | 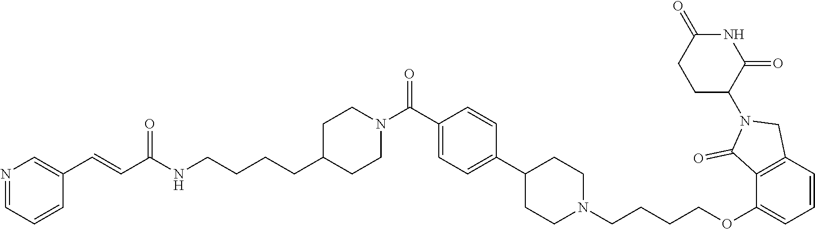 [SMILES: O=C(/C=C/c1cccnc1)NCCCCC1CCN(C(=O)c2ccc(C3CCN(CCCCOc4cccc5c4C(=O)N(C4CCC(=O)NC4=O)C5)CC3)cc2)CC1] | (E)-N-(4-(1-(4-(1-(4- ((2-(2,6- dioxopiperidin- 3-yl)-3- oxoisoindolin-4- yl)oxy)butyl) piperidin-4-yl) benzoyl)piperidin- 4-yl)butyl)- 3-(pyridin-3- yl)acrylamide | (E)-N-(4-(1-(4-(1-(4- ((2-(2,6- dioxopiperidin- 3-yl)-3- oxoisoindolin-4- yl)oxy)butyl) piperidin-4-yl) benzoyl)piperidin- 4-yl)butyl)- 3-(pyridin- 3-yl)acrylamide |
| | 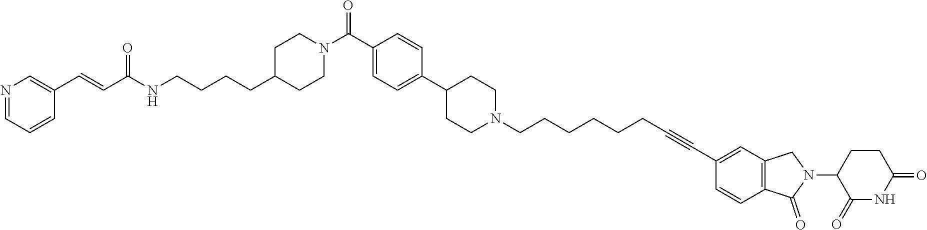 [SMILES: O=C(/C=C/c1cccnc1)NCCCCC1CCN(C(=O)c2ccc(C3CCN(CCCCCCC#Cc4ccc5c(c4)CN(C4CCC(=O)NC4=O)C5=O)CC3)cc2)CC1] | (E)-N-(4-(1-(4-(1-(8-(2- (2,6-dioxopiperidin-3- yl)-1-oxoisoindolin-5- yl)oct-7-yn-1- yl)piperidin-4- yl)benzoyl)piperidin- 4-yl)butyl)- 3-(pyridin-3- yl)acrylamide | (E)-N-(4-(1-(4-(1-(8- (2-(2,6- dioxopiperidin- 3-yl)-1- oxoisoindolin- 5-yl)oct-7-yn-1- yl)piperidin-4- yl)benzoyl) piperidin-4- yl)butyl)- 3-(pyridin-3- yl)acrylamide |
| | 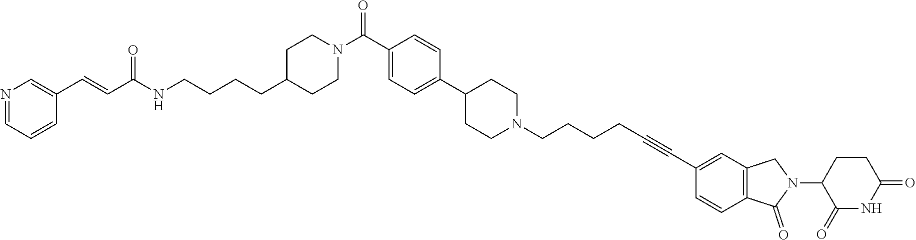 [SMILES: O=C(/C=C/c1cccnc1)NCCCCC1CCN(C(=O)c2ccc(C3CCN(CCCCC#Cc4ccc5c(c4)CN(C4CCC(=O)NC4=O)C5=O)CC3)cc2)CC1] | (E)-N-(4-(1-(4-(1-(6-(2- (2,6-dioxopiperidin-3- yl)-1-oxoisoindolin-5- yl)hex-5-yn-1- yl)piperidin-4- yl)benzoyl)piperidin- 4-yl)butyl)- 3-(pyridin-3- yl)acrylamide | (E)-N-(4-(1-(4-(1-(6- (2-(2,6- dioxopiperidin- 3-yl)-1- oxoisoindolin- 5-yl)hex-5-yn-1- yl)piperidin-4- yl)benzoyl) piperidin-4- yl)butyl)- 3-(pyridin-3- yl)acrylamide |
| | 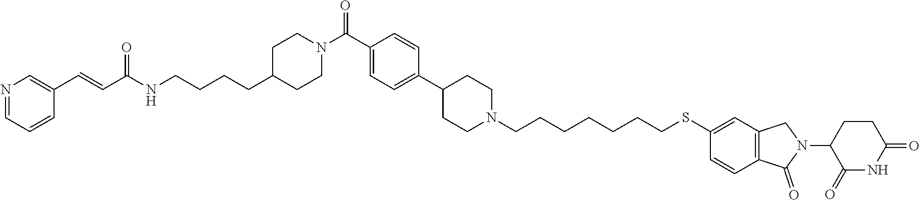 [SMILES: O=C(/C=C/c1cccnc1)NCCCCC1CCN(C(=O)c2ccc(C3CCN(CCCCCCCSc4ccc5c(c4)CN(C4CCC(=O)NC4=O)C5=O)CC3)cc2)CC1] | (E)-N-(4-(1-(4-(1-(7- ((2-(2,6- dioxopiperidin- 3-yl)-1- oxoisoindolin-5- yl)thio)heptyl) piperidin- 4-yl)benzoyl) piperidin- 4-yl)butyl)- 3-(pyridin-3- yl)acrylamide | (E)-N-(4-(1-(4-(1-(7- ((2-(2,6- dioxopiperidin- 3-yl)-1- oxoisoindolin-5- yl)thio)heptyl) piperidin-4- yl)benzoyl) piperidin-4- yl)butyl)- 3-(pyridin-3- yl)acrylamide |
| | 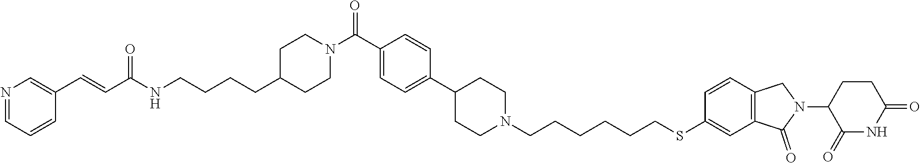 [SMILES: O=C(/C=C/c1cccnc1)NCCCCC1CCN(C(=O)c2ccc(C3CCN(CCCCCCSc4ccc5c(c4)C(=O)N(C4CCC(=O)NC4=O)C5)CC3)cc2)CC1] | (E)-N-(4-(1-(4-(1-(6- ((2-(2,6- dioxopiperidin- 3-yl)-3- oxoisoindolin-5- yl)thio)hexyl) piperidin-4-yl) benzoyl)piperidin- 4-yl)butyl)- 3-(pyridin-3- yl)acrylamide | (E)-N-(4-(1-(4-(1-(6- ((2-(2,6- dioxopiperidin- 3-yl)-3- oxoisoindolin-5- yl)thio)hexyl) piperidin-4-yl) benzoyl)piperidin- 4-yl)butyl)- 3-(pyridin- 3-yl)acrylamide |
| | 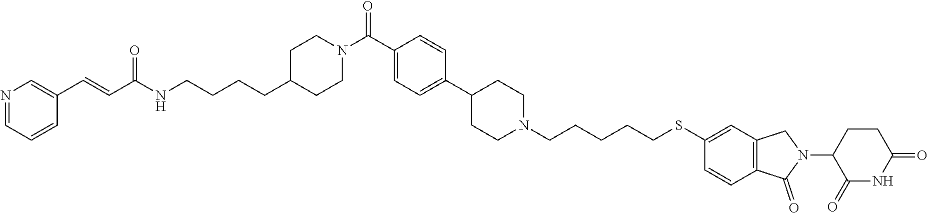 [SMILES: O=C(/C=C/c1cccnc1)NCCCCC1CCN(C(=O)c2ccc(C3CCN(CCCCCSc4ccc5c(c4)CN(C4CCC(=O)NC4=O)C5=O)CC3)cc2)CC1] | (E)-N-(4-(1-(4-(1-(5- ((2-(2,6- dioxopiperidin- 3-yl)-1- oxoisoindolin-5- yl)thio)pentyl) piperidin-4-yl) benzoyl)piperidin- 4-yl)butyl)- 3-(pyridin-3- yl)acrylamide | (E)-N-(4-(1-(4-(1-(5- ((2-(2,6- dioxopiperidin- 3-yl)-1- oxoisoindolin-5- yl)thio)pentyl) piperidin-4- yl)benzoyl) piperidin-4- yl)butyl)- 3-(pyridin-3- yl)acrylamide |
| | 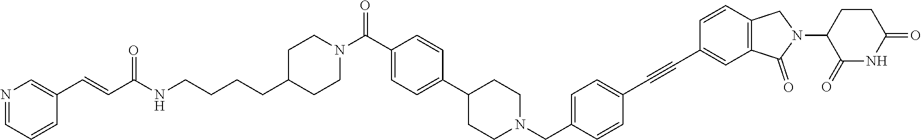 [SMILES: O=C(/C=C/c1cccnc1)NCCCCC1CCN(C(=O)c2ccc(C3CCN(Cc4ccc(C#Cc5ccc6c(c5)C(=O)N(C5CCC(=O)NC5=O)C6)cc4)CC3)cc2)CC1] | (E)-N-(4-(1-(4-(1-(4- ((2-(2,6- dioxopiperidin- 3-yl)-3-oxoisoindolin- 5-yl)ethynyl) benzyl)piperidin-4- yl)benzoyl) piperidin-4- yl)butyl)-3-(pyridin- 3-yl)acrylamide | (E)-N-(4-(1-(4-(1-(4- ((2-(2,6- dioxopiperidin- 3-yl)-3- oxoisoindolin-5- yl)ethynyl)benzyl) piperidin-4- yl)benzoyl)piperidin- 4-yl)butyl)-3- (pyridin-3- yl)acrylamide |
| | 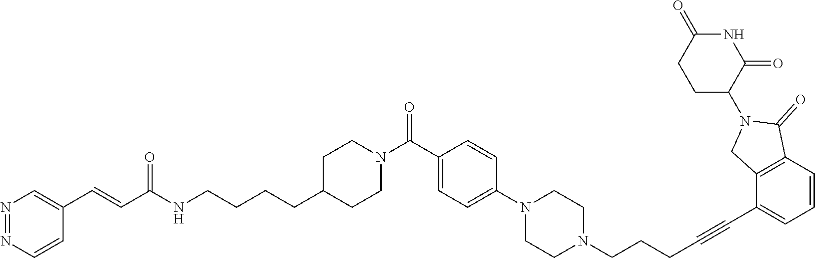 [SMILES: O=C(/C=C/c1ccnnc1)NCCCCC1CCN(C(=O)c2ccc(N3CCN(CCCC#Cc4cccc5c4CN(C4CCC(=O)NC4=O)C5=O)CC3)cc2)CC1] | (E)-N-(4-(1-(4-(4-(7-(2- (2,6-dioxopiperidin-3- yl)-1-oxoisoindolin-4- yl)hept-6-yn-1- yl)piperazin-1- yl)benzoyl)piperidin- 4-yl)butyl)- 3-(pyridazin-4- yl)acrylamide | (E)-N-(4-(1-(4-(4-(7- (2-(2,6- dioxopiperidin- 3-yl)-1- oxoisoindolin- 4-yl)hept-6-yn-1- yl)piperazin-1- yl)benzoyl)piperidin- 4-yl)butyl)- 3-(pyridazin- 4-yl)acrylamide |
| | 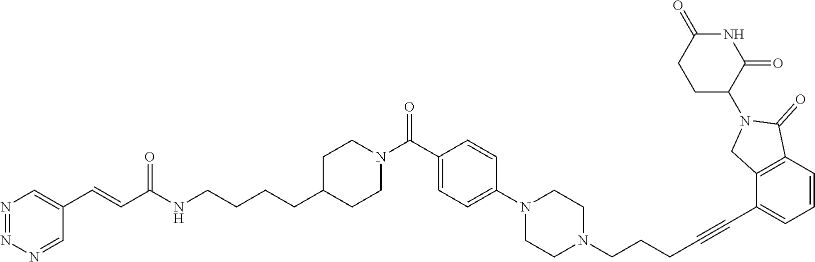 [SMILES: O=C(/C=C/c1cnnnc1)NCCCCC1CCN(C(=O)c2ccc(N3CCN(CCCC#Cc4cccc5c4CN(C4CCC(=O)NC4=O)C5=O)CC3)cc2)CC1] | (E)-N-(4-(1-(4-(4-(7-(2- (2,6-dioxopiperidin-3- yl)-1-oxoisoindolin-4- yl)hept-6-yn-1- yl)piperazin-1- yl)benzoyl)piperidin- 4-yl)butyl)-3-(1,2,3- triazin-5- yl)acrylamide | (E)-N-(4-(1-(4-(4-(7- (2-(2,6- dioxopiperidin- 3-yl)-1- oxoisoindolin- 4-yl)hept-6-yn-1- yl)piperazin-1- yl)benzoyl)piperidin- 4-yl)butyl)-3-(1,2,3- triazin-5- yl)acrylamide |
| | 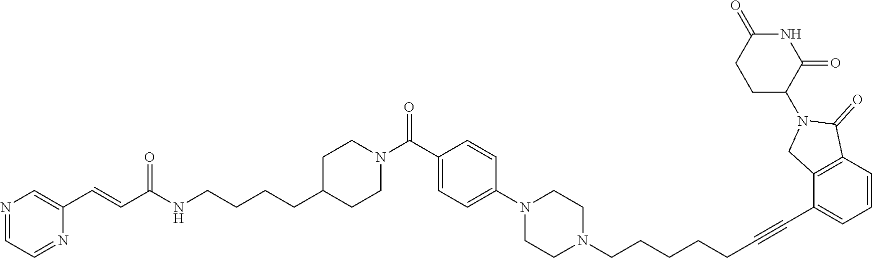 [SMILES: O=C(/C=C/c1cnccn1)NCCCCC1CCN(C(=O)c2ccc(N3CCN(CCCCCC#Cc4cccc5c4CN(C4CCC(=O)NC4=O)C5=O)CC3)cc2)CC1] | (E)-N-(4-(1-(4-(4-(7-(2- (2,6-dioxopiperidin-3- yl)-1-oxoisoindolin-4- yl)hept-6-yn-1- yl)piperazin-1- yl)benzoyl)piperidin- 4-yl)butyl)- 3-(pyrazin-2- yl)acrylamide | (E)-N-(4-(1-(4-(4-(7- (2-(2,6- dioxopiperidin- 3-yl)-1- oxoisoindolin- 4-yl)hept-6-yn-1- yl)piperazin-1- yl)benzoyl) piperidin-4- yl)butyl)- 3-(pyrazin-2- yl)acrylamide |
| | 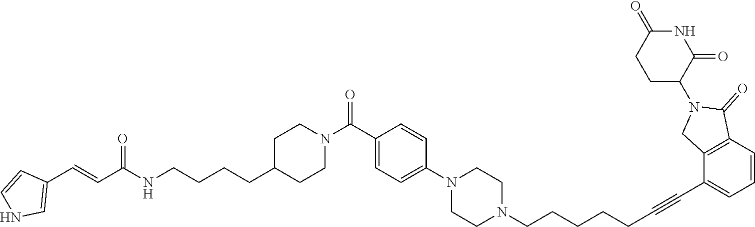 [SMILES: O=C(/C=C/c1cc[nH]c1)NCCCCC1CCN(C(=O)c2ccc(N3CCN(CCCCCC#Cc4cccc5c4CN(C4CCC(=O)NC4=O)C5=O)CC3)cc2)CC1] | (E)-N-(4-(1-(4-(4-(7-(2- (2,6-dioxopiperidin-3- yl)-1-oxoisoindolin-4- yl)hept-6-yn-1- yl)piperazin-1- yl)benzoyl)piperidin- 4-yl)butyl)- 3-(1H-pyrrol- 3-yl)acrylamide | (E)-N-(4-(1-(4-(4-(7- (2-(2,6- dioxopiperidin- 3-yl)-1- oxoisoindolin- 4-yl)hept-6-yn-1- yl)piperazin-1- yl)benzoyl) piperidin-4- yl)butyl)- 3-(1H-pyrrol- 3-yl)acrylamide |
| | 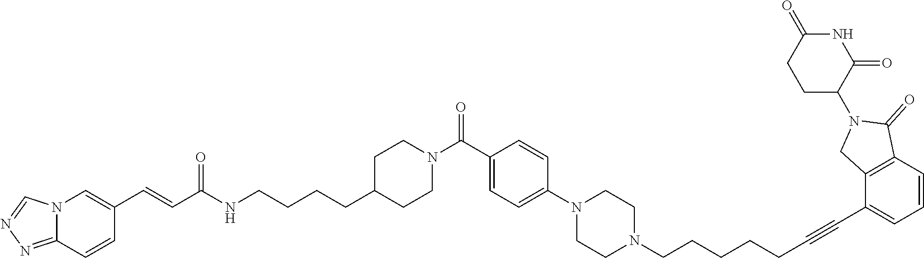 [SMILES: O=C(/C=C/c1ccc2nncn2c1)NCCCCC1CCN(C(=O)c2ccc(N3CCN(CCCCCC#Cc4cccc5c4CN(C4CCC(=O)NC4=O)C5=O)CC3)cc2)CC1] | (E)-3- ([1,2,4]triazolo[4,3- alpyridin-6-yl)-N-(4- (1-(4-(4-(7-(2-(2,6- dioxopiperidin-3- yl)-1-oxoisoindolin- 4-yl)hept-6-yn-1- yl)piperazin-1- yl)benzoyl)piperidin- 4-yl)butyl) acrylamide | (E)-3- ([1,2,4]triazolo[4,3- alpyridin-6-yl)-N-(4- (1-(4-(4-(7-(2-(2,6- dioxopiperidin-3- yl)-1 - oxoisoindolin-4- yl)hept-6-yn-1- yl)piperazin-1- yl)benzoyl) piperidin-4- yl)butyl)acrylamide |
| | 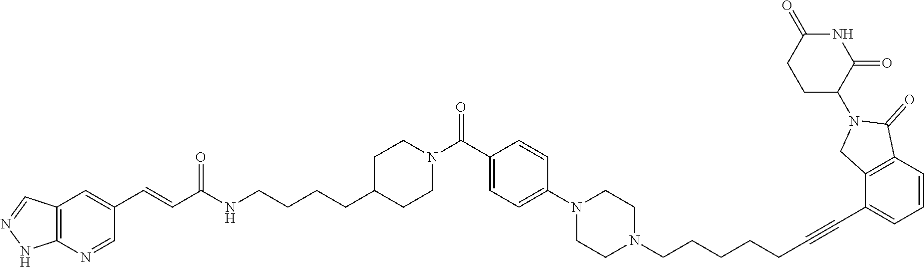 [SMILES: O=C(/C=C/c1cnc2[nH]ncc2c1)NCCCCC1CCN(C(=O)c2ccc(N3CCN(CCCCCC#Cc4cccc5c4CN(C4CCC(=O)NC4=O)C5=O)CC3)cc2)CC1] | (E)-N-(4-(1-(4-(4-(7-(2- (2,6-dioxopiperidin-3- yl)-1-oxoisoindolin-4- yl)hept-6-yn-1- yl)piperazin-1- yl)benzoyl)piperidin- 4-yl)butyl)-3-(1H- pyrazolo[3,4-b] pyridin- 5-yl)acrylamide | (E)-N-(4-(1-(4-(4-(7- (2-(2,6- dioxopiperidin- 3-yl)-1- oxoisoindolin- 4-yl)hept-6-yn-1- yl)piperazin-1- yl)benzoyl) piperidin-4- yl)butyl)-3-(1H- pyrazolo[3,4-b] pyridin- 5-yl)acrylamide |
| | 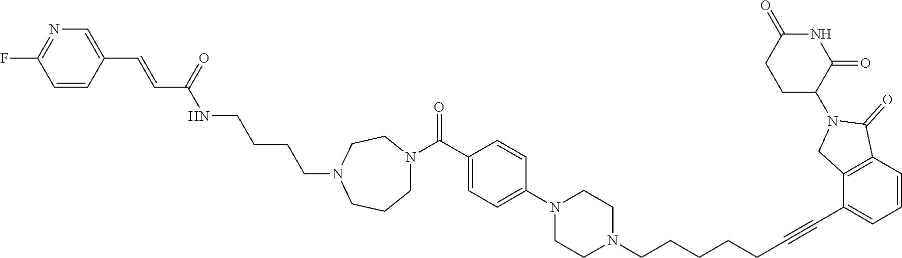 [SMILES: O=C(/C=C/c1ccc(F)nc1)NCCCCN1CCCN(C(=O)c2ccc(N3CCN(CCCCCC#Cc4cccc5c4CN(C4CCC(=O)NC4=O)C5=O)CC3)cc2)CC1] | (E)-N-(4-(4-(4-(4-(7-(2- (2,6-dioxopiperidin-3- yl)-1-oxoisoindolin-4- yl)hept-6-yn-1- yl)piperazin-1- yl)benzoyl)-1,4- diazepan-1-yl)butyl)- 3-(6-fluoropyridin-3- yl)acrylamide | (E)-N-(4-(4-(4-(4-(7- (2-(2,6- dioxopiperidin- 3-yl)-1- oxoisoindolin- 4-yl)hept-6-yn-1- yl)piperazin-1- yl)benzoyl)-1,4- diazepan-1-yl) butyl)-3- (6-fluoropyridin-3- yl)acrylamide |
| | 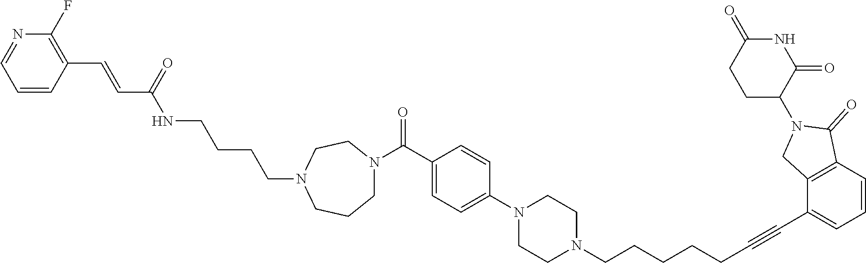 [SMILES: O=C(/C=C/c1cccnc1F)NCCCCN1CCCN(C(=O)c2ccc(N3CCN(CCCCCC#Cc4cccc5c4CN(C4CCC(=O)NC4=O)C5=O)CC3)cc2)CC1] | (E)-N-(4-(4-(4-(4-(7-(2- (2,6-dioxopiperidin-3- yl)-1-oxoisoindolin-4- yl)hept-6-yn-1- yl)piperazin-1 - yl)benzoyl)-1,4- diazepan-1-yl)butyl)- 3-(2-fluoropyridin-3- yl)acrylamide | (E)-N-(4-(4-(4-(4-(7- (2-(2,6- dioxopiperidin- 3-yl)-1- oxoisoindolin- 4-yl)hept-6-yn-1- yl)piperazin-1- yl)benzoyl)-1,4- diazepan-1-yl) butyl)-3- (2-fluoropyridin-3- yl)acrylamide |
| | 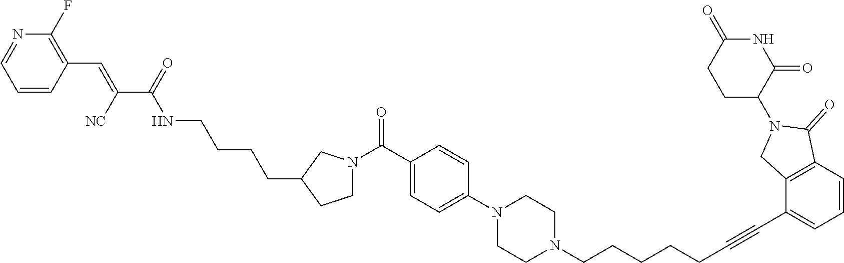 [SMILES: N#C/C(=C\c1cccnc1F)C(=O)NCCCCC1CCN(C(=O)c2ccc(N3CCN(CCCCCC#Cc4cccc5c4CN(C4CCC(=O)NC4=O)C5=O)CC3)cc2)C1] | (E)-2-cyano-N-(4-(1-(4- (4-(7-(2-(2,6- dioxopiperidin-3-yl)-1- oxoisoindolin-4-yl) hept-6-yn-1-yl) piperazin-1- yl)benzoyl)pyrrolidin- 3-yl)butyl)-3-(2- fluoropyridin-3- yl)acrylamide | (E)-2-cyano-N-(4-(1- (4-(4-(7-(2-(2,6- dioxopiperidin-3- yl)-1-oxoisoindolin- 4-yl)hept-6-yn-1- yl)piperazin-1- yl)benzoyl) pyrrolidin- 3-yl)butyl)-3-(2- fluoropyridin-3- yl)acrylamide |
| | 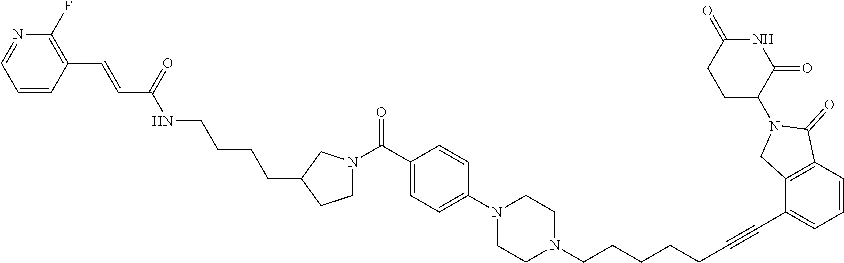 [SMILES: O=C(/C=C/c1cccnc1F)NCCCCC1CCN(C(=O)c2ccc(N3CCN(CCCCCC#Cc4cccc5c4CN(C4CCC(=O)NC4=O)C5=O)CC3)cc2)C1] | (E)-N-(4-(1-(4-(4-(7-(2- (2,6-dioxopiperidin-3- yl)-1-oxoisoindolin-4- yl)hept-6-yn-1- yl)piperazin-1- yl)benzoyl)pyrrolidin- 3-yl)butyl)-3-(2- fluoropyridin-3- yl)acrylamide | (E)-N-(4-(1-(4-(4-(7- (2-(2,6- dioxopiperidin- 3-yl)-1- oxoisoindolin- 4-yl)hept-6-yn-1- yl)piperazin-1- yl)benzoyl) pyrrolidin- 3-yl)butyl)-3-(2- fluoropyridin-3- yl)acrylamide |
| | 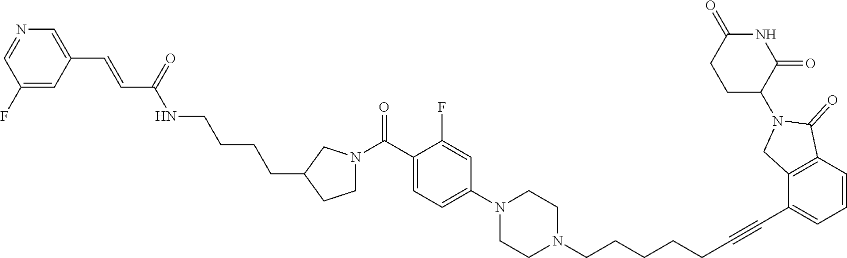 [SMILES: O=C(/C=C/c1cncc(F)c1)NCCCCC1CCN(C(=O)c2ccc(N3CCN(CCCCCC#Cc4cccc5c4CN(C4CCC(=O)NC4=O)C5=O)CC3)cc2F)C1] | (E)-N-(4-(1-(4-(4-(7-(2- (2,6-dioxopiperidin-3- yl)-1-oxoisoindolin-4- yl)hept-6-yn-1- yl)piperazin-1-yl)-2- fluorobenzoyl) pyrrolidin-3- yl)butyl)-3-(5- fluoropyridin-3- yl)acrylamide | (E)-N-(4-(1-(4-(4-(7- (2-(2,6- dioxopiperidin- 3-yl)-1- oxoisoindolin- 4-yl)hept-6-yn-1- yl)piperazin-1-yl)-2- fluorobenzoyl) pyrrolidin-3- yl)butyl)-3-(5- fluoropyridin-3- yl)acrylamide |
| | 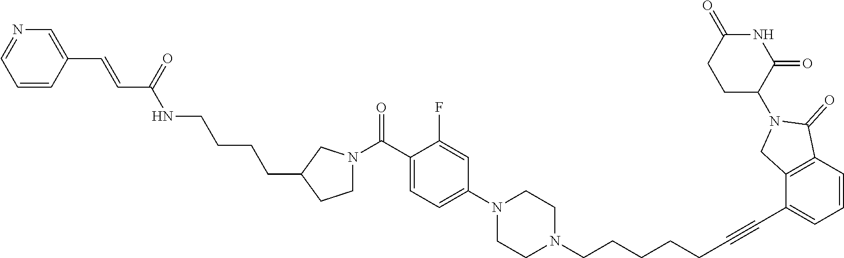 [SMILES: O=C(/C=C/c1cccnc1)NCCCCC1CCN(C(=O)c2ccc(N3CCN(CCCCCC#Cc4cccc5c4CN(C4CCC(=O)NC4=O)C5=O)CC3)cc2F)C1] | (E)-N-(4-(1-(4-(4-(7-(2- (2,6-dioxopiperidin-3- yl)-1-oxoisoindolin-4- yl)hept-6-yn-1- yl)piperazin-1-yl)-2- fluorobenzoyl) pyrrolidin-3-yl)butyl)- 3-(pyridin- 3-yl)acrylamide | (E)-N-(4-(1-(4-(4-(7- (2-(2,6- dioxopiperidin- 3-yl)-1- oxoisoindolin- 4-yl)hept-6-yn-1- yl)piperazin-1-yl)-2- fluorobenzoyl) pyrrolidin-3- yl)butyl)-3- (pyridin-3- yl)acrylamide |
| | 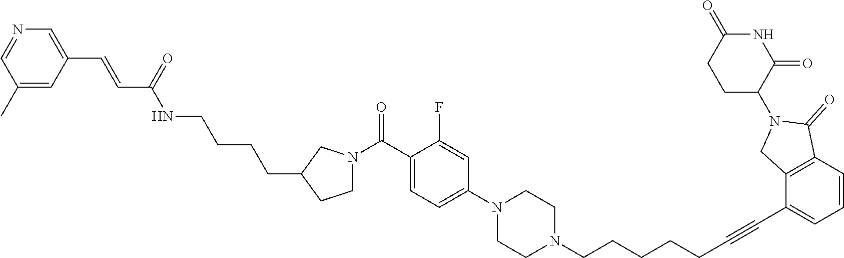 [SMILES: Cc1cncc(/C=C/C(=O)NCCCCC2CCN(C(=O)c3ccc(N4CCN(CCCCCC#Cc5cccc6c5CN(C5CCC(=O)NC5=O)C6=O)CC4)cc3F)C2)c1] | (E)-N-(4-(1-(4-(4-(7-(2- (2,6-dioxopiperidin-3- yl)-1-oxoisoindolin-4- yl)hept-6-yn-1- yl)piperazin-1-yl)-2- fluorobenzoyl) pyrrolidin-3- yl)butyl)-3-(5- methylpyridin-3- yl)acrylamide | (E)-N-(4-(1-(4-(4-(7- (2-(2,6- dioxopiperidin- 3-yl)-1- oxoisoindolin- 4-yl)hept-6-yn-1- yl)piperazin-1-yl)-2- fluorobenzoyl) pyrrolidin- 3-yl)butyl)-3-(5- methylpyridin-3- yl)acrylamide |
| | 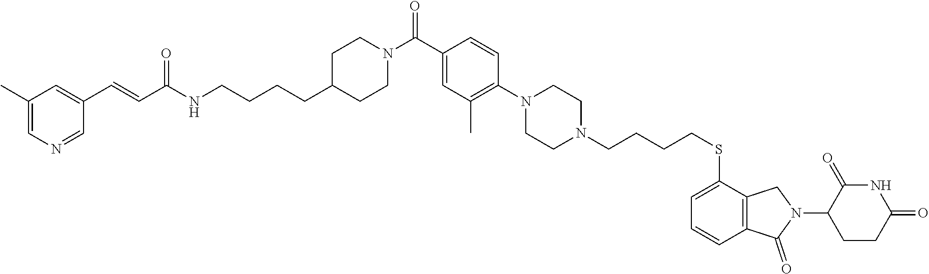 [SMILES: Cc1cncc(/C=C/C(=O)NCCCCC2CCN(C(=O)c3ccc(N4CCN(CCCCSc5cccc6c5CN(C5CCC(=O)NC5=O)C6=O)CC4)c(C)c3)CC2)c1] | (E)-N-(4-(1-(4-(4-(4- ((2-(2,6- dioxopiperidin- 3-yl)-1-oxoisoindolin- 4-yl)thio)butyl) piperazin-1-yl)-3- methylbenzoyl) piperidin-4-yl) butyl)-3-(5- methylpyridin-3- yl)acrylamide | (E)-N-(4-(1-(4-(4-(4- ((2-(2,6- dioxopiperidin- 3-yl)-1- oxoisoindolin-4- yl)thio)butyl) piperazin-1-yl)-3- methylbenzoyl) piperidin-4- yl)butyl)-3-(5- methylpyridin-3- yl)acrylamide |
| | 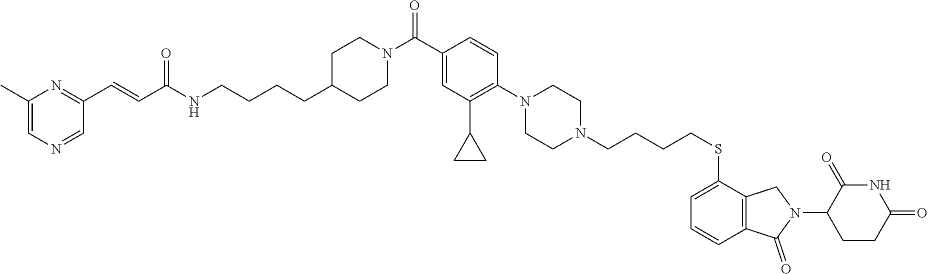 [SMILES: Cc1cncc(/C=C/C(=O)NCCCCC2CCN(C(=O)c3ccc(N4CCN(CCCCSc5cccc6c5CN(C5CCC(=O)NC5=O)C6=O)CC4)c(C4CC4)c3)CC2)n1] | (E)-N-(4-(1-(3- cyclopropyl- 4-(4-(4-((2- (2,6-dioxopiperidin-3- yl)-1-oxoisoindolin-4- yl)thio)butyl) piperazin-1-yl) benzoyl)piperidin- 4-yl)butyl)-3-(6- methylpyrazin-2- yl)acrylamide | (E)-N-(4-(1-(3- cyclopropyl- 4-(4-(4-((2-(2,6- dioxopiperidin-3-yl)- 1-oxoisoindolin-4- yl)thio)butyl) piperazin-1-yl) benzoyl)piperidin- 4-yl)butyl)-3-(6- methylpyrazin-2- yl)acrylamide |
| | 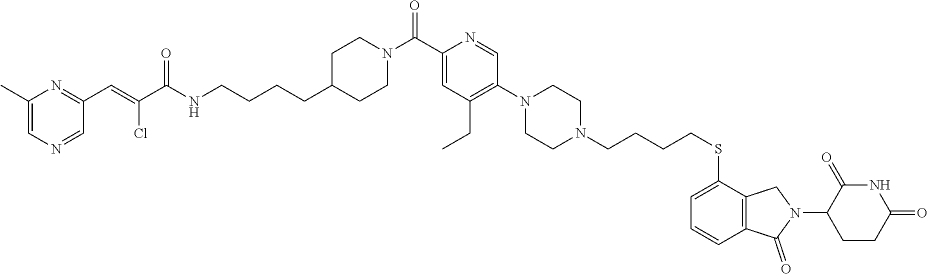 [SMILES: CCc1cc(C(=O)N2CCC(CCCCNC(=O)/C(Cl)=C/c3cncc(C)n3)CC2)ncc1N1CCN(CCCCSc2cccc3c2CN(C2CCC(=O)NC2=O)C3=O)CC1] | (Z)-2-chloro-N-(4-(1-(5- (4-(4-((2-(2,6- dioxopiperidin-3-yl)- 1-oxoisoindolin-4- yl)thio)butyl) piperazin-1-yl)-4- ethylpicolinoyl) piperidin-4-yl) butyl)-3-(6- methylpyrazin-2- yl)acrylamide | (Z)-2-chloro-N-(4-(1- (5-(4-(4-((2-(2,6- dioxopiperidin- 3-yl)-1- oxoisoindolin-4- yl)thio)butyl) piperazin-1-yl)-4- ethylpicolinoyl) piperidin-4-yl) butyl)-3-(6- methylpyrazin-2- yl)acrylamide |
| | 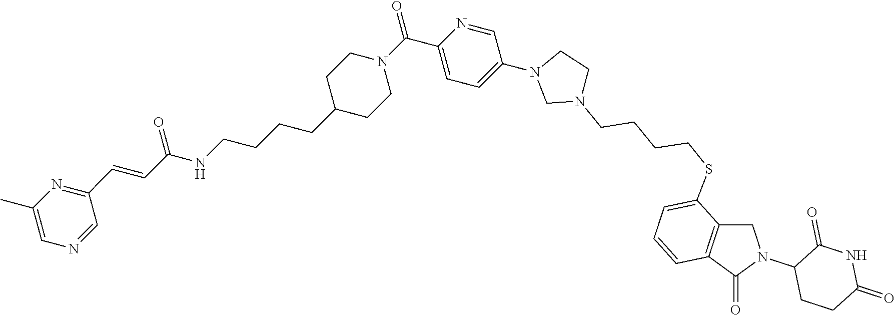 [SMILES: Cc1cncc(/C=C/C(=O)NCCCCC2CCN(C(=O)c3ccc(N4CCN(CCCCSc5cccc6c5CN(C5CCC(=O)NC5=O)C6=O)C4)cn3)CC2)n1] | (E)-N-(4-(1-(5-(3-(4- ((2-(2,6- dioxopiperidin- 3-yl)-1- oxoisoindolin-4- yl)thio)butyl) imidazolidin-1- yl)picolinoyl) piperidin- 4-yl)butyl)-3-(6- methylpyrazin-2- yl)acrylamide | (E)-N-(4-(1-(5-(3-(4- ((2-(2,6- dioxopiperidin- 3-yl)-1- oxoisoindolin-4- yl)thio)butyl) imidazolidin-1- yl)picolinoyl) piperidin- 4-yl)butyl)-3-(6- methylpyrazin-2- yl)acrylamide |
| | 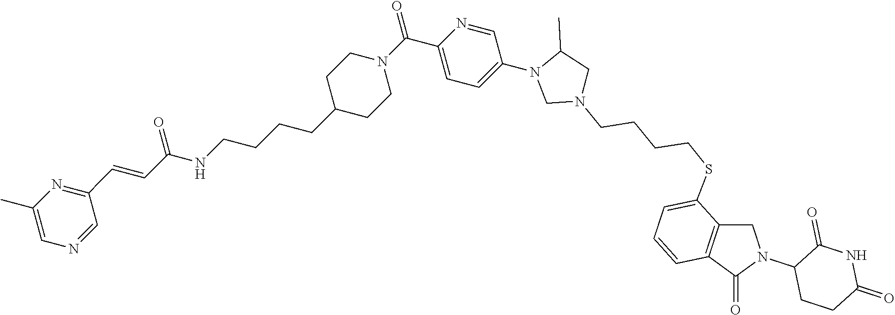 [SMILES: Cc1cncc(/C=C/C(=O)NCCCCC2CCN(C(=O)c3ccc(N4CN(CCCCSc5cccc6c5CN(C5CCC(=O)NC5=O)C6=O)CC4C)cn3)CC2)n1] | (E)-N-(4-(1-(5-(3-(4- ((2-(2,6- dioxopiperidin- 3-yl)-1- oxoisoindolin-4- yl)thio)butyl)-5- methylimidazolidin- 1-yl)picolinoyl) piperidin- 4-yl)butyl)-3-(6- methylpyrazin-2- yl)acrylamide | (E)-N-(4-(1-(5-(3-(4- ((2-(2,6- dioxopiperidin- 3-yl)-1- oxoisoindolin-4- yl)thio)butyl)-5- methylimidazolidin- 1-yl)picolinoyl) piperidin- 4-yl)butyl)-3-(6- methylpyrazin-2- yl)acrylamide |
| | 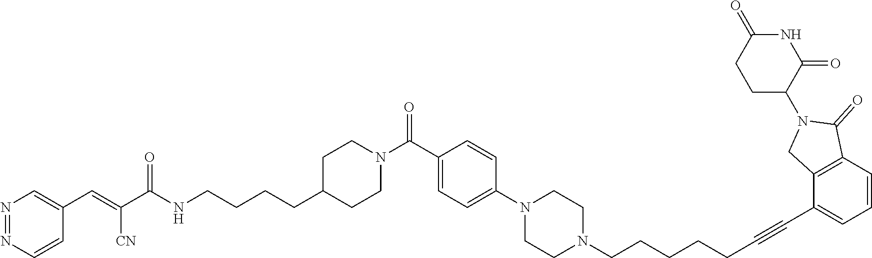 [SMILES: N#C/C(=C\c1ccnnc1)C(=O)NCCCCC1CCN(C(=O)c2ccc(N3CCN(CCCCCC#Cc4cccc5c4CN(C4CCC(=O)NC4=O)C5=O)CC3)cc2)CC1] | (E)-2-cyano-N-(4-(1-(4- (4-(7-(2-(2,6- dioxopiperidin-3-yl)- 1-oxoisoindolin-4- yl)hept- 6-yn-1-yl)piperazin-1- yl)benzoyl)piperidin-4- yl)butyl)-3-(pyridazin- 4-yl)acrylamide | (E)-2-cyano-N-(4-(1- (4-(4-(7-(2-(2,6- dioxopiperidin- 3-yl)-1- oxoisoindolin-4- yl)hept-6-yn-1- yl)piperazin-1- yl)benzoyl)piperidin- 4-yl)butyl)- 3-(pyridazin- 4-yl)acrylamide |
| | 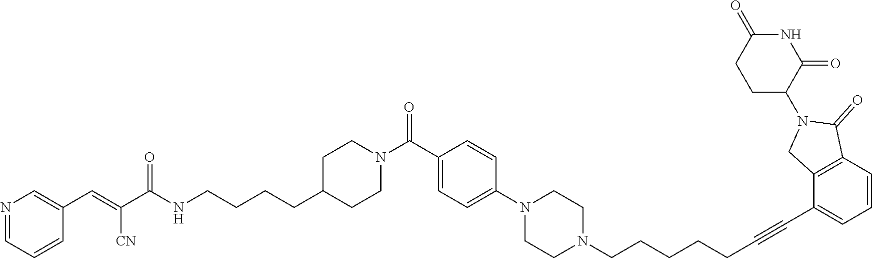 [SMILES: N#C/C(=C\c1cccnc1)C(=O)NCCCCC1CCN(C(=O)c2ccc(N3CCN(CCCCCC#Cc4cccc5c4CN(C4CCC(=O)NC4=O)C5=O)CC3)cc2)CC1] | (E)-2-cyano-N-(4-(1-(4- (4-(7-(2-(2,6- dioxopiperidin-3-yl)-1- oxoisoindolin-4-yl) hept-6-yn-1- yl)piperazin-1- yl)benzoyl)piperidin- 4-yl)butyl)-3-(pyridin- 3-yl)acrylamide | (E)-2-cyano-N-(4-(1- (4-(4-(7-(2-(2,6- dioxopiperidin- 3-yl)-1- oxoisoindolin-4- yl)hept-6-yn-1- yl)piperazin-1- yl)benzoyl) piperidin-4- yl)butyl)-3- (pyridin-3- yl)acrylamide |
| | 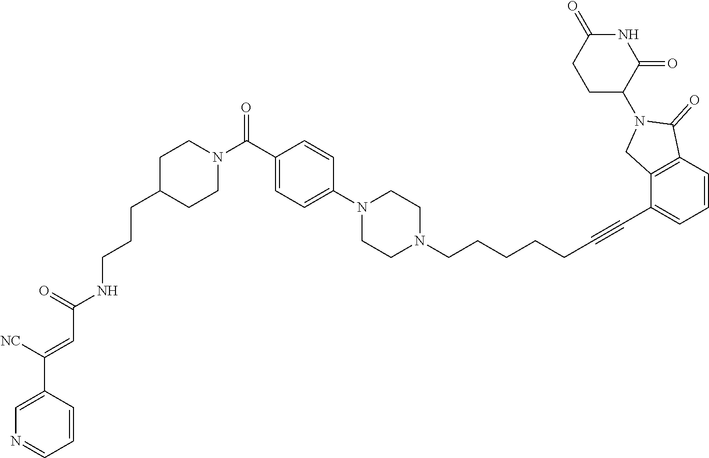 [SMILES: N#C/C(=C\C(=O)NCCCC1CCN(C(=O)c2ccc(N3CCN(CCCCCC#Cc4cccc5c4CN(C4CCC(=O)NC4=O)C5=O)CC3)cc2)CC1)c1cccnc1] | (Z)-3-cyano-N-(3-(1-(4- (4-(7-(2-(2,6- dioxopiperidin-3-yl)- 1-oxoisoindolin-4-yl) hept-6-yn-1-yl) piperazin-1- yl)benzoyl)piperidin- 4-yl)propyl)- 3-(pyridin-3- yl)acrylamide | (Z)-3-cyano-N-(3-(1- (4-(4-(7-(2-(2,6- dioxopiperidin- 3-yl)-1- oxoisoindolin-4- yl)hept-6-yn-1- yl)piperazin-1- yl)benzoyl)piperidin- 4-yl)propyl)- 3-(pyridin- 3-yl)acrylamide |
| | 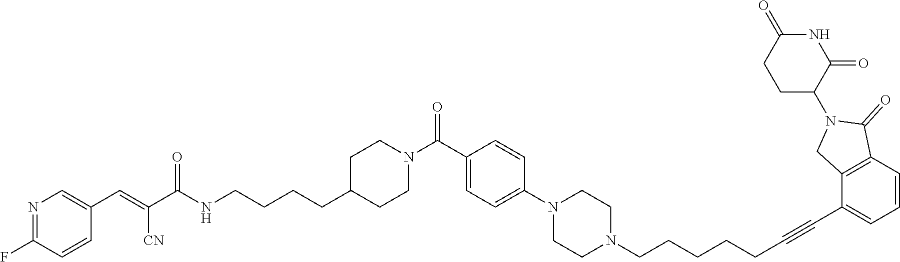 [SMILES: N#C/C(=C\c1ccc(F)nc1)C(=O)NCCCCC1CCN(C(=O)c2ccc(N3CCN(CCCCCC#Cc4cccc5c4CN(C4CCC(=O)NC4=O)C5=O)CC3)cc2)CC1] | (E)-2-cyano-N-(4-(1-(4- (4-(7-(2-(2,6- dioxopiperidin-3-yl)-1- oxoisoindolin-4-yl) hept-6-yn-1-yl) piperazin-1- yl)benzoyl)piperidin- 4-yl)butyl)-3-(6- fluoropyridin-3- yl)acrylamide | (E)-2-cyano-N-(4-(1- (4-(4-(7-(2-(2,6- dioxopiperidin-3-yl)- 1 -oxoisoindolin-4- yl)hept-6-yn-1- yl)piperazin-1- yl)benzoyl)piperidin- 4-yl)butyl)-3-(6- fluoropyridin-3- yl)acrylamide |
| | 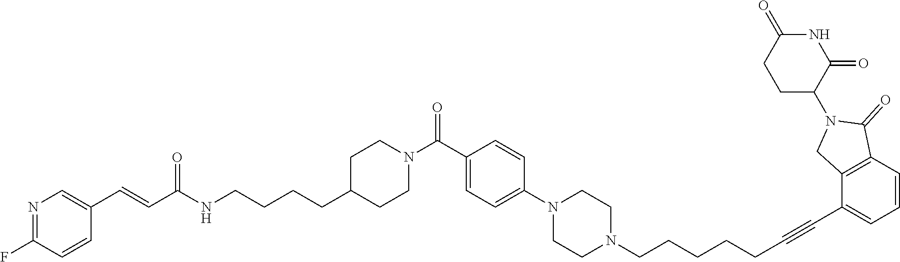 [SMILES: O=C(/C=C/c1ccc(F)nc1)NCCCCC1CCN(C(=O)c2ccc(N3CCN(CCCCCC#Cc4cccc5c4CN(C4CCC(=O)NC4=O)C5=O)CC3)cc2)CC1] | (E)-N-(4-(1-(4-(4-(7-(2- (2,6-dioxopiperidin-3- yl)-1-oxoisoindolin-4- yl)hept-6-yn-1- yl)piperazin-1- yl)benzoyl)piperidin- 4-yl)butyl)-3-(6- fluoropyridin-3- yl)acrylamide | (E)-N-(4-(1-(4-(4-(7- (2-(2,6- dioxopiperidin- 3-yl)-1- oxoisoindolin- 4-yl)hept-6-yn-1- yl)piperazin-1- yl)benzoyl) piperidin-4- yl)butyl)-3-(6- fluoropyridin-3- yl)acrylamide |
| | 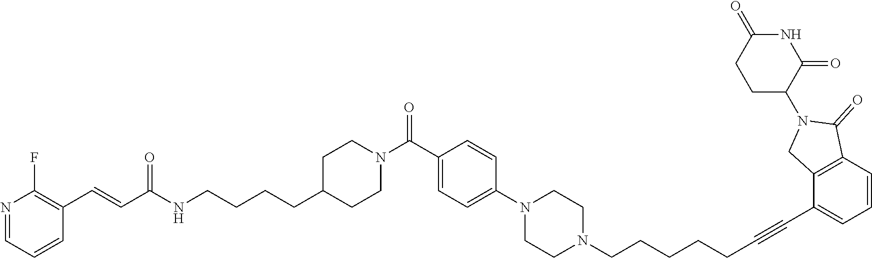 [SMILES: O=C(/C=C/c1cccnc1F)NCCCCC1CCN(C(=O)c2ccc(N3CCN(CCCCCC#Cc4cccc5c4CN(C4CCC(=O)NC4=O)C5=O)CC3)cc2)CC1] | (E)-N-(4-(1-(4-(4-(7-(2- (2,6-dioxopiperidin-3- yl)-1-oxoisoindolin-4- yl)hept-6-yn-1- yl)piperazin-1- yl)benzoyl)piperidin- 4-yl)butyl)-3-(2- fluoropyridin-3- yl)acrylamide | (E)-N-(4-(1-(4-(4-(7- (2-(2,6- dioxopiperidin- 3-yl)-1- oxoisoindolin- 4-yl)hept-6-yn-1- yl)piperazin-1- yl)benzoyl)piperidin- 4-yl)butyl)-3-(2- fluoropyridin-3- yl)acrylamide |
| | 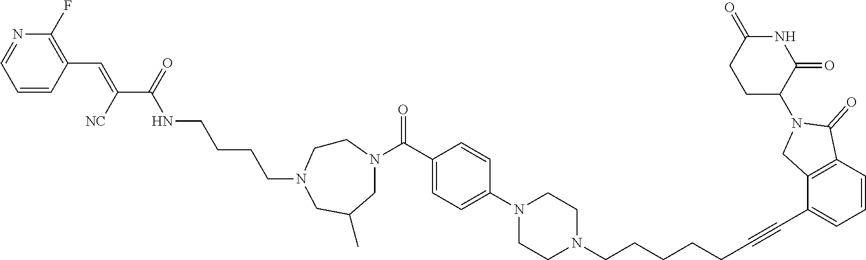 [SMILES: CC1CN(CCCCNC(=O)/C(C#N)=C/c2cccnc2F)CCN(C(=O)c2ccc(N3CCN(CCCCCC#Cc4cccc5c4CN(C4CCC(=O)NC4=O)C5=O)CC3)cc2)C1] | (E)-2-cyano-N-(4-(4-(4- (4-(7-(2-(2,6- dioxopiperidin- 3-yl)-1- oxoisoindolin-4- yl)hept-6-yn-1-yl) piperazin-1 - yl)benzoyl)- 6-methyl- 1,4-diazepan-1- yl)butyl)-3-(2- fluoropyridin-3- yl)acrylamide | (E)-2-cyano-N-(4-(4- (4-(4-(7-(2-(2,6- dioxopiperidin- 3-yl)-1- oxoisoindolin-4- yl)hept-6-yn-1- yl)piperazin-1- yl)benzoyl)- 6-methyl- 1,4-diazepan-1- yl)butyl)-3-(2- fluoropyridin-3- yl)acrylamide |
| | 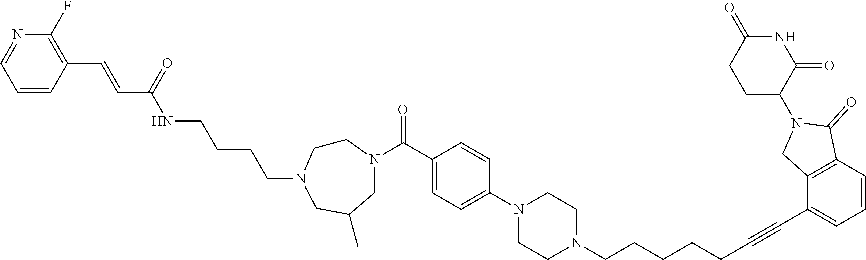 [SMILES: CC1CN(CCCCNC(=O)/C=C/c2cccnc2F)CCN(C(=O)c2ccc(N3CCN(CCCCCC#Cc4cccc5c4CN(C4CCC(=O)NC4=O)C5=O)CC3)cc2)C1] | (E)-N-(4-(4-(4-(4-(7-(2- (2,6-dioxopiperidin-3- yl)-1-oxoisoindolin-4- yl)hept-6-yn-1- yl)piperazin-1- yl)benzoyl)-6-methyl- 1,4-diazepan-1- yl)butyl)-3-(2- fluoropyridin-3- yl)acrylamide | (E)-N-(4-(4-(4-(4-(7- (2-(2,6- dioxopiperidin- 3-yl)-1- oxoisoindolin- 4-yl)hept-6-yn-1- yl)piperazin-1- yl)benzoyl)- 6-methyl- 1,4-diazepan-1- yl)butyl)-3-(2- fluoropyridin-3- yl)acrylamide |
| | 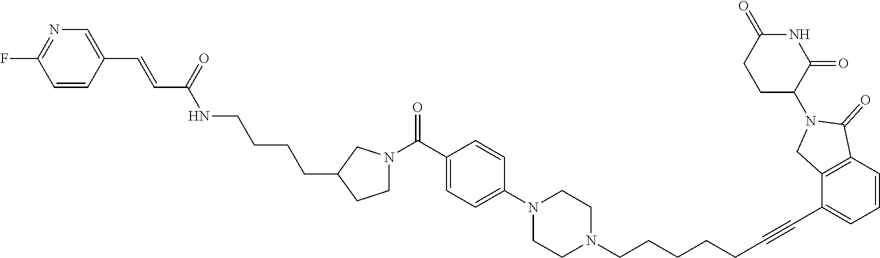 [SMILES: O=C(/C=C/c1ccc(F)nc1)NCCCCC1CCN(C(=O)c2ccc(N3CCN(CCCCCC#Cc4cccc5c4CN(C4CCC(=O)NC4=O)C5=O)CC3)cc2)C1] | (E)-N-(4-(1-(4-(4-(7-(2- (2,6-dioxopiperidin-3- yl)-1-oxoisoindolin-4- yl)hept-6-yn-1- yl)piperazin-1- yl)benzoyl)pyrrolidin- 3-yl)butyl)-3-(6- fluoropyridin-3- yl)acrylamide | (E)-N-(4-(1-(4-(4-(7- (2-(2,6- dioxopiperidin- 3-yl)-1- oxoisoindolin- 4-yl)hept-6-yn-1- yl)piperazin-1- yl)benzoyl) pyrrolidin- 3-yl)butyl)-3-(6- fluoropyridin-3- yl)acrylamide |
| | 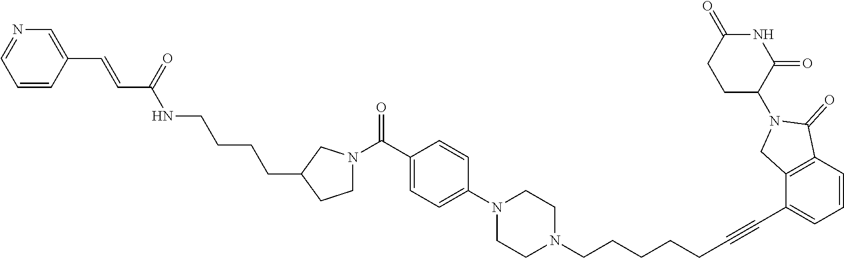 [SMILES: O=C(/C=C/c1cccnc1)NCCCCC1CCN(C(=O)c2ccc(N3CCN(CCCCCC#Cc4cccc5c4CN(C4CCC(=O)NC4=O)C5=O)CC3)cc2)C1] | (E)-N-(4-(1-(4-(4-(7-(2- (2,6-dioxopiperidin-3- yl)-1-oxoisoindolin-4- yl)hept-6-yn-1- yl)piperazin-1- yl)benzoyl)pyrrolidin- 3-yl)butyl)- 3-(pyridin-3- yl)acrylamide | (2-(2,6-dioxopiperidin- 3-yl)-1-oxoisoindolin- 4-yl)hept-6-yn-1- yl)piperazin-1- yl)benzoyl)pyrrolidin- 3-yl)butyl)- 3-(pyridin- 3-yl)acrylamide |
| | 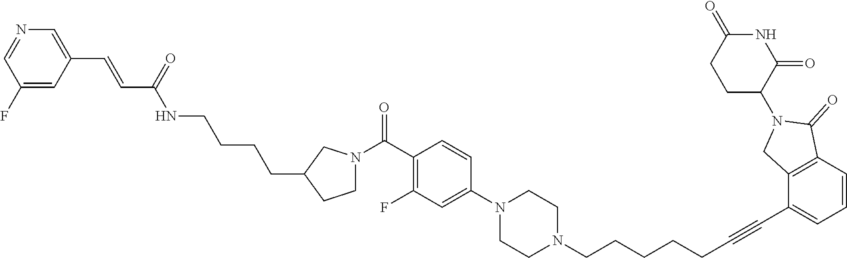 [SMILES: O=C(/C=C/c1cncc(F)c1)NCCCCC1CCN(C(=O)c2ccc(N3CCN(CCCCCC#Cc4cccc5c4CN(C4CCC(=O)NC4=O)C5=O)CC3)cc2F)C1] | (E)-N-(4-(1-(4-(4-(7-(2- (2,6-dioxopiperidin-3- yl)-1-oxoisoindolin-4- yl)hept-6-yn-1- yl)piperazin-1-yl)-2- fluorobenzoyl) pyrrolidin-3-yl) butyl)-3-(5- fluoropyridin-3- yl)acrylamide | (E)-N-(4-(1-(4-(4-(7- (2-(2,6- dioxopiperidin- 3-yl)-1- oxoisoindolin- 4-yl)hept-6-yn-1- yl)piperazin-1-yl)- 2-fluorobenzoyl) pyrrolidin-3- yl)butyl)-3-(5- fluoropyridin-3- yl)acrylamide |
| | 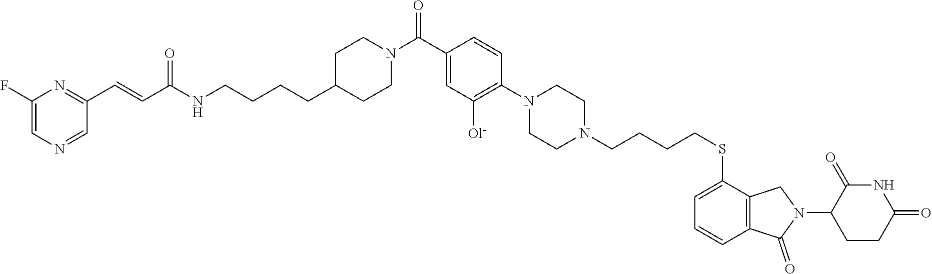 | (E)-N-(4-(1-(4-(4-(4- ((2-(2,6- dioxopiperidin- 3-yl)-1- oxoisoindolin-4- yl)thio)butyl) piperazin-1-yl)-3- hydroxybenzoyl) piperidin-4- yl)butyl)-3-(5- fluoropyridin-3- yl)acrylamide | (E)-N-(4-(1-(4-(4-(4- ((2-(2,6- dioxopiperidin- 3-yl)-1- oxoisoindolin-4- yl)thio)butyl) piperazin-1-yl)-3- hydroxybenzoyl) piperidin-4- yl)butyl)-3-(5- fluoropyridin-3- yl)acrylamide |
| | 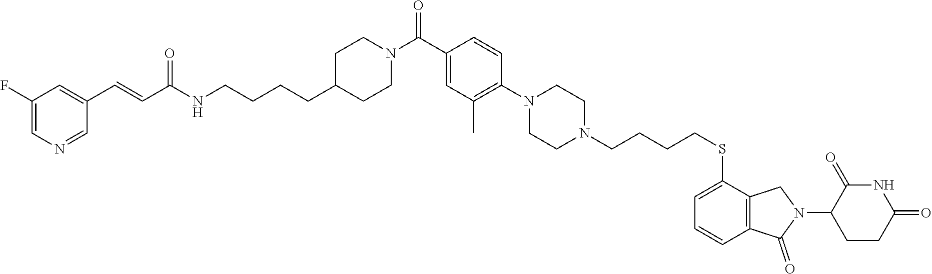 [SMILES: Cc1cc(C(=O)N2CCC(CCCCNC(=O)/C=C/c3cncc(F)c3)CC2)ccc1N1CCN(CCCCSc2cccc3c2CN(C2CCC(=O)NC2=O)C3=O)CC1] | (E)-N-(4-(1-(4-(4-(4- ((2-(2,6- dioxopiperidin- 3-yl)-1- oxoisoindolin-4- yl)thio)butyl) piperazin-1-yl)-3- methylbenzoyl) piperidin-4- yl)butyl)-3-(5- fluoropyridin-3- yl)acrylamide | (E)-N-(4-(1-(4-(4-(4- ((2-(2,6- dioxopiperidin- 3-yl)-1- oxoisoindolin-4- yl)thio)butyl) piperazin-1-yl)-3- methylbenzoyl) piperidin-4- yl)butyl)-3-(5- fluoropyridin-3- yl)acrylamide |
| | 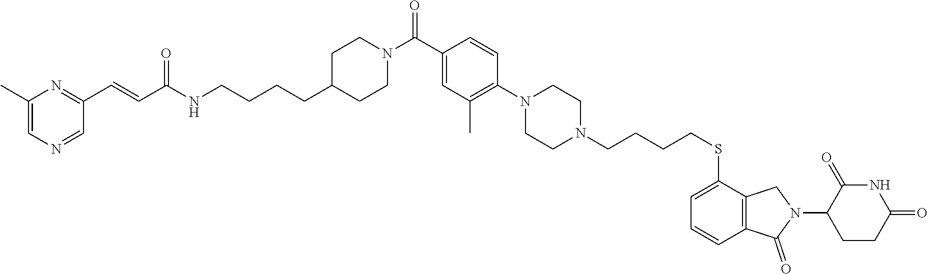 [SMILES: Cc1cncc(/C=C/C(=O)NCCCCC2CCN(C(=O)c3ccc(N4CCN(CCCCSc5cccc6c5CN(C5CCC(=O)NC5=O)C6=O)CC4)c(C)c3)CC2)n1] | (E)-N-(4-(1-(4-(4-(4- ((2-(2,6- dioxopiperidin- 3-yl)-1- oxoisoindolin-4- yl)thio)butyl) piperazin-1-yl)-3- methylbenzoyl) piperidin-4- yl)butyl)-3-(6- methylpyrazin-2- yl)acrylamide | (E)-N-(4-(1-(4-(4-(4- ((2-(2,6- dioxopiperidin- 3-yl)-1- oxoisoindolin-4- yl)thio)butyl) piperazin-1-yl)-3- methylbenzoyl) piperidin-4- yl)butyl)-3-(6- methylpyrazin-2- yl)acrylamide |
| | 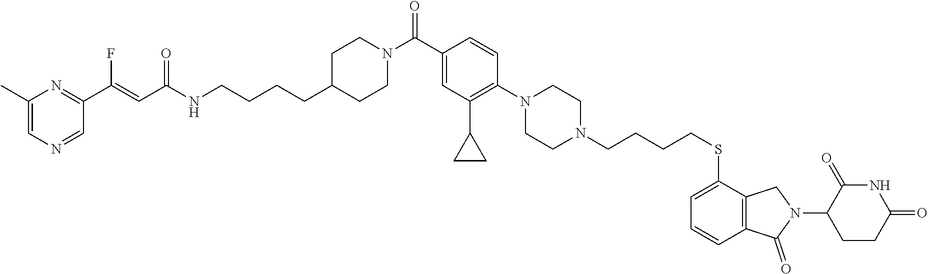 [SMILES: Cc1cncc(/C(F)=C/C(=O)NCCCCC2CCN(C(=O)c3ccc(N4CCN(CCCCSc5cccc6c5CN(C5CCC(=O)NC5=O)C6=O)CC4)c(C4CC4)c3)CC2)n1] | (Z)-N-(4-(1-(3- cyclopropyl- 4-(4-(4-((2- (2,6-dioxopiperidin- 3-yl)-1- oxoisoindolin-4- yl)thio)butyl) piperazin-1- yl)benzoyl) piperidin- 4-yl)butyl)- 3-fluoro-3- (6-methylpyrazin- 2-yl)acrylamide | (Z)-N-(4-(1-(3- cyclopropyl- 4-(4-(4-((2-(2,6- dioxopiperidin- 3-yl)-1- oxoisoindolin-4- yl)thio)butyl) piperazin-1-yl) benzoyl)piperidin- 4-yl)butyl)- 3-fluoro-3- (6-methylpyrazin- 2-yl)acrylamide |
| | 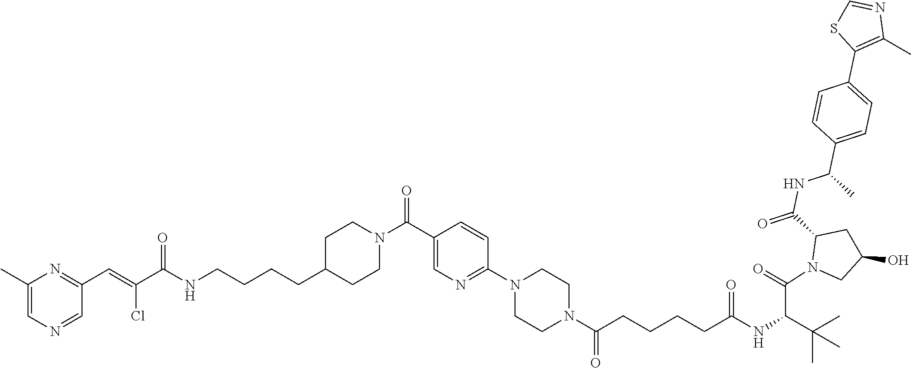 | (2S,4R)-1-((S)-2-(6-(4- (5-(4-(4-((Z)-2-chloro- 3-(6-methylpyrazin-2- yl)acrylamido)butyl) piperidine-1- carbonyl)pyridin-2- yl)piperazin-1-yl)-6- oxohexanamido)-3,3- dimethylbutanoyl)-4- hydroxy-N-((S)-1-(4- (4-methylthiazol-5- yl)phenyl)ethyl) pyrrolidine-2- carboxamide | (2S,4R)-1-((S)-2-(6-(4- (5-(4-(4-((Z)-2-chloro- 3-(6-methylpyrazin-2- yl)acrylamido)butyl) piperidine-1- carbonyl)pyridin-2- yl)piperazin-1-yl)-6- oxohexanamido)-3,3- dimethylbutanoyl)-4- hydroxy-N-((S)-1-(4- (4-methylthiazol-5- yl)phenyl)ethyl) pyrrolidine-2- carboxamide |
| | 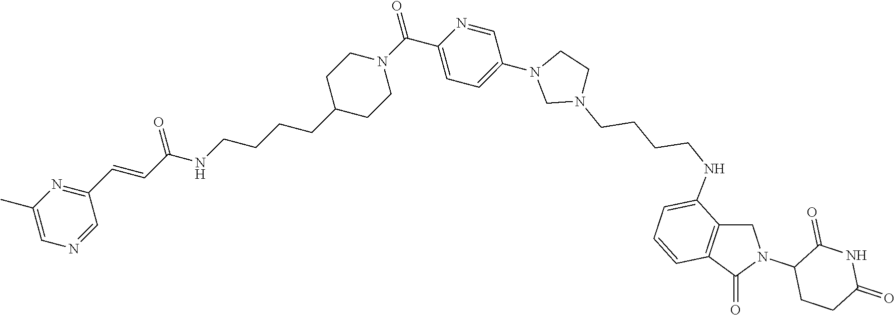 [SMILES: Cc1cncc(/C=C/C(=O)NCCCCC2CCN(C(=O)c3ccc(N4CCN(CCCCNc5cccc6c5CN(C5CCC(=O)NC5=O)C6=O)C4)cn3)CC2)n1] | (E)-N-(4-(1-(5-(3-(4- ((2-(2,6- dioxopiperidin- 3-yl)-1- oxoisoindolin-4- yl)amino)butyl) imidazolidin-1- yl)picolinoyl) piperidin- 4-yl)butyl)-3-(6- methylpyrazin-2- yl)acrylamide | (E)-N-(4-(1-(5-(3-(4- ((2-(2,6- dioxopiperidin- 3-yl)-1- oxoisoindolin-4- yl)amino)butyl) imidazolidin-1- yl)picolinoyl) piperidin- 4-yl)butyl)-3-(6- methylpyrazin-2- yl)acrylamide |
| | 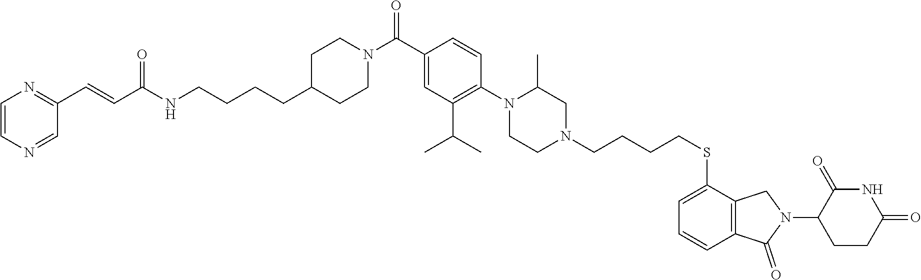 [SMILES: CC(C)c1cc(C(=O)N2CCC(CCCCNC(=O)/C=C/c3cnccn3)CC2)ccc1N1CCN(CCCCSc2cccc3c2CN(C2CCC(=O)NC2=O)C3=O)CC1C] | (E)-N-(4-(1-(4-(4-(4- ((2-(2,6- dioxopiperidin- 3-yl)-1- oxoisoindolin-4- yl)thio)butyl)-2- methylpiperazin- 1-yl)-3-isopropyl benzoyl)piperidin- 4-yl)butyl)-3- (pyrazin-2- yl)acrylamide | (E)-N-(4-(1-(4-(4-(4- ((2-(2,6- dioxopiperidin- 3-yl)-1- oxoisoindolin-4- yl)thio)butyl)-2- methylpiperazin- 1-yl)-3- isopropylbenzoyl) piperidin-4- yl)butyl)-3- (pyrazin-2- yl)acrylamide |
| | 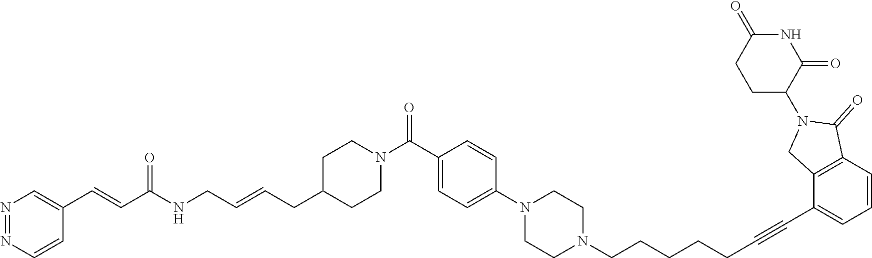 [SMILES: O=C(/C=C/c1ccnnc1)NC/C=C/CC1CCN(C(=O)c2ccc(N3CCN(CCCCCC#Cc4cccc5c4CN(C4CCC(=O)NC4=O)C5=O)CC3)cc2)CC1] | (E)-N-((E)-4-(1-(4-(4- (7-(2-(2,6- dioxopiperidin- 3-yl)-1- oxoisoindolin-4-yl) hept-6-yn-1- yl)piperazin-1- yl)benzoyl)piperidin- 4-yl)but-2-en-1-yl)-3- (pyridazin-4- yl)acrylamide | (E)-N-((E)-4-(1-(4-(4- (7-(2-(2,6- dioxopiperidin- 3-yl)-1- oxoisoindolin-4- yl)hept-6-yn-1- yl)piperazin-1- yl)benzoyl)piperidin- 4-yl)but-2-en-1-yl)-3- (pyridazin-4- yl)acrylamide |
| | 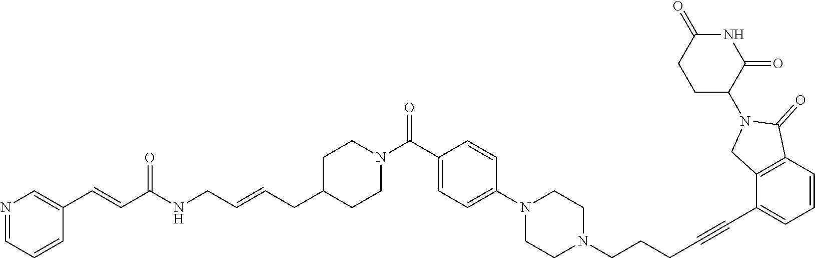 [SMILES: O=C(/C=C/c1cccnc1)NC/C=C/CC1CCN(C(=O)c2ccc(N3CCN(CCCC#Cc4cccc5c4CN(C4CCC(=O)NC4=O)C5=O)CC3)cc2)CC1] | (E)-N-((E)-4-(1-(4-(4- (7-(2-(2,6- dioxopiperidin- 3-yl)-1- oxoisoindolin-4-yl) hept-6-yn-1- yl)piperazin-1- yl)benzoyl)piperidin- 4-yl)but-2-en-1-yl)-3- (pyridin-3- yl)acrylamide | (E)-N-((E)-4-(1-(4-(4- (7-(2-(2,6- dioxopiperidin-3-yl)- 1-oxoisoindolin-4- yl)hept-6-yn-1- yl)piperazin-1- yl)benzoyl)piperidin- 4-yl)but-2-en-1-yl)-3- (pyridin-3- yl)acrylamide |
| | 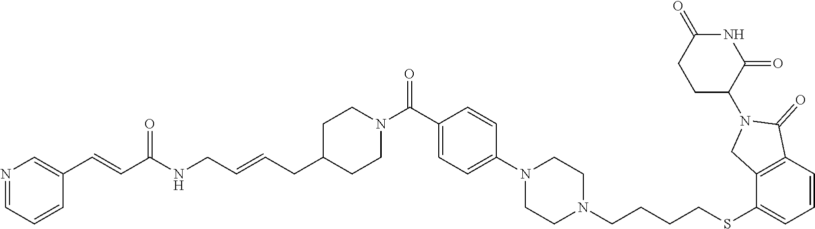 [SMILES: O=C(/C=C/c1cccnc1)NC/C=C/CC1CCN(C(=O)c2ccc(N3CCN(CCCCSc4cccc5c4CN(C4CCC(=O)NC4=O)C5=O)CC3)cc2)CC1] | (E)-N-((E)-4-(1-(4-(4- (4-((2-(2,6- dioxopiperidin- 3-yl)-1- oxoisoindolin-4- yl)thio)butyl) piperazin- 1-yl)benzoyl) piperidin- 4-yl)but-2-en-1- yl)-3-(pyridin-3- yl)acrylamide | (E)-N-((E)-4-(1-(4-(4- (4-((2-(2,6- dioxopiperidin- 3-yl)-1- oxoisoindolin-4- yl)thio)butyl) piperazin- 1-yl)benzoyl) piperidin- 4-yl)but-2-en-1- yl)-3-(pyridin-3- yl)acrylamide |
| | 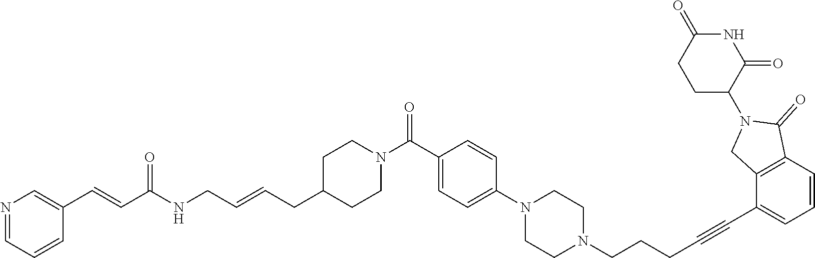 [SMILES: O=C(/C=C/c1cccnc1)NC/C=C/CC1CCN(C(=O)c2ccc(N3CCN(CCCC#Cc4cccc5c4CN(C4CCC(=O)NC4=O)C5=O)CC3)cc2)CC1] | (E)-N-((E)-4-(1-(4-(4- (5-(2-(2,6- dioxopiperidin- 3-yl)-1- oxoisoindolin-4- yl)pent-4-yn-1- yl)piperazin-1 - yl)benzoyl)piperidin- 4-yl)but-2-en-1- yl)-3-(pyridin-3- yl)acrylamide | (E)-N-((E)-4-(1-(4-(4- (5-(2-(2,6- dioxopiperidin -3-yl)-1- oxoisoindolin-4- yl)pent-4-yn-1- yl)piperazin-1- yl)benzoyl) piperidin-4- yl)but-2-en-1-yl)-3- (pyridin-3- yl)acrylamide |
| | 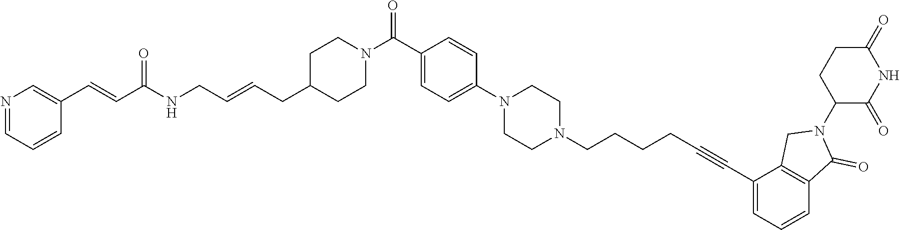 [SMILES: O=C(/C=C/c1cccnc1)NC/C=C/CC1CCN(C(=O)c2ccc(N3CCN(CCCCC#Cc4cccc5c4CN(C4CCC(=O)NC4=O)C5=O)CC3)cc2)CC1] | (E)-N-((E)-4-(1-(4-(4- (6-(2-(2,6- dioxopiperidin- 3-yl)-1- oxoisoindolin-4-yl) hex-5-yn-1- yl)piperazin-1- yl)benzoyl)piperidin- 4-yl)but-2-en-1-yl)-3- (pyridin-3- yl)acrylamide | (E)-N-((E)-4-(1-(4-(4- (6-(2-(2,6- dioxopiperidin- 3-yl)-1- oxoisoindolin-4-yl) hex-5-yn-1-yl) piperazin-1- yl)benzoyl) piperidin-4- yl)but-2-en-1-yl)-3- (pyridin-3- yl)acrylamide |
| | 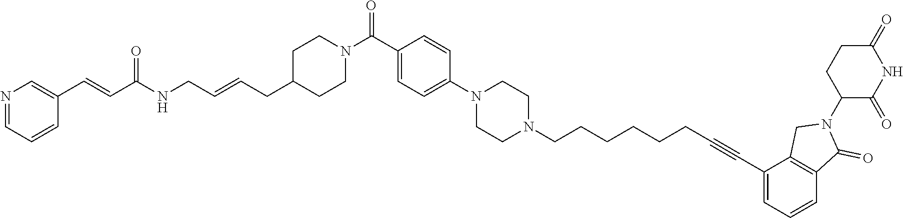 [SMILES: O=C(/C=C/c1cccnc1)NC/C=C/CC1CCN(C(=O)c2ccc(N3CCN(CCCCCCC#Cc4cccc5c4CN(C4CCC(=O)NC4=O)C5=O)CC3)cc2)CC1] | (E)-N-((E)-4-(1-(4-(4- (8-(2-(2,6- dioxopiperidin- 3-yl)-1- oxoisoindolin-4- yl)oct-7-yn-1- yl)piperazin-1- yl)benzoyl) piperidin-4- yl)but-2-en-1-yl)-3- (pyridin-3- yl)acrylamide | (E)-N-((E)-4-(1-(4-(4- (8-(2-(2,6- dioxopiperidin- 3-yl)-1- oxoisoindolin-4- yl)oct-7-yn-1- yl)piperazin-1- yl)benzoyl) piperidin-4- yl)but-2-en-1-yl)-3- (pyridin-3- yl)acrylamide |
| | 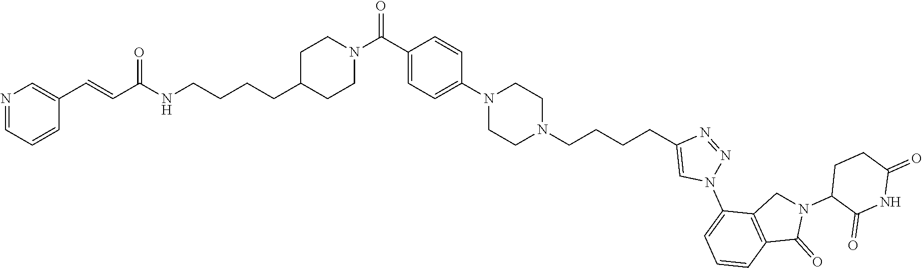 [SMILES: O=C(/C=C/c1cccnc1)NCCCCC1CCN(C(=O)c2ccc(N3CCN(CCCCc4cn(-c5cccc6c5CN(C5CCC(=O)NC5=O)C6=O)nn4)CC3)cc2)CC1] | (E)-N-(4-(1-(4-(4-(4-(1- (2-(2,6-dioxopiperidin- 3-yl)-1-oxoisoindolin- 4-yl)-1H-1,2,3- triazol-4- yl)butyl)piperazin-1- yl)benzoyl)piperidin- 4-yl)butyl)- 3-(pyridin-3- yl)acrylamide | (E)-N-(4-(1-(4-(4-(4- (1-(2-(2,6- dioxopiperidin- 3-yl)-1- oxoisoindolin-4-yl)- 1H-1,2,3-triazol-4- yl)butyl)piperazin-1- yl)benzoyl)piperidin- 4-yl)butyl)- 3-(pyridin-3- yl)acrylamide |
| | 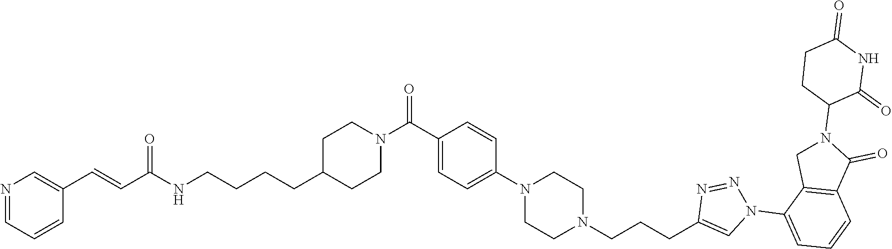 [SMILES: O=C(/C=C/c1cccnc1)NCCCCC1CCN(C(=O)c2ccc(N3CCN(CCCc4cn(-c5cccc6c5CN(C5CCC(=O)NC5=O)C6=O)nn4)CC3)cc2)CC1] | (E)-N-(4-(1-(4-(4-(3-(1- (2-(2,6-dioxopiperidin- 3-yl)-1-oxoisoindolin- 4-yl)-1H-1,2,3- triazol-4-yl)propyl) piperazin-1- yl)benzoyl) piperidin-4- yl)butyl)-3-(pyridin- 3-yl)acrylamide | (E)-N-(4-(1-(4-(4-(3- (1-(2-(2,6- dioxopiperidin- 3-yl)-1- oxoisoindolin-4-yl)- 1H-1,2,3-triazol-4- yl)propyl)piperazin- 1-yl)benzoyl) piperidin-4- yl)butyl)-3-(pyridin- 3-yl)acrylamide |
| | 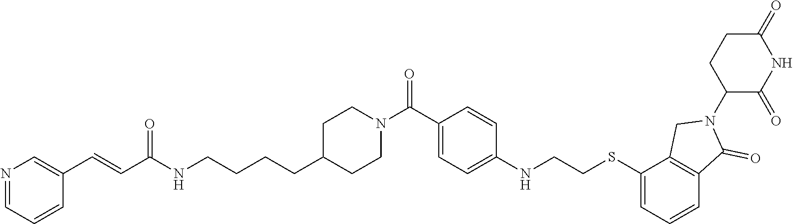 [SMILES: O=C(/C=C/c1cccnc1)NCCCCC1CCN(C(=O)c2ccc(NCCSc3cccc4c3CN(C3CCC(=O)NC3=O)C4=O)cc2)CC1] | (E)-N-(4-(1-(4-((2-((2- (2,6-dioxopiperidin-3- yl)-1-oxoisoindolin-4- yl)thio)ethyl)amino) benzoyl)piperidin-4- yl)butyl)-3-(pyridin- 3-yl)acrylamide | (E)-N-(4-(1-(4-((2-((2- (2,6-dioxopiperidin-3- yl)-1-oxoisoindolin-4- yl)thio)ethyl)amino) benzoyl)piperidin-4- yl)butyl)-3-(pyridin- 3-yl)acrylamide |
| | 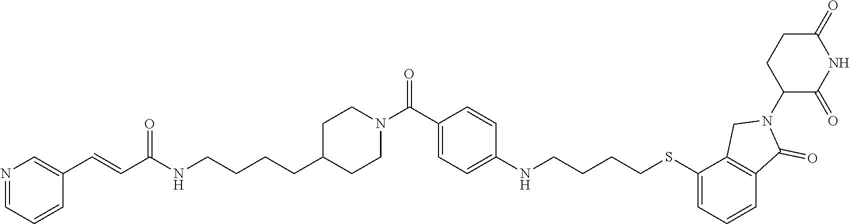 [SMILES: O=C(/C=C/c1cccnc1)NCCCCC1CCN(C(=O)c2ccc(NCCCCSc3cccc4c3CN(C3CCC(=O)NC3=O)C4=O)cc2)CC1] | (E)-N-(4-(1-(4-((4-((2- (2,6-dioxopiperidin-3- yl)-1-oxoisoindolin-4- yl)thio)butyl)amino) benzoyl)piperidin-4- yl)butyl)-3-(pyridin- 3-yl)acrylamide | (E)-N-(4-(1-(4-((4-((2- (2,6-dioxopiperidin-3- yl)-1-oxoisoindolin-4- yl)thio)butyl)amino) benzoyl)piperidin-4- yl)butyl)-3-(pyridin- 3-yl)acrylamide |
| | 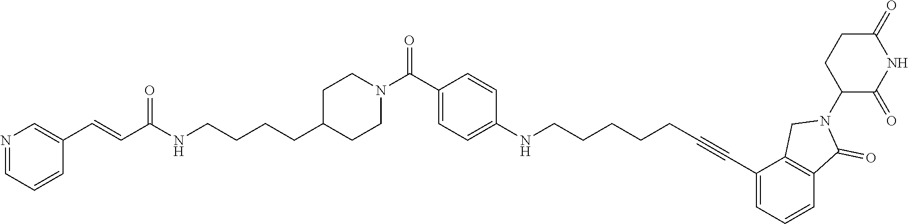 [SMILES: O=C(/C=C/c1cccnc1)NCCCCC1CCN(C(=O)c2ccc(NCCCCCC#Cc3cccc4c3CN(C3CCC(=O)NC3=O)C4=O)cc2)CC1] | (E)-N-(4-(1-(4-((7-(2- (2,6-dioxopiperidin-3- yl)-1-oxoisoindolin-4- yl)hept-6-yn-1- yl)amino)benzoyl) piperidin-4-yl) butyl)-3- (pyridin-3- yl)acrylamide | (E)-N-(4-(1-(4-((7-(2- (2,6-dioxopiperidin-3- yl)-1-oxoisoindolin-4- yl)hept-6-yn-1- yl)amino)benzoyl) piperidin-4-yl) butyl)-3-(pyridin-3- yl)acrylamide |
| | 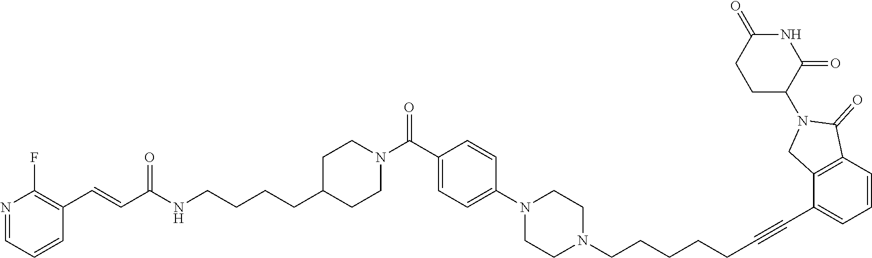 [SMILES: O=C(/C=C/c1cccnc1F)NCCCCC1CCN(C(=O)c2ccc(N3CCN(CCCCCC#Cc4cccc5c4CN(C4CCC(=O)NC4=O)C5=O)CC3)cc2)CC1] | (E)-N-(4-(1-(4-(4-(7-(2- (2,6-dioxopiperidin-3- yl)-1-oxoisoindolin-4- yl)hept-6-yn-1- yl)piperazin-1- yl)benzoyl)piperidin- 4-yl)butyl)-3-(2- fluoropyridin-3- yl)acrylamide | (E)-N-(4-(1-(4-(4-(7- (2-(2,6- dioxopiperidin- 3-yl)-1- oxoisoindolin- 4-yl)hept-6-yn-1- yl)piperazin-1- yl)benzoyl) piperidin-4- yl)butyl)-3-(2- fluoropyridin-3- yl)acrylamide |
| SIAI S633 097 | 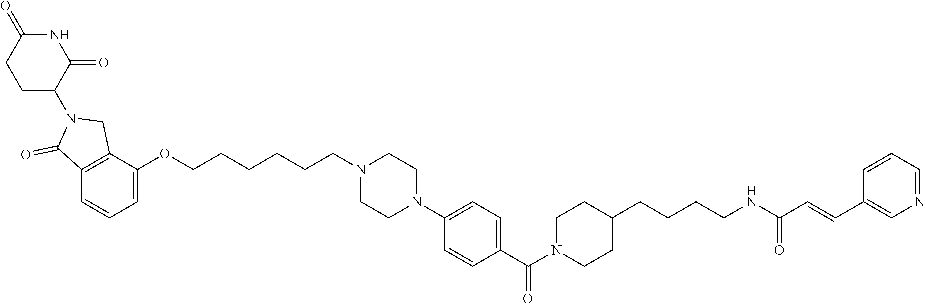 [SMILES: O=C(/C=C/c1cccnc1)NCCCCC1CCN(C(=O)c2ccc(N3CCN(CCCCCCOc4cccc5c4CN(C4CCC(=O)NC4=O)C5=O)CC3)cc2)CC1] | (E)-N-(4-(1-(4-(4-(6- ((2-(2,6- dioxopiperidin- 3-yl)-1- oxoisoindolin-4- yl)oxy)hexyl) piperazin- 1-yl)benzoyl) piperidin- 4-yl)butyl)- 3-(pyridin-3- yl)acrylamide | (E)-N-(4-(1-(4-(4-(6- ((2-(2,6- dioxopiperidin- 3-yl)-1- oxoisoindolin-4- yl)oxy)hexyl) piperazin- 1-yl)benzoyl) piperidin- 4-yl)butyl)- 3-(pyridin- 3-yl)acrylamide |
| SIAI S633 098 | 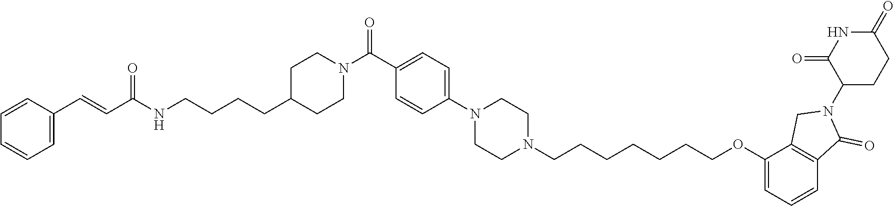 [SMILES: O=C(/C=C/c1ccccc1)NCCCCC1CCN(C(=O)c2ccc(N3CCN(CCCCCCCOc4cccc5c4CN(C4CCC(=O)NC4=O)C5=O)CC3)cc2)CC1] | (E)-N-(4-(1-(4-(4-(7- ((2-(2,6- dioxopiperidin- 3-yl)-1- oxoisoindolin-4- yl)oxy)heptyl) piperazin- 1-yl)benzoyl). piperidin- 4-yl)butyl)- 3-(pyridin-3- yl)acrylamide | (E)-N-(4-(1-(4-(4-(7- ((2-(2,6- dioxopiperidin- 3-yl)-1- oxoisoindolin-4- yl)oxy)heptyl) piperazin-1- yl)benzoyl)piperidin- 4-yl)butyl)- 3-(pyridin-3- yl)acrylamide |
| SIAI S633 093 | 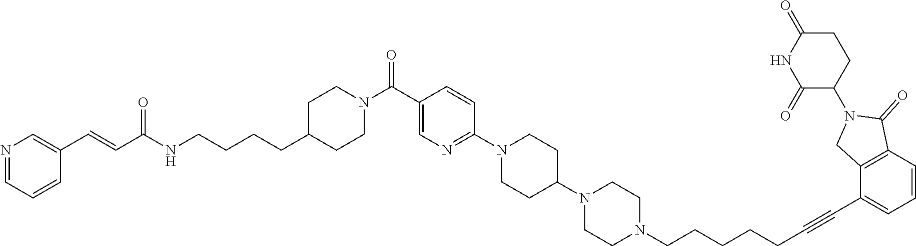 [SMILES: O=C(/C=C/c1cccnc1)NCCCCC1CCN(C(=O)c2ccc(N3CCC(N4CCN(CCCCCC#Cc5cccc6c5CN(C5CCC(=O)NC5=O)C6=O)CC4)CC3)nc2)CC1] | (E)-N-(4-(1-(6-(4-(4-(7- (2-(2,6- dioxopiperidin- 3-yl)-1- oxoisoindolin-4- yl)hept-6-yn-1- yl)piperazin-1- yl)piperidin-1- yl)nicotinoyl) piperidin- 4-yl)butyl)- 3-(pyridin-3- yl)acrylamide | (E)-N-(4-(1-(6-(4-(4- (7-(2-(2,6- dioxopiperidin- 3-yl)-1- oxoisoindolin-4- yl)hept-6-yn-1- yl)piperazin-1- yl)piperidin-1- yl)nicotinoyl) piperidin- 4-yl)butyl)- 3-(pyridin- 3-yl)acrylamide |
| SIAI S633 094 | 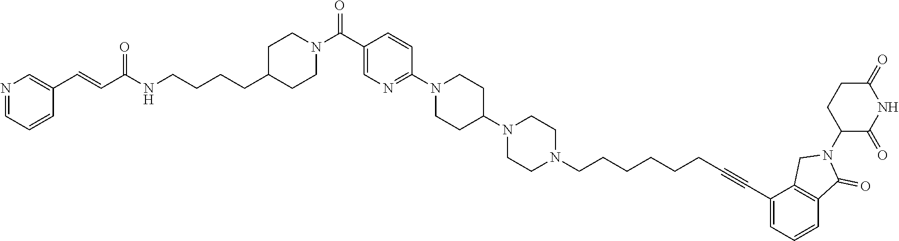 [SMILES: O=C(/C=C/c1cccnc1)NCCCCC1CCN(C(=O)c2ccc(N3CCC(N4CCN(CCCCCCC#Cc5cccc6c5CN(C5CCC(=O)NC5=O)C6=O)CC4)CC3)nc2)CC1] | (E)-N-(4-(1-(6-(4-(4-(8- (2-(2,6-dioxopiperidin- 3-yl)-1-oxoisoindolin- 4-yl)oct-7-yn-1- yl)piperazin-1- yl)piperidin-1- yl)nicotinoyl) piperidin- 4-yl)butyl)- 3-(pyridin-3- yl)acrylamide | (E)-N-(4-(1-(6-(4-(4- (8-(2-(2,6- dioxopiperidin- 3-yl)-1- oxoisoindolin-4- yl)oct-7-yn-1- yl)piperazin-1- yl)piperidin-1- yl)nicotinoyl) piperidin- 4-yl)butyl)- 3-(pyridin- 3-yl)acrylamide |
| SIAI S633 063 | 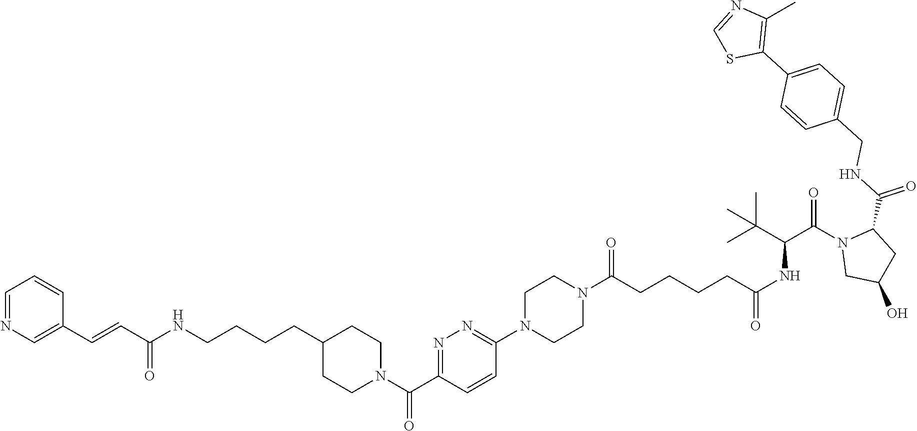 [SMILES: Cc1ncsc1-c1ccc(CNC(=O)[C@@H]2C[C@@H](O)CN2C(=O)[C@@H](NC(=O)CCCCC(=O)N2CCN(c3ccc(C(=O)N4CCC(CCCCNC(=O)/C=C/c5cccnc5)CC4)nn3)CC2)C(C)(C)C)cc1] | (2S,4R)-1-((S)-3,3- dimethyl-2-(6- oxo-6-(4- (1-(6-(4-(4-((E)-3- (pyridin-3- yl)acrylamido)butyl) piperidine-1- carbonyl)pyridazin- 3-yl)piperidin-4- yl)piperazin-1- yl)hexanamido) butanoyl)-4-hydroxy- N-(4-(4- methylthiazol-5- yl)benzyl) pyrrolidine-2- carboxamide | (2S,4R)-1-((S)-3,3- dimethyl-2- (6-oxo-6- (4-(1-(6-(4-(4- ((E)-3-(pyridin-3- yl)acrylamido) butyl)piperidine- 1-carbonyl) pyridazin-3- yl)piperidin-4- yl)piperazin-1- yl)hexanamido) butanoyl)-4- hydroxy-N-(4-(4- methylthiazol-5- yl)benzyl) pyrrolidine- 2-carboxamide |

### II. Other Forms of Compounds (Including Salts, Enantiomers, Stereoisomers, Solvates, Isotopically Enriched Analogs, Prodrugs, or Polymorphs of Compounds)

The compounds of the present disclosure have the structures of any one of Formula (I), Formula (II), Formula (V), Formula (VI), Formula (VII), or Formula (VIII). Unless otherwise specified, all references to the compound of the present disclosure also include compounds of any one of Formula (I), Formula (II), Formula (V), Formula (VI), Formula (VII), or Formula (VIII) and specific compounds within the scope of these general formulae.

It should be recognized that compounds of the present disclosure (including Formula (I), Formula (II), Formula (V), Formula (VI), Formula (VII), and Formula (VIII)) may have a stereo-configuration and thus can exist in more than one stereoisomeric form. The present disclosure also relates to optically enriched compounds having a stereo-configuration, e.g., greater than about 90% enantiomeric/diastereomeric excess (“ee”), such as about 95% ee or 97% ee, or greater than 99% ee, and mixtures thereof, including racemic mixtures. As used herein, “optically enriched” means that a mixture of enantiomers consists of a significantly greater proportion of one enantiomer, and can be described by enantiomeric excess (ee %). Purification of isomers and separation of mixtures of isomers can be accomplished by standard techniques known in the art (e.g., column chromatography, preparative TLC, preparative HPLC, asymmetric synthesis (e.g., by using chiral intermediates) and/or or chiral resolution, etc.).

In some embodiments, polymorph forms or salts of the compounds of the present disclosure are also provided. Salts of the compounds of the present disclosure can be pharmaceutically acceptable salts including, but not limited to, hydrochloride, sulfate, citrate, maleate, sulfonate, citrate, lactate, tartrate, fumarate, phosphate, dihydrogenphosphate, pyrophosphate, metaphosphate, oxalate, malonate, benzoate, mandelate, succinate, trifluoroacetate, glycolate, or p-toluenesulfonate, etc. The compounds of the present disclosure can exist as non-solvated or solvated forms in pharmaceutically acceptable solvents such as water, ethanol, and the like. In some embodiments, compounds of the present disclosure can be prepared as prodrugs or precursor drugs. Prodrugs can be converted into parent drugs in the body to play their role. In some embodiments, isotopically-labeled compounds of the present disclosure are also provided, examples of which include deuterium (D or ²H).

### III. Compositions/Formulations

In some embodiments, the present disclosure provides a pharmaceutical composition comprising as active ingredient the compound of the present disclosure or a pharmaceutically acceptable salt, solvate, isotopically enriched analog, polymorph, prodrug, stereoisomer (including enantiomer), or mixture of stereoisomers thereof, and at least one pharmaceutically acceptable carrier.

In some embodiments, pharmaceutically acceptable carriers include, but are not limited to, fillers, stabilizers, dispersants, suspending agents, diluents, excipients, thickeners, colorants, solvents, or encapsulating materials. Each carrier must be “acceptable” in the sense of being compatible with the other ingredients of the formulation (including the compounds useful in the present disclosure) and not injurious to the patient. Some examples of materials that can be used as pharmaceutically acceptable carriers include: sugars such as lactose, glucose, and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository wax; oils such as peanut oil, cottonseed oil, safflower oil, sesame oil, olive oil, corn oil and soybean oil; glycols such as propylene glycol; polyols such as glycerol, sorbitol, mannitol, and polyethylene glycol; esters such as ethyl oleate and ethyl laurate; agar; buffers such as magnesium hydroxide and aluminum hydroxide; surfactant phosphate buffer solution; polyethylene oxide, polyvinylpyrrolidone, polyacrylamide, poloxamer; and other common non-toxic compatible substances used in pharmaceutical formulations.

The pharmaceutical composition of the present disclosure can further comprise at least one second therapeutic agent for treating or preventing diseases or disorders associated with nicotinamide phosphoribosyltransferase (NAMPT). The second therapeutic agent may be used in combination with the compound of Formula (I) described in the present disclosure to treat the diseases or disorders associated with NAMPT. The second therapeutic agent includes, but is not limited to, chemotherapeutic agents, immunotherapeutic agents, gene therapy agents, and the like. In some embodiments, the diseases or disorders associated with NAMPT include tumors, autoimmune diseases, inflammatory diseases, gestational hypertension, cardiovascular and cerebrovascular diseases, obesity and diabetic nephropathy.

In some embodiments, the diseases or disorders associated with NAMPT comprises: colorectal cancer; breast cancer (including triple negative breast cancer, invasive breast cancer, infiltrating breast cancer); astrocytoma; pancreatic cancer; gastric cancer; prostate cancer; melanoma; leukemia (such as acute myeloid leukemia, acute lymphocytic leukemia); ovarian cancer; liver cancer; lung cancer (such as non-small cell lung cancer and small cell lung cancer); glioblastoma; multiple myeloma; esophageal cancer; bladder cancer; thyroid cancer; endometrial cancer; lymphoma (such as diffuse large B-cell carcinoma, follicular B-cell lymphoma, Hodgkin's lymphoma, peripheral T-cell lymphoma); neuroendocrine tumors; renal cancer (such as renal oncocytoma, clear cell renal cell carcinoma, renal urothelial carcinoma); pediatric gliomas; rhabdomyosarcoma; leiomyosarcoma; urothelial carcinoma; basal cell carcinoma; oral squamous cell carcinoma; cholangiocarcinoma; bone cancer; cervical cancer; skin cancer; oral squamous cell carcinoma; autoimmune diseases (such as rheumatoid arthritis, autoimmune encephalitis); cardiovascular and cerebrovascular diseases (including coronary atherosclerosis, acute myocardial infarction, myocardial ischemia-reperfusion injury, ischemic stroke); inflammatory diseases; gestational hypertension; obesity; and diabetic nephropathy.

The pharmaceutical composition of the present invention comprising, as an active ingredient, the compound of Formula (I) of the present disclosure or a pharmaceutically acceptable salt thereof can be formulated into any suitable formulations such as sprays, patches, tablets (such as conventional tablets, dispersible tablets, orally disintegrating tablets), capsules (such as soft capsules, hard capsules, enteric-coated capsules), dragees, troches, powders, granules, powder injections, suppositories, or liquid formulations (such as suspensions (e.g., aqueous or oily suspensions), solutions, emulsions, or syrups), or conventional injection dosage forms such as injectable solutions (e.g., sterile injectable solutions formulated according to methods known in the art using water, Ringer's solution, or isotonic sodium chloride solution or the like as a vehicle or solvent) or lyophilized injectable formulation and the like, depending upon a suitable route of administration (including, but not limited to, nasal administration, inhalation administration, topical administration, oral administration, oral mucosal administration, rectal administration, intrapleural administration, intraperitoneal administration, vaginal administration, intramuscular administration, subcutaneous administration, transdermal administration, epidural administration, intrathecal administration, and intravenous administration). Those skilled in the art can also formulate the compound of formula (I) of the present disclosure into conventional, dispersible, chewable, orally disintegrating or rapidly dissolving formulations, or sustained-release capsules or controlled-release capsules as needed.

### IV. Kits/Packaged Products

The compound of Formula (I) or a pharmaceutically acceptable salt, solvate, isotopically enriched analog, polymorph, prodrug, stereoisomer (including enantiomer), or mixture of stereoisomers thereof is used as a medicament. In some embodiments, the medicament of the present disclosure or the composition of the present disclosure may be presented in a kit/packaged product. The kit/packaged product may include a package or container including, but not limited to, ampoules, blister packs, pharmaceutical plastic bottles, vials, pharmaceutical glass bottles, containers, syringes, laminated flexible packaging, co-extruded film infusion containers, test tubes and dispensing devices, and the like. The kit/packaged product may contain instructions for use of the product.

### V. Methods and Uses

The compound of Formula (I) or a pharmaceutically acceptable salt, solvate, isotopically enriched analog, polymorph, prodrug, stereoisomer (including enantiomer), or mixture of stereoisomers thereof can be used as a medicament. Especially the compound of Formula (I) or a pharmaceutically acceptable salt, solvate, isotopically enriched analog, polymorph, prodrug, stereoisomer (including enantiomer), or mixture of stereoisomers thereof can be used for the manufacture of a medicament for the prevention and/or treatment of a disease or disorder associated with NAMPT.

The present disclosure provides a method for treating or preventing a disease or disorder associated with NAMPT in a subject, comprising administering to the subject a therapeutically effective amount of the compound of Formula (I) or a pharmaceutically acceptable salt, or the pharmaceutical composition of the present disclosure. In some embodiments, the disease or disorder associated with NAMPT comprises: tumors, autoimmune diseases, inflammatory diseases, gestational hypertension, cardiovascular and cerebrovascular diseases, obesity and diabetic nephropathy. In some embodiments, the disease or disorder associated with NAMPT comprises: colorectal cancer; breast cancer (including triple negative breast cancer, invasive breast cancer, infiltrating breast cancer); astrocytoma; pancreatic cancer; gastric cancer; prostate cancer; melanoma; leukemia (such as acute myeloid leukemia, acute lymphocytic leukemia); ovarian cancer; liver cancer; lung cancer (such as non-small cell lung cancer and small cell lung cancer); glioblastoma; multiple myeloma; esophageal cancer; bladder cancer; thyroid cancer; endometrial cancer; lymphoma (such as diffuse large B-cell carcinoma, follicular B-cell lymphoma, Hodgkin's lymphoma, peripheral T-cell lymphoma); neuroendocrine tumors; renal cancer (such as renal oncocytoma, clear cell renal cell carcinoma, renal urothelial carcinoma); pediatric gliomas; rhabdomyosarcoma; leiomyosarcoma; urothelial carcinoma; basal cell carcinoma; oral squamous cell carcinoma; cholangiocarcinoma; bone cancer; cervical cancer; skin cancer; oral squamous cell carcinoma; autoimmune diseases (such as rheumatoid arthritis, autoimmune encephalitis); cardiovascular and cerebrovascular diseases (including coronary atherosclerosis, acute myocardial infarction, myocardial ischemia-reperfusion injury, ischemic stroke); inflammatory diseases; gestational hypertension; obesity; and diabetic nephropathy.

In the method for treating or preventing a disease or disorder associated with NAMPT in a subject, the compound of Formula (I) or the pharmaceutical composition of the present disclosure is administered to the subject through at least one mode of administration selected from the group consisting of nasal administration, inhalation administration, topical administration, oral administration, oral mucosal administration, rectal administration, pleural cavity administration, peritoneal administration, vaginal administration, intramuscular administration, subcutaneous, transdermal, epidural, intrathecal, and intravenous administration.

The term “treatment” or “treating” refers to the administration of the compound of Formula (I) or a pharmaceutically acceptable salt thereof according to the present disclosure, or the pharmaceutical composition containing, as an active ingredient, the compound of Formula I or a pharmaceutically acceptable salt thereof, to a subject to mitigate (alleviate) undesirable diseases or conditions, such as the development of a cancer or tumor. The beneficial or desired clinical results of the present disclosure include, but are not limited to: alleviating symptoms, reducing the severity of the disease, stabilizing the state of the disease, slowing down or delaying the progression of the disease, improving or alleviating the condition, and alleviating the disease.

A “therapeutic effective amount” of the compound of the present disclosure depends on a variety of factors, including the activity of the specific compound used, the metabolic stability of the compound and the duration of its action, the age, sex and weight of the patient, the patient's current medical condition, the route and duration of administration, the excretion rate, the combined administration of additional drugs, and the progression of the diseases or conditions of the patient being treated. Those skilled in the art will be able to determine appropriate dosages based on these and other factors.

It is to be understood that the choice of using one or more active compounds and/or compositions and their dosage depends on the basic situations of the individual (which should generally render the individual situation to achieve the best effect). Dosing and dosing regimens should be within the ability of those skilled in the art, and the appropriate dosage depends on many factors including the knowledge and ability of the physicians, veterinarians or researchers (Jun Li (chief editor), “Clinical Pharmacology”, 4th edition, People's Public Health Press, 2008).

As used herein, the term “patient” or “subject” refers to animal, for example mammal, including but not limited to primate (such as human being), cow, sheep, goat, horse, dog, cat, rabbit, guinea pig, rat, mice, etc.

### VI. Definitions

Unless otherwise specified, the following words, phrases and symbols used herein generally have the meanings as described below.

In general, the nomenclature used herein (including the IUPAC nomenclature) and the laboratory procedures described below (including those used in cell culture, organic chemistry, analytical chemistry, and pharmacology, etc.) are those well-known and commonly used in the art. Unless otherwise defined, all scientific and technical terms used herein in connection with the present disclosure described herein have the same meaning as commonly understood by one skill in the art. In addition, the use of the word “a” or “an” when used in conjunction with the term “comprising” or a noun in the claims and/or the specification may mean “one”, but it is also consistent with the meaning of “one or more”, “at least one”, and “one or more than one”. Similarly, the terms “another” or “other” can mean at least a second or more.

It should be understood that whenever the term “comprise” or “include” is used herein to describe various aspects, other similar aspects described by “consisting of” and/or “consisting essentially of” are also provided.

As used herein, the term “about” used alone or in combination refers to approximately, roughly, nearly, or around. When the term “about” is used in conjunction with a numerical range, it modifies that range by extending the boundaries above and below the stated numerical value. In general, the term “about” can modify a numerical value above and below the stated value by an upward or downward (increasing or decreasing) variation, e.g., 10%, 5%, 2%, or 1%.

As used herein, the wording “ . . . represents a bond” used alone or in combination means that the referenced group is a bond linker (that is, the referenced group is absent). For example, the wording “R represents a bond” means that R is a bond linker. In other words, when R presents a bond, the group W in the structure of Formula (III) is directly connected to phenyl ring in the structure of Formula (III).

As used herein, the term “inserted” of the expression “one or more groups R⁵ and/or one or more groups R⁶ and/or any combination of one or more groups R⁵ and R⁶ are inserted into the backbone carbon chain of the linear or branched C₂₋₆₀ alkylene group”, used alone or in combination, has a known definition in the art, which can mean that carbon-carbon bond between one or more pairs of adjacent carbon atoms in the referenced backbone carbon chain is interrupted by the groups R⁵, R⁶, or a combination of R⁵ and R⁶. Herein, non-limiting examples of the above-mentioned expression “one or more groups . . . are inserted into” may include, but are not limited to, that one or more (e.g., 1-30, 1-20, or 1-15, 1-10, 1-9, 1-8, 1-7, 1-6, 1-5, 1-4, 1-3, or 1-2, or 1) groups R⁵ as defined herein and/or one or more (e.g., 1-30, 1-20, or 1-15, 1-10, 1-9, 1-8, 1-7, 1-6, 1-5, 1-4, 1-3, or 1-2, or 1) groups R⁶ as defined herein and/or one or more (e.g., 1-30, 1-20, or 1-15, 1-10, 1-9, 1-8, 1-7, 1-6, 1-5, 1-4, 1-3, or 1-2 ↑, or 1) combinations of R⁵ with R⁶ are inserted into the backbone carbon chain, and the resulting backbone chain group conforms to the covalent bond theory. For example, the expression “one or more groups R⁵ and/or one or more groups R⁶ and/or any combination of one or more groups R⁵ and R⁶ are inserted into the backbone carbon chain of the linear or branched C₂₋₆₀ alkylene group” can refer to that one or more (e.g., 1-30, 1-20, 1-15, 1-10, 1-8, 1-7, 1-6, 1-5, 1-4, 1-3, 1-2, or 1) groups R⁵ and/or R⁶ and/or one or more combinations of R⁵ with R⁶ are inserted between one or more pairs of any two adjacent carbon atoms of the backbone carbon chain of the linear or branched C₂₋₆₀ alkylene group, resulting in the formation of one or more (e.g., 1-30, 1-20, 1-15, 1-10, 1-8, 1-7, 1-6, 1-5, 1-4, 1-3, 1-2, or 1) fragments “—CH₂—R⁵—CH₂—” and/or one or more (e.g., 1-30, 1-20, 1-15, 1-10, 1-8, 1-7, 1-6, 1-5, 1-4, 1-3, 1-2, or 1) fragments “—CH₂—R⁶—CH₂—” and/or one or more (e.g., 1-30, 1-20, 1-15, 1-10, 1-8, 1-7, 1-6, 1-5, 1-4, 1-3, 1-2, or 1) fragments “—CH₂—R⁵—R⁶—CH₂—”, where each R⁵ are the same or different, each R⁶ are the same or different, and are as defined herein.

Herein, it should be understood that the expression “one or more groups R⁵ and/or one or more groups R⁶ and/or any combination of one or more groups R⁵ and R⁶ are optionally inserted into the backbone carbon chain of the linear or branched C₂₋₆₀ alkylene group” includes embodiments where “one or more groups R⁵ and/or one or more groups R⁶ and/or any combination of one or more groups R⁵ and R⁶ are inserted into the backbone carbon chain of the linear or branched C₂₋₆₀ alkylene group”, as well as embodiments where “no one or more groups R⁵ and/or one or more groups R⁶ and/or any combination of one or more groups R⁵ and R⁶ are optionally inserted into the backbone carbon chain of the linear or branched C₂₋₆₀ alkylene group”.

Herein, a bond interrupted by a wavy line shows the point of attachment of the depicted group to the rest of the molecule. For example, the group of ULM depicted below

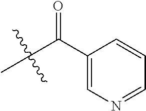 [SMILES: *C(=O)c1cccnc1]

indicates that Z₁ of the referenced group is connected to the alkylene of LIN of the compound of Formula (I).

As used herein, the expression “a hydrogen atom of one or more CH₂ of the linear or branched C_{x-y} alkylene is replaced by . . . ”, used alone or in combination, means that a hydrogen atom of any one or more CH₂ of the linear or branched C_{x-y} alkylene is replaced by a substituent(s) as defined herein. Herein, the term “one or more” of “a hydrogen atom of one or more CH₂ of groups #-U—CH₂—, #-U—(CH₂)₂—, #-U—(CH₂)₃—, #-U—(CH₂)₄—, #-U—(CH₂)₅—, #-U—(CH₂)₆—, #-U—(CH₂)₇—, #-U—(CH₂)₈—, #-U—(CH₂)₉—, #-U—(CH₂)₁₀—, #-U—(CH₂)₁₁—, #-U—(CH₂)₁₂—, #-U—(CH₂)₁₃—, #-U—(CH₂)₁₄—, #-U—(CH₂)₁₅—, #-U—(CH₂)₁₆—, #-U—(CH₂)₁₇—, #-U—(CH₂)₁₈—, #-U—(CH₂)₁₉—, #-U—(CH₂)₂₀—, #-U—(CH₂)₂₁—, #-U—(CH₂)₂₂—, #-U—(CH₂)₂₅—, #-U—(CH₂)₃₀—, #-U—(CH₂)₃₅—, #-U—(CH₂)₄₀—, #-U—(CH₂)₄₅—, #-U—(CH₂)₅₀—, #-U—(CH₂)₅₅—, or #-U—(CH₂)₆₀—” may include part or all hydrogen atoms of each referenced alkylene group, including but not limited to 1-80 hydrogens. In some embodiments, the expression “a hydrogen atom of one or more CH₂” may refer to part or all of the hydrogen atoms of the referenced alkylene group, including but not limited to 1-30, such as 1-25, 1-20, 1-15, 1-10, 1-5, 1-4, 1-3, 1-2 or 1 hydrogen atoms. In some embodiments, the expression “a hydrogen atom of one or more CH₂” may include 1-3 of the plurality of hydrogen atoms of the referenced alkylene group. The number of hydrogens to be replaced is in principle not limited in any way, or is automatically limited by the size of the building unit.

As used herein, the wordings “optionally substituted” and “unsubstituted or substituted” can be used interchangeably. The term “substituted” usually means that one or more hydrogen atoms in the structure referenced are replaced by the same or different specific substituents.

As used herein, the term “oxo” or “oxo group” refers to ═O.

As used herein, the term “C(O)” or “C(═O)” or “C═O” used alone or in combination refers to a carbonyl group.

As used herein, the term “nicotinoyl” refers

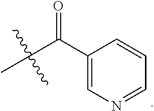 [SMILES: *C(=O)c1cccnc1]

As used herein, the term “halogen atom” or “halogen”, used alone or in combination, refers to fluorine, chlorine, bromine, or iodine.

As used herein, the term “alkyl”, used alone or in combination, refers to a linear or branched alkyl group. The term “Cₓ-C_{y} alkyl” or “C_{x-y} alkyl” (x and y each being an integer) refers to a linear or branched alkyl group containing from x to y carbon atoms. The term “C₁₋₁₀ alkyl” used alone or in combination in the present disclosure refers to a linear or branched alkyl group containing from 1 to 10 carbon atoms. Non-limiting examples of the C₁₋₁₀ alkyl of the present disclosure may include a C₁₋₉ alkyl, C₁₋₈ alkyl, C₂₋₈ alkyl, C₁₋₇ alkyl, C₁₋₆ alkyl, C₁₋₅ alkyl, and C₁₋₄ alkyl. Representative examples include methyl, ethyl, n-propyl, isopropyl, n-butyl, isobutyl, sec-butyl, tert-butyl, pentyl, isopentyl, neopentyl, tert-pentyl, hexyl, heptyl, octyl, nonyl, and decyl. The term “C₁₋₃ alkyl” in the present disclosure refers to an alkyl group containing from 1 to 3 carbon atoms, and representative examples thereof include methyl, ethyl, n-propyl, and isopropyl. In the present disclosure, the “alkyl” is optionally substituted by one or more selected from the group consisting of halogen, hydroxyl, cyano, C₁₋₃ alkyl, C₁₋₃ alkoxy, trifluoromethyl, heterocyclyl, or a combination thereof.

As used herein, the term “halogenated alkyl” or “haloalkyl”, used alone or in combination, refers to a linear or branched alkyl group substituted with one or more halogens, wherein one or more hydrogen atom(s) of the alkyl group is replaced with one or more halogens. The term “halogenated C_{x-Cy} alkyl” or “halogenated C_{x-y} alkyl” (x and y are each an integer) refers to a linear or branched alkyl containing from x to y carbon atoms substituted with one or more halogens. The term “halogenated C₁₋₁₀ alkyl” used alone or in combination in the present invention refers to a linear or branched alkyl group containing from 1 to 10 carbon atoms substituted with one or more halogens. Examples of the halogenated C₁₋₁₀ alkyl group of the present disclosure include halogenated C₁₋₉ alkyl group, e.g., halogenated C₁₋₈ alkyl group, halogenated C₂₋₈ alkyl group, C₁₋₇ alkyl group, halogenated C₁₋₆ alkyl, halogenated C₁₋₅ alkyl, or halogenated C₁₋₄ alkyl. Representative examples include halomethyl, haloethyl, halo-n-propyl, haloisopropyl, halo-n-butyl, haloisobutyl, halo-sec-butyl, halo-tert-butyl, halopentyl, haloisoamyl, haloneopentyl, halo-tert-pentyl, halohexyl, haloheptyl, halooctyl, halononyl, and halodecyl. The term “halo-C₁₋₃ alkyl” of the present disclosure refers to an alkyl group containing from 1 to 3 carbon atoms substituted by one or more halogens, and its representative examples include halomethyl, haloethyl, halo-n-propyl and haloisopropyl.

As used herein, the term “alkylene” (which is used interchangeably with “alkylene chain”), used alone or in combination, refers to a linear or branched divalent saturated hydrocarbon group composed of carbon and hydrogen atoms. The term “Cₓ-C_{y} alkylene” or “C_{x-y} alkylene” (x and y each being an integer) refers to a linear or branched alkylene group containing from x to y carbon atoms. Examples of the C₁-C₆₀ alkylene in the present disclosure may include C₁-C₅₅ alkylene, C₁-C₅₀ alkylene, C₁-C₄₅ alkylene, C₁-C₄₀ alkylene, C₁-C₃₅ alkylene, C₁-C₃₀ alkylene, C₁-C₂₉ alkylene, C₁-C₂₈ alkylene, C₁-C₂₇ alkylene, C₁-C₂₆ alkylene, C₁-C₂₅ alkylene, C₁-C₂₄ alkylene, C₁-C₂₃ alkylene, C₁-C₂₂ alkylene, C₁-C₂₁ alkylene, C₁-C₂₀ alkylene, C₁-C₁₉ alkylene, C₁-C₁₈ alkylene, C₁-C₁₇ alkylene, C₁-C₁₆ alkylene, C₁-C₁₅ alkylene, C₁-C₁₄ alkylene, C₁-C₁₃ alkylene, C₁-C₁₂ alkylene, C₁-C₁₁ alkylene, C₁-C₁₀ alkylene, C₁-C₉ alkylene, C₁-C₈ alkylene, C₁-C₇ alkylene, C₁-C₆ alkylene, C₁-C₅ alkylene, C₁-C₄ alkylene, C₁-C₃ alkylene, or C₁-C₂ alkylene. Representative examples include, but are not limited to, methylene, ethylene, propylene, isopropylene, butylene, isobutylene, sec-butylene, tert-butylene, n-pentylene, isopentylene, neopentylidene, tert-pentylene, hexylene, heptylene, octylene, nonylene, decylene, undecylene, dodecylene, tridecylene, tetradecylene, pentadecylene, hexadecylene, heptadecylene, octadecylene, nonadecylene, eicosylene, heneicosylene, docosylene, tricosylene, tetracosylene, pentacosylene, hexacosylene, peptacosylene, octacosylene, nonacosylene, and triacontylene. In the present disclosure, the “alkylene” is optionally substituted by one or more substituents selected from C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “alkoxy”, used alone or in combination, refers to a linear or branched alkoxy group having structural formula of —O-alkyl. Optionally, the alkyl portion of the alkoxy group may contain 1-10 carbon atoms. Representative examples of “alkoxy” include, but are not limited to, methoxy, ethoxy, propoxy, isopropoxy, n-butoxy, isobutoxy, tert-butoxy, pentyloxy, 2-pentyloxy, isopentyloxy, neopentyloxy, hexyloxy, 2-hexyloxy, 3-hexyloxy, 3-methylpentyloxy, etc. The term “C₁-C₃ alkoxy” or “C₁₋₃ alkoxy” refers to a linear or branched alkoxy group containing from 1 to 3 carbon atoms. Representative examples of C₁₋₃ alkoxy include, but are not limited to, methoxy, ethoxy, n-propoxy, and isopropoxy. Representative examples of C₁₋₃ alkoxy include, but are not limited to, methoxy, ethoxy, n-propoxy, and isopropoxy.

As used herein, the term “alkylamino”, used alone or in combination, refers to a linear or branched alkylamino group having structural formula of alkyl-NH—. Optionally, the alkyl portion of the alkylamino group may contain 1-10 carbon atoms. Representative examples of “alkylamino” include, but are not limited to, methyl-NH—, ethyl-NH—, n-propyl-NH—, isopropyl-NH—, n-butyl-NH—, isobutyl-NH—, tert-butyl-NH—, pentyl-NH—, and hexyl-NH—, etc. The term “C₁-C₃ alkyl-NH—” or “C₁₋₃ alkyl-NH—” refers to a linear or branched alkyl-NH— group containing from 1 to 3 carbon atoms. Representative examples of C₁₋₃ alkyl-NH— include, but are not limited to, methyl-NH—, ethyl-NH—, n-propyl-NH—, and isopropyl-NH—.

As used herein, the term “amino-substituted alkylene”, used alone or in combination, refers to a linear or branched alkylene group substituted by amino having structural formula of NH₂-alkylene. Optionally, the alkylene portion of the amino-substituted alkylene group may contain 1-10 carbon atoms. The term “amino-substituted C₁₋₃ alkylene” or “NH₂—C₁₋₃ alkyl” refers to a linear or branched alkylene group substituted by amino containing from 1 to 3 carbon atoms. Representative examples of amino-substituted C₁₋₃ alkylene include, but are not limited to, NH₂—CH₂—, NH₂—CH₂CH₂—, and NH₂—CH₂CH₂CH₂—.

As used herein, the term “alkyl-NHC(O)—”, used alone or in combination, refers to a linear or branched alkyl-NHC(O)— group having structural formula of alkyl-NHC(O)—. Optionally, the alkyl portion of the alkyl-NHC(O)— group may contain 1-10 carbon atoms. The term “C₁₋₃ alkyl-NHC(O)—” or “C₁-C₃ alkyl-NHC(O)—” refers to a linear or branched alkyl-NHC(O)— group containing from 1 to 3 carbon atoms. Representative examples of C₁₋₃ alkyl-NHC(O)— include, but are not limited to, CH₃—NHC(O)—, CH₃CH₂—NHC(O)—, and CH₃CH₂CH₂—NHC(O)—.

As used herein, the term “alkyl-C(O)NH—”, used alone or in combination, refers to a linear or branched alkyl-C(O)NH— group having structural formula of alkyl-C(O)NH—. Optionally, the alkyl portion of the alkyl-C(O)NH— group may contain 1-10 carbon atoms. The term “C₁₋₃ alkyl-C(O)NH—” or “C₁-C₃ alkyl-C(O)NH—” refers to a linear or branched alkyl-C(O)NH— group containing from 1 to 3 carbon atoms. Representative examples of C₁₋₃ alkyl-C(O)NH— include, but are not limited to, CH₃—C(O)NH—, CH₃CH₂—C(O)NH—, and CH₃CH₂CH₂—C(O)NH—.

As used herein, the term “heteroaryl”, used alone or in combination, refers to a 5- to 20-membered (e.g., 5- to 15-membered, 5- to 12-membered, 5- to 11-membered, 5- to 10-membered, 5- to 9-membered, 5- to 8-membered, 5- to 7-membered, 5- to 6-membered, 6- to 15-membered, or 6- to 9-membered) monocyclic, bicyclic, or polycyclic aromatic ring group containing at least one aromatic ring having one or more (e.g., from 1 to 6, or from 1 to 5, or from 1 to 4, or from 1 to 3) heteroatoms independently selected from the group consisting of oxygen, nitrogen, and sulfur. Bicyclic or polycyclic heteroaryl groups include bicyclic, tricyclic or tetracyclic heteroaryl groups, which contain one aromatic ring having one or more heteroatoms independently selected from O, S and N, and the remaining rings may be a saturated, partially unsaturated or aromatic ring and can be carbocyclic ring or contain one or more heteroatoms independently selected from O, S and N. Examples of monocyclic heteroaryl groups include, but are not limited to, furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, tetrazolyl, and triazinyl. Examples of bicyclic heteroaryl groups include, but are not limited to, indolyl, isoindolyl, isoindolinyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, oxazolopyridyl, furopyridyl, pteridyl, purinyl, pyridopyridyl, pyrazolo[1,5-a]pyridyl, pyrazolo[1,5-a]pyrimidinyl, imidazo[1,2a]pyridyl, 1H-pyrrolo[3,2-b]pyridyl, 1H-pyrrolo[2,3-b]pyridyl, pyrrolo[2,1-b]thiazolyl and imidazo[2,1-b]thiazolyl. Examples of tricyclic heteroaryl groups include, but are not limited to, acridinyl, benzindolyl, carbazolyl, dibenzofuranyl, and xanthyl. The heteroaryl group may be unsubstituted or substituted. A substituted heteroaryl refers to heteroaryl substituted one or more times (e.g., 1-4, 1-3, or 1-2 times) by a substituent(s) optionally selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “heteroarylene”, used alone or in combination, refers to a 5- to 20-membered (e.g., 5- to 15-membered, 5- to 12-membered, 5- to 11-membered, 5- to 10-membered, 5- to 9-membered, 5- to 8-membered, 5- to 7-membered, 5- to 6-membered, 6- to 15-membered, or 6- to 9-membered) monocyclic, bicyclic, or polycyclic bivalent aromatic ring group containing at least one aromatic ring having one or more (e.g., from 1 to 6, or from 1 to 5, or from 1 to 4, or from 1 to 3) heteroatoms independently selected from the group consisting of oxygen, nitrogen, and sulfur. Bicyclic or polycyclic heteroarylene groups include bicyclic, tricyclic or tetracyclic heteroarylene groups, which contain one aromatic ring having one or more heteroatoms independently selected from O, S and N, and the remaining ring(s) may be a saturated, partially unsaturated or aromatic ring and can be carbocyclic ring or contain one or more heteroatoms independently selected from O, S and N. Examples of monocyclic heteroarylene groups include, but are not limited to, furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, tetrazolylene, and triazinylene. Examples of bicyclic heteroarylene groups include, but are not limited to, indolylene, isoindolylene, isoindolinylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, oxazolopyridylene, furopyridylene, pteridylene, purinylene, pyridopyridylene, pyrazolo[1,5-a]pyridylene, pyrazolo[1,5-a]pyrimidinylene, imidazo[1,2a]pyridylene, 1H-pyrrolo[3,2-b]pyridylene, 1H-pyrrolo[2,3-b]pyridylene, pyrrolo[2,1-b]thiazolylene, and imidazo[2,1-b]thiazolylene. Examples of tricyclic heteroarylene groups include, but are not limited to, acridinylene, benzindolylene, carbazolylene, dibenzofuranylene, and xanthylene. The heteroarylene group may be unsubstituted or substituted. A substituted heteroarylene refers to heteroarylene substituted one or more times (e.g., 1-4, 1-3, or 1-2 times) by a substituent(s) optionally selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “aryl”, used alone or in combination, refers to a monovalent aromatic hydrocarbon group containing from 5 to 14 carbon atoms and optionally one or more fused rings, such as phenyl group, naphthyl group, or fluorenyl group. As used herein, the “aryl” is optionally substituted. A substituted aryl group refers to an aryl group substituted one or more times (e.g., 1-4, 1-3, or 1-2 times) with a substituent(s). For example, aryl is mono-, di-, or tri-substituted by a substituent(s) optionally selected from e.g., C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “arylene”, used alone or in combination, refers to a divalent aromatic hydrocarbon group containing from 5 to 14 carbon atoms and optionally one or more fused rings, such as phenylene, naphthylene, or fluorenylene. As used herein, the “arylene” is optionally substituted. A substituted arylene refers to an arylene group optionally substituted one or more times (e.g., 1-4, 1-3, or 1-2 times) with a substituent(s). For example, arylene is mono-, di-, or tri-substituted by a substituent(s) optionally selected from e.g., C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “cycloalkyl”, used alone or in combination, refers to a saturated or partially unsaturated (i.e., containing one or more double bonds, but not having a fully conjugated π-electron system) monocyclic or bicyclic or polycyclic cyclic hydrocarbon radical, which in some embodiments contains from 3 to 20 carbon atoms (i.e., C₃₋₂₀ cycloalkyl), or from 3 to 15 carbon atoms (i.e., C₃₋₁₅ cycloalkyl), or from 3 to 12 carbon atoms (i.e., C₃₋₁₂ cycloalkyl), or from 3 to 11 carbon atoms (i.e., C₃₋₁₁ cycloalkyl), or from 3 to 10 carbon atoms (i.e., C₃₋₁₀ cycloalkyl), or from 3 to 8 carbon atoms (i.e., C₃₋₈ cycloalkyl), or from 3 to 7 carbon atoms (i.e., C₃₋₇ cycloalkyl), or from 3 to 6 carbon atoms (i.e., C₃₋₆ cycloalkyl). The term “cycloalkyl” includes monocyclic, bicyclic, or tricyclic cyclic hydrocarbon radical having from 3 to 20 carbon atoms. Representative examples of monocyclic cycloalkyl groups include, but are not limited to, cyclopropyl, cyclobutyl, cyclopentyl, cyclopentenyl, cyclohexyl, cyclohexenyl, cycloheptyl, and cyclooctyl. Bicyclic and tricyclic cycloalkyl groups include bridged cycloalkyl, fused cycloalkyl and spiro-cycloalkyl groups such as, but not limited to, decalinyl, octahydropentalenyl, octahydro-1H-indenyl, spiro-cycloalkyl, adamantanyl, noradamantanyl, bornyl, norbornyl (also named as bicyclo[2.2.1]heptyl by the IUPAC system). As used herein, the “cycloalkyl” is optionally mono- or poly-substituted, such as, but not limited to, 2,2-, 2,3-, 2,4-, 2,5-, or 2,6-disubstituted cyclohexyl. The substituents of the substituted “cycloalkyl” can be optionally one or more (e.g., 1-5, 1-4, 1-3, 1-2, or 1) substituents selected from C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof. Examples of “C₃₋₆ cycloalkyl” include, but are not limited to, cyclopropyl, cyclobutyl, cyclopentyl, cyclopentenyl, and cyclohexyl.

As used herein, the term “cycloalkylene”, used alone or in combination, refers to a saturated or partially unsaturated (i.e., containing one or more double bonds, but not having a fully conjugated π-electron system) monocyclic or bicyclic or polycyclic divalent cyclic hydrocarbon radical containing from 3 to 12 carbon atoms (e.g., 3-12, 3-11, 3-10, 3-8, 3-7, 3-6 carbon atoms). The term “cycloalkylene” includes monocyclic, bicyclic or tricyclic cycloalkylene having from 3 to 12 carbon atoms. Representative examples of monocyclic cycloalkylene groups include, but are not limited to, cyclopropylene, cyclobutylene, cyclopentylene, cyclopentenylene, cyclohexylene, cyclohexenylene, cycloheptylene, and cyclooctylene. Bicyclic and tricyclic cycloalkylene groups include bridged cycloalkylene, fused cycloalkylene and spiro-cycloalkylene groups such as, but not limited to, decalinylene, octahydropentalenylene, octahydro-1H-indenylene, 2,3-dihydro-1H-indenylene, spiro-cycloalkylene, adamantanylene, noradamantanylene, and norbornylene (also named as bicyclo[2.2.1]heptylene by the IUPAC system). As used herein, the “cycloalkylene” is optionally mono- or poly-substituted, such as, but not limited to, 2,2-, 2,3-, 2,4-, 2,5-, or 2,6-disubstituted cyclohexylene. The substituents of the substituted “cycloalkylene” can be optionally one or more substituents selected from C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “C_{x-y} spiro-cycloalkylene” (x and y each being an integer), used alone or in combination, refers to a spiro-cycloalkylene group containing from x to y carbon atoms. As used herein, the term “C₇₋₁₁ spiro-cycloalkylene”, used alone or in combination, refers to a spiro-cycloalkylene group containing from 7 to 11 carbon atoms (e.g., 7-10, 7-9 carbon atoms). Representative examples of “C₇₋₁₁ spiro-cycloalkylene” include, but are not limited to, spiro[3.3]heptylene, spiro[2.5]octylene, spiro[3.5]nonylene, spiro[4.4]nonylene, spiro[4.5]decylene, or spiro[5.5]undecylene. The “C₇₋₁₁ spiro-cycloalkylene” is optionally further substituted with one or more substituents selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “C_{x-y} spiro-heterocyclylene” (x and y each being an integer), used alone or in combination, refers to a spiro-heterocyclylene group containing from x to y carbon atoms and one or more (e.g., from 1 to 3, from 1 to 2, or 1) heteroatoms independently selected from sulfur, oxygen, and nitrogen. As used herein, the term “C₇₋₁₁ spiro-heterocyclylene”, used alone or in combination, refers to a spiro-heterocyclylene group containing from 7 to 11 carbon atoms (e.g., 7-10, or 7-9 carbon atoms) and one or more (e.g., from 1 to 3, from 1 to 2, or 1) heteroatoms independently selected from sulfur, oxygen, and nitrogen. Representative examples of “C₇₋₁₁ spiro-cycloalkylene” include (but are not limited to):

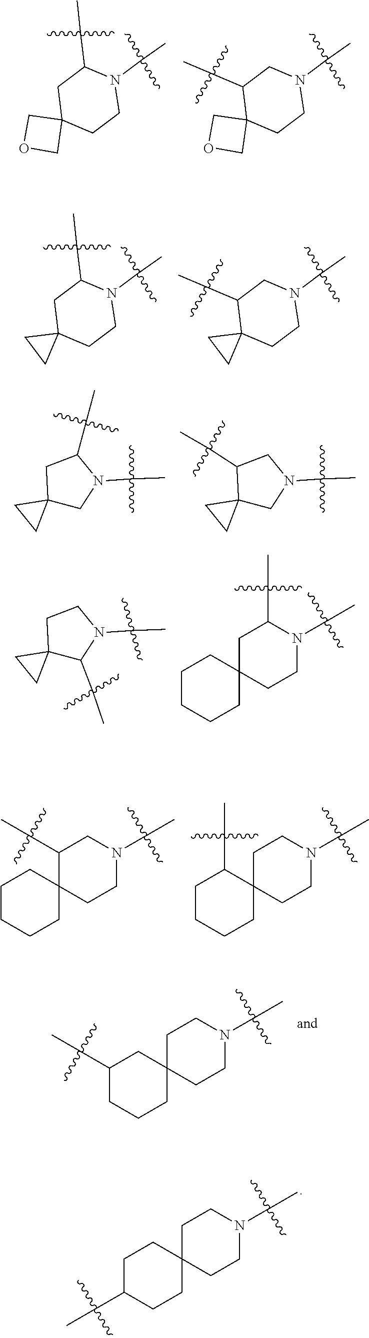

The “C₇₋₁₁ spiro-heterocyclylene” is optionally further substituted with one or more substituents selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “heterocyclyl” or “heterocyclic group”, used alone or in combination, refers to a 3- to 20-membered saturated or partially unsaturated (i.e., containing one or more double bonds, but not having a fully conjugated π-electron system) monocyclic, bicyclic, or tricyclic cyclic hydrocarbon group containing one or more (e.g., from 1 to 5, or from 1 to 4, from 1 to 3, from 1 to 2, or 1) heteroatoms independently selected from sulfur, oxygen, and nitrogen. In some embodiments, “heterocyclyl” may refer to a 3- to 15-membered (e.g., 3- to 14-membered, 3- to 12-membered, 3- to 11-membered, 3- to 10-membered, 3- to 9-membered, 3- to 8-membered, 3- to 7-membered, 3- to 6-membered, or 3- to 5-membered) saturated or partially unsaturated (i.e., containing one or more double bonds, but not having a fully conjugated π-electron system) monocyclic cyclic hydrocarbon group containing one or more (e.g., from 1 to 5, or from 1 to 4, from 1 to 3, from 1 to 2, or 1) heteroatoms independently selected from sulfur, oxygen, and nitrogen. Representative examples of the monocyclic heterocyclyl include, but are not limited to, azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, azacycloheptyl, azacyclooctyl, diazacycloheptanyl (e.g., 1,4-diazacycloheptan-1-yl), and diazacyclooctyl. Bicyclic and tricyclic heterocyclyl groups include bridged heterocyclyl, fused heterocyclyl and spiro-heterocyclyl groups such as, but not limited to, 6-azabicyclo[3.1.1]heptan-3-yl, 2,5-diazabicyclo[2.2.1]heptan-2-yl, 3,6-diazabicyclo[3.1.1]heptan-3-yl, 3-azabicyclo[3.2.1]octan-8-yl, 3,8-diazabicyclo[3.2.1]octan-8-yl, 3,8-diazabicyclo[3.2.1]octan-3-yl, 2,5-diazabicyclo[2.2.2]octan-2-yl, and azaspirocycloalkyl (including 3-azaspiro[5.5]undecan-3-yl). The heterocyclyl may be unsubstituted or substituted as explicitly defined (e.g., mono-, di-, tri-, or poly-substituted) by a substituent(s) optionally selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “heterocyclylene”, used alone or in combination, refers to a 3- to 20-membered saturated or partially unsaturated (i.e., containing one or more double bonds, but not having a fully conjugated π-electron system) monocyclic, bicyclic, or tricyclic bivalent cyclic hydrocarbon group containing one or more (e.g., from 1 to 5, or from 1 to 4, from 1 to 3, from 1 to 2, or 1) heteroatoms independently selected from sulfur, oxygen, and nitrogen. In some embodiments, “heterocyclylene” may refer to a 3- to 15-membered (e.g., 3- to 14-membered, 3- to 12-membered, 3- to 11-membered, 3- to 10-membered, 3- to 9-membered, 3- to 8-membered, 3- to 7-membered, 3- to 6-membered, or 3- to 5-membered) saturated or partially unsaturated (i.e., containing one or more double bonds, but not having a fully conjugated π-electron system) monocyclic cyclic hydrocarbon group containing one or more (e.g., from 1 to 5, or from 1 to 4, from 1 to 3, from 1 to 2, or 1) heteroatoms independently selected from sulfur, oxygen, and nitrogen. Representative examples of the monocyclic heterocyclylene include, but are not limited to, azetidinylene, oxetanylene, pyrrolidinylene, imidazolidinylene, pyrazolidylene, tetrahydrofuranylene, tetrahydropyranylene, tetrahydrothienylene, tetrahydrothiopyranylene, oxazolidinylene, thiazolidinylene, piperidinylene, piperazinylene, morpholinylene, thiomorpholinylene, dioxacyclohexylene, and diazacycloheptanylene (e.g., 1,4-diazacycloheptanylene, 4,5-diazacycloheptanylene, 1,3-diazacycloheptanylene). Bicyclic and tricyclic heterocyclylene groups include bridged heterocyclylene, fused heterocyclylene and spiro-heterocyclylene groups such as, but not limited to, 6-azabicyclo[3.1.1]heptanylene, 2,5-diazabicyclo[2.2.1]heptanylene, 3,6-diazabicyclo[3.1.1]heptanylene, 3-azabicyclo[3.2.1]octanylene, 3,8-diazabicyclo[3.2.1]octanylene, 3,8-diazabicyclo[3.2.1]octanylene, 2,5-diazabicyclo[2.2.2]octanylene, and azaspirocycloalkylene (e.g., 3-azaspiro[5.5]undecanylene). The heterocyclylene may be unsubstituted or substituted as explicitly defined (e.g., mono-, di-, tri-, or poly-substituted) by a substituent(s) optionally selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “alkynylene”, used alone or in combination, refers to a linear or branched divalent hydrocarbon group containing from 2 to 8 (e.g., from 2 to 6, from 2 to 5, from 2 to 4, or preferably 2) carbon atoms and having one or more (e.g., from 1 to 3, from 1 to 2, or 1) carbon-carbon triple bonds. Examples of alkynylene include, but are not limited to, ethynylene, 1-propynylene, 1-butynylene, and 1,3-diynylene.

As used herein, the term “alkenylene”, used alone or in combination, refers to a linear or branched divalent hydrocarbon group containing from 2 to 8 (e.g., from 2 to 6, from 2 to 5, from 2 to 4, from 2 to 3, or 2) carbon atoms and having one or more (e.g., from 1 to 3, from 1 to 2, or 1) carbon-carbon double bonds. Examples of alkenylene groups include, but are not limited to, vinylene (e.g., —CH═CH—), 1-propenylene, allylidene, 1-butenylene, 2-butenylene, 3-butenylene, isobutenylene, pentenylene, n-pent-2,4-dienylene, 1-methyl-but-1-enylene, 2-methyl-but-1-enylene, 3-methyl-but-1-enylene, 1-methyl-but-2-enylene, 2-methyl-but-2-enylene, 3-methyl-but-2-enylene, 1-methyl-but-3-enylene, 2-methyl-but-3-enylene, 3-methyl-but-3-enylene, and hexenylene.

As used herein, “bornylane” or “bornane” (also known as 1,7,7-trimethylbicyclo[2.2.1]heptane; camphane; bornylane) has a definition known to those skilled in the art. As used herein, “camphanyl” or “bornyl” refers to a monovalent group of bornane, i.e., the group remaining after any one of the hydrogens in bornane is removed. Representative examples of “bornyl” include, but are not limited to, 1,7,7-trimethylbicyclo[2.2.1]heptan-2-yl, 1,7,7-trimethylbicyclo[2.2.1]heptan-3-yl, 1,7,7-trimethylbicyclo[2.2.1]heptan-4-yl, 1,7,7-trimethylbicyclo[2.2.1]heptan-5-yl, 1,7,7-trimethylbicyclo[2.2.1]heptan-6-yl,

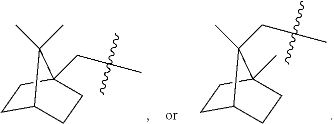 [SMILES: *CC12CCC(CC1)C2(C)C ; *CC1(C)C2CCC1(C)CC2]

As used herein, “bicyclo[2.2.1]heptane” also known as “norbornane”, has a definition known to those skilled in the art. As used herein, “bicyclo[2.2.1]heptyl” or “norbornyl” refers to a monovalent group of bicyclo[2.2.1]heptane, i.e., the group remaining after any hydrogen in bicyclo[2.2.1]heptane is removed. Representative examples of “bicyclo[2.2.1]heptyl” include, but are not limited to, bicyclo[2.2.1]heptan-2-yl, bicyclo[2.2.1]heptan-3-yl, bicyclo[2.2.1]heptan-4-yl, bicyclo[2.2.1]heptan-5-yl, or bicyclo[2.2.1]heptan-6-yl.

As used herein, the term “bicyclo[2.2.1]heptene” has a definition known to those skilled in the art. As used herein, “bicyclo[2.2.1]heptenyl” refers to a monovalent group of bicyclo[2.2.1]heptene, i.e., the group remaining after any hydrogen in bicyclo[2.2.1]heptene is removed. Representative examples of “bicyclo[2.2.1]heptenyl” include, but are not limited to, bicyclo[2.2.1]hept-5-en-2-yl, bicyclo[2.2.1]hept-5-en-3-yl, or bicyclo[2.2.1]hept-5-en-7-yl.

As used herein, “adamantane” (also known as tricyclo[3.3.1.1^{3,7}]decane) has a definition known to those skilled in the art, and its structural formula is e.g., as follows:

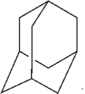

As used herein, “adamantanyl” refers to a monovalent group of adamantane, that is, the group remaining after any hydrogen in adamantane is removed. Representative examples of “adamantanyl” include, but are not limited to, 1-adamantanyl, 2-adamantanyl, 3-adamantanyl, 4-adamantanyl, 5-adamantanyl, 6-adamantanyl, 7-adamantanyl, 8-adamantanyl, 9-adamantanyl, or 10-adamantanyl.

As used herein, “noradamantane” (also known as octahydro-2,5-methanopentalene) has a definition known to those skilled in the art, and its structural formula is e.g., as follows:

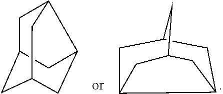

As used herein, “noradamantanyl” refers to a monovalent group of noradamantane, that is, the group remaining after any hydrogen in noradamantane is removed. Representative examples of “noradamantanyl” include, but are not limited to, 1-noradamantanyl, 2-noradamantanyl, 3-noradamantanyl, 4-noradamantanyl, 5-noradamantanyl, 6-noradamantanyl, 7-noradamantanyl, 8-noradamantanyl or 9-noradamantanyl.

As used herein, “adamantanamine” has the definitions known to those skilled in the art, namely referring to an adamantane having an amino substituent, wherein the amino substituent can replace a hydrogen on a carbon at any position in the adamantane. An example of “adamantanamine” can be adamantan-1-amine (corresponding English chemical name is adamantan-1-amine or Tricyclo[3.3.1.1^{3,7}]decan-1-amine; CAS No.: 768-94-5), with the following structural Formula:

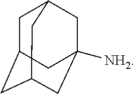 [SMILES: NC12CC3CC(CC(C3)C1)C2]

Salts or pharmaceutically acceptable salts, enantiomers, stereoisomers, solvates, polymorphs of the compounds of Formula (I) of the present disclosure are also encompassed within the scope of the present invention.

In all embodiments of the present disclosure, the salts or pharmaceutically acceptable salts of the compounds of Formula (I) refer to non-toxic inorganic or organic acid and/or base addition salts. Examples include: sulfate, hydrochloride, citrate, maleate, sulfonate, citrate, lactate, tartrate, fumarate, phosphate, dihydrogenphosphate, pyrophosphate, metaphosphate, oxalate, malonate, benzoate, mandelate, succinate, glycolate, or p-toluenesulfonate, etc.

“Pharmaceutically acceptable carrier” refers to a pharmaceutically acceptable material, such as a filler, stabilizer, dispersant, suspending agent, diluent, excipient, thickener, solvent, or encapsulating material, with which the useful compounds according to the present disclosure are carried or transported into or administered to a patient so that they can perform their intended function. Generally, such constructs are carried or transported from one organ or part of the body to another organ or part of the body. The carrier is compatible with the other ingredients of the formulation, including the compounds useful in the present disclosure, and is not harmful to the patient, and the carrier must be “acceptable”. Some examples of materials that can be used as pharmaceutically acceptable carriers include, but are not limited to, sugars such as lactose, glucose, and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository wax; oils such as peanut oil, cottonseed oil, safflower oil, sesame oil, olive oil, corn oil and soybean oil; glycols such as propylene glycol; polyols such as glycerol, sorbitol, mannitol, and polyethylene glycol; esters such as ethyl oleate and ethyl laurate; agar; buffers such as magnesium hydroxide and aluminum hydroxide; surfactant phosphate buffer solution; and other common non-toxic compatible substances used in pharmaceutical formulations.

As used herein, the term “room temperature” refers to the ambient temperature, such as 20-30° C.

As used herein, “stereoisomer” refers to a compound with the same chemical structural formula, but a different arrangement of atoms or groups in space. Stereoisomers include enantiomers, diastereomers, conformational isomers (rotational isomers), geometric isomers (cis/trans isomers), atropisomers, and so on.

As used herein, the term “solvate” refers to an association or complex formed by the interaction between one or more solvent molecules and compounds of the present invention. Examples of solvents include water, isopropanol, ethanol, methanol, DMSO, ethyl acetate, acetic acid and ethanolamine. The term “hydrate” means that a complex formed with water.

As used herein, the term “chiral” refers to a molecule that is nonsuperimposable on its mirror image; whereas “achiral” refers to a molecule that can be superimposed on its mirror image.

As used herein, the term “enantiomers” refers to two isomers of a compound that are nonsuperimposable mirror images.

As used herein, the term “diastereomers” refers to stereoisomers which have two or more chiral centers but which are non-mirror images. The diastereomers have different physical properties, such as melting point, boiling point, spectral properties and reactivity. The diastereomeric mixture can be separated by high-resolution analytical operations such as electrophoresis and chromatography, such as HPLC.

### Examples

In the following description, numerous specific details are set forth in order to provide a thorough understanding of the present disclosure. The present disclosure may be practiced without some or all of these specific details. In other cases, well-known process operations have not been described in detail in order not to unnecessarily obscure the present disclosure. Although the present disclosure will be described in conjunction with specific embodiments, it should be understood that this is not intended to limit the present disclosure to these embodiments.

The following abbreviations are used throughout the specification and examples:
Bipy bipyridine
Boc t-Butyloxy carbonyl
BnCl benzyl chloride
(COCl)₂ oxalyl chloride
Con. concentration
DCM dichloromethane
DMF N,N-dimethylformamide
DMSO dimethyl sulfoxide
DIPEA N, N-diisopropylethylamine
(COCl)₂ oxalyl chloride
EDCI 1-(3-dimethylaminopropyl)-3-ethylcarbodiimide
ESI electrospray ionization
equiv equivalent
EtOH ethanol
HATU 2-(7-Azabenzotriazol-1-yl)-N,N,N′,N′-tetramethyluronium hexafluorophosphate
HOAT N-Hydroxy-7-azabenzotriazole
HPLC high performance liquid chromatography
HRMS high resolution mass spectrometry
LC-MS liquid chromatography-mass spectrometry
LRMS low resolution mass spectrometry
LC liquid chromatography
Me methyl
MeCN acetonitrile
MeOH Methanol
MS mass spectrometry
MsCl methanesulfonyl chloride
MsO— methylsulfonyloxy
NMM N-methylmorpholine
NMP N-methylpyrrolidone
¹H NMR Proton nuclear magnetic resonance
—OTs p-toluenesulfonyloxy
PEG Polyethylene glycol chain
rt room temperature
tBu tert-butyl
t-BuONO tert-butyl nitrite
TEA triethylamine
TFA trifluoroacetate
TLC thin layer chromatography
TMS tetramethylsilane

In the present disclosure, the ¹H NMR spectrum was recorded on a Bruker-500 MHz nuclear magnetic resonance instrument, by using, as a solvent, CD₃OD (δ=3.31 ppm) containing 0.1% TMS (as an internal standard); or using, as a solvent, CDCl₃ (δ=7.26 ppm) containing 0.1% TMS (as an internal standard); or using, as a solvent, DMSO-d₆ (δ=2.50 ppm) containing 0.03% TMS (as an internal standard). LRMS spectrum was recorded on an AB Triple 4600 mass spectrometer, HPLC preparation was measured on a SHIMADZU LC-20AP type instrument, and HPLC purity was measured on a SHIMADZU LC-30AP or Waters 1525 type instrument. Unless otherwise specified, all reactions were performed in the air atmosphere. The reactions were followed by TLC or LC-MS.

Solvents and reagents are processed as follows:
the solvents used in the reactions such as DCM, DMF, anhydrous EtOH, and anhydrous MeOH were commercially available, e.g., from Chinese Sinopharm Group;
HPLC preparation was performed by using preparative grade CH₃CN and deionized water;
unless otherwise specified, Daporinad, various carbon chain link units with different lengths (i.e., compounds used to form the groups represented by LIN), as well as other reaction substrates, reagents and drugs were commercially available.
Unless otherwise specified, the materials and reagents used in the following examples are commercially available and used directly, or can be synthesized by using methods known in the art.

### General Synthesis Methods

Compounds and/or pharmaceutically acceptable salts thereof of the present disclosure can be synthesized using commercially available raw materials by synthetic techniques known in the art. The synthetic schemes described below illustrate the preparation of most compounds. The starting materials or reagents used in each scheme can be purchased from commercial sources or prepared by methods known to those skilled in the art. One skilled in the art can prepare the salt, racemate, enantiomer, phosphate, sulfate, hydrochloride and prodrug forms of the compounds of formula (I) of the present disclosure according to routine techniques in the art.

### General Synthesis Method for Daporinad Derivatives:

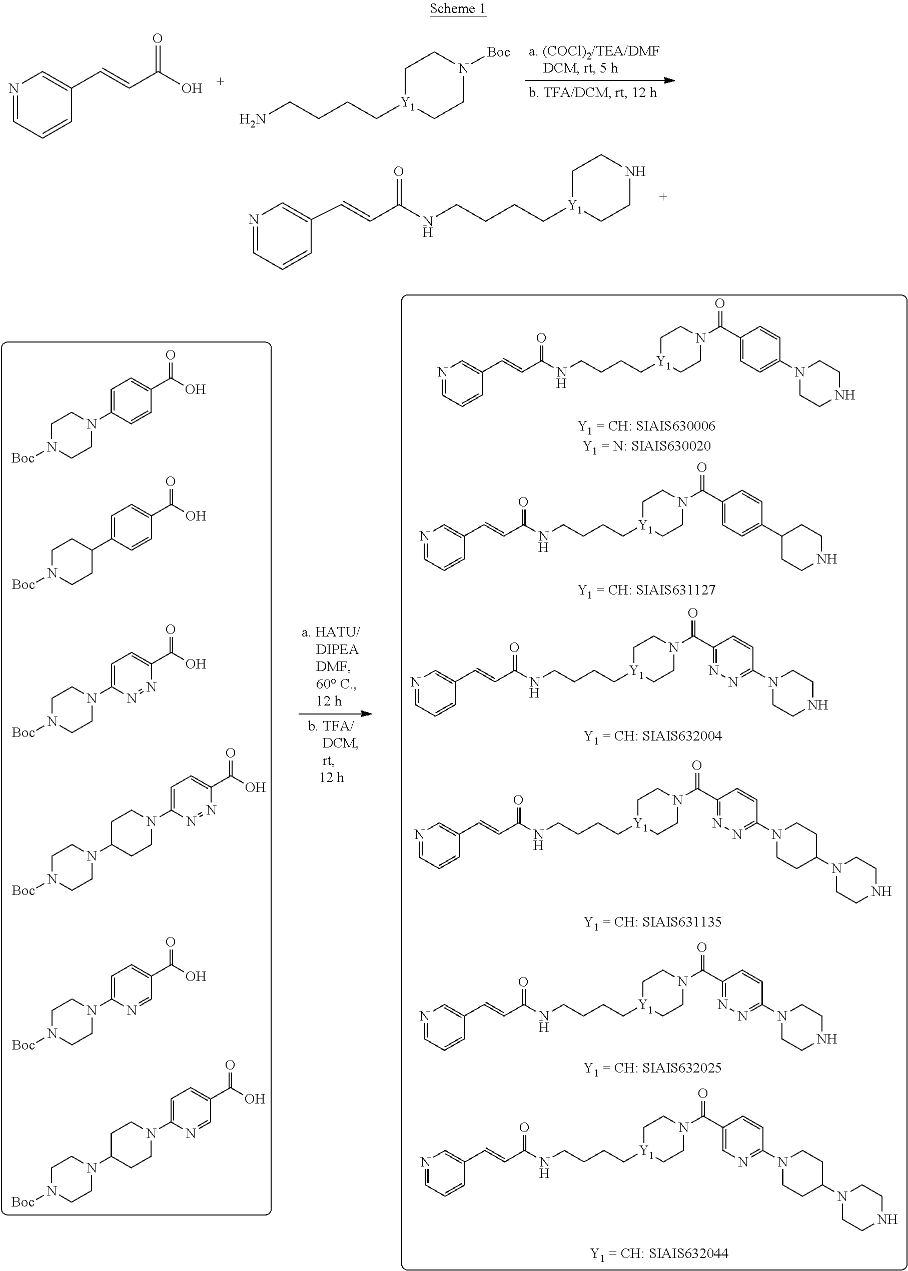

### General Synthesis Methods for Intermediates LM

General Synthesis Method for Intermediates LM (pomalidomide-N-Linker-COOH):

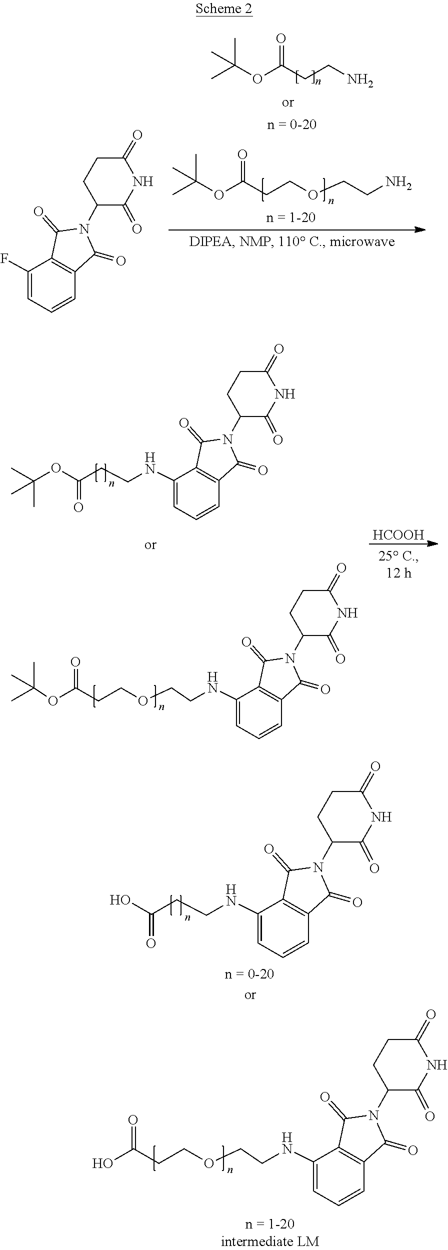

Step 1: a 30 mL microwave reaction tube was charged with 2-(2,6-dioxopiperidin-3-yl)-4-fluoroisoindoline-1,3-dione (1 equiv; CAS No.: 835616-60-9), the corresponding raw material amine (1.2 equiv), and N,N-diisopropylethylamine (5 equiv), followed by addition of NMP (8 mL). The resulting reaction mixture was stirred at room temperature for 10 minutes. Then argon gas was slowly blown into the microwave reaction tube. The microwave reaction tube was placed into a microwave reactor, and heated to 110° C. The reaction mixture was stirred for 2 h. The reaction solution was cooled to room temperature, and poured into 90% brine. The resulting mixture was extracted with ethyl acetate (4×50 mL). Organic phases were combined, washed with water (2×30 mL) and saturated brine (50 mL), and dried over anhydrous Na₂SO₄, and evaporated under reduced pressure to remove the solvent. The obtained crude product was purified by column chromatography (eluent (v/v): petroleum ether/ethyl acetate=1:1) to give Boc protected intermediate.

Step 2: a 50 mL single-necked flask was charged with the Boc protected intermediate from step 1 and 20 mL of 88% formic acid. The reaction mixture was stirred at room temperature for 12 h, and evaporated under reduced pressure to remove the reaction solvent. The resulting residue was added with water and lyophilized to obtain the corresponding final target compound.

General Synthesis Method for Intermediates LM (lenalidomide-N-Linker-COOH):

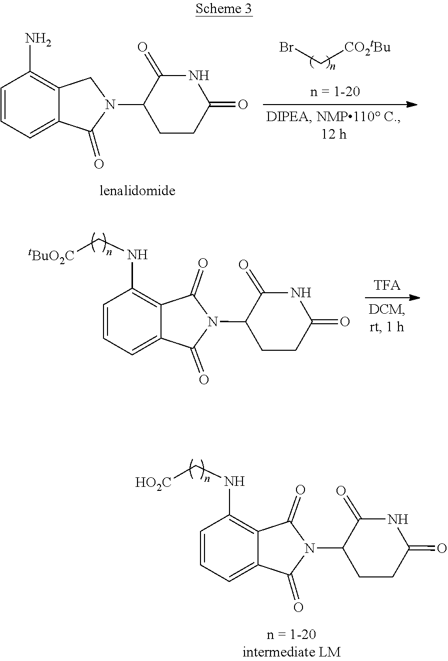

A single-necked flask was charged with lenalidomide (1 equiv.), NMP (8 mL), corresponding raw material brominated tert butyl ester (1.2 equiv.) and N,N-diisopropylethylamine (3 equiv.). The reaction solution was reacted at 110° C. for 12 h, cooled to room temperature, and subjected to a C18 reverse phase column (eluent (v/v): acetonitrile/(water+0.1% TFA)=10%-100%) for separation and purification, to give the corresponding intermediate tert butyl ester.

A single-necked flask was charged with the intermediate tert butyl ester from step 1, DCM (6 mL) and TFA (2 mL). The reaction mixture was stirred at room temperature for 1 h, and evaporated under reduced pressure to remove the reaction solvent. The resulting residue was added with water and lyophilized to obtain the corresponding final target compound.

General Synthesis Method for Intermediates LM (pomalidomide-N-alkylene-I):

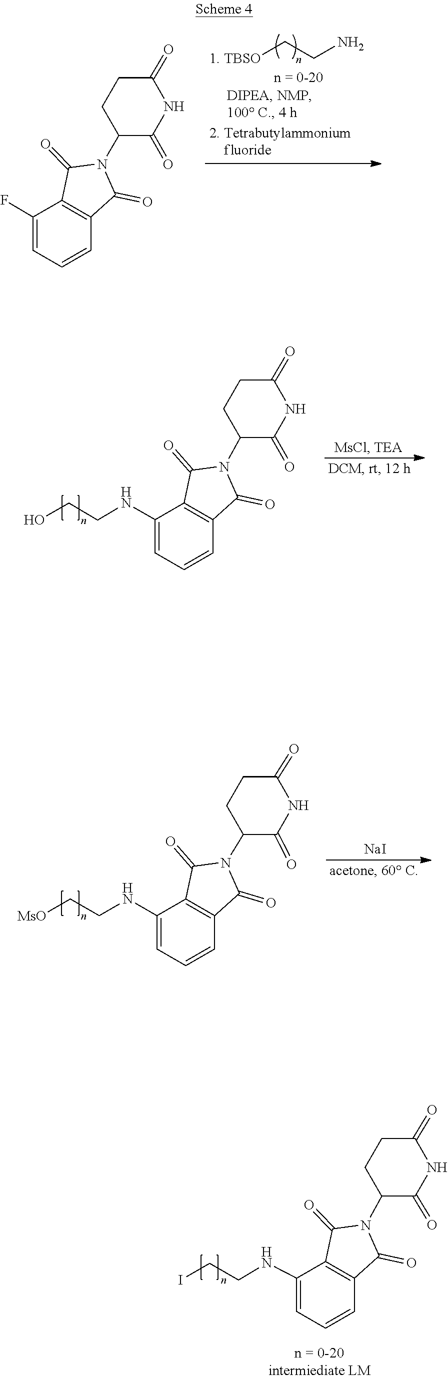

Step 1:

To a solution of 2-(2,6-dioxopiperidin-3-yl)-4-fluoroisoindoline-1,3-dione (1 equiv.) in NMP (25 mL) were sequentially added corresponding tert-butyldimethylsilyloxy-protected primary amine (1.0 equiv) and N,N-diisopropylethylamine (1.5 equiv.). The reaction mixture was reacted at 100° C. for 4 h. After the reaction was complete, the reaction solution was cooled to room temperature, and poured into saturated brine. The resulting mixture was extracted with ethyl acetate (4×50 mL). Organic phases were combined, washed with water (2×30 mL) and saturated brine (50 mL), and dried over anhydrous Na₂SO₄, and evaporated under reduced pressure to remove the solvent. The obtained crude product was purified by column chromatography (eluent (v/v): petroleum ether/ethyl acetate=1:1) to give the intermediate. To a solution of the intermediate in THF (50 mL) was added tetrabutylammonium fluoride (1 equiv.). The reaction mixture was stirred at room temperature for 2 h. After the reaction was complete, to the reaction solution was added saturated brine (200 mL). The resulting mixture was extracted with ethyl acetate (4×50 mL). Organic phases were combined, washed with water (2×30 mL) and saturated brine (50 mL), and dried over anhydrous Na₂SO₄, and evaporated under reduced pressure to remove the solvent. The obtained crude product was used directly in the next step.

Step 2:

To a solution of the crude product from step 1 in 40 mL of mixed solvent (DCM/pyridine=3/1) were sequentially added triethylamine (3.0 equiv) and methanesulfonyl chloride (1.5 equiv) in ice water bath. The reaction mixture was reacted at room temperature for 12 h. After the reaction was complete, the reaction solution was washed with saturated brine, and evaporated under reduced pressure to remove the solvent. The resulting residue was purified by column chromatography to give corresponding sulfonate intermediate.

Step 3: To a solution of the corresponding sulfonate intermediate from step 2 in acetone was added NaI. The reaction mixture was heated to 60° C. and reacted overnight to give the target compound.

General Synthesis Method for Intermediates LM (lenalidomide/pomalidomide-alkynyl-linker-OMs):

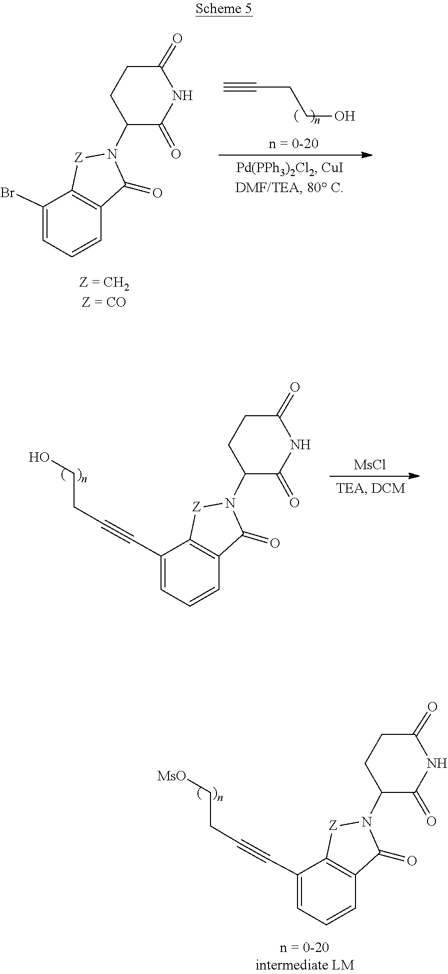

Step 1:

a solution of 3-(4-bromo-1-oxoisoindolin-2-yl)piperidine-2,6-dione or 4-bromo-2-(2,6-dioxopiperidin-3-yl)isoindoline-1,3-dione (1 equiv.) in DMF (5 mL) was bubbled with argon gas for 5 min, followed by sequentially addition of corresponding alkynol substrate (2 equiv.), Pd(PPh₃)₂Cl₂ (0.1 equiv.) and CuI (0.2 equiv.). The reaction mixture was stirred for 5 min, and then triethylamine (2.5 mL) was added thereto. The resulting mixture was heated to 80° C., and reacted overnight. The mixture was cooled to room temperature, quenched with 50 mL of water, and extracted with ethyl acetate (3×50 mL). Organic phases were combined, washed with water (2×30 mL) and saturated brine (50 mL), dried over anhydrous Na₂SO₄, and evaporated under reduced pressure to remove the solvent. The obtained crude product was purified by column chromatography (eluent (v/v): DCM/MeOH=5/1) to give the corresponding alcohol intermediate.

Step 2:

To a solution of the corresponding alcohol intermediate from step 1 in DCM (15 mL) were sequentially added triethylamine (3 equiv.) and methanesulfonyl chloride (1.5 equiv.). The reaction system become clear and reacted overnight. The reaction solution was washed with saturated brine, and evaporated under reduced pressure to remove the solvent. The resulting residue was purified by column chromatography (eluent (v/v): DCM/MeOH=5/1) to give the corresponding target compound.

General Synthesis Method for Intermediates LM (lenalidomide/pomalidomide-alkylene-OMs):

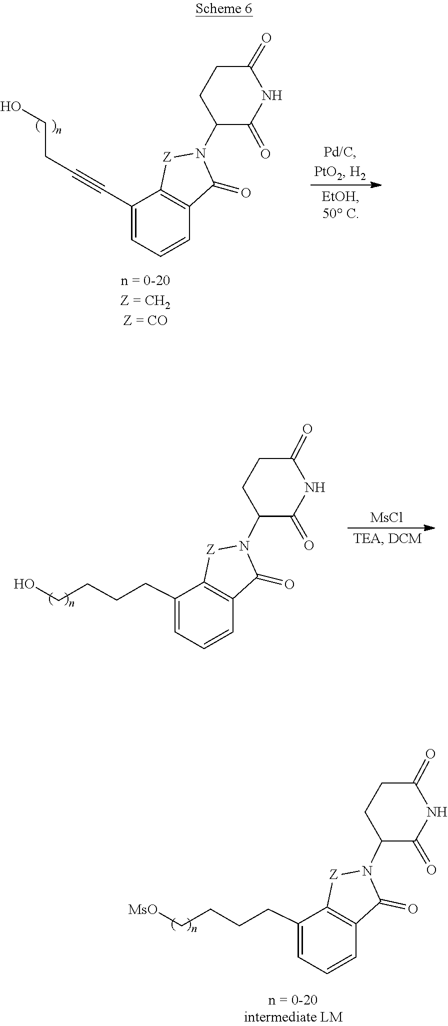

Step 1: to a solution of the corresponding alkynol substrate (1 equiv.) in ethanol (10 mL) were added 10% Pd/C (5 mg) and PtO₂ (5 mg) as catalysts under hydrogen atmosphere. The resulting mixture was reacted for 12 h at 50° C. under normal pressure under hydrogen atmosphere. The reaction mixture was filtered, and the filtrate was evaporated under reduced pressure to remove the solvent. The resulting crude product was directly used in the next step.

Step 2: to a solution of the reduction product from step 1 in DCM (15 mL) were sequentially added triethylamine (3 equiv.) and methanesulfonyl chloride (1.5 equiv.). The reaction system become clear and reacted overnight. The reaction solution was washed with saturated brine, and evaporated under reduced pressure to remove the solvent. The resulting residue was purified by column chromatography (eluent (v/v): DCM/MeOH=5/1) to give the corresponding target compound.

General Synthesis Method for Intermediates LM (pomalidomide-S-alkylene-COOH):

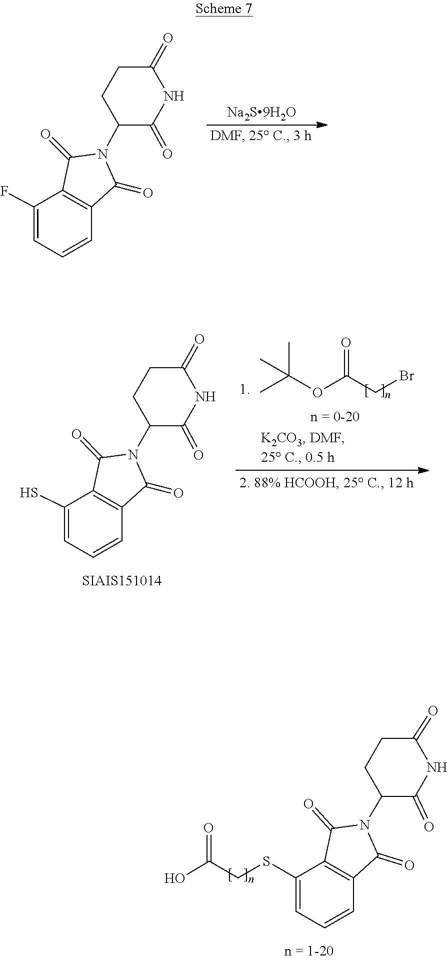

Step 1:

A 250 mL egg-shaped flask was charged with 2-(2,6-dioxopiperidin-3-yl)-4-fluoroisoindoline-1,3-dione (20 g, 72.4 mmol) and anhydrous N,N-dimethylformamide (150 mL), followed by addition of sodium sulfide nonahydrate (28 g, 108.6 mmol) in portions under stirring at room temperature, and the mixture was stirred at room temperature for 6 h. Then the reaction solution was slowly poured into 400 mL ice-water mixture. With stirring, the pH of the reaction solution was adjusted slowly to pH 2-3 with 6N aqueous hydrochloric acid solution. The color of the solution changed from blood red to light yellow, and a large amount of off-white solids were precipitated out. After stirring at room temperature for 0.5 h, the solids were filtrated, and the filter cake was washed 3 times with water; then the filter cake was slurried with 100 mL of anhydrous acetone, filtered, and washed 3 times with acetone, and dried under reduced pressure to afford intermediate 2-(2,6-dioxopiperidin-3-yl)-4-mercaptoisoindoline-1,3-dione (SIAIS151014).

Step 2:

A 100 mL egg-shaped flask was charged with the intermediate 2-(2,6-dioxopiperidin-3-yl)-4-mercaptoisoindoline-1,3-dione (SIAIS151014) (1 equiv) from step 1, anhydrous N,N-dimethylformamide (10 mL), and anhydrous potassium carbonate (2 equiv), followed by slow addition of the corresponding brominated substrate (1.2 equiv) under stirring at room temperature. After the completion of addition, the mixture was stirred at room temperature for 0.5 h. After the reaction was complete, 50 mL of water was poured into the reaction mixture, and the resulting mixture was extracted with ethyl acetate (2×50 mL). The organic phases were combined, washed with water (3×20 mL) and saturated brine (50 mL), dried over anhydrous sodium sulfate and concentrated under reduced pressure to remove the solvents. The crude product was purified by column chromatography (eluent (v/v): dichloromethane/ethyl acetate=20:1) to give the corresponding tert-butyl ester intermediate compound. The corresponding tert-butyl ester intermediate compound and 88% formic acid (10 mL) were sequentially added to a 25 mL egg-shaped flask, and stirred at room temperature for 12 h, and then concentrated under reduced pressure to remove the solvent. The resulting residue was added with water and lyophilized to afford the corresponding target compound.

General Synthesis Method for Intermediates LM (pomalidomide-S-PEGₙ-COOH):

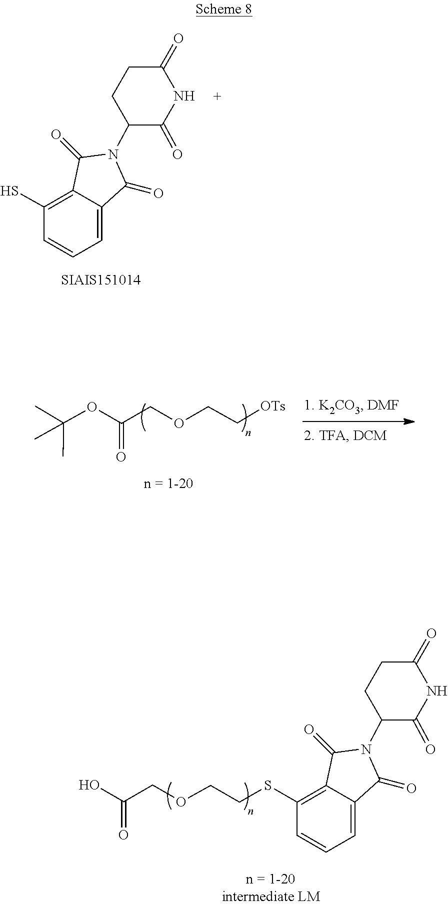

A 100 mL egg-shaped flask was charged with the 2-(2,6-dioxopiperidin-3-yl)-4-mercaptoisoindoline-1,3-dione (SIAIS151014) (1 equiv), anhydrous N,N-dimethylformamide (10 mL), and anhydrous potassium carbonate (2 equiv), followed by slow addition dropwise of the corresponding p-toluenesulfonate-protected PEG chain substrate (1.2 equiv), and the mixture was stirred at room temperature for 0.5 h. After the reaction was complete, 50 mL of water was poured into the reaction mixture, and the resulting mixture was extracted with ethyl acetate (2×50 mL). The organic phases were combined, washed with water (3×20 mL) and saturated brine (50 mL), dried over anhydrous sodium sulfate and concentrated under reduced pressure to remove the solvents. The crude product was purified by column chromatography (eluent (v/v): dichloromethane/ethyl acetate=20:1) to give the corresponding tert-butyl ester intermediate compound. A 25 mL egg-shaped flask was charged with the corresponding tert-butyl ester intermediate compound, followed by addition of DCM and TFA. The resulting mixture was stirred at room temperature for 1 h, and then concentrated under reduced pressure to remove the solvent. The resulting residue was added with water and lyophilized to afford the corresponding target compound.

General Synthesis Method for Intermediates LM (lenalidomide-S-alkylene-COOH):

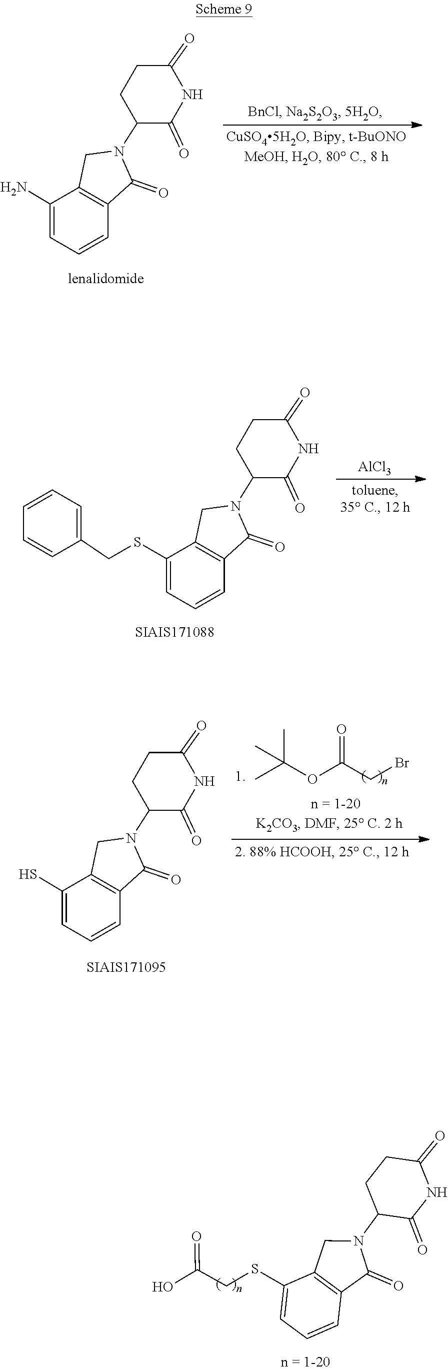

Step 1: a 500 mL egg-shaped flask containing methanol (120 mL) and water (120 mL) was charged with sodium thiosulfate pentahydrate (53.7 g, 216.3 mmol), benzyl chloride (27.4 g, 216.3 mmol), copper sulfate pentahydrate (77.4 mg, 0.31 mmol), and bipyridine (0.72 g, 4.6 mmol). The mixture was slowly warmed up to 80° C. and stirred for 2 h. After cooling to room temperature, to the reaction solution was added 3-(4-amino-1-oxoisoindolin-2-yl)piperidine-2,6-dione (lenalidomide) (8.0 g, 30.9 mmol), followed by slow addition dropwise of tert-butyl nitrite (4.78 g, 46.4 mmol). After the completion of addition, the reaction solution was warmed up to 80° C. again and stirred for 8 h. After the reaction was complete, the reaction solution was cooled to room temperature, followed by addition of water (200 mL), and extraction with ethyl acetate (2×200 mL). The organic phases were combined, washed with water (2×50 mL) and saturated brine (50 mL), dried over anhydrous sodium sulfate and concentrated under reduced pressure to remove the solvent. The crude product was purified by column chromatography (eluent (v/v): petroleum ether/ethyl acetate=1:2) to give the product (SIAIS171088).

Step 2: a 250 mL egg-shaped flask was charged with anhydrous aluminum trichloride (2.61 g, 19.6 mmol) and anhydrous toluene (70 mL), followed by slow addition of the compound SIAIS171088 (1.8 g, 4.9 mmol) with stirring. The mixture was stirred at 35° C. overnight. After the reaction was completed, 20% citric acid aqueous solution was slowly added to the reaction mixture with stirring, and a large amount of solids were precipitated out. After filtration, the filter cake was washed with water and ethyl acetate, respectively, and dried to give the product (SIAIS171095).

Step 3: a 100 mL egg-shaped flask was charged with the compound SIAIS171095 (1 equiv), anhydrous N,N-dimethylformamide (10 mL) and anhydrous potassium carbonate (2 equiv), followed by slowly addition dropwise of the corresponding brominated substrate (1.2 equiv). The mixture was stirred at room temperature for 0.5 h. After the reaction was complete, 50 mL of water was poured into the reaction mixture, and the resulting mixture was extracted with ethyl acetate (2×50 mL). The organic phases were combined, washed with water (3×20 mL) and saturated brine (50 mL), dried over anhydrous sodium sulfate and concentrated under reduced pressure to remove the solvent. The crude product was purified by a column chromatography (eluent (v/v): dichloromethane/ethyl acetate=20:1) to give the corresponding tert-butyl ester intermediate compound. The corresponding tert-butyl ester intermediate compound and 88% formic acid (10 mL) were sequentially added to a 25 mL egg-shaped flask, and stirred at room temperature for 12 h, and then concentrated under reduced pressure to remove the solvent. The resulting residue was treated by addition of water and lyophilized to afford the corresponding target compound.

General Synthesis Method for Intermediates LM (lenalidomide-S-PEGₙ-COOH):

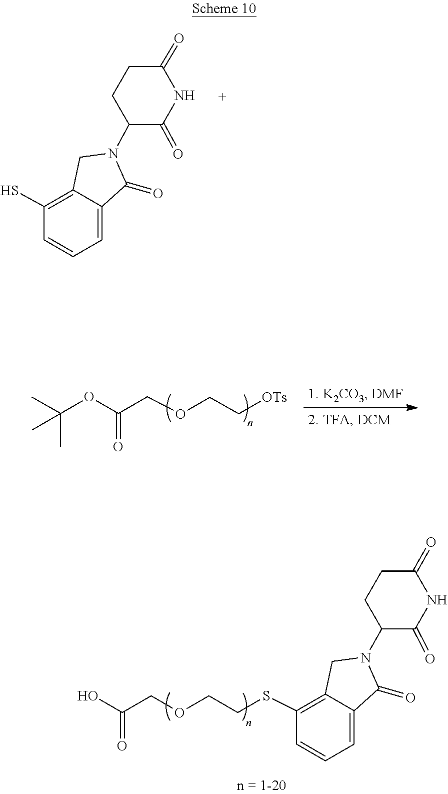

A 100 mL egg-shaped flask was charged with the intermediate SIAIS171095 (1 equiv), anhydrous N,N-dimethylformamide (10 mL), and anhydrous potassium carbonate (2 equiv), followed by slow addition dropwise of the corresponding p-toluenesulfonate-protected PEG chain substrate (1.2 equiv) with stirring at room temperature. After the completion of addition, the mixture was stirred at room temperature for 0.5 h. After the reaction was complete, 50 mL of water was poured into the reaction mixture, and the resulting mixture was extracted with ethyl acetate (2×50 mL). The organic phases were combined, washed with water (3×20 mL) and saturated brine (50 mL), dried over anhydrous sodium sulfate and concentrated under reduced pressure to remove the solvent. The crude product was purified by a column chromatography (eluent (v/v): dichloromethane/ethyl acetate=20:1) to give the corresponding tert-butyl ester intermediate compound. A 25 mL egg-shaped flask was charged with the corresponding tert-butyl ester intermediate compound, followed by addition of DCM (10 mL) and TFA (5 mL). The resulting mixture was stirred at room temperature for 12 h, and then concentrated under reduced pressure to remove the solvent. The resulting residue was added with water and lyophilized to afford the corresponding target compound.

General Synthesis Method for Intermediates LM (lenalidomide/pomalidomide-S-alkylene-Br):

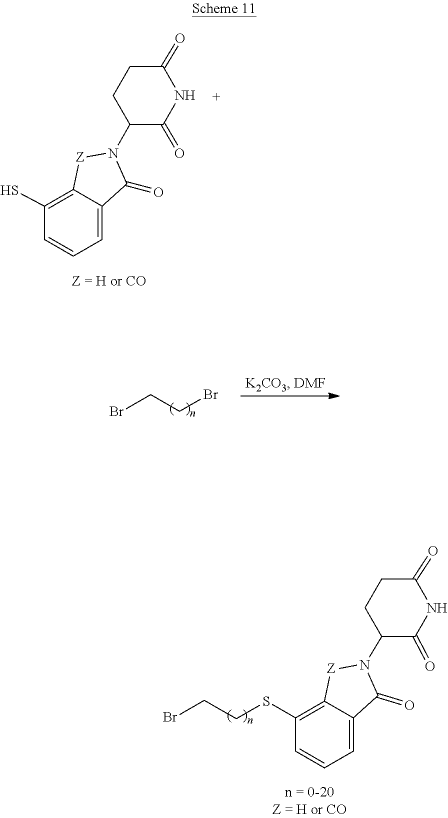

To a solution of 3-(4-mercapto-1-oxoisoindolin-2-yl)piperidine-2,6-dione or 2-(2,6-dioxopiperidin-3-yl)-4-mercaptoisoindoline-1,3-dione (1 equiv) in DMF (20 mL) was added K₂CO₃ (3 equiv.). The reaction mixture was stirred for 15 min, and the corresponding dibromide substrate (3 equiv) was added thereto. The reaction solution was reacted at room temperature for 12 h, and then was poured into 50 mL of water. The resulting mixture was extracted with dichloromethane twice. Organic phases were combined, washed with saturated brine, and evaporated under reduced pressure to remove the solvent. The obtained residue was purified by column chromatography (eluent (v/v): DCM to DCM/MeOH (10/1)) to give the corresponding target compound.

General Synthesis Method for Intermediates LM (lenalidomide-O-alkylene-Br):

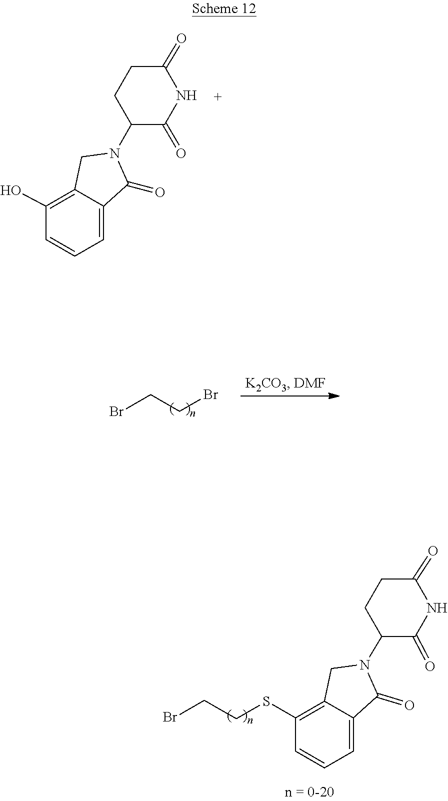

To a solution of 3-(4-hydroxy-1-oxoisoindolin-2-yl)piperidine-2,6-dione (1 equiv) in DMF (20 mL) was added K₂CO₃ (3 equiv.). The reaction mixture was stirred for 15 min, and the corresponding dibromide substrate (3 equiv) was added thereto. The reaction solution was heated to 80° C. and reacted for 12 h, and then poured into 50 mL of water. The resulting mixture was extracted with dichloromethane twice. Organic phases were combined, washed with saturated brine, and evaporated under reduced pressure to remove the solvent. The obtained residue was purified by column chromatography (eluent (v/v): DCM to DCM/MeOH (10/1)) to give the corresponding target compound.

General Synthesis Method for Intermediates LM (pomalidomide-O-alkylene-COOH):

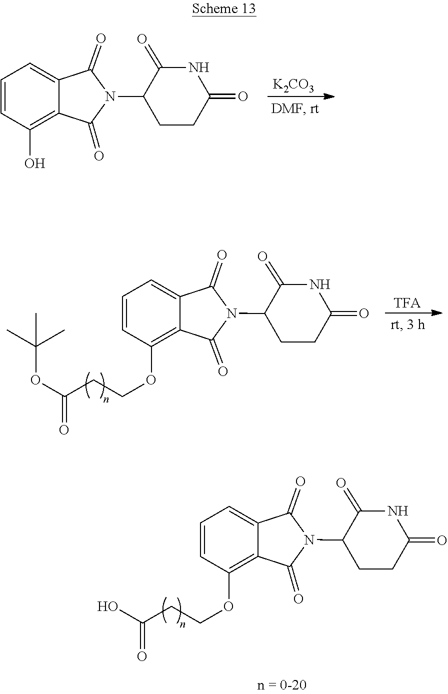

Step 1: to a solution of 2-(2,6-dioxopiperidin-3-yl)-4-hydroxyisoindoline-1,3-dione (1 equiv) in DMF (20 mL) were added K₂CO₃ (3 equiv.) and the corresponding tert-butyl-protected brominated alkyl chain acid substrate (1.2 equiv). The reaction solution was reacted at room temperature for 2 h, and then poured into 50 mL of water. The resulting mixture was extracted with dichloromethane twice. Organic phases were combined, washed with saturated brine, and evaporated under reduced pressure to remove the solvent. The obtained residue was purified by column chromatography (eluent (v/v): DCM to DCM/MeOH (10/1)) to give the corresponding tert-butyl ester intermediate compound.

Step 2: a 25 mL egg-shaped flask was charged with the corresponding tert-butyl ester intermediate compound, followed by addition of DCM (10 mL) and TFA (5 mL). The resulting mixture was stirred at room temperature for 12 h, and then concentrated under reduced pressure to remove the solvent. The resulting residue was added with water and lyophilized to afford the corresponding target compound.

### General Synthesis Method for Intermediates LM (VHL-CO-Linker-COOH):

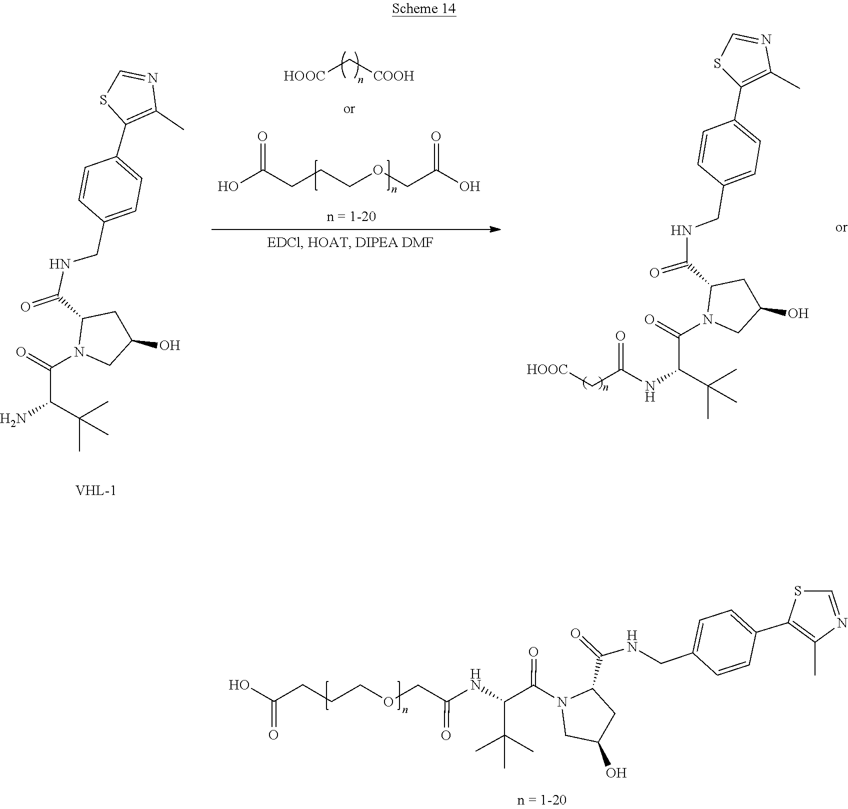

A 250 mL three-necked flask was charged with the corresponding raw material diacid (2.5 equiv), anhydrous DMF (10 mL) and anhydrous dichloromethane (150 mL), followed by addition of DIPEA (10.0 mmol, 5 equiv), VHL-1 (2 mmol, 1 equiv), HOAT (2.4 mmol, 1.2 equiv), and EDCI (2.4 mmol, 1.2 equiv) with stirring in ice water bath. After the completion of addition, the reaction mixture was stirred in ice water bath for 5 h, and then warmed up to room temperature and stirred overnight. After the reaction was complete, the reaction was quenched with 1 mL of deionized water, and evaporated under reduced pressure to remove the dichloromethane. The resulting residue was subjected to a C18 reverse phase column (eluent (v/v): acetonitrile/(water+0.1% TFA)=10%-100%) for separation and purification. The collected fractions were concentrated under reduced pressure to remove acetonitrile, and the resulting residue was lyophilized to give the corresponding target compound.

### General Synthesis Method for the Compounds of the Present Invention:

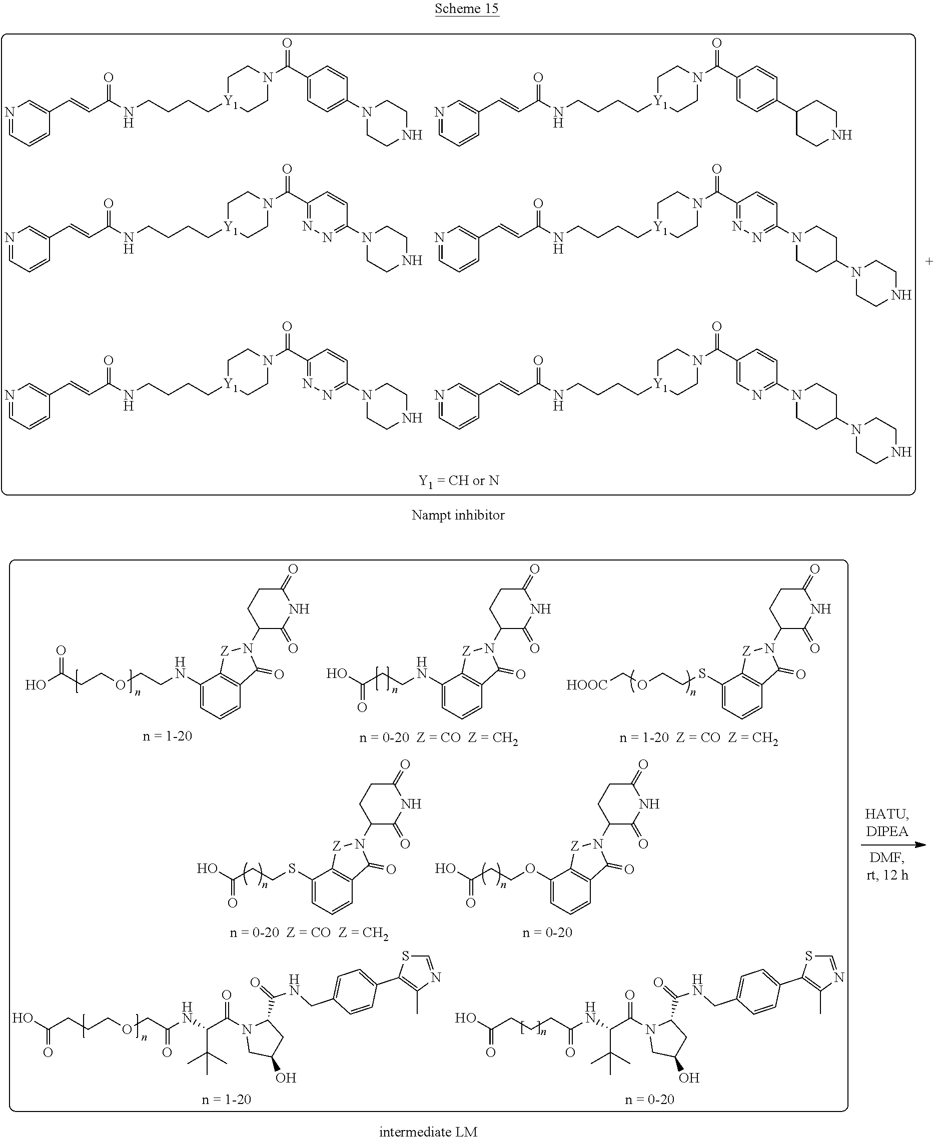

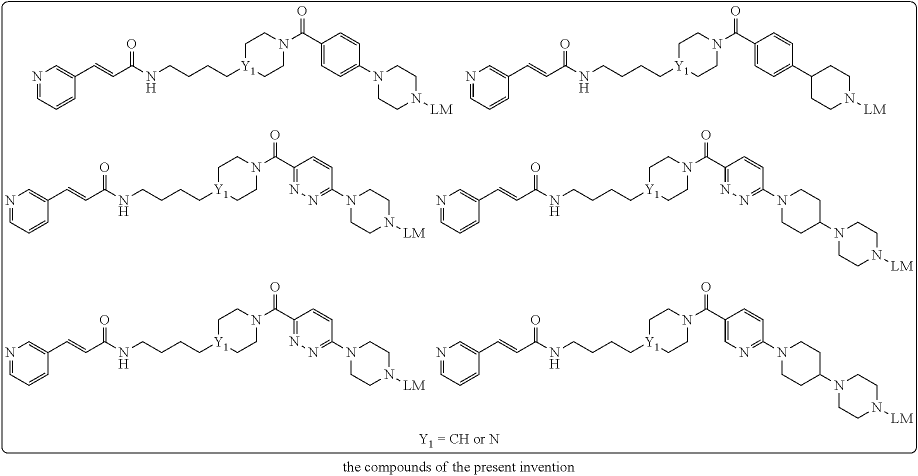

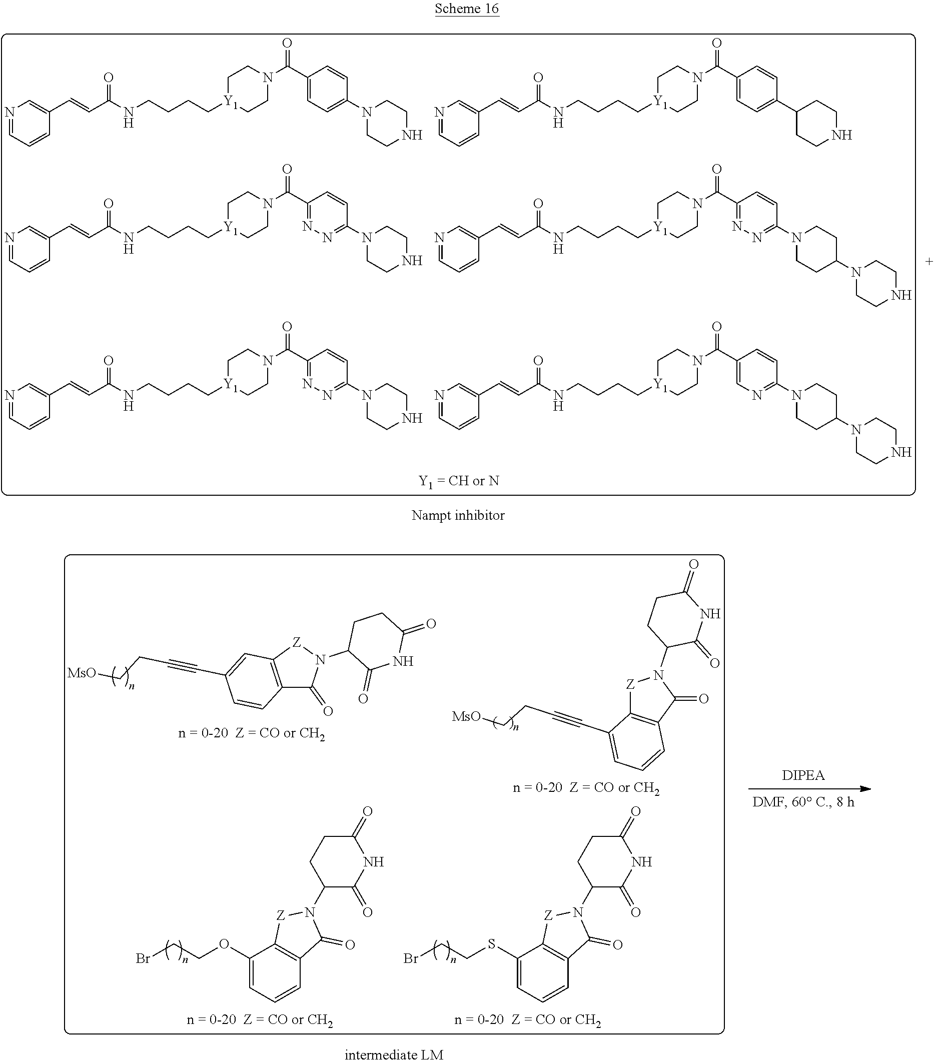

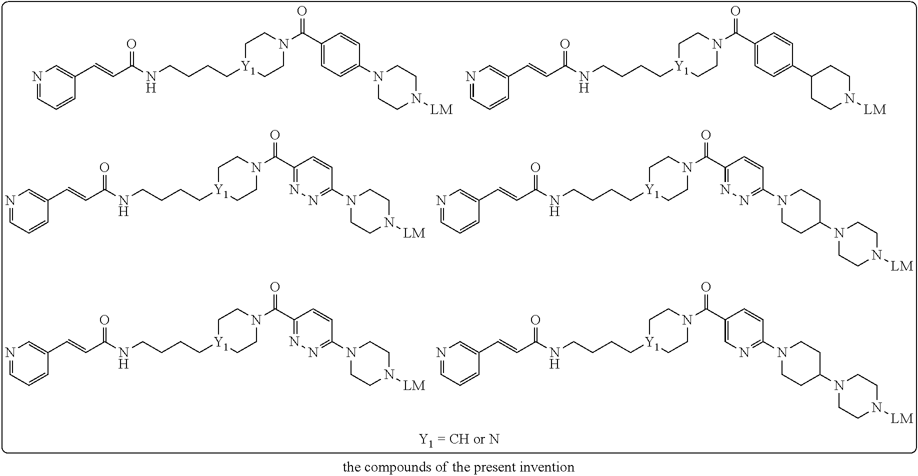

### General Synthesis Method for Intermediates:

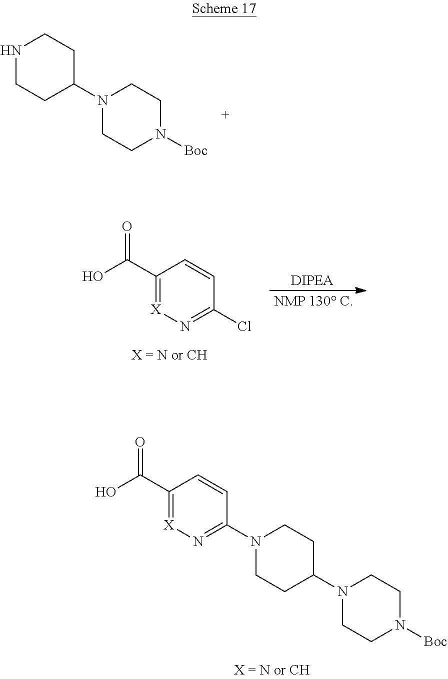

Depending upon the target compounds, the above schemes and their reaction substrates, reaction conditions (including reaction dosage, temperature, duration, etc.), work up, etc. can be appropriately modified and adjusted by techniques and methods well known to those skilled in the art to obtain the desired target compounds. The obtained target compounds can be further modified by the substituents and the like to obtain subsequent target compounds through methods well known to those skilled in the art.

### INTERMEDIATE PREPARATION EXAMPLES

### Intermediate Preparation Example 1: Preparation of Daporinad Derivative 1 (SIAIS630006)

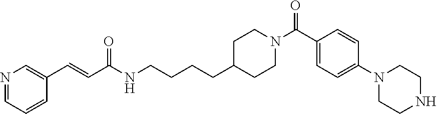 [SMILES: O=C(/C=C/c1cccnc1)NCCCCC1CCN(C(=O)c2ccc(N3CCNCC3)cc2)CC1]

Referring to the method of Scheme 1, Daporinad derivative 1 (SIAIS630006) was prepared.

### Step 1: Preparation of (E)-N-(4-(piperidin-4-yl)butyl)-3-(pyridin-3-yl)acrylamide (SIAIS524135)

A 50 ml round-bottomed flask was sequentially charged with (E)-3-(pyridin-3-yl)acrylic acid (1 g, 1.5 equiv) and DCM (10 mL) at room temperature, followed by addition dropwise of oxalyl chloride (0.7 mL, 1.8 equiv) in ice bath. After the addition was complete, 2 drops of DMF were added to the reaction mixture to initiate the reaction, and the reaction mixture was reacted for 3 hours at 60° C. A small amount of the reaction solution was taken, dissolved in methanol, and analyzed by LC-MS to detect the formation of the corresponding methyl ester. The reaction solution was then evaporated to remove DCM for further use. Separately, another 50 mL round-bottomed flask was charged with tert-butyl 4-(4-aminobutyl)piperidine-1-carboxylate (860 mg, 0.75 equiv), 10 mL of DCM, and 1 mL of TEA (1.5 equiv), followed by slow addition dropwise of the acyl chloride solution dissolved in DCM under an ice bath. After the addition was complete, the reaction mixture was reacted for 1 hour at room temperature. The reaction was monitored by LC-MS, and upon completion, water was added to quench the reaction. To the mixture was added Silica gel, and the resulting mixture was then subjected to a column chromatography using an eluent system of 5% CH₃OH/DCM to yield a yellow oily compound, which was directly used for the next reaction. To the solution of this yellow oily compound in 10 mL of DCM was added 2 mL of TFA, and the reaction mixture was reacted at room temperature for 2 hours. Once the reaction was complete as monitored by LC-MS, the reaction solution was rotary evaporated to remove the reaction solvent. The resulting residue was adjusted to a basic pH with a saturated solution of sodium bicarbonate, and then extracted three times with dichloromethane/methanol (10:1). The organic phases were combined, washed twice with a saturated NaCl solution, dried over anhydrous sodium sulfate, and filtered. The filtrate was evaporated to dryness, and the resulting residue was subjected to C18 reverse-phase column chromatography using CH₃CN and water as the eluent for separation. The collected fractions were evaporated to remove CH₃CN, and the resulting residue was lyophilized to afford SIAIS524135 as white solid (823 mg, total yield for two steps: 64%). ¹H NMR (500 MHz, CD₃OD) δ 9.12 (s, 1H), 8.85-8.80 (m, 2H), 8.12-7.98 (m, 1H), 7.63 (d, J=15.8 Hz, 1H), 7.02 (d, J=15.9 Hz, 1H), 3.44-3.27 (m, 5H), 3.05-2.95 (m, 2H), 1.95 (dd, J=13.6, 3.4 Hz, 2H), 1.60 (p, J=7.1 Hz, 3H), 1.45-1.27 (m, 7H). HRMS (ESI) calcd for C₁₇H₂₆N₃O⁺ [M+H]⁺, 288.2070; found, 288.2074.

### Step 2: Preparation of (E)-N-(4-(1-(4-(piperazin-1-yl)benzoyl)piperidin-4-yl)butyl)-3-(pyridin-3-yl)acrylamide (SIAIS630006)

A 50 ml round-bottomed flask was sequentially charged with SIAIS524135 (860 mg, 1 equiv), 4-(4-(tert-butoxycarbonyl)piperazin-1-yl)benzoic acid (881 mg, 1 equiv), HATU (1.36. g, 1.2 equiv), 5 mL of DMF, and 1.6 mL of DIPEA (3 equiv) at room temperature. The reaction mixture was reacted at 60° C. overnight. Once the reaction was complete as monitored by LC-MS, the reaction mixture was filtered, and the filtrate was subjected to a C18 reverse-phase column using CH₃CN and water as the eluent for separation. The collected fractions were evaporated to remove CH₃CN, and the resulting residue was lyophilized to yield a yellow solid compound, which was directly used for the next reaction. To the solution of this yellow solid compound in 10 mL of DCM was added 2 mL of TFA, and the reaction mixture was reacted at room temperature for 2 hours. Once the reaction was complete as monitored by LC-MS, the reaction solution was rotary evaporated to remove the reaction solvent. The resulting residue was adjusted to a basic pH with a saturated solution of sodium bicarbonate, and then the organic phases were combined, extracted three times with dichloromethane/methanol (10:1), washed twice with a saturated NaCl solution, dried over anhydrous sodium sulfate, and filtered. The filtrate was evaporated to dryness, and the resulting residue was subjected to C18 reverse-phase column chromatography using CH₃CN and water as the eluent for separation. The collected fractions were evaporated to remove CH₃CN, and the resulting residue was lyophilized to afford SIAIS630006 as yellow solid (1.1 g, total yield for two steps: 64%). ¹H NMR (500 MHz, CD₃OD) δ 9.13 (s, 1H), 8.87 (dd, J=18.8, 6.9 Hz, 2H), 8.15 (dd, J=8.2, 5.7 Hz, 1H), 7.64 (d, J=15.8 Hz, 1H), 7.50-7.42 (m, 2H), 7.26-7.16 (m, 2H), 7.05 (d, J=15.8 Hz, 1H), 4.98 (br, 4H), 3.63 (s, 4H), 3.43 (s, 4H), 3.34 (t, J=7.0 Hz, 2H), 1.94-1.75 (m, 2H), 1.70-1.56 (m, 3H), 1.49-1.40 (m, 2H), 1.40-1.32 (m, 2H), 1.31-1.19 (m, 2H). HRMS (ESI) calcd for C₂₈H₃₈N₅O₂⁺ [M+H]⁺, 476.3020; found, 476.3024.

### Intermediate Preparation Example 2: Preparation of Daporinad Derivative 2 (SIAIS630020)

Referring to Scheme 1 and using a method similar to the preparation of the Daporinad derivative 1 in Intermediate Preparation Example 1, the Daporinad derivative 2 was prepared, and the synthesis data and structural characterization data thereof are as follows:

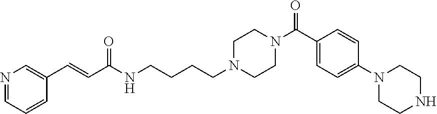 [SMILES: O=C(/C=C/c1cccnc1)NCCCCN1CCN(C(=O)c2ccc(N3CCNCC3)cc2)CC1]

(E)-N-(4-(4-(4-(piperazin-1-yl)benzoyl)piperazin-1-yl)butyl)-3-(pyridin-3-yl)acrylamide (SIAIS630020). (white solid, 258 mg, total yield for two steps: 63%). ¹H NMR (500 MHz, CD₃OD) δ 9.07 (s, 1H), 8.82-8.77 (m, 2H), 8.08 (dd, J=8.3, 5.6 Hz, 1H), 7.94 (d, J=9.0 Hz, 2H), 7.66 (d, J=15.9 Hz, 1H), 7.06 (d, J=9.0 Hz, 2H), 6.99 (d, J=15.9 Hz, 1H), 3.65 (br, 3H), 3.61-3.56 (m, 4H), 3.56-3.51 (m, 1H), 3.44-3.36 (m, 7H), 3.42-3.36 (m, 5H), 1.91-1.86 (m, 2H), 1.73-1.68 (m, 2H). HRMS (ESI) calcd for C₂₇H₃₇N₆O₂⁺ [M+H]⁺: 477.2973, found, 477.2975.

### Intermediate Preparation Example 3: Preparation of Daporinad Derivative 3 (SIAIS631127)

Referring to Scheme 1 and using a method similar to the preparation of the Daporinad derivative 1 in Intermediate Preparation Example 1, the Daporinad derivative 3 was prepared, and the synthesis data and structural characterization data thereof are as follows:

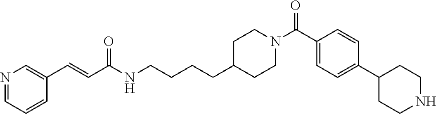 [SMILES: O=C(/C=C/c1cccnc1)NCCCCC1CCN(C(=O)c2ccc(C3CCNCC3)cc2)CC1]

(E)-N-(4-(1-(4-(piperidin-4-yl)benzoyl)piperidin-4-yl)butyl)-3-(pyridin-3-yl)acrylamide (SIAIS631127) (yellow oily liquid, 359 mg, yield 73%). ¹H NMR (500 MHz, CD₃OD) δ 9.12 (s, 1H), 8.92-8.83 (m, 2H), 8.15 (dd, J=8.2, 5.6 Hz, 1H), 7.64 (d, J=15.8 Hz, 1H), 7.44-7.30 (m, 4H), 7.03 (d, J=15.9 Hz, 1H), 4.60 (d, J=12.9 Hz, 1H), 3.71 (d, J=13.3 Hz, 1H), 3.52 (d, J=13.1 Hz, 2H), 3.34 (t, J=7.1 Hz, 2H), 3.20-3.10 (m, 3H), 3.02-2.94 (m, 1H), 2.85 (t, J=12.8 Hz, 1H), 2.11-2.04 (m, 2H), 2.03-1.91 (m, 2H), 1.88-1.82 (m, 1H), 1.73-1.67 (m, 1H), 1.64-1.56 (m, 3H), 1.48-1.40 (m, 2H), 1.38-1.32 (m, 2H), 1.25-1.06 (m, 2H). HRMS (ESI) calcd for C₂₉H₃₉N₄O₂⁺ [M+H]⁺: 475.3068, found, 475.3072.

### Intermediate Preparation Example 4: Preparation of Daporinad Derivative 4 (SIAIS632004)

Referring to Scheme 1 and using a method similar to the preparation of the Daporinad derivative 1 in Intermediate Preparation Example 1, the Daporinad derivative 4 was prepared, and the synthesis data and structural characterization data thereof are as follows:

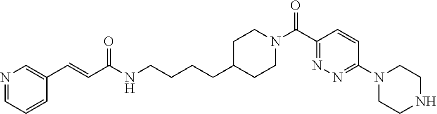 [SMILES: O=C(/C=C/c1cccnc1)NCCCCC1CCN(C(=O)c2ccc(N3CCNCC3)nn2)CC1]

(E)-N-(4-(1-(6-(piperazin-1-yl)pyridazine-3-carbonyl)piperidin-4-yl)butyl)-3-(pyridin-3-yl)acrylamide (SIAIS632004) (yellow oily liquid, 359 mg, yield 73%). ¹H NMR (500 MHz, CD₃OD) δ 8.77 (s, 1H), 8.57 (d, J=5.0 Hz, 1H), 8.18 (d, J=8.0 Hz, 1H), 7.64 (d, J=9.5 Hz, 1H), 7.60-7.52 (m, 2H), 7.43 (d, J=9.5 Hz, 1H), 6.76 (d, J=15.8 Hz, 1H), 4.63 (d, J=12.9 Hz, 1H), 3.99 (t, J=5.3 Hz, 3H), 3.94 (d, J=14.3 Hz, 1H), 3.44 (p, J=1.6 Hz, 1H), 3.39-3.36 (m, 4H), 3.33 (br, 3H), 3.16 (p, J=1.7 Hz, 2H), 1.88 (d, J=13.8 Hz, 1H), 1.72 (d, J=13.5 Hz, 1H), 1.60-1.56 (m, 3H), 1.48-1.43 (m, 2H), 1.37-1.33 (m, 2H), 1.25-1.22 (m, 2H). HRMS (ESI) calcd for C₂₆H₃₆N₇O₂⁺ [M+H]⁺: 478.2925, found, 478.2928.

### Intermediate Preparation Example 5: Preparation of Daporinad Derivative 5 (SIAIS631135)

### Step 1: Preparation of 6-(4-(4-(tert-butoxycarbonyl)piperazin-1-yl)piperidin-1-yl)pyridazine-3-carboxylic Acid (SIAIS631145)

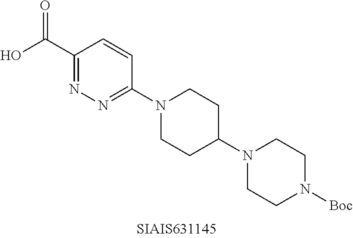 [SMILES: CC(C)(C)OC(=O)N1CCN(C2CCN(c3ccc(C(=O)O)nn3)CC2)CC1]

Referring to Scheme 17, 6-(4-(4-(tert-butoxycarbonyl)piperazin-1-yl)piperidin-1-yl)pyridazine-3-carboxylic acid (SIAIS631145) was prepared.

To a solution of 6-chloropyridazine-3-carboxylic acid (1 equiv.) in DMF (20 mL) were added DIPEA (5 equiv) and tert-butyl 4-(piperidin-4-yl)piperazine-1-carboxylate (2 equiv). The reaction mixture was reacted at 130° C. for 3 h, then was slowly poured into 50 mL of water. The resulting mixture was extracted twice with dichloromethane. The organic phases were combined, washed with saturated brine, and concentrated under reduced pressure to remove the solvents. The resulting residue was purified by column chromatography (eluent (v/v): DCM to DCM/MeOH (10/1)) to give the intermediate compound SIAIS631145, ESI [M+H]⁺: 392.

### Step 2: Preparation of (E)-N-(4-(1-(6-(4-(piperazin-1-yl)piperidin-1-yl)pyridazine-3-carbonyl)piperidin-4-yl)butyl)-3-(pyridin-3-yl)acrylamide (SIAIS631135)

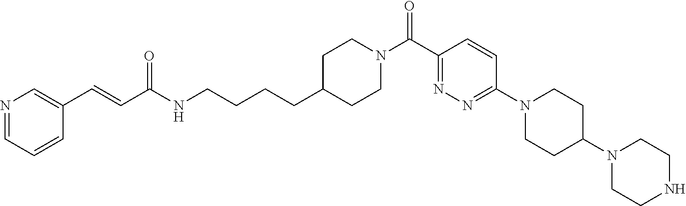 [SMILES: O=C(/C=C/c1cccnc1)NCCCCC1CCN(C(=O)c2ccc(N3CCC(N4CCNCC4)CC3)nn2)CC1]

Referring to Scheme 1 and using a method similar to the preparation of the Daporinad derivative 1 in Intermediate Preparation Example 1, the Daporinad derivative 5 ((E)-N-(4-(1-(6-(4-(piperazin-1-yl)piperidin-1-yl)pyridazine-3-carbonyl)piperidin-4-yl)butyl)-3-(pyridin-3-yl)acrylamide (SIAIS631135)) was prepared by using the intermediate SIAIS631145 from step 1. (yellow oily liquid, 359 mg, yield 73%). ¹H NMR (500 MHz, CD₃OD) δ 9.11 (s, 1H), 8.95-8.80 (m, 2H), 8.17-7.97 (m, 3H), 7.64 (d, J=15.8 Hz, 1H), 7.01 (dd, J=15.8, 5.5 Hz, 1H), 4.60-4.53 (m, 2H), 4.02 (d, J=13.3 Hz, 1H), 3.87 (s, 1H), 3.72 (s, 7H), 3.46 (t, J=12.5 Hz, 2H), 3.34 (t, J=7.1 Hz, 3H), 3.20 (t, J=12.2 Hz, 1H), 3.02-2.85 (m, 1H), 2.48 (d, J=12.1 Hz, 2H), 2.19-2.01 (m, 2H), 1.98-1.85 (m, 1H), 1.79 (d, J=13.0 Hz, 1H), 1.69-1.55 (m, 3H), 1.50-1.32 (m, 4H), 1.30-1.17 (m, 2H). HRMS (ESI) calcd for C₃₁H₄₅N₈O₂⁺ [M+H]⁺: 561.3660, found 561.3665.

### Intermediate Preparation Example 6: Preparation of Daporinad Derivative 6 (SIAIS632025)

Referring to Scheme 1 and using a method similar to the preparation of the Daporinad derivative 1 in Intermediate Preparation Example 1, the Daporinad derivative 6 was prepared, and the synthesis data and structural characterization data thereof are as follows:

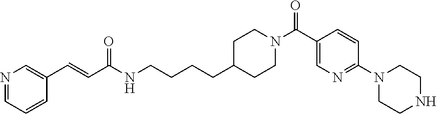 [SMILES: O=C(/C=C/c1cccnc1)NCCCCC1CCN(C(=O)c2ccc(N3CCNCC3)nc2)CC1]

(E)-N-(4-(1-(6-(piperazin-1-yl)nicotinoyl)piperidin-4-yl)butyl)-3-(pyridin-3-yl)acrylamide (SIAIS632025) (yellow oily liquid, 241 mg, yield 74%). ¹H NMR (500 MHz, CD₃OD) δ 9.14 (s, 1H), 8.92 (s, 1H), 8.73 (d, J=8.2 Hz, 1H), 8.25 (d, J=2.1 Hz, 1H), 8.09 (s, 1H), 7.73-7.59 (m, 2H), 7.00 (d, J=9.0 Hz, 1H), 6.92 (d, J=15.8 Hz, 1H), 3.97-3.90 (m, 4H), 3.84 (s, 2H), 3.35-3.32 (m, 8H), 1.80 (br, 2H), 1.63-1.56 (m, 3H), 1.46-1.40 (m, 2H), 1.39-1.28 (m, 2H), 1.19 (br, 2H). HRMS (ESI) calcd for C₂₇H₃₇N₆O₂⁺ [M+H]⁺: 477.2973, found, 477.2976.

### Intermediate Preparation Example 7: Preparation of Daporinad Derivative 7 (SIAIS632044)

### Step 1: Preparation of 6-(4-(4-(tert-butoxycarbonyl)piperazin-1-yl)piperidin-1-yl)nicotinic Acid (SIAIS631147)

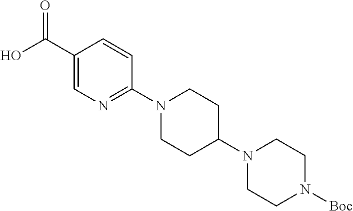 [SMILES: CC(C)(C)OC(=O)N1CCN(C2CCN(c3ccc(C(=O)O)cn3)CC2)CC1]

Referring to Scheme 17, 6-(4-(4-(tert-butoxycarbonyl)piperazin-1-yl)piperidin-1-yl)nicotinic acid (SIAIS631147) was prepared.

To a solution of 6-chloronicotinic acid (1 equiv.) in DMF (20 mL) were added DIPEA (5 equiv) and tert-butyl 4-(piperidin-4-yl)piperazine-1-carboxylate (2 equiv). The reaction mixture was reacted at 130° C. for 3 h, then was poured into 50 mL of water. The resulting mixture was extracted twice with dichloromethane. The organic phases were combined, washed with saturated brine, and concentrated under reduced pressure to remove the solvents. The resulting residue was purified by column chromatography (eluent (v/v): DCM to DCM/MeOH (10/1)) to give the intermediate compound SIAIS631147, ESI [M+H]⁺: 391.

### Step 2: Preparation of (E)-N-(4-(1-(6-(4-(piperazin-1-yl)piperidin-1-yl)nicotinoyl)piperidin-4-yl)butyl)-3-(pyridin-3-yl)acrylamide (SIAIS632044)

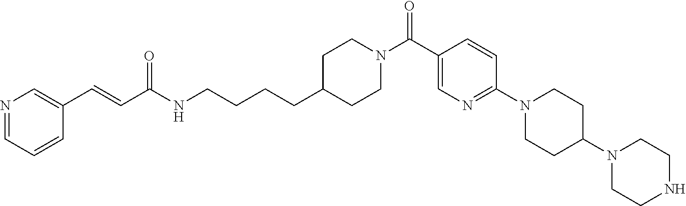 [SMILES: O=C(/C=C/c1cccnc1)NCCCCC1CCN(C(=O)c2ccc(N3CCC(N4CCNCC4)CC3)nc2)CC1]

Referring to Scheme 1 and using a method similar to the preparation of the Daporinad derivative 1 in Intermediate Preparation Example 1, the Daporinad derivative 7 ((E)-N-(4-(1-(6-(4-(piperazin-1-yl)piperidin-1-yl)nicotinoyl)piperidin-4-yl)butyl)-3-(pyridin-3-yl)acrylamide (SIAIS632044)) was prepared by using the intermediate SIAIS631147 from step 1. (white solid, 132 mg, yield 30%). ¹H NMR (500 MHz, CD₃OD) δ 9.04 (s, 1H), 8.80 (s, 1H), 8.70 (d, J=8.2 Hz, 1H), 8.13 (d, J=2.2 Hz, 1H), 8.04-7.94 (m, 1H), 7.86 (dd, J=9.2, 2.4 Hz, 1H), 7.63 (d, J=15.8 Hz, 1H), 7.23 (d, J=9.2 Hz, 1H), 6.91 (d, J=15.9 Hz, 1H), 4.48 (d, J=14.1 Hz, 4H), 3.54-3.49 (m, 6H), 3.34 (d, J=7.2 Hz, 2H), 3.26-3.16 (m, 3H), 2.25 (d, J=11.6 Hz, 4H), 1.87-1.76 (m, 5H), 1.62-1.58 (m, 4H), 1.46-1.41 (m, 2H), 1.38-1.28 (m, 2H), 1.23-1.13 (m, 2H). HRMS (ESI) calcd for C₃₂H₄₆N₇O₂⁺ [M+H]⁺: 560.3708, found, 560.3711.

### Intermediate Preparation Example 8: Preparation of 3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropanoic Acid (SIAIS352066)

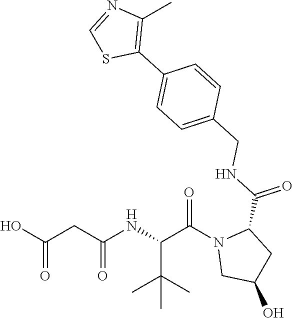 [SMILES: Cc1ncsc1-c1ccc(CNC(=O)[C@@H]2C[C@@H](O)CN2C(=O)[C@@H](NC(=O)CC(=O)O)C(C)(C)C)cc1]

Referring to the method of Scheme 14, the compound SIAIS352066 was prepared under appropriate conditions that will be recognized by one skilled in the art, except that the raw material diacid used was malonic acid. The compound (SIAIS352066) was obtained as white solid (0.82 g, yield 68%). ESI [M+H]⁺: 517.

### Intermediate Preparation Example 9: Preparation of 16-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-16-oxohexadecanoic Acid (SIAIS164189)

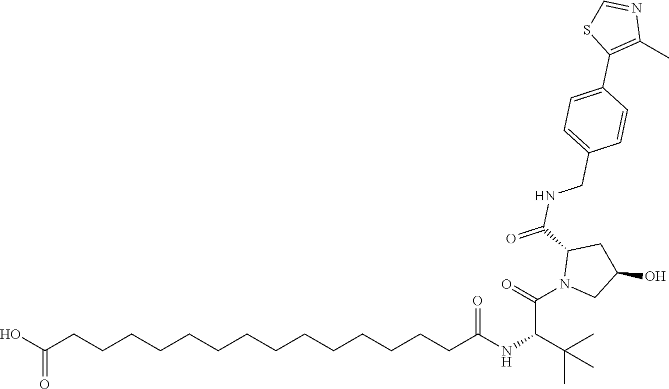 [SMILES: Cc1ncsc1-c1ccc(CNC(=O)[C@@H]2C[C@@H](O)CN2C(=O)[C@@H](NC(=O)CCCCCCCCCCCCCCC(=O)O)C(C)(C)C)cc1]

Referring to the method of Scheme 14, the compound SIAIS164189 was prepared under appropriate conditions that will be recognized by one skilled in the art, except that the raw material diacid used was hexadecanedioic acid. The compound (SIAIS164189) was obtained as white solid (0.88 g, yield 67%). ESI [M+H]⁺: 699.

### Intermediate Preparation Example 10: Preparation of 3-(4-((8-bromooctyl)oxy)-1-oxoisoindolin-2-yl)piperidine-2,6-dione (SIAIS1222063)

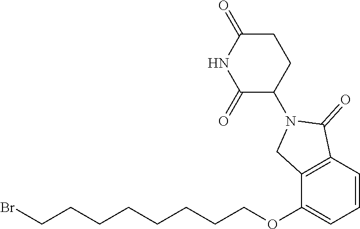 [SMILES: O=C1CCC(N2Cc3c(OCCCCCCCCBr)cccc3C2=O)C(=O)N1]

Referring to Scheme 12, to a solution of 3-(4-hydroxy-1-oxoisoindolin-2-yl)piperidine-2,6-dione (1.0 g, 3.8 mmol) in DMF (20 mL) was added K₂CO₃ (0.27 g, 1.9 mmol). The reaction mixture was stirred for 15 min, and the 1,8-dibromooctane (2.0 g, 7.6 mmol) was added thereto. The reaction solution was heated to 80° C. and reacted for 12 h, and then poured into 50 mL of water. The resulting mixture was extracted with dichloromethane twice. Organic phases were washed with saturated brine, and evaporated under reduced pressure to remove the solvent. The obtained residue was purified by column chromatography (eluent (v/v): DCM to DCM/MeOH (10/1)) to give the compound SIAIS1222063 as white powder (yield 32%). ESI [M+H]⁺: 451.

### Intermediate Preparation Example 11: Preparation of 4-((8-bromooctyl)thio)-2-(2,6-dioxopiperidin-3-yl)isoindoline-1,3-dione (SIAIS1224003)

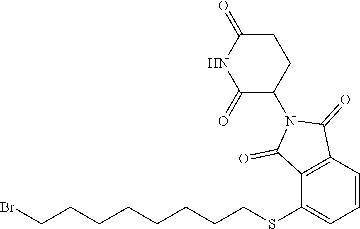 [SMILES: O=C1CCC(N2C(=O)c3cccc(SCCCCCCCCBr)c3C2=O)C(=O)N1]

Referring to the method of Scheme 11, the compound SIAIS1224003 was prepared under appropriate conditions that will be recognized by one skilled in the art, except that the raw materials used were 2-(2,6-dioxopiperidin-3-yl)-4-mercaptoisoindoline-1,3-dione and 1,8-dibromooctane. The compound (SIAIS1224003) was obtained as white solid (0.92 g, yield 71%). ESI [M+H]⁺: 481.

### Preparation Examples of the Compounds of the Present Invention

### Example 1: Preparation of (E)-N-(4-(1-(4-(4-(3-((2-(2,6-dioxopiperidin-3-yl)-1,3-dioxoisoindolin-4-yl)amino)propanoyl)piperazin-1-yl)benzoyl)piperidin-4-yl)butyl)-3-(pyridin-3-yl)acrylamide (SIAIS630009)

Referring to Scheme 15, a reaction flask was sequentially charged with Nampt inhibitor (i.e., Daporinad derivative 1 (SIAIS630006)) (0.02 mmol, 1 equiv), intermediate LM (SIAIS151026; 3-((2-(2,6-dioxopiperidin-3-yl)-1,3-dioxoisoindolin-4-yl)amino)propionic acid; CAS No.: 2225940-46-3) (0.02 mmol, 1 equiv), HATU (0.024 mmol, 1.2 equiv), 2 mL of DMF, and DIPEA (0.06 mmol, 3 equiv) at room temperature. The reaction mixture was reacted at room temperature overnight. Once the reaction was complete as monitored by LC-MS, the reaction mixture was filtered. The filtrate was subjected to preparative HPLC for separation. The collected fractions were evaporated to remove CH₃CN, and the resulting residue was lyophilized to afford the target compound (SIAIS630009) as yellow solid (6.3 mg, yield 46%). ¹H NMR (500 MHz, DMSO-d6) δ 11.09 (s, 1H), 9.00 (s, 1H), 8.80-8.75 (m, 1H), 8.48 (d, J=8.4 Hz, 1H), 8.33 (t, J=5.7 Hz, 1H), 7.88 (dd, J=8.2, 5.4 Hz, 1H), 7.62-7.49 (m, 2H), 7.26 (d, J=8.7 Hz, 2H), 7.16 (d, J=8.6 Hz, 1H), 7.03 (d, J=7.1 Hz, 1H), 6.96 (d, J=8.3 Hz, 2H), 6.89 (d, J=15.9 Hz, 1H), 6.79 (s, 1H), 5.04 (dd, J=12.8, 5.4 Hz, 1H), 3.67-3.50 (m, 9H), 3.30-3.13 (m, 6H), 2.92-2.83 (m, 2H), 2.72 (t, J=6.4 Hz, 2H), 2.63-2.55 (m, 1H), 2.49-2.44 (m, 1H), 2.01-1.96 (m, 1H), 1.67 (d, J=12.5 Hz, 2H), 1.49-1.42 (m, 2H), 1.36-1.18 (m, 4H), 1.09-1.00 (m, 2H). HRMS (ESI) calcd for C₄₄H₅₁N₈O₇⁺ [M+H]⁺, 803.3875; found, 803.3879.

### Example 2: Preparation of (E)-N-(4-(1-(4-(4-(4-((2-(2,6-dioxopiperidin-3-yl)-1,3-dioxoisoindolin-4-yl)amino)butanoyl)piperazin-1-yl)benzoyl)piperidin-4-yl)butyl)-3-(pyridin-3-yl)acrylamide (SIAIS630010)

Referring to the methods of example 1 and Scheme 15, the compound (SIAIS630010) was prepared by using Daporinad derivative 1 (SIAIS630006) and intermediate LM (SIAIS151019; 4-((2-(2,6-dioxopiperidin-3-yl)-1,3-dioxoisoindolin-4-yl)amino)butyric acid; CAS No.: 2225940-47-4). The compound (SIAIS630010) was obtained as yellow solid (6.8 mg, yield 49%). ¹H NMR (500 MHz, DMSO-d6) δ 11.09 (s, 1H), 8.99 (s, 1H), 8.76 (d, J=5.3 Hz, 1H), 8.46 (d, J=8.2 Hz, 1H), 8.37-8.27 (m, 1H), 7.86 (dd, J=8.2, 5.3 Hz, 1H), 7.58 (dd, J=8.6, 7.0 Hz, 1H), 7.53 (d, J=15.9 Hz, 1H), 7.26 (d, J=8.5 Hz, 2H), 7.18 (d, J=8.6 Hz, 1H), 7.01 (d, J=7.0 Hz, 1H), 6.98-6.94 (m, 2H), 6.88 (d, J=15.9 Hz, 1H), 6.67 (s, 1H), 5.05 (dd, J=12.7, 5.4 Hz, 1H), 3.63-3.57 (m, 6H), 3.34 (t, J=7.2 Hz, 3H), 3.27-3.14 (m, 6H), 2.93-2.85 (m, 2H), 2.63-2.53 (m, 1H), 2.54-2.50 (m, 1H), 2.46 (t, J=7.1 Hz, 2H), 2.07-1.97 (m, 1H), 1.82 (p, J=7.1 Hz, 2H), 1.66 (d, J=12.5 Hz, 2H), 1.53-1.41 (m, 3H), 1.31-1.20 (m, 4H), 1.11-1.00 (m, 2H). HRMS (ESI) calcd for C₄₅H₅₃N₈O₇⁺ [M+H]⁺, 817.4032; found, 817.4038.

### Example 3: Preparation of (E)-N-(4-(1-(4-(4-(5-((2-(2,6-dioxopiperidin-3-yl)-1,3-dioxoisoindolin-4-yl)amino)pentanoyl)piperazin-1-yl)benzoyl)piperidin-4-yl)butyl)-3-(pyridin-3-yl)acrylamide (SIAIS630011)

Referring to the methods of example 1 and Scheme 15, the compound (SIAIS630011) was prepared by using Daporinad derivative 1 (SIAIS630006) and intermediate LM (SIAIS151020; 5-((2-(2,6-dioxopiperidin-3-yl)-1,3-dioxoisoindolin-4-yl)amino)pentanoic acid; CAS No.: 2225940-48-5). The compound (SIAIS630011) was obtained as yellow solid (6.0 mg, yield 43%). ¹H NMR (500 MHz, CD₃OD) δ 8.85 (s, 1H), 8.63 (d, J=5.1 Hz, 1H), 8.36 (d, J=8.1 Hz, 1H), 7.77-7.70 (m, 1H), 7.63-7.48 (m, 2H), 7.31 (d, J=8.8 Hz, 2H), 7.07 (d, J=8.5 Hz, 1H), 6.99 (dd, J=17.8, 7.9 Hz, 3H), 6.81 (d, J=15.8 Hz, 1H), 5.03 (dd, J=12.6, 5.5 Hz, 1H), 3.70 (dt, J=16.9, 5.3 Hz, 4H), 3.41-3.35 (m, 2H), 3.35-3.33 (m, 2H), 3.24-3.21 (m, 6H), 2.88-2.81 (m, 1H), 2.77-2.71 (m, 1H), 2.52-2.51 (m, 2H), 2.13-2.02 (m, 1H), 1.75 (t, J=3.3 Hz, 5H), 1.59 (p, J=6.9 Hz, 4H), 1.43 (br, 2H), 1.37-1.32 (m, 2H), 1.29 (br, 2H), 0.18 (br, 2H). HRMS (ESI) calcd for C₄₆H₅₅N₈O₇⁺ [M+H]⁺, 831.4188; found, 831.4190.

### Example 4: Preparation of (E)-N-(4-(1-(4-(4-((2-(2,6-dioxopiperidin-3-yl)-1-oxoisoindolin-4-yl)glycyl)piperazin-1-yl)benzoyl)piperidin-4-yl)butyl)-3-(pyridin-3-yl)acrylamide (SIAIS632014)

Referring to the methods of example 1 and Scheme 15, the compound (SIAIS632014) was prepared by using Daporinad derivative 1 (SIAIS630006) and intermediate LM (SIAIS1204057; (2-(2,6-dioxopiperidin-3-yl)-1-oxoisoindolin-4-yl)aminoacetic acid; CAS No.: 2103656-92-2). The compound (SIAIS632014) was obtained as white solid (5.2 mg, yield 17%). ¹H NMR (500 MHz, CD₃OD) δ 8.96 (d, J=9.8 Hz, 1H), 8.73 (dd, J=7.5, 5.6 Hz, 1H), 8.60 (dd, J=23.2, 8.3 Hz, 1H), 7.96-7.90 (m, 1H), 7.61 (dd, J=15.8, 2.7 Hz, 1H), 7.38-7.26 (m, 3H), 7.12-6.98 (m, 3H), 6.88 (dd, J=15.8, 4.2 Hz, 2H), 5.17 (dd, J=13.3, 5.1 Hz, 1H), 4.39 (d, J=16.9 Hz, 1H), 4.35 (d, J=16.8 Hz, 1H), 3.78 (br, 2H), 3.54-3.47 (m, 2H), 3.40-3.32 (m, 12H), 2.97-2.86 (m, 1H), 2.83-2.77 (m, 1H), 2.54-2.44 (m, 1H), 2.25-2.17 (m, 1H), 1.75 (br, 1H), 1.58 (q, J=7.3 Hz, 3H), 1.43 (br, 2H), 1.37-1.25 (m, 3H), 1.16 (br, 2H). HRMS (ESI) calcd for C₄₃H₅₁N₈O₆⁺ [M+H]⁺, 775.3926; found, 775.3928.

### Example 5: Preparation of (E)-N-(4-(1-(4-(4-(4-((2-(2,6-dioxopiperidin-3-yl)-1-oxoisoindolin-4-yl)amino)butanoyl)piperazin-1-yl)benzoyl)piperidin-4-yl)butyl)-3-(pyridin-3-yl)acrylamide (SIAIS632015)

Referring to the methods of example 1 and Scheme 15, the compound (SIAIS632015) was prepared by using Daporinad derivative 1 (SIAIS630006) and intermediate LM (SIAIS1204085; 4-((2-(2,6-dioxopiperidin-3-yl)-1-oxoisoindolin-4-yl)amino)butyric acid; CAS No.: 1781226-49-0). The compound (SIAIS632015) was obtained as white solid (4.7 mg, yield 15%). ¹H NMR (500 MHz, CD₃OD) δ 9.00 (s, 1H), 8.77 (d, J=5.1 Hz, 1H), 8.69 (d, J=8.2 Hz, 1H), 8.00 (dd, J=8.2, 5.6 Hz, 1H), 7.64 (d, J=15.8 Hz, 1H), 7.37-7.29 (m, 3H), 7.10 (d, J=7.5 Hz, 1H), 6.99 (d, J=8.8 Hz, 2H), 6.95-6.89 (m, 2H), 5.18 (dd, J=13.3, 5.1 Hz, 1H), 4.36 (d, J=16.8 Hz, 1H), 4.30 (d, J=16.9 Hz, 1H), 3.71 (dt, J=32.4, 5.3 Hz, 4H), 3.39-3.35 (m, 7H), 3.24-3.17 (m, 5H), 3.03-2.89 (m, 1H), 2.84-2.79 (m, 1H), 2.61 (t, J=7.1 Hz, 2H), 2.56-2.40 (m, 1H), 2.24-2.17 (m, 1H), 2.02 (t, J=6.9 Hz, 2H), 1.79 (br, 1H), 1.61 (t, J=7.4 Hz, 4H), 1.48-1.45 (m, 2H), 1.39-1.34 (m, 2H), 1.18 (br, 2H). HRMS (ESI) calcd for C₄₅H₅₅N₈O₆⁺ [M+H]⁺, 803.4239; found, 803.4240.

### Example 6: Preparation of (E)-N-(4-(1-(4-(4-(5-((2-(2,6-dioxopiperidin-3-yl)-1-oxoisoindolin-4-yl)amino)pentanoyl)piperazin-1-yl)benzoyl)piperidin-4-yl)butyl)-3-(pyridin-3-yl)acrylamide (SIAIS632016)

Referring to the methods of example 1 and Scheme 15, the compound (SIAIS632016) was prepared by using Daporinad derivative 1 (SIAIS630006) and intermediate LM (SIAIS1210133; 5-((2-(2,6-dioxopiperidin-3-yl)-1-oxoisoindolin-4-yl)amino)pentanoic acid; CAS No.: 2338824-29-4). The compound (SIAIS632016) was obtained as white solid (4.1 mg, yield 14%). ¹H NMR (500 MHz, CD₃OD) δ 9.07 (d, J=2.0 Hz, 1H), 8.87-8.75 (m, 2H), 8.12 (dd, J=8.2, 5.8 Hz, 1H), 7.64 (d, J=15.8 Hz, 1H), 7.48 (t, J=7.8 Hz, 1H), 7.37 (dd, J=15.3, 8.1 Hz, 3H), 7.19 (d, J=7.9 Hz, 1H), 7.07 (d, J=8.8 Hz, 2H), 6.95 (d, J=15.8 Hz, 1H), 5.17 (dd, J=13.3, 5.1 Hz, 1H), 4.48 (d, J=17.0 Hz, 1H), 4.43 (d, J=17.1 Hz, 1H), 3.81-3.68 (m, 4H), 3.42-3.35 (m, 10H), 3.27 (t, J=5.2 Hz, 3H), 3.00-2.89 (m, 1H), 2.83-2.75 (m, 1H), 2.58-2.42 (m, 3H), 2.23-2.13 (m, 1H), 1.82-1.70 (m, 5H), 1.59 (q, J=7.3 Hz, 3H), 1.47-1.40 (m, 2H), 1.38-1.30 (m, 2H), 1.17 (br, 2H). HRMS (ESI) calcd for C₄₆H₅₇N₈O₆⁺ [M+H]⁺, 817.4396; found, 817.4398.

### Example 7: Preparation of (E)-N-(4-(1-(4-(4-(6-((2-(2,6-dioxopiperidin-3-yl)-1-oxoisoindolin-4-yl)amino)hexanoyl)piperazin-1-yl)benzoyl)piperidin-4-yl)butyl)-3-(pyridin-3-yl)acrylamide (SIAIS632017)

Referring to the methods of example 1 and Scheme 15, the compound (SIAIS632017) was prepared by using Daporinad derivative 1 (SIAIS630006) and intermediate LM (SIAIS1204061; 6-((2-(2,6-dioxopiperidin-3-yl)-1-oxoisoindolin-4-yl)amino)hexanoic acid; CAS No.: 2338824-30-7). The compound (SIAIS632017) was obtained as white solid (5.3 mg, yield 16%). ¹H NMR (500 MHz, DMSO-d₆) δ 11.01 (s, 1H), 9.00 (d, J=26.7 Hz, 1H), 8.79 (s, 1H), 8.53 (s, 1H), 8.33 (s, 1H), 7.92 (s, 1H), 7.54 (d, J=15.8 Hz, 1H), 7.32-7.23 (m, 3H), 7.00-6.75 (m, 5H), 5.11 (dd, J=13.3, 5.1 Hz, 1H), 4.26 (d, J=17.4 Hz, 1H), 4.16 (d, J=17.4 Hz, 1H), 3.58 (br, 6H), 3.28-3.07 (m, 9H), 2.95-2.86 (m, 3H), 2.65-2.58 (m, 1H), 2.36 (dd, J=8.3, 6.5 Hz, 2H), 2.31-2.22 (m, 1H), 2.07-1.95 (m, 1H), 1.71-1.52 (m, 5H), 1.49-1.35 (m, 5H), 1.32 (br, 2H), 1.25 (t, J=7.6 Hz, 2H), 1.11-0.99 (m, 2H). HRMS (ESI) calcd for C₄₇H₅₉N₈O₆⁺ [M+H]⁺, 831.4552; found, 831.4554.

### Example 8: Preparation of (E)-N-(4-(1-(4-(4-(7-((2-(2,6-dioxopiperidin-3-yl)-1-oxoisoindolin-4-yl)amino)heptanoyl)piperazin-1-yl)benzoyl)piperidin-4-yl)butyl)-3-(pyridin-3-yl)acrylamide (SIAIS632018)

Referring to the methods of example 1 and Scheme 15, the compound (SIAIS632018) was prepared by using Daporinad derivative 1 (SIAIS630006) and intermediate LM (SIAIS1204063; 7-((2-(2,6-dioxopiperidin-3-yl)-1-oxoisoindolin-4-yl)amino)heptanoic acid; CAS No.: 22338824-32-9). The compound (SIAIS632018) was obtained as white solid (5.7 mg, yield 17%). ¹H NMR (500 MHz, CD₃OD) δ 9.00 (s, 1H), 8.76 (d, J=5.6 Hz, 1H), 8.70 (dt, J=8.2, 1.8 Hz, 1H), 8.01 (dd, J=8.3, 5.6 Hz, 1H), 7.62 (d, J=15.8 Hz, 1H), 7.41-7.29 (m, 3H), 7.13 (d, J=7.5 Hz, 1H), 7.00 (d, J=8.8 Hz, 2H), 6.94-6.85 (m, 2H), 5.15 (dd, J=13.3, 5.1 Hz, 1H), 4.34 (d, J=16.9 Hz, 1H), 4.29 (d, J=16.9 Hz, 1H), 3.70 (dt, J=22.2, 5.3 Hz, 4H), 3.34 (d, J=7.0 Hz, 5H), 3.29-3.21 (m, 7H), 2.95-2.88 (m, 1H), 2.81-2.76 (m, 1H), 2.52-2.38 (m, 3H), 2.20-2.16 (m, 1H), 1.72-1.64 (m, 4H), 1.59 (p, J=7.4 Hz, 4H), 1.52-1.38 (m, 6H), 1.36-1.32 (m, 3H), 1.16 (br, 2H). HRMS (ESI) calcd for C₄₈H₆₁N₈O₆⁺ [M+H]⁺, 845.4709; found, 845.4711.

### Example 9: Preparation of (E)-N-(4-(1-(4-(4-(7-((2-(2,6-dioxopiperidin-3-yl)-1,3-dioxoisoindolin-4-yl)amino)heptanoyl)piperazin-1-yl)benzoyl)piperidin-4-yl)butyl)-3-(pyridin-3-yl)acrylamide (SIAIS630012)

Referring to the methods of example 1 and Scheme 15, the compound (SIAIS630012) was prepared by using Daporinad derivative 1 (SIAIS630006) and intermediate LM (SIAIS151086; 7-((2-(2,6-dioxopiperidin-3-yl)-1,3-dioxoisoindolin-4-yl)amino)heptanoic acid; CAS No.: 2225940-50-9). The compound (SIAIS630012) was obtained as yellow solid (6.1 mg, yield 44%). ¹H NMR (500 MHz, DMSO-d6) δ 11.09 (s, 1H), 8.96 (d, J=26.7 Hz, 1H), 8.74 (d, J=20.1 Hz, 1H), 8.39 (d, J=69.3 Hz, 2H), 7.82 (d, J=44.1 Hz, 1H), 7.58 (dd, J=8.6, 7.1 Hz, 1H), 7.52 (dd, J=15.9, 9.1 Hz, 1H), 7.26 (d, J=8.3 Hz, 2H), 7.10 (d, J=8.6 Hz, 1H), 7.02 (d, J=7.0 Hz, 1H), 6.96 (dd, J=9.2, 4.5 Hz, 2H), 6.92-6.80 (m, 1H), 6.54 (s, 1H), 5.05 (dd, J=12.8, 5.4 Hz, 1H), 3.62-3.53 (m, 6H), 3.30 (t, J=7.1 Hz, 3H), 3.25-3.15 (m, 6H), 2.93-2.85 (m, 2H), 2.62-2.54 (m, 1H), 2.53-2.52 (m, 1H), 2.35 (t, J=7.4 Hz, 2H), 2.07-1.98 (m, 1H), 1.72-1.63 (m, 2H), 1.61-1.43 (m, 7H), 1.39-1.30 (m, 6H), 1.25 (t, J=7.5 Hz, 2H), 1.10-0.99 (m, 2H). HRMS (ESI) calcd for C₄₈H₅₉N₈O₇⁺ [M+H]⁺, 859.4501; found, 859.4505.

### Example 10: Preparation of (E)-N-(4-(1-(4-(4-(2-((2-(2,6-dioxopiperidin-3-yl)-1-oxoisoindolin-4-yl)thio)acetyl)piperazin-1-yl)benzoyl)piperidin-4-yl)butyl)-3-(pyridin-3-yl)acrylamide (SIAIS630013)

Referring to the methods of example 1 and Scheme 15, the compound (SIAIS630013) was prepared by using Daporinad derivative 1 (SIAIS630006) and intermediate LM (SIAIS171090; 2-((2-(2,6-dioxopiperidin-3-yl)-1-oxoisoindolin-4-yl)thio)acetic acid; CAS No.: 2378582-40-0). The compound (SIAIS630013) was obtained as white solid (3.1 mg, yield 24%). ¹H NMR (500 MHz, CD₃OD) δ 8.94 (d, J=17.6 Hz, 1H), 8.73 (d, J=6.4 Hz, 1H), 8.61 (s, 1H), 7.93 (s, 1H), 7.80 (d, J=7.7 Hz, 1H), 7.73 (d, J=7.5 Hz, 1H), 7.66-7.52 (m, 2H), 7.32 (d, J=8.8 Hz, 2H), 6.99 (d, J=8.9 Hz, 2H), 6.87 (t, J=14.0 Hz, 1H), 5.16 (dd, J=13.4, 5.2 Hz, 1H), 4.56 (d, J=17.4 Hz, 1H), 4.49 (d, J=17.3 Hz, 1H), 4.00 (s, 2H), 3.69 (t, J=9.4 Hz, 5H), 3.35-3.33 (m, 3H), 3.22 (t, J=5.3 Hz, 6H), 2.95-2.85 (m, 1H), 2.83-2.72 (m, 1H), 2.54-2.42 (m, 1H), 2.21-2.15 (m, 1H), 1.77 (br, 2H), 1.67-1.50 (m, 5H), 1.47-1.40 (m, 2H), 1.39-1.27 (m, 2H), 1.18 (br, 2H). HRMS (ESI) calcd for C₄₃H₅₀N₇O₆S⁺ [M+H]⁺, 792.3538; found, 792.3540.

### Example 11: Preparation of (E)-N-(4-(1-(4-(4-(5-((2-(2,6-dioxopiperidin-3-yl)-1-oxoisoindolin-4-yl)thio)pentanoyl)piperazin-1-yl)benzoyl)piperidin-4-yl)butyl)-3-(pyridin-3-yl)acrylamide (SIAIS630014)

Referring to the methods of example 1 and Scheme 15, the compound (SIAIS630014) was prepared by using Daporinad derivative 1 (SIAIS630006) and intermediate LM (SIAIS171079; 5-((2-(2,6-dioxopiperidin-3-yl)-1-oxoisoindolin-4-yl)thio)pentanoic acid; CAS No.: 2378582-43-3). The compound (SIAIS630014) was obtained as white solid (4.0 mg, yield 29%). ¹H NMR (500 MHz, DMSO-d6) δ 10.98 (s, 1H), 8.92 (d, J=45.5 Hz, 1H), 8.70 (d, J=38.9 Hz, 1H), 8.50-8.18 (m, 2H), 7.82 (s, 1H), 7.64 (dd, J=7.5, 1.2 Hz, 1H), 7.59-7.46 (m, 3H), 7.26 (d, J=8.3 Hz, 2H), 6.99-6.92 (m, 2H), 6.89-6.76 (m, 1H), 5.12 (dd, J=13.3, 5.1 Hz, 1H), 4.36 (d, J=17.4 Hz, 1H), 4.22 (d, J=17.4 Hz, 1H), 3.60-3.55 (m, 10H), 3.24-3.16 (m, 5H), 3.11 (t, J=6.5 Hz, 2H), 2.95-2.85 (m, 2H), 2.65-2.54 (m, 1H), 2.49-2.44 (m, 1H), 2.41-2.36 (m, 2H), 2.05-1.95 (m, 1H), 1.68-1.62 (m, 5H), 1.52-1.40 (m, 2H), 1.35-1.20 (m, 4H), 1.13-0.96 (m, 2H). HRMS (ESI) calcd for C₄₆H₅₆N₇O₆S⁺ [M+H]⁺, 834.4007; found, 834.4011.

### Example 12: Preparation of (E)-N-(4-(1-(4-(4-(6-((2-(2,6-dioxopiperidin-3-yl)-1-oxoisoindolin-4-yl)thio)hexanoyl)piperazin-1-yl)benzoyl)piperidin-4-yl)butyl)-3-(pyridin-3-yl)acrylamide (SIAIS630037)

Referring to the methods of example 1 and Scheme 15, the compound (SIAIS6320037) was prepared by using Daporinad derivative 1 (SIAIS630006) and intermediate LM (SIAIS171091; 6-((2-(2,6-dioxopiperidin-3-yl)-1-oxoisoindolin-4-yl)thio)hexanoic acid; CAS No.: 2378582-44-4). The compound (SIAIS6320037) was obtained as white solid (4.6 mg, yield 33%). ¹H NMR (500 MHz, CD₃OD) δ 8.77 (s, 1H), 8.56 (d, J=5.1 Hz, 1H), 8.18 (d, J=8.1 Hz, 1H), 7.69-7.59 (m, 2H), 7.59-7.57 (m, 1H), 7.52 (dd, J=14.7, 7.1 Hz, 2H), 7.32 (d, J=8.6 Hz, 2H), 7.00 (d, J=8.7 Hz, 2H), 6.76 (d, J=15.8 Hz, 1H), 5.16 (dd, J=13.3, 5.2 Hz, 1H), 4.45 (d, J=17.3 Hz, 1H), 4.40 (d, J=17.3 Hz, 1H), 3.69 (dt, J=25.7, 5.2 Hz, 5H), 3.35 (br, 3H), 3.29-3.22 (m, 5H), 3.08 (td, J=7.1, 2.4 Hz, 3H), 2.95-2.85 (m, 1H), 2.82-2.74 (m, 1H), 2.43 (t, J=7.4 Hz, 2H), 2.21-2.10 (m, 1H), 1.72-1.55 (m, 10H), 1.45-1.40 (m, 2H), 1.36-1.30 (m, 4H), 1.16 (br, 2H). HRMS (ESI) calcd for C₄₇H₅₈N₇O₆S⁺ [M+H]⁺, 848.4164; found, 848.4168.

### Example 13: Preparation of (E)-N-(4-(1-(4-(4-(2-(2-((2-(2,6-dioxopiperidin-3-yl)-1-oxoisoindolin-4-yl)thio)ethoxy)acetyl)piperazin-1-yl)benzoyl)piperidin-4-yl)butyl)-3-(pyridin-3-yl)acrylamide (SIAIS630038)

Referring to the methods of example 1 and Scheme 15, the compound (SIAIS630038) was prepared by using Daporinad derivative 1 (SIAIS630006) and intermediate LM (2-(2-((2-(2,6-dioxopiperidin-3-yl)-1-oxoisoindolin-4-yl)thio)ethoxy)acetic acid; CAS No.: 2378582-21-7). The compound (SIAIS630038) was obtained as white solid (4.3 mg, yield 31%). ¹H NMR (500 MHz, CD₃OD) δ 8.84 (s, 1H), 8.63 (d, J=5.2 Hz, 1H), 8.34 (d, J=8.2 Hz, 1H), 7.74-7.69 (m, 2H), 7.64 (d, J=6.7 Hz, 1H), 7.58 (d, J=15.9 Hz, 1H), 7.51 (t, J=7.6 Hz, 1H), 7.32 (d, J=8.7 Hz, 2H), 6.97 (d, J=8.8 Hz, 2H), 6.81 (d, J=15.9 Hz, 1H), 5.14 (dd, J=13.4, 5.1 Hz, 1H), 4.49 (d, J=17.3 Hz, 1H), 4.43 (d, J=17.3 Hz, 1H), 4.23 (s, 2H), 3.74 (td, J=6.2, 2.1 Hz, 2H), 3.68 (br, 2H), 3.59 (t, J=5.3 Hz, 2H), 3.33 (d, J=7.2 Hz, 2H), 3.29 (d, J=6.2 Hz, 2H), 3.21 (br, 4H), 3.08 (br, 1H), 2.95-2.85 (m, 1H), 2.83-2.73 (m, 1H), 2.55-2.45 (m, 1H), 2.20-2.10 (m, 1H), 1.77 (br, 2H), 1.59 (p, J=7.1 Hz, 3H), 1.47-1.40 (m, 2H), 1.39-1.27 (m, 4H), 1.21-1.13 (m, 2H). HRMS (ESI) calcd for C₄₅H₅₄N₇O₇S⁺ [M+H]⁺, 836.3800; found, 836.3805.

### Example 14: Preparation of (E)-N-(4-(1-(4-(4-(2-(2-(2-((2-(2,6-dioxopiperidin-3-yl)-1-oxoisoindolin-4-yl)thio)ethoxy)ethoxy)acetyl)piperazin-1-yl)benzoyl)piperidin-4-yl)butyl)-3-(pyridin-3-yl)acrylamide (SIAIS630039)

Referring to the methods of example 1 and Scheme 15, the compound (SIAIS630039) was prepared by using Daporinad derivative 1 (SIAIS630006) and intermediate LM (SIAIS1213131; 2-(2-(2-((2-(2,6-dioxopiperidin-3-yl)-1-oxoisoindolin-4-yl)thio)ethoxy)ethoxy)acetic acid; CAS No.: 2378582-22-8). The compound (SIAIS630039) was obtained as white solid (4.2 mg, yield 31%). ¹H NMR (500 MHz, CD₃OD) δ 8.89 (s, 1H), 8.67 (s, 1H), 8.43 (d, J=8.1 Hz, 1H), 7.81-7.77 (m, 1H), 7.70-7.64 (m, 2H), 7.59 (d, J=15.9 Hz, 1H), 7.52 (t, J=7.7 Hz, 1H), 7.29 (d, J=8.9 Hz, 2H), 6.97 (d, J=8.9 Hz, 2H), 6.84 (d, J=15.8 Hz, 1H), 5.14 (dd, J=13.4, 5.2 Hz, 1H), 4.46 (d, J=17.4 Hz, 1H), 4.41 (d, J=17.3 Hz, 1H), 4.25 (s, 2H), 3.72-3.60 (m, 11H), 3.35-3.33 (m, 2H), 3.29-3.21 (m, 5H), 3.05 (br, 1H), 2.93-2.84 (m, 1H), 2.81-2.74 (m, 1H), 2.54-2.45 (m, 1H), 2.18-2.10 (m, 1H), 1.75 (br, 2H), 1.59 (p, J=7.1 Hz, 3H), 1.47-1.39 (m, 2H), 1.37-1.26 (m, 4H), 1.21-1.11 (m, 2H). HRMS (ESI) calcd for C₄₇H₅₈N₇O₈S⁺ [M+H]⁺, 880.4062; found, 880.4065.

### Example 15: Preparation of (E)-N-(4-(1-(4-(4-(2-((2-(2,6-dioxopiperidin-3-yl)-1-oxoisoindolin-4-yl)thio)ethyl)piperazin-1-yl)benzoyl)piperidin-4-yl)butyl)-3-(pyridin-3-yl)acrylamide (SIAIS630130)

Referring to Scheme 16, a reaction flask was sequentially charged with Nampt inhibitor (i.e., Daporinad derivative 1 (SIAIS630006)) (0.04 mmol, 1 equiv), intermediate LM (SIAIS213137; 3-(4-(2-bromoethylthio)-1-oxoisoindolin-2-yl)piperidine-2,6-dione; CAS No.: 2378582-57-9) (0.048 mmol, 1.2 equiv), 2 mL of DMF, and DIPEA (0.12 mmol, 3 equiv) at room temperature. The reaction mixture was reacted at 60° C. for 8 h. Once the reaction was complete as monitored by LC-MS, the reaction mixture was filtered. The filtrate was subjected to preparative HPLC for separation. The collected fractions were evaporated to remove CH₃CN, and the resulting residue was lyophilized to afford the target compound (SIAIS630130) as white solid (6.2 mg, yield 20%). ¹H NMR (500 MHz, CD₃OD) δ 8.92 (s, 1H), 8.70 (d, J=5.3 Hz, 1H), 8.50 (dt, J=8.2, 1.8 Hz, 1H), 7.92-7.74 (m, 3H), 7.67-7.56 (m, 2H), 7.35 (d, J=8.7 Hz, 2H), 7.06 (d, J=8.8 Hz, 2H), 6.86 (d, J=15.8 Hz, 1H), 5.19 (dd, J=13.5, 5.3 Hz, 1H), 4.56 (d, J=17.5 Hz, 1H), 4.49 (d, J=17.7 Hz, 1H), 3.81 (br, 1H), 3.72 (t, J=6.6 Hz, 1H), 3.54-3.40 (m, 6H), 3.33 (d, J=7.2 Hz, 4H), 3.17 (t, J=6.6 Hz, 1H), 2.98-2.85 (m, 1H), 2.84-2.75 (m, 1H), 2.60-2.56 (m, 1H), 2.25-2.15 (m, 1H), 1.76 (d, J=54.3 Hz, 2H), 1.65-1.54 (m, 4H), 1.45-1.27 (m, 1H), 1.38-1.27 (m, 2H), 1.15 (br, 2H). HRMS (ESI) calcd for C₄₃H₅₂N₇O₅S⁺ [M+H]⁺, 778.3745; found, 778.3748.

### Example 16: Preparation of (E)-N-(4-(1-(4-(4-(3-((2-(2,6-dioxopiperidin-3-yl)-1-oxoisoindolin-4-yl)thio)propyl)piperazin-1-yl)benzoyl)piperidin-4-yl)butyl)-3-(pyridin-3-yl)acrylamide (SIAIS630131)

Referring to the methods of example 15 and Scheme 16, the compound (SIAIS630131) was prepared by using Daporinad derivative 1 (SIAIS630006) and intermediate LM (SIAIS213132; 3-(4-(3-bromopropylthio)-1-oxoisoindolin-2-yl)piperidine-2,6-dione; CAS No.: 2378582-58-0). The compound (SIAIS630131) was obtained as white solid (6.4 mg, yield 21%). ¹H NMR (500 MHz, CD₃OD) δ 8.78 (d, J=2.1 Hz, 1H), 8.56 (d, J=3.9 Hz, 1H), 8.33 (d, J=8.2 Hz, 1H), 7.72-7.57 (m, 3H), 7.52-7.43 (m, 2H), 7.25 (d, J=8.8 Hz, 2H), 6.95 (d, J=8.9 Hz, 2H), 6.75 (d, J=15.9 Hz, 1H), 5.09 (dd, J=13.4, 5.2 Hz, 1H), 4.42 (d, J=17.4 Hz, 1H), 4.36 (d, J=17.4 Hz, 1H), 3.77 (d, J=65.1 Hz, 3H), 3.54 (br, 2H), 3.25 (dd, J=16.0, 7.7 Hz, 4H), 3.15-3.00 (m, 7H), 2.86-2.79 (m, 1H), 2.72-2.68 (m, 1H), 2.48-2.40 (m, 1H), 2.13-2.09 (m, 1H), 2.06-1.94 (m, 2H), 1.81-1.58 (m, 2H), 1.49 (p, J=7.1 Hz, 4H), 1.37-1.27 (m, 2H), 1.27-1.16 (m, 3H), 1.06 (br, 2H). HRMS (ESI) calcd for C₄₄H₅₄N₇O₅S⁺ [M+H]⁺, 792.3902; found, 792.3905.

### Example 17: Preparation of (E)-N-(4-(1-(4-(4-(4-((2-(2,6-dioxopiperidin-3-yl)-1-oxoisoindolin-4-yl)thio)butyl)piperazin-1-yl)benzoyl)piperidin-4-yl)butyl)-3-(pyridin-3-yl)acrylamide (SIAIS630123)

Referring to the methods of example 15 and Scheme 16, the compound (SIAIS630123) was prepared by using Daporinad derivative 1 (SIAIS630006) and intermediate LM (SIAIS213134; 3-(4-(4-bromobutylthio)-1-oxoisoindolin-2-yl)piperidine-2,6-dione; CAS No.: 2378582-59-1). The compound (SIAIS630123) was obtained as white solid (5.6 mg, yield 23%). ¹H NMR (500 MHz, CD₃OD) δ 8.88 (d, J=2.2 Hz, 1H), 8.66 (dd, J=5.3, 1.5 Hz, 1H), 8.39 (dt, J=8.1, 1.8 Hz, 1H), 7.79-7.65 (m, 3H), 7.63-7.55 (m, 2H), 7.37 (d, J=8.8 Hz, 2H), 7.08 (d, J=8.9 Hz, 2H), 6.85 (d, J=15.8 Hz, 1H), 5.20 (dd, J=13.4, 5.2 Hz, 1H), 4.52 (d, J=17.3 Hz, 1H), 4.45 (d, J=17.4 Hz, 1H), 3.90 (d, J=68.5 Hz, 3H), 3.65 (br, 2H), 3.35 (d, J=7.1 Hz, 4H), 3.26-3.11 (m, 9H), 2.98-2.91 (m, 1H), 2.84-2.79 (m, 1H), 2.60-2.51 (m, 1H), 2.24-2.19 (m, 1H), 2.01-1.94 (m, 2H), 1.84-1.73 (m, 3H), 1.61 (p, J=7.3 Hz, 3H), 1.48-1.42 (m, 2H), 1.40-1.28 (m, 3H), 1.18 (br, 2H). HRMS (ESI) calcd for C₄₅H₅₆N₇O₅S [M+H]⁺, 806.4058; found, 806.4061.

### Example 18: Preparation of (E)-N-(4-(1-(4-(4-(5-((2-(2,6-dioxopiperidin-3-yl)-1-oxoisoindolin-4-yl)thio)pentyl)piperazin-1-yl)benzoyl)piperidin-4-yl)butyl)-3-(pyridin-3-yl)acrylamide (SIAIS630132)

Referring to the methods of example 15 and Scheme 16, the compound (SIAIS630132) was prepared by using Daporinad derivative 1 (SIAIS630006) and intermediate LM (SIAIS1216049; 3-(4-((5-bromopentyl)thio)-1-oxoisoindolin-2-yl)piperidine-2,6-dione; CAS No.: 2378582-60-4). The compound (SIAIS630132) was obtained as white solid (6.1 mg, yield 19%). ¹H NMR (500 MHz, CD₃OD) δ 8.89 (d, J=2.1 Hz, 1H), 8.67 (dd, J=5.4, 1.5 Hz, 1H), 8.45 (dt, J=8.2, 1.8 Hz, 1H), 7.80 (dd, J=8.2, 5.3 Hz, 1H), 7.66 (ddd, J=7.5, 6.5, 1.0 Hz, 2H), 7.55 (dd, J=15.7, 8.1 Hz, 2H), 7.35 (d, J=8.8 Hz, 2H), 7.06 (d, J=8.9 Hz, 2H), 6.86 (d, J=15.9 Hz, 1H), 5.17 (dd, J=13.3, 5.1 Hz, 1H), 4.47 (d, J=17.3 Hz, 1H), 4.41 (d, J=17.4 Hz, 1H), 3.94-3.74 (m, 3H), 3.64 (br, 2H), 3.33 (d, J=7.1 Hz, 4H), 3.24-3.05 (m, 9H), 2.95-2.88 (m, 1H), 2.84-2.75 (m, 1H), 2.58-2.49 (m, 1H), 2.22-2.16 (m, 1H), 1.86-1.67 (m, 6H), 1.64-1.52 (m, 5H), 1.45-1.39 (m, 2H), 1.36-1.26 (m, 2H), 1.16 (br, 2H). HRMS (ESI) calcd for C₄₆H₅₈N₇O₅S⁺ [M+H]⁺, 820.4215; found, 820.4217.

### Example 19: Preparation of (E)-N-(4-(1-(4-(4-(6-((2-(2,6-dioxopiperidin-3-yl)-1-oxoisoindolin-4-yl)thio)hexyl)piperazin-1-yl)benzoyl)piperidin-4-yl)butyl)-3-(pyridin-3-yl)acrylamide (SIAIS630133)

Referring to the methods of example 15 and Scheme 16, the compound (SIAIS630133) was prepared by using Daporinad derivative 1 (SIAIS630006) and intermediate LM (SIAIS1216133; 3-(4-((6-bromohexyl)thio)-1-oxoisoindolin-2-yl)piperidine-2,6-dione; CAS No.: 2378582-61-5). The compound (SIAIS630133) was obtained as white solid (6.4 mg, yield 19%). ¹H NMR (500 MHz, CD₃OD) δ 8.88 (d, J=2.1 Hz, 1H), 8.65 (dd, J=5.3, 1.5 Hz, 1H), 8.41 (dt, J=8.1, 1.8 Hz, 1H), 7.77 (dd, J=8.1, 5.3 Hz, 1H), 7.67-7.63 (m, 2H), 7.61-7.52 (m, 2H), 7.35 (d, J=8.8 Hz, 2H), 7.06 (d, J=8.8 Hz, 2H), 6.85 (d, J=15.9 Hz, 1H), 5.17 (dd, J=13.3, 5.2 Hz, 1H), 4.47 (d, J=17.3 Hz, 1H), 4.41 (d, J=17.3 Hz, 1H), 3.98-3.77 (m, 3H), 3.64 (br, 2H), 3.33 (d, J=7.0 Hz, 3H), 3.19-3.03 (m, 10H), 2.95-2.86 (m, 1H), 2.82-2.77 (m, 1H), 2.58-2.49 (m, 1H), 2.21-2.16 (m, 1H), 1.81-1.67 (m, 6H), 1.62-1.51 (m, 5H), 1.46-1.38 (m, 4H), 1.36-1.30 (m, 2H), 1.16 (br, 2H). HRMS (ESI) calcd for C₄₇H₆₀N₇O₅S⁺ [M+H]⁺, 834.4371; found, 834.4375.

### Example 20: Preparation of (E)-N-(4-(1-(4-(4-(7-((2-(2,6-dioxopiperidin-3-yl)-1-oxoisoindolin-4-yl)thio)heptyl)piperazin-1-yl)benzoyl)piperidin-4-yl)butyl)-3-(pyridin-3-yl)acrylamide (SIAIS630134)

Referring to the methods of example 15 and Scheme 16, the compound (SIAIS630134) was prepared by using Daporinad derivative 1 (SIAIS630006) and intermediate LM (SIAIS1216135; 3-(4-(7-bromoheptylthio)-1-oxoisoindolin-2-yl)piperidine-2,6-dione; CAS No.: 2378582-62-6). The compound (SIAIS630134) was obtained as white solid (6.7 mg, yield 20%). ¹H NMR (500 MHz, CD₃OD) δ 8.86 (d, J=2.2 Hz, 1H), 8.64 (dd, J=5.3, 1.5 Hz, 1H), 8.39 (dt, J=8.2, 1.8 Hz, 1H), 7.75 (dd, J=8.1, 5.3 Hz, 1H), 7.65 (dt, J=7.0, 1.3 Hz, 2H), 7.62-7.47 (m, 2H), 7.35 (d, J=8.8 Hz, 2H), 7.06 (d, J=8.9 Hz, 2H), 6.84 (d, J=15.8 Hz, 1H), 5.16 (dd, J=13.3, 5.2 Hz, 1H), 4.46 (d, J=17.3 Hz, 1H), 4.40 (d, J=17.3 Hz, 1H), 3.98-3.79 (m, 3H), 3.64 (br, 2H), 3.33 (d, J=7.1 Hz, 3H), 3.26-3.00 (m, 10H), 2.93-2.87 (m, 1H), 2.82-2.76 (m, 1H), 2.58-2.49 (m, 1H), 2.21-2.16 (m, 1H), 1.82-1.74 (m, 3H), 1.72-1.65 (m, 2H), 1.61-1.56 (m, 3H), 1.51-1.47 (m, 2H), 1.46-1.28 (m, 9H), 1.16 (br, 2H). HRMS (ESI) calcd for C₄₈H₆₂N₇O₅S⁺ [M+H]⁺, 848.4528; found, 848.4531.

### Example 21: Preparation of (E)-N-(4-(1-(4-(4-(8-((2-(2,6-dioxopiperidin-3-yl)-1-oxoisoindolin-4-yl)thio)octyl)piperazin-1-yl)benzoyl)piperidin-4-yl)butyl)-3-(pyridin-3-yl)acrylamide (SIAIS630135)

Referring to the methods of example 15 and Scheme 16, the compound (SIAIS630135) was prepared by using Daporinad derivative 1 (SIAIS630006) and intermediate LM (SIAIS1216137; 3-(4-((8-bromooctyl)thio)-1-oxoisoindolin-2-yl)piperidine-2,6-dione; CAS No.: 2378582-63-7). The compound (SIAIS630135) was obtained as white solid (6.9 mg, yield 20%). ¹H NMR (500 MHz, CD₃OD) δ 8.84 (d, J=14.4 Hz, 1H), 8.63 (dd, J=11.6, 5.1 Hz, 1H), 8.40-8.29 (m, 1H), 7.77-7.68 (m, 1H), 7.69-7.62 (m, 2H), 7.62-7.50 (m, 2H), 7.36 (d, J=8.8 Hz, 2H), 7.07 (d, J=8.9 Hz, 2H), 6.82 (t, J=14.1 Hz, 1H), 5.17 (dd, J=13.3, 5.2 Hz, 1H), 4.46 (d, J=17.3 Hz, 1H), 4.40 (d, J=17.3 Hz, 1H), 3.96 (d, J=13.4 Hz, 2H), 3.65 (d, J=12.0 Hz, 2H), 3.33 (d, J=5.1 Hz, 6H), 3.24-3.02 (m, 8H), 2.95-2.85 (m, 1H), 2.83-2.75 (m, 1H), 2.58-2.49 (m, 1H), 2.21-2.16 (m, 1H), 1.75 (br, 3H), 1.68 (q, J=7.4 Hz, 2H), 1.59 (p, J=7.3 Hz, 3H), 1.53-1.28 (m, 12H), 1.18 (br, 2H). HRMS (ESI) calcd for C₄₉H₆₄N₇O₅S⁺ [M+H]⁺, 862.4684; found, 862.4689.

### Example 22: Preparation of (E)-N-(4-(1-(4-(4-(11-((2-(2,6-dioxopiperidin-3-yl)-1-oxoisoindolin-4-yl)thio)undecyl)piperazin-1-yl)benzoyl)piperidin-4-yl)butyl)-3-(pyridin-3-yl)acrylamide (SIAIS630136)

Referring to the methods of example 15 and Scheme 16, the compound (SIAIS630136) was prepared by using Daporinad derivative 1 (SIAIS630006) and intermediate LM (SIAIS1220015; 3-(4-((11-bromoundecyl)thio)-1-oxoisoindolin-2-yl)piperidine-2,6-dione; CAS No.: 2378582-66-0). The compound (SIAIS630136) was obtained as white solid (7.0 mg, yield 20%). ¹H NMR (500 MHz, CD₃OD) δ 8.83 (d, J=2.3 Hz, 1H), 8.62 (dd, J=5.2, 1.6 Hz, 1H), 8.33 (dt, J=8.2, 1.8 Hz, 1H), 7.70 (dd, J=8.1, 5.2 Hz, 1H), 7.66-7.59 (m, 2H), 7.59-7.50 (m, 2H), 7.35 (d, J=8.8 Hz, 2H), 7.06 (d, J=8.8 Hz, 2H), 5.15 (dd, J=13.3, 5.2 Hz, 1H), 4.44 (d, J=17.3 Hz, 1H), 4.39 (d, J=17.3 Hz, 1H), 3.95-3.78 (m, 3H), 3.67 (br, 2H), 3.33 (d, J=7.1 Hz, 3H), 3.24-3.02 (m, 10H), 2.94-2.86 (m, 1H), 2.81-2.76 (m, 1H), 2.57-2.48 (m, 1H), 2.21-2.15 (m, 1H), 1.84-1.72 (m, 3H), 1.69-1.63 (m, 2H), 1.60-1.55 (m, 2H), 1.50-1.24 (m, 20H), 1.15 (br, 3H). HRMS (ESI) calcd for C₅₂H₇₀N₇O₅S⁺ [M+H]⁺, 904.5154; found, 904.5155.

### Example 23: Preparation of (E)-N-(4-(1-(4-(4-(5-(2-(2,6-dioxopiperidin-3-yl)-1-oxoisoindolin-4-yl)pent-4-yn-1-yl)piperazin-1-yl)benzoyl)piperidin-4-yl)butyl)-3-(pyridin-3-yl)acrylamide (SIAIS630129)

Referring to the methods of example 15 and Scheme 16, the compound (SIAIS630129) was prepared by using Daporinad derivative 1 (SIAIS630006) and intermediate LM (SIAIS255121; 5-(2-(2,6-dioxopiperidin-3-yl)-1-oxoisoindolin-4-yl)pent-4-yn-1-yl methanesulfonate; CAS No.: 2138441-31-1). The compound (SIAIS630129) was obtained as white solid (5.3 mg, yield 17%). ¹H NMR (500 MHz, CD₃OD) δ 8.75 (d, J=2.1 Hz, 1H), 8.53 (dd, J=5.2, 1.5 Hz, 1H), 8.25 (d, J=8.3 Hz, 1H), 7.68 (d, J=7.7 Hz, 1H), 7.63 (dd, J=8.1, 5.2 Hz, 1H), 7.57 (dd, J=7.8, 1.0 Hz, 1H), 7.48 (d, J=15.9 Hz, 1H), 7.43 (t, J=7.7 Hz, 1H), 7.26 (d, J=8.8 Hz, 2H), 6.98 (d, J=8.8 Hz, 2H), 6.72 (d, J=15.9 Hz, 1H), 5.10 (dd, J=13.3, 5.2 Hz, 1H), 4.48 (d, J=17.5 Hz, 1H), 4.41 (d, J=17.5 Hz, 1H), 3.88 (br, 2H), 3.61 (br, 3H), 3.33-3.25 (m, 2H), 3.24-3.22 (m, 5H), 3.14-2.99 (m, 2H), 2.86-2.79 (m, 1H), 2.72-2.67 (m, 1H), 2.60 (t, J=6.9 Hz, 2H), 2.48-2.40 (m, 1H), 2.14-1.99 (m, 3H), 1.67 (d, J=53.2 Hz, 2H), 1.52-1.46 (m, 3H), 1.38-1.27 (m, 2H), 1.28-1.18 (m, 3H), 1.07 (br, 3H). HRMS (ESI) calcd for C₄₆H₅₄N₇O₅⁺ [M+H]⁺, 784.4181; found, 784.4183.

### Example 24: Preparation of (E)-N-(4-(1-(4-(4-(6-(2-(2,6-dioxopiperidin-3-yl)-1-oxoisoindolin-4-yl)hex-5-yn-1-yl)piperazin-1-yl)benzoyl)piperidin-4-yl)butyl)-3-(pyridin-3-yl)acrylamide (SIAIS630119)

Referring to the methods of example 15 and Scheme 16, the compound (SIAIS630119) was prepared by using Daporinad derivative 1 (SIAIS630006) and intermediate LM (SIAIS255119; 6-(2-(2,6-dioxopiperidin-3-yl)-1-oxoisoindolin-4-yl)hex-5-yn-1-yl methanesulfonate; CAS No.: 2570254-40-7). The compound (SIAIS630119) was obtained as white solid (5.6 mg, yield 24%). ¹H NMR (500 MHz, CD₃OD) δ 8.88 (s, 1H), 8.70-8.61 (m, 1H), 8.41 (d, J=8.2 Hz, 1H), 7.81-7.74 (m, 2H), 7.68-7.55 (m, 2H), 7.51 (t, J=7.6 Hz, 1H), 7.35 (d, J=8.7 Hz, 2H), 7.07 (d, J=8.9 Hz, 2H), 6.84 (d, J=15.9 Hz, 1H), 5.19 (dd, J=13.3, 5.2 Hz, 1H), 4.56 (d, J=17.5 Hz, 1H), 4.50 (d, J=17.5 Hz, 1H), 3.95 (br, 1H), 3.70 (br, 3H), 3.33 (d, J=7.1 Hz, 5H), 3.29-3.26 (m, 7H), 2.95-2.88 (m, 1H), 2.81-2.76 (m, 1H), 2.63 (t, J=6.9 Hz, 2H), 2.57-2.50 (m, 1H), 2.25-2.15 (m, 1H), 2.03-1.97 (m, 2H), 1.85-1.70 (m, 4H), 1.59 (p, J=7.1 Hz, 3H), 1.45-1.39 (m, 2H), 1.36-1.31 (m, 2H), 1.16 (br, 2H). HRMS (ESI) calcd for C₄₇H₅₆N₇O₅⁺ [M+H]⁺, 798.4337; found, 798.4339.

### Example 25: Preparation of (E)-N-(4-(1-(4-(4-(7-(2-(2,6-dioxopiperidin-3-yl)-1-oxoisoindolin-4-yl)hept-6-yn-1-yl)piperazin-1-yl)benzoyl)piperidin-4-yl)butyl)-3-(pyridin-3-yl)acrylamide (SIAIS630120)

Referring to the methods of example 15 and Scheme 16, the compound (SIAIS630120) was prepared by using Daporinad derivative 1 (SIAIS630006) and intermediate LM (SIAIS292017; 7-(2-(2,6-dioxopiperidin-3-yl)-1-oxoisoindolin-4-yl)hept-6-yn-1-yl methanesulfonate; CAS No.: 2570254-41-8). The compound (SIAIS630120) was obtained as white solid (5.8 mg, yield 25%). ¹H NMR (500 MHz, DMSO-d₆) δ 11.00 (s, 1H), 8.89 (s, 1H), 8.72-8.61 (m, 1H), 8.26 (t, J=5.3 Hz, 2H), 7.71 (t, J=8.2 Hz, 2H), 7.65 (d, J=7.5 Hz, 1H), 7.57-7.46 (m, 2H), 7.28 (d, J=8.5 Hz, 2H), 7.01 (d, J=8.4 Hz, 2H), 6.82 (d, J=15.9 Hz, 2H), 5.16 (dd, J=13.3, 5.1 Hz, 1H), 4.47 (d, J=17.7 Hz, 1H), 4.33 (d, J=17.7 Hz, 1H), 3.89 (d, J=13.0 Hz, 2H), 3.55 (br, 4H), 3.25-3.15 (m, 3H), 3.15-3.05 (m, 5H), 2.95-2.87 (m, 1H), 2.68-2.57 (m, 1H), 2.56-2.53 (m, 1H), 2.47-2.39 (m, 1H), 2.06-1.97 (m, 1H), 1.84-1.75 (m, 2H), 1.72-1.60 (m, 4H), 1.52-1.43 (m, 5H), 1.36-1.26 (m, 2H), 1.25-1.20 (m, 2H), 1.09-0.98 (m, 2H). HRMS (ESI) calcd for C₄₈H₅₈N₇O₅⁺ [M+H]⁺, 812.4494; found, 812.4496.

### Example 26: Preparation of (E)-N-(4-(1-(4-(4-(8-(2-(2,6-dioxopiperidin-3-yl)-1-oxoisoindolin-4-yl)oct-7-yn-1-yl)piperazin-1-yl)benzoyl)piperidin-4-yl)butyl)-3-(pyridin-3-yl)acrylamide (SIAIS630121)

Referring to the methods of example 15 and Scheme 16, the compound (SIAIS630121) was prepared by using Daporinad derivative 1 (SIAIS630006) and intermediate LM (SIAIS292020; 8-(2-(2,6-dioxopiperidin-3-yl)-1-oxoisoindolin-4-yl)oct-7-yn-1-yl methanesulfonate; CAS No.: 2570254-42-9). The compound (SIAIS630121) was obtained as white solid (5.7 mg, yield 24%). ¹H NMR (500 MHz, CD₃OD) δ 8.84 (d, J=2.2 Hz, 1H), 8.63 (dd, J=5.2, 1.5 Hz, 1H), 8.37-8.32 (m, 1H), 7.75 (d, J=6.6 Hz, 1H), 7.71 (dd, J=8.1, 5.2 Hz, 1H), 7.63-7.55 (m, 2H), 7.51 (t, J=7.6 Hz, 1H), 7.36 (d, J=8.7 Hz, 2H), 7.07 (d, J=8.9 Hz, 2H), 6.81 (d, J=15.9 Hz, 1H), 5.19 (dd, J=13.3, 5.2 Hz, 1H), 4.53 (d, J=17.4 Hz, 1H), 4.47 (d, J=17.4 Hz, 1H), 3.95 (br, 1H), 3.67 (br, 2H), 3.39-3.32 (m, 6H), 3.25-3.19 (m, 4H), 3.18-3.11 (m, 2H), 2.95-2.86 (m, 1H), 2.84-2.75 (m, 1H), 2.57-2.49 (m, 3H), 2.24-2.15 (m, 1H), 1.86-1.80 (m, 2H), 1.70 (p, J=6.9 Hz, 3H), 1.65-1.55 (m, 5H), 1.53-1.39 (m, 4H), 1.37-1.27 (m, 3H), 1.24-1.07 (m, 2H). HRMS (ESI) calcd for C₄₉H₆₀N₇O₅⁺ [M+H]⁺, 826.4650; found, 826.4655.

### Example 27: Preparation of (E)-N-(4-(1-(4-(4-(9-(2-(2,6-dioxopiperidin-3-yl)-1-oxoisoindolin-4-yl)non-8-yn-1-yl)piperazin-1-yl)benzoyl)piperidin-4-yl)butyl)-3-(pyridin-3-yl)acrylamide (SIAIS630122)

Referring to the methods of example 15 and Scheme 16, the compound (SIAIS630122) was prepared by using Daporinad derivative 1 (SIAIS630006) and intermediate LM (SIAIS255127; 9-(2-(2,6-dioxopiperidin-3-yl)-1-oxoisoindolin-4-yl)non-8-yn-1-yl methanesulfonate; CAS No.: 2600734-35-6). The compound (SIAIS630122) was obtained as white solid (6.3 mg, yield 25%). ¹H NMR (500 MHz, CD₃OD) δ 8.75 (s, 1H), 8.55 (s, 1H), 8.13 (d, J=8.0 Hz, 1H), 7.75 (dd, J=7.6, 1.0 Hz, 1H), 7.61 (dd, J=7.7, 1.0 Hz, 1H), 7.58-7.49 (m, 3H), 7.36 (dd, J=8.8, 1.8 Hz, 2H), 7.06 (dd, J=9.0, 2.5 Hz, 2H), 6.75 (d, J=15.9 Hz, 1H), 5.19 (dd, J=13.4, 5.2 Hz, 1H), 4.53 (d, J=17.4 Hz, 1H), 4.46 (d, J=17.5 Hz, 1H), 3.94 (d, J=13.6 Hz, 2H), 3.83 (br, 1H), 3.64 (d, J=11.9 Hz, 2H), 3.35-3.32 (m, 2H), 3.25-3.16 (m, 4H), 3.11 (br, 2H), 2.98 (s, 2H), 2.96-2.86 (m, 2H), 2.84-2.76 (m, 1H), 2.61-2.48 (m, 2H), 2.25-2.16 (m, 1H), 1.83-1.75 (m, 3H), 1.67 (p, J=6.9 Hz, 3H), 1.62-1.53 (m, 4H), 1.50-1.38 (m, 6H), 1.37-1.27 (m, 4H), 1.21-1.13 (m, 2H). HRMS (ESI) calcd for C₅₀H₆₂N₇O₅⁺ [M+H]⁺, 840.4807; found, 840.4809.

### Example 28: Preparation of (2S,4R)-1-((S)-3,3-dimethyl-2-(3-oxo-3-(4-(4-(4-(4-((E)-3-(pyridin-3-yl)acrylamido)butyl)piperidine-1-carbonyl)phenyl)piperazin-1-yl)propanamido)butanoyl)-4-hydroxy-N-(4-(4-methylthiazol-5-yl)benzyl)pyrrolidine-2-carboxamide (SIAIS631001)

Referring to the methods of example 1 and Scheme 15, the compound (SIAIS631001) was prepared by using Daporinad derivative 1 (SIAIS630006) and intermediate LM (SIAIS352066). The compound (SIAIS631001) was obtained as white solid (11.9 mg, yield 31%). ¹H NMR (500 MHz, DMSO-d6) δ 9.05 (d, J=6.4 Hz, 2H), 8.82 (d, J=4.2 Hz, 1H), 8.61 (d, J=6.2 Hz, 2H), 8.39 (t, J=5.5 Hz, 1H), 8.25 (d, J=9.4 Hz, 1H), 8.03-7.94 (m, 1H), 7.55 (d, J=15.9 Hz, 1H), 7.41 (q, J=8.4 Hz, 4H), 7.27 (dd, J=8.6, 5.2 Hz, 2H), 7.02-6.91 (m, 3H), 4.54 (d, J=9.4 Hz, 1H), 4.47-4.39 (m, 3H), 4.37-4.34 (m, 1H), 4.25-4.19 (m, 1H), 3.68 (dd, J=10.6, 4.1 Hz, 2H), 3.64-3.57 (m, 5H), 3.54 (d, J=15.5 Hz, 1H), 3.49-3.42 (m, 2H), 3.30-3.14 (m, 6H), 3.13-3.07 (m, 1H), 2.94-2.67 (m, 2H), 2.45 (s, 3H), 2.07-2.00 (m, 1H), 1.95-1.85 (m 1H), 1.70-1.64 (m, 1H), 1.46 (p, J=7.1 Hz, 3H), 1.35-1.28 (m, 2H), 1.29-1.22 (m, 2H), 1.09-0.99 (m, 2H), 0.95 (s, 9H). HRMS (ESI) calcd for C₅₃H₆₈N₉O₇S⁺ [M+H]⁺, 974.4957; found, 974.4959.

### Example 29: Preparation of (2S,4R)-1-((S)-3,3-dimethyl-2-(4-oxo-4-(4-(4-(4-(4-((E)-3-(pyridin-3-yl)acrylamido)butyl)piperidine-1-carbonyl)phenyl)piperazin-1-yl)butanamido)butanoyl)-4-hydroxy-N-(4-(4-methylthiazol-5-yl)benzyl)pyrrolidine-2-carboxamide (SIAIS631002)

Referring to the methods of example 1 and Scheme 15, the compound (SIAIS631002) was prepared by using Daporinad derivative 1 (SIAIS630006) and intermediate LM (SIAIS074011; 4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-4-oxobutanoic acid; CAS No.: 2172819-72-4). The compound (SIAIS631002) was obtained as white solid (11.5 mg, yield 30%). ¹H NMR (500 MHz, DMSO-d6) δ 9.08 (d, J=5.9 Hz, 2H), 8.84 (d, J=4.3 Hz, 1H), 8.65 (d, J=8.4 Hz, 1H), 8.59 (t, J=6.1 Hz, 1H), 8.41 (t, J=5.5 Hz, 1H), 8.02 (dd, J=8.1, 5.5 Hz, 1H), 7.93 (d, J=9.3 Hz, 1H), 7.56 (d, J=15.9 Hz, 1H), 7.41 (q, J=8.3 Hz, 4H), 7.30-7.23 (m, 2H), 7.06-6.92 (m, 3H), 4.52 (d, J=9.3 Hz, 1H), 4.46-4.38 (m, 3H), 4.36-4.33 (m, 1H), 4.26-4.18 (m, 1H), 3.71-3.57 (m, 6H), 3.45 (t, J=5.3 Hz, 1H), 3.28 (t, J=5.3 Hz, 1H), 3.19 (p, J=6.6, 6.0 Hz, 5H), 2.65-2.53 (m, 3H), 2.45 (s, 3H), 2.44-2.35 (m, 2H), 2.06-2.00 (m, 1H), 1.95-1.85 (m, 1H), 1.66 (d, J=12.4 Hz, 2H), 1.51-1.42 (m, 3H), 1.36-1.28 (m, 2H), 1.26-1.21 (m, 2H), 1.09-1.00 (m, 2H), 0.93 (s, 9H). HRMS (ESI) calcd for C₅₄H₇₀N₉O₇S⁺ [M+H]⁺, 988.5113; found, 988.5116.

### Example 30: Preparation of (2S,4R)-1-((S)-3,3-dimethyl-2-(5-oxo-5-(4-(4-(4-(4-((E)-3-(pyridin-3-yl)acrylamido)butyl)piperidine-1-carbonyl)phenyl)piperazin-1-yl)pentanamido)butanoyl)-4-hydroxy-N-(4-(4-methylthiazol-5-yl)benzyl)pyrrolidine-2-carboxamide (SIAIS631003)

Referring to the methods of example 1 and Scheme 15, the compound (SIAIS631003) was prepared by using Daporinad derivative 1 (SIAIS630006) and intermediate LM (SIAIS074012; 5-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-5-oxopentanoic acid; CAS No.: 2172819-73-5). The compound (SIAIS631003) was obtained as white solid (12.8 mg, yield 33%). ¹H NMR (500 MHz, CD₃OD) δ 9.66 (d, J=19.2 Hz, 1H), 9.08 (d, J=2.0 Hz, 1H), 8.86-8.77 (m, 2H), 8.13 (dd, J=8.2, 5.8 Hz, 1H), 7.64 (d, J=15.9 Hz, 1H), 7.57-7.46 (m, 4H), 7.36 (d, J=8.9 Hz, 2H), 7.12 (d, J=8.3 Hz, 1H), 7.08 (d, J=8.8 Hz, 1H), 6.97 (dd, J=15.9, 1.0 Hz, 1H), 4.62 (d, J=1.8 Hz, 1H), 4.59-4.52 (m, 2H), 4.52-4.47 (m, 1H), 4.38 (dd, J=15.7, 3.6 Hz, 1H), 3.97-3.88 (m, 1H), 3.84-3.72 (m, 3H), 3.54-3.48 (m, 3H), 3.41-3.32 (m, 12H), 2.57 (s, 3H), 2.48 (t, J=7.5 Hz, 1H), 2.40-2.34 (m, 2H), 2.34-2.28 (m, 1H), 2.26-2.20 (m, 1H), 2.11-2.04 (m, 1H), 1.91 (dt, J=17.0, 7.5 Hz, 2H), 1.60 (p, J=6.8 Hz, 3H), 1.47-1.29 (m, 5H), 1.21-1.11 (m, 2H), 1.04 (s, 9H). HRMS (ESI) calcd for C₅₅H₇₂N₉O₇S⁺ [M+H]⁺, 1002.5270; found, 1002.5273.

### Example 31: Preparation of (2S,4R)-1-((S)-3,3-dimethyl-2-(6-oxo-6-(4-(4-(4-(4-((E)-3-(pyridin-3-yl)acrylamido)butyl)piperidine-1-carbonyl)phenyl)piperazin-1-yl)hexanamido)butanoyl)-4-hydroxy-N-(4-(4-methylthiazol-5-yl)benzyl)pyrrolidine-2-carboxamide (SIAIS630007)

Referring to the methods of example 1 and Scheme 15, the compound (SIAIS630007) was prepared by using Daporinad derivative 1 (SIAIS630006) and intermediate LM (SIAIS074013; 6-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-6-oxohexanoic acid; CAS No.: 2172819-74-6). The compound (SIAIS630007) was obtained as white solid (13.4 mg, yield 34%). ¹H NMR (500 MHz, DMSO-d6) δ 9.04-8.98 (m, 2H), 8.79-8.74 (m, 1H), 8.57 (t, J=6.1 Hz, 1H), 8.53-8.42 (m, 1H), 8.31 (d, J=6.1 Hz, 1H), 7.86 (d, J=9.4 Hz, 2H), 7.53 (dd, J=15.9, 2.6 Hz, 1H), 7.44-7.35 (m, 4H), 7.27 (dd, J=11.5, 8.6 Hz, 2H), 7.02-6.94 (m, 2H), 6.88 (dd, J=15.9, 7.5 Hz, 1H), 4.54 (d, J=9.4 Hz, 1H), 4.47-4.39 (m, 2H), 4.38-4.32 (m, 1H), 4.21 (dd, J=15.9, 5.5 Hz, 1H), 3.65-3.57 (m, 10H), 3.43 (d, J=5.6 Hz, 2H), 3.25-3.14 (m, 6H), 2.44 (s, 3H), 2.37-2.33 (m, 1H), 2.31-2.25 (m, 1H), 2.20-2.11 (m, 1H), 2.06-2.00 (m, 1H), 1.93-1.88 (m, 1H), 1.69-1.61 (m, 2H), 1.53-1.42 m, 7H), 1.35-1.20 (m, 4H), 1.09-1.02 (m, 2H), 0.93 (s, 9H). HRMS (ESI) calcd for C₅₆H₇₄N₉O₇S⁺ [M+H]⁺, 1016.5426; found, 1016.5428.

### Example 32: Preparation of (2S,4R)-1-((S)-3,3-dimethyl-2-(7-oxo-7-(4-(4-(4-(4-((E)-3-(pyridin-3-yl)acrylamido)butyl)piperidine-1-carbonyl)phenyl)piperazin-1-yl)heptanamido)butanoyl)-4-hydroxy-N-(4-(4-methylthiazol-5-yl)benzyl)pyrrolidine-2-carboxamide (SIAIS631004)

Referring to the methods of example 1 and Scheme 15, the compound (SIAIS631004) was prepared by using Daporinad derivative 1 (SIAIS630006) and intermediate LM (SIAIS074014; 7-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-7-oxoheptanoic acid; CAS No.: 2162120-87-6). The compound (SIAIS631004) was obtained as white solid (12.1 mg, yield 30%). ¹H NMR (500 MHz, DMSO-d6) δ 9.08 (d, J=2.0 Hz, 1H), 9.06 (d, J=3.2 Hz, 1H), 8.83 (d, J=4.3 Hz, 1H), 8.64 (d, J=8.3 Hz, 1H), 8.58 (t, J=6.1 Hz, 1H), 8.40 (q, J=5.4 Hz, 1H), 8.01 (dd, J=8.2, 5.5 Hz, 1H), 7.85 (dd, J=9.4, 2.7 Hz, 1H), 7.56 (d, J=15.9 Hz, 1H), 7.44-7.34 (m, 4H), 7.27 (dd, J=8.8, 2.5 Hz, 2H), 7.05-6.91 (m, 3H), 4.53 (d, J=9.3 Hz, 1H), 4.45-4.40 (m, 3H), 4.36-4.32 (m, 1H), 4.21 (dd, J=15.9, 5.5 Hz, 1H), 3.71-3.56 (m, 6H), 3.45 (dd, J=6.5, 4.0 Hz, 1H), 3.25 (t, J=5.2 Hz, 1H), 3.23-3.15 (m, 5H), 2.95-2.65 (m, 2H), 2.44 (s, 3H), 2.33 (t, J=7.5 Hz, 1H), 2.26-2.21 (m, 1H), 2.19-2.08 (m, 1H), 2.06-1.99 (m, 1H), 1.94-1.90 (m, 1H), 1.72-1.62 (m, 2H), 1.54-1.40 (m, 7H), 1.35-1.20 (m, 7H), 1.08-1.00 (m, 2H), 0.93 (s, 9H). HRMS (ESI) calcd for C₅₇H₇₆N₉O₇S⁺ [M+H]⁺, 1030.5583; found, 1030.5585.

### Example 33: Preparation of (2S,4R)-1-((S)-3,3-dimethyl-2-(8-oxo-8-(4-(4-(4-(4-((E)-3-(pyridin-3-yl)acrylamido)butyl)piperidine-1-carbonyl)phenyl)piperazin-1-yl)octanamido)butanoyl)-4-hydroxy-N-(4-(4-methylthiazol-5-yl)benzyl)pyrrolidine-2-carboxamide (SIAIS631005)

Referring to the methods of example 1 and Scheme 15, the compound (SIAIS631005) was prepared by using Daporinad derivative 1 (SIAIS630006) and intermediate LM (SIAIS074015; 8-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-8-oxooctanoic acid; CAS No.: 2172819-75-7). The compound (SIAIS631005) was obtained as white solid (14.8 mg, yield 36%). ¹H NMR (500 MHz, CD₃OD) δ 9.17 (d, J=23.5 Hz, 1H), 9.03 (d, J=2.0 Hz, 1H), 8.84-8.70 (m, 2H), 8.14-7.96 (m, 1H), 7.63 (d, J=15.8 Hz, 1H), 7.52-7.47 (m, 2H), 7.46-7.43 (m, 2H), 7.38-7.30 (m, 2H), 7.05 (dd, J=26.6, 8.8 Hz, 2H), 6.92 (d, J=15.8 Hz, 1H), 4.64 (s, 1H), 4.61-4.53 (m, 2H), 4.50 (s, 1H), 4.42-4.33 (m, 1H), 3.91 (d, J=11.0 Hz, 1H), 3.80 (dd, J=10.9, 3.9 Hz, 1H), 3.75-3.69 (m, 3H), 3.53-3.47 (m, 1H), 3.40-3.33 (m, 12H), 3.25 (t, J=5.4 Hz, 1H), 2.50 (s, 3H), 2.45 (t, J=7.6 Hz, 1H), 2.34-2.26 (m, 2H), 2.25-2.19 (m, 1H), 2.10-2.05 (m, 1H), 1.67-1.55 (m, 8H), 1.47-1.29 (m, 9H), 1.20-1.12 (m, 2H), 1.03 (s, 9H). HRMS (ESI) calcd for C₅₈H₇₈N₉O₇S⁺ [M+H]⁺, 1044.5739; found, 1044.5741.

### Example 34: Preparation of (2S,4R)-1-((S)-3,3-dimethyl-2-(9-oxo-9-(4-(4-(4-(4-((E)-3-(pyridin-3-yl)acrylamido)butyl)piperidine-1-carbonyl)phenyl)piperazin-1-yl)nonanamido)butanoyl)-4-hydroxy-N-(4-(4-methylthiazol-5-yl)benzyl)pyrrolidine-2-carboxamide (SIAIS631006)

Referring to the methods of example 1 and Scheme 15, the compound (SIAIS631006) was prepared by using Daporinad derivative 1 (SIAIS630006) and intermediate LM (SIAIS074016; 9-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-9-oxononanoic acid; CAS No.: 2172819-76-8). The compound (SIAIS631006) was obtained as white solid (18 mg, yield 43%). ¹H NMR (500 MHz, CD₃OD) δ 8.93 (d, J=7.1 Hz, 1H), 8.90 (d, J=2.1 Hz, 1H), 8.68 (dd, J=5.3, 1.4 Hz, 1H), 8.47 (d, J=8.2 Hz, 1H), 7.82 (dd, J=8.1, 5.4 Hz, 1H), 7.60 (d, J=15.9 Hz, 1H), 7.47 (d, J=8.1 Hz, 2H), 7.42 (d, J=8.3 Hz, 2H), 7.32 (d, J=8.8 Hz, 2H), 7.00 (d, J=8.7 Hz, 2H), 6.84 (d, J=15.9 Hz, 1H), 4.63 (s, 1H), 4.58 (d, J=8.7 Hz, 1H), 4.54 (d, J=15.8 Hz, 1H), 4.50 (d, J=4.1 Hz, 1H), 4.36 (d, J=15.4 Hz, 1H), 3.91 (d, J=11.0 Hz, 1H), 3.80 (dd, J=11.0, 3.9 Hz, 1H), 3.74-3.68 (m, 4H), 3.40-3.33 (m, 12H), 3.23 (t, J=5.4 Hz, 1H), 2.48 (s, 3H), 2.44 (t, J=7.6 Hz, 1H), 2.32-2.26 (m, 2H), 2.26-2.19 (m, 1H), 2.11-2.05 (m, 1H), 1.65-1.56 (m, 8H), 1.41-1.31 (m, 11H), 1.22-1.11 (m, 2H), 1.03 (s, 9H). HRMS (ESI) calcd for C₅₉H₈₀N₉O₇S⁺ [M+H]⁺, 1058.5896; found, 1058.5898.

### Example 35: Preparation of (2S,4R)-1-((S)-3,3-dimethyl-2-(10-oxo-10-(4-(4-(4-(4-((E)-3-(pyridin-3-yl)acrylamido)butyl)piperidine-1-carbonyl)phenyl)piperazin-1-yl)decanamido)butanoyl)-4-hydroxy-N-(4-(4-methylthiazol-5-yl)benzyl)pyrrolidine-2-carboxamide (SIAIS631007)

Referring to the methods of example 1 and Scheme 15, the compound (SIAIS631007) was prepared by using Daporinad derivative 1 (SIAIS630006) and intermediate LM (SIAIS074019; 10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecanoic acid; CAS No.: 2172819-77-9). The compound (SIAIS631007) was obtained as white solid (18.3 mg, yield 43%). ¹H NMR (500 MHz, DMSO-d6) δ 9.11-9.00 (m, 2H), 8.84-8.73 (m, 1H), 8.64-8.51 (m, 2H), 8.36 (t, J=5.6 Hz, 1H), 7.96 (dd, J=8.2, 5.4 Hz, 1H), 7.83 (d, J=9.3 Hz, 1H), 7.55 (d, J=15.9 Hz, 1H), 7.45-7.36 (m, 4H), 7.26 (d, J=8.5 Hz, 2H), 6.99 (dd, J=8.9, 3.7 Hz, 2H), 6.92 (d, J=15.9 Hz, 1H), 4.54 (d, J=9.4 Hz, 1H), 4.46-4.38 (m, 3H), 4.35 (dd, J=4.8, 2.5 Hz, 1H), 4.21 (dd, J=15.9, 5.5 Hz, 1H), 3.68-3.62 (m, 2H), 3.59 (d, J=5.4 Hz, 4H), 3.44 (dd, J=6.6, 3.9 Hz, 1H), 3.26-3.14 (m, 6H), 2.95-2.70 (m, 2H), 2.44 (s, 3H), 2.33 (t, J=7.5 Hz, 2H), 2.28-2.20 (m, 1H), 2.13-2.05 (m, 1H), 2.07-2.00 (m, 1H), 1.95-1.86 (m, 1H), 1.69-1.62 (m, 2H), 1.52-1.42 (m, 7H), 1.36-1.20 (m, 13H), 1.10-0.98 (m, 2H), 0.93 (s, 9H). HRMS (ESI) calcd for C₆₀H₈₂N₉O₇S⁺ [M+H]⁺, 1072.6052; found, 1072.6055.

### Example 36: Preparation of (2S,4R)-1-((S)-3,3-dimethyl-2-(11-oxo-11-(4-(4-(4-(4-((E)-3-(pyridin-3-yl)acrylamido)butyl)piperidine-1-carbonyl)phenyl)piperazin-1-yl)undecanamido)butanoyl)-4-hydroxy-N-(4-(4-methylthiazol-5-yl)benzyl)pyrrolidine-2-carboxamide (SIAIS631008)

Referring to the methods of example 1 and Scheme 15, the compound (SIAIS631008) was prepared by using Daporinad derivative 1 (SIAIS630006) and intermediate LM (SIAIS074020; 11-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-11-oxoundecanoic acid; CAS No.: 2172819-78-0). The compound (SIAIS631008) was obtained as white solid (21 mg, yield 49%). ¹H NMR (500 MHz, DMSO-d6) δ 9.10-9.01 (m, 2H), 8.83 (dd, J=5.5, 1.3 Hz, 1H), 8.65-8.51 (m, 2H), 8.38 (t, J=5.6 Hz, 1H), 8.00 (dd, J=8.2, 5.5 Hz, 1H), 7.84 (d, J=9.3 Hz, 1H), 7.56 (d, J=15.9 Hz, 1H), 7.45-7.37 (m, 4H), 7.28 (d, J=8.6 Hz, 2H), 7.01 (d, J=8.8 Hz, 2H), 6.94 (d, J=15.9 Hz, 1H), 4.54 (d, J=9.4 Hz, 1H), 4.48-4.40 (m, 3H), 4.38-4.33 (m, 1H), 4.22 (dd, J=15.9, 5.6 Hz, 1H), 3.68-3.59 (m, 6H), 3.34-3.12 (m, 7H), 2.95-2.70 (m, 2H), 2.45 (s, 3H), 2.34 (t, J=7.5 Hz, 2H), 2.28-2.23 (m, 1H), 2.13-2.08 (m, 1H), 2.07-2.00 (m, 1H), 1.93-1.88 (m, 1H), 1.67 (d, J=12.8 Hz, 2H), 1.55-1.43 (m, 8H), 1.36-1.30 (m, 2H), 1.28-1.20 (m, 12H), 1.08-0.99 (m, 2H), 0.93 (s, 9H). HRMS (ESI) calcd for C₆₁H₈₄N₉O₇S⁺ [M+H]⁺, 1086.6209; found, 1086.6211.

### Example 37: Preparation of (2S,4R)-1-((S)-3,3-dimethyl-2-(2-(2-(2-oxo-2-(4-(4-(4-(4-((E)-3-(pyridin-3-yl)acrylamido)butyl)piperidine-1-carbonyl)phenyl)piperazin-1-yl)ethoxy)ethoxy)acetamido)butanoyl)-4-hydroxy-N-(4-(4-methylthiazol-5-yl)benzyl)pyrrolidine-2-carboxamide (SIAIS631010)

Referring to the methods of example 1 and Scheme 15, the compound (SIAIS631010) was prepared by using Daporinad derivative 1 (SIAIS630006) and intermediate LM (SIAIS151010; 2-(2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)acetic acid; CAS No.: 2172820-08-3). The compound (SIAIS631010) was obtained as white solid (14.1 mg, yield 34%). ¹H NMR (500 MHz, CD₃OD) δ 9.18 (d, J=38.0 Hz, 1H), 9.03 (d, J=2.0 Hz, 1H), 8.79 (d, J=5.5 Hz, 1H), 8.81-8.74 (m, 1H), 8.09-8.04 (m, 1H), 7.63 (d, J=15.9 Hz, 1H), 7.53-7.39 (m, 4H), 7.36 (d, J=8.8 Hz, 1H), 7.31 (d, J=8.8 Hz, 1H), 7.08 (d, J=8.9 Hz, 1H), 6.99 (d, J=8.9 Hz, 1H), 6.93 (dd, J=15.9, 1.6 Hz, 1H), 4.70 (d, J=8.3 Hz, 1H), 4.61-4.54 (m, 2H), 4.50 (t, J=7.7 Hz, 1H), 4.37 (s, 1H), 4.06 (dd, J=6.8, 2.2 Hz, 2H), 3.88 (d, J=10.8 Hz, 1H), 3.81 (dd, J=11.1, 3.7 Hz, 1H), 3.77-3.73 (m, 4H), 3.73-3.69 (m, 1H), 3.53-3.49 (m, 2H), 3.38 (dd, J=6.6, 3.9 Hz, 2H), 3.35-3.32 (m, 11H), 3.28-3.22 (m, 1H), 2.52 (s, 3H), 2.24 (dd, J=13.2, 7.7 Hz, 1H), 2.13-2.07 (m, 1H), 1.78-1.70 (m, 1H), 1.62-1.55 (m, 3H), 1.47-1.40 (m, 2H), 1.38-1.30 (m, 2H), 1.22-1.10 (m, 2H), 1.04 (s, 9H). HRMS (ESI) calcd for C₅₆H₇₄N₉O₉S⁺ [M+H]⁺, 1048.5325; found, 1048.5327.

### Example 38: Preparation of (2S,4R)-1-((S)-3,3-dimethyl-2-(3-(2-(3-oxo-3-(4-(4-(4-(4-((E)-3-(pyridin-3-yl)acrylamido)butyl)piperidine-1-carbonyl)phenyl)piperazin-1-yl)propoxy)ethoxy)propanamido)butanoyl)-4-hydroxy-N-(4-(4-methylthiazol-5-yl)benzyl)pyrrolidine-2-carboxamide (SIAIS631011)

Referring to the methods of example 1 and Scheme 15, the compound (SIAIS631011) was prepared by using Daporinad derivative 1 (SIAIS630006) and intermediate LM (SIAIS151002; 3-(2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethoxy)propanoic acid; CAS No.: 2172820-09-4). The compound (SIAIS631011) was obtained as white solid (14.2 mg, yield 33%). ¹H NMR (500 MHz, CD₃OD) δ 9.45 (d, J=34.6 Hz, 1H), 9.06 (d, J=1.9 Hz, 1H), 8.86-8.80 (m, 2H), 8.11 (dd, J=8.2, 5.8 Hz, 1H), 7.64 (d, J=15.8 Hz, 1H), 7.55-7.44 (m, 4H), 7.35 (t, J=9.2 Hz, 2H), 7.07 (dd, J=11.4, 8.8 Hz, 2H), 6.95 (dd, J=15.8, 1.8 Hz, 1H), 4.65 (s, 1H), 4.60-4.53 (m, 2H), 4.52-4.47 (m, 1H), 4.37 (d, J=15.7 Hz, 1H), 3.89 (d, J=10.9 Hz, 1H), 3.84-3.67 (m, 9H), 3.63-3.57 (m, 4H), 3.51 (dd, J=6.6, 3.9 Hz, 1H), 3.42-3.33 (m, 11H), 3.27 (d, J=5.3 Hz, 1H), 2.71 (t, J=6.3 Hz, 1H), 2.53 (s, 3H), 2.48-2.42 (m, 1H), 2.26-2.20 (m, 1H), 2.12-2.04 (m, 1H), 1.85-1.70 (m, 1H), 1.59 (p, J=7.2 Hz, 3H), 1.47-1.40 (m, 2H), 1.38-1.30 (m, 2H), 1.22-1.11 (m, 2H), 1.03 (s, 9H). HRMS (ESI) calcd for C₅₈H₇₈N₉O₉S⁺ [M+H]⁺, 1076.5638; found, 1076.5641.

### Example 39: Preparation of (2S,4R)-1-((S)-2-(tert-butyl)-4,16-dioxo-16-(4-(4-(4-(4-((E)-3-(pyridin-3-yl)acrylamido)butyl)piperidine-1-carbonyl)phenyl)piperazin-1-yl)-7,10,13-trioxa-3-azahexadecanoyl)-4-hydroxy-N-(4-(4-methylthiazol-5-yl)benzyl)pyrrolidine-2-carboxamide (SIAIS631012)

Referring to the methods of example 1 and Scheme 15, the compound (SIAIS631012) was prepared by using Daporinad derivative 1 (SIAIS630006) and intermediate LM (SIAIS151003; (S)-15-((2S,4R)-4-hydroxy-2-((4-(4-methylthiazol-5-yl)benzyl)carbamoyl)pyrrolidine-1-carbonyl)-16,16-dimethyl-13-oxo-4,7,10-trioxa-14-azaheptadecanoic acid; CAS No.: 2140807-42-5). The compound (SIAIS631012) was obtained as white solid (14.8 mg, yield 34%). ¹H NMR (500 MHz, CD₃OD) δ 9.52 (d, J=11.8 Hz, 1H), 9.00 (d, J=2.0 Hz, 1H), 8.74 (dd, J=12.9, 7.0 Hz, 2H), 8.03 (dd, J=8.2, 5.7 Hz, 1H), 7.54 (d, J=15.8 Hz, 1H), 7.46-7.34 (m, 4H), 7.31-7.23 (m, 2H), 7.07 (d, J=8.7 Hz, 2H), 7.00-6.95 (m, 1H), 6.92 (d, J=15.9 Hz, 1H), 4.55 (d, J=5.1 Hz, 1H), 4.50-4.46 (m, 1H), 4.45-4.39 (m, 2H), 4.28 (d, J=15.8 Hz, 1H), 3.81 (d, J=11.1 Hz, 1H), 3.75-3.59 (m, 8H), 3.57-3.48 (m, 10H), 3.44-3.39 (m, 2H), 3.34-3.23 (m, 7H), 2.67 (t, J=6.1 Hz, 1H), 2.53-2.36 (m, 6H), 2.17-2.12 (m, 1H), 2.01-1.95 (m, 1H), 1.67 (d, J=43.4 Hz, 2H), 1.52-1.46 (m, 3H), 1.34-1.30 (m, 2H), 1.27-1.20 (m, 2H), 1.04 (br, 2H), 0.94 (s, 9H). HRMS (ESI) calcd for C₆₀H₈₂N₉O₁₀S⁺ [M+H]⁺, 1120.5900; found, 1120.5905.

### Example 40: Preparation of (2S,4R)-1-((S)-2-(tert-butyl)-4,19-dioxo-19-(4-(4-(4-(4-((E)-3-(pyridin-3-yl)acrylamido)butyl)piperidine-1-carbonyl)phenyl)piperazin-1-yl)-7,10,13,16-tetraoxa-3-azanonadecanoyl)-4-hydroxy-N-(4-(4-methylthiazol-5-yl)benzyl)pyrrolidine-2-carboxamide (SIAIS631013)

Referring to the methods of example 1 and Scheme 15, the compound (SIAIS631013) was prepared by using Daporinad derivative 1 (SIAIS630006) and intermediate LM (SIAIS151008; (S)-18-((2S,4R)-4-hydroxy-2-((4-(4-methylthiazol-5-yl)benzyl)carbamoyl)pyrrolidine-1-carbonyl)-19,19-dimethyl-16-oxo-4,7,10,13-tetraoxa-17-azaeicosanoic acid; CAS No.: 2172820-12-9). The compound (SIAIS631013) was obtained as white solid (15.2 mg, yield 33%). ¹H NMR (500 MHz, CD₃OD) δ 9.59 (d, J=1.6 Hz, 1H), 9.09 (d, J=2.1 Hz, 1H), 8.86-8.82 (m, 2H), 8.17-8.09 (m, 1H), 7.64 (d, J=16.0 Hz, 1H), 7.56-7.47 (m, 4H), 7.39-7.29 (m, 2H), 7.08 (d, J=8.9 Hz, 2H), 7.00 (d, J=15.8 Hz, 1H), 4.65 (s, 1H), 4.61-4.54 (m, 2H), 4.52-4.50 (m, 1H), 4.38 (d, J=15.7 Hz, 1H), 3.90 (d, J=12.1 Hz, 1H), 3.84-3.78 (m, 2H), 3.77-3.70 (m, 4H), 3.67-3.59 (m, 12H), 3.55-3.49 (m, 3H), 3.40-3.36 (m, 4H), 3.36-3.28 (m, 6H), 2.64-2.46 (m, 7H), 2.28-2.20 (m, 1H), 2.11-2.05 (m, 1H), 1.81 (br, 2H), 1.60 (p, J=6.9 Hz, 3H), 1.46-1.40 (m, 2H), 1.36-1.32 (m, 2H), 1.20-1.10 (m, 2H), 1.04 (s, 9H). HRMS (ESI) calcd for C₆₂H₈₆N₉O₁₁S⁺ [M+H]⁺, 1164.6162; found, 1164.6165.

### Example 41: Preparation of (2S,4R)-1-((S)-2-(tert-butyl)-4,22-dioxo-22-(4-(4-(4-(4-((E)-3-(pyridin-3-yl)acrylamido)butyl)piperidine-1-carbonyl)phenyl)piperazin-1-yl)-7,10,13,16,19-pentaoxa-3-azadocosanoyl)-4-hydroxy-N-(4-(4-methylthiazol-5-yl)benzyl)pyrrolidine-2-carboxamide (SIAIS631014)

Referring to the methods of example 1 and Scheme 15, the compound (SIAIS631014) was prepared by using Daporinad derivative 1 (SIAIS630006) and intermediate LM (SIAIS151009; (S)-21-((2S,4R)-4-hydroxy-2-((4-(4-methylthiazol-5-yl)benzyl)carbamoyl)pyrrolidine-1-carbonyl)-22,22-dimethyl-19-oxo-4,7,10,13,16-pentaoxa-20-azadocosanoic acid; CAS No.: 2172820-14-1). The compound (SIAIS631014) was obtained as white solid (17.6 mg, yield 36%). ¹H NMR (500 MHz, CD₃OD) δ 9.10 (d, J=12.4 Hz, 1H), 8.96 (s, 1H), 8.75-8.61 (m, 2H), 7.98 (dd, J=8.2, 5.7 Hz, 1H), 7.53 (d, J=15.8 Hz, 1H), 7.42-7.30 (m, 4H), 7.25 (dd, J=10.0, 8.7 Hz, 2H), 6.96 (dd, J=15.0, 8.8 Hz, 2H), 6.87 (dd, J=15.9, 2.0 Hz, 1H), 4.55 (s, 1H), 4.50-4.46 (m, 1H), 4.45-4.39 (m, 2H), 4.27 (d, J=15.6 Hz, 1H), 3.83-3.75 (m, 1H), 3.73-3.60 (m, 10H), 3.55-3.50 (m, 18H), 3.42-3.41 (m, 1H), 3.31-3.22 (m, 5H), 3.18 (t, J=5.3 Hz, 1H), 2.63 (t, J=6.1 Hz, 1H), 2.53-2.43 (m, 2H), 2.41-2.37 (m, 4H), 2.16-2.11 (m, 1H), 2.05-1.95 (m, 1H), 1.66 (br, 2H), 1.52-1.46 (m, 3H), 1.36-1.28 (m, 2H), 1.24-1.22 (m, 2H), 1.10-1.02 (m, 2H), 0.94 (s, 9H). HRMS (ESI) calcd for C₆₄H₉₀N₉O₁₂S⁺ [M+H]⁺, 1208.6424; found, 1208.6427.

### Example 42: Preparation of (2S,4R)-1-((S)-3,3-dimethyl-2-(16-oxo-16-(4-(4-(4-(4-((E)-3-(pyridin-3-yl)acrylamido)butyl)piperidine-1-carbonyl)phenyl)piperazin-1-yl)hexadecanamido)butanoyl)-4-hydroxy-N-(4-(4-methylthiazol-5-yl)benzyl)pyrrolidine-2-carboxamide (SIAIS630008)

Referring to the methods of example 1 and Scheme 15, the compound (SIAIS630008) was prepared by using Daporinad derivative 1 (SIAIS630006) and intermediate LM (SIAIS164189). The compound (SIAIS630008) was obtained as white solid (7.0 mg, yield 37%). ¹H NMR (500 MHz, CD₃OD) δ 8.87 (s, 1H), 8.72 (s, 1H), 8.65 (s, 1H), 8.52 (d, J=5.0 Hz, 1H), 8.07 (d, J=8.2 Hz, 1H), 7.80 (d, J=8.7 Hz, 1H), 7.55 (d, J=15.8 Hz, 1H), 7.46 (d, J=7.8 Hz, 3H), 7.42-7.39 (m, 2H), 7.32 (d, J=8.2 Hz, 1H), 7.00 (d, J=8.3 Hz, 1H), 6.73 (d, J=15.9 Hz, 1H), 4.64 (d, J=6.1 Hz, 1H), 4.58-4.47 (m, 3H), 4.35 (d, J=15.5 Hz, 1H), 3.90 (d, J=11.2 Hz, 1H), 3.81-3.78 (m, 1H), 3.73-3.68 (m, 3H), 3.63 (d, J=10.5 Hz, 1H), 3.55 (d, J=17.9 Hz, 2H), 3.45-3.44 (m, 1H), 3.35-3.32 (m, 6H), 3.17-3.16 (m, 1H), 2.47 (s, 3H), 2.31-2.17 (m, 4H), 2.10-2.02 (m, 2H), 1.59 (br, 10H), 1.28 (br, 25H), 1.03 (s, 9H). HRMS (ESI) calcd for C₆₆H₉₄N₉O₇S⁺ [M+H]⁺, 1156.6991; found, 1156.6995.

### Example 43: Preparation of (E)-N-(4-(1-(4-(4-(4-((2-(2,6-dioxopiperidin-3-yl)-1-oxoisoindolin-4-yl)oxy)butyl)piperazin-1-yl)benzoyl)piperidin-4-yl)butyl)-3-(pyridin-3-yl)acrylamide (SIAIS631020)

Referring to the methods of example 15 and Scheme 16, the compound (SIAIS631020) was prepared by using Daporinad derivative 1 (SIAIS630006) and intermediate LM (SIAIS1222171; 3-(4-(4-bromobutoxy)-1-oxoisoindolin-2-yl)piperidine-2,6-dione; CAS No.: 2641288-70-0). The compound (SIAIS631020) was obtained as white solid (6.8 mg, yield 22%). ¹H NMR (500 MHz, CD₃OD) δ 8.90 (d, J=2.2 Hz, 1H), 8.67 (dd, J=5.4, 1.5 Hz, 1H), 8.46 (d, J=8.2 Hz, 1H), 7.81 (dd, J=8.1, 5.3 Hz, 1H), 7.59 (d, J=15.8 Hz, 1H), 7.50 (t, J=7.8 Hz, 1H), 7.40 (d, J=7.3 Hz, 1H), 7.35 (d, J=8.8 Hz, 2H), 7.23 (d, J=8.1 Hz, 1H), 7.07 (d, J=8.9 Hz, 2H), 6.86 (d, J=15.8 Hz, 1H), 5.16 (dd, J=13.3, 5.2 Hz, 1H), 4.51 (d, J=17.3 Hz, 1H), 4.43 (d, J=17.3 Hz, 1H), 4.23 (t, J=5.8 Hz, 2H), 4.01-3.79 (m, 3H), 3.70 (br, 2H), 3.35-3.32 (m, 4H), 3.29-3.21 (m, 6H), 2.96-2.86 (m, 1H), 2.81-2.76 (m, 1H), 2.56-2.47 (m, 1H), 2.24-2.15 (m, 1H), 2.10-2.01 (m, 2H), 2.01-1.92 (m, 2H), 1.84-1.69 (m, 2H), 1.59 (p, J=7.1 Hz, 3H), 1.47-1.39 (m, 2H), 1.38-1.27 (m, 2H), 1.16 (br, 2H). HRMS (ESI) calcd for C₄₅H₅₆N₇O₆⁺ [M+H]⁺, 790.4287; found, 790.4289.

### Example 44: Preparation of (E)-N-(4-(1-(4-(4-(8-((2-(2,6-dioxopiperidin-3-yl)-1-oxoisoindolin-4-yl)oxy)octyl)piperazin-1-yl)benzoyl)piperidin-4-yl)butyl)-3-(pyridin-3-yl)acrylamide (SIAIS631024)

Referring to the methods of example 15 and Scheme 16, the compound (SIAIS631024) was prepared by using Daporinad derivative 1 (SIAIS630006) and intermediate LM (SIAIS1222063). The compound (SIAIS631024) was obtained as white solid (5.4 mg, 16%). ¹H NMR (500 MHz, DMSO-d6) δ 11.10 (s, 1H), 10.97 (s, 1H), 9.03 (s, 1H), 8.80 (d, J=5.4 Hz, 1H), 8.55 (d, J=8.1 Hz, 1H), 8.37 (t, J=5.6 Hz, 1H), 7.94 (t, J=6.9 Hz, 1H), 7.54 (d, J=15.9 Hz, 1H), 7.47 (t, J=7.8 Hz, 1H), 7.29 (dd, J=10.4, 8.0 Hz, 3H), 7.24 (d, J=8.2 Hz, 1H), 7.01 (d, J=8.6 Hz, 2H), 6.92 (d, J=15.9 Hz, 1H), 5.11 (dd, J=13.3, 5.1 Hz, 1H), 4.37 (d, J=17.3 Hz, 1H), 4.22 (d, J=17.4 Hz, 1H), 4.12 (t, J=6.4 Hz, 2H), 3.88 (d, J=13.0 Hz, 2H), 3.53 (d, J=11.8 Hz, 2H), 3.29-3.15 (m, 5H), 3.11-3.01 (m, 5H), 2.95-2.86 (m, 2H), 2.64-2.52 (m, 1H), 2.48-2.42 (m, 1H), 2.05-1.95 (m, 1H), 1.79-1.70 (m, 4H), 1.66 (br, 2H), 1.46 (p, J=6.9 Hz, 5H), 1.39-1.29 (m, 9H), 1.27-1.19 (m, 2H), 1.10-1.00 (m, 2H). HRMS (ESI) calcd for C₄₉H₆₄N₇O₆⁺ [M+H]⁺, 846.4913; found, 846.4915.

### Example 45: Preparation of (E)-N-(4-(4-(4-(4-(7-(2-(2,6-dioxopiperidin-3-yl)-1-oxoisoindolin-4-yl)hept-6-yn-1-yl)piperazin-1-yl)benzoyl)piperazin-1-yl)butyl)-3-(pyridin-3-yl)acrylamide (SIAIS631045)

Referring to the methods of example 15 and Scheme 16, the compound (SIAIS631045) was prepared by using Daporinad derivative 2 (SIAIS630020) and intermediate LM (SIAIS292017; 7-(2-(2,6-dioxopiperidin-3-yl)-1-oxoisoindolin-4-yl)hept-6-yn-1-yl methanesulfonate; CAS No.: 2570254-41-8). The compound (SIAIS631045) was obtained as white solid (12.4 mg, yield 39%). ¹H NMR (500 MHz, CD₃OD) δ 9.00 (s, 1H), 8.75 (s, 1H), 8.65 (s, 1H), 7.96 (s, 1H), 7.75 (dd, J=7.6, 1.1 Hz, 1H), 7.69-7.58 (m, 2H), 7.51 (t, J=7.7 Hz, 1H), 7.47 (d, J=8.8 Hz, 2H), 7.10 (d, J=8.9 Hz, 2H), 6.95 (d, J=14.5 Hz, 1H), 5.19 (dd, J=13.3, 5.2 Hz, 1H), 4.54 (d, J=17.4 Hz, 1H), 4.48 (d, J=17.5 Hz, 1H), 3.99 (d, J=12.8 Hz, 2H), 3.69 (d, J=11.4 Hz, 2H), 3.63 (br, 1H), 3.40 (t, J=6.9 Hz, 3H), 3.27-3.23 (m, 6H), 3.21-3.15 (m, 4H), 2.97-2.88 (m, 1H), 2.83-2.75 (m, 2H), 2.57 (t, J=6.9 Hz, 2H), 2.53 (dd, J=13.3, 4.6 Hz, 1H), 2.22-2.17 (m, 1H), 1.93-1.84 (m, 5H), 1.75-1.60 (m, 8H), 1.29 (br, 1H). HRMS (ESI) calcd for C₄₇H₅₇N₈O₅⁺ [M+H]⁺, 813.4446; found, 813.4449.

### Example 46: Preparation of (E)-N-(4-(1-(4-(4-(2-((2-(2,6-dioxopiperidin-3-yl)-1,3-dioxoisoindolin-4-yl)thio)ethyl)piperazin-1-yl)benzoyl)piperidin-4-yl)butyl)-3-(pyridin-3-yl)acrylamide (SIAIS631068)

Referring to the methods of example 15 and Scheme 16, the compound (SIAIS631068) was prepared by using Daporinad derivative 1 (SIAIS630006) and intermediate LM (4-((2-bromoethyl)thio)-2-(2,6-dioxopiperidin-3-yl)isoindoline-1,3-dione; CAS No.: 2378582-68-2). The compound (SIAIS631068) was obtained as yellow solid (11.6 mg, yield 37%). ¹H NMR (500 MHz, CD₃OD) δ 8.75 (s, 1H), 8.56 (s, 1H), 8.14 (s, 1H), 7.84 (d, J=5.9 Hz, 2H), 7.78 (dd, J=5.7, 2.4 Hz, 1H), 7.56 (d, J=14.0 Hz, 1H), 7.36 (d, J=8.7 Hz, 2H), 7.08 (d, J=8.6 Hz, 2H), 6.75 (d, J=16.6 Hz, 1H), 5.16 (dd, J=12.8, 5.4 Hz, 1H), 3.63-3.50 (m, 12H), 3.35 (br, 4H), 2.85 (br, 2H), 2.87-2.85 (m, 1H), 2.25-2.04 (m, 1H), 1.89 (br, 1H), 1.68-1.55 (m, 4H), 1.42 (br, 2H), 1.39-1.27 (m, 3H), 1.22-1.11 (m, 2H). HRMS (ESI) calcd for C₄₃H₅₀N₇O₆S⁺ [M+H]⁺, 792.3538; found, 792.3539.

### Example 47: Preparation of (E)-N-(4-(1-(4-(4-(3-((2-(2,6-dioxopiperidin-3-yl)-1,3-dioxoisoindolin-4-yl)thio)propyl)piperazin-1-yl)benzoyl)piperidin-4-yl)butyl)-3-(pyridin-3-yl)acrylamide (SIAIS631069)

Referring to the methods of example 15 and Scheme 16, the compound (SIAIS631069) was prepared by using Daporinad derivative 1 (SIAIS630006) and intermediate LM (4-((3-bromopropyl)thio)-2-(2,6-dioxopiperidin-3-yl)isoindoline-1,3-dione; CAS No.: 2378582-69-3). The compound (SIAIS631069) was obtained as yellow solid (6.9 mg, yield 22%). ¹H NMR (500 MHz, CD₃OD) δ 8.86 (s, 1H), 8.65 (d, J=5.0 Hz, 1H), 8.39 (d, J=8.5 Hz, 1H), 7.83-7.77 (m, 3H), 7.72-7.66 (m, 1H), 7.59 (d, J=15.8 Hz, 1H), 7.35 (d, J=8.8 Hz, 2H), 7.07 (d, J=8.9 Hz, 2H), 6.82 (d, J=15.9 Hz, 1H), 5.14 (dd, J=12.8, 5.5 Hz, 1H), 3.96 (br, 2H), 3.71-3.61 (m, 2H), 3.47-3.40 (m, 4H), 3.30-2.28 (m, 8H), 2.92-2.85 (m, 1H), 2.81-2.68 (m, 1H), 2.25-2.20 (m, 2H), 2.19-2.10 (m, 1H), 1.82 (br, 1H), 1.62-1.57 (m, 4H), 1.47-1.38 (m, 2H), 1.36-1.29 (m, 4H), 1.18 (br, 2H). HRMS (ESI) calcd for C₄₄H₅₂N₇O₆S⁺ [M+H]⁺, 806.3694; found, 806.3699.

### Example 48: Preparation of (E)-N-(4-(1-(4-(4-(4-((2-(2,6-dioxopiperidin-3-yl)-1,3-dioxoisoindolin-4-yl)thio)butyl)piperazin-1-yl)benzoyl)piperidin-4-yl)butyl)-3-(pyridin-3-yl)acrylamide (SIAIS631070)

Referring to the methods of example 15 and Scheme 16, the compound (SIAIS631070) was prepared by using Daporinad derivative 1 (SIAIS630006) and intermediate LM (4-((4-bromobutyl)thio)-2-(2,6-dioxopiperidin-3-yl)isoindoline-1,3-dione; CAS No.: 2378582-70-6). The compound (SIAIS631070) was obtained as yellow solid (7.1 mg, yield 22%). ¹H NMR (500 MHz, CD₃OD) δ 8.75 (s, 1H), 8.54 (s, 1H), 8.26 (s, 1H), 7.66 (d, J=4.6 Hz, 3H), 7.60-7.53 (m, 1H), 7.48 (d, J=15.8 Hz, 1H), 7.26 (d, J=8.8 Hz, 2H), 6.97 (d, J=8.9 Hz, 2H), 6.71 (d, J=15.8 Hz, 1H), 5.03 (dd, J=12.8, 5.5 Hz, 1H), 3.87 (br, 2H), 3.58 (br, 2H), 3.18-3.14 (m, 6H), 3.04 (br, 3H), 2.79-2.74 (m, 1H), 2.66-2.60 (m, 1H), 2.07-1.98 (m, 1H), 1.96-1.83 (m, 2H), 1.80-1.74 (m, 5H), 1.49 (p, J=7.2 Hz, 4H), 1.33-1.30 (m, 3H), 1.28-1.17 (m, 4H), 1.07 (br, 3H). HRMS (ESI) calcd for C₄₅H₅₄N₇O₆S⁺ [M+H]⁺, 820.3851; found, 820.3856.

### Example 49: Preparation of (E)-N-(4-(1-(4-(4-(5-((2-(2,6-dioxopiperidin-3-yl)-1,3-dioxoisoindolin-4-yl)thio)pentyl)piperazin-1-yl)benzoyl)piperidin-4-yl)butyl)-3-(pyridin-3-yl)acrylamide (SIAIS631071)

Referring to the methods of example 15 and Scheme 16, the compound (SIAIS631071) was prepared by using Daporinad derivative 1 (SIAIS630006) and intermediate LM (4-((5-bromopentyl)thio)-2-(2,6-dioxopiperidin-3-yl)isoindoline-1,3-dione; CAS No.: 2378582-71-7). The compound (SIAIS631071) was obtained as yellow solid (5.9 mg, yield 18%). ¹H NMR (500 MHz, CD₃OD) δ 8.98 (s, 1H), 8.75 (s, 1H), 8.65 (s, 1H), 7.97 (s, 1H), 7.79-7.69 (m, 2H), 7.70-7.55 (m, 2H), 7.45-7.22 (m, 2H), 7.07 (d, J=8.8 Hz, 2H), 6.89 (d, J=16.0 Hz, 1H), 5.12 (dd, J=12.7, 5.5 Hz, 1H), 3.97 (d, J=13.4 Hz, 2H), 3.68 (d, J=12.1 Hz, 2H), 3.34 (dd, J=8.1, 1.6 Hz, 6H), 3.27-3.06 (m, 9H), 2.88-2.84 (m, 1H), 2.78-2.66 (m, 1H), 2.19-2.06 (m, 1H), 1.86 (p, J=7.8 Hz, 4H), 1.67-1.56 (m, 4H), 1.43 (br, 2H), 1.38-1.26 (m, 4H), 1.16 (br, 3H). HRMS (ESI) calcd for C₄₆H₅₆N₇O₆S⁺ [M+H]⁺, 834.4007; found, 834.4009.

### Example 50: Preparation of (E)-N-(4-(1-(4-(4-(6-((2-(2,6-dioxopiperidin-3-yl)-1,3-dioxoisoindolin-4-yl)thio)hexyl)piperazin-1-yl)benzoyl)piperidin-4-yl)butyl)-3-(pyridin-3-yl)acrylamide (SIAIS631072)

Referring to the methods of example 15 and Scheme 16, the compound (SIAIS631072) was prepared by using Daporinad derivative 1 (SIAIS630006) and intermediate LM (4-((6-bromohexyl)thio)-2-(2,6-dioxopiperidin-3-yl)isoindoline-1,3-dione; CAS No.: 2378586-19-5). The compound (SIAIS631072) was obtained as yellow solid (6.9 mg, yield 21%). ¹H NMR (500 MHz, CD₃OD) δ 8.81 (d, J=2.2 Hz, 1H), 8.60 (dd, J=5.2, 1.5 Hz, 1H), 8.28 (dt, J=8.0, 1.9 Hz, 1H), 7.76-7.70 (m, 3H), 7.64-7.53 (m, 2H), 7.36 (d, J=8.8 Hz, 2H), 7.07 (d, J=8.9 Hz, 2H), 6.80 (d, J=15.8 Hz, 1H), 5.12 (dd, J=12.7, 5.5 Hz, 1H), 3.98-3.82 (m, 3H), 3.67 (br, 2H), 3.33 (d, J=7.3 Hz, 2H), 3.24-3.15 (m, 12H), 2.91-2.81 (m, 1H), 2.81-2.71 (m, 1H), 2.19-2.08 (m, 1H), 1.86-1.79 (m 5H), 1.66-1.58 (m, 4H), 1.52-1.48 (m, 4H), 1.44-1.40 (m, 2H), 1.36-1.32 (m, 2H), 1.16 (br, 2H). HRMS (ESI) calcd for C₄₇H₅₈N₇O₆S⁺ [M+H]⁺, 848.4164; found, 848.4168.

### Example 51: Preparation of (E)-N-(4-(1-(4-(4-(7-((2-(2,6-dioxopiperidin-3-yl)-1,3-dioxoisoindolin-4-yl)thio)heptyl)piperazin-1-yl)benzoyl)piperidin-4-yl)butyl)-3-(pyridin-3-yl)acrylamide (SIAIS631078)

Referring to the methods of example 15 and Scheme 16, the compound (SIAIS631078) was prepared by using Daporinad derivative 1 (SIAIS630006) and intermediate LM (4-((7-bromoheptyl)thio)-2-(2,6-dioxopiperidin-3-yl)isoindoline-1,3-dione; CAS No.: 2378586-22-0). The compound (SIAIS631078) was obtained as yellow solid (7.1 mg, yield 21%). ¹H NMR (500 MHz, DMSO-d6) δ 11.12 (s, 1H), 10.69 (s, 1H), 8.87 (s, 1H), 8.66 (d, J=5.0 Hz, 1H), 8.30-8.17 (m, 2H), 7.83-7.71 (m, 2H), 7.70-7.61 (m, 2H), 7.48 (d, J=15.9 Hz, 1H), 7.28 (d, J=8.6 Hz, 2H), 7.01 (d, J=8.9 Hz, 2H), 6.81 (d, J=16.0 Hz, 1H), 5.11 (dd, J=12.9, 5.4 Hz, 1H), 3.89 (d, J=12.9 Hz, 2H), 3.54 (d, J=12.0 Hz, 2H), 3.25-3.03 (m, 10H), 2.94-2.85 (m, 1H), 2.64-2.56 (m 1H), 2.55-2.52 (m, 1H), 2.10-2.02 (m, 1H), 1.79-1.61 (m, 6H), 1.46 (h, J=7.5 Hz, 5H), 1.39-1.20 (m, 9H), 1.12-0.98 (m, 2H). HRMS (ESI) calcd for C₄₈H₆₀N₇O₆S⁺ [M+H]⁺, 862.4320; found, 862.4324.

### Example 52: Preparation of (E)-N-(4-(1-(4-(4-(8-((2-(2,6-dioxopiperidin-3-yl)-1,3-dioxoisoindolin-4-yl)thio)octyl)piperazin-1-yl)benzoyl)piperidin-4-yl)butyl)-3-(pyridin-3-yl)acrylamide (SIAIS631073)

Referring to the methods of example 15 and Scheme 16, the compound (SIAIS631073) was prepared by using Daporinad derivative 1 (SIAIS630006) and intermediate LM (SIAIS1224003). The compound (SIAIS631073) was obtained as yellow solid (7.7 mg, yield 22%). ¹H NMR (500 MHz, CD₃OD) δ 8.81 (d, J=2.3 Hz, 1H), 8.60 (dd, J=5.1, 1.5 Hz, 1H), 8.27 (dt, J=8.0, 1.9 Hz, 1H), 7.77-7.68 (m, 3H), 7.63-7.54 (m, 2H), 7.36 (d, J=8.8 Hz, 2H), 7.07 (d, J=8.9 Hz, 2H), 6.80 (d, J=15.9 Hz, 1H), 5.12 (dd, J=12.7, 5.5 Hz, 1H), 3.96-3.82 (m, 3H), 3.67 (br, 2H), 3.33 (d, J=7.4 Hz, 2H), 3.24-3.10 (m, 11H), 2.89-2.84 (m, 1H), 2.77-2.75 (m, 1H), 2.75-2.66 (m, 1H), 2.16-2.11 (m, 1H), 1.79 (q, J=7.3 Hz, 5H), 1.65-1.52 (m, 6H), 1.43 (s, 8H), 1.37-1.26 (m, 2H), 1.16 (br, 2H). HRMS (ESI) calcd for C₄₉H₆₂N₇O₆S⁺ [M+H]⁺, 876.4477; found, 876.4478.

### Example 53: Preparation of (E)-N-(4-(1-(4-(4-(2-((2-(2,6-dioxopiperidin-3-yl)-1,3-dioxoisoindolin-4-yl)thio)acetyl)piperazin-1-yl)benzoyl)piperidin-4-yl)butyl)-3-(pyridin-3-yl)acrylamide (SIAIS632027)

Referring to the methods of example 1 and Scheme 15, the compound (SIAIS632027) was prepared by using Daporinad derivative 1 (SIAIS630006) and intermediate LM (SIAIS151045; 2-((2-(2,6-dioxopiperidin-3-yl)-1,3-dioxoisoindolin-4-yl)thio)acetic acid; CAS No.: 2378582-26-2). The compound (SIAIS632027) was obtained as yellow solid (8.7 mg, yield 32%). ¹H NMR (500 MHz, CD₃OD) δ 9.01 (s, 1H), 8.77 (d, J=4.6 Hz, 1H), 8.71 (dt, J=8.3, 1.7 Hz, 1H), 8.02 (dd, J=8.3, 5.6 Hz, 1H), 7.85 (d, J=8.2 Hz, 1H), 7.77-7.70 (m, 1H), 7.69-7.60 (m, 2H), 7.33 (d, J=8.8 Hz, 2H), 7.02 (d, J=8.9 Hz, 2H), 6.91 (d, J=15.8 Hz, 1H), 5.12 (dd, J=12.7, 5.5 Hz, 1H), 4.20 (s, 2H), 3.79 (dt, J=42.2, 5.4 Hz, 5H), 3.38 (t, J=5.2 Hz, 2H), 3.34 (d, J=7.0 Hz, 3H), 3.27 (t, J=5.4 Hz, 2H), 2.93-2.83 (m, 2H), 2.79-2.66 (m, 3H), 2.18-2.10 (m, 1H), 1.76 (br, 2H), 1.59 (p, J=7.3 Hz, 3H), 1.47-1.39 (m, 2H), 1.36-1.31 (m, 2H), 1.21-1.10 (m, 2H). HRMS (ESI) calcd for C₄₃H₄₈N₇O₇S⁺ [M+H]⁺, 806.3330; found, 806.3335.

### Example 54: Preparation of (E)-N-(4-(1-(4-(4-(3-((2-(2,6-dioxopiperidin-3-yl)-1,3-dioxoisoindolin-4-yl)thio)propanoyl)piperazin-1-yl)benzoyl)piperidin-4-yl)butyl)-3-(pyridin-3-yl)acrylamide (SIAIS632028)

Referring to the methods of example 1 and Scheme 15, the compound (SIAIS632028) was prepared by using Daporinad derivative 1 (SIAIS630006) and intermediate LM (SIAIS151138B; 3-((2-(2,6-dioxopiperidin-3-yl)-1,3-dioxoisoindolin-4-yl)thio)propanoic acid; CAS No.: 2378582-27-3). The compound (SIAIS632028) was obtained as yellow solid (8.6 mg, yield 27%). ¹H NMR (500 MHz, DMSO-d₆) δ 11.12 (s, 1H), 9.03 (s, 1H), 8.79 (d, J=5.4 Hz, 1H), 8.53 (d, J=7.8 Hz, 1H), 8.34 (s, 1H), 7.95-7.85 (m, 1H), 7.85-7.76 (m, 2H), 7.64 (dd, J=6.3, 1.7 Hz, 1H), 7.54 (d, J=15.9 Hz, 1H), 7.26 (d, J=8.6 Hz, 2H), 6.96 (d, J=8.5 Hz, 2H), 6.90 (d, J=15.9 Hz, 1H), 5.11 (dd, J=12.9, 5.4 Hz, 1H), 3.60 (d, J=25.7 Hz, 6H), 3.35 (t, J=7.1 Hz, 2H), 3.25-3.15 (m, 6H), 2.95-2.80 (m, 5H), 2.63-2.55 (m, 1H), 2.52 (br, 1H), 2.07-1.96 (m, 1H), 1.66 (d, J=12.4 Hz, 2H), 1.51-1.40 (m, 3H), 1.35-1.20 (m, 4H), 1.10-0.95 (m, 2H). HRMS (ESI) calcd for C₄₄H₅₀N₇O₇S⁺ [M+H]⁺, 820.3487; found, 820.3489.

### Example 55: Preparation of (E)-N-(4-(1-(4-(4-(5-((2-(2,6-dioxopiperidin-3-yl)-1,3-dioxoisoindolin-4-yl)thio)pentanoyl)piperazin-1-yl)benzoyl)piperidin-4-yl)butyl)-3-(pyridin-3-yl)acrylamide (SIAIS632029)

Referring to the methods of example 1 and Scheme 15, the compound (SIAIS632029) was prepared by using Daporinad derivative 1 (SIAIS630006) and intermediate LM (SIAIS151140B; 5-((2-(2,6-dioxopiperidin-3-yl)-1,3-dioxoisoindolin-4-yl)thio)valeric acid; CAS No.: 2378582-29-5). The compound (SIAIS632029) was obtained as yellow solid (9.1 mg, yield 28%). ¹H NMR (500 MHz, DMSO-d₆) δ 11.12 (s, 1H), 8.97 (s, 1H), 8.74 (d, J=5.3 Hz, 1H), 8.43 (d, J=8.1 Hz, 1H), 8.29 (t, J=5.7 Hz, 1H), 7.83 (t, J=6.7 Hz, 1H), 7.80-7.75 (m, 2H), 7.62 (dd, J=6.3, 1.8 Hz, 1H), 7.52 (d, J=15.8 Hz, 1H), 7.26 (d, J=8.5 Hz, 2H), 6.96 (d, J=8.7 Hz, 2H), 6.86 (d, J=15.9 Hz, 1H), 5.11 (dd, J=12.8, 5.4 Hz, 1H), 3.63-3.57 (m, 5H), 3.29-3.12 (m, 8H), 2.96-2.81 (m, 3H), 2.64-2.67 (m, 1H), 2.54-2.51 (m, 1H), 2.45-2.41 (m, 2H), 2.08-2.00 (m, 1H), 1.73-1.67 (m, 7H), 1.51-1.43 (m, 3H), 1.36-1.28 (m, 2H), 1.26-1.20 (m, 2H), 1.10-0.98 (m, 2H). HRMS (ESI) calcd for C₄₆H₅₄N₇O₇S⁺ [M+H]⁺, 848.3800; found, 848.3806.

### Example 56: Preparation of (E)-N-(4-(1-(4-(4-(6-((2-(2,6-dioxopiperidin-3-yl)-1,3-dioxoisoindolin-4-yl)thio)hexanoyl)piperazin-1-yl)benzoyl)piperidin-4-yl)butyl)-3-(pyridin-3-yl)acrylamide (SIAIS632030)

Referring to the methods of example 1 and Scheme 15, the compound (SIAIS632030) was prepared by using Daporinad derivative 1 (SIAIS630006) and intermediate LM (SIAIS151141B; 6-((2-(2,6-dioxopiperidin-3-yl)-1,3-dioxoisoindolin-4-yl)thio)caproic acid; CAS No.: 2378582-30-8). The compound (SIAIS632030) was obtained as yellow solid (8.8 mg, yield 26%). ¹H NMR (500 MHz, DMSO-d₆) δ 11.12 (s, 1H), 9.01 (d, J=9.5 Hz, 1H), 8.78 (t, J=5.6 Hz, 1H), 8.55-8.45 (m, 1H), 8.37-8.29 (m, 1H), 7.97-7.86 (m, 1H), 7.83-7.72 (m, 2H), 7.61 (d, J=6.9 Hz, 1H), 7.54 (dd, J=15.9, 2.5 Hz, 1H), 7.26 (d, J=8.4 Hz, 2H), 6.97 (dd, J=8.9, 3.1 Hz, 2H), 6.94-6.86 (m, 1H), 5.11 (dd, J=12.9, 5.4 Hz, 1H), 3.59 (d, J=5.4 Hz, 5H), 3.25-3.17 (m, 6H), 3.13 (t, J=7.3 Hz, 2H), 2.95-2.82 (m, 3H), 2.63-2.57 (m, 1H), 2.53-2.51 (m, 1H), 2.37 (t, J=7.3 Hz, 2H), 2.08-2.03 (m, 1H), 1.75-1.62 (m, 4H), 1.56 (q, J=7.4 Hz, 2H), 1.51-1.45 (m, 5H), 1.36-1.18 (m, 4H), 1.08-0.97 (m, 2H). HRMS (ESI) calcd for C₄₇H₅₆N₇O₇S⁺ [M+H]⁺, 862.3956; found, 862.3959.

### Example 57: Preparation of (E)-N-(4-(1-(4-(4-(7-((2-(2,6-dioxopiperidin-3-yl)-1,3-dioxoisoindolin-4-yl)thio)heptanoyl)piperazin-1-yl)benzoyl)piperidin-4-yl)butyl)-3-(pyridin-3-yl)acrylamide (SIAIS632031)

Referring to the methods of example 1 and Scheme 15, the compound (SIAIS632031) was prepared by using Daporinad derivative 1 (SIAIS630006) and intermediate LM (SIAIS151142B; 7-((2-(2,6-dioxopiperidin-3-yl)-1,3-dioxoisoindolin-4-yl)thio)heptanoic acid; CAS No.: 2378582-31-9). The compound (SIAIS632031) was obtained as yellow solid (10.1 mg, yield 29%). ¹H NMR (500 MHz, DMSO-d₆) δ 11.12 (s, 1H), 9.00 (s, 1H), 8.76 (t, J=5.1 Hz, 1H), 8.47 (br, 1H), 8.31 (br, 1H), 7.87 (br, 1H), 7.76 (dt, J=15.9, 8.0 Hz, 2H), 7.62 (d, J=6.9 Hz, 1H), 7.53 (d, J=15.9 Hz, 1H), 7.26 (d, J=8.6 Hz, 2H), 6.96 (d, J=7.4 Hz, 1H), 6.91-6.82 (m, 1H), 5.11 (dd, J=12.8, 5.4 Hz, 1H), 3.58 (d, J=5.5 Hz, 5H), 3.25-3.15 (m, 6H), 3.13 (t, J=7.3 Hz, 2H), 2.95-2.79 (m, 3H), 2.62-2.57 (m, 1H), 2.53 (d, J=4.6 Hz, 1H), 2.35 (t, J=7.4 Hz, 2H), 2.08-2.04 (m, 1H), 1.66 (q, J=7.2 Hz, 4H), 1.56-1.42 (m, 8H), 1.40-1.28 (m, 4H), 1.26-1.22 (m, 2H), 1.08-1.00 (m, 2H). HRMS (ESI) calcd for C₄₈H₅₈N₇O₇S⁺ [M+H]⁺, 876.4113; found, 876.4116.

### Example 58: Preparation of (E)-N-(4-(1-(4-(4-(8-((2-(2,6-dioxopiperidin-3-yl)-1,3-dioxoisoindolin-4-yl)thio)octanoyl)piperazin-1-yl)benzoyl)piperidin-4-yl)butyl)-3-(pyridin-3-yl)acrylamide (SIAIS632032)

Referring to the methods of example 1 and Scheme 15, the compound (SIAIS632032) was prepared by using Daporinad derivative 1 (SIAIS630006) and intermediate LM (8-((2-(2,6-dioxopiperidin-3-yl)-1,3-dioxoisoindolin-4-yl)thio)octanoic acid; CAS No.: 2378585-62-5). The compound (SIAIS632032) was obtained as yellow solid (10 mg, yield 28%). ¹H NMR (500 MHz, DMSO-d₆) δ 11.12 (s, 1H), 9.03 (s, 1H), 8.80 (d, J=5.1 Hz, 1H), 8.55 (d, J=8.1 Hz, 1H), 8.34 (d, J=5.3 Hz, 1H), 7.98-7.90 (m, 1H), 7.84-7.69 (m, 2H), 7.62 (d, J=6.9 Hz, 1H), 7.54 (d, J=15.9 Hz, 1H), 7.26 (d, J=8.7 Hz, 2H), 6.98 (d, J=9.2 Hz, 2H), 6.90 (d, J=15.9 Hz, 1H), 5.11 (dd, J=12.9, 5.4 Hz, 1H), 3.59 (t, J=5.2 Hz, 5H), 3.26-3.17 (m, 5H), 3.12 (t, J=7.3 Hz, 2H), 2.94-2.79 (m, 3H), 2.64-2.56 (m, 1H), 2.53 (d, J=4.8 Hz, 1H), 2.34 (t, J=7.5 Hz, 2H), 2.09-2.00 (m, 1H), 1.72-1.58 (m, 4H), 1.55-1.41 (m, 8H), 1.37-1.19 (m, 8H), 1.09-1.00 (m, 2H). HRMS (ESI) calcd for C₄₉H₆₀N₇O₇S⁺ [M+H]⁺, 890.4269; found, 890.4275.

### Example 59: Preparation of (E)-N-(4-(1-(4-(4-(2-((2-(2,6-dioxopiperidin-3-yl)-1,3-dioxoisoindolin-4-yl)oxy)acetyl)piperazin-1-yl)benzoyl)piperidin-4-yl)butyl)-3-(pyridin-3-yl)acrylamide (SIAIS632033)

Referring to the methods of example 1 and Scheme 15, the compound (SIAIS632033) was prepared by using Daporinad derivative 1 (SIAIS630006) and intermediate LM (2-((2-(2,6-dioxopiperidin-3-yl)-1,3-dioxoisoindolin-4-yl)oxy)acetic acid; CAS No.: 1061605-21-7). The compound (SIAIS632033) was obtained as pale yellow solid (7.3 mg, yield 24%). ¹H NMR (500 MHz, DMSO-d₆) δ 11.11 (s, 1H), 9.01 (s, 1H), 8.78 (d, J=5.4 Hz, 1H), 8.51 (d, J=7.3 Hz, 1H), 8.33 (t, J=5.6 Hz, 1H), 7.90 (p, J=4.0 Hz, 1H), 7.78 (dd, J=8.6, 7.2 Hz, 1H), 7.54 (d, J=15.9 Hz, 1H), 7.45 (d, J=7.2 Hz, 1H), 7.38 (s, 1H), 7.27 (d, J=8.7 Hz, 2H), 6.99 (d, J=8.5 Hz, 2H), 6.89 (dd, J=15.8, 1.9 Hz, 1H), 5.24 (s, 2H), 5.10 (dd, J=12.8, 5.5 Hz, 1H), 3.61 (d, J=5.1 Hz, 5H), 3.41-3.12 (m, 7H), 2.95-2.78 (m, 3H), 2.64-2.57 (m, 1H), 2.54 (d, J=4.3 Hz, 1H), 2.06-1.99 (m, 1H), 1.67 (br, 2H), 1.52-1.40 (m, 3H), 1.36-1.28 (m, 2H), 1.28-1.20 (q, J=6.9 Hz, 2H), 1.08-1.00 (m, 2H). HRMS (ESI) calcd for C₄₃H₄₈N₇O₈⁺ [M+H]⁺, 790.3559; found, 790.3562.

### Example 60: Preparation of (E)-N-(4-(1-(4-(4-(5-((2-(2,6-dioxopiperidin-3-yl)-1,3-dioxoisoindolin-4-yl)oxy)pentanoyl)piperazin-1-yl)benzoyl)piperidin-4-yl)butyl)-3-(pyridin-3-yl)acrylamide (SIAIS632034)

Referring to the methods of example 1 and Scheme 15, the compound (SIAIS632034) was prepared by using Daporinad derivative 1 (SIAIS630006) and intermediate LM (5-((2-(2,6-dioxopiperidin-3-yl)-1,3-dioxoisoindolin-4-yl)oxy)pentanoic acid; CAS No.: 2169266-67-3). The compound (SIAIS632034) was obtained as pale yellow solid (8.7 mg, yield 26%). ¹H NMR (500 MHz, CD₃OD) δ 8.99 (d, J=2.0 Hz, 1H), 8.75 (d, J=5.5 Hz, 1H), 8.67 (dt, J=8.3, 1.7 Hz, 1H), 7.98 (dd, J=8.2, 5.5 Hz, 1H), 7.77 (dd, J=8.5, 7.3 Hz, 1H), 7.62 (d, J=15.8 Hz, 1H), 7.44 (dd, J=7.9, 6.7 Hz, 2H), 7.39-7.30 (m, 2H), 6.99 (d, J=8.9 Hz, 2H), 6.90 (d, J=15.9 Hz, 1H), 5.07 (dd, J=12.5, 5.5 Hz, 1H), 4.28 (t, J=5.7 Hz, 2H), 3.79-3.60 (m, 4H), 3.35-3.30 (m, 6H), 3.24-3.22 (m, 4H), 2.88-2.77 (m, 2H), 2.71-2.60 (m, 3H), 2.13-2.05 (m, 1H), 1.98-1.86 (m, 4H), 1.77 (br, 2H), 1.59 (p, J=7.2 Hz, 3H), 1.47-1.39 (m, 2H), 1.36-1.32 (m, 2H), 1.16 (br, 2H). HRMS (ESI) calcd for C₄₆H₅₄N₇O₈⁺ [M+H]⁺, 832.4028; found, 832.4029.

### Example 61: Preparation of (E)-N-(4-(1-(4-(4-(6-((2-(2,6-dioxopiperidin-3-yl)-1,3-dioxoisoindolin-4-yl)oxy)hexanoyl)piperazin-1-yl)benzoyl)piperidin-4-yl)butyl)-3-(pyridin-3-yl)acrylamide (SIAIS632035)

Referring to the methods of example 1 and Scheme 15, the compound (SIAIS632035) was prepared by using Daporinad derivative 1 (SIAIS630006) and intermediate LM (6-((2-(2,6-dioxopiperidin-3-yl)-1,3-dioxoisoindolin-4-yl)oxy)hexanoic acid; CAS No.: 2087490-48-8). The compound (SIAIS632035) was obtained as pale yellow solid (7.2 mg, yield 21%). ¹H NMR (500 MHz, DMSO-d6) δ 11.10 (s, 1H), 8.97 (d, J=10.8 Hz, 1H), 8.74 (q, J=5.5 Hz, 1H), 8.40 (s, 1H), 8.28 (d, J=7.5 Hz, 1H), 7.80 (dd, J=8.5, 7.2 Hz, 2H), 7.56-7.49 (m, 2H), 7.43 (d, J=7.2 Hz, 1H), 7.25 (d, J=8.4 Hz, 2H), 6.97-6.93 (m, 2H), 6.84 (d, J=15.1 Hz, 1H), 5.07 (dd, J=12.8, 5.5 Hz, 1H), 4.21 (t, J=6.3 Hz, 2H), 3.62-3.56 (m, 7H), 3.25-3.15 (m, 6H), 2.90-2.85 (m, 2H), 2.62-2.55 (m, 1H), 2.53-2.51 (m, 1H), 2.38 (t, J=7.4 Hz, 2H), 2.06-1.99 (m, 1H), 1.81-1.76 (m, 2H), 1.71-1.55 (m, 4H), 1.53-1.42 (m, 5H), 1.35-1.21 (m, 4H), 1.09-1.00 (m, 2H). HRMS (ESI) calcd for C₄₇H₅₆N₇O₈⁺ [M+H]⁺, 846.4185; found, 846.4189.

### Example 62: Preparation of (E)-N-(4-(1-(4-(4-(7-((2-(2,6-dioxopiperidin-3-yl)-1,3-dioxoisoindolin-4-yl)oxy)heptanoyl)piperazin-1-yl)benzoyl)piperidin-4-yl)butyl)-3-(pyridin-3-yl)acrylamide (SIAIS632036)

Referring to the methods of example 1 and Scheme 15, the compound (SIAIS632036) was prepared by using Daporinad derivative 1 (SIAIS630006) and intermediate LM (7-((2-(2,6-dioxopiperidin-3-yl)-1,3-dioxoisoindolin-4-yl)oxy)heptanoic acid; CAS No.: 2169266-69-5). The compound (SIAIS632036) was obtained as pale yellow solid (9.6 mg, yield 26%). ¹H NMR (500 MHz, DMSO-d6) δ 11.10 (s, 1H), 8.97 (d, J=5.4 Hz, 1H), 8.74 (d, J=5.1 Hz, 1H), 8.41 (d, J=8.5 Hz, 1H), 8.33-8.24 (m, 1H), 7.81 (dd, J=8.6, 7.3 Hz, 2H), 7.60-7.49 (m, 2H), 7.44 (d, J=7.3 Hz, 1H), 7.26 (d, J=8.7 Hz, 2H), 6.96 (d, J=8.4 Hz, 2H), 6.85 (d, J=15.9 Hz, 1H), 5.08 (dd, J=12.7, 5.4 Hz, 1H), 4.21 (t, J=6.4 Hz, 2H), 3.62-3.57 (m, 7H), 3.26-3.13 (m, 6H), 2.92-2.83 (m, 1H), 2.61-2.55 (m, 1H), 2.54-2.52 (m, 1H), 2.36 (t, J=7.4 Hz, 2H), 2.06-1.98 (m, 1H), 1.80-1.74 (m, 2H), 1.71-1.64 (m, 2H), 1.57-1.43 (m, 7H), 1.42-1.28 (m, 4H), 1.27-1.21 (m, 2H), 1.11-0.99 (m, 2H). HRMS (ESI) calcd for C₄₈H₅₈N₇O₈⁺ [M+H]⁺, 860.4341; found, 860.4346.

### Example 63: Preparation of (E)-N-(4-(1-(4-(1-(7-(2-(2,6-dioxopiperidin-3-yl)-1-oxoisoindolin-4-yl)hept-6-yn-1-yl)piperidin-4-yl)benzoyl)piperidin-4-yl)butyl)-3-(pyridin-3-yl)acrylamide (SIAIS631139)

Referring to the methods of example 15 and Scheme 16, the compound (SIAIS631139) was prepared by using Daporinad derivative 3 (SIAIS631127) and intermediate LM (SIAIS292017; 7-(2-(2,6-dioxopiperidin-3-yl)-1-oxoisoindolin-4-yl)hept-6-yn-1-yl methanesulfonate; CAS No.: 2570254-41-8). The compound (SIAIS631139) was obtained as white solid (7.7 mg, yield 23%). ¹H NMR (500 MHz, DMSO-d₆) δ 11.00 (s, 1H), 10.62 (s, 1H), 8.95 (d, J=2.3 Hz, 1H), 8.73 (d, J=5.5 Hz, 1H), 8.39 (d, J=8.1 Hz, 1H), 8.34-8.27 (m, 1H), 7.80 (dd, J=8.2, 5.3 Hz, 1H), 7.71 (d, J=7.5 Hz, 1H), 7.65 (d, J=7.6 Hz, 1H), 7.57-7.47 (m, 2H), 7.36-7.23 (m, 4H), 6.86 (d, J=15.9 Hz, 1H), 5.16 (dd, J=13.3, 5.1 Hz, 1H), 4.47 (d, J=17.7 Hz, 1H), 4.33 (d, J=17.7 Hz, 1H), 3.57-3.52 (m, 2H), 3.18 (q, J=6.6 Hz, 2H), 3.09-2.81 (m, 8H), 2.72 (br, 1H), 2.63-2.55 (m, 1H), 2.53 (t, J=7.1 Hz, 2H), 2.48-2.43 (m, 1H), 2.10 (d, J=12.6 Hz, 1H), 2.06-2.00 (m, 1H), 1.99-1.94 (m, 1H), 1.85-1.76 (m, 2H), 1.62 (q, J=7.2 Hz, 3H), 1.53-1.43 (m, 5H), 1.35-1.22 (m, 4H), 1.05 (br, 2H). HRMS (ESI) calcd for C₄₉H₅₉N₆O₅⁺ [M+H]⁺, 811.4541; found, 811.4544.

### Example 64: Preparation of (E)-N-(4-(1-(4-(1-(8-(2-(2,6-dioxopiperidin-3-yl)-1-oxoisoindolin-4-yl)oct-7-yn-1-yl)piperidin-4-yl)benzoyl)piperidin-4-yl)butyl)-3-(pyridin-3-yl)acrylamide (SIAIS631140)

Referring to the methods of example 15 and Scheme 16, the compound (SIAIS631140) was prepared by using Daporinad derivative 3 (SIAIS631127) and intermediate LM (SIAIS292020; 8-(2-(2,6-dioxopiperidin-3-yl)-1-oxoisoindolin-4-yl)oct-7-yn-1-yl methanesulfonate; CAS No.: 2570254-42-9). The compound (SIAIS631140) was obtained as white solid (10.1 mg, yield 31%). ¹H NMR (500 MHz, DMSO-d6) δ 11.01 (s, 1H), 8.90 (s, 1H), 8.69 (s, 1H), 8.27 (s, 2H), 7.71 (d, J=7.5 Hz, 2H), 7.64 (d, J=7.5 Hz, 1H), 7.56-7.47 (m, 2H), 7.36-7.25 (m, 4H), 6.82 (d, J=16.2 Hz, 1H), 5.16 (dd, J=13.3, 5.1 Hz, 1H), 4.46 (d, J=17.7 Hz, 1H), 4.33 (d, J=17.7 Hz, 1H), 3.59-3.51 (m, 2H), 3.18 (q, J=6.6 Hz, 2H), 3.09-2.80 (m, 8H), 2.73 (br, 1H), 2.65-2.57 (m, 1H), 2.52 (br, 2H), 2.49-2.42 (m, 1H), 2.12-2.01 (m, 2H), 1.97 (d, J=13.6 Hz, 1H), 1.76 (br, 2H), 1.61 (p, J=7.1 Hz, 3H), 1.53-1.42 (m, 5H), 1.40-1.27 (m, 4H), 1.27-1.21 (m, 2H), 1.05 (br, 2H). HRMS (ESI) calcd for C₅₀H₆₁N₆O₅⁺ [M+H]⁺, 825.4698; found, 825.4702.

### Example 65: Preparation of (E)-N-(4-(1-(4-(1-(4-((2-(2,6-dioxopiperidin-3-yl)-1-oxoisoindolin-4-yl)thio)butyl)piperidin-4-yl)benzoyl)piperidin-4-yl)butyl)-3-(pyridin-3-yl)acrylamide (SIAIS631141)

Referring to the methods of example 15 and Scheme 16, the compound (SIAIS631141) was prepared by using Daporinad derivative 3 (SIAIS631127) and intermediate LM (SIAIS213134; 3-(4-(4-bromobutylthio)-1-oxoisoindolin-2-yl)piperidine-2,6-dione; CAS No.: 2378582-59-1). The compound (SIAIS631141) was obtained as white solid (9.7 mg, yield 30%). ¹H NMR (500 MHz, CD₃OD) δ 8.96 (s, 1H), 8.72 (s, 1H), 8.58 (d, J=8.2 Hz, 1H), 7.91 (dd, J=8.2, 5.4 Hz, 1H), 7.77-7.64 (m, 2H), 7.63-7.52 (m, 2H), 7.37 (s, 4H), 6.90 (d, J=15.9 Hz, 1H), 5.18 (dd, J=13.4, 5.2 Hz, 1H), 4.50 (d, J=17.4 Hz, 1H), 4.43 (d, J=17.4 Hz, 1H), 3.75-3.63 (m, 4H), 3.34 (d, J=7.0 Hz, 2H), 3.23-3.04 (m, 7H), 3.02-2.91 (m, 1H), 2.86-2.76 (m, 1H), 2.59-2.51 (m, 1H), 2.25-2.17 (m, 1H), 2.14-1.93 (m, 7H), 1.87 (br, 1H), 1.78-1.72 (m, 2H), 1.71-1.64 (m, 1H), 1.59 (p, J=7.2 Hz, 4H), 1.47-1.38 (m, 3H), 1.37-1.28 (m, 3H), 1.26-1.08 (m, 2H). HRMS (ESI) calcd for C₄₆H₅₇N₆O₅S+⁺ [M+H]⁺, 805.4106; found, 805.4107.

### Example 66: Preparation of (E)-N-(4-(1-(6-(4-(4-(7-(2-(2,6-dioxopiperidin-3-yl)-1-oxoisoindolin-4-yl)hept-6-yn-1-yl)piperazin-1-yl)piperidin-1-yl)pyridazine-3-carbonyl)piperidin-4-yl)butyl)-3-(pyridin-3-yl)acrylamide (SIAIS632005)

Referring to the methods of example 15 and Scheme 16, the compound (SIAIS631139) was prepared by using Daporinad derivative 5 (SIAIS631135) and intermediate LM (SIAIS292017; 7-(2-(2,6-dioxopiperidin-3-yl)-1-oxoisoindolin-4-yl)hept-6-yn-1-yl methanesulfonate; CAS No.: 2570254-41-8). The compound (SIAIS631139) was obtained as white solid (8.3 mg, yield 24%). ¹H NMR (500 MHz, DMSO-d₆) δ 10.99 (s, 1H), 9.00 (s, 1H), 8.76 (s, 1H), 8.48 (s, 1H), 8.34 (s, 1H), 7.88 (br, 1H), 7.71 (d, J=7.6 Hz, 1H), 7.65 (dd, J=7.7, 1.1 Hz, 2H), 7.58 (br, 1H), 7.55-7.53 (m, 1H), 7.51 (d, J=7.4 Hz, 1H), 6.90 (d, J=15.2 Hz, 1H), 5.15 (dd, J=13.3, 5.1 Hz, 1H), 4.62 (d, J=13.1 Hz, 2H), 4.47 (d, J=17.7 Hz, 2H), 4.33 (d, J=17.7 Hz, 1H), 3.94 (d, J=13.0 Hz, 2H), 3.72 (br, 5H), 3.52 (br, 2H), 3.25-2.98 (m, 8H), 2.96-2.87 (m, 1H), 2.78 (t, J=12.6 Hz, 1H), 2.67-2.55 (m, 1H), 2.52 (d, J=7.3 Hz, 2H), 2.49-2.46 (m, 1H), 2.23 (d, J=11.6 Hz, 2H), 2.05-1.97 (m, 1H), 1.82-1.72 (m, 5H), 1.67-1.54 (m, 4H), 1.53-1.43 (m, 4H), 1.36-1.29 (m, 2H), 1.28-1.22 (m, 2H), 1.16-1.05 (m, 2H). HRMS (ESI) calcd for C₅₁H₆₅N₁₀O₅⁺ [M+H]⁺, 897.5134; found, 897.5138.

### Example 67: Preparation of (E)-N-(4-(1-(6-(4-(4-(8-(2-(2,6-dioxopiperidin-3-yl)-1-oxoisoindolin-4-yl)oct-7-yn-1-yl)piperazin-1-yl)piperidin-1-yl)pyridazine-3-carbonyl)piperidin-4-yl)butyl)-3-(pyridin-3-yl)acrylamide (SIAIS632006)

Referring to the methods of example 15 and Scheme 16, the compound (SIAIS632006) was prepared by using Daporinad derivative 5 (SIAIS631135) and intermediate LM (SIAIS292020; 8-(2-(2,6-dioxopiperidin-3-yl)-1-oxoisoindolin-4-yl)oct-7-yn-1-yl methanesulfonate; CAS No.: 2570254-42-9). The compound (SIAIS632006) was obtained as white solid (9.3 mg, yield 26%). ¹H NMR (500 MHz, DMSO-d₆) δ 11.00 (s, 1H), 8.97 (s, 1H), 8.74 (s, 1H), 8.38 (d, J=49.8 Hz, 2H), 7.83 (br, 1H), 7.71 (d, J=7.6 Hz, 1H), 7.67-7.59 (m, 2H), 7.56-7.48 (m, 3H), 6.88 (d, J=16.3 Hz, 1H), 5.15 (dd, J=13.3, 5.1 Hz, 1H), 4.62 (d, J=13.1 Hz, 2H), 4.47 (dd, J=16.0, 10.9 Hz, 2H), 4.32 (d, J=17.6 Hz, 1H), 3.95 (d, J=13.5 Hz, 2H), 3.73 (br, 5H), 3.52 (br, 2H), 3.23-2.98 (m, 8H), 2.95-2.85 (m, 1H), 2.83-2.74 (m, 1H), 2.70-2.58 (m, 1H), 2.49 (br, 2H), 2.47-2.43 (m, 1H), 2.22 (br, 2H), 2.10-1.96 (m, 1H), 1.83-1.70 (m, 5H), 1.67-1.50 (m, 4H), 1.47 (p, J=7.2 Hz, 4H), 1.37-1.29 (m, 4H), 1.28-1.21 (m, 2H), 1.18-1.05 (m, 2H). HRMS (ESI): calcd for C₅₂H₆₇N₁₀O₅⁺ [M+H]⁺, 911.5290; found, 911.5294.

### Example 68: Preparation of (E)-N-(4-(1-(6-(4-(4-(4-((2-(2,6-dioxopiperidin-3-yl)-1-oxoisoindolin-4-yl)thio)butyl)piperazin-1-yl)piperidin-1-yl)pyridazine-3-carbonyl)piperidin-4-yl)butyl)-3-(pyridin-3-yl)acrylamide (SIAIS632007)

Referring to the methods of example 15 and Scheme 16, the compound (SIAIS632007) was prepared by using Daporinad derivative 5 (SIAIS631135) and intermediate LM (SIAIS213134; 3-(4-(4-bromobutylthio)-1-oxoisoindolin-2-yl)piperidine-2,6-dione; CAS No.: 2378582-59-1). The compound (SIAIS632007) was obtained as white solid (9.7 mg, yield 28%). ¹H NMR (500 MHz, DMSO-d₆) δ 10.99 (s, 1H), 8.99 (s, 1H), 8.76 (d, J=5.6 Hz, 1H), 8.46 (s, 1H), 8.34 (s, 1H), 7.91-7.76 (m, 1H), 7.71-7.61 (m, 2H), 7.62-7.48 (m, 4H), 6.89 (d, J=15.9 Hz, 1H), 5.14 (dd, J=13.3, 5.2 Hz, 1H), 4.62 (d, J=13.1 Hz, 2H), 4.48 (d, J=12.9 Hz, 1H), 4.39 (d, J=17.4 Hz, 1H), 4.25 (d, J=17.4 Hz, 1H), 3.95 (d, J=13.2 Hz, 2H), 3.65 (br, 7H), 3.22-3.12 (m, 6H), 3.03 (t, J=13.0 Hz, 2H), 2.95-2.86 (m, 1H), 2.78 (t, J=12.6 Hz, 1H), 2.63-2.56 (m, 1H), 2.55-2.51 (m, 2H), 2.49-2.43 (m, 1H), 2.23 (d, J=11.6 Hz, 2H), 2.05-1.98 (m, 1H), 1.87 (t, J=8.0 Hz, 2H), 1.77 (br, 3H), 1.68-1.62 (m, 3H), 1.54 (br, 1H), 1.46 (q, J=7.2 Hz, 2H), 1.38-1.30 (m, 2H), 1.28-1.22 (m, 2H), 1.16-1.06 (m, 2H). HRMS (ESI) calcd for C₄₈H₆₃N₁₀O₅S⁺ [M+H]⁺, 891.4698; found, 891.4603.

### Example 69: Preparation of (E)-N-(4-(1-(6-(4-(7-(2-(2,6-dioxopiperidin-3-yl)-1-oxoisoindolin-4-yl)hept-6-yn-1-yl)piperazin-1-yl)pyridazine-3-carbonyl)piperidin-4-yl)butyl)-3-(pyridin-3-yl)acrylamide (SIAIS632010)

Referring to the methods of example 15 and Scheme 16, the compound (SIAIS632010) was prepared by using Daporinad derivative 4 (SIAIS632004) and intermediate LM (SIAIS292017; 7-(2-(2,6-dioxopiperidin-3-yl)-1-oxoisoindolin-4-yl)hept-6-yn-1-yl methanesulfonate; CAS No.: 2570254-41-8). The compound (SIAIS632010) was obtained as white solid (7.7 mg, yield 24%). ¹H NMR (500 MHz, CD₃OD) δ 8.94 (s, 1H), 8.71 (s, 1H), 8.53 (d, J=8.2 Hz, 1H), 7.88 (d, J=7.2 Hz, 1H), 7.75 (d, J=6.6 Hz, 1H), 7.66 (d, J=9.5 Hz, 1H), 7.64-7.57 (m, 2H), 7.51 (t, J=7.7 Hz, 1H), 7.45 (d, J=9.5 Hz, 1H), 6.87 (d, J=15.8 Hz, 1H), 5.19 (dd, J=13.3, 5.2 Hz, 1H), 4.63 (d, J=13.4 Hz, 1H), 4.53 (d, J=17.5 Hz, 1H), 4.47 (d, J=17.5 Hz, 1H), 3.93 (d, J=13.2 Hz, 2H), 3.71 (s, 2H), 3.51-3.37 (m, 2H), 3.34 (d, J=7.1 Hz, 2H), 3.27-3.22 (m, 3H), 3.20-3.13 (m, 2H), 2.95-2.87 (m, 2H), 2.85-2.76 (m, 1H), 2.57 (t, J=6.9 Hz, 2H), 2.54-2.47 (m, 1H), 2.25-2.15 (m, 1H), 1.95-1.86 (m, 3H), 1.79-1.70 (m, 3H), 1.65-1.55 (m, 5H), 1.46-1.40 (m, 2H), 1.39-1.30 (m, 2H), 1.29-1.16 (m, 3H). HRMS (ESI) calcd for C₄₆H₅₆N₉O₅⁺ [M+H]⁺, 814.4399; found, 814.4403.

### Example 70: Preparation of (E)-N-(4-(1-(6-(4-(8-(2-(2,6-dioxopiperidin-3-yl)-1-oxoisoindolin-4-yl)oct-7-yn-1-yl)piperazin-1-yl)pyridazine-3-carbonyl)piperidin-4-yl)butyl)-3-(pyridin-3-yl)acrylamide (SIAIS632011)

Referring to the methods of example 15 and Scheme 16, the compound (SIAIS632011) was prepared by using Daporinad derivative 4 (SIAIS632004) and intermediate LM (SIAIS292020; 8-(2-(2,6-dioxopiperidin-3-yl)-1-oxoisoindolin-4-yl)oct-7-yn-1-yl methanesulfonate; CAS No.: 2570254-42-9). The compound (SIAIS632011) was obtained as white solid (9.2 mg, yield 28%). ¹H NMR (500 MHz, CD₃OD) δ 9.06 (d, J=1.8 Hz, 1H), 8.82-8.79 (m, 2H), 8.09 (dd, J=7.8, 6.0 Hz, 1H), 7.75 (dd, J=7.7, 1.0 Hz, 1H), 7.70 (d, J=9.5 Hz, 1H), 7.66-7.60 (m, 2H), 7.53-7.49 (m, 2H), 6.95 (d, J=15.8 Hz, 1H), 5.19 (dd, J=13.3, 5.2 Hz, 1H), 4.63 (d, J=12.5 Hz, 2H), 4.53 (d, J=17.5 Hz, 1H), 4.47 (d, J=17.4 Hz, 1H), 3.93 (d, J=13.3 Hz, 1H), 3.72 (s, 2H), 3.54-3.42 (m, 2H), 3.36-3.34 (m, 3H), 3.25-3.19 (m, 3H), 3.18-3.14 (m, 1H), 2.96-2.87 (m, 3H), 2.85-2.76 (m, 1H), 2.59-2.45 (m, 3H), 2.25-2.15 (m, 1H), 1.93-1.81 (m, 3H), 1.78-1.66 (m, 3H), 1.65-1.59 (m, 5H), 1.54-1.47 (m, 2H), 1.47-1.40 (m, 2H), 1.37-1.31 (m, 2H), 1.31-1.20 (m, 3H). HRMS (ESI) calcd for C₄₇H₅₈N₉O₅⁺ [M+H]⁺, 828.4555; found, 828.4559.

### Example 71: Preparation of (E)-N-(4-(1-(6-(4-(4-((2-(2,6-dioxopiperidin-3-yl)-1-oxoisoindolin-4-yl)thio)butyl)piperazin-1-yl)pyridazine-3-carbonyl)piperidin-4-yl)butyl)-3-(pyridin-3-yl)acrylamide (SIAIS632012)

Referring to the methods of example 15 and Scheme 16, the compound (SIAIS632012) was prepared by using Daporinad derivative 4 (SIAIS632004) and intermediate LM (SIAIS213134; 3-(4-(4-bromobutylthio)-1-oxoisoindolin-2-yl)piperidine-2,6-dione; CAS No.: 2378582-59-1). The compound (SIAIS632012) was obtained as white solid (8.7 mg, yield 27%). ¹H NMR (500 MHz, CD₃OD) δ 9.04 (s, 1H), 8.81-8.75 (m, 2H), 8.07 (dd, J=8.2, 5.6 Hz, 1H), 7.73-7.61 (m, 4H), 7.56 (t, J=7.6 Hz, 1H), 7.48 (d, J=9.5 Hz, 1H), 6.93 (d, J=15.8 Hz, 1H), 5.19 (dd, J=13.4, 5.2 Hz, 1H), 4.63 (d, J=13.1 Hz, 2H), 4.50 (d, J=17.4 Hz, 1H), 4.43 (d, J=17.1 Hz, 1H), 3.94 (d, J=13.7 Hz, 1H), 3.67 (s, 1H), 3.48-3.41 (m, 1H), 3.34 (d, J=7.7 Hz, 3H), 3.24-3.12 (m, 6H), 2.96-2.88 (m, 2H), 2.84-2.77 (m, 1H), 2.61-2.49 (m, 1H), 2.22-2.19 (m, 1H), 2.00-1.86 (m, 3H), 1.78-1.70 (m, 3H), 1.64-1.58 (m, 3H), 1.50-1.41 (m, 2H), 1.40-1.34 (m, 3H), 1.33-1.22 (m, 4H). HRMS (ESI) calcd for C₄₃H₅₄N₉O₅S⁺ [M+H]⁺, 808.3963; found, 808.3967.

### Example 72: Preparation of (E)-N-(4-(1-(6-(4-(7-(2-(2,6-dioxopiperidin-3-yl)-1-oxoisoindolin-4-yl)hept-6-yn-1-yl)piperazin-1-yl)nicotinoyl)piperidin-4-yl)butyl)-3-(pyridin-3-yl)acrylamide (SIAIS632039)

Referring to the methods of example 15 and Scheme 16, the compound (SIAIS632039) was prepared by using Daporinad derivative 6 (SIAIS632025) and intermediate LM (SIAIS292017; 7-(2-(2,6-dioxopiperidin-3-yl)-1-oxoisoindolin-4-yl)hept-6-yn-1-yl methanesulfonate; CAS No.: 2570254-41-8). The compound (SIAIS632039) was obtained as white solid (6.5 mg, 20%). ¹H NMR (500 MHz, CD₃OD) δ 8.93 (s, 1H), 8.70 (s, 1H), 8.52 (d, J=8.2 Hz, 1H), 8.25 (d, J=2.3 Hz, 1H), 7.87 (dd, J=8.2, 5.4 Hz, 1H), 7.75 (d, J=7.7 Hz, 1H), 7.69 (dd, J=8.8, 2.4 Hz, 1H), 7.64-7.58 (m, 2H), 7.51 (t, J=7.7 Hz, 1H), 6.96 (d, J=8.8 Hz, 1H), 6.86 (d, J=15.8 Hz, 1H), 5.19 (dd, J=13.4, 5.2 Hz, 1H), 4.62-4.51 (m, 3H), 4.47 (d, J=17.4 Hz, 1H), 3.85-3.60 (m, 2H), 3.34 (d, J=7.3 Hz, 6H), 3.26-3.20 (m, 3H), 3.19-3.13 (m, 2H), 2.96-2.87 (m, 1H), 2.84-2.78 (m, 1H), 2.57 (t, J=6.9 Hz, 3H), 2.26-2.10 (m, 1H), 1.88-1.85 (m, 3H), 1.75 (q, J=7.3 Hz, 2H), 1.63-1.59 (m, 5H), 1.50-1.39 (m, 2H), 1.36-1.32 (m, 4H), 1.32-1.28 (m, 2H). HRMS (ESI) calcd for C₄₇H₅₇N₈O₅⁺ [M+H]⁺, 813.4446; found, 813.4449.

### Example 73: Preparation of (E)-N-(4-(1-(6-(4-(8-(2-(2,6-dioxopiperidin-3-yl)-1-oxoisoindolin-4-yl)oct-7-yn-1-yl)piperazin-1-yl)nicotinoyl)piperidin-4-yl)butyl)-3-(pyridin-3-yl)acrylamide (SIAIS632040)

Referring to the methods of example 15 and Scheme 16, the compound (SIAIS632040) was prepared by using Daporinad derivative 6 (SIAIS632025) and intermediate LM (SIAIS292020; 8-(2-(2,6-dioxopiperidin-3-yl)-1-oxoisoindolin-4-yl)oct-7-yn-1-yl methanesulfonate; CAS No.: 2570254-42-9). The compound (SIAIS632040) was obtained as white solid (7.4 mg, 22%). ¹H NMR (500 MHz, CD₃OD) δ 9.07 (s, 1H), 8.87-8.78 (m, 2H), 8.24 (d, J=2.3 Hz, 1H), 8.10 (dd, J=8.1, 5.8 Hz, 1H), 7.79-7.72 (m, 2H), 7.67-7.59 (m, 2H), 7.51 (t, J=7.7 Hz, 1H), 7.05 (d, J=9.0 Hz, 1H), 6.96 (d, J=15.9 Hz, 1H), 5.18 (dd, J=13.3, 5.2 Hz, 1H), 4.62-4.50 (m, 3H), 4.47 (d, J=17.5 Hz, 1H), 3.75-3.63 (m, 2H), 3.40-3.32 (m, 6H), 3.24-3.16 (m, 5H), 2.97-2.87 (m, 1H), 2.83-2.75 (m, 1H), 2.53 (t, J=6.8 Hz, 3H), 2.24-2.15 (m, 1H), 1.91-1.75 (m, 4H), 1.72-1.68 (m, 2H), 1.65-1.56 (m, 5H), 1.52-1.40 (m, 4H), 1.39-1.28 (m, 3H), 1.21-1.14 (m, 2H). HRMS (ESI) calcd for C₄₈H₅₉N₈O₅⁺ [M+H]⁺, 827.4603; found, 827.4608.

### Example 74: Preparation of (E)-N-(4-(1-(6-(4-(4-((2-(2,6-dioxopiperidin-3-yl)-1-oxoisoindolin-4-yl)thio)butyl)piperazin-1-yl)nicotinoyl)piperidin-4-yl)butyl)-3-(pyridin-3-yl)acrylamide (SIAIS632041)

Referring to the methods of example 15 and Scheme 16, the compound (SIAIS632041) was prepared by using Daporinad derivative 6 (SIAIS632025) and intermediate LM (SIAIS213134; 3-(4-(4-bromobutylthio)-1-oxoisoindolin-2-yl)piperidine-2,6-dione; CAS No.: 2378582-59-1). The compound (SIAIS632041) was obtained as white solid (7.6 mg, 24%). ¹H NMR (500 MHz, CD₃OD) δ 9.05 (s, 1H), 8.87-8.71 (m, 2H), 8.24 (d, J=2.3 Hz, 1H), 8.07 (dd, J=8.2, 5.7 Hz, 1H), 7.74-7.66 (m, 3H), 7.63 (d, J=15.8 Hz, 1H), 7.55 (t, J=7.7 Hz, 1H), 7.01 (d, J=8.9 Hz, 1H), 6.95 (d, J=15.9 Hz, 1H), 5.18 (dd, J=13.3, 5.2 Hz, 1H), 4.59-4.47 (m, 3H), 4.43 (d, J=17.4 Hz, 1H), 3.86 (s, 1H), 3.71-3.56 (m, 2H), 3.34 (t, J=7.2 Hz, 6H), 3.24-3.07 (m, 6H), 3.25-3.10 (m, 1H), 2.97-2.86 (m, 1H), 2.60-2.50 (m, 1H), 2.25-2.15 (m, 1H), 1.96 (p, J=7.3 Hz, 2H), 1.83-1.69 (m, 4H), 1.60 (p, J=7.3 Hz, 3H), 1.48-1.27 (m, 5H), 1.23-1.10 (m, 2H). HRMS (ESI) calcd for C₄₄H₅₅N₈O₅S⁺ [M+H]⁺, 807.4011; found, 807.4016.

### Example 75: Preparation of (E)-N-(4-(1-(4-(4-(3-(2-((2-(2,6-dioxopiperidin-3-yl)-1,3-dioxoisoindolin-4-yl)amino)ethoxy)propanoyl)piperazin-1-yl)benzoyl)piperidin-4-yl)butyl)-3-(pyridin-3-yl)acrylamide (SIAIS631079)

Referring to the methods of example 1 and Scheme 15, the compound (SIAIS631079) was prepared by using Daporinad derivative 1 (SIAIS630006) and intermediate LM (SIAIS151001; 3-(2-((2-(2,6-dioxopiperidin-3-yl)-1,3-dioxoisoindolin-4-yl)amino)ethoxy)propanoic acid; CAS No.: 2139348-60-8). The compound (SIAIS631079) was obtained as yellow solid (8.9 mg, yield 27%). ¹H NMR (500 MHz, CD₃OD) δ 8.94 (s, 1H), 8.70 (d, J=4.1 Hz, 1H), 8.55 (dt, J=8.2, 1.8 Hz, 1H), 7.89 (dd, J=8.2, 5.4 Hz, 1H), 7.60 (d, J=15.9 Hz, 1H), 7.51 (dd, J=8.6, 7.0 Hz, 1H), 7.25 (d, J=8.8 Hz, 2H), 7.02 (dd, J=15.3, 7.8 Hz, 2H), 6.88 (dd, J=12.4, 3.5 Hz, 3H), 5.01 (dd, J=12.5, 5.4 Hz, 1H), 3.82 (t, J=5.9 Hz, 2H), 3.79-3.66 (m, 6H), 3.52-3.43 (m, 3H), 3.40-3.33 (m, 5H), 3.26-3.15 (m, 4H), 2.81-2.75 (m, 1H), 2.75-2.68 (m, 3H), 2.67-2.59 (m, 1H), 2.07-1.99 (m, 1H), 1.77 (br, 2H), 1.59 (p, J=7.1 Hz, 3H), 1.47-1.39 (m, 2H), 1.36-1.32 (m, 2H), 1.16 (br, 2H). HRMS (ESI) calcd for C₄₃H₄₈N₇O₇S⁺ [M+H]⁺, 847.4137; found, 847.4139.

### Example 76: Preparation of (E)-N-(4-(1-(4-(4-(3-(2-(2-((2-(2,6-dioxopiperidin-3-yl)-1,3-dioxoisoindolin-4-yl)amino)ethoxy)ethoxy)propanoyl)piperazin-1-yl)benzoyl)piperidin-4-yl)butyl)-3-(pyridin-3-yl)acrylamide (SIAIS631109)

Referring to the methods of example 1 and Scheme 15, the compound (SIAIS631109) was prepared by using Daporinad derivative 1 (SIAIS630006) and intermediate LM (SIAIS151004; 3-(2-(2-((2-(2,6-dioxopiperidin-3-yl)-1,3-dioxoisoindolin-4-yl)amino)ethoxy)ethoxy)propanoic acid; CAS No.: 2140807-17-4). The compound (SIAIS631109) was obtained as yellow solid (9.6 mg, yield 27%). ¹H NMR (500 MHz, CD₃OD) δ 9.02 (s, 1H), 8.76 (d, J=21.8 Hz, 2H), 8.04 (s, 1H), 7.63 (d, J=15.6 Hz, 1H), 7.56 (dd, J=8.6, 7.1 Hz, 2H), 7.36 (d, J=8.7 Hz, 1H), 7.28 (d, J=8.7 Hz, 1H), 7.10 (d, J=8.6 Hz, 1H), 7.06 (d, J=7.3 Hz, 1H), 6.97-6.89 (m, 2H), 5.05 (dd, J=12.4, 5.5 Hz, 1H), 3.77-3.69 (m, 7H), 3.67-3.60 (m, 8H), 3.50 (t, J=5.3 Hz, 3H), 3.47-3.42 (m, 1H), 3.38 (dd, J=6.6, 3.9 Hz, 2H), 3.27-3.20 (m, 1H), 2.88-2.81 (m, 2H), 2.76 (dt, J=5.3, 2.7 Hz, 1H), 2.75-2.72 (m, 1H), 2.72-2.68 (m, 1H), 2.54 (t, J=6.3 Hz, 3H), 2.18-2.03 (m, 1H), 1.59 (p, J=7.1 Hz, 3H), 1.42 (br, 2H), 1.39-1.28 (m, 3H), 1.25-1.09 (m, 2H). HRMS (ESI) calcd for C₄₈H₅₉N₈O₉⁺ [M+H]⁺, 891.4400; found, 891.4403.

### Example 77: Preparation of (E)-N-(4-(1-(4-(4-(5-(2-(2,6-dioxopiperidin-3-yl)-1,3-dioxoisoindolin-4-yl)pent-4-yn-1-yl)piperazin-1-yl)benzoyl)piperidin-4-yl)butyl)-3-(pyridin-3-yl)acrylamide (SIAIS631119)

Referring to the methods of example 15 and Scheme 16, the compound (SIAIS631119) was prepared by using Daporinad derivative 1 (SIAIS630006) and intermediate LM (SIAIS292006; 5-(2-(2,6-dioxopiperidin-3-yl)-1,3-dioxoisoindolin-4-yl)pent-4-yn-1-yl methanesulfonate; CAS No.: 2570254-45-2). The compound (SIAIS631119) was obtained as white solid (8.7 mg, yield 28%). ¹H NMR (500 MHz, DMSO-d₆) δ 11.14 (s, 1H), 11.08 (s, 1H), 8.88 (d, J=2.2 Hz, 1H), 8.67 (dd, J=5.1, 1.5 Hz, 1H), 8.26 (q, J=8.2, 6.9 Hz, 2H), 8.00-7.88 (m, 3H), 7.68 (dd, J=8.1, 5.0 Hz, 1H), 7.49 (d, J=15.9 Hz, 1H), 7.29 (d, J=8.7 Hz, 2H), 7.02 (d, J=8.9 Hz, 2H), 6.82 (d, J=15.8 Hz, 1H), 5.16 (dd, J=12.9, 5.4 Hz, 1H), 3.92 (d, J=12.4 Hz, 2H), 3.62 (d, J=11.5 Hz, 2H), 3.34-3.14 (m, 8H), 2.94-2.85 (m, 1H), 2.67 (t, J=6.9 Hz, 2H), 2.63-2.58 (m, 1H), 2.57-2.52 (m, 1H), 2.15-2.01 (m, 3H), 1.67 (br, 2H), 1.46 (q, J=7.3 Hz, 3H), 1.35-1.18 (m, 5H), 1.10-1.00 (m, 2H). HRMS (ESI) calcd for C₄₆H₅₂N₇O₆⁺ [M+H]⁺, 798.3974; found, 798.3977.

### Example 78: Preparation of (E)-N-(4-(1-(4-(4-(6-(2-(2,6-dioxopiperidin-3-yl)-1,3-dioxoisoindolin-4-yl)hex-5-yn-1-yl)piperazin-1-yl)benzoyl)piperidin-4-yl)butyl)-3-(pyridin-3-yl)acrylamide (SIAIS631120)

Referring to the methods of example 15 and Scheme 16, the compound (SIAIS631120) was prepared by using Daporinad derivative 1 (SIAIS630006) and intermediate LM (SIAIS292007; 6-(2-(2,6-dioxopiperidin-3-yl)-1,3-dioxoisoindolin-4-yl)hex-5-yn-1-yl methanesulfonate; CAS No.: 2641288-61-9). The compound (SIAIS631120) was obtained as white solid (8.9 mg, yield 28%). ¹H NMR (500 MHz, CD₃OD) δ 8.72 (s, 1H), 8.51 (d, J=3.8 Hz, 1H), 8.19 (d, J=8.2 Hz, 1H), 7.78-7.72 (m, 3H), 7.57 (dd, J=8.1, 5.1 Hz, 1H), 7.47 (d, J=15.9 Hz, 1H), 7.26 (d, J=8.7 Hz, 2H), 6.97 (d, J=8.7 Hz, 2H), 6.70 (d, J=15.9 Hz, 1H), 5.05 (dd, J=12.8, 5.5 Hz, 1H), 4.46 (s, 1H), 3.87 (s, 2H), 3.72 (s, 1H), 3.61 (s, 2H), 3.23 (d, J=7.2 Hz, 2H), 3.22-3.16 (m, 4H), 3.10-3.02 (m, 2H), 2.82-2.73 (m, 2H), 2.71-2.60 (m, 2H), 2.54 (t, J=6.8 Hz, 2H), 2.09-2.02 (m, 1H), 1.98-1.87 (m, 2H), 1.67 (p, J=7.1 Hz, 4H), 1.49 (p, J=7.4 Hz, 4H), 1.35-1.27 (m, 2H), 1.28-1.17 (m, 2H), 1.07 (br, 2H). HRMS (ESI) calcd for C₄₇H₅₄N₇O₆⁺ [M+H]⁺, 812.4130; found, 812.4132.

### Example 79: Preparation of (E)-N-(4-(1-(4-(4-(7-(2-(2,6-dioxopiperidin-3-yl)-1,3-dioxoisoindolin-4-yl)hept-6-yn-1-yl)piperazin-1-yl)benzoyl)piperidin-4-yl)butyl)-3-(pyridin-3-yl)acrylamide (SIAIS631108)

Referring to the methods of example 15 and Scheme 16, the compound (SIAIS631108) was prepared by using Daporinad derivative 1 (SIAIS630006) and intermediate LM (SIAIS292016; 7-(2-(2,6-dioxopiperidin-3-yl)-1,3-dioxoisoindolin-4-yl)hept-6-yn-1-yl methanesulfonate; CAS No.: 2641288-62-0). The compound (SIAIS631108) was obtained as white solid (7.7 mg, yield 23%). ¹H NMR (500 MHz, DMSO-d₆) δ 11.14 (s, 1H), 11.10 (s, 1H), 9.00 (d, J=2.1 Hz, 1H), 8.77 (d, J=5.3 Hz, 1H), 8.48 (d, J=8.1 Hz, 1H), 8.36 (t, J=5.6 Hz, 1H), 7.94-7.83 (m, 4H), 7.53 (d, J=15.9 Hz, 1H), 7.29 (d, J=8.5 Hz, 2H), 7.01 (d, J=8.7 Hz, 2H), 6.91 (d, J=15.9 Hz, 1H), 5.16 (dd, J=12.9, 5.4 Hz, 1H), 3.90 (d, J=13.0 Hz, 2H), 3.56 (d, J=11.8 Hz, 2H), 3.27-3.07 (m, 8H), 2.95-2.85 (m, 1H), 2.65-2.57 (m, 1H), 2.56 (t, J=6.9 Hz, 3H), 2.10-2.03 (m, 1H), 1.88-1.75 (m, 2H), 1.70-1.60 (m, 4H), 1.52-1.43 (m, 5H), 1.35-1.21 (m, 4H), 1.05 (m, 2H). HRMS (ESI) calcd for C₄₈H₅₆N₇O₆⁺ [M+H]⁺, 862.4287; found, 862.428.

### Example 80: Preparation of (E)-N-(4-(1-(4-(4-(8-(2-(2,6-dioxopiperidin-3-yl)-1,3-dioxoisoindolin-4-yl)oct-7-yn-1-yl)piperazin-1-yl)benzoyl)piperidin-4-yl)butyl)-3-(pyridin-3-yl)acrylamide (SIAIS631121)

Referring to the methods of example 15 and Scheme 16, the compound (SIAIS631121) was prepared by using Daporinad derivative 1 (SIAIS630006) and intermediate LM (SIAIS292008; 8-(2-(2,6-dioxopiperidin-3-yl)-1,3-dioxoisoindolin-4-yl)oct-7-yn-1-yl methanesulfonate; CAS No.: 2641288-63-1). The compound (SIAIS631121) was obtained as white solid (7.1 mg, yield 23%). ¹H NMR (500 MHz, CD₃OD) δ 8.70 (d, J=2.1 Hz, 1H), 8.49 (d, J=3.7 Hz, 1H), 8.16 (dt, J=8.1, 1.9 Hz, 1H), 7.78-7.66 (m, 3H), 7.54 (dd, J=8.1, 5.0 Hz, 1H), 7.47 (d, J=15.8 Hz, 1H), 7.26 (d, J=8.7 Hz, 2H), 6.97 (d, J=8.6 Hz, 2H), 6.70 (d, J=15.9 Hz, 1H), 5.04 (dd, J=12.8, 5.5 Hz, 1H), 4.46 (s, 1H), 3.86 (s, 2H), 3.72 (br, 1H), 3.58 (s, 2H), 3.23 (d, J=7.3 Hz, 2H), 3.16-3.09 (m, 4H), 3.05 (br, 2H), 2.82-2.73 (m, 2H), 2.69-2.56 (m, 2H), 2.44 (t, J=6.9 Hz, 2H), 2.08-2.00 (m, 1H), 1.82-1.69 (m, 3H), 1.60 (p, J=6.9 Hz, 3H), 1.55-1.45 (m, 6H), 1.41 (q, J=7.6 Hz, 2H), 1.37-1.29 (m, 2H), 1.28-1.18 (m, 2H), 1.06 (br, 2H). HRMS (ESI) calcd for C₄₉H₅₈N₇O₆⁺ [M+H]⁺, 840.4443; found, 840.4447.

### Example 81: Preparation of (E)-N-(4-(1-(4-(4-(2-(2-((2-(2,6-dioxopiperidin-3-yl)-1,3-dioxoisoindolin-4-yl)thio)ethoxy)acetyl)piperazin-1-yl)benzoyl)piperidin-4-yl)butyl)-3-(pyridin-3-yl)acrylamide (SIAIS631111)

Referring to the methods of example 1 and Scheme 15, the compound (SIAIS631111) was prepared by using Daporinad derivative 1 (SIAIS630006) and intermediate LM (SIAIS1204137; 2-(2-((2-(2,6-dioxopiperidin-3-yl)-1,3-dioxoisoindolin-4-yl)thio)ethoxy)acetic acid; CAS No.: 2378582-16-0). The compound (SIAIS631111) was obtained as yellow solid (8.0 mg, yield 24%). ¹H NMR (500 MHz, CD₃OD) δ 8.98 (s, 1H), 8.74 (d, J=5.6 Hz, 1H), 8.65 (d, J=8.4 Hz, 1H), 7.96 (dd, J=8.2, 5.5 Hz, 1H), 7.77 (d, J=8.0 Hz, 1H), 7.71 (t, J=7.8 Hz, 1H), 7.64-7.57 (m, 2H), 7.31 (d, J=8.8 Hz, 2H), 6.98 (d, J=8.9 Hz, 2H), 6.91 (d, J=15.9 Hz, 1H), 5.08 (dd, J=12.8, 5.5 Hz, 1H), 4.29 (s, 2H), 3.86 (t, J=6.0 Hz, 3H), 3.71-3.64 (m, 4H), 3.42-3.33 (m, 6H), 3.27-3.23 (m, 4H), 3.07 (br, 1H), 2.87-2.83 (m, 1H), 2.76-2.73 (m, 1H), 2.71-2.64 (m, 1H), 2.12-2.06 (m, 1H), 1.76 (br, 3H), 1.59 (p, J=7.2 Hz, 3H), 1.46-1.39 (m, 2H), 1.36-1.32 (m, 2H), 1.16 (s, 2H). HRMS (ESI) calcd for C₄₅H₅₂N₇O₈S⁺ [M+H]⁺, 850.3593; found, 850.3595.

### Example 82: Preparation of (E)-N-(4-(1-(4-(4-(2-(2-(2-((2-(2,6-dioxopiperidin-3-yl)-1,3-dioxoisoindolin-4-yl)thio)ethoxy)ethoxy)acetyl)piperazin-1-yl)benzoyl)piperidin-4-yl)butyl)-3-(pyridin-3-yl)acrylamide (SIAIS631112)

Referring to the methods of example 1 and Scheme 15, the compound (SIAIS631112) was prepared by using Daporinad derivative 1 (SIAIS630006) and intermediate LM (SIAIS1204139; 2-(2-(2-((2-(2,6-dioxopiperidin-3-yl)-1,3-dioxoisoindolin-4-yl)thio)ethoxy)ethoxy)acetic acid; CAS No.: 2378582-17-1). The compound (SIAIS631112) was obtained as yellow solid (8.7 mg, yield 23%). ¹H NMR (500 MHz, CD₃OD) δ 8.89 (d, J=19.8 Hz, 1H), 8.67 (dd, J=16.1, 5.2 Hz, 1H), 8.45 (dd, J=47.0, 8.4 Hz, 1H), 7.84-7.66 (m, 3H), 7.64-7.54 (m, 2H), 7.35 (d, J=8.8 Hz, 1H), 7.26 (d, J=8.7 Hz, 1H), 7.07 (d, J=8.9 Hz, 1H), 6.94 (d, J=8.8 Hz, 1H), 6.83 (dd, J=15.8, 10.7 Hz, 1H), 5.11 (dd, J=12.4, 5.5 Hz, 1H), 4.30 (s, 1H), 4.10 (s, 1H), 3.83-3.79 (m, 4H), 3.69-1.67 (m, 8H), 3.54-3.47 (m, 2H), 3.39-3.33 (m, 8H), 2.89-2.83 (m, 1H), 2.80-2.76 (m, 1H), 2.73-2.64 (m, 1H), 2.15-2.10 (m, 1H), 1.74 (br, 2H), 1.60-1.56 (m, 3H), 1.42 (br, 2H), 1.38-1.30 (m, 2H), 1.15 (br, 2H). HRMS (ESI) calcd for C₄₇H₅₆N₇O₉S⁺ [M+H]⁺, 894.3855; found, 894.3857.

### Example 83: Preparation of (E)-N-(4-(1-(4-(4-(2-(2-(2-(2-((2-(2,6-dioxopiperidin-3-yl)-1,3-dioxoisoindolin-4-yl)thio)ethoxy)ethoxy)ethoxy)acetyl)piperazin-1-yl)benzoyl)piperidin-4-yl)butyl)-3-(pyridin-3-yl)acrylamide (SIAIS631113)

Referring to the methods of example 1 and Scheme 15, the compound (SIAIS631113) was prepared by using Daporinad derivative 1 (SIAIS630006) and intermediate LM (SIAIS1204141; 2-(2-(2-(2-((2-(2,6-dioxopiperidin-3-yl)-1,3-dioxoisoindolin-4-yl)thio)ethoxy)ethoxy)ethoxy)acetic acid; CAS No.: 2378582-18-2). The compound (SIAIS631113) was obtained as yellow solid (8.4 mg, yield 22%). ¹H NMR (500 MHz, CD₃OD) δ 8.81 (s, 1H), 8.58 (d, J=5.4 Hz, 1H), 8.40 (dd, J=8.2, 1.8 Hz, 1H), 7.74 (dd, J=8.2, 5.4 Hz, 1H), 7.67-7.55 (m, 2H), 7.53-7.43 (m, 2H), 7.25 (d, J=8.8 Hz, 1H), 7.18 (d, J=8.8 Hz, 1H), 6.85 (d, J=8.9 Hz, 2H), 6.76 (d, J=15.8 Hz, 1H), 5.00 (dd, J=12.8, 5.5 Hz, 1H), 4.19 (s, 2H), 3.74-3.66 (m, 2H), 3.63-3.50 (m, 12H), 3.43-3.38 (m, 1H), 3.29-3.26 (m, 1H), 3.26-3.21 (m, 5H), 3.21-3.10 (m, 5H), 2.82-2.70 (m, 1H), 2.69-2.63 (m, 1H), 2.63-2.54 (m, 1H), 2.09-1.95 (m, 1H), 1.64 (br, 2H), 1.52-1.44 (m, 3H), 1.34-1.28 (m, 2H), 1.24-1.20 (m, 3H), 1.04 (br, 2H). HRMS (ESI) calcd for C₄₉H₆₀N₇O₁₀S⁺ [M+H]⁺, 938.4117; found, 938.4118.

### Example 84: Preparation of (E)-N-(4-(1-(4-(4-(14-((2-(2,6-dioxopiperidin-3-yl)-1,3-dioxoisoindolin-4-yl)thio)-3,6,9,12-tetraoxatetradecanoyl)piperazin-1-yl)benzoyl)piperidin-4-yl)butyl)-3-(pyridin-3-yl)acrylamide (SIAIS631114)

Referring to the methods of example 1 and Scheme 15, the compound (SIAIS631114) was prepared by using Daporinad derivative 1 (SIAIS630006) and intermediate LM (SIAIS1204147; 14-((2-(2,6-dioxopiperidin-3-yl)-1,3-dioxoisoindolin-4-yl)thio)-3,6,9,12-tetraoxatetradecanoic acid; CAS No.: 2378582-19-3). The compound (SIAIS631114) was obtained as yellow solid (11.3 mg, yield 29%). ¹H NMR (500 MHz, CD₃OD) δ 9.05 (s, 1H), 8.79 (d, J=6.4 Hz, 2H), 8.14-8.01 (m, 1H), 7.71 (q, J=7.8 Hz, 2H), 7.64-7.55 (m, 2H), 7.33 (d, J=8.5 Hz, 2H), 7.05 (d, J=8.5 Hz, 2H), 6.96 (d, J=15.8 Hz, 1H), 5.09 (dd, J=12.9, 5.5 Hz, 1H), 4.53 (br, 1H), 4.10 (s, 2H), 3.79 (t, J=6.2 Hz, 2H), 3.70-3.59 (m, 11H), 3.49 (dd, J=6.8, 3.8 Hz, 4H), 3.39-3.24 (m, 11H), 3.07 (br, 1H), 2.90-2.80 (m, 1H), 2.76-2.64 (m, 2H), 2.15-2.06 (m, 1H), 1.75 (d, J=45.8 Hz, 2H), 1.57 (p, J=7.4 Hz, 3H), 1.48-1.36 (m, 2H), 1.35-1.26 (m, 2H), 1.13 (br, 2H). HRMS (ESI) calcd for C₅₁H₆₄N₇O₁₁S⁺ [M+H]⁺, 982.4379; found, 982.4381.

### Example 85: Preparation of (E)-N-(4-(1-(4-(4-(17-((2-(2,6-dioxopiperidin-3-yl)-1,3-dioxoisoindolin-4-yl)thio)-3,6,9,12,15-pentaoxaheptadecanoyl)piperazin-1-yl)benzoyl)piperidin-4-yl)butyl)-3-(pyridin-3-yl)acrylamide (SIAIS631115)

Referring to the methods of example 1 and Scheme 15, the compound (SIAIS632027) was prepared by using Daporinad derivative 1 (SIAIS630006) and intermediate LM (SIAIS1204149; 17-((2-(2,6-dioxopiperidin-3-yl)-1,3-dioxoisoindolin-4-yl)thio)-3,6,9,12,15-pentaoxaheptadecanoic acid; CAS No.: 2378582-20-6). The compound (SIAIS632027) was obtained as yellow solid (12.4 mg, yield 30%). ¹H NMR (500 MHz, CD₃OD) δ 8.90 (s, 1H), 8.68 (d, J=5.4 Hz, 1H), 8.50-8.45 (m, 1H), 7.86-7.80 (m, 1H), 7.77-7.69 (m, 2H), 7.61-7.57 (m, 2H), 7.33 (dd, J=23.1, 8.7 Hz, 2H), 7.07 (d, J=8.5 Hz, 1H), 7.00-6.95 (m, 1H), 6.86 (dt, J=15.8, 1.9 Hz, 1H), 5.11 (dd, J=12.7, 6.1 Hz, 1H), 4.31 (s, 1H), 4.12 (s, 1H), 3.80 (q, J=6.4 Hz, 2H), 3.73-3.59 (m, 18H), 3.54-3.48 (m, 2H), 3.40-3.23 (m, 12H), 3.08 (s, 1H), 2.93-2.82 (m, 1H), 2.79-2.62 (m, 1H), 2.17-2.08 (m, 1H), 1.74 (br, 1H), 1.64-1.55 (m, 3H), 1.45-1.35 (m, 2H), 1.35-1.25 (m, 2H), 1.15 (br, 2H). HRMS (ESI) calcd for C₅₃H₆₈N₇O₁₂S⁺ [M+H]⁺, 1026.4641; found, 1026.4646.

### Example 86: Preparation of (E)-N-(4-(1-(4-(4-(17-((2-(2,6-dioxopiperidin-3-yl)-1-oxoisoindolin-4-yl)thio)-3,6,9,12,15-pentaoxaheptadecanoyl)piperazin-1-yl)benzoyl)piperidin-4-yl)butyl)-3-(pyridin-3-yl)acrylamide (SIAIS631118)

Referring to the methods of example 1 and Scheme 15, the compound (SIAIS631118) was prepared by using Daporinad derivative 1 (SIAIS630006) and intermediate LM (SIAIS1213137; 17-((2-(2,6-dioxopiperidin-3-yl)-1-oxoisoindolin-4-yl)thio)-3,6,9,12,15-pentaoxaheptadecanoic acid; CAS No.: 2378582-25-1). The compound (SIAIS631118) was obtained as white solid (13.1 mg, yield 32%). ¹H NMR (500 MHz, CD₃OD) δ 9.03 (d, J=2.1 Hz, 1H), 8.78 (d, J=5.5 Hz, 1H), 8.74 (dt, J=8.2, 1.7 Hz, 1H), 8.04 (dd, J=8.2, 5.6 Hz, 1H), 7.71 (dd, J=7.8, 0.9 Hz, 1H), 7.69-7.59 (m, 2H), 7.54 (t, J=7.6 Hz, 1H), 7.36 (d, J=8.8 Hz, 2H), 7.07 (d, J=8.8 Hz, 2H), 6.94 (d, J=15.9 Hz, 1H), 5.16 (dd, J=13.4, 5.3 Hz, 1H), 4.49 (d, J=17.3 Hz, 1H), 4.43 (d, J=17.4 Hz, 1H), 4.12 (s, 2H), 3.71-3.63 (m, 7H), 3.62-3.57 (m, 14H), 3.51 (dd, J=6.7, 3.8 Hz, 3H), 3.38 (dd, J=6.6, 3.9 Hz, 3H), 3.35-3.30 (m, 4H), 3.25-3.22 (m, 3H), 2.96-2.87 (m, 1H), 2.82-2.75 (m, 1H), 2.58-2.50 (m, 1H), 2.22-2.17 (m, 1H), 1.73 (br, 2H), 1.62-1.56 (m, 3H), 1.48-1.39 (m, 2H), 1.36-1.32 (m, 2H), 1.16 (br, 2H). HRMS (ESI) calcd for C₅₃H₇₀N₇₀O₁₁S⁺ [M+H]⁺, 1012.4849; found, 1012.4851.

### Example 87: Preparation of (E)-N-(4-(1-(4-(4-(6-((2-(2,6-dioxopiperidin-3-yl)-1-oxoisoindolin-4-yl)oxy)hexyl)piperazin-1-yl)benzoyl)piperidin-4-yl)butyl)-3-(pyridin-3-yl)acrylamide (SIAIS633097)

Referring to the methods of example 15 and Scheme 16, the compound (SIAIS633097) was prepared by using Daporinad derivative 1 (SIAIS630006) and intermediate LM (3-(4-((6-bromohexyl)oxy)-1-oxoisoindolin-2-yl)piperidine-2,6-dione; CAS No.: 2413731-77-6). The compound (SIAIS633097) was obtained as white solid (5.7 mg, yield 17%). ¹H NMR (500 MHz, DMSO-d6) δ 10.98 (s, 1H), 8.80 (d, J=2.2 Hz, 1H), 8.59 (dd, J=4.9, 1.6 Hz, 1H), 8.36 (s, 1H), 8.09 (d, J=8.1 Hz, 1H), 7.53 (dd, J=8.0, 4.9 Hz, 1H), 7.50-7.44 (m, 2H), 7.39 (d, J=8.7 Hz, 2H), 7.31 (d, J=7.5 Hz, 1H), 7.26-7.22 (m, 1H), 7.04 (d, J=9.1 Hz, 2H), 6.76 (d, J=15.9 Hz, 1H), 5.10 (dd, J=13.3, 5.1 Hz, 1H), 4.37 (d, J=17.4 Hz, 1H), 4.22 (d, J=17.4 Hz, 1H), 4.13 (t, J=6.3 Hz, 2H), 3.93 (d, J=11.0 Hz, 2H), 3.36-3.23 (m, 5H), 3.22 (q, J=6.6 Hz, 4H), 3.18-3.07 (m, 12H), 2.94-2.80 (m, 1H), 2.65-2.55 (m, 1H), 2.46-2.42 (m, 1H), 2.00 (ddd, J=12.4, 6.4, 3.8 Hz, 1H), 1.79-1.61 (m, 6H), 1.49 (dt, J=14.1, 7.1 Hz, 4H), 1.38 (q, J=7.5 Hz, 2H). HRMS (ESI) calcd for C₄₇H₆₀N₇O₆⁺ [M+H]⁺, 818.4600; found, 818.4607.

### Example 88: Preparation of (E)-N-(4-(1-(4-(4-(7-((2-(2,6-dioxopiperidin-3-yl)-1-oxoisoindolin-4-yl)oxy)heptyl)piperazin-1-yl)benzoyl)piperidin-4-yl)butyl)-3-(pyridin-3-yl)acrylamide (SIAIS633098)

Referring to the methods of example 15 and Scheme 16, the compound (SIAIS633098) was prepared by using Daporinad derivative 1 (SIAIS630006) and intermediate LM (3-(4-((7-bromoheptyl)oxy)-1-oxoisoindolin-2-yl)piperidine-2,6-dione; CAS No.: 2699962-33-7). The compound (SIAIS633098) was obtained as white solid (6.3 mg, yield 19%). ¹H NMR (500 MHz, DMSO-d6) δ 10.98 (s, 1H), 8.86 (d, J=2.2 Hz, 1H), 8.64 (dd, J=5.0, 1.5 Hz, 1H), 8.42 (t, J=5.7 Hz, 1H), 8.21 (dt, J=8.1, 1.9 Hz, 1H), 7.63 (dd, J=8.0, 5.0 Hz, 1H), 7.52-7.46 (m, 2H), 7.39 (d, J=8.5 Hz, 2H), 7.32 (d, J=7.5 Hz, 1H), 7.24 (d, J=8.1 Hz, 1H), 7.05 (d, J=8.8 Hz, 2H), 6.82 (d, J=15.9 Hz, 1H), 5.10 (dd, J=13.3, 5.1 Hz, 1H), 4.38 (d, J=17.4 Hz, 1H), 4.23 (d, J=17.4 Hz, 1H), 4.13 (t, J=6.4 Hz, 2H), 3.93 (d, J=12.6 Hz, 2H), 3.27-3.03 (m, 19H), 2.98-2.81 (m, 1H), 2.66-2.57 (m, 1H), 2.45 (dd, J=13.2, 4.5 Hz, 1H), 2.01 (ddd, J=7.1, 5.3, 2.3 Hz, 1H), 1.78-1.70 (m, 8H), 1.50 (dq, J=30.1, 7.3 Hz, 4H), 1.41-1.30 (m, 4H). HRMS (ESI) calcd for C₄₈H₆₂N₇O₆⁺ [M+H]⁺, 832.4756; found, 832.4758.

### Example 89: Preparation of (E)-N-(4-(1-(6-(4-(4-(7-(2-(2,6-dioxopiperidin-3-yl)-1-oxoisoindolin-4-yl)hept-6-yn-1-yl)piperazin-1-yl)piperidin-1-yl)nicotinoyl)piperidin-4-yl)butyl)-3-(pyridin-3-yl)acrylamide (SIAIS633093)

Referring to the methods of example 15 and Scheme 16, the compound (SIAIS633093) was prepared by using Daporinad derivative 7 (SIAIS632044) and intermediate LM (SIAIS292017; 7-(2-(2,6-dioxopiperidin-3-yl)-1-oxoisoindolin-4-yl)hept-6-yn-1-yl methanesulfonate; CAS No.: 2570254-41-8). The compound (SIAIS633093) was obtained as white solid (5.8 mg, yield 18%). ¹H NMR (500 MHz, DMSO-d6) δ 10.99 (s, 1H), 8.88 (d, J=2.1 Hz, 1H), 8.65 (d, J=5.3 Hz, 1H), 8.32-8.25 (m, 1H), 8.14 (d, J=2.4 Hz, 1H), 7.73-7.64 (m, 2H), 7.64-7.57 (m, 1H), 7.52 (t, J=7.6 Hz, 1H), 7.48 (d, J=15.9 Hz, 1H), 6.97 (d, J=9.1 Hz, 1H), 6.80 (d, J=15.9 Hz, 1H), 5.13 (dd, J=13.3, 5.1 Hz, 1H), 4.51 (d, J=13.0 Hz, 2H), 4.45 (d, J=17.6 Hz, 1H), 4.31 (d, J=17.7 Hz, 1H), 3.79-3.65 (m, 11H), 3.23-3.06 (m, 5H), 2.90 (t, J=13.8 Hz, 4H), 2.61 (d, J=17.3 Hz, 1H), 2.47-2.35 (m, 1H), 2.18-2.11 (m, 1H), 2.09-1.98 (m, 1H), 1.74-1.53 (m, 8H), 1.44 (d, J=9.0 Hz, 5H), 1.40-1.27 (m, 4H), 1.28-1.19 (m, 3H), 1.09-1.03 (m, 2H). HRMS (ESI) calcd for C₅₂H₆₆N₉O₅⁺ [M+H]⁺, 896.5181; found, 896.5186.

### Example 90: Preparation of (E)-N-(4-(1-(6-(4-(4-(8-(2-(2,6-dioxopiperidin-3-yl)-1-oxoisoindolin-4-yl)oct-7-yn-1-yl)piperazin-1-yl)piperidin-1-yl)nicotinoyl)piperidin-4-yl)butyl)-3-(pyridin-3-yl)acrylamide (SIAIS633094)

Referring to the methods of example 15 and Scheme 16, the compound (SIAIS633094) was prepared by using Daporinad derivative 7 (SIAIS632044) and intermediate LM (SIAIS292020; 8-(2-(2,6-dioxopiperidin-3-yl)-1-oxoisoindolin-4-yl)oct-7-yn-1-yl methanesulfonate; CAS No.: 2570254-42-9). The compound (SIAIS633094) was obtained as white solid (6.1 mg, yield 19%). ¹H NMR (500 MHz, DMSO-d6) δ 10.99 (s, 1H), 8.95 (d, J=2.1 Hz, 1H), 8.72 (d, J=5.3 Hz, 1H), 8.42 (d, J=8.1 Hz, 1H), 8.34 (t, J=5.7 Hz, 1H), 8.13 (d, J=2.3 Hz, 1H), 7.81 (dd, J=8.1, 5.3 Hz, 1H), 7.71 (s, 1H), 7.67-7.62 (m, 2H), 7.51 (dd, J=16.0, 9.0 Hz, 2H), 7.03 (d, J=9.1 Hz, 1H), 6.85 (d, J=15.9 Hz, 1H), 5.13 (dd, J=13.3, 5.1 Hz, 1H), 4.51 (d, J=13.2 Hz, 2H), 4.46 (d, J=17.7 Hz, 1H), 4.32 (d, J=17.6 Hz, 1H), 3.85-3.66 (m, 12H), 3.20-3.15 (m, 4H), 3.04-2.84 (m, 4H), 2.72 (d, J=4.9 Hz, 1H), 2.64-2.53 (m, 1H), 2.59-2.50 (m, 4H), 2.49-2.38 (m, 1H), 2.18 (d, J=11.9 Hz, 2H), 2.06-1.95 (m, 1H), 1.81-1.56 (m, 7H), 1.45 (dt, J=15.2, 7.9 Hz, 4H), 1.36-1.17 (m, 5H), 1.07-1.03 (m, 2H). HRMS (ESI) calcd for C₅₃H₆₈N₉O₅⁺ [M+H]⁺, 910.5338; found, 910.5341.

### Example 91: Preparation of (2S,4R)-1-((S)-3,3-dimethyl-2-(6-oxo-6-(4-(1-(6-(4-(4-((E)-3-(pyridin-3-yl)acrylamido)butyl)piperidine-1-carbonyl)pyridazin-3-yl)piperidin-4-yl)piperazin-1-yl)hexanamido)butanoyl)-4-hydroxy-N-(4-(4-methylthiazol-5-yl)benzyl)pyrrolidine-2-carboxamide (SIAIS633063)

Referring to the methods of example 1 and Scheme 15, the compound (SIAIS633063) was prepared by using Daporinad derivative 5 (SIAIS631135) and intermediate LM (SIAIS074013; 6-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-6-oxohexanoic acid; CAS No.: 2172819-74-6). The compound (SIAIS633063) was obtained as white solid (9.8 mg, yield 25%). ¹H NMR (500 MHz, DMSO-d6) δ 9.00 (d, J=1.7 Hz, 2H), 8.76 (d, J=5.4 Hz, 1H), 8.60 (t, J=6.2 Hz, 1H), 8.52 (d, J=8.2 Hz, 1H), 8.38 (t, J=5.7 Hz, 1H), 7.95-7.85 (m, 2H), 7.71-7.60 (m, 1H), 7.58-7.47 (m, 2H), 7.44-7.32 (m, 4H), 6.89 (d, J=15.9 Hz, 1H), 4.61-4.50 (m, 3H), 4.48-4.36 (m, 4H), 4.35 (s, 1H), 4.21 (dd, J=16.0, 5.5 Hz, 1H), 4.04 (s, 2H), 3.94-3.85 (m, 3H), 3.78-3.72 (m, 4H), 3.19 (q, J=6.5 Hz, 2H), 3.03 (q, J=14.1, 13.1 Hz, 4H), 2.94-2.88 (m, 1H), 2.77 (t, J=12.3 Hz, 1H), 2.43 (s, 3H), 2.37-2.30 (m, 2H), 2.29-2.09 (m, 4H), 2.04 (t, J=9.1 Hz, 1H), 1.89 (ddd, J=12.9, 8.8, 4.6 Hz, 1H), 1.80-1.61 (m, 4H), 1.58-1.35 (m, 7H), 1.35-1.29 (m, 2H), 1.27-1.20 (m, 3H), 1.12-1.05 (m, 2H), 0.92 (s, 9H). HRMS (ESI) calcd for C₅₉H₈₁N₁₂O₇S⁺ [M+H]⁺, 1101.6066; found, 1101.6069.

### Biological Activity Assay

### Materials:

| Reagents and biological materials | SOURCE |
|---|---|
| colorectal cancer cells: SW620 | ATCC (American Type Culture |
| | Collection) |
| colorectal cancer cells: HT29 | ATCC |
| breast cancer cells: MCF-7 | ATCC |
| hepatoma cells: BEL7404 | ATCC |
| hematologic cancer cells: Jurkat | ATCC |
| hematologic cancer cells: HL60 | ATCC |
| hematologic cancer cells: MOLT4 | ATCC |
| RPMI1640 | Thermo Company |
| DMEM | Thermo Company |
| CellTiter-Glo ® Luminescent Cell | Promega Company |
| Viability Assay (CTG Reagent) | |
| Protease Inhibitor | Roche Company |
| Phosphatase Inhibitor | Yeasen Biotechnology |
| | (Shanghai) Co., Ltd. |
| Fetal bovine serum (FBS) | Sunrise Science Products Company |
| Penicillin-Streptomycin | Sunrise Science Products Company |
| SuperSignal West Pico PLUS | Thermo Fisher Company |
| Chemiluminescent Substrate | |
| anti-NAMPT Antibody | Proteintech Company, Cat No.: |
| | 66385-1-lg |
| anti-GAPDH Antibody | Abclonal Company, Cat No.: A19056 |
| Peroxidase-labeled Goat | Jackson ImmunoResearch |
| Anti-Rabbit IgG (H + L) | Laboratories Inc., Cat |
| | No.: 111-035-003 |

### Methods

### Cell Cultures

The tumor cells used herein were cultured in a cell culture medium at 37° C. in an incubator with 5% CO₂, where the cell culture medium was supplemented with 10% FBS, as well as penicillin at a final concentration of 100 U/mL and streptomycin at a final concentration of 100 μg/mL. Prior to experimentation, all cells used were identified as correct cells by STR cells, and were negative for mycoplasma through routine testing.

### Determination of Half Inhibitory Concentration (IC₅₀) of the Compounds:

IC₅₀ values of the compounds of the present disclosure (including the compounds in Table 1 and compounds of examples) were measured using the CellTiter-Glo® Luminescent Cell Viability Assay kit from Promega Corporation. Assay details are as follows: Cells were seeded in 100 μL RPMI1640 medium containing serum at a density of 20,000 cells/well. After 24 h, the inoculated cells were treated with the compounds of the present disclosure which were serially diluted. After the cells were treated with the compounds of the present disclosure for 72 h, the CellTiter-Glo® reagent was added to the culture medium for cell viability assessment according to the instructions provided with the kit. DMSO served as the negative control, and FK866 inhibitor was used as the positive control, both treated with the same protocol as the compounds of the present disclosure. The growth inhibition of the compounds of the present disclosure on cells was plotted by Prism Graphpad software, and the IC₅₀ values of the compounds of the present disclosure were calculated therefrom. Results were shown in Table 2.

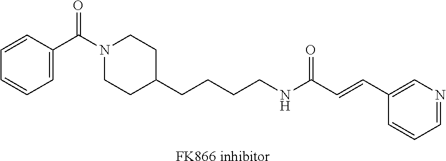 [SMILES: O=C(/C=C/c1cccnc1)NCCCCC1CCN(C(=O)c2ccccc2)CC1 ; "FK866 inhibitor"]

TABLE 2
The IC₅₀ values of the compounds of the present disclosure
on the proliferation inhibitory activity of tumor cells

| | Jurkat/ | | Jurkat/ | | Jurkat/ |
|---|---|---|---|---|---|
| Compounds | IC₅₀(nM) | Compounds | IC₅₀(nM) | Compounds | IC₅₀(nM) |
| FK866 inhibitor | 0.6350 | SIAIS632031 | 24.36 | SIAIS630119 | 2.989 |
| SIAIS630010 | 0.3506 | SIAIS630038 | 24.67 | SIAIS630131 | 4.381 |
| SIAIS632041 | 1.032 | SIAIS632029 | 28.73 | SIAIS632011 | 9.303 |
| SIAIS630037 | 1.14 | SIAIS632010 | 8.347 | SIAIS630122 | 28.27 |
| SIAIS632012 | 1.655 | SIAIS632036 | 29.28 | SIAIS631045 | 79.95 |
| SIAIS630014 | 1.988 | SIAIS632018 | 55.52 | SIAIS630130 | 24.2 |
| SIAIS630009 | 2.29 | SIAIS631078 | 62.23 | SIAIS630013 | 190.3 |
| SIAIS630123 | 2.518 | SIAIS631073 | 62.45 | SIAIS632034 | 95.11 |
| SIAIS632039 | 2.753 | SIAIS631070 | 62.91 | SIAIS631071 | 634.6 |
| SIAIS630012 | 2.824 | SIAIS631072 | 63.05 | SIAIS632015 | 523.4 |
| SIAIS630011 | 2.848 | SIAIS631121 | 63.94 | SIAIS632004 | 361.8 |
| SIAIS630129 | 2.932 | SIAIS631111 | 64.29 | SIAIS631135 | 724.8 |
| SIAIS630006 | 3.004 | SIAIS632016 | 64.37 | SIAIS630020 | 849.2 |
| SIAIS632007 | 3.588 | SIAIS630136 | 64.51 | SIAIS631079 | 7.319 |
| SIAIS632005 | 3.98 | SIAIS631114 | 66.52 | SIAIS630039 | 7.955 |
| SIAIS630135 | 4.02 | SIAIS631118 | 66.62 | SIAIS632035 | 8.524 |
| SIAIS630121 | 4.825 | SIAIS631112 | 66.68 | SIAIS631140 | 9.704 |
| SIAIS631139 | 5.232 | SIAIS631113 | 68.11 | SIAIS632030 | 11.03 |
| SIAIS631141 | 5.363 | SIAIS631120 | 70.97 | SIAIS632017 | 21.18 |
| SIAIS632040 | 5.675 | SIAIS631069 | 71.16 | SIAIS632028 | 105.4 |
| SIAIS630120 | 6.235 | SIAIS631109 | 71.22 | SIAIS630007 | 109.4 |
| SIAIS630132 | 6.295 | SIAIS631115 | 74.21 | SIAIS631011 | 139.5 |
| SIAIS630133 | 6.366 | SIAIS631068 | 74.42 | SIAIS632032 | 191.5 |
| SIAIS632006 | 6.395 | SIAIS632033 | 78.73 | SIAIS632027 | 294.5 |
| SIAIS632014 | 6.806 | SIAIS631108 | 79.71 | SIAIS631119 | 622.8 |
| SIAIS630134 | 7.282 | SIAIS630008 | 96.18 | SIAIS633097 | 78.96 |
| SIAIS633098 | 33.9 | SIAIS633093 | 15.79 | SIAIS633094 | 6.183 |
| SIAIS633063 | 255.4 | | | | |

The results indicate that the compounds of the present invention (including the compounds in Table 1 and compounds of the examples 1-91) can inhibit the proliferation of tumor cells (as shown in Table 2). The IC₅₀ of some example compounds are comparable to, or even superior to, the positive control FK866. For example, the IC₅₀ of SIAIS630010 is 0.3506 nM. This indicates that the compounds of the present invention have a comparable, or even superior, inhibitory effect on tumor cell growth compared to the positive control FK866.

### In Vitro Cytotoxicity Assay of the Compounds of the Present Disclosure Against Tumor Cells

MOLT4 and HL60 tumor cells were seeded separately in 12-well plates containing 1 mL of RPMI1640 medium, with a cell seeding density of 0.5×10⁶ cells/mL. On the second day, the parent inhibitor FK866 and the compounds SIAIS630120 and SIAIS630121 of the present disclosure were individually dissolved in DMSO to obtain stock solutions of the respective compounds at a concentration of 10 μM. Subsequently, 1 μL of the 10 μM compound stock solution was added to 1 mL of cell culture medium to achieve a final concentration of 10 nM. The cells were treated continuously for 4 days, and cell viability was measured once daily during this period. Specifically, cells were sampled once a day, adjusted to a concentration of 1×10⁶/mL using a cell staining buffer, stained with 4′,6-diamidino-2-phenylindole dihydrochloride (DAPI) (1:250), and then analyzed with a flow cytometer. Initially, the cells were gated based on differences in cell size, followed by DAPI staining to gate for dead cells. Finally, the cytotoxic efficiency was assessed by calculating the DAPI-positive ratio.

The results of the in vitro cytotoxicity experiment were shown in FIGS. 6-7. The results indicated that for HL60 cells, both PROTAC compounds, SIAIS630120 and SIAIS630121, achieved a cytotoxic effect of 95% on the 4^{th} day, while the FK866-treated group showed less than 40% cell death. Regarding MOLT4 cells, these two PROTAC compounds exhibited approximately 50% cytotoxicity on the 4^{th} day, possibly due to higher tolerance of MOLT4 cells to the two PROTAC compounds, but still 20% more effective than FK866. Thus, the cytotoxic effects of the PROTAC compounds of the present invention are significantly superior to FK866.

### Determination of Half-Maximal Target Protein Degradation Concentration (DC₅₀) of the Compounds of the Present Disclosure to Target Proteins

### Western Blot

Tumor cells were seeded at a specific cell density in a 6-well plate containing 2 mL of RPMI1640/DMEM medium. For SW620, HT29, MCF-7, and BEL7404 cells, the seeding density was 1×10⁶ cells per well, respectively; for MOLT4, Jurkat, and HL60 cells, the seeding density was 0.5×10⁶ cells/mL. The disclosed compounds (including those listed in Table 1 and Examples 1-91) were individually dissolved in DMSO to prepare stock solutions at concentrations of 1 μM, 5 μM, 10 μM, and 100 μM. On the second day, cells were treated with different concentrations of the compounds of the present disclosure (including those listed in Table 1 and Examples 1-91) for 24 hours (2 μL of different concentrations of compounds added to 2 mL of cell culture medium, resulting in final concentrations of 1 nM, 5 nM, 10 nM, and 100 nM). After 24 hours of compound treatment, the supernatant was removed, and cells were washed with PBS. The cells were then placed on ice and treated with protein lysis buffer containing protease and phosphatase inhibitors. The lysate was centrifuged at 12,000 RPM for 15 minutes at 4° C., and the supernatant was collected. Equal amounts of protein were mixed with 5×SDS, and after denaturation at 95° C. for 5 minutes, the samples were either frozen at −20° C. or subjected to direct protein electrophoresis. SDS-PAGE was performed using equal amounts of protein, and proteins were transferred to a nitrocellulose membrane at 95 V and 4° C. for 90 minutes. After membrane transfer, the membrane was blocked with TBST containing 5% skim milk powder (purchased from Beijing Cowin Bioscience Co., Ltd.) at room temperature for 1 hour. The membrane was then incubated with the primary antibody overnight at 4° C. After incubation with the primary antibody, the membrane was washed three times with TBST at room temperature for 5 minutes each. Subsequent steps, including the incubation with the secondary antibody (peroxidase-labeled goat anti-rabbit IgG (H+L)) and visualization, were performed according to the instructions provided by Jackson ImmunoResearch Laboratories Inc.

Determining Half-Maximal Degradation Concentration (DC₅₀) value (the drug concentration required for degrading proteins by 50%, abbreviated as DC₅₀) reads method: comparing the grayscale values of the Western blotting bands for the drug treatment with those of the Western blotting band f after treatment with blank DMSO. The DC₅₀ is identified within the range of drug concentrations where the grayscale value matches half of the corresponding Western blotting band's grayscale value after treatment with blank DMSO.

Calculation of the DC₅₀ values can be performed using ImageJ to read the grayscale values of Western blotting bands after drug treatment. A curve fitting the relationship between drug concentration and grayscale values is then generated to estimate the drug concentration corresponding to half of the grayscale value.

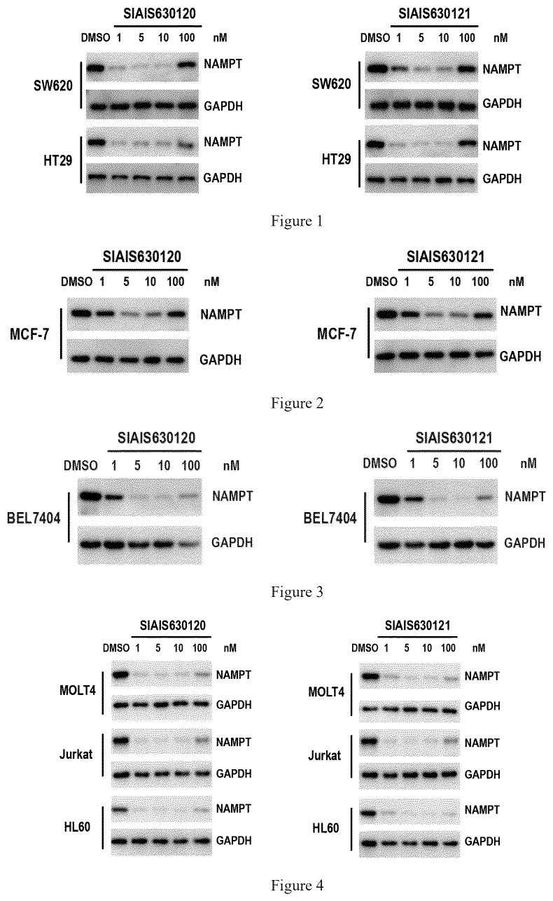

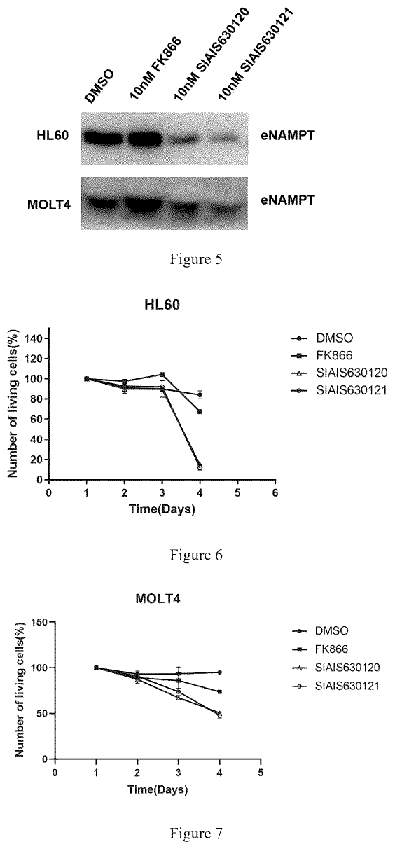

The present disclosure employed Western blot analysis to assess the expression levels of NAMPT protein in tumor cells treated with the compounds of the present disclosure (including those listed in Table 1 and Examples 1-91) for 24 hours. The Western blot results are illustrated in FIGS. 1-5. The experimental results demonstrated that the compounds of the present disclosure (including those listed in Table 1 and Examples 1-91) can promote the degradation of intracellular NAMPT (iNAMPT) and extracellular NAMPT (eNAMPT) proteins. Both iNAMPT and eNAMPT contribute to tumor growth. However, the commercially available parent inhibitor FK866 only inhibits the activity of iNAMPT protein and cannot degrade iNAMPT and eNAMPT proteins simultaneously, as the degrader compounds of the present invention can. Solely inhibiting iNAMPT protein activity triggers feedback regulation in cells, leading to the generation of more iNAMPT protein and promoting the secretion process to produce more, leading to the production of more iNAMPT protein and promoting the secretion process to produce more eNAMPT, significantly impacting the therapeutic effectiveness. This aspect has been confirmed by the Western blot results shown in FIG. 5. In FIG. 5, compared to the DMSO control group, treatment of MOLT4 cells with 10 nM FK866 inhibitor resulted in higher levels of eNAMPT protein.

The basic principles, main features and advantages of the present disclosure are shown and described above. Those skilled in the art should understand that the present disclosure is not limited by the foregoing embodiments, and they can make various changes, substitutions and alterations herein without departing from the spirit and scope of the present disclosure. These changes, substitutions and alterations fall within the scope of the present disclosure. The claimed scope of the present disclosure is defined by the appended claims and their equivalents.

### REFERENCES

1. Diefenbach, J. & Bürkle, A. Introduction to poly(ADP-ribose) metabolism. Cell Mol Life Sci 62, 721-730, doi:10.1007/s00018-004-4503-3 (2005).
2. Rajman, L., Chwalek, K. & Sinclair, D. A. Therapeutic Potential of NAD-Boosting Molecules: The In Vivo Evidence. Cell Metab 27, 529-547, doi:10.1016/j.cmet.2018.02.011 (2018).
3. Navas, L. E. & Carnero, A. NAD(+) metabolism, stemness, the immune response, and cancer. Signal Transduct Target Ther 6, 2, doi:10.1038/s41392-020-00354-w (2021).
4. Katsyuba, E. et al. De novo NAD(+) synthesis enhances mitochondrial function and improves health. Nature 563, 354-359, doi:10.1038/s41586-018-0645-6 (2018).
5. Badawy, A. A. Kynurenine Pathway of Tryptophan Metabolism: Regulatory and Functional Aspects. Int J Tryptophan Res 10, 1178646917691938, doi:10.1177/1178646917691938 (2017).
6. Youn, H. S. et al. Structural Insights into the Quaternary Catalytic Mechanism of Hexameric Human Quinolinate Phosphoribosyltransferase, a Key Enzyme in de novo NAD Biosynthesis. Sci Rep 6, 19681, doi:10.1038/srep19681 (2016).
7. Marletta, A. S. et al. Crystal structure of human nicotinic acid phosphoribosyltransferase. FEBS Open Bio 5, 419-428, doi:10.1016/j.fob.2015.05.002 (2015).
8. Brazill, J. M., Li, C., Zhu, Y. & Zhai, R. G. NMNAT: It's an NAD(+) synthase . . . . It's a chaperone . . . . It's a neuroprotector. Curr Opin Genet Dev 44, 156-162, doi:10.1016/j.gde.2017.03.014 (2017).
9. Rizzi, M., Bolognesi, M. & Coda, A. A novel deamido-NAD+-binding site revealed by the trapped NAD-adenylate intermediate in the NAD+ synthetase structure. Structure 6, 1129-1140, doi:10.1016/s0969-2126(98)00114-2 (1998).
10. Wang, T. et al. Structure of Nampt/PBEF/visfatin, a mammalian NAD+ biosynthetic enzyme. Nat Struct Mol Biol 13, 661-662, doi:10.1038/nsmb1114 (2006).
11. Chiarugi, A., Dölle, C., Felici, R. & Ziegler, M. The NAD metabolome—a key determinant of cancer cell biology. Nat Rev Cancer 12, 741-752, doi:10.1038/nrc3340 (2012).
12. Sampath, D., Zabka, T. S., Misner, D. L., O'Brien, T. & Dragovich, P. S. Inhibition of nicotinamide phosphoribosyltransferase (NAMPT) as a therapeutic strategy in cancer. Pharmacol Ther 151, 16-31, doi:10.1016/j.pharmthera.2015.02.004 (2015).
13. Chowdhry, S. et al. NAD metabolic dependency in cancer is shaped by gene amplification and enhancer remodelling. Nature 569, 570-575, doi:10.1038/s41586-019-1150-2 (2019).
14. Xiao, Y. et al. Dependence of tumor cell lines and patient-derived tumors on the NAD salvage pathway renders them sensitive to NAMPT inhibition with GNE-618. Neoplasia 15, 1151-1160, doi:10.1593/neo.131304 (2013).
15. Shackelford, R. E., Mayhall, K., Maxwell, N. M., Kandil, E. & Coppola, D. Nicotinamide phosphoribosyltransferase in malignancy: a review. Genes Cancer 4, 447-456, doi:10.1177/1947601913507576 (2013).
16. Li, X. Q. et al. NAMPT and NAPRT, Key Enzymes in NAD Salvage Synthesis Pathway, Are of Negative Prognostic Value in Colorectal Cancer. Front Oncol 9, 736, doi:10.3389/fonc.2019.00736 (2019).
17. Zhang, H. et al. Epigenetic Regulation of NAMPT by NAMPT-AS Drives Metastatic Progression in Triple-Negative Breast Cancer. Cancer Res 79, 3347-3359, doi:10.1158/0008-5472.Can-18-3418 (2019).
18. Meram, A. T. et al. Nicotinamide Phosphoribosyl Transferase Is Increased in Osteosarcomas and Chondrosarcomas Compared to Benign Bone and Cartilage. Anticancer Res 39, 1761-1765, doi:10.21873/anticanres.13282 (2019).
19. Davis, K. et al. Nicotinamide phosphoribosyltransferase expression and clinical outcome of resected stage I/II pancreatic ductal adenocarcinoma. PLoS One 14, e0213576, doi:10.1371/journal.pone.0213576 (2019).
20. Zhou, S. J., Bi, T. Q., Qin, C. X., Yang, X. Q. & Pang, K. Expression of NAMPT is associated with breast invasive ductal carcinoma development and prognosis. Oncol Lett 15, 6648-6654, doi:10.3892/ol.2018.8164 (2018).
21. Wang, B. et al. NAMPT overexpression in prostate cancer and its contribution to tumor cell survival and stress response. Oncogene 30, 907-921, doi:10.1038/onc.2010.468 (2011).
22. Wang, X. Y. et al. Inhibition of nicotinamide phosphoribosyltransferase and depletion of nicotinamide adenine dinucleotide contribute to arsenic trioxide suppression of oral squamous cell carcinoma. Toxicol Appl Pharmacol 331, 54-61, doi:10.1016/j.taap.2017.05.008 (2017).
23. Vora, M. et al. Increased Nicotinamide Phosphoribosyltransferase in Rhabdomyosarcomas and Leiomyosarcomas Compared to Skeletal and Smooth Muscle Tissue. Anticancer Res 36, 503-507 (2016).
24. Li, H. et al. Role of Nampt and Visceral Adiposity in Esophagogastric Junction Adenocarcinoma. J Immunol Res 2017, 3970605, doi:10.1155/2017/3970605 (2017).
25. Zhu, Y. et al. Biomarker triplet NAMPT/VEGF/HER2 as a de novo detection panel for the diagnosis and prognosis of human breast cancer. Oncol Rep 35, 454-462, doi:10.3892/or.2015.4391 (2016).
26. Lv, X. et al. Regulative Effect of Nampt on Tumor Progression and Cell Viability in Human Colorectal Cancer. J Cancer 6, 849-858, doi:10.7150/jca.12341 (2015).
27. Sawicka-Gutaj, N. et al. Nicotinamide phosphorybosiltransferase overexpression in thyroid malignancies and its correlation with tumor stage and with survivin/survivin DEx3 expression. Tumour Biol 36, 7859-7863, doi:10.1007/s13277-015-3506-z (2015).
28. Audrito, V. et al. Extracellular nicotinamide phosphoribosyltransferase (NAMPT) promotes M2 macrophage polarization in chronic lymphocytic leukemia. Blood 125, 111-123, doi:10.1182/blood-2014-07-589069 (2015).
29. Shackelford, R. et al. Nicotinamide phosphoribosyltransferase and SIRT3 expression are increased in well-differentiated thyroid carcinomas. Anticancer Res 33, 3047-3052 (2013).
30. Olesen, U. H., Hastrup, N. & Sehested, M. Expression patterns of nicotinamide phosphoribosyltransferase and nicotinic acid phosphoribosyltransferase in human malignant lymphomas. Apmis 119, 296-303, doi:10.1111/j.1600-0463.2011.02733.x (2011).
31. Shackelford, R. E., Bui, M. M., Coppola, D. & Hakam, A. Over-expression of nicotinamide phosphoribosyltransferase in ovarian cancers. Int J Clin Exp Pathol 3, 522-527 (2010).
32. Shackelford, R. E. et al. Increased Nicotinamide Phosphoribosyltransferase and Cystathionine-β-Synthase in Renal Oncocytomas, Renal Urothelial Carcinoma, and Renal Clear Cell Carcinoma. Anticancer Res 37, 3423-3427, doi:10.21873/anticanres.11709 (2017).
33. Hasmann, M. & Schemainda, I. FK866, a highly specific noncompetitive inhibitor of nicotinamide phosphoribosyltransferase, represents a novel mechanism for induction of tumor cell apoptosis. Cancer Res 63, 7436-7442 (2003).
34. Clark, J. B., Ferris, G. M. & Pinder, S. Inhibition of nuclear NAD nucleotidase and poly ADP-ribose polymerase activity from rat liver by nicotinamide and 5′-methyl nicotinamide. Biochim Biophys Acta 238, 82-85, doi:10.1016/0005-2787(71)90012-8 (1971).
35. Jackson, M. D., Schmidt, M. T., Oppenheimer, N. J. & Denu, J. M. Mechanism of nicotinamide inhibition and transglycosidation by Sir2 histone/protein deacetylases. J Biol Chem 278, 50985-50998, doi:10.1074/jbc.M306552200 (2003).
36. Sauve, A. A. & Schramm, V. L. Sir2 regulation by nicotinamide results from switching between base exchange and deacetylation chemistry. Biochemistry 42, 9249-9256, doi:10.1021/bi0349591 (2003).
37. Bi, T. Q. & Che, X. M. Nampt/PBEF/visfatin and cancer. Cancer Biol Ther 10, 119-125, doi:10.4161/cbt.10.2.12581 (2010).
38. Tian, W. et al. Visfatin, a potential biomarker and prognostic factor for endometrial cancer. Gynecol Oncol 129, 505-512, doi:10.1016/j.ygyno.2013.02.022 (2013).
39. Maldi, E. et al. Nicotinamide phosphoribosyltransferase (NAMPT) is over-expressed in melanoma lesions. Pigment Cell Melanoma Res 26, 144-146, doi:10.1111/pcmr.12037 (2013).
40. Bułdak, R. J. et al. Visfatin affects redox adaptive responses and proliferation in Me45 human malignant melanoma cells: an in vitro study. Oncol Rep 29, 771-778, doi:10.3892/or.2012.2175 (2013).
41. Reddy, P. S. et al. PBEF1/NAmPRTase/Visfatin: a potential malignant astrocytoma/glioblastoma serum marker with prognostic value. Cancer Biol Ther 7, 663-668, doi:10.4161/cbt.7.5.5663 (2008).
42. Ninomiya, S. et al. Possible role of visfatin in hepatoma progression and the effects of branched-chain amino acids on visfatin-induced proliferation in human hepatoma cells. Cancer Prev Res (Phila) 4, 2092-2100, doi:10.1158/1940-6207.Capr-11-0340 (2011).
43. Yagi, M. et al. Association between High Levels of Circulating Chemerin and Colorectal Adenoma in Men. Digestion 101, 571-578, doi:10.1159/000501477 (2020).
44. Nakajima, T. E. et al. Adipocytokine levels in gastric cancer patients: resistin and visfatin as biomarkers of gastric cancer. J Gastroenterol 44, 685-690, doi:10.1007/s00535-009-0063-5 (2009).
45. Yu-Duan, T. et al. Elevated plasma level of visfatin/pre-b cell colony-enhancing factor in male oral squamous cell carcinoma patients. Med Oral Patol Oral Cir Bucal 18, e180-186, doi:10.4317/medoral.18574 (2013).
46. Dalamaga, M. et al. Could serum visfatin be a potential biomarker for postmenopausal breast cancer? Maturitas 71, 301-308, doi:10.1016/j.maturitas.2011.12.013 (2012).
47. Pillai, V. B. et al. Nampt secreted from cardiomyocytes promotes development of cardiac hypertrophy and adverse ventricular remodeling. Am J Physiol Heart Circ Physiol 304, H415-426, doi:10.1152/ajpheart.00468.2012 (2013).
48. Yammani, R. R. & Loeser, R. F. Extracellular nicotinamide phosphoribosyltransferase (NAMPT/visfatin) inhibits insulin-like growth factor-1 signaling and proteoglycan synthesis in human articular chondrocytes. Arthritis Res Ther 14, R23, doi:10.1186/ar3705 (2012).
49. Dahl, T. B. et al. Increased expression of visfatin in macrophages of human unstable carotid and coronary atherosclerosis: possible role in inflammation and plaque destabilization. Circulation 115, 972-980, doi:10.1161/circulationaha.106.665893 (2007).
50. Moschen, A. R. et al. Visfatin, an adipocytokine with proinflammatory and immunomodulating properties. J Immunol 178, 1748-1758, doi:10.4049/jimmunol.178.3.1748 (2007).
51. Kendal, C. E. & Bryant-Greenwood, G. D. Pre-B-cell colony-enhancing factor (PBEF/Visfatin) gene expression is modulated by NF-kappaB and AP-1 in human amniotic epithelial cells. Placenta 28, 305-314, doi:10.1016/j.placenta.2006.03.011 (2007).
52. Kang, Y. S. et al. Visfatin is upregulated in type-2 diabetic rats and targets renal cells. Kidney Int 78, 170-181, doi:10.1038/ki.2010.98 (2010).
53. Sun, H. K. et al. Phosphodiesterase inhibitor improves renal tubulointerstitial hypoxia of the diabetic rat kidney. Korean J Intern Med 27, 163-170, doi:10.3904/kjim.2012.27.2.163 (2012).
54. Jakuš, V., Sándorová, E., Kalninová, J. & Krahulec, B. Monitoring of glycation, oxidative stress and inflammation in relation to the occurrence of vascular complications in patients with type 2 diabetes mellitus. Physiol Res 63, 297-309, doi:10.33549/physiolres.932672 (2014).
55. Kadoglou, N. P. et al. Visfatin (nampt) and ghrelin as novel markers of carotid atherosclerosis in patients with type 2 diabetes. Exp Clin Endocrinol Diabetes 118, 75-80, doi:10.1055/s-0029-1237360 (2010).
56. Kadoglou, N. P. et al. Effects of atorvastatin on apelin, visfatin (nampt), ghrelin and early carotid atherosclerosis in patients with type 2 diabetes. Acta Diabetol 49, 269-276, doi:10.1007/s00592-011-0310-0 (2012).
57. Huang, F., Xiong, X. F., You, S., Wang, Q. X. & Zeng, H. S. [Visfatin upregulates MMP-2 and MMP-9 expressions in human monocytes through activating NF-kappaB]. Zhonghua Xin Xue Guan Bing Za Zhi 38, 455-459 (2010).
58. Montecucco, F. et al. Inhibition of nicotinamide phosphoribosyltransferase reduces neutrophil-mediated injury in myocardial infarction. Antioxid Redox Signal 18, 630-641, doi:10.1089/ars.2011.4487 (2013).
59. Haider, D. G. et al. Free fatty acids normalize a rosiglitazone-induced visfatin release. Am J Physiol Endocrinol Metab 291, E885-890, doi:10.1152/ajpendo.00109.2006 (2006).
60. Moschen, A. R. et al. Visfatin, an adipocytokine with proinflammatory and immunomodulating properties. J Immunol 178, 1748-1758, doi:10.4049/jimmunol.178.3.1748 (2007).
61. Romacho, T. et al. Extracellular PBEF/NAMPT/visfatin activates pro-inflammatory signalling in human vascular smooth muscle cells through nicotinamide phosphoribosyltransferase activity. Diabetologia 52, 2455-2463, doi:10.1007/s00125-009-1509-2 (2009).
62. Rongvaux, A. et al. Nicotinamide phosphoribosyl transferase/pre-B cell colony-enhancing factor/visfatin is required for lymphocyte development and cellular resistance to genotoxic stress. J Immunol 181, 4685-4695, doi:10.4049/jimmunol.181.7.4685 (2008).
63. Nowell, M., Evans, L. & Williams, A. PBEF/NAMPT/visfatin: a promising drug target for treating rheumatoid arthritis? Future Med Chem 4, 751-769, doi:10.4155/fmc.12.34 (2012).
64. Esposito, E. et al. The NAMPT inhibitor FK866 reverts the damage in spinal cord injury. J Neuroinflammation 9, 66, doi:10.1186/1742-2094-9-66 (2012).
65. Nielsen, K. N. et al. NAMPT-mediated NAD(+) biosynthesis is indispensable for adipose tissue plasticity and development of obesity. Mol Metab 11, 178-188, doi:10.1016/j.molmet.2018.02.014 (2018).
66. Nahimana, A. et al. The NAD biosynthesis inhibitor APO866 has potent antitumor activity against hematologic malignancies. Blood 113, 3276-3286, doi:10.1182/blood-2008-08-173369 (2009).
67. Holen, K., Saltz, L. B., Hollywood, E., Burk, K. & Hanauske, A. R. The pharmacokinetics, toxicities, and biologic effects of FK866, a nicotinamide adenine dinucleotide biosynthesis inhibitor. Invest New Drugs 26, 45-51, doi:10.1007/s10637-007-9083-2 (2008).

1. A compound of Formula (I) 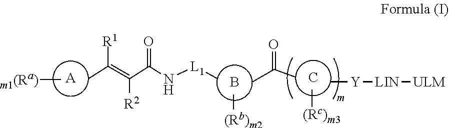
or a salt, enantiomer, stereoisomer, solvate, isotopically enriched analog, prodrug, or polymorph thereof,
wherein ULM is a E3 ligase ligand moiety, LIN is a linker moiety, the remaining moiety of the compound of Formula (I) is a nicotinamide phosphoribosyltransferase (NAMPT) ligand, and ULM is covalently bonded to NAMPT ligand through LIN;
wherein ring A is the following groups: 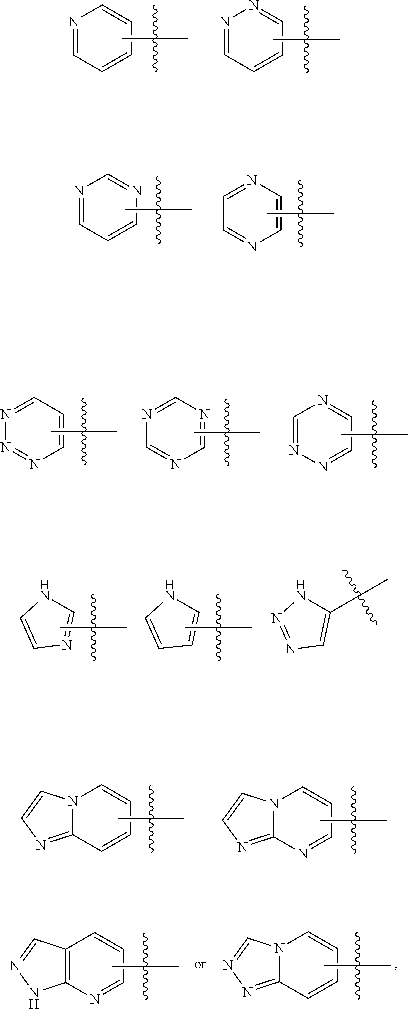
(R^{a})ₘ₁ indicates that the ring A is optionally substituted by m1 R^{a} groups, wherein each R^{a} is independently hydroxyl, amino, halogen, or cyano, and m1 represents an integer of 0, 1, 2, 3, 4, or 5;
R¹ and R² are the same or different and each independently represent H, cyano, or methyl;
L₁ represents substituted or unsubstituted linear C₃₋₆ alkylene group, or substituted or unsubstituted linear C₃₋₆ alkenylene group, wherein the optional substituents of the linear C₃₋₆ alkylene group and the linear C₃₋₆ alkenylene group are selected from the group consisting of C₁₋₃ alkyl, halogen, C₁₋₃ alkoxy, halogenated C₁₋₃ alkyl, C₁₋₃ alkyl-NHC(O)—, C₁₋₃ alkyl-C(O)NH—, cyano, or any combination thereof;
ring B is a 5- to 11-membered heterocyclylene group, and (R^{b})ₘ₂ indicates that ring B is optionally substituted by m2 R^{b} groups, wherein each R^{b} is independently C₁₋₃ alkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, or cyano, and m2 represents an integer of 0, 1, 2, 3, 4, or 5;
ring C is a 5- to 10-membered heteroarylene group or a 6-membered arylene group, and (R^{c})ₘ₃ indicates that ring C is optionally substituted by m3 R^{c} groups, wherein each R^{c} is independently C₁₋₃ alkyl, C₃₋₅ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, or cyano, and m3 represents an integer of 0, 1, 2, 3, 4, or 5;
m represents an integer of 0 or 1; and
Y represents —N(R³)—, where R³ is H or C₁₋₃ alkyl; or
Y represents —O—; or
Y is n connected rings D represented by the following formula: 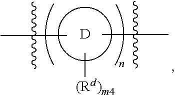
each ring D is independently a 5- to 11-membered heterocyclylene group, and (R^{d})ₘ₄ indicates that each ring D is optionally independently substituted by m4 R^{d} groups, wherein each R^{d} is independently C₁₋₃ alkyl, C₃₋₆ cycloalkyl, hydroxyl, amino, mercapto, halogen, oxo, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, or cyano, and m4 represents an integer of 0, 1, 2, 3, 4, or 5;
n represents an integer of 0, 1, 2, or 3, wherein when n represents an integer of 2 or 3, each ring D can be the same or different, and
where m and n are not both 0 at the same time;
with the proviso that the following compounds are excluded:
the nicotinamide phosphoribosyltransferase (NAMPT) ligand represents the following structure: 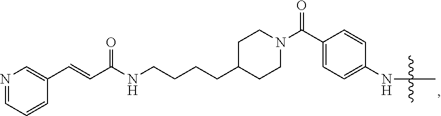 [SMILES: *Nc1ccc(C(=O)N2CCC(CCCCNC(=O)/C=C/c3cccnc3)CC2)cc1]
and
ULM represents a structure of Formula (IV): 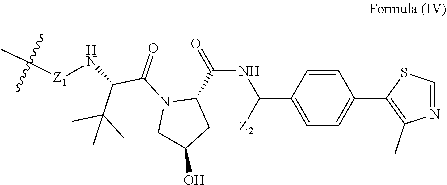
wherein Z₁ represents C(O) or Z₁ represents a bond, and Z₂ represents H or CH₃.

2. The compound of Formula (I) or a salt, enantiomer, stereoisomer, solvate, isotopically enriched analog, prodrug, or polymorph thereof as claimed in claim 1, wherein ring B is a 5- to 11-membered heterocyclylene group containing from 1 to 3 heteroatoms independently selected from the group consisting of nitrogen, oxygen, and sulfur.

3. The compound of Formula (I) or a salt, enantiomer, stereoisomer, solvate, isotopically enriched analog, prodrug, or polymorph thereof as claimed in claim 1, wherein ring B is a 5- to 11-membered heterocyclylene group containing 1 nitrogen atom and optionally containing from 1 to 2 heteroatoms independently selected from the group consisting of nitrogen, oxygen, and sulfur.

4. The compound of Formula (I) or a salt, enantiomer, stereoisomer, solvate, isotopically enriched analog, prodrug, or polymorph thereof as claimed in claim 1, wherein
ring B represents the following groups:
piperidinylene, piperazinylene, morpholinylene, azetidinylene, oxetanylene, pyrrolidinylene, imidazolidylene, pyrazolidylene, tetrahydrofuranylene, tetrahydropyranylene, tetrahydrothienylene, tetrahydrothiopyranylene, oxazolidinylene, thiazolidinylene, thiomorpholinylene, dioxanylene, diazepanylene, or C₇₋₁₁ spiroheterocyclylene; or
ring C represents the following groups:
phenylene, pyridylene, pyrimidinylene, pyrazinylene, pyridazinylene, 1,2,4-triazinylene, 1,3,5-triazinylene, triazolylene, furanylene, oxazolylene, isoxazolylene, oxadiazolylene, thienylene, thiazolylene, isothiazolylene, thiadiazolylene, pyrrolylene, imidazolylene, pyrazolylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2a]pyridylene, 1H-pyrrolo[3,2-b]pyridylene, 1H-pyrrolo[2,3-b]pyridylene, 4H-fluoro[3,2b]pyrrolylene, pyrrolo[2,1-b]thiazolylene, or imidazo[2,1-b]thiazolylene; or
each ring D independently represents the following groups:
piperidinylene, piperazinylene, morpholinylene, azetidinylene, oxetanylene, pyrrolidinylene, imidazolidylene, pyrazolidylene, tetrahydrofuranylene, tetrahydropyranylene, tetrahydrothienylene, tetrahydrothiopyranylene, oxazolidinylene, thiazolidinylene, thiomorpholinylene, dioxanylene, diazepanylene, or C₇₋₁₁ spiroheterocyclylene.

5. The compound of Formula (I) or a salt, enantiomer, stereoisomer, solvate, isotopically enriched analog, prodrug, or polymorph thereof as claimed in claim 1, wherein the compound of Formula (I) is also of Formula (II): 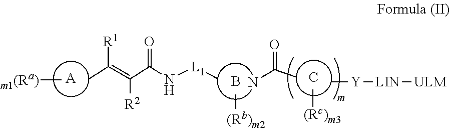
wherein ring B is a 5- to 11-membered heterocyclylene group containing 1 nitrogen atom and optionally containing from 1 to 2 heteroatoms independently selected from the group consisting of nitrogen, oxygen, and sulfur, and (R^{b})ₘ₂ indicates that ring B is optionally substituted by m2 R^{b} groups, wherein each R^{b} is independently C₁₋₃ alkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, or cyano, and m2 represents an integer of 0, 1, 2, 3, 4, or 5; and
ULM, LIN, ring A, (R^{a})ₘ₁, R¹, R², L₁, ring C, (R^{c})ₘ₃, m, and Y are as defined in claim 1.

6. The compound of Formula (I) or a salt, enantiomer, stereoisomer, solvate, isotopically enriched analog, prodrug, or polymorph thereof as claimed in claim 5, wherein
wherein ring B is a 5- to 11-membered heterocyclylene group containing from 1 to 3 nitrogen atoms; or
ring C represents the following groups:
phenylene, pyridylene, pyrimidinylene, pyrazinylene, pyridazinylene, 1,2,4-triazinylene, 1,3,5-triazinylene, triazolylene, furanylene, oxazolylene, isoxazolylene, oxadiazolylene, thienylene, thiazolylene, isothiazolylene, thiadiazolylene, pyrrolylene, imidazolylene, pyrazolylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2a]pyridylene, 1H-pyrrolo[3,2-b]pyridylene, 1H-pyrrolo[2,3-b]pyridylene, 4H-fluoro[3,2b]pyrrolylene, pyrrolo[2,1-b]thiazolylene, or imidazo[2,1-b]thiazolylene; or
each ring D independently represents the following groups:
piperidinylene, piperazinylene, morpholinylene, azetidinylene, oxetanylene, pyrrolidinylene, imidazolidylene, pyrazolidylene, tetrahydrofuranylene, tetrahydropyranylene, tetrahydrothienylene, tetrahydrothiopyranylene, oxazolidinylene, thiazolidinylene, thiomorpholinylene, dioxanylene, diazepanylene, or C₇₋₁₁ spiroheterocyclylene.

7. The compound of Formula (I) or a salt, enantiomer, stereoisomer, solvate, isotopically enriched analog, prodrug, or polymorph thereof as claimed in claim 5, wherein ring B represents piperidinylene, piperazinylene, morpholinylene, azetidinylene, pyrrolidinylene, imidazolidylene, pyrazolidylene, oxazolidinylene, thiazolidinylene, thiomorpholinylene, diazepanylene, or C₇₋₁₁ spiroheterocyclylene containing 1 nitrogen atom and optionally containing from 1 to 2 heteroatoms independently selected from the group consisting of nitrogen, oxygen, and sulfur.

8. The compound of Formula (I) or a salt, enantiomer, stereoisomer, solvate, isotopically enriched analog, prodrug, or polymorph thereof as claimed in claim 4, wherein when ring B represents C₇₋₁₁ spiroheterocyclylene, the C₇₋₁₁ spiroheterocyclylene is the following groups: 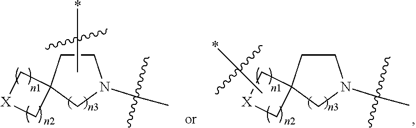
wherein the symbol * indicates the point of attachment to L₁, X represents O, N(R^{e}), S, or CH₂ or X represents a bond, R^{e} represents H or methyl, and n1, n2, and n3 are each independently an integer of 1 or 2.

9. The compound of Formula (I) or a salt, enantiomer, stereoisomer, solvate, isotopically enriched analog, prodrug, or polymorph thereof as claimed in claim 8, wherein when ring B represents C₇₋₁₁ spiroheterocyclylene, the C₇₋₁₁ spiroheterocyclylene is the following groups: 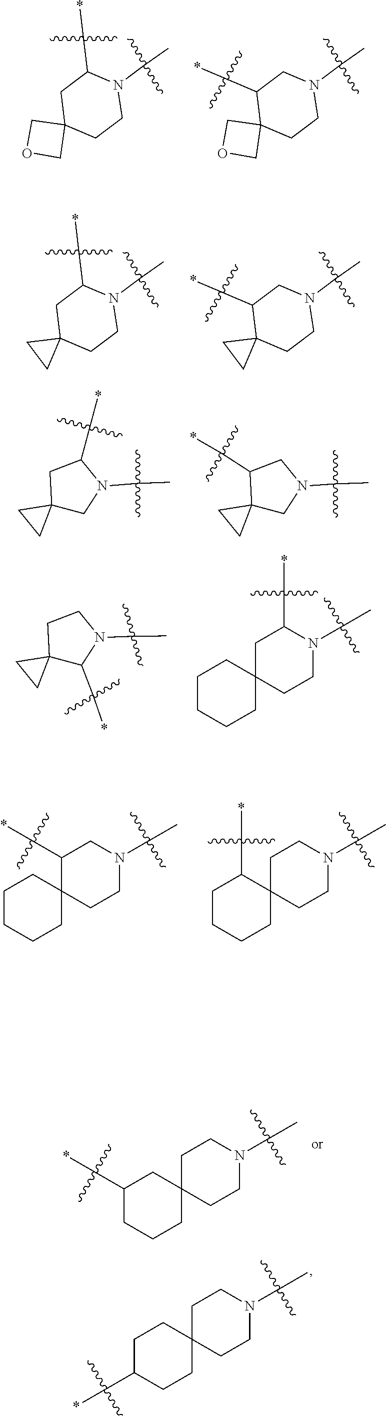
wherein the symbol * indicates the point of attachment to L₁.

10. The compound of Formula (I) or a salt, enantiomer, stereoisomer, solvate, isotopically enriched analog, prodrug, or polymorph thereof as claimed in claim 4, wherein when ring D represents C₇₋₁₁ spiroheterocyclylene, the C₇₋₁₁ spiroheterocyclylene is the following groups: 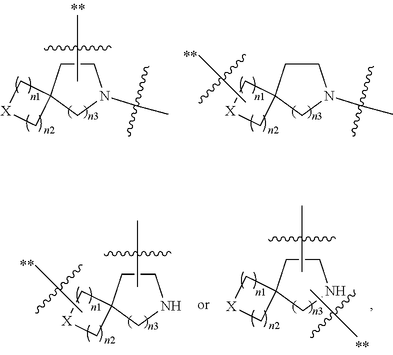
wherein the symbol ** indicates the point of attachment to ring C, X represents O, N(R^{e}), S, or CH₂ or X represents a bond, R^{e} represents H or methyl, and n1, n2, and n3 are each independently an integer of 1 or 2.

11. The compound of Formula (I) or a salt, enantiomer, stereoisomer, solvate, isotopically enriched analog, prodrug, or polymorph thereof as claimed in claim 10, wherein when ring D represents C₇₋₁₁ spiroheterocyclylene, the C₇₋₁₁ spiroheterocyclylene is the following groups: 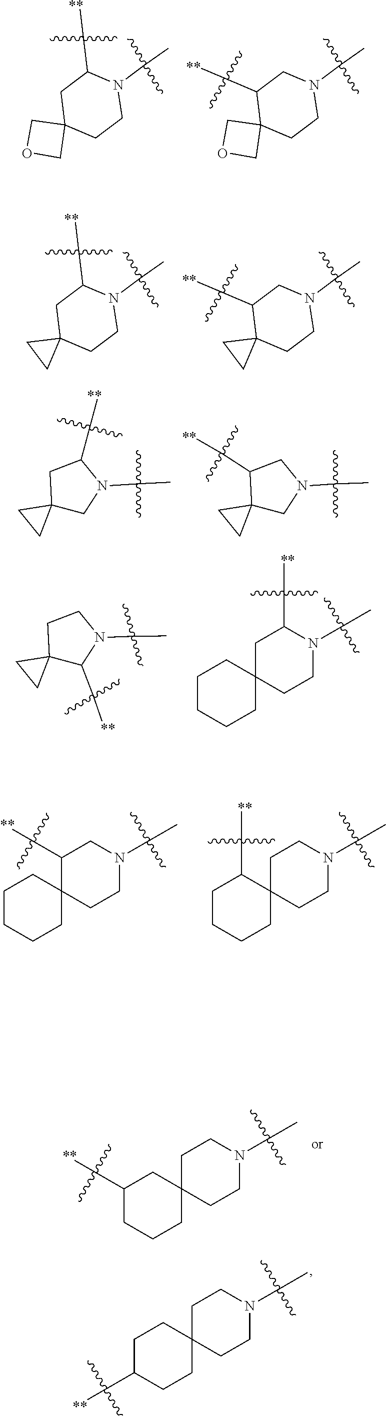
wherein the symbol ** indicates the point of attachment to ring C.

12. The compound of Formula (I) or a salt, enantiomer, stereoisomer, solvate, isotopically enriched analog, prodrug, or polymorph thereof as claimed in claim 1, wherein
ULM represents a structure of Formula (III): 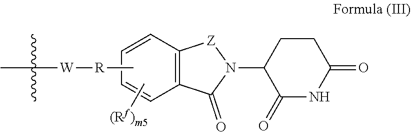
wherein R represents O, N(R⁴), S, alkynylene, alkenylene, or optionally substituted triazolylene, where R⁴ represents H or C₁₋₃ alkyl, or R represents a bond, and W represents optionally substituted phenylene or W represents a bond, wherein when R represents a bond, W represents a bond;
(R^{f})ₘ₅ indicates that the phenyl ring in Formula (III) is optionally substituted by m5 R^{f} groups, wherein each R^{f} is independently halogen, m5 represents an integer of 0, 1, 2, or 3; and Z represents C(O) or CH₂; or
ULM represents a structure of Formula (IV): 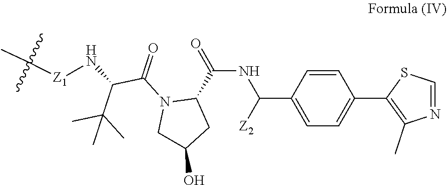
wherein Z₁ represents C(O) or Z₁ represents a bond, and Z₂ represents H or CH₃.

13. The compound of Formula (I) or a salt, enantiomer, stereoisomer, solvate, isotopically enriched analog, prodrug, or polymorph thereof as claimed in claim 12, wherein ULM represents the structure of the following formula: 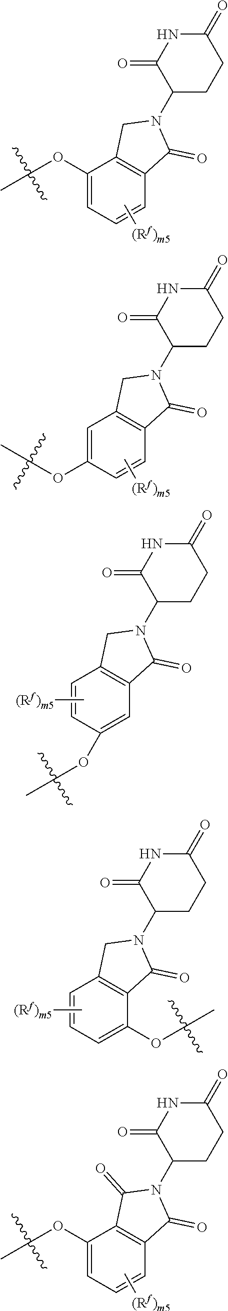 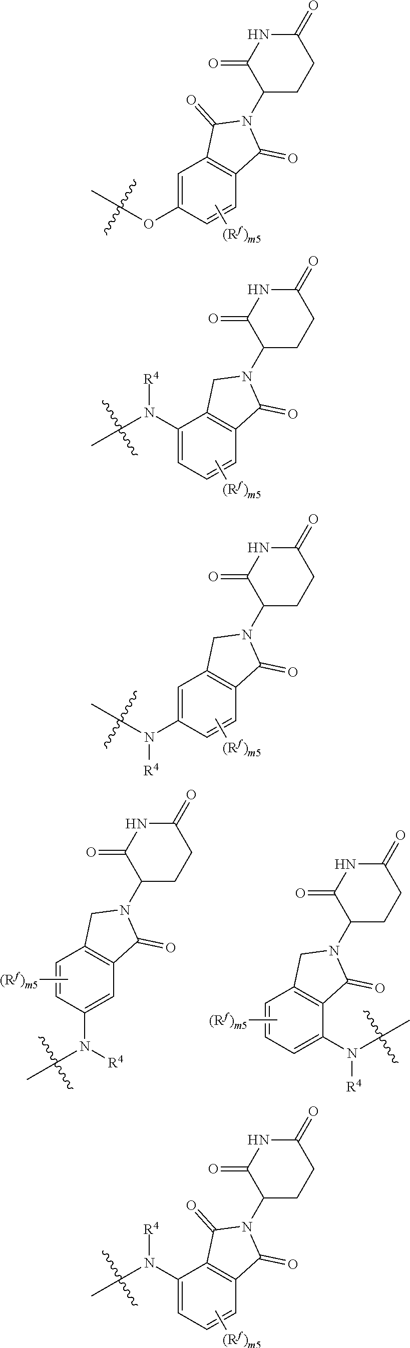 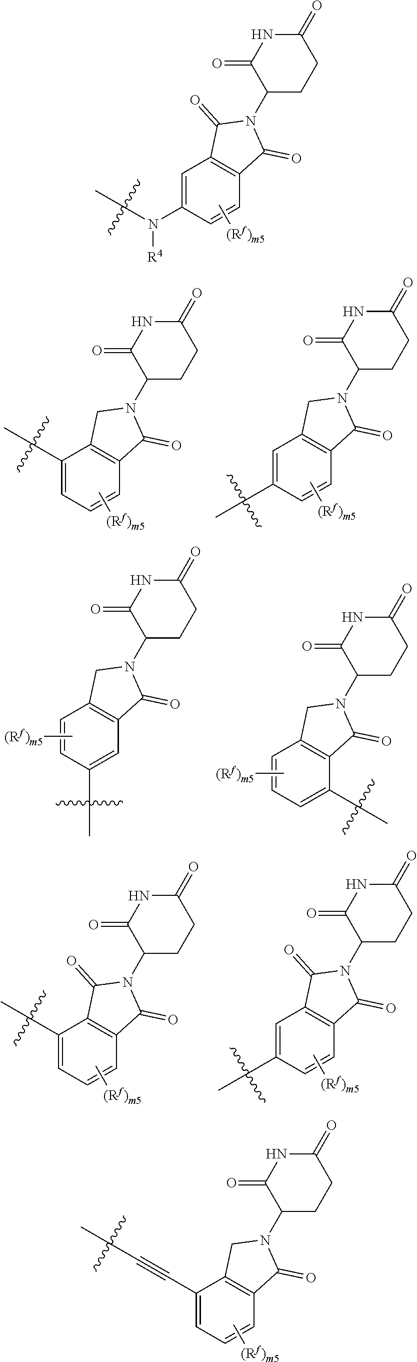 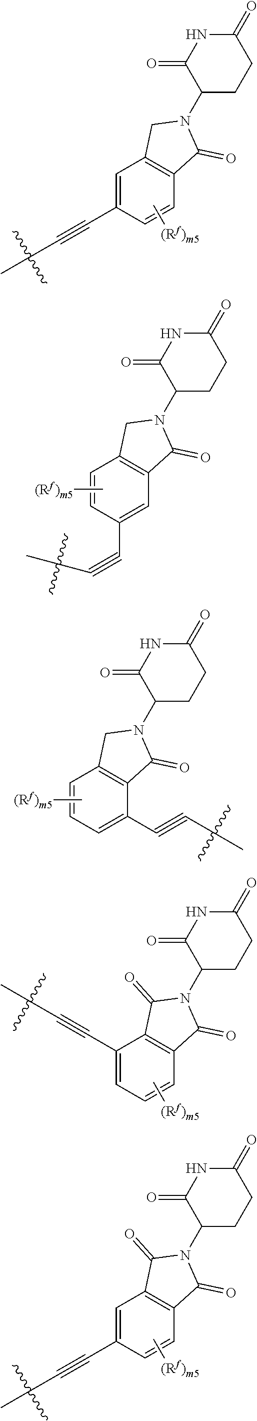 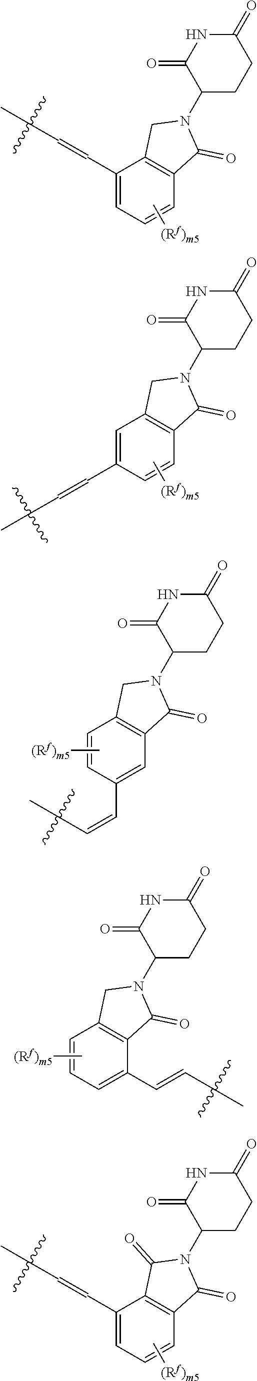 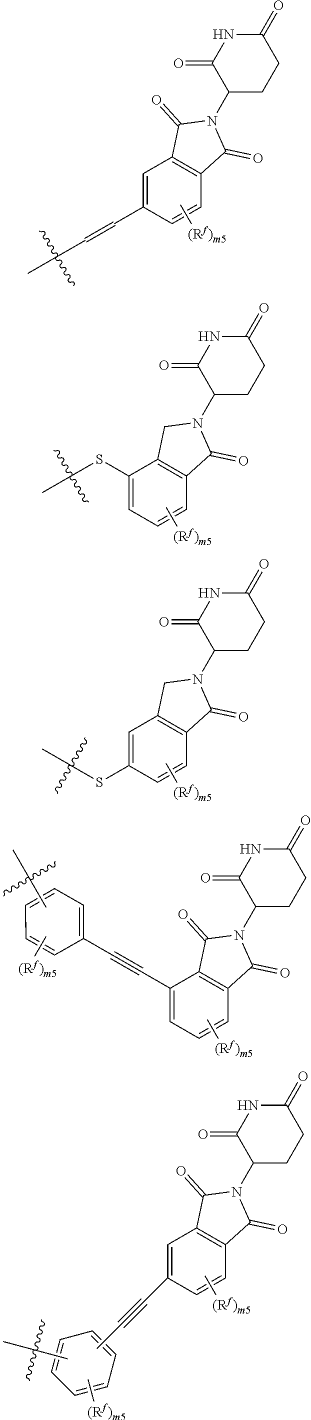 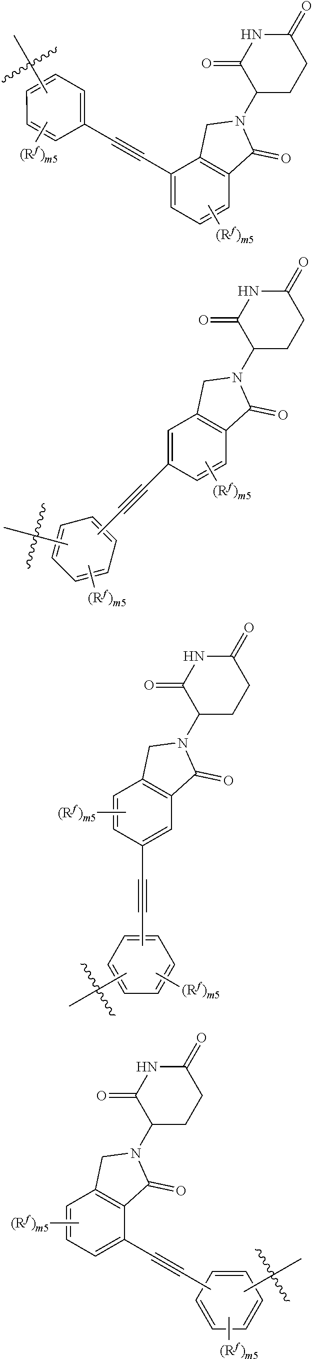 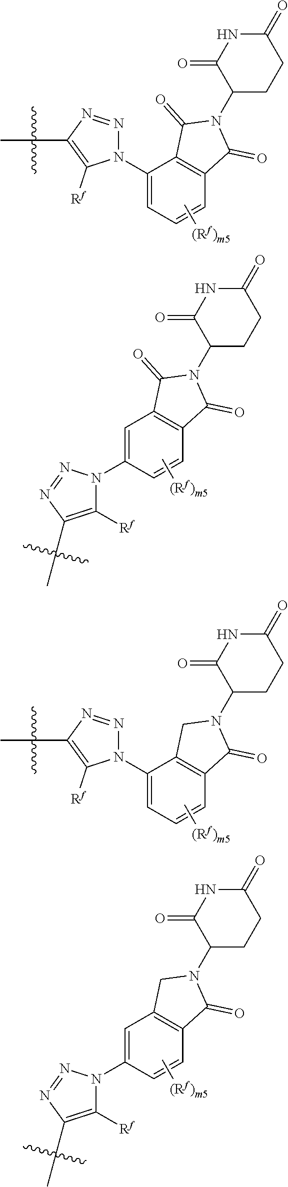 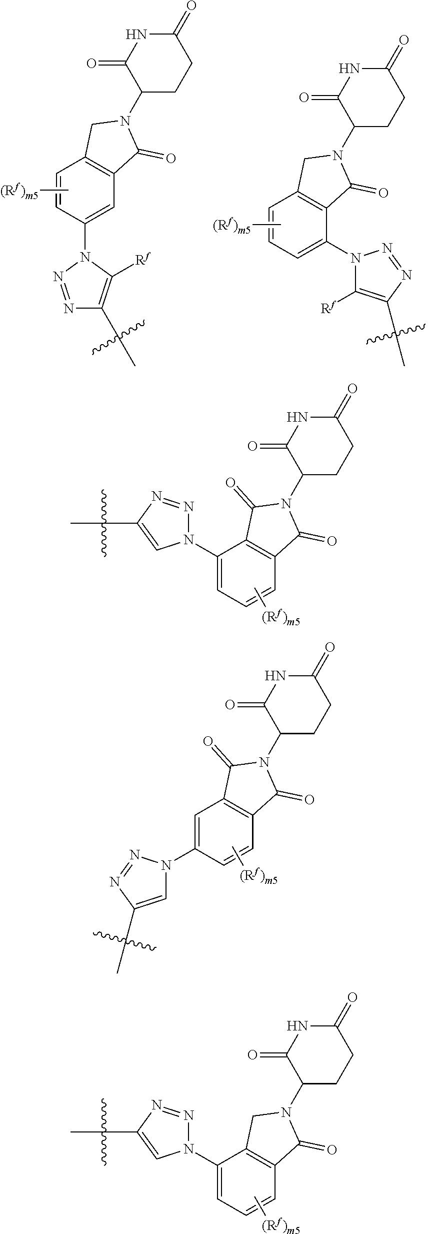 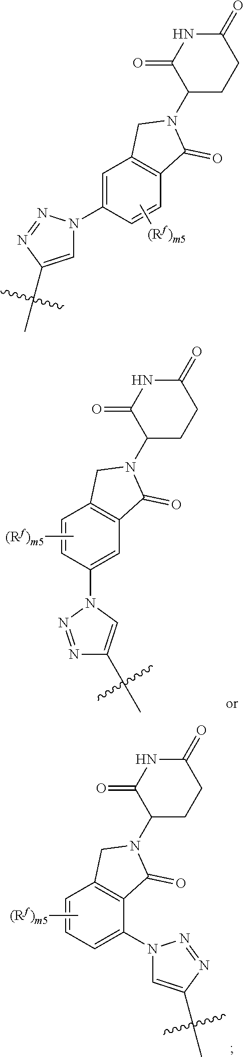
wherein R⁴ represents H or C₁₋₃ alkyl; (R^{f})ₘ₅ indicates that phenyl ring is optionally substituted by m5 R^{f} groups, where each R^{f} is independently halogen, and m5 represents an integer of 0, 1, 2, or 3.

14. The compound of Formula (I) or a salt, enantiomer, stereoisomer, solvate, isotopically enriched analog, prodrug, or polymorph thereof as claimed in claim 12, wherein ULM represents the structure of the following formula: 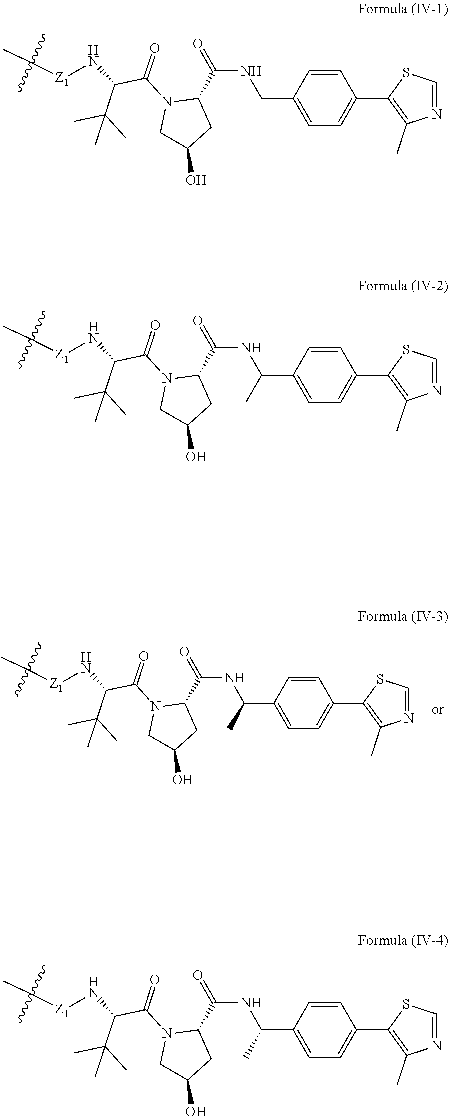
wherein Z₁ represents C(O) or Zn represents a bond.

15. The compound of Formula (I) or a salt, enantiomer, stereoisomer, solvate, isotopically enriched analog, prodrug, or polymorph thereof as claimed in claim 1, wherein LIN is represented by the following formula:
#-U-alkylene,
wherein U represents C(O) or U represents a bond, and the symbol #indicates the point of attachment to Y; and
the alkylene is substituted or unsubstituted methylene, or substituted or unsubstituted linear or branched C₂₋₆₀ alkylene, wherein one or more groups R⁵ and/or one or more groups R⁶ and/or any combination of one or more groups R⁵ and R⁶ are optionally inserted into the backbone carbon chain of the linear or branched C₂₋₆₀ alkylene group;
wherein each R⁵ is independently selected from the group consisting of O, N(R⁷), C(O), C(O)O, OC(O), C(O)N(R⁷), N(R⁷)C(O), or N(R⁷)C(O)N(R⁷), where each R⁷ independently represents H or C₁₋₃ alkyl, and in case that two or more groups R⁵ are inserted into the backbone carbon chain of the linear or branched C₂₋₆₀ alkylene group, the two or more groups R⁵ are not directly connected to each other; and
wherein each R⁶ is independently selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted by a substituent selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

16. The compound of Formula (I) or a salt, enantiomer, stereoisomer, solvate, isotopically enriched analog, prodrug, or polymorph thereof as claimed in claim 15, wherein LIN is represented by the following formula:
#-U-alkylene,
wherein U represents C(O) or U represents a bond, and the symbol #indicates the point of attachment to Y; and
the alkylene is substituted or unsubstituted linear or branched C₁₋₆₀ alkylene, where the substituent is optionally selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

17. The compound of Formula (I) or a salt, enantiomer, stereoisomer, solvate, isotopically enriched analog, prodrug, or polymorph thereof as claimed in claim 16, wherein LIN represents the following groups: #-U—CH₂—, #-U—(CH₂)₂—, #-U—(CH₂)₃—, #-U—(CH₂)₄—, #-U—(CH₂)₅—, #-U—(CH₂)₆—, #-U—(CH₂)₇—, #-U—(CH₂)₈—, #-U—(CH₂)₉—, #-U—(CH₂)₁₀—, #-U—(CH₂)₁₁—, #-U—(CH₂)₁₂—, #-U—(CH₂)₁₃—, #-U—(CH₂)₁₄—, #-U—(CH₂)₁₅—, #-U—(CH₂)₁₆—, #-U—(CH₂)₁₇—, #-U—(CH₂)₁₈—, #-U—(CH₂)₁₉—, #-U—(CH₂)₂₀—, #-U—(CH₂)₂₁—, #-U—(CH₂)₂₂—, #-U—(CH₂)₂₅—, #-U—(CH₂)₃₀—, #-U—(CH₂)₃₅—, #-U—(CH₂)₄₀—, #-U—(CH₂)₄₅—, #-U—(CH₂)₅₀—, #-U—(CH₂)₅₅—, or #-U—(CH₂)₆₀—; wherein a hydrogen atom of one or more CH₂ groups of the groups is optionally replaced with a substituent selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof; and U represents C(O) or U represents a bond, and the symbol #indicates the point of attachment to Y.

18. The compound of Formula (I) or a salt, enantiomer, stereoisomer, solvate, isotopically enriched analog, prodrug, or polymorph thereof as claimed in claim 15, wherein LIN is represented by the following formula:
#-U-alkylene,
wherein U represents C(O) or U represents a bond, and the symbol #indicates the point of attachment to Y; and
the alkylene is substituted or unsubstituted linear or branched C₂₋₆₀ alkylene, wherein one or more groups R⁵ and/or one or more groups R⁶ and/or any combination of one or more groups R⁵ and R⁶ are inserted into the backbone carbon chain of the linear or branched C₂₋₆₀ alkylene group, wherein carbon-carbon bond between one or more pairs of adjacent carbon atoms in the backbone carbon chain is interrupted by the group R⁵, R⁶, or a combination of R⁵ and R⁶;
wherein each R⁵ is independently selected from the group consisting of O, N(R⁷), C(O), C(O)O, OC(O), C(O)N(R⁷), N(R⁷)C(O), or N(R⁷)C(O)N(R⁷), where each R⁷ independently represents H or C₁₋₃ alkyl, and in case that two or more groups R⁵ are inserted into the backbone carbon chain of the linear or branched C₂₋₆₀ alkylene group, the two or more groups R⁵ are not directly connected to each other; and
wherein each R⁶ is independently selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted by a substituent selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

19. The compound of Formula (I) or a salt, enantiomer, stereoisomer, solvate, isotopically enriched analog, prodrug, or polymorph thereof as claimed in claim 1, wherein LIN is represented by the following formula:
#-U—(C(R^{a1})(R^{a2}))ₙ₄—(R⁵(C(R^{a3})(R^{a4}))ₙ₅)ₘ₆—;
#-U—(C(R^{a1})(R^{a2}))ₙ₄—(R⁵(C(R^{a3})(R^{a4}))ₙ₅)ₘ₆—(R⁵(C(R^{a5})(R^{a6}))ₙ₆)ₘ₇—;
#-U—(C(R^{a1})(R^{a2}))ₙ₄—(R⁵(C(R^{a3})(R^{a4}))ₙ₅)ₘ₆—(R⁵(C(R^{a5})(R^{a6}))ₙ₆)ₘ₇—(R⁵(C(R^{a7})(R^{a8}))ₙ₇)ₘ₈—;
#-U—(C(R^{a1})(R^{a2}))ₙ₄—(R⁶(C(R^{a3})(R^{a4}))ₙ₅)ₘ₆—;
#-U—(C(R^{a1})(R^{a2}))ₙ₄—(R⁶(C(R^{a3})(R^{a4}))ₙ₅)ₘ₆—(R⁶(C(R^{a5})(R^{a6}))ₙ₆)ₘ₇—;
#-U—(C(R^{a1})(R^{a2}))ₙ₄—(R⁶(C(R^{a3})(R^{a4}))ₙ₅)ₘ₆—(R⁶(C(R^{a5})(R^{a6}))ₙ₆)ₘ₇—(R⁶(C(R^{a7})(R^{a8}))ₙ₇)ₘ₈—;
#-U—(C(R^{a1})(R^{a2}))ₙ₄—(R⁵—R⁶—(C(R^{a3})(R^{a4}))ₙ₅)ₘ₆—;
#-U—(C(R^{a1})(R^{a2}))ₙ₄—(R⁵(C(R^{a3})(R^{a4}))ₙ₅)ₘ₆—(R⁶(C(R^{a5})(R^{a6}))ₙ₆)ₘ₇—;
#-U—(C(R^{a1})(R^{a2}))ₙ₄—(R⁶—R⁵—(C(R^{a3})(R^{a4}))ₙ₅)ₘ₆—; or
#-U—(C(R^{a1})(R^{a2}))ₙ₄—(R⁶(C(R^{a3})(R^{a4}))ₙ₅)ₘ₆—(R⁵(C(R^{a5})(R^{a6}))ₙ₆)ₘ₇—;
wherein each R⁵ is independently selected from the group consisting of O, N(R⁷), C(O), C(O)O, OC(O), C(O)N(R⁷), N(R⁷)C(O), or N(R⁷)C(O)N(R⁷), where each R⁷ independently represents H or C₁₋₃ alkyl, and in case that two or more groups R⁵ are inserted into the backbone carbon chain of the linear or branched C₂₋₆₀ alkylene group, the two or more groups R⁵ are not directly connected to each other;
wherein each R⁶ is independently selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted by a substituent selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof;
R^{a1}, R^{a2}, R^{a3}, R^{a4}, R^{a5}, R^{a6}, R^{a7} [symbol] R^{a8} each independently represent H, C₁₋₃ alkyl, C₃₋₆ cycloalkyl, hydroxyl, amino, mercapto, halogen, C₁₋₃ alkoxy, C₁₋₃ alkylamino, amino-substituted C₁₋₃ alkylene, halogenated C₁₋₃ alkyl, or cyano,
U represents C(O) or U represents a bond, and the symbol #indicates the point of attachment to Y; and
n4, n5, n6, n7, m6, m7, m8 each independently represent an integer of 1, 2, 3, 4, 5, 6, 7, 8, 9, 10, 11, 12, 13, 14, 15, 16, 17, 18, 19, or 20.

20. The compound of Formula (I) or a salt, enantiomer, stereoisomer, solvate, isotopically enriched analog, prodrug, or polymorph thereof as claimed in claim 1, wherein LIN is represented by the following formula:
#-U—(C(R^{a1})(R^{a2}))ₙ₄—(O(C(R^{a3})(R^{a4}))ₙ₅)ₘ₆—;
#-U—(C(R^{a1})(R^{a2}))ₙ₄—(O(C(R^{a3})(R^{a4}))ₙ₅)ₘ₆—(O(C(R^{a5})(R^{a6}))ₙ₆)ₘ₇—;
#-U—(C(R^{a1})(R^{a2}))ₙ₄—(O(C(R^{a3})(R^{a4}))ₙ₅)ₘ₆—(O(C(R^{a5})(R^{a6}))ₙ₆)ₘ₇—(O(C(R^{a7})(R^{a8}))ₙ₇)ₘ₈—;
#-U—(C(R^{a1})(R^{a2}))ₙ₄—(N(R⁷)(C(R^{a3})(R^{a4}))ₙ₅)ₘ₆—;
#-U—(C(R^{a1})(R^{a2}))ₙ₄—(N(R⁷)(C(R^{a3})(R^{a4}))ₙ₅)ₘ₆—(N(R⁷)(C(R^{a5})(R^{a6}))ₙ₆)ₘ₇—;
#-U—(C(R^{a1})(R^{a2}))ₙ₄—(N(R⁷)(C(R^{a3})(R^{a4}))ₙ₅)ₘ₆—(N(R⁷)(C(R^{a5})(R^{a6}))ₙ₆)ₘ₇—(N(R⁷)(C(R^{a7})(R^{a8}))ₙ₇)ₘ₈—;
#-U—(C(R^{a1})(R^{a2}))ₙ₄—(C(O)N(R⁷)—(C(R^{a3})(R^{a4}))ₙ₅)ₘ₆—;
#-U—(C(R^{a1})(R^{a2}))ₙ₄—(C(O)N(R⁷)—(C(R^{a3})(R^{a4}))ₙ₅)ₘ₆—(C(O)N(R⁷)—(C(R^{a5})(R^{a6}))ₙ₆)ₘ₇—;
#-U—(C(R^{a1})(R^{a2}))ₙ₄—(C(O)N(R⁷)—(C(R^{a3})(R^{a4}))ₙ₅)ₘ₆—(C(O)N(R⁷)—(C(R^{a5})(R^{a6}))ₙ₆)ₘ₇—(C(O)N(R⁷)—(C(R^{a7})(R^{a8}))ₙ₇)ₘ₈—;
#-U—(C(R^{a1})(R^{a2}))ₙ₄—(C(O)N(R⁷)—(C(R^{a3})(R^{a4}))ₙ₅)ₘ₆—(O—(C(R^{a5})(R^{a6}))ₙ₆)ₘ₇—;
#-U—(C(R^{a1})(R^{a2}))ₙ₄—(C(O)N(R⁷)—(C(R^{a3})(R^{a4}))ₙ₅)—(O(C(R^{a5})(R^{a6}))ₙ₆)ₘ₆—;
#-U—(C(R^{a1})(R^{a2}))ₙ₄—C(O)N(R⁷)C(O)—(C(R^{a3})(R^{a4}))ₙ₅)ₘ₆—;
#-U—(C(R^{a1})(R^{a2}))ₙ₄—(N(R⁷)C(O)—(C(R^{a3})(R^{a4}))ₙ₅)ₘ₆—(O—(C(R^{a5})(R^{a6}))ₙ₆)ₘ₇;
#-U—(C(R^{a1})(R^{a2}))ₙ₄—(N(R⁷)C(O)—(C(R^{a3})(R^{a4}))ₙ₅)ₘ₆—(O—(C(R^{a5})(R^{a6}))ₙ₆)ₘ₇—(O—(C(R^{a7})(R^{a8}))ₙ₇)ₘ₈—;
#-U—(C(R^{a1})(R^{a2}))ₙ₄—N(R⁷)C(O)—(C(R^{a3})(R^{a4}))ₙ₅—(O(C(R^{a5})(R^{a6}))ₙ₆)ₘ₆;
#-U—(C(R^{a1})(R^{a2}))ₙ₄—N(R⁷)C(O)N(R⁷)—(C(R^{a3})(R^{a4}))ₙ₅;
#-U—(C(R^{a1})(R^{a2}))ₙ₄—C(O)—(C(R^{a3})(R^{a4}))ₙ₅—;
#-U—(C(R^{a1})(R^{a2}))ₙ₄—CH═CH—(C(R^{a3})(R^{a4}))ₙ₅—;
#-U—(C(R^{a1})(R^{a2}))ₙ₄—C≡C—(C(R^{a3})(R^{a4}))ₙ₅—;
#-U—(C(R^{a1})(R^{a2}))ₙ₄—C≡C—C≡C—(C(R^{a3})(R^{a4}))ₙ₅—;
#-U—(C(R^{a1})(R^{a2}))ₙ₄-(arylene-(C(R^{a3})(R^{a4}))ₙ₅)ₘ₆—;
#-U—(C(R^{a1})(R^{a2}))ₙ₄-(arylene-(C(R^{a3})(R^{a4}))ₙ₅)ₘ₆-arylene-(C(R^{a5})(R^{a6}))ₙ₆—;
#-U—(C(R^{a1})(R^{a2}))ₙ₄-(heterocyclylene-(C(R^{a3})(R^{a4}))ₙ₅)ₘ₆—;
#-U—(C(R^{a1})(R^{a2}))ₙ₄-(heterocyclylene-(C(R^{a3})(R^{a4}))ₙ₅)ₘ₆-(heterocyclylene-(C(R^{a5})(R^{a6}))ₙ₆)ₘ₇—;
#-U—(C(R^{a1})(R^{a2}))ₙ₄-(heteroarylene-(C(R^{a3})(R^{a4}))ₙ₅)ₘ₆—;
#-U—(C(R^{a1})(R^{a2}))ₙ₄-(heteroarylene-(C(R^{a3})(R^{a4}))ₙ₅)ₘ₆-(heteroarylene-(C(R^{a5})(R^{a6}))ₙ₆)ₘ₇—;
#-U—(C(R^{a1})(R^{a2}))ₙ₄-(cycloalkylene-(C(R^{a3})(R^{a4}))ₙ₅)ₘ₆—; or
#-U—(C(R^{a1})(R^{a2}))ₙ₄-(cycloalkylene-(C(R^{a3})(R^{a4}))ₙ₅)ₘ₆-(cycloalkylene-(C(R^{a5})(R^{a6}))ₙ₆)ₘ₇—;
wherein the cycloalkylene, arylene, heterocyclylene, and heteroarylene are each independently optionally substituted by a substituent selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof;
each R⁷ independently represents H or C₁₋₃ alkyl;
R^{a1}, R^{a2}, R^{a3}, R^{a4}, R^{a5}, R^{a6}, R^{a7}, and R^{a8} each independently represent H, C₁₋₃ alkyl, C₃₋₆ cycloalkyl, hydroxyl, amino, mercapto, halogen, C₁₋₃ alkoxy, C₁₋₃ alkylamino, amino-substituted C₁₋₃ alkylene, halogenated C₁₋₃ alkyl, or cyano,
U represents C(O) or U represents a bond, and the symbol #indicates the point of attachment to Y; and
n4, n5, n6, n7, n8, m6, m7, m8 each independently represent an integer of 1, 2, 3, 4, 5, 6, 7, 8, 9, 10, 11, 12, 13, 14, 15, 16, 17, 18, 19, or 20.

21. The compound of Formula (I) or a salt, enantiomer, stereoisomer, solvate, isotopically enriched analog, prodrug, or polymorph thereof as claimed in claim 15, wherein
the cycloalkylene is selected from the group consisting of cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, C₅₋₁₅ spiro-cycloalkylene, adamantanylene, noradamantanylene, bornylene, bicyclo[2.2.1]heptanylene, or bicyclo[2.2.1]heptenylene; and the cycloalkylene is optionally substituted by a substituent selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof;
the arylene is selected from the group consisting of phenylene or naphthylene, and the arylene is optionally substituted by a substituent selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof;
the heterocyclylene is selected from the group consisting of azetidinylene, oxetanylene, pyrrolidinylene, imidazolidylene, pyrazolidylene, tetrahydrofuranylene, tetrahydropyranylene, tetrahydrothienylene, tetrahydrothiopyranylene, oxazolidinylene, thiazolidinylene, piperidinylene, piperazinylene, morpholinylene, thiomorpholinylene, dioxanylene, or diazepanylene; and the heterocyclylene is optionally substituted by a substituent selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof; or
the heteroarylene is selected from the group consisting of furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2a]pyridylene, 1H-pyrrolo[3,2-b]pyridylene, 1H-pyrrolo[2,3-b]pyridylene, 4H-fluoro[3,2b]pyrrolylene, pyrrolo[2,1-b]thiazolylene, or imidazo[2,1-b]thiazolylene; and the heteroarylene is optionally substituted by a substituent selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

22. The compound of Formula (I) or a salt, enantiomer, stereoisomer, solvate, isotopically enriched analog, prodrug, or polymorph thereof as claimed in claim 1, wherein LIN is represented by the following formula:
#-U—CH₂—O—CH₂—, #-U—CH₂—O—(CH₂)₂—, #-U—(CH₂)₁—O—(CH₂)₃—, #-U—(CH₂)₁—O—(CH₂)₄—, #-U—(CH₂)₁—O—(CH₂)₅—, #-U—(CH₂)₁—O—(CH₂)₆—, #-U—(CH₂)₁—O—(CH₂)₇—, #-U—(CH₂)₁—O—(CH₂)₈—, #-U—(CH₂)₁—O—(CH₂)₉—, #-U—(CH₂)₁—O—(CH₂)₁₀—, #-U—(CH₂)₂—O—(CH₂)₁—, #-U—(CH₂)₂—O—(CH₂)₂—, #-U—(CH₂)₂—O—(CH₂)₃—, #-U—(CH₂)₂—O—(CH₂)₄—, #-U—(CH₂)₂—O—(CH₂)₅—, #-U—(CH₂)₂—O—(CH₂)₆—, #-U—(CH₂)₂—O—(CH₂)₇—, #-U—(CH₂)₂—O—(CH₂)₈—, #-U—(CH₂)₂—O—(CH₂)₉—, #-U—(CH₂)₂—O—(CH₂)₁₀—, #-U—(CH₂)₂—O—(CH₂)₁₁—, #-U—(CH₂)₂—O—(CH₂)₁₂—, #-U—(CH₂)₃—O—(CH₂)₁—, #-U—(CH₂)₃—O—(CH₂)₂—, #-U—(CH₂)₃—O—(CH₂)₃—, #-U—(CH₂)₃—O—(CH₂)₄—, #-U—(CH₂)₃—O—(CH₂)₅—, #-U—(CH₂)₃—O—(CH₂)₆—, #-U—(CH₂)₃—O—(CH₂)₇—, #-U—(CH₂)₄—O—(CH₂)₁—, #-U—(CH₂)₄—O—(CH₂)₂—, #-U—(CH₂)₄—O—(CH₂)₃—, #-U—(CH₂)₄—O—(CH₂)₄—, #-U—(CH₂)₄—O—(CH₂)₅—, #-U—(CH₂)₄—O—(CH₂)₆—, #-U—(CH₂)₅—O—(CH₂)₁—, #-U—(CH₂)₅—O—(CH₂)₂—, #-U—(CH₂)₅—O—(CH₂)₃—, #-U—(CH₂)₅—O—(CH₂)₄—, #-U—(CH₂)₅—O—(CH₂)₅—, #-U—(CH₂)₆—O—(CH₂)₁—, #-U—(CH₂)₆—O—(CH₂)₂—, #-U—(CH₂)₆—O—(CH₂)₃—, #-U—(CH₂)₆—O—(CH₂)₄—, #-U—(CH₂)₇—O—(CH₂)₁—, #-U—(CH₂)₇—O—(CH₂)₂—, #-U—(CH₂)₇—O—(CH₂)₃—, #-U—(CH₂)₈—O—(CH₂)₁—, #-U—(CH₂)₈—O—(CH₂)₂—, #-U—CH(CH₃)—O—(CH₂)₁—, #-U—CH(CH₃)—O—(CH₂)₂—, #-U—CH(CH₃)—O—(CH₂)₃—, #-U—CH(CH₃)—O—(CH₂)₄—, #-U—CH(CH₃)—O—(CH₂)₅—, #-U—CH(CH₃)—O—(CH₂)₆—, #-U—CH(CH₃)—O—(CH₂)₇—, #-U—CH(CH₃)—O—(CH₂)₈—, #-U—CH(CH₃)—O—(CH₂)₉—, #-U—CH(CH₃)—O—(CH₂)₁₀—, #-U—CH₂—(O(CH₂)₂)₂—, #-U—CH₂—(O(CH₂)₂)₃—, #-U—CH₂—(O(CH₂)₂)₄—, #-U—CH₂—(O(CH₂)₂)₅—, #-U—CH₂—(O(CH₂)₂)₆—, #-U—CH₂—(O(CH₂)₂)₇—, #- U—CH₂—(O(CH₂)₂)₈—, #-U—CH₂—(O(CH₂)₂)₉—, #-U—CH₂—(O(CH₂)₂)₁₀—, #-U—CH₂—(O(CH₂)₂)₁—OCH₂—, #-U—CH₂—(O(CH₂)₂)₂—OCH₂—, #-U—CH₂—(O(CH₂)₂)₃—OCH₂—, #-U—CH₂—(O(CH₂)₂)₄—OCH₂—, #-U—CH₂—(O(CH₂)₂)₅—OCH₂—, #-U—CH₂—(O(CH₂)₂)₆—OCH₂—, #-U—CH₂—(O(CH₂)₂)₇—OCH₂—, #-U—CH₂—(O(CH₂)₂)₈—OCH₂—, #-U—CH₂—(O(CH₂)₂)₉—OCH₂—, #-U—CH₂—(O(CH₂)₂)₁₀—OCH₂—, #-U—(CH₂)₂—(O(CH₂)₂)₂—, #-U—(CH₂)₂—(O(CH₂)₂)₃—, #-U—(CH₂)₂—(O(CH₂)₂)₄—, #-U—(CH₂)₂—(O(CH₂)₂)₅—, #-U—(CH₂)₂—(O(CH₂)₂)₆—, #-U—(CH₂)₂—(O(CH₂)₂)₇—, #-U—(CH₂)₂—(O(CH₂)₂)₈—, #-U—(CH₂)₂—(O(CH₂)₂)₉—, #-U—(CH₂)₂—(O(CH₂)₂)₁₀—, #-U—(CH₂)₃—(O(CH₂)₂)₂—, #-U—(CH₂)₃—(O(CH₂)₂)₃—, #-U—(CH₂)₃—(O(CH₂)₂)₄—, #-U—(CH₂)₃—(O(CH₂)₂)₅—, #-U—(CH₂)₃—(O(CH₂)₂)₆—, #-U—(CH₂)₃—(O(CH₂)₂)₇—, #-U—(CH₂)₃—(O(CH₂)₂)₈—, #-U—(CH₂)₃—(O(CH₂)₂)₉—, #-U—(CH₂)₃—(O(CH₂)₂)₁₀—, #-U—(CH₂)₄—(O(CH₂)₂)₂—, #-U—(CH₂)₄—(O(CH₂)₂)₃—, #-U—(CH₂)₄—(O(CH₂)₂)₄—, #-U—(CH₂)₄—(O(CH₂)₂)₅—, #-U—(CH₂)₄—(O(CH₂)₂)₆—, #-U—(CH₂)₄—(O(CH₂)₂)₇—, #-U—(CH₂)₄—(O(CH₂)₂)₈—, #-U—(CH₂)₄—(O(CH₂)₂)₉—, #-U—(CH₂)₄—(O(CH₂)₂)₁₀—, #-U—CH₂—(O(CH₂)₃)₂—, #-U—CH₂—(O(CH₂)₃)₃—, #-U—CH₂—(O(CH₂)₃)₄—, #-U—CH₂—(O(CH₂)₃)₅—, #-U—CH₂—(O(CH₂)₃)₆—, #-U—CH₂—(O(CH₂)₃)₇—, #-U—CH₂—(O(CH₂)₃)₈—, #- U—CH₂—(O(CH₂)₃)₉—, #-U—CH₂—(O(CH₂)₃)₁₀—, #-U—(CH₂)₂—(O(CH₂)₃)₂—, #-U—(CH₂)₂—(O(CH₂)₃)₃—, #-U—(CH₂)₂—(O(CH₂)₃)₄—, #-U—(CH₂)₂—(O(CH₂)₃)₅—, #-U—(CH₂)₂—(O(CH₂)₃)₆—, #-U—(CH₂)₂—(O(CH₂)₃)₇—, #-U—(CH₂)₂—(O(CH₂)₃)₈—, #-U—(CH₂)₂—(O(CH₂)₃)₉—, #-U—(CH₂)₂—(O(CH₂)₃)₁₀—, #-U—(CH₂)₃—(O(CH₂)₃)₂—, #-U—(CH₂)₃—(O(CH₂)₃)₃—, #-U—(CH₂)₃—(O(CH₂)₃)₄—, #-U—(CH₂)₃—(O(CH₂)₃)₅—, #-U—(CH₂)₃—(O(CH₂)₃)₆—, #-U—(CH₂)₃—(O(CH₂)₃)₇—, #-U—(CH₂)₃—(O(CH₂)₃)₈—, #-U—(CH₂)₃—(O(CH₂)₃)₉—, #-U—(CH₂)₃—(O(CH₂)₃)₁₀—, #-U—CH₂—O—(CH₂)₂—O—(CH₂)₃—, #-U—CH₂—(O(CH₂)₂)₂—(O(CH₂)₃)₂—, #-U—CH₂—(O(CH₂)₂)₃—(O(CH₂)₃)₃—, #- U—CH₂—(O(CH₂)₂)₄—(O(CH₂)₃)₄—, #-U—CH₂—(O(CH₂)₂)₅—(O(CH₂)₃)₅—, #-U—CH₂—(O(CH₂)₂)₆—(O(CH₂)₃)₆—, #-U—(CH₂)₂—O—(CH₂)₂—O—(CH₂)₃—, #-U—(CH₂)₂—(O(CH₂)₂)₂—(O(CH₂)₃)₂—, #-U—(CH₂)₂—(O(CH₂)₂)₃—(O(CH₂)₃)₃—, #-U—(CH₂)₂—(O(CH₂)₂)₄—(O(CH₂)₃)₄—, #-U—(CH₂)₂—(O(CH₂)₂)₅—(O(CH₂)₃)₅—, #-U—(CH₂)₂—(O(CH₂)₂)₆—(O(CH₂)₃)₆—, #-U—(CH₂)₃—O—(CH₂)₂—O—(CH₂)₃—, #-U—(CH₂)₃—(O(CH₂)₂)₂—(O(CH₂)₃)₂—, #-U—(CH₂)₃—(O(CH₂)₂)₃—(O(CH₂)₃)₃—, #-U—(CH₂)₃—(O(CH₂)₂)₄—(O(CH₂)₃)₄—, #-U—(CH₂)₃—(O(CH₂)₂)₅—(O(CH₂)₃)₅—, #-U—(CH₂)₃—(O(CH₂)₂)₆—(O(CH₂)₃)₆—, #-U—CH₂—O—(CH₂)₃—O—(CH₂)₂—, #-U—CH₂—(O(CH₂)₃)₂—(O(CH₂)₂)₂—, #-U—CH₂—(O(CH₂)₃)₃—(O(CH₂)₂)₃—, #-U—CH₂—(O(CH₂)₃)₄—(O(CH₂)₂)₄—, #-U—CH₂—(O(CH₂)₃)₅—(O(CH₂)₂)₅—, #-U—CH₂—(O(CH₂)₃)₆—(O(CH₂)₂)₆—, #-U—(CH₂)₂—O—(CH₂)₃—O—(CH₂)₂—, #-U—(CH₂)₂—(O(CH₂)₃)₂—(O(CH₂)₂)₂—, #-U—(CH₂)₂—(O(CH₂)₃)₃—(O(CH₂)₂)₃—, #-U—(CH₂)₂—(O(CH₂)₃)₄—(O(CH₂)₂)₄—, #-U—(CH₂)₂—(O(CH₂)₃)₅—(O(CH₂)₂)₅—, #-U—(CH₂)₂—(O(CH₂)₃)₆—(O(CH₂)₂)₆—, #-U—(CH₂)₃—O—(CH₂)₃—O—(CH₂)₂—, #-U—(CH₂)₃—(O(CH₂)₃)₂—(O(CH₂)₂)₂—, #-U—(CH₂)₃—(O(CH₂)₃)₃—(O(CH₂)₂)₃—, #-U—(CH₂)₃—(O(CH₂)₃)₄—(O(CH₂)₂)₄—, #-U—(CH₂)₃—(O(CH₂)₃)₅—(O(CH₂)₂)₅—, #-U—(CH₂)₃—(O(CH₂)₃)₆—(O(CH₂)₂)₆—, #-U—CH₂—O—(CH₂)₂O—CH₂—, #-U—(CH₂)₂—O—(CH₂)₂O—CH₂—, #-U—(CH₂)₂—(O(CH₂)₂)₂—O—(CH₂)₃—, #- U—(CH₂)₂—(O(CH₂)₂)₃—O—(CH₂)₃—, #-U—(CH₂)₂—(O(CH₂)₂)₄—O—(CH₂)₃—, #-U—(CH₂)₅—(O(CH₂)₂)₂—O—(CH₂)₅—, #-U—(CH₂)₅—(O(CH₂)₂)₂—O—(CH₂)₆—, #-U—(CH₂)₁—N(R⁷)—(CH₂)₁—, #-U—(CH₂)₁—N(R⁷)—(CH₂)₂—, #-U—(CH₂)₁—N(R⁷)—(CH₂)₃—, #-U—(CH₂)₁—N(R⁷)—(CH₂)₄—, #- U—(CH₂)₁—N(R⁷)—(CH₂)₈—, #-U—(CH₂)₁—N(R⁷)—(CH₂)₆—, #-U—(CH₂)₁—N(R⁷)—(CH₂)₇—, #- U—(CH₂)₁—N(R⁷)—(CH₂)₈—, #-U—(CH₂)₁—N(R⁷)—(CH₂)₉—, #-U—(CH₂)₁—N(R⁷)—(CH₂)₁₀—, #- U—(CH₂)₂—N(R⁷)—(CH₂)₁—, #-U—(CH₂)₂—N(R⁷)—(CH₂)₂—, #-U—(CH₂)₂—N(R⁷)—(CH₂)₃—, #- U—(CH₂)₂—N(R⁷)—(CH₂)₄—, #-U—(CH₂)₂—N(R⁷)—(CH₂)₅—, #-U—(CH₂)₂—N(R⁷)—(CH₂)₆—, #- U—(CH₂)₂—N(R⁷)—(CH₂)₇—, #-U—(CH₂)₂—N(R⁷)—(CH₂)₈—, #-U—(CH₂)₂—N(R⁷)—(CH₂)₉—, #- U—(CH₂)₂—N(R⁷)—(CH₂)₁₀—, #-U—(CH₂)₂—N(R⁷)—(CH₂)₁₁—, #-U—(CH₂)₂—N(R⁷)—(CH₂)₁₂—, #- U—(CH₂)₃—N(R⁷)—(CH₂)₁—, #-U—(CH₂)₃—N(R⁷)—(CH₂)₂—, #-U—(CH₂)₃—N(R⁷)—(CH₂)₃—, #- U—(CH₂)₄—N(R⁷)—(CH₂)₁—, #-U—(CH₂)₄—N(R⁷)—(CH₂)₂—, #-U—(CH₂)₄—N(R⁷)—(CH₂)₃—, #- U—(CH₂)₄—N(R⁷)—(CH₂)₄—, #-U—(CH₂)₅—N(R⁷)—(CH₂)₁—, #-U—(CH₂)₅—N(R⁷)—(CH₂)₂—, #- U—(CH₂)₅—N(R⁷)—(CH₂)₃—, #-U—(CH₂)₅—N(R⁷)—(CH₂)₄—, #-U—(CH₂)₅—N(R⁷)—(CH₂)₅—, #- U—(CH₂)₆—N(R⁷)—(CH₂)₁—, #-U—(CH₂)₆—N(R⁷)—(CH₂)₂—, #-U—(CH₂)₆—N(R⁷)—(CH₂)₃—, #- U—(CH₂)₇—N(R⁷)—(CH₂)₁—, #-U—(CH₂)₇—N(R⁷)—(CH₂)₂—, #-U—(CH₂)₇—N(R⁷)—(CH₂)₃—, #- U—(CH₂)₈—N(R⁷)—(CH₂)₁—, #-U—(CH₂)₈—N(R⁷)—(CH₂)₂—, #-U—(CH₂)₈—N(R⁷)—(CH₂)₃—, #- U—CH(CH₃)—N(R⁷)—(CH₂)₁—, #-U—CH(CH₃)—N(R⁷)—(CH₂)₂—, #-U—CH(CH₃)—N(R⁷)—(CH₂)₃—, #-U—CH(CH₃)—N(R⁷)—(CH₂)₄—, #-U—CH(CH₃)—N(R⁷)—(CH₂)₅—, #-U—CH(CH₃)—N(R⁷)—(CH₂)₆—, #-U—CH(CH₃)—N(R⁷)—(CH₂)₇—, #-U—CH(CH₃)—N(R⁷)—(CH₂)₈—, #-U—CH(CH₃)—N(R⁷)—(CH₂)₉—, #-U—CH(CH₃)—N(R⁷)—(CH₂)₁₀—, #-U—CH₂C(O)NHCH₂—, #-U—(CH₂)₂C(O)NH(CH₂)₂—, #-U—(CH₂)₂C(O)NH(CH₂)₃—, #-U—(CH₂)₂C(O)NH(CH₂)₄—, #-U—(CH₂)₂C(O)NH(CH₂)₅—, #-U—(CH₂)₃C(O)NH(CH₂)₃—, #-U—(CH₂)₃C(O)NH(CH₂)₄—, #-U—(CH₂)₄C(O)NH(CH₂)₄—, #-U—(CH₂)₅C(O)NH(CH₂)₅—, #-U—(CH₂)₅C(O)NH(CH₂)₇—, #-U—(CH₂)₆C(O)NH(CH₂)₆—, #-U—(CH₂)₇C(O)NH(CH₂)₇—, #-U—(CH₂)₈C(O)NH(CH₂)₈, U—(CH₂)₉C(O)NH(CH₂)₉—, #-U—(CH₂)₁₀C(O)NH(CH₂)₁₀—, #-U—(CH₂)₂C(O)NH(CH₂)₂—O—(CH₂)₂—, #-U—CH₂NHC(O)CH₂—, #-U—(CH₂)₂NHC(O)(CH₂)₂—, #-U—(CH₂)₂NHC(O)(CH₂)₃—, #-U—(CH₂)₂NHC(O)(CH₂)₄—, #-U—(CH₂)₂NHC(O)(CH₂)₅—, #-U—(CH₂)₃NHC(O)(CH₂)₃—, #-U—(CH₂)₃NHC(O)(CH₂)₄—, #-U—(CH₂)₄NHC(O)(CH₂)₄—, #-U—(CH₂)₅NHC(O)(CH₂)₅—, #-U—(CH₂)₃NHC(O)(CH₂)₇—, #-U—(CH₂)₆NHC(O)(CH₂)₆—, #-U—(CH₂)₇NHC(O)(CH₂)₇—, #-U—(CH₂)₈NHC(O)(CH₂)₈, #-U—(CH₂)₉NHC(O)(CH₂)₉—, #-U—(CH₂)₁₀NHC(O)(CH₂)₁₀—, #-U—(CH₂)₄NHC(O)(CH₂)₈—, #-U—(CH₂)₂NHC(O)(CH₂)₂—O—(CH₂)₂—, #-U—(CH₂)₄NHC(O)CH₂—, #-U—CH₂-phenylene- CH₂—, #-U—CH₂-phenylene-(CH₂)₂—, #-U—CH₂-phenylene-(CH₂)₃—, #-U—CH₂-phenylene-(CH₂)₄—, #-U—CH₂-phenylene-(CH₂)₅—, #-U—CH₂-phenylene-(CH₂)₆—, #-U—CH₂-phenylene-(CH₂)₇—, #-U—CH₂-phenylene-(CH₂)₈—, #-U—(CH₂)₂-phenylene-(CH₂)₁—, #-U—(CH₂)₂-phenylene-(CH₂)₂—, #-U—(CH₂)₂-phenylene-(CH₂)₃—, #-U—(CH₂)₂-phenylene-(CH₂)₄—, #-U—(CH₂)₂-phenylene-(CH₂)₅—, #-U—(CH₂)₂-phenylene-(CH₂)₆—, #-U—(CH₂)₂-phenylene-(CH₂)₇—, #-U—(CH₂)₂-phenylene-(CH₂)₈—, #-U—(CH₂)₃-phenylene-CH₂—, #-U—(CH₂)₃-phenylene-(CH₂)₂—, #-U—(CH₂)₃-phenylene-(CH₂)₃—, #-U—(CH₂)₃-phenylene-(CH₂)₄—, #-U—(CH₂)₃-phenylene-(CH₂)₅—, #-U—(CH₂)₃-phenylene-(CH₂)₆—, #-U—(CH₂)₃-phenylene-(CH₂)₇—, #-U—(CH₂)₃-phenylene-(CH₂)₈—, #-U—(CH₂)₄-phenylene-CH₂—, #-U—(CH₂)₄-phenylene-(CH₂)₂—, #-U—(CH₂)₄-phenylene-(CH₂)₃—, #-U—(CH₂)₄-phenylene-(CH₂)₄—, #-U—(CH₂)₄-phenylene-(CH₂)₅—, #-U—(CH₂)₄-phenylene-(CH₂)₆—, #-U—(CH₂)₄-phenylene-(CH₂)₇—, #-U—(CH₂)₄-phenylene-(CH₂)₈—, #-U—(CH₂)₅-phenylene-(CH₂)₁—, #-U—(CH₂)₅-phenylene-(CH₂)₂—, #-U—(CH₂)₅-phenylene-(CH₂)₃—, #-U—(CH₂)₅-phenylene-(CH₂)₄—, #-U—(CH₂)₅-phenylene-(CH₂)₅—, #-U—(CH₂)₅-phenylene-(CH₂)₆—, #-U—(CH₂)₅-phenylene-(CH₂)₇—, #-U—(CH₂)₅-phenylene-(CH₂)₈—, #-U—(CH₂)₆-phenylene-(CH₂)₁—, #-U—(CH₂)₆-phenylene-(CH₂)₂—, #-U—(CH₂)₆-phenylene-(CH₂)₃—, #-U—(CH₂)₆-phenylene-(CH₂)₄—, #-U—(CH₂)₆-phenylene-(CH₂)₅—, #-U—(CH₂)₆-phenylene-(CH₂)₆—, #-U—(CH₂)₆-phenylene-(CH₂)₇—, #-U—(CH₂)₆-phenylene-(CH₂)₈—, #-U—(CH₂)₇-phenylene-(CH₂)₁—, #-U—(CH₂)₇-phenylene-(CH₂)₂—, #-U—(CH₂)₇-phenylene-(CH₂)₃—, #-U—(CH₂)₇-phenylene-(CH₂)₄—, #-U—(CH₂)₇-phenylene-(CH₂)₈—, #-U—(CH₂)₈-phenylene-CH₂—, #-U—(CH₂)₈-phenylene-(CH₂)₂—, #-U—(CH₂)₈-phenylene-(CH₂)₃—, #-U—(CH₂)₈-phenylene-(CH₂)₄—, #-U—(CH₂)₈-phenylene-(CH₂)₅—, #-U—(CH₂)₈-phenylene-(CH₂)₆—, #-U—(CH₂)₈-phenylene-(CH₂)₇—, #-U—(CH₂)₈-phenylene-(CH₂)₈—, #-U—CH₂—N(R⁷)—CH₂-phenylene-CH₂—, #-U—CH₂—N(R⁷)—CH₂-phenylene-(CH₂)₂—, #-U—CH₂—N(R⁷)—CH₂-phenylene-(CH₂)₃—, #-U—CH₂—N(R⁷)—CH₂-phenylene-(CH₂)₄—, #-U—CH₂—N(R⁷)—CH₂-phenylene-(CH₂)₅—, #-U—CH₂—N(R⁷)—CH₂-phenylene-(CH₂)₆—, #-U—CH₂—N(R⁷)—CH₂-phenylene-(CH₂)₇—, #-U—CH₂—N(R⁷)—CH₂-phenylene-(CH₂)₈—, #-U—CH₂—N(R⁷)—(CH₂)₂-phenylene-CH₂—, #-U—CH₂—N(R⁷)—(CH₂)₂-phenylene-(CH₂)₂—, #-U—CH₂—N(R⁷)—(CH₂)₂-phenylene-(CH₂)₃—, #-U—CH₂—N(R⁷)—(CH₂)₂-phenylene-(CH₂)₄—, #-U—CH₂—N(R⁷)—(CH₂)₂-phenylene-(CH₂)₅—, #-U—CH₂—N(R⁷)—(CH₂)₂-phenylene-(CH₂)₆—, #-U—CH₂—N(R⁷)—(CH₂)₂-phenylene-(CH₂)₇—, #-U—CH₂—N(R⁷)—(CH₂)₂-phenylene-(CH₂)₈—, #-U—(CH₂)₂—N(R⁷)—CH₂-phenylene-CH₂—, #-U—(CH₂)₂—N(R⁷)—CH₂-phenylene-(CH₂)₂—, #-U—(CH₂)₂—N(R⁷)—CH₂-phenylene-(CH₂)₃—, #-U—(CH₂)₂—N(R⁷)—CH₂-phenylene-(CH₂)₄—, #-U—(CH₂)₂—N(R⁷)—CH₂-phenylene-(CH₂)₅—, #-U—(CH₂)₂—N(R⁷)—CH₂-phenylene-(CH₂)₆—, #-U—(CH₂)₂—N(R⁷)—CH₂-phenylene-(CH₂)₇—, #-U—(CH₂)₂—N(R⁷)—CH₂-phenylene-(CH₂)₈—, #-U—(CH₂)₃—N(R⁷)—CH₂-phenylene-CH₂—, #-U—(CH₂)₃—N(R⁷)—CH₂-phenylene-(CH₂)₂—, #-U—(CH₂)₃—N(R⁷)—CH₂-phenylene-(CH₂)₃—, #-U—(CH₂)₃—N(R⁷)—CH₂-phenylene-(CH₂)₈—, #-U—(CH₂)₄—N(R⁷)—CH₂-phenylene-CH₂—, #-U—(CH₂)₄—N(R⁷)—CH₂-phenylene-(CH₂)₂—, #-U—(CH₂)₄—N(R⁷)—CH₂-phenylene-(CH₂)₃—, #-U—(CH₂)₄—N(R⁷)—CH₂-phenylene-(CH₂)₈—, #-U—(CH₂)₅—N(R⁷)—CH₂-phenylene-(CH₂)₃—, #-U—(CH₂)₅—N(R⁷)—CH₂-phenylene-(CH₂)₈—, #-U—(CH₂)₆—N(R⁷)—CH₂-phenylene-(CH₂)₃—, #-U—(CH₂)₆—N(R⁷)—CH₂-phenylene-(CH₂)₈—, #-U—(CH₂)₇—N(R⁷)—CH₂-phenylene-(CH₂)₃—, #-U—(CH₂)₇—N(R⁷)—CH₂-phenylene-(CH₂)₈—, #-U—(CH₂)₈—N(R⁷)—CH₂-phenylene-CH₂—, #-U—(CH₂)₈—N(R⁷)—CH₂-phenylene-(CH₂)₂—, #-U—(CH₂)₈—N(R⁷)—CH₂-phenylene-(CH₂)₃—, #-U—(CH₂)₈—N(R⁷)—CH₂-phenylene-(CH₂)₄—, #-U—(CH₂)₈—N(R⁷)—CH₂-phenylene-(CH₂)₅—, #-U—(CH₂)₈—N(R⁷)—CH₂-phenylene-(CH₂)₆—, #-U—(CH₂)₈—N(R⁷)—CH₂-phenylene-(CH₂)₇—, #-U—(CH₂)₈—N(R⁷)—CH₂-phenylene-(CH₂)₈—, #-U—CH₂-piperazinylene-CH₂—, #-U—CH₂-piperazinylene-(CH₂)₂—, #-U—CH₂-piperazinylene-(CH₂)₃—, #-U—CH₂-piperazinylene-(CH₂)₄—, #-U—CH₂-piperazinylene-(CH₂)₅—, #-U—CH₂-piperazinylene-(CH₂)₆—, #-U—CH₂-piperazinylene-(CH₂)₇—, #-U—CH₂-piperazinylene-(CH₂)₈—, #-U—(CH₂)₂-piperazinylene-(CH₂)₁—, #-U—(CH₂)₂-piperazinylene-(CH₂)₂—, #-U—(CH₂)₂-piperazinylene-(CH₂)₃—, #-U—(CH₂)₂-piperazinylene-(CH₂)₄—, #-U—(CH₂)₂-piperazinylene-(CH₂)₅—, #-U—(CH₂)₂-piperazinylene-(CH₂)₆—, #-U—(CH₂)₂-piperazinylene-(CH₂)₇—, #-U—(CH₂)₂-piperazinylene-(CH₂)₈—, #-U—(CH₂)₃-piperazinylene-CH₂—, #-U—(CH₂)₃-piperazinylene-(CH₂)₂—, #-U—(CH₂)₃-piperazinylene-(CH₂)₃—, #-U—(CH₂)₃-piperazinylene-(CH₂)₄—, #-U—(CH₂)₃-piperazinylene-(CH₂)₅—, #-U—(CH₂)₃-piperazinylene-(CH₂)₆—, #-U—(CH₂)₃-piperazinylene-(CH₂)₇—, #-U—(CH₂)₃-piperazinylene-(CH₂)₈—, #-U—(CH₂)₄-piperazinylene-CH₂—, #-U—(CH₂)₄-piperazinylene-(CH₂)₂—, #-U—(CH₂)₄-piperazinylene-(CH₂)₃—, #-U—(CH₂)₄-piperazinylene-(CH₂)₄—, #-U—(CH₂)₄-piperazinylene-(CH₂)₅—, #-U—(CH₂)₄-piperazinylene-(CH₂)₆—, #-U—(CH₂)₄-piperazinylene-(CH₂)₇—, #-U—(CH₂)₄-piperazinylene-(CH₂)₈—, #-U—(CH₂)₅-piperazinylene-(CH₂)₁—, #-U—(CH₂)₅-piperazinylene-(CH₂)₂—, #-U—(CH₂)₅-piperazinylene-(CH₂)₃—, #-U—(CH₂)₅-piperazinylene-(CH₂)₄—, #-U—(CH₂)₅-piperazinylene-(CH₂)₅—, #-U—(CH₂)₅-piperazinylene-(CH₂)₆—, #-U—(CH₂)₅-piperazinylene-(CH₂)₇—, #-U—(CH₂)₅-piperazinylene-(CH₂)₈—, #-U—(CH₂)₆-piperazinylene-(CH₂)₁—, #-U—(CH₂)₆-piperazinylene-(CH₂)₂—, #-U—(CH₂)₆-piperazinylene-(CH₂)₃—, #-U—(CH₂)₆-piperazinylene-(CH₂)₄—, #-U—(CH₂)₆-piperazinylene-(CH₂)₅—, #-U—(CH₂)₆-piperazinylene-(CH₂)₆—, #-U—(CH₂)₆-piperazinylene-(CH₂)₇—, #-U—(CH₂)₆-piperazinylene-(CH₂)₈—, #-U—(CH₂)₇-piperazinylene-(CH₂)₁—, #-U—(CH₂)₇-piperazinylene-(CH₂)₂—, #-U—(CH₂)₇-piperazinylene-(CH₂)₃—, #-U—(CH₂)₇-piperazinylene-(CH₂)₄—, #-U—(CH₂)₇-piperazinylene-(CH₂)₈—, #-U—(CH₂)₈-piperazinylene-CH₂—, #-U—(CH₂)₈-piperazinylene-(CH₂)₂—, #-U—(CH₂)₈-piperazinylene-(CH₂)₃—, #-U—(CH₂)₈-piperazinylene-(CH₂)₄—, #-U—(CH₂)₈-piperazinylene-(CH₂)₅—, #-U—(CH₂)₈-piperazinylene-(CH₂)₆—, #-U—(CH₂)₈-piperazinylene-(CH₂)₇—, or #-U—(CH₂)₈-piperazinylene-(CH₂)₈—;
wherein U represents C(O) or U represents a bond, and the symbol #indicates the point of attachment to Y;
each R⁷ independently represents H or C₁₋₃ alkyl, and
the phenylene and the piperazinylene are each independently optionally substituted by a substituent selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

23. The compound of Formula (I) or a salt, enantiomer, stereoisomer, solvate, isotopically enriched analog, prodrug, or polymorph thereof as claimed in claim 1, wherein
the compound of Formula (I) is also of Formula (V): 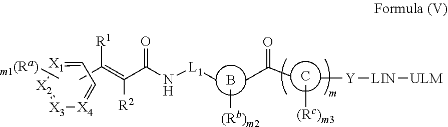
wherein any one, two, or three of X₁, X₂, X₃, and X₄ represent N, and the remaining represent CH; and
ULM, LIN, Y, ring C, (R^{c})ₘ₃, m, ring B, (R^{b})ₘ₂, L₁, R¹, R², (R^{a})ₘ₁ are as defined in any one of claims 1-22; or
the compound of Formula (I) is also of Formula (VI): 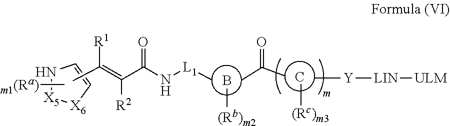
wherein X₅ and X₆ are the same or different and each independently represent CH or N; and
ULM, LIN, Y, ring C, (R^{c})ₘ₃, m, ring B, (R^{b})ₘ₂, L₁, R¹, R², (R^{a})ₘ₁ are as defined in any one of claims 1-22; or
the compound of Formula (I) is also of Formula (VII): 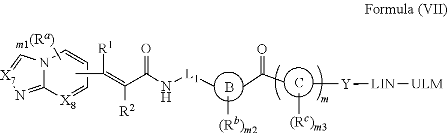
wherein one of X₇ and X₈ represents CH, and the other represents N; or X₇ and X₈ represent CH; and
ULM, LIN, Y, ring C, (R^{c})ₘ₃, m, ring B, (R^{b})ₘ₂, L₁, R¹, R², (R^{a})ₘ₁ are as defined in any one of claims 1-22; or
the compound of Formula (I) is also of Formula (VIII): 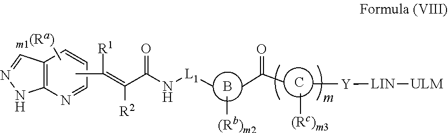
wherein ULM, LIN, Y, ring C, (R^{c})ₘ₃, m, ring B, (R^{b})ₘ₂, L₁, R¹, R², (R^{a})ₘ₁ are as defined in any one of claims 1-22.

24. The compound of Formula (I) or a salt, enantiomer, stereoisomer, solvate, isotopically enriched analog, prodrug, or polymorph thereof as claimed in claim 1, which is selected from:
(E)-N-(4-(1-(4-(4-(3-((2-(2,6-dioxopiperidin-3-yl)-1,3-dioxoisoindolin-4-yl)amino)propanoyl)piperazin-1-yl)benzoyl)piperidin-4-yl)butyl)-3-(pyridin-3-yl)acrylamide;
(E)-N-(4-(1-(4-(4-(4-((2-(2,6-dioxopiperidin-3-yl)-1,3-dioxoisoindolin-4-yl)amino)butanoyl)piperazin-1-yl)benzoyl)piperidin-4-yl)butyl)-3-(pyridin-3-yl)acrylamide;
(E)-N-(4-(1-(4-(4-(5-((2-(2,6-dioxopiperidin-3-yl)-1,3-dioxoisoindolin-4-yl)amino)pentanoyl)piperazin-1-yl)benzoyl)piperidin-4-yl)butyl)-3-(pyridin-3-yl)acrylamide;
(E)-N-(4-(1-(4-(4-((2-(2,6-dioxopiperidin-3-yl)-1-oxoisoindolin-4-yl)glycyl)piperazin-1-yl)benzoyl)piperidin-4-yl)butyl)-3-(pyridin-3-yl)acrylamide;
(E)-N-(4-(1-(4-(4-(4-((2-(2,6-dioxopiperidin-3-yl)-1-oxoisoindolin-4-yl)amino)butanoyl)piperazin-1-yl)benzoyl)piperidin-4-yl)butyl)-3-(pyridin-3-yl)acrylamide;
(E)-N-(4-(1-(4-(4-(5-((2-(2,6-dioxopiperidin-3-yl)-1-oxoisoindolin-4-yl)amino)pentanoyl)piperazin-1-yl)benzoyl)piperidin-4-yl)butyl)-3-(pyridin-3-yl)acrylamide;
(E)-N-(4-(1-(4-(4-(6-((2-(2,6-dioxopiperidin-3-yl)-1-oxoisoindolin-4-yl)amino)hexanoyl)piperazin-1-yl)benzoyl)piperidin-4-yl)butyl)-3-(pyridin-3-yl)acrylamide;
(E)-N-(4-(1-(4-(4-(7-((2-(2,6-dioxopiperidin-3-yl)-1-oxoisoindolin-4-yl)amino)heptanoyl)piperazin-1-yl)benzoyl)piperidin-4-yl)butyl)-3-(pyridin-3-yl)acrylamide;
(E)-N-(4-(1-(4-(4-(7-((2-(2,6-dioxopiperidin-3-yl)-1,3-dioxoisoindolin-4-yl)amino)heptanoyl)piperazin-1-yl)benzoyl)piperidin-4-yl)butyl)-3-(pyridin-3-yl)acrylamide;
(E)-N-(4-(1-(4-(4-(2-((2-(2,6-dioxopiperidin-3-yl)-1-oxoisoindolin-4-yl)thio)acetyl)piperazin-1-yl)benzoyl)piperidin-4-yl)butyl)-3-(pyridin-3-yl)acrylamide;
(E)-N-(4-(1-(4-(4-(5-((2-(2,6-dioxopiperidin-3-yl)-1-oxoisoindolin-4-yl)thio)pentanoyl)piperazin-1-yl)benzoyl)piperidin-4-yl)butyl)-3-(pyridin-3-yl)acrylamide;
(E)-N-(4-(1-(4-(4-(6-((2-(2,6-dioxopiperidin-3-yl)-1-oxoisoindolin-4-yl)thio)hexanoyl)piperazin-1-yl)benzoyl)piperidin-4-yl)butyl)-3-(pyridin-3-yl)acrylamide;
(E)-N-(4-(1-(4-(4-(2-(2-((2-(2,6-dioxopiperidin-3-yl)-1-oxoisoindolin-4-yl)thio)ethoxy)acetyl)piperazin-1-yl)benzoyl)piperidin-4-yl)butyl)-3-(pyridin-3-yl)acrylamide;
(E)-N-(4-(1-(4-(4-(2-(2-(2-((2-(2,6-dioxopiperidin-3-yl)-1-oxoisoindolin-4-yl)thio)ethoxy)ethoxy)acetyl)piperazin-1-yl)benzoyl)piperidin-4-yl)butyl)-3-(pyridin-3-yl)acrylamide;
(E)-N-(4-(1-(4-(4-(2-((2-(2,6-dioxopiperidin-3-yl)-1-oxoisoindolin-4-yl)thio)ethyl)piperazin-1-yl)benzoyl)piperidin-4-yl)butyl)-3-(pyridin-3-yl)acrylamide;
(E)-N-(4-(1-(4-(4-(3-((2-(2,6-dioxopiperidin-3-yl)-1-oxoisoindolin-4-yl)thio)propyl)piperazin-1-yl)benzoyl)piperidin-4-yl)butyl)-3-(pyridin-3-yl)acrylamide;
(E)-N-(4-(1-(4-(4-(4-((2-(2,6-dioxopiperidin-3-yl)-1-oxoisoindolin-4-yl)thio)butyl)piperazin-1-yl)benzoyl)piperidin-4-yl)butyl)-3-(pyridin-3-yl)acrylamide;
(E)-N-(4-(1-(4-(4-(5-((2-(2,6-dioxopiperidin-3-yl)-1-oxoisoindolin-4-yl)thio)pentyl)piperazin-1-yl)benzoyl)piperidin-4-yl)butyl)-3-(pyridin-3-yl)acrylamide;
(E)-N-(4-(1-(4-(4-(6-((2-(2,6-dioxopiperidin-3-yl)-1-oxoisoindolin-4-yl)thio)hexyl)piperazin-1-yl)benzoyl)piperidin-4-yl)butyl)-3-(pyridin-3-yl)acrylamide;
(E)-N-(4-(1-(4-(4-(7-((2-(2,6-dioxopiperidin-3-yl)-1-oxoisoindolin-4-yl)thio)heptyl)piperazin-1-yl)benzoyl)piperidin-4-yl)butyl)-3-(pyridin-3-yl)acrylamide;
(E)-N-(4-(1-(4-(4-(8-((2-(2,6-dioxopiperidin-3-yl)-1-oxoisoindolin-4-yl)thio)octyl)piperazin-1-yl)benzoyl)piperidin-4-yl)butyl)-3-(pyridin-3-yl)acrylamide;
(E)-N-(4-(1-(4-(4-(11-((2-(2,6-dioxopiperidin-3-yl)-1-oxoisoindolin-4-yl)thio)undecyl)piperazin-1-yl)benzoyl)piperidin-4-yl)butyl)-3-(pyridin-3-yl)acrylamide;
(E)-N-(4-(1-(4-(4-(5-(2-(2,6-dioxopiperidin-3-yl)-1-oxoisoindolin-4-yl)pent-4-yn-1-yl)piperazin-1-yl)benzoyl)piperidin-4-yl)butyl)-3-(pyridin-3-yl)acrylamide;
(E)-N-(4-(1-(4-(4-(6-(2-(2,6-dioxopiperidin-3-yl)-1-oxoisoindolin-4-yl)hex-5-yn-1-yl)piperazin-1-yl)benzoyl)piperidin-4-yl)butyl)-3-(pyridin-3-yl)acrylamide;
(E)-N-(4-(1-(4-(4-(7-(2-(2,6-dioxopiperidin-3-yl)-1-oxoisoindolin-4-yl)hept-6-yn-1-yl)piperazin-1-yl)benzoyl)piperidin-4-yl)butyl)-3-(pyridin-3-yl)acrylamide;
(E)-N-(4-(1-(4-(4-(8-(2-(2,6-dioxopiperidin-3-yl)-1-oxoisoindolin-4-yl)oct-7-yn-1-yl)piperazin-1-yl)benzoyl)piperidin-4-yl)butyl)-3-(pyridin-3-yl)acrylamide;
(E)-N-(4-(1-(4-(4-(9-(2-(2,6-dioxopiperidin-3-yl)-1-oxoisoindolin-4-yl)non-8-yn-1-yl)piperazin-1-yl)benzoyl)piperidin-4-yl)butyl)-3-(pyridin-3-yl)acrylamide;
(2S,4R)-1-((S)-3,3-dimethyl-2-(3-oxo-3-(4-(4-(4-(4-((E)-3-(pyridin-3-yl)acrylamido)butyl)piperidine-1-carbonyl)phenyl)piperazin-1-yl)propanamido)butanoyl)-4-hydroxy-N-(4-(4-methylthiazol-5-yl)benzyl)pyrrolidine-2-carboxamide;
(2S,4R)-1-((S)-3,3-dimethyl-2-(4-oxo-4-(4-(4-(4-(4-((E)-3-(pyridin-3-yl)acrylamido)butyl)piperidine-1-carbonyl)phenyl)piperazin-1-yl)butanamido)butanoyl)-4-hydroxy-N-(4-(4-methylthiazol-5-yl)benzyl)pyrrolidine-2-carboxamide;
(2S,4R)-1-((S)-3,3-dimethyl-2-(5-oxo-5-(4-(4-(4-(4-((E)-3-(pyridin-3-yl)acrylamido)butyl)piperidine-1-carbonyl)phenyl)piperazin-1-yl)pentanamido)butanoyl)-4-hydroxy-N-(4-(4-methylthiazol-5-yl)benzyl)pyrrolidine-2-carboxamide;
(2S,4R)-1-((S)-3,3-dimethyl-2-(6-oxo-6-(4-(4-(4-(4-((E)-3-(pyridin-3-yl)acrylamido)butyl)piperidine-1-carbonyl)phenyl)piperazin-1-yl)hexanamido)butanoyl)-4-hydroxy-N-(4-(4-methylthiazol-5-yl)benzyl)pyrrolidine-2-carboxamide;
(2S,4R)-1-((S)-3,3-dimethyl-2-(7-oxo-7-(4-(4-(4-(4-((E)-3-(pyridin-3-yl)acrylamido)butyl)piperidine-1-carbonyl)phenyl)piperazin-1-yl)heptanamido)butanoyl)-4-hydroxy-N-(4-(4-methylthiazol-5-yl)benzyl)pyrrolidine-2-carboxamide;
(2S,4R)-1-((S)-3,3-dimethyl-2-(8-oxo-8-(4-(4-(4-(4-((E)-3-(pyridin-3-yl)acrylamido)butyl)piperidine-1-carbonyl)phenyl)piperazin-1-yl)octanamido)butanoyl)-4-hydroxy-N-(4-(4-methylthiazol-5-yl)benzyl)pyrrolidine-2-carboxamide;
(2S,4R)-1-((S)-3,3-dimethyl-2-(9-oxo-9-(4-(4-(4-(4-((E)-3-(pyridin-3-yl)acrylamido)butyl)piperidine-1-carbonyl)phenyl)piperazin-1-yl)nonanamido)butanoyl)-4-hydroxy-N-(4-(4-methylthiazol-5-yl)benzyl)pyrrolidine-2-carboxamide;
(2S,4R)-1-((S)-3,3-dimethyl-2-(10-oxo-10-(4-(4-(4-(4-((E)-3-(pyridin-3-yl)acrylamido)butyl)piperidine-1-carbonyl)phenyl)piperazin-1-yl)decanamido)butanoyl)-4-hydroxy-N-(4-(4-methylthiazol-5-yl)benzyl)pyrrolidine-2-carboxamide;
(2S,4R)-1-((S)-3,3-dimethyl-2-(11-oxo-11-(4-(4-(4-(4-((E)-3-(pyridin-3-yl)acrylamido)butyl)piperidine-1-carbonyl)phenyl)piperazin-1-yl)undecanamido)butanoyl)-4-hydroxy-N-(4-(4-methylthiazol-5-yl)benzyl)pyrrolidine-2-carboxamide;
(2S,4R)-1-((S)-3,3-dimethyl-2-(2-(2-(2-oxo-2-(4-(4-(4-(4-((E)-3-(pyridin-3-yl)acrylamido)butyl)piperidine-1-carbonyl)phenyl)piperazin-1-yl)ethoxy)ethoxy)acetamido)butanoyl)-4-hydroxy-N-(4-(4-methylthiazol-5-yl)benzyl)pyrrolidine-2-carboxamide;
(2S,4R)-1-((S)-3,3-dimethyl-2-(3-(2-(3-oxo-3-(4-(4-(4-(4-((E)-3-(pyridin-3-yl)acrylamido)butyl)piperidine-1-carbonyl)phenyl)piperazin-1-yl)propoxy)ethoxy)propanamido)butanoyl)-4-hydroxy-N-(4-(4-methylthiazol-5-yl)benzyl)pyrrolidine-2-carboxamide;
(2S,4R)-1-((S)-2-(tert-butyl)-4,16-dioxo-16-(4-(4-(4-(4-((E)-3-(pyridin-3-yl)acrylamido)butyl)piperidine-1-carbonyl)phenyl)piperazin-1-yl)-7,10,13-trioxa-3-azahexadecanoyl)-4-hydroxy-N-(4-(4-methylthiazol-5-yl)benzyl)pyrrolidine-2-carboxamide;
(2S,4R)-1-((S)-2-(tert-butyl)-4,19-dioxo-19-(4-(4-(4-(4-((E)-3-(pyridin-3-yl)acrylamido)butyl)piperidine-1-carbonyl)phenyl)piperazin-1-yl)-7,10,13,16-tetraoxa-3-azanonadecanoyl)-4-hydroxy-N-(4-(4-methylthiazol-5-yl)benzyl)pyrrolidine-2-carboxamide;
(2S,4R)-1-((S)-2-(tert-butyl)-4,22-dioxo-22-(4-(4-(4-(4-((E)-3-(pyridin-3-yl)acrylamido)butyl)piperidine-1-carbonyl)phenyl)piperazin-1-yl)-7,10,13,16,19-pentaoxa-3-azadocosanoyl)-4-hydroxy-N-(4-(4-methylthiazol-5-yl)benzyl)pyrrolidine-2-carboxamide;
(2S,4R)-1-((S)-3,3-dimethyl-2-(16-oxo-16-(4-(4-(4-(4-((E)-3-(pyridin-3-yl)acrylamido)butyl)piperidine-1-carbonyl)phenyl)piperazin-1-yl)hexadecanamido)butanoyl)-4-hydroxy-N-(4-(4-methylthiazol-5-yl)benzyl)pyrrolidine-2-carboxamide;
(E)-N-(4-(1-(4-(4-(4-((2-(2,6-dioxopiperidin-3-yl)-1-oxoisoindolin-4-yl)oxy)butyl)piperazin-1-yl)benzoyl)piperidin-4-yl)butyl)-3-(pyridin-3-yl)acrylamide;
(E)-N-(4-(1-(4-(4-(8-((2-(2,6-dioxopiperidin-3-yl)-1-oxoisoindolin-4-yl)oxy)octyl)piperazin-1-yl)benzoyl)piperidin-4-yl)butyl)-3-(pyridin-3-yl)acrylamide;
(E)-N-(4-(4-(4-(4-(7-(2-(2,6-dioxopiperidin-3-yl)-1-oxoisoindolin-4-yl)hept-6-yn-1-yl)piperazin-1-yl)benzoyl)piperazin-1-yl)butyl)-3-(pyridin-3-yl)acrylamide;
(E)-N-(4-(1-(4-(4-(2-((2-(2,6-dioxopiperidin-3-yl)-1,3-dioxoisoindolin-4-yl)thio)ethyl)piperazin-1-yl)benzoyl)piperidin-4-yl)butyl)-3-(pyridin-3-yl)acrylamide;
(E)-N-(4-(1-(4-(4-(3-((2-(2,6-dioxopiperidin-3-yl)-1,3-dioxoisoindolin-4-yl)thio)propyl)piperazin-1-yl)benzoyl)piperidin-4-yl)butyl)-3-(pyridin-3-yl)acrylamide;
(E)-N-(4-(1-(4-(4-(4-((2-(2,6-dioxopiperidin-3-yl)-1,3-dioxoisoindolin-4-yl)thio)butyl)piperazin-1-yl)benzoyl)piperidin-4-yl)butyl)-3-(pyridin-3-yl)acrylamide;
(E)-N-(4-(1-(4-(4-(5-((2-(2,6-dioxopiperidin-3-yl)-1,3-dioxoisoindolin-4-yl)thio)pentyl)piperazin-1-yl)benzoyl)piperidin-4-yl)butyl)-3-(pyridin-3-yl)acrylamide;
(E)-N-(4-(1-(4-(4-(6-((2-(2,6-dioxopiperidin-3-yl)-1,3-dioxoisoindolin-4-yl)thio)hexyl)piperazin-1-yl)benzoyl)piperidin-4-yl)butyl)-3-(pyridin-3-yl)acrylamide;
(E)-N-(4-(1-(4-(4-(7-((2-(2,6-dioxopiperidin-3-yl)-1,3-dioxoisoindolin-4-yl)thio)heptyl)piperazin-1-yl)benzoyl)piperidin-4-yl)butyl)-3-(pyridin-3-yl)acrylamide;
(E)-N-(4-(1-(4-(4-(8-((2-(2,6-dioxopiperidin-3-yl)-1,3-dioxoisoindolin-4-yl)thio)octyl)piperazin-1-yl)benzoyl)piperidin-4-yl)butyl)-3-(pyridin-3-yl)acrylamide;
(E)-N-(4-(1-(4-(4-(2-((2-(2,6-dioxopiperidin-3-yl)-1,3-dioxoisoindolin-4-yl)thio)acetyl)piperazin-1-yl)benzoyl)piperidin-4-yl)butyl)-3-(pyridin-3-yl)acrylamide;
(E)-N-(4-(1-(4-(4-(3-((2-(2,6-dioxopiperidin-3-yl)-1,3-dioxoisoindolin-4-yl)thio)propanoyl)piperazin-1-yl)benzoyl)piperidin-4-yl)butyl)-3-(pyridin-3-yl)acrylamide;
(E)-N-(4-(1-(4-(4-(5-((2-(2,6-dioxopiperidin-3-yl)-1,3-dioxoisoindolin-4-yl)thio)pentanoyl)piperazin-1-yl)benzoyl)piperidin-4-yl)butyl)-3-(pyridin-3-yl)acrylamide;
(E)-N-(4-(1-(4-(4-(6-((2-(2,6-dioxopiperidin-3-yl)-1,3-dioxoisoindolin-4-yl)thio)hexanoyl)piperazin-1-yl)benzoyl)piperidin-4-yl)butyl)-3-(pyridin-3-yl)acrylamide;
(E)-N-(4-(1-(4-(4-(7-((2-(2,6-dioxopiperidin-3-yl)-1,3-dioxoisoindolin-4-yl)thio)heptanoyl)piperazin-1-yl)benzoyl)piperidin-4-yl)butyl)-3-(pyridin-3-yl)acrylamide;
(E)-N-(4-(1-(4-(4-(8-((2-(2,6-dioxopiperidin-3-yl)-1,3-dioxoisoindolin-4-yl)thio)octanoyl)piperazin-1-yl)benzoyl)piperidin-4-yl)butyl)-3-(pyridin-3-yl)acrylamide;
(E)-N-(4-(1-(4-(4-(2-((2-(2,6-dioxopiperidin-3-yl)-1,3-dioxoisoindolin-4-yl)oxy)acetyl)piperazin-1-yl)benzoyl)piperidin-4-yl)butyl)-3-(pyridin-3-yl)acrylamide;
(E)-N-(4-(1-(4-(4-(5-((2-(2,6-dioxopiperidin-3-yl)-1,3-dioxoisoindolin-4-yl)oxy)pentanoyl)piperazin-1-yl)benzoyl)piperidin-4-yl)butyl)-3-(pyridin-3-yl)acrylamide;
(E)-N-(4-(1-(4-(4-(6-((2-(2,6-dioxopiperidin-3-yl)-1,3-dioxoisoindolin-4-yl)oxy)hexanoyl)piperazin-1-yl)benzoyl)piperidin-4-yl)butyl)-3-(pyridin-3-yl)acrylamide;
(E)-N-(4-(1-(4-(4-(7-((2-(2,6-dioxopiperidin-3-yl)-1,3-dioxoisoindolin-4-yl)oxy)heptanoyl)piperazin-1-yl)benzoyl)piperidin-4-yl)butyl)-3-(pyridin-3-yl)acrylamide;
(E)-N-(4-(1-(4-(1-(7-(2-(2,6-dioxopiperidin-3-yl)-1-oxoisoindolin-4-yl)hept-6-yn-1-yl)piperidin-4-yl)benzoyl)piperidin-4-yl)butyl)-3-(pyridin-3-yl)acrylamide;
(E)-N-(4-(1-(4-(1-(8-(2-(2,6-dioxopiperidin-3-yl)-1-oxoisoindolin-4-yl)oct-7-yn-1-yl)piperidin-4-yl)benzoyl)piperidin-4-yl)butyl)-3-(pyridin-3-yl)acrylamide;
(E)-N-(4-(1-(4-(1-(4-((2-(2,6-dioxopiperidin-3-yl)-1-oxoisoindolin-4-yl)thio)butyl)piperidin-4-yl)benzoyl)piperidin-4-yl)butyl)-3-(pyridin-3-yl)acrylamide;
(E)-N-(4-(1-(6-(4-(4-(7-(2-(2,6-dioxopiperidin-3-yl)-1-oxoisoindolin-4-yl)hept-6-yn-1-yl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(8-(2-(2,6-dioxopiperidin-3-yl)-1-oxoisoindolin-4-yl)oct-7-yn-1-yl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(4-((2-(2,6-dioxopiperidin-3-yl)-1-oxoisoindolin-4-yl)thio)butyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(7-(2-(2,6-dioxopiperidin-3-yl)-1-oxoisoindolin-4-yl)hept-6-yn-1-yl)piperazin-1-yl)pyridazine-3-carbonyl)piperidin-4-yl)butyl)-3-(pyridin-3-yl)acrylamide;
(E)-N-(4-(1-(6-(4-(8-(2-(2,6-dioxopiperidin-3-yl)-1-oxoisoindolin-4-yl)oct-7-yn-1-yl)piperazin-1-yl)pyridazine-3-carbonyl)piperidin-4-yl)butyl)-3-(pyridin-3-yl)acrylamide;
(E)-N-(4-(1-(6-(4-(4-((2-(2,6-dioxopiperidin-3-yl)-1-oxoisoindolin-4-yl)thio)butyl)piperazin-1-yl)pyridazine-3-carbonyl)piperidin-4-yl)butyl)-3-(pyridin-3-yl)acrylamide;
(E)-N-(4-(1-(6-(4-(7-(2-(2,6-dioxopiperidin-3-yl)-1-oxoisoindolin-4-yl)hept-6-yn-1-yl)piperazin-1-yl)nicotinoyl)piperidin-4-yl)butyl)-3-(pyridin-3-yl)acrylamide;
(E)-N-(4-(1-(6-(4-(8-(2-(2,6-dioxopiperidin-3-yl)-1-oxoisoindolin-4-yl)oct-7-yn-1-yl)piperazin-1-yl)nicotinoyl)piperidin-4-yl)butyl)-3-(pyridin-3-yl)acrylamide;
(E)-N-(4-(1-(6-(4-(4-((2-(2,6-dioxopiperidin-3-yl)-1-oxoisoindolin-4-yl)thio)butyl)piperazin-1-yl)nicotinoyl)piperidin-4-yl)butyl)-3-(pyridin-3-yl)acrylamide;
(E)-N-(4-(1-(4-(4-(3-(2-((2-(2,6-dioxopiperidin-3-yl)-1,3-dioxoisoindolin-4-yl)amino)ethoxy)propanoyl)piperazin-1-yl)benzoyl)piperidin-4-yl)butyl)-3-(pyridin-3-yl)acrylamide;
(E)-N-(4-(1-(4-(4-(3-(2-(2-((2-(2,6-dioxopiperidin-3-yl)-1,3-dioxoisoindolin-4-yl)amino)ethoxy)ethoxy)propanoyl)piperazin-1-yl)benzoyl)piperidin-4-yl)butyl)-3-(pyridin-3-yl)acrylamide;
(E)-N-(4-(1-(4-(4-(5-(2-(2,6-dioxopiperidin-3-yl)-1,3-dioxoisoindolin-4-yl)pent-4-yn-1-yl)piperazin-1-yl)benzoyl)piperidin-4-yl)butyl)-3-(pyridin-3-yl)acrylamide;
(E)-N-(4-(1-(4-(4-(6-(2-(2,6-dioxopiperidin-3-yl)-1,3-dioxoisoindolin-4-yl)hex-5-yn-1-yl)piperazin-1-yl)benzoyl)piperidin-4-yl)butyl)-3-(pyridin-3-yl)acrylamide;
(E)-N-(4-(1-(4-(4-(7-(2-(2,6-dioxopiperidin-3-yl)-1,3-dioxoisoindolin-4-yl)hept-6-yn-1-yl)piperazin-1-yl)benzoyl)piperidin-4-yl)butyl)-3-(pyridin-3-yl)acrylamide;
(E)-N-(4-(1-(4-(4-(8-(2-(2,6-dioxopiperidin-3-yl)-1,3-dioxoisoindolin-4-yl)oct-7-yn-1-yl)piperazin-1-yl)benzoyl)piperidin-4-yl)butyl)-3-(pyridin-3-yl)acrylamide;
(E)-N-(4-(1-(4-(4-(3-(2-((2-(2,6-dioxopiperidin-3-yl)-1,3-dioxoisoindolin-4-yl)thio)ethoxy)propanoyl)piperazin-1-yl)benzoyl)piperidin-4-yl)butyl)-3-(pyridin-3-yl)acrylamide;
(E)-N-(4-(1-(4-(4-(2-(2-((2-(2,6-dioxopiperidin-3-yl)-1,3-dioxoisoindolin-4-yl)thio)ethoxy)acetyl)piperazin-1-yl)benzoyl)piperidin-4-yl)butyl)-3-(pyridin-3-yl)acrylamide;
(E)-N-(4-(1-(4-(4-(3-(2-(2-((2-(2,6-dioxopiperidin-3-yl)-1,3-dioxoisoindolin-4-yl)thio)ethoxy)ethoxy)propanoyl)piperazin-1-yl)benzoyl)piperidin-4-yl)butyl)-3-(pyridin-3-yl)acrylamide;
(E)-N-(4-(1-(4-(4-(2-(2-(2-((2-(2,6-dioxopiperidin-3-yl)-1,3-dioxoisoindolin-4-yl)thio)ethoxy)ethoxy)acetyl)piperazin-1-yl)benzoyl)piperidin-4-yl)butyl)-3-(pyridin-3-yl)acrylamide;
(E)-N-(4-(1-(4-(4-(3-(2-(2-(2-((2-(2,6-dioxopiperidin-3-yl)-1,3-dioxoisoindolin-4-yl)thio)ethoxy)ethoxy)ethoxy)propanoyl)piperazin-1-yl)benzoyl)piperidin-4-yl)butyl)-3-(pyridin-3-yl)acrylamide;
(E)-N-(4-(1-(4-(4-(2-(2-(2-(2-((2-(2,6-dioxopiperidin-3-yl)-1,3-dioxoisoindolin-4-yl)thio)ethoxy)ethoxy)ethoxy)acetyl)piperazin-1-yl)benzoyl)piperidin-4-yl)butyl)-3-(pyridin-3-yl)acrylamide;
(E)-N-(4-(1-(4-(4-(14-((2-(2,6-dioxopiperidin-3-yl)-1,3-dioxoisoindolin-4-yl)thio)-3,6,9,12-tetraoxatetradecanoyl)piperazin-1-yl)benzoyl)piperidin-4-yl)butyl)-3-(pyridin-3-yl)acrylamide;
(E)-N-(4-(1-(4-(4-(17-((2-(2,6-dioxopiperidin-3-yl)-1,3-dioxoisoindolin-4-yl)thio)-3,6,9,12,15-pentaoxaheptadecanoyl)piperazin-1-yl)benzoyl)piperidin-4-yl)butyl)-3-(pyridin-3-yl)acrylamide;
(E)-N-(4-(1-(4-(4-(17-((2-(2,6-dioxopiperidin-3-yl)-1-oxoisoindolin-4-yl)thio)-3,6,9,12,15-pentaoxaheptadecanoyl)piperazin-1-yl)benzoyl)piperidin-4-yl)butyl)-3-(pyridin-3-yl)acrylamide;
(E)-N-(4-(1-(4-(1-(3-((2-(2,6-dioxopiperidin-3-yl)-1,3-dioxoisoindolin-4-yl)amino)propanoyl)piperidin-4-yl)benzoyl)piperidin-4-yl)butyl)-3-(pyridin-3-yl)acrylamide;
(E)-N-(4-(1-(4-(1-(4-((2-(2,6-dioxopiperidin-3-yl)-1,3-dioxoisoindolin-4-yl)amino)butanoyl)piperidin-4-yl)benzoyl)piperidin-4-yl)butyl)-3-(pyridin-3-yl)acrylamide;
(E)-N-(4-(1-(4-(1-(5-((2-(2,6-dioxopiperidin-3-yl)-1,3-dioxoisoindolin-4-yl)amino)pentanoyl)piperidin-4-yl)benzoyl)piperidin-4-yl)butyl)-3-(pyridin-3-yl)acrylamide;
(E)-N-(4-(1-(4-(1-(7-((2-(2,6-dioxopiperidin-3-yl)-1,3-dioxoisoindolin-4-yl)amino)heptanoyl)piperidin-4-yl)benzoyl)piperidin-4-yl)butyl)-3-(pyridin-3-yl)acrylamide;
(E)-N-(4-(1-(4-(1-(2-((2-(2,6-dioxopiperidin-3-yl)-1,3-dioxoisoindolin-4-yl)thio)acetyl)piperidin-4-yl)benzoyl)piperidin-4-yl)butyl)-3-(pyridin-3-yl)acrylamide;
(E)-N-(4-(1-(4-(1-(3-((2-(2,6-dioxopiperidin-3-yl)-1,3-dioxoisoindolin-4-yl)thio)propanoyl)piperidin-4-yl)benzoyl)piperidin-4-yl)butyl)-3-(pyridin-3-yl)acrylamide;
(E)-N-(4-(1-(4-(1-(5-((2-(2,6-dioxopiperidin-3-yl)-1,3-dioxoisoindolin-4-yl)thio)pentanoyl)piperidin-4-yl)benzoyl)piperidin-4-yl)butyl)-3-(pyridin-3-yl)acrylamide;
(E)-N-(4-(1-(4-(1-(7-((2-(2,6-dioxopiperidin-3-yl)-1,3-dioxoisoindolin-4-yl)thio)heptanoyl)piperidin-4-yl)benzoyl)piperidin-4-yl)butyl)-3-(pyridin-3-yl)acrylamide;
(E)-N-(4-(1-(4-(1-(3-(2-((2-(2,6-dioxopiperidin-3-yl)-1,3-dioxoisoindolin-4-yl)thio)ethoxy)propanoyl)piperidin-4-yl)benzoyl)piperidin-4-yl)butyl)-3-(pyridin-3-yl)acrylamide;
(E)-N-(4-(1-(4-(1-(2-(2-((2-(2,6-dioxopiperidin-3-yl)-1,3-dioxoisoindolin-4-yl)thio)ethoxy)acetyl)piperidin-4-yl)benzoyl)piperidin-4-yl)butyl)-3-(pyridin-3-yl)acrylamide;
(E)-N-(4-(1-(4-(1-(3-(2-(2-((2-(2,6-dioxopiperidin-3-yl)-1,3-dioxoisoindolin-4-yl)thio)ethoxy)ethoxy)propanoyl)piperidin-4-yl)benzoyl)piperidin-4-yl)butyl)-3-(pyridin-3-yl)acrylamide;
(E)-N-(4-(1-(4-(1-(2-(2-(2-((2-(2,6-dioxopiperidin-3-yl)-1,3-dioxoisoindolin-4-yl)thio)ethoxy)ethoxy)acetyl)piperidin-4-yl)benzoyl)piperidin-4-yl)butyl)-3-(pyridin-3-yl)acrylamide;
(E)-N-(4-(1-(4-(1-(3-(2-(2-(2-((2-(2,6-dioxopiperidin-3-yl)-1,3-dioxoisoindolin-4-yl)thio)ethoxy)ethoxy)ethoxy)propanoyl)piperidin-4-yl)benzoyl)piperidin-4-yl)butyl)-3-(pyridin-3-yl)acrylamide;
(E)-N-(4-(1-(4-(1-(2-(2-(2-(2-((2-(2,6-dioxopiperidin-3-yl)-1,3-dioxoisoindolin-4-yl)thio)ethoxy)ethoxy)ethoxy)acetyl)piperidin-4-yl)benzoyl)piperidin-4-yl)butyl)-3-(pyridin-3-yl)acrylamide;
(E)-N-(4-(1-(4-(1-(17-((2-(2,6-dioxopiperidin-3-yl)-1,3-dioxoisoindolin-4-yl)thio)-3,6,9,12,15-pentaoxaheptadecanoyl)piperidin-4-yl)benzoyl)piperidin-4-yl)butyl)-3-(pyridin-3-yl)acrylamide;
(E)-N-(4-(1-(4-(1-(3-(2-((2-(2,6-dioxopiperidin-3-yl)-1,3-dioxoisoindolin-4-yl)amino)ethoxy)propanoyl)piperidin-4-yl)benzoyl)piperidin-4-yl)butyl)-3-(pyridin-3-yl)acrylamide;
(E)-N-(4-(1-(4-(1-(3-(2-(2-((2-(2,6-dioxopiperidin-3-yl)-1,3-dioxoisoindolin-4-yl)amino)ethoxy)ethoxy)propanoyl)piperidin-4-yl)benzoyl)piperidin-4-yl)butyl)-3-(pyridin-3-yl)acrylamide;
(E)-N-(4-(1-(4-(1-(2-((2-(2,6-dioxopiperidin-3-yl)-1,3-dioxoisoindolin-4-yl)oxy)acetyl)piperidin-4-yl)benzoyl)piperidin-4-yl)butyl)-3-(pyridin-3-yl)acrylamide;
(E)-N-(4-(1-(4-(1-(5-((2-(2,6-dioxopiperidin-3-yl)-1,3-dioxoisoindolin-4-yl)oxy)pentanoyl)piperidin-4-yl)benzoyl)piperidin-4-yl)butyl)-3-(pyridin-3-yl)acrylamide;
(E)-N-(4-(1-(4-(1-(7-((2-(2,6-dioxopiperidin-3-yl)-1,3-dioxoisoindolin-4-yl)oxy)heptanoyl)piperidin-4-yl)benzoyl)piperidin-4-yl)butyl)-3-(pyridin-3-yl)acrylamide;
(E)-N-(4-(1-(4-(1-((2-(2,6-dioxopiperidin-3-yl)-1-oxoisoindolin-4-yl)glycyl)piperidin-4-yl)benzoyl)piperidin-4-yl)butyl)-3-(pyridin-3-yl)acrylamide;
(E)-N-(4-(1-(4-(1-(4-((2-(2,6-dioxopiperidin-3-yl)-1-oxoisoindolin-4-yl)amino)butanoyl)piperidin-4-yl)benzoyl)piperidin-4-yl)butyl)-3-(pyridin-3-yl)acrylamide;
(E)-N-(4-(1-(4-(1-(5-((2-(2,6-dioxopiperidin-3-yl)-1-oxoisoindolin-4-yl)amino)pentanoyl)piperidin-4-yl)benzoyl)piperidin-4-yl)butyl)-3-(pyridin-3-yl)acrylamide;
(E)-N-(4-(1-(4-(1-(7-((2-(2,6-dioxopiperidin-3-yl)-1-oxoisoindolin-4-yl)amino)heptanoyl)piperidin-4-yl)benzoyl)piperidin-4-yl)butyl)-3-(pyridin-3-yl)acrylamide;
(E)-N-(4-(1-(4-(1-(2-(2-((2-(2,6-dioxopiperidin-3-yl)-1-oxoisoindolin-4-yl)amino)ethoxy)acetyl)piperidin-4-yl)benzoyl)piperidin-4-yl)butyl)-3-(pyridin-3-yl)acrylamide;
(E)-N-(4-(1-(4-(1-(3-(2-((2-(2,6-dioxopiperidin-3-yl)-1-oxoisoindolin-4-yl)amino)ethoxy)propanoyl)piperidin-4-yl)benzoyl)piperidin-4-yl)butyl)-3-(pyridin-3-yl)acrylamide;
(E)-N-(4-(1-(4-(1-(2-(2-(2-((2-(2,6-dioxopiperidin-3-yl)-1-oxoisoindolin-4-yl)amino)ethoxy)ethoxy)acetyl)piperidin-4-yl)benzoyl)piperidin-4-yl)butyl)-3-(pyridin-3-yl)acrylamide;
(E)-N-(4-(1-(4-(1-(3-(2-(2-((2-(2,6-dioxopiperidin-3-yl)-1-oxoisoindolin-4-yl)amino)ethoxy)ethoxy)propanoyl)piperidin-4-yl)benzoyl)piperidin-4-yl)butyl)-3-(pyridin-3-yl)acrylamide;
(E)-N-(4-(1-(4-(1-(2-(2-(2-(2-((2-(2,6-dioxopiperidin-3-yl)-1-oxoisoindolin-4-yl)amino)ethoxy)ethoxy)ethoxy)acetyl)piperidin-4-yl)benzoyl)piperidin-4-yl)butyl)-3-(pyridin-3-yl)acrylamide;
(E)-N-(4-(1-(4-(1-(3-(2-(2-(2-((2-(2,6-dioxopiperidin-3-yl)-1-oxoisoindolin-4-yl)amino)ethoxy)ethoxy)ethoxy)propanoyl)piperidin-4-yl)benzoyl)piperidin-4-yl)butyl)-3-(pyridin-3-yl)acrylamide;
(E)-N-(4-(1-(4-(1-(14-((2-(2,6-dioxopiperidin-3-yl)-1-oxoisoindolin-4-yl)amino)-3,6,9,12-tetraoxatetradecanoyl)piperidin-4-yl)benzoyl)piperidin-4-yl)butyl)-3-(pyridin-3-yl)acrylamide;
(E)-N-(4-(1-(4-(1-(1-((2-(2,6-dioxopiperidin-3-yl)-1-oxoisoindolin-4-yl)amino)-3,6,9,12-tetraoxapentadecan-15-oyl)piperidin-4-yl)benzoyl)piperidin-4-yl)butyl)-3-(pyridin-3-yl)acrylamide;
(E)-N-(4-(1-(4-(1-(2-((2-(2,6-dioxopiperidin-3-yl)-1-oxoisoindolin-4-yl)thio)acetyl)piperidin-4-yl)benzoyl)piperidin-4-yl)butyl)-3-(pyridin-3-yl)acrylamide;
(E)-N-(4-(1-(4-(1-(5-((2-(2,6-dioxopiperidin-3-yl)-1-oxoisoindolin-4-yl)thio)pentanoyl)piperidin-4-yl)benzoyl)piperidin-4-yl)butyl)-3-(pyridin-3-yl)acrylamide;
(E)-N-(4-(1-(4-(1-(6-((2-(2,6-dioxopiperidin-3-yl)-1-oxoisoindolin-4-yl)thio)hexanoyl)piperidin-4-yl)benzoyl)piperidin-4-yl)butyl)-3-(pyridin-3-yl)acrylamide;
(E)-N-(4-(1-(4-(1-(2-(2-((2-(2,6-dioxopiperidin-3-yl)-1-oxoisoindolin-4-yl)thio)ethoxy)acetyl)piperidin-4-yl)benzoyl)piperidin-4-yl)butyl)-3-(pyridin-3-yl)acrylamide;
(E)-N-(4-(1-(4-(1-(2-(2-(2-((2-(2,6-dioxopiperidin-3-yl)-1-oxoisoindolin-4-yl)thio)ethoxy)ethoxy)acetyl)piperidin-4-yl)benzoyl)piperidin-4-yl)butyl)-3-(pyridin-3-yl)acrylamide;
(E)-N-(4-(1-(4-(1-(17-((2-(2,6-dioxopiperidin-3-yl)-1-oxoisoindolin-4-yl)thio)-3,6,9,12,15-pentaoxaheptadecanoyl)piperidin-4-yl)benzoyl)piperidin-4-yl)butyl)-3-(pyridin-3-yl)acrylamide;
(E)-N-(4-(1-(4-(1-(5-(2-(2,6-dioxopiperidin-3-yl)-1-oxoisoindolin-4-yl)pent-4-yn-1-yl)piperidin-4-yl)benzoyl)piperidin-4-yl)butyl)-3-(pyridin-3-yl)acrylamide;
(E)-N-(4-(1-(4-(1-(6-(2-(2,6-dioxopiperidin-3-yl)-1-oxoisoindolin-4-yl)hex-5-yn-1-yl)piperidin-4-yl)benzoyl)piperidin-4-yl)butyl)-3-(pyridin-3-yl)acrylamide;
(E)-N-(4-(1-(4-(1-(9-(2-(2,6-dioxopiperidin-3-yl)-1-oxoisoindolin-4-yl)non-8-yn-1-yl)piperidin-4-yl)benzoyl)piperidin-4-yl)butyl)-3-(pyridin-3-yl)acrylamide;
(E)-N-(4-(1-(4-(1-(5-(2-(2,6-dioxopiperidin-3-yl)-1,3-dioxoisoindolin-4-yl)pent-4-yn-1-yl)piperidin-4-yl)benzoyl)piperidin-4-yl)butyl)-3-(pyridin-3-yl)acrylamide;
(E)-N-(4-(1-(4-(1-(6-(2-(2,6-dioxopiperidin-3-yl)-1,3-dioxoisoindolin-4-yl)hex-5-yn-1-yl)piperidin-4-yl)benzoyl)piperidin-4-yl)butyl)-3-(pyridin-3-yl)acrylamide;
(E)-N-(4-(1-(4-(1-(7-(2-(2,6-dioxopiperidin-3-yl)-1,3-dioxoisoindolin-4-yl)hept-6-yn-1-yl)piperidin-4-yl)benzoyl)piperidin-4-yl)butyl)-3-(pyridin-3-yl)acrylamide;
(E)-N-(4-(1-(4-(1-(2-((2-(2,6-dioxopiperidin-3-yl)-1-oxoisoindolin-4-yl)thio)ethyl)piperidin-4-yl)benzoyl)piperidin-4-yl)butyl)-3-(pyridin-3-yl)acrylamide;
(E)-N-(4-(1-(4-(1-(3-((2-(2,6-dioxopiperidin-3-yl)-1-oxoisoindolin-4-yl)thio)propyl)piperidin-4-yl)benzoyl)piperidin-4-yl)butyl)-3-(pyridin-3-yl)acrylamide;
(E)-N-(4-(1-(4-(1-(5-((2-(2,6-dioxopiperidin-3-yl)-1-oxoisoindolin-4-yl)thio)pentyl)piperidin-4-yl)benzoyl)piperidin-4-yl)butyl)-3-(pyridin-3-yl)acrylamide;
(E)-N-(4-(1-(4-(1-(6-((2-(2,6-dioxopiperidin-3-yl)-1-oxoisoindolin-4-yl)thio)hexyl)piperidin-4-yl)benzoyl)piperidin-4-yl)butyl)-3-(pyridin-3-yl)acrylamide;
(E)-N-(4-(1-(4-(1-(2-((2-(2,6-dioxopiperidin-3-yl)-1,3-dioxoisoindolin-4-yl)thio)ethyl)piperidin-4-yl)benzoyl)piperidin-4-yl)butyl)-3-(pyridin-3-yl)acrylamide;
(E)-N-(4-(1-(4-(1-(3-((2-(2,6-dioxopiperidin-3-yl)-1,3-dioxoisoindolin-4-yl)thio)propyl)piperidin-4-yl)benzoyl)piperidin-4-yl)butyl)-3-(pyridin-3-yl)acrylamide;
(E)-N-(4-(1-(4-(1-(4-((2-(2,6-dioxopiperidin-3-yl)-1,3-dioxoisoindolin-4-yl)thio)butyl)piperidin-4-yl)benzoyl)piperidin-4-yl)butyl)-3-(pyridin-3-yl)acrylamide;
(E)-N-(4-(1-(4-(1-(5-((2-(2,6-dioxopiperidin-3-yl)-1,3-dioxoisoindolin-4-yl)thio)pentyl)piperidin-4-yl)benzoyl)piperidin-4-yl)butyl)-3-(pyridin-3-yl)acrylamide;
(E)-N-(4-(1-(4-(1-(6-((2-(2,6-dioxopiperidin-3-yl)-1,3-dioxoisoindolin-4-yl)thio)hexyl)piperidin-4-yl)benzoyl)piperidin-4-yl)butyl)-3-(pyridin-3-yl)acrylamide;
(E)-N-(4-(1-(4-(1-(8-((2-(2,6-dioxopiperidin-3-yl)-1,3-dioxoisoindolin-4-yl)thio)octyl)piperidin-4-yl)benzoyl)piperidin-4-yl)butyl)-3-(pyridin-3-yl)acrylamide;
(E)-N-(4-(1-(4-(1-(5-((2-(2,6-dioxopiperidin-3-yl)-1-oxoisoindolin-4-yl)oxy)pentyl)piperidin-4-yl)benzoyl)piperidin-4-yl)butyl)-3-(pyridin-3-yl)acrylamide;
(E)-N-(4-(1-(4-(1-(6-((2-(2,6-dioxopiperidin-3-yl)-1-oxoisoindolin-4-yl)oxy)hexyl)piperidin-4-yl)benzoyl)piperidin-4-yl)butyl)-3-(pyridin-3-yl)acrylamide;
(E)-N-(4-(1-(4-(1-(3-((2-(2,6-dioxopiperidin-3-yl)-1,3-dioxoisoindolin-4-yl)oxy)propyl)piperidin-4-yl)benzoyl)piperidin-4-yl)butyl)-3-(pyridin-3-yl)acrylamide;
(E)-N-(4-(1-(4-(1-(5-((2-(2,6-dioxopiperidin-3-yl)-1,3-dioxoisoindolin-4-yl)oxy)pentyl)piperidin-4-yl)benzoyl)piperidin-4-yl)butyl)-3-(pyridin-3-yl)acrylamide;
(2S,4R)-1-((S)-3,3-dimethyl-2-(3-oxo-3-(4-(4-(4-(4-((E)-3-(pyridin-3-yl)acrylamido)butyl)piperidine-1-carbonyl)phenyl)piperidin-1-yl)propanamido)butanoyl)-4-hydroxy-N-(4-(4-methylthiazol-5-yl)benzyl)pyrrolidine-2-carboxamide;
(2S,4R)-1-((S)-3,3-dimethyl-2-(5-oxo-5-(4-(4-(4-(4-((E)-3-(pyridin-3-yl)acrylamido)butyl)piperidine-1-carbonyl)phenyl)piperidin-1-yl)pentanamido)butanoyl)-4-hydroxy-N-(4-(4-methylthiazol-5-yl)benzyl)pyrrolidine-2-carboxamide;
(2S,4R)-1-((S)-3,3-dimethyl-2-(7-oxo-7-(4-(4-(4-(4-((E)-3-(pyridin-3-yl)acrylamido)butyl)piperidine-1-carbonyl)phenyl)piperidin-1-yl)heptanamido)butanoyl)-4-hydroxy-N-(4-(4-methylthiazol-5-yl)benzyl)pyrrolidine-2-carboxamide;
(2S,4R)-1-((S)-3,3-dimethyl-2-(10-oxo-10-(4-(4-(4-(4-((E)-3-(pyridin-3-yl)acrylamido)butyl)piperidine-1-carbonyl)phenyl)piperidin-1-yl)decanamido)butanoyl)-4-hydroxy-N-(4-(4-methylthiazol-5-yl)benzyl)pyrrolidine-2-carboxamide;
(2S,4R)-1-((S)-3,3-dimethyl-2-(2-(2-(2-oxo-2-(4-(4-(4-(4-((E)-3-(pyridin-3-yl)acrylamido)butyl)piperidine-1-carbonyl)phenyl)piperidin-1-yl)ethoxy)ethoxy)acetamido)butanoyl)-4-hydroxy-N-(4-(4-methylthiazol-5-yl)benzyl)pyrrolidine-2-carboxamide;
(2S,4R)-1-((S)-3,3-dimethyl-2-(3-(2-(3-oxo-3-(4-(4-(4-(4-((E)-3-(pyridin-3-yl)acrylamido)butyl)piperidine-1-carbonyl)phenyl)piperidin-1-yl)propoxy)ethoxy)propanamido)butanoyl)-4-hydroxy-N-(4-(4-methylthiazol-5-yl)benzyl)pyrrolidine-2-carboxamide;
(2S,4R)-1-((S)-2-(tert-butyl)-4,16-dioxo-16-(4-(4-(4-(4-((E)-3-(pyridin-3-yl)acrylamido)butyl)piperidine-1-carbonyl)phenyl)piperidin-1-yl)-7,10,13-trioxa-3-azahexadecanoyl)-4-hydroxy-N-(4-(4-methylthiazol-5-yl)benzyl)pyrrolidine-2-carboxamide;
(2S,4R)-1-((S)-2-(tert-butyl)-4,22-dioxo-22-(4-(4-(4-(4-((E)-3-(pyridin-3-yl)acrylamido)butyl)piperidine-1-carbonyl)phenyl)piperidin-1-yl)-7,10,13,16,19-pentaoxa-3-azadocosanoyl)-4-hydroxy-N-(4-(4-methylthiazol-5-yl)benzyl)pyrrolidine-2-carboxamide;
(E)-N-(4-(1-(6-(4-(3-((2-(2,6-dioxopiperidin-3-yl)-1,3-dioxoisoindolin-4-yl)amino)propanoyl)piperazin-1-yl)nicotinoyl)piperidin-4-yl)butyl)-3-(pyridin-3-yl)acrylamide;
(E)-N-(4-(1-(6-(4-(5-((2-(2,6-dioxopiperidin-3-yl)-1,3-dioxoisoindolin-4-yl)amino)pentanoyl)piperazin-1-yl)nicotinoyl)piperidin-4-yl)butyl)-3-(pyridin-3-yl)acrylamide;
(E)-N-(4-(1-(6-(4-(7-((2-(2,6-dioxopiperidin-3-yl)-1,3-dioxoisoindolin-4-yl)amino)heptanoyl)piperazin-1-yl)nicotinoyl)piperidin-4-yl)butyl)-3-(pyridin-3-yl)acrylamide;
(E)-N-(4-(1-(6-(4-(3-(2-((2-(2,6-dioxopiperidin-3-yl)-1,3-dioxoisoindolin-4-yl)amino)ethoxy)propanoyl)piperazin-1-yl)nicotinoyl)piperidin-4-yl)butyl)-3-(pyridin-3-yl)acrylamide;
(E)-N-(4-(1-(6-(4-(3-(2-(2-((2-(2,6-dioxopiperidin-3-yl)-1,3-dioxoisoindolin-4-yl)amino)ethoxy)ethoxy)propanoyl)piperazin-1-yl)nicotinoyl)piperidin-4-yl)butyl)-3-(pyridin-3-yl)acrylamide;
(E)-N-(4-(1-(6-(4-(2-((2-(2,6-dioxopiperidin-3-yl)-1,3-dioxoisoindolin-4-yl)thio)acetyl)piperazin-1-yl)nicotinoyl)piperidin-4-yl)butyl)-3-(pyridin-3-yl)acrylamide;
(E)-N-(4-(1-(6-(4-(3-((2-(2,6-dioxopiperidin-3-yl)-1,3-dioxoisoindolin-4-yl)thio)propanoyl)piperazin-1-yl)nicotinoyl)piperidin-4-yl)butyl)-3-(pyridin-3-yl)acrylamide;
(E)-N-(4-(1-(6-(4-(6-((2-(2,6-dioxopiperidin-3-yl)-1,3-dioxoisoindolin-4-yl)thio)hexanoyl)piperazin-1-yl)nicotinoyl)piperidin-4-yl)butyl)-3-(pyridin-3-yl)acrylamide;
(E)-N-(4-(1-(6-(4-(7-((2-(2,6-dioxopiperidin-3-yl)-1,3-dioxoisoindolin-4-yl)thio)heptanoyl)piperazin-1-yl)nicotinoyl)piperidin-4-yl)butyl)-3-(pyridin-3-yl)acrylamide;
(E)-N-(4-(1-(6-(4-(2-(2-((2-(2,6-dioxopiperidin-3-yl)-1,3-dioxoisoindolin-4-yl)thio)ethoxy)acetyl)piperazin-1-yl)nicotinoyl)piperidin-4-yl)butyl)-3-(pyridin-3-yl)acrylamide;
(E)-N-(4-(1-(6-(4-(3-(2-((2-(2,6-dioxopiperidin-3-yl)-1,3-dioxoisoindolin-4-yl)thio)ethoxy)propanoyl)piperazin-1-yl)nicotinoyl)piperidin-4-yl)butyl)-3-(pyridin-3-yl)acrylamide;
(E)-N-(4-(1-(6-(4-(2-(2-(2-(2-((2-(2,6-dioxopiperidin-3-yl)-1,3-dioxoisoindolin-4-yl)thio)ethoxy)ethoxy)ethoxy)acetyl)piperazin-1-yl)nicotinoyl)piperidin-4-yl)butyl)-3-(pyridin-3-yl)acrylamide;
(E)-N-(4-(1-(6-(4-(3-(2-(2-(2-((2-(2,6-dioxopiperidin-3-yl)-1,3-dioxoisoindolin-4-yl)thio)ethoxy)ethoxy)ethoxy)propanoyl)piperazin-1-yl)nicotinoyl)piperidin-4-yl)butyl)-3-(pyridin-3-yl)acrylamide;
(E)-N-(4-(1-(6-(4-(14-((2-(2,6-dioxopiperidin-3-yl)-1,3-dioxoisoindolin-4-yl)thio)-3,6,9,12-tetraoxatetradecanoyl)piperazin-1-yl)nicotinoyl)piperidin-4-yl)butyl)-3-(pyridin-3-yl)acrylamide;
(E)-N-(4-(1-(6-(4-(17-((2-(2,6-dioxopiperidin-3-yl)-1,3-dioxoisoindolin-4-yl)thio)-3,6,9,12,15-pentaoxaheptadecanoyl)piperazin-1-yl)nicotinoyl)piperidin-4-yl)butyl)-3-(pyridin-3-yl)acrylamide;
(E)-N-(4-(1-(6-(4-(2-((2-(2,6-dioxopiperidin-3-yl)-1,3-dioxoisoindolin-4-yl)oxy)acetyl)piperazin-1-yl)nicotinoyl)piperidin-4-yl)butyl)-3-(pyridin-3-yl)acrylamide;
(E)-N-(4-(1-(6-(4-(5-((2-(2,6-dioxopiperidin-3-yl)-1,3-dioxoisoindolin-4-yl)oxy)pentanoyl)piperazin-1-yl)nicotinoyl)piperidin-4-yl)butyl)-3-(pyridin-3-yl)acrylamide;
(E)-N-(4-(1-(6-(4-(7-((2-(2,6-dioxopiperidin-3-yl)-1,3-dioxoisoindolin-4-yl)oxy)heptanoyl)piperazin-1-yl)nicotinoyl)piperidin-4-yl)butyl)-3-(pyridin-3-yl)acrylamide;
(E)-N-(4-(1-(6-(4-(5-(2-(2,6-dioxopiperidin-3-yl)-1,3-dioxoisoindolin-4-yl)pent-4-yn-1-yl)piperazin-1-yl)nicotinoyl)piperidin-4-yl)butyl)-3-(pyridin-3-yl)acrylamide;
(E)-N-(4-(1-(6-(4-(6-(2-(2,6-dioxopiperidin-3-yl)-1,3-dioxoisoindolin-4-yl)hex-5-yn-1-yl)piperazin-1-yl)nicotinoyl)piperidin-4-yl)butyl)-3-(pyridin-3-yl)acrylamide;
(E)-N-(4-(1-(6-(4-(7-(2-(2,6-dioxopiperidin-3-yl)-1,3-dioxoisoindolin-4-yl)hept-6-yn-1-yl)piperazin-1-yl)nicotinoyl)piperidin-4-yl)butyl)-3-(pyridin-3-yl)acrylamide;
(E)-N-(4-(1-(6-(4-(2-((2-(2,6-dioxopiperidin-3-yl)-1,3-dioxoisoindolin-4-yl)thio)ethyl)piperazin-1-yl)nicotinoyl)piperidin-4-yl)butyl)-3-(pyridin-3-yl)acrylamide;
(E)-N-(4-(1-(6-(4-(3-((2-(2,6-dioxopiperidin-3-yl)-1,3-dioxoisoindolin-4-yl)thio)propyl)piperazin-1-yl)nicotinoyl)piperidin-4-yl)butyl)-3-(pyridin-3-yl)acrylamide;
(E)-N-(4-(1-(6-(4-(5-((2-(2,6-dioxopiperidin-3-yl)-1,3-dioxoisoindolin-4-yl)thio)pentyl)piperazin-1-yl)nicotinoyl)piperidin-4-yl)butyl)-3-(pyridin-3-yl)acrylamide;
(E)-N-(4-(1-(6-(4-(6-((2-(2,6-dioxopiperidin-3-yl)-1,3-dioxoisoindolin-4-yl)thio)hexyl)piperazin-1-yl)nicotinoyl)piperidin-4-yl)butyl)-3-(pyridin-3-yl)acrylamide;
(E)-N-(4-(1-(6-(4-(4-((2-(2,6-dioxopiperidin-3-yl)-1,3-dioxoisoindolin-4-yl)oxy)butyl)piperazin-1-yl)nicotinoyl)piperidin-4-yl)butyl)-3-(pyridin-3-yl)acrylamide;
(E)-N-(4-(1-(6-(4-(8-((2-(2,6-dioxopiperidin-3-yl)-1,3-dioxoisoindolin-4-yl)oxy)octyl)piperazin-1-yl)nicotinoyl)piperidin-4-yl)butyl)-3-(pyridin-3-yl)acrylamide;
(E)-N-(4-(1-(6-(4-(6-(2-(2,6-dioxopiperidin-3-yl)-1,3-dioxoisoindolin-4-yl)hexyl)piperazin-1-yl)nicotinoyl)piperidin-4-yl)butyl)-3-(pyridin-3-yl)acrylamide;
(E)-N-(4-(1-(6-(4-(7-(2-(2,6-dioxopiperidin-3-yl)-1,3-dioxoisoindolin-4-yl)heptyl)piperazin-1-yl)nicotinoyl)piperidin-4-yl)butyl)-3-(pyridin-3-yl)acrylamide;
(E)-N-(4-(1-(6-(4-(5-(2-(2,6-dioxopiperidin-3-yl)-1,3-dioxoisoindolin-4-yl)pentyl)piperazin-1-yl)nicotinoyl)piperidin-4-yl)butyl)-3-(pyridin-3-yl)acrylamide;
(E)-N-(4-(1-(6-(4-((2-(2,6-dioxopiperidin-3-yl)-1-oxoisoindolin-4-yl)glycyl)piperazin-1-yl)nicotinoyl)piperidin-4-yl)butyl)-3-(pyridin-3-yl)acrylamide;
(E)-N-(4-(1-(6-(4-(4-((2-(2,6-dioxopiperidin-3-yl)-1-oxoisoindolin-4-yl)amino)butanoyl)piperazin-1-yl)nicotinoyl)piperidin-4-yl)butyl)-3-(pyridin-3-yl)acrylamide;
(E)-N-(4-(1-(6-(4-(6-((2-(2,6-dioxopiperidin-3-yl)-1-oxoisoindolin-4-yl)amino)hexanoyl)piperazin-1-yl)nicotinoyl)piperidin-4-yl)butyl)-3-(pyridin-3-yl)acrylamide;
(E)-N-(4-(1-(6-(4-(2-(2-((2-(2,6-dioxopiperidin-3-yl)-1-oxoisoindolin-4-yl)amino)ethoxy)acetyl)piperazin-1-yl)nicotinoyl)piperidin-4-yl)butyl)-3-(pyridin-3-yl)acrylamide;
(E)-N-(4-(1-(6-(4-(3-(2-((2-(2,6-dioxopiperidin-3-yl)-1-oxoisoindolin-4-yl)amino)ethoxy)propanoyl)piperazin-1-yl)nicotinoyl)piperidin-4-yl)butyl)-3-(pyridin-3-yl)acrylamide;
(E)-N-(4-(1-(6-(4-(2-(2-(2-((2-(2,6-dioxopiperidin-3-yl)-1-oxoisoindolin-4-yl)amino)ethoxy)ethoxy)acetyl)piperazin-1-yl)nicotinoyl)piperidin-4-yl)butyl)-3-(pyridin-3-yl)acrylamide;
(E)-N-(4-(1-(6-(4-(3-(2-(2-((2-(2,6-dioxopiperidin-3-yl)-1-oxoisoindolin-4-yl)amino)ethoxy)ethoxy)propanoyl)piperazin-1-yl)nicotinoyl)piperidin-4-yl)butyl)-3-(pyridin-3-yl)acrylamide;
(E)-N-(4-(1-(6-(4-(14-((2-(2,6-dioxopiperidin-3-yl)-1-oxoisoindolin-4-yl)amino)-3,6,9,12-tetraoxatetradecanoyl)piperazin-1-yl)nicotinoyl)piperidin-4-yl)butyl)-3-(pyridin-3-yl)acrylamide;
(E)-N-(4-(1-(6-(4-(1-((2-(2,6-dioxopiperidin-3-yl)-1-oxoisoindolin-4-yl)amino)-3,6,9,12-tetraoxapentadecan-15-oyl)piperazin-1-yl)nicotinoyl)piperidin-4-yl)butyl)-3-(pyridin-3-yl)acrylamide;
(E)-N-(4-(1-(6-(4-(5-((2-(2,6-dioxopiperidin-3-yl)-1-oxoisoindolin-4-yl)thio)pentanoyl)piperazin-1-yl)nicotinoyl)piperidin-4-yl)butyl)-3-(pyridin-3-yl)acrylamide;
(E)-N-(4-(1-(6-(4-(6-((2-(2,6-dioxopiperidin-3-yl)-1-oxoisoindolin-4-yl)thio)hexanoyl)piperazin-1-yl)nicotinoyl)piperidin-4-yl)butyl)-3-(pyridin-3-yl)acrylamide;
(E)-N-(4-(1-(6-(4-(2-(2-((2-(2,6-dioxopiperidin-3-yl)-1-oxoisoindolin-4-yl)thio)ethoxy)acetyl)piperazin-1-yl)nicotinoyl)piperidin-4-yl)butyl)-3-(pyridin-3-yl)acrylamide;
(E)-N-(4-(1-(6-(4-(2-(2-(2-((2-(2,6-dioxopiperidin-3-yl)-1-oxoisoindolin-4-yl)thio)ethoxy)ethoxy)acetyl)piperazin-1-yl)nicotinoyl)piperidin-4-yl)butyl)-3-(pyridin-3-yl)acrylamide;
(E)-N-(4-(1-(6-(4-(17-((2-(2,6-dioxopiperidin-3-yl)-1-oxoisoindolin-4-yl)thio)-3,6,9,12,15-pentaoxaheptadecanoyl)piperazin-1-yl)nicotinoyl)piperidin-4-yl)butyl)-3-(pyridin-3-yl)acrylamide;
(E)-N-(4-(1-(6-(4-(2-((2-(2,6-dioxopiperidin-3-yl)-1-oxoisoindolin-4-yl)oxy)acetyl)piperazin-1-yl)nicotinoyl)piperidin-4-yl)butyl)-3-(pyridin-3-yl)acrylamide;
(E)-N-(4-(1-(6-(4-(5-((2-(2,6-dioxopiperidin-3-yl)-1-oxoisoindolin-4-yl)oxy)pentanoyl)piperazin-1-yl)nicotinoyl)piperidin-4-yl)butyl)-3-(pyridin-3-yl)acrylamide;
(E)-N-(4-(1-(6-(4-(7-((2-(2,6-dioxopiperidin-3-yl)-1-oxoisoindolin-4-yl)oxy)heptanoyl)piperazin-1-yl)nicotinoyl)piperidin-4-yl)butyl)-3-(pyridin-3-yl)acrylamide;
(E)-N-(4-(1-(6-(4-(5-(2-(2,6-dioxopiperidin-3-yl)-1-oxoisoindolin-4-yl)pent-4-yn-1-yl)piperazin-1-yl)nicotinoyl)piperidin-4-yl)butyl)-3-(pyridin-3-yl)acrylamide;
(E)-N-(4-(1-(6-(4-(6-(2-(2,6-dioxopiperidin-3-yl)-1-oxoisoindolin-4-yl)hex-5-yn-1-yl)piperazin-1-yl)nicotinoyl)piperidin-4-yl)butyl)-3-(pyridin-3-yl)acrylamide;
(E)-N-(4-(1-(6-(4-(9-(2-(2,6-dioxopiperidin-3-yl)-1-oxoisoindolin-4-yl)non-8-yn-1-yl)piperazin-1-yl)nicotinoyl)piperidin-4-yl)butyl)-3-(pyridin-3-yl)acrylamide;
(E)-N-(4-(1-(6-(4-(2-((2-(2,6-dioxopiperidin-3-yl)-1-oxoisoindolin-4-yl)thio)ethyl)piperazin-1-yl)nicotinoyl)piperidin-4-yl)butyl)-3-(pyridin-3-yl)acrylamide;
(E)-N-(4-(1-(6-(4-(3-((2-(2,6-dioxopiperidin-3-yl)-1-oxoisoindolin-4-yl)thio)propyl)piperazin-1-yl)nicotinoyl)piperidin-4-yl)butyl)-3-(pyridin-3-yl)acrylamide;
(E)-N-(4-(1-(6-(4-(5-((2-(2,6-dioxopiperidin-3-yl)-1-oxoisoindolin-4-yl)thio)pentyl)piperazin-1-yl)nicotinoyl)piperidin-4-yl)butyl)-3-(pyridin-3-yl)acrylamide;
(E)-N-(4-(1-(6-(4-(8-((2-(2,6-dioxopiperidin-3-yl)-1-oxoisoindolin-4-yl)thio)octyl)piperazin-1-yl)nicotinoyl)piperidin-4-yl)butyl)-3-(pyridin-3-yl)acrylamide;
(E)-N-(4-(1-(6-(4-(4-((2-(2,6-dioxopiperidin-3-yl)-1-oxoisoindolin-4-yl)oxy)butyl)piperazin-1-yl)nicotinoyl)piperidin-4-yl)butyl)-3-(pyridin-3-yl)acrylamide;
(E)-N-(4-(1-(6-(4-(8-((2-(2,6-dioxopiperidin-3-yl)-1-oxoisoindolin-4-yl)oxy)octyl)piperazin-1-yl)nicotinoyl)piperidin-4-yl)butyl)-3-(pyridin-3-yl)acrylamide;
(E)-N-(2,6-dioxopiperidin-3-yl)-3-(6-(4-(5-(4-(4-(3-(pyridin-3-yl)acrylamido)butyl)piperidine-1-carbonyl)pyridin-2-yl)piperazin-1-yl)hexyl)benzamide;
(E)-N-(4-(1-(6-(4-(7-(2-(2,6-dioxopiperidin-3-yl)-1-oxoisoindolin-4-yl)heptyl)piperazin-1-yl)nicotinoyl)piperidin-4-yl)butyl)-3-(pyridin-3-yl)acrylamide;
(E)-N-(4-(1-(6-(4-(5-(2-(2,6-dioxopiperidin-3-yl)-1-oxoisoindolin-4-yl)pentyl)piperazin-1-yl)nicotinoyl)piperidin-4-yl)butyl)-3-(pyridin-3-yl)acrylamide;
(2S,4R)-1-((S)-3,3-dimethyl-2-(3-oxo-3-(4-(5-(4-(4-((E)-3-(pyridin-3-yl)acrylamido)butyl)piperidine-1-carbonyl)pyridin-2-yl)piperazin-1-yl)propanamido)butanoyl)-4-hydroxy-N-(4-(4-methylthiazol-5-yl)benzyl)pyrrolidine-2-carboxamide;
(2S,4R)-1-((S)-3,3-dimethyl-2-(5-oxo-5-(4-(5-(4-(4-((E)-3-(pyridin-3-yl)acrylamido)butyl)piperidine-1-carbonyl)pyridin-2-yl)piperazin-1-yl)pentanamido)butanoyl)-4-hydroxy-N-(4-(4-methylthiazol-5-yl)benzyl)pyrrolidine-2-carboxamide;
(2S,4R)-1-((S)-3,3-dimethyl-2-(7-oxo-7-(4-(5-(4-(4-((E)-3-(pyridin-3-yl)acrylamido)butyl)piperidine-1-carbonyl)pyridin-2-yl)piperazin-1-yl)heptanamido)butanoyl)-4-hydroxy-N-(4-(4-methylthiazol-5-yl)benzyl)pyrrolidine-2-carboxamide;
(2S,4R)-1-((S)-3,3-dimethyl-2-(3-(2-(3-oxo-3-(4-(5-(4-(4-((E)-3-(pyridin-3-yl)acrylamido)butyl)piperidine-1-carbonyl)pyridin-2-yl)piperazin-1-yl)propoxy)ethoxy)propanamido)butanoyl)-4-hydroxy-N-(4-(4-methylthiazol-5-yl)benzyl)pyrrolidine-2-carboxamide;
(2S,4R)-1-((S)-2-(tert-butyl)-4,19-dioxo-19-(4-(5-(4-(4-((E)-3-(pyridin-3-yl)acrylamido)butyl)piperidine-1-carbonyl)pyridin-2-yl)piperazin-1-yl)-7,10,13,16-tetraoxa-3-azanonadecanoyl)-4-hydroxy-N-(4-(4-methylthiazol-5-yl)benzyl)pyrrolidine-2-carboxamide;
(2S,4R)-1-((S)-2-(tert-butyl)-4,22-dioxo-22-(4-(5-(4-(4-((E)-3-(pyridin-3-yl)acrylamido)butyl)piperidine-1-carbonyl)pyridin-2-yl)piperazin-1-yl)-7,10,13,16,19-pentaoxa-3-azadocosanoyl)-4-hydroxy-N-(4-(4-methylthiazol-5-yl)benzyl)pyrrolidine-2-carboxamide;
(E)-N-(4-(1-(6-(4-(3-((2-(2,6-dioxopiperidin-3-yl)-1,3-dioxoisoindolin-4-yl)amino)propanoyl)piperazin-1-yl)pyridazine-3-carbonyl)piperidin-4-yl)butyl)-3-(pyridin-3-yl)acrylamide;
(E)-N-(4-(1-(6-(4-(5-((2-(2,6-dioxopiperidin-3-yl)-1,3-dioxoisoindolin-4-yl)amino)pentanoyl)piperazin-1-yl)pyridazine-3-carbonyl)piperidin-4-yl)butyl)-3-(pyridin-3-yl)acrylamide;
(E)-N-(4-(1-(6-(4-(7-((2-(2,6-dioxopiperidin-3-yl)-1,3-dioxoisoindolin-4-yl)amino)heptanoyl)piperazin-1-yl)pyridazine-3-carbonyl)piperidin-4-yl)butyl)-3-(pyridin-3-yl)acrylamide;
(E)-N-(4-(1-(6-(4-(3-(2-((2-(2,6-dioxopiperidin-3-yl)-1,3-dioxoisoindolin-4-yl)amino)ethoxy)propanoyl)piperazin-1-yl)pyridazine-3-carbonyl)piperidin-4-yl)butyl)-3-(pyridin-3-yl)acrylamide;
(E)-N-(4-(1-(6-(4-(3-(2-(2-((2-(2,6-dioxopiperidin-3-yl)-1,3-dioxoisoindolin-4-yl)amino)ethoxy)ethoxy)propanoyl)piperazin-1-yl)pyridazine-3-carbonyl)piperidin-4-yl)butyl)-3-(pyridin-3-yl)acrylamide;
(E)-N-(4-(1-(6-(4-(2-((2-(2,6-dioxopiperidin-3-yl)-1,3-dioxoisoindolin-4-yl)thio)acetyl)piperazin-1-yl)pyridazine-3-carbonyl)piperidin-4-yl)butyl)-3-(pyridin-3-yl)acrylamide;
(E)-N-(4-(1-(6-(4-(3-((2-(2,6-dioxopiperidin-3-yl)-1,3-dioxoisoindolin-4-yl)thio)propanoyl)piperazin-1-yl)pyridazine-3-carbonyl)piperidin-4-yl)butyl)-3-(pyridin-3-yl)acrylamide;
(E)-N-(4-(1-(6-(4-(6-((2-(2,6-dioxopiperidin-3-yl)-1,3-dioxoisoindolin-4-yl)thio)hexanoyl)piperazin-1-yl)pyridazine-3-carbonyl)piperidin-4-yl)butyl)-3-(pyridin-3-yl)acrylamide;
(E)-N-(4-(1-(6-(4-(7-((2-(2,6-dioxopiperidin-3-yl)-1,3-dioxoisoindolin-4-yl)thio)heptanoyl)piperazin-1-yl)pyridazine-3-carbonyl)piperidin-4-yl)butyl)-3-(pyridin-3-yl)acrylamide;
(E)-N-(4-(1-(6-(4-(2-(2-((2-(2,6-dioxopiperidin-3-yl)-1,3-dioxoisoindolin-4-yl)thio)ethoxy)acetyl)piperazin-1-yl)pyridazine-3-carbonyl)piperidin-4-yl)butyl)-3-(pyridin-3-yl)acrylamide;
(E)-N-(4-(1-(6-(4-(3-(2-((2-(2,6-dioxopiperidin-3-yl)-1,3-dioxoisoindolin-4-yl)thio)ethoxy)propanoyl)piperazin-1-yl)pyridazine-3-carbonyl)piperidin-4-yl)butyl)-3-(pyridin-3-yl)acrylamide;
(E)-N-(4-(1-(6-(4-(2-(2-(2-(2-((2-(2,6-dioxopiperidin-3-yl)-1,3-dioxoisoindolin-4-yl)thio)ethoxy)ethoxy)ethoxy)acetyl)piperazin-1-yl)pyridazine-3-carbonyl)piperidin-4-yl)butyl)-3-(pyridin-3-yl)acrylamide;
(E)-N-(4-(1-(6-(4-(3-(2-(2-(2-((2-(2,6-dioxopiperidin-3-yl)-1,3-dioxoisoindolin-4-yl)thio)ethoxy)ethoxy)ethoxy)propanoyl)piperazin-1-yl)pyridazine-3-carbonyl)piperidin-4-yl)butyl)-3-(pyridin-3-yl)acrylamide;
(E)-N-(4-(1-(6-(4-(14-((2-(2,6-dioxopiperidin-3-yl)-1,3-dioxoisoindolin-4-yl)thio)-3,6,9,12-tetraoxatetradecanoyl)piperazin-1-yl)pyridazine-3-carbonyl)piperidin-4-yl)butyl)-3-(pyridin-3-yl)acrylamide;
(E)-N-(4-(1-(6-(4-(17-((2-(2,6-dioxopiperidin-3-yl)-1,3-dioxoisoindolin-4-yl)thio)-3,6,9,12,15-pentaoxaheptadecanoyl)piperazin-1-yl)pyridazine-3-carbonyl)piperidin-4-yl)butyl)-3-(pyridin-3-yl)acrylamide;
(E)-N-(4-(1-(6-(4-(2-((2-(2,6-dioxopiperidin-3-yl)-1,3-dioxoisoindolin-4-yl)oxy)acetyl)piperazin-1-yl)pyridazine-3-carbonyl)piperidin-4-yl)butyl)-3-(pyridin-3-yl)acrylamide;
(E)-N-(4-(1-(6-(4-(5-((2-(2,6-dioxopiperidin-3-yl)-1,3-dioxoisoindolin-4-yl)oxy)pentanoyl)piperazin-1-yl)pyridazine-3-carbonyl)piperidin-4-yl)butyl)-3-(pyridin-3-yl)acrylamide;
(E)-N-(4-(1-(6-(4-(7-((2-(2,6-dioxopiperidin-3-yl)-1,3-dioxoisoindolin-4-yl)oxy)heptanoyl)piperazin-1-yl)pyridazine-3-carbonyl)piperidin-4-yl)butyl)-3-(pyridin-3-yl)acrylamide;
(E)-N-(4-(1-(6-(4-(5-(2-(2,6-dioxopiperidin-3-yl)-1,3-dioxoisoindolin-4-yl)pent-4-yn-1-yl)piperazin-1-yl)pyridazine-3-carbonyl)piperidin-4-yl)butyl)-3-(pyridin-3-yl)acrylamide;
(E)-N-(4-(1-(6-(4-(6-(2-(2,6-dioxopiperidin-3-yl)-1,3-dioxoisoindolin-4-yl)hex-5-yn-1-yl)piperazin-1-yl)pyridazine-3-carbonyl)piperidin-4-yl)butyl)-3-(pyridin-3-yl)acrylamide;
(E)-N-(4-(1-(6-(4-(7-(2-(2,6-dioxopiperidin-3-yl)-1,3-dioxoisoindolin-4-yl)hept-6-yn-1-yl)piperazin-1-yl)pyridazine-3-carbonyl)piperidin-4-yl)butyl)-3-(pyridin-3-yl)acrylamide;
(E)-N-(4-(1-(6-(4-(2-((2-(2,6-dioxopiperidin-3-yl)-1,3-dioxoisoindolin-4-yl)thio)ethyl)piperazin-1-yl)pyridazine-3-carbonyl)piperidin-4-yl)butyl)-3-(pyridin-3-yl)acrylamide;
(E)-N-(4-(1-(6-(4-(3-((2-(2,6-dioxopiperidin-3-yl)-1,3-dioxoisoindolin-4-yl)thio)propyl)piperazin-1-yl)pyridazine-3-carbonyl)piperidin-4-yl)butyl)-3-(pyridin-3-yl)acrylamide;
(E)-N-(4-(1-(6-(4-(5-((2-(2,6-dioxopiperidin-3-yl)-1,3-dioxoisoindolin-4-yl)thio)pentyl)piperazin-1-yl)pyridazine-3-carbonyl)piperidin-4-yl)butyl)-3-(pyridin-3-yl)acrylamide;
(E)-N-(4-(1-(6-(4-(6-((2-(2,6-dioxopiperidin-3-yl)-1,3-dioxoisoindolin-4-yl)thio)hexyl)piperazin-1-yl)pyridazine-3-carbonyl)piperidin-4-yl)butyl)-3-(pyridin-3-yl)acrylamide;
(E)-N-(4-(1-(6-(4-(4-((2-(2,6-dioxopiperidin-3-yl)-1,3-dioxoisoindolin-4-yl)oxy)butyl)piperazin-1-yl)pyridazine-3-carbonyl)piperidin-4-yl)butyl)-3-(pyridin-3-yl)acrylamide;
(E)-N-(4-(1-(6-(4-(8-((2-(2,6-dioxopiperidin-3-yl)-1,3-dioxoisoindolin-4-yl)oxy)octyl)piperazin-1-yl)pyridazine-3-carbonyl)piperidin-4-yl)butyl)-3-(pyridin-3-yl)acrylamide;
(E)-N-(4-(1-(6-(4-(6-(2-(2,6-dioxopiperidin-3-yl)-1,3-dioxoisoindolin-4-yl)hexyl)piperazin-1-yl)pyridazine-3-carbonyl)piperidin-4-yl)butyl)-3-(pyridin-3-yl)acrylamide;
(E)-N-(4-(1-(6-(4-(7-(2-(2,6-dioxopiperidin-3-yl)-1,3-dioxoisoindolin-4-yl)heptyl)piperazin-1-yl)pyridazine-3-carbonyl)piperidin-4-yl)butyl)-3-(pyridin-3-yl)acrylamide;
(E)-N-(4-(1-(6-(4-(5-(2-(2,6-dioxopiperidin-3-yl)-1,3-dioxoisoindolin-4-yl)pentyl)piperazin-1-yl)pyridazine-3-carbonyl)piperidin-4-yl)butyl)-3-(pyridin-3-yl)acrylamide;
(E)-N-(4-(1-(6-(4-((2-(2,6-dioxopiperidin-3-yl)-1-oxoisoindolin-4-yl)glycyl)piperazin-1-yl)pyridazine-3-carbonyl)piperidin-4-yl)butyl)-3-(pyridin-3-yl)acrylamide;
(E)-N-(4-(1-(6-(4-(4-((2-(2,6-dioxopiperidin-3-yl)-1-oxoisoindolin-4-yl)amino)butanoyl)piperazin-1-yl)pyridazine-3-carbonyl)piperidin-4-yl)butyl)-3-(pyridin-3-yl)acrylamide;
(E)-N-(4-(1-(6-(4-(6-((2-(2,6-dioxopiperidin-3-yl)-1-oxoisoindolin-4-yl)amino)hexanoyl)piperazin-1-yl)pyridazine-3-carbonyl)piperidin-4-yl)butyl)-3-(pyridin-3-yl)acrylamide;
(E)-N-(4-(1-(6-(4-(2-(2-((2-(2,6-dioxopiperidin-3-yl)-1-oxoisoindolin-4-yl)amino)ethoxy)acetyl)piperazin-1-yl)pyridazine-3-carbonyl)piperidin-4-yl)butyl)-3-(pyridin-3-yl)acrylamide;
(E)-N-(4-(1-(6-(4-(3-(2-((2-(2,6-dioxopiperidin-3-yl)-1-oxoisoindolin-4-yl)amino)ethoxy)propanoyl)piperazin-1-yl)pyridazine-3-carbonyl)piperidin-4-yl)butyl)-3-(pyridin-3-yl)acrylamide;
(E)-N-(4-(1-(6-(4-(2-(2-(2-((2-(2,6-dioxopiperidin-3-yl)-1-oxoisoindolin-4-yl)amino)ethoxy)ethoxy)acetyl)piperazin-1-yl)pyridazine-3-carbonyl)piperidin-4-yl)butyl)-3-(pyridin-3-yl)acrylamide
(E)-N-(4-(1-(6-(4-(3-(2-(2-((2-(2,6-dioxopiperidin-3-yl)-1-oxoisoindolin-4-yl)amino)ethoxy)ethoxy)propanoyl)piperazin-1-yl)pyridazine-3-carbonyl)piperidin-4-yl)butyl)-3-(pyridin-3-yl)acrylamide;
(E)-N-(4-(1-(6-(4-(14-((2-(2,6-dioxopiperidin-3-yl)-1-oxoisoindolin-4-yl)amino)-3,6,9,12-tetraoxatetradecanoyl)piperazin-1-yl)pyridazine-3-carbonyl)piperidin-4-yl)butyl)-3-(pyridin-3-yl)acrylamide;
(E)-N-(4-(1-(6-(4-(1-((2-(2,6-dioxopiperidin-3-yl)-1-oxoisoindolin-4-yl)amino)-3,6,9,12-tetraoxapentadecan-15-oyl)piperazin-1-yl)pyridazine-3-carbonyl)piperidin-4-yl)butyl)-3-(pyridin-3-yl)acrylamide;
(E)-N-(4-(1-(6-(4-(5-((2-(2,6-dioxopiperidin-3-yl)-1-oxoisoindolin-4-yl)thio)pentanoyl)piperazin-1-yl)pyridazine-3-carbonyl)piperidin-4-yl)butyl)-3-(pyridin-3-yl)acrylamide;
(E)-N-(4-(1-(6-(4-(6-((2-(2,6-dioxopiperidin-3-yl)-1-oxoisoindolin-4-yl)thio)hexanoyl)piperazin-1-yl)pyridazine-3-carbonyl)piperidin-4-yl)butyl)-3-(pyridin-3-yl)acrylamide;
(E)-N-(4-(1-(6-(4-(2-(2-((2-(2,6-dioxopiperidin-3-yl)-1-oxoisoindolin-4-yl)thio)ethoxy)acetyl)piperazin-1-yl)pyridazine-3-carbonyl)piperidin-4-yl)butyl)-3-(pyridin-3-yl)acrylamide;
(E)-N-(4-(1-(6-(4-(2-(2-(2-((2-(2,6-dioxopiperidin-3-yl)-1-oxoisoindolin-4-yl)thio)ethoxy)ethoxy)acetyl)piperazin-1-yl)pyridazine-3-carbonyl)piperidin-4-yl)butyl)-3-(pyridin-3-yl)acrylamide;
(E)-N-(4-(1-(6-(4-(17-((2-(2,6-dioxopiperidin-3-yl)-1-oxoisoindolin-4-yl)thio)-3,6,9,12,15-pentaoxaheptadecanoyl)piperazin-1-yl)pyridazine-3-carbonyl)piperidin-4-yl)butyl)-3-(pyridin-3-yl)acrylamide;
(E)-N-(4-(1-(6-(4-(2-((2-(2,6-dioxopiperidin-3-yl)-1-oxoisoindolin-4-yl)oxy)acetyl)piperazin-1-yl)pyridazine-3-carbonyl)piperidin-4-yl)butyl)-3-(pyridin-3-yl)acrylamide;
(E)-N-(4-(1-(6-(4-(5-((2-(2,6-dioxopiperidin-3-yl)-1-oxoisoindolin-4-yl)oxy)pentanoyl)piperazin-1-yl)pyridazine-3-carbonyl)piperidin-4-yl)butyl)-3-(pyridin-3-yl)acrylamide;
(E)-N-(4-(1-(6-(4-(7-((2-(2,6-dioxopiperidin-3-yl)-1-oxoisoindolin-4-yl)oxy)heptanoyl)piperazin-1-yl)pyridazine-3-carbonyl)piperidin-4-yl)butyl)-3-(pyridin-3-yl)acrylamide;
(E)-N-(4-(1-(6-(4-(5-(2-(2,6-dioxopiperidin-3-yl)-1-oxoisoindolin-4-yl)pent-4-yn-1-yl)piperazin-1-yl)pyridazine-3-carbonyl)piperidin-4-yl)butyl)-3-(pyridin-3-yl)acrylamide;
(E)-N-(4-(1-(6-(4-(6-(2-(2,6-dioxopiperidin-3-yl)-1-oxoisoindolin-4-yl)hex-5-yn-1-yl)piperazin-1-yl)pyridazine-3-carbonyl)piperidin-4-yl)butyl)-3-(pyridin-3-yl)acrylamide;
(E)-N-(4-(1-(6-(4-(9-(2-(2,6-dioxopiperidin-3-yl)-1-oxoisoindolin-4-yl)non-8-yn-1-yl)piperazin-1-yl)pyridazine-3-carbonyl)piperidin-4-yl)butyl)-3-(pyridin-3-yl)acrylamide;
(E)-N-(4-(1-(6-(4-(2-((2-(2,6-dioxopiperidin-3-yl)-1-oxoisoindolin-4-yl)thio)ethyl)piperazin-1-yl)pyridazine-3-carbonyl)piperidin-4-yl)butyl)-3-(pyridin-3-yl)acrylamide;
(E)-N-(4-(1-(6-(4-(3-((2-(2,6-dioxopiperidin-3-yl)-1-oxoisoindolin-4-yl)thio)propyl)piperazin-1-yl)pyridazine-3-carbonyl)piperidin-4-yl)butyl)-3-(pyridin-3-yl)acrylamide;
(E)-N-(4-(1-(6-(4-(5-((2-(2,6-dioxopiperidin-3-yl)-1-oxoisoindolin-4-yl)thio)pentyl)piperazin-1-yl)pyridazine-3-carbonyl)piperidin-4-yl)butyl)-3-(pyridin-3-yl)acrylamide;
(E)-N-(4-(1-(6-(4-(8-((2-(2,6-dioxopiperidin-3-yl)-1-oxoisoindolin-4-yl)thio)octyl)piperazin-1-yl)pyridazine-3-carbonyl)piperidin-4-yl)butyl)-3-(pyridin-3-yl)acrylamide;
(E)-N-(4-(1-(6-(4-(4-((2-(2,6-dioxopiperidin-3-yl)-1-oxoisoindolin-4-yl)oxy)butyl)piperazin-1-yl)pyridazine-3-carbonyl)piperidin-4-yl)butyl)-3-(pyridin-3-yl)acrylamide;
(E)-N-(4-(1-(6-(4-(8-((2-(2,6-dioxopiperidin-3-yl)-1-oxoisoindolin-4-yl)oxy)octyl)piperazin-1-yl)pyridazine-3-carbonyl)piperidin-4-yl)butyl)-3-(pyridin-3-yl)acrylamide;
(E)-N-(2,6-dioxopiperidin-3-yl)-3-(6-(4-(6-(4-(4-(3-(pyridin-3-yl)acrylamido)butyl)piperidine-1-carbonyl)pyridazin-3-yl)piperazin-1-yl)hexyl)benzamide;
(E)-N-(4-(1-(6-(4-(7-(2-(2,6-dioxopiperidin-3-yl)-1-oxoisoindolin-4-yl)heptyl)piperazin-1-yl)pyridazine-3-carbonyl)piperidin-4-yl)butyl)-3-(pyridin-3-yl)acrylamide;
(E)-N-(4-(1-(6-(4-(5-(2-(2,6-dioxopiperidin-3-yl)-1-oxoisoindolin-4-yl)pentyl)piperazin-1-yl)pyridazine-3-carbonyl)piperidin-4-yl)butyl)-3-(pyridin-3-yl)acrylamide;
(2S,4R)-1-((S)-3,3-dimethyl-2-(3-oxo-3-(4-(6-(4-(4-((E)-3-(pyridin-3-yl)acrylamido)butyl)piperidine-1-carbonyl)pyridazin-3-yl)piperazin-1-yl)propanamido)butanoyl)-4-hydroxy-N-(4-(4-methylthiazol-5-yl)benzyl)pyrrolidine-2-carboxamide;
(2S,4R)-1-((S)-3,3-dimethyl-2-(5-oxo-5-(4-(6-(4-(4-((E)-3-(pyridin-3-yl)acrylamido)butyl)piperidine-1-carbonyl)pyridazin-3-yl)piperazin-1-yl)pentanamido)butanoyl)-4-hydroxy-N-(4-(4-methylthiazol-5-yl)benzyl)pyrrolidine-2-carboxamide;
(2S,4R)-1-((S)-3,3-dimethyl-2-(7-oxo-7-(4-(6-(4-(4-((E)-3-(pyridin-3-yl)acrylamido)butyl)piperidine-1-carbonyl)pyridazin-3-yl)piperazin-1-yl)heptanamido)butanoyl)-4-hydroxy-N-(4-(4-methylthiazol-5-yl)benzyl)pyrrolidine-2-carboxamide;
(2S,4R)-1-((S)-3,3-dimethyl-2-(2-(2-(2-oxo-2-(4-(6-(4-(4-((E)-3-(pyridin-3-yl)acrylamido)butyl)piperidine-1-carbonyl)pyridazin-3-yl)piperazin-1-yl)ethoxy)ethoxy)acetamido)butanoyl)-4-hydroxy-N-(4-(4-methylthiazol-5-yl)benzyl)pyrrolidine-2-carboxamide;
(2S,4R)-1-((S)-3,3-dimethyl-2-(3-(2-(3-oxo-3-(4-(6-(4-(4-((E)-3-(pyridin-3-yl)acrylamido)butyl)piperidine-1-carbonyl)pyridazin-3-yl)piperazin-1-yl)propoxy)ethoxy)propanamido)butanoyl)-4-hydroxy-N-(4-(4-methylthiazol-5-yl)benzyl)pyrrolidine-2-carboxamide;
(2S,4R)-1-((S)-2-(tert-butyl)-4,19-dioxo-19-(4-(6-(4-(4-((E)-3-(pyridin-3-yl)acrylamido)butyl)piperidine-1-carbonyl)pyridazin-3-yl)piperazin-1-yl)-7,10,13,16-tetraoxa-3-azanonadecanoyl)-4-hydroxy-N-(4-(4-methylthiazol-5-yl)benzyl)pyrrolidine-2-carboxamide;
(2S,4R)-1-((S)-2-(tert-butyl)-4,22-dioxo-22-(4-(6-(4-(4-((E)-3-(pyridin-3-yl)acrylamido)butyl)piperidine-1-carbonyl)pyridazin-3-yl)piperazin-1-yl)-7,10,13,16,19-pentaoxa-3-azadocosanoyl)-4-hydroxy-N-(4-(4-methylthiazol-5-yl)benzyl)pyrrolidine-2-carboxamide;
(E)-N-(4-(1-(6-(4-(4-(3-((2-(2,6-dioxopiperidin-3-yl)-1,3-dioxoisoindolin-4-yl)amino)propanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(5-((2-(2,6-dioxopiperidin-3-yl)-1,3-dioxoisoindolin-4-yl)amino)pentanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(7-((2-(2,6-dioxopiperidin-3-yl)-1,3-dioxoisoindolin-4-yl)amino)heptanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(3-(2-((2-(2,6-dioxopiperidin-3-yl)-1,3-dioxoisoindolin-4-yl)amino)ethoxy)propanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(3-(2-(2-((2-(2,6-dioxopiperidin-3-yl)-1,3-dioxoisoindolin-4-yl)amino)ethoxy)ethoxy)propanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(2-((2-(2,6-dioxopiperidin-3-yl)-1,3-dioxoisoindolin-4-yl)thio)acetyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(3-((2-(2,6-dioxopiperidin-3-yl)-1,3-dioxoisoindolin-4-yl)thio)propanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(6-((2-(2,6-dioxopiperidin-3-yl)-1,3-dioxoisoindolin-4-yl)thio)hexanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(7-((2-(2,6-dioxopiperidin-3-yl)-1,3-dioxoisoindolin-4-yl)thio)heptanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(2-(2-((2-(2,6-dioxopiperidin-3-yl)-1,3-dioxoisoindolin-4-yl)thio)ethoxy)acetyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(3-(2-((2-(2,6-dioxopiperidin-3-yl)-1,3-dioxoisoindolin-4-yl)thio)ethoxy)propanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(2-(2-(2-(2-((2-(2,6-dioxopiperidin-3-yl)-1,3-dioxoisoindolin-4-yl)thio)ethoxy)ethoxy)ethoxy)acetyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(3-(2-(2-(2-((2-(2,6-dioxopiperidin-3-yl)-1,3-dioxoisoindolin-4-yl)thio)ethoxy)ethoxy)ethoxy)propanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(14-((2-(2,6-dioxopiperidin-3-yl)-1,3-dioxoisoindolin-4-yl)thio)-3,6,9,12-tetraoxatetradecanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(17-((2-(2,6-dioxopiperidin-3-yl)-1,3-dioxoisoindolin-4-yl)thio)-3,6,9,12,15-pentaoxaheptadecanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(2-((2-(2,6-dioxopiperidin-3-yl)-1,3-dioxoisoindolin-4-yl)oxy)acetyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(5-((2-(2,6-dioxopiperidin-3-yl)-1,3-dioxoisoindolin-4-yl)oxy)pentanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(7-((2-(2,6-dioxopiperidin-3-yl)-1,3-dioxoisoindolin-4-yl)oxy)heptanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(5-(2-(2,6-dioxopiperidin-3-yl)-1,3-dioxoisoindolin-4-yl)pent-4-yn-1-yl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(6-(2-(2,6-dioxopiperidin-3-yl)-1,3-dioxoisoindolin-4-yl)hex-5-yn-1-yl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(7-(2-(2,6-dioxopiperidin-3-yl)-1,3-dioxoisoindolin-4-yl)hept-6-yn-1-yl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(2-((2-(2,6-dioxopiperidin-3-yl)-1,3-dioxoisoindolin-4-yl)thio)ethyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(3-((2-(2,6-dioxopiperidin-3-yl)-1,3-dioxoisoindolin-4-yl)thio)propyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(5-((2-(2,6-dioxopiperidin-3-yl)-1,3-dioxoisoindolin-4-yl)thio)pentyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(6-((2-(2,6-dioxopiperidin-3-yl)-1,3-dioxoisoindolin-4-yl)thio)hexyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(4-((2-(2,6-dioxopiperidin-3-yl)-1,3-dioxoisoindolin-4-yl)oxy)butyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(8-((2-(2,6-dioxopiperidin-3-yl)-1,3-dioxoisoindolin-4-yl)oxy)octyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(6-(2-(2,6-dioxopiperidin-3-yl)-1,3-dioxoisoindolin-4-yl)hexyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(7-(2-(2,6-dioxopiperidin-3-yl)-1,3-dioxoisoindolin-4-yl)heptyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(5-(2-(2,6-dioxopiperidin-3-yl)-1,3-dioxoisoindolin-4-yl)pentyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-((2-(2,6-dioxopiperidin-3-yl)-1-oxoisoindolin-4-yl)glycyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(4-((2-(2,6-dioxopiperidin-3-yl)-1-oxoisoindolin-4-yl)amino)butanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(6-((2-(2,6-dioxopiperidin-3-yl)-1-oxoisoindolin-4-yl)amino)hexanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(2-(2-((2-(2,6-dioxopiperidin-3-yl)-1-oxoisoindolin-4-yl)amino)ethoxy)acetyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(3-(2-((2-(2,6-dioxopiperidin-3-yl)-1-oxoisoindolin-4-yl)amino)ethoxy)propanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(2-(2-(2-((2-(2,6-dioxopiperidin-3-yl)-1-oxoisoindolin-4-yl)amino)ethoxy)ethoxy)acetyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(3-(2-(2-((2-(2,6-dioxopiperidin-3-yl)-1-oxoisoindolin-4-yl)amino)ethoxy)ethoxy)propanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(14-((2-(2,6-dioxopiperidin-3-yl)-1-oxoisoindolin-4-yl)amino)-3,6,9,12-tetraoxatetradecanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(1-((2-(2,6-dioxopiperidin-3-yl)-1-oxoisoindolin-4-yl)amino)-3,6,9,12-tetraoxapentadecan-15-oyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(5-((2-(2,6-dioxopiperidin-3-yl)-1-oxoisoindolin-4-yl)thio)pentanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(6-((2-(2,6-dioxopiperidin-3-yl)-1-oxoisoindolin-4-yl)thio)hexanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(2-(2-((2-(2,6-dioxopiperidin-3-yl)-1-oxoisoindolin-4-yl)thio)ethoxy)acetyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(2-(2-(2-((2-(2,6-dioxopiperidin-3-yl)-1-oxoisoindolin-4-yl)thio)ethoxy)ethoxy)acetyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(17-((2-(2,6-dioxopiperidin-3-yl)-1-oxoisoindolin-4-yl)thio)-3,6,9,12,15-pentaoxaheptadecanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(2-((2-(2,6-dioxopiperidin-3-yl)-1-oxoisoindolin-4-yl)oxy)acetyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(5-((2-(2,6-dioxopiperidin-3-yl)-1-oxoisoindolin-4-yl)oxy)pentanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(7-((2-(2,6-dioxopiperidin-3-yl)-1-oxoisoindolin-4-yl)oxy)heptanoyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(5-(2-(2,6-dioxopiperidin-3-yl)-1-oxoisoindolin-4-yl)pent-4-yn-1-yl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(6-(2-(2,6-dioxopiperidin-3-yl)-1-oxoisoindolin-4-yl)hex-5-yn-1-yl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(9-(2-(2,6-dioxopiperidin-3-yl)-1-oxoisoindolin-4-yl)non-8-yn-1-yl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(2-((2-(2,6-dioxopiperidin-3-yl)-1-oxoisoindolin-4-yl)thio)ethyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(3-((2-(2,6-dioxopiperidin-3-yl)-1-oxoisoindolin-4-yl)thio)propyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(5-((2-(2,6-dioxopiperidin-3-yl)-1-oxoisoindolin-4-yl)thio)pentyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(8-((2-(2,6-dioxopiperidin-3-yl)-1-oxoisoindolin-4-yl)thio)octyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(4-((2-(2,6-dioxopiperidin-3-yl)-1-oxoisoindolin-4-yl)oxy)butyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(8-((2-(2,6-dioxopiperidin-3-yl)-1-oxoisoindolin-4-yl)oxy)octyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(6-(2-(2,6-dioxopiperidin-3-yl)-1-oxoisoindolin-4-yl)hexyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(7-(2-(2,6-dioxopiperidin-3-yl)-1-oxoisoindolin-4-yl)heptyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(5-(2-(2,6-dioxopiperidin-3-yl)-1-oxoisoindolin-4-yl)pentyl)piperazin-1-yl)piperidin-1-yl)pyridazine-3-carbonyl)piperidin-4-yl)butyl)-3-(pyridin-3-yl)acrylamide;
(2S,4R)-1-((S)-3,3-dimethyl-2-(3-oxo-3-(4-(1-(6-(4-(4-((E)-3-(pyridin-3-yl)acrylamido)butyl)piperidine-1-carbonyl)pyridazin-3-yl)piperidin-4-yl)piperazin-1-yl)propanamido)butanoyl)-4-hydroxy-N-(4-(4-methylthiazol-5-yl)benzyl)pyrrolidine-2-carboxamide;
(2S,4R)-1-((S)-3,3-dimethyl-2-(5-oxo-5-(4-(1-(6-(4-(4-((E)-3-(pyridin-3-yl)acrylamido)butyl)piperidine-1-carbonyl)pyridazin-3-yl)piperidin-4-yl)piperazin-1-yl)pentanamido)butanoyl)-4-hydroxy-N-(4-(4-methylthiazol-5-yl)benzyl)pyrrolidine-2-carboxamide;
(2S,4R)-1-((S)-3,3-dimethyl-2-(7-oxo-7-(4-(1-(6-(4-(4-((E)-3-(pyridin-3-yl)acrylamido)butyl)piperidine-1-carbonyl)pyridazin-3-yl)piperidin-4-yl)piperazin-1-yl)heptanamido)butanoyl)-4-hydroxy-N-(4-(4-methylthiazol-5-yl)benzyl)pyrrolidine-2-carboxamide;
(2S,4R)-1-((S)-3,3-dimethyl-2-(8-oxo-8-(4-(1-(6-(4-(4-((E)-3-(pyridin-3-yl)acrylamido)butyl)piperidine-1-carbonyl)pyridazin-3-yl)piperidin-4-yl)piperazin-1-yl)octanamido)butanoyl)-4-hydroxy-N-(4-(4-methylthiazol-5-yl)benzyl)pyrrolidine-2-carboxamide;
(2S,4R)-1-((S)-3,3-dimethyl-2-(11-oxo-11-(4-(1-(6-(4-(4-((E)-3-(pyridin-3-yl)acrylamido)butyl)piperidine-1-carbonyl)pyridazin-3-yl)piperidin-4-yl)piperazin-1-yl)undecanamido)butanoyl)-4-hydroxy-N-(4-(4-methylthiazol-5-yl)benzyl)pyrrolidine-2-carboxamide;
(2S,4R)-1-((S)-3,3-dimethyl-2-(2-(2-(2-oxo-2-(4-(1-(6-(4-(4-((E)-3-(pyridin-3-yl)acrylamido)butyl)piperidine-1-carbonyl)pyridazin-3-yl)piperidin-4-yl)piperazin-1-yl)ethoxy)ethoxy)acetamido)butanoyl)-4-hydroxy-N-(4-(4-methylthiazol-5-yl)benzyl)pyrrolidine-2-carboxamide;
(2S,4R)-1-((S)-3,3-dimethyl-2-(3-(2-(3-oxo-3-(4-(1-(6-(4-(4-((E)-3-(pyridin-3-yl)acrylamido)butyl)piperidine-1-carbonyl)pyridazin-3-yl)piperidin-4-yl)piperazin-1-yl)propoxy)ethoxy)propanamido)butanoyl)-4-hydroxy-N-(4-(4-methylthiazol-5-yl)benzyl)pyrrolidine-2-carboxamide;
(2S,4R)-1-((S)-2-(tert-butyl)-4,19-dioxo-19-(4-(1-(6-(4-(4-((E)-3-(pyridin-3-yl)acrylamido)butyl)piperidine-1-carbonyl)pyridazin-3-yl)piperidin-4-yl)piperazin-1-yl)-7,10,13,16-tetraoxa-3-azanonadecanoyl)-4-hydroxy-N-(4-(4-methylthiazol-5-yl)benzyl)pyrrolidine-2-carboxamide;
(E)-N-(4-(1-(6-(4-(4-(3-((2-(2,6-dioxopiperidin-3-yl)-1,3-dioxoisoindolin-4-yl)amino)propanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(5-((2-(2,6-dioxopiperidin-3-yl)-1,3-dioxoisoindolin-4-yl)amino)pentanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(7-((2-(2,6-dioxopiperidin-3-yl)-1,3-dioxoisoindolin-4-yl)amino)heptanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(3-(2-((2-(2,6-dioxopiperidin-3-yl)-1,3-dioxoisoindolin-4-yl)amino)ethoxy)propanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(3-(2-(2-((2-(2,6-dioxopiperidin-3-yl)-1,3-dioxoisoindolin-4-yl)amino)ethoxy)ethoxy)propanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(2-((2-(2,6-dioxopiperidin-3-yl)-1,3-dioxoisoindolin-4-yl)thio)acetyl)piperazin-1-yl)piperidin-1-yl)nicotinoyl)piperidin-4-yl)butyl)-3-(pyridin-3-yl)acrylamide;
(E)-N-(4-(1-(6-(4-(4-(3-((2-(2,6-dioxopiperidin-3-yl)-1,3-dioxoisoindolin-4-yl)thio)propanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(6-((2-(2,6-dioxopiperidin-3-yl)-1,3-dioxoisoindolin-4-yl)thio)hexanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(7-((2-(2,6-dioxopiperidin-3-yl)-1,3-dioxoisoindolin-4-yl)thio)heptanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(2-(2-((2-(2,6-dioxopiperidin-3-yl)-1,3-dioxoisoindolin-4-yl)thio)ethoxy)acetyl)piperazin-1-yl)piperidin-1-yl)nicotinoyl)piperidin-4-yl)butyl)-3-(pyridin-3-yl)acrylamide;
(E)-N-(4-(1-(6-(4-(4-(3-(2-((2-(2,6-dioxopiperidin-3-yl)-1,3-dioxoisoindolin-4-yl)thio)ethoxy)propanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(2-(2-(2-(2-((2-(2,6-dioxopiperidin-3-yl)-1,3-dioxoisoindolin-4-yl)thio)ethoxy)ethoxy)ethoxy)acetyl)piperazin-1-yl)piperidin-1-yl)nicotinoyl)piperidin-4-yl)butyl)-3-(pyridin-3-yl)acrylamide;
(E)-N-(4-(1-(6-(4-(4-(3-(2-(2-(2-((2-(2,6-dioxopiperidin-3-yl)-1,3-dioxoisoindolin-4-yl)thio)ethoxy)ethoxy)ethoxy)propanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(14-((2-(2,6-dioxopiperidin-3-yl)-1,3-dioxoisoindolin-4-yl)thio)-3,6,9,12-tetraoxatetradecanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(17-((2-(2,6-dioxopiperidin-3-yl)-1,3-dioxoisoindolin-4-yl)thio)-3,6,9,12,15-pentaoxaheptadecanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(2-((2-(2,6-dioxopiperidin-3-yl)-1,3-dioxoisoindolin-4-yl)oxy)acetyl)piperazin-1-yl)piperidin-1-yl)nicotinoyl)piperidin-4-yl)butyl)-3-(pyridin-3-yl)acrylamide;
(E)-N-(4-(1-(6-(4-(4-(5-((2-(2,6-dioxopiperidin-3-yl)-1,3-dioxoisoindolin-4-yl)oxy)pentanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(7-((2-(2,6-dioxopiperidin-3-yl)-1,3-dioxoisoindolin-4-yl)oxy)heptanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(5-(2-(2,6-dioxopiperidin-3-yl)-1,3-dioxoisoindolin-4-yl)pent-4-yn-1-yl)piperazin-1-yl)piperidin-1-yl)nicotinoyl)piperidin-4-yl)butyl)-3-(pyridin-3-yl)acrylamide;
(E)-N-(4-(1-(6-(4-(4-(6-(2-(2,6-dioxopiperidin-3-yl)-1,3-dioxoisoindolin-4-yl)hex-5-yn-1-yl)piperazin-1-yl)piperidin-1-yl)nicotinoyl)piperidin-4-yl)butyl)-3-(pyridin-3-yl)acrylamide;
(E)-N-(4-(1-(6-(4-(4-(7-(2-(2,6-dioxopiperidin-3-yl)-1,3-dioxoisoindolin-4-yl)hept-6-yn-1-yl)piperazin-1-yl)piperidin-1-yl)nicotinoyl)piperidin-4-yl)butyl)-3-(pyridin-3-yl)acrylamide;
(E)-N-(4-(1-(6-(4-(4-(2-((2-(2,6-dioxopiperidin-3-yl)-1,3-dioxoisoindolin-4-yl)thio)ethyl)piperazin-1-yl)piperidin-1-yl)nicotinoyl)piperidin-4-yl)butyl)-3-(pyridin-3-yl)acrylamide;
(E)-N-(4-(1-(6-(4-(4-(3-((2-(2,6-dioxopiperidin-3-yl)-1,3-dioxoisoindolin-4-yl)thio)propyl)piperazin-1-yl)piperidin-1-yl)nicotinoyl)piperidin-4-yl)butyl)-3-(pyridin-3-yl)acrylamide;
(E)-N-(4-(1-(6-(4-(4-(5-((2-(2,6-dioxopiperidin-3-yl)-1,3-dioxoisoindolin-4-yl)thio)pentyl)piperazin-1-yl)piperidin-1-yl)nicotinoyl)piperidin-4-yl)butyl)-3-(pyridin-3-yl)acrylamide;
(E)-N-(4-(1-(6-(4-(4-(6-((2-(2,6-dioxopiperidin-3-yl)-1,3-dioxoisoindolin-4-yl)thio)hexyl)piperazin-1-yl)piperidin-1-yl)nicotinoyl)piperidin-4-yl)butyl)-3-(pyridin-3-yl)acrylamide;
(E)-N-(4-(1-(6-(4-(4-(4-((2-(2,6-dioxopiperidin-3-yl)-1,3-dioxoisoindolin-4-yl)oxy)butyl)piperazin-1-yl)piperidin-1-yl)nicotinoyl)piperidin-4-yl)butyl)-3-(pyridin-3-yl)acrylamide;
(E)-N-(4-(1-(6-(4-(4-(8-((2-(2,6-dioxopiperidin-3-yl)-1,3-dioxoisoindolin-4-yl)oxy)octyl)piperazin-1-yl)piperidin-1-yl)nicotinoyl)piperidin-4-yl)butyl)-3-(pyridin-3-yl)acrylamide;
(E)-N-(4-(1-(6-(4-(4-(6-(2-(2,6-dioxopiperidin-3-yl)-1,3-dioxoisoindolin-4-yl)hexyl)piperazin-1-yl)piperidin-1-yl)nicotinoyl)piperidin-4-yl)butyl)-3-(pyridin-3-yl)acrylamide;
(E)-N-(4-(1-(6-(4-(4-(7-(2-(2,6-dioxopiperidin-3-yl)-1,3-dioxoisoindolin-4-yl)heptyl)piperazin-1-yl)piperidin-1-yl)nicotinoyl)piperidin-4-yl)butyl)-3-(pyridin-3-yl)acrylamide;
(E)-N-(4-(1-(6-(4-(4-(5-(2-(2,6-dioxopiperidin-3-yl)-1,3-dioxoisoindolin-4-yl)pentyl)piperazin-1-yl)piperidin-1-yl)nicotinoyl)piperidin-4-yl)butyl)-3-(pyridin-3-yl)acrylamide;
(E)-N-(4-(1-(6-(4-(4-((2-(2,6-dioxopiperidin-3-yl)-1-oxoisoindolin-4-yl)glycyl)piperazin-1-yl)piperidin-1-yl)nicotinoyl)piperidin-4-yl)butyl)-3-(pyridin-3-yl)acrylamide;
(E)-N-(4-(1-(6-(4-(4-(4-((2-(2,6-dioxopiperidin-3-yl)-1-oxoisoindolin-4-yl)amino)butanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(6-((2-(2,6-dioxopiperidin-3-yl)-1-oxoisoindolin-4-yl)amino)hexanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(2-(2-((2-(2,6-dioxopiperidin-3-yl)-1-oxoisoindolin-4-yl)amino)ethoxy)acetyl)piperazin-1-yl)piperidin-1-yl)nicotinoyl)piperidin-4-yl)butyl)-3-(pyridin-3-yl)acrylamide;
(E)-N-(4-(1-(6-(4-(4-(3-(2-((2-(2,6-dioxopiperidin-3-yl)-1-oxoisoindolin-4-yl)amino)ethoxy)propanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(2-(2-(2-((2-(2,6-dioxopiperidin-3-yl)-1-oxoisoindolin-4-yl)amino)ethoxy)ethoxy)acetyl)piperazin-1-yl)piperidin-1-yl)nicotinoyl)piperidin-4-yl)butyl)-3-(pyridin-3-yl)acrylamide;
(E)-N-(4-(1-(6-(4-(4-(3-(2-(2-((2-(2,6-dioxopiperidin-3-yl)-1-oxoisoindolin-4-yl)amino)ethoxy)ethoxy)propanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(14-((2-(2,6-dioxopiperidin-3-yl)-1-oxoisoindolin-4-yl)amino)-3,6,9,12-tetraoxatetradecanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(1-((2-(2,6-dioxopiperidin-3-yl)-1-oxoisoindolin-4-yl)amino)-3,6,9,12-tetraoxapentadecan-15-oyl)piperazin-1-yl)piperidin-1-yl)nicotinoyl)piperidin-4-yl)butyl)-3-(pyridin-3-yl)acrylamide;
(E)-N-(4-(1-(6-(4-(4-(5-((2-(2,6-dioxopiperidin-3-yl)-1-oxoisoindolin-4-yl)thio)pentanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(6-((2-(2,6-dioxopiperidin-3-yl)-1-oxoisoindolin-4-yl)thio)hexanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(2-(2-((2-(2,6-dioxopiperidin-3-yl)-1-oxoisoindolin-4-yl)thio)ethoxy)acetyl)piperazin-1-yl)piperidin-1-yl)nicotinoyl)piperidin-4-yl)butyl)-3-(pyridin-3-yl)acrylamide;
(E)-N-(4-(1-(6-(4-(4-(2-(2-(2-((2-(2,6-dioxopiperidin-3-yl)-1-oxoisoindolin-4-yl)thio)ethoxy)ethoxy)acetyl)piperazin-1-yl)piperidin-1-yl)nicotinoyl)piperidin-4-yl)butyl)-3-(pyridin-3-yl)acrylamide;
(E)-N-(4-(1-(6-(4-(4-(17-((2-(2,6-dioxopiperidin-3-yl)-1-oxoisoindolin-4-yl)thio)-3,6,9,12,15-pentaoxaheptadecanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(2-((2-(2,6-dioxopiperidin-3-yl)-1-oxoisoindolin-4-yl)oxy)acetyl)piperazin-1-yl)piperidin-1-yl)nicotinoyl)piperidin-4-yl)butyl)-3-(pyridin-3-yl)acrylamide;
(E)-N-(4-(1-(6-(4-(4-(5-((2-(2,6-dioxopiperidin-3-yl)-1-oxoisoindolin-4-yl)oxy)pentanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(7-((2-(2,6-dioxopiperidin-3-yl)-1-oxoisoindolin-4-yl)oxy)heptanoyl)piperazin-1-yl)piperidin-1-yl)nicotinoyl)piperidin-4-yl)butyl)-3-(pyridin-3-yl)acrylamide;
(E)-N-(4-(1-(6-(4-(4-(5-(2-(2,6-dioxopiperidin-3-yl)-1-oxoisoindolin-4-yl)pent-4-yn-1-yl)piperazin-1-yl)piperidin-1-yl)nicotinoyl)piperidin-4-yl)butyl)-3-(pyridin-3-yl)acrylamide;
(E)-N-(4-(1-(6-(4-(4-(6-(2-(2,6-dioxopiperidin-3-yl)-1-oxoisoindolin-4-yl)hex-5-yn-1-yl)piperazin-1-yl)piperidin-1-yl)nicotinoyl)piperidin-4-yl)butyl)-3-(pyridin-3-yl)acrylamide;
(E)-N-(4-(1-(6-(4-(4-(9-(2-(2,6-dioxopiperidin-3-yl)-1-oxoisoindolin-4-yl)non-8-yn-1-yl)piperazin-1-yl)piperidin-1-yl)nicotinoyl)piperidin-4-yl)butyl)-3-(pyridin-3-yl)acrylamide;
(E)-N-(4-(1-(6-(4-(4-(2-((2-(2,6-dioxopiperidin-3-yl)-1-oxoisoindolin-4-yl)thio)ethyl)piperazin-1-yl)piperidin-1-yl)nicotinoyl)piperidin-4-yl)butyl)-3-(pyridin-3-yl)acrylamide;
(E)-N-(4-(1-(6-(4-(4-(3-((2-(2,6-dioxopiperidin-3-yl)-1-oxoisoindolin-4-yl)thio)propyl)piperazin-1-yl)piperidin-1-yl)nicotinoyl)piperidin-4-yl)butyl)-3-(pyridin-3-yl)acrylamide;
(E)-N-(4-(1-(6-(4-(4-(5-((2-(2,6-dioxopiperidin-3-yl)-1-oxoisoindolin-4-yl)thio)pentyl)piperazin-1-yl)piperidin-1-yl)nicotinoyl)piperidin-4-yl)butyl)-3-(pyridin-3-yl)acrylamide;
(E)-N-(4-(1-(6-(4-(4-(8-((2-(2,6-dioxopiperidin-3-yl)-1-oxoisoindolin-4-yl)thio)octyl)piperazin-1-yl)piperidin-1-yl)nicotinoyl)piperidin-4-yl)butyl)-3-(pyridin-3-yl)acrylamide;
(E)-N-(4-(1-(6-(4-(4-(4-((2-(2,6-dioxopiperidin-3-yl)-1-oxoisoindolin-4-yl)oxy)butyl)piperazin-1-yl)piperidin-1-yl)nicotinoyl)piperidin-4-yl)butyl)-3-(pyridin-3-yl)acrylamide;
(E)-N-(4-(1-(6-(4-(4-(8-((2-(2,6-dioxopiperidin-3-yl)-1-oxoisoindolin-4-yl)oxy)octyl)piperazin-1-yl)piperidin-1-yl)nicotinoyl)piperidin-4-yl)butyl)-3-(pyridin-3-yl)acrylamide;
(E)-N-(4-(1-(6-(4-(4-(6-(2-(2,6-dioxopiperidin-3-yl)-1-oxoisoindolin-4-yl)hexyl)piperazin-1-yl)piperidin-1-yl)nicotinoyl)piperidin-4-yl)butyl)-3-(pyridin-3-yl)acrylamide;
(E)-N-(4-(1-(6-(4-(4-(7-(2-(2,6-dioxopiperidin-3-yl)-1-oxoisoindolin-4-yl)heptyl)piperazin-1-yl)piperidin-1-yl)nicotinoyl)piperidin-4-yl)butyl)-3-(pyridin-3-yl)acrylamide;
(E)-N-(4-(1-(6-(4-(4-(5-(2-(2,6-dioxopiperidin-3-yl)-1-oxoisoindolin-4-yl)pentyl)piperazin-1-yl)piperidin-1-yl)nicotinoyl)piperidin-4-yl)butyl)-3-(pyridin-3-yl)acrylamide;
(2S,4R)-1-((S)-3,3-dimethyl-2-(3-oxo-3-(4-(1-(5-(4-(4-((E)-3-(pyridin-3-yl)acrylamido)butyl)piperidine-1-carbonyl)pyridin-2-yl)piperidin-4-yl)piperazin-1-yl)propanamido)butanoyl)-4-hydroxy-N-(4-(4-methylthiazol-5-yl)benzyl)pyrrolidine-2-carboxamide;
(2S,4R)-1-((S)-3,3-dimethyl-2-(5-oxo-5-(4-(1-(5-(4-(4-((E)-3-(pyridin-3-yl)acrylamido)butyl)piperidine-1-carbonyl)pyridin-2-yl)piperidin-4-yl)piperazin-1-yl)pentanamido)butanoyl)-4-hydroxy-N-(4-(4-methylthiazol-5-yl)benzyl)pyrrolidine-2-carboxamide;
(2S,4R)-1-((S)-3,3-dimethyl-2-(7-oxo-7-(4-(1-(5-(4-(4-((E)-3-(pyridin-3-yl)acrylamido)butyl)piperidine-1-carbonyl)pyridin-2-yl)piperidin-4-yl)piperazin-1-yl)heptanamido)butanoyl)-4-hydroxy-N-(4-(4-methylthiazol-5-yl)benzyl)pyrrolidine-2-carboxamide;
(2S,4R)-1-((S)-3,3-dimethyl-2-(8-oxo-8-(4-(1-(5-(4-(4-((E)-3-(pyridin-3-yl)acrylamido)butyl)piperidine-1-carbonyl)pyridin-2-yl)piperidin-4-yl)piperazin-1-yl)octanamido)butanoyl)-4-hydroxy-N-(4-(4-methylthiazol-5-yl)benzyl)pyrrolidine-2-carboxamide;
(2S,4R)-1-((S)-3,3-dimethyl-2-(11-oxo-11-(4-(1-(5-(4-(4-((E)-3-(pyridin-3-yl)acrylamido)butyl)piperidine-1-carbonyl)pyridin-2-yl)piperidin-4-yl)piperazin-1-yl)undecanamido)butanoyl)-4-hydroxy-N-(4-(4-methylthiazol-5-yl)benzyl)pyrrolidine-2-carboxamide;
(2S,4R)-1-((S)-3,3-dimethyl-2-(2-(2-(2-oxo-2-(4-(1-(5-(4-(4-((E)-3-(pyridin-3-yl)acrylamido)butyl)piperidine-1-carbonyl)pyridin-2-yl)piperidin-4-yl)piperazin-1-yl)ethoxy)ethoxy)acetamido)butanoyl)-4-hydroxy-N-(4-(4-methylthiazol-5-yl)benzyl)pyrrolidine-2-carboxamide;
(2S,4R)-1-((S)-3,3-dimethyl-2-(2-(2-(2-oxo-2-(4-(1-(5-(4-(4-((E)-3-(pyridin-3-yl)acrylamido)butyl)piperidine-1-carbonyl)pyridin-2-yl)piperidin-4-yl)piperazin-1-yl)ethoxy)ethoxy)acetamido)butanoyl)-4-hydroxy-N-(4-(4-methylthiazol-5-yl)benzyl)pyrrolidine-2-carboxamide;
(2S,4R)-1-((S)-2-(tert-butyl)-4,19-dioxo-19-(4-(1-(5-(4-(4-((E)-3-(pyridin-3-yl)acrylamido)butyl)piperidine-1-carbonyl)pyridin-2-yl)piperidin-4-yl)piperazin-1-yl)-7,10,13,16-tetraoxa-3-azanonadecanoyl)-4-hydroxy-N-(4-(4-methylthiazol-5-yl)benzyl)pyrrolidine-2-carboxamide;
(E)-N-(4-(1-(6-(4-(4-(4-(2-(2,6-dioxopiperidin-3-yl)-1,3-dioxoisoindolin-5-yl)but-3-yn-1-yl)piperazin-1-yl)piperidin-1-yl)nicotinoyl)piperidin-4-yl)butyl)-3-(pyridin-3-yl)acrylamide;
(E)-N-(4-(1-(6-(4-(4-(7-(2-(2,6-dioxopiperidin-3-yl)-1,3-dioxoisoindolin-5-yl)hept-6-yn-1-yl)piperazin-1-yl)piperidin-1-yl)nicotinoyl)piperidin-4-yl)butyl)-3-(pyridin-3-yl)acrylamide;
(E)-N-(4-(1-(6-(4-(4-(7-((2-(2,6-dioxopiperidin-3-yl)-1,3-dioxoisoindolin-5-yl)thio)heptyl)piperazin-1-yl)piperidin-1-yl)nicotinoyl)piperidin-4-yl)butyl)-3-(pyridin-3-yl)acrylamide;
(E)-N-(4-(1-(6-(4-(4-(6-((2-(2,6-dioxopiperidin-3-yl)-1,3-dioxoisoindolin-5-yl)thio)hexyl)piperazin-1-yl)piperidin-1-yl)nicotinoyl)piperidin-4-yl)butyl)-3-(pyridin-3-yl)acrylamide;
(E)-N-(4-(1-(6-(4-(4-(5-((2-(2,6-dioxopiperidin-3-yl)-1,3-dioxoisoindolin-5-yl)thio)pentyl)piperazin-1-yl)piperidin-1-yl)nicotinoyl)piperidin-4-yl)butyl)-3-(pyridin-3-yl)acrylamide;
(E)-N-(4-(1-(6-(4-(4-(4-((2-(2,6-dioxopiperidin-3-yl)-1,3-dioxoisoindolin-5-yl)ethynyl)benzyl)piperazin-1-yl)piperidin-1-yl)nicotinoyl)piperidin-4-yl)butyl)-3-(pyridin-3-yl)acrylamide;
(E)-N-(4-(1-(6-(4-(4-(5-(2-(2,6-dioxopiperidin-3-yl)-3-oxoisoindolin-4-yl)pent-4-yn-1-yl)piperazin-1-yl)piperidin-1-yl)nicotinoyl)piperidin-4-yl)butyl)-3-(pyridin-3-yl)acrylamide;
(E)-N-(4-(1-(6-(4-(4-(4-((2-(2,6-dioxopiperidin-3-yl)-3-oxoisoindolin-4-yl)thio)butyl)piperazin-1-yl)piperidin-1-yl)nicotinoyl)piperidin-4-yl)butyl)-3-(pyridin-3-yl)acrylamide;
(E)-N-(4-(1-(6-(4-(4-(4-((2-(2,6-dioxopiperidin-3-yl)-3-oxoisoindolin-4-yl)amino)butyl)piperazin-1-yl)piperidin-1-yl)nicotinoyl)piperidin-4-yl)butyl)-3-(pyridin-3-yl)acrylamide;
(E)-N-(4-(1-(6-(4-(4-(4-((2-(2,6-dioxopiperidin-3-yl)-3-oxoisoindolin-4-yl)oxy)butyl)piperazin-1-yl)piperidin-1-yl)nicotinoyl)piperidin-4-yl)butyl)-3-(pyridin-3-yl)acrylamide;
(E)-N-(4-(1-(6-(4-(4-(4-(2-(2,6-dioxopiperidin-3-yl)-1-oxoisoindolin-5-yl)but-3-yn-1-yl)piperazin-1-yl)piperidin-1-yl)nicotinoyl)piperidin-4-yl)butyl)-3-(pyridin-3-yl)acrylamide;
(E)-N-(4-(1-(6-(4-(4-(7-(2-(2,6-dioxopiperidin-3-yl)-3-oxoisoindolin-5-yl)hept-6-yn-1-yl)piperazin-1-yl)piperidin-1-yl)nicotinoyl)piperidin-4-yl)butyl)-3-(pyridin-3-yl)acrylamide;
(E)-N-(4-(1-(6-(4-(4-(7-((2-(2,6-dioxopiperidin-3-yl)-1-oxoisoindolin-5-yl)thio)heptyl)piperazin-1-yl)piperidin-1-yl)nicotinoyl)piperidin-4-yl)butyl)-3-(pyridin-3-yl)acrylamide;
(E)-N-(4-(1-(6-(4-(4-(6-((2-(2,6-dioxopiperidin-3-yl)-1-oxoisoindolin-5-yl)thio)hexyl)piperazin-1-yl)piperidin-1-yl)nicotinoyl)piperidin-4-yl)butyl)-3-(pyridin-3-yl)acrylamide;
(E)-N-(4-(1-(6-(4-(4-(5-((2-(2,6-dioxopiperidin-3-yl)-1-oxoisoindolin-5-yl)thio)pentyl)piperazin-1-yl)piperidin-1-yl)nicotinoyl)piperidin-4-yl)butyl)-3-(pyridin-3-yl)acrylamide;
(E)-N-(4-(1-(6-(4-(4-(4-((2-(2,6-dioxopiperidin-3-yl)-3-oxoisoindolin-5-yl)ethynyl)benzyl)piperazin-1-yl)piperidin-1-yl)nicotinoyl)piperidin-4-yl)butyl)-3-(pyridin-3-yl)acrylamide;
(E)-N-(4-(1-(6-(4-(4-(4-(2-(2,6-dioxopiperidin-3-yl)-1,3-dioxoisoindolin-5-yl)but-3-yn-1-yl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(7-(2-(2,6-dioxopiperidin-3-yl)-1,3-dioxoisoindolin-5-yl)hept-6-yn-1-yl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(7-((2-(2,6-dioxopiperidin-3-yl)-1,3-dioxoisoindolin-5-yl)thio)heptyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(6-((2-(2,6-dioxopiperidin-3-yl)-1,3-dioxoisoindolin-5-yl)thio)hexyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(5-((2-(2,6-dioxopiperidin-3-yl)-1,3-dioxoisoindolin-5-yl)thio)pentyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(4-((2-(2,6-dioxopiperidin-3-yl)-1,3-dioxoisoindolin-5-yl)ethynyl)benzyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(5-(2-(2,6-dioxopiperidin-3-yl)-3-oxoisoindolin-4-yl)pent-4-yn-1-yl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(4-((2-(2,6-dioxopiperidin-3-yl)-3-oxoisoindolin-4-yl)thio)butyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(4-((2-(2,6-dioxopiperidin-3-yl)-3-oxoisoindolin-4-yl)amino)butyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(4-((2-(2,6-dioxopiperidin-3-yl)-3-oxoisoindolin-4-yl)oxy)butyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(4-(2-(2,6-dioxopiperidin-3-yl)-1-oxoisoindolin-5-yl)but-3-yn-1-yl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(7-(2-(2,6-dioxopiperidin-3-yl)-3-oxoisoindolin-5-yl)hept-6-yn-1-yl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(7-((2-(2,6-dioxopiperidin-3-yl)-1-oxoisoindolin-5-yl)thio)heptyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(6-((2-(2,6-dioxopiperidin-3-yl)-1-oxoisoindolin-5-yl)thio)hexyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(5-((2-(2,6-dioxopiperidin-3-yl)-1-oxoisoindolin-5-yl)thio)pentyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-(4-((2-(2,6-dioxopiperidin-3-yl)-3-oxoisoindolin-5-yl)ethynyl)benzyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(8-(2-(2,6-dioxopiperidin-3-yl)-1,3-dioxoisoindolin-5-yl)oct-7-yn-1-yl)piperazin-1-yl)pyridazine-3-carbonyl)piperidin-4-yl)butyl)-3-(pyridin-3-yl)acrylamide;
(E)-N-(4-(1-(6-(4-(6-(2-(2,6-dioxopiperidin-3-yl)-1,3-dioxoisoindolin-5-yl)hex-5-yn-1-yl)piperazin-1-yl)pyridazine-3-carbonyl)piperidin-4-yl)butyl)-3-(pyridin-3-yl)acrylamide;
(E)-N-(4-(1-(6-(4-(7-((2-(2,6-dioxopiperidin-3-yl)-1,3-dioxoisoindolin-5-yl)thio)heptyl)piperazin-1-yl)pyridazine-3-carbonyl)piperidin-4-yl)butyl)-3-(pyridin-3-yl)acrylamide;
(E)-N-(4-(1-(6-(4-(6-((2-(2,6-dioxopiperidin-3-yl)-1,3-dioxoisoindolin-5-yl)thio)hexyl)piperazin-1-yl)pyridazine-3-carbonyl)piperidin-4-yl)butyl)-3-(pyridin-3-yl)acrylamide;
(E)-N-(4-(1-(6-(4-(5-((2-(2,6-dioxopiperidin-3-yl)-1,3-dioxoisoindolin-5-yl)thio)pentyl)piperazin-1-yl)pyridazine-3-carbonyl)piperidin-4-yl)butyl)-3-(pyridin-3-yl)acrylamide;
(E)-N-(4-(1-(6-(4-(4-((2-(2,6-dioxopiperidin-3-yl)-1,3-dioxoisoindolin-5-yl)ethynyl)benzyl)piperazin-1-yl)pyridazine-3-carbonyl)piperidin-4-yl)butyl)-3-(pyridin-3-yl)acrylamide;
(E)-N-(4-(1-(6-(4-(5-(2-(2,6-dioxopiperidin-3-yl)-3-oxoisoindolin-4-yl)pent-4-yn-1-yl)piperazin-1-yl)pyridazine-3-carbonyl)piperidin-4-yl)butyl)-3-(pyridin-3-yl)acrylamide;
(E)-N-(4-(1-(6-(4-(4-((2-(2,6-dioxopiperidin-3-yl)-3-oxoisoindolin-4-yl)thio)butyl)piperazin-1-yl)pyridazine-3-carbonyl)piperidin-4-yl)butyl)-3-(pyridin-3-yl)acrylamide;
(E)-N-(4-(1-(6-(4-(4-((2-(2,6-dioxopiperidin-3-yl)-3-oxoisoindolin-4-yl)amino)butyl)piperazin-1-yl)pyridazine-3-carbonyl)piperidin-4-yl)butyl)-3-(pyridin-3-yl)acrylamide;
(E)-N-(4-(1-(6-(4-(4-((2-(2,6-dioxopiperidin-3-yl)-3-oxoisoindolin-4-yl)oxy)butyl)piperazin-1-yl)pyridazine-3-carbonyl)piperidin-4-yl)butyl)-3-(pyridin-3-yl)acrylamide;
(E)-N-(4-(1-(6-(4-(8-(2-(2,6-dioxopiperidin-3-yl)-1-oxoisoindolin-5-yl)oct-7-yn-1-yl)piperazin-1-yl)pyridazine-3-carbonyl)piperidin-4-yl)butyl)-3-(pyridin-3-yl)acrylamide;
(E)-N-(4-(1-(6-(4-(6-(2-(2,6-dioxopiperidin-3-yl)-1-oxoisoindolin-5-yl)hex-5-yn-1-yl)piperazin-1-yl)pyridazine-3-carbonyl)piperidin-4-yl)butyl)-3-(pyridin-3-yl)acrylamide;
(E)-N-(4-(1-(6-(4-(7-((2-(2,6-dioxopiperidin-3-yl)-1-oxoisoindolin-5-yl)thio)heptyl)piperazin-1-yl)pyridazine-3-carbonyl)piperidin-4-yl)butyl)-3-(pyridin-3-yl)acrylamide;
(E)-N-(4-(1-(6-(4-(6-((2-(2,6-dioxopiperidin-3-yl)-3-oxoisoindolin-5-yl)thio)hexyl)piperazin-1-yl)pyridazine-3-carbonyl)piperidin-4-yl)butyl)-3-(pyridin-3-yl)acrylamide;
(E)-N-(4-(1-(6-(4-(5-((2-(2,6-dioxopiperidin-3-yl)-1-oxoisoindolin-5-yl)thio)pentyl)piperazin-1-yl)pyridazine-3-carbonyl)piperidin-4-yl)butyl)-3-(pyridin-3-yl)acrylamide;
(E)-N-(4-(1-(6-(4-(4-((2-(2,6-dioxopiperidin-3-yl)-3-oxoisoindolin-5-yl)ethynyl)benzyl)piperazin-1-yl)pyridazine-3-carbonyl)piperidin-4-yl)butyl)-3-(pyridin-3-yl)acrylamide;
(E)-N-(4-(1-(6-(4-(8-(2-(2,6-dioxopiperidin-3-yl)-1,3-dioxoisoindolin-5-yl)oct-7-yn-1-yl)piperazin-1-yl)nicotinoyl)piperidin-4-yl)butyl)-3-(pyridin-3-yl)acrylamide;
(E)-N-(4-(1-(6-(4-(6-(2-(2,6-dioxopiperidin-3-yl)-1,3-dioxoisoindolin-5-yl)hex-5-yn-1-yl)piperazin-1-yl)nicotinoyl)piperidin-4-yl)butyl)-3-(pyridin-3-yl)acrylamide;
(E)-N-(4-(1-(6-(4-(7-((2-(2,6-dioxopiperidin-3-yl)-1,3-dioxoisoindolin-5-yl)thio)heptyl)piperazin-1-yl)nicotinoyl)piperidin-4-yl)butyl)-3-(pyridin-3-yl)acrylamide;
(E)-N-(4-(1-(6-(4-(6-((2-(2,6-dioxopiperidin-3-yl)-1,3-dioxoisoindolin-5-yl)thio)hexyl)piperazin-1-yl)nicotinoyl)piperidin-4-yl)butyl)-3-(pyridin-3-yl)acrylamide;
(E)-N-(4-(1-(6-(4-(5-((2-(2,6-dioxopiperidin-3-yl)-1,3-dioxoisoindolin-5-yl)thio)pentyl)piperazin-1-yl)nicotinoyl)piperidin-4-yl)butyl)-3-(pyridin-3-yl)acrylamide;
(E)-N-(4-(1-(6-(4-(4-((2-(2,6-dioxopiperidin-3-yl)-1,3-dioxoisoindolin-5-yl)ethynyl)benzyl)piperazin-1-yl)nicotinoyl)piperidin-4-yl)butyl)-3-(pyridin-3-yl)acrylamide;
(E)-N-(4-(1-(6-(4-(5-(2-(2,6-dioxopiperidin-3-yl)-3-oxoisoindolin-4-yl)pent-4-yn-1-yl)piperazin-1-yl)nicotinoyl)piperidin-4-yl)butyl)-3-(pyridin-3-yl)acrylamide;
(E)-N-(4-(1-(6-(4-(4-((2-(2,6-dioxopiperidin-3-yl)-3-oxoisoindolin-4-yl)thio)butyl)piperazin-1-yl)nicotinoyl)piperidin-4-yl)butyl)-3-(pyridin-3-yl)acrylamide;
(E)-N-(4-(1-(6-(4-(4-((2-(2,6-dioxopiperidin-3-yl)-3-oxoisoindolin-4-yl)amino)butyl)piperazin-1-yl)nicotinoyl)piperidin-4-yl)butyl)-3-(pyridin-3-yl)acrylamide;
(E)-N-(4-(1-(6-(4-(4-((2-(2,6-dioxopiperidin-3-yl)-3-oxoisoindolin-4-yl)oxy)butyl)piperazin-1-yl)nicotinoyl)piperidin-4-yl)butyl)-3-(pyridin-3-yl)acrylamide;
(E)-N-(4-(1-(6-(4-(8-(2-(2,6-dioxopiperidin-3-yl)-1-oxoisoindolin-5-yl)oct-7-yn-1-yl)piperazin-1-yl)nicotinoyl)piperidin-4-yl)butyl)-3-(pyridin-3-yl)acrylamide;
(E)-N-(4-(1-(6-(4-(6-(2-(2,6-dioxopiperidin-3-yl)-1-oxoisoindolin-5-yl)hex-5-yn-1-yl)piperazin-1-yl)nicotinoyl)piperidin-4-yl)butyl)-3-(pyridin-3-yl)acrylamide;
(E)-N-(4-(1-(6-(4-(7-((2-(2,6-dioxopiperidin-3-yl)-1-oxoisoindolin-5-yl)thio)heptyl)piperazin-1-yl)nicotinoyl)piperidin-4-yl)butyl)-3-(pyridin-3-yl)acrylamide;
(E)-N-(4-(1-(6-(4-(6-((2-(2,6-dioxopiperidin-3-yl)-3-oxoisoindolin-5-yl)thio)hexyl)piperazin-1-yl)nicotinoyl)piperidin-4-yl)butyl)-3-(pyridin-3-yl)acrylamide;
(E)-N-(4-(1-(6-(4-(5-((2-(2,6-dioxopiperidin-3-yl)-1-oxoisoindolin-5-yl)thio)pentyl)piperazin-1-yl)nicotinoyl)piperidin-4-yl)butyl)-3-(pyridin-3-yl)acrylamide;
(E)-N-(4-(1-(6-(4-(4-((2-(2,6-dioxopiperidin-3-yl)-3-oxoisoindolin-5-yl)ethynyl)benzyl)piperazin-1-yl)nicotinoyl)piperidin-4-yl)butyl)-3-(pyridin-3-yl)acrylamide;
(E)-N-(4-(1-(4-(4-(8-(2-(2,6-dioxopiperidin-3-yl)-1,3-dioxoisoindolin-5-yl)oct-7-yn-1-yl)piperazin-1-yl)benzoyl)piperidin-4-yl)butyl)-3-(pyridin-3-yl)acrylamide;
(E)-N-(4-(1-(4-(4-(6-(2-(2,6-dioxopiperidin-3-yl)-1,3-dioxoisoindolin-5-yl)hex-5-yn-1-yl)piperazin-1-yl)benzoyl)piperidin-4-yl)butyl)-3-(pyridin-3-yl)acrylamide;
(E)-N-(4-(1-(4-(4-(7-((2-(2,6-dioxopiperidin-3-yl)-1,3-dioxoisoindolin-5-yl)thio)heptyl)piperazin-1-yl)benzoyl)piperidin-4-yl)butyl)-3-(pyridin-3-yl)acrylamide;
(E)-N-(4-(1-(4-(4-(6-((2-(2,6-dioxopiperidin-3-yl)-1,3-dioxoisoindolin-5-yl)thio)hexyl)piperazin-1-yl)benzoyl)piperidin-4-yl)butyl)-3-(pyridin-3-yl)acrylamide;
(E)-N-(4-(1-(4-(4-(5-((2-(2,6-dioxopiperidin-3-yl)-1,3-dioxoisoindolin-5-yl)thio)pentyl)piperazin-1-yl)benzoyl)piperidin-4-yl)butyl)-3-(pyridin-3-yl)acrylamide;
(E)-N-(4-(1-(4-(4-(4-((2-(2,6-dioxopiperidin-3-yl)-1,3-dioxoisoindolin-5-yl)ethynyl)benzyl)piperazin-1-yl)benzoyl)piperidin-4-yl)butyl)-3-(pyridin-3-yl)acrylamide;
(E)-N-(4-(1-(4-(4-(5-(2-(2,6-dioxopiperidin-3-yl)-3-oxoisoindolin-4-yl)pent-4-yn-1-yl)piperazin-1-yl)benzoyl)piperidin-4-yl)butyl)-3-(pyridin-3-yl)acrylamide;
(E)-N-(4-(1-(4-(4-(4-((2-(2,6-dioxopiperidin-3-yl)-3-oxoisoindolin-4-yl)thio)butyl)piperazin-1-yl)benzoyl)piperidin-4-yl)butyl)-3-(pyridin-3-yl)acrylamide;
(E)-N-(4-(1-(4-(4-(4-((2-(2,6-dioxopiperidin-3-yl)-3-oxoisoindolin-4-yl)amino)butyl)piperazin-1-yl)benzoyl)piperidin-4-yl)butyl)-3-(pyridin-3-yl)acrylamide;
(E)-N-(4-(1-(4-(4-(4-((2-(2,6-dioxopiperidin-3-yl)-3-oxoisoindolin-4-yl)oxy)butyl)piperazin-1-yl)benzoyl)piperidin-4-yl)butyl)-3-(pyridin-3-yl)acrylamide;
(E)-N-(4-(1-(4-(4-(8-(2-(2,6-dioxopiperidin-3-yl)-1-oxoisoindolin-5-yl)oct-7-yn-1-yl)piperazin-1-yl)benzoyl)piperidin-4-yl)butyl)-3-(pyridin-3-yl)acrylamide;
(E)-N-(4-(1-(4-(4-(6-(2-(2,6-dioxopiperidin-3-yl)-1-oxoisoindolin-5-yl)hex-5-yn-1-yl)piperazin-1-yl)benzoyl)piperidin-4-yl)butyl)-3-(pyridin-3-yl)acrylamide;
(E)-N-(4-(1-(4-(4-(7-((2-(2,6-dioxopiperidin-3-yl)-1-oxoisoindolin-5-yl)thio)heptyl)piperazin-1-yl)benzoyl)piperidin-4-yl)butyl)-3-(pyridin-3-yl)acrylamide;
(E)-N-(4-(1-(4-(4-(6-((2-(2,6-dioxopiperidin-3-yl)-3-oxoisoindolin-5-yl)thio)hexyl)piperazin-1-yl)benzoyl)piperidin-4-yl)butyl)-3-(pyridin-3-yl)acrylamide;
(E)-N-(4-(1-(4-(4-(5-((2-(2,6-dioxopiperidin-3-yl)-1-oxoisoindolin-5-yl)thio)pentyl)piperazin-1-yl)benzoyl)piperidin-4-yl)butyl)-3-(pyridin-3-yl)acrylamide;
(E)-N-(4-(1-(4-(4-(4-((2-(2,6-dioxopiperidin-3-yl)-3-oxoisoindolin-5-yl)ethynyl)benzyl)piperazin-1-yl)benzoyl)piperidin-4-yl)butyl)-3-(pyridin-3-yl)acrylamide;
(E)-N-(4-(1-(4-(1-(8-(2-(2,6-dioxopiperidin-3-yl)-1,3-dioxoisoindolin-5-yl)oct-7-yn-1-yl)piperidin-4-yl)benzoyl)piperidin-4-yl)butyl)-3-(pyridin-3-yl)acrylamide;
(E)-N-(4-(1-(4-(1-(6-(2-(2,6-dioxopiperidin-3-yl)-1,3-dioxoisoindolin-5-yl)hex-5-yn-1-yl)piperidin-4-yl)benzoyl)piperidin-4-yl)butyl)-3-(pyridin-3-yl)acrylamide;
(E)-N-(4-(1-(4-(1-(7-((2-(2,6-dioxopiperidin-3-yl)-1,3-dioxoisoindolin-5-yl)thio)heptyl)piperidin-4-yl)benzoyl)piperidin-4-yl)butyl)-3-(pyridin-3-yl)acrylamide;
(E)-N-(4-(1-(4-(1-(6-((2-(2,6-dioxopiperidin-3-yl)-1,3-dioxoisoindolin-5-yl)thio)hexyl)piperidin-4-yl)benzoyl)piperidin-4-yl)butyl)-3-(pyridin-3-yl)acrylamide;
(E)-N-(4-(1-(4-(1-(5-((2-(2,6-dioxopiperidin-3-yl)-1,3-dioxoisoindolin-5-yl)thio)pentyl)piperidin-4-yl)benzoyl)piperidin-4-yl)butyl)-3-(pyridin-3-yl)acrylamide;
(E)-N-(4-(1-(4-(1-(4-((2-(2,6-dioxopiperidin-3-yl)-1,3-dioxoisoindolin-5-yl)ethynyl)benzyl)piperidin-4-yl)benzoyl)piperidin-4-yl)butyl)-3-(pyridin-3-yl)acrylamide;
(E)-N-(4-(1-(4-(1-(5-(2-(2,6-dioxopiperidin-3-yl)-3-oxoisoindolin-4-yl)pent-4-yn-1-yl)piperidin-4-yl)benzoyl)piperidin-4-yl)butyl)-3-(pyridin-3-yl)acrylamide;
(E)-N-(4-(1-(4-(1-(4-((2-(2,6-dioxopiperidin-3-yl)-3-oxoisoindolin-4-yl)thio)butyl)piperidin-4-yl)benzoyl)piperidin-4-yl)butyl)-3-(pyridin-3-yl)acrylamide;
(E)-N-(4-(1-(4-(1-(4-((2-(2,6-dioxopiperidin-3-yl)-3-oxoisoindolin-4-yl)amino)butyl)piperidin-4-yl)benzoyl)piperidin-4-yl)butyl)-3-(pyridin-3-yl)acrylamide;
(E)-N-(4-(1-(4-(1-(4-((2-(2,6-dioxopiperidin-3-yl)-3-oxoisoindolin-4-yl)oxy)butyl)piperidin-4-yl)benzoyl)piperidin-4-yl)butyl)-3-(pyridin-3-yl)acrylamide;
(E)-N-(4-(1-(4-(1-(8-(2-(2,6-dioxopiperidin-3-yl)-1-oxoisoindolin-5-yl)oct-7-yn-1-yl)piperidin-4-yl)benzoyl)piperidin-4-yl)butyl)-3-(pyridin-3-yl)acrylamide;
(E)-N-(4-(1-(4-(1-(6-(2-(2,6-dioxopiperidin-3-yl)-1-oxoisoindolin-5-yl)hex-5-yn-1-yl)piperidin-4-yl)benzoyl)piperidin-4-yl)butyl)-3-(pyridin-3-yl)acrylamide;
(E)-N-(4-(1-(4-(1-(7-((2-(2,6-dioxopiperidin-3-yl)-1-oxoisoindolin-5-yl)thio)heptyl)piperidin-4-yl)benzoyl)piperidin-4-yl)butyl)-3-(pyridin-3-yl)acrylamide;
(E)-N-(4-(1-(4-(1-(6-((2-(2,6-dioxopiperidin-3-yl)-3-oxoisoindolin-5-yl)thio)hexyl)piperidin-4-yl)benzoyl)piperidin-4-yl)butyl)-3-(pyridin-3-yl)acrylamide;
(E)-N-(4-(1-(4-(1-(5-((2-(2,6-dioxopiperidin-3-yl)-1-oxoisoindolin-5-yl)thio)pentyl)piperidin-4-yl)benzoyl)piperidin-4-yl)butyl)-3-(pyridin-3-yl)acrylamide;
(E)-N-(4-(1-(4-(1-(4-((2-(2,6-dioxopiperidin-3-yl)-3-oxoisoindolin-5-yl)ethynyl)benzyl)piperidin-4-yl)benzoyl)piperidin-4-yl)butyl)-3-(pyridin-3-yl)acrylamide;
(E)-N-(4-(1-(4-(4-(7-(2-(2,6-dioxopiperidin-3-yl)-1-oxoisoindolin-4-yl)hept-6-yn-1-yl)piperazin-1-yl)benzoyl)piperidin-4-yl)butyl)-3-(pyridazin-4-yl)acrylamide;
(E)-N-(4-(1-(4-(4-(7-(2-(2,6-dioxopiperidin-3-yl)-1-oxoisoindolin-4-yl)hept-6-yn-1-yl)piperazin-1-yl)benzoyl)piperidin-4-yl)butyl)-3-(1,2,3-triazin-5-yl)acrylamide;
(E)-N-(4-(1-(4-(4-(7-(2-(2,6-dioxopiperidin-3-yl)-1-oxoisoindolin-4-yl)hept-6-yn-1-yl)piperazin-1-yl)benzoyl)piperidin-4-yl)butyl)-3-(pyrazin-2-yl)acrylamide;
(E)-N-(4-(1-(4-(4-(7-(2-(2,6-dioxopiperidin-3-yl)-1-oxoisoindolin-4-yl)hept-6-yn-1-yl)piperazin-1-yl)benzoyl)piperidin-4-yl)butyl)-3-(1H-pyrrol-3-yl)acrylamide;
(E)-3-([1,2,4]triazolo[4,3-a]pyridin-6-yl)-N-(4-(1-(4-(4-(7-(2-(2,6-dioxopiperidin-3-yl)-1-oxoisoindolin-4-yl)hept-6-yn-1-yl)piperazin-1-yl)benzoyl)piperidin-4-yl)butyl)acrylamide;
(E)-N-(4-(1-(4-(4-(7-(2-(2,6-dioxopiperidin-3-yl)-1-oxoisoindolin-4-yl)hept-6-yn-1-yl)piperazin-1-yl)benzoyl)piperidin-4-yl)butyl)-3-(1H-pyrazolo[3,4-b]pyridin-5-yl)acrylamide;
(E)-N-(4-(4-(4-(4-(7-(2-(2,6-dioxopiperidin-3-yl)-1-oxoisoindolin-4-yl)hept-6-yn-1-yl)piperazin-1-yl)benzoyl)-1,4-diazepan-1-yl)butyl)-3-(6-fluoropyridin-3-yl)acrylamide;
(E)-N-(4-(4-(4-(4-(7-(2-(2,6-dioxopiperidin-3-yl)-1-oxoisoindolin-4-yl)hept-6-yn-1-yl)piperazin-1-yl)benzoyl)-1,4-diazepan-1-yl)butyl)-3-(2-fluoropyridin-3-yl)acrylamide;
(E)-2-cyano-N-(4-(1-(4-(4-(7-(2-(2,6-dioxopiperidin-3-yl)-1-oxoisoindolin-4-yl)hept-6-yn-1-yl)piperazin-1-yl)benzoyl)pyrrolidin-3-yl)butyl)-3-(2-fluoropyridin-3-yl)acrylamide;
(E)-N-(4-(1-(4-(4-(7-(2-(2,6-dioxopiperidin-3-yl)-1-oxoisoindolin-4-yl)hept-6-yn-1-yl)piperazin-1-yl)benzoyl)pyrrolidin-3-yl)butyl)-3-(2-fluoropyridin-3-yl)acrylamide;
(E)-N-(4-(1-(4-(4-(7-(2-(2,6-dioxopiperidin-3-yl)-1-oxoisoindolin-4-yl)hept-6-yn-1-yl)piperazin-1-yl)-2-fluorobenzoyl)pyrrolidin-3-yl)butyl)-3-(5-fluoropyridin-3-yl)acrylamide;
(E)-N-(4-(1-(4-(4-(7-(2-(2,6-dioxopiperidin-3-yl)-1-oxoisoindolin-4-yl)hept-6-yn-1-yl)piperazin-1-yl)-2-fluorobenzoyl)pyrrolidin-3-yl)butyl)-3-(pyridin-3-yl)acrylamide;
(E)-N-(4-(1-(4-(4-(7-(2-(2,6-dioxopiperidin-3-yl)-1-oxoisoindolin-4-yl)hept-6-yn-1-yl)piperazin-1-yl)-2-fluorobenzoyl)pyrrolidin-3-yl)butyl)-3-(5-methylpyridin-3-yl)acrylamide;
(E)-N-(4-(1-(4-(4-(4-((2-(2,6-dioxopiperidin-3-yl)-1-oxoisoindolin-4-yl)thio)butyl)piperazin-1-yl)-3-methylbenzoyl)piperidin-4-yl)butyl)-3-(5-methylpyridin-3-yl)acrylamide;
(E)-N-(4-(1-(3-cyclopropyl-4-(4-(4-((2-(2,6-dioxopiperidin-3-yl)-1-oxoisoindolin-4-yl)thio)butyl)piperazin-1-yl)benzoyl)piperidin-4-yl)butyl)-3-(6-methylpyrazin-2-yl)acrylamide;
(Z)-2-chloro-N-(4-(1-(5-(4-(4-((2-(2,6-dioxopiperidin-3-yl)-1-oxoisoindolin-4-yl)thio)butyl)piperazin-1-yl)-4-ethylpicolinoyl)piperidin-4-yl)butyl)-3-(6-methylpyrazin-2-yl)acrylamide;
(E)-N-(4-(1-(5-(3-(4-((2-(2,6-dioxopiperidin-3-yl)-1-oxoisoindolin-4-yl)thio)butyl)imidazolidin-1-yl)picolinoyl)piperidin-4-yl)butyl)-3-(6-methylpyrazin-2-yl)acrylamide;
(E)-N-(4-(1-(5-(3-(4-((2-(2,6-dioxopiperidin-3-yl)-1-oxoisoindolin-4-yl)thio)butyl)-5-methylimidazolidin-1-yl)picolinoyl)piperidin-4-yl)butyl)-3-(6-methylpyrazin-2-yl)acrylamide;
(E)-2-cyano-N-(4-(1-(4-(4-(7-(2-(2,6-dioxopiperidin-3-yl)-1-oxoisoindolin-4-yl)hept-6-yn-1-yl)piperazin-1-yl)benzoyl)piperidin-4-yl)butyl)-3-(pyridazin-4-yl)acrylamide;
(E)-2-cyano-N-(4-(1-(4-(4-(7-(2-(2,6-dioxopiperidin-3-yl)-1-oxoisoindolin-4-yl)hept-6-yn-1-yl)piperazin-1-yl)benzoyl)piperidin-4-yl)butyl)-3-(pyridin-3-yl)acrylamide;
(Z)-3-cyano-N-(3-(1-(4-(4-(7-(2-(2,6-dioxopiperidin-3-yl)-1-oxoisoindolin-4-yl)hept-6-yn-1-yl)piperazin-1-yl)benzoyl)piperidin-4-yl)propyl)-3-(pyridin-3-yl)acrylamide;
(E)-2-cyano-N-(4-(1-(4-(4-(7-(2-(2,6-dioxopiperidin-3-yl)-1-oxoisoindolin-4-yl)hept-6-yn-1-yl)piperazin-1-yl)benzoyl)piperidin-4-yl)butyl)-3-(6-fluoropyridin-3-yl)acrylamide;
(E)-N-(4-(1-(4-(4-(7-(2-(2,6-dioxopiperidin-3-yl)-1-oxoisoindolin-4-yl)hept-6-yn-1-yl)piperazin-1-yl)benzoyl)piperidin-4-yl)butyl)-3-(6-fluoropyridin-3-yl)acrylamide;
(E)-N-(4-(1-(4-(4-(7-(2-(2,6-dioxopiperidin-3-yl)-1-oxoisoindolin-4-yl)hept-6-yn-1-yl)piperazin-1-yl)benzoyl)piperidin-4-yl)butyl)-3-(2-fluoropyridin-3-yl)acrylamide;
(E)-2-cyano-N-(4-(4-(4-(4-(7-(2-(2,6-dioxopiperidin-3-yl)-1-oxoisoindolin-4-yl)hept-6-yn-1-yl)piperazin-1-yl)benzoyl)-6-methyl-1,4-diazepan-1-yl)butyl)-3-(2-fluoropyridin-3-yl)acrylamide;
(E)-N-(4-(4-(4-(4-(7-(2-(2,6-dioxopiperidin-3-yl)-1-oxoisoindolin-4-yl)hept-6-yn-1-yl)piperazin-1-yl)benzoyl)-6-methyl-1,4-diazepan-1-yl)butyl)-3-(2-fluoropyridin-3-yl)acrylamide;
(E)-N-(4-(1-(4-(4-(7-(2-(2,6-dioxopiperidin-3-yl)-1-oxoisoindolin-4-yl)hept-6-yn-1-yl)piperazin-1-yl)benzoyl)pyrrolidin-3-yl)butyl)-3-(6-fluoropyridin-3-yl)acrylamide;
(E)-N-(4-(1-(4-(4-(7-(2-(2,6-dioxopiperidin-3-yl)-1-oxoisoindolin-4-yl)hept-6-yn-1-yl)piperazin-1-yl)benzoyl)pyrrolidin-3-yl)butyl)-3-(pyridin-3-yl)acrylamide;
(E)-N-(4-(1-(4-(4-(7-(2-(2,6-dioxopiperidin-3-yl)-1-oxoisoindolin-4-yl)hept-6-yn-1-yl)piperazin-1-yl)-2-fluorobenzoyl)pyrrolidin-3-yl)butyl)-3-(5-fluoropyridin-3-yl)acrylamide;
(E)-N-(4-(1-(4-(4-(4-((2-(2,6-dioxopiperidin-3-yl)-1-oxoisoindolin-4-yl)thio)butyl)piperazin-1-yl)-3-hydroxybenzoyl)piperidin-4-yl)butyl)-3-(5-fluoropyridin-3-yl)acrylamide;
(E)-N-(4-(1-(4-(4-(4-((2-(2,6-dioxopiperidin-3-yl)-1-oxoisoindolin-4-yl)thio)butyl)piperazin-1-yl)-3-methylbenzoyl)piperidin-4-yl)butyl)-3-(5-fluoropyridin-3-yl)acrylamide;
(E)-N-(4-(1-(4-(4-(4-((2-(2,6-dioxopiperidin-3-yl)-1-oxoisoindolin-4-yl)thio)butyl)piperazin-1-yl)-3-methylbenzoyl)piperidin-4-yl)butyl)-3-(6-methylpyrazin-2-yl)acrylamide;
(Z)—N-(4-(1-(3-cyclopropyl-4-(4-(4-((2-(2,6-dioxopiperidin-3-yl)-1-oxoisoindolin-4-yl)thio)butyl)piperazin-1-yl)benzoyl)piperidin-4-yl)butyl)-3-fluoro-3-(6-methylpyrazin-2-yl)acrylamide;
(2S,4R)-1-((S)-2-(6-(4-(5-(4-(4-((Z)-2-chloro-3-(6-methylpyrazin-2-yl)acrylamido)butyl)piperidine-1-carbonyl)pyridin-2-yl)piperazin-1-yl)-6-oxohexanamido)-3,3-dimethylbutanoyl)-4-hydroxy-N—((S)-1-(4-(4-methylthiazol-5-yl)phenyl)ethyl)pyrrolidine-2-carboxamide;
(E)-N-(4-(1-(5-(3-(4-((2-(2,6-dioxopiperidin-3-yl)-1-oxoisoindolin-4-yl)amino)butyl)imidazolidin-1-yl)picolinoyl)piperidin-4-yl)butyl)-3-(6-methylpyrazin-2-yl)acrylamide;
(E)-N-(4-(1-(4-(4-(4-((2-(2,6-dioxopiperidin-3-yl)-1-oxoisoindolin-4-yl)thio)butyl)-2-methylpiperazin-1-yl)-3-isopropylbenzoyl)piperidin-4-yl)butyl)-3-(pyrazin-2-yl)acrylamide;
(E)-N-(4-(1-(4-(4-((2-(2,6-dioxopiperidin-3-yl)-1-oxoisoindolin-5-yl)methyl)piperazin-1-yl)benzoyl)piperidin-4-yl)butyl)-3-(pyridin-3-yl)acrylamide;
(E)-N-(4-(1-(4-(4-((4-(2-(2,6-dioxopiperidin-3-yl)-4-fluoro-1,3-dioxoisoindolin-5-yl)piperazin-1-yl)methyl)piperazin-1-yl)benzoyl)piperidin-4-yl)butyl)-3-(pyridin-3-yl)acrylamide;
(E)-N-(4-(1-(4-(4-((4-(2-(2,6-dioxopiperidin-3-yl)-1,3-dioxoisoindolin-5-yl)piperazin-1-yl)methyl)piperazin-1-yl)benzoyl)piperidin-4-yl)butyl)-3-(pyridin-3-yl)acrylamide;
(E)-N-(4-(1-(4-(1-((4-(2-(2,6-dioxopiperidin-3-yl)-1,3-dioxoisoindolin-5-yl)piperazin-1-yl)methyl)piperidin-4-yl)benzoyl)piperidin-4-yl)butyl)-3-(pyridin-3-yl)acrylamide;
(E)-N-(4-(1-(6-(4-(5-(4-(2-(2,6-dioxopiperidin-3-yl)-1-oxoisoindolin-5-yl)piperazin-1-yl)pentyl)piperazin-1-yl)pyridazine-3-carbonyl)piperidin-4-yl)butyl)-3-(pyridin-3-yl)acrylamide;
(E)-N-(4-(1-(6-(4-(4-((4-(2-(2,6-dioxopiperidin-3-yl)-1-oxoisoindolin-5-yl)piperazin-1-yl)methyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-((2-(2,6-dioxopiperidin-3-yl)-1-oxoisoindolin-5-yl)methyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-((2-(2,6-dioxopiperidin-3-yl)-1,3-dioxoisoindolin-5-yl)methyl)piperazin-1-yl)piperidin-1-yl)pyridazine-3-carbonyl)piperidin-4-yl)butyl)-3-(pyridin-3-yl)acrylamide;
(E)-N-(4-(1-(6-(4-(4-((2-(2,6-dioxopiperidin-3-yl)-1-oxoisoindolin-5-yl)methyl)piperazin-1-yl)piperidin-1-yl)pyridazine-3-carbonyl)piperidin-4-yl)butyl)-3-(pyridin-3-yl)acrylamide;
(E)-N-((E)-4-(1-(4-(4-(7-(2-(2,6-dioxopiperidin-3-yl)-1-oxoisoindolin-4-yl)hept-6-yn-1-yl)piperazin-1-yl)benzoyl)piperidin-4-yl)but-2-en-1-yl)-3-(pyridazin-4-yl)acrylamide;
(E)-N-((E)-4-(1-(4-(4-(7-(2-(2,6-dioxopiperidin-3-yl)-1-oxoisoindolin-4-yl)hept-6-yn-1-yl)piperazin-1-yl)benzoyl)piperidin-4-yl)but-2-en-1-yl)-3-(pyridin-3-yl)acrylamide;
(E)-N-((E)-4-(1-(4-(4-(4-((2-(2,6-dioxopiperidin-3-yl)-1-oxoisoindolin-4-yl)thio)butyl)piperazin-1-yl)benzoyl)piperidin-4-yl)but-2-en-1-yl)-3-(pyridin-3-yl)acrylamide;
(E)-N-((E)-4-(1-(4-(4-(5-(2-(2,6-dioxopiperidin-3-yl)-1-oxoisoindolin-4-yl)pent-4-yn-1-yl)piperazin-1-yl)benzoyl)piperidin-4-yl)but-2-en-1-yl)-3-(pyridin-3-yl)acrylamide;
(E)-N-((E)-4-(1-(4-(4-(6-(2-(2,6-dioxopiperidin-3-yl)-1-oxoisoindolin-4-yl)hex-5-yn-1-yl)piperazin-1-yl)benzoyl)piperidin-4-yl)but-2-en-1-yl)-3-(pyridin-3-yl)acrylamide;
(E)-N-((E)-4-(1-(4-(4-(8-(2-(2,6-dioxopiperidin-3-yl)-1-oxoisoindolin-4-yl)oct-7-yn-1-yl)piperazin-1-yl)benzoyl)piperidin-4-yl)but-2-en-1-yl)-3-(pyridin-3-yl)acrylamide;
(E)-N-(4-(1-(4-(4-(4-(1-(2-(2,6-dioxopiperidin-3-yl)-1-oxoisoindolin-4-yl)-1H-1,2,3-triazol-4-yl)butyl)piperazin-1-yl)benzoyl)piperidin-4-yl)butyl)-3-(pyridin-3-yl)acrylamide;
(E)-N-(4-(1-(4-(4-(3-(1-(2-(2,6-dioxopiperidin-3-yl)-1-oxoisoindolin-4-yl)-1H-1,2,3-triazol-4-yl)propyl)piperazin-1-yl)benzoyl)piperidin-4-yl)butyl)-3-(pyridin-3-yl)acrylamide;
(E)-N-(4-(1-(4-((2-((2-(2,6-dioxopiperidin-3-yl)-1-oxoisoindolin-4-yl)thio)ethyl)amino)benzoyl)piperidin-4-yl)butyl)-3-(pyridin-3-yl)acrylamide;
(E)-N-(4-(1-(4-((4-((2-(2,6-dioxopiperidin-3-yl)-1-oxoisoindolin-4-yl)thio)butyl)amino)benzoyl)piperidin-4-yl)butyl)-3-(pyridin-3-yl)acrylamide;
(E)-N-(4-(1-(4-((7-(2-(2,6-dioxopiperidin-3-yl)-1-oxoisoindolin-4-yl)hept-6-yn-1-yl)amino)benzoyl)piperidin-4-yl)butyl)-3-(pyridin-3-yl)acrylamide;
(E)-N-(4-(1-(4-(4-(7-(2-(2,6-dioxopiperidin-3-yl)-1-oxoisoindolin-4-yl)hept-6-yn-1-yl)piperazin-1-yl)benzoyl)piperidin-4-yl)butyl)-3-(2-fluoropyridin-3-yl)acrylamide;
(E)-N-(4-(1-(4-(4-(6-((2-(2,6-dioxopiperidin-3-yl)-1-oxoisoindolin-4-yl)oxy)hexyl)piperazin-1-yl)benzoyl)piperidin-4-yl)butyl)-3-(pyridin-3-yl)acrylamide;
(E)-N-(4-(1-(4-(4-(7-((2-(2,6-dioxopiperidin-3-yl)-1-oxoisoindolin-4-yl)oxy)heptyl)piperazin-1-yl)benzoyl)piperidin-4-yl)butyl)-3-(pyridin-3-yl)acrylamide;
(E)-N-(4-(1-(6-(4-(4-(7-(2-(2,6-dioxopiperidin-3-yl)-1-oxoisoindolin-4-yl)hept-6-yn-1-yl)piperazin-1-yl)piperidin-1-yl)nicotinoyl)piperidin-4-yl)butyl)-3-(pyridin-3-yl)acrylamide;
(E)-N-(4-(1-(6-(4-(4-(8-(2-(2,6-dioxopiperidin-3-yl)-1-oxoisoindolin-4-yl)oct-7-yn-1-yl)piperazin-1-yl)piperidin-1-yl)nicotinoyl)piperidin-4-yl)butyl)-3-(pyridin-3-yl)acrylamide; and
(2S,4R)-1-((S)-3,3-dimethyl-2-(6-oxo-6-(4-(1-(6-(4-(4-((E)-3-(pyridin-3-yl)acrylamido)butyl)piperidine-1-carbonyl)pyridazin-3-yl)piperidin-4-yl)piperazin-1-yl)hexanamido)butanoyl)-4-hydroxy-N-(4-(4-methylthiazol-5-yl)benzyl)pyrrolidine-2-carboxamide.

25. The compound of Formula (I) or a salt, enantiomer, stereoisomer, solvate, isotopically enriched analog, prodrug, or polymorph thereof as claimed in claim 1, which is a hydrochloride, sulfate, citrate, maleate, sulfonate, citrate, lactate, tartrate, fumarate, phosphate, dihydrogenphosphate, pyrophosphate, metaphosphate, oxalate, malonate, benzoate, mandelate, succinate, trifluoroacetate, glycolate, or p-toluenesulfonate of the compound of Formula (I).

26. A pharmaceutical composition comprising the compound of Formula (I) or a pharmaceutically acceptable salt thereof as claimed in claim 1, and at least one pharmaceutically acceptable carrier.

27. The pharmaceutical composition of claim 26, further comprising at least one additional therapeutic agent.

28. A medicine kit or reagent kit comprising the compound of Formula (I) or a pharmaceutically acceptable salt thereof as claimed in claim 1, or a pharmaceutical composition comprising the compound of Formula (I) or a pharmaceutically acceptable salt and at least one pharmaceutically acceptable carrier.

29-31. (canceled)

32. The compound of Formula (I) or a pharmaceutically acceptable salt thereof as claimed in claim 1, for use as a medicament.

33. The compound of Formula (I) or a pharmaceutically acceptable salt thereof as claimed in claim 1, for use in the prevention and/or treatment of a disease or disorder associated with NAMPT.

34. The compound of Formula (I) or a pharmaceutically acceptable salt thereof as claimed in claim 33, wherein the disease or disorder associated with NAMPT comprises: tumors, autoimmune diseases, inflammatory diseases, gestational hypertension, cardiovascular and cerebrovascular diseases, obesity and diabetic nephropathy.

35. The compound of Formula (I) or a pharmaceutically acceptable salt thereof as claimed in claim 33, wherein the disease or disorder associated with NAMPT comprises: colorectal cancer; breast cancer (including triple negative breast cancer, invasive breast cancer, infiltrating breast cancer); astrocytoma; pancreatic cancer; gastric cancer; prostate cancer; melanoma; leukemia (such as acute myeloid leukemia, acute lymphocytic leukemia); ovarian cancer; liver cancer; lung cancer (such as non-small cell lung cancer and small cell lung cancer); glioblastoma; multiple myeloma; esophageal cancer; bladder cancer; thyroid cancer; endometrial cancer; lymphoma (such as diffuse large B-cell carcinoma, follicular B-cell lymphoma, Hodgkin's lymphoma, peripheral T-cell lymphoma); neuroendocrine tumors; renal cancer (such as renal oncocytoma, clear cell renal cell carcinoma, renal urothelial carcinoma); pediatric gliomas; rhabdomyosarcoma; leiomyosarcoma; urothelial carcinoma; basal cell carcinoma; oral squamous cell carcinoma; cholangiocarcinoma; bone cancer; cervical cancer; skin cancer; oral squamous cell carcinoma; autoimmune diseases (such as rheumatoid arthritis, autoimmune encephalitis); cardiovascular and cerebrovascular diseases (including coronary atherosclerosis, acute myocardial infarction, myocardial ischemia-reperfusion injury, ischemic stroke); inflammatory diseases; gestational hypertension; obesity; and diabetic nephropathy.

36. A method for treating or preventing a disease or disorder associated with NAMPT in a subject, comprising administering to the subject a therapeutically effective amount of the compound of Formula (I) or a pharmaceutically acceptable salt thereof as claimed in claim 1, or a pharmaceutical composition comprising the compound of Formula (I) or a pharmaceutically acceptable salt and at least one pharmaceutically acceptable carrier.

37. The method as claimed in claim 36, wherein the disease or disorder associated with NAMPT comprises: tumors, autoimmune diseases, inflammatory diseases, gestational hypertension, cardiovascular and cerebrovascular diseases, obesity and diabetic nephropathy.

38. The method as claimed in claim 36, wherein the disease or disorder associated with NAMPT comprises:
colorectal cancer; breast cancer (including triple negative breast cancer, invasive breast cancer, infiltrating breast cancer); astrocytoma; pancreatic cancer; gastric cancer; prostate cancer; melanoma; leukemia (such as acute myeloid leukemia, acute lymphocytic leukemia); ovarian cancer; liver cancer; lung cancer (such as non-small cell lung cancer and small cell lung cancer); glioblastoma; multiple myeloma; esophageal cancer; bladder cancer; thyroid cancer; endometrial cancer; lymphoma (such as diffuse large B-cell carcinoma, follicular B-cell lymphoma, Hodgkin's lymphoma, peripheral T-cell lymphoma); neuroendocrine tumors; renal cancer (such as renal oncocytoma, clear cell renal cell carcinoma, renal urothelial carcinoma); pediatric gliomas; rhabdomyosarcoma; leiomyosarcoma; urothelial carcinoma; basal cell carcinoma; oral squamous cell carcinoma; cholangiocarcinoma; bone cancer; cervical cancer; skin cancer; oral squamous cell carcinoma; autoimmune diseases (such as rheumatoid arthritis, autoimmune encephalitis); cardiovascular and cerebrovascular diseases (including coronary atherosclerosis, acute myocardial infarction, myocardial ischemia-reperfusion injury, ischemic stroke); inflammatory diseases; gestational hypertension; obesity; and diabetic nephropathy.

39. The method as claimed in claim 36, wherein the compound of Formula (I) or a pharmaceutically acceptable salt thereof or the pharmaceutical composition is administered to the subject through at least one mode of administration selected from the group consisting of nasal administration, inhalation administration, topical administration, oral administration, oral mucosal administration, rectal administration, pleural cavity administration, peritoneal administration, vaginal administration, intramuscular administration, subcutaneous, transdermal, epidural, intrathecal, and intravenous administration.
